(19) United States
(12) Patent Application Publication
(10) Pub. No.: US 2023/0416228 A1
(43) Pub. Date: Dec. 28, 2023

(54) AGENTS FOR THE TREATMENT OF DISEASES BY INHIBITION OF FOXO1

(71) Applicant: Forkhead Biotherapeutics, Inc., New York, NY (US)

(72) Inventors: Sandro Belvedere, New York, NY (US); Hua Viola Lin, Zionsville, IN (US); Robert J. DeVita, Westfield, NJ (US); Stephane Turcotte, Saint-Laurent (CA); Shawn Johnstone, Saint-Lazare (CA)

(21) Appl. No.: 18/178,192

(22) Filed: Mar. 3, 2023

Related U.S. Application Data
(63) Continuation of application No. PCT/US21/51790, filed on Sep. 23, 2021.
(60) Provisional application No. 63/082,874, filed on Sep. 24, 2020.

(51) Int. Cl. C07D 401/14; C07D 403/04

(52) U.S. Cl. CPC C07D 401/14; C07D 403/04

(57) ABSTRACT

The disclosure provides FOXO1 inhibitors having beneficial properties such as selectivity and metabolic stability. FOXO1 inhibitors are useful in the treatment of diabetes.

### CROSS-REFERENCE

This application is a continuation of International Application No. PCT/US21/51790, filed Sep. 23, 2021, which claims the benefit of U.S. Provisional Application No. 63/082,874, filed Sep. 24, 2020, which is incorporated herein by reference in its entirety.

### STATEMENT REGARDING FEDERALLY SPONSORED RESEARCH

This invention was made with government support under 1R43DK120177-01A1 awarded by the National Institutes of Health (NIH). The government has certain rights in the invention.

### BACKGROUND

FOX (forkhead box) proteins are a family of transcription factors, defined by the presence of the forkhead box, a sequence of 80 to 100 amino acids forming a DNA-binding motif. Forkhead proteins are a subgroup of the helix-turn-helix class of proteins. The forkhead motif is also known as the winged helix due to the butterfly-like appearance of the loops in the protein structure of the domain.

More than one hundred FOX members exist in human, grouped in sub classes from FOXA to FOXR. The FOXO class of FOX members plays an important role in cellular homeostasis, regulating apoptosis, cell-cycle progression and oxidative stress resistance. The activity of FOXOs is tightly regulated via post-translational modifications (e.g. phosphorylation, acetylation, ubiquitination and methylation) and is responsive to activating stress signaling and inhibitory insulin (or growth factor)/PI3K/AKT signaling.

Forkhead box protein O1 (FOXO1), also known as forkhead in rhabdomyosarcoma (FKHR), is encoded by the Forkhead Box O1 gene. FOXO1 is a key player in the regulation of gluconeogenesis and glycogenolysis by insulin signaling and is involved in adipocyte differentiation. Additionally, selective deletion or inhibition of FOXO1 in enteroendocrine progenitor cells in the gastrointestinal tract converts the cells into glucose-responsive insulin-producing cells.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In some embodiments, the present disclosure provides a compound of the formula (Ib):

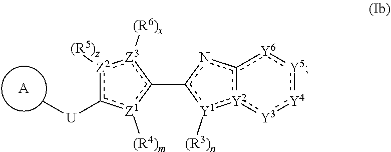

wherein:
A is an aryl or heteroaryl of the formula:

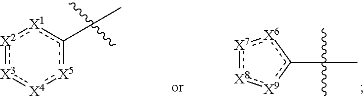

each [symbol] is independently a single bond or a double bond;
each of X¹, X², X³, X⁴, X⁵, X⁶, X⁷, X⁸, and X⁹ is independently CR¹, N, NR¹, O, or S;
Y¹ is C, N, or O wherein, (i) when Y¹ is O, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, or O; wherein (i) when Z¹ is O, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is —C(═O)—N(R^{7a})—, wherein R^{7a} is hydrogen or C₁-C₆ alkyl;
each R¹ is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen, wherein

when A is phenyl or 4-pyridyl,
(i) at least one of R¹ is independently Br, CN, —SCH₃, —S(O)₂CH₃, —O(CH₂)₂OCH₃, —O(CH₂)₃OCH₃, —O(CH₂)₂morpholinyl, —OCH₂COOH, —OCH₂C(═O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OH, —NHC(CH₃)—CH₂OCH₃, —SCH₂CH₂OCH₃; or (ii) at least one of R¹ is NR⁹R¹⁰, wherein NR⁹R¹⁰ is piperidinyl, piperazinyl or pyrrolidinyl, each of which is substituted by —(CH₂)ₜOR¹⁹, wherein R¹⁹ is hydrogen, C₁-C₆ alkyl, or N-, O-, or S-containing heterocyclyl, and t is 0, 1, 2, 3, 4, 5, or 6;
each R² is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R^{13,} R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof.

In some embodiments, the present disclosure provides a compound of formula (Ia):

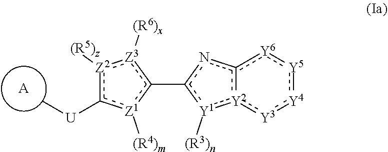

wherein:
A is

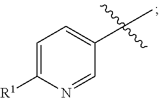

Y¹ is C, N, or O wherein, (i) when Y¹ is O, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, or O; wherein (i) when Z¹ is O, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is a bond or a linker moiety;
each R¹ is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R² is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof.

In some embodiments, the present disclosure provides a compound of formula (I):

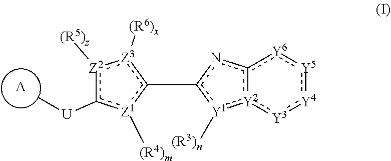

wherein:
A is an aryl or heteroaryl of the formula:

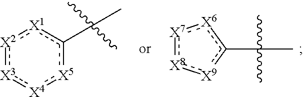

each [symbol] is independently a single bond or a double bond;
each of X¹, X², X³, X⁴, X⁵, X⁶, X⁷, X⁸, and X⁹ is independently CR¹, N, NR¹, O, or S;
Y¹ is C, N, or O wherein, (i) when Y¹ is O, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, or O; wherein (i) when Z¹ is O, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is —C(═O)—N(R^{7a})—, wherein R^{7a} is hydrogen or C₁—C₆ alkyl;
each R¹ is independently alkyl, alkenyl, alkynyl, cycloalkyl, aryl, arylalkyl, alkylaryl, heterocyclyl, heteroaryl —OR⁷, —CN, —NH₂, —NHR⁸, —NR₉R₁₀, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R² is independently hydrogen, halogen or C₁-C₆ alkoxy;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof,
   provided that:

when Z¹ is C, Z² and Z³ are each N; Y¹ is N, Y² is C; R^{7a} is hydrogen or C₁—C₃ alkyl; A is phenyl or 4-pyridyl; and R¹ is hydrogen, C₁—C₃ alkoxy, chlorine (Cl), fluorine (F), C₁—C₆ alkyl, —CF₃, —OH, amine, alkylamine, amide, or heterocyclic amine;

then compounds of formula (I) wherein

(i) R² is hydrogen, Cl, F, or C₁—C₃ alkoxy;

(ii) R³ is hydrogen or C₁-C₃ alkyl;

(iii) R⁴ is hydrogen;

(iv) R⁵, if present, is hydrogen or C₁-C₃ alkyl; and

(v) R⁶, if present, is hydrogen or C₁-C₃ alkyl;

are excluded.

In some embodiments, the present disclosure provides a compound of formula (Ic):

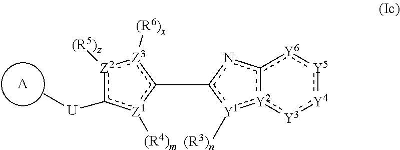

wherein:
A is an aryl or heteroaryl of the formula:

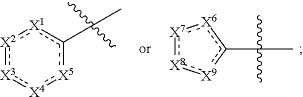

each [symbol] is independently a single bond or a double bond;
each of X¹, X², X³, X⁴, X⁵, X⁶, X⁷, X⁸, and X⁹ is independently CR¹, N, NR¹, O, or S;
Y¹ is C, N, S, or O wherein, (i) when Y¹ is O or S, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, S, or O; wherein (i) when Z¹ is O or S, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is —C(═O)—N(R^{7a})—, wherein R^{7a} is hydrogen or C₁-C₆ alkyl;
each R¹ is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R² is independently hydrogen, halogen or C₁-C₆ alkoxy;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof,
   provided that:

when Z¹ is C, Z² and Z³ are each N; Y¹ is N, Y² is C; A is phenyl or 4-pyridyl; and R¹ is hydrogen, C₁-C₃ alkoxy, chlorine (Cl), fluorine (F), C₁-C₆ alkyl, —CF₃, —OH, amine, alkylamine, amide, or heterocyclic amine;

then compounds of formula (I) wherein

(i) R² is hydrogen, Cl, F, or C₁-C₃ alkoxy;

(ii) R³ is hydrogen or C₁-C₃ alkyl;

(iii) R⁴ is hydrogen;

(iv) R⁵, if present, is hydrogen or C₁-C₃ alkyl; and

(v) R⁶, if present, is hydrogen or C₁-C₃ alkyl;

are excluded.

In some embodiments, the present disclosure provides a compound of formula (Ic′)

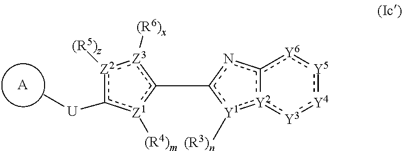

wherein:
A is

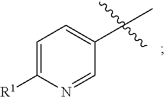

Y¹ is C, N, S, or O wherein, (i) when Y¹ is O or S, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, S, or O; wherein (i) when Z¹ is O or S, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is a bond or a linker moiety;
each R¹ is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R² is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof.

In some embodiments, the present disclosure provides a compound of formula (Ic″):

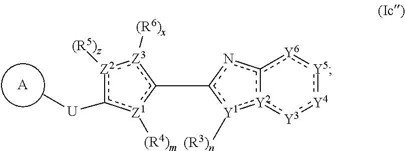

wherein:
A is an aryl or heteroaryl of the formula:

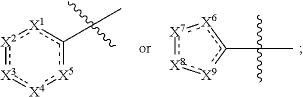

each [symbol] is independently a single bond or a double bond;
each of X¹, X², X³, X⁴, X⁵, X⁶, X⁷, X⁸, and X⁹ is independently CR¹, N, NR¹, O, or S;
Y¹ is C, N, S, or O wherein, (i) when Y¹ is O or S, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, S, or O; wherein (i) when Z¹ is O or S, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is —C(═O)—N(R^{7a})—, wherein R^{7a} is hydrogen or C₁-C₆ alkyl;
each R¹ is independently alkyl, alkenyl, alkynyl, cycloalkyl, aryl, arylalkyl, alkylaryl, heterocyclyl, heteroaryl —OR⁷, —CN, —NH², —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen, wherein

when A is phenyl or 4-pyridyl,
(i) at least one of R¹ is independently Br, CN, —SCH₃, —S(O)₂CH₃, —O(CH₂)₂OCH₃, —O(CH₂)₃OCH₃, —O(CH₂)₂morpholinyl, —OCH₂COOH, —OCH₂C(═O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OH, —NHC(CH₃)—CH₂OCH₃, —SCH₂CH₂OCH₃; or
(ii) at least one of R¹ is NR⁹R¹⁰, wherein NR⁹R¹⁰ is piperidinyl, piperazinyl or pyrrolidinyl, each of which is substituted by —(CH₂)ₜOR¹⁹, wherein R¹⁹ is hydrogen, C₁-C₆ alkyl, or N-, O-, or S-containing heterocyclyl, and t is 0, 1, 2, 3, 4, 5, or 6;
each R² is independently alkyl, alkenyl, alkynyl, cycloalkyl, aryl, arylalkyl, alkylaryl, heterocyclyl, heteroaryl, —OR⁷, —CN, —NH², —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R^{15,} and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof.

In some embodiments, the present disclosure provides a compound which is represented by the structure of formula (VIa):

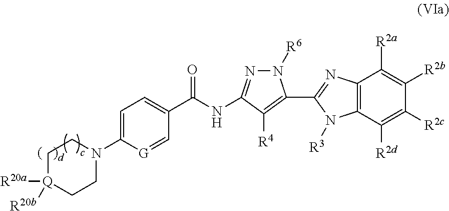

wherein
G is N or CH;
Q is N or C;
each R^{2a}, R^{2b}, R^{2c} and R^{2d} is independently alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, (CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl;
each R^{20a} and R^{20b} is independently:
i) —(CH₂)ₜOR¹⁹, wherein R¹⁹ is hydrogen, a C₁-C₆ alkyl, wherein the C₁-C₆ alkyl is unsubstituted or substituted, or a N, O, or S-containing heterocyclyl;
ii) —C(O)R¹⁹a, wherein R^{19a} is C₁-C₆ alkyl or —(CH₂)ₓOR²¹, wherein R²¹ is C₁-C₆ alkyl that is unsubstituted or substituted with NH₂ or OH, wherein x is 0 or 1;
iii) —NR²²R²³, wherein R²² and R²³ are independently C₁-C₆ alkyl, or R²² and R²³, together with the nitrogen to which are R²² and R²³ are attached, form an unsubstituted or substituted heterocyclic ring;
iv) —OR²⁴, wherein R²⁴ is C₁-C₆ alkyl;
v) an oxygen-containing heterocyclic ring;
vi) —C₁-C₆ alkyl, —OH, or absent; or
vii) hydrogen, provided that at least one of R^{20a} and R^{20b} is not hydrogen; or R^{20a} and R^{20b} together with Q form an oxygen-containing heterocyclic ring;
c is 0 or 1;
d is 0 or 1;
each q is independently 1, 2, 3, 4, 5, or 6, and
t is 0, 1, 2, 3, 4, 5 or 6;
   or a pharmaceutically-acceptable salt or a tautomer thereof.

In some embodiments, the present disclosure provides a compound of formula (VIb′):

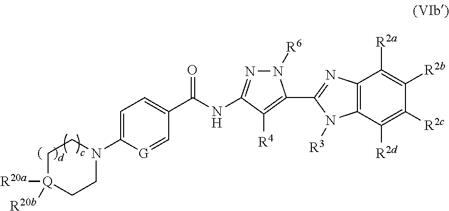

wherein
G is N or CH;
Q is N or C;
Each R^{2a}, R^{2b}, R^{2c} and R^{2d} is independently hydrogen, halogen or C₁-C₆ alkoxy;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R¹⁷ is independently hydrogen or alkyl;
each R^{20a} and R^{20b} is independently:
i) —(CH₂)ₜOR¹⁹, wherein R¹⁹ is hydrogen, a C₁-C₆ alkyl, wherein the C₁-C₆ alkyl is unsubstituted or substituted, or a N, O, or S-containing heterocyclyl;
ii) —C(O)R^{19a}, wherein R¹⁹ is C₁-C₆ alkyl or —(CH₂)ₓOR²¹, wherein R²¹ is C₁-C₆ alkyl that is unsubstituted or substituted with NH₂ or OH, wherein x is 0 or 1;
iii) —NR²²R²³, wherein R²² and R²³ are independently C₁-C₆ alkyl, or R²² and R²³, together with the nitrogen to which are R²² and R²³ are attached, form an unsubstituted or substituted heterocyclic ring;
iv) —OR²⁴, wherein R²⁴ is C₁-C₆ alkyl;
v) an oxygen-containing heterocyclic ring;
vi) —C₁-C₆ alkyl, —OH, or absent; or
vii) hydrogen, provided that at least one of R^{20a} and R^{20b} is not hydrogen; or R^{20a} and R^{20b} together with Q form an oxygen-containing heterocyclic ring;
c is 0 or 1;
d is 0 or 1;
each q is independently 1, 2, 3, 4, 5, or 6; and
t is 0, 1, 2, 3, 4, 5 or 6;
   or a pharmaceutically-acceptable salt or a tautomer thereof.

In some embodiments, the present disclosure provides a compound that is N-[5-(1H-benzimidazol-2-yl)-1-methyl-pyrazol-3-yl]-4-morpholino-benzamide, or a pharmaceutically-acceptable salt or a tautomer thereof. In some embodiments, the present disclosure provides a compound that is N-[5-(1H-benzimidazol-2-yl)-1-methyl-pyrazol-3-yl]-6-[4-(hydroxymethyl)-1-piperidyl]pyridine-3-carboxamide, or a pharmaceutically-acceptable salt or a tautomer thereof. In some embodiments, the present disclosure provides a compound that is N-[5-(1H-benzimidazol-2-yl)-1H-pyrazol-3-yl]-4-methylsulfonyl-benzamide, or a pharmaceutically-acceptable salt or a tautomer thereof. In some embodiments, the present disclosure provides a compound that is N-[5-(1H-benzimidazol-2-yl)-1-methyl-pyrazol-3-yl]-6-(3-methoxyazetidin-1-yl)pyridine-3-carboxamide, or a pharmaceutically-acceptable salt or a tautomer thereof.

In some embodiments, the present disclosure provides a compound that is N-[5-(1H-benzimidazol-2-yl)-1-[(4-methoxyphenyl)methyl]pyrazol-3-yl]-3-chloro-4-methoxy-benzamide, or a pharmaceutically-acceptable salt or a tautomer thereof. In some embodiments, the present disclosure provides a compound that is N-[5-(1H-benzimidazol-2-yl)-1H-pyrazol-3-yl]-6-(1-oxa-8-azaspiro[4.5]decan-8-yl)pyridine-3-carboxamide, or a pharmaceutically-acceptable salt or a tautomer thereof, or a pharmaceutically-acceptable salt or a tautomer thereof. In some embodiments, the present disclosure provides a compound that is 6-[4-(dimethylamino)-1-piperidyl]-N-[5-(5-fluoro-1H-benzimidazol-2-yl)-1-methyl-pyrazol-3-yl]pyridine-3-carboxamide, or a pharmaceutically-acceptable salt or a tautomer thereof. In some embodiments, the present disclosure provides a compound that is N-[5-(1H-benzimidazol-2-yl)-1H-pyrazol-3-yl]-6-[4-(hydroxymethyl)-1-piperidyl]pyridine-3-carboxamide, or a pharmaceutically-acceptable salt or a tautomer thereof.

In some embodiments, the present disclosure provides a compound that is N-[5-(1H-1,3-benzodiazol-2-yl)-1H-pyrazol-3-yl]-4-[4-(2-hydroxyethyl)piperazin-1-yl]benzamide, or a pharmaceutically-acceptable salt or a tautomer thereof. In some embodiments, the present disclosure provides a compound that is N-[5-(1H-benzimidazol-2-yl)-1-methyl-pyrazol-3-yl]-6-[4-(oxetan-3-yl)piperazin-1-yl]pyridine-3-carboxamide, or a pharmaceutically-acceptable salt or a tautomer thereof. In some embodiments, the present disclosure provides a compound that is N-[5-(5-chloro-1H-benzimidazol-2-yl)-1H-pyrazol-3-yl]-6-(4-hydroxy-1-piperidyl)pyridine-3-carboxamide, or a pharmaceutically-acceptable salt or a tautomer thereof. In some embodiments, the present disclosure provides a compound that is N-[5-(1H-benzimidazol-2-yl)-1-methyl-pyrazol-3-yl]-6-(4-methylpiperazin-1-yl)pyridine-3-carboxamide, or a pharmaceutically-acceptable salt or a tautomer thereof.

In some embodiments, the present disclosure provides a compound that is N-[5-(1H-benzimidazol-2-yl)-1H-pyrazol-3-yl]-4-[(3R)-3-hydroxypyrrolidin-1-yl]benzamide , or a pharmaceutically-acceptable salt or a tautomer thereof. In some embodiments, the present disclosure provides a compound that is N-[5-(1H-benzimidazol-2-yl)-1-methyl-pyrazol-3-yl]-4-[4-(oxetan-3-yl)piperazin-1-yl]benzamide, or a pharmaceutically-acceptable salt or a tautomer thereof. In some embodiments, the present disclosure provides a compound that is N-[5-(1H-benzimidazol-2-yl)-1H-pyrazol-3-yl]-4-[4-(oxetan-3-yl)piperazin-1-yl]benzamide, or a pharmaceutically-acceptable salt or a tautomer thereof. In some embodiments, the present disclosure provides a compound that is N-[5-(1H-benzimidazol-2-yl)-1H-pyrazol-3-yl]-3-cyano-4-(2-hydroxyethoxy)benzamide, or a pharmaceutically-acceptable salt or a tautomer thereof. In some embodiments, the present disclosure provides a compound that is N-[5-(5-fluoro-1H-benzimidazol-2-yl)-1-methyl-pyrazol-3-yl]-6-(4-methoxy-1-piperidyl)pyridine-3-carboxamide, or a pharmaceutically-acceptable salt or a tautomer thereof. In some embodiments, the present disclosure provides a compound that is N-[5-(1H-benzimidazol-2-yl)-1-methyl-pyrazol-3-yl]-4-(2-hydroxyethoxy)-3-(trifluoromethyl)benzamide, or a pharmaceutically-acceptable salt or a tautomer thereof.

In some embodiments, the present disclosure provides a pharmaceutical composition comprising a compound of the disclosure and a pharmaceutically-acceptable excipient in a unit dosage form.

In some embodiments, the present disclosure provides a method of treating a condition, the method comprising administering to a subject in need thereof a therapeutically-effective amount of a compound of the disclosure. In some embodiments, the present disclosure provides a method of treating a condition, the method comprising administering to a subject in need thereof a therapeutically-effective amount of a pharmaceutical composition of the disclosure. In some embodiments, the condition is diabetes.

### DETAILED DESCRIPTION

The present disclosure provides a compound that includes a structure that inhibits the transcription factor Forkhead Box O1 (FOXO1). Small molecule effectors of FOXO1 can be useful for the modulation of adipogenesis, gluconeogenesis, glycogenolysis, inflammation, cell proliferation, and stress response, and treatment of disorders that exhibit dysregulation of such processes. Selective inhibition of FOXO1 in the gastrointestinal tract can convert enteroendocrine cells into glucose-dependent insulin-producing cells, thereby providing an endogenous source of insulin to replace pancreatic beta cell function and treat insulin-dependent diabetes. In some embodiments, the disclosure provides a method of treating a condition, the method comprising administering to a subject in need thereof a therapeutically-effective amount of any compound described herein. In some embodiments, the condition is a metabolic disorder. In some embodiments, the condition is diabetes.

### Compounds of the Disclosure.

In some embodiments, the present disclosure provides a compound of formula (Ib)

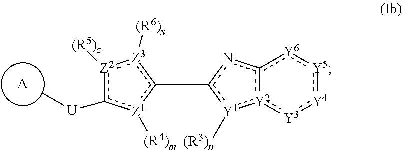

wherein:
A is an aryl or heteroaryl of the formula:

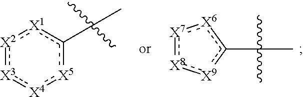

each [symbol] is independently a single bond or a double bond;
each of X¹, X², X³, X⁴, X⁵, X⁶, X⁷, X⁸, and X⁹ is independently CR¹, N, NR¹, O, or S;
Y¹ is C, N, or O wherein, (i) when Y¹ is O, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, or O; wherein (i) when Z¹ is O, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is —C(═O)—N(R^{7a})—, wherein R^{7a} is hydrogen or C₁-C₆ alkyl;
each R¹ is independently alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen, each R² is independently alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof.

In some embodiments, the present disclosure provides a compound of the formula (Ib):

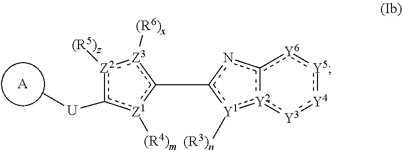

wherein:
A is an aryl or heteroaryl of the formula:

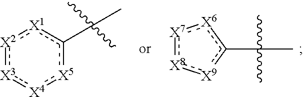

each [symbol] is independently a single bond or a double bond;
each of X¹, X², X³, X⁴, X⁵, X⁶, X⁷, X⁸, and X⁹ is independently CR¹, N, NR¹, O, or S;
Y¹ is C, N, or O wherein, (i) when Y¹ is 0, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, or O; wherein (i) when Z¹ is O, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is —C(═O)—N(R^{7a})—, wherein R^{7a} is hydrogen or C₁-C₆ alkyl;
each R¹ is independently alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen, wherein
when A is phenyl or 4-pyridyl,
   (i) at least one of R¹ is independently Br, CN, —SCH₃, —S(O)₂CH₃, —O(CH₂)₂OCH₃, —O(CH₂)₃OCH₃, —O(CH₂)₂morpholinyl, —OCH₂COOH, —OCH₂C(═O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OH, —NHC(CH₃)—CH₂OCH₃, —SCH₂CH₂OCH₃; or
   (ii) at least one of R¹ is NR⁹R¹⁰, wherein NR⁹R¹⁰ is piperidinyl, piperazinyl or pyrrolidinyl, each of which is substituted by —(CH₂)ₜOR¹⁹, wherein R¹⁹ is hydrogen, C₁-C₆ alkyl, or N-, O-, or S-containing heterocyclyl, and t is 0, 1, 2, 3, 4, 5, or 6;
each R² is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof.

In some embodiments, the present disclosure provides a compound by the structure of formula (VIa):

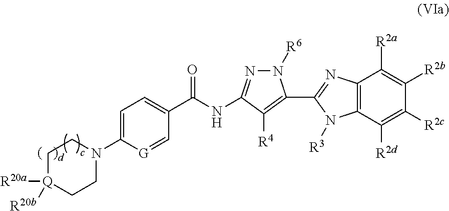

wherein
G is N or CH;
Q is N or C;
each R^{2a}, R^{2b}, R^{2c} and R^{2d} is independently alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl;
each R^{20a} and R^{20b} is independently:
i) —(CH₂)ₜOR¹⁹, wherein R¹⁹ is hydrogen, a C₁-C₆ alkyl, wherein the C₁-C₆ alkyl is unsubstituted or substituted, or a N, O, or S-containing heterocyclyl;
ii) —C(O)R^{19a}, wherein R^{19a} is C₁-C₆ alkyl or —(CH₂)ₓOR²¹, wherein R²¹ is C₁-C₆ alkyl that is unsubstituted or substituted with NH₂ or OH, wherein x is 0 or 1;
iii) —NR²²R²³, wherein R²² and R²³ are independently C₁-C₆ alkyl, or R²² and R²³, together with the nitrogen to which are R²² and R²³ are attached, form an unsubstituted or substituted heterocyclic ring;
iv) —OR²⁴, wherein R²⁴ is C₁-C₆ alkyl;
v) an oxygen-containing heterocyclic ring;
vi) —C₁-C₆ alkyl, —OH, or absent; or
vii) hydrogen, provided that at least one of R^{20a} and R^{20b} is not hydrogen; or R^{20a} and R^{20b} together with Q form an oxygen-containing heterocyclic ring;
c is 0 or 1;
d is 0 or 1;
each q is independently 1, 2, 3, 4, 5, or 6, and
t is 0, 1,2,3,4, 5 or 6;
   or a pharmaceutically-acceptable salt or a tautomer thereof.

In some embodiments, the present disclosure provides a compound of formula (Ia):

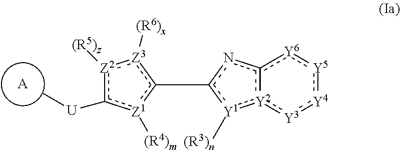

wherein:
A is

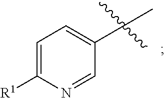

Y¹ is C, N, or O wherein, (i) when Y¹ is O, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, or O; wherein (i) when Z¹ is O, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is a bond or a linker moiety;
each R¹ is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R² is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof.

In some embodiments, the present disclosure provides a compound of formula (I):

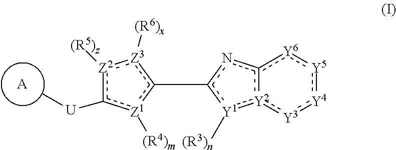

wherein:
A is an aryl or heteroaryl of the formula:

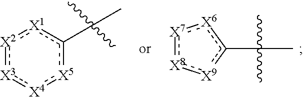

each [symbol] is independently a single bond or a double bond;
each of X¹, X², X³, X⁴, X⁵, X⁶, X⁷, X⁸, and X⁹ is independently CR¹, N, O, or S;
Y¹ is C, N, or O wherein, (i) when Y¹ is 0, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, or O; wherein (i) when Z¹ is O, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is —C(═O)—N(R^{7a})—, wherein R^{7a} is hydrogen or C₁-C₆ alkyl;
each R¹ is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R² is independently hydrogen, halogen or C₁-C₆ alkoxy;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof.

In some embodiments, the present disclosure provides a compound of formula (I):

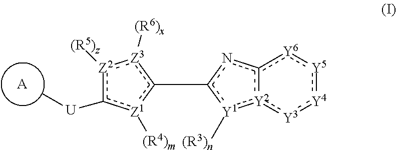

wherein:
A is an aryl or heteroaryl of the formula:

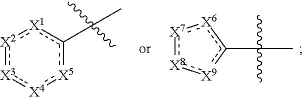

each [symbol] is independently a single bond or a double bond;
each of X¹, X², X³, X⁴, X⁵, X⁶, X⁷, X⁸, and X⁹ is independently CR¹, N, NR¹, O, or S;
Y¹ is C, N, or O wherein, (i) when Y¹ is 0, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, or O; wherein (i) when Z¹ is O, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is —C(═O)—N(R^{7a})—, wherein R^{7a} is hydrogen or C₁-C₆ alkyl;
each R¹ is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R² is independently hydrogen, halogen or C₁-C₆ alkoxy;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof,
   provided that:

when Z¹ is C, Z² and Z³ are each N; Y¹ is N, Y² is C; R^{7a} is hydrogen or C₁-C₃ alkyl; A is phenyl or 4-pyridyl; and R¹ is hydrogen, C₁-C₃ alkoxy, chlorine (Cl), fluorine (F), C₁-C₆ alkyl, —CF₃, —OH, amine, alkylamine, amide, or heterocyclic amine;

then compounds of formula (I) wherein

(i) R² is hydrogen, Cl, F, or C₁-C₃ alkoxy;

(ii) R³ is hydrogen or C₁-C₃ alkyl;

(iii) R⁴ is hydrogen;

(iv) R⁵, if present, is hydrogen or C₁-C₃ alkyl; and

(v) R⁶, if present, is hydrogen or C₁-C₃ alkyl;

are excluded.

In some embodiments, the present disclosure provide a compound of formula (Ic):

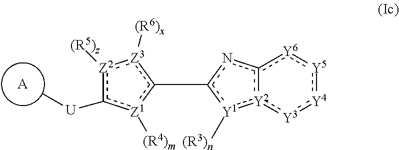

wherein:
A is an aryl or heteroaryl of the formula:

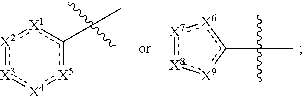

each [symbol] is independently a single bond or a double bond;
each of X¹, X², X³, X⁴, X⁵, X⁶, X⁷, X⁸, and X⁹ is independently CR¹, N, O, or S;
Y¹ is C, N, S, or O wherein, (i) when Y¹ is 0, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, S, or O; wherein (i) when Z¹ is O or S, m is 0, (ii) when Z¹ is N, m is 0 or 1, and (iii) wherein Z¹ is C, m is 1;
Z² is C, N, or O; wherein (i) when Z² is O or S, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is —C(═O)—N(R^{7a})—, wherein R^{7a} is hydrogen or C₁-C₆ alkyl;
each R¹ is independently alkyl, alkenyl, alkynyl, cycloalkyl, aryl, arylalkyl, alkylaryl, heterocyclyl, heteroaryl —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R² is independently hydrogen, halogen or C₁-C₆ alkoxy;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R^{15,} and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof.

In some embodiments, the present disclosure provides a compound of formula (Ic):

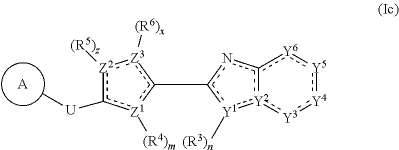

wherein:
A is an aryl or heteroaryl of the formula:

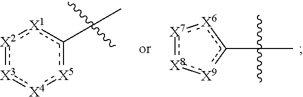

each [symbol] is independently a single bond or a double bond;
each of X¹, X², X³, X⁴, X⁵, X⁶, X⁷, X⁸, and X⁹ is independently CR¹, N, NR¹, O, or S;
Y¹ is C, N, S, or O wherein, (i) when Y¹ is O or S, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, S, or O; wherein (i) when Z¹ is O or S, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is —C(═O)—N(R^{7a})—, wherein R^{7a} is hydrogen or C₁-C₆ alkyl;
each R¹ is independently alkyl, alkenyl, alkynyl, cycloalkyl, aryl, arylalkyl, alkylaryl, heterocyclyl, heteroaryl —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R² is independently hydrogen, halogen or C₁-C₆ alkoxy;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R^{15,} and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof,
   provided that:

when Z¹ is C, Z² and Z³ are each N; Y¹ is N, Y² is C; A is phenyl or 4-pyridyl; and R¹ is hydrogen, C₁-C₃ alkoxy, chlorine (Cl), fluorine (F), C₁-C₆ alkyl, —CF₃, —OH, amine, alkylamine, amide, or heterocyclic amine;

then compounds of formula (I) wherein

(i) R² is hydrogen, Cl, F, or C₁-C₃ alkoxy;

(ii) R³ is hydrogen or C₁-C₃ alkyl;

(iii) R⁴ is hydrogen;

(iv) R⁵, if present, is hydrogen or C₁-C₃ alkyl; and

(v) R⁶, if present, is hydrogen or C₁-C₃ alkyl;

are excluded.

In some embodiments, the present disclosure provides a compound of formula (Ic′)

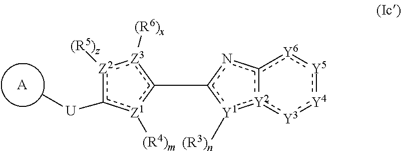

wherein:
A is

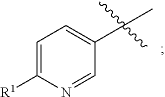

Y¹ is C, N, S, or O wherein, (i) when Y¹ is O or S, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, S, or O; wherein (i) when Z¹ is O or S, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is a bond or a linker moiety;
each R¹ is independently alkyl, alkenyl, alkynyl, cycloalkyl, aryl, arylalkyl, alkylaryl, heterocyclyl, heteroaryl —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R² is independently alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof.

In some embodiments, the present disclosure provides a compound of formula (Ic″):

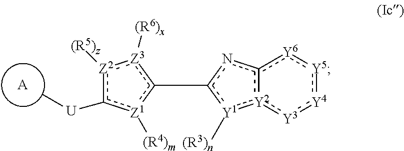

wherein:
A is an aryl or heteroaryl of the formula:

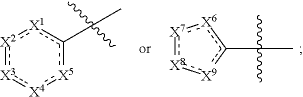

each [symbol] is independently a single bond or a double bond;
each of X¹, X², X³, X⁴, X⁵, X⁶, X⁷, X⁸, and X⁹ is independently CR¹, N, NR¹, O, or S;
Y¹ is C, N, S, or O wherein, (i) when Y¹ is 0, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, S, or O; wherein (i) when Z¹ is O or S, m is 0, (ii) when Z¹ is N, m is 0 or 1, and (iii) wherein Z¹ is C, m is 1;
Z² is C, N, or O; wherein (i) when Z² is O or S, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is —C(═O)—N(R^{7a})—, wherein R^{7a} is hydrogen or C₁-C₆ alkyl;
each R¹ is independently alkyl, alkenyl, alkynyl, cycloalkyl, aryl, arylalkyl, alkylaryl, heterocyclyl, heteroaryl —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen, wherein
when A is phenyl or 4-pyridyl,
   (i) at least one of R¹ is independently Br, CN, —SCH₃, —S(O)₂CH₃, —O(CH₂)₂OCH₃, —O(CH₂)₃OCH₃, —O(CH₂)₂morpholinyl, —OCH₂COOH, —OCH₂C(═O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OH, —NHC(CH₃)—CH₂OCH₃, —SCH₂CH₂OCH₃; or
(ii) at least one of R¹ is NR⁹R¹⁰, wherein NR⁹R¹⁰ is piperidinyl, piperazinyl or pyrrolidinyl, each of which is substituted by —(CH₂)ₜOR¹⁹, wherein R¹⁹ is hydrogen, C₁-C₆ alkyl, or N-, O-, or S-containing heterocyclyl, and t is 0, 1, 2, 3, 4, 5, or 6;
each R² is independently alkyl, alkenyl, alkynyl, cycloalkyl, aryl, arylalkyl, alkylaryl, heterocyclyl, heteroaryl —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R^{15,} and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof.

In some embodiments, the present disclosure provides a compound of the formula (I′):

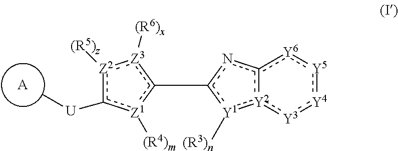

wherein:
A is an aryl or heteroaryl of the formula:

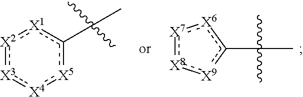

each [symbol] is independently a single bond or a double bond;
each of X¹, X², X³, X⁴, X⁵, X⁶, X⁷, X⁸, and X⁹ is independently CR¹, N, O, or S;
Y¹ is C, N, S, or O wherein, (i) when Y¹ is O or S, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, S, or O; wherein (i) when Z¹ is O or S, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is a bond or a linker moiety;
each R¹ is independently alkyl, alkenyl, alkynyl, cycloalkyl, aryl, arylalkyl, alkylaryl, heterocyclyl, heteroaryl —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R² is independently alkyl, alkenyl, alkynyl, cycloalkyl, aryl, arylalkyl, alkylaryl, heterocyclyl, heteroaryl —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R^{15,} and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof.

In some embodiments, each of X¹, X², X³, X⁴, and X⁵ is independently CR¹, N or NR¹. In some embodiments, each of X¹, X², X³, X⁴, and X⁵ is CR'. In some embodiments, one of X¹, X², X³, X⁴, and X⁵ is N or NR¹ and the others are CR'. In some embodiments, two of X¹, X², X³, X⁴, and X⁵ are N or NR¹ and the others are CR¹.

In some embodiments, compounds of formula (I) wherein A is unsubstituted phenyl are excluded. In some embodiments, compounds of formula (I) wherein A is substituted phenyl are excluded. In some embodiments, compounds of formula (I) wherein A is phenyl substituted with methyl are excluded. In some embodiments, compounds of formula (I) wherein A is phenyl substituted with two methyl groups are excluded. In some embodiments, compounds of formula (I) wherein A is phenyl substituted with halogen are excluded. In some embodiments, compounds of formula (I) wherein A is phenyl substituted with chloro- are excluded. In some embodiments, compounds of formula (I) wherein A is phenyl substituted fluoro- are excluded. In some embodiments, compounds of formula (I) wherein A is phenyl substituted with C₁-C₆ alkyl are excluded. In some embodiments, compounds of formula (I) wherein A is phenyl substituted with trifluoromethyl are excluded. In some embodiments, compounds of formula (I) wherein A is phenyl substituted with —OH are excluded. In some embodiments, compounds of formula (I) wherein A is phenyl substituted with amide are excluded. In some embodiments, compounds of formula (I) wherein A is phenyl substituted with a heterocyclic amine are excluded. In some embodiments, compounds of formula (I) wherein A is phenyl substituted with C₁-C₃ alkoxy are excluded. In some embodiments, compounds of formula (I) wherein A is phenyl substituted with amine are excluded. In some embodiments, compounds of formula (I) wherein A is phenyl substituted with alkylamine are excluded.

In some embodiments, compounds of formula (I) wherein A is unsubstituted 4-pyridyl are excluded. In some embodiments, compounds of formula (I) wherein A is substituted 4-pyridyl are excluded. In some embodiments, compounds of formula (I) wherein A is 4-pyridyl substituted with methyl are excluded. In some embodiments, compounds of formula (I) wherein A is 4-pyridyl substituted with two methyl groups are excluded. In some embodiments, compounds of formula (I) wherein A is 4-pyridyl substituted with halogen are excluded. In some embodiments, compounds of formula (I) wherein A is 4-pyridyl substituted with chloro- are excluded. In some embodiments, compounds of formula (I) wherein A is 4-pyridyl substituted fluoro- are excluded. In some embodiments, compounds of formula (I) wherein A is 4-pyridyl substituted with C₁-C₆ alkyl are excluded. In some embodiments, compounds of formula (I) wherein A is 4-pyridyl substituted with trifluoromethyl are excluded. In some embodiments, compounds of formula (I) wherein A is 4-pyridyl substituted with —OH are excluded. In some embodiments, compounds of formula (I) wherein A is 4-pyridyl substituted with amide are excluded. In some embodiments, compounds of formula (I) wherein A is 4-pyridyl substituted with a heterocyclic amine are excluded. In some embodiments, compounds of formula (I) wherein A is 4-pyridyl substituted with C₁-C₃ alkoxy are excluded. In some embodiments, compounds of formula (I) wherein A is 4-pyridyl substituted with amine are excluded. In some embodiments, compounds of formula (I) wherein A is 4-pyridyl substituted with alkylamine, are excluded.

In some embodiments, when Z¹ is C, Z² and Z³ are each N; Y¹ is N, Y² is C; A is phenyl or 4-pyridyl; U is —R^{a}—N(R)—, —R^{a}—C(═O)—N(R)—, or —C(═O)—N(R)— wherein R is hydrogen or C₁-C₃ alkyl, and R^{a} is C₁-C₆ alkyl; and R¹ is hydrogen, C₁-C₃ alkoxy, chlorine (Cl), fluorine (F), C₁-C₆ alkyl, —CF₃, —OH, amine, alkylamine, amide, or heterocyclic amine; then compounds of formula (I) wherein R² is hydrogen, Cl, F, C₁-C₃ alkoxy, trifluoromethoxy, trifluoromethyl, C₁-C₆ alkyl or C₃-C₁₄ aryl; R³ is hydrogen or C₁-C₃ alkyl; R⁴ is hydrogen; R⁵, if present, is hydrogen or C₁-C₃ alkyl; and R⁶, if present, is hydrogen or C₁-C₃ alkyl, are excluded.

In some embodiments, Z¹ is C; Z² and Z³ are each N; Y¹ is N; Y² is C; U is —C(═O)—N(R)— wherein R is hydrogen or C₁-C₃ alkyl; A is phenyl or 4-pyridyl and R¹ is hydrogen, C₁-C₃ alkoxy, chlorine (Cl), fluorine (F), C₁-C₆ alkyl, —CF₃, —OH, amine, alkylamine, amide, or heterocyclic amine, then compounds of formula (I) wherein (i) R² is hydrogen, Cl, F, C₁-C₃ alkoxy, trifluoromethoxy, trifluoromethyl or C₁-C₆ alkyl or C₃-C₁₄ aryl; (ii) R³ is hydrogen or C₁-C₃ alkyl; (iii) R⁴ is hydrogen; (iv) R⁵, if present, is hydrogen or C₁-C₃ alkyl; and (v) R⁶, if present, is hydrogen or C₁-C₃ alkyl; are excluded.

In some embodiments, when Z¹ is C, Z² and Z³ are each N; Y¹ is N, Y² is C; A is phenyl or 4-pyridyl; U is —R^{a}—N(R)—, —R^{a}—C(═O)—N(R)—, or —C(═O)—N(R)— wherein R is hydrogen or C₁-C₃ alkyl, and R^{a} is C₁-C₆ alkyl; then compounds of formula (I) wherein R² is hydrogen, C₁-C₃ alkoxy, chlorine (Cl), fluorine (F), C₁-C₆ alkyl, —CF₃, —OH, amine, alkylamine, amide, or heterocyclic amine, are excluded.

In some embodiments, when Z¹ is C, Z² and Z³ are each N; Y¹ is N, Y² is C; A is phenyl or 4-pyridyl; U is —R^{a}—N(R)—, —R^{a}—C(═O)—N(R)—, or —C(═O)—N(R)— wherein R is hydrogen or C₁-C₃ alkyl, and R^{a} is C₁-C₆ alkyl; then compounds of formula (I) wherein R² is hydrogen, Cl, F, C₁-C₃ alkoxy, trifluoromethoxy, trifluoromethyl, C₁-C₆ alkyl or C₃-C₁₄ aryl, are excluded.

In some embodiments, when A is phenyl or 4-pyridyl, then at least one R¹ is —CN, —SR¹¹, —S(O)₂R¹², —O(CH₂)_{q}R¹⁴a, —NH(CH₂)_{q}R^{15a}, or —S(CH₂)_{q}R^{16a}, each of which is independently substituted or unsubstituted, wherein R¹⁴a, R¹⁵a, and R¹⁶a are each independently —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted.

In some embodiments, when A is phenyl or 4-pyridyl, then at least one R¹ is a heterocyclic amine that is substituted with hydroxyalkyl, alkenyl, alkynyl, cycloalkyl, aryl, arylalkyl, alkylaryl, heterocyclyl, heteroaryl —OR⁷, —CN, —NH², —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or halogen.

In some embodiments, when A is phenyl or 4-pyridyl, then at least one of R² is heterocyclyl, heteroaryl, —CN, —NH₂, —NHR⁸, —NR⁹R¹⁰, —SR¹¹, —S(O)₂R¹², —NHC(O)R¹³, —O(CH₂)_{q}R¹⁴, NH(CH₂)_{q}R¹⁵, or —S(CH₂)_{q}R¹⁶, each of which is independently substituted or unsubstituted.

In some embodiments, compounds of formula (I) wherein each Y³ and Y⁶ is independently CR² wherein R² is hydrogen, and each Y⁴ and Y⁵ is independently CR² wherein R² is methyl, are excluded.

In some embodiments, the present disclosure provides a compound of the formula (I-A):

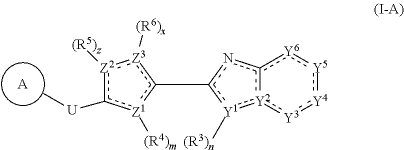

wherein:
A is an aryl or heteroaryl of the formula:

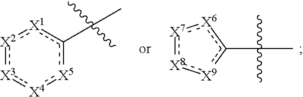

each [symbol] is independently a single bond or a double bond;
each of X¹, X², X³, X⁴, X⁵, X⁶, X⁷, X⁸, and X⁹ is independently CR¹, N, NR¹, O, or S;
Y¹ is C, N, or O wherein, (i) when Y¹ is O, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, or O; wherein (i) when Z¹ is O, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is a bond or a linker moiety;
each R¹ is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R² is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,

or a pharmaceutically-acceptable salt or a tautomer thereof,

provided that:
A) when Z¹ is C, Z² and Z³ are each N; Y¹ is N, Y² is C; A is phenyl or 4-pyridyl; U is —R^{a}—N(R)—, —R^{a}—C(═O)—N(R)—, or —C(═O)—N(R)—, wherein R is hydrogen or C₁-C₃ alkyl, and R^{a} is C₁-C₆ alkyl; R¹ is hydrogen, C₁-C₃ alkoxy, chlorine (Cl), fluorine (F), C₁-C₆ alkyl, —CF₃, —OH, amine, alkylamine, amide, or heterocyclic amine;

then compounds of formula (I) wherein (i) R² is hydrogen, Cl, F, C₁-C₃ alkoxy, trifluoromethoxy, trifluoromethyl or C₁-C₆ alkyl or C₃-C₁₄ aryl; (ii) R³ is hydrogen or C₁-C₃ alkyl; (iii) R⁴ is hydrogen; (iv) R⁵, if present, is hydrogen or C₁-C₃ alkyl; and (v) R⁶, if present, is hydrogen or C₁-C₃ alkyl; are excluded; and
B) compounds wherein each Y³ and Y⁶ is independently CR² wherein R² is hydrogen, and each Y⁴ and Y⁵ is independently CR² wherein R² is methyl, are excluded.

In some embodiments, the present disclosure provides a compound of the formula (I-B):

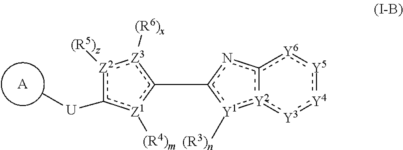

wherein:
A is an aryl or heteroaryl of the formula:

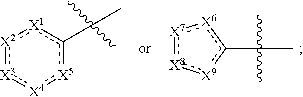

each [symbol] is independently a single bond or a double bond;
each of X¹, X², X³, X⁴, X⁵, X⁶, X⁷, X⁸, and X⁹ is independently CR¹, N, NR¹, O, or S;
Y¹ is C, N, or O wherein, (i) when Y¹ is O, n is 0; (ii) when Y′ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, or O; wherein (i) when Z¹ is O, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is a bond or a linker moiety;
each R¹ is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R² is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,

or a pharmaceutically-acceptable salt or a tautomer thereof,

provided that:

A) when A is phenyl or 4-pyridyl, then

(i) at least one R¹ is:

—CN, —SR¹¹, —S(O)₂R¹², —O(CH₂)_{q}R^{14a}, —NH(CH₂)_{q}R^{15a}, or —S(CH₂)_{q}R^{16a}, each of which is independently substituted or unsubstituted, wherein R^{14a}, R^{15a}, and R^{16a} are each independently —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted; or

a heterocyclic amine that is substituted with hydroxyalkyl, alkenyl, alkynyl, cycloalkyl, aryl, arylalkyl, alkylaryl, heterocyclyl, heteroaryl, —OR⁷, —CN, —NH², —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or halogen; or

(ii) at least one of R² is heterocyclyl, heteroaryl, —OR⁷, —CN, —NH², —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted; and

B) compounds of formula (I) wherein each Y³ and Y⁶ is independently CR² wherein R² is hydrogen, and each Y⁴ and Y⁵ is independently CR² wherein R² is methyl, are excluded.

In some embodiments, A is

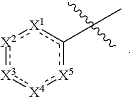

In some embodiments, each of X¹, X², X³, X⁴, and X⁵ is independently CR¹, N or NR¹. In some embodiments, each of X¹, X², X³, X⁴, and X⁵ is CR¹. In some embodiments, one of X¹, X², X³, X⁴, and X⁵ is N or NR¹ and the others are CR¹. In some embodiments, two of X¹, and X⁵ are N or NR¹ and the others are CR¹.

In some embodiments, A is phenyl, pyridyl or pyrimindinyl.

In some embodiments, A is

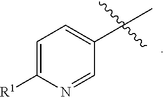

In some embodiments, A is phenyl.

In some embodiments, Y¹ is N and n is 1. In some embodiments, Y² is C. In some embodiments, each of Y³, Y⁴, Y⁵, or Y⁶ is independently CR². In some embodiments, Y¹ is C and Y² is N. In some embodiments, Z¹ is C. In some embodiments, Z² is N and z is 0. In some embodiments, Z³ is N and x is 1. In some embodiments, Y¹ is S. In some embodiments, Z¹ is S.

In some embodiments, U is a bond, alkylene, an amide group, an amino group, a carbamate, a sulfoxide group, a sulfone group, a sulfonamide group, carboxyl group, an ester group, urea, or -L-alkylene-L′- wherein L and L′ are each independently O, N, S; each of which is substituted or unsubstituted.

In some embodiments, U is a bond, alkylene, an amino group, a carbamate, carboxyl group, an ester group, urea, or -L-alkylene-L′- wherein L and L′ are each independently O, N, S; each of which is substituted or unsubstituted.

In some embodiments, U is a bond, alkylene, or -L-alkylene-L′- wherein L and L′ are each independently O, N, S, amide, sulfonamide, or urea.

In some embodiments, U is a bond, —N(R^{7a})—C(═O)—; —C(═O)—N(R^{7a})—, —N(R^{7a})—S(═O)₂—, —S(═O)₂—N(R^{7a})—, —N(R^{7a})—C(═O)N(R^{7b})—, —(CH₂)ₐ—O—, or —O—(CH₂)_{b}—, wherein
each of R^{7a} and R^{7b} is independently alkyl, alkenyl, alkynyl, cycloalkyl, aryl, arylalkyl, alkylaryl, heterocyclyl, or heteroaryl, each of which is independently unsubstituted or substituted, or hydrogen;
each a is independently 0, 1, 2, 3, 4, 5, or 6; and
each b is independently 0, 1, 2, 3, 4, 5, or 6.

In some embodiments, U is —C(═O)—N(R^{7a})—, wherein lea is hydrogen or C₁-C₆ alkyl.

In some embodiments, A is of the formula:

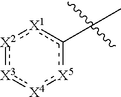

wherein each X¹, X², X³, X⁴ and X⁵ is independently CR¹, N, NR¹, O, or S.

In some embodiments, when any one or more of X¹, X², X³, X⁴, and X⁵ is O, the bonds [symbol] linking said O to the ring is a single bond.

In some embodiments, when any one or more of X¹, X², X³, X⁴, and X⁵ is S, the bonds [symbol] linking said S to the ring are single bond.

In some embodiments, when any one or more of X¹, X², X³, X⁴, and X⁵ is N, the bonds [symbol] linking said N to the ring are a single and a double bond.

In some embodiments, when any one or more of X¹, X², X³, X⁴, and X⁵ is NR¹, the bonds [symbol] linking said NR¹ to the ring are single bonds.

In some embodiments, when any one or more of X¹, X², X³, X⁴, and X⁵ is CR^{O}, the bonds [symbol] linking said NR¹ to the ring are single bonds.

In some embodiments, when any one or more of X¹, X², X³, X⁴, and X⁵ is CR^{O}, the bonds [symbol] linking said NR¹ to the ring are double bonds.

In some embodiments, each of X¹, X², X³, X⁴, and X⁵ is CR¹.

In some embodiments, one of X¹, X², X⁴, and X⁵ is N or NR¹ and the others are CR¹.

In some embodiments, two of X¹, X², X⁴, and X⁵ are N or NR¹ and the others are CR¹.

Non-limiting examples of the substituent A include any of the following formulae:

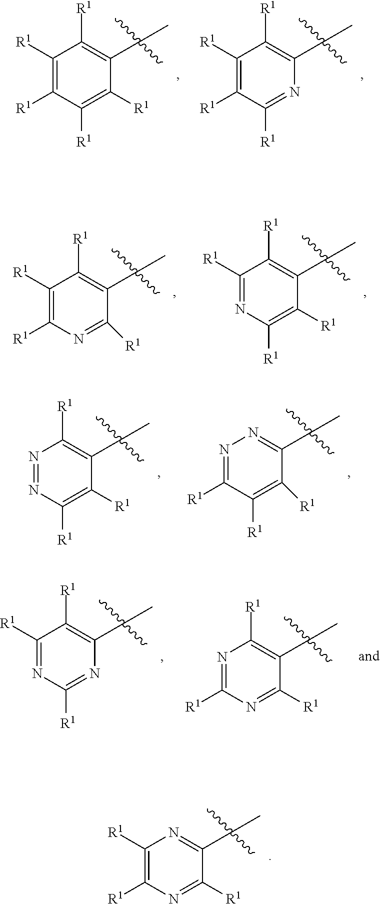

In some embodiments, each R¹ is independently hydrogen, halogen, C₁-C₆ alkyl C₁-C₆ alkoxy, C₁-C₆ haloalkoxy, —OH, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶.

In some embodiments, each R¹ is independently alkyl, alkenyl, alkynyl, cycloalkyl, arylalkyl, alkylaryl, heterocyclyl, heteroaryl, —OR⁷, —CN, —NH², —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen.

In some embodiments, each R¹ is independently hydrogen, Cl, Br, CN, —OCH₃, —SCH₃, —OCF₃, —S(O)₂CH₃, —CH₃, —O(CH₂)₂OCH₃, —O(CH₂)₃OCH₃, —O(CH₂)₂morpholinyl, —OCH₂COOH, —OCH₂C(═O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OH, —NHC(CH₃)—CH₂OCH₃, or —SCH₂CH₂OCH₃, each of which is substituted or unsubstituted.

In some embodiments, each R¹ is independently hydrogen, Br, CN, —SCH₃, —S(O)₂CH₃, —O(CH₂)₂OCH₃, —O(CH₂)₃OCH₃, —O(CH₂)₂morpholinyl, —OCH₂COOH, —OCH₂C(═O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OH, —NHC(CH₃)—CH₂OCH₃, or —SCH₂CH₂OCH₃.

In some embodiments, each R¹ is independently hydrogen, Cl, Br, CN, —OCH₃, —SCH₃, —OCF₃, —S(O)₂CH₃, —CH₃, —O(CH₂)₂OCH₃, —O(CH₂)₃OCH₃, azatadinyl, 2-azaspiro[3.3]heptanyl, 2-oxa-6-azaspiro[3.3]heptanyl, pyrrolidinyl, piperazinyl, piperidinyl, morpholinyl, —O(CH₂)₂morpholinyl, —OC₃H₅O, —OCH₂COOH, —OCH₂C(═O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OH, —NHC(CH₃)—CH₂OCH₃, or —SCH₂CH₂OCH₃, each of which is substituted or unsubstituted.

In some embodiments, an R¹ is NR⁹R¹⁰, wherein R⁹ and R¹⁰ together with the nitrogen atom to which R⁹ and R¹⁰ are attached form an unsubstituted or substituted heterocyclic ring. In some embodiments, NR⁹R¹⁰ is a 4-, 5-, 6-, or 7-membered ring, optionally containing at least one additional heteroatom, wherein the additional heteroatom is O, N or S.

In some embodiments, NR⁹R¹⁰ is piperidinyl, piperazinyl, azetidinyl, pyrrolidinyl, or morpholinyl, each of which is unsubstituted or substituted. In some embodiments, NR⁹R¹⁰ is unsubstituted or substituted piperidinyl. In some embodiments, NR⁹R¹⁰ is unsubstituted or substituted piperazinyl. In some embodiments, NR⁹R¹⁰ is unsubstituted or substituted azetidinyl.

In some embodiments, each piperidinyl, piperazinyl, azetidinyl, pyrrolidinyl, or morpholinyl is independently substituted by —(CH₂)ₜOR¹⁹, wherein R¹⁹ is hydrogen, C₁-C₆ alkyl, or a N-, O-, or S-containing heterocyclyl, and t is 0, 1, 2, 3, 4, 5, or 6.

In some embodiments, each piperidinyl, piperazinyl, azetidinyl, pyrrolidinyl, or morpholinyl is independently substituted by C₁-C₆ alkyl. In some embodiments, each piperidinyl, piperazinyl, azetidinyl, pyrrolidinyl, or morpholinyl is independently substituted —N(R²⁰)₂, wherein each R²⁰ is independently hydrogen or a C₁-C₆ alkyl. In some embodiments, each piperidinyl, piperazinyl, azetidinyl, pyrrolidinyl, or morpholinyl is independently substituted by an oxygen-containing heterocyclic ring. In some embodiments, the oxygen-containing heterocyclic ring is oxetanyl.

In some embodiments, NR⁹R¹⁰ is a bicyclic ring comprising piperidinyl, piperazinyl, azetidinyl, or pyrrolidinyl, each of which is substituted or unsubstituted. In some embodiments, NR⁹R¹⁰ is a fused bicyclic ring. In some embodiments, NR⁹R¹⁰ is pyrazinyl fused to cyclopentanonyl. In some embodiments, NR ⁹R¹⁰ is pyrrolidinyl fused to tetrahydrofuranyl. In some embodiments, NR⁹R¹⁰ is a spiro bicyclic ring moiety. In some embodiments, the bicyclic spiro bicyclic ring moiety is 2-oxa-6-azaspiro[3.4]octanyl, 1-oxa-8-azaspiro[4.5]decanyl, 2-azaspiro-3.3]heptanyl, 2-oxa-6-azaspiro[3.3]heptanyl, or 2-oxa-7-azaspiro[3.5]nonanyl, each of which is substituted or unsubstituted. In some embodiments, NR⁹R¹⁰ is a bridged bicyclic ring moiety. In some embodiments, the bridged bicyclic ring moiety is 3,6-diazabicyclo[3.1.1]heptanyl, 3-oxa-8-azabicycli[3.2.1]octanyl, or 3,8-diazabicyclo[3.2.1]octanyl.

Non-limiting examples of R¹ include any of the following formulae:

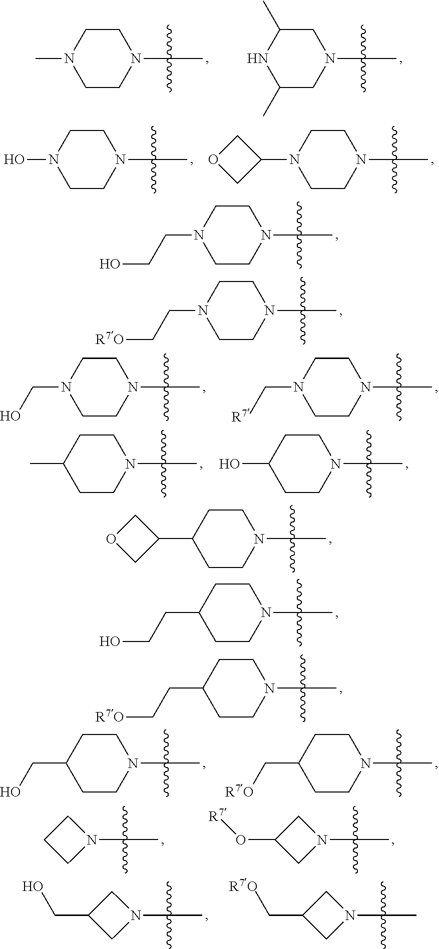

wherein each R^{7′} is independently alkyl, alkenyl, alkynyl, cycloalkyl, aryl, alkylaryl, heterocyclyl, or heteroaryl, each of which is independently unsubstituted or substituted.

Additional non-limiting examples of R¹ include any of the following formulae:

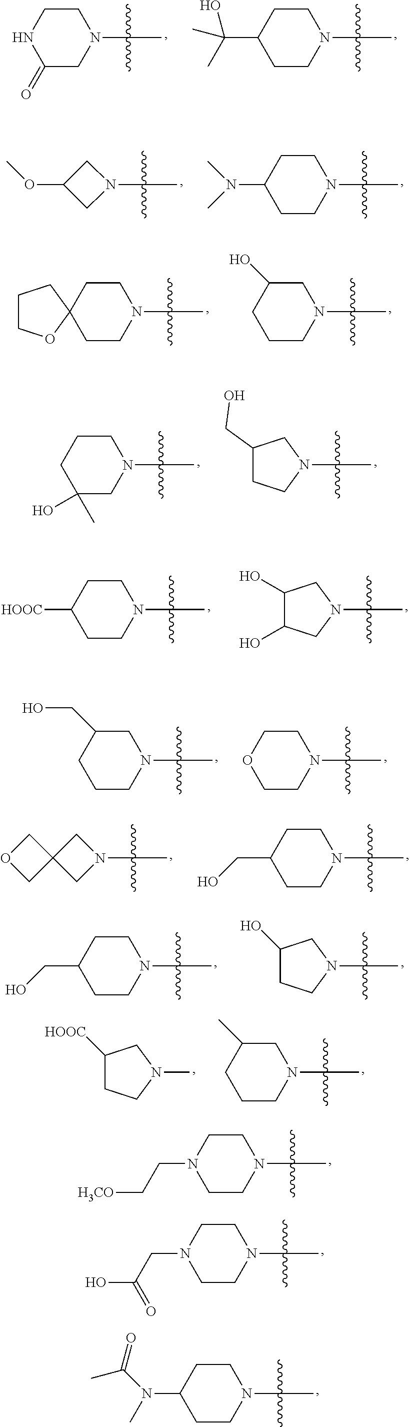

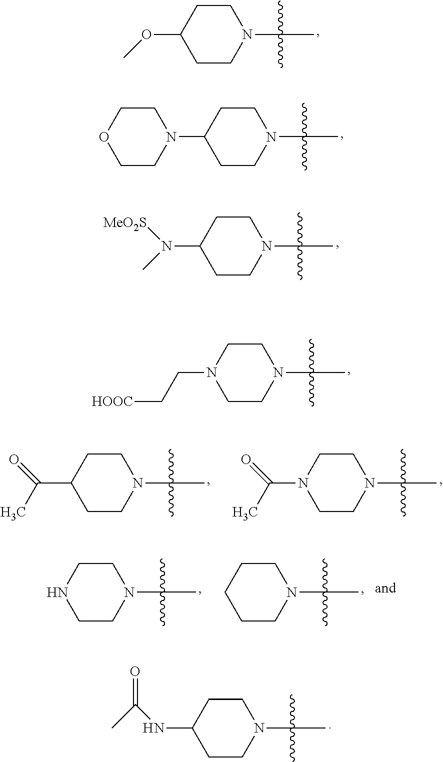

In some embodiments, each R² is C₁-C₆ alkyl, C₁-C₆ haloalkyl, C₁-C₆ alkoxy, or C₁-C₆ haloalkoxy, each or which is unsubstituted or substituted, or hydrogen or halogen. In some embodiments, each R² is independently C₂-C₆ alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen. In some embodiments, each R² is independently hydrogen, halogen or C₁-C₆ alkoxy. In some embodiments, each R² is independently hydrogen, F, Cl, Br, CH₃, CF₃, OCH₃, or OCF₃. In some embodiments, each R² is hydrogen.

In some embodiments, R³ is C₁-C₆ alkyl, —(CH₂)_{q}COOR¹⁷, or —(CH₂)_{q}OR¹⁷, each of which is independently substituted or unsubstituted, or hydrogen or halogen. In some embodiments, R³ is hydrogen. In some embodiments, R³ is —CH₂—COOH.

In some embodiments, R⁴ is C₁-C₆ alkyl, —(CH₂)_{q}COOR¹⁷, or —(CH₂)_{q}OR¹⁷, each of which is independently substituted or unsubstituted, or hydrogen or halogen. In some embodiments, R⁴ is hydrogen.

In some embodiments, z is 0 and x is 1.

In some embodiments, R⁶ is hydrogen, CH₃, —(CH₂)₂OH, or —(CH₂)₂OCH₃. In some embodiments, R⁶ is hydrogen. In some embodiments, R⁶ is CH₃.

In some embodiments, the compound is represented by the structure of formula (II):

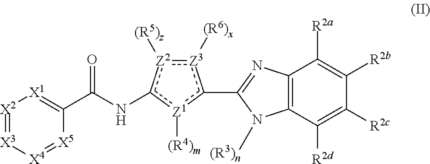

wherein each X¹, X², X³, X⁴, X⁵, Z¹, Z², Z³, R^{2a}, R^{2b}, R^{2c}, R^{3d}, R³, R⁴, R⁵, R⁶, m, n, x, and z are as defined for formula (I), (I-A), (I-B), (Ia), (Ib), (Ic), (Ic′) or (Ic″).

In some embodiments, the compound is represented by the structure of formula (III):

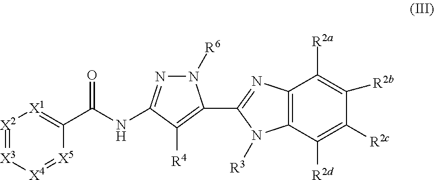

wherein each X¹, X², X³, X⁴, X⁵, R^{2a}, R^{2b}, R^{2c}, R^{2d}, R³, R⁴, R⁵ and R⁶ are as defined for formula (I), (I-A), (I-B), (Ia), (Ib), (Ic), (Ic′) or (Ic″).

In some embodiments, the present disclosure provides a compound of the formula (IV):

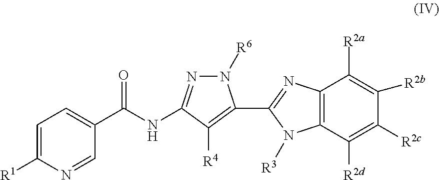

wherein:
each R¹ is independently alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R^{2a}, R^{2b}, R^{2c}, and R^{2d} independently alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, and R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,

or a pharmaceutically-acceptable salt or a tautomer thereof.

In some embodiments, an R¹ in formula (IV) is NR⁹R¹⁰, wherein R⁹ and R¹⁰ together with the nitrogen to which R⁹ and R¹⁰ are attached form an unsubstituted or substituted heterocyclic ring. In some embodiments, NR⁹R¹⁰ is a 4-, 5-, 6-, or 7-membered ring, optionally containing at least one additional heteroatom, wherein the additional heteroatom is O, N or S. In some embodiments, NR⁹R¹⁰ is piperidinyl, piperazinyl, azetidinyl, pyrrolidinyl, or morpholinyl, each of which is unsubstituted or substituted. In some embodiments, NR⁹R¹⁰ is unsubstituted or substituted piperidinyl. In some embodiments, NR⁹R¹⁰ is unsubstituted or substituted piperazinyl. In some embodiments, NR⁹R¹⁰ is unsubstituted or substituted azetidinyl. In some embodiments, each piperidinyl, piperazinyl, azetidinyl, pyrrolidinyl, or morpholinyl is independently substituted by —(CH₂)ₜOR¹⁹, wherein R¹⁹ is hydrogen, C₁-C₆ alkyl, or a N-, O-, or S-containing heterocyclyl, and t is 0, 1, 2, 3, 4, 5, or 6. In some embodiments, each piperidinyl, piperazinyl, azetidinyl, pyrrolidinyl, or morpholinyl is independently substituted by C₁-C₆ alkyl. In some embodiments, each piperidinyl, piperazinyl, azetidinyl, pyrrolidinyl, or morpholinyl is independently substituted —N(R²⁰)₂, wherein each R²⁰ is independently hydrogen or a C₁-C₆ alkyl. In some embodiments, each piperidinyl, piperazinyl, azetidinyl, pyrrolidinyl, or morpholinyl is independently substituted by an oxygen-containing heterocyclic ring. In some embodiments, the oxygen-containing heterocyclic ring is oxetanyl.

In some embodiments, R¹ in formula (IV) is piperidinyl, piperazinyl or pyrrolidinyl, each of which is unsubstituted or substituted. In some embodiments, R¹ in formula (IV) is piperidinyl, piperazinyl or pyrrolidinyl, each of which is substituted by —(CH₂)ₜOR¹⁹, wherein R¹⁹ is hydrogen, C₁-C₆ alkyl, or N-, O-, or S-containing heterocyclyl, and t is 0, 1, 2, 3, 4, 5, or 6.

In some embodiments, R¹ in formula (IV) is piperazinyl. In some embodiments, R¹ in formula (IV) is substituted piperazinyl. In some embodiments, R¹ in formula (IV) is a piperazinyl substituted by —(CH₂)ₜOR¹⁹, wherein R¹⁹ is hydrogen, C₁-C₆ alkyl, or N-, O-, or S-containing heterocyclyl, and t is 0, 1, 2, 3, 4, 5, or 6.

In some embodiments, R¹ in formula (IV) is piperidinyl. In some embodiments, R¹ in formula (IV) is substituted piperidinyl. In some embodiments, R¹ in formula (IV) is a piperidinyl substituted by —(CH₂)ₜOR¹⁹, wherein R¹⁹ is hydrogen, C₁-C₆ alkyl, or N-, O-, or S-containing heterocyclyl, and t is 0, 1, 2, 3, 4, 5, or 6.

In some embodiments, the present disclosure provides a compound of the formula (V):

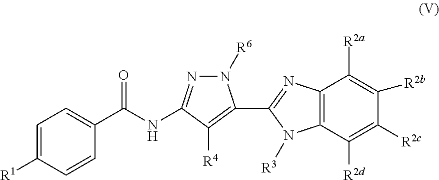

wherein:
each R¹ is independently alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each of R^{2a}, R^{2b}, R^{2c}, and R^{2d} is independently alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R^{15,} and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, (CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,

or a pharmaceutically-acceptable salt or a tautomer thereof.

In some embodiments, compounds of formula (V) wherein R¹ is hydrogen, C₁-C₃ alkoxy, chlorine (Cl), fluorine (F), C₁-C₆ alkyl, —CF₃, —OH, amine, alkylamine, or amide or heterocyclic amine; R^{2a}, R^{2b}, R^{2c}, and R^{2d} is each independently hydrogen, Cl, F, C₁-C₃ alkoxy, trifluoromethoxy, trifluoromethyl, C₁-C₆ alkyl or C₃-C₁₄ aryl; R³ is hydrogen or C₁-C₃ alkyl; R⁴ is hydrogen; and R⁶, is hydrogen or C₁-C₃ alkyl, are excluded.

In some embodiments, compounds of formula (V) wherein R^{2b} and R^{2c} are each methyl, are excluded.

In some embodiments, at least one R¹ is CN, —SR¹¹, —S(O)₂R¹², —O(CH₂)_{q}R^{14a}, —NH(CH₂)_{q}R^{15a}, or —S(CH₂)_{q}R^{16a}, each of which is independently substituted or unsubstituted, wherein R^{14a}, R^{15a}, and R^{16a} are each independently —OH, —OR⁷, —NH₂, —NHR⁸, —NR⁹R¹⁰, (CH₂)_{q}COOR¹⁷, —(CH₂)_{q}N(R¹⁷)₂, —(CH₂)_{q}OR¹⁷, or —COOR¹⁸, each of which is independently unsubstituted or substituted.

In some embodiments, at least one R¹ is a heterocyclic amine that is substituted with hydroxyalkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or halogen.

In some embodiments, R² is heterocyclyl, heteroaryl, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted.

In some embodiments, an R¹ in formula (V) is NR⁹R¹⁰, wherein R⁹ and R¹⁰ together with the nitrogen atom to which R⁹ and R¹⁰ are attached form an unsubstituted or substituted heterocyclic ring. In some embodiments, NR⁹R¹⁰ is a 4-, 5-, 6-, or 7-membered ring, optionally containing at least one additional heteroatom, wherein the additional heteroatom is O, N, or S. In some embodiments, NR⁹R¹⁰ is piperidinyl, piperazinyl, azetidinyl, pyrrolidinyl, or morpholinyl, each of which is unsubstituted or substituted. In some embodiments, NR⁹R¹⁰ is unsubstituted or substituted piperidinyl. In some embodiments, NR⁹R¹⁰ is unsubstituted or substituted piperazinyl. In some embodiments, NR⁹R¹⁰ is unsubstituted or substituted azetidinyl. In some embodiments, each piperidinyl, piperazinyl, azetidinyl, pyrrolidinyl, or morpholinyl is independently substituted by —(CH₂)ₜOR¹⁹, wherein R¹⁹ is hydrogen, C₁-C₆ alkyl, or a N-, O-, or S-containing heterocyclyl, and t is 0, 1, 2, 3, 4, 5, or 6. In some embodiments, each piperidinyl, piperazinyl, azetidinyl, pyrrolidinyl, or morpholinyl is independently substituted by C₁-C₆ alkyl. In some embodiments, each piperidinyl, piperazinyl, azetidinyl, pyrrolidinyl, or morpholinyl is independently substituted —N(R²⁰)₂, wherein each R²⁰ is independently hydrogen or a C₁-C₆ alkyl. In some embodiments, each piperidinyl, piperazinyl, azetidinyl, pyrrolidinyl, or morpholinyl is independently substituted by an oxygen-containing heterocyclic ring. In some embodiments, the oxygen-containing heterocyclic ring is oxetanyl.

In some embodiments, R¹ in formula (V) is piperidinyl, piperazinyl or pyrrolidinyl, each of which is unsubstituted or substituted. In some embodiments, R¹ in formula (V) is piperidinyl, piperazinyl or pyrrolidinyl, each of which is substituted by —(CH₂)ₜOR¹⁹, wherein R¹⁹ is hydrogen, C₁-C₆ alkyl, or N-, O-, or S-containing heterocyclyl, and t is 0, 1, 2, 3, 4, 5, or 6. In some embodiments, R⁶ is methyl and R¹ is piperidinyl, piperazinyl, pyrrolidinyl, or morpholinyl.

In some embodiments, R¹ in formula (V) is piperazinyl. In some embodiments, R¹ in formula (V) is substituted piperazinyl. In some embodiments, R¹ in formula (V) is a piperazinyl substituted by —(CH₂)ₜOR¹⁹, wherein R¹⁹ is hydrogen, C₁-C₆ alkyl, or N-, O-, or S-containing heterocyclyl, and t is 0, 1, 2, 3, 4, 5, or 6.

In other embodiments, R¹ in formula (V) is or piperidinyl. In some embodiments, R¹ in formula (V) substituted piperidinyl. In some embodiments, R¹ in formula (V) is a piperidinyl substituted by —(CH₂)ₜOR¹⁹, wherein R¹⁹ is hydrogen, C₁-C₆ alkyl, or N-, O-, or S-containing heterocyclyl, and t is 0, 1, 2, 3, 4, 5, or 6.

In some embodiments, R⁶ in formula (I), (II), (III), (IV), or (V) is alkylaryl, alkylaryl, —(CH₂)_{q}COOR¹⁷, —(CH₂)_{q}N(R¹⁷)₂, or —(CH₂)_{q}OR¹⁷, each of which is independently substituted or unsubstituted.

In some embodiments, the present disclosure provides a compound of the formula (VI):

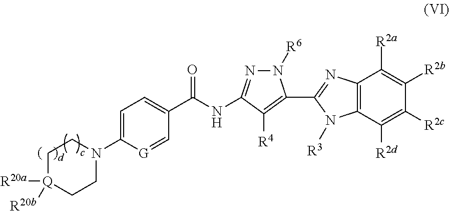

wherein
G is N or CH;
Q is N or C;
each of R^{2a}, R^{2b}, R^{2c}, and R^{2d} is independently alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each R^{20a} and R^{20b} is independently:
i) —(CH₂)ₜOR¹⁹, wherein R¹⁹ is hydrogen, a C₁-C₆ alkyl, wherein the C₁-C₆ alkyl is unsubstituted or substituted, or a N, O, or S-containing heterocyclyl;
ii) —C(O)R^{19a}, wherein R^{19a} is C₁-C₆ alkyl or —(CH₂)ₓOR²¹, wherein R²¹ is C₁-C₆ alkyl that is unsubstituted or substituted with NH₂ or OH, wherein x is 0 or 1;
iii) —NR²²R²³, wherein R²² and R²³ are independently C₁-C₆ alkyl, or R²² and R²³, together with the nitrogen to which are R²² and R²³ are attached, form an unsubstituted or substituted heterocyclic ring;
iv) —OR²⁴, wherein R²⁴ is C₁-C₆ alkyl;
v) an oxygen-containing heterocyclic ring;
vi) —C₁-C₆ alkyl, —OH, or absent; or
vii) hydrogen, provided that at least one of R^{20a} and R^{20b} is not hydrogen; or R^{20a} and R^{20b} together with Q form an oxygen-containing heterocyclic ring;
c is 0 or 1;
d is 0 or 1;
each q is independently 1, 2, 3, 4, 5, or 6; and
t is 0, 1, 2, 3, 4, 5 or 6;
   or a pharmaceutically-acceptable salt or a tautomer thereof.

In some embodiments, the disclosure provides compounds of formula (VI-A):

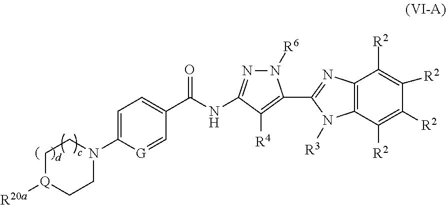

wherein
G is N or CH;
Q is N or C;
each R² is independently alkyl, alkenyl, alkynyl, cycloalkyl, aryl, alkylaryl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, alkylaryl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, alkylaryl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, alkylaryl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, alkylaryl, alkylaryl, heterocyclyl, or heteroaryl, each of which is independently unsubstituted or substituted, or hydrogen;
each R⁸ is independently alkyl, alkenyl, alkynyl, cycloalkyl, aryl, alkylaryl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, alkylaryl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R^{15,} and R¹⁶ is independently alkyl, alkenyl, alkynyl, cycloalkyl, aryl, alkylaryl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently H or alkyl;
R²⁰ is —(CH₂)ₜOR¹⁹, wherein R¹⁹ is hydrogen, a C₁-C₆ alkyl, or a N-, O-, or S-containing heterocyclyl;
c is 0 or 1;
each q is independently 1, 2, 3, 4, 5, or 6; and
t is 0, 1, 2, 3, 4, 5, or 6;

or a pharmaceutically-acceptable salt or a tautomer thereof.

In some embodiments of formula (VI) or (VI-a), R², R^{2a}, R^{2b}, R^{2c}, or R^{2d} is H. In other embodiments of formula (VI) or (VI-a), R², R^{2a}, R^{2b}, R^{2c}, or R^{2d} is OR⁷. In some embodiments of formula (VI) or (VI-a), R², R^{2a}, R^{2b}, R^{2c}, or R^{2d} is halogen, e.g., fluorine.

In some embodiments of formula (VI) or (VI-a), R³ is hydrogen.

In some embodiments of formula (VI) or (VI-a), R⁴ is hydrogen

In some embodiments of formula (VI) or (VI-a), R⁶ is methyl.

In some embodiments, the compound is represented by the structure of formula (VII):

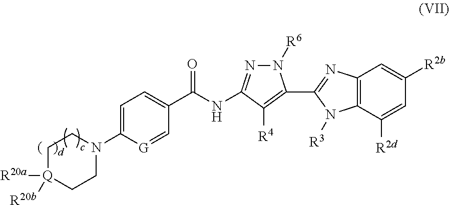

wherein
G is N or CH;
Q is N or C;
each of R^{2b} and R^{2d} is independently alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, 'SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, alkylaryl, alkylaryl, heterocyclyl, or heteroaryl, each of which is independently unsubstituted or substituted, or hydrogen;
each R⁸ is independently alkyl, alkenyl, alkynyl, cycloalkyl, aryl, alkylaryl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, alkylaryl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, alkylaryl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each R^{20a} and R^{20b} is independently:
i) —(CH₂)ₜOR¹⁹, wherein R¹⁹ is hydrogen, a C₁-C₆ alkyl, wherein the C₁-C₆ alkyl is unsubstituted or substituted, or a N, O, or S-containing heterocyclyl;
ii) —C(O)R^{19a}, wherein R^{19a} is C₁-C₆ alkyl or —(CH₂)ₓOR²¹, wherein R²¹ is C₁-C₆ alkyl that is unsubstituted or substituted with NH₂ or OH, wherein x is 0 or 1;
iii) —NR²²R²³, wherein R²² and R²³ are independently C₁-C₆ alkyl, or R²² and R²³, together with the nitrogen to which are R²² and R²³ are attached, form an unsubstituted or substituted heterocyclic ring;
iv) —OR²⁴, wherein R²⁴ is C₁-C₆ alkyl;
v) an oxygen-containing heterocyclic ring;
vi) —C₁-C₆ alkyl, —OH, or absent; or
vii) hydrogen, provided that at least one of R^{20a} and R^{20b} is not hydrogen; or R^{20a} and R^{20b} together with Q form an oxygen-containing heterocyclic ring;
c is 0 or 1;
d is 0 or 1;
each q is independently 1, 2, 3, 4, 5, or 6; and
t is 0, 1, 2, 3, 4, 5 or 6;
   or a pharmaceutically-acceptable salt or a tautomer thereof.

In some embodiments the disclosure provides compounds of formula (VII-a):

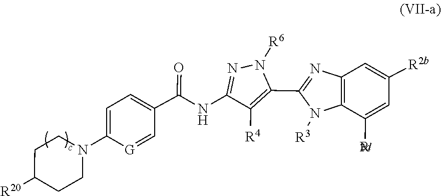

wherein
G is N or CH;
Q is N or CH;
each of R^{2b} and R^{2d} is independently alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each of R¹⁷ and R¹⁸ is independently hydrogen or alkyl; and
R²⁰ is —(CH₂)ₜOR¹⁹, wherein R¹⁹ is hydrogen, a C₁-C₆ alkyl, or a N-, O-, or S-containing heterocyclyl;
c is 0 or 1;
each q is independently 1, 2, 3, 4, 5, or 6; and
t is 0, 1, 2, 3, 4, 5 or 6;
   or a pharmaceutically-acceptable salt or a tautomer thereof.

In some embodiments of formula (VII) or (VH-a), R^{2b} is H. In some embodiments of formula (VII), R^{2b} is OR⁷.

In some embodiments of formula (VII) or (VH-a), R^{2d} is H. In some embodiments of formula (VII), R^{2d} is halogen, e.g., fluorine.

In some embodiments of formula (VII) or (VH-a), R³ is hydrogen.

In some embodiments of formula (VII) or (VH-a), R⁴ is hydrogen.

In some embodiments of formula (VII) or (VII-a), R⁶ is methyl.

In some embodiments, the present disclosure provides a compound that is N-[5-(1H-benzimidazol-2-yl)-1-methyl-pyrazol-3-yl]-4-morpholino-benzamide, or a pharmaceutically-acceptable salt or a tautomer thereof. In some embodiments, the present disclosure provides a compound that is N-[5-(1H-benzimidazol-2-yl)-1-methyl-pyrazol-3-yl]-6-[4-(hydroxymethyl)-1-piperidyl]pyridine-3-carboxamide, or a pharmaceutically-acceptable salt or a tautomer thereof. In some embodiments, the present disclosure provides a compound that is N-[5-(1H-benzimidazol-2-yl)-1H-pyrazol-3-yl]-4-methylsulfonyl-benzamide, or a pharmaceutically-acceptable salt or a tautomer thereof. In some embodiments, the present disclosure provides a compound that is N-[5-(1H-benzimidazol-2-yl)-1-methyl-pyrazol-3-yl]-6-(3-methoxyazetidin-1-yl)pyridine-3-carboxamide, or a pharmaceutically-acceptable salt or a tautomer thereof. In some embodiments, the present disclosure provides a compound that is N-[5-(1H-benzimidazol-2-yl)-1-[(4-methoxyphenyl) methyl]pyrazol-3-yl]-3-chloro-4-methoxy-benzamide, or a pharmaceutically-acceptable salt or a tautomer thereof.

In some embodiments, the present disclosure provides a compound that is N-[5-(1H-benzimidazol-2-yl)-1H-pyrazol-3-yl]-6-(1-oxa-8-azaspiro[4.5]decan-8-yl)pyridine-3-carboxamide, or a pharmaceutically-acceptable salt or a tautomer thereof. In some embodiments, the present disclosure provides a compound that is 6-[4-(dimethylamino)-1-piperidyl]-N-[5-(5-fluoro-1H-benzimidazol-2-yl)-1-methyl-pyrazol-3-yl]pyridine-3-carboxamide, or a pharmaceutically-acceptable salt or a tautomer thereof. In some embodiments, the present disclosure provides a compound that is N-[5-(1H-benzimidazol-2-yl)-1H-pyrazol-3-yl]-6-[4-(hydroxymethyl)-1-piperidyl]pyridine-3-carboxamide, or a pharmaceutically-acceptable salt or a tautomer thereof. In some embodiments, the present disclosure provides a compound that is N-[5-(1H-benzimidazol-2-yl)-1H-pyrazol-3-yl]-4-[4-(2-methoxyethyl)piperazin-1-yl]benzamide, or a pharmaceutically-acceptable salt or a tautomer thereof. In some embodiments, the present disclosure provides a compound that is N-[5-(1H-benzimidazol-2-yl)-1-methyl-pyrazol-3-yl]-6-[4-(oxetan-3-yl)piperazin-1-yl]pyridine-3-carboxamide, or a pharmaceutically-acceptable salt or a tautomer thereof. In some embodiments, the present disclosure provides a compound that is N-[5-(5-chloro-1H-benzimidazol-2-yl)-1H-pyrazol-3-yl]-6-(4-hydroxy-1-piperidyl)pyridine-3-carboxamide, or a pharmaceutically-acceptable salt or a tautomer thereof.

In some embodiments, the present disclosure provides a compound that is N-[5-(1H-benzimidazol-2-yl)-1-methyl-pyrazol-3-yl]-6-(4-methylpiperazin-1-yl)pyridine-3-carboxamide, or a pharmaceutically-acceptable salt or a tautomer thereof. In some embodiments, the present disclosure provides a compound that is -[5-(1H-benzimidazol-2-yl)-1H-pyrazol-3-yl]-4-[(3R)-3-hydroxypyrrolidin-1-yl]benzamide, or a pharmaceutically-acceptable salt or a tautomer thereof. In some embodiments, the present disclosure provides a compound that is N-[5-(1H-benzimidazol-2-yl)-1-methyl-pyrazol-3-yl]-4-[4-(oxetan-3-yl)piperazin-1-yl]benzamide, or a pharmaceutically-acceptable salt or a tautomer thereof. In some embodiments, the present disclosure provides a compound that is N-[5-(1H-benzimidazol-2-yl)-1H-pyrazol-3-yl]-4-[4-(oxetan-3-yl)piperazin-1-yl]benzamide, or a pharmaceutically-acceptable salt or a tautomer thereof. In some embodiments, the present disclosure provides a compound that is N-[5-(1H-benzimidazol-2-yl)-1H-pyrazol-3-yl]-3-cyano-4-(2-hydroxyethoxy)benzamide, or a pharmaceutically-acceptable salt or a tautomer thereof.

In some embodiments, the present disclosure provides a compound that is N-[5-(5-fluoro-1H-benzimidazol-2-yl)-1-methyl-pyrazol-3-yl]-6-(4-methoxy-1-piperidyl)pyridine-3-carboxamide, or a pharmaceutically-acceptable salt or a tautomer thereof. In some embodiments, the present disclosure provides a compound that is N-[5-(1H-benzimidazol-2-yl)-1-methyl-pyrazol-3-yl]-4-(2-hydroxyethoxy)-3-(trifluoromethyl)benzamide, or a pharmaceutically-acceptable salt or a tautomer thereof. In some embodiments, the present disclosure provides a compound that is N-[5-(1H-benzimidazol-2-yl)-1H-pyrazol-3-yl]-2-(4-methylpiperazin-1-yl)pyrimidine-5-carboxamide, or a pharmaceutically-acceptable salt or a tautomer thereof. In some embodiments, the present disclosure provides a compound that is N-[5-(1H-benzimidazol-2-yl)-1-methyl-pyrazol-3-yl]-6-[(3R,5S)-3,5-dimethylpiperazin-1-yl]pyridine-3-carboxamide, or a pharmaceutically-acceptable salt or a tautomer thereof. In some embodiments, the present disclosure provides a compound that is 6-(4-acetylpiperazin-1-yl)-N-[5-(1H-benzimidazol-2-yl)-1-methyl-pyrazol-3-yl]pyridine-3-carboxamide, or a pharmaceutically-acceptable salt or a tautomer thereof.

In some embodiments, the present disclosure provides pharmaceutical composition comprising any compound of the disclosure, or a pharmaceutically-acceptable salt or tautomer thereof, and a pharmaceutically-acceptable excipient in a unit dosage form.

In some embodiments, the present disclosure provides a method a treating a condition, the method comprising administering to a subject in need thereof a therapeutically-effective amount of a compound of the disclosure. In some embodiments, the present disclosure provides pharmaceutical composition comprising any compound of formula (I), or a pharmaceutically-acceptable salt or tautomer thereof, and a pharmaceutically-acceptable excipient in a unit dosage form, for use in a method of treating a condition in a subject in need thereof. In some embodiments, the condition is diabetes. In some embodiments, the condition is insulin-dependent diabetes. In some embodiments, the insulin-dependent diabetes is type 1 diabetes mellitus (T1DM). In some embodiments, the insulin-dependent diabetes is diabetes secondary to pancreatectomy. In some embodiments, the condition is non-insulin-dependent diabetes. In some embodiments, the condition is type 2 diabetes or gestational diabetes.

In some embodiments, the present disclosure provides a method of treating a condition, the method comprising administering to a subject in need thereof a therapeutically-effective amount of a compound of the disclosure, or a pharmaceutically-acceptable salt or tautomer thereof. In some embodiments, the condition is diabetes. In some embodiments, the condition is diabetes. In some embodiments, the condition is insulin-dependent diabetes. In some embodiments, the insulin-dependent diabetes is type 1 diabetes mellitus (T1DM). In some embodiments, the insulin-dependent diabetes is diabetes secondary to pancreatectomy. In some embodiments, the condition is non-insulin-dependent diabetes. In some embodiments, the condition is type 2 diabetes or gestational diabetes.

In some embodiments, the present disclosure provides a method of treating a condition, the method comprising administering to a subject in need thereof a therapeutically-effective amount of a pharmaceutical composition comprising a compound of the disclosure, or a pharmaceutically-acceptable salt or tautomer thereof, and a pharmaceutically-acceptable excipient in a unit dosage form.

In some embodiments, the present disclosure provides a compound of the disclosure, or a pharmaceutically-acceptable salt or tautomer thereof, for use in a method of treating a condition in a subject in need thereof.

Several moieties in a compound of the disclosure are optionally substituted. Non-limiting examples of optional substituents include a hydroxyl group, sulfhydryl group, halogen, amino group, nitro group, nitroso group, cyano group, azido group, sulfoxide group, sulfone group, sulfonamide group, carboxyl group, carboxaldehyde group, imine group, alkyl group, halo-alkyl group, alkenyl group, halo-alkenyl group, alkynyl group, halo-alkynyl group, alkoxy group, aryl group, aryloxy group, aralkyl group, arylalkoxy group, heterocyclyl group, acyl group, acyloxy group, carbamate group, amide group, ureido group, epoxy group, and ester group.

Non-limiting examples of alkyl and alkylene groups include straight, branched, and cyclic alkyl (cycloalkyl) and alkylene groups. An alkyl or alkylene group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of substituted alkyl groups includes hydroxymethyl, chloromethyl, fluoromethyl, difluoromethyl, trifluoromethyl, aminomethyl, 1-chloroethyl, 2-hydroxyethyl, 1,2-difluoroethyl, and 3-carboxypropyl.

Non-limiting examples of cyclic alkyl groups (cycloalkyl) include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-1-en-1-yl, isopropenyl, but-1-en-4-yl; 2-chloroethenyl, 4-hydroxybuten-1-yl, 7-hydroxy-7-methyloct-4-en-2-yl, and 7-hydroxy-7-methyloct-3,5-dien-2-yl.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C_{9,}

C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl or alkynylene groups include ethynyl, prop-2-yn-1-yl, prop-1-yn-1-yl, and 2-methyl-hex-4-yn-1-yl; 5-hydroxy-5-methylhex-3-yn-1-yl, 6-hydroxy-6-methylhept-3-yn-2-yl, and 5-hydroxy-5-ethylhept-3-yn-1-yl.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy. Alkoxy groups can be, for example, substituted or unsubstituted. Alkoxy group can be substituted for example, with oxo, halogen, amino, nitrile, nitro, hydroxyl, haloalkyl, alkoxy, aryl, cycloalkyl, heterocycloalkyl, heteroaryl.

A haloalkoxy group is an alkoxy group that is substituted by one or more halogen atoms, i.e., F, Cl, Br or I. Non-limiting examples of haloalkoxy groups include trifluoromethoxy, trichloromethoxy, tribromomethoxy, triiodomethoxy, trifluoroethoxy, trichloroethoxy, tribromoethoxy, triiodoethoxy, trifluoropropoxy, trichlorompropoxy, tribromopropoxy, triiodopropoxy, trifluoroisopropoxy, trichloromisopropoxy, tribromoisopropoxy, triiodoisopropoxy trifluoroisobutoxy, trichloromisobutoxy, tribromoixobutoxy, and triiodoisobutoxy. A haloalkoxy group can be substituted, for example, with amino, nitrile, nitro, hydroxyl, haloalkyl, alkoxy, aryl, cycloalkyl, heterocycloalkyl, heteroaryl, and the like. For example, a halogen or hydrogen group of a haloalkoxy group can be optionally replaced by amino, nitrile, nitro, hydroxyl, haloalkyl, alkoxy, aryl, cycloalkyl, heterocycloalkyl, and heteroaryl.

An amine is a group NH₂. An alkylamine is an amine substituted by one or more alkyl groups. An arylamine is an amine that is substituted by one or more alkyl groups. An heterocyclylamine is an amine substituted by one or more heterocyclic groups. A heteroarylamine is an amine substituted by one or more heteroaryl groups.

An aryl group can be heterocyclic or non-heterocyclic. An aryl group can be monocyclic or polycyclic. An aryl group can be substituted with any number of substituents described herein, for example, halogen, amino, nitrile, nitro, hydroxyl, alkyl, alkenyl, alkynyl, haloalkyl, alkoxy, aryl, cycloalkyl, heterocycloalkyl, and heteroaryl. An aryl group can be substituted with one substituent, or two or more substituents that are the same or different. Non-limiting examples of aryl groups include phenyl, toluyl, naphthyl, pyrrolyl, pyridyl, imidazolyl, thiophenyl, and furyl. Non-limiting examples of substituted aryl groups include 3,4-dimethylphenyl, 4-tert-butylphenyl, 4-cyclopropylphenyl, 4-diethylaminophenyl, 4-(trifluoromethyl)phenyl, 4-(difluoromethoxy)-phenyl, 4-(trifluoromethoxy)phenyl, 3-chlorophenyl, 4-chlorophenyl, 3,4-dichlorophenyl, 2-fluorophenyl, 2-chlorophenyl, 2-iodophenyl, 3-iodophenyl, 4-iodophenyl, 2-methylphenyl, 3-fluorophenyl, 3-methylphenyl, 3-methoxyphenyl, 4-fluorophenyl, 4-methylphenyl, 4-methoxyphenyl, 2,3-difluorophenyl, 3,4-difluorophenyl, 3,5-difluorophenyl, 2,3-dichlorophenyl, 3,4-dichlorophenyl, 3,5-dichlorophenyl, 2-hydroxyphenyl, 3-hydroxyphenyl, 4-hydroxyphenyl, 2-methoxyphenyl, 4-methoxyphenyl, 2,3-dimethoxyphenyl, 3,4-dimethoxyphenyl, 3,5-dimethoxyphenyl, 2,4-difluorophenyl, 2,5-difluorophenyl, 2,6-difluorophenyl, 2,3,4-trifluorophenyl, 2,3,5-trifluorophenyl, 2,3,6-trifluorophenyl, 2,4,5-trifluorophenyl, 2,4,6-trifluorophenyl, 2,4-dichlorophenyl, 2,5-dichlorophenyl, 2,6-dichlorophenyl, 3,4-dichlorophenyl, 2,3,4-trichlorophenyl, 2,3,5-trichlorophenyl, 2,3,6-trichlorophenyl, 2,4,5-trichlorophenyl, 3,4,5-trichlorophenyl, 2,4,6-trichlorophenyl, 2,3-dimethylphenyl, 2,4-dimethylphenyl, 2,5-dimethylphenyl, 2,6-dimethylphenyl, 2,3,4-trimethylphenyl, 2,3,5-trimethylphenyl, 2,3,6-trimethylphenyl, 2,4,5-trimethylphenyl, 2,4,6-trimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 2,3,4-triethylphenyl, 2,3,5-triethylphenyl, 2,3,6-triethylphenyl, 2,4,5-triethylphenyl, 2,4,6-triethylphenyl, 2-isopropylphenyl, 3-isopropylphenyl, and 4-isopropylphenyl.

Non-limiting examples of substituted aryl groups include 2-aminophenyl, 2-(N-methylamino)phenyl, 2-(N,N-dimethylamino)phenyl, 2-(N-ethylamino)phenyl, 2-(N,N-diethylamino)phenyl, 3-aminophenyl, 3-(N-methylamino)phenyl, 3-(N,N-dimethylamino)phenyl, 3-(N-ethylamino)phenyl, 3-(N,N-diethylamino)phenyl, 4-aminophenyl, 4-(N-methylamino)phenyl, 4-(N,N-dimethylamino)phenyl, 4-(N-ethylamino)phenyl, and 4-(N,N-diethylamino)phenyl.

An aryloxy group can be, for example, an oxygen atom substituted with any aryl group. An ether or an ether group comprises an aryloxy group. The aryloxy group can be substituted or unsubstituted.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

Non-limiting examples of heterocycles include: heterocyclic units having a single ring containing one or more heteroatoms, non-limiting examples of which include, diazirinyl, aziridinyl, azetidinyl, oxetanyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1H-azepinyl, 2,3-dihydro-1H-indole, and 1,2,3,4-tetrahydroquinoline; and ii) heterocyclic units having 2 or more rings one of which is a heterocyclic ring, non-limiting examples of which include hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl.

Non-limiting examples of heteroaryl include: i) heteroaryl rings containing a single ring, non-limiting examples of which include, 1,2,3,4-tetrazolyl, [1,2,3]triazolyl, [1,2,4]triazolyl, triazinyl, thiazolyl, 1H-imidazolyl, pyrazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl.

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutically-Acceptable Salts.

Any compound herein can be provided as a pharmaceutically-acceptable salt. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt.

Metal salts can arise from the addition of an inorganic base to a compound of the present disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, imidazole, or pyrazine.

In some embodiments, an ammonium salt is a triethyl amine salt, a trimethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, a pyridazine salt, a pyrimidine salt, an imidazole salt, or a pyrazine salt.

Acid addition salts can arise from the addition of an acid to a compound of the present disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, or a maleate salt.

### Pharmaceutical Compositions of the Disclosure.

In some embodiments, the present disclosure provides a pharmaceutical composition comprising a compound of the disclosure, and a pharmaceutically-acceptable excipient in a unit dosage form. A pharmaceutical composition of the disclosure can be used, for example, before, during, or after treatment of a subject with, for example, another pharmaceutical agent.

Subjects can be, for example, elderly adults, adults, adolescents, pre-adolescents, children, toddlers, infants, neonates, and non-human animals. In some embodiments, a subject is a patient.

A pharmaceutical composition of the disclosure can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, oral, intrarectal, parenteral, ophthalmic, subcutaneous, transdermal, nasal, vaginal, and topical administration.

A pharmaceutical composition can be administered in a local manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated by combining the active compounds with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

The active compounds can be modified to target selected cells and then administered systemically, e.g., by oral ingestion or by intrarectal administration. For example, the active compounds can be modified such that they specifically bind to receptors or antigens expressed on a selected cell surface, e.g., by linking the active compounds to peptides, antibodies, aptamers, or small molecules that bind to cell surface receptors or antigens expressed by enteroendocrine cells in the gut.

Pharmaceutical preparations can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

The compounds of the disclosure can be applied topically to the skin, or a body cavity, for example, oral, vaginal, bladder, cranial, spinal, thoracic, or pelvic cavity of a subject. The compounds of the disclosure can be applied to an accessible body cavity. The compounds of the present disclosure can be administered by a variety of means including orally, parenterally, by inhalation spray, topically, or rectally in formulations containing pharmaceutically acceptable carriers, adjuvants and vehicles. The term parenteral as used here includes but is not limited to subcutaneous, intravenous, intramuscular, intraarterial, intradermal, intrathecal and epidural injections with a variety of infusion techniques. Intraarterial and intravenous injection as used herein includes administration through catheters. Administration via intracoronary stents and intracoronary reservoirs is also contemplated. The term oral as used herein includes, but is not limited to sublingual and buccal. Oral administration includes fluid drinks, energy bars, as well as pill formulations.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, and PEG. In suppository forms of the compositions, a low-melting wax such as a mixture of fatty acid glycerides, optionally in combination with cocoa butter, can be melted.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulations can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the disclosure include liquid, powder, gel, nanosuspension, nanoparticle, microgel, aqueous or oily suspensions, emulsion, and any combination thereof.

Non-limiting examples of pharmaceutically-acceptable excipients suitable for use in the disclosure include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

Pharmaceutical compositions of the disclosure can be contained in enteric forms to survive the stomach or further coated or mixed to be released in a particular region of the gastrointestinal tract by known methods. For the purpose of oral therapeutic administration, the active agent can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL® or corn starch; a lubricant such as magnesium stearate or STEROTES® a glidant such as colloidal silicon dioxide; a sweetening agent such as sucrose or saccharin; or a flavoring agent such as peppermint, methyl salicylate, or orange flavoring.

Examples of enteric release materials include pH-sensitive polymers which provide an aqueous barrier and do not dissolve or disintegrate in acidic aqueous environs typical of the stomach, but which do dissolve or disintegrate in the higher pH aqueous environs typical of the intestines. The time duration of the disintegration upon reaching a higher pH condition dictates where in the intestine the agent is released.

In some embodiments, the disclosure provides an enteric coated tablet comprising a tablet core containing a compound of the disclosure surrounded by an enteric coating. Tablet cores area typically made by mixing granular or powdered active agent with a pharmaceutical carrier and compressing the resulting mixture into a tablet core by conventional means. The tablet core is then coated with an enteric release material by conventional means, such as in a pan coater or a fluidized bed coater. Examples of commercially available enteric release materials which may be used to produce dosage unit forms of the present invention include cellulose acetate trimellitate, hydroxypropyl methylcellulose phthalate, carboxymethylethylcellulose, co-polymerized methacrylic acid/methacrylic acid methyl esters such as, for instance, materials known under the trade name EUDRAGIT® L12.5, L100, or EUDRAGIT® S12.5, S100 or similar compounds used to obtain enteric coatings, methacrylic acid copolymers (Eudragit® L, S and L30D from Rohm Pharma GmbH, Darmstadt, West Germany); cellulose acetate phthalate (Aquateric® from FMC Corp., Philadelphia, Pa.); polyvinyl acetate phthalate (Coterie® from Colorcon Inc., West Point, Pa.); and hydroxypropyl methylcellulose phthalate (HP50 and HP55 from Shin-Etsu Chemical Co., Ltd., Tokyo, Japan). The preferred thickness of enteric coating used is sufficient to protect the active agent from exposure in the stomach but disintegrates rapidly in the intestines, such as in the small intestine, or in the duodenum or jejunum, to expose the active agent, such that it contacts gut cells, such as enteroendocrine cells or enteroendocrine progenitor cells in the gastrointestinal tract.

In some embodiments, the disclosure provides an enteric coated hard gelatin capsule containing a compound of the disclosure. The compound is typically mixed with a pharmaceutical carrier and filled into hard gelatin capsule shells. The capsules are then enteric coated using a coating as described for enteric coated tablets above.

In some embodiments, the disclosure provides enteric coated granules comprising a compound of the disclosure. Granules comprising active agent and a pharmaceutical carrier are prepared and enterically coated using an enteric coating material as described herein above. A dosage unit form of the enteric coated granules can be prepared by blending granules with an appropriate pharmaceutical carrier, and compressing granules into tablets or filling them into hard gelatin capsule shells by conventional means. In some embodiments, the present disclosure provides a soft gelatin capsule containing a solution, suspension or emulsion of active agent. The soft gelatin capsule shell is made of an enteric release material which remains intact in the stomach and prevents exposure of the active agent in the stomach, but which dissolves or disintegrates in the intestines and releases the active agent in the intestine as described above.

Systemic administration can also be by transmucosal means to the intestinal or colon. For transmucosal or transdermal administration, penetrants appropriate to the barrier to be permeated are used in the formulation. Such include, for example, for transmucosal administration, detergents, bile salts, and fusidic acid derivatives. Transmucosal administration can be accomplished through the use of nasal sprays or suppositories. For transdermal administration, the active agents are formulated into ointments, salves, gels, or creams.

A composition of the disclosure can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), each of which is incorporated by reference in its entirety.

In some embodiments, a compound of the disclosure can be delivered by long-term, automated drug delivery to the gut using an osmotic pump to infuse a desired dose of the compound for a desired time. Insulin pumps can be adapted to deliver the compound to the gut. The delivery rate of the compound to control glucose intolerance, diabetes types 1 or 2 can be readily adjusted through a large range to accommodate changing insulin requirements of an individual (e.g., basal rates and bolus doses). New pumps permit a periodic dosing manner, i.e., liquid is delivered in periodic discrete doses of a small fixed volume rather than in a continuous flow manner. The overall liquid delivery rate for the device is controlled and adjusted by controlling and adjusting the dosing period. The pump can be coupled with a continuous blood glucose monitoring device and remote unit. In such an arrangement, the hand-held remote unit that controls the continuous blood glucose monitoring device can wirelessly communicate with and control both the blood glucose monitoring unit and the fluid delivery device delivering therapeutic agents of the present invention. The effective dosage of an active agent used for treatment can increase or decrease over the course of a particular treatment. Changes in dosage may result and become apparent from monitoring the level of insulin and/or monitoring glycemia control in a biological sample, such as blood or serum.

Multiple therapeutic agents can be administered in any order or simultaneously. In some embodiments, a compound of the disclosure is administered in combination with, before, or after treatment with another therapeutic agent. In some embodiments, a compound of the disclosure is administered at regular intervals, such as, for example, twice daily, daily, or weekly, and the second therapeutic agent is administered intermittently or on an as-needed basis. If simultaneously, the multiple therapeutic agents can be provided in a single, unified form, or in multiple forms, for example, as multiple separate pills. The agents can be packed together or separately, in a single package or in a plurality of packages. One or all of the therapeutic agents can be given in multiple doses. If not simultaneous, the timing between the multiple doses can vary to as much as about a month.

Each therapeutic agent can be administered in a separate unit dosage form, or combinations of therapeutic agents can be administered in the same unit dosage form. Routes of administration of each therapeutic agent can be the same, or can differ. For example, a compound of the disclosure can be administered in oral form (e.g. tablet, capsule, pill, pellet, granules, powder, syrup, suspension, emulsion, dispersion, solution), and a second therapeutic agent can be administered by parenteral injection (e.g. intravenous, subcutaneous, intra-arterial, intra-peritonial, or intramuscular injection). For example, a compound of the disclosure can be administered in oral form, and a second therapeutic agent can be administered by oral form. For example, a compound of the disclosure can be administered in oral form, and a second therapeutic agent can be administered by parenteral injection. For example, a compound of the disclosure can be administered by parenteral injection, and a second therapeutic agent can be administered by parenteral injection. For example, a compound of the disclosure can be administered in the same pharmaceutical composition with a second therapeutic agent by parenteral injection. For example, a compound of the disclosure can be administered in the same pharmaceutical composition with a second therapeutic agent in oral form. Additional routes of administration include, but are not limited to, topical administration, administration by inhalation or administration via suppository.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. In some embodiments, the compositions are administered to a subject during or as soon as possible after the onset of the symptoms. The administration of the therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, or about 10 years. For treatment of chronic conditions, a compound of the disclosure can be administered as long as symptoms of the disease are present in a subject. The length of treatment can vary for each subject.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

Depending on the intended mode of administration, the pharmaceutical compositions can be in the form of solid, semi-solid or liquid dosage forms, such as, for example, tablets, suppositories, pills, capsules, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Non-limiting examples of either approved or experimental pharmaceutically active agents suitable for combination with compositions of the disclosure include anti-infectives (e.g., aminoglycosides, antiviral agents, antimicrobials), anticholinergics/antispasmotics, antidiabetic agents, antihypertensive agents, anti-obesity agents, anti-fibrotic agents, antineoplastics, cardiovascular agents, lipid-lowering agents, central nervous system agents, coagulation modifiers, hormones, immunologic agents, immunosuppressive agents, and ophthalmic preparations.

Compounds can be delivered via liposomal technology. The use of liposomes as drug carriers can increase the therapeutic index of the compounds. Liposomes are composed of natural phospholipids and can contain mixed lipid chains with surfactant properties (e.g., egg phosphatidylethanolamine). A liposome design can employ surface ligands for attaching to unhealthy tissue. Non-limiting examples of liposomes include the multilamellar vesicle (MLV), the small unilamellar vesicle (SUV), and the large unilamellar vesicle (LUV). Liposomal physicochemical properties can be modulated to optimize penetration through biological barriers and retention at the site of administration, and to reduce a likelihood of developing premature degradation and toxicity to non-target tissues. Optimal liposomal properties depend on the administration route: large-sized liposomes show good retention upon local injection, small-sized liposomes are better suited to achieve passive targeting. PEGylation reduces the uptake of the liposomes by the liver and spleen, and increases the circulation time, resulting in increased localization at the inflamed site due to the enhanced permeability and retention (EPR) effect. Additionally, liposomal surfaces can be modified to achieve selective delivery of the encapsulated drug to specific target cells. Non-limiting examples of targeting ligands include monoclonal antibodies, vitamins, peptides, and polysaccharides specific for receptors concentrated on the surface of cells associated with the disease.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof.

Compositions of the disclosure can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

In some embodiments, the disclosure provides a method of treating a disease or disorder associated with impaired pancreatic endocrine function, comprising administering a therapeutically effective amount of any compound described herein, or a pharmaceutically acceptable salt or tautomer thereof. In addition to reduced insulin secretion, impaired pancreatic function includes an altered capacity to produce and/or secrete one or more pancreatic hormones including one or more pancreatic peptides such as glucagon, islet amyloid polypeptide (IAPP), pancreatic polypeptide, somatostatin, or ghrelin. Well known pathologies that are associated with impaired pancreatic function include type 1 diabetes and type 2 diabetes. Other pathologies include those sometimes referred to as gestational diabetes, latent autoimmune diabetes of adulthood, maturity onset diabetes of the young, pre-diabetes, impaired fasting glucose, impaired glucose tolerance, fasting hyperglycemia, insulin resistant syndrome, and hyperglycemic conditions. All of these come within the meaning of treating and preventing diabetes.

In some embodiments, the disclosure provides a method of producing cells that make and secrete insulin in a mammal, comprising administering to the mammal an effective amount of any compound described herein or a pharmaceutically acceptable salt or tautomer thereof. In some embodiments, the present disclosure provides a method of treating impaired pancreatic endocrine function, comprising administering a compound that converts enteroendocrine cells into glucose-dependent insulin-producing cells. Compounds described herein can cause enteroendocrine cells such as serotonin (i.e., Tph1) or somatostatin-expressing cells, or Neurogenin3-expressing progenitor in the gut to differentiate into Ins+ cells (e.g. cells that produce insulin). Insulin-producing cells can express markers of mature beta-cells, and secrete insulin and C-peptide in response to glucose and sulfonylureas. Insulin-producing cells can arise primarily from Neurogenin-3 (N3) progenitor cells and also from gut stem cells.

In some embodiments, a compound of the disclosure, or a pharmaceutically-acceptable salt or tautomer thereof, converts enteroendocrine cells into glucose-dependent insulin-producing cells. “Enteroendocrine cells” can refer to insulin-negative endocrine cells in the gastrointestinal tract. Enteroendocrine cells can produce one or more hormones such as gastrin, ghrelin, neuropeptide Y, peptide YY, serotonin, secretin, somatostatin, motilin, cholecystokinin, neurotensin, vasoactive intestinal peptide, glucose-dependent insulinotropic polypeptide or glucagon-like peptide-1. Enteroendocrine cells and any other gut insulin-negative cell capable of differentiating into an insulin-positive cell can be targeted by compounds of the disclosure. For example, insulin-negative gut cells can be contacted with a compound of the disclosure in situ in a subject, or enriched populations of insulin-negative cells can be isolated from the gut, or intestinal explants in culture can be used.

In some embodiments, compounds of the disclosure can be used to treat diseases associated with insulin resistance in a subject. A compound of the disclosure can, for example, stimulate the production of insulin by cells exposed to said compounds. Non-limiting examples of disease associated with insulin resistance that can be treated by a compound of the disclosure include type 1 diabetes mellitus, type 2 diabetes mellitus, metabolic syndrome, hepatitis C, polycystic ovary syndrome, non-alcoholic fatty liver disease/non-alcoholic steatohepatitis, liver cancer, and gestational diabetes.

In some embodiments, a compound of the disclosure exhibits selective activity in favor of FOXO1 over Forkhead box protein A2 (FOXA2), which is another member of the forkhead class of DNA-binding proteins. FOXA2 serves as a transcriptional activator for liver-specific genes such as albumin and transthyretin and also plays important roles in lung and neuronal development.

Efficacy of the methods of treatment described herein can be monitored by determining whether the methods ameliorate any of the symptoms of the disease being treated. Alternatively, one can monitor the level of serum insulin or C-peptide (a byproduct of insulin secretion and an index of functional insulin-secreting cells), the levels of which should increase in response to therapy. Alternatively, efficacy can be measured by monitoring glycemia, glucose tolerance, fat mass, weight gain, ketone bodies or other indicia of the enumerated disease or disorder in the subject being treated.

In some embodiments, the compound exhibits beneficial metabolic stability. Metabolic stability is the susceptibility of a drug to be chemically transformed into other compounds by the organism to which the drug is administered. If metabolic stability is too low, the drug can be rapidly converted to inactive compounds and/or cleared from the organism, thus reducing efficacy. Metabolic stability can be assessed, for example, by measuring how rapidly a drug is metabolized by hepatocytes or liver microsomes.

Compounds described herein can be co-administered with a second therapeutic agent. The second therapeutic agent can be, for example, a drug known to treat pathology associated with pancreatic function, such as sulfonylureas, meglitinides, thiazolidenediones, alpha-glucosidase inhibitors, DPP-4 inhibitors, GLP-1 receptor agonists, SGLT2 inhibitors and insulin. In some embodiments, the second therapeutic agent is metformin, nateglinide, repaglinide, acarbose, pioglitazone, troglitazone, glipizide, glibenclamide, glyburide, glimepiride, or rosiglitazone.

Other examples of a second therapeutic agent include, but are not limited to, angiotensin-converting enzyme (ACE) inhibitors, Rho-associated protein kinase (ROCK) inhibitors, Notch inhibitors, aldosterone antagonists, amphetamines, amphetamine like agents, Angiotensin II receptor antagonists, anti-oxidants, aldose reductase inhibitors, biguanides, gamma secretase inhibitors, sorbitol dehydrogenase inhibitors, thiazolidinediones (glitazones), thiazide and thiazide-like diuretics, triglyceride synthesis inhibitors, uric acid lowering agents, e.g., xanthine oxidase inhibitors, fructokinase inhibitors, and combinations thereof.

ACE inhibitors include, but are not limited to, Benazepril (Lotensin), Captopril, Enalapril (Vasotec), Fosinopril, Lisinopril (Prinivil, Zestril), Moexipril (Univasc), Perindopril (Aceon), Quinapril (Accupril), Ramipril (Altace), Trandolapril (Mavik), and combinations thereof.

Aldosterone antagonists include, but are not limited to, Spironolactone, Eplerenone, Canrenone (canrenoate potassium), Prorenone (prorenoate potassium), Mexrenone (mexrenoate potassium), and combinations thereof.

Amphetamines include, but are not limited to, amphetamine, methamphetamine, methylphenidate, p-methoxyamphetamine, methylenedioxyamphetamine, 2,5-dimethoxy-4-methylamphetamine, 2,4,5-trimethoxyamphetamine, and 3,4-methylenedioxymethamphetamine, N-ethylamphetamine, fenethylline, benzphetamine, and chlorphentermine as well as the amphetamine compounds of Adderall; actedron; actemin; adipan; akedron; allodene; alpha-methyl-(.+−.)-benzeneethanamine; alpha-methylbenzeneethanamine; alpha-methylphenethylamine; amfetamine; amphate; anorexine; benzebar; benzedrine; benzyl methyl carbinamine; benzolone; beta-amino propylbenzene; beta-phenylisopropylamine; biphetamine; desoxynorephedrine; dietamine; DL-amphetamine; elastonon; fenopromin; finam; isoamyne; isomyn; mecodrin; monophos; mydrial; norephedrane; novydrine; obesin; obesine; obetrol; octedrine; oktedrin; phenamine; phenedrine; phenethylamine, alpha-methyl-; percomon; profamina; profetamine; propisamine; racephen; raphetamine; rhinalator, sympamine; simpatedrin; simpatina; sympatedrine; and weckamine. Exemplary amphetamine-like agents include but are not limited to methylphenidate.

Angiotensin II receptor antagonists or angiotensin receptor blockers (ARBs) include, but are not limited to losartan, irbesartan, olmesartan, candesartan, valsartan, and combinations thereof.

Anti-oxidant compounds include but are not limited to L-ascorbic acid or L-ascorbate (vitamin C), menaquinone (vitamin K 2), plastoquinone, phylloquinone (vitamin K 1), retinol (vitamin A), tocopherols (e.g. , α, β, γ and δ-tocotrienols, ubiquinol, and ubiquione (Coenzyme Q10)); and cyclic or polycyclic compounds including acetophenones, anthroquinones, benzoquiones, biflavonoids, catechol melanins, chromones, condensed tannins, coumarins, flavonoids (catechins and epicatechins), hydrolyzable tannins, hydroxycinnamic acids, hydroxybenzyl compounds, isoflavonoids, lignans, naphthoquinones, neolignans, phenolic acids, phenols (including bisphenols and other sterically hindered phenols, aminophenols and thiobisphenols), phenylacetic acids, phenylpropenes, stilbenes and xanthones. Additional cyclic or polycyclic antioxidant compounds include apigenin, auresin, aureusidin, Biochanin A, capsaicin, catechin, coniferyl alcohol, coniferyl aldehyde, cyanidin, daidzein, daphnetin, deiphinidin, emodin, epicatechin, eriodicytol, esculetin, ferulic acid, formononetin, gernistein, gingerol, 3-hydroxybenzoic acid, 4-hydroxybenzoic acid, 3-hydroxycoumarin, juglone, kaemferol, lunularic acid, luteolin, malvidin, mangiferin, 4-methylumbelliferone, mycertin, naringenin, pelargonidin, peonidin, petunidin, phloretin, p-hydroxyacetophenone, (+)-pinoresinol, procyanidin B-2, quercetin, resveratol, resorcinol, rosmaric acid, salicylic acid, scopolein, sinapic acid, sinapoyl-(S)-maleate, sinapyl aldehyde, syrginyl alcohol, telligrandin, umbelliferone, and vanillin. Antioxidants may also be obtained from plant extracts, e.g., from blackberries, blueberries, black carrots, chokecherries, cranberries, black currants, elderberries, red grapes and their juice, hibiscus, oregano, purple sweet potato, red wine, rosemary, strawberries, tea (e.g., black, green or white tea), and from various plant ingredients as ellagic acid.

Aldose reductase inhibitors include, but are not limited to, epalrestat, ranirestat, fidarestat, sorbinil, and combinations thereof. Biguanides include, but are not limited to, metformin, and less rarely used phenformin and buformin, proguanil, and combinations thereof. Thiazolidinediones include, but are not limited to, troglitazone, pioglitazone, ciglitazone, rosiglitazone, englitazone, and combinations thereof. Thiazide and thiazide-like diuretics include, but are not limited to, benzothiadiazine derivatives, chlortalidone, metolazone, and combinations thereof. Exemplary triglyceride synthesis inhibitors include, but are not limited to, diglyceride acyltransferase 1 (DGAT-1) inhibitors. Uric acid lowering agents include, but are not limited to, xanthine oxidase inhibitors, such as allopurinol, oxypurinol, tisopurine, febuxostat, inositols (e.g., phytic acid and myo-inositol), fructokinase inhibitors, and combinations thereof. Fructokinase inhibitors include, but are not limited to, osthol, alpha mangosteen, luteolin, or osthenol.

Suitable second therapeutic agents for use in the present disclosure may also comprise any combinations, prodrugs, pharmaceutically acceptable salts, analogs, and derivatives of the above compounds. Dosing.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A compound described herein can be present in a composition in a range of from about 1 mg to about 2000 mg; from about 100 mg to about 2000 mg; from about 10 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, a dose can be expressed in terms of an amount of the drug divided by the mass of the subject, for example, milligrams of drug per kilograms of subject body mass. In some embodiments, a compound is administered in an amount ranging from about 5 mg/kg to about 50 mg/kg, 250 mg/kg to about 2000 mg/kg, about 10 mg/kg to about 800 mg/kg, about 50 mg/kg to about 400 mg/kg, about 100 mg/kg to about 300 mg/kg, or about 150 mg/kg to about 200 mg/kg.

In some embodiments, described herein is a compound selected from TABLE 1, or a pharmaceutically acceptable salt or tautomer thereof.

TABLE 1

| Structure | Name | Cpd # |
|---|---|---|
| 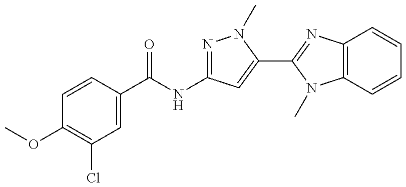 [SMILES: COc1ccc(C(=O)Nc2cc(-c3nc4ccccc4n3C)n(C)n2)cc1Cl] | 3-chloro-4-methoxy-N-[1-methyl-5-(1- methylbenzimidazol-2-yl)pyrazol-3- yl]benzamide | A-1 |
| 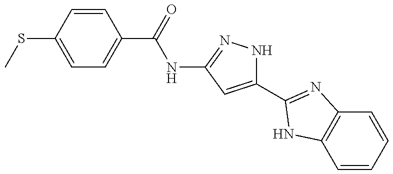 [SMILES: CSc1ccc(C(=O)Nc2cc(-c3nc4ccccc4[nH]3)[nH]n2)cc1] | N-[5-(1H-benzimidazol-2-yl)-1H-pyrazol- 3-yl]-4-methylsulfanyl-benzamide | A-2 |
| 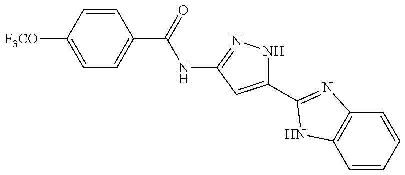 [SMILES: O=C(Nc1cc(-c2nc3ccccc3[nH]2)[nH]n1)c1ccc(OC(F)(F)F)cc1] | N-[5-(1H-benzimidazol-2-yl)-1H-pyrazol- 3-yl]-4-(trifluoro-methoxy)benzamide | A-3 |
| 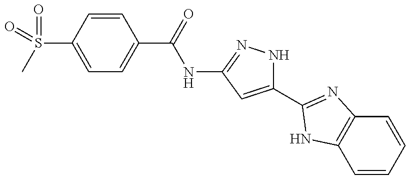 [SMILES: CS(=O)(=O)c1ccc(C(=O)Nc2cc(-c3nc4ccccc4[nH]3)[nH]n2)cc1] | N-[5-(1H-benzimidazol-2-yl)-1H-pyrazol- 3-yl]-4-methylsulfonyl-benzamide | A-4 |
| 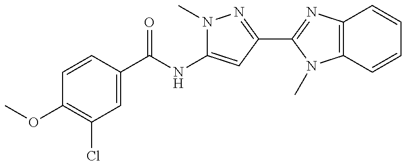 [SMILES: COc1ccc(C(=O)Nc2cc(-c3nc4ccccc4n3C)nn2C)cc1Cl] | 3-chloro-4-methoxy-N-[2-methyl-5-(1- methylbenzimidazol-2-yl)pyrazol-3- yl]benzamide | A-5 |
| 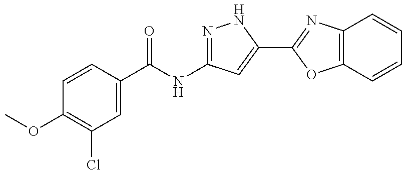 [SMILES: COc1ccc(C(=O)Nc2cc(-c3nc4ccccc4o3)[nH]n2)cc1Cl] | N-[5-(1,3-benzoxazol-2-yl)-1H-pyrazol-3- yl]-3-chloro-4-methoxy-benzamide | A-6 |
| 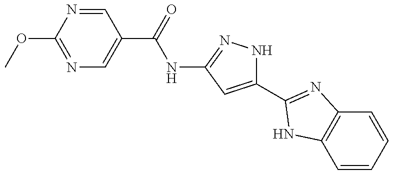 [SMILES: COc1ncc(C(=O)Nc2cc(-c3nc4ccccc4[nH]3)[nH]n2)cn1] | N-[5-(1H-benzimidazol-2-yl)-1H-pyrazol- 3-yl]-2-methoxy-pyrimidine-5- carboxamide | A-7 |
| 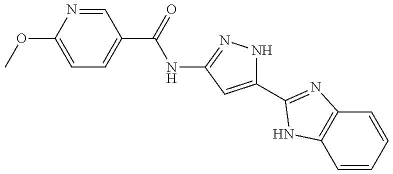 [SMILES: COc1ccc(C(=O)Nc2cc(-c3nc4ccccc4[nH]3)[nH]n2)cn1] | N-[5-(1H-benzimidazol-2-yl)-1H-pyrazol- 3-yl]-6-methoxy-pyridine-3-carboxamide | A-8 |
| 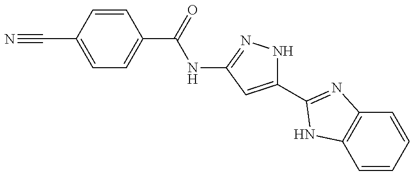 [SMILES: N#Cc1ccc(C(=O)Nc2cc(-c3nc4ccccc4[nH]3)[nH]n2)cc1] | N-[5-(1H-benzimidazol-2-yl)-1H-pyrazol- 3-yl]-4-cyano-benzamide | A-9 |
| 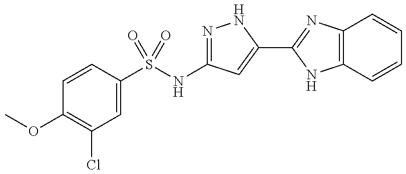 [SMILES: COc1ccc(S(=O)(=O)Nc2cc(-c3nc4ccccc4[nH]3)[nH]n2)cc1Cl] | N-[5-(1H-benzimidazol-2-yl)-1H-pyrazol- 3-yl]-3-chloro-4-methoxy- benzenesulfonamide | A-10 |
| 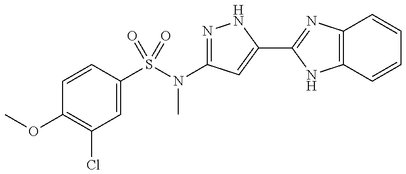 [SMILES: COc1ccc(S(=O)(=O)N(C)c2cc(-c3nc4ccccc4[nH]3)[nH]n2)cc1Cl] | N-[5-(1H-benzimidazol-2-yl)-1H-pyrazol- 3-yl]-3-chloro-4-methoxy-N-methyl- benzenesulfonamide | A-11 |
| 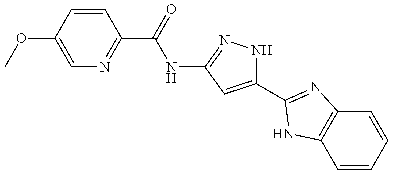 [SMILES: COc1ccc(C(=O)Nc2cc(-c3nc4ccccc4[nH]3)[nH]n2)nc1] | N-[5-(1H-benzimidazol-2-yl)-1H-pyrazol- 3-yl]-5-methoxy-pyridine-2-carboxamide | A-12 |
| 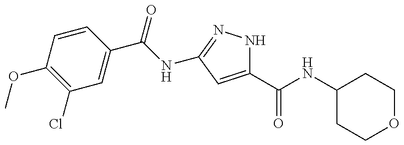 [SMILES: COc1ccc(C(=O)Nc2cc(C(=O)NC3CCOCC3)[nH]n2)cc1Cl] | 3-[(3-chloro-4-methoxy-benzoyl)amino]-N- tetrahydropyran-4-yl-1H-pyrazole-5- carboxamide | A-13 |
| 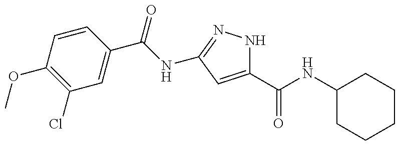 [SMILES: COc1ccc(C(=O)Nc2cc(C(=O)NC3CCCCC3)[nH]n2)cc1Cl] | 3-[(3-chloro-4-methoxy-benzoyl)amino]-N- cyclohexyl-1H-pyrazole-5-carboxamide | A-14 |
| 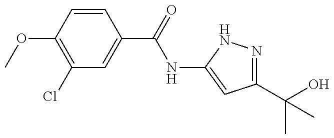 [SMILES: COc1ccc(C(=O)Nc2cc(C(C)(C)O)n[nH]2)cc1Cl] | 3-chloro-N-[3-(1-hydroxy-1-methyl-ethyl)- 1H-pyrazol-5-yl]-4-methoxy-benzamide | A-15 |
| 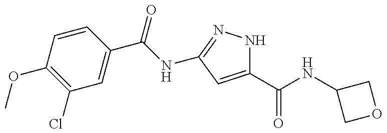 [SMILES: COc1ccc(C(=O)Nc2cc(C(=O)NC3COC3)[nH]n2)cc1Cl] | 3-[(3-chloro-4-methoxy-benzoyl)amino]-N- (oxetan-3-yl)-1H-pyrazole-5-carboxamide | A-16 |
| 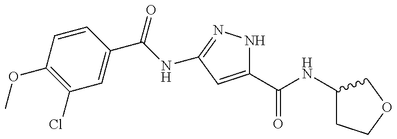 [SMILES: COc1ccc(C(=O)Nc2cc(C(=O)NC3CCOC3)[nH]n2)cc1Cl] | 3-[(3-chloro-4-methoxy-benzoyl)amino]-N- tetrahydrofuran-3-yl-1H-pyrazole-5- carboxamide | A-17 |
| 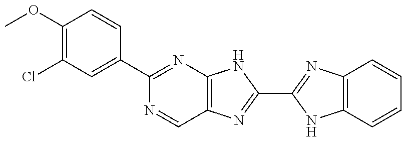 [SMILES: COc1ccc(-c2ncc3nc(-c4nc5ccccc5[nH]4)[nH]c3n2)cc1Cl] | 8-(1H-benzimidazol-2-yl)-2-(3-chloro-4- methoxy-phenyl)-9H-purine | A-18 |
| 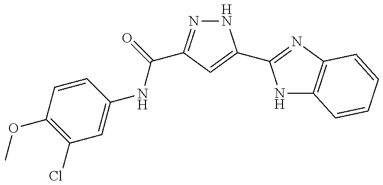 [SMILES: COc1ccc(NC(=O)c2cc(-c3nc4ccccc4[nH]3)[nH]n2)cc1Cl] | 5-(1H-benzimidazol-2-yl)-N-(3-chloro-4- methoxy-phenyl)-1H-pyrazole-3- carboxamide | A-19 |
| 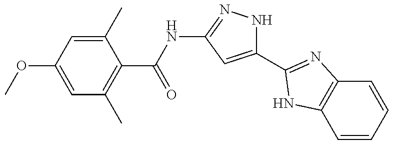 [SMILES: COc1cc(C)c(C(=O)Nc2cc(-c3nc4ccccc4[nH]3)[nH]n2)c(C)c1] | N-[5-(1H-benzimidazol-2-yl)-1H-pyrazol- 3-yl]-4-methoxy-2,6-dimethyl-benzamide | A-20 |
| 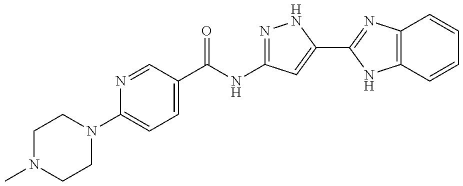 [SMILES: CN1CCN(c2ccc(C(=O)Nc3cc(-c4nc5ccccc5[nH]4)[nH]n3)cn2)CC1] | N-[5-(1H-benzimidazol-2-yl)-1H-pyrazol- 3-yl]-6-(4-methylpiperazin-1-yl)pyridine- 3-carboxamide | A-21 |
| 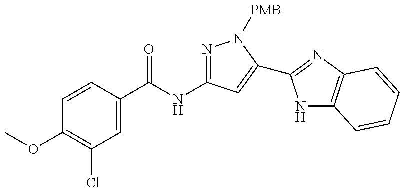 | N-[5-(1H-benzimidazol-2-yl)-1-[(4- methoxyphenyl)methyl]pyrazol-3-yl]-3- chloro-4-methoxy-benzamide | A-22 |
| 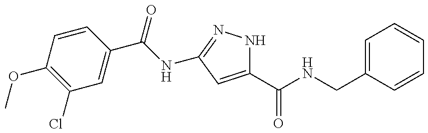 [SMILES: COc1ccc(C(=O)Nc2cc(C(=O)NCc3ccccc3)[nH]n2)cc1Cl] | N-benzyl-3-[(3-chloro-4-methoxy- benzoyl)amino]-1H-pyrazole-5- carboxamide | A-23 |
| 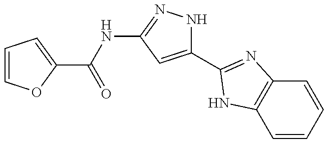 [SMILES: O=C(Nc1cc(-c2nc3ccccc3[nH]2)[nH]n1)c1ccco1] | N-[5-(1H-benzimidazol-2-yl)-1H-pyrazol- 3-yl]furan-2-carboxamide | A-24 |
| 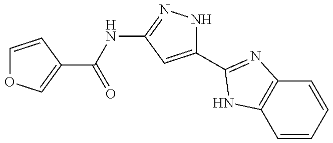 [SMILES: O=C(Nc1cc(-c2nc3ccccc3[nH]2)[nH]n1)c1ccoc1] | N-[5-(1H-benzimidazol-2-yl)-1H-pyrazol- 3-yl]furan-3-carboxamide | A-25 |
| 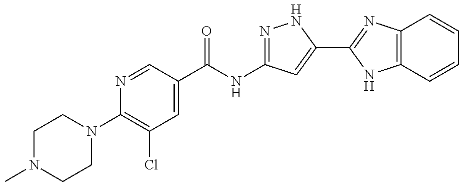 [SMILES: CN1CCN(c2ncc(C(=O)Nc3cc(-c4nc5ccccc5[nH]4)[nH]n3)cc2Cl)CC1] | N-[5-(1H-benzimidazol-2-yl)-1H-pyrazol- 3-yl]-5-chloro-6-(4-methylpiperazin-1-yl) pyridine-3-carboxamide | A-26 |
| 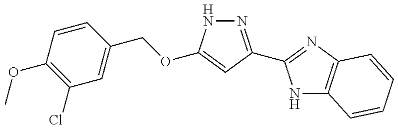 [SMILES: COc1ccc(COc2cc(-c3nc4ccccc4[nH]3)n[nH]2)cc1Cl] | 2-[5-[(3-chloro-4-methoxy- phenyl)methoxy]- 1H-pyrazol-3-yl]-1H-benzimidazole | A-27 |
| 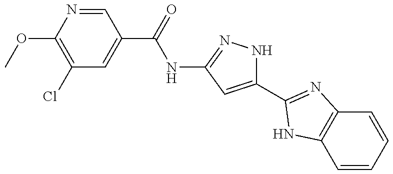 [SMILES: COc1ncc(C(=O)Nc2cc(-c3nc4ccccc4[nH]3)[nH]n2)cc1Cl] | N-[5-(1H-benzimidazol-2-yl)-1H-pyrazol- 3-yl]-5-chloro-6-methoxy-pyridine-3- carboxamide | A-28 |
| 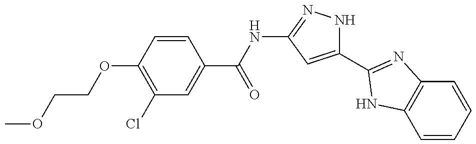 [SMILES: COCCOc1ccc(C(=O)Nc2cc(-c3nc4ccccc4[nH]3)[nH]n2)cc1Cl] | N-[5-(1H-benzimidazol-2-yl)-1H-pyrazol- 3-yl]-3-chloro-4-(2- methoxyethoxy)benzamide | A-29 |
| 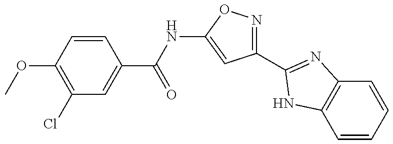 [SMILES: COc1ccc(C(=O)Nc2cc(-c3nc4ccccc4[nH]3)no2)cc1Cl] | N-[3-(1H-benzimidazol-2-yl)isoxazol-5- yl]-3-chloro-4-methoxy-benzamide | A-30 |
| 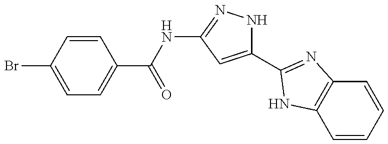 [SMILES: O=C(Nc1cc(-c2nc3ccccc3[nH]2)[nH]n1)c1ccc(Br)cc1] | N-[5-(1H-benzimidazol-2-yl)-1H-pyrazol- 3-yl]-4-bromo-benzamide | A-31 |
| 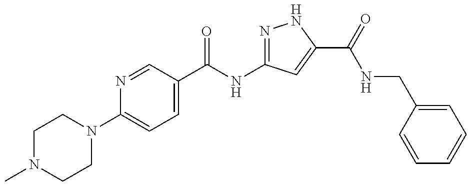 [SMILES: CN1CCN(c2ccc(C(=O)Nc3cc(C(=O)NCc4ccccc4)[nH]n3)cn2)CC1] | N-[5-(benzylcarbamoyl)-1H-pyrazol-3-yl]- 6-(4-methylpiperazin-1-yl)pyridine-3- carboxamide | A-32 |
| 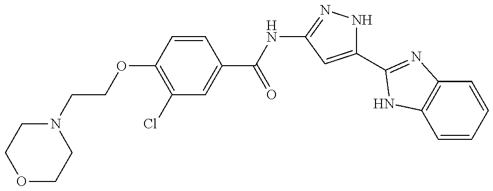 [SMILES: O=C(Nc1cc(-c2nc3ccccc3[nH]2)[nH]n1)c1ccc(OCCN2CCOCC2)c(Cl)c1] | N-[5-(1H-benzimidazol-2-yl)-1H-pyrazol- 3-yl]-3-chloro-4-(2- morpholinoethoxy)benzamide | A-33 |
| 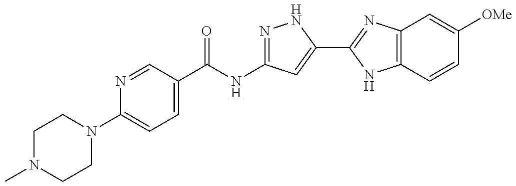 [SMILES: COc1ccc2[nH]c(-c3cc(NC(=O)c4ccc(N5CCN(C)CC5)nc4)n[nH]3)nc2c1] | N-[5-(5-methoxy-1H-benzimidazol-2-yl)- 1H-pyrazol-3-yl]-6-(4-methylpiperazin-1- yl)pyridine-3-carboxamide | A-34 |
| 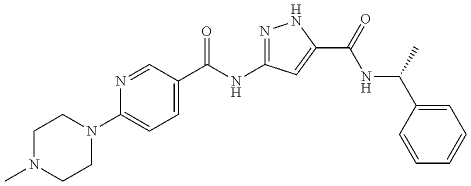 [SMILES: C[C@@H](NC(=O)c1cc(NC(=O)c2ccc(N3CCN(C)CC3)nc2)n[nH]1)c1ccccc1] | 6-(4-methylpiperazin-1-yl)-N-[5-[[(1R)-1- phenylethyl]-carbamoyl]-1H-pyrazol-3- yl]pyridine-3-carboxamide | A-35 |
| 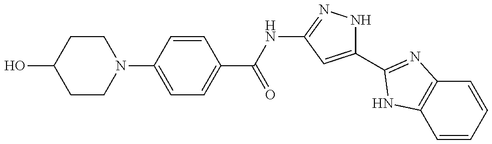 [SMILES: O=C(Nc1cc(-c2nc3ccccc3[nH]2)[nH]n1)c1ccc(N2CCC(O)CC2)cc1] | N-[5-(1H-benzimidazol-2-yl)-1H- pyrazol-3- yl]-4-(4-hydroxy-1-piperidyl)benzamide | A-36 |
| 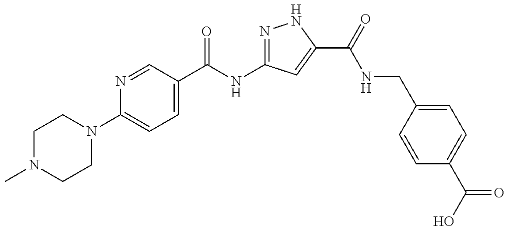 [SMILES: CN1CCN(c2ccc(C(=O)Nc3cc(C(=O)NCc4ccc(C(=O)O)cc4)[nH]n3)cn2)CC1] | 4-[[[3-[[6-(4-methylpiperazin-1- yl)pyridine- 3-carbonyl]amino]-1H-pyrazole-5- carbonyl]amino]methyl]benzoic acid | A-37 |
| 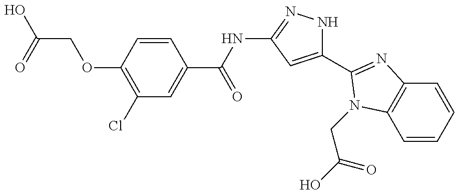 [SMILES: O=C(O)COc1ccc(C(=O)Nc2cc(-c3nc4ccccc4n3CC(=O)O)[nH]n2)cc1Cl] | 2-[2-[3-[[4-(carboxymethoxy)-3-chloro- benzoyl]amino]-1H-pyrazol-5- yl]benzimidazol-1-yl]acetic acid | A-38 |
| 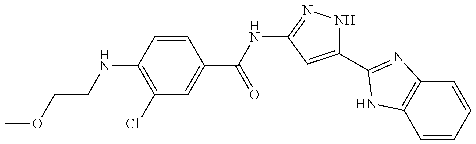 [SMILES: COCCNc1ccc(C(=O)Nc2cc(-c3nc4ccccc4[nH]3)[nH]n2)cc1Cl] | N-[5-(1H-benzimidazol-2-yl)-1H-pyrazol- 3-yl]-3-chloro-4-(2- methoxyethylamino)benzamide | A-39 |
| 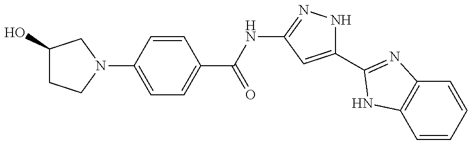 [SMILES: O=C(Nc1cc(-c2nc3ccccc3[nH]2)[nH]n1)c1ccc(N2CC[C@@H](O)C2)cc1] | N-[5-(1H-benzimidazol-2-yl)-1H-pyrazol- 3-yl]-4-[(3R)-3-hydroxypyrrolidine-1- yl]benzamide | A-40 |
| 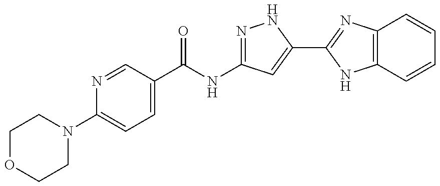 [SMILES: O=C(Nc1cc(-c2nc3ccccc3[nH]2)[nH]n1)c1ccc(N2CCOCC2)nc1] | N-[5-(1H-benzimidazol-2-yl)-1H-pyrazol- 3-yl]-6-morpholino-pyridine-3- carboxamide | A-41 |
| 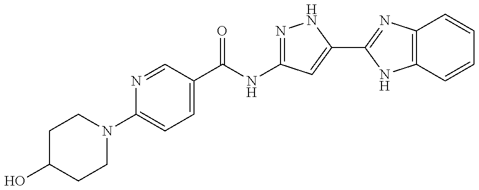 [SMILES: O=C(Nc1cc(-c2nc3ccccc3[nH]2)[nH]n1)c1ccc(N2CCC(O)CC2)nc1] | N-[5-(1H-benzimidazol-2-yl)-1H-pyrazol- 3-yl]-6-(4-hydroxy-1-piperidyl)pyridine-3- carboxamide | A-42 |
| 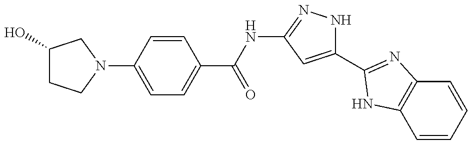 [SMILES: O=C(Nc1cc(-c2nc3ccccc3[nH]2)[nH]n1)c1ccc(N2CC[C@H](O)C2)cc1] | N-[5-(1H-benzimidazol-2-yl)-1H-pyrazol- 3-yl]-4-[(3S)-3-hydroxypyrrolidin-1- yl]benzamide | A-43 |
| 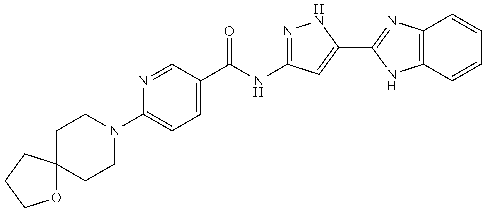 [SMILES: O=C(Nc1cc(-c2nc3ccccc3[nH]2)[nH]n1)c1ccc(N2CCC3(CCCO3)CC2)nc1] | N-[5-(1H-benzimidazol-2-yl)-1H-pyrazol- 3-yl]-6-(1-oxa-8-azaspiro[4.5]decan-8- yl)pyridine-3-carboxamide | A-44 |
| 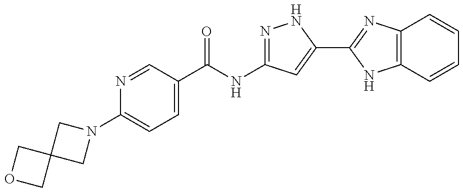 [SMILES: O=C(Nc1cc(-c2nc3ccccc3[nH]2)[nH]n1)c1ccc(N2CC3(COC3)C2)nc1] | N-[5-(1H-benzimidazol-2-yl)-1H-pyrazol- 3-yl]-6-(2-oxa-6-azaspiro[3.3]heptan-6- yl)pyridine-3-carboxamide | A-45 |
| 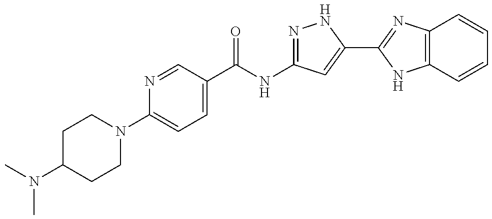 [SMILES: CN(C)C1CCN(c2ccc(C(=O)Nc3cc(-c4nc5ccccc5[nH]4)[nH]n3)cn2)CC1] | N-[5-(1H-benzimidazol-2-yl)-1H-pyrazol- 3-yl]-6-[4-(dimethylamino)-1- piperidyl]pyridine-3-carboxamide | A-46 |
| 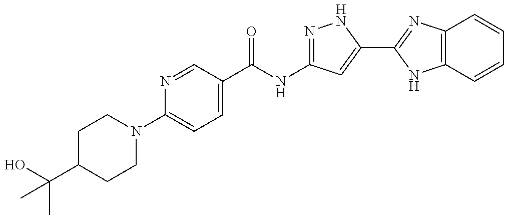 [SMILES: CC(C)(O)C1CCN(c2ccc(C(=O)Nc3cc(-c4nc5ccccc5[nH]4)[nH]n3)cn2)CC1] | N-[5-(1H-benzimidaozl-2-yl)-1H-pyrazol- 3-yl]-6-[4-(1-hydroxy-1-methyl-ethyl)-1- piperidyl]pyridine-3-carboxamide | A-47 |
| 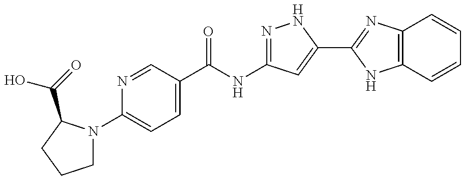 [SMILES: O=C(Nc1cc(-c2nc3ccccc3[nH]2)[nH]n1)c1ccc(N2CCC[C@H]2C(=O)O)nc1] | (2S)-1-[5-[[5-(1H-benzimidazol-2-yl)-1H- pyrazol-3-yl]carbamoyl]-2- pyridyl]pyrrolidine-2-carboxylic acid | A-48 |
| 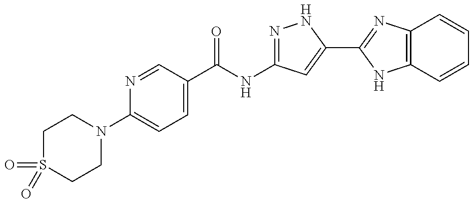 [SMILES: O=C(Nc1cc(-c2nc3ccccc3[nH]2)[nH]n1)c1ccc(N2CCS(=O)(=O)CC2)nc1] | N-[5-(1H-benzimidazol-2-yl)-1H-pyrazol- 3-yl]-6-(1,1-dioxo-1,4-thiazinan-4- yl)pyridine-3-carboxamide | A-49 |
| 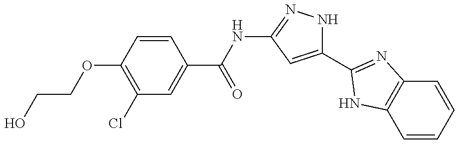 [SMILES: O=C(Nc1cc(-c2nc3ccccc3[nH]2)[nH]n1)c1ccc(OCCO)c(Cl)c1] | N-[5-(1H-benzimidazol-2-yl)-1H-pyrazol- 3-yl]-3-chloro-4-(2-hydroxy- ethoxy)benzamide | A-50 |
| 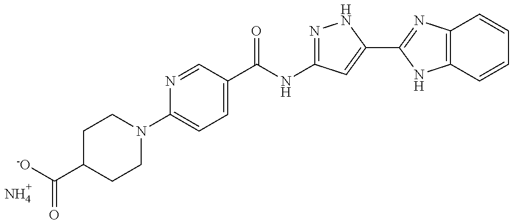 | ammonium 1-[5-[[5-(1H-benzimidazol-2- yl)-1H-pyrazol-3-yl]carbamoyl]-2- pyridyl]piperidine-4-carboxylate | A-51 |
| 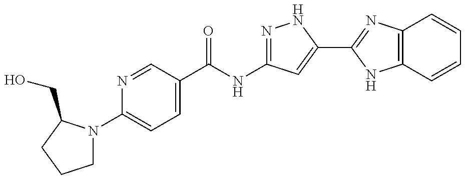 [SMILES: O=C(Nc1cc(-c2nc3ccccc3[nH]2)[nH]n1)c1ccc(N2CCC[C@H]2CO)nc1] | N-[5-(1H-benzimidazol-2-yl)-1H-pyrazol- 3-yl]-6-[(2S)-2-(hydroxymethyl)pyrrolidin- 1-yl]pyridine-3-carboxamide | A-52 |
| 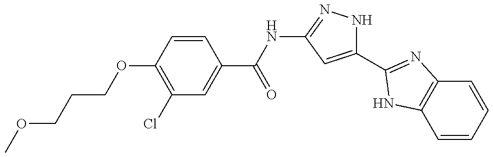 [SMILES: COCCCOc1ccc(C(=O)Nc2cc(-c3nc4ccccc4[nH]3)[nH]n2)cc1Cl] | N-[5-(1H-benzimidazol-2-yl)-1H-pyrazol- 3-yl]-3-chloro-4-(3- methoxypropoxy)benzamide | A-53 |
| 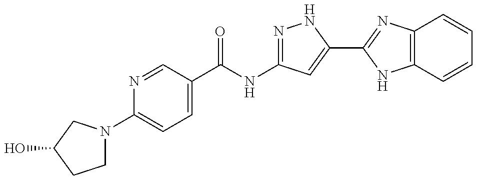 [SMILES: O=C(Nc1cc(-c2nc3ccccc3[nH]2)[nH]n1)c1ccc(N2CC[C@H](O)C2)nc1] | N-[5-(1H-benzimidazol-2-yl)-1H-pyrazol- 3-yl]-6-[(3S)-3-hydroxypyrrolidin-1- yl]pyridine-3-carboxamide | A-54 |
| 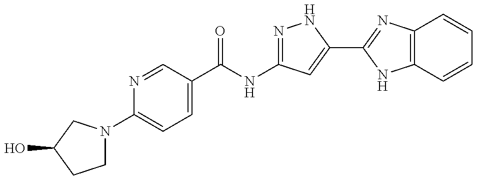 [SMILES: O=C(Nc1cc(-c2nc3ccccc3[nH]2)[nH]n1)c1ccc(N2CC[C@@H](O)C2)nc1] | N-[5-(1H-benzimidazol-2-yl)-1H-pyrazol- 3-yl]-6-[(3R)-3-hydroxypyrrolidin-1- yl]pyridine-3-carboxamide | A-55 |
| 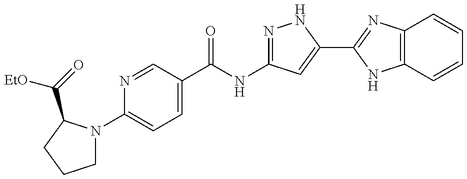 [SMILES: CCOC(=O)[C@@H]1CCCN1c1ccc(C(=O)Nc2cc(-c3nc4ccccc4[nH]3)[nH]n2)cn1] | ethyl (2S)-1-[5-[[5-(1H-benzimidazol-2-yl)- 1H-pyrazol-3-yl]carbamoyl]-2- pyridyl]pyrrolidine-2-carboxylate | A-56 |
| 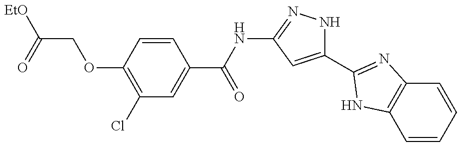 [SMILES: CCOC(=O)COc1ccc(C(=O)Nc2cc(-c3nc4ccccc4[nH]3)[nH]n2)cc1Cl] | ethyl 2-[4-[[5-(1H-benzimidazol-2-yl)-1H- pyrazol-3-yl]carbamoyl]-2-chloro- phenoxy]acetate | A-57 |
| 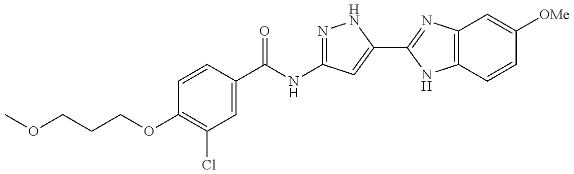 [SMILES: COCCCOc1ccc(C(=O)Nc2cc(-c3nc4cc(OC)ccc4[nH]3)[nH]n2)cc1Cl] | 3-chloro-N-[5-(5-methoxy-1H-benzimi- dazol-2-yl)-1H-pyrazol-3-yl]-4-(3- methoxypropoxy)benzamide | A-58 |
| 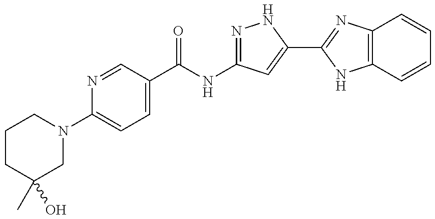 [SMILES: CC1(O)CCCN(c2ccc(C(=O)Nc3cc(-c4nc5ccccc5[nH]4)[nH]n3)cn2)C1] | N-[5-(1H-benzimidazol-2-yl)-1H-pyrazol- 3-yl]-6-(3-hydroxy-3-methyl-1- piperidyl)pyridine-3-carboxamide | A-59 |
| 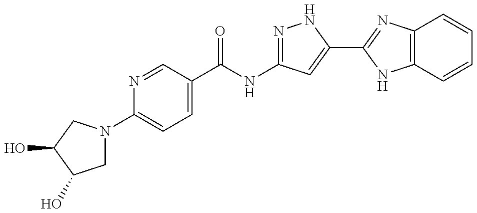 [SMILES: O=C(Nc1cc(-c2nc3ccccc3[nH]2)[nH]n1)c1ccc(N2C[C@H](O)[C@@H](O)C2)nc1] | N-[5-(1H-benzimidazol-2-yl)-1H-pyrazol- 3-yl]-6-[(3S,4S)-3,4-dihydroxypyrrolidin- 1-yl]pyridine-3-carboxamide | A-60 |
| 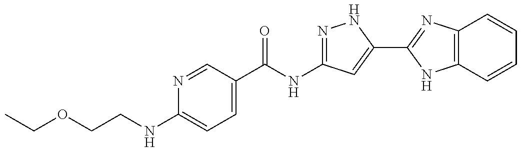 [SMILES: CCOCCNc1ccc(C(=O)Nc2cc(-c3nc4ccccc4[nH]3)[nH]n2)cn1] | N-[5-(1H-benzimidazol-2-yl)-1H-pyrazol- 3-yl]-6-(2-ethoxyethylamino)pyridine-3- carboxamide | A-61 |
| 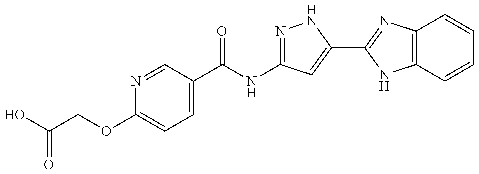 [SMILES: O=C(O)COc1ccc(C(=O)Nc2cc(-c3nc4ccccc4[nH]3)[nH]n2)cn1] | 2-[[5-[[5-(1H-benzimidazol-2-yl)-1H- pyrazol-3-yl]carbamoyl]-2-pyri- dyl]oxy]acetic acid | A-62 |
| 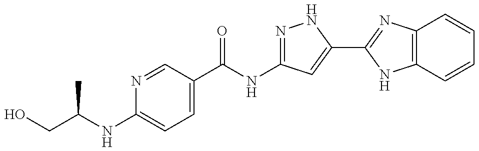 [SMILES: C[C@H](CO)Nc1ccc(C(=O)Nc2cc(-c3nc4ccccc4[nH]3)[nH]n2)cn1] | N-[5-(1H-benzimidazol-2-yl)-1H-pyrazol- 3-yl]-6-[[(1R)-2-hydroxy-1-methyl- ethyl]amino]pyridine-3-carboxamide | A-63 |
| 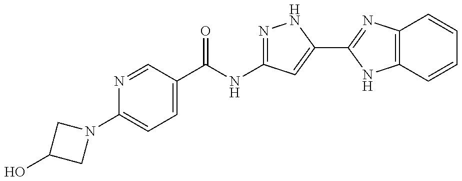 [SMILES: O=C(Nc1cc(-c2nc3ccccc3[nH]2)[nH]n1)c1ccc(N2CC(O)C2)nc1] | N-[5-(1H-benzimidazol-2-yl)-1H-pyrazol- 3-yl]-6-(3-hydroxyazetidin-1-yl)pyridine- 3-carboxamide | A-64 |
| 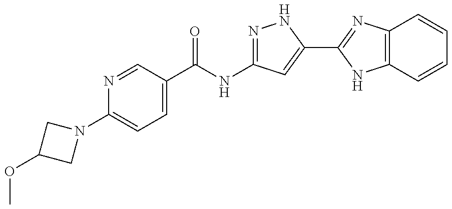 [SMILES: COC1CN(c2ccc(C(=O)Nc3cc(-c4nc5ccccc5[nH]4)[nH]n3)cn2)C1] | N-[5-(1H-benzimidazol-2-yl)-1H-pyrazol- 3-yl]-6-(3-methoxyazetidin-1-yl)pyridine- 3-carboxamide | A-65 |
| 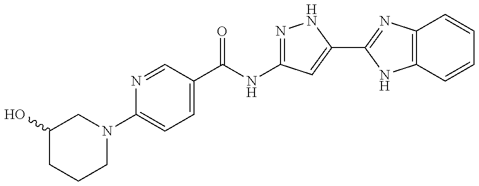 [SMILES: O=C(Nc1cc(-c2nc3ccccc3[nH]2)[nH]n1)c1ccc(N2CCCC(O)C2)nc1] | N-[5-(1H-benzimidazol-2-yl)-1H-pyrazol- 3-yl]-6-(3-hydroxy-1-piperidyl)pyridine-3- carboxamide | A-66 |
| 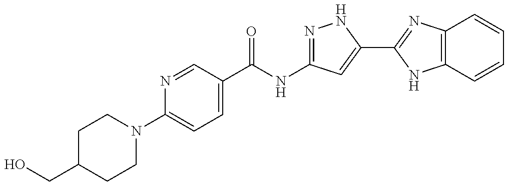 [SMILES: O=C(Nc1cc(-c2nc3ccccc3[nH]2)[nH]n1)c1ccc(N2CCC(CO)CC2)nc1] | N-[5-(1H-benzimidazol-2-yl)-1H-pyrazol- 3-yl]-6-[4-(hydroxymethyl)-1- piperidyl]pyridine-3-carboxamide | A-67 |
| 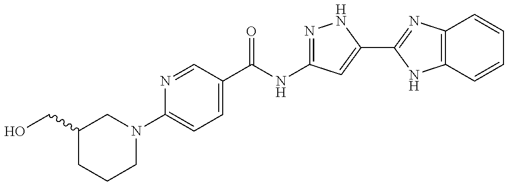 [SMILES: O=C(Nc1cc(-c2nc3ccccc3[nH]2)[nH]n1)c1ccc(N2CCCC(CO)C2)nc1] | N-[5-(1H-benzimidazol-2-yl)-1H-pyrazol- 3-yl]-6-[3-(hydroxymethyl)-1- piperidyl]pyridine-3-carboxamide | A-68 |
| 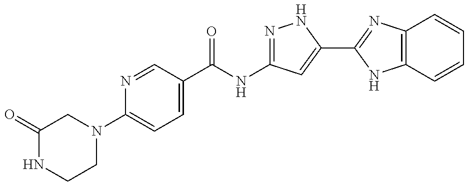 [SMILES: O=C1CN(c2ccc(C(=O)Nc3cc(-c4nc5ccccc5[nH]4)[nH]n3)cn2)CCN1] | N-[5-(1H-benzimidazol-2-yl)-1H-pyrazol- 3-yl]-6-(3-oxopiperazin-1-yl)pyridine-3- carboxamide | A-69 |
| 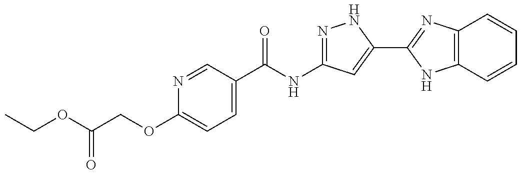 [SMILES: CCOC(=O)COc1ccc(C(=O)Nc2cc(-c3nc4ccccc4[nH]3)[nH]n2)cn1] | ethyl 2-[[5-[[5-(1H-benzimidazol-2-yl)-1H- pyrazol-3-yl]carbamoyl]-2- pyridyl]oxy]acetate | A-70 |
| 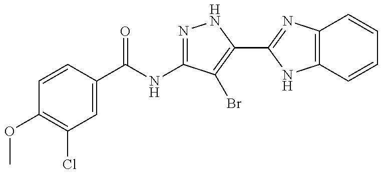 [SMILES: COc1ccc(C(=O)Nc2n[nH]c(-c3nc4ccccc4[nH]3)c2Br)cc1Cl] | N-[5-(1H-benzimidazol-2-yl)-4-bromo-1H- pyrazol-3-yl]-3-chloro-4-methoxy- benzamide | A-71 |
| 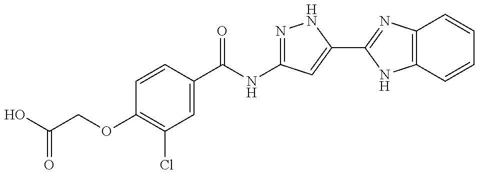 [SMILES: O=C(O)COc1ccc(C(=O)Nc2cc(-c3nc4ccccc4[nH]3)[nH]n2)cc1Cl] | 2-[4-[[5-(1H-benzimidazol-2-yl)-1H- pyrazol-3-yl]carbamoyl]-2-chloro- phenoxy]acetic acid | A-72 |
| 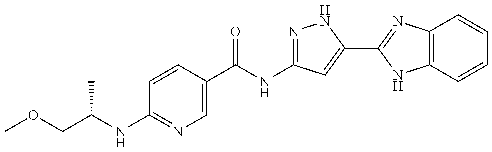 [SMILES: COC[C@H](C)Nc1ccc(C(=O)Nc2cc(-c3nc4ccccc4[nH]3)[nH]n2)cn1] | N-[5-(1H-benzimidazol-2-yl)-1H-pyrazol- 3-yl]-6-[[(1S)-2-methoxy-1-methyl- ethyl]amino]pyridine-3-carboxamide | A-73 |
| 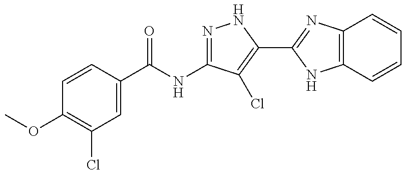 [SMILES: COc1ccc(C(=O)Nc2n[nH]c(-c3nc4ccccc4[nH]3)c2Cl)cc1Cl] | N-[5-(1H-benzimidazol-2-yl)-4-chloro-1H- pyrazol-3-yl]-3-chloro-4-methoxy- benzamide | A-74 |
| 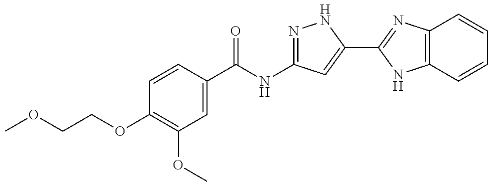 [SMILES: COCCOc1ccc(C(=O)Nc2cc(-c3nc4ccccc4[nH]3)[nH]n2)cc1OC] | N-[5-(1H-benzimidazol-2-yl)-1H-pyrazol- 3-yl]-3-methoxy-4-(2- methoxyethoxy)benzamide | A-75 |
| 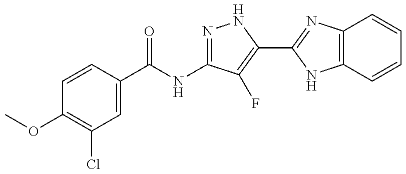 [SMILES: COc1ccc(C(=O)Nc2n[nH]c(-c3nc4ccccc4[nH]3)c2F)cc1Cl] | N-[5-(1H-benzimidazol-2-yl)-4-fluoro-1H- pyrazol-3-yl]-3-chloro-4-methoxy- benzamide | A-76 |
| 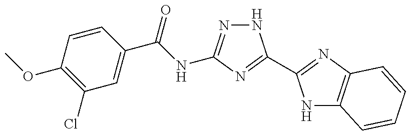 [SMILES: COc1ccc(C(=O)Nc2n[nH]c(-c3nc4ccccc4[nH]3)n2)cc1Cl] | 2-[4-[[5-(1H-benzimidazol-2-yl)-1H- pyrazol-3-yl]carbamoyl]-2-chloro- phenoxy]acetic acid | A-77 |
| 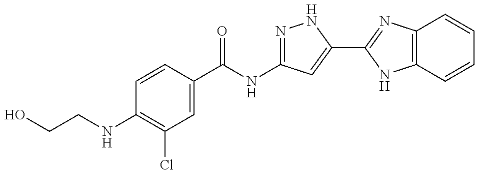 [SMILES: O=C(Nc1cc(-c2nc3ccccc3[nH]2)[nH]n1)c1ccc(NCCO)c(Cl)c1] | N-[5-(1H-benzimidazol-2-yl)-1H-pyrazol- 3-yl]-3-chloro-4-(2- hydroxyethylamino)benzamide | A-78 |
| 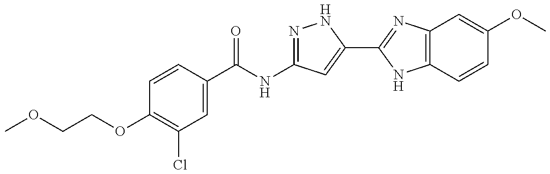 | 3-chloro-N-[5-(5-methoxy-1H-benzimi- dazol-2-yl)-1H-pyrazol-3-yl]-4-(2- methoxethoxy)benzamide | A-79 |
| 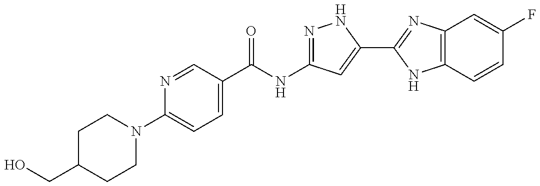 [SMILES: O=C(Nc1cc(-c2nc3cc(F)ccc3[nH]2)[nH]n1)c1ccc(N2CCC(CO)CC2)nc1] | N-[5-(5-fluoro-1H-benzimidazol-2-yl)-1H- pyrazol-3-yl]-6-[4-(hydroxymethyl)-1- piperidyl]pyridine-3-carboxamide | A-80 |
| 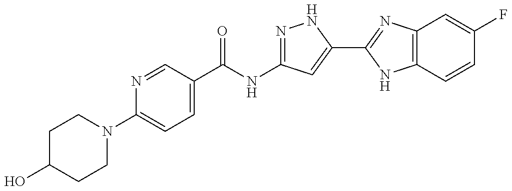 [SMILES: O=C(Nc1cc(-c2nc3cc(F)ccc3[nH]2)[nH]n1)c1ccc(N2CCC(O)CC2)nc1] | N-[5-(5-fluoro-1H-benzimidazol-2-yl)-1H- pyrazol-3-yl]-6-(4-hydroxy-1- piperidyl)pyridine-3-carboxamide | A-81 |
| 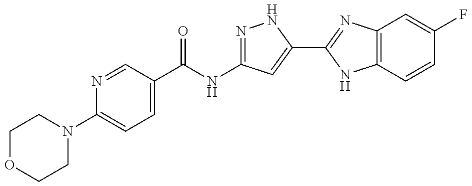 [SMILES: O=C(Nc1cc(-c2nc3cc(F)ccc3[nH]2)[nH]n1)c1ccc(N2CCOCC2)nc1] | N-[5-(5-fluoro-1H-benzimidazol-2-yl)-1H- pyrazol-3-yl]-6-morpholino-pyridine-3- carboxamide | A-82 |
| 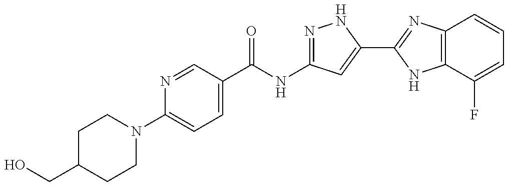 [SMILES: O=C(Nc1cc(-c2nc3cccc(F)c3[nH]2)[nH]n1)c1ccc(N2CCC(CO)CC2)nc1] | N-[5-(7-fluoro-1H-benzimidazol-2-yl)-1H- pyraozl-3-yl]-6-[4-(hydroxymethyl)-1- piperidyl]pyridine-3-carboxamide | A-83 |
| 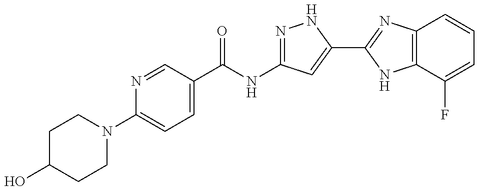 [SMILES: O=C(Nc1cc(-c2nc3cccc(F)c3[nH]2)[nH]n1)c1ccc(N2CCC(O)CC2)nc1] | N-[5-(7-fluoro-1H-benzimidazol-2-yl)-1H- pyrazol-3-yl]-6-[4-(hydroxymethyl)-1- piperidyl]pyridine-3-carboxamide | A-84 |
| 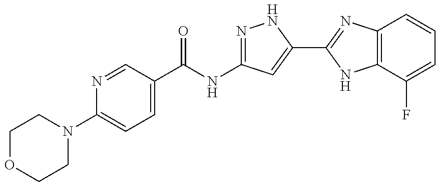 [SMILES: O=C(Nc1cc(-c2nc3cccc(F)c3[nH]2)[nH]n1)c1ccc(N2CCOCC2)nc1] | N-[5-(7-fluoro-1H-benzimidazol-2-yl)-1H- pyrazol-3-yl]-6-morpholino-pyridine-3- carboxamide | A-85 |
| 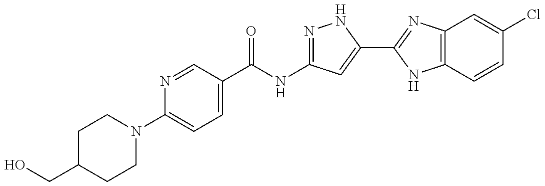 [SMILES: O=C(Nc1cc(-c2nc3cc(Cl)ccc3[nH]2)[nH]n1)c1ccc(N2CCC(CO)CC2)nc1] | N-[5-(5-chloro-1H-benzimidazol-2-yl)-1H- pyrazol-3-yl]-6-[4-(hydroxymethyl)-1- piperidyl]pyridine-3-carboxamide | A-86 |
| 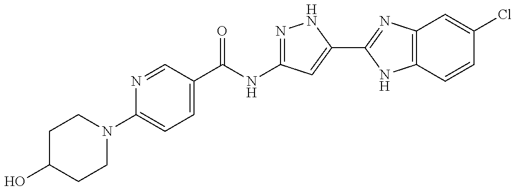 [SMILES: O=C(Nc1cc(-c2nc3cc(Cl)ccc3[nH]2)[nH]n1)c1ccc(N2CCC(O)CC2)nc1] | N-[5-(5-chloro-1H-benzimidazol-2-yl)-1H- pyrazol-3-yl]-6-(4-hydroxy-1- piperidyl)pyridine-3-carboxamide | A-87 |
| 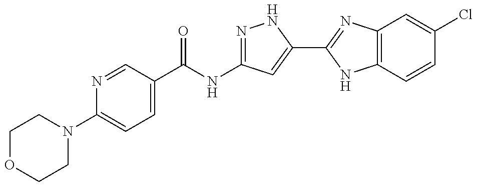 [SMILES: O=C(Nc1cc(-c2nc3cc(Cl)ccc3[nH]2)[nH]n1)c1ccc(N2CCOCC2)nc1] | N-[5-(5-chloro-1H-benzimidazol-2-yl)-1H- pyrazol-3-yl]-6-morpholino-pyridine-3- carboxamide | A-88 |
| 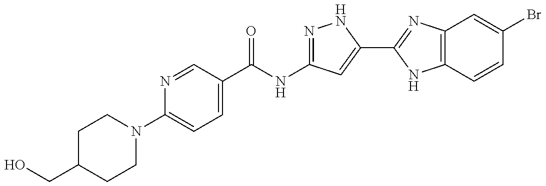 [SMILES: O=C(Nc1cc(-c2nc3cc(Br)ccc3[nH]2)[nH]n1)c1ccc(N2CCC(CO)CC2)nc1] | N-[5-(5-bromo-1H-benzimidazol-2-yl)-1H- pyrazol-3-yl]-6-[4-(hydroxymethyl)-1- piperidyl]pyridine-3-carboxamide | A-89 |
| 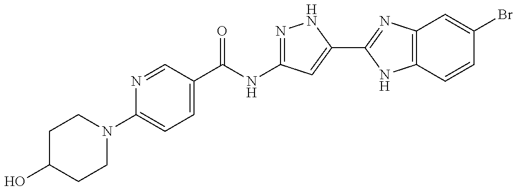 [SMILES: O=C(Nc1cc(-c2nc3cc(Br)ccc3[nH]2)[nH]n1)c1ccc(N2CCC(O)CC2)nc1] | N-[5-(5-bromo-1H-benzimidazol-2-yl)-1H- pyrazol-3-yl]-6-(4-hydroxy-1- piperidyl)pyridine-3-carboxamide | A-90 |
| 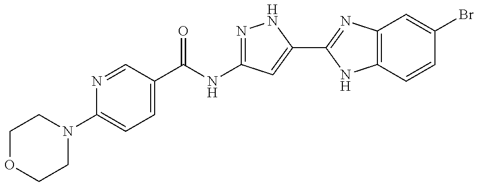 [SMILES: O=C(Nc1cc(-c2nc3cc(Br)ccc3[nH]2)[nH]n1)c1ccc(N2CCOCC2)nc1] | N-[5-(5-bromo-1H-benzimidazol-2-yl)-1H- pyrazol-3-yl]-6-morpholino-pyridine-3- carboxamide | A-91 |
| 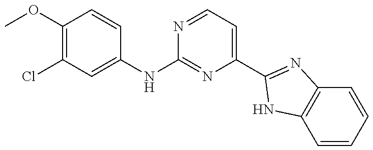 [SMILES: COc1ccc(Nc2nccc(-c3nc4ccccc4[nH]3)n2)cc1Cl] | 4-(1H-benzimidazol-2-yl)-N-(3-chloro-4- methoxy-phenyl)pyrimidin-2-amine | A-92 |
| 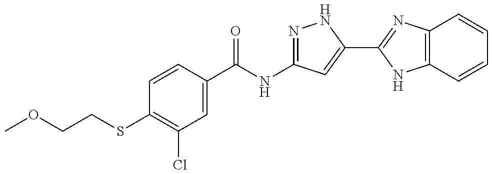 [SMILES: COCCSc1ccc(C(=O)Nc2cc(-c3nc4ccccc4[nH]3)[nH]n2)cc1Cl] | N-[5-(1H-benzimidazol-2-yl)-1H-pyrazol- 3-yl]-3-chloro-4-(2- methoxyethylsulfanyl)benzamide | A-93 |
| 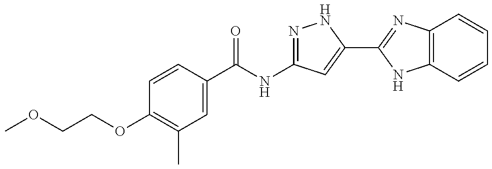 [SMILES: COCCOc1ccc(C(=O)Nc2cc(-c3nc4ccccc4[nH]3)[nH]n2)cc1C] | N-[5-(1H-benzimidazol-2-yl)-1H-pyrazol- 3-yl]-4-(2-methoxyethoxy)-3-methyl- benzamide | A-94 |
| 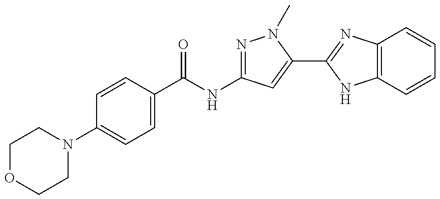 [SMILES: Cn1nc(NC(=O)c2ccc(N3CCOCC3)cc2)cc1-c1nc2ccccc2[nH]1] | N-[5-(1H-benzimidazol-2-yl)-1-methyl- pyrazol-3-yl]-4-morpholino-benzamide | A-95 |
| 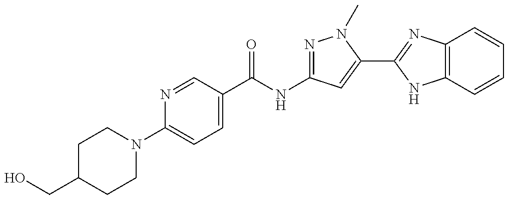 [SMILES: Cn1nc(NC(=O)c2ccc(N3CCC(CO)CC3)nc2)cc1-c1nc2ccccc2[nH]1] | N-[5-(1H-benzimidazol-2-yl)-1-methyl- pyrazol-3-yl]-6-[4-(hydroxymethyl)-1- piperidyl]pyridine-3-carboxamide | A-96 |
| 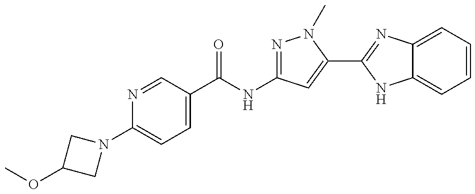 [SMILES: COC1CN(c2ccc(C(=O)Nc3cc(-c4nc5ccccc5[nH]4)n(C)n3)cn2)C1] | N-[5-(1H-benzimidazol-2-yl)-1-methyl- pyrazol-3-yl]-6-(3-methoxyazetidin-1- yl)pyridine-3-carboxamide | A-97 |
| 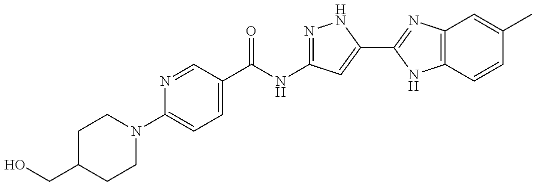 [SMILES: Cc1ccc2[nH]c(-c3cc(NC(=O)c4ccc(N5CCC(CO)CC5)nc4)n[nH]3)nc2c1] | 6-[4-(hydroxymethyl)-1-piperidyl]-N-[5-(5- methyl-1H-benzimidazol-2-yl)-1H-pyrazol- 3-yl]pyridine-3-carboxamide | A-98 |
| 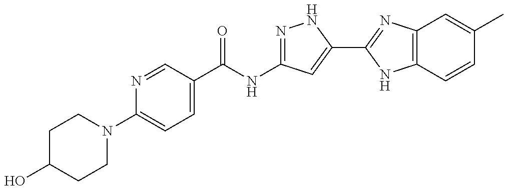 [SMILES: Cc1ccc2[nH]c(-c3cc(NC(=O)c4ccc(N5CCC(O)CC5)nc4)n[nH]3)nc2c1] | 6-(4-hydroxy-1-piperidyl)-N-[5-(5-methyl- 1H-benzimidazol-2-yl)-1H-pyrazol-3- yl]pyridine-3-carboxamide | A-99 |
| 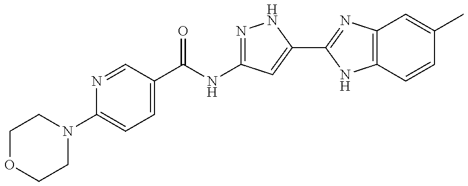 [SMILES: Cc1ccc2[nH]c(-c3cc(NC(=O)c4ccc(N5CCOCC5)nc4)n[nH]3)nc2c1] | N-[5-(5-methyl-1H-benzimidazol-2-yl)-1H- pyrazol-3-yl]-6-morpholino-pyridine-3- carboxamide | A-100 |
| 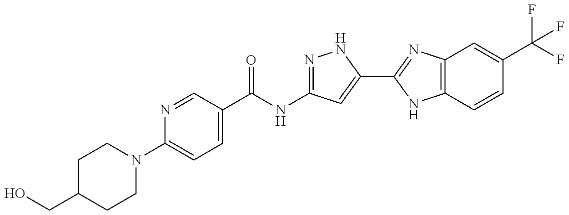 [SMILES: O=C(Nc1cc(-c2nc3cc(C(F)(F)F)ccc3[nH]2)[nH]n1)c1ccc(N2CCC(CO)CC2)nc1] | 6-[4-(hydroxymethyl)-1-piperidyl]-N-[5-[5- (trifluoromethyl)-1H-benzimidazol-2-yl]- 1H-pyrazol-3-yl]pyridine-3-carboxamide | A-101 |
| 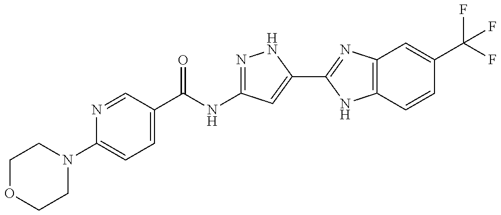 [SMILES: O=C(Nc1cc(-c2nc3cc(C(F)(F)F)ccc3[nH]2)[nH]n1)c1ccc(N2CCOCC2)nc1] | 6-morpholino-N-[5-[5-(trifluoromethyl)- 1H-benzimidazol-2-yl]-1H-pyrazol-3- yl]pyridine-3-carboxamide | A-102 |
| 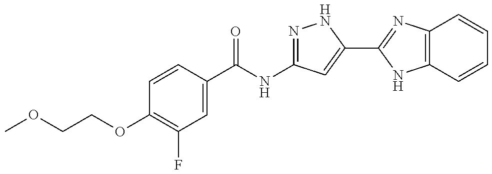 [SMILES: COCCOc1ccc(C(=O)Nc2cc(-c3nc4ccccc4[nH]3)[nH]n2)cc1F] | N-[5-(1H-benzimidazol-2-yl)-1H-pyrazol- 3-yl]-3-fluoro-4-(2-methoxy- ethoxy)benzamide | A-103 |
| 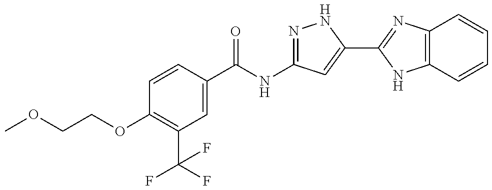 [SMILES: COCCOc1ccc(C(=O)Nc2cc(-c3nc4ccccc4[nH]3)[nH]n2)cc1C(F)(F)F] | N-[5-(1H-benzimidazol-2-yl)-1H-pyrazol- 3-yl]-4-(2-methoxyethoxy)-3- (trifluoromethyl)benzamide | A-104 |
| 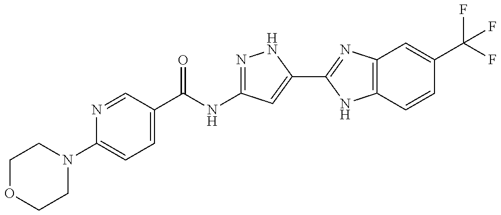 [SMILES: O=C(Nc1cc(-c2nc3cc(C(F)(F)F)ccc3[nH]2)[nH]n1)c1ccc(N2CCOCC2)nc1] | 6-(4-hydroxy-1-piperidyl)-N-[5-[5- (trifluoromethyl)-1H-benzimidazol-2-yl]- 1H-pyrazol-3-yl]pyridine-3-carboxamide | A-105 |
| 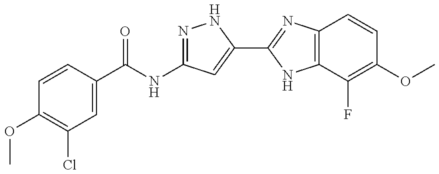 [SMILES: COc1ccc(C(=O)Nc2cc(-c3nc4ccc(OC)c(F)c4[nH]3)[nH]n2)cc1Cl] | 3-chloro-N-[5-(7-fluoro-6-methoxy-1H- benzimidazol-2-yl)-1H-pyrazol-3-yl]-4- methoxy-benzamide | A-106 |
| 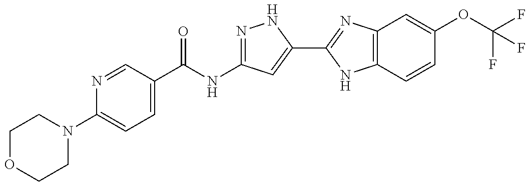 [SMILES: O=C(Nc1cc(-c2nc3cc(OC(F)(F)F)ccc3[nH]2)[nH]n1)c1ccc(N2CCOCC2)nc1] | 6-morpholino-N-[5-[5-(trifluoromethoxy)- 1H-benzimidazol-2-yl]-1H-pyrazol-3- yl]pyridine-3-carboxamide | A-107 |
| 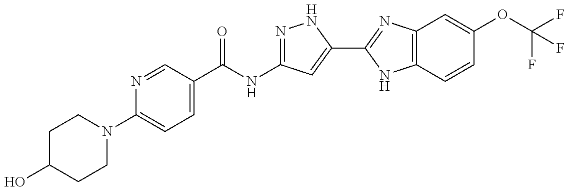 [SMILES: O=C(Nc1cc(-c2nc3cc(OC(F)(F)F)ccc3[nH]2)[nH]n1)c1ccc(N2CCC(O)CC2)nc1] | 6-(4-hydroxy-1-piperidyl)-N-[5-[5- (trifluoromethoxy)-1H-benzimidazol-2-yl]- 1H-pyraozl-3-yl]pyridine-3-carboxamide | A-108 |
| 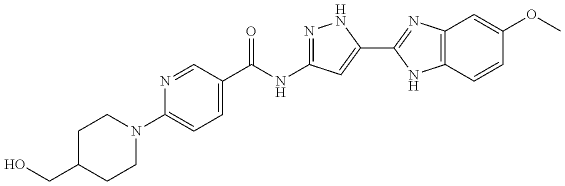 [SMILES: COc1ccc2[nH]c(-c3cc(NC(=O)c4ccc(N5CCC(CO)CC5)nc4)n[nH]3)nc2c1] | 6-[4-(hydroxymethyl)-1-piperidyl]-N-[5-(5- methoxy-1H-benzimidazol-2-yl)-1H- pyrazol-3-yl]pyridine-3-carboxamide | A-109 |
| 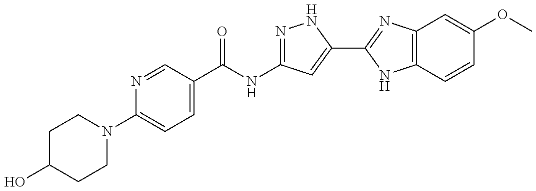 [SMILES: COc1ccc2[nH]c(-c3cc(NC(=O)c4ccc(N5CCC(O)CC5)nc4)n[nH]3)nc2c1] | 6-(4-hydroxy-1-piperidyl)-N-[5-(5- methoxy-1H-benzimidazol-2-yl)-1H- pyrazol-3-yl]pyridine-3-carboxamide | A-110 |
| 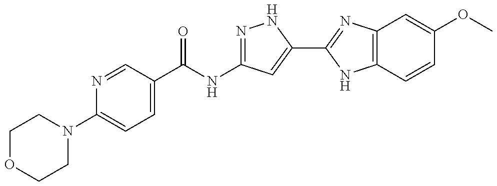 [SMILES: COc1ccc2[nH]c(-c3cc(NC(=O)c4ccc(N5CCOCC5)nc4)n[nH]3)nc2c1] | N-[5-(5-methoxy-1H-benzimidazol-2-yl)- 1H-pyrazol-3-yl]-6-morpholino-pyridine-3- carboxamide | A-111 |
| 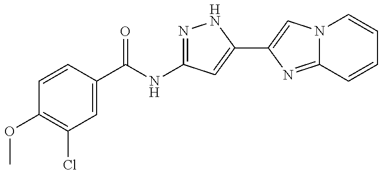 [SMILES: COc1ccc(C(=O)Nc2cc(-c3cn4ccccc4n3)[nH]n2)cc1Cl] | 3-chloro-N-(5-imidazo[1,2-a]pyridin-2-yl- 1H-pyrazol-3-yl)-4-methoxy-benzamide | A-112 |
| 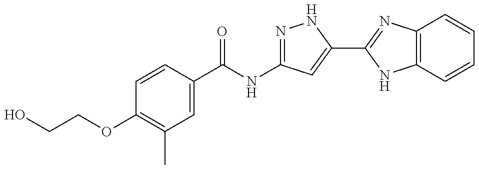 [SMILES: Cc1cc(C(=O)Nc2cc(-c3nc4ccccc4[nH]3)[nH]n2)ccc1OCCO] | N-[5-(1H-benzimidazol-2-yl)-1H-pyrazol- 3-yl]-4-(2-hydroxyethoxy)-3-methyl- benzamide | A-113 |
| 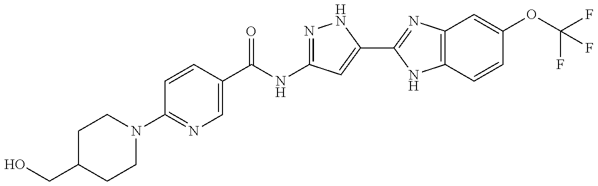 [SMILES: O=C(Nc1cc(-c2nc3cc(OC(F)(F)F)ccc3[nH]2)[nH]n1)c1ccc(N2CCC(CO)CC2)nc1] | 6-[4-(hydroxymethyl)-1-piperidyl]-N-[5-[5- (trifluoromethoxy)-1H-benzimidazol-2-yl]- 1H-pyrazol-3-yl]pyridine-3-carboxamide | A-114 |
| 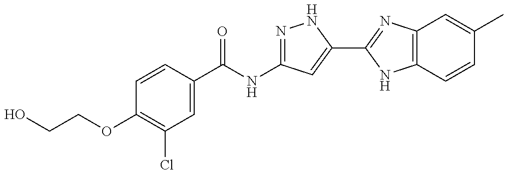 [SMILES: Cc1ccc2[nH]c(-c3cc(NC(=O)c4ccc(OCCO)c(Cl)c4)n[nH]3)nc2c1] | 3-chloro-4-(2-hydroxyethoxy)-N-[5-(5- methyl-1H-benzimidazol-2-yl)-1H-pyrazol- 3-yl]benzamide | A-115 |
| 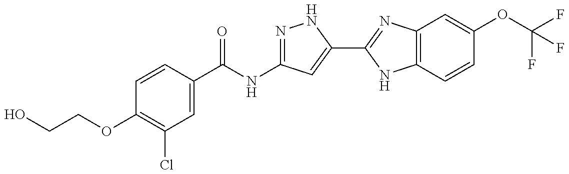 [SMILES: O=C(Nc1cc(-c2nc3cc(OC(F)(F)F)ccc3[nH]2)[nH]n1)c1ccc(OCCO)c(Cl)c1] | 3-chloro-4-(2-hydroxyethoxy)-N-[5-[5- (trifluoromethoxy)-1H-benzimidazol-2-yl]- 1H-pyrazol-3-yl]benzamide | A-116 |
| 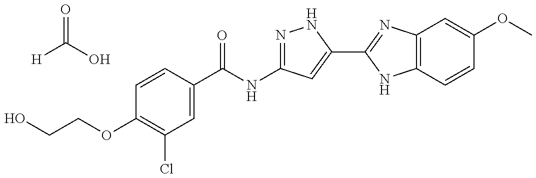 [SMILES: COc1ccc2[nH]c(-c3cc(NC(=O)c4ccc(OCCO)c(Cl)c4)n[nH]3)nc2c1 ; O=CO] | 3-chloro-4-(2-hydroxyethoxy)-N-[5-(5- methoxy-1H-benzimidazol-2-yl)-1H- pyrazol-3-yl]benzamide formic acid | A-117 |
| 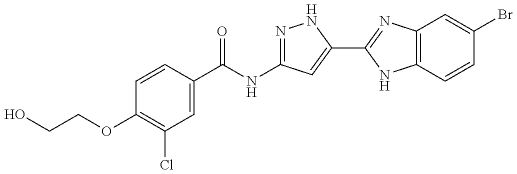 [SMILES: O=C(Nc1cc(-c2nc3cc(Br)ccc3[nH]2)[nH]n1)c1ccc(OCCO)c(Cl)c1] | N-[5-(5-bromo-1H-benzimidazol-2-yl)-1H- pyrazol-3-yl]-3-chloro-4-(2- hydroxyethoxy)benzamide | A-118 |
| 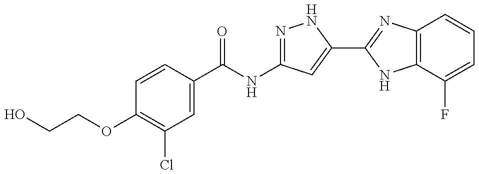 [SMILES: O=C(Nc1cc(-c2nc3cccc(F)c3[nH]2)[nH]n1)c1ccc(OCCO)c(Cl)c1] | 3-chloro-N-[5-(7-fluoro-1H-benzimidazol- 2-yl)-1H-pyrazol-3-yl]-4-(2- hydroxyethoxy)bezamide | A-119 |
| 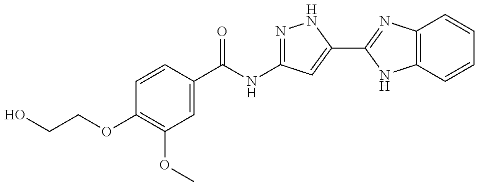 [SMILES: COc1cc(C(=O)Nc2cc(-c3nc4ccccc4[nH]3)[nH]n2)ccc1OCCO] | N-[5-(1H-benzimidazol-2-yl)-1H-pyrazol- 3-yl]-4-(2-hydroxyethoxy)-3-methoxy- benzamide | A-120 |
| 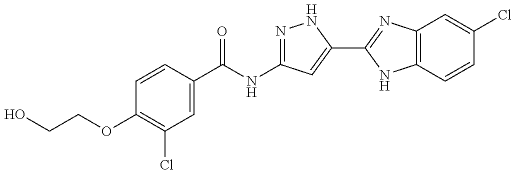 [SMILES: O=C(Nc1cc(-c2nc3cc(Cl)ccc3[nH]2)[nH]n1)c1ccc(OCCO)c(Cl)c1] | 3-chloro-N-[5-(5-chloro-1H-benzimidazol- 2-yl)-1H-pyrazol-3-yl]-4-(2- hydroxyethoxy)benzamide | A-121 |
| 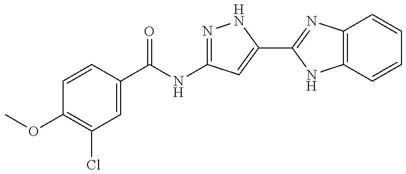 [SMILES: COc1ccc(C(=O)Nc2cc(-c3nc4ccccc4[nH]3)[nH]n2)cc1Cl] | N-[5-(1H-benzimidazol-2-yl)-4-methyl-1H- pyrazol-3-yl]-3-chloro-4-methoxy- benzamide | A-122 |
| 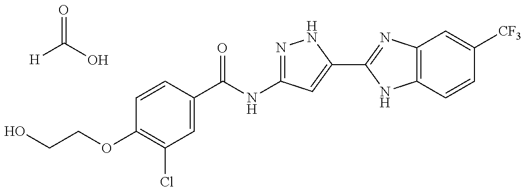 [SMILES: O=C(Nc1cc(-c2nc3cc(C(F)(F)F)ccc3[nH]2)[nH]n1)c1ccc(OCCO)c(Cl)c1 ; O=CO] | 3-chloro-4-(2-hydroxyethoxy)-N-[5-[5- (trifluoromethyl)-1H-benzimidazol-2-yl]- 1H-pyrazol-3-yl]benzamide formic acid | A-123 |
| 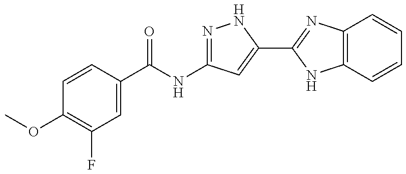 [SMILES: COc1ccc(C(=O)Nc2cc(-c3nc4ccccc4[nH]3)[nH]n2)cc1F] | N-[5-(1H-benzimidazol-2-yl)-1H-pyrazol- 3-yl]-3-fluoro-4-methoxy-benzamide | A-124 |
| 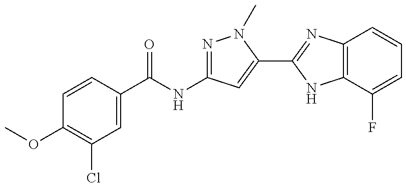 [SMILES: COc1ccc(C(=O)Nc2cc(-c3nc4cccc(F)c4[nH]3)n(C)n2)cc1Cl] | 3-chloro-N-[5-(7-fluoro-1H-benzimidazol- 2-yl)-1-methyl-pyrazol-3-yl]-4-methoxy- benzamide | A-125 |
| 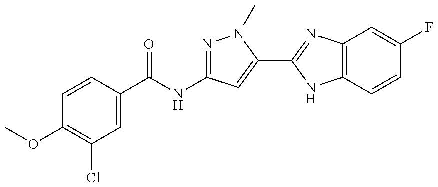 [SMILES: COc1ccc(C(=O)Nc2cc(-c3nc4cc(F)ccc4[nH]3)n(C)n2)cc1Cl] | 3-chloro-N-[5-(5-fluoro-1H-benzimidazol- 2-yl)-1-methyl-pyrazol-3-yl]-4-methoxy- benzamide | A-126 |
| 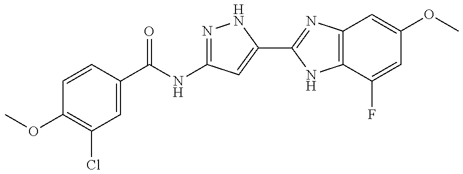 | 3-chloro-N-[5-(7-fluoro-5-methoxy-1H- benzimidazol-2-yl)-1H-pyrazol-3-yl]-4- methoxy-benzamide | A-127 |
| 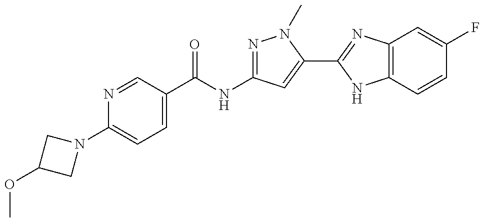 [SMILES: COC1CN(c2ccc(C(=O)Nc3cc(-c4nc5cc(F)ccc5[nH]4)n(C)n3)cn2)C1] | N-[5-(5-fluoro-1H-benzimidazol-2-yl)-1- methyl-pyrazol-3-yl]-6-(3-methoxyazetidin- 1-yl)pyridine-3-carboxamide | A-128 |
| 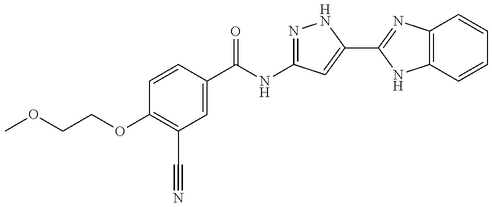 [SMILES: COCCOc1ccc(C(=O)Nc2cc(-c3nc4ccccc4[nH]3)[nH]n2)cc1C#N] | N-[5-(1H-benzimidazol-2-yl)-1H-pyrazol- 3-yl]-3-cyano-4-(2-methoxy- ethoxy)benzamide | A-129 |
| 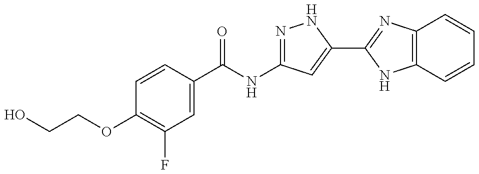 [SMILES: O=C(Nc1cc(-c2nc3ccccc3[nH]2)[nH]n1)c1ccc(OCCO)c(F)c1] | N-[5-(1H-benzimidazol-2-yl)-1H-pyrazol- 3-yl]-3-fluoro-4-(2-hydroxy- ethoxy)benzamide | A-130 |
| 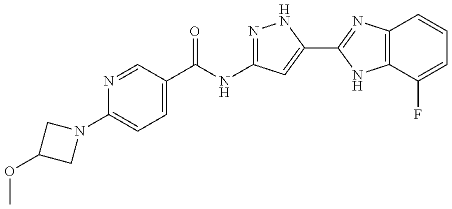 [SMILES: COC1CN(c2ccc(C(=O)Nc3cc(-c4nc5cccc(F)c5[nH]4)[nH]n3)cn2)C1] | N-[5-(7-fluoro-1H-benzimidazol-2-yl)-1H- pyrazol-3-yl]-6-(3-methoxyazetidin-1- yl)pyridine-3-carboxamide | A-131 |
| 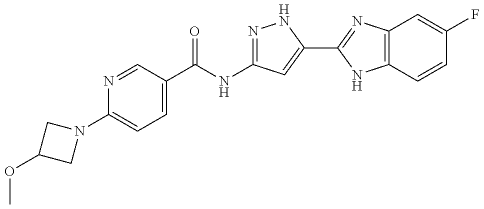 [SMILES: COC1CN(c2ccc(C(=O)Nc3cc(-c4nc5cc(F)ccc5[nH]4)[nH]n3)cn2)C1] | N-[5-(5-fluoro-1H-benzimidazol-2-yl)-1H- pyrazol-3-yl]-6-(3-methoxyazetidin-1- yl)pyridine-3-carboxamide | A-132 |
| 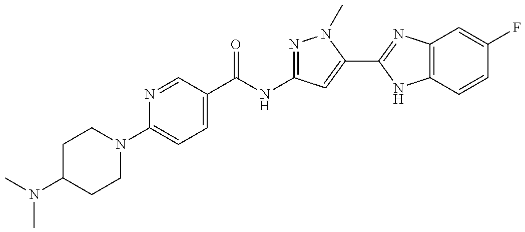 [SMILES: CN(C)C1CCN(c2ccc(C(=O)Nc3cc(-c4nc5cc(F)ccc5[nH]4)n(C)n3)cn2)CC1] | 6-[4-(dimethylamino)-1-piperidyl]-N-[5-(5- fluoro-1H-benzimidazol-2-yl)-1-methyl- pyraozl-3-yl]pyridine-3-carobxamide | A-133 |
| 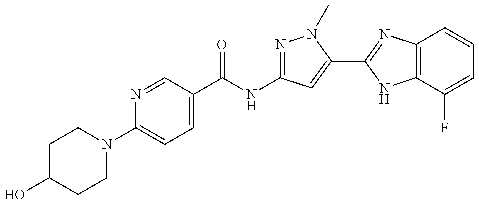 [SMILES: Cn1nc(NC(=O)c2ccc(N3CCC(O)CC3)nc2)cc1-c1nc2cccc(F)c2[nH]1] | N-[5-(4-fluoro-1H-benzimidazol-2-yl)-1- methyl-pyrazol-3-yl]-6-(4-hydroxy-1- piperidyl)pyridine-3-carboxamide | A-134 |
| 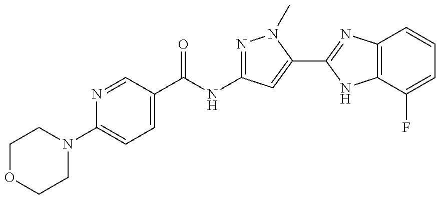 [SMILES: Cn1nc(NC(=O)c2ccc(N3CCOCC3)nc2)cc1-c1nc2cccc(F)c2[nH]1] | N-[5-(4-fluoro-1H-benzimidazol-2-yl)-1- methyl-pyrazol-3-yl]-6-morpholino- pyridine-3-carboxamide | A-135 |
| 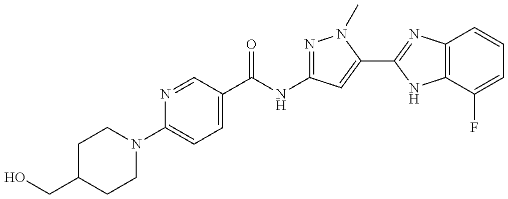 [SMILES: Cn1nc(NC(=O)c2ccc(N3CCC(CO)CC3)nc2)cc1-c1nc2cccc(F)c2[nH]1] | N-[5-(4-fluoro-1H-benzimidazol-2-yl)-1- methyl-pyrazol-3-yl]-6-[4-(hydroxymethyl)- 1-piperidyl]pyridine-3-carboxamide | A-136 |
| 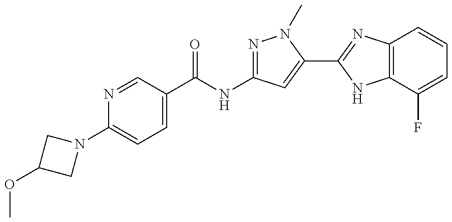 [SMILES: COC1CN(c2ccc(C(=O)Nc3cc(-c4nc5cccc(F)c5[nH]4)n(C)n3)cn2)C1] | N-[5-(4-fluoro-1H-benzimidazol-2-yl)-1- methyl-pyrazol-3-yl]-6-(3-methoxyazetidin- 1-yl)pyridine-3-carboxamide | A-137 |
| 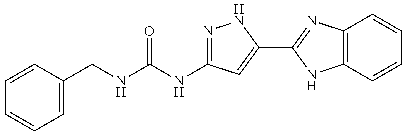 [SMILES: O=C(NCc1ccccc1)Nc1cc(-c2nc3ccccc3[nH]2)[nH]n1] | 1-[5-(1H-benzimidazol-2-yl)-1H-pyrazol-3- yl]-3-benzyl-urea | A-138 |
| 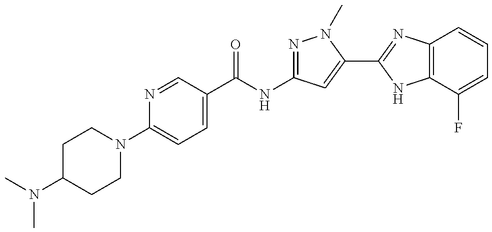 [SMILES: CN(C)C1CCN(c2ccc(C(=O)Nc3cc(-c4nc5cccc(F)c5[nH]4)n(C)n3)cn2)CC1] | 6-[4-(dimethylamino)-1-piperidyl]-N-[5-(4- fluoro-1H-benzimidazol-2-yl)-1-methyl- pyrazol-3-yl]pyridine-3-carboxamide | A-139 |
| 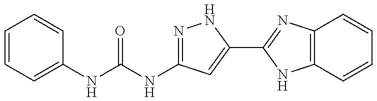 [SMILES: O=C(Nc1ccccc1)Nc1cc(-c2nc3ccccc3[nH]2)[nH]n1] | 1-[5-(1H-benzimidazol-2-yl)-1H-pyrazol-3- yl]-3-phenyl-urea | A-140 |
| 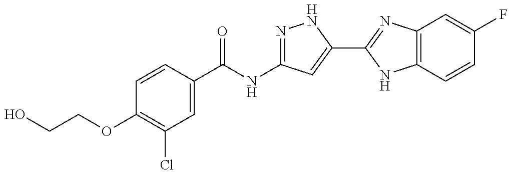 [SMILES: O=C(Nc1cc(-c2nc3cc(F)ccc3[nH]2)[nH]n1)c1ccc(OCCO)c(Cl)c1] | 3-chloro-N-[5-(5-fluoro-1H-benzimidazol- 2-yl)-1H-pyrazol-3-yl]-4-(2- hydroxyethoxy)benzamide | A-141 |
| 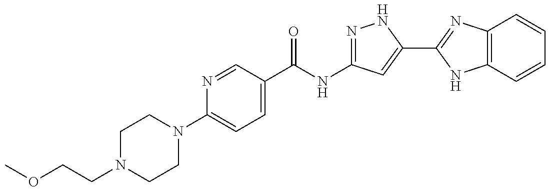 [SMILES: COCCN1CCN(c2ccc(C(=O)Nc3cc(-c4nc5ccccc5[nH]4)[nH]n3)cn2)CC1] | N-[5-(1H-benzimidazol-2-yl)-1H-pyrazol-3- yl]-6-[4-(2-methoxyethyl)piperazin-1- yl]pyridine-3-carboxamide | A-142 |
| 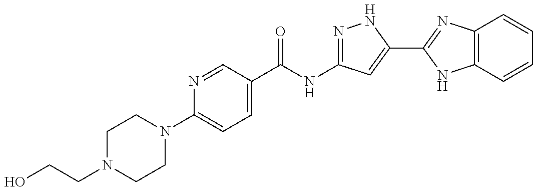 [SMILES: O=C(Nc1cc(-c2nc3ccccc3[nH]2)[nH]n1)c1ccc(N2CCN(CCO)CC2)nc1] | N-[5-(1H-benzimidazol-2-yl)-1H-pyrazol-3- yl]-6-[4-(2-hydroxyethyl)piperazin-1- yl]pyridine-3-carboxamide | A-143 |
| 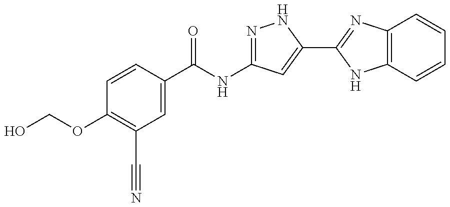 [SMILES: N#Cc1cc(C(=O)Nc2cc(-c3nc4ccccc4[nH]3)[nH]n2)ccc1OCO] | N-[5-(1H-benzimidazol-2-yl)-1H-pyrazol- 3-yl]-3-cyano-4-(2-hydroxy- ethoxy)benzamide | A-144 |
| 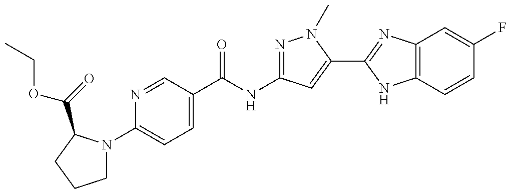 [SMILES: CCOC(=O)[C@@H]1CCCN1c1ccc(C(=O)Nc2cc(-c3nc4cc(F)ccc4[nH]3)n(C)n2)cn1] | ethyl (2S)-1-[5-[[5-(5-fluoro-1H- benzimidazol-2-yl)-1-methyl-pyrazol-3- yl]carbamoyl]-2-pyridyl]pyrrolidine-2- carboxylate | A-145 |
| 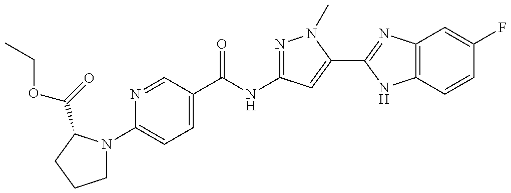 [SMILES: CCOC(=O)[C@H]1CCCN1c1ccc(C(=O)Nc2cc(-c3nc4cc(F)ccc4[nH]3)n(C)n2)cn1] | ethyl (2R)-1-[5-[[5-(5-fluoro-1H- benzimidazol-2-yl)-1-methyl-pyrazol-3- yl]carbamoyl]-2-pyridyl]pyrrolidine-2- carboxylate | A-146 |
| 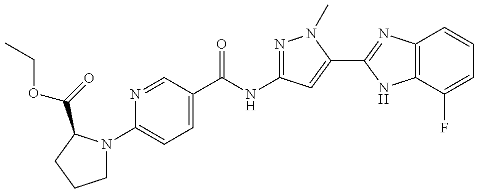 [SMILES: CCOC(=O)[C@@H]1CCCN1c1ccc(C(=O)Nc2cc(-c3nc4cccc(F)c4[nH]3)n(C)n2)cn1] | ethyl (2S)-1-[5-[[5-(7-fluoro-1H- benzimidazol-2-yl)-1-methyl-pyrazol-3- yl]carbamoyl]-2-pyridyl]pyrrolidine-2- carboxylate | A-147 |
| 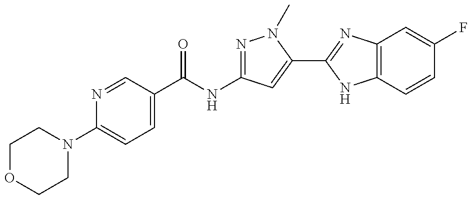 [SMILES: Cn1nc(NC(=O)c2ccc(N3CCOCC3)nc2)cc1-c1nc2cc(F)ccc2[nH]1] | N-[5-(5-fluoro-1H-benzimidazol-2-yl)-1- methyl-pyrazol-3-yl]-6-morpholino- pyridine-3-carboxamide | A-148 |
| 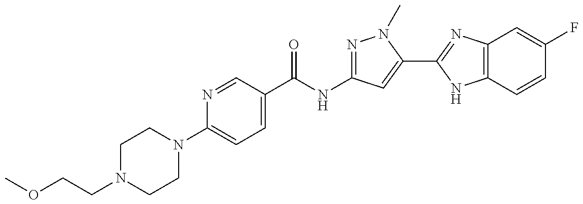 [SMILES: COCCN1CCN(c2ccc(C(=O)Nc3cc(-c4nc5cc(F)ccc5[nH]4)n(C)n3)cn2)CC1] | N-[5-(5-fluoro-1H-benzimidazol-2-yl)-1- methyl-pyrazol-3-yl]-6-[4-(2- methoxyethyl)piperazin-1-yl]pyridine-3- carboxamide | A-149 |
| 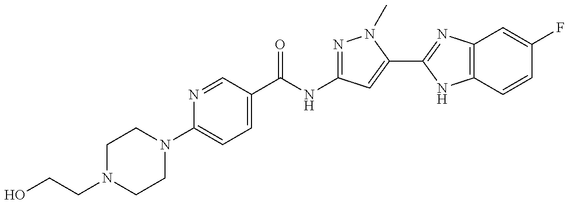 [SMILES: Cn1nc(NC(=O)c2ccc(N3CCN(CCO)CC3)nc2)cc1-c1nc2cc(F)ccc2[nH]1] | N-[5-(5-fluoro-1H-benzimidazol-2-yl)-1- methyl-pyrazol-3-yl]-6-[4-(2- hydroxyethyl)piperazin-1-yl]pyridine-3- carboxamide | A-150 |
| 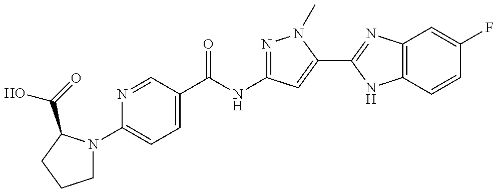 [SMILES: Cn1nc(NC(=O)c2ccc(N3CCC[C@H]3C(=O)O)nc2)cc1-c1nc2cc(F)ccc2[nH]1] | (2S)-1-[5-[[5-(5-fluoro-1H-benzimidazol-2- yl)-1-methyl-pyrazol-3-yl]carbamoyl]-2- pyridyl]pyrrolidine-2-carboxylic acid | A-151 |
| 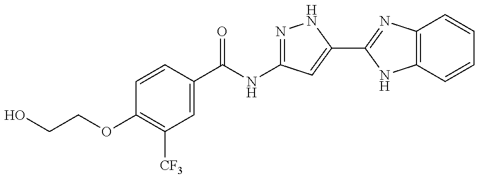 [SMILES: O=C(Nc1cc(-c2nc3ccccc3[nH]2)[nH]n1)c1ccc(OCCO)c(C(F)(F)F)c1] | N-[5-(1H-benzimidazol-2-yl)-1H-pyrazol- 3-yl]-4-(2-hydroxyethoxy)-3- (trifluoromethyl)benzamide | A-152 |
| 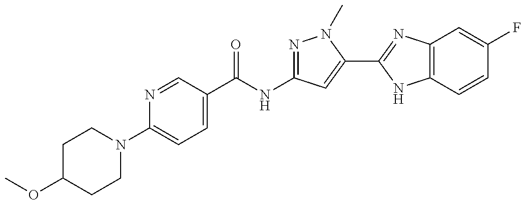 [SMILES: COC1CCN(c2ccc(C(=O)Nc3cc(-c4nc5cc(F)ccc5[nH]4)n(C)n3)cn2)CC1] | N-[5-(5-fluoro-1H-benzimidazol-2-yl)-1- methyl-pyrazol-3-yl]-6-(4-methoxy-1- piperidyl)pyridine-3-carboxamide | A-153 |
| 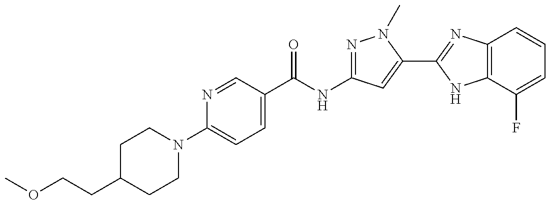 [SMILES: COCCC1CCN(c2ccc(C(=O)Nc3cc(-c4nc5cccc(F)c5[nH]4)n(C)n3)cn2)CC1] | N-[5-(7-fluoro-1H-benzimidazol-2-yl)-1- methyl-pyrazol-3-yl]-6-[4-(2- methoxyethyl)piperazin-1-yl]pyridine-3- carboxamide | A-154 |
| 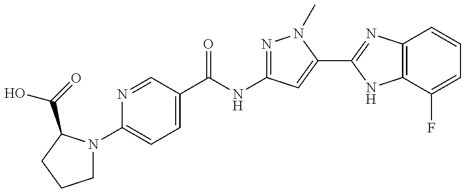 [SMILES: Cn1nc(NC(=O)c2ccc(N3CCC[C@H]3C(=O)O)nc2)cc1-c1nc2cccc(F)c2[nH]1] | (2S)-1-[5-[[5-(7-fluoro-1H-benzimidazol-2- yl)-1-methyl-pyrazol-3-yl]carbamoyl]-2- pyridyl]pyrrolidine-2-carboxylic acid | A-155 |
| 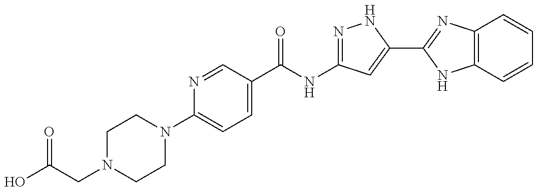 [SMILES: O=C(O)CN1CCN(c2ccc(C(=O)Nc3cc(-c4nc5ccccc5[nH]4)[nH]n3)cn2)CC1] | 2-[4-[5-[[5-(1H-benzimidazol-2-yl)-1H- pyrazol-3-yl]carbamoyl]-2-pyri- dyl]piperazin-1-yl]acetic acid | A-156 |
| 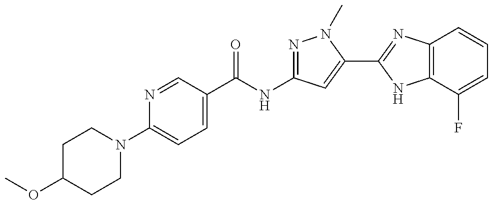 [SMILES: COC1CCN(c2ccc(C(=O)Nc3cc(-c4nc5cccc(F)c5[nH]4)n(C)n3)cn2)CC1] | N-[5-(7-fluoro-1H-benzimidazol-2-yl)-1- methyl-pyrazol-3-yl]-6-(4-methoxy-1- piperidyl)pyridine-3-carboxamide | A-157 |
| 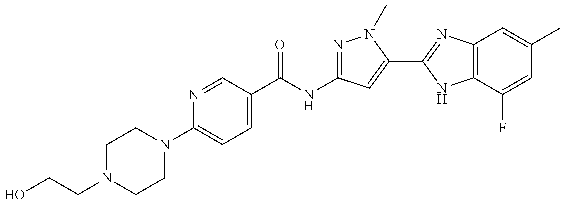 [SMILES: Cc1cc(F)c2[nH]c(-c3cc(NC(=O)c4ccc(N5CCN(CCO)CC5)nc4)nn3C)nc2c1] | N-[5-(7-fluoro-1H-benzimidazol-2-yl)-1- methyl-pyrazol-3-yl]-6-[4-(2- hydroxyethyl)piperazin-1-yl]pyridine-3- carboxamide | A-158 |
| 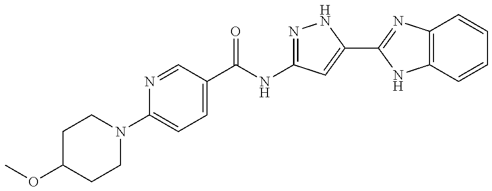 [SMILES: COC1CCN(c2ccc(C(=O)Nc3cc(-c4nc5ccccc5[nH]4)[nH]n3)cn2)CC1] | N-[5-(1H-benzimidazol-2-yl)-1H-pyrazol-3- yl]-6-(4-methoxy-1-piperidyl)pyridine-3- carboxamide | A-159 |
| 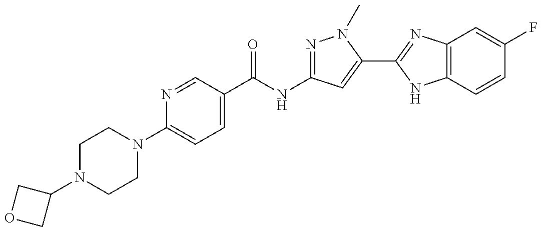 [SMILES: Cn1nc(NC(=O)c2ccc(N3CCN(C4COC4)CC3)nc2)cc1-c1nc2cc(F)ccc2[nH]1] | N-[5-(5-fluoro-1H-benzimidazol-2-yl)-1- methyl-pyrazol-3-yl]-6-[4-(oxetan-3- yl)piperazin-1-yl]pyridine-3-carboxamide | A-160 |
| 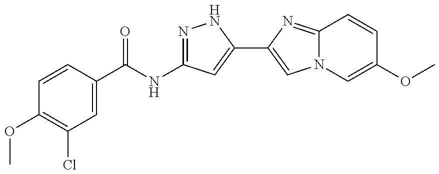 [SMILES: COc1ccc2nc(-c3cc(NC(=O)c4ccc(OC)c(Cl)c4)n[nH]3)cn2c1] | 3-chloro-4-methoxy-N-[5-(6- methoxyimidazo[1,4-a]pyridin-2-yl)-1H- pyrazol-3-yl]benzamide | A-161 |
| 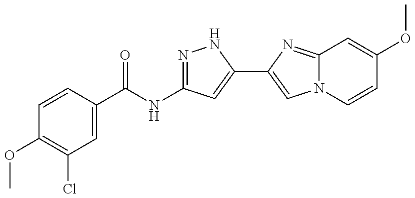 [SMILES: COc1ccn2cc(-c3cc(NC(=O)c4ccc(OC)c(Cl)c4)n[nH]3)nc2c1] | 3-chloro-4-methoxy-N-[5-(7- methoxyimidazo[1,2-a]pyridin-2-yl)-1H- pyrazol-3-yl]benzamide | A-162 |
| 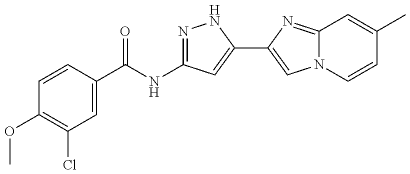 [SMILES: COc1ccc(C(=O)Nc2cc(-c3cn4ccc(C)cc4n3)[nH]n2)cc1Cl] | 3-chloro-4-methoxy-N-[5-(7- methylimidazo[1,2-a]pyridin-2-yl)-1H- pyrazol-3-yl]benzamide | A-163 |
| 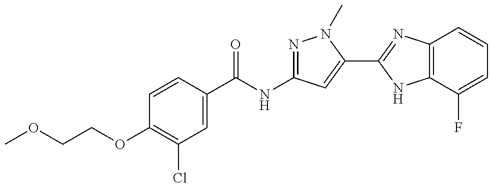 [SMILES: COCCOc1ccc(C(=O)Nc2cc(-c3nc4cccc(F)c4[nH]3)n(C)n2)cc1Cl] | 3-chloro-N-[5-(7-fluoro-1H-benzimidazol- 2-yl)-1-methyl-pyrazol-3-yl]-4-(2- methoxyethoxy)benzamide | A-164 |
| 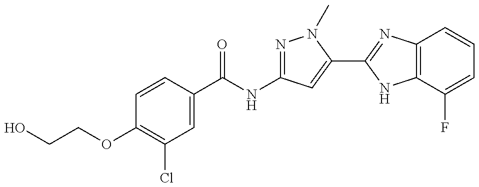 [SMILES: Cn1nc(NC(=O)c2ccc(OCCO)c(Cl)c2)cc1-c1nc2cccc(F)c2[nH]1] | 3-chloro-N-[5-(7-fluoro-1H-benzimidazol- 2-yl)-1-methyl-pyrazol-3-yl]-4-(2- hydroxyethoxy)benzamide | A-165 |
| 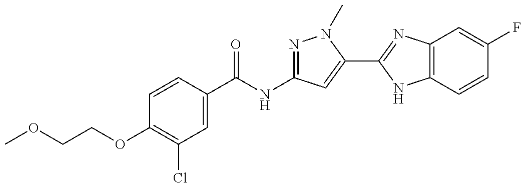 [SMILES: COCCOc1ccc(C(=O)Nc2cc(-c3nc4cc(F)ccc4[nH]3)n(C)n2)cc1Cl] | 3-chloro-N-[5-(5-fluoro-1H-benzimidazol- 2-yl)-1-methyl-pyrazol-3-yl]-4-(2- methoxyethoxy)benzamide | A-166 |
| 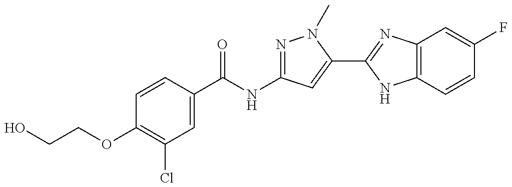 [SMILES: Cn1nc(NC(=O)c2ccc(OCCO)c(Cl)c2)cc1-c1nc2cc(F)ccc2[nH]1] | 3-chloro-N-[5-(5-fluoro-1H-benzimidazol- 2-yl)-1-methyl-pyrazol-3-yl]-4-(2- hydroxyethoxy)benzamide | A-167 |
| 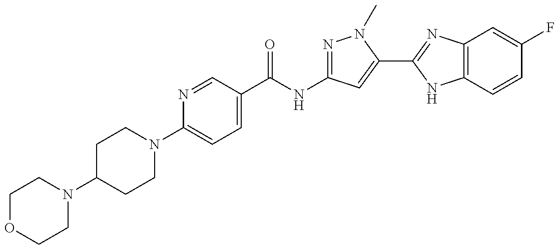 [SMILES: Cn1nc(NC(=O)c2ccc(N3CCC(N4CCOCC4)CC3)nc2)cc1-c1nc2cc(F)ccc2[nH]1] | N-[5-(5-fluoro-1H-benzimidazol-2-yl)-1- methyl-pyrazol-3-yl]-6-(4-morpholino-1- piperidyl)pyridine-3-carboxamide | A-168 |
| 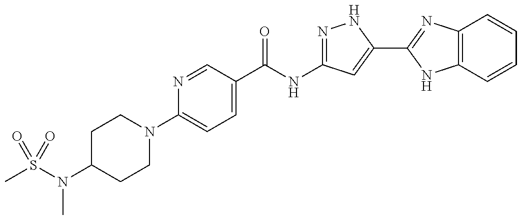 [SMILES: CN(C1CCN(c2ccc(C(=O)Nc3cc(-c4nc5ccccc5[nH]4)[nH]n3)cn2)CC1)S(C)(=O)=O] | N-[5-(1H-benzimidazol-2-yl)-1H-pyrazol-3- yl]-6-[4-[methyl(methylsulfonyl)amino]-1- piperidyl]pyridine-3-carboxamide | A-169 |
| 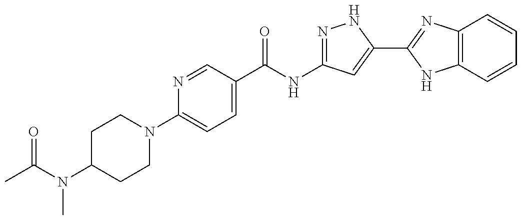 [SMILES: CC(=O)N(C)C1CCN(c2ccc(C(=O)Nc3cc(-c4nc5ccccc5[nH]4)[nH]n3)cn2)CC1] | 6-[4-[acetyl(methyl)amino]-1-piperidyl]-N- [5-(1H-benzimidazol-2-yl)-1H-pyrazol-3- yl]pyridine-3-carboxamide | A-170 |
| 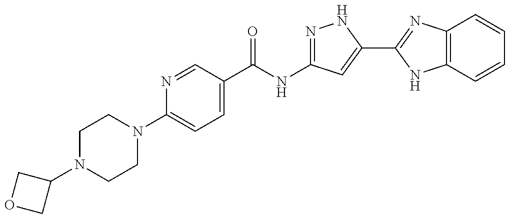 [SMILES: O=C(Nc1cc(-c2nc3ccccc3[nH]2)[nH]n1)c1ccc(N2CCN(C3COC3)CC2)nc1] | N-[5-(1H-benzimidazol-2-yl)-1H-pyrazol-3- yl]-6-[4-(oxetan-3-yl)piperazin-1- yl]pyridine-3-carboxamide | A-171 |
| 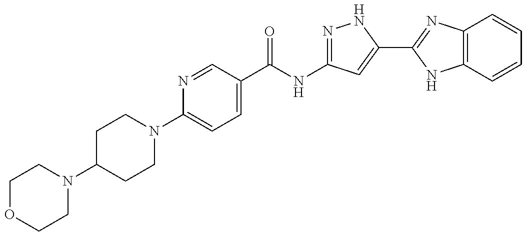 [SMILES: O=C(Nc1cc(-c2nc3ccccc3[nH]2)[nH]n1)c1ccc(N2CCC(N3CCOCC3)CC2)nc1] | N-[5-(1H-benzimidazol-2-yl)-1H-pyrazol-3- yl]-6-(4-morpholino-1-piperidyl)pyridine-3- carboxamide | A-172 |
| 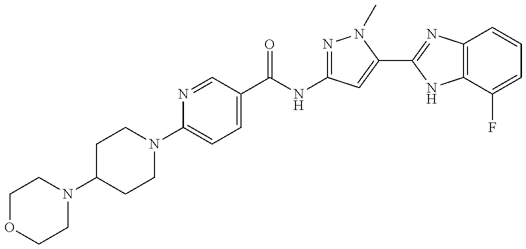 [SMILES: Cn1nc(NC(=O)c2ccc(N3CCC(N4CCOCC4)CC3)nc2)cc1-c1nc2cccc(F)c2[nH]1] | N-[5-(7-fluoro-1H-benzimidazol-2-yl)-1- methyl-pyrazol-3-yl]-6-(4-morpholino-1- piperidyl)pyridine-3-carboxamide | A-173 |
| 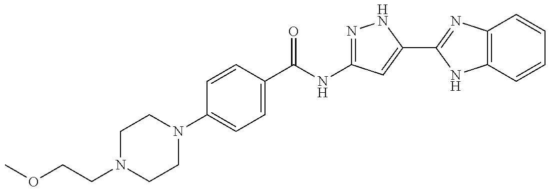 [SMILES: COCCN1CCN(c2ccc(C(=O)Nc3cc(-c4nc5ccccc5[nH]4)[nH]n3)cc2)CC1] | N-[5-(1H-benzimidazol-2-yl)-1H-pyrazol-3- yl]-4-[4-(2-methoxyethyl)piperazin-1- yl]benzamide | A-174 |
| 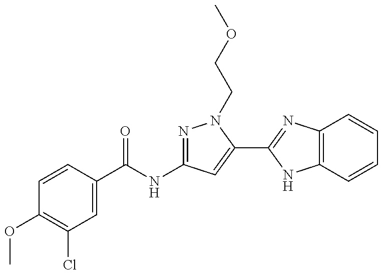 [SMILES: COCCn1nc(NC(=O)c2ccc(OC)c(Cl)c2)cc1-c1nc2ccccc2[nH]1] | N-[5-(1H-benzimidazol-2-yl)-1-(2- methoxyethyl)pyrazol-3-yl]-3-chloro-4- methoxy-benzamide | A-175 |
| 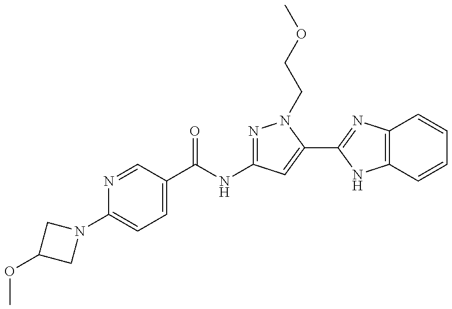 [SMILES: COCCn1nc(NC(=O)c2ccc(N3CC(OC)C3)nc2)cc1-c1nc2ccccc2[nH]1] | N-[5-(1H-benzimidazol-2-yl)-1-(2- methoxyethyl)pyrazol-3-yl]-6-(3- methoxyazetidin-1-yl)pyridine-3- carboxamide | A-176 |
| 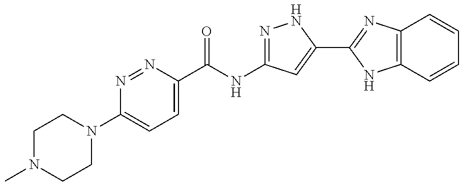 [SMILES: CN1CCN(c2ccc(C(=O)Nc3cc(-c4nc5ccccc5[nH]4)[nH]n3)nn2)CC1] | N-[5-(1H-benzimidazol-2-yl)-1H-pyrazol-3- yl]-6-(4-methylpiperazin-1-yl)pyridazine-3- carboxamide | A-177 |
| 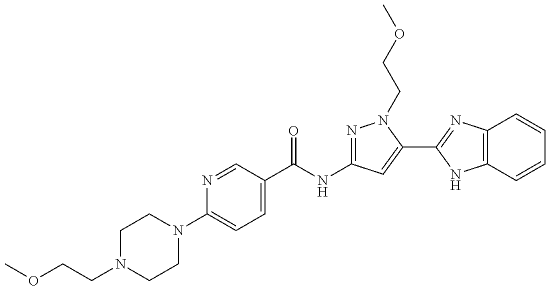 [SMILES: COCCN1CCN(c2ccc(C(=O)Nc3cc(-c4nc5ccccc5[nH]4)n(CCOC)n3)cn2)CC1] | N-[5-(1H-benzimidazol-2-yl)-1-(2- methoxyethyl)pyrazol-3-yl]-6-[4-(2- methoxyethyl)piperazin-1-yl]pyridine-3- carboxamide | A-178 |
| 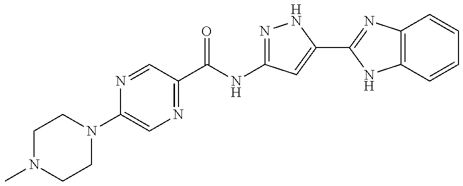 [SMILES: CN1CCN(c2cnc(C(=O)Nc3cc(-c4nc5ccccc5[nH]4)[nH]n3)cn2)CC1] | N-[5-(1H-benzimidazol-2-yl)-1H-pyrazol-3- yl]-5-(4-methylpiperazin-1-yl)pyrazine-2- carboxamide | A-179 |
| 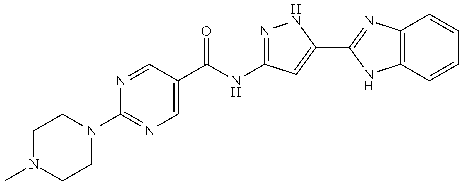 [SMILES: CN1CCN(c2ncc(C(=O)Nc3cc(-c4nc5ccccc5[nH]4)[nH]n3)cn2)CC1] | N-[5-(1H-benzimidazol-2-yl)-1H-pyrazol-3- yl]-2-(4-methylpiperazin-1-yl)pyrimidine- 5-carboxamide | A-180 |
| 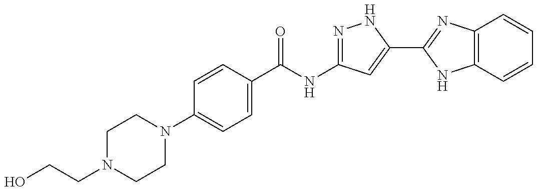 [SMILES: O=C(Nc1cc(-c2nc3ccccc3[nH]2)[nH]n1)c1ccc(N2CCN(CCO)CC2)cc1] | N-(5-(1H-benzoimidazol-2-yl)-1H-pyrazol- 3-yl)-4-(4-(2-hydroxyethyl)piperazin-1- yl)benzamide | A-181 |
| 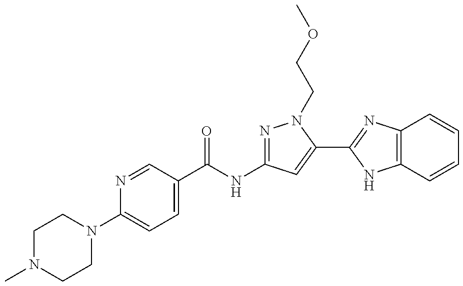 [SMILES: COCCn1nc(NC(=O)c2ccc(N3CCN(C)CC3)nc2)cc1-c1nc2ccccc2[nH]1] | N-[5-(1H-benzimidazol-2-yl)-1-(2- methoxyethyl)pyrazol-3-yl]-6-(4- methylpiperazin-1-yl)pyridine-3- carboxamide | A-182 |
| 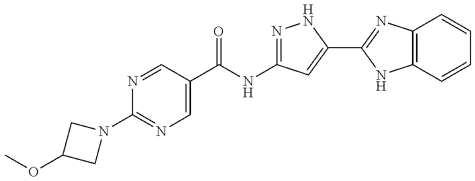 [SMILES: COC1CN(c2ncc(C(=O)Nc3cc(-c4nc5ccccc5[nH]4)[nH]n3)cn2)C1] | N-[5-(1H-benzimidazol-2-yl)-1H-pyrazol-3- yl]-2-(3-methoxyazetidin-1-yl)pyrimidine- 5-carboxamide | A-183 |
| 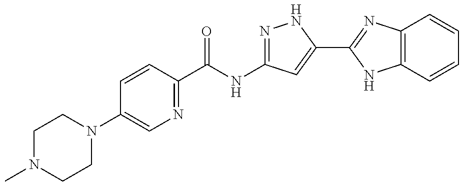 [SMILES: CN1CCN(c2ccc(C(=O)Nc3cc(-c4nc5ccccc5[nH]4)[nH]n3)nc2)CC1] | N-[5-(1H-benzimidazol-2-yl)-1H-pyrazol-3- yl]-5-(4-methylpiperazin-1-yl)pyridine-2- carboxamide | A-184 |
| 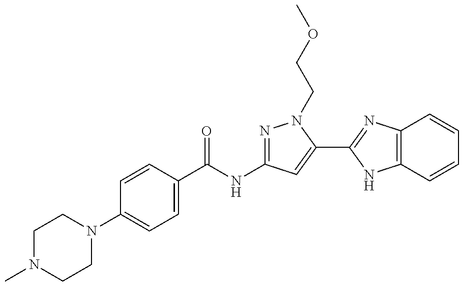 [SMILES: COCCn1nc(NC(=O)c2ccc(N3CCN(C)CC3)cc2)cc1-c1nc2ccccc2[nH]1] | N-[5-(1H-benzimidazol-2-yl)-1-(2- methoxyethyl)pyrazol-3-yl]-4-(4- methylpiperazin-1-yl)benzamide | A-185 |
| 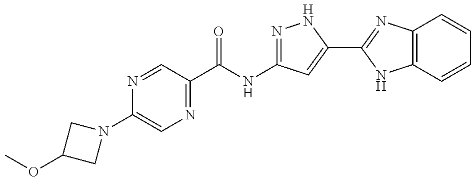 [SMILES: COC1CN(c2cnc(C(=O)Nc3cc(-c4nc5ccccc5[nH]4)[nH]n3)cn2)C1] | N-[5-(1H-benzimidazol-2-yl)-1H-pyrazol-3- yl]-5-(3-methoxyazetidin-1-yl)pyrazine-2- carboxamide | A-186 |
| 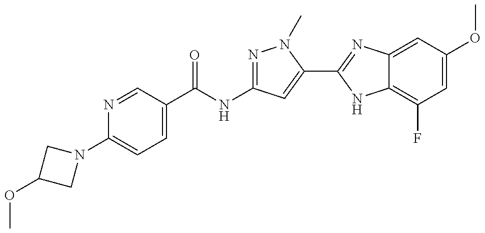 [SMILES: COc1cc(F)c2[nH]c(-c3cc(NC(=O)c4ccc(N5CC(OC)C5)nc4)nn3C)nc2c1] | N-[5-(7-fluoro-5-methoxy-1H-benzimi- dazol-2-yl)-1-methyl-pyrazol-3-yl]-6-(3- methoxyazetidin-1-yl)pyridine-3- carboxamide | A-187 |
| 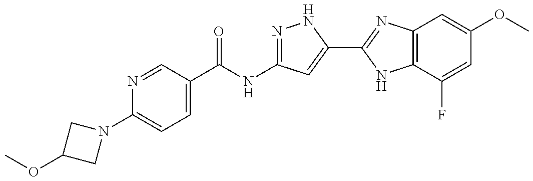 | N-[5-(7-fluoro-5-methoxy-1H- benzimidazol-2-yl)-1H-pyrazol-3-yl]-6- (3-methoxyazetidin- 1-yl)pyridine-3-carobxamide | A-188 |
| 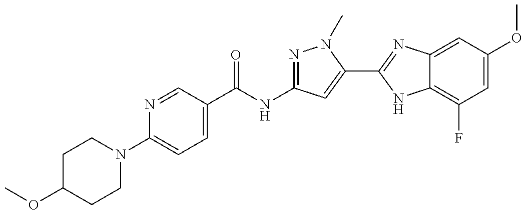 | N-[5-(7-fluoro-5-methoxy-1H- benzimidazol-2-yl)-1-methyl- pyrazol-3-yl]-6-(4-methozy- 1-piperidyl)pyridine-3-carboxamide | A-189 |
| 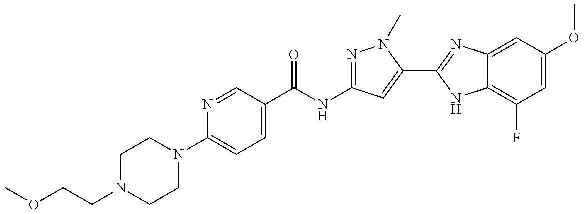 | N-[5-(7-fluoro-5-methoxy-1H- benzimidazol-2-yl)-1-methyl- pyrazol-3-yl]-6-[4-(2- methoxyethyl)piperazin-1-yl]pyridine-3- carboxamide | A-190 |
| 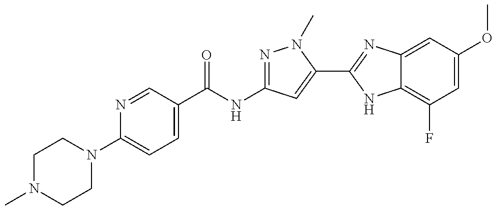 | N-[5-(7-fluoro-5-methoxy-1H- benzimidazol-2-yl)-1-methyl- pyrazol-3-yl]-6-(4- methylpiperazin-1-yl)pyridine-3- carboxamide | A-191 |
| 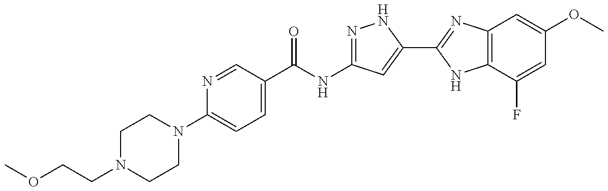 | N-[5-(7-fluoro-5-methoxy-1H- benzimidazol-2-yl)-1H-pyrazol- 3-yl]-6-[4-(2- methoxyethyl)piperazin-1-yl]pyridine-3- carboxamide | A-192 |
| 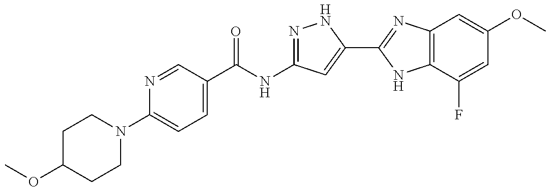 | N-[5-(7-fluroo-5-methoxy-1H- benzimidazol-2-yl)-1H- pyrazol-3-yl]-6-(4-methoxy-1- piperidyl)pyridine-3-carboxamide | A-193 |
| 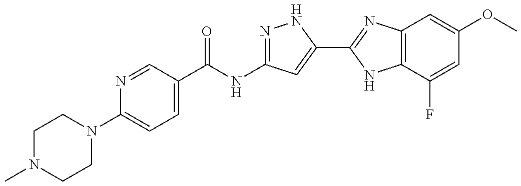 | N-[5-(7-fluoro-5-methoxy-1H-benzimi- dazol-2-yl)-1H-pyrazol-3-yl]-6-(4-methyl- piperazin-1-yl)pyridine-3-carboxamide | A-194 |
| 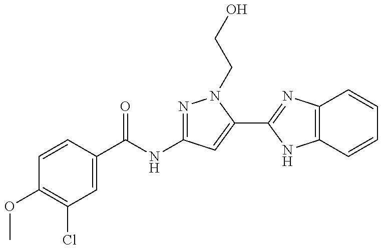 [SMILES: COc1ccc(C(=O)Nc2cc(-c3nc4ccccc4[nH]3)n(CCO)n2)cc1Cl] | N-[5-(1H-benzimidazol-2-yl)-1-(2- hydroxyethyl)pyrazol-3-yl]-3-chloro-4- methoxy-benzamide | A-195 |
| 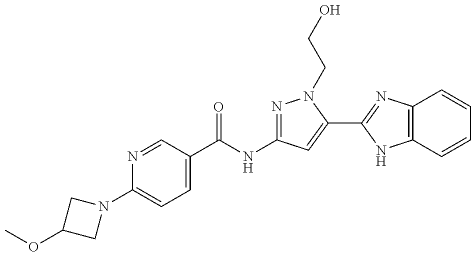 [SMILES: COC1CN(c2ccc(C(=O)Nc3cc(-c4nc5ccccc5[nH]4)n(CCO)n3)cn2)C1] | N-[5-(1H-benzimidaozl-2-yl)-1-(2- hydroxyethyl)pyrazol-3-yl]-6-(3- methoxyazetidin-1-yl)pyridine-3- carboxamide | A-196 |
| 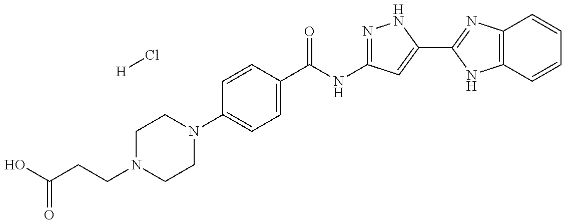 [SMILES: O=C(O)CCN1CCN(c2ccc(C(=O)Nc3cc(-c4nc5ccccc5[nH]4)[nH]n3)cc2)CC1 ; Cl] | 3-[4-[4-[[5-(1H-benzimidazol-2-yl)-1H- pyrazol-3-yl]carbamoyl]phenyl]piperazin-1- yl]propanoic acid hydrochloride | A-197 |
| 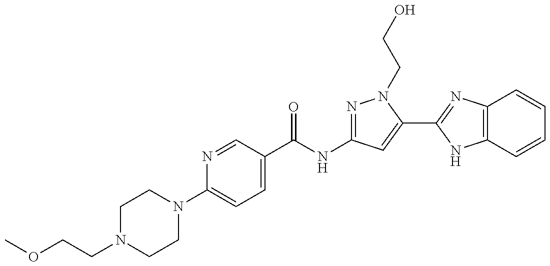 [SMILES: COCCN1CCN(c2ccc(C(=O)Nc3cc(-c4nc5ccccc5[nH]4)n(CCO)n3)cn2)CC1] | N-[5-(1H-benzimidazol-2-yl)-1-(2- hydroxyethyl)pyrazol-3-yl]-6-[4-(2- methoxyethyl)piperazin-1-yl]pyridine-3- carboxamide | A-198 |
| 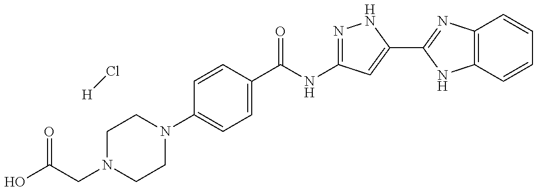 | 2-[4-[4-[[5-(1H-benzimidazol-2-yl)-1H- pyrazol-3-yl]carbamoyl]phenyl]piperazin-1- yl]acetic acid hydrochloride | A-199 |
| 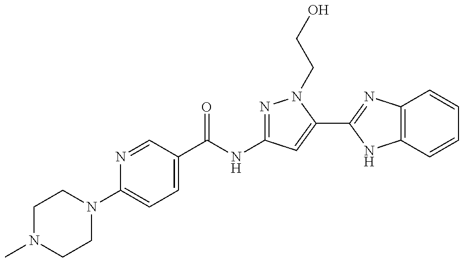 [SMILES: CN1CCN(c2ccc(C(=O)Nc3cc(-c4nc5ccccc5[nH]4)n(CCO)n3)cn2)CC1] | N-[5-(1H-benzimidazol-2-yl)-1-(2- hydroxyethyl)pyrazol-3-yl]-6-(4- methylpiperazin-1-yl)pyridine-3- carboxamide | A-200 |
| 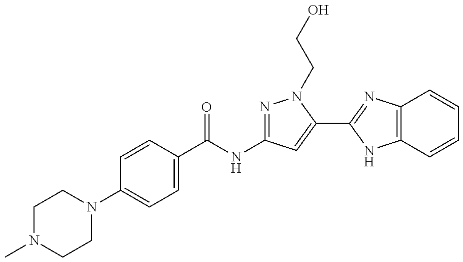 [SMILES: CN1CCN(c2ccc(C(=O)Nc3cc(-c4nc5ccccc5[nH]4)n(CCO)n3)cc2)CC1] | N-[5-(1H-benzimidazol-2-yl)-1-(2- hydroxyethyl)pyrazol-3-yl]-4-(4- methylpiperazin-1-yl)benzamide | A-201 |
| 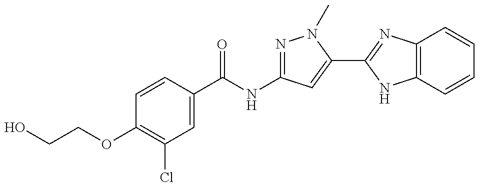 [SMILES: Cn1nc(NC(=O)c2ccc(OCCO)c(Cl)c2)cc1-c1nc2ccccc2[nH]1] | N-[5-(1H-benzimidazol-2-yl)-1-methyl- pyrazol-3-yl]-3-chloro-4-(2- hydroxyethoxy)benzamide | A-202 |
| 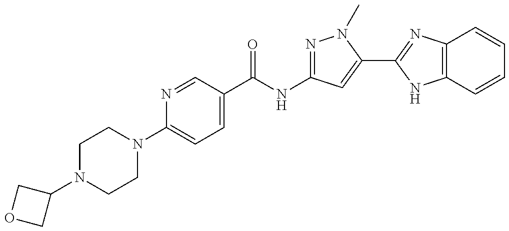 [SMILES: Cn1nc(NC(=O)c2ccc(N3CCN(C4COC4)CC3)nc2)cc1-c1nc2ccccc2[nH]1] | N-[5-(1H-benzimidazol-2-yl)-1-methyl- pyrazol-3-yl]-6-[4-(oxetan-3-yl)piperazin-1- yl]pyridine-3-carboxamide | A-203 |
| 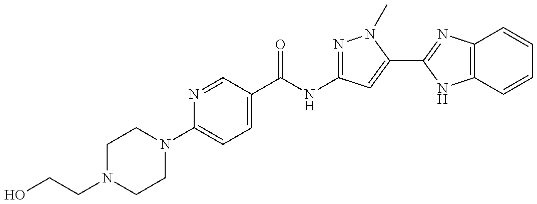 [SMILES: Cn1nc(NC(=O)c2ccc(N3CCN(CCO)CC3)nc2)cc1-c1nc2ccccc2[nH]1] | N-[5-(1H-benzimidazol-2-yl)-1-methyl- pyrazol-3-yl]-6-[4-(2-hydroxy- ethyl)piperazin-1-yl]pyridine-3- carboxamide | A-204 |
| 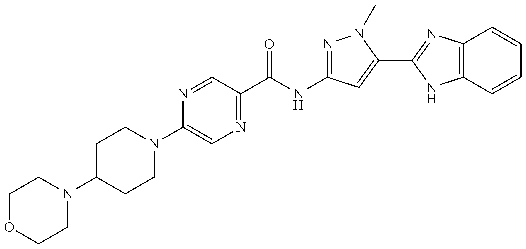 [SMILES: Cn1nc(NC(=O)c2cnc(N3CCC(N4CCOCC4)CC3)cn2)cc1-c1nc2ccccc2[nH]1] | N-[5-(1H-benzimidazol-2-yl)-1-methyl- pyrazol-3-yl]-5-(4-morpholino-1- piperidyl)pyrazine-2-carboxamide | A-205 |
| 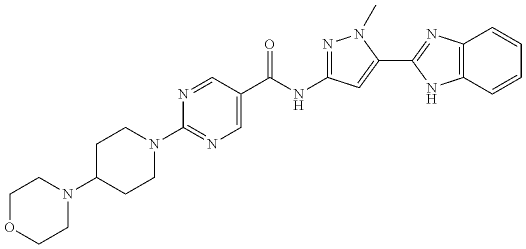 [SMILES: Cn1nc(NC(=O)c2cnc(N3CCC(N4CCOCC4)CC3)nc2)cc1-c1nc2ccccc2[nH]1] | N-[5-(1H-benzimidazol-2-yl)-1-methyl- pyrazol-3-yl]-2-(4-morpholino-1- piperidyl)pyrimidine-5-carboxamide | A-206 |
| 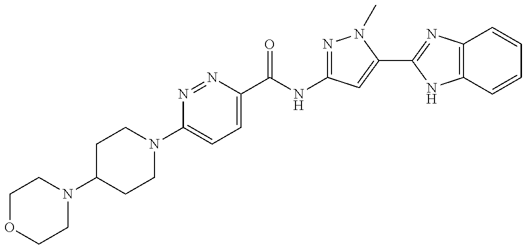 [SMILES: Cn1nc(NC(=O)c2ccc(N3CCC(N4CCOCC4)CC3)nn2)cc1-c1nc2ccccc2[nH]1] | N-[5-(1H-benzimidazol-2-yl)-1-methyl- pyrazol-3-yl]-6-(4-morpholino-1- piperidyl)pyridazine-3-carboxamide | A-207 |
| 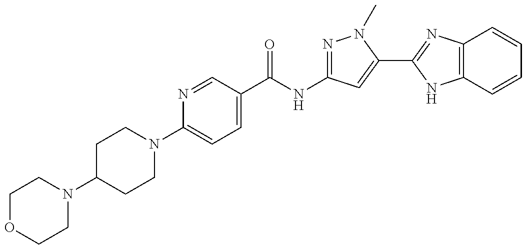 [SMILES: Cn1nc(NC(=O)c2ccc(N3CCC(N4CCOCC4)CC3)nc2)cc1-c1nc2ccccc2[nH]1] | N-[5-(1H-benzimidazol-2-yl)-1-methyl- pyrazol-3-yl]-6-(4-morpholino-1- piperidyl)pyridine-3-carboxamide | A-208 |
| 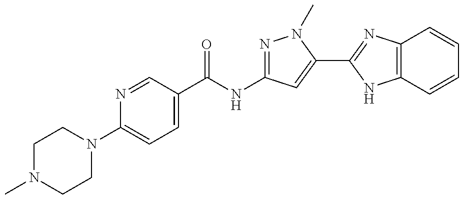 [SMILES: CN1CCN(c2ccc(C(=O)Nc3cc(-c4nc5ccccc5[nH]4)n(C)n3)cn2)CC1] | N-[5-(1H-benzimidazol-2-yl)-1-methyl- pyrazol-3-yl]-6-(4-methylpiperazin-1- yl)pyridine-3-carboxamide | A-209 |
| 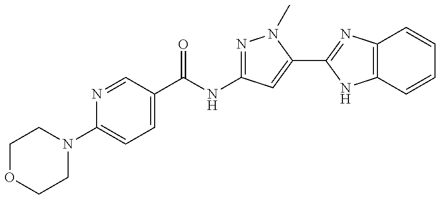 [SMILES: Cn1nc(NC(=O)c2ccc(N3CCOCC3)nc2)cc1-c1nc2ccccc2[nH]1] | N-[5-(1H-benzimidazol-2-yl)-1-methyl- pyrazol-3-yl]-6-morpholino-pyridine-3- carboxamide | A-210 |
| 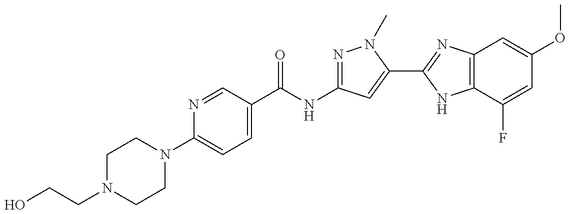 [SMILES: COc1cc(F)c2[nH]c(-c3cc(NC(=O)c4ccc(N5CCN(CCO)CC5)nc4)nn3C)nc2c1] | N-[5-(7-fluoro-5-methoxy-1H- benzimidazol-2-yl)-1-methyl- pyrazol-3-yl]-6-[4-(2- hydroxyethyl)piperazin-1-yl]pyridine-3- carboxamide | A-211 |
| 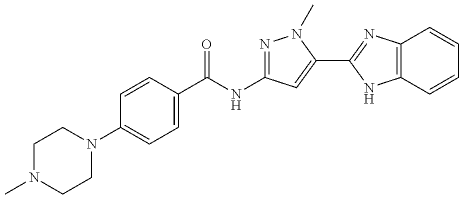 [SMILES: CN1CCN(c2ccc(C(=O)Nc3cc(-c4nc5ccccc5[nH]4)n(C)n3)cc2)CC1] | N-[5-(1H-benzimidazol-2-yl)-1-methyl- pyrazol-3-yl]-4-(4-methylpiperazin-1- yl)benzamide | A-212 |
| 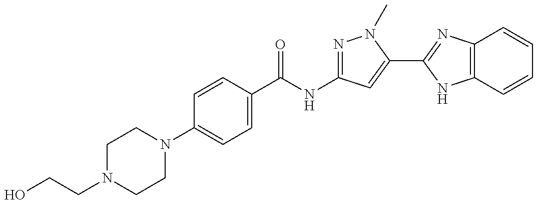 [SMILES: Cn1nc(NC(=O)c2ccc(N3CCN(CCO)CC3)cc2)cc1-c1nc2ccccc2[nH]1] | N-[5-(1H-benzimidazol-2-yl)-1-methyl- pyrazol-3-yl]-4-[4-(2-hydroxyethyl)pipera- zin-1-yl]benzamide | A-213 |
| 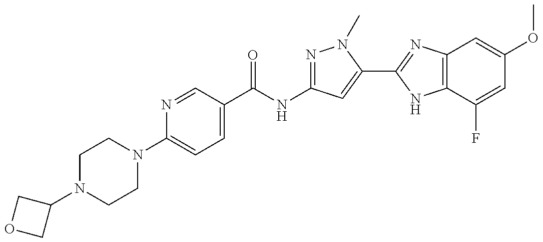 [SMILES: COc1cc(F)c2[nH]c(-c3cc(NC(=O)c4ccc(N5CCN(C6COC6)CC5)nc4)nn3C)nc2c1] | N-[5-(7-fluoro-5-methoxy-1H- benzimidazol-2-yl)-1-methyl-pyrazol- 3-yl]-6-[4-(oxetan-3- yl)piperazin-1-yl]pyridine-3-carboxamide | A-214 |
| 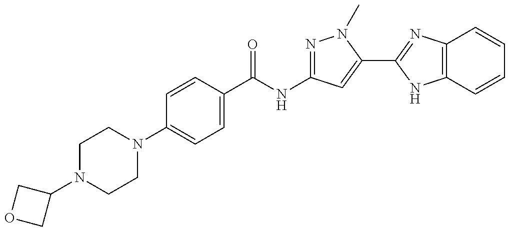 [SMILES: Cn1nc(NC(=O)c2ccc(N3CCN(C4COC4)CC3)cc2)cc1-c1nc2ccccc2[nH]1] | N-[5-(1H-benzimidazol-2-yl)-1-methyl- pyrazol-3-yl]-4-[4-(oxetan-3-yl)piperazin- 1-yl]benzamide | A-215 |
| 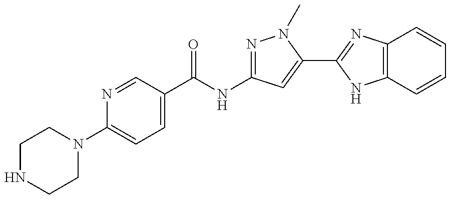 [SMILES: Cn1nc(NC(=O)c2ccc(N3CCNCC3)nc2)cc1-c1nc2ccccc2[nH]1] | N-[5-(1H-benzimidazol-2-yl)-1-methyl- pyrazol-3-yl]-6-piperazin-1-yl-pyridine-3- carboxamide | A-216 |
| 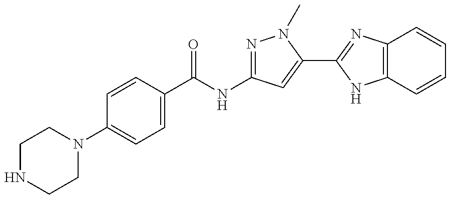 [SMILES: Cn1nc(NC(=O)c2ccc(N3CCNCC3)cc2)cc1-c1nc2ccccc2[nH]1] | N-[5-(1H-benzimidazol-2-yl)-1-methyl- pyrazol-3-yl]-4-piperazin-1-yl-benzamide | A-217 |
| 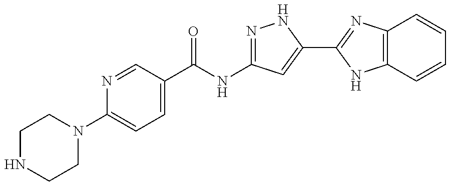 [SMILES: O=C(Nc1cc(-c2nc3ccccc3[nH]2)[nH]n1)c1ccc(N2CCNCC2)nc1] | N-[5-(1H-benzimidazol-2-yl)-1H-pyrazol- 3-yl]-6-piperazin-1- yl-pyridine-3-carboxamide | A-218 |
| 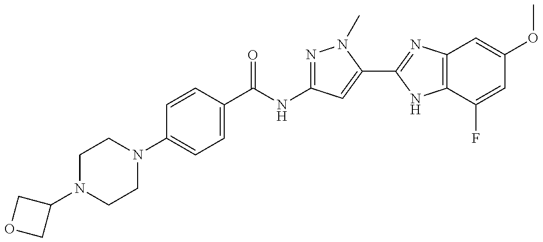 [SMILES: COc1cc(F)c2[nH]c(-c3cc(NC(=O)c4ccc(N5CCN(C6COC6)CC5)cc4)nn3C)nc2c1] | N-[5-(7-fluoro-5-methoxy-1H- benzimidazol-2-yl)-1-methyl- pyrazol-3-yl]-4-[4-(oxetan-3- yl)piperazin-1-yl]benzamide | A-219 |
| 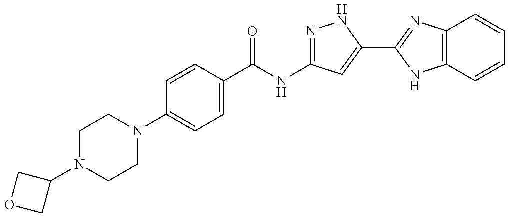 [SMILES: O=C(Nc1cc(-c2nc3ccccc3[nH]2)[nH]n1)c1ccc(N2CCN(C3COC3)CC2)cc1] | N-[5-(1H-benzimidazol-2-yl)-1H-pyrazol- 3-yl]-4-[4-(oxetan-3-yl)piperazin-1- yl]benzamide | A-220 |
| 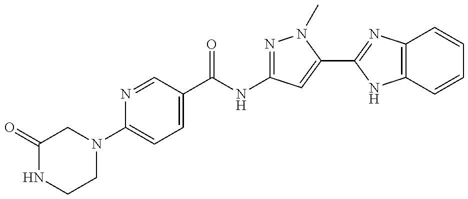 [SMILES: Cn1nc(NC(=O)c2ccc(N3CCNC(=O)C3)nc2)cc1-c1nc2ccccc2[nH]1] | N-[5-(1H-benzimidazol-2-yl)-1-methyl- pyrazol-3-yl]-6-(3-oxopiperazin-1- yl)pyridine-3-carboxamide | A-221 |
| 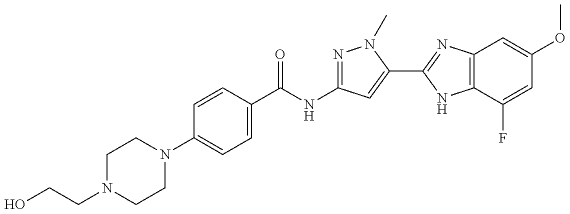 [SMILES: COc1cc(F)c2[nH]c(-c3cc(NC(=O)c4ccc(N5CCN(CCO)CC5)cc4)nn3C)nc2c1] | N-[5-(7-fluoro-5-methoxy-1H- benzimidazol- 2-yl)-1-methyl-pyrazol-3-yl]-4-[4-(2- hydroxyethyl)piperazin-1-yl]benzamide | A-222 |
| 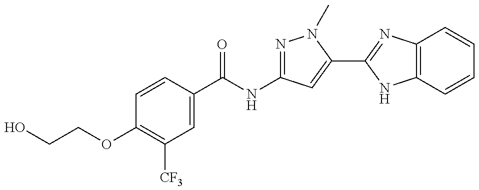 [SMILES: Cn1nc(NC(=O)c2ccc(OCCO)c(C(F)(F)F)c2)cc1-c1nc2ccccc2[nH]1] | N-[5-(1H-benzimidazol-2-yl)-1-methyl- pyrazol-3-yl]-4-(2-hydroxyethoxy)-3- (trifluoromethyl)benzamide | A-223 |
| 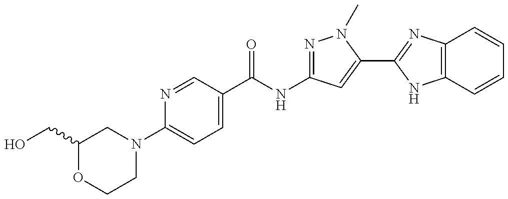 [SMILES: Cn1nc(NC(=O)c2ccc(N3CCOC(CO)C3)nc2)cc1-c1nc2ccccc2[nH]1] | N-[5-(1H-benzimidazol-2-yl)-1-methyl- pyrazol-3-yl]-6-[2- (hydroxymethyl)morpholin-4-yl]pyridine-3- carboxamide | A-224 |
| 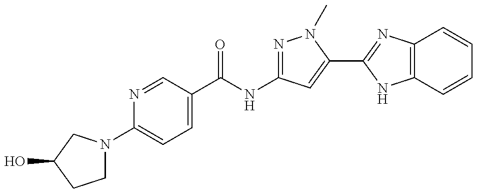 [SMILES: Cn1nc(NC(=O)c2ccc(N3CC[C@@H](O)C3)nc2)cc1-c1nc2ccccc2[nH]1] | N-[5-(1H-benzimidazol-2-yl)-1-methyl- pyrazol-3-yl]-6-[(3R)-3-hydroxypyrrolidin- 1-yl]pyridine-3-carboxamide | A-225 |
| 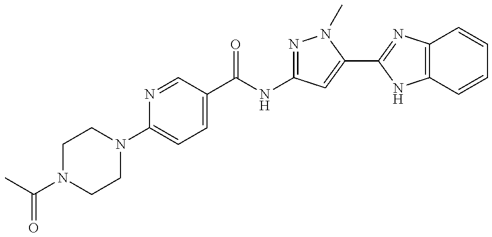 [SMILES: CC(=O)N1CCN(c2ccc(C(=O)Nc3cc(-c4nc5ccccc5[nH]4)n(C)n3)cn2)CC1] | 6-(4-acetylpiperazin-1-yl)-N-[5-(1H- benzimidazol-2-yl)-1-methyl-pyrazol-3- yl]pyridine-3-carboxamide | A-226 |
| 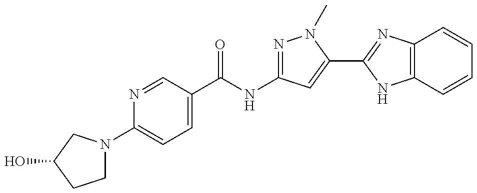 [SMILES: Cn1nc(NC(=O)c2ccc(N3CC[C@H](O)C3)nc2)cc1-c1nc2ccccc2[nH]1] | N-[5-(1H-benzimidazol-2-yl)-1-methyl- pyrazol-3-yl]-6-[(3S)-3-hydroxypyrrolidin- 1-yl]pyridine-3-carboxamide | A-227 |
| 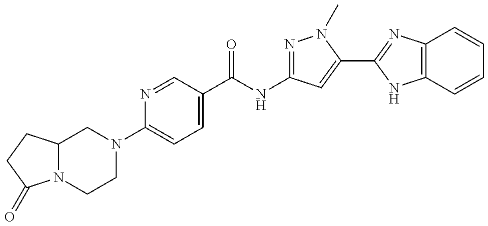 [SMILES: Cn1nc(NC(=O)c2ccc(N3CCN4C(=O)CCC4C3)nc2)cc1-c1nc2ccccc2[nH]1] | N-[5-(1H-benzimidazol-2-yl)-1-methyl- pyrazol-3-yl]-6-(6-oxo-1,3,4,7,8,8a- hexahydropyrrolo[1,2-a]pyrazin-2- yl)pyridine-3-carboxamide | A-228 |
| 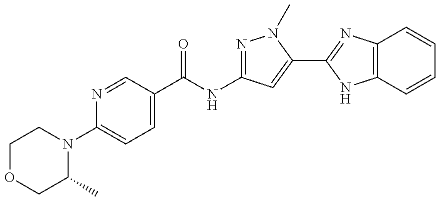 [SMILES: C[C@@H]1COCCN1c1ccc(C(=O)Nc2cc(-c3nc4ccccc4[nH]3)n(C)n2)cn1] | N-[5-(1H-benzimidazol-2-yl)-1-methyl- pyrazol-3-yl]-6-[(3R)-3-methylmorpholin-4- yl]pyridine-3-carboxamide | A-229 |
| 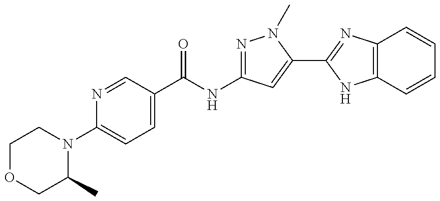 [SMILES: C[C@H]1COCCN1c1ccc(C(=O)Nc2cc(-c3nc4ccccc4[nH]3)n(C)n2)cn1] | N-[5-(1H-benzimidazol-2-yl)-1-methyl- pyrazol-3-yl]-6-[(3S)-3-methylmorpholin-4- yl]pyridine-3-carboxamide | A-230 |
| 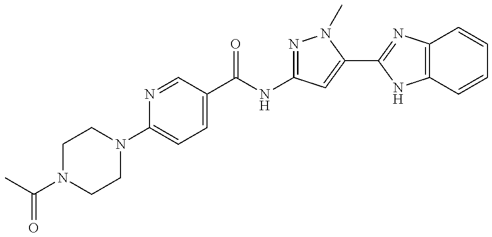 [SMILES: CC(=O)N1CCN(c2ccc(C(=O)Nc3cc(-c4nc5ccccc5[nH]4)n(C)n3)cn2)CC1] | 6-(4-acetylpiperazin-1-yl)-N-[5-(1H- benzimidazol-2-yl)-1-methyl-pyrazol-3- yl]pyridine-3-carboxamide | A-231 |
| 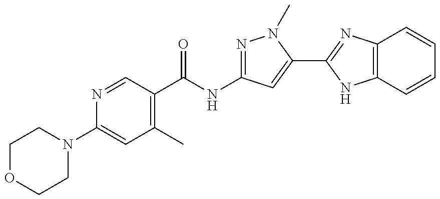 [SMILES: Cc1cc(N2CCOCC2)ncc1C(=O)Nc1cc(-c2nc3ccccc3[nH]2)n(C)n1] | N-[5-(1H-benzimidazol-2-yl)-1-methyl- pyrazol-3-yl]-4-methyl-6-morpholino- pyridine-3-carboxamide | A-232 |
| 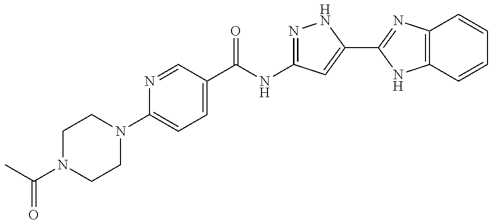 [SMILES: CC(=O)N1CCN(c2ccc(C(=O)Nc3cc(-c4nc5ccccc5[nH]4)[nH]n3)cn2)CC1] | 6-(4-acetylpiperazin-1-yl)-N-[5-(1H- benzimidazol-2-yl)-1-[(4- methoxyphenyl)methyl]pyrazol-3- yl]pyridine-3-carboxamide | A-233 |
| 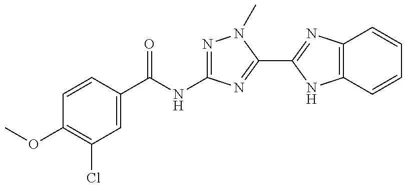 [SMILES: COc1ccc(C(=O)Nc2nc(-c3nc4ccccc4[nH]3)n(C)n2)cc1Cl] | N-[5-(1H-benzimidazol-2-yl)-1-methyl- 1,2,4-triazol-3-yl]-3-chloro-4-methoxy- benzamide | A-234 |
| 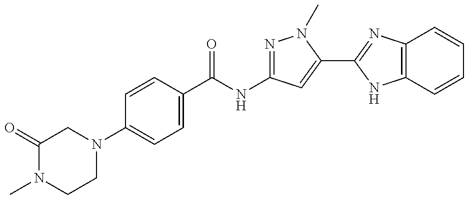 [SMILES: CN1CCN(c2ccc(C(=O)Nc3cc(-c4nc5ccccc5[nH]4)n(C)n3)cc2)CC1=O] | N-[5-(1H-benzimidazol-2-yl)-1-methyl- pyrazol-3-yl]-4-(4-methyl-3-oxo-piperazin- 1-yl)benzamide | A-235 |
| 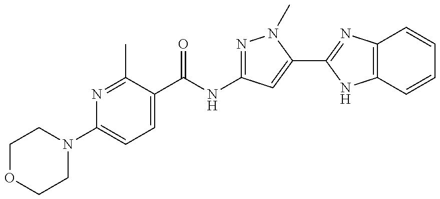 [SMILES: Cc1nc(N2CCOCC2)ccc1C(=O)Nc1cc(-c2nc3ccccc3[nH]2)n(C)n1] | N-[5-(1H-benzimidazol-2-yl)-1-methyl- pyrazol-3-yl]-2-methyl-6-morpholino- pyridine-3-carboxamide | A-236 |
| 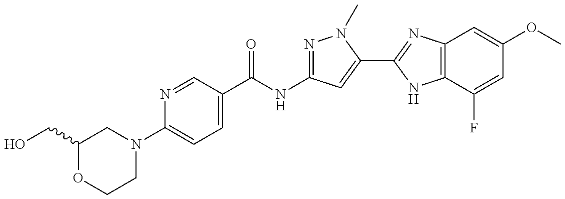 [SMILES: COc1cc(F)c2[nH]c(-c3cc(NC(=O)c4ccc(N5CCOC(CO)C5)nc4)nn3C)nc2c1] | N-[5-(7-fluoro-5-methoxy-1H- benzimidazol-2-yl)-1-methyl-pyrazol-3- yl]-6-[2-(hydroxymethyl)morpholin- 4-yl]pyridine-3-carboxamide | A-237 |
| 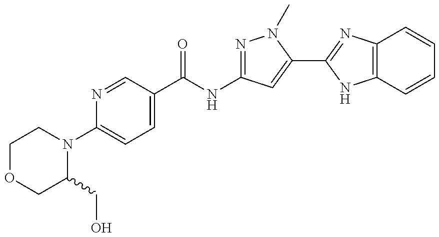 [SMILES: Cn1nc(NC(=O)c2ccc(N3CCOCC3CO)nc2)cc1-c1nc2ccccc2[nH]1] | N-[5-(1H-benzimidazol-2-yl)-1-methyl- pyrazol-3-yl]-6-[3- (hydroxymethyl)morpholin-4-yl]pyridine-3- carboxamide | A-238 |
| 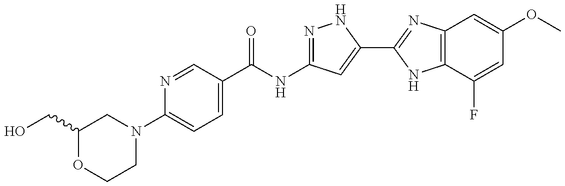 [SMILES: COc1cc(F)c2[nH]c(-c3cc(NC(=O)c4ccc(N5CCOC(CO)C5)nc4)n[nH]3)nc2c1] | N-[5-(7-fluoro-5-methoxy-1H-benzimi- dazol-2-yl)-1H-pyrazol-3-yl]-6-[2- (hydroxymethyl)morpholin-4-yl]pyridine-3- carboxamide | A-239 |
| 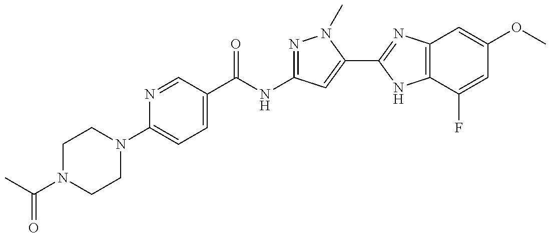 [SMILES: COc1cc(F)c2[nH]c(-c3cc(NC(=O)c4ccc(N5CCN(C(C)=O)CC5)nc4)nn3C)nc2c1] | 6-(4-acetylpiperazin-1-yl)-N-[5-(7-fluoro-5- methoxy-1H-benzimidazol-2-yl)-1-methyl- pyrazol-3-yl]pyridine-3-carboxamide | A-240 |
| 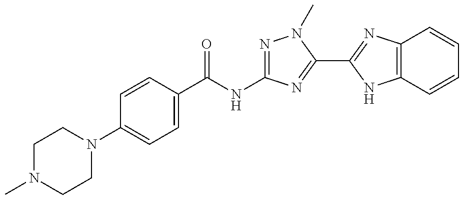 [SMILES: CN1CCN(c2ccc(C(=O)Nc3nc(-c4nc5ccccc5[nH]4)n(C)n3)cc2)CC1] | N-[5-(1H-benzimidazol-2-yl)-1-methyl- 1,2,4-triazol-3-yl]-4-(4-methylpiperazin-1- yl)benzamide | A-241 |
| 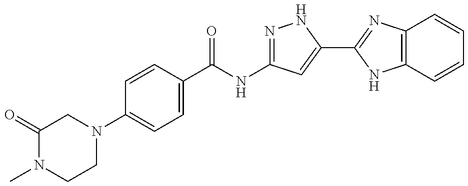 [SMILES: CN1CCN(c2ccc(C(=O)Nc3cc(-c4nc5ccccc5[nH]4)[nH]n3)cc2)CC1=O] | N-[5-(1H-benzimidazol-2-yl)-1H-pyrazol-3- yl]-4-(4-methyl-3-oxo-piperazin-1- yl)benzamide | A-242 |
| 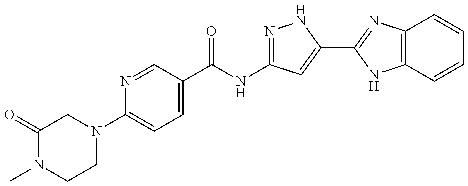 [SMILES: CN1CCN(c2ccc(C(=O)Nc3cc(-c4nc5ccccc5[nH]4)[nH]n3)cn2)CC1=O] | N-[5-(1H-benzimidazol-2-yl)-1H-pyrazol-3- yl]-6-(4-methyl-3-oxo-piperazin-1- yl)pyridine-3-carboxamide | A-243 |
| 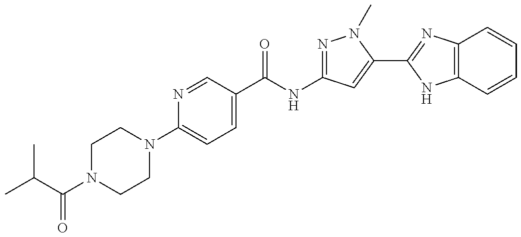 [SMILES: CC(C)C(=O)N1CCN(c2ccc(C(=O)Nc3cc(-c4nc5ccccc5[nH]4)n(C)n3)cn2)CC1] | N-[5-(1H-benzimidazol-2-yl)-1-methyl- pyrazol-3-yl]-6-[4-(2- methylpropanoyl)piperazin-1-yl]pyridine-3- carboxamide | A-244 |
| 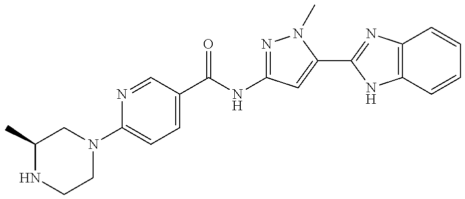 [SMILES: C[C@H]1CN(c2ccc(C(=O)Nc3cc(-c4nc5ccccc5[nH]4)n(C)n3)cn2)CCN1] | N-[5-(1H-benzimidazol-2-yl)-1-methyl- pyrazol-3-yl]-6-[(3S)-3-methylpiperazin-1- yl]pyridine-3-carboxamide | A-245 |
| 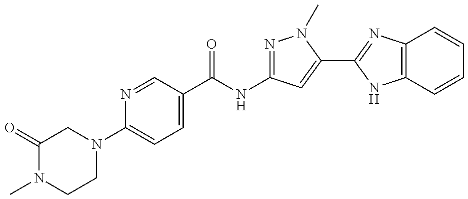 [SMILES: CN1CCN(c2ccc(C(=O)Nc3cc(-c4nc5ccccc5[nH]4)n(C)n3)cn2)CC1=O] | N-[5-(1H-benzimidazol-2-yl)-1-methyl- pyrazol-3-yl]-6-(4-methyl-3-oxo-piperazin- 1-yl)pyridine-3-carboxamide | A-246 |
| 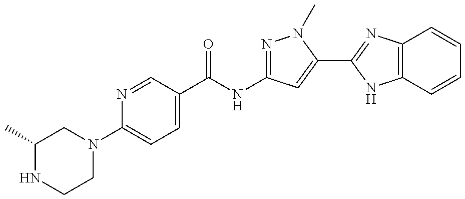 [SMILES: C[C@@H]1CN(c2ccc(C(=O)Nc3cc(-c4nc5ccccc5[nH]4)n(C)n3)cn2)CCN1] | N-[5-(1H-benzimidazol-2-yl)-1-methyl- pyrazol-3-yl]-6-[(3R)-3-methylpiperazin-1- yl]pyridine-3-carboxamide | A-247 |
| 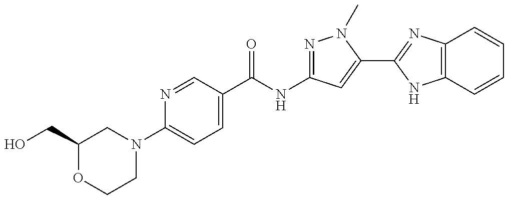 [SMILES: Cn1nc(NC(=O)c2ccc(N3CCO[C@@H](CO)C3)nc2)cc1-c1nc2ccccc2[nH]1] | N-[5-(1H-benzimidazol-2-yl)-1-methyl- pyrazol-3-yl]-6-[(2R)-2- (hydroxymethyl)morpholin-4-yl]pyridine-3- carboxamide | A-248 |
| 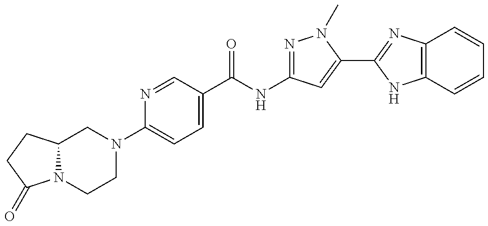 [SMILES: Cn1nc(NC(=O)c2ccc(N3CCN4C(=O)CC[C@@H]4C3)nc2)cc1-c1nc2ccccc2[nH]1] | 6-[(8aR)-6-oxo-1,3,4,7,8,8a- hexahydropyrrolo[1,2-a]pyrazin-2-yl]-N-[5- (1H-benzimidazol-2-yl)-1-methyl-pyrazol- 3-yl]pyridine-3-carboxamide | A-249 |
| 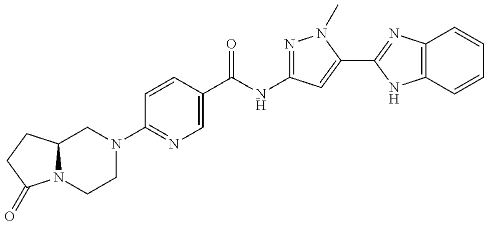 [SMILES: Cn1nc(NC(=O)c2ccc(N3CCN4C(=O)CC[C@H]4C3)nc2)cc1-c1nc2ccccc2[nH]1] | 6-[(8aS)-6-oxo-1,3,4,7,8,8a- hexahydropyrrolo[1,2-a]pyrazin-2-yl]-N-[5- (1H-benzimidazol-2-yl)-1-methyl-pyrazol- 3-yl]pyridine-3-carboxamide | A-250 |
| 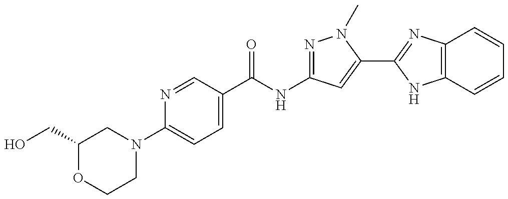 [SMILES: Cn1nc(NC(=O)c2ccc(N3CCO[C@H](CO)C3)nc2)cc1-c1nc2ccccc2[nH]1] | N-[5-(1H-benzimidazol-2-yl)-1-methyl- pyrazol-3-yl]-6-[2S)-2- (hydroxymethyl)morpholin-4-yl]pyridine-3- carboxamide | A-251 |
| 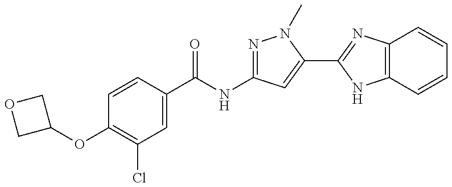 [SMILES: Cn1nc(NC(=O)c2ccc(OC3COC3)c(Cl)c2)cc1-c1nc2ccccc2[nH]1] | N-[5-(1H-benzimidazol-2-yl)-1-methyl- pyrazol-3-yl]-3-chloro-4-(oxetan-3- yloxy)benzamide | A-252 |
| 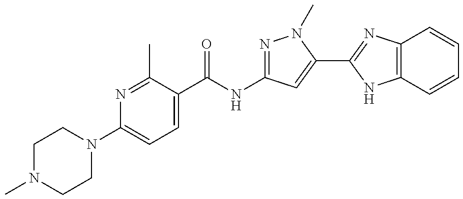 [SMILES: Cc1nc(N2CCN(C)CC2)ccc1C(=O)Nc1cc(-c2nc3ccccc3[nH]2)n(C)n1] | N-[5-(1H-benzimidazol-2-yl)-1-methyl- pyrazol-3-yl]-2-methyl-6-(4-methyl- piperazin-1-yl)pyridine-3-carboxamide | A-253 |
| 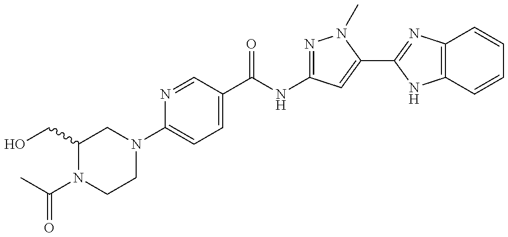 [SMILES: CC(=O)N1CCN(c2ccc(C(=O)Nc3cc(-c4nc5ccccc5[nH]4)n(C)n3)cn2)CC1CO] | 6-[4-acetyl-3-(hydroxymethyl)piperazin-1- yl]-N-[5-(1H-benzimidazol-2-yl)-1-methyl- pyrazol-3-yl]pyridine-3-carboxamide | A-254 |
| 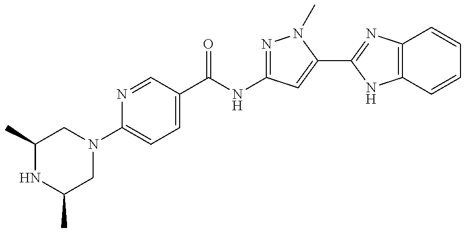 [SMILES: C[C@@H]1CN(c2ccc(C(=O)Nc3cc(-c4nc5ccccc5[nH]4)n(C)n3)cn2)C[C@H](C)N1] | N-[5-(1H-benzimidazol-2-yl)-1-methyl- pyrazol-3-yl]-6-[(3R,5S)-3,5- dimethylpiperazin-1-yl]pyridine-3- carboxamide | A-255 |
| 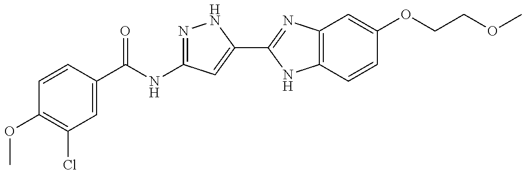 [SMILES: COCCOc1ccc2[nH]c(-c3cc(NC(=O)c4ccc(OC)c(Cl)c4)n[nH]3)nc2c1] | 3-chloro-4-methoxy-N-[5-[5-(2- methoxyethoxy)-1H-benzimidazol-2-yl]- 1H-pyrazol-3-yl]benzamide | A-256 |
| 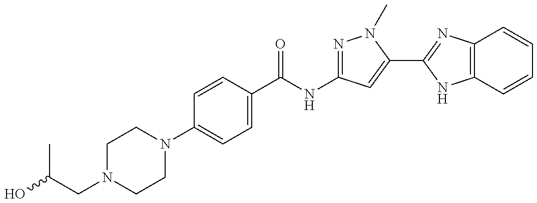 [SMILES: CC(O)CN1CCN(c2ccc(C(=O)Nc3cc(-c4nc5ccccc5[nH]4)n(C)n3)cc2)CC1] | N-[5-(1H-benzimidazol-2-yl)-1-methyl- pyrazol-3-yl]-4-[4-(2- hydroxypropyl)piperazin-1-yl]benzamide | A-257 |
| 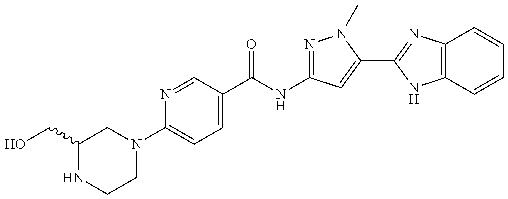 [SMILES: Cn1nc(NC(=O)c2ccc(N3CCNC(CO)C3)nc2)cc1-c1nc2ccccc2[nH]1] | N-[5-(1H-benzimidazol-2-yl)-1-methyl- pyrazol-3-yl]-6-[3-(hydroxymethyl)pipera- zin-1-yl]pyridine-3-carboxamide | A-258 |
| 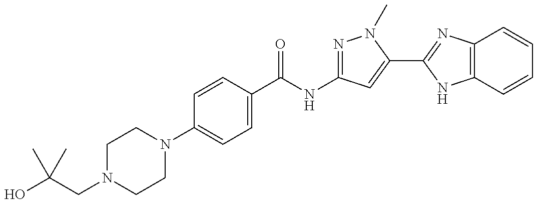 [SMILES: Cn1nc(NC(=O)c2ccc(N3CCN(CC(C)(C)O)CC3)cc2)cc1-c1nc2ccccc2[nH]1] | N-[5-(1H-benzimidazol-2-yl)-1-methyl- pyrazol-3-yl]-4-[4-(2-hydroxy-2-methyl- propyl)piperazin-1-yl]benzamide | A-259 |
| 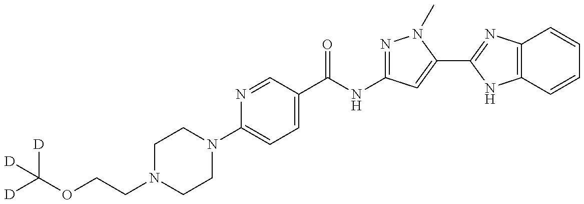 [SMILES: [2H]C([2H])([2H])OCCN1CCN(c2ccc(C(=O)Nc3cc(-c4nc5ccccc5[nH]4)n(C)n3)cn2)CC1] | N-[5-(1H-benzimidazol-2-yl)-1-methyl- pyrazol-3-yl]-6-[4-[2- (trideuteriomethoxy)ethyl]piperazin-1- yl]pyridine-3-carboxamide | A-260 |
| 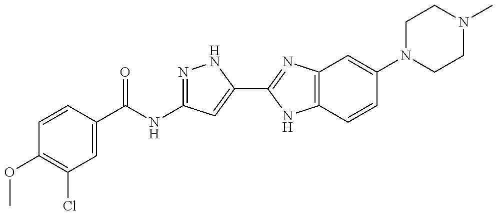 [SMILES: COc1ccc(C(=O)Nc2cc(-c3nc4cc(N5CCN(C)CC5)ccc4[nH]3)[nH]n2)cc1Cl] | 3-chloro-4-methoxy-N-[5-[5-(4- methylpiperazin-1-yl)-1H-benzimidazol-2- yl]-1H-pyrazol-3-yl]benzamide | A-261 |
| 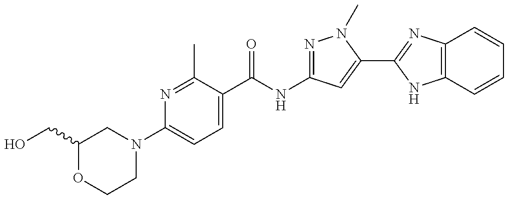 [SMILES: Cc1nc(N2CCOC(CO)C2)ccc1C(=O)Nc1cc(-c2nc3ccccc3[nH]2)n(C)n1] | N-[5-(1H-benzimidazol-2-yl)-1-methyl- pyrazol-3-yl]-6-[2- (hydroxymethyl)morpholin-4-yl]-2-methyl- pyridine-3-carboxamide | A-262 |
| 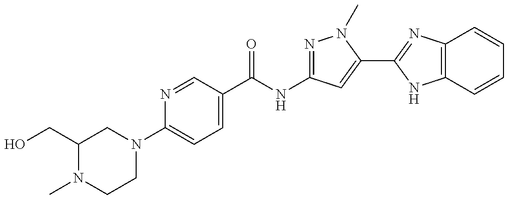 [SMILES: CN1CCN(c2ccc(C(=O)Nc3cc(-c4nc5ccccc5[nH]4)n(C)n3)cn2)CC1CO] | N-[5-(1H-benzimidazol-2-yl)-1-methyl- pyrazol-3-yl]-6-[3-(hydroxymethyl)-4- methyl-piperazin-1-yl]pyridine-3- carboxamide | A-263 |
| 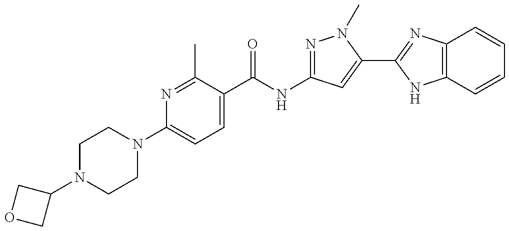 [SMILES: Cc1nc(N2CCN(C3COC3)CC2)ccc1C(=O)Nc1cc(-c2nc3ccccc3[nH]2)n(C)n1] | N-[5-(1H-benzimidazol-2-yl)-1-methyl- pyrazol-3-yl]-2-methyl-6-[4-(oxetan-3- yl)piperazin-1-yl]pyridine-3-carboxamide | A-264 |
| 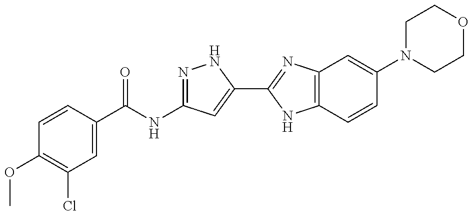 [SMILES: COc1ccc(C(=O)Nc2cc(-c3nc4cc(N5CCOCC5)ccc4[nH]3)[nH]n2)cc1Cl] | 3-chloro-4-methoxy-N-[5-(5-morpholino- 1H-benzimidazol-2-yl)-1H-pyrazol-3- yl]benzamide | A-265 |
| 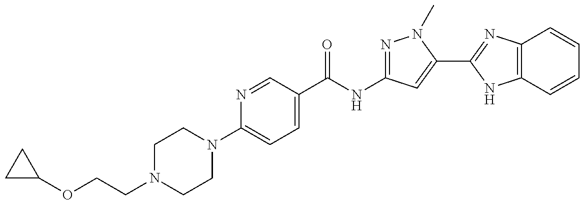 [SMILES: Cn1nc(NC(=O)c2ccc(N3CCN(CCOC4CC4)CC3)nc2)cc1-c1nc2ccccc2[nH]1] | N-[5-(1H-benzimidazol-2-yl)-1-methyl- pyrazol-3-yl]-6-[4-[2-(cyclo- propoxy)ethyl]piperazin-1-yl]pyridine- 3-carboxamide | A-266 |
| 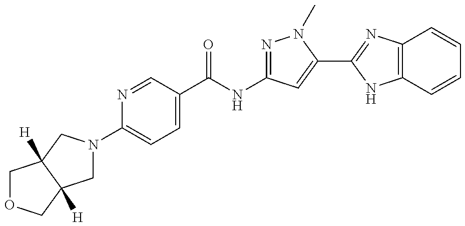 [SMILES: Cn1nc(NC(=O)c2ccc(N3C[C@H]4COC[C@H]4C3)nc2)cc1-c1nc2ccccc2[nH]1] | 6-[(3aR,6aS)-1,3,3a,4,6,6a- hexahydrofuro[3,4-c]pyrrol-5-yl]-N-[5-(1H- benzimidazol-2-yl)-1-methyl-pyrazol-3- yl]pyridine-3-carboxamide | A-267 |
| 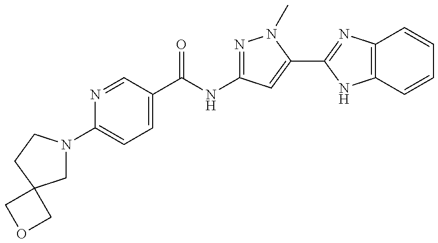 [SMILES: Cn1nc(NC(=O)c2ccc(N3CCC4(COC4)C3)nc2)cc1-c1nc2ccccc2[nH]1] | N-[5-(1H-benzimidazol-2-yl)-1-methyl- pyrazol-3-yl]-6-(2-oxa-7-aza- spiro[3.4]octan- 7-yl)pyridine-3-carboxamide | A-268 |
| 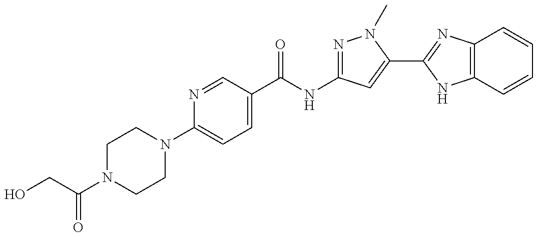 [SMILES: Cn1nc(NC(=O)c2ccc(N3CCN(C(=O)CO)CC3)nc2)cc1-c1nc2ccccc2[nH]1] | N-[5-(1H-benzimidazol-2-yl)-1-methyl- pyrazol-3-yl]-6-[4-(2- hydroxyacetyl)piperazin-1-yl]pyridine-3- carboxamide | A-269 |
| 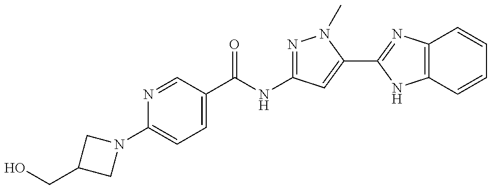 [SMILES: Cn1nc(NC(=O)c2ccc(N3CC(CO)C3)nc2)cc1-c1nc2ccccc2[nH]1] | N-[5-(1H-benzimidazol-2-yl)-1-methyl- pyrazol-3-yl]-6-[3-(hydroxymethyl)azetidin- 1-yl]pyridine-3-carboxamide | A-270 |
| 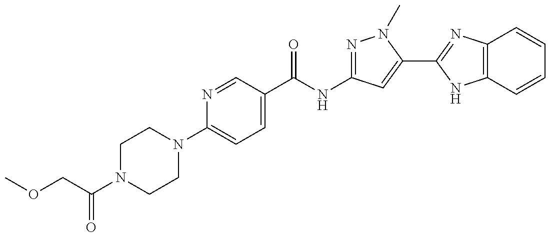 [SMILES: COCC(=O)N1CCN(c2ccc(C(=O)Nc3cc(-c4nc5ccccc5[nH]4)n(C)n3)cn2)CC1] | N-[5-(1H-benzimidazol-2-yl)-1-methyl- pyrazol-3-yl]-6-[4-(2- hydroxyacetyl)piperazin-1-yl]pyridine-3- carboxamide | A-271 |
| 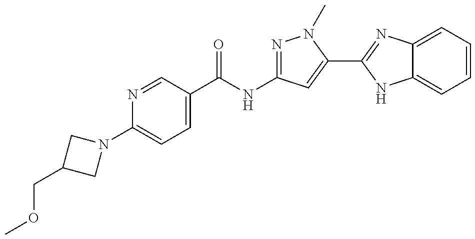 [SMILES: COCC1CN(c2ccc(C(=O)Nc3cc(-c4nc5ccccc5[nH]4)n(C)n3)cn2)C1] | N-[5-(1H-benzimidazol-2-yl)-1-methyl- pyrazol-3-yl]-6-[3-(methoxy- methyl)azetidin- 1-yl]pyridine-3-carboxamide | A-272 |
| 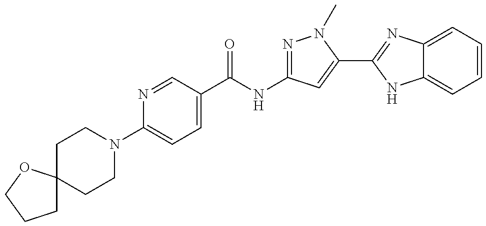 [SMILES: Cn1nc(NC(=O)c2ccc(N3CCC4(CCCO4)CC3)nc2)cc1-c1nc2ccccc2[nH]1] | N-[5-(1H-benzimidazol-2-yl)-1-methyl- pyrazol-3-yl]-6-(1-oxa-8-aza- spiro[4.5]decan- 8-yl)pyridine-3-carboxamide | A-273 |
| 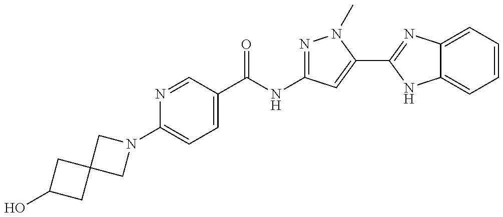 [SMILES: Cn1nc(NC(=O)c2ccc(N3CC4(CC(O)C4)C3)nc2)cc1-c1nc2ccccc2[nH]1] | N-[5-(1H-benzimidazol-2-yl)-1-methyl- pyrazol-3-yl]-6-(6-hydroxy-2- azaspiro[3.3]heptan-2-yl)pyridine-3- carboxamide | A-274 |
| 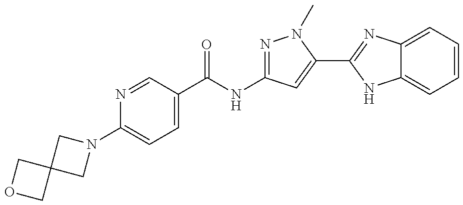 [SMILES: Cn1nc(NC(=O)c2ccc(N3CC4(COC4)C3)nc2)cc1-c1nc2ccccc2[nH]1] | N-[5-(1H-benzimidazol-2-yl)-1-methyl- pyrazol-3-yl]-6-(2-oxa-6- azaspiro[3.3]heptan- 6-yl)pyridine-3-carboxamide | A-275 |
| 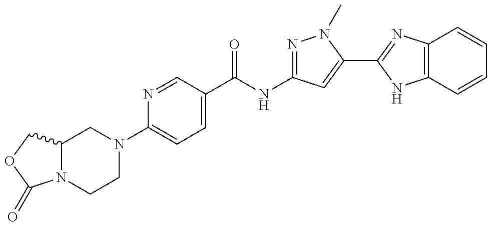 [SMILES: Cn1nc(NC(=O)c2ccc(N3CCN4C(=O)OCC4C3)nc2)cc1-c1nc2ccccc2[nH]1] | N-[5-(1H-benzimidazol-2-yl)-1-methyl- pyrazol-3-yl]-6-(3-oxo-5,6,8,8a-tetrahydro- 1H-oxazolo[3,4-a]pyrazin-7-yl)pyridine-3- carboxamide | A-276 |
| 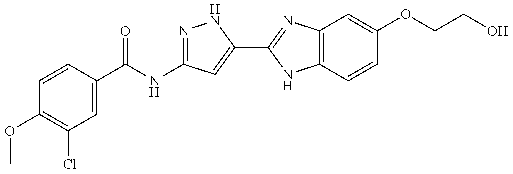 [SMILES: COc1ccc(C(=O)Nc2cc(-c3nc4cc(OCCO)ccc4[nH]3)[nH]n2)cc1Cl] | 3-chloro-N-[5-[5-(2-hydroxyethoxy)-1H- benzimidazol-2-yl]-1H-pyrazol-3-yl]-4- methoxy-benzamide | A-277 |
| 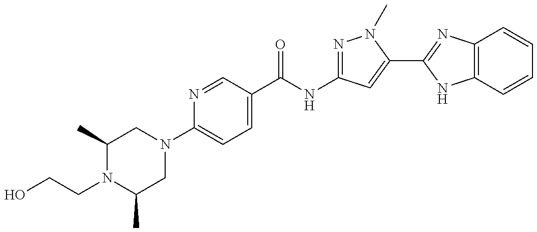 [SMILES: C[C@@H]1CN(c2ccc(C(=O)Nc3cc(-c4nc5ccccc5[nH]4)n(C)n3)cn2)C[C@H](C)N1CCO] | N-[5-(1H-benzimidazol-2-yl)-1-methyl- pyrazol-3-yl]-6-[(3R,5S)-4-(2-hydroxy- ethyl)-3,5-dimethyl-piperazin-1- yl]pyridine-3-carboxamide | A-278 |
| 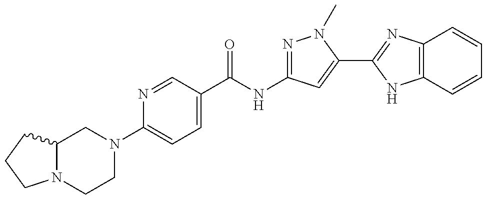 [SMILES: Cn1nc(NC(=O)c2ccc(N3CCN4CCCC4C3)nc2)cc1-c1nc2ccccc2[nH]1] | 6-(3,4,6,7,8,8a-hexahydro-1H-pyrrolo[1,2- a]pyrazin-2-yl)-N-[5-(1H-benzimidazol-2- yl)-1-methyl-pyrazol-3-yl]pyridine-3- carboxamide | A-279 |
| 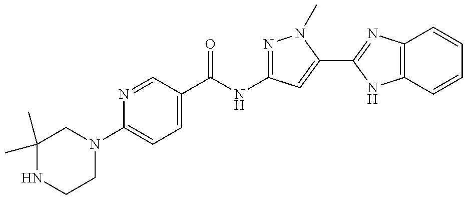 [SMILES: Cn1nc(NC(=O)c2ccc(N3CCNC(C)(C)C3)nc2)cc1-c1nc2ccccc2[nH]1] | N-[5-(1H-benzimidazol-2-yl)-1-methyl- pyrazol-3-yl]-6-(3,3-dimethylpiperazin-1- yl)pyridine-3-carboxamide | A-280 |
| 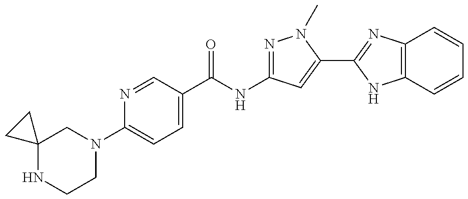 [SMILES: Cn1nc(NC(=O)c2ccc(N3CCNC4(CC4)C3)nc2)cc1-c1nc2ccccc2[nH]1] | N-[5-(1H-benzimidazol-2-yl)-1-methyl- pyrazol-3-yl]-6-(4,7-diazaspiro[2.5]octan-7- yl)pyridine-3-carboxamide | A-281 |
| 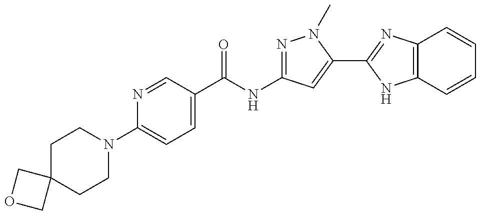 [SMILES: Cn1nc(NC(=O)c2ccc(N3CCC4(CC3)COC4)nc2)cc1-c1nc2ccccc2[nH]1] | N-[5-(1H-benzimidazol-2-yl)-1-methyl- pyrazol-3-yl]-6-(2-oxa-7-aza- spiro[3.5]nonan-7-yl)pyridine-3- carboxamide | A-282 |
| 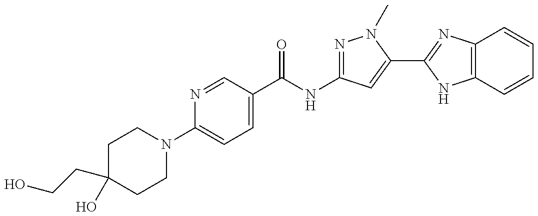 [SMILES: Cn1nc(NC(=O)c2ccc(N3CCC(O)(CCO)CC3)nc2)cc1-c1nc2ccccc2[nH]1] | N-[5-(1H-benzimidazol-2-yl)-1-methyl- pyrazol-3-yl]-6-[4-hydroxy-4-(2- hydroxyethyl)-1-piperidyl]pyridine-3- carboxamide | A-283 |
| 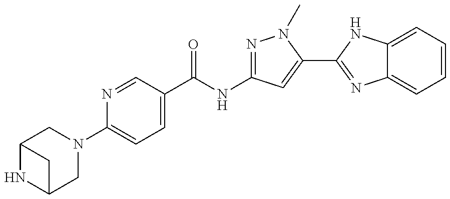 [SMILES: Cn1nc(NC(=O)c2ccc(N3CC4CC(C3)N4)nc2)cc1-c1nc2ccccc2[nH]1] | N-[5-(1H-benzimidazol-2-yl)-1-methyl- pyrazol-3-yl]-6-(3,6- diazabicyclo[3.1.1]heptan-3-yl)pyridine-3- carboxamide | A-284 |
| 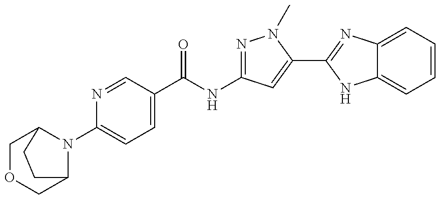 [SMILES: Cn1nc(NC(=O)c2ccc(N3C4CCC3COC4)nc2)cc1-c1nc2ccccc2[nH]1] | N-[5-(1H-benzimidazol-2-yl)-1-methyl- pyrazol-3-yl]-6-(3-oxa-8- azabicyclo[3.2.1]octan-8-yl)pyridine-3- carboxamide | A-285 |
| 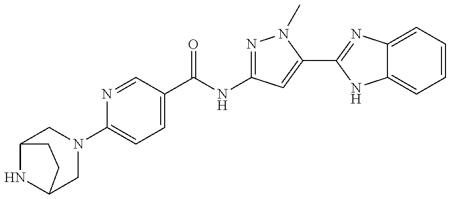 [SMILES: Cn1nc(NC(=O)c2ccc(N3CC4CCC(C3)N4)nc2)cc1-c1nc2ccccc2[nH]1] | N-[5-(1H-benzimidazol-2-yl)-1-methyl- pyrazol-3-yl]-6-(3,8- diazabicyclo[3.2.1]octan-3-yl)pyridine-3- carboxamide | A-286 |
| 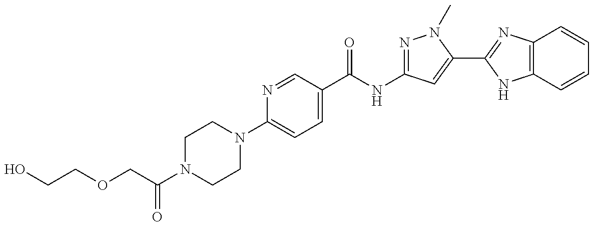 [SMILES: Cn1nc(NC(=O)c2ccc(N3CCN(C(=O)COCCO)CC3)nc2)cc1-c1nc2ccccc2[nH]1] | N-[5-(1H-benzimidazol-2-yl)-1-methyl- pyrazol-3-yl]-6-[4-[2-(2- hydroxyethoxy)acetyl]piperazin-1- yl]pyridine-3-carboxamide | A-287 |
| 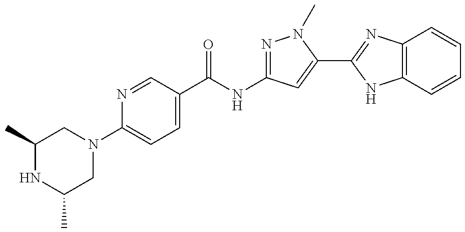 [SMILES: C[C@H]1CN(c2ccc(C(=O)Nc3cc(-c4nc5ccccc5[nH]4)n(C)n3)cn2)C[C@H](C)N1] | N-[5-(1H-benzimidazol-2-yl)-1-methyl- pyrazol-3-yl]-6-[(3S,5S)-3,5- dimethylpiperazin-1-yl]pyridine-3- carboxamide | A-288 |
| 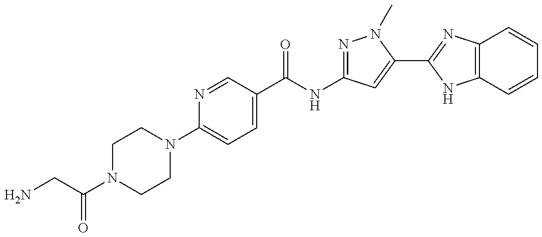 [SMILES: Cn1nc(NC(=O)c2ccc(N3CCN(C(=O)CN)CC3)nc2)cc1-c1nc2ccccc2[nH]1] | 6-[4-(2-aminoacetyl)piperazin-1-yl]-N-[5- (1H-benzimidazol-2-yl)-1-methyl-pyrazol- 3-yl]pyridine-3-carboxamide | A-289 |
| 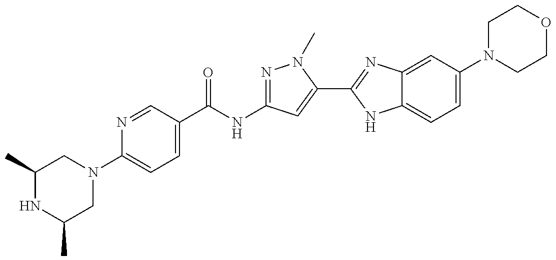 [SMILES: C[C@@H]1CN(c2ccc(C(=O)Nc3cc(-c4nc5cc(N6CCOCC6)ccc5[nH]4)n(C)n3)cn2)C[C@H](C)N1] | 6-[(3R,5S)-3,5-dimethylpiperazin-1- yl]-N-[1-methyl-5-(5-morpholino-1H- benzimidazol-2-yl)pyrazol-3- yl]pyridine-3-carboxamide | A-290 |
| 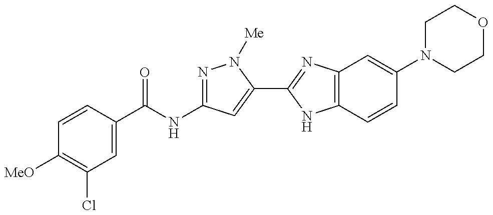 [SMILES: COc1ccc(C(=O)Nc2cc(-c3nc4cc(N5CCOCC5)ccc4[nH]3)n(C)n2)cc1Cl] | 3-chloro-4-methoxy-N-[1-methyl-5-(5- morpholino-1H-benzimidazol-2-yl)pyrazol- 3-yl]benzamide | A-291 |
| 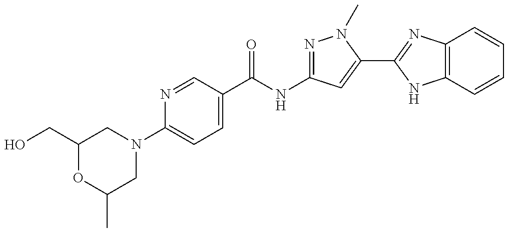 [SMILES: CC1CN(c2ccc(C(=O)Nc3cc(-c4nc5ccccc5[nH]4)n(C)n3)cn2)CC(CO)O1] | N-[5-(1H-benzimidazol-2-yl)-1-methyl- pyrazol-3-yl]-6-[2-(hydroxymethyl)-6- methyl-morpholin-4-yl]pyridine-3- carboxamide - isomer 1 | A-292 |
| 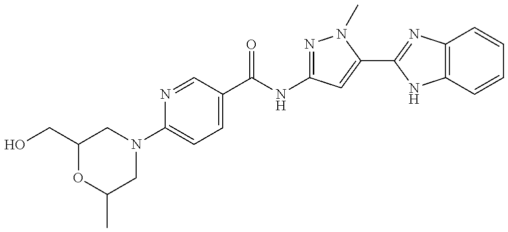 [SMILES: CC1CN(c2ccc(C(=O)Nc3cc(-c4nc5ccccc5[nH]4)n(C)n3)cn2)CC(CO)O1] | N-[5-(1H-benzimidazol-2-yl)-1-methyl- pyrazol-3-yl]-6-[2-(hydroxymethyl)-6- methyl-morpholin-4-yl]pyridine-3- carboxamide - isomer 2 | A-293 |
| 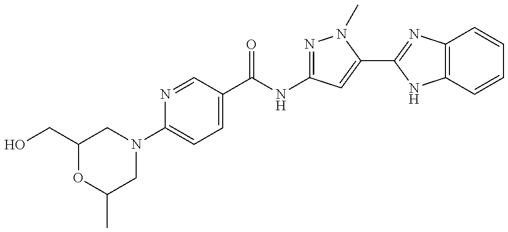 [SMILES: CC1CN(c2ccc(C(=O)Nc3cc(-c4nc5ccccc5[nH]4)n(C)n3)cn2)CC(CO)O1] | N-[5-(1H-benzimidazol-2-yl)-1-methyl- pyrazol-3-yl]-6-[2-(hydroxymethyl)-6- methyl-morpholin-4-yl]pyridine-3- carboxamide - isomer 3 | A-294 |
| 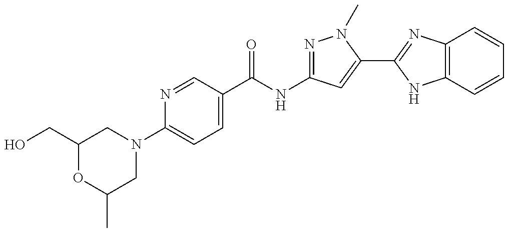 [SMILES: CC1CN(c2ccc(C(=O)Nc3cc(-c4nc5ccccc5[nH]4)n(C)n3)cn2)CC(CO)O1] | N-[5-(1H-benzimidazol-2-yl)-1-methyl- pyrazol-3-yl]-6-[2-(hydroxymethyl)-6- methyl-morpholin-4-yl]pyridine-3- carboxamide - isomer 4 | A-295 |

### EXAMPLE A

### Inhibitor Activity Assay

The inhibitory activities of compounds on FOXO1 were determined by a cell-based transcriptional reporter assay. HEK293 cells were plated onto either 384-well poly-D-lysine coated plates (at 7500 cells per well) or 96-well plates (at 20000 cells per well) in low glucose Eagle's Minimum Essential Medium (EMEM) containing 1% fetal bovine serum, and incubated overnight at 37° C. and 5% CO₂. Cells were then transfected using Lipofectamine 3000™ (Thermo Fisher Scientific) according to manufacturer's protocol with the following DNA plasmids in each well: (1) pGL4.26 containing 4 tandem copies of an insulin response element (each copy with the sequence of 5′-GCAAAACAAACTTATTTTGAA-3′) upstream of a firefly luciferase reporter: 12.5 ng per well for 384-well plates or 50 ng per well for 96-well plates, (2) pcDNA3.1 containing a constitutively active form of human FOXO1 (Thr24Ala, Ser256Ala, Ser319Ala) with an in-frame FLAG epitope at the 3′ end: 1.25 ng per well for 384-well plates or 5 ng per well for 96-well plates, (3) pRL-CMV encoding constitutively expressed Renilla luciferase: 1.25 ng per well for 384-well plates or 5 ng per well for 96-well plates. Compounds were then added at varying final concentrations in a 10-point dose response with 3-fold serial dilutions (ranging from 50 μM to 2.5 nM) with a final DMSO concentration of 0.5% in each well. Duplicate wells were included for each treatment condition. Cells were incubated for 24 hours at 37° C. and 5% CO₂. Luciferase activities in each well were measured using Dual-Glog Luciferase Assay System (Promega) according to manufacturer's protocol and an Envision plate reader suitable for luminescence detection. Firefly luciferase activity was divided by Renilla luciferase activity to calculate a ratio for each well. The ratio in wells containing cells transfected with all 3 plasmids listed above and receiving only DMSO without compound addition was set to 100%. The ratio in wells containing cells transfected with plasmid (1), plasmid (3), and pcDNA3.1 containing the open reading frame of red fluorescent protein (instead of human FOXO1) and treated with DMSO was set to 0%. The ratio in each well receiving compound treatment was normalized and expressed as a percentage. Data were fit by 4-parameter logistic regression to determine IC50 and maximal inhibition values. Each compound was tested in a minimum of 2 independent experiments. The results are summarized in TABLE 2.

TABLE 2

| Cpd # | IC₅₀¹ |
|---|---|
| A-16 | * |
| A-21 | **** |
| A-29 | **** |
| A-30 | * |
| A-33 | *** |
| A-36 | **** |
| A-39 | **** |
| A-40 | **** |
| A-41 | **** |
| A-42 | *** |
| A-43 | **** |
| A-44 | **** |
| A-45 | * |
| A-46 | **** |
| A-47 | **** |
| A-48 | * |
| A-49 | ** |
| A-50 | **** |
| A-51 | * |
| A-52 | *** |
| A-53 | **** |
| A-54 | ** |
| A-55 | ** |
| A-56 | * |
| A-57 | * |
| A-58 | **** |
| A-59 | ** |
| A-60 | * |
| A-61 | *** |
| A-62 | * |
| A-63 | * |
| A-64 | * |
| A-65 | **** |
| A-66 | ** |
| A-67 | **** |
| A-68 | **** |
| A-69 | ** |
| A-70 | * |
| A-71 | ** |
| A-72 | * |
| A-73 | ** |
| A-74 | * |
| A-75 | **** |
| A-76 | * |
| A-77 | **** |
| A-78 | *** |
| A-79 | **** |
| A-80 | **** |
| A-81 | *** |
| A-82 | **** |
| A-83 | **** |
| A-84 | *** |
| A-85 | **** |
| A-86 | * |
| A-87 | * |
| A-88 | * |
| A-89 | * |
| A-90 | * |
| A-91 | * |
| A-92 | * |
| A-93 | **** |
| A-94 | **** |
| A-95 | *** |
| A-96 | **** |
| A-97 | *** |
| A-98 | **** |
| A-99 | *** |
| A-100 | *** |
| A-101 | * |
| A-102 | * |
| A-103 | **** |
| A-104 | **** |
| A-105 | * |
| A-106 | *** |
| A-107 | * |
| A-108 | * |
| A-109 | **** |
| A-111 | * |
| A-112 | ** |
| A-113 | **** |
| A-114 | * |
| A-115 | **** |
| A-116 | *** |
| A-117 | **** |
| A-118 | ** |
| A-119 | *** |
| A-120 | ** |
| A-121 | *** |
| A-122 | * |
| A-123 | **** |
| A-124 | **** |
| A-125 | *** |
| A-126 | **** |
| A-127 | **** |
| A-128 | ** |
| A-129 | *** |
| A-130 | ** |
| A-131 | *** |
| A-132 | **** |
| A-133 | *** |
| A-134 | *** |
| A-135 | *** |
| A-136 | *** |
| A-137 | ** |
| A-138 | * |
| A-139 | *** |
| A-140 | * |
| A-141 | *** |
| A-142 | **** |
| A-143 | *** |
| A-144 | * |
| A-145 | * |
| A-146 | * |
| A-147 | * |
| A-148 | *** |
| A-149 | *** |
| A-150 | *** |
| A-151 | * |
| A-152 | **** |
| A-153 | *** |
| A-154 | *** |
| A-155 | * |
| A-156 | * |
| A-157 | *** |
| A-158 | *** |
| A-159 | **** |
| A-160 | ** |
| A-161 | *** |
| A-162 | * |
| A-163 | ** |
| A-164 | **** |
| A-165 | **** |
| A-166 | **** |
| A-167 | **** |
| A-168 | *** |
| A-169 | *** |
| A-170 | *** |
| A-171 | **** |
| A-172 | **** |
| A-173 | *** |
| A-174 | **** |
| A-175 | * |
| A-176 | * |
| A-177 | * |
| A-178 | ** |
| A-179 | *** |
| A-180 | *** |
| A-181 | **** |
| A-182 | ** |
| A-183 | * |
| A-184 | * |
| A-185 | * |
| A-186 | * |
| A-187 | ** |
| A-188 | * |
| A-189 | **** |
| A-190 | **** |
| A-191 | *** |
| A-192 | * |
| A-193 | * |
| A-194 | * |
| A-195 | *** |
| A-196 | * |
| A-197 | * |
| A-198 | ** |
| A-199 | *** |
| A-200 | ** |
| A-201 | ** |
| A-202 | **** |
| A-203 | *** |
| A-204 | *** |
| A-205 | ** |
| A-206 | * |
| A-207 | ** |
| A-208 | *** |
| A-209 | **** |
| A-210 | **** |
| A-211 | *** |
| A-212 | **** |
| A-213 | *** |
| A-214 | **** |
| A-215 | *** |
| A-216 | *** |
| A-217 | *** |
| A-218 | *** |
| A-219 | **** |
| A-220 | **** |
| A-221 | ** |
| A-222 | **** |
| A-223 | **** |
| A-224 | *** |
| A-225 | ** |
| A-226 | *** |
| A-227 | ** |
| A-228 | *** |
| A-229 | *** |
| A-230 | *** |
| A-231 | **** |
| A-232 | * |
| A-233 | ** |
| A-234 | ** |
| A-235 | *** |
| A-236 | *** |
| A-237 | **** |
| A-238 | *** |
| A-239 | * |
| A-240 | **** |
| A-241 | ** |
| A-242 | *** |
| A-243 | ** |
| A-244 | *** |
| A-245 | **** |
| A-246 | *** |
| A-247 | **** |
| A-248 | **** |
| A-249 | *** |
| A-250 | ** |
| A-251 | **** |
| A-252 | **** |
| A-253 | *** |
| A-254 | * |
| A-255 | **** |
| A-256 | **** |
| A-257 | **** |
| A-258 | ** |
| A-259 | **** |
| A-260 | *** |
| A-261 | **** |
| A-262 | *** |
| A-263 | *** |
| A-264 | ** |
| A-265 | **** |
| A-266 | **** |
| A-267 | **** |
| A-268 | *** |
| A-269 | *** |
| A-270 | ** |
| A-271 | *** |
| A-272 | **** |
| A-273 | *** |
| A-274 | *** |
| A-275 | *** |
| A-276 | **** |
| A-277 | **** |
| A-278 | *** |
| A-279 | **** |
| A-280 | **** |
| A-281 | **** |
| A-282 | **** |
| A-283 | **** |
| A-284 | *** |
| A-285 | **** |
| A-286 | *** |
| A-287 | ** |
| A-288 | *** |
| A-289 | *** |
| A-290 | **** |
| A-291 | **** |
| A-292 | **** |
| A-293 | *** |
| A-294 | **** |
| A-295 | **** |

¹Key: **** ≤ 100 nM; 100 nM < *** ≤ 300 nM; 300 nM < ** ≤ 1 μM; * > 1 μM

### EXAMPLE B

### Preparation of Compounds and Selected Synthetic Intermediates

### General Methods.

All temperatures are in degrees Celsius (° C.) and are uncorrected. Reagent grade chemicals and anhydrous solvent were purchased from commercial sources and, unless otherwise mentioned, were used without further purification. Silica gel chromatography was performed on Teledyne ISCO™ instruments using pre-packaged disposable SiO₂ stationary phase columns with eluent flow rate range of 15 to 200 mL/min, UV detection (254 and 280 nm). Reverse phase preparative HPLC was carried out using C18 columns, UV detection (214 and 254 nm) eluting with gradients of MeCN in H₂O (10 mM (NH₄)(HCO₃) or 10 mM (NH₄)(HCO₂)). The analytical HPLC chromatograms were performed using an Agilent 1100 Series™ instrument with DAD detector (190 nm to 300 nm). The mass spectra were recorded with a Waters Micromass ZQ™ detector at 130° C. The mass spectrometer was equipped with an electrospray ion source (ESI) operated in a positive ion mode and was set to scan between m/z 150-750 with a scan time of 0.3 s. Products and intermediates were analyzed by HPLC/MS on a Gemini-NX™ (5 uM, 2.0×30 mm) using a high pH buffer gradient of 5% to 100% of MeCN in H₂O (10 mM (NH₄)(HCO₃) over 2.5 min at 1.8 mL/min for a 3.5 min run (B05) and EVO C18™ (5 uM, 3.0×50 mm) using a low pH buffer gradient of 5% to 100% of MeCN in H₂O (10 mM (NH₄)(HCO₂)) over 2.5 min at 2.2 mL/min for a 3.5 min run (A05). The ¹H NMR spectra were recorded on a Varian AS™8 400 MHz/54 mm instrument and a Bruker UltraShield™ 500 MHz/54 mm instrument (BZH 43/500/70B, D221/54-3209). The chemical shifts are referenced to solvent peaks, which in ¹H NMR appear at 7.26 ppm for CDCl₃, 2.51 for DMSO-d6, and 3.33 ppm for CD₃OD. The chemical shifts of rotamers or tautomers are listed as major [minor].

### Terms and Abbreviations.

AcOH acetic acid;
aq aqueous;
Cs₂CO₃ cesium carbonate;
DCM dichloromethane;
DIEA N,N-diisopropylethylamine
DMF N,N-dimethyl formamide;
DMSO dimethyl sulfoxide;
Et₂O diethyl ether;
EtOAc ethyl acetate;
EtOH ethanol;
h hour(s);
HATU 2-(1H-benzotriazol-1-yl)-1,1,3,3-tetramethyluronium
hexafluorophosphate;
HCl hydrochloride;
HPLC high performance liquid chromatography;
K₂CO₃ potassium carbonate;
LCMS liquid chromatography mass spectrometry;
LiOH lithium hydroxide;
MeI iodomethane;
MeCN acetonitrile;
MeOH methanol;
MgSO₄ magnesium sulfate
min minute(s);
MS mass spectrometry;
n-BuLi n-butyllithium;
NaHCO₃ sodium bicarbonate;
Na₂SO₄ sodium sulfate
NEt₃ triethylamine
NH₄Cl ammonium chloride;
NMR nuclear magnetic resonance;
PBr₃ phosphorus tribromide;
Pd(OAc)₂ palladium(II) acetate
PhMe toluene;
PMB para-methoxy benzyl;
p-TsOH—H₂O para-toluenesulfonic acid monohydrate
sat. saturated;
SOCl₂ thionyl chloride;
THF tetrahydrofuran;

### EXAMPLE B1

### Preparation of Compounds J-1 to J-10

### Representative Synthetic Scheme for the Syntheses of Compounds J-1 to J-10.

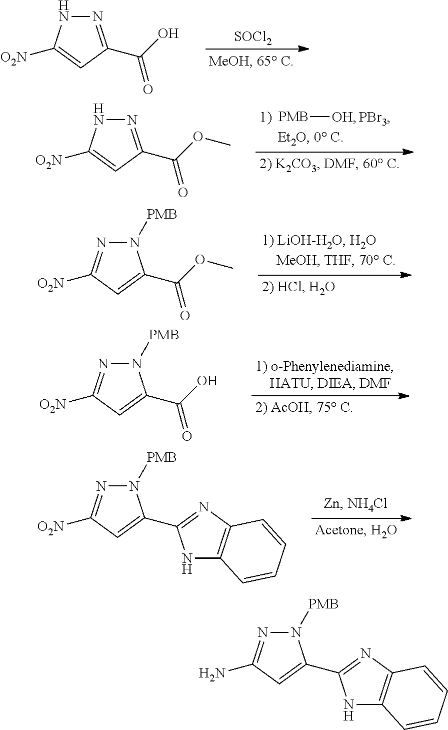

### Preparation of Compound J-1.

### Step 1: methyl 5-nitro-1H-pyrazole-3-carboxylate

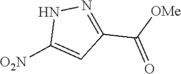 [SMILES: COC(=O)c1cc([N+](=O)[O-])[nH]n1]

SOCl₂ (60 mL, 827 mmol) was added dropwise to a solution of 5-nitro-1H-pyrazole-3-carboxylic acid (50.0 g, 318 mmol) in MeOH (340 mL) at 0° C. The mixture was heated at reflux for 2.5 h, cooled to 23° C., and concentrated. Acetone (200 mL) was added. The mixture was concentrated. The residue was diluted in hexanes (250 mL), and the mixture was filtered. The solid was dried under high vacuum to provide the title compound as a solid (53.8 g, 99%). ¹H NMR (500 MHz, DMSO) δ 15.24 (s, 1H), 7.54 (s, 1H), 3.91 (s, 3H). m/z (ES), [M−H]⁻ 170.0. HPLC (A05) t_{R}=1.74 min.

### Step 2: methyl 2-[(4-methoxyphenyl)methyl]-5-nitro-pyrazole-3-carboxylate

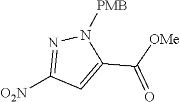

PBr₃ (22.1 mL, 235 mmol) was added dropwise to a solution of 4-methoxybenzyl alcohol (65.0 g, 471 mmol) in Et₂O (600 mL) at 0° C. The mixture was warmed to 23° C., stirred for 1 h, and poured slowly into sat. NaHCO₃/ice (500 mL). The organic layer was separated, and the aq. phase was extracted with Et₂O (500 mL). The combined organic phases were washed with brine (500 mL), dried (MgSO₄), filtered, and concentrated. The residue was dissolved in DMF (100 mL), and the mixture was added to a mixture of methyl 5-nitro-1H-pyrazole-3-carboxylate (53.7 g, 314 mmol) and K₂CO₃ (75.9 g, 549 mmol) in DMF (1.30 L). The mixture was heated at 60° C. for 7 h. The mixture was cooled to 23° C., and brine (4.00 L) was added. The aq. phase was extracted with Et₂O (1×2 L, 1×1 L). The combined organic phases were washed with brine (1 L), dried (MgSO₄), filtered, and concentrated. The residue was diluted with MeOH (125 mL), and the mixture was cooled to 0° C. The mixture was filtered, and the solid was washed with hexanes and dried under high vacuum to provide the title compound as a solid (60.7 g, 66%). ¹H NMR (500 MHz, CDCl₃) δ 7.39 (s, 1H), 7.37-7.32 (m, 2H), 6.87-6.83 (m, 2H), 5.76 (s, 2H), 3.92 (s, 3H), 3.78 (s, 3H). m/z (ES⁺), No ionization. HPLC (A05) t_{R}=2.46 min.

### Step 3: 2-[(4-methoxyphenyl)methyl]-5-nitro-pyrazole-3-carboxylic acid

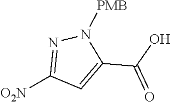

LiOH—H₂O (9.62 g, 229 mmol) was added to a mixture of methyl 2-[(4-methoxyphenyl)methyl]-5-nitro-pyrazole-3-carboxylate (55.7 g, 191 mmol) in MeOH (200 mL), THF (200 mL), and water (30 mL). The mixture was heated at 70° C. for 1.5 h. After cooling to 23° C., the mixture was concentrated. Water (600 mL) was added, and HCl (12 M, 17.5 mL) followed by HCl (1M, 30 mL) were slowly added. The mixture was filtered, and the solid was washed with water and dried under high vacuum to provide the title compound as a solid (51.6 g, 97%). ¹H NMR (500 MHz, DMSO) δ 14.31 (br, 1H), 7.50 (s, 1H), 7.29-7.21 (m, 2H), 6.95-6.89 (m, 2H), 5.76 (s, 2H), 3.73 (s, 3H). m/z (ES⁺), [M−H]⁻ 276.2. HPLC (A05) t_{R}=1.86 min.

### Step 4: 2-[2-[(4-methoxyphenyl)methyl]-5-nitro-pyrazol-3-yl]-1H-benzimidazole

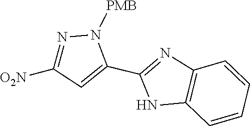

DIEA (32 mL, 184 mmol) was added to a solution of HATU (21.3 g, 56.1 mmol) and 2-[(4-methoxyphenyl)methyl]-5-nitro-pyrazole-3-carboxylic acid (14.2 g, 51.0 mmol) in DMF (250 mL) at 0° C. The mixture was stirred at 0° C. for 1 h. DMF (200 mL) was added, followed by a solution of o-phenylenediamine (5.80 g, 53.6 mmol) in DMF (50 mL). The mixture was warmed to 23° C., stirred for 3 h, and poured into water (1.00 L). The aq. phase was extracted with EtOAc (3×500 mL). The combined organic phases were washed with brine (500 mL), dried (MgSO₄), filtered, and concentrated. AcOH (250 mL) was added to the residue (m/z (ES⁺), [M+H]⁺ 368.3. HPLC (A05) t_{R}=2.38 min), and the mixture was heated at 75° C. for 1.5 h. After cooling to 23° C., the mixture was poured into water (2 L). After stirring at 23° C. for 10 min, the solid was filtered, washed with water, and dried in a vacuum oven at 65° C. for 5 h to provide the title compound as a solid (10.2 g, 58%). ¹H NMR (500 MHz, DMSO) δ 13.29 (s, 1H), 7.81 (d, J=8.1 Hz, 1H), 7.78 (s, 1H), 7.63 (d, J=7.9 Hz, 1H), 7.39-7.33 (m, 3H), 7.33-7.28 (m, 1H), 6.91-6.86 (m, 2H), 6.19 (s, 2H), 3.70 (s, 3H). m/z (ES⁺), [M+H]⁺ 350.2. HPLC (A05) t_{R}=2.54 min.

### Step 5: 5-(1H-benzimidazol-2-yl)-1-[(4-methoxyphenyl)methyl]pyrazol-3-amine

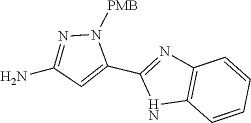

Zinc dust (9.58 g, 147 mmol) was added in portions to a mixture of 2-[2-[(4-methoxyphenyl)methyl]-5-nitro-pyrazol-3-yl]-1H-benzimidazole (10.2 g, 29.3 mmol) and NH₄Cl (15.7 g, 293 mmol) in acetone (320 mL) and water (65 mL). The internal temperature was kept between 20-23° C. during the addition by cooling with an ice bath when required. The suspension was stirred at 23° C. for 4 h. The mixture was filtered through Celite, washing with acetone. The filtrate was concentrated. Water (250 mL) and sat. aq. Rochelle salt (100 mL) were added to the residue, and the aq. phase was extracted with EtOAc (3×500 mL). The combined organic phases were washed with brine (500 mL), dried (MgSO₄), filtered, and concentrated. The residue was diluted with Et₂O (250 mL). After 10 min, the mixture was filtered, and the solid was washed with hexanes and dried under high vacuum to provide the title compound as a solid (5.76 g, 62%). ¹H NMR (400 MHz, DMSO) δ 12.77 (s, 1H), 7.68 (d, J=7.8 Hz, 1H), 7.51 (d, J=7.5 Hz, 1H), 7.31-7.08 (m, 4H), 6.89-6.69 (m, 2H), 6.20 (s, 1H), 5.79 (s, 2H), 4.88 (s, 2H), 3.68 (s, 3H). m/z (ES⁺), [M+H]⁺ 320.3. HPLC (A05) t_{R}=2.14 min.

### Preparation of Compounds J-2 to J-10.

Compounds J-2 to J-10, as depicted in TABLE 3 below, were synthesized via procedures analogous to the above detailed synthesis of Compound J-1.

TABLE 3

| Structure | Characterization | Cpd # |
|---|---|---|
| 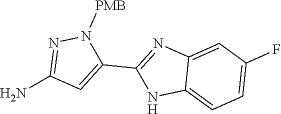 | 5-(5-fluoro-1H-benzimidazol-2-yl)-1-[(4- methoxyphenyl)methyl]pyrazol-3-amine ¹H NMR (400 MHz, CD₃OD) δ 7.58 (s, 1H), 7.29 (s, 1H), 7.08-7.05 (m, 3H), 6.74 (d, J = 8.7 Hz, 2H), 6.20 (s, 1H), 5.69 (s, 2H), 3.69 (s, 3H). m/z (ES⁺), [M + H]⁺ 338.3. | J-2 |
| 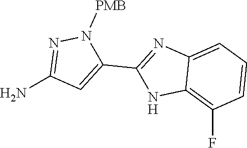 | 5-(7-fluoro-1H-benzimidazol-2-yl)-1-[(4- methoxyphenyl)methyl]pyrazol-3-amine ¹H NMR (400 MHz, CD₃OD) δ 7.36 (d, J = 8.0 Hz, 1H), 7.22 (td, J = 8.1, 4.8 Hz, 1H), 7.10 (d, J = 8.8 Hz, 2H), 6.98 (dd, J = 11.0, 7.8 Hz, 1H), 6.74 (d, J = 8.8 Hz, 2H), 6.23 (s, 1H), 5.71 (s, 2H), 3.69 (s, 3H). m/z (ES⁺), [M + H]⁺ 338.3. | J-3 |
| 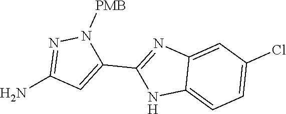 | 5-(5-chloro-1H-benzimidazol-2-yl)-1-[(4- methoxyphenyl)methyl]pyrazol-3-amine ¹H NMR (400 MHz, CD₃OD) δ 7.57 (s, 2H), 7.25 (dd, J = 8.6, 1.9 Hz, 1H), 7.07 (d, J = 8.8 Hz, 2H), 6.74 (d, J = 8.8 Hz, 2H), 6.22 (s, 1H), 5.70 (s, 2H), 3.69 (s, 3H). m/z (ES⁺), [M + H]⁺ 354.8. | J-4 |
| 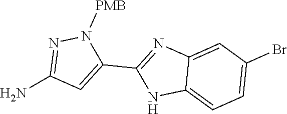 | 5-(5-bromo-1H-benzimidazol-2-yl)-1-[(4- methoxyphenyl)methyl]pyrazol-3-amine ¹H NMR (400 MHz, DMSO) δ 12.98 (s, 1H), 7.80 (s, 1H), 7.53 (s, 1H), 7.36 (d, J = 8.2 Hz, 1H), 7.16 (d, J = 8.6 Hz, 2H), 6.82 (d, J = 8.7 Hz, 2H), 6.21 (s, 1H), 5.76 (s, 2H), 4.92 (s, 2H), 3.68 (s, 3H). m/z (ES⁺), [M + H]⁺ 399.3. | J-5 |
| 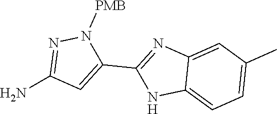 | l-[(4-methoxyphenyl)methyl]-5-(5-methyl-1H- benzimidazol-2-yl)pyrazol-3-amine ¹H NMR (400 MHz, DMSO) δ 12.62 (d, J = 11.3 Hz, 1H), 7.42 (dd, J = 73.5, 33.0 Hz, 2H), 7.16 (d, J = 8.5 Hz, 2H), 7.04 (s, 1H), 6.81 (d, J = 8.7 Hz, 2H), 6.17 (s, 1H), 5.77 (s, 2H), 4.88 (s, 2H), 3.68 (s, 3H), 2.42 (s, 3H). | J-6 |
| | m/z (ES⁺), [M + H]⁺ 334.4. | |
| 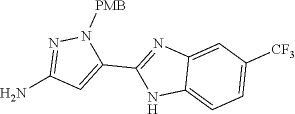 | l-[(4-methoxyphenyl)methyl]-5-[5- (trifluoromethyl)-1H-benzimidazol-2-yl]pyrazol-3- amine ¹H NMR (400 MHz, DMSO) δ 13.23 (s, 1H), 8.24- 7.67 (m, 2H), 7.55 (d, J = 8.2 Hz, 1H), 7.18 (d, J = 8.6 Hz, 2H), 6.82 (d, J = 11.4 Hz, 2H), 6.26 (s, 1H), 5.78 (s, 2H), 4.95 (s, 2H), 3.68 (s, 3H). | J-7 |
| | m/z (ES⁺), [M + H]⁺ 388.4. | |
| 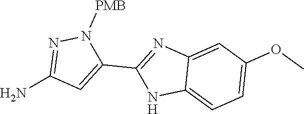 | 5-(5-methoxy-1H-benzimidazol-2-yl)-1-[(4- methoxyphenyl)methyl]pyrazol-3-amine ¹H NMR (400 MHz, DMSO) δ 12.62 (d, J = 12.7 Hz, 1H), 7.47 (dd, J = 70.4, 8.4 Hz, 1H), 7.18 (t, J = 13.3 Hz, 2H), 6.86 (t, J = 30.9 Hz, 4H), 6.15 (s, 1H), 5.76 (s, 2H), 4.86 (s, 2H), 3.80 (s, 3H), 3.68 (s, 3H). | J-8 |
| | m/z (ES⁺), [M + H]⁺ 350.5. | |
| 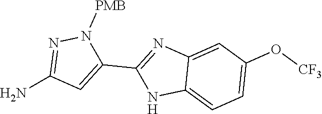 | l-[(4-methoxyphenyl)methyl]-5-[5- (trifluoromethoxy)-1H-benzimidazol-2-yl]pyrazol- 3-amine ¹H NMR (400 MHz, DMSO) δ 13.08 (s, 1H), 7.63 (dd, J = 84.2, 43.4 Hz, 2H), 7.19 (t, J = 13.7 Hz, 3H), 6.91-6.72 (m, 2H), 6.23 (s, 1H), 5.77 (s, 2H), 4.93 (s, 2H), 3.68 (s, 3H). | J-9 |
| | m/z (ES⁺), [M + H]⁺404.4. | |
| 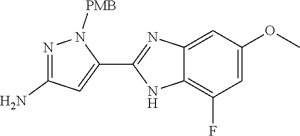 | 5-(7-fluoro-5-methoxy-1H-benzimidazol-2-yl)-1- [(4-methoxyphenyl)methyl]pyrazol-3-amine ¹H NMR (400 MHz, CD₃OD) δ 7.09 (d, J = 8.4 Hz, 2H), 6.81 (s, 1H), 6.78-6.71 (m, 2H), 6.65 (d, J = 12.2 Hz, 1H), 6.17 (s, 1H), 5.67 (s, 2H), 3.84 (s, 3H), 3.70 (s, 3H). m/z (ES⁺), [M + H]⁺ 368.4. | J-10 |

### EXAMPLE B2

### Preparation of Compounds J-11 to J-14

### Representative Synthetic Scheme for the Syntheses of Compounds J-11 to J-14.

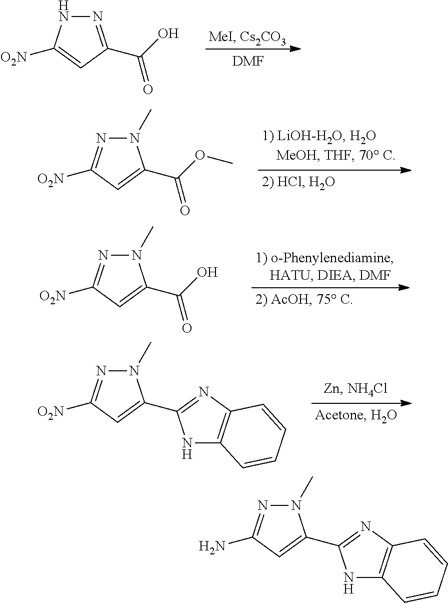

### Preparation of Compound J-11.

### Step 1: methyl 2-methyl-5-nitro-pyrazole-3-carboxylate

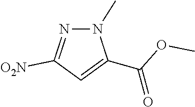

MeI (10.6 g, 75.0 mmol) was added at 23° C. to a mixture of 5-nitro-1H-pyrazole-3-carboxylic acid (4.71 g, 30.0 mmol) and Cs₂CO₃ (29.3 g, 90.0 mmol) in DMF (300 mL). The mixture was stirred at 23° C. for 3 days and poured into water (1.50 L). The aq. phase was extracted with EtOAc (2×1.00 L). The combined organic phases were dried (MgSO₄), filtered, and concentrated. The residue was purified by silica gel chromatography (120 g cartridge) eluting with hexanes and EtOAc (0-15%) to provide the title compound as a solid (2.78 g, 50%). ¹H NMR (500 MHz, CDCl₃) δ 7.39 (s, 1H), 4.28 (s, 3H), 3.95 (s, 3H). m/z (ES⁺), No ionization. HPLC (A05) t_{R}=1.98 min.

Note: methyl 1-methyl-5-nitro-pyrazole-3-carboxylate was also isolated as a solid (0.335 g, 6%). ¹H NMR (500 MHz, CDCl₃) δ 7.53 (s, 1H), 4.34 (s, 3H), 3.96 (s, 3H). m/z (ES⁺), No ionization. HPLC (A05) t_{R}=1.87 min.

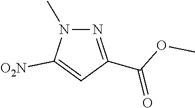

### Step 2: 2-methyl-5-nitro-pyrazole-3-carboxylic acid

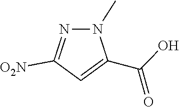

LiOH—H₂O (1.88 g, 44.7 mmol) was added to a solution of methyl 2-methyl-5-nitro-pyrazole-3-carboxylate (2.76 g, 14.9 mmol) in MeOH (40 mL), THF (40 mL), and water (15 mL). The mixture was stirred at 70° C. for 1 h and concentrated. Water (100 mL) was added, and the aq. phase was acidified to pH 1 with conc. HCl. The mixture was filtered, and the solid was dried in a vacuum oven at 45° C. for 18 h to provide the title compound as a solid (1.95 g, 76%). ¹H NMR (500 MHz, DMSO) δ 7.46 (s, 1H), 4.19 (s, 3H). m/z (ES⁻), [M−H]⁻ 169.9. HPLC (A05) t_{R}=0.42 min.

### Step 3: 2-(2-methyl-5-nitro-pyrazol-3-yl)-1H-benzimidazole

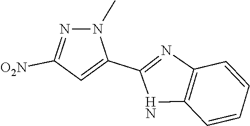 [SMILES: Cn1nc([N+](=O)[O-])cc1-c1nc2ccccc2[nH]1]

DIEA (6.91 mL, 39.7 mmol) was added to a solution of HATU (4.74 g, 12.5 mmol) and 2-methyl-5-nitro-pyrazole-3-carboxylic acid (1.94 g, 11.3 mmol) in DMF (30 mL) at 0° C. The mixture was stirred at 0° C. for 15 min. o-Phenylenediamine (1.29 g, 11.9 mmol) was added. The mixture was warmed to 23° C. and stirred for 18 h. Water (300 mL) was added, and the aq. phase was extracted with EtOAc (3×150 mL). The combined organic phases were washed with sat. NaHCO₃ (2×150 mL) and brine (150 mL), dried (MgSO₄), filtered, and concentrated. The residue (m/z (ES⁺), [M+H]⁺ 262.2. HPLC (A05) t_{R}=1.99 min) was dissolved in AcOH (30 mL) and stirred at 70° C. for 1 h. The mixture was concentrated. The residue was diluted with EtOAc (400 mL), and the mixture was washed with sat. NaHCO₃ (2×200 mL) and brine (200 mL), dried (MgSO₄), filtered, and concentrated. The residue was purified by silica gel chromatography (80 g cartridge) eluting with hexanes and acetone (0-40%). The product was diluted in DCM (100 mL), and stirred at 23° C. for 5 min. The solid was filtered, washed with hexanes, and dried under high vacuum to provide the title compound as a solid (1.00 g, 36%). ¹H NMR (500 MHz, DMSO) δ 13.24 (s, 1H), 7.74 (s, 1H), 7.76-7.64 (m, 2H), 7.36-7.28 (m, 2H), 4.47 (s, 3H). m/z (ES⁺), [M+H]⁺ 244.2. HPLC (A05) t_{R}=2.27 min.

### Step 4: 5-(1H-benzimidazol-2-yl)-1-methyl-pyrazol-3-amine

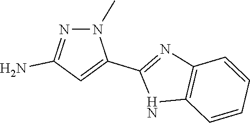 [SMILES: Cn1nc(N)cc1-c1nc2ccccc2[nH]1]

Zinc dust (1.34 g, 20.6 mmol) was added in portions at 0° C. to a mixture of 2-(2-methyl-5-nitro-pyrazol-3-yl)-1H-benzimidazole (1.00 g, 4.11 mmol) and NH₄Cl (2.20 g, 41.1 mmol) in acetone (45 mL) and water (15 mL). After the addition was complete, the mixture was warmed at 23° C. and stirred for 2 h. The mixture was filtered through Celite and washed with acetone. The filtrate was concentrated. The residue was diluted with water (100 mL) and EtOAc (100 mL), and the bi-phasic mixture was filtered. The aq. phase was extracted with EtOAc (2×100 mL), and the combined organic phases were washed with brine (100 mL), dried (MgSO₄), filtered, and concentrated. The residue was diluted in hexanes (100 mL) and stirred for 10 min. The mixture was filtered, and the solid was dried under high vacuum to provide the title compound as a solid (0.540 g, 62%). ¹H NMR (500 MHz, DMSO) δ 12.72 (s, 1H), 7.68 (d, J=7.7 Hz, 1H), 7.51 (d, J=7.7 Hz, 1H), 7.33-7.12 (m, 2H), 6.15 (s, 1H), 4.81 (s, 2H), 4.10 (s, 3H). m/z (ES⁺), [M+H]⁺ 214.2. HPLC (A05) t_{R}=1.91 min.

### Preparation of Compounds J-12, J-13, and J-14.

Compounds J-12, J-13, and J-14, as depicted in TABLE 4 below, were synthesized via procedures analogous to the above detailed synthesis of Compound J-11.

TABLE 4

| Structure | Characterization | Cpd # |
|---|---|---|
| 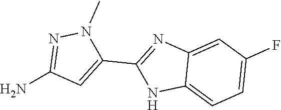 [SMILES: Cn1nc(N)cc1-c1nc2cc(F)ccc2[nH]1] | 5-(5-fluoro-1H-benzimidazol-2-yl)-1-methyl- pyrazol-3-amine ¹H NMR (400 MHz, DMSO) δ 12.86 (s, 1H), 7.72-7.48 (m, 1H), 7.48-7.27 (m, 1H), 7.08 (t, J = 9.3 Hz, 1H), 6.15 (s, 1H), 4.83 (s, 2H), 4.08 (s, 3H). m/z (ES⁺), [M + H]⁺ 232.0. | J-12 |
| 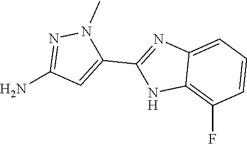 [SMILES: Cn1nc(N)cc1-c1nc2cccc(F)c2[nH]1] | 5-(7-fluoro-1H-benzimidazol-2-yl)-1-methyl- pyrazol-3-amine ¹H NMR (400 MHz, DMSO) δ 13.04 (s, 1H), 7.41-7.30 (m, 1H), 7.28-7.15 (m, 1H), 7.10-6.94 (m, 1H), 6.18 (s, 1H), 4.84 (s, 2H), 4.09 (s, 3H). m/z (ES⁺), [M + H]⁺ 232.0. | J-13 |
| 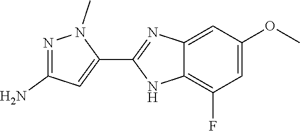 [SMILES: COc1cc(F)c2[nH]c(-c3cc(N)nn3C)nc2c1] | 5-(7-fluoro-5-methoxy-1H-benzimidazol-2-yl)-1- methyl-pyrazol-3-amine ¹H NMR (400 MHz, CD₃OD) δ 6.83 (s, 1H), 6.65 (d, J = 11.9 Hz, 1H), 6.14 (s, 1H), 4.06 (s, 3H), 3.84 (d, J = 2.4 Hz, 3H). m/z (ES⁺), [M + H]⁺ 262.3. | J-14 |

### EXAMPLE B3

### Preparation of Compound J-15

### Step 1: 2-methoxyethyl 1-(2-methoxyethyl)-5-nitro-pyrazole-3-carboxylate

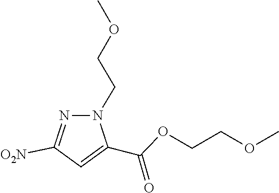 [SMILES: COCCOC(=O)c1cc([N+](=O)[O-])nn1CCOC]

2-Bromoethyl methyl ether (7.05 mL, 75.0 mmol) was added at 23° C. to a mixture of 5-nitro-1H-pyrazole-3-carboxylic acid (4.71 g, 30.0 mmol) and Cs₂CO₃ (29.3 g, 90.0 mmol) in DMF (300 mL). The mixture was heated at 60° C. for 18 h and filtered. The filtrate was concentrated. Water (100 mL) was added, and the aq. phase was extracted with EtOAc (3×100 mL). The combined organic phases were washed with brine (100 mL), dried (MgSO₄), filtered, and concentrated. The residue was purified by silica gel chromatography (330 g cartridge) eluting with hexanes and EtOAc (0-20%) to provide the title compound as an oil (3.47 g, 42%). ¹H NMR (500 MHz, CDCl₃) δ 7.43 (s, 1H), 4.86 (t, J=5.5 Hz, 2H), 4.50-4.46 (m, 2H), 3.80 (t, J=5.5 Hz, 2H), 3.71-3.68 (m, 2H), 3.42 (s, 3H), 3.31 (s, 3H). m/z (ES+), [M+H]⁺ 274.5. HPLC (A05) t_{R}=2.09 min. m/z (ES⁺), [M+H]⁺ 274.5. HPLC (A05) t_{R}=2.09 min.

### Step 2: 2-(2-methoxyethyl)-5-nitro-pyrazole-3-carboxylic acid

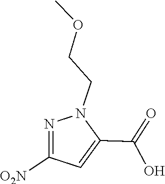

LiOH—H₂O (0.691 g, 16.5 mmol) was added to a solution of 2-methoxyethyl 2-(2-methoxyethyl)-5-nitro-pyrazole-3-carboxylate (1.50 g, 5.49 mmol) in MeOH (15 mL), THF (15 mL), and water (5 mL). The mixture was stirred at 23° C. for 3 h and concentrated. Water (50 mL) was added, and the aq. phase was acidified to pH 1 with conc. HCl. The mixture was filtered, and the solid was washed with water (10 mL) and dried under high vacuum to provide the title compound as a solid (0.863 g, 73%). ¹H NMR (500 MHz, DMSO) δ 14.24 (s, 1H), 7.48 (s, 1H), 4.80 (t, J=5.3 Hz, 2H), 3.75 (t, J=5.3 Hz, 2H), 3.22 (s, 3H). m/z (ES⁻), [M−H]⁻ 214.4. HPLC (A05) t_{R}=0.73 min.

### Step 3: 2-[2-(2-methoxyethyl)-5-nitro-pyrazol-3-yl]-1H-benzimidazole

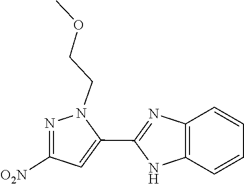 [SMILES: COCCn1nc([N+](=O)[O-])cc1-c1nc2ccccc2[nH]1]

DIEA (3.14 mL, 18.0 mmol) was added to a solution of HATU (2.16 g, 5.67 mmol) and 2-(2-methoxyethyl)-5-nitro-pyrazole-3-carboxylic acid (1.11 g, 5.15 mmol) in DMF (15 mL) at 0° C. The mixture was stirred at 0° C. for 15 min. o-Phenylenediamine (1.67 g, 15.5 mmol) was added. The mixture was warmed to 23° C. and stirred for 70 h. Water (150 mL) was added, and the aq. phase was extracted with EtOAc (6×20 mL). The combined organic phases were washed with sat. NaHCO₃ (50 mL) and brine (50 mL), dried (MgSO₄), filtered, and concentrated. The residue (m/z (ES⁺), [M−H]⁻ 304.5. HPLC (A05) t_{R}=2.06 min) was dissolved in AcOH (15 mL) and stirred at 70° C. for 1 h. The mixture was neutralized to pH 7 with 2.67 M NaOH, and the aqueous phase was extracted with EtOAc (6×20 mL). The combined organic phases were washed with sat. NaHCO₃ (50 mL) and brine (50 mL), dried (MgSO₄), filtered, and concentrated. The residue was purified by silica gel chromatography (40 g cartridge) eluting with hexanes and EtOAc (0-40%) to provide the title compound as a solid (0.980 g, 66%). ¹H NMR (400 MHz, CDCl₃) δ 7.86-7.83 (m, 1H), 7.53-7.49 (m, 1H), 7.41 (s, 1H), 7.38-7.31 (m, 2H), 4.70-4.65 (m, 2H), 4.08-4.04 (m, 2H), 3.52 (s, 3H). m/z (ES⁺), [M−H]⁻ 286.8. HPLC (A05) t_{R}=2.23 min.

### Step 4: 5-(1H-benzimidazol-2-yl)-1-(2-methoxyethyl)pyrazol-3-amine

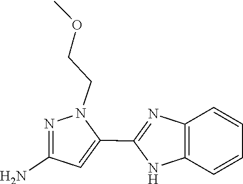 [SMILES: COCCn1nc(N)cc1-c1nc2ccccc2[nH]1]

Zinc dust (305 mg, 4.66 mmol) was added by portion-wise at 0° C. to a mixture of 2-[2-(2-methoxyethyl)-5-nitro-pyrazol-3-yl]-1H-benzimidazole (0.268 g, 0.933 mmol) and NH₄Cl (499 mg, 9.33 mmol) in acetone (5 mL) and water (5 mL). The mixture was warmed at 23° C. and stirred for 2 h. The mixture was filtered through Celite, washing with acetone. The filtrate was concentrated. Water (10 mL) was added, and the aq. phase was extracted with EtOAc (3×10 mL). The combined organic phases were washed with brine (10 mL), dried (MgSO₄), filtered, and concentrated to provide the title compound as a solid (0.230 g, 96%). ¹H NMR (400 MHz, DMSO) δ 12.72 (s, 1H), 7.66 (d, J=7.7 Hz, 1H), 7.50 (d, J=7.5 Hz, 1H), 7.27-7.16 (m, 2H), 6.14 (s, 1H), 4.86 (s, 2H), 4.78 (t, J=6.0 Hz, 2H), 3.69 (t, J=6.0 Hz, 2H), 3.18 (s, 3H). m/z (ES⁻). [M−H]⁻ 256.5. HPLC (A05) t_{R}=1.83 min.

### EXAMPLE B4

### Preparation of Compound J-16

### Step 1: allyl 2-allyl-5-nitro-pyrazole-3-carboxylate

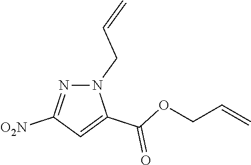 [SMILES: C=CCOC(=O)c1cc([N+](=O)[O-])nn1CC=C]

Allyl bromide (1.10 mL, 12.7 mmol) was added at 23° C. to a mixture of 5-nitro-1H-pyrazole-3-carboxylic acid (1.00 g, 6.37 mmol) and Na₂CO₃ (2.02 g, 19.1 mmol) in DMF (50 mL). The mixture was stirred at 23° C. for 72 h and filtered. The filtrate was concentrated, and water (50 mL) was added to the residue. The aq. phase was extracted with EtOAc (5×20 mL). The combined organic phases were washed with brine (50 mL), dried (MgSO₄), filtered, and concentrated. The residue was purified by silica gel chromatography (25 g cartridge) eluting with hexanes and EtOAc (0-5%) to provide the title compound as an oil (630 mg, 42%). ¹H NMR (500 MHz, CDCl₃) δ 7.44 (s, 1H), 6.06-5.95 (m, 2H), 5.39 (ddq, J=30.2, 10.4, 1.3 Hz, 2H), 5.31-5.22 (m, 4H), 4.84-4.81 (m, 2H). m/z (ES⁺), no ionization. HPLC (A05) t_{R}=2.44 min.

Note: The other isomer (allyl 1-allyl-5-nitro-pyrazole-3-carboxylate) was also isolated as an oil (327 mg, 22%). ¹H NMR (500 MHz, CDCl₃) δ 7.56 (s, 1H), 6.07-5.95 (m, 2H), 5.45-5.39 (m, 1H), 5.34-5.21 (m, 6H), 4.87 (dt, J=5.9, 1.3 Hz, 2H). m/z (ES⁺), no ionization. HPLC (A05) t_{R}=2.44 min.

### Step 2: 2-allyl-5-nitro-pyrazole-3-carboxylic acid

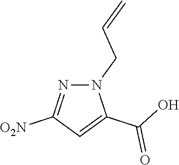

LiOH—H₂O) (334 mg, 7.97 mmol) was added to a solution of allyl 2-allyl-5-nitro-pyrazole-3-carboxylate (630 mg, 2.66 mmol) in MeOH (6 mL), THF (6 mL), and water (4 mL). The mixture was stirred at 23° C. for 2 h and concentrated. Water (50 mL) was added, and the aq. phase was acidified to pH 1 with conc. HCl. The aq. phase was extracted with EtOAc (3×20 mL). The combined organic phases were washed with brine (50 mL), dried (MgSO₄), filtered, and concentrated to provide the title compound as a solid (500 mg, 96%). ¹H NMR (400 MHz, DMSO) δ 14.22 (s, 1H), 7.50 (s, 1H), 6.09-5.98 (m, 1H), 5.24 (t, J=1.4 Hz, 2H), 5.22 (t, J=1.8 Hz, 2H). m/z (ES⁺), no ionization. HPLC (A05) t_{R}=1.65 min.

### Step 3: 2-(2-allyl-5-nitro-pyrazol-3-yl)-1H-benzimidazole

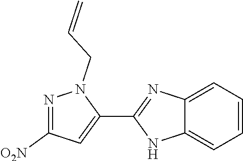 [SMILES: C=CCn1nc([N+](=O)[O-])cc1-c1nc2ccccc2[nH]1]

DIEA (1.21 mL, 6.92 mmol) was added to a solution of HATU (827 mg, 2.18 mmol) and 2-allyl-5-nitro-pyrazole-3-carboxylic acid (0.390 g, 1.98 mmol) in DMF (10 mL) at 0° C. The mixture was stirred at 0° C. for 15 min. o-Phenylenediamine (0.642 g, 5.93 mmol) was added. The mixture was warmed to 23° C. and stirred for 6 h. Water (100 mL) was added, and the aq. phase was extracted with EtOAc (5×10 mL). The combined organic phases were washed with sat. NaHCO₃ (50 mL) and brine (50 mL), dried (MgSO₄), filtered, and concentrated under reduced pressure. The residue was dissolved in AcOH (10 mL) and stirred at 70° C. for 1 h. The mixture was neutralized to pH 7 with 2.67 M NaOH, and the aq. phase was extracted with EtOAc (6×20 mL). The combined organic phases were washed with sat. NaHCO₃ (50 mL) and brine (50 mL), dried (MgSO₄), filtered, and concentrated. The residue was purified by silica gel chromatography (40 g cartridge) eluting with hexanes and EtOAc (0-40%) to provide the title compound as a solid (0.400 g, 75%). ¹H NMR (400 MHz, DMSO) δ 13.27 (s, 1H), 7.78 (s, 1H), 7.69 (s, 2H), 7.34-7.28 (m, 2H), 6.13 (ddt, J=17.1, 10.3, 5.6 Hz, 1H), 5.63 (dt, J=5.6, 1.5 Hz, 2H), 5.19 (ddq, J=30.2, 17.1, 1.5 Hz, 2H). m/z (ES⁺), [M−H]⁻ 268.5. HPLC (A05) t_{R}=2.36 min

### Step 4: 1-allyl-5-(1H-benzimidazol-2-yl)pyrazol-3-amine

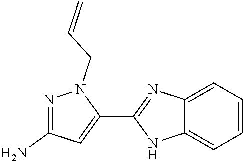 [SMILES: C=CCn1nc(N)cc1-c1nc2ccccc2[nH]1]

Zinc dust (455 mg, 6.96 mmol) was added to a mixture of 2-(2-allyl-5-nitro-pyrazol-3-yl)-1H-benzimidazole (375 mg, 1.39 mmol) and NH₄Cl (745 mg, 13.9 mmol) in acetone (3 mL) and water (3 mL). The mixture was stirred at 23° C. for 2 h. The mixture was filtered through Celite, and washed with acetone. The filtrate was concentrated. Water (30 mL) was added, and the aq. phase was extracted with EtOAc (3×20 mL). The combined organic phases were washed with brine (30 mL), dried (MgSO₄), filtered, and concentrated to provide the title compound as a solid (315 mg, 95%). ¹H NMR (400 MHz, DMSO) δ 12.74 (s, 1H), 7.66 (d, J=7.7 Hz, 1H), 7.51-7.47 (m, 1H), 7.26-7.16 (m, 2H), 6.19 (s, 1H), 6.03-5.93 (m, 1H), 5.26-5.22 (m, 2H), 5.02 (ddd, J=18.8, 13.7, 1.7 Hz, 2H), 4.87 (s, 2H). m/z (ES⁺), [M−H]⁻ 238.6. HPLC (A05) t_{R}=1.91 min.

### EXAMPLE B5

### Preparation of Compounds K-1 to K-14

### Representative Synthetic Scheme for the Syntheses of Compounds K-1 to K-14.

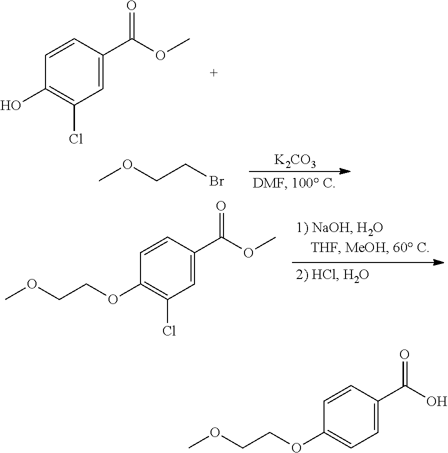

### Preparation of Compound K-1.

### Step 1: methyl 3-chloro-4-(2-methoxyethoxy)benzoate

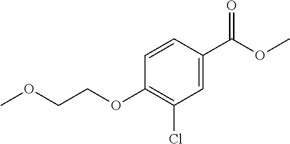

2-Bromoethyl methyl ether (0.121 mL, 1.29 mmol) was added to a mixture of methyl 3-chloro-4-hydroxy-benzoate (200 mg, 1.07 mmol) and K₂CO₃ (0.296 g, 2.14 mmol) in DMF (4 mL). The mixture was heated at 100° C. for 18 h. Water (50 mL) was added and the aq. phase was extracted with EtOAc (3×50 mL). The combined organic phases were washed with brine (30 mL), dried (MgSO₄), filtered, and concentrated. The residue was purified by silica gel chromatography (25 g cartridge) eluting with hexanes and EtOAc (0-20%) to provide the title compound as a solid (260 mg, 99%). ¹H NMR (400 MHz, DMSO) δ 7.93 (d, J=2.1 Hz, 1H), 7.90 (dd, J=8.6, 2.2 Hz, 1H), 7.29 (d, J=8.7 Hz, 1H), 4.30-4.27 (m, 2H), 3.83 (s, 3H), 3.73-3.70 (m, 2H), 3.33 (s, 3H). m/z (ES⁺), [M+H]⁺ No ionization. HPLC (A05) t_{R}=2.36 min.

### Step 2: 3-chloro-4-(2-methoxyethoxy)benzoic acid

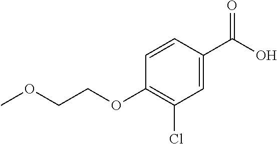

Methyl 3-chloro-4-(2-methoxyethoxy)benzoate (260 mg, 1.06 mmol) was dissolved in THF (1.50 mL) and MeOH (1.50 mL). 2.50 M aq. NaOH (1.49 mL, 3.72 mmol) was added. The mixture was heated at 60° C. for 1 h. The mixture was concentrated. Water (10 mL) was added, and the aq. phase was acidified to pH 2 with conc. aq. HCl. The mixture was filtered, and the solid was dried under high vacuum to provide the product as a solid (210 mg, 86%). ¹H NMR (500 MHz, DMSO) δ 12.95 (s, 1H), 7.90 (d, J=2.1 Hz, 1H), 7.87 (dd, J=8.6, 2.1 Hz, 1H), 7.26 (d, J=8.7 Hz, 1H), 4.27 (dd, J=5.4, 3.7 Hz, 2H), 3.73-3.69 (m, 2H), 3.34 (s, 3H). m/z (ES−), [M−H]⁻ 229.4. HPLC (A05) t_{R}=1.98 min.

### Preparation of Compound K-2.

### Step 1: ethyl 3-methoxy-4-(2-methoxyethoxy)benzoate

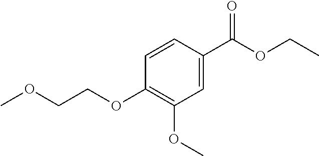 [SMILES: CCOC(=O)c1ccc(OCCOC)c(OC)c1]

2-Bromoethyl methyl ether (1.41 mL, 15.0 mmol) was added to a mixture of ethyl 4-hydroxy-3-methoxy-benzoate (1.96 g, 10.0 mmol) and K₂CO₃ (2.76 g, 20.0 mmol) in acetone (50 mL). The mixture was stirred at 55° C. for 18 h. After cooling to 23° C., the mixture was concentrated. Water (50 mL) was added, and the aq. phase was extracted with EtOAc (3×50 mL). The combined organic phases were washed with brine (50 mL), dried (MgSO₄), filtered, and concentrated. The residue was purified by silica gel chromatography (80 g cartridge) eluting with hexanes and EtOAc (0-30%) to provide the title compound as a solid (1.81 g, 71%). ¹H NMR (500 MHz, DMSO) δ 7.57 (dd, J=8.4, 2.0 Hz, 1H), 7.45 (d, J=2.0 Hz, 1H), 7.08 (d, J=8.5 Hz, 1H), 4.29 (q, J=7.1 Hz, 2H), 4.22-4.11 (m, 2H), 3.82 (s, 3H), 3.74-3.63 (m, 2H), 3.32 (s, 3H), 1.32 (t, J=7.1 Hz, 3H). m/z (ES⁺), [M+H]⁺ 255.3. HPLC (A05) t_{R}=2.25 min.

### Step 2: 3-methoxy-4-(2-methoxyethoxy)benzoic acid

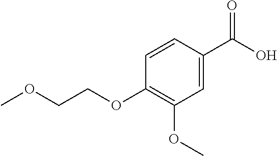 [SMILES: COCCOc1ccc(C(=O)O)cc1OC]

LiOH—H₂O (0.891 g, 21.2 mmol) was added to a mixture of ethyl 3-methoxy-4-(2-methoxyethoxy)benzoate (1.80 g, 7.08 mmol) in MeOH (20 mL), THF (20 mL), and water (6 mL). The mixture was stirred at 70° C. for 1 h and concentrated. Water (50 mL) was added, and the aq. phase was acidified to pH 3 with 1M aq. HCl. The mixture was filtered, and the solid was dried in a vacuum oven at 40° C. for 3 h to provide the title compound as a solid (1.05 g, 66%). ¹H NMR (500 MHz, DMSO) δ 12.68 (s, 1H), 7.54 (dd, J=8.4, 1.9 Hz, 1H), 7.45 (d, J=1.9 Hz, 1H), 7.06 (d, J=8.5 Hz, 1H), 4.29-4.09 (m, 2H), 3.81 (s, 3H), 3.76-3.60 (m, 2H), 3.32 (s, 3H). m/z (ES+), [M+H]⁺ 227.2. HPLC (A05) t_{R}=1.79 min.

### Preparation of Compound K-3.

### Step 1: methyl 4-(2-methoxyethoxy)-3-methyl-benzoate

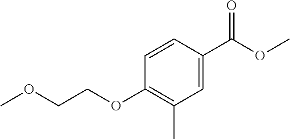

2-Bromoethyl methyl ether (1.41 mL, 15.0 mmol) was added to a mixture of methyl 4-hydroxy-3-methyl-benzoate (1.66 g, 10.0 mmol) and K₂CO₃ (2.76 g, 20.0 mmol) in acetone (50 mL). The mixture was stirred at 55° C. for 18 h. After cooling to 23° C., the mixture was concentrated. Water (50 mL) was added, and the aq. phase was extracted with EtOAc (3×50 mL). The combined organic phases were washed with brine (50 mL), dried (MgSO₄), filtered, and concentrated. The residue was purified by silica gel chromatography (80 g cartridge) with hexanes and EtOAc (0-30%) eluent to provide the title compound as an oil (0.589 g, 26%). ¹H NMR (500 MHz, DMSO) δ 7.80 (dd, J=8.5, 1.9 Hz, 1H), 7.78-7.75 (m, 1H), 7.05 (d, J=8.6 Hz, 1H), 4.23-4.14 (m, 2H), 3.81 (s, 3H), 3.75-3.67 (m, 2H), 3.34 (s, 3H), 2.20 (s, 3H). m/z (ES⁺), [M+H]⁺ 225.3. HPLC (A05) t_{R}=2.33 min.

### Step 2: 4-(2-methoxyethoxy)-3-methyl-benzoic acid

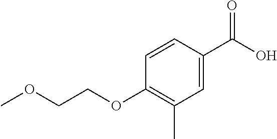

LiOH—H₂O (0.324 g, 7.72 mmol) was added to a mixture of methyl 4-(2-methoxyethoxy)-3-methyl-benzoate (0.577 g, 2.57 mmol) in MeOH (7 mL), THF (7 mL), and water (2 mL). The mixture was stirred at 70° C. for 1 h and concentrated. Water (50 mL) was added, and the aq. phase was acidified to pH 1 with conc. HCl. The mixture was filtered, and the solid was dried in a vacuum oven at 45° C. for 4 h to provide the title compound as a solid (0.474 g, 88%). ¹H NMR (400 MHz, DMSO) δ 12.52 (s, 1H), 7.77 (dd, J=8.5, 1.8 Hz, 1H), 7.75-7.72 (m, 1H), 7.03 (d, J=8.6 Hz, 1H), 4.22-4.14 (m, 2H), 3.75-3.64 (m, 2H), 3.34 (s, 3H), 2.19 (s, 3H). m/z (ES⁺), [M−H]⁻ 209.2. HPLC (A05) t_{R}=2.00 min.

### Preparation of Compound K-4.

### Step 1: methyl 3-fluoro-4-(2-methoxyethoxy)benzoate

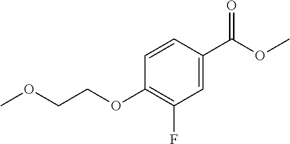

2-Bromoethyl methyl ether (0.580 mL, 6.17 mmol) was added to a solution of methyl 3-fluoro-4-hydroxy-benzoate (700 mg, 4.11 mmol) and Cs₂CO₃ (2.68 g, 8.23 mmol) in DMF (8 mL). The solution was heated at 100° C. for 2 h. EtOAc (150 mL) was added. The organic phase was washed with water (3×50 mL) and brine (50 mL), dried (MgSO₄), filtered, and concentrated to provide the title compound as a solid (930 mg, 99%). ¹H NMR (400 MHz, DMSO) δ 7.74-7.69 (m, 1H), 7.65 (dd, J=11.9, 2.1 Hz, 1H), 7.25 (t, J=8.6 Hz, 1H), 4.23-4.19 (m, 2H), 3.77 (s, 3H), 3.67-3.62 (m, 2H), 3.26 (s, 3H). m/z (ES⁺), No ionization. HPLC (A05) t_{R}=2.23 min.

### Step 2: 3-fluoro-4-(2-methoxyethoxy)benzoic acid

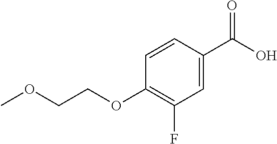

Methyl 3-fluoro-4-(2-methoxyethoxy)benzoate (930 mg, 4.08 mmol) was dissolved in THF (4 mL), MeOH (4 mL), and water (4 mL). LiOH—H₂O (0.513 g, 12.2 mmol) was added. The mixture was heated at 60° C. for 1 h. The mixture was partially concentrated, and water (10 mL) was added. The mixture was acidified to pH 2 with aq. HCl (1 M), at which point a precipitate formed. The mixture was filtered, and the solid was dried under high vacuum to provide the title compound as a solid (743 mg, 85%). ¹H NMR (400 MHz, CDCl₃) δ 7.86 (ddd, J=8.6, 2.0, 1.2 Hz, 1H), 7.80 (dd, J=11.5, 2.1 Hz, 1H), 7.03 (t, J=8.3 Hz, 1H), 4.29-4.25 (m, 2H), 3.83-3.79 (m, 2H), 3.47 (s, 3H). m/z (ES⁺), [M−H]⁻ 213.1. HPLC (A05) t_{R}=1.85 min.

### Preparation of Compound K-5.

### Step 1: methyl 4-(2-methoxyethoxy)-3-(trifluoromethyl)benzoate

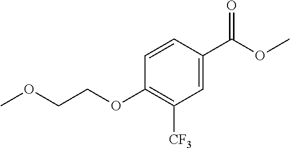

2-Bromoethyl methyl ether (0.448 mL, 4.77 mmol) was added to a solution of methyl 4-hydroxy-3-(trifluoromethyl)benzoate (700 mg, 3.18 mmol) and Cs₂CO₃ (2.07 g, 6.36 mmol) in DMF (8 mL). The solution was heated at 100° C. for 2 h. EtOAc (150 mL) was added. The organic phase was washed with water (3×50 mL) and brine (50 mL), dried (MgSO₄), filtered, and concentrated to provide the title compound as a solid (882 mg, 99%). ¹H NMR (400 MHz, CDCl₃) δ 8.27 (d, J=2.1 Hz, 1H), 8.18 (dd, J=8.7, 2.2 Hz, 1H), 7.05 (d, J=8.7 Hz, 1H), 4.28-4.25 (m, 2H), 3.91 (s, 3H), 3.83-3.79 (m, 2H), 3.46 (s, 3H). m/z (ES⁺), No ionization. HPLC (A05) t_{R}=2.45 min.

### Step 2: 4-(2-methoxyethoxy)-3-(trifluoromethyl)benzoic acid

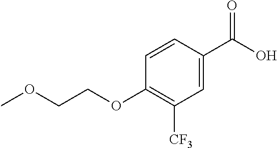 [SMILES: COCCOc1ccc(C(=O)O)cc1C(F)(F)F]

Methyl 4-(2-methoxyethoxy)-3-(trifluoromethyl)benzoate (882 mg, 3.17 mmol) was dissolved in THF (4 mL), MeOH (4 mL), and water (4 mL). LiOH—H₂O (0.399 g, 9.51 mmol) was added. The mixture was heated at 60° C. for 1 h. The mixture was partially concentrated, and water (10 mL) was added. The mixture was acidified to pH 2 with aq. HCl (1 M), at which point a precipitate formed. The mixture was filtered, and the solid was dried under high vacuum to provide the title compound as a solid (688 mg, 82%). ¹H NMR (400 MHz, CDCl₃) δ 8.34 (d, J=2.1 Hz, 1H), 8.24 (dd, J=8.7, 2.2 Hz, 1H), 7.09 (d, J=8.8 Hz, 1H), 4.32-4.28 (m, 2H), 3.83 (dd, J=5.3, 4.3 Hz, 2H), 3.47 (s, 3H). m/z (ES⁺), [M−H]⁻ 263.4. HPLC (A05) t_{R}=2.09 min.

### Preparation of Compound K-6.

### Step 1: methyl 3-cyano-4-(2-methoxyethoxy)benzoate

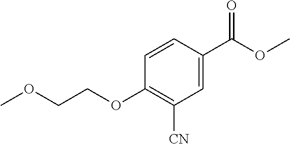

2-Bromoethyl methyl ether (0.358 mL, 3.81 mmol) was added to a solution of methyl 3-cyano-4-hydroxy-benzoate (450 mg, 2.54 mmol) and Cs₂CO₃ (1.66 g, 5.08 mmol) in DMF (6 mL). The solution was heated at 75° C. for 18 h. EtOAc (80 mL) was added. The organic phase was washed with water (3×60 mL) and brine (50 mL), dried (Na₂SO₄), filtered, and concentrated. The residue was dried in a vacuum oven at 50° C. for 18 h to provide the title compound as a solid (577 mg, 97%). ¹H NMR (400 MHz, DMSO) δ 8.23 (dd, J=3.4, 2.1 Hz, 1H), 8.19 (ddd, J=8.9, 2.2, 1.4 Hz, 1H), 7.40 (dd, J=9.0, 3.7 Hz, 1H), 4.39-4.35 (m, 2H), 3.85 (s, 3H), 3.74-3.71 (m, 2H), 3.34 (s, 3H). m/z (ES⁺), No ionization. HPLC (A05) t_{R}=2.16 min.

### Step 2: 3-cyano-4-(2-methoxyethoxy)benzoic acid

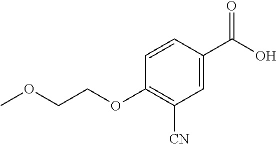 [SMILES: COCCOc1ccc(C(=O)O)cc1C#N]

Methyl 3-cyano-4-(2-methoxyethoxy)benzoate (577 mg, 2.45 mmol) was dissolved in THF (3 mL), MeOH (3 mL), and water (3 mL). LiOH—H₂O (0.206 g, 4.91 mmol) was added. The mixture was stirred for 30 min at 23° C. The mixture was partially concentrated, and water (10 mL) was added. The mixture was acidified to pH 2 with aq. HCl (1 M), at which point a precipitate formed. The mixture was filtered, and the solid was dried under vacuum to provide the title compound as a solid (436 mg, 80%). ¹H NMR (400 MHz, DMSO) δ 13.17 (s, 1H), 8.18 (dt, J=8.7, 2.0 Hz, 2H), 7.37 (d, J=8.8 Hz, 1H), 4.39-4.33 (m, 2H), 3.75-3.69 (m, 2H), 3.34 (s, 3H). m/z (ES⁺), [M−H]⁻ 220.4. HPLC (A05) t_{R}=1.76 min.

### Preparation of Compound K-7.

### Step 1: methyl 3-chloro-4-(3-methoxypropoxy)benzoate

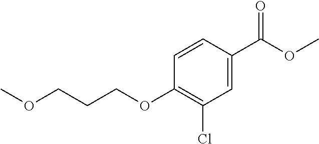

1-Bromo-3-methoxy-propane (0.331 mL, 2.94 mmol) was added to a solution of methyl 3-chloro-4-hydroxy-benzoate (300 mg, 1.61 mmol) and Cs₂CO₃ (1.05 g, 3.22 mmol) in DMF (6 mL). The solution was heated at 100° C. for 4 h. Water (50 mL) was added, and the aq. phase was extracted with EtOAc (3×50 mL). The combined organic phases were washed with water (50 mL) and brine (30 mL), dried (MgSO₄), filtered, and concentrated to provide the title compound as a solid (403 mg, 97%). ¹H NMR (400 MHz, DMSO) δ 7.93 (d, J=2.1 Hz, 1H), 7.90 (dd, J=8.6, 2.2 Hz, 1H), 7.28 (d, J=8.6 Hz, 1H), 4.20 (t, J=6.3 Hz, 2H), 3.83 (s, 3H), 3.50 (t, J=6.2 Hz, 2H), 3.25 (s, 3H), 2.04-1.96 (m, 2H). HPLC (A05) t_{R}=2.49 min.

### Step 2: 3-chloro-4-(3-methoxypropoxy)benzoic acid

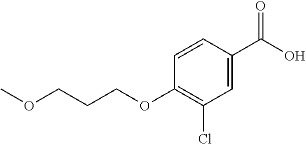 [SMILES: COCCCOc1ccc(C(=O)O)cc1Cl]

Methyl 3-chloro-4-(3-methoxypropoxy)benzoate (400 mg, 1.55 mmol) was dissolved in THF (2.86 mL) and MeOH (2.86 mL). 2.50 M aq. NaOH (1.86 mL, 4.64 mmol) was added. The mixture was heated at 60° C. for 1 h. The mixture was partially concentrated, and water (10 mL) was added. The mixture was acidified to pH 2 with aq. HCl (1 M), at which point a precipitate formed. The mixture was filtered, and the solid was dried under high vacuum to provide the title compound as a solid (319 mg, 84%). ¹H NMR (400 MHz, DMSO) δ 7.90 (d, J=2.1 Hz, 1H), 7.87 (dd, J=8.6, 2.1 Hz, 1H), 7.25 (d, J=8.7 Hz, 1H), 4.19 (t, J=6.3 Hz, 2H), 3.50 (t, J=6.3 Hz, 2H), 3.25 (s, 3H), 2.00 (p, J=6.3 Hz, 2H). m/z (ES), [M−H]⁻ 243.1. HPLC (A05) t_{R}=2.10 min.

### Preparation of Compound K-8.

### Step 1: methyl 4-[2-[tert-butyl(dimethyl)silyl]oxyethoxy]-3-chloro-benzoate

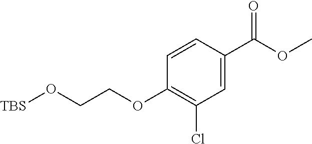

2-Bromoethoxy-tert-butyl-dimethyl-silane (1.15 mL, 5.36 mmol) was added to a solution of methyl 3-chloro-4-hydroxy-benzoate (1,000 mg, 5.36 mmol) and K₂CO₃ (1.11 g, 8.04 mmol) in DMF (15 mL). The solution was heated at 70° C. for 18 h. After cooling to 23° C., EtOAc (150 mL) was added, and the organic phase was washed with water (3×75 mL) and brine (75 mL), dried (Na₂SO₄), filtered, and concentrated to provide the title compound as a solid (1.84 g, 99%). ¹H NMR (400 MHz, DMSO) δ 7.93 (d, J=2.1 Hz, 1H), 7.89 (dd, J=8.7, 2.2 Hz, 1H), 7.29 (d, J=8.7 Hz, 1H), 4.26-4.21 (m, 2H), 3.98-3.95 (m, 2H), 3.83 (s, 3H), 0.85 (s, 9H), 0.06 (s, 6H). m/z (ES⁺), [M+H]^{P} no ionization. HPLC (A05) t_{R}=3.07 min.

### Step 2: 3-chloro-4-(2-hydroxyethoxy)benzoic acid

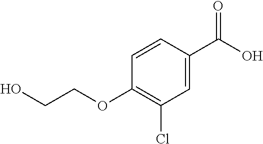 [SMILES: O=C(O)c1ccc(OCCO)c(Cl)c1]

Methyl 4[2-[tert-butyl(dimethyl)silyl]oxyethoxy]-3-chloro-benzoate (1.84 g, 5.33 mmol) was dissolved in THF (7.50 mL), MeOH (7.50 mL), and water (7.50 mL). LiOH—H₂O (0.672 g, 16.0 mmol) was added. The mixture was stirred at 23° C. for 18 h. The mixture was partially concentrated, and water (20 mL) was added. The mixture was acidified to pH 2 with aq. HCl (1 M), at which point a precipitate formed. The mixture was filtered, and the solid was dried under high vacuum to provide the title compound as a solid (1.04 g, 90%). ¹H NMR (400 MHz, DMSO) δ 12.94 (s, 1H), 7.90 (d, J=2.1 Hz, 1H), 7.87 (dd, J=8.6, 2.1 Hz, 1H), 7.26 (d, J=8.7 Hz, 1H), 4.92 (t, J=5.4 Hz, 1H), 4.20-4.14 (m, 2H), 3.76 (q, J=5.1 Hz, 2H). m/z (ES), [M−H]⁻ 215.4. HPLC (A05) t_{R}=1.69 min.

### Preparation of Compound K-9.

### Steps 1 and 2: 4-(2-hydroxyethoxy)-3-methoxy-benzoic acid

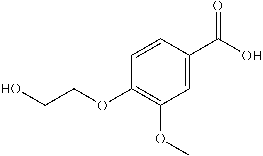

2-Bromoethanol (0.710 mL, 10.0 mmol) was added to a mixture of ethyl 4-hydroxy-3-methoxy-benzoate (981 mg, 5.00 mmol) and Cs₂CO₃ (3.26 g, 10.0 mmol) in DMF (11 mL). The mixture was stirred at 50° C. for 16 h. Additional 2-bromoethanol (0.710 mL, 10.0 mmol) was added. The mixture was stirred at 50° C. for 2 h and diluted with water (100 mL). The aq. phase was extracted with EtOAc (2×100 mL), and the combined organic phases were dried (Na₂SO₄), filtered, and concentrated. The product was purified by silica gel chromatography (40 g cartridge) eluting with hexanes and EtOAc (0-80%). The residue (m/z: ES⁺ [M+H]⁺ 240.5. LCMS (A05) t_{R}=1.99 min) was dissolved in MeOH (15 mL), THF (15 mL), and water (5 mL). LiOH—H₂O (0.629 g, 15.0 mmol) was added. The mixture was heated at 70° C. for 30 min and concentrated. The residue was diluted with water (50 mL), and the aq. phase was acidified to pH 2 with 1M aq. HCl. The mixture was filtered, and the solid was dried under high vacuum to provide the title compound as a solid (349 mg, 32% over two steps). ¹H NMR (400 MHz, DMSO) δ 12.59 (s, 1H), 7.48 (d, J=8.1 Hz, 1H), 7.39 (s, 1H), 6.99 (d, J=8.1 Hz, 1H), 4.84 (s, 1H), 3.99 (s, 2H), 3.75 (s, 3H), 3.68 (s, 2H). m/z: ES″ [M−H]⁻ 211.4. HPLC (A05) t_{R}=1.54 min.

### Preparation of Compound K-10.

### Step 1: methyl 4-(2-hydroxyethoxy)-3-methyl-benzoate

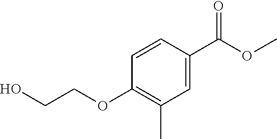

2-Bromoethanol (0.710 mL, 10.0 mmol) was added to a solution of methyl 4-hydroxy-3-methyl-benzoate (831 mg, 5.00 mmol) and Cs₂CO₃ (3.26 g, 10.0 mmol) in DMF (10 mL). The solution was heated at 50° C. for 4 h. Additional 2-bromoethanol (0.710 mL, 10.0 mmol) was added, and the mixture was stirred at 50° C. for 18 h. EtOAc (100 mL) was added, and the organic phase was washed with water (2×100 mL) and brine (100 mL), dried (MgSO₄), filtered, and concentrated. The residue was purified by silica gel chromatography (40 g cartridge) eluting with hexanes and EtOAc (0-60%) to provide the title compound as a solid (0.621 g, 59%). ¹H NMR (400 MHz, DMSO) δ 7.82-7.78 (m, 1H), 7.77-7.74 (m, 1H), 7.05 (d, J=8.6 Hz, 1H), 4.89 (t, J=5.5 Hz, 1H), 4.08 (t, J=5.0 Hz, 2H), 3.81 (s, 3H), 3.79-3.72 (m, 2H), 2.21 (s, 3H). m/z (ES⁺), [M+H]⁺ 211.5. HPLC (A05) t_{R}=2.02 min.

### Step 2: 4-(2-hydroxyethoxy)-3-methyl-benzoic acid

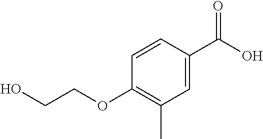

LiOH—H₂O (0.369 g, 8.79 mmol) was added to a mixture of methyl 4-(2-hydroxyethoxy)-3-methyl-benzoate (0.616 g, 2.93 mmol) in MeOH (8 mL), THF (8 mL), and water (2.50 mL). The mixture was stirred at 70° C. for 1.5 h and concentrated. Water (25 mL) was added, and the aq. phase was acidified to pH 2 with 1M aq. HCl. The mixture was filtered, and the solid was dried in a vacuum oven at 45° C. for 4 h to provide the title compound as a solid (0.408 g, 71%). ¹H NMR (500 MHz, DMSO) δ 12.50 (s, 1H), 7.77 (dd, J=8.5, 2.0 Hz, 1H), 7.73 (d, J=1.4 Hz, 1H), 7.02 (d, J=8.6 Hz, 1H), 4.87 (s, 1H), 4.08 (t, J=5.0 Hz, 2H), 3.76 (s, 2H), 2.20 (s, 3H). m/z (ES⁺), [M−H]⁻ 195.4. HPLC (A05) t_{R}=1.72 min.

### Preparation of Compound K-11.

### Step 1: methyl 3-fluoro-4-(2-hydroxyethoxy)benzoate

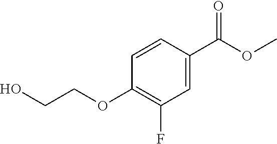

2-Bromoethanol (1.67 mL, 23.50 mmol) was added to a solution of methyl 3-fluoro-4-hydroxy-benzoate (1.00 g, 5.88 mmol) and Cs₂CO₃ (3.83 g, 11.8 mmol) in DMF (11.8 mL). The solution was heated at 50° C. for 4 h. Additional 2-bromoethanol (0.84 mL, 11.6 mmol) was added, and the mixture was stirred at 50° C. for 18 h. EtOAc (100 mL) was added, and the organic phase was washed with water (2×100 mL), brine (100 mL), dried (MgSO₄), filtered, and concentrated. The residue was purified by silica gel chromatography (40 g cartridge) eluting with hexanes and EtOAc (0-60%) to provide the title compound as a solid (0.621 g, 59%). ¹H NMR (400 MHz, CDCl₃) δ 7.84-7.70 (m, 2H), 7.00 (t, J=8.3 Hz, 1H), 4.23-4.19 (m, 4.0 Hz, 2H), 4.02 (m, 2H), 3.90 (s, 3H), 2.11 (t, J=6.0 Hz, 1H). m/z (ES⁺), [M+H]⁺=215.2. HPLC (A05) t_{R}=2.09 min.

### Step 2: 3-fluoro-4-(2-hydroxyethoxy)benzoic acid

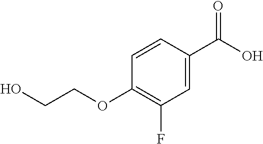 [SMILES: O=C(O)c1ccc(OCCO)c(F)c1]

LiOH—H₂O (0.74 g, 17.60 mmol) was added to a mixture of methyl 3-fluoro-4-(2-hydroxyethoxy)benzoate (1.26 g, 5.88 mmol) in MeOH (16.40 mL), THF (16.40 mL), and water (5.11 mL). The mixture was stirred at 70° C. for 1.5 h and concentrated. Water (40 mL) was added, and the aq. phase was acidified to pH 2 with 1M aq. HCl. The mixture was filtered, and the solid was dried in a vacuum oven at 45° C. for 4 h to provide the title compound as a solid (0.690 g, 59%). ¹H NMR (400 MHz, DMSO) δ 12.93 (s, 1H), 7.74 (ddd, J=8.6, 2.0, 1.1 Hz, 1H), 7.66 (dd, J=11.9, 2.1 Hz, 1H), 7.27 (t, J=8.6 Hz, 1H), 4.95 (s, 1H), 4.15 (t, J=4.2 Hz, 2H), 3.75 (d, J=4.3 Hz, 2H). m/z (ES⁻), [M−H]⁻ 199.4. HPLC (A05) t_{R}=1.51 min.

### Preparation of Compound K-12.

### Step 1: methyl 4-[2-[tert-butyl(dimethyl)silyl]oxyethoxy]-3-(trifluoromethyl)-benzoate

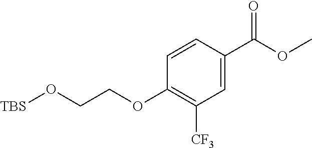

2-Bromoethoxy-tert-butyl-dimethyl-silane (1.03 mL, 4.77 mmol) was added to a solution of methyl 4-hydroxy-3-(trifluoromethyl)benzoate (700 mg, 3.18 mmol) and Cs₂CO₃ (2.07 g, 6.36 mmol) in DMF (9 mL). The solution was heated at 75° C. for 18 h. EtOAc (150 mL) was added, and the organic phase was washed with water (3×60 mL) and brine (50 mL), dried (Na₂SO₄), filtered, and concentrated. The residue was purified by silica gel chromatography (25 g cartridge) eluting with hexanes and EtOAc (0-30%) to provide the title compound as a solid (1.06 g, 88%). ¹H NMR (500 MHz, DMSO) δ 8.18 (dd, J=8.8, 2.2 Hz, 1H), 8.10 (d, J=2.1 Hz, 1H), 7.42 (d, J=8.9 Hz, 1H), 4.31-4.27 (m, 2H), 3.97-3.92 (m, 2H), 3.85 (s, 3H), 0.84 (s, 9H), 0.03 (s, 6H). HPLC (A05) t_{R}=3.07 min.

### Step 2: 4-(2-methoxyethoxy)-3-(trifluoromethyl)benzoic acid

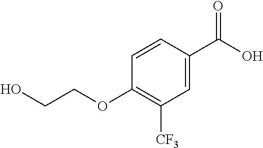 [SMILES: O=C(O)c1ccc(OCCO)c(C(F)(F)F)c1]

Methyl 4-[2-[tert-butyl(dimethyl)silyl]oxyethoxy]-3-(trifluoromethyl)benzoate (1.06 g, 2.80 mmol) was dissolved in THF (4 mL), MeOH (4 mL), and water (4 mL). LiOH—H₂O (0.353 g, 8.40 mmol) was added. The mixture was stirred at 23° C. for 18 h. The mixture was partially concentrated, and water (20 mL) was added. The mixture was acidified to pH 2 with aq. HCl (1 M), at which point a precipitate formed. The mixture was filtered, and the solid was dried under high vacuum to provide the title compound as a solid (585 mg, 84%). ¹H NMR (400 MHz, DMSO) δ 13.09 (s, 1H), 8.16 (dd, J=8.7, 2.2 Hz, 1H), 8.09 (d, J=2.1 Hz, 1H), 7.39 (d, J=8.8 Hz, 1H), 4.89 (t, J=5.2 Hz, 1H), 4.22 (t, J=5.0 Hz, 2H), 3.75 (q, J=5.1 Hz, 2H). ¹⁹F NMR (376 MHz, DMSO) δ −61.42 (s). m/z (ES⁺), [M−H]⁻ 249.4. HPLC (A05) t_{R}=1.82 min.

### Preparation of Compound K-13.

### Step 1: methyl 4-[2-[tert-butyl(dimethyl)silyl]oxyethoxy]-3-cyano-benzoate

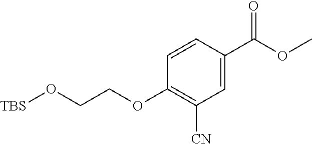

2-Bromoethoxy-tert-butyl-dimethyl-silane (0.912 mL, 4.23 mmol) was added to a solution of methyl 3-cyano-4-hydroxy-benzoate (500 mg, 2.82 mmol) and Cs₂CO₃ (1.84 g, 5.64 mmol) in DMF (8 mL). The solution was heated at 50° C. for 18 h. EtOAc (150 mL) was added, and the organic phase was washed with water (3×60 mL) and brine (50 mL), dried (Na₂SO₄), filtered, and concentrated. The residue was purified by silica gel chromatography (25 g cartridge) with hexanes and EtOAc (0-20%) eluent to provide the title compound as a solid (622 mg, 66%). ¹H NMR (400 MHz, DMSO) δ 8.23 (d, J=2.2 Hz, 1H), 8.18 (dd, J=8.9, 2.2 Hz, 1H), 7.41 (d, J=9.0 Hz, 1H), 4.35-4.30 (m, 2H), 3.99-3.94 (m, 2H), 3.85 (s, 3H), 0.84 (s, 9H), 0.06 (s, 6H). HPLC (A05) t_{R}=2.86 min.

### Step 2: 3-cyano-4-(2-hydroxyethoxy)benzoic acid

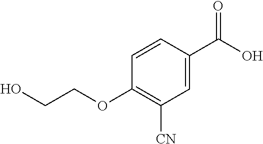 [SMILES: N#Cc1cc(C(=O)O)ccc1OCCO]

Methyl 4[2-[tert-butyl(dimethyl)silyl]oxyethoxy]-3-cyano-benzoate (622 mg, 1.85 mmol) was dissolved in THF (2.50 mL), MeOH (2.50 mL), and water (2.50 mL). LiOH—H₂O (0.233 g, 5.56 mmol) was added. The mixture was stirred at 23° C. for 30 min. The mixture was partially concentrated. EtOAc (100 mL) was added, and the organic phase was washed with aq. 1M HCl (50 mL) and brine (50 mL), dried (Na₂SO₄), filtered, and concentrated to provide the title as a solid (265 mg, 69%). ¹H NMR (400 MHz, DMSO) δ 12.14 (s, 1H), 8.18 (d, J=1.8 Hz, 1H), 8.16 (dd, J=8.8, 2.2 Hz, 1H), 7.38 (d, J=8.8 Hz, 1H), 4.98 (t, J=5.3 Hz, 1H), 4.28-4.23(m, 2H), 3.77 (dd, J=9.7, 5.1 Hz, 2H). m/z (ES⁺), [M−H]⁻ 206.4. HPLC (A05) t_{R}=1.48 min.

### Preparation of Compound K-14.

### Step 1: methyl 3-chloro-4-(2-morpholinoethoxy)benzoate

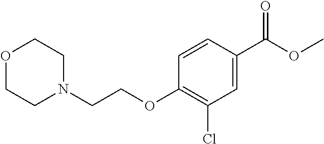 [SMILES: COC(=O)c1ccc(OCCN2CCOCC2)c(Cl)c1]

4-(2-Chloroethyl)morpholine hydrochloride (479 mg, 2.57 mmol) was added to a solution of methyl 3-chloro-4-hydroxy-benzoate (400 mg, 2.14 mmol) and K₂CO₃ (0.593 g, 4.29 mmol) in DMF (8 mL). The solution was heated at 100° C. for 18 h. Water (50 mL) was added, and the aq. phase was extracted with EtOAc (3×50 mL). The combined organic phases were washed with water (50 mL) and brine (30 mL), dried (MgSO₄), filtered, and concentrated to provide the title compound as a solid (411 mg, 64%). m/z (ES+), [M+H]⁺ 300.7. HPLC (A05) t_{R}=1.82 min.

### Step 2: 3-chloro-4-(2-morpholinoethoxy)benzoic acid hydrochloride

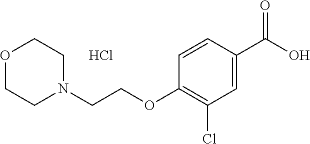 [SMILES: O=C(O)c1ccc(OCCN2CCOCC2)c(Cl)c1 ; Cl]

Methyl 3-chloro-4-(2-morpholinoethoxy)benzoate (411 mg, 1.37 mmol) was dissolved in THF (2.50 mL) and MeOH (2.50 mL). 2.50 M aq. NaOH (1.92 mL, 4.80 mmol) was added. The mixture was heated at 60° C. for 2 h. The mixture was partially concentrated. The aq. phase was washed with EtOAc (10 mL), and the organic phase was discarded. The aq. phase was acidified to pH 2 with conc. aq. HCl, at which point a precipitate formed. The mixture was filtered, and the solid was dried under high vacuum to provide the title compound as a solid (326 mg, 74%). ¹H NMR (400 MHz, DMSO) δ 11.62 (s, 1H), 10.87 (s, 1H), 7.91-7.86 (m, 2H), 7.27 (d, J=8.5 Hz, 1H), 4.61 (s, 2H), 3.97-3.86 (m, 4H), 3.34 (d, J=12.6 Hz, 2H), 3.17-2.98 (m, 4H). m/z (ES⁺), [M−Cl]⁺: 288.2. HPLC (A05) t_{R}=1.40 min.

### EXAMPLE B6

### Preparation of Compounds K-15 to K-18

Representative synthetic scheme for the syntheses of Compounds K-15 to K-18.

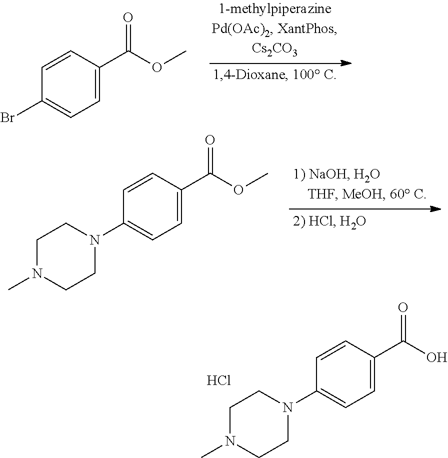

### Preparation of Compound K-15.

### Step 1: methyl 4-(4-methylpiperazin-1-yl)benzoate

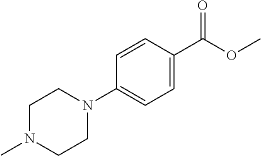

Nitrogen was bubbled through a mixture of methyl 4-bromobenzoate (25.0 g, 116 mmol), Cs₂CO₃ (75.8 g, 233 mmol), XantPhos (3.36 g, 5.81 mmol), Pd(OAc)₂ (0.653 g, 2.91 mmol), and 1-methylpiperazine (14.0 g, 140 mmol) in 1,4-dioxane (349 mL). The mixture was heated at 100° C. for 18 h. After cooling to 23° C., the mixture was filtered, and the filtrate was concentrated. The residue was dissolved in DCM (300 mL), and the mixture was filtered through a plug of silica, eluting with DCM (1.50 L, first filtrate) and DCM:MeOH (9:1, 1.80 L, second filtrate). The second filtrate was concentrated and diluted in hexanes (500 mL). The mixture was filtered, and the solid was dried under high vacuum to provide the title compound as a solid (18.1 g; 66%). ¹H NMR (400 MHz, DMSO) δ 7.81-7.75 (m, 2H), 7.00-6.95 (m, 2H), 3.77 (s, 3H), 3.31-3.28 (m, 4H), 2.45-2.39 (m, 4H), 2.21 (s, 3H). m/z (ES⁺), [M+H]⁺ 234.7. HPLC (A05) t_{R}=1.57 min.

### Step 2: 4-(4-methylpiperazin-1-yl)benzoic acid hydrochloride

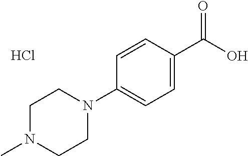 [SMILES: CN1CCN(c2ccc(C(=O)O)cc2)CC1 ; Cl]

Methyl 4-(4-methylpiperazin-1-yl)benzoate (18.1 g, 77.2 mmol) was dissolved in THF (110 mL) and MeOH (110 mL). 2.50 M aq. NaOH (108 mL, 270 mmol) was added. The mixture was heated at 60° C. for 2 h. The mixture was partially concentrated. The aq. phase was washed with EtOAc (300 mL) and acidified to pH 2 with conc. aq. HCl. The aq. phase was concentrated to half the volume and cooled to 23° C. The mixture was filtered, and the solid was set aside. The filtrate was concentrated to half the volume. The mixture was filtered, and the solid was combined with the first solid. The combined solids were washed with water and acetone and dried under high vacuum to provide the title compound as a solid (17.0 g, 86%). ¹H NMR (400 MHz, DMSO) δ 12.41 (s, 1H), 11.20 (s, 1H), 7.81 (d, J=9.0 Hz, 2H), 7.04 (d, J=9.1 Hz, 2H), 4.12-3.68 (m, 2H), 3.48-3.05 (m, 6H), 2.76 (s, 3H). m/z (ES⁺), [M−Cl]⁺: 222.2. HPLC (A05) t_{R}=0.37 min.

### Preparation of Compound K-16.

### Steps 1 and 2: 4-morpholinobenzoic acid

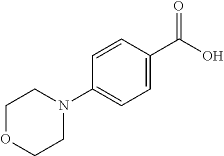 [SMILES: O=C(O)c1ccc(N2CCOCC2)cc1]

Nitrogen was bubbled through a solution of Pd(OAc)₂ (0.104 g, 0.465 mmol) and RuPhos (0.434 g, 0.930 mmol) in 1,4-dioxane (45 mL), and the mixture was heated at 70° C. for 10 min. The solution was added to a mixture of methyl 4-bromobenzoate (2.00 g, 9.30 mmol), morpholine (0.805 mL, 9.30 mmol), and Cs₂CO₃ (6.06 g, 18.6 mmol). The mixture was heated at 100° C. for 18 h. After cooling to 23° C., the mixture was filtered through Celite. The filtrate was concentrated, and the residue was purified by silica gel chromatography (40 g cartridge) eluting with hexanes and EtOAc (0-100%). The purified residue (m/z (ES⁺), [M+H]⁺ 222.6. HPLC (A05) t_{R}=2.16 min) was dissolved in THF (10 mL) and MeOH (10 mL). 2.50 M aq. LiOH—H₂O (11.2 mL, 27.9 mmol) was added. The mixture was heated at 60° C. for 1 h. The mixture was partially concentrated. Water (50 mL) was added, and the mixture was acidified to pH 2 with aq. HCl (1 M), at which point a precipitate formed. The mixture was filtered, and the solid was dried under high vacuum to provide the title compound as a solid (1.49 g, 77% over 2 steps). ¹H NMR (400 MHz, DMSO) δ 12.31 (s, 1H), 7.81-7.74 (m, 2H), 7.00-6.93 (m, 2H), 3.77-3.69 (m, 4H), 3.28-3.21 (m, 4H). m/z (ES⁺), [M+H]⁺ 208.1. HPLC (A05) t_{R}=1.82 min.

### Preparation of Compound K-17.

### Step 1: methyl 4-[(3R)-3-hydroxypyrrolidin-1-yl]benzoate

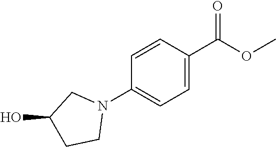 [SMILES: COC(=O)c1ccc(N2CC[C@@H](O)C2)cc1]

Nitrogen was bubbled through a mixture of Pd(OAc)₂ (20.9 mg, 0.0930 mmol) and Ruphos (86.8 mg, 0.186 mmol) in 1,4-dioxane (6 mL). The mixture was heated at 70° C. for 10 min. The solution was added to a mixture of methyl 4-bromobenzoate (400 mg, 1.86 mmol), (3R)-pyrrolidin-3-ol (0.392 mL, 4.84 mmol), and Cs₂CO₃ (1.21 g, 3.72 mmol). The mixture was heated at 120° C. for 18 h. After cooling to 23° C., the mixture was filtered through a pad of celite. The filtrate was concentrated, and the residue was purified by silica gel chromatography (40 g cartridge) eluting with hexanes and EtOAc (0-100%) to provide the title compound as a solid (332 mg; 81%). ¹H NMR (400 MHz, DMSO) δ 7.78-7.72 (m, 2H), 6.57-6.51 (m, 2H), 5.00 (s, 1H), 4.41 (s, 1H), 3.75 (s, 3H), 3.45 (dd, J=10.6, 4.7 Hz, 1H), 3.41-3.34 (m, 2H), 3.15 (d, J=10.8 Hz, 1H), 2.09-1.98 (m, 1H), 1.96-1.87 (m, 1H). m/z (ES⁺), [M+H]⁺ 222.6. HPLC (A05) t_{R}=1.99 min.

### Step 2: 4-[(3R)-3-hydroxypyrrolidin-1-yl]benzoic acid

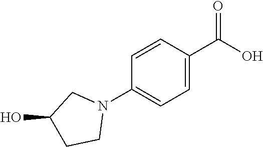 [SMILES: O=C(O)c1ccc(N2CC[C@@H](O)C2)cc1]

Methyl 4-[(3R)-3-hydroxypyrrolidin-1-yl]benzoate (275 mg, 1.24 mmol) was dissolved in THF (1.36 mL) and MeOH (1.36 mL). 2.50 M aq. NaOH (1.74 mL, 4.35 mmol) was added. The mixture was heated at 60° C. for 1.5 h. The mixture was concentrated. Water (15 mL) was added, and the aq. phase was acidified to pH 6 with aq. HCl (1 M), at which point a precipitate formed. The mixture was filtered, and the solid was dried under high vacuum to provide the title compound as a solid (125 mg, 48%). ¹H NMR (500 MHz, DMSO) δ 12.00 (s, 1H), 7.73 (d, J=8.8 Hz, 2H), 6.52 (d, J=8.8 Hz, 2H), 5.08-4.92 (m, 1H), 4.44-4.37 (m, 1H), 3.44 (dd, J=10.6, 4.7 Hz, 1H), 3.37 (ddd, J=11.9, 9.5, 3.1 Hz, 2H), 3.14 (d, J=10.5 Hz, 1H), 2.08-2.00 (m, 1H), 1.94-1.87 (m, 1H). m/z (ES), [M−H]⁻: 206.1. HPLC (A05) t_{R}=1.75 min.

### Preparation of Compound K-18.

### Step 1: methyl 4-[(3S)-3-hydroxypyrrolidin-1-yl]benzoate

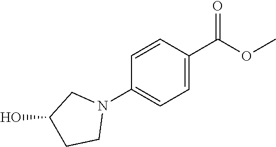 [SMILES: COC(=O)c1ccc(N2CC[C@H](O)C2)cc1]

Nitrogen was bubbled through a solution of Pd(OAc)₂ (20.9 mg, 0.0930 mmol) and Ruphos (86.8 mg, 0.186 mmol) in 1,4-dioxane (6 mL). The mixture was heated at 70° C. for 10 min. The solution was added to a mixture of methyl 4-bromobenzoate (400 mg, 1.86 mmol), (3S)-pyrrolidin-3-ol (0.392 mL, 4.84 mmol), and Cs₂CO₃ (1.21 g, 3.72 mmol). The mixture was heated at 120° C. for 18 h. After cooling to 23° C., the mixture was filtered through Celite. The filtrate was concentrated, and the residue was purified by silica gel chromatography (40 g cartridge) eluting with hexanes and EtOAc (0-100%) to provide the title compound as a solid (280 mg; 68%). ¹H NMR (400 MHz, DMSO) δ 7.73-7.69 (m, 2H), 6.52-6.48 (m, 2H), 4.96 (d, J=3.7 Hz, 1H), 4.40-4.34 (m, 1H), 3.71 (s, 3H), 3.40 (dd, J=10.7, 4.8 Hz, 1H), 3.37-3.29 (m, 2H), 3.11 (d, J=10.5 Hz, 1H), 2.04-1.95 (m, 1H), 1.91-1.83 (m, 1H). m/z (ES⁺), [M+H]⁺ 222.5. HPLC (A05) t_{R}=2.01 min.

### Step 2: 4-[(3S)-3-hydroxypyrrolidin-1-yl]benzoic acid

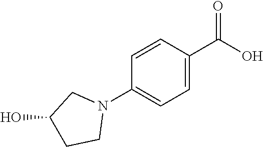 [SMILES: O=C(O)c1ccc(N2CC[C@H](O)C2)cc1]

Methyl 4-[(3S)-3-hydroxypyrrolidin-1-yl]benzoate (280 mg, 1.27 mmol) was dissolved in THF (2.10 mL) and MeOH (2.10 mL). 2.50 M aq. NaOH (1.52 mL, 3.80 mmol) was added. The mixture was heated at 60° C. and stirred for 3 h. The mixture was concentrated. Water (10 mL) was added, and the mixture was acidified to pH 6 with aq. HCl (1 M), at which point a precipitate formed. The mixture was filtered, and the solid was dried under high vacuum to provide the title compound as a solid (259 mg, 99%). ¹H NMR (500 MHz, DMSO) δ 12.01 (s, 1H), 7.74 (d, J=8.9 Hz, 2H), 6.52 (d, J=8.9 Hz, 2H), 5.00 (d, J=3.7 Hz, 1H), 4.41 (s, 1H), 3.44 (dd, J=10.6, 4.7 Hz, 1H), 3.41-3.35 (m, 2H), 3.15 (d, J=10.3 Hz, 1H), 2.08-1.99 (m, 1H), 1.95-1.87 (m, 1H). m/z (ES⁺), [M+H]⁺: 208.2. HPLC (A05) t_{R}=1.74 min.

### Preparation of Compound K-19.

### Step 1: methyl 3-chloro-4-(2-methoxyethylamino)benzoate

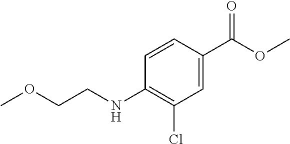

Nitrogen was bubbled through a solution of Pd(OAc)₂ (18 mg, 0.0802 mmol) and Xantphos (46.4 mg, 0.0802 mmol) in 1,4-dioxane (6 mL), and the mixture was heated at 70° C. for 10 min. Methyl 4-bromo-3-chloro-benzoate (400 mg, 1.60 mmol), 2-methoxyethanamine (0.279 mL, 3.21 mmol), and Cs₂CO₃ (1.04 g, 3.21 mmol) were added. The mixture was stirred at 120° C. for 18 h. After cooling to 23° C., the mixture was filtered through Celite. The filtrate was concentrated, and the residue was purified by silica gel chromatography (40 g cartridge) eluting with hexanes and EtOAc (0-40%) to provide the title compound as a solid (326 mg; 83%). ¹H NMR (400 MHz, DMSO) δ 7.77 (d, J=2.0 Hz, 1H), 7.72 (ddd, J=8.7, 2.0, 0.4 Hz, 1H), 6.83 (d, J=8.7 Hz, 1H), 6.05 (t, J=5.7 Hz, 1H), 3.77 (s, 3H), 3.51 (t, J=5.8 Hz, 2H), 3.40 (q, J=5.7 Hz, 2H), 3.28 (s, 3H). m/z (ES⁺), [M+H]⁺ 244.2. HPLC (A05) t_{R}=2.38 min.

### Step 2: 3-chloro-4-(2-methoxyethylamino)benzoic acid

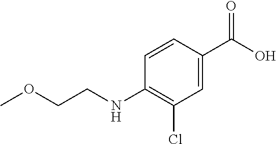

Methyl 3-chloro-4-(2-methoxyethylamino)benzoate (275 mg, 1.13 mmol) was dissolved in THF (1 mL) and MeOH (1 mL). 2.50 M aq. NaOH (1.58 mL, 3.95 mmol) was added. The mixture stirred at 60° C. for 20 min. The mixture was concentrated. Water (10 mL) was added, and the aq. phase was acidified to pH 6 with 1M aq. HCl drop-wise. The mixture was filtered, and the solid was dried in a vacuum oven at 40° C. for 18 h to provide the title compound as a solid (252 mg, 97%). ¹H NMR (400 MHz, DMSO) δ 12.42 (s, 1H), 7.74 (d, J=2.0 Hz, 1H), 7.70 (dd, J=8.4, 1.8 Hz, 1H), 6.81 (d, J=8.7 Hz, 1H), 5.95 (t, J=5.7 Hz, 1H), 3.52 (t, J=5.7 Hz, 2H), 3.39 (q, J=5.8 Hz, 2H), 3.28 (s, 3H). m/z (ES), [M−H]⁻: 228.1. HPLC (A05) t_{R}=2.03 min.

### Preparation of Compound K-20.

### Step 1: methyl 4-(4-hydroxy-1-piperidyl)benzoate

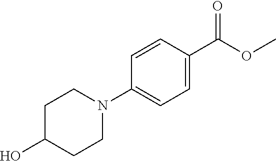 [SMILES: COC(=O)c1ccc(N2CCC(O)CC2)cc1]

Methyl 4-fluorobenzoate (1.68 mL, 13.0 mmol) and piperidin-4-ol (2.62 g, 26.0 mmol) were dissolved in MeCN (25 mL). K₂CO₃ (2.69 g, 19.5 mmol) was added, and the mixture was heated at 90° C. for 42 h. The mixture was filtered, and the filtrate was concentrated. The residue was purified by silica gel chromatography (40 g, cartridge) eluting with DCM and MeOH (0-5%) to provide the title compound (526 mg, 17%) as a solid. ¹H NMR (500 MHz, DMSO) δ 7.84-7.67 (m, 2H), 7.04-6.87 (m, 2H), 4.71 (d, J=4.3 Hz, 1H), 3.77 (s, 3H), 3.76-3.66 (m, 3H), 3.13-2.92 (m, 2H), 1.87-1.73 (m, 2H), 1.48-1.33 (m, 2H). m/z (ES⁺), [M+H]⁺ 236.2. HPLC (A05) t_{R}=2.00 min.

### Step 2: 4-(4-hydroxy-1-piperidyl)benzoic acid

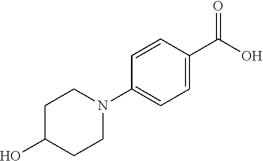 [SMILES: O=C(O)c1ccc(N2CCC(O)CC2)cc1]

LiOH—H₂O (0.140 g, 3.34 mmol) was added to a mixture of methyl 4-(4-hydroxy-1-piperidyl)benzoate (0.524 g, 2.23 mmol) in MeOH (2.25 mL), THF (2.25 mL), and water (0.400 mL). The mixture was heated at 70° C. for 5 h. After cooling to 23° C., the mixture was partially concentrated. Water (20 mL) was added, and the aq. phase was acidified to pH 6 with 1M aq. HCl dropwise. The mixture was filtered, and the solid was washed with water and dried under high vacuum to provide the title compound as a solid (454 mg, 92%). ¹H NMR (500 MHz, DMSO) δ 12.20 (br, 1H), 7.75 (d, J=8.9 Hz, 2H), 6.94 (d, J=9.0 Hz, 2H), 4.71 (br, 1H), 3.81-3.60 (m, 3H), 3.16-2.92 (m, 2H), 1.92-1.68 (m, 2H), 1.51-1.32 (m, 2H). m/z (ES⁺), [M+H]⁺ 222.2. HPLC (A05) t_{R}=1.69 min.

### EXAMPLE B7

### Preparation of Compounds L-1 to L-29

### Representative Synthetic Scheme for the Syntheses of Compounds L-1 to L-29.

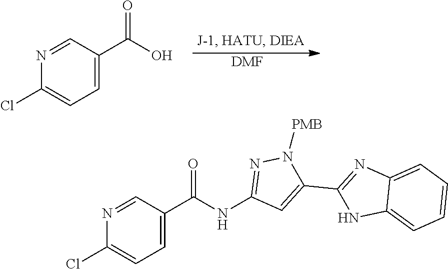

### Preparation of Compound L-1 (N-[5-(1H-benzimidazol-2-yl)-1-[(4-methoxyphenyl)methyl]pyrazol-3-yl]-6-chloro-pyridine-3-carboxamide)

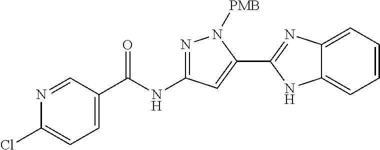

6-Chloropyridine-3-carboxylic acid (1.48 g, 9.39 mmol) and HATU (3.57 g, 9.39 mmol) were dissolved in DMF (60 mL) at 0° C. DIEA (3.22 mL, 18.8 mmol) was added, and the mixture was stirred at 0° C. for 5 min. 5-(1H-Benzimidazol-2-yl)-1-[(4-methoxyphenyl)methyl]pyrazol-3-amine (J-1) (3.00 g, 9.39 mmol) was added. The mixture was warmed to 23° C., stirred for 18 h, and poured into water (150 mL). The mixture was stirred at 23° C. for 5 min. The mixture was filtered, and the solid was washed with water (50 mL) and dried in a vacuum oven at 40° C. for 18 to provide the title compound as a solid (3.61 g, 84%). ¹H NMR (400 MHz, DMSO) δ 13.16 (s, 1H), 11.45 (s, 1H), 8.99 (d, J=2.4 Hz, 1H), 8.40 (dd, J=8.4, 2.5 Hz, 1H), 7.73 (d, J=8.5 Hz, 1H), 7.68 (d, J=8.3 Hz, 1H), 7.58 (s, 1H), 7.55 (d, J=7.3 Hz, 1H), 7.33-7.19 (m, 4H), 6.89-6.81 (m, 2H), 6.05 (s, 2H), 3.69 (s, 3H). m/z (ES⁺), [M+H]⁺ 459.4. HPLC (A05) t_{R}=2.60 min.

### Preparation of Compound L-2 (6-chloro-N-[5-(5-fluoro-1H-benzimidazol-2-yl)-1-[(4-methoxyphenyl)-methyl]pyrazol-3-yl]pyridine-3-carboxamide)

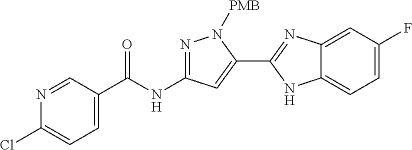

DIEA (0.914 mL, 5.25 mmol) was added to a solution of HATU (0.627 g, 1.65 mmol) and 6-chloropyridine-3-carboxylic acid (0.248 g, 1.57 mmol) in DMF (5 mL) at 0° C. The mixture was stirred at 0° C. for 15 min. 5-(5-Fluoro-1H-benzimidazol-2-yl)-1-[(4-methoxyphenyl)-methyl]pyrazol-3-amine (J-2) (0.506 g, 1.50 mmol) was added. The mixture was warmed to 23° C. and stirred for 18 h. Water (50 mL) was added, and the mixture was stirred at 23° C. for 5 min. The mixture was filtered, and the solid was purified by silica gel chromatography (40 g cartridge) eluting with hexanes and EtOAc (0-100%) to provide the title compound as a solid (0.458 g, 64%). ¹H NMR (500 MHz, DMSO) δ 13.31 (s, 1H), 11.46 (s, 1H), 9.00 (d, J=2.1 Hz, 1H), 8.40 (dd, J=8.4, 2.5 Hz, 1H), 7.74 [7.56] (br, 1H), 7.69 (d, J=8.3 Hz, 1H), 7.58 (s, 1H), 7.56 [7.35] (br, 1H), 7.29-7.20 (m, 2H), 7.12 [7.12] (br, 1H), 6.91-6.81 (m, 2H), 6.03 (s, 2H), 3.70 (s, 3H). m/z (ES⁺), [M+H]⁺ 477.4. HPLC (A05) t_{R}=2.54 min.

### Preparation of Compound L-3 (6-chloro-N-[5-(7-fluoro-1H-benzimidazol-2-yl)-1-[(4-methoxyphenyl)-methyl]pyrazol-3-yl]pyridine-3-carboxamide)

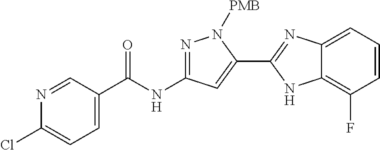

DIEA (0.914 mL, 5.25 mmol) was added to a solution of HATU (0.627 g, 1.65 mmol) and 6-chloropyridine-3-carboxylic acid (0.248 g, 1.57 mmol) in DMF (5 mL) at 0° C. The mixture was stirred at 0° C. for 15 min. 5-(7-Fluoro-1H-benzimidazol-2-yl)-1-[(4-methoxyphenyl)-methyl]pyrazol-3-amine (J-3) (0.506 g, 1.50 mmol) was added. The mixture was warmed to 23° C. and stirred for 18 h. Water (50 mL) was added, and the mixture was stirred at 23° C. for 5 min. The mixture was filtered, and the solid was purified by silica gel chromatography (40 g cartridge) eluting with hexanes and EtOAc (0-100%) to provide the title compound as a solid (0.426 g, 60%). ¹H NMR (500 MHz, DMSO) δ 13.48 (s, 1H), 11.48 (s, 1H), 9.01 (d, J=2.0 Hz, 1H), 8.41 (dd, J=8.4, 2.5 Hz, 1H), 7.71-7.66 (m, 1H), 7.62 (s, 1H), 7.40 (d, J=8.0 Hz, 1H), 7.31-7.21 (m, 3H), 7.11-7.03 (m, 1H), 6.90-6.82 (m, 2H), 6.04 (s, 2H), 3.70 (s, 3H). m/z (ES⁺), [M+H]⁺ 477.4. HPLC (A05) t_{R}=2.54 min.

### Preparation of Compound L-4 (6-chloro-N-[5-(5-chloro-1H-benzimidazol-2-yl)-1-[(4-methoxyphenyl)-methyl]pyrazol-3-yl]pyridine-3-carboxamide)

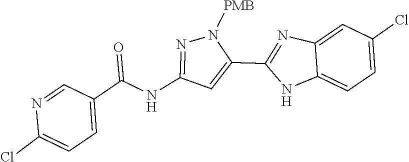

6-Chloropyridine-3-carboxylic acid (223 mg, 1.41 mmol) and HATU (0.537 g, 1.41 mmol) were dissolved in DMF (10 mL) at 0° C. DIEA (0.484 mL, 2.83 mmol) was added, and the mixture was stirred at 0° C. for 5 min. 5-(5-Chloro-1H-benzimidazol-2-yl)-1-[(4-methoxyphenyl)-methyl]pyrazol-3-amine (J-4) (500 mg, 1.41 mmol) was added. The mixture was warmed to 23° C. and stirred for 18 h. EtOAc (100 mL) was added. The organic phase was washed with water (3×50 mL) and brine (60 mL), dried (MgSO₄), filtered, and concentrated. The residue was purified by silica gel chromatography (25 g cartridge) eluting with hexanes and EtOAc (0-80%) to provide the title compound as a solid (437 mg, 63%). ¹H NMR (400 MHz, DMSO) δ 13.36 (s, 1H), 11.46 (s, 1H), 8.99 (d, J=2.5 Hz, 1H), 8.39 (dd, J=8.4, 2.5 Hz, 1H), 7.84-7.70 (m, 1H), 7.68 (dd, J=8.4, 0.6 Hz, 1H), 7.62-7.53 (m, 2H), 7.34-7.19 (m, 3H), 6.89-6.82 (m, 2H), 6.02 (s, 2H), 3.69 (s, 3H). m/z (ES⁺), [M+H]⁺ 494.4. HPLC (A05) t_{R}=2.64 min.

### Preparation of Compound L-5 (N-[5-(5-bromo-1H-benzimidazol-2-yl)-1-[(4-methoxyphenyl)methyl]pyrazol-3-yl]-6-chloro-pyridine-3-carboxamide)

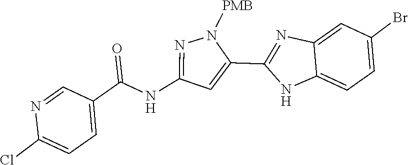

6-Chloropyridine-3-carboxylic acid (198 mg, 1.26 mmol) and HATU (0.477 g, 1.26 mmol) were dissolved in DMF (10 mL) at 0° C. DIEA (0.430 mL, 2.51 mmol) was added, and the mixture was stirred at 0° C. for 5 min. 5-(5-Bromo-1H-benzimidazol-2-yl)-1-[(4-methoxyphenyl)-methyl]pyrazol-3-amine (J-5) (500 mg, 1.26 mmol) was added. The mixture was stirred at 23° C. for 18 h. EtOAc (100 mL) was added. The organic phase was washed with water (3×50 mL) and brine (60 mL), dried (MgSO₄), filtered, and concentrated. The residue was purified by silica gel chromatography (25 g cartridge) eluting with hexanes and EtOAc (0-80%) to provide the title compound as a solid (446 mg, 66%). ¹H NMR (400 MHz, DMSO) δ 13.36 (s, 1H), 11.47 (s, 1H), 8.99 (d, J=2.5 Hz, 1H), 8.39 (dd, J=8.4, 2.5 Hz, 1H), 7.94 [7.52] (s, 1H), 7.74-7.65 (m, 2H), 7.59 (s, 1H), 7.44-7.35 (m, 1H), 7.24 (d, J=8.7 Hz, 2H), 6.89-6.83 (m, 2H), 6.01 (s, 2H), 3.69 (s, 3H). m/z (ES⁺), [M+H]⁺ 538.9. HPLC (A05) t_{R}=2.67 min.

### Preparation of Compound L-6 (6-chloro-N-[1-[(4-methoxyphenyl)methyl]-5-(5-methyl-1H-benzimidazol-2-yl)pyrazol-3-yl]pyridine-3-carboxamide)

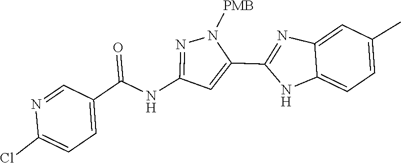

6-Chloropyridine-3-carboxylic acid (236 mg, 1.50 mmol) and HATU (0.570 g, 1.50 mmol) were dissolved in DMF (10 mL) at 0° C. DIEA (0.513 mL, 3.00 mmol) was added, and the mixture was stirred at 0° C. for 5 min. 1-[(4-Methoxyphenyl)methyl]-5-(5-methyl-1H-benzimidazol-2-yl)pyrazol-3-amine (J-6) (500 mg, 1.50 mmol) was added. The mixture was warmed to 23° C. and stirred for 18 h. EtOAc (100 mL) was added. The organic phase was washed with water (3×50 mL) and brine (60 mL), dried (MgSO₄), filtered, and concentrated. The residue was purified by silica gel chromatography (25 g cartridge) eluting with DCM and MeOH (0-10%) to provide the title compound as a solid in a 3:2 mixture of tautomers (511 mg, 72%). ¹H NMR (400 MHz, DMSO) δ 13.00 (d, J=14.4 Hz, 1H), 11.43 (s, 1H), 8.99 (dd, J=2.5, 0.6 Hz, 1H), 8.39 (dd, J=8.4, 2.5 Hz, 1H), 7.68 (dd, J=8.4, 0.6 Hz, 1H), 7.59 [7.42] (d, J=8.1 Hz, 1H), 7.54 (s, 1H), [7.52] 7.33 (s, 1H), 7.24 (d, J=8.4 Hz, 2H), [7.11] 7.06 (d, J=8.6 Hz, 1H), 6.88-6.83 (m, 2H), 6.03 (s, 2H), 3.69 (s, 3H), 2.45 [2.43] (s, 3H). m/z (ES⁺), [M+H]⁺ 473.4. HPLC (A05) t_{R}=2.57 min.

### Preparation of Compound L-7 (6-chloro-N-[1-[(4-methoxyphenyl)methyl]-5-[5-(trifluoromethyl)-1H-benzimidazol-2-yl]pyrazol-3-yl]pyridine-3-carboxamide)

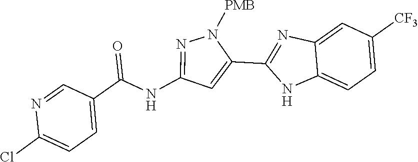

6-Chloropyridine-3-carboxylic acid (203 mg, 1.29 mmol) and HATU (0.491 g, 1.29 mmol) were dissolved in DMF (9 mL) at 0° C. HATU (0.491 g, 1.29 mmol) was added, and the mixture was stirred at 0° C. for 5 min. 1-[(4-Methoxyphenyl)methyl]-5-[5-(trifluoromethyl)-1H-benzimidazol-2-yl]pyrazol-3-amine (J-7) (500 mg, 1.29 mmol) was added. The mixture was warmed to 23° C. and stirred for 18 h. EtOAc (100 mL) was added. The organic phase was washed with water (3×50 mL) and brine (60 mL), dried (MgSO₄), filtered, and concentrated. The residue was purified by silica gel chromatography (25 g cartridge) eluting with DCM and MeOH (0-10%) to provide the title compound as a solid (498 mg, 73%). ¹H NMR (400 MHz, DMSO) δ [13.63] 13.61 (s, 1H), 11.49 (s, 1H), 9.00 (dd, J=2.5, 0.6 Hz, 1H), 8.40 (dd, J=8.4, 2.5 Hz, 1H), 8.12 [7.85] (s, 1H), 7.95 (s, 1H), 7.68 (dd, J=8.3, 0.6 Hz, 1H), [7.65] 7.64 (s, 1H), 7.60 [7.56] (dd, J=8.5, 1.3 Hz, 1H), 7.26 (dd, J=8.7, 6.2 Hz, 2H), 6.86 (d, J=8.8 Hz, 2H), 6.04 (s, 2H), 3.69 (s, 3H). ¹⁹F NMR (376 MHz, DMSO) δ −58.96 (s). m/z (ES⁺), [M+H]⁺ 527.9. HPLC (A05) t_{R}=2.69 min.

### Preparation of Compound L-8 (6-chloro-N-[5-(5-methoxy-1H-benzimidazol-2-yl)-1-[(4-methoxyphenyl)-methyl]pyrazol-3-yl]pyridine-3-carboxamide)

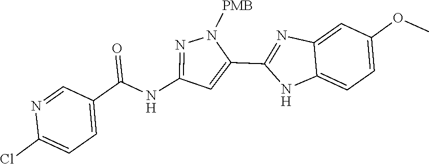

6-Chloropyridine-3-carboxylic acid (236 mg, 1.50 mmol) and HATU (0.570 g, 1.50 mmol) were dissolved in DMF (10 mL) at 0° C. DIEA (0.514 mL, 3.00 mmol) was added, and the mixture was stirred at 0° C. for 5 min. 5-(5-Methoxy-1H-benzimidazol-2-yl)-1-[(4-methoxyphenyl)-methyl]pyrazol-3-amine (J-8) (524 mg, 1.50 mmol) was added. The mixture was stirred at 23° C. for 18 h. EtOAc (100 mL) was added, and the organic phase was washed with water (3×50 mL) and brine (60 mL), dried (Na₂SO₄), filtered, and concentrated. The residue was purified by silica gel chromatography (25 g cartridge) eluting with hexanes and EtOAc (0-100%) to provide the title compound as a solid in a 1:1 mixture of tautomers (545 mg, 74%). ¹H NMR (400 MHz, DMSO) δ 13.00 (s, 1H), 11.42 (s, 1H), 8.99 (dd, J=2.5, 0.6 Hz, 1H), 8.39 (dd, J=8.4, 2.5 Hz, 1H), 7.68 (dd, J=8.4, 0.7 Hz, 1H), 7.60 [7.43] (s, 1H), 7.52 (s, 1H), 7.28-7.16 [6.99] (m, 3H), 6.91-6.82 (m, 3H), 6.02 (s, 2H), 3.82 (s, 3H), 3.69 (s, 3H). m/z (ES⁺), [M+H]⁺ 490.0. HPLC (A05) t_{R}=2.47 min.

### Preparation of Compound L-9 (6-chloro-N-[1-[(4-methoxyphenyl)methyl]-5-[5-(trifluoromethoxy)-1H-benzimidazol-2-yl]pyrazol-3-yl]pyridine-3-carboxamide)

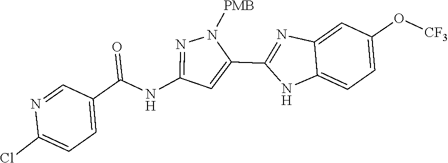

6-Chloropyridine-3-carboxylic acid (236 mg, 1.50 mmol) and HATU (0.570 g, 1.50 mmol) were dissolved in DMF (10 mL) at 0° C. DIEA (0.514 mL, 3.00 mmol) was added, and the mixture was stirred at 0° C. for 5 min. 1-[(4-Methoxyphenyl)methyl]-5-[5-(trifluoromethoxy)-1H-benzimidazol-2-yl]pyrazol-3-amine (J-9) (605 mg, 1.50 mmol) was added. The mixture was stirred at 23° C. for 18 h. EtOAc (100 mL) was added, and the organic phase was washed with water (3×50 mL) and brine (60 mL), dried (Na₂SO₄), filtered, and concentrated. The residue was purified by silica gel chromatography (25 g cartridge) eluting with hexanes and EtOAc (0-100%) to provide the title compound as a solid in a 1:1 mixture of tautomers (653 mg, 80%). ¹H NMR (400 MHz, DMSO) δ 13.47 (s, 1H), 11.47 (s, 1H), 8.99 (dd, J=2.5, 0.6 Hz, 1H), 8.40 (dd, J=8.4, 2.5 Hz, 1H), 7.88-7.71 (m, 1H), 7.70-7.63 [7.58-7.42] (m, 2H), 7.61 (s, 1H), 7.32-7.19 (m, 3H), 6.89-6.82 (m, 2H), 6.03 (s, 2H), 3.69 (s, 3H). m/z (ES⁺), [M+H]⁺ 543.2. HPLC (A05) t_{R}=2.70 min.

### Preparation of Compound L-10 (6-chloro-N-[5-(7-fluoro-5-methoxy-1H-benzimidazol-2-yl)-1-[(4-methoxy-phenyl)methyl]pyrazol-3-yl]pyridine-3-carboxamide)

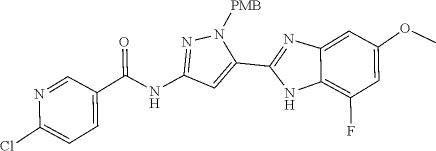

6-Chloropyridine-3-carboxylic acid (343 mg, 2.18 mmol) and HATU (0.828 g, 2.18 mmol) were dissolved in DMF (10 mL) at 0° C. DIEA (0.746 mL, 4.36 mmol) was added, and the mixture was stirred at 0° C. for 5 min. 5-(7-Fluoro-5-methoxy-1H-benzimidazol-2-yl)-1-[(4-methoxyphenyl)methyl]pyrazol-3-amine (J-10) (800 mg, 2.18 mmol) was added. The mixture was warmed to 23° C. and stirred for 18 h. The mixture was heated at 50° C. for 2 h and concentrated. EtOAc (150 mL) was added, and the organic phase was washed with water (3×50 mL) and brine (50 mL), dried (Na₂SO₄), filtered, and concentrated. The residue was purified by silica gel chromatography (40 g cartridge) eluting with DCM and MeOH (0-10%) to provide the title compound as a solid (783 mg, 71%). ¹H NMR (400 MHz, DMSO) δ 13.28 (s, 1H), 11.45 (s, 1H), 8.99 (dd, J=2.5, 0.6 Hz, 1H), 8.40 (dd, J=8.4, 2.5 Hz, 1H), 7.68 (dd, J=8.4, 0.6 Hz, 1H), 7.55 (s, 1H), 7.25 (d, J=8.6 Hz, 2H), 6.91-6.83 (m, 3H), 6.76 (d, J=12.4 Hz, 1H), 5.99 (s, 2H), 3.84 (s, 3H), 3.69 (s, 3H). m/z (ES⁺), [M+H]⁺ 507.2. HPLC (A05) t_{R}=2.56 min.

### Preparation of Compound L-11 (N-[5-(1H-benzimidazol-2-yl)-1-methyl-pyrazol-3-yl]-6-chloro-pyridine-3-carboxamide)

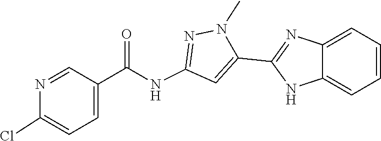 [SMILES: Cn1nc(NC(=O)c2ccc(Cl)nc2)cc1-c1nc2ccccc2[nH]1]

DIEA (1.20 mL, 7.03 mmol) was added to a solution of 6-chloropyridine-3-carboxylic acid (0.443 g, 2.81 mmol) and HATU (1.07 g, 2.81 mmol) in DMF (20 mL) at 0° C. The mixture was stirred at 0° C. for 10 mins. 5-(1H-Benzimidazol-2-yl)-1-methyl-pyrazol-3-amine (J-11) (0.500 g, 2.34 mmol) was added. The mixture was warmed to 23° C. and stirred for 3 days. The mixture was concentrated. Water (100 mL) was added, and the aq. phase was extracted with EtOAc (3×25 mL). The combined organic phases were washed with brine (100 mL), dried (MgSO₄), filtered, and concentrated. The residue was purified by silica gel chromatography (25 g cartridge) eluting with hexanes and EtOAc (0-100%) to provide the title compound as a solid (0.803 g, 97%). ¹H NMR (500 MHz, DMSO) δ 13.10 (s, 1H), 11.41 (s, 1H), 9.01 (dd, J=2.5, 0.6 Hz, 1H), 8.41 (dd, J=8.3, 2.5 Hz, 1H), 7.72 (s, 1H), 7.70 (dd, J=8.3, 0.7 Hz, 1H), 7.54 (d, J=6.1 Hz, 1H), 7.51 (s, 1H), 7.25 (s, 2H), 4.32 (s, 3H). m/z (ES⁺) [M+H]⁺ 535.65. HPLC (A05) t_{R}=2.14 min.

### Preparation of Compound L-12 (6-chloro-N-[5-(5-fluoro-1H-benzimidazol-2-yl)-1-methyl-pyrazol-3-yl]pyridine-3-carboxamide)

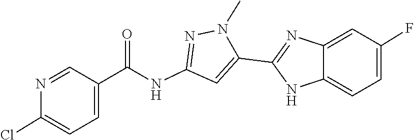 [SMILES: Cn1nc(NC(=O)c2ccc(Cl)nc2)cc1-c1nc2cc(F)ccc2[nH]1]

6-Chloropyridine-3-carboxylic acid (341 mg, 2.16 mmol) and HATU (0.822 g, 2.16 mmol) were dissolved in DMF (10 mL) at 0° C. DIEA (0.740 mL, 4.32 mmol) was added, and the mixture was stirred at 0° C. for 5 min. 5-(5-Fluoro-1H-benzimidazol-2-yl)-1-methyl-pyrazol-3-amine (J-12) (500 mg, 2.16 mmol) was added. The mixture was warmed to 23° C. and stirred for 18 h. The mixture was concentrated. EtOAc (120 mL) was added, and the organic phase was washed with water (2×100 mL) and brine (50 mL), dried (Na₂SO₄), filtered, and concentrated. The residue was purified by silica gel chromatography (25 g cartridge) eluting with hexanes and EtOAc (0-100%) to provide the title compound as a solid in a 1:1 mixture of tautomers (772 mg, 96%). ¹H NMR (400 MHz, DMSO) δ [13.25] 13.24 (s, 1H), 11.42 [11.42] (s, 1H), [9.01] 9.00 (d, J=2.5 Hz, 1H), [8.42] 8.40 (d, J=8.4 Hz, 1H), 7.73 [7.33] (dd, J=8.9, 5.1 Hz, 1H), 7.70 (d, J=8.3 Hz, 1H), 7.57-7.48 (m, 2H), 7.19-7.05 (m, 1H), [4.31] 4.30 (s, 3H). m/z (ES⁺), [M+H]⁺ 371.1. HPLC (A05) t_{R}=2.18 min.

### Preparation of Compound L-13 (6-chloro-N-[5-(7-fluoro-1H-benzimidazol-2-yl)-1-methyl-pyrazol-3-yl]pyridine-3-carboxamide)

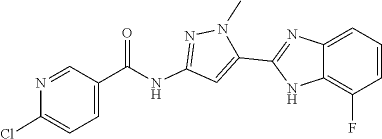 [SMILES: Cn1nc(NC(=O)c2ccc(Cl)nc2)cc1-c1nc2cccc(F)c2[nH]1]

6-Chloropyridine-3-carboxylic acid (341 mg, 2.16 mmol) and HATU (0.822 g, 2.16 mmol) were dissolved in DMF (10 mL) at 0° C. DIEA (0.740 mL, 4.32 mmol) was added, and the mixture was stirred at 0° C. for 5 min. 5-(7-Fluoro-1H-benzimidazol-2-yl)-1-methyl-pyrazol-3-amine (J-13) (500 mg, 2.16 mmol) was added. The mixture was warmed to 23° C. and stirred for 18 h. The mixture was concentrated. EtOAc (120 mL) was added, and the organic phase was washed with water (2×100 mL) and brine (50 mL), dried (Na₂SO₄), filtered, and concentrated. The residue was purified by silica gel chromatography (25 g cartridge) eluting with hexanes and EtOAc (0-100%) to provide the title compound as a solid in a 4:1 mixture of tautomers (643 mg, 80%). 1H NMR (400 MHz, DMSO) δ [13.60] 13.41 (s, 1H), 11.44 [11.41] (s, 1H), 9.01 (dd, J=2.5, 0.6 Hz, 1H), 8.41 (dd, J=8.4, 2.5 Hz, 1H), 7.70 (d, J=8.3 Hz, 1H), [7.66] 7.54 (s, 1H), [7.57] 7.38 (d, J=8.0 Hz, 1H), 7.25 (m, 1H), [7.13] 7.06 (dd, J=11.1, 8.0 Hz, 1H), 4.32 [4.31] (s, 3H). m/z (ES⁺), [M+H]⁺ 371.1. HPLC (A05) t_{R}=2.22 min.

### Preparation of Compound L-14 (6-chloro-N-[5-(7-fluoro-5-methoxy-1H-benzimidazol-2-yl)-1-methyl-pyrazol-3-yl]pyridine-3-carboxamide)

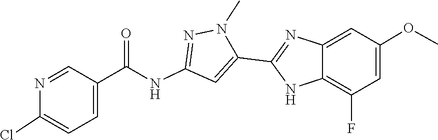 [SMILES: COc1cc(F)c2[nH]c(-c3cc(NC(=O)c4ccc(Cl)nc4)nn3C)nc2c1]

6-Chloropyridine-3-carboxylic acid (343 mg, 2.18 mmol) and HATU (0.828 g, 2.18 mmol) were dissolved in DMF (10 mL) at 0° C. DIEA (0.746 mL, 4.36 mmol) was added, and the mixture was stirred at 0° C. for 5 min. 5-(7-Fluoro-5-methoxy-1H-benzimidazol-2-yl)-1-methyl-pyrazol-3-amine (J-14) (569 mg, 2.18 mmol) was added. The mixture was warmed to 23° C. and stirred for 18 h. The mixture was concentrated. EtOAc (150 mL) was added, and the organic phase was washed with water (3×50 mL) and brine (50 mL), dried (Na₂SO₄), filtered, and concentrated. The residue was purified by silica gel chromatography (40 g cartridge) eluting with DCM and MeOH (0-10%) to provide the title compound as a solid (736 mg, 84%). ¹H NMR (400 MHz, DMSO) δ 13.23 (s, 1H), 11.41 (s, 1H), 9.01 (dd, J=2.5, 0.6 Hz, 1H), 8.41 (dd, J=8.4, 2.5 Hz, 1H), 7.70 (d, J=8.3 Hz, 1H), 7.49 (s, 1H), 6.84 (d, J=2.1 Hz, 1H), 6.75 (dd, J=12.4, 2.1 Hz, 1H), 4.29 (s, 3H), 3.84 (s, 3H). m/z (ES⁺), [M+H]⁺ 401.2. HPLC (A05) t_{R}=2.25 min.

### Preparation of Compound L-15 (N-[5-(1H-benzimidazol-2-yl)-1-[(4-methoxyphenyl)methyl]pyrazol-3-yl]-4-fluoro-benzamide)

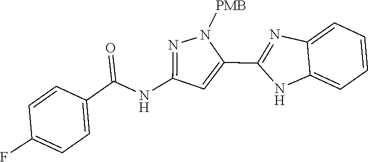

4-Fluorobenzoic acid (351 mg, 2.51 mmol) and HATU (0.952 g, 2.51 mmol) were dissolved in DMF (15 mL) at 0° C. DIEA (0.858 mL, 5.01 mmol) was added, and the mixture was stirred at 0° C. for 5 min. 5-(1H-Benzimidazol-2-yl)-1-[(4-methoxyphenyl)methyl]pyrazol-3-amine (J-1) (800 mg, 2.51 mmol) was added. The mixture was warmed to 23° C. and stirred for 3 days. The mixture was concentrated, and the residue was purified by silica gel chromatography (40 g cartridge) eluting with DCM and MeOH (0-10%) to provide the title compound as a solid (908 mg, 82%). ¹H NMR (500 MHz, DMSO) δ 13.14 (s, 1H), 11.15 (s, 1H), 8.14-8.09 (m, 2H), 7.72 (d, J=7.8 Hz, 1H), 7.57 (s, 1H), 7.54 (d, J=7.8 Hz, 1H), 7.33 (t, J=8.8 Hz, 2H), 7.28 (t, J=7.6 Hz, 1H), 7.23 (t, J=8.0 Hz, 3H), 6.88-6.83 (m, 2H), 6.04 (s, 2H), 3.68 (s, 3H). m/z (ES⁺), [M+H]⁺ 442.4. HPLC (A05) t_{R}=2.48 min.

### Preparation of Compound L-16 (N-[5-(1H-benzimidazol-2-yl)-1-methyl-pyrazol-3-yl]-4-fluoro-benzamide)

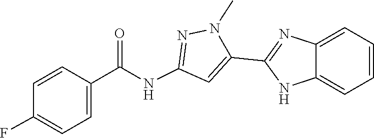 [SMILES: Cn1nc(NC(=O)c2ccc(F)cc2)cc1-c1nc2ccccc2[nH]1]

DIEA (1.20 mL, 7.03 mmol) was added to a solution of 4-fluorobenzoic acid (0.393 g, 2.81 mmol) and HATU (1.07 g, 2.81 mmol) in DMF (20 mL) at 0° C. The mixture was stirred at 0° C. for 10 min. 5-(1H-Benzimidazol-2-yl)-1-methyl-pyrazol-3-amine (I-11) (0.450 g, 2.11 mmol) was added. The mixture was warmed to 23° C. and stirred for 3 days. The mixture was concentrated. Water (100 mL) was added, and the aq. phase was extracted with EtOAc (6×25 mL). The combined organic phases were washed with brine (200 mL), dried (MgSO₄), filtered, and concentrated. The residue was purified by silica gel chromatography (25 g cartridge) eluting with hexanes and EtOAc (0-100%) to provide the title compound as a solid (0.637 g, 90%). ¹H NMR (500 MHz, DMSO) δ 13.08 (s, 1H), 11.11 (s, 1H), 8.15-8.09 (m, 2H), 7.74-7.70 (m, 1H), 7.54 (ddd, J=7.9, 1.2, 0.8 Hz, 1H), 7.50 (s, 1H), 7.35 (ddd, J=9.8, 6.0, 2.6 Hz, 2H), 7.30-7.20 (m, 2H), 4.31 (s, 3H). m/z (ES⁺), [M+H]⁺ 336.1. HPLC (A05) t_{R}=2.21 min.

### Preparation of Compound L-17 (4-fluoro-N-[5-(7-fluoro-5-methoxy-1H-benzimidazol-2-yl)-1-methyl-pyrazol-3-y]benzamide)

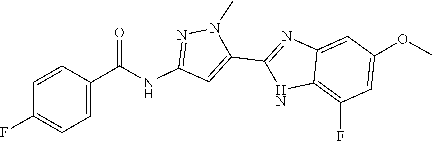 [SMILES: COc1cc(F)c2[nH]c(-c3cc(NC(=O)c4ccc(F)cc4)nn3C)nc2c1]

DIEA (0.655 mL, 3.82 mmol) was added to a solution of 4-fluorobenzoic acid (0.214 g, 1.53 mmol) and HATU (0.581 g, 1.53 mmol) in DMF (20 mL) at 0° C. The mixture was stirred at 0° C. for 10 min. 5-(7-Fluoro-5-methoxy-1H-benzimidazol-2-yl)-1-methyl-pyrazol-3-amine (J-14) (0.300 g, 1.15 mmol) was added. The mixture was warmed to 23° C. and stirred for 3 days. The mixture was concentrated. Water (100 mL) was added, and the aq. phase was extracted with EtOAc (6×25 mL). The combined organic phases were washed with brine (200 mL), dried (MgSO₄), filtered, and concentrated. The residue was purified by silica gel chromatography (25 g cartridge) eluting with hexanes and EtOAc (0-100%) to provide the title compound as a solid (0.410 g, 93%). ¹H NMR (500 MHz, DMSO) δ 13.20 (s, 1H), 11.11 (s, 1H), 8.16-8.09 (m, 2H), 7.48 (s, 1H), 7.38-7.31 (m, 2H), 6.83 (d, J=2.0 Hz, 1H), 6.74 (dd, J=12.4, 2.0 Hz, 1H), 4.28 (s, 3H), 3.83 (s, 3H). m/z (ES⁺) [M+H]⁺ 384.2. HPLC (A05) t_{R}=2.34 min.

### Preparation of Compound L-18 (N-[5-(1H-benzimidazol-2-yl)-1-[(4-methoxyphenyl)methyl]pyrazol-3-yl]-5,6-dichloro-pyridine-3-carboxamide)

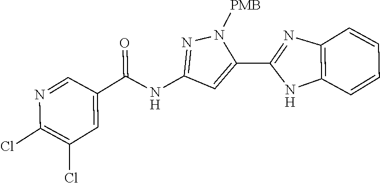

DIEA (0.107 mL, 0.626 mmol) was added to a solution of 5-(1H-benzimidazol-2-yl)-1-[(4-methoxyphenyl) methyl] pyrazol-3-amine (100 mg, 0.313 mmol), and 5,6-dichloropyridine-3-carboxylic acid (60.1 mg, 0.313 mmol) in DMF (3 mL). HATU (119 mg, 0.313 mmol) was added, and the mixture was stirred at 23° C. for 2 h. Water (20 mL) was added. The mixture was filtered, and the solid was dried under high vacuum to provide the title compound (143 mg, 93%) as a solid. ¹H NMR (400 MHz, CDCl₃) δ 9.84 (s, 1H), 8.78 (s, 1H), 8.74 (d, J=2.2 Hz, 1H), 8.26 (d, J=2.2 Hz, 1H), 7.87-7.79 (m, 1H), 7.50-7.43 (m, 1H), 7.34-7.27 (m, 5H), 6.83-6.70 (m, 2H), 5.99 (s, 2H), 3.73 (s, 3H). m/z (ES⁺) [M+H]⁺: 494.9; HPLC (A05) t_{R}=2.64 min.

### Preparation of Compound L-19 ([4-[[5-(1H-benzimidazol-2-yl)-1-[(4-methoxyphenyl)methyl]pyrazol-3-yl]carbamoyl]phenyl]boronic acid)

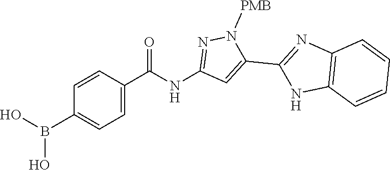

4-Boronobenzoic acid (312 mg, 1.88 mmol) and HATU (0.714 g, 1.88 mmol) were dissolved in DMF (10 mL) at 0° C. DIEA (0.965 mL, 5.64 mmol) was added, and the mixture was stirred at 0° C. for 5 min. 5-(1H-Benzimidazol-2-yl)-1-[(4-methoxyphenyl)methyl]pyrazol-3-amine (J-1) (600 mg, 1.88 mmol) was added. The mixture was warmed to 23° C. and stirred for 18 h. The mixture was concentrated. EtOAc (150 mL) was added to the residue, and the organic phase was washed with water (3×50 mL) and brine (50 mL), dried (Na₂SO₄), filtered, and concentrated. The residue was purified by silica gel chromatography (40 g cartridge) eluting with DCM and MeOH (0-10%) to provide the title compound as a solid (788 mg, 90%). ¹H NMR (400 MHz, DMSO) δ 13.14 (s, 1H), 11.09 (d, J=9.0 Hz, 1H), 8.22 (s, 1H), 8.05 (t, J=10.1 Hz, 1H), 7.98 (d, J=8.4 Hz, 1H), 7.88 (d, J=8.4 Hz, 1H), 7.72 (d, J=7.8 Hz, 1H), 7.59 (d, J=8.0 Hz, 1H), 7.54 (s, 1H), 7.31-7.19 (m, 4H), 6.86 (dd, J=8.9, 2.7 Hz, 2H), 6.04 (s, 2H), 3.69 (d, J=1.4 Hz, 3H), 3.17 (s, 2H). m/z (ES⁺), [M+H]⁺ 467.9. HPLC (A05) t_{R}=2.31 min.

### Preparation of Compound L-20 (N-[5-(1H-benzimidazol-2-yl)-1-(2-methoxyethyl)pyrazol-3-yl]-6-chloro-pyridine-3-carboxamide)

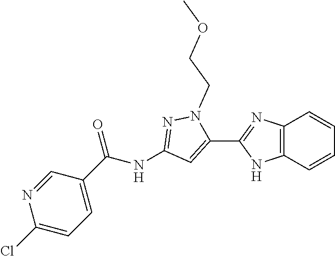 [SMILES: COCCn1nc(NC(=O)c2ccc(Cl)nc2)cc1-c1nc2ccccc2[nH]1]

DIEA (0.542 mL, 3.11 mmol) was added to a solution of HATU (414 mg, 1.09 mmol) and 6-chloropyridine-3-carboxylic acid (465 mg, 2.95 mmol) in DMF (5 mL) at 0° C. The mixture was stirred at 0° C. for 10 min. 5-(1H-Benzimidazol-2-yl)-1-(2-methoxyethyl)pyrazol-3-amine (J-15) (200 mg, 0.777 mmol) was added. The mixture was warmed to 23° C. and stirred for 20 h. Water (50 mL) was added, and the aq. phase was extracted with EtOAc (3×20 mL). The combined organic phases were washed with sat. NaHCO₃ (50 mL) and brine (50 mL), dried (MgSO₄), filtered, and concentrated. The residue was purified by silica gel chromatography (7 g cartridge) eluting with hexanes and EtOAc (0-60%) to provide the title compound as a solid (294 mg, 95%). ¹H NMR (400 MHz, DMSO) δ 13.10 (s, 1H), 11.47 (s, 1H), 9.02 (dd, J=2.5, 0.6 Hz, 1H), 8.42 (dd, J=8.4, 2.5 Hz, 1H), 7.70 (dd, J=11.6, 3.9 Hz, 2H), 7.54 (d, J=6.1 Hz, 2H), 7.25 (dtd, J=14.9, 7.2, 1.3 Hz, 2H), 5.03 (t, J=5.7 Hz, 2H), 3.81 (t, J=5.7 Hz, 2H), 3.20 (s, 3H). m/z (ES⁺), [M−H]⁻ 395.6. HPLC (A05) t_{R}=2.23 min.

### Preparation of Compound L-21 (N-[1-allyl-5-(1H-benzimidazol-2-yl)pyrazol-3-yl]-6-chloro-pyridine-3-carboxamide)

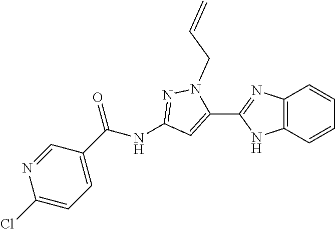 [SMILES: C=CCn1nc(NC(=O)c2ccc(Cl)nc2)cc1-c1nc2ccccc2[nH]1]

DIEA (0.110 mL, 0.634 mmol) was added to a solution of 6-chloropyridine-3-carboxylic acid (103 mg, 0.652 mmol) and HATU (248 mg, 0.652 mmol) in DMF (6 mL) at 0° C. The mixture was stirred at 0° C. for 10 min. 1-Allyl-5-(1H-benzimidazol-2-yl)pyrazol-3-amine (J-16) (130 mg, 0.502 mmol) was added. The mixture was stirred at 23° C. for 90 h. Water (60 mL) was added, and the aq. phase was extracted with EtOAc (3×15 mL). The combined organic phases were washed with aq. NaHCO₃ (50 mL) and brine (50 mL), dried (MgSO₄), filtered, and concentrated. The residue was purified by silica gel chromatography (12 g cartridge) eluting with hexanes and EtOAc (20-60%) to provide the title compound as a solid (142 mg, 69%). m/z (ES⁺), [M−H]⁻ 377.6. HPLC (A05) t_{R}=2.31 min.

### Preparation of Compound L-22 (N-[5-(1H-benzimidazol-2-yl)-1-[(4-methoxyphenyl)methyl]pyrazol-3-yl]-6-chloro-pyridazine-3-carboxamide)

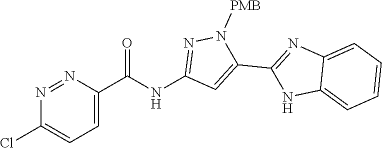

6-Chloropyridazine-3-carboxylic acid (248 mg, 1.57 mmol) and HATU (0.595 g, 1.57 mmol) were dissolved in DMF (10 mL) at 0° C. DIEA (0.536 mL, 3.13 mmol) was added, and the mixture was stirred at 0° C. for 5 min. 5-(1H-Benzimidazol-2-yl)-1-[(4-methoxyphenyl)methyl]pyrazol-3-amine (J-1) (500 mg, 1.57 mmol) was added. The mixture was warmed to 23° C. and stirred for 18 h. The mixture was concentrated. EtOAc (150 mL) was added, and the organic phase was washed with water (3×50 mL) and brine (50 mL), dried (Na₂SO₄), filtered, and concentrated. The residue was purified by silica gel chromatography (40 g cartridge) eluting with DCM and MeOH (0-5%) to provide the title compound as a solid (426 mg, 59%). ¹H NMR (400 MHz, DMSO) δ 13.17 (s, 1H), 11.29 (s, 1H), 8.33 (d, J=8.9 Hz, 1H), 8.17 (d, J=8.9 Hz, 1H), 7.72 (s, 1H), 7.60-7.51 (m, 2H), 7.26 (d, J=8.8 Hz, 4H), 6.88-6.82 (m, 2H), 6.05 (s, 2H), 3.68 (s, 3H). m/z (ES⁺), [M+H]⁺ 461.0. HPLC (A05) t_{R}=2.51 min.

### Preparation of Compound L-23 (N-[5-(1H-benzimidazol-2-yl)-1-[(4-methoxyphenyl)methyl]pyrazol-3-yl]-5-chloro-pyrazine-2-carboxamide)

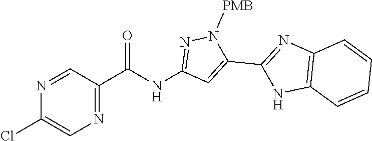

5-Chloropyrazine-2-carboxylic acid (248 mg, 1.57 mmol) and HATU (0.595 g, 1.57 mmol) were dissolved in DMF (10 mL) at 0° C. DIEA (0.536 mL, 3.13 mmol) was added, and the mixture was stirred at 0° C. for 5 min. 5-(1H-Benzimidazol-2-yl)-1-[(4-methoxyphenyl)-methyl]pyrazol-3-amine (I-1) (500 mg, 1.57 mmol) was added. The mixture was warmed to 23° C. and stirred for 18 h. The mixture was concentrated. EtOAc (150 mL) was added, and the organic phase was washed with water (3×50 mL) and brine (50 mL), dried (Na₂SO₄), filtered, and concentrated. The residue was purified by silica gel chromatography (40 g cartridge) eluting with DCM and MeOH (0-5%) to provide the title compound as a solid (400 mg, 56%). ¹H NMR (400 MHz, DMSO) δ 13.17 (s, 1H), 11.29 (s, 1H), 8.33 (d, J=8.9 Hz, 1H), 8.17 (d, J=8.9 Hz, 1H), 7.72 (s, 1H), 7.60-7.51 (m, 2H), 7.26 (d, J=8.8 Hz, 4H), 6.88-6.82 (m, 2H), 6.05 (s, 2H), 3.68 (s, 3H). m/z (ES⁺), [M+H]⁺ 461.1. HPLC (A05) t_{R}=2.59 min.

### Preparation of Compound L-24 (N-[5-(1H-benzimidazol-2-yl)-1-[(4-methoxyphenyl)methyl]pyrazol-3-yl]-2-chloro-pyrimidine-5-carboxamide)

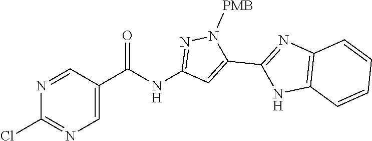

2-Chloropyrimidine-5-carboxylic acid (248 mg, 1.57 mmol) and HATU (0.595 g, 1.57 mmol) were dissolved in DMF (10 mL) at 0° C. DIEA (0.536 mL, 3.13 mmol) was added, and the mixture was stirred at 0° C. for 5 min. 5-(1H-Benzimidazol-2-yl)-1-[(4-methoxyphenyl)-methyl]pyrazol-3-amine (J-1) (500 mg, 1.57 mmol) was added. The mixture was warmed to 23° C. and stirred for 18 h. The mixture was concentrated. EtOAc (150 mL) was added, and the organic phase was washed with water (3×50 mL) and brine (50 mL), dried (Na₂SO₄), filtered, and concentrated. The residue was purified by silica gel chromatography (40 g cartridge) eluting with DCM and MeOH (0-5%) to provide the title compound as a solid (300 mg, 42%). ¹H NMR (400 MHz, DMSO) δ 13.18 (s, 1H), 11.61 (s, 1H), 9.26 (s, 2H), 7.73 (d, J=7.5 Hz, 1H), 7.58 (s, 1H), 7.55 (d, J=7.4 Hz, 1H), 7.32-7.20 (m, 4H), 6.89-6.83 (m, 2H), 6.05 (s, 2H), 3.69 (s, 3H). m/z (ES⁺), [M+H]⁺ 460.1. HPLC (A05) t_{R}=2.45 min.

### Preparation of Compound L-25 (N-[5-(1H-benzimidazol-2-yl)-1-[(4-methoxyphenyl)methyl]pyrazol-3-yl]-5-bromo-pyridine-2-carboxamide)

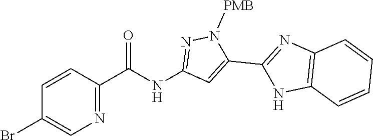

5-Bromopyridine-2-carboxylic acid (443 mg, 2.19 mmol) and HATU (0.833 g, 2.19 mmol) were dissolved in DMF (10 mL) at 0° C. DIEA (0.750 mL, 4.38 mmol) was added, and the mixture was stirred at 0° C. for 5 min. 5-(1H-Benzimidazol-2-yl)-1-[(4-methoxyphenyl)-methyl]pyrazol-3-amine (J-1) (700 mg, 2.19 mmol) was added. The mixture was warmed to 23° C. and stirred for 18 h. The mixture was concentrated. EtOAc (150 mL) was added, and the organic phase was washed with water (3×50 mL) and brine (50 mL), dried (Na₂SO₄), filtered, and concentrated. The residue was purified by silica gel chromatography (40 g cartridge) eluting with DCM and MeOH (0-5%) to provide the title compound as a solid (974 mg, 88%). ¹H NMR (500 MHz, DMSO) δ 13.15 (s, 1H), 10.66 (s, 1H), 8.85 (dd, J=2.3, 0.7 Hz, 1H), 8.33 (dd, J=8.4, 2.3 Hz, 1H), 8.09 (dd, J=8.3, 0.7 Hz, 1H), 7.73 (s, 1H), 7.59-7.51 (m, 2H), 7.26 (d, J=8.8 Hz, 4H), 6.87-6.82 (m, 2H), 6.03 (s, 2H), 3.68 (s, 3H). m/z (ES⁺), [M+H]⁺ 503.9. HPLC (A05) t_{R}=2.74 min.

### Preparation of Compound L-26 (N-[1-allyl-5-(1H-benzimidazol-2-yl)pyrazol-3-yl]-4-fluoro-benzamide)

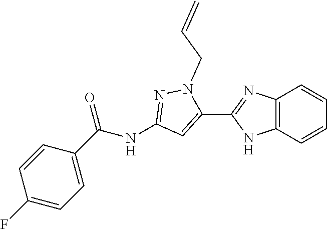 [SMILES: C=CCn1nc(NC(=O)c2ccc(F)cc2)cc1-c1nc2ccccc2[nH]1]

DIEA (0.102 mL, 0.586 mmol) was added to a solution of 4-fluorobenzoic acid (70.3 mg, 0.502 mmol) and HATU (211 mg, 0.555 mmol) in DMF (4 mL) at 0° C. The mixture was stirred at 0° C. for 10 min. 1-Allyl-5-(1H-benzimidazol-2-yl)pyrazol-3-amine (70.3 mg, 0.283 mmol) (J-16) was added. The mixture was stirred at 23° C. for 18 h. Water (40 mL) was added, and the aq. phase was extracted with EtOAc (3×10 mL). The combined organic phases were washed with sat. NaHCO₃ (50 mL) and brine (50 mL), dried (MgSO₄), filtered, and concentrated. The residue was purified by silica gel chromatography (12 g cartridge) eluting with hexanes and EtOAc (20-60%) to provide the title compound as an oil (109 mg, 60%). ¹H NMR (400 MHz, DMSO) δ 13.08 (s, 1H), 11.16 (s, 1H), 8.14 (dd, J=8.9, 5.5 Hz, 2H), 7.71 (d, J=7.7 Hz, 1H), 7.56 (s, 1H), 7.53 (d, J=8.1 Hz, 1H), 7.37-7.20 (m, 4H), 6.09 (ddd, J=15.7, 10.7, 5.5 Hz, 1H), 5.49 (d, J=5.5 Hz, 2H), 5.15 (dd, J=10.3, 1.5 Hz, 1H), 5.08 (d, J=17.1 Hz, 1H). m/z (ES⁻), [M−H]⁻ 360.6. HPLC (A05) t_{R}=2.39 min.

### Preparation of Compound L-27 (N-[5-(1H-benzimidazol-2-yl)-1-methyl-pyrazol-3-yl]-5-(triazolo[4,5-b]pyridin-3-yloxy)pyrazine-2-carboxamide)

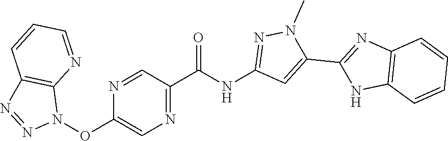 [SMILES: Cn1nc(NC(=O)c2cnc(On3nnc4cccnc43)cn2)cc1-c1nc2ccccc2[nH]1]

5-Chloropyrazine-2-carboxylic acid (37.2 mg, 0.234 mmol) and HATU (0.0892 g, 0.234 mmol) were dissolved in DMF (1 mL) at 0° C. DIEA (0.0803 mL, 0.469 mmol) was added, and the mixture was stirred at 0° C. for 5 min. 5-(1H-Benzimidazol-2-yl)-1-methyl-pyrazol-3-amine (J-11) (50 mg, 0.234 mmol) was added. The mixture was warmed to 23° C. and stirred for 18 h. The mixture was concentrated, and the residue was purified by silica gel chromatography (12 g cartridge) eluting with DCM and MeOH (0-10%). The purified residue was dissolved in EtOAc (150 mL). The organic phase was washed with water (50 mL), sat. NaHCO₃ (2×50 mL), water (50 mL), and brine (50 mL). the organic phase was dried (Na₂SO₄), filtered, and concentrated to provide the title compound as a solid (77 mg, 72%). ¹H NMR (400 MHz, DMSO) δ 13.11 (s, 1H), 10.92 (s, 1H), 9.23 (d, J=1.3 Hz, 1H), 8.84 (dd, J=4.5, 1.3 Hz, 1H), 8.81-8.77 (m, 2H), 7.76-7.65 (m, 2H), 7.59-7.51 (m, 1H), 7.48 (s, 1H), 7.28-7.22 (m, 2H), 4.31 (s, 3H). m/z (ES⁺), [M+H]⁺ 454.7. HPLC (A05) t_{R}=2.25 min.

### Preparation of Compound L-28 (N-[5-(1H-benzimidazol-2-yl)-1-methyl-pyrazol-3-yl]-2-(triazolo[4,5-b]pyridin-3-yloxy)pyrimidine-5-carboxamide)

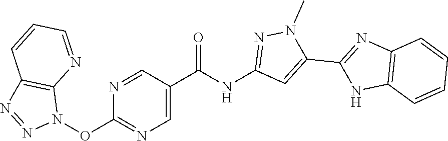 [SMILES: Cn1nc(NC(=O)c2cnc(On3nnc4cccnc43)nc2)cc1-c1nc2ccccc2[nH]1]

2-Chloropyrimidine-5-carboxylic acid (37.2 mg, 0.234 mmol) and HATU (0.0892 g, 0.234 mmol) were dissolved in DMF (1 mL) at 0° C. DIEA (0.0803 mL, 0.469 mmol) was added, and the mixture was stirred at 0° C. for 5 min. 5-(1H-Benzimidazol-2-yl)-1-methyl-pyrazol-3-amine (J-11) (50 mg, 0.234 mmol) was added. The mixture was warmed to 23° C. and stirred for 18 h. The mixture was concentrated, and the residue was purified by silica gel chromatography (12 g cartridge) eluting with DCM and MeOH (0-10%). The purified residue was dissolved in EtOAc (150 mL). The organic phase was washed with water (50 mL), sat. NaHCO₃ (2×50 mL), water (50 mL), and brine (50 mL). The organic phase was dried (Na₂SO₄), filtered, and concentrated to provide the title compound as a solid (70.7 mg, 66%). ¹H NMR (400 MHz, DMSO) δ 13.09 (s, 1H), 11.49 (s, 1H), 9.25 (s, 2H), 8.84 (dd, J=4.5, 1.4 Hz, 1H), 8.79 (dd, J=8.5, 1.4 Hz, 1H), 7.74-7.67 (m, 2H), 7.53 (d, J=7.5 Hz, 1H), 7.50 (s, 1H), 7.30-7.21 (m, 2H), 4.31 (s, 3H). m/z (ES⁺), [M+H]⁺ 454.7. HPLC (A05) t_{R}=2.19 min.

### Preparation of Compound L-29 (N-[5-(1H-benzimidazol-2-yl)-1-methyl-pyrazol-3-yl]-2-(triazolo[4,5-b]pyridin-3-yloxy)pyrimidine-5-carboxamide)

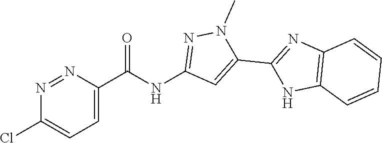 [SMILES: Cn1nc(NC(=O)c2ccc(Cl)nn2)cc1-c1nc2ccccc2[nH]1]

6-Chloropyridazine-3-carboxylic acid (37.2 mg, 0.234 mmol) and HATU (0.0892 g, 0.234 mmol) were dissolved in DMF (1 mL) at 0° C. DIEA (0.0803 mL, 0.469 mmol) was added, and the mixture was stirred at 0° C. for 5 min. 5-(1H-Benzimidazol-2-yl)-1-methyl-pyrazol-3-amine (J-11) (50 mg, 0.234 mmol) was added. The mixture was warmed to 23° C. and stirred for 18 h. The mixture was concentrated, and the residue was purified by silica gel chromatography (12 g cartridge) eluting with DCM and MeOH (0-5%) to provide the title compound as a solid (60.2 mg, 73%). ¹H NMR (400 MHz, DMSO) δ 13.10 (s, 1H), 11.26 (s, 1H), 8.35 (d, J=8.9 Hz, 1H), 8.18 (d, J=8.9 Hz, 1H), 7.78-7.63 (m, 1H), 7.58 (s, 1H), 7.50 (s, 1H), 7.26 (dt, J=7.8, 5.0 Hz, 2H), 4.32 (s, 3H). m/z (ES⁺), [M+H]⁺ 354.6. HPLC (A05) t_{R}=2.17 min.

### EXAMPLE B8

### Preparation of Compounds M-1 to M-5

### Preparation of Compound M-1 (methyl 5-amino-2-[(4-methoxyphenyl)methyl]pyrazole-3-carboxylate)

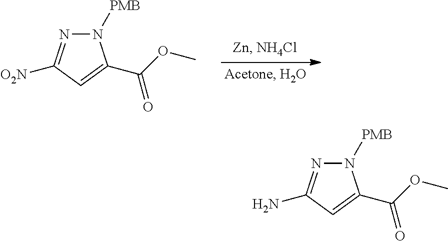

Zinc dust (5.61 g, 85.8 mmol) was added in portions to a mixture of methyl 2-[(4-methoxyphenyl)methyl]-5-nitro-pyrazole-3-carboxylate (5.00 g, 17.2 mmol) and NH₄Cl (9.18 g, 172 mmol) in acetone (320 mL) and water (65 mL). The internal temperature was kept between 20-23° C. during the addition by cooling with an ice bath when required. The suspension was stirred at 23° C. for 4 h. The mixture was filtered through Celite, and the cake was washed with acetone. The filtrate was concentrated. The residue was diluted with water (250 mL), and the aq. phase was extracted with EtOAc (3×250 mL). The combined organic phases were washed with brine (250 mL), dried (MgSO₄), filtered, and concentrated. The residue was diluted with hexanes. The mixture was filtered, and the solid was dried under high vacuum to provide the title compound as a solid (3.87 g, 86%). ¹H NMR (500 MHz, DMSO) δ 7.18-7.03 (m, 2H), 6.97-6.76 (m, 2H), 6.01 (s, 1H), 5.37 (s, 2H), 4.91 (s, 2H), 3.78 (s, 3H), 3.72 (s, 3H). m/z (ES⁺), [M+H]⁺ 262.2. HPLC (A05) t_{R}=2.08 min.

### Representative Synthetic Scheme for the Syntheses of Compounds M-2 to M-4.

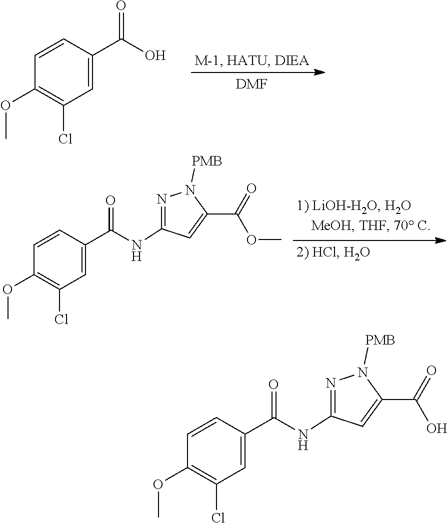

### Preparation of Compound M-2.

### Step 1: methyl 5-[(3-chloro-4-methoxy-benzoyl)amino]-2-[(4-methoxy-phenyl)-methyl]pyrazole-3-carboxylate

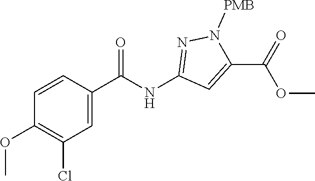

DIEA (2.33 mL, 13.4 mmol) was added to a solution of HATU (1.60 g, 4.21 mmol) and 3-chloro-4-methoxy-benzoic acid (0.750 g, 4.02 mmol) in DMF (15 mL) at 0° C. The mixture was stirred at 0° C. for 10 min. Methyl 5-amino-2-[(4-methoxyphenyl)methyl]pyrazole-3-carboxylate (M-1) (1.00 g, 3.83 mmol) was added. The mixture was warmed to 23° C. and stirred for 3 days. Water (150 mL) was added, and the mixture was stirred at 23° C. for 10 min. The mixture was filtered, and the solid was washed with sat. NaHCO₃ (50 mL) and water (50 mL). The solid was purified by silica gel chromatography (40 g cartridge) eluting with hexanes and EtOAc (0-100%) to provide the title compound as a solid (1.20 g, 73%). ¹H NMR (500 MHz, DMSO) δ 11.12 (s, 1H), 8.14 (d, J=2.2 Hz, 1H), 8.04 (dd, J=8.7, 2.2 Hz, 1H), 7.26 (d, J=8.8 Hz, 1H), 7.24 (s, 1H), 7.20-7.16 (m, 2H), 6.92-6.88 (m, 2H), 5.60 (s, 2H), 3.94 (s, 3H), 3.86 (s, 3H), 3.73 (s, 3H). m/z (ES⁺), [M+H]⁺ 430.6. HPLC (A05) t_{R}=2.58 min.

### Step 2: 5-[(3-chloro-4-methoxy-benzoyl)amino]-2-[(4-methoxyphenyl)-methyl]-pyrazole-3-carboxylic acid

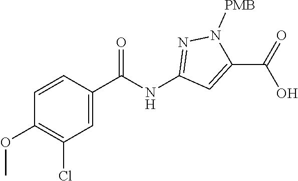

LiOH—H₂O (0.351 g, 8.37 mmol) was added to a mixture of methyl 5-[(3-chloro-4-methoxy-benzoyl)amino]-2-[(4-methoxyphenyl)methyl]pyrazole-3-carboxylate (1.20 g, 2.79 mmol) in MeOH (7.50 mL), THF (7.50 mL), and water (2.50 mL). The mixture was stirred at 70° C. for 1 h and concentrated. Water (50 mL) was added, and the aq. phase was acidified to pH 2 with conc. HCl. The mixture was filtered, and the solid was dried in a vacuum oven at 45° C. for 4 h to provide the title compound as a solid (1.14 g, 98%). ¹H NMR (500 MHz, DMSO) δ 13.50 (s, 1H), 11.06 (s, 1H), 8.14 (s, 1H), 8.04 (d, J=8.1 Hz, 1H), 7.26 (d, J=8.5 Hz, 1H), 7.21-7.06 (m, 3H), 6.90 (d, J=8.2 Hz, 2H), 5.61 (s, 2H), 3.94 (s, 3H), 3.73 (s, 3H). m/z (ES), [M−H]⁻ 414.5. HPLC (A05) t_{R}=2.10 min.

### Preparation of Compound M-3.

### Step 1: N-[5-(1H-benzimidazol-2-yl)-1-[(4-methoxyphenyl)methyl]pyrazol-3-yl]-3-chloro-4-(3-methoxypropoxy)benzamide

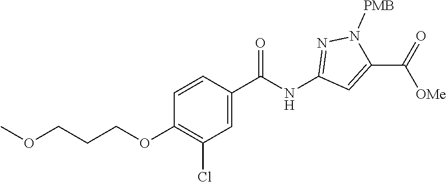

3-Chloro-4-(3-methoxypropoxy)benzoic acid (K-7) (80 mg, 0.327 mmol) and HATU (0.124 g, 0.327 mmol) were dissolved in DMF (3 mL) at 0° C. DIEA (0.112 mL, 0.654 mmol) was added, and the mixture was stirred at 0° C. for 5 min. 5-(1H-Benzimidazol-2-yl)-1-[(4-methoxyphenyl)methyl]pyrazol-3-amine (M-1) (104 mg, 0.327 mmol) was added. The mixture was warmed to 23° C. and stirred for 18 h. EtOAc (200 mL) was added, and the organic phase was washed with water (4×50 mL) and brine (50 mL), dried (MgSO₄), filtered and concentrated to provide the title compound as a solid (176 mg, 99%). ¹H NMR (400 MHz, DMSO) δ 13.13 (s, 1H), 11.07 (s, 1H), 8.15 (d, J=2.2 Hz, 1H), 8.04 (dd, J=8.7, 2.2 Hz, 1H), 7.72 (d, J=7.4 Hz, 1H), 7.57-7.52 (m, 2H), 7.32-7.20 (m, 5H), 6.88-6.83 (m, 2H), 6.03 (s, 2H), 4.20 (t, J=6.3 Hz, 2H), 3.68 (s, 3H), 3.51 (t, J=6.3 Hz, 2H), 3.26 (s, 3H), 2.05-1.97 (m, 2H). m/z (ES⁺), [M+H]⁺ 547.6. HPLC (A05) t_{R}=2.69 min.

### Step 2: 5-[[3-chloro-4-(3-methoxypropoxy)benzoyl]amino]-2-[(4-methoxyphenyl)-methyl]pyrazole-3-carboxylic acid

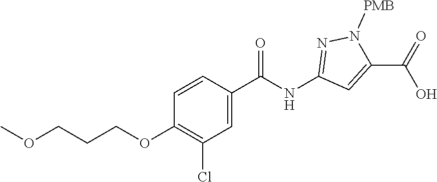

Methyl 5-[[3-chloro-4-(3-methoxypropoxy)benzoyl]amino]-2-[(4-methoxyphenyl)methyl]-pyrazole-3-carboxylate (353 mg, 0.723 mmol) was dissolved in THF (2.50 mL) and MeOH (2.50 mL). 2.50 M aq. NaOH (0.868 mL, 2.17 mmol) was added. The mixture was heated at 60° C. for 1 h. The mixture was concentrated. Water (10 mL) was added. The mixture was acidified to pH 2-3 with aq. HCl (1 M), at which point a precipitate formed. The mixture was filtered, and the solid was dried under high vacuum to provide the title compound as a solid (305 mg, 89%). ¹H NMR (400 MHz, DMSO) δ 13.50 (s, 1H), 11.05 (s, 1H), 8.12 (d, J=2.3 Hz, 1H), 8.00 (dd, J=8.7, 2.3 Hz, 1H), 7.24 (d, J=8.8 Hz, 1H), 7.19-7.13 (m, 3H), 6.91-6.86 (m, 2H), 5.60 (s, 2H), 4.19 (t, J=6.3 Hz, 2H), 3.72 (s, 3H), 3.50 (t, J=6.2 Hz, 2H), 3.25 (s, 3H), 1.99 (p, J=6.3 Hz, 2H). m/z (ES), [M−H]⁻ 472.4. HPLC (A05) t_{R}=2.19 min.

### Preparation of Compound M-4.

### Step 1: methyl 5-[[3-chloro-4-(2-methoxyethoxy)benzoyl]amino]-2-[(4-methoxyphenyl)methyl]pyrazole-3-carboxylate

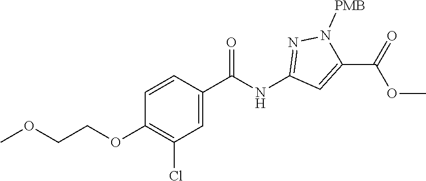

3-Chloro-4-(2-methoxyethoxy)benzoic acid (K-1) (170 mg, 0.737 mmol) and HATU (0.280 g, 0.737 mmol) were dissolved in DMF (6 mL) at 0° C. DIEA (0.252 mL, 1.47 mmol) was added, and the mixture was stirred at 0° C. for 5 min. Methyl 5-amino-2-[(4-methoxyphenyl)-methyl]pyrazole-3-carboxylate (M-1) (193 mg, 0.737 mmol) was added, and the mixture was stirred at 23° C. for 18 h. EtOAc (100 mL) was added, and the organic phase was washed with water (3×50 mL) and brine (60 mL), dried (MgSO₄), filtered and concentrated. The residue was purified by silica gel chromatography (25 g cartridge) eluting with hexanes and EtOAc (0-60%) to provide the title compound as a solid (266 mg, 76%). ¹H NMR (500 MHz, DMSO) δ 11.11 (s, 1H), 8.13 (d, J=2.2 Hz, 1H), 8.00 (dd, J=8.7, 2.2 Hz, 1H), 7.26 (d, J=8.8 Hz, 1H), 7.23 (s, 1H), 7.17 (d, J=8.8 Hz, 2H), 6.91-6.87 (m, 2H), 5.59 (s, 2H), 4.30-4.26 (m, 2H), 3.85 (s, 3H), 3.73-3.70 (m, 5H), 3.34 (s, 3H). m/z (ES⁺), [M+H]⁺ 574.4. HPLC (A05) t_{R}=2.57 min.

### Step 2: 5-[[3-chloro-4-(2-methoxyethoxy)benzoyl]amino]-2-[(4-methoxyphenyl)-methyl]pyrazole-3-carboxylic acid

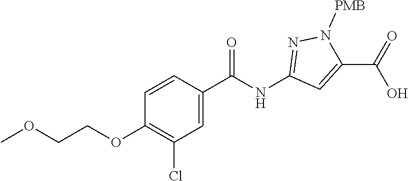

Methyl 5-[[3-chloro-4-(2-methoxyethoxy)benzoyl]amino]-2-[(4-methoxyphenyl)methyl]-pyrazole-3-carboxylate (260 mg, 0.549 mmol) was dissolved in THF (1.50 mL) and MeOH (1.50 mL). 2.50 M aq. LiOH—H₂O (0.658 mL, 1.65 mmol) was added. The mixture was heated at 60° C. and stirred for 20 min. The mixture was partially concentrated and water (10 mL) was added. The mixture was acidified to pH 1-2 with aq. HCl (1 M), at which point a precipitate formed. The mixture was filtered, and the solid was lyophilized to provide the title compound as a solid (246 mg, 98%). ¹H NMR (400 MHz, DMSO) δ 13.50 (s, 1H), 11.05 (s, 1H), 8.12 (d, J=2.2 Hz, 1H), 8.00 (dd, J=8.7, 2.3 Hz, 1H), 7.26 (d, J=8.8 Hz, 1H), 7.18-7.12 (m, 3H), 6.92-6.86 (m, 2H), 5.60 (s, 2H), 4.30-4.24 (m, 2H), 3.74-3.69 (m, 5H), 3.34 (s, 3H). m/z (ES⁺), [M+H]⁺ 460.5. HPLC (A05) t_{R}=2.11 min.

### Preparation of Compound M-5.

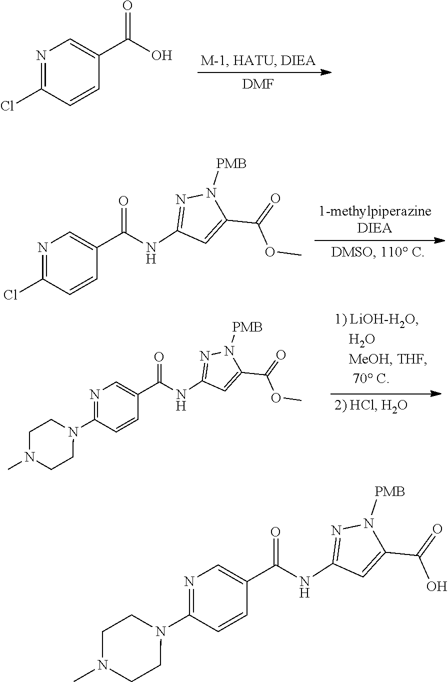

### Step 1: methyl 5-[(6-chloropyridine-3-carbonyl)amino]-2-[(4-methoxyphenyl)-methyl]pyrazole-3-carboxylate

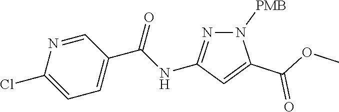

DIEA (2.33 mL, 13.4 mmol) was added to a solution of HATU (1.60 g, 4.21 mmol) and 6-chloropyridine-3-carboxylic acid (0.560 mL, 4.02 mmol) in DMF (15 mL) at 0° C. The mixture was stirred at 0° C. for 30 min. Methyl 5-amino-2-[(4-methoxyphenyl)methyl]pyrazole-3-carboxylate (M-1) (1.00 g, 3.83 mmol) was added. The mixture was warmed to 23° C., stirred for 18 h, and poured into water (100 mL). The aq. phase was extracted with EtOAc (3×100 mL). The combined organic phases were washed with sat. NaHCO₃ (2×100 mL) and brine (100 mL), dried (MgSO₄), filtered, and concentrated. The residue was purified by silica gel chromatography (40 g cartridge) eluting with hexanes and EtOAc (0-100%). The purified residue was diluted in hexanes (50 mL) and stirred at 23° C. for 5 min. The mixture was filtered, and the solid was dried under high vacuum to provide the title compound as a solid (0.684 g, 45%). ¹H NMR (500 MHz, DMSO) δ 11.49 (s, 1H), 8.97 (d, J=2.3 Hz, 1H), 8.38 (dd, J=8.4, 2.5 Hz, 1H), 7.68 (d, J=8.4 Hz, 1H), 7.26 (s, 1H), 7.22-7.13 (m, 2H), 6.97-6.86 (m, 2H), 5.61 (s, 2H), 3.87 (s, 3H), 3.73 (s, 3H). m/z (ES⁺), [M+H]⁺ 401.3. HPLC (A05) t_{R}=2.44 min.

### Step 2: methyl 2-[(4-methoxyphenyl)methyl]-5-[[6-(4-methylpiperazin-1-yl)pyridine-3-carbonyl]amino]pyrazole-3-carboxylate

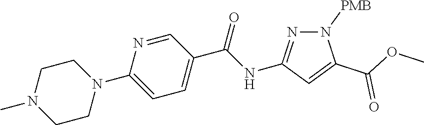

Methyl 5-[(6-chloropyridine-3-carbonyl)amino]-2-[(4-methoxyphenyl)methyl]pyrazole-3-carbo-xylate (480 mg, 1.20 mmol), DIEA (0.615 mL, 3.59 mmol), and 1-methylpiperazine (0.266 mL, 2.40 mmol) were dissolved in DMSO (6 mL). The mixture was heated at 110° C. for 5 h. After cooling to 23° C., water (50 mL) was added, and the aq. phase was extracted with EtOAc (3×50 mL). The combined organic phases were washed with brine (50 mL), dried (MgSO₄), filtered, and concentrated. The residue was purified by silica gel chromatography (40 g cartridge) with DCM and MeOH (0-10%) eluent to provide the title compound as a solid (258 mg, 46%). ¹H NMR (500 MHz, CDCl₃) δ 8.67 (d, J=2.1 Hz, 1H), 8.27 (s, 1H), 7.95 (dd, J=9.0, 2.6 Hz, 1H), 7.39 (s, 1H), 7.25-7.17 (m, 2H), 6.90-6.76 (m, 2H), 6.64 (d, J=8.8 Hz, 1H), 5.61 (s, 2H), 3.87 (s, 3H), 3.77 (s, 3H), 3.74-3.60 (m, 4H), 2.59-2.42 (m, 4H), 2.35 (s, 3H). m/z (ES⁺), [M+H]⁺ 465.5. HPLC (A05) t_{R}=1.97 min.

### Step 3: 2-[(4-methoxyphenyl)methyl]-5-[[6-(4-methylpiperazin-1-yl)pyridine-3-carbonyl]amino]pyrazole-3-carboxylic acid

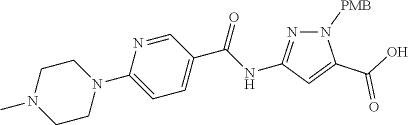

LiOH—H₂O (37 mg, 0.881 mmol) was added to a mixture of methyl 2-[(4-methoxyphenyl)methyl]-5-[[6-(4-methylpiperazin-1-yl)pyridine-3-carbonyl]amino]pyrazole-3-carboxylate (341 mg, 0.734 mmol) in MeOH (0.750 mL), THF (0.750 mL), and water (0.125 mL). The mixture was heated at 70° C. for 1.5 h. After cooling to 23° C., the mixture was partially concentrated. Water (10 mL) was added, and the aq. phase was acidified to pH 6 with 1M aq. HCl, at which point a precipitate formed. The mixture was filtered, and the solid was dried under high vacuum to provide the title compound as a solid (290 mg, 88%). ¹H NMR (500 MHz, DMSO) δ 10.80 (s, 1H), 8.76 (d, J=2.1 Hz, 1H), 8.12 (dd, J=9.1, 2.5 Hz, 1H), 7.18-7.14 (m, 2H), 7.12 (s, 1H), 6.91-6.86 (m, 3H), 5.62 (s, 2H), 3.72 (s, 3H), 3.68-3.61 (m, 4H), 2.50-2.48 (m, 4H), 2.29 (s, 3H). m/z (ES⁺), [M+H]⁺ 451.0. HPLC (A05) t_{R}=1.67 min.

### EXAMPLE B9

### Preparation of Compound N-1

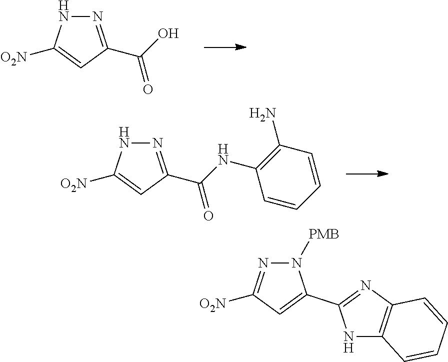

### Step 1: N-(2-aminophenyl)-5-nitro-1H-pyrazole-3-carboxamide

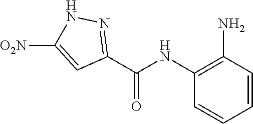 [SMILES: Nc1ccccc1NC(=O)c1cc([N+](=O)[O-])[nH]n1]

5-Nitro-1H-pyrazole-3-carboxylic acid (3.14 g, 20.0 mmol) and HATU (7.60 g, 20.0 mmol) were dissolved in DMF (100 mL) at 0° C. DIEA (6.85 mL, 40.0 mmol) was added, and the mixture was stirred at 0° C. for 5 min. o-Phenylenediamine (2.16 g, 20.0 mmol) was added. The mixture was stirred at 0° C. for 5 min, warmed to 23° C., and stirred for 18 h. The mixture was concentrated. Water (100 mL) was added, and the aq. phase was extracted with EtOAc (3×100 mL). The combined organic phases were washed with water (100 mL) and brine (100 mL), dried (MgSO₄), filtered, and concentrated. The residue was diluted in MeCN (100 mL) and stirred for 5 min. The mixture was filtered, and the solid was washed with hexanes and dried under high vacuum to provide the title compound as a solid (3.32 g, 67%). ¹H NMR (500 MHz, DMSO) δ 14.88 (br, 1H), 9.89 (s, 1H), 7.83 (s, 1H), 7.15 (dd, J=7.8, 1.4 Hz, 1H), 7.04-6.99 (m, 1H), 6.79 (dd, J=8.1, 1.3 Hz, 1H), 6.60 (td, J=7.7, 1.4 Hz, 1H), 5.07 (br, 2H). m/z (ES⁺), [M−H]⁻ 246.1. HPLC (A05) t_{R}=1.78 min.

### Step 2: 2-(5-nitro-1H-pyrazol-3-yl)-1H-benzimidazole

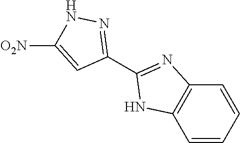 [SMILES: O=[N+]([O-])c1cc(-c2nc3ccccc3[nH]2)n[nH]1]

AcOH (100 mL) was added to N-(2-aminophenyl)-5-nitro-1H-pyrazole-3-carboxamide (3.32 g, 13.4 mmol), and the mixture was heated at 100° C. for 30 min. The mixture was filtered, and the solid was washed with water and hexanes. The solid was dried under high vacuum to provide the title compound as a solid (2.29 g, 74%). ¹H NMR (400 MHz, DMSO) δ 15.14 (br, 1H), 13.24 (br, 1H), 7.78-7.66 (m, 2H), 7.62 (s, 1H), 7.36-7.18 (m, 2H). m/z (ES), [M−H]⁻ 228.1. HPLC (A05) t_{R}=1.94 min.

### EXAMPLE B10

### Preparation of Compound O-1

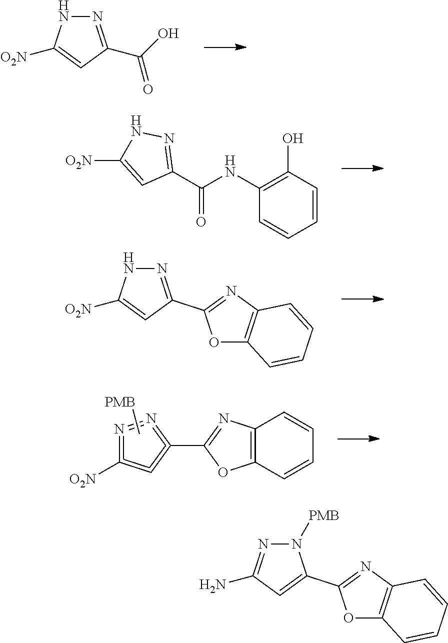

### Step 1: N-(2-hydroxyphenyl)-5-nitro-1H-pyrazole-3-carboxamide

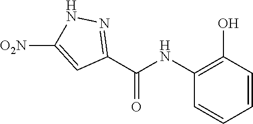 [SMILES: O=C(Nc1ccccc1O)c1cc([N+](=O)[O-])[nH]n1]

5-Nitro-1H-pyrazole-3-carboxylic acid (0.695 g, 4.42 mmol) and HATU (1.85 g, 4.86 mmol) were dissolved in DMF (22 mL). DIEA (1.52 mL, 8.87 mmol) was added, and the mixture was stirred at 23° C. for 5 min. 2-Aminophenol (0.531 g, 4.87 mmol) was added, and the mixture was heated at 50° C. for 18 h. The mixture was concentrated. Brine (10 mL) was added, and the aq. phase was extracted with EtOAc (3×30 mL). The combined organic phases were washed with brine (20 mL), dried (MgSO₄), filtered, and concentrated. The residue was diluted in DCM (75 mL) and stirred for 5 min. The mixture was filtered, and the solid was washed with hexanes and dried under high vacuum to provide the title compound as a solid (948 mg; 60% purity, 52%). ¹H NMR (500 MHz, DMSO) δ 9.90 (s, 1H), 7.77 (s, 1H), 7.61 (d, J=7.5 Hz, 1H), 7.10-7.01 (m, 1H), 6.94 (dd, J=8.1, 1.2 Hz, 1H), 6.83 (td, J=7.8, 1.3 Hz, 1H). m/z (ES⁻), [M−H]⁻ 247.1. HPLC (A05) t_{R}=1.88 min.

### Step 2: 2-(5-nitro-1H-pyrazol-3-yl)-1,3-benzoxazole

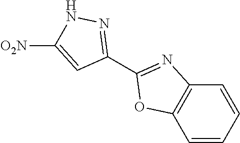 [SMILES: O=[N+]([O-])c1cc(-c2nc3ccccc3o2)n[nH]1]

Toluene (12 mL) was added to a mixture of p-TsOH—H₂O (0.480 g, 2.52 mmol) and N-(2-hydroxyphenyl)-5-nitro-1H-pyrazole-3-carboxamide (0.948 g, 60% pure, 2.29 mmol). The mixture was heated at 110° C. for 18 h. The mixture was concentrated. Sat. NaHCO₃ (20 mL) was added, and the aq. phase was extracted with EtOAc (3×30 mL). The combined organic phases were washed with brine (20 mL), dried (MgSO₄), filtered, and concentrated. The residue was diluted in a 1:1 mixture of DCM:hexanes (10 mL) and stirred for 5 min. The mixture was filtered, and the solid was washed with hexanes and dried under high vacuum to provide the title compound as a solid (0.5 g; 90% pure, 85%). ¹H NMR (500 MHz, DMSO) δ 7.89 (d, J=7.6 Hz, 1H), 7.85 (d, J=8.0 Hz, 1H), 7.76 (s, 1H), 7.56-7.46 (m, 2H). m/z (ES⁻), [M−H]⁻ 229.1. HPLC (A05) t_{R}=2.12 min.

### Step 3: 2-[2- and 2-[1-[(4-methoxyphenyl)methyl]-5-nitro-pyrazol-3-yl]-1,3-benzoxazole

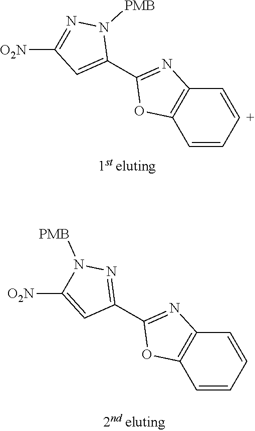

DMF (10 mL) was added to a mixture of 2-(5-nitro-1H-pyrazol-3-yl)-1,3-benzoxazole (0.5 g, 90% pure, 1.95 mmol) and Cs₂CO₃ (0.955 g, 2.93 mmol). 4-Methoxybenzyl chloride (0.345 mL, 2.54 mmol) was added dropwise, and the mixture was stirred at 50° C. for 18 h. The mixture was concentrated. Water (20 mL) was added, and the aq. phase was extracted with EtOAc (3×30 mL). The combined organic phases were washed with brine (20 mL), dried (Na₂SO₄), filtered, and concentrated. The residue was diluted in EtOAc:hexanes (1:1, 50 mL) and stirred for 5 min. The mixture was filtered, and the solid was washed with hexanes and dried under high vacuum. The solid was purified by silica gel chromatography (24 g cartridge) eluting with hexanes and DCM (0-100%) to provide two compounds. 1^{st} eluting compound as a solid (323 mg, 47%. ¹H NMR (500 MHz, CDCl₃) δ 7.88-7.83 (m, 1H), 7.64-7.60 (m, 1H), 7.58 (s, 1H), 7.49-7.42 (m, 4H), 6.85-6.81 (m, 2H), 6.08 (s, 2H), 3.75 (s, 3H). m/z (ES+), [M+Na]⁺ 373.4. HPLC (A05) t_{R}=2.57; and 2^{nd} eluting compound: 2-[2-[(4-methoxyphenyl)methyl]-5-nitro-pyrazol-3-yl]-1,3-benzoxazole as a solid (98 mg, 14%). ¹H NMR (500 MHz, CDCl₃) δ 7.85-7.79 (m, 1H), 7.74 (s, 1H), 7.67-7.60 (m, 1H), 7.45-7.38 (m, 2H), 7.38-7.33 (m, 2H), 6.88-6.83 (m, 2H), 5.87 (s, 2H), 3.78 (s, 3H). m/z (ES⁺), [M+Na]⁺ 373.1. HPLC (A05) t_{R}=2.52 min.

### Step 4: 5-(1,3-benzoxazol-2-yl)-1-[(4-methoxyphenyl)methyl]pyrazol-3-amine

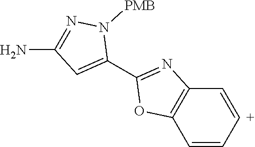

2-[2-[(4-Methoxyphenyl)methyl]-5-nitro-pyrazol-3-yl]-1,3-benzoxazole (203 mg, 0.579 mmol) was dissolved in AcOH (10 mL). Iron powder (324 mg, 5.79 mmol) was added, and the mixture was stirred at 23° C. for 4 h. The mixture was filtered through a pad of Celite, washing with EtOAc. The filtrate was concentrated. Sat. NaHCO₃ (20 mL) and Rochelle's salt (20 mL) were added. The aq. phase was extracted with EtOAc (3×30 mL). The combined organic phases were washed with sat. NaHCO₃ (30 mL), Rochelle's salt (30 mL), and brine (30 mL). The organic phases were dried (MgSO₄), filtered, and concentrated. The residue was purified by silica gel chromatography (4 g cartridge) eluting with DCM and MeOH (0-10%) to provide the title compound as a solid (139 mg, 75%). ¹H NMR (500 MHz, CDCl₃) δ 7.78-7.74 (m, 1H), 7.57-7.53 (m, 1H), 7.40-7.33 (m, 2H), 7.33-7.28 (m, 2H), 6.84-6.79 (m, 2H), 6.37 (s, 1H), 5.80 (s, 2H), 3.75 (s, 3H). m/z (ES⁺), [M+H]⁺ 321.0. HPLC (A05) t_{R}=2.27 min.

### EXAMPLE B11

### Preparation of Compound P-1

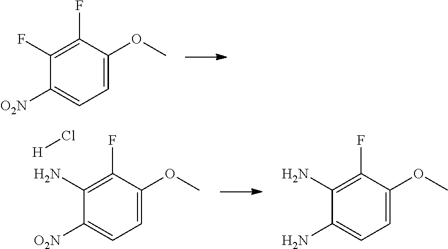

### Step 1: 2-fluoro-3-methoxy-6-nitro-aniline hydrochloride

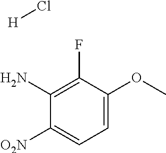

2.0 M NH₃ in MeOH (10 mL, 20.0 mmol) was added to 2,3-difluoro-1-methoxy-4-nitro-benzene (0.946 g, 5.00 mmol). The mixture was heated at 80° C. under microwave irradiation for 2 h. After cooling to 23° C., the mixture was concentrated. Sat. NaHCO₃ (20 mL) was added, and the aq. phase was extracted with Et₂O (2×30 mL). 2M HCl in ether (5 mL, 10.0 mmol) was added, and the mixture was stirred at 23° C. for 5 min. The mixture was filtered, and the solid was dried to provide the title compound as the solid (425 mg, 38%). ¹H NMR (500 MHz, DMSO) δ 7.91 (dd, J=9.8, 2.1 Hz, 1H), 7.14 (s, 2H), 6.61 (dd, J=9.8, 7.9 Hz, 1H), 3.93 (s, 3H). m/z (ES⁺), [M−H−HCl]⁻ 185.4. HPLC (A05) t_{R}=2.11 min.

### Step 2: 3-fluoro-4-methoxy-benzene-1,2-diamine

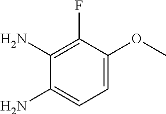

Zinc dust (0.617 g, 9.43 mmol) was added in portions at 0° C. to a mixture of 2-fluoro-3-methoxy-6-nitro-aniline hydrochloride (0.420 g, 1.89 mmol) and NH₄Cl (1.01 g, 18.9 mmol) in acetone (24 mL) and water (6 mL). The mixture was warmed to 23° C. and stirred for 2.5 h. Additional zinc dust (0.617 g, 9.43 mmol) was added, and the mixture was stirred at 23° C. for 2 h. The mixture was filtered through Celite, washing with acetone. The filtrate was concentrated. Sat. NaHCO₃ (25 mL) and EtOAc (25 mL) were added. The mixture was vigorously stirred at 23° C. for 5 min and filtered. The aq. phase was extracted with EtOAc (2×25 mL), and the combined organic phases were washed with brine (25 mL), dried (MgSO₄), filtered, and concentrated. The residue was purified by reverse phase chromatography (54 g cartridge, C-18) eluting with water ((10 mM (NH₄)(HCO₃)) and MeCN (5-100%) to provide the title compound as a solid (0.176 g, 60%). ¹H NMR (400 MHz, DMSO) δ 6.25 (dd, J=8.6, 1.9 Hz, 1H), 6.15 (t, J=8.7 Hz, 1H), 4.45 (s, 2H), 4.35 (s, 2H), 3.66 (s, 3H). ¹⁹F NMR (376 MHz, DMSO) δ −156.36 (d, J=8.6 Hz). m/z (ES⁺), [M+H]⁺ 157.5. HPLC (B05) t_{R}=1.66 min.

### EXAMPLE B12

### Preparation of Compounds A-4, A-6, A-9, A-20, A-22, A-24, A-25, A-28, A-29, A-31, A-33, A-36, A-39, A-40, A-43, A-50, A-53, A-75, A-94, A-103, A-104, A-113, A-115, A-116, A-117, A-118, A-119, A-120, A-121, A-123, A-124, A-129, A-144, and A-152

Representative synthetic scheme for the syntheses of the title compounds.

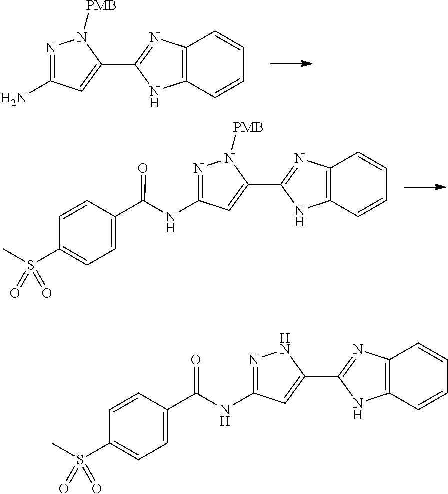

### Preparation of Compound A-4.

### Step 1: N-[5-(1H-benzimidazol-2-yl)-1-[(4-methoxyphenyl)methyl]-pyrazol-3-yl]-4-methylsulfonyl-benzamide

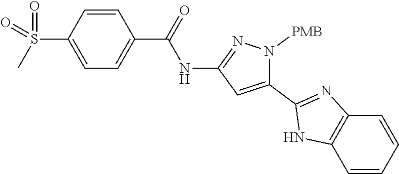

4-Methylsulfonylbenzoic acid (31.3 mg, 0.157 mmol) and HATU (59.5 mg, 0.157 mmol) were dissolved in DMF (1.30 mL) at 0° C. DIEA (0.0536 mL, 0.313 mmol) was added, and the mixture was stirred at 0° C. for 5 min. 5-(1H-benzimidazol-2-yl)-1-[(4-methoxyphenyl)methyl]pyrazol-3-amine (J-1) (50 mg, 0.157 mmol) was added. The mixture was warmed to 23° C. and stirred for 18 h. The mixture was concentrated. Water (10 mL) was added, and the aq. phase was extracted with EtOAc (3×20 mL). The combined organic phases were washed with NaHCO₃ (3×20 mL) and brine (20 mL), dried (MgSO₄), filtered, and concentrated. The product was purified by silica gel chromatography (12 g cartridge) eluting with DCM and MeOH (0-5%) to provide the title compound as a solid (50 mg; 64%). ¹H NMR (500 MHz, DMSO) δ 13.16 (s, 1H), 11.43 (s, 1H), 8.24 (d, J=8.6 Hz, 2H), 8.05 (d, J=8.5 Hz, 2H), 7.73 (d, J=8.1 Hz, 1H), 7.60 (s, 1H), 7.55 (d, J=7.7 Hz, 1H), 7.32-7.21 (m, 4H), 6.88-6.84 (m, 2H), 6.05 (s, 2H), 3.69 (s, 3H), 3.29 (s, 3H). m/z (ES+), [M+H]⁺ 502.2. HPLC (A05) t_{R}=2.62 min.

### Step 2: N-[5-(1H-benzimidazol-2-yl)-1H-pyrazol-3-yl]-4-methylsulfonyl-benzamide

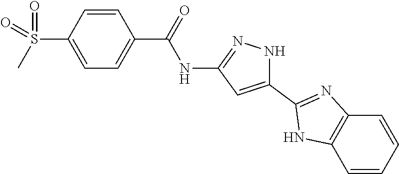 [SMILES: CS(=O)(=O)c1ccc(C(=O)Nc2cc(-c3nc4ccccc4[nH]3)[nH]n2)cc1]

N-[5-(1H-Benzimidazol-2-yl)-1-[(4-methoxyphenyl)methyl]pyrazol-3-yl]-4-methylsulfonyl-benzamide (50 mg, 0.0997 mmol) was dissolved in DCM (2 mL) at 23° C. TFA (1 mL) was added, and the solution was stirred at 23° C. for 2 h. The mixture was concentrated. Sat. NaHCO₃ (15 mL) was added, and the mixture was filtered. The solid was washed with water (10 mL), MeCN (10 mL), EtOAc (10 mL), and hexanes (10 mL) to provide the title compound as a solid (26.7 mg, 70%). ¹H NMR (500 MHz, DMSO) δ 13.57 (s, 1H), 13.05 (s, 1H), 11.33 (s, 1H), 8.25 (d, J=8.4 Hz, 2H), 8.07 (d, J=8.1 Hz, 2H), 7.68 (d, J=7.7 Hz, 1H), 7.54 (d, J=8.0 Hz, 1H), 7.47 (s, 1H), 7.29-7.19 (m, 2H), 3.30 (s, 3H). m/z (ES⁺), [M+H]⁺ 382.2. HPLC (A05) t_{R}=2.13 min.

### Preparation of Compound A-6.

### Step 1: N-[5-(1,3-benzoxazol-2-yl)-1-[(4-methoxyphenyl)methyl]pyrazol-3-yl]-3-chloro-4-methoxy-benzamide

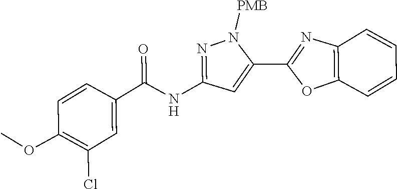

3-Chloro-4-methoxy-benzoic acid (73 mg, 0.391 mmol) and HATU (165 mg, 0.434 mmol) were dissolved in DMF (2.5 mL) at 0° C. DIEA (149 μL, 0.868 mmol) was added, and the mixture was stirred at 0° C. for 5 min. 5-(1,3-Benzoxazol-2-yl)-1-[(4-methoxyphenyl)methyl]pyrazol-3-amine (0-1) (139 mg, 0.434 mmol) was added. The mixture was stirred at 0° C. for 1 h, warmed to 23° C., and stirred for 18 h. The mixture was concentrated. Water (10 mL) was added, and the aq. phase was extracted with EtOAc (3×30 mL). The combined organic phases were washed with brine (20 mL), dried (MgSO₄), filtered, and concentrated. The product was purified by silica gel chromatography (12 g cartridge) eluting with hexanes and EtOAc (0-100%) to provide the title compound as a solid (31.1 mg; 46%). ¹H NMR (500 MHz, CDCl₃) δ 8.38 (s, 1H), 7.95 (d, J=2.2 Hz, 1H), 7.82-7.75 (m, 2H), 7.62 (s, 1H), 7.62-7.56 (m, 1H), 7.45-7.36 (m, 2H), 7.35-7.30 (m, 2H), 7.00 (d, J=8.6 Hz, 1H), 6.86-6.80 (m, 2H), 5.94 (s, 2H), 3.97 (s, 3H), 3.76 (s, 3H). m/z (ES⁺), [M+H]⁺ 489.0. HPLC (A05) t_{R}=2.66 min.

### Step 2: N-[5-(1,3-benzoxazol-2-yl)-1H-pyrazol-3-yl]-3-chloro-4-methoxy-benzamide

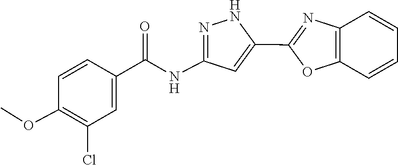 [SMILES: COc1ccc(C(=O)Nc2cc(-c3nc4ccccc4o3)[nH]n2)cc1Cl]

N-[5-(1,3-Benzoxazol-2-yl)-1-[(4-methoxyphenyl)methyl]pyrazol-3-yl]-3-chloro-4-methoxy-benzamide (105 mg, 0.215 mmol) was dissolved in DCM (2 mL). TFA (2 mL) was added, and the mixture was stirred at 23° C. for 3 h. The mixture was concentrated. The residue was diluted in sat. NaHCO₃ (10 mL) and stirred for 5 min. The mixture was filtered, and the solid was washed with water, MeCN and hexanes, and dried under high vacuum to provide a 2:1 mixture of pyrazole tautomers of the title compound as a solid (46 mg, 58%). ¹H NMR (500 MHz, DMSO) δ 14.00 (s, 1H) [13.38 (s, 1H)], 11.11 (s, 1H) [11.18 (s, 1H)], 8.27-7.96 (m, 2H) [8.27-7.96 (m, 2H)], 7.93-7.67 (m, 2H) [7.93-7.67 (m, 2H)], 7.56-7.20 (m, 3H) [7.56-7.20 (m, 3H)], 7.38 (s, 1H) [6.78 (s, 1H)], 3.95 (s, 3H) [3.95 (s, 3H)]. m/z (ES⁺), [M−H]⁻ 367.1. HPLC (A05) t_{R}=2.25 min.

### Preparation of Compound A-9.

### Step 1: N-[5-(1H-benzimidazol-2-yl)-1-[(4-methoxyphenyl)methyl]-pyrazol-3-yl]-4-cyano-benzamide

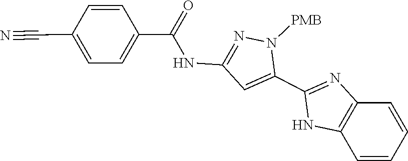

4-Cyanobenzoic acid (12.9 mg, 0.0877 mmol) and HATU (33.3 mg, 0.0877 mmol) were dissolved in DMF (1 mL) at 23° C. DIEA (30.0 μL, 0.175 mmol) was added, and the mixture was stirred at 23° C. for 5 min. 5-(1H-benzimidazol-2-yl)-1-[(4-methoxyphenyl)methyl]pyrazol-3-amine (J-1) (28 mg, 0.0877 mmol) was added, and the mixture was stirred for 18 h. The mixture was concentrated. Water (10 mL) was added, and the aq. phase was extracted with EtOAc (3×20 mL). The combined organic phases were washed with brine (20 mL), dried (MgSO₄), filtered, and concentrated. The product was purified by silica gel chromatography (12 g cartridge) eluting with hexanes and EtOAc (0-100%) to provide the title compound as a solid (35 mg; 89%). ¹H NMR (500 MHz, DMSO) δ 13.15 (s, 1H), 11.41 (s, 1H), 8.17 (d, J=8.6 Hz, 2H), 8.01-7.97 (m, 2H), 7.73 (d, J=8.1 Hz, 1H), 7.59 (s, 1H), 7.54 (d, J=8.3 Hz, 1H), 7.28 (t, J=7.6 Hz, 1H), 7.26-7.22 (m, 3H), 6.87-6.83 (m, 2H), 6.05 (s, 2H), 3.68 (s, 3H). m/z (ES⁺), [M+H]⁺ 450.1. HPLC (A05) t_{R}=2.39 min.

### Step 2: N-[5-(1H-benzimidazol-2-yl)-1H-pyrazol-3-yl]-4-cyano-benzamide

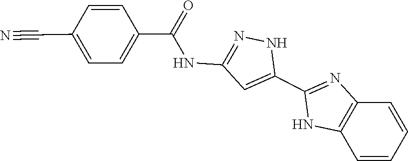 [SMILES: N#Cc1ccc(C(=O)Nc2cc(-c3nc4ccccc4[nH]3)[nH]n2)cc1]

tert-Butyl 2-[2-[(4-methoxyphenyl)methyl]-5-[(2-methoxypyrimidine-5-carbonyl)amino]pyra-zol-3-yl]benzimidazole-1-carboxylate (60 mg, 0.108 mmol) was dissolved in DCM (2 mL) at 23° C. TFA (2 mL) was added, and the mixture was stirred for 18 h at 23° C. The mixture was concentrated. Sat. NaHCO₃ (10 mL) was added, and the mixture was filtered. The solid was washed with water (10 mL), MeCN (5 mL), EtOAc (5 mL), DCM (5 mL), and hexanes (5 mL) to provide the title compound as a solid in a 5:1 mixture of pyrazole tautomers (13.8 mg, 54%). ¹H NMR (500 MHz, DMSO) δ 13.58 [13.25] (s, 1H), 13.05 [12.76] (s, 1H), [11.45] 11.32 (s, 1H), 8.18 [8.18] (d, J=8.2 Hz, 2H), [8.08] 8.01 (d, J=7.7 Hz, 2H), 7.70-7.61 [7.70-7.61] (m, 1H), 7.54 [7.54] (d, J=7.7 Hz, 1H), 7.46 [7.46] (s, 1H), 7.31-7.13 [7.31-7.13] (m, 2H). m/z (ES⁺), [M+H]⁺ 329.2. HPLC (A05) t_{R}=2.06 min.

### Preparation of Compound A-20.

### Step 1: N-[5-(1H-benzimidazol-2-yl)-1-[(4-methoxyphenyl)methyl]-pyrazol-3-yl]-4-methoxy-2,6-dimethyl-benzamide

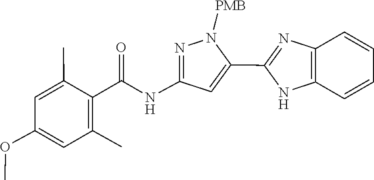

4-Methoxy-2,6-dimethyl-benzoic acid (84.6 mg, 0.470 mmol), 5-(1H-benzimidazol-2-yl)-1-[(4-methoxyphenyl)methyl]pyrazol-3-amine (J-1) (50 mg, 0.157 mmol) and 1-methylimidazole (93.6 μL, 1.17 mmol) were dissolved in MeCN (1 mL). TCFH (132 mg, 0.470 mmol) was added, and the mixture was stirred at 23° C. for 8 h. Water (25 mL) was added. The mixture was filtered, and the solid was purified by silica gel chromatography (4 g cartridge) eluting with hexanes and EtOAc (0-100%) to provide the title compound as a solid (9.9 mg, 13%). ¹H NMR (500 MHz, DMSO) δ 13.12 (s, 1H), 10.88 (s, 1H), 7.75-7.68 (m, 1H), 7.58 (s, 1H), 7.56-7.51 (m, 1H), 7.24 (d, J=8.7 Hz, 1H), 7.31-7.21 (m, J=8.7 Hz, 3H), 6.88-6.83 (m, 2H), 6.65 (s, 2H), 6.01 (s, 2H), 3.76 (s, 3H), 3.69 (s, 3H), 2.24 (s, 6H). m/z (ES⁺), [M+H]⁺ 482.2. HPLC (A05) t_{R}=2.57 min.

### Step 2: N-[5-(1H-benzimidazol-2-yl)-1H-pyrazol-3-yl]-4-methoxy-2,6-dimethyl-benzamide

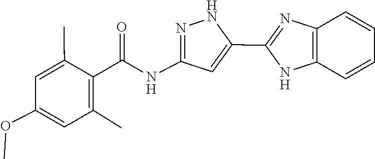 [SMILES: COc1cc(C)c(C(=O)Nc2cc(-c3nc4ccccc4[nH]3)[nH]n2)c(C)c1]

TFA (0.500 mL) was added to N-[5-(1H-benzimidazol-2-yl)-1-[(4-methoxyphenyl)methyl]-pyrazol-3-yl]-4-methoxy-2,6-dimethyl-benzamide (15 mg, 0.0311 mmol), and the mixture was heated at 70° C. for 20 min. After cooling to 23° C., the mixture was concentrated. The residue was dissolved in EtOAc (20 mL), washed with sat. NaHCO₃ (10 mL) and brine (10 mL), dried (MgSO₄), filtered, and concentrated. The residue was purified by Waters HPLC (BEH C-18 column, 30×150 mm, 5 micron) eluting with water (10 mM (NH₄)(HCO₂)) and MeCN (32-52%). The purified residue was dissolved in EtOAc (20 mL) and washed with water (10 mL). The organic phase was concentrated and dried to provide a 4:1 mixture of pyrazole tautomers of the title compound as a solid (3.08 mg, 27%). ¹H NMR (400 MHz, DMSO) δ 13.43 [13.28] (s, 1H), 13.03 [12.80] (s, 1H), 10.81 [11.20] (s, 1H), 7.73-7.59 [7.73-7.59] (m, 1H), 7.57-7.49 [7.57-7.49] (m, 1H), 7.49-7.42 [7.49-7.42] (m, 1H), 7.30-7.14 [7.30-7.14] (m, 2H), 6.80-6.63 [6.80-6.63] (m, 2H), 3.77 [3.77] (s, 3H), 2.27 [2.27] (s, 6H). m/z (ES⁺), [M+H]⁺ 362.4. HPLC (A05) t_{R}=2.14 min.

### Preparation of Compound A-22 (N-[5-(1H-benzimidazol-2-yl)-1-[(4-methoxyphenyl)methyl]pyrazol-3-yl]-3-chloro-4-methoxy-benzamide)

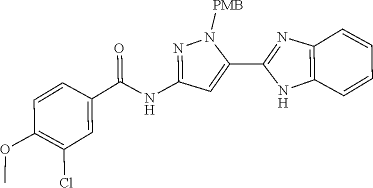

DIEA (26.8 μL, 0.157 mmol) was added to a solution of 5-(1H-benzimidazol-2-yl)-1-[(4-methoxyphenyl)methyl]pyrazol-3-amine (J-1) (25 mg, 0.0783 mmol) and 3-chloro-4-methoxy-benzoic acid (15.3 mg, 0.0822 mmol) in DMF (2 mL). HATU (32.7 mg, 0.0861 mmol) was added, and the mixture was stirred at 23° C. for 2 h. Water (20 mL) was added. The mixture was filtered, and the solid was washed with water (3×10 mL), and dissolved in EtOAc (25 mL). The organic phase was dried (MgSO₄), filtered, and concentrated. The residue was purified by silica gel chromatography (4 g, cartridge) eluting with hexanes and EtOAc (0-60%) to provide the title compound as a solid (25.6 mg, 67%). ¹H NMR (500 MHz, DMSO) δ 13.13 (s, 1H), 11.08 (s, 1H), 8.15 (d, J=2.1 Hz, 1H), 8.07 (dd, J=8.7, 2.1 Hz, 1H), 7.72 (d, J=7.0 Hz, 1H), 7.55 (s, 2H), 7.32-7.19 (m, 5H), 6.85 (d, J=8.7 Hz, 2H), 6.04 (s, 2H), 3.94 (s, 3H), 3.69 (s, 3H). m/z (ES⁺) [M+H]⁺: 489.44; HPLC (A05) t_{R}=2.62 min.

### Preparation of Compound A-24.

### Step 1: N-[5-(1H-benzimidazol-2-yl)-1-[(4-methoxyphenyl)methyl]-pyrazol-3-yl]furan-2-carboxamide

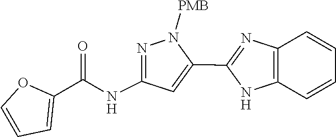

2-Furoic acid (18.4 mg, 0.164 mmol) and HATU (65.5 mg, 0.172 mmol) were dissolved in DMF (1.50 mL) at 0° C. DIEA (80.4 μL, 0.470 mmol) was added, and the mixture was stirred at 0° C. for 5 min. 5-(1H-benzimidazol-2-yl)-1-[(4-methoxyphenyl)methyl]pyrazol-3-amine (J-1) 50 mg, 0.157 mmol) was added. The mixture was warmed to 23° C. and stirred for 18 h. Water (25 mL) was added. The solid was filtered, washing with water, and dissolved in 9:1 DCM:MeOH (20 mL). The organic phase was dried (MgSO₄), filtered, and concentrated to provide the title compound as a solid (61.6 mg, 95%). ¹H NMR (400 MHz, DMSO) δ 13.14 (s, 1H), 11.00 (s, 1H), 7.92 (dd, J=1.7, 0.8 Hz, 1H), 7.73 (d, J=7.4 Hz, 1H), 7.57-7.52 (m, 2H), 7.50 (s, 1H), 7.31-7.27 (m, 1H), 7.26-7.22 (m, 3H), 6.89-6.82 (m, 2H), 6.68 (dd, J=3.5, 1.7 Hz, 1H), 6.04 (s, 2H), 3.69 (s, 3H). m/z (ES⁺), [M+H]⁺ 414.4. HPLC (A05) t_{R}=2.43 min.

### Step 2: N-[5-(1H-benzimidazol-2-yl)-1H-pyrazol-3-yl]furan-2-carboxamide

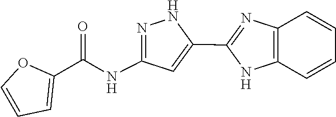 [SMILES: O=C(Nc1cc(-c2nc3ccccc3[nH]2)[nH]n1)c1ccco1]

TFA (0.500 mL) was added to a solution of N-[5-(1H-benzimidazol-2-yl)-1-[(4-methoxyphenyl)methyl]pyrazol-3-yl]furan-2-carboxamide (59.4 mg, 0.144 mmol) in DCM (0.500 mL), and the mixture was stirred at 23° C. for 2 h. The mixture was concentrated. The residue was diluted in a 1:1 mixture of EtOAc and sat. NaHCO₃ (20 mL) and stirred for 5 min. The mixture was filtered, and the solid was washed with water, EtOAc, DCM, and MeCN to provide the title compound as a solid (28.5 mg, 68%). ¹H NMR (500 MHz, CD3OD) δ 7.78 (d, J=1.0 Hz, 1H), 7.65-7.56 (m, 2H), 7.34 (d, J=3.5 Hz, 1H), 7.30-7.24 (m, 2H), 7.12-6.77 (m, 1H), 6.68 (dd, J=3.5, 1.7 Hz, 1H). m/z (ES⁺), [M+H]⁺ 293.8. HPLC (A05) t_{R}=1.92 min.

### Preparation of Compound A-25.

### Step 1: N-[5-(1H-benzimidazol-2-yl)-1-[(4-methoxyphenyl)methyl]-pyrazol-3-yl]furan-3-carboxamide

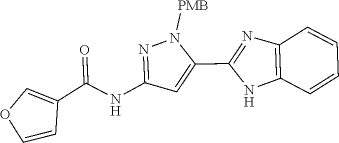

3-Furoic acid (18.4 mg, 0.164 mmol) and HATU (65.5 mg, 0.172 mmol) were dissolved in DMF (1.50 mL) at 0° C. DIEA (80.4 μL, 0.470 mmol) was added, and the mixture was stirred at 0° C. for 5 min. 5-(1H-benzimidazol-2-yl)-1-[(4-methoxyphenyl)methyl]pyrazol-3-amine (J-1) (50 mg, 0.157 mmol) was added. The mixture was warmed to 23° C. and stirred for 18 h. Water (25 mL) was added. The mixture was filtered, and the solid was washed with water, and dissolved in 9:1 DCM:MeOH (20 mL). The organic phase was dried (MgSO₄), filtered, and concentrated to provide the title compound as a solid (58.6 mg, 91%). ¹H NMR (400 MHz, DMSO) δ 13.14 (s, 1H), 10.87 (s, 1H), 8.46 (dd, J=1.5, 0.8 Hz, 1H), 7.78 (t, J=1.7 Hz, 1H), 7.75-7.71 (m, 1H), 7.57-7.53 (m, 1H), 7.51 (s, 1H), 7.31-7.27 (m, 1H), 7.26-7.21 (m, J=8.4, 4.9, 2.0 Hz, 3H), 7.06 (dd, J=1.9, 0.8 Hz, 1H), 6.88-6.84 (m, 2H), 6.03 (s, 2H), 3.69 (s, 3H). m/z (ES⁺), [M+H]⁺ 413.9. HPLC (A05) t_{R}=2.44 min.

### Step 2: N-[5-(1H-benzimidazol-2-yl)-1H-pyrazol-3-yl]furan-3-carboxamide

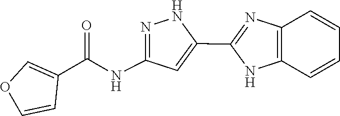 [SMILES: O=C(Nc1cc(-c2nc3ccccc3[nH]2)[nH]n1)c1ccoc1]

TFA (0.500 mL) was added to a suspension of N-[5-(1H-benzimidazol-2-yl)-1-[(4-methoxyphenyl)methyl]pyrazol-3-yl]furan-3-carboxamide (57 mg, 0.138 mmol) in DCM (0.500 mL), and the mixture was stirred at 23° C. for 2 h. The mixture was concentrated. The residue was diluted in a 1:1 mixture of EtOAc (10 mL) and sat. NaHCO₃ (10 mL) and stirred for 5 min. The mixture was filtered, and the solid was washed with water, EtOAc, DCM, and MeCN to provide the title compound as a solid (33.3 mg, 82%). ¹H NMR (500 MHz, CD₃OD) δ 8.28 (s, 1H), 7.65 (t, J=1.7 Hz, 1H), 7.63-7.55 (m, 2H), 7.30-7.23 (m, 2H), 6.97 (dd, J=1.9, 0.8 Hz, 1H), 6.71 (br, 1H). m/z (ES⁺), [M+H]⁺ 293.9. HPLC (A05) t_{R}=1.96 min.

### Preparation of Compound A-28.

### Step 1: N-[5-(1H-benzimidazol-2-yl)-1-[(4-methoxyphenyl)methyl]pyrazol-3-yl]-5-chloro-6-methoxy-pyridine-3-carboxamide

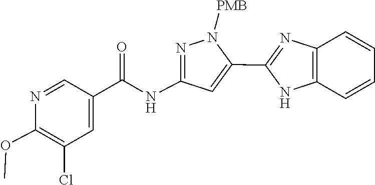

5-Chloro-6-methoxy-pyridine-3-carboxylic acid (30.8 mg, 0.164 mmol) and HATU (65.5 mg, 0.172 mmol) were dissolved in DMF (1.50 mL) at 0° C. DIEA (80.4 μL, 0.470 mmol) was added, and the mixture was stirred at 0° C. for 15 min. 5-(1H-benzimidazol-2-yl)-1-[(4-methoxyphenyl)methyl]pyrazol-3-amine (J-1) (50 mg, 0.157 mmol) was added. The mixture was warmed to 23° C. and stirred for 7 h. Water (15 mL) was added. The mixture was filtered, and the solid was washed with water (10 mL), and purified by silica gel chromatography (12 g cartridge) eluting with hexanes and EtOAc (0-100%) to provide the title compound as a solid (49.2 mg, 64%). ¹H NMR (500 MHz, DMSO) δ 13.15 (s, 1H), 10.94 (s, 1H), 8.65 (s, 1H), 8.35 (s, 1H), 7.73 (d, J=7.8 Hz, 1H), 7.55 (d, J=7.1 Hz, 1H), 7.51 (s, 1H), 7.29 (t, J=7.5 Hz, 1H), 7.27-7.22 (m, 3H), 6.89-6.84 (m, 2H), 6.04 (s, 2H), 3.70 (s, 3H), 3.60 (s, 3H). m/z (ES⁺), [M+H]⁺ 489.4. HPLC (A05) t_{R}=2.37 min.

### Step 2: N-[5-(1H-benzimidazol-2-yl)-1H-pyrazol-3-yl]-5-chloro-6-methoxy-pyridine-3-carboxamide

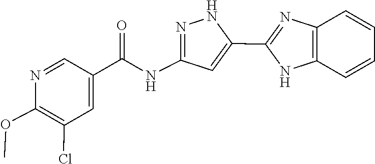 [SMILES: COc1ncc(C(=O)Nc2cc(-c3nc4ccccc4[nH]3)[nH]n2)cc1Cl]

TFA (1 mL) was added to N-[5-(1H-benzimidazol-2-yl)-1-[(4-methoxyphenyl)methyl]-pyrazol-3-yl]-5-chloro-6-methoxy-pyridine-3-carboxamide (46.7 mg, 0.0955 mmol), and the mixture was heated at 70° C. for 20 min. The mixture was concentrated. The residue was diluted in a 1:1 mixture of EtOAc and sat. NaHCO₃ (20 mL) and stirred for 5 min. The mixture was filtered, and the solid was washed with water (10 mL), and dissolved in refluxing EtOH (30 mL) at 80° C. The solution was filtered, and the filtrate was concentrated. The residue was diluted in Et₂O (15 mL). The solid was filtered and dried under high vacuum to provide the title compound as a solid (17.5 mg, 50%). ¹H NMR (500 MHz, DMSO) δ 13.52 (br, 1H), 13.04 (br, 1H), 10.88 (br, 1H), 8.65 (s, 1H), 8.36 (s, 1H), 7.72-7.62 (m, 1H), 7.58-7.49 (m, 1H), 7.44-7.34 (m, 1H), 7.29-7.18 (m, 2H), 3.62 (s, 3H). m/z (ES⁺), [M−H]⁻ 367.3. HPLC (A05) t_{R}=1.92 min.

### Preparation of Compound A-29.

### Step 1: N-[5-(1H-benzimidazol-2-yl)-1-[(4-methoxyphenyl)methyl]-pyrazol-3-yl]-3-chloro-4-(2-methoxyethoxy)benzamide

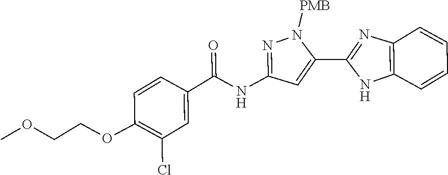

3-Chloro-4-(2-methoxyethoxy)benzoic acid (K-1) (31.5 mg, 0.136 mmol) and HATU (59.5 mg, 0.157 mmol) were dissolved in DMF (1.5 mL) at 0° C. DIEA (53.6 μL, 0.313 mmol) was added, and the mixture was stirred at 0° C. for 5 min. 5-(1H-benzimidazol-2-yl)-1-[(4-methoxyphenyl)methyl]pyrazol-3-amine (J-1) (50 mg, 0.157 mmol) was added. The mixture was warmed to 23° C. and stirred for 4 h. The mixture was concentrated. Water (10 mL) was added, and the aq. phase was extracted with EtOAc (3×20 mL). The combined organic phases were washed with brine (20 mL), dried (MgSO₄), filtered, and concentrated. The product was purified by silica gel chromatography (12 g cartridge) eluting with hexanes and EtOAc (0-100%) to provide the title compound as a solid (80 mg, 96%). ¹H NMR (400 MHz, DMSO) δ 13.13 (s, 1H), 11.07 (s, 1H), 8.15 (d, J=2.2 Hz, 1H), 8.04 (dd, J=8.7, 2.2 Hz, 1H), 7.72 (d, J=8.0 Hz, 1H), 7.54 (d, J=7.1 Hz, 2H), 7.30-7.21 (m, 5H), 6.85 (d, J=8.8 Hz, 2H), 6.03 (s, 2H), 4.30-4.27 (m, 2H), 3.74-3.70 (m, 2H), 3.68 (s, 3H), 3.34 (s, 3H).

### Step 2: N-[5-(1H-benzimidazol-2-yl)-1H-pyrazol-3-yl]-3-chloro-4-(2-methoxyethoxy)benzamide

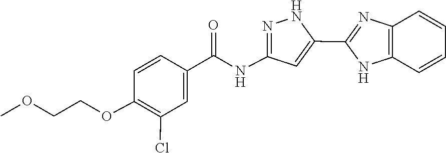 [SMILES: COCCOc1ccc(C(=O)Nc2cc(-c3nc4ccccc4[nH]3)[nH]n2)cc1Cl]

A solution of N-[5-(1H-benzimidazol-2-yl)-1-[(4-methoxyphenyl)methyl]pyrazol-3-yl]-3-chloro-4-(2-methoxyethoxy)benzamide (80 mg, 0.150 mmol) in TFA (3 mL) was heated at 70° C. for 1 h. The mixture was concentrated. Sat. NaHCO₃ (10 mL) was added, and the aq. phase was extracted with EtOAc (3×20 mL). The combined organic phases were washed with brine (20 mL), dried (MgSO₄), filtered, and concentrated. The product was purified by silica gel chromatography (25 g cartridge) eluting with DCM and MeOH (0-10%) to provide a 7:3 mixture of pyrazole tautomers of the title compound as a solid (21 mg, 34%). ¹H NMR (400 MHz,

DMSO) δ 13.49 [13.14] (s, 1H), 13.02 [12.74] (s, 1H), [11.12] 10.99 (s, 1H), 8.16 [8.12] (d, J=2.1 Hz, 1H), 8.04 [8.00] (dd, J=8.7, 2.1 Hz, 1H), 7.67 [7.62] (d, J=7.4 Hz, 1H), 7.53 [7.46] (d, J=7.2 Hz, 1H), 7.42 [7.36] (d, J=1.8 Hz, 1H), 7.33-7.11 [7.33-7.11] (m, 3H), 4.35-4.26 [4.35-4.26] (m, 2H), 3.74-3.72 [3.74-3.72] (m, 2H), 3.35 [3.35] (s, 3H). m/z (ES⁺), [M+H]⁺ 412.3. HPLC (A05) t_{R}=2.21 min.

### Preparation of Compound A-31.

### Step 1: N-[5-(1H-benzimidazol-2-yl)-1-[(4-methoxyphenyl)methyl]pyrazol-3-yl]-4-bromo-benzamide

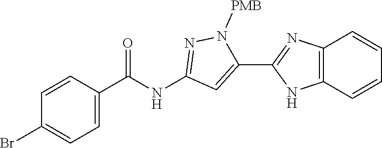

DIEA (1.91 mL, 11.0 mmol) was added to a solution of HATU (1.31 g, 3.44 mmol) and 4-bromobenzoic acid (0.661 g, 3.29 mmol) in DMF (30 mL) at 0° C. The mixture was stirred at 0° C. for 20 min. 5-(1H-benzimidazol-2-yl)-1-[(4-methoxyphenyl)methyl]pyrazol-3-amine (J-1) (1.00 g, 3.13 mmol) was added. The mixture was warmed to 23° C., stirred for 18 h, and poured into water (500 mL). The mixture was stirred at 23° C. for 10 min. The mixture was filtered, and the solid was purified by silica gel chromatography (80 g cartridge) eluting with mixtures of hexanes and EtOAc (0-100%) to provide the title compound as a solid (0.282 g, 18%). ¹H NMR (500 MHz, DMSO) δ 13.14 (s, 1H), 11.22 (s, 1H), 8.04-7.90 (m, 2H), 7.76-7.68 (m, 3H), 7.58 (s, 1H), 7.57-7.53 (m, 1H), 7.32-7.27 (m, 1H), 7.27-7.19 (m, 3H), 6.89-6.82 (m, 2H), 6.05 (s, 2H), 3.69 (s, 3H). m/z (ES⁺), [M+H]⁺ 502.5. HPLC (A05) t_{R}=2.67 min.

### Step 2: N-[5-(1H-benzimidazol-2-yl)-1H-pyrazol-3-yl]-4-bromo-benzamide

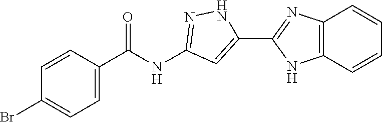 [SMILES: O=C(Nc1cc(-c2nc3ccccc3[nH]2)[nH]n1)c1ccc(Br)cc1]

A solution of N-[5-(1H-benzimidazol-2-yl)-1-[(4-methoxyphenyl)methyl]pyrazol-3-yl]-4-bromo-benzamide (34 mg, 0.0677 mmol) in TFA (2 mL) was heated to 70° C. for 1 h. The mixture was concentrated. Sat. NaHCO₃ (10 mL) was added, and the aq. phase was extracted with EtOAc (3×20 mL). The combined organic phase was washed with brine (20 mL) and concentrated. The residue was purified by reverse phase chromatography (BEH C18 30×100) eluting with water (10 mM (NH₄)(HCO₂)) and MeCN (36-56%) to provide the title compound as a solid (12 mg, 46%). ¹H NMR (400 MHz, DMSO) δ 11.14 (s, 1H), 8.34 (s, 1H), 8.01-7.96 (m, 2H), 7.75 (d, J=8.2 Hz, 2H), 7.59 (d, J=42.8 Hz, 2H), 7.22 (d, J=5.8 Hz, 2H). m/z (ES⁺), [M+H]⁺ 384.2. HPLC (A05) t_{R}=2.24 min.

### Preparation of Compound A-33.

### Step 1: N-[5-(1H-benzimidazol-2-yl)-1-[(4-methoxyphenyl)methyl]pyrazol-3-yl]-3-chloro-4-(2-morpholinoethoxy)benzamide

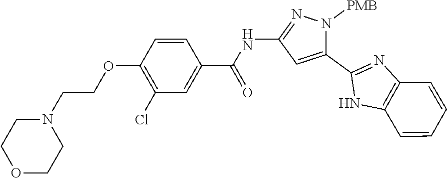

3-Chloro-4-(2-morpholinoethoxy)benzoic acid hydrochloride (K-14) (151 mg, 0.470 mmol) and HATU (0.179 g, 0.470 mmol) were dissolved in DMF (4.50 mL) at 0° C. DIEA (0.241 mL, 1.41 mmol) was added, and the mixture was stirred at 0° C. for 5 min. 5-(1H-benzimidazol-2-yl)-1-[(4-methoxyphenyl)methyl]pyrazol-3-amine (J-1) (150 mg, 0.470 mmol) was added. The mixture was warmed to 23° C., stirred for 18 h, and concentrated. Water (30 mL) was added, and the aq. phase was extracted with EtOAc (3×40 mL). The combined organic phases were washed with brine (40 mL), dried (MgSO₄), filtered, and concentrated. The product was purified by silica gel chromatography (25 g cartridge) eluting with DCM and MeOH (0-10%) to provide the title compound as a solid (86 mg, 31%). ¹H NMR (400 MHz, DMSO) δ 13.13 (s, 1H), 11.07 (s, 1H), 8.15 (d, J=2.3 Hz, 1H), 8.04 (dd, J=8.7, 2.2 Hz, 1H), 7.72 (d, J=7.8 Hz, 1H), 7.55 (s, 1H), 7.31-7.20 (m, 6H), 6.88-6.84 (m, 2H), 6.04 (s, 2H), 4.27 (t, J=5.7 Hz, 2H), 3.69 (s, 3H), 3.60-3.55 (m, 4H), 2.76 (t, J=5.8 Hz, 2H), 2.54-2.51 (m, 4H). m/z (ES⁺), [M+H]⁺ 588.7. HPLC (A05) t_{R}=2.25 min.

### Step 2: N-[5-(1H-benzimidazol-2-yl)-1H-pyrazol-3-yl]-3-chloro-4-(2-morpholinoethoxy)benzamide

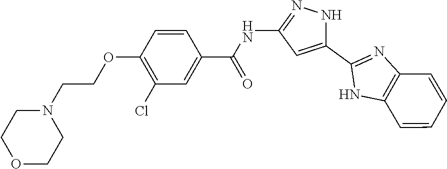 [SMILES: O=C(Nc1cc(-c2nc3ccccc3[nH]2)[nH]n1)c1ccc(OCCN2CCOCC2)c(Cl)c1]

A solution of N-[5-(1H-benzimidazol-2-yl)-1-[(4-methoxyphenyl)methyl]pyrazol-3-yl]-3-chloro-4-(2-morpholinoethoxy)benzamide (86 mg, 0.146 mmol) in TFA (5 mL) was heated to 70° C. for 1 h. The mixture was concentrated. Sat. NaHCO₃ (10 mL) was added, and the aq. phase was extracted with EtOAc (3×20 mL). The combined organic phases were washed with brine (20 mL), dried (MgSO₄), filtered, and concentrated. The residue was purified by silica gel chromatography (25 g) eluting with DCM and MeOH (0-10%), followed by Waters HPLC (BEH C18 30×150) eluting with water ((10 mM (NH₄)(HCO₃)) and MeCN (38-48%) to provide the title compound as a solid (12 mg, 17%). ¹H NMR (400 MHz, DMSO) δ 8.15 (d, J=2.3 Hz, 1H), 8.04 (d, J=9.8 Hz, 1H), 7.71-7.58 (m, 1H), 7.57-7.44 (m, 1H), 7.31 (d, J=6.6 Hz, 1H), 7.26-7.15 (m, 2H), 4.29 (t, J=5.5 Hz, 2H), 3.60-3.56 (m, 4H), 2.77 (t, J=5.7 Hz, 2H), 2.53-2.51 (m, 4H). m/z (ES⁺), [M+H]⁺ 467.4. HPLC (A05) t_{R}=1.89 min.

### Preparation of Compound A-36.

### Step 1: N-[5-(1H-benzimidazol-2-yl)-1-[(4-methoxyphenyl)methyl]-pyrazol-3-yl]-4-(4-hydroxy-1-piperidyl)benzamide

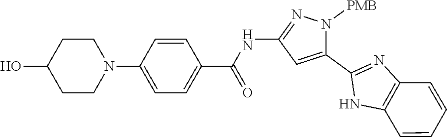

DIEA (0.191 mL, 1.10 mmol) was added to a solution of HATU (131 mg, 0.344 mmol) and 4-(4-hydroxy-1-piperidyl)benzoic acid (K-20) (72.7 mg, 0.329 mmol) in DMF (2.50 mL) at 0° C. The mixture was stirred at 0° C. for 30 min. 5-(1H-benzimidazol-2-yl)-1-[(4-methoxyphenyl)methyl]pyrazol-3-amine (J-1) (100 mg, 0.313 mmol) was added. The mixture was warmed to 23° C., stirred for 42 h, and poured into water (30 mL). The mixture was filtered, and the solid was washed with water, and purified by silica gel chromatography (25 g cartridge) eluting with DCM and MeOH (0-10%) to provide the title compound as a solid (64.4 mg; 39%). ¹H NMR (500 MHz, DMSO) δ 13.11 (s, 1H), 10.72 (s, 1H), 7.97-7.89 (m, 2H), 7.76-7.67 (m, 1H), 7.55 (s, 1H), 7.57-7.54 (m, 1H), 7.31-7.19 (m, 4H), 7.00-6.92 (m, 2H), 6.89-6.82 (m, 2H), 6.03 (s, 2H), 4.70 (d, J=4.3 Hz, 1H), 3.75-3.65 (m, 2H), 3.69 (s, 3H), 3.18 (d, J=5.2 Hz, 1H), 3.07-2.95 (m, 2H), 1.86-1.74 (m, 2H), 1.49-1.37 (m, 2H). m/z (ES⁺), [M+H]⁺ 222.2. HPLC (A05) t_{R}=2.01 min.

### Step 2: N-[5-(1H-benzimidazol-2-yl)-1H-pyrazol-3-yl]-4-(4-hydroxy-1-piperidyl)-benzamide

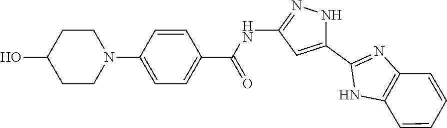 [SMILES: O=C(Nc1cc(-c2nc3ccccc3[nH]2)[nH]n1)c1ccc(N2CCC(O)CC2)cc1]

TFA (1 mL) was added to N-[5-(1H-benzimidazol-2-yl)-1-[(4-methoxyphenyl)methyl]-pyrazol-3-yl]-4-(4-hydroxy-1-piperidyl)benzamide (61.7 mg, 0.118 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 5 min. Water (25 mL) was added. The mixture was filtered, and the solid was washed with water (25 mL), acetone (25 mL), and DCM (25 mL) and dried under high vacuum to provide a 3:2 mixture of pyrazole tautomers of the title compound as a solid (34.2 mg, 72%). ¹H NMR (500 MHz, DMSO) δ 13.41 [13.06] (s, 1H), 13.01 [12.72] (s, 1H), 10.63 [10.85] (s, 1H), 7.98-7.86 [7.98-7.86] (m, 2H), 7.71-7.65 [7.65-7.59] (m, 1H), 7.57-7.51 [7.49-7.44] (m, 1H), 7.42 [6.63] (s, 1H), 7.28-7.21 [7.21-7.13] (m, 2H), 7.08-7.02 [7.02-6.95] (m, 2H), 4.75-4.68 [4.75-4.68] (m, 1H), 3.78-3.65 [3.78-3.65] (m, 3H), 3.12-2.97 [3.12-2.97] (m, 2H), 1.88-1.77 [1.88-1.77] (m, 2H), 1.50-1.39 [1.50-1.39] (m, 2H). m/z (ES⁺), [M+H]⁺ 403.5. HPLC (A05) t_{R}=1.97 min.

### Preparation of Compound A-39.

### Step 1: N-[5-(1H-benzimidazol-2-yl)-1-[(4-methoxyphenyl)methyl]pyrazol-3-yl]-3-chloro-4-(2-methoxyethylamino)benzamide

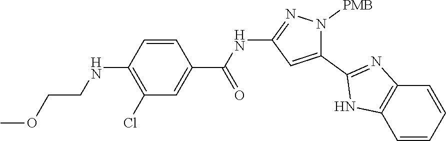

DIEA (134 μL, 0.767 mmol) was added to a solution of TBTU (77.4 mg, 0.241 mmol) and 4-(2-methoxyethylamino)benzoic acid (K-19) (47.1 mg, 0.241 mmol) in DMF (2.10 mL) at 0° C. The mixture was stirred at 0° C. for 20 min. 5-(1H-benzimidazol-2-yl)-1-[(4-methoxyphenyl)methyl]pyrazol-3-amine (J-1) (70 mg, 0.219 mmol) was added. The mixture was warmed to 23° C. and stirred for 4 days. The mixture was concentrated. Water (30 mL) was added, and the aq. phase was extracted with EtOAc (3×30 mL). The combined organic phases were washed with brine (40 mL), dried (MgSO₄), filtered, and concentrated. The residue was purified by silica gel chromatography (12 g cartridge) eluting with DCM and MeOH (0-10%) to provide the title compound as a solid (38 mg, 35%). ¹H NMR (400 MHz, DMSO) δ 13.12 (s, 1H), 10.80 (s, 1H), 8.04 (d, J=2.1 Hz, 1H), 7.92 (dd, J=8.6, 2.1 Hz, 1H), 7.72 (d, J=7.8 Hz, 1H), 7.55-7.52 (m, 2H), 7.28-7.21 (m, 4H), 6.87-6.83 (m, 2H), 6.82 (d, J=8.9 Hz, 1H), 6.02 (s, 2H), 5.82 (t, J=5.7 Hz, 1H), 3.68 (s, 3H), 3.53-3.51 (m, 2H), 3.40 (q, J=5.7 Hz, 2H), 3.29 (s, 3H). m/z (ES⁺), [M+H]⁺ 532.6. HPLC (A05) t_{R}=2.63 min.

### Step 2: N-[5-(1H-benzimidazol-2-yl)-1H-pyrazol-3-yl]-3-chloro-4-(2-methoxyethylamino)benzamide

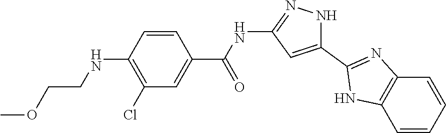 [SMILES: COCCNc1ccc(C(=O)Nc2cc(-c3nc4ccccc4[nH]3)[nH]n2)cc1Cl]

A solution of N-[5-(1H-benzimidazol-2-yl)-1-[(4-methoxyphenyl)methyl]pyrazol-3-yl]-3-chloro-4-(2-methoxyethylamino)benzamide (38 mg, 0.0716 mmol) in TFA (3 mL) was stirred at 23° C. for 1 h. The mixture was concentrated. Sat. NaHCO₃ (10 mL) was added. The mixture was filtered, and the solid was washed with water (20 mL), and purified by silica gel chromatography (4 g) eluting with DCM and MeOH (0-40%) to provide the title compound as a solid and a 3:1 mixture of the pyrazole tautomers (13.1 mg, 45%). ¹H NMR (400 MHz, DMSO) δ 13.43 [13.13] (s br, 1H), 13.02 [12.72] (s br, 1H), [10.93] 10.71 (s br, 1H), 8.03 [8.03] (s, 1H), 7.94-7.85 [7.94-7.85] (m, 1H), 7.76-7.30 [7.76-7.30] (m, 3H), 7.27-7.13 [7.27-7.13] (m, 2H), 6.93-6.78 [6.93-6.78] (m, 1H), 5.99-5.82 [5.99-5.82] (m, 1H), 3.54 [3.54] (t, J=5.6 Hz, 2H), 3.42 [3.42] (dd, J=11.1, 5.4 Hz, 2H), 3.30 [3.30] (s, 3H). m/z (ES⁺), [M+H]⁺ 411.4. HPLC (A05) t_{R}=2.22 min.

### Preparation of Compound A-40.

### Step 1: N-[5-(1H-benzimidazol-2-yl)-1-[(4-methoxyphenyl)-methyl]pyrazol-3-yl]-4-[(3R)-3-hydroxypyrrolidin-1-yl]benzamide

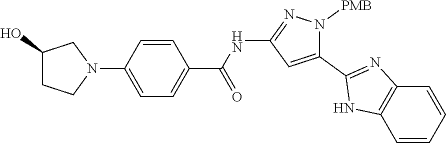

DIEA (0.132 mL, 0.756 mmol) was added to a solution of HATU (90.4 mg, 0.238 mmol) and 4-[(3R)-3-hydroxypyrrolidin-1-yl]benzoic acid (K-17) (47 mg, 0.227 mmol) in DMF (2 mL) at 0° C. The mixture was stirred at 0° C. for 30 min. 5-(1H-benzimidazol-2-yl)-1-[(4-methoxyphenyl)methyl]pyrazol-3-amine (J-1) (69 mg, 0.216 mmol) was added. The mixture was warmed to 23° C., stirred for 4 days, and poured into water (30 mL). The mixture was filtered, and the solid was washed with water, and purified by silica gel chromatography (12 g cartridge) eluting with DCM and MeOH (0-5%) to provide the title compound as a solid (34.8 mg; 32%). ¹H NMR (500 MHz, DMSO) δ 13.11 (s, 1H), 10.62 (s, 1H), 7.95 (d, J=8.9 Hz, 2H), 7.72 (d, J=8.1 Hz, 1H), 7.55 (s, 1H), 7.54 (d, J=6.7 Hz, 1H), 7.28 (t, J=6.9 Hz, 1H), 7.25-7.20 (m, 3H), 6.89-6.82 (m, 2H), 6.55 (d, J=9.0 Hz, 2H), 6.03 (s, 2H), 5.00 (d, J=3.6 Hz, 1H), 4.47-4.38 (m, 1H), 3.69 (s, 3H), 3.47 (dd, J=10.3, 4.8 Hz, 1H), 3.43-3.35 (m, 2H), 3.19-3.15 (m, 1H), 2.11-2.01 (m, 1H), 1.97-1.87 (m, 1H). m/z (ES⁺), [M+H]⁺ 509.6. HPLC (A05) t_{R}=2.39 min.

### Step 2: N-[5-(1H-benzimidazol-2-yl)-1H-pyrazol-3-yl]-4-[(3R)-3-hydroxypyrrolidin-1-yl]benzamide

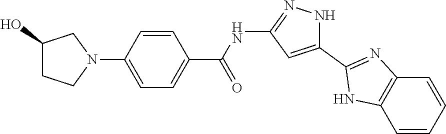 [SMILES: O=C(Nc1cc(-c2nc3ccccc3[nH]2)[nH]n1)c1ccc(N2CC[C@@H](O)C2)cc1]

TFA (1 mL) was added to N-[5-(1H-benzimidazol-2-yl)-1-[(4-methoxyphenyl)methyl]pyrazol-3-yl]-4-[(3R)-3-hydroxypyrrolidin-1-yl]benzamide (32 mg, 0.0629 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 5 min. Water (25 mL) was added. The mixture was filtered, and the solid was washed with water (25 mL), acetone (25 mL), DCM (25 mL), and acetone (25 mL) and dried under high vacuum to provide a 3:2 mixture of pyrazole tautomers of the title compound as a solid (19.9 mg, 81%). ¹H NMR (500 MHz, DMSO) δ 13.38 [13.00] (br, 1H), 13.00 [12.72] (br, 1H), 10.53 [10.76] (br, 1H), 8.02-7.86 [8.02-7.86] (m, 2H), 7.71-7.59 [7.71-7.59] (m, 1H), 7.58-7.44 [7.58-7.44] (m, 1H), 7.41 [6.60] (br, 1H), 7.30-7.11 [7.30-7.11] (m, 2H), 6.73-6.48 [6.73-6.48] (m, 2H), 5.02 [5.02] (br, 1H), 4.44 [4.44] (br, 1H), 3.54-3.46 [3.54-3.46] (m, 1H), 3.46-3.36 [3.46-3.36] (m, 2H), 3.25-3.13 [3.25-3.13] (m, 1H), 2.14-2.02 [2.14-2.02] (m, 1H), 1.99-1.90 [1.99-1.90] (m, 1H). m/z (ES+), [M+H]+ 389.5. HPLC (A05) t_{R}=1.98 min.

### Preparation of Compound A-43.

### Step 1: N-[5-(1H-benzimidazol-2-yl)-1-[(4-methoxyphenyl)-methyl]pyrazol-3-yl]-4-[(3S)-3-hydroxypyrrolidin-1-yl]benzamide

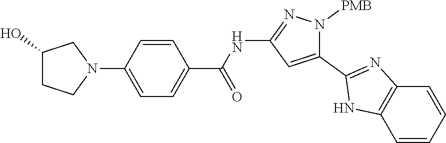

4-[(3S)-3-Hydroxypyrrolidin-1-yl]benzoic acid (K-18) (59 mg, 0.285 mmol) and HATU (83.3 mg, 0.219 mmol) were dissolved in DMF (2 mL) at 0° C. DIEA (76.4 μL, 0.438 mmol) was added, and the mixture was stirred at 23° C. for 5 min. 5-(1H-benzimidazol-2-yl)-1-[(4-methoxyphenyl)methyl]pyrazol-3-amine (J-1) (70 mg, 0.219 mmol) was added, and the mixture was stirred for 18 h. The mixture was concentrated. Water (30 mL) was added, and the aq. phase was extracted with EtOAc (3×30 mL). The combined organic phases were washed with brine (40 mL), dried (MgSO₄), filtered, and concentrated. The residue was purified by silica gel chromatography (12 g cartridge) eluting with DCM and MeOH (0-10%) to provide the title compound as a solid (71 mg, 64%). ¹H NMR (400 MHz, DMSO) δ 13.10 (s, 1H), 10.61 (s, 1H), 7.94 (d, J=9.0 Hz, 2H), 7.71 (d, J=7.5 Hz, 1H), 7.55-7.50 (m, 2H), 7.30-7.20 (m, 4H), 6.87-6.83 (m, 2H), 6.54 (d, J=9.0 Hz, 2H), 6.02 (s, 2H), 4.99 (d, J=3.7 Hz, 1H), 4.41 (s, 1H), 3.68 (s, 3H), 3.46 (dd, J=10.7, 4.9 Hz, 1H), 3.41-3.35 (m, 2H), 3.16 (d, J=9.6 Hz, 1H), 2.07-1.99 (m, 1H), 1.96-1.88 (m, 1H). m/z (ES⁺), [M+H]⁺ 509.9. HPLC (A05) t_{R}=2.39 min.

### Step 2: N-[5-(1H-benzimidazol-2-yl)-1H-pyrazol-3-yl]-4-[(3S)-3-hydroxy-pyrrolidin-1-yl]benzamide

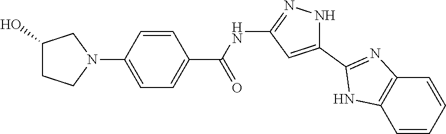 [SMILES: O=C(Nc1cc(-c2nc3ccccc3[nH]2)[nH]n1)c1ccc(N2CC[C@H](O)C2)cc1]

A solution of N-[5-(1H-benzimidazol-2-yl)-1-[(4-methoxyphenyl)methyl]pyrazol-3-yl]-4-[(3S)-3-hydroxypyrrolidin-1-yl]benzamide (71 mg, 0.140 mmol) in TFA (4 mL) was stirred at 23° C. for 1 h. The mixture was concentrated. The residue was diluted in sat. NaHCO₃ (10 mL) and stirred for 10 min. The mixture was filtered, and the solid was purified by silica gel chromatography (4 g cartridge) eluting with DCM and MeOH (0-40%) and by reverse phase chromatography (BEH C18 30×150) eluting with water (10 mM (NH₄)(HCO₂)) and MeCN (26-46%). The purified residue was diluted in sat. NaHCO₃ (10 mL) and stirred for 10 min. The mixture was filtered, and the solid was dried under high vacuum to provide the title compound as a solid in a 5:3 ratio of both tautomers (6 mg, 11%). ¹H NMR (500 MHz, DMSO) δ 13.38 [13.03] (s, 1H), 13.00 [12.71] (s, 1H), [10.76] 10.52 (s, 1H), 7.95 [7.91] (d, J=8.8 Hz, 2H), 7.67 [7.62] (d, J=8.0 Hz, 1H), 7.53 [7.46] (d, J=7.4 Hz, 1H), 7.41 [7.41] (s, 1H), 7.28-7.13 [7.28-7.13] (m, 2H), 6.63 [6.63] (d, J=9.8 Hz, 1H), 6.56 [6.56] (d, J=8.8 Hz, 1H), 5.02 [5.00] (d, J=4.0 Hz, 1H), 4.43 [4.43] (s, 1H), 3.52-3.46 [3.52-3.46] (m, 1H), 3.40 [3.40] (ddd, J=17.8, 12.6, 7.7 Hz, 2H), 3.19 [3.19] (t, J=10.4 Hz, 1H), 2.11-2.02 [2.11-2.02] (m, 1H), 1.97-1.91 [1.97-1.91] (m, 1H). m/z (ES⁺), [M+H]⁺ 389.4. HPLC (A05) t_{R}=1.98 min.

### Preparation of Compound A-50.

### Step 1: N-[5-(1H-benzimidazol-2-yl)-1-[(4-methoxyphenyl)methyl]pyrazol-3-yl]-3-chloro-4-(2-hydroxyethoxy)benzamide

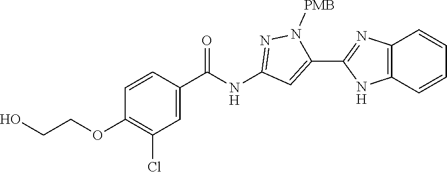

3-Chloro-4-(2-hydroxyethoxy)benzoic acid (K-8) (420 mg, 1.94 mmol) and HATU (0.737 g, 1.94 mmol) were dissolved in DMF (12 mL) at 0° C. DIEA (0.664 mL, 3.88 mmol) was added, and the mixture was stirred at 0° C. for 5 min. 5-(1H-benzimidazol-2-yl)-1-[(4-methoxyphenyl)-methyl]pyrazol-3-amine (J-1) (619 mg, 1.94 mmol) was added. The mixture was warmed to 23° C. and stirred for 18 h. The mixture was concentrated. Water (50 mL) was added, and the aq. phase was extracted with EtOAc (3×50 mL). The combined organic phases were washed with brine (60 mL), dried (MgSO₄), filtered, and concentrated. The product was purified by silica gel chromatography (40 g cartridge) eluting with DCM and MeOH (0-10%) to provide the title compound as a solid (621 mg, 61%). ¹H NMR (400 MHz, DMSO) δ 13.13 (s, 1H), 11.07 (s, 1H), 8.15 (d, J=2.3 Hz, 1H), 8.03 (dd, J=8.7, 2.3 Hz, 1H), 7.72 (d, J=7.5 Hz, 1H), 7.56-7.52 (m, 2H), 7.31-7.20 (m, 5H), 6.88-6.83 (m, 2H), 6.03 (s, 2H), 4.92 (t, J=5.2 Hz, 1H), 4.20-4.16 (m, 2H), 3.77 (dd, J=10.0, 5.3 Hz, 2H), 3.69 (s, 3H). m/z (ES⁺), [M+H]⁺ 518.6. HPLC (A05) t_{R}=2.43 min.

### Step 2: N-[5-(1H-benzimidazol-2-yl)-1H-pyrazol-3-yl]-3-chloro-4-(2-hydroxy-ethoxy)benzamide

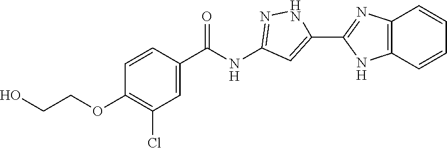 [SMILES: O=C(Nc1cc(-c2nc3ccccc3[nH]2)[nH]n1)c1ccc(OCCO)c(Cl)c1]

A solution of N-[5-(1H-benzimidazol-2-yl)-1-[(4-methoxyphenyl)methyl]pyrazol-3-yl]-3-chloro-4-(2-hydroxyethoxy)benzamide (100 mg, 0.193 mmol) in TFA (3 mL) was stirred at 23° C. for 1 h. The mixture was concentrated. The residue was dissolved in DMSO (0.500 mL) and NEt₃ (0.500 mL), and the mixture was stirred at 23° C. for 30 min. Water (10 mL) was added. The mixture was filtered, and the solid was purified by silica gel chromatography (12 g cartridge) eluting with DCM and MeOH (0-30%) to provide the title compound as a solid in a 4:1 mixture of both tautomers (21 mg, 27%). ¹H NMR (500 MHz, DMSO) δ 13.50 [13.24] (s, 1H), 13.04 [12.73] (s, 1H), [11.19] 10.98 (s, 1H), 8.15 [8.15] (s, 1H), 8.07-7.98 [8.07-7.98] (m, 1H), 7.70-7.59 [7.70-7.59] (m, 1H), 7.57-7.49 [7.57-7.49] (m, 1H), 7.42 [7.42] (s, 1H), 7.34-7.12 [7.34-7.12] (m, 3H), 4.93 [4.93] (q, J=5.7 Hz, 1H), 4.19 [4.19] (q, J=4.3 Hz, 2H), 3.81-3.75 [3.81-3.75] (m, 2H). m/z (ES⁺), [M+H]⁺ 398.4. HPLC (A05) t_{R}=2.02 min.

### Preparation of Compound A-53.

### Step 1: N-[5-(1H-benzimidazol-2-yl)-1-[(4-methoxyphenyl)methyl]pyrazol-3-yl]-3-chloro-4-(3-methoxypropoxy)benzamide

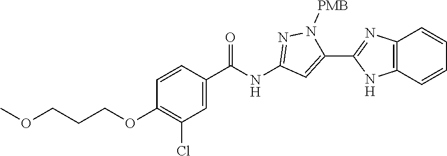

3-Chloro-4-(3-methoxypropoxy)benzoic acid (K-7) (80 mg, 0.327 mmol) and HATU (0.124 g, 0.327 mmol) were dissolved in DMF (3 mL) at 0° C. DIEA (0.112 mL, 0.654 mmol) was added, and the mixture was stirred at 0° C. for 5 min. 5-(1H-benzimidazol-2-yl)-1-[(4-methoxyphenyl)methyl]pyrazol-3-amine (J-1) (104 mg, 0.327 mmol) was added. The mixture was warmed to 23° C., and stirred for 18 h. EtOAc (200 mL) was added, and the organic phase was washed with water (4×50 mL) and brine (50 mL), dried (MgSO₄), filtered, and concentrated to provide the title compound as a solid (176 mg, 99%). ¹H NMR (400 MHz, DMSO) δ 13.13 (s, 1H), 11.07 (s, 1H), 8.15 (d, J=2.2 Hz, 1H), 8.04 (dd, J=8.7, 2.2 Hz, 1H), 7.72 (d, J=7.4 Hz, 1H), 7.57-7.52 (m, 2H), 7.32-7.20 (m, 5H), 6.88-6.83 (m, 2H), 6.03 (s, 2H), 4.20 (t, J=6.3 Hz, 2H), 3.68 (s, 3H), 3.51 (t, J=6.3 Hz, 2H), 3.26 (s, 3H), 2.05-1.97 (m, 2H). m/z (ES⁺), [M+H]⁺ 547.6. HPLC (A05) t_{R}=2.69 min.

### Step 2: N-[5-(1H-benzimidazol-2-yl)-1H-pyrazol-3-yl]-3-chloro-4-(3-methoxy-propoxy)benzamide

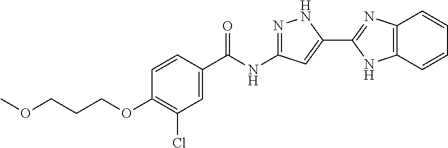 [SMILES: COCCCOc1ccc(C(=O)Nc2cc(-c3nc4ccccc4[nH]3)[nH]n2)cc1Cl]

A solution of N-[5-(1H-benzimidazol-2-yl)-1-[(4-methoxyphenyl)methyl]pyrazol-3-yl]-3-chloro-4-(3-methoxypropoxy)benzamide (176 mg, 0.322 mmol) in TFA (4 mL) was stirred at 23° C. for 1 h. The mixture was concentrated. The residue was dissolved in DMSO (0.500 mL). NEt₃ (0.500 mL) was added, and the mixture was stirred at 23° C. for 5 min. Water (25 mL) was added. The mixture was filtered, and the solid was purified by silica gel chromatography (12 g cartridge) eluting with DCM and MeOH (0-10%) to provide the title compound as a solid in a 3:1 mixture of pyrazole tautomers (69 mg, 50%). ¹H NMR (400 MHz, DMSO) δ 13.49 [13.16] (s, 1H), 13.03 [12.74] (s, 1H), [11.14] 10.99 (s, 1H), 8.18-8.11 [8.18-8.11] (m, 1H), 8.08-7.98 [8.08-7.98] (m, 1H), 7.71-7.60 [7.71-7.60] (m, 1H), 7.58-7.45 [7.58-7.45] (m, 1H), 7.45-7.36 [7.45-7.36] (m, 1H), 7.36-7.11 [7.36-7.11] (m, 3H), 4.21 [4.21] (t, J=6.2 Hz, 2H), 3.52 [3.52] (t, J=6.3 Hz, 2H), 3.26 [3.26] (s, 3H), 2.01 [2.01] (p, J=6.5 Hz, 2H). m/z (ES⁺), [M+H]⁺ 426.4. HPLC (A05) t_{R}=2.30 min.

### Preparation of Compound A-75.

### Step 1: N-[5-(1H-benzimidazol-2-yl)-1-[(4-methoxyphenyl)methyl]pyrazol-3-yl]-3-methoxy-4-(2-methoxyethoxy)benzamide

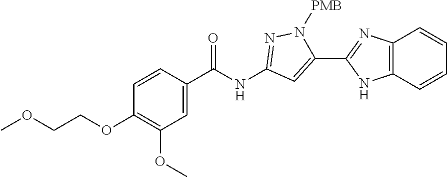

DIEA (191 μL, 1.10 mmol) was added to a solution of HATU (131 mg, 0.344 mmol) and 3-methoxy-4-(2-methoxyethoxy)benzoic acid (K-2) (74.4 mg, 0.329 mmol) in DMF (1.50 mL) at 0° C. The mixture was stirred at 0° C. for 15 min. 5-(1H-benzimidazol-2-yl)-1-[(4-methoxyphenyl)methyl]pyrazol-3-amine (J-1) (100 mg, 0.313 mmol) was added. The mixture was warmed to 23° C. and stirred for 18 h. Water (25 mL) was added, and the mixture was stirred at 23° C. for 5 min. The mixture was filtered, and the solid was purified by silica gel chromatography (25 g cartridge) eluting with hexanes and EtOAc (0-100%) to provide the title compound as a solid (96.5 mg, 58%). ¹H NMR (400 MHz, DMSO) δ 13.14 (s, 1H), 10.97 (s, 1H), 7.73 (d, J=7.5 Hz, 1H), 7.71-7.65 (m, 2H), 7.57 (s, 1H), 7.57-7.53 (m, 1H), 7.32-7.27 (m, 1H), 7.27-7.20 (m, 3H), 7.07 (d, J=8.5 Hz, 1H), 6.90-6.82 (m, 2H), 6.04 (s, 2H), 4.18-4.14 (m, 2H), 3.86 (s, 3H), 3.71-3.67 (m, 2H), 3.69 (s, 3H), 3.33 (s, 3H). m/z (ES⁺), [M+H]⁺ 528.6. HPLC (A05) t_{R}=2.48 min.

### Step 2: N-[5-(1H-benzimidazol-2-yl)-1H-pyrazol-3-yl]-3-methoxy-4-(2-methoxyethoxy)benzamide

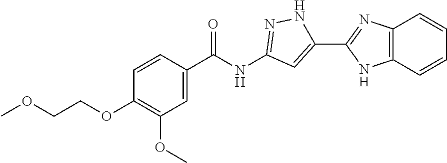 [SMILES: COCCOc1ccc(C(=O)Nc2cc(-c3nc4ccccc4[nH]3)[nH]n2)cc1OC]

TFA (1 mL) was added to N-[5-(1H-benzimidazol-2-yl)-1-[(4-methoxyphenyl)methyl]pyrazol-3-yl]-3-methoxy-4-(2-methoxyethoxy)benzamide (95 mg, 0.180 mmol), and the mixture was stirred at 23° C. for 30 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL) and dried under high vacuum to provide a 2:1 mixture of pyrazole tautomers of the title compound as a solid (39.8 mg, 54%). ¹H NMR (500 MHz, DMSO) δ 13.48 [13.16] (s, 1H), 13.03 [12.74] (s, 1H), 10.89 [11.02] (s, 1H), 7.76-7.59 [7.76-7.59] (m, 3H), 7.55 [6.67] (s, 1H), 7.43 [7.43] (s, 1H), 7.33-7.03 [7.33-7.03] (m, 3H), 4.33-3.98 [4.33-3.98] (m, 2H), 3.88 [3.88] (s, 3H), 3.78-3.63 [3.78-3.63] (m, 2H), 3.33 [3.33] (s, 3H). m/z (ES+), [M+H]⁺ 408.4. HPLC (A05) t_{R}=2.06 min.

### Preparation of Compound A-94.

### Step 1: N-[5-(1H-benzimidazol-2-yl)-1-[(4-methoxyphenyl)methyl]pyrazol-3-yl]-4-(2-methoxyethoxy)-3-methyl-benzamide

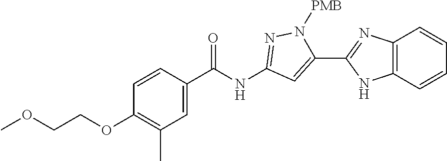

DIEA (143 μL, 0.822 mmol) was added to a solution of HATU (98.2 mg, 0.258 mmol) and 4-(2-methoxyethoxy)-3-methyl-benzoic acid (K-3) (51.8 mg, 0.247 mmol) in DMF (1 mL) at 0° C. The mixture was stirred at 0° C. for 10 min. 5-(1H-benzimidazol-2-yl)-1-[(4-methoxyphenyl)methyl]pyrazol-3-amine (J-1) (75 mg, 0.235 mmol) was added. The mixture was warmed to 23° C. and stirred for 18 h. Water (25 mL) was added, and the mixture was stirred at 23° C. for 5 min. The mixture was filtered, and the solid was purified by silica gel chromatography (25 g cartridge) eluting with hexanes and EtOAc (0-100%) to provide the title compound as a solid (74.7 mg, 62%). ¹H NMR (500 MHz, DMSO) δ 13.13 (s, 1H), 10.85 (s, 1H), 7.94-7.90 (m, 1H), 7.92 (s, 1H), 7.73 (d, J=8.0 Hz, 1H), 7.55 (s, 1H), 7.57-7.53 (m, 1H), 7.31-7.26 (m, 1H), 7.26-7.21 (m, 3H), 7.04 (d, J=9.0 Hz, 1H), 6.91-6.81 (m, 2H), 6.04 (s, 2H), 4.22-4.17 (m, 2H), 3.73-3.70 (m, 2H), 3.69 (s, 3H), 3.35 (s, 3H), 2.21 (s, 3H). m/z (ES⁺), [M+H]⁺ 512.4. HPLC (A05) t_{R}=2.60 min.

### Step 2: N-[5-(1H-benzimidazol-2-yl)-1H-pyrazol-3-yl]-4-(2-methoxyethoxy)-3-methyl-benzamide

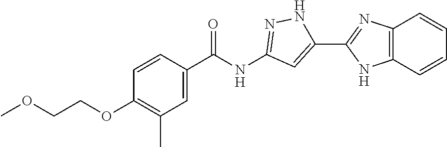 [SMILES: COCCOc1ccc(C(=O)Nc2cc(-c3nc4ccccc4[nH]3)[nH]n2)cc1C]

TFA (1 mL) was added to N-[5-(1H-benzimidazol-2-yl)-1-[(4-methoxyphenyl)methyl]pyrazol-3-yl]-4-(2-methoxyethoxy)-3-methyl-benzamide (72.5 mg, 0.142 mmol), and the mixture was stirred at 23° C. for 45 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL) and dried under high vacuum to provide a 5:3 mixture of pyrazole tautomers of the title compound as a solid (41.4 mg, 75%). ¹H NMR (500 MHz, DMSO) δ 13.45 [13.14] (s, 1H), 13.03 [12.74] (s, 1H), 10.77 [11.01] (s, 1H), 7.97-7.83 (m, 2H), 7.72-7.59 (m, 1H), 7.58-7.45 (m, 1H), 7.42 [6.68] (s, 1H), 7.31-7.13 (m, 2H), 7.12-7.00 (m, 1H), 4.42-3.92 (m, 2H), 3.90-3.57 (m, 2H), 3.35 (s, 3H), 2.24 (s, 3H). m/z (ES⁺), [M+H]⁺ 392.3. HPLC (A05) t_{R}=2.29 min.

### Preparation of Compound A-103.

### Step 1: N-[5-(1H-benzimidazol-2-yl)-1-[(4-methoxyphenyl)methyl]pyrazol-3-yl]-3-fluoro-4-(2-methoxyethoxy)benzamide

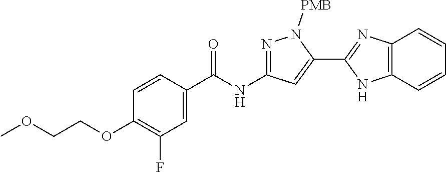

3-Fluoro-4-(2-methoxyethoxy)benzoic acid (K-4) (40.2 mg, 0.188 mmol) and HATU (0.0714 g, 0.188 mmol) were dissolved in DMF (1.50 mL) at 0° C. DIEA (0.0643 mL, 0.376 mmol) was added, and the mixture was stirred at 0° C. for 5 min. 5-(1H-benzimidazol-2-yl)-1-[(4-methoxyphenyl)methyl]pyrazol-3-amine (J-1) (60 mg, 0.188 mmol) was added, and the mixture was stirred at 23° C. for 18 h. EtOAc (100 mL) was added, and the organic phase was washed with water (3×50 mL) and brine (60 mL), dried (Na₂SO₄), filtered, and concentrated. The residue was purified by silica gel chromatography (12 g cartridge) eluting with hexanes and EtOAc (0-100%) to provide the title compound as a solid (81 mg, 84%). ¹H NMR (400 MHz, DMSO) δ 13.13 (s, 1H), 11.04 (s, 1H), 7.96-7.89 (m, 2H), 7.72 (d, J=7.9 Hz, 1H), 7.57-7.52 (m, 2H), 7.32-7.20 (m, 5H), 6.88-6.83 (m, 2H), 6.04 (s, 2H), 4.29-4.25 (m, 2H), 3.73-3.69 (m, 2H), 3.68 (s, 3H), 3.31 (s, 3H). m/z (ES⁺), [M+H]⁺ 516.1. HPLC (A05) t_{R}=2.53 min.

### Step 2: N-[5-(1H-benzimidazol-2-yl)-1H-pyrazol-3-yl]-3-fluoro-4-(2-methoxy-ethoxy)benzamide

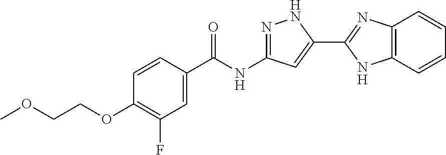 [SMILES: COCCOc1ccc(C(=O)Nc2cc(-c3nc4ccccc4[nH]3)[nH]n2)cc1F]

TFA (2 mL) was added to N-[5-(1H-benzimidazol-2-yl)-1-[(4-methoxyphenyl)methyl]pyrazol-3-yl]-3-fluoro-4-(2-methoxyethoxy)benzamide (81 mg, 0.157 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide the title compound as a solid in a 2:1 mixture of tautomers (39 mg, 63%). ¹H NMR (400 MHz, DMSO) δ 13.50 [13.17] (s, 1H), 13.03 [12.74] (s, 1H), [11.10] 10.95 (s, 1H), 7.91 (dd, J=17.9, 10.9 Hz, 2H), 7.67 [7.62] (d, J=7.7 Hz, 1H), 7.53 [7.46] (d, J=7.0 Hz, 1H), 7.42 [6.66] (s, 1H), 7.34-7.14 (m, 3H), 4.32-4.24 (m, 2H), 3.73-3.69 (m, 2H), 3.31 (s, 3H). ¹⁹F NMR (376 MHz, DMSO) δ −134.48 (s). m/z (ES⁺), [M+H]⁺ 396.2. HPLC (A05) t_{R}=2.13 min.

### Preparation of Compound A-104.

### Step 1: N-[5-(1H-benzimidazol-2-yl)-1-[(4-methoxyphenyl)methyl]pyrazol-3-yl]-4-(2-methoxyethoxy)-3-(trifluoromethyl)benzamide

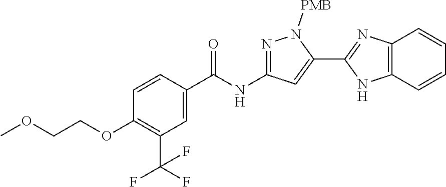

4-(2-Methoxyethoxy)-3-(trifluoromethyl)benzoic acid (K-5) (49.6 mg, 0.188 mmol) and HATU (0.0714 g, 0.188 mmol) were dissolved in DMF (1.50 mL) at 0° C. DIEA (0.0643 mL, 0.376 mmol) was added, and the mixture was stirred at 0° C. for 5 min. 5-(1H-benzimidazol-2-yl)-1-[(4-methoxyphenyl)methyl]pyrazol-3-amine (J-1) (60 mg, 0.188 mmol) was added, the mixture was stirred at 23° C. for 18 h. EtOAc (100 mL) was added, and the organic phase was washed with water (3×50 mL) and brine (60 mL), dried (MgSO₄), filtered, and concentrated. The residue was purified by silica gel chromatography (12 g cartridge) eluting with hexanes and EtOAc (0-100%) to provide the title compound as a solid (90 mg, 85%). ¹H NMR (400 MHz, DMSO) δ 13.14 (s, 1H), 11.24 (s, 1H), 8.37-8.30 (m, 2H), 7.72 (d, J=7.8 Hz, 1H), 7.57-7.52 (m, 2H), 7.40 (d, J=9.3 Hz, 1H), 7.31-7.21 (m, 4H), 6.88-6.83 (m, 2H), 6.04 (s, 2H), 4.37-4.31 (m, 2H), 3.72-3.69 (m, 2H), 3.69 (s, 3H), 3.33 (s, 3H). m/z (ES⁺), [M+H]⁺ 566.0. HPLC (A05) t_{R}=2.66 min.

### Step 2: N-[5-(1H-benzimidazol-2-yl)-1H-pyrazol-3-yl]-3-fluoro-4-(2-methoxy-ethoxy)benzamide

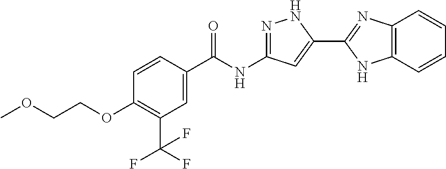 [SMILES: COCCOc1ccc(C(=O)Nc2cc(-c3nc4ccccc4[nH]3)[nH]n2)cc1C(F)(F)F]

TFA (2 mL) was added to N-[5-(1H-benzimidazol-2-yl)-1-[(4-methoxyphenyl)methyl]pyrazol-3-yl]-4-(2-methoxyethoxy)-3-(trifluoromethyl)benzamide (90 mg, 0.159 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide the title compound as a solid in a 3:1 mixture of tautomers (40.5 mg, 57%). ¹H NMR (400 MHz, DMSO) δ 13.52 [13.20] (s, 1H), 13.04 [12.75] (s, 1H), [11.25] 11.17 (s, 1H), 8.39-8.28 (m, 2H), 7.72-7.60 (m, 1H), 7.58-7.39 (m, 3H), 7.31-7.11 (m, 2H), 4.41-4.32 (m, 2H), 3.74-3.69 (m, 2H), 3.31 (s, 3H). ¹⁹F NMR (376 MHz, DMSO) δ −61.08 (s). m/z (ES⁺), [M+H]⁺ 446.2. HPLC (A05) t_{R}=2.29 min.

### Preparation of Compound A-113.

### Step 1: N-[5-(1H-benzimidazol-2-yl)-1-[(4-methoxyphenyl)methyl]pyrazol-3-yl]-4-(2-hydroxyethoxy)-3-methyl-benzamide

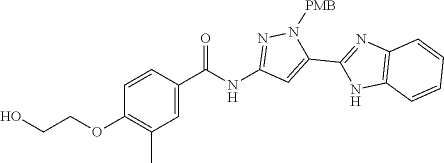

DIEA (143 μL, 0.822 mmol) was added to a solution of HATU (98.2 mg, 0.258 mmol) and 4-(2-hydroxyethoxy)-3-methyl-benzoic acid (K-10) (48.4 mg, 0.247 mmol) in DMF (1 mL) at 0° C. The mixture was stirred at 0° C. for 10 min. 5-(1H-benzimidazol-2-yl)-1-[(4-methoxyphenyl)methyl]pyrazol-3-amine (J-1) (75 mg, 0.235 mmol) was added. The mixture was warmed to 23° C. and stirred for 24 h. Water (25 mL) was added, and the mixture was stirred at 23° C. for 5 min. The mixture was filtered, and the solid was purified by silica gel chromatography (25 g cartridge) eluting with hexanes and EtOAc (0-100%) to provide the title compound as a solid (76 mg, 65%). ¹H NMR (500 MHz, DMSO) δ 13.14 (s, 1H), 10.85 (s, 1H), 7.95-7.89 (m, 2H), 7.73 (d, J=8.0 Hz, 1H), 7.57-7.53 (m, 2H), 7.32-7.27 (m, 1H), 7.26-7.22 (m, 3H), 7.02 (d, J=9.0 Hz, 1H), 6.89-6.84 (m, 2H), 6.04 (s, 2H), 4.88 (t, J=5.6 Hz, 1H), 4.11-4.07 (m, 2H), 3.79-3.74 (m, 2H), 3.69 (s, 3H), 2.23 (s, 3H). m/z (ES⁺), [M+H]⁺ 498.7. HPLC (A05) t_{R}=2.41 min.

### Step 2: N-[5-(1H-benzimidazol-2-yl)-1H-pyrazol-3-yl]-4-(2-hydroxyethoxy)-3-methyl-benzamide

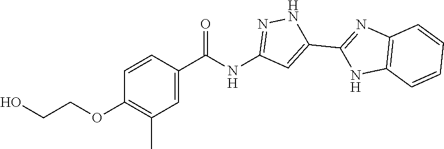 [SMILES: Cc1cc(C(=O)Nc2cc(-c3nc4ccccc4[nH]3)[nH]n2)ccc1OCCO]

TFA (1 mL) was added to N-[5-(1H-benzimidazol-2-yl)-1-[(4-methoxyphenyl)methyl]pyrazol-3-yl]-4-(2-hydroxyethoxy)-3-methyl-benzamide (74 mg, 0.149 mmol), and the mixture was stirred at 23° C. for 40 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with sat. NaHCO₃ (10 mL) and water (10 mL) and purified by silica gel chromatography (25 g cartridge) eluting with DCM and MeOH (0-15%) to provide a 2:1 mixture of pyrazole tautomers of the title compound as a solid (25.6 mg, 46%). ¹H NMR (500 MHz, DMSO) δ 13.37 [13.06] (s, 1H), 12.95 [12.66] (s, 1H), 10.17 [10.93] (s, 1H), 7.90-7.75 (m, 2H), 7.65-7.51 (m, 1H), 7.50-7.37 (m, 1H), 7.34 [6.58] (s, 1H), 7.23-7.06 (m, 2H), 7.06-6.92 (m, 1H), 4.91-4.68 (m, 1H), 4.08-3.96 (m, 2H), 3.76-3.61 (m, 2H), 2.17 (s, 3H). m/z (ES⁺), [M+H]⁺ 378.6. HPLC (A05) t_{R}=1.98 min.

### Preparation of Compound A-115.

### Step 1: 3-chloro-4-(2-hydroxyethoxy)-N-[1-[(4-methoxyphenyl)methyl]-5-(5-methyl-1H-benzimidazol-2-yl)pyrazol-3-yl]benzamide

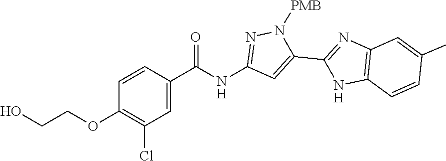

DIEA (137 μL, 0.787 mmol) was added to a solution of HATU (94.1 mg, 0.248 mmol) and 3-chloro-4-(2-hydroxyethoxy)benzoic acid (51.2 mg, 0.236 mmol) (K-8) in DMF (1 mL) at 0° C. The mixture was stirred at 0° C. for 10 min. 1-[(4-methoxyphenyl)methyl]-5-(5-methyl-1H-benzimidazol-2-yl)pyrazol-3-amine (J-6) (75 mg, 0.225 mmol) was added. The mixture was warmed to 23° C. and stirred for 18 h. Water (25 mL) was added, and the mixture was stirred at 23° C. for 5 min. The mixture was filtered, and the solid was purified by silica gel chromatography (16 g cartridge) eluting with DCM and MeOH (0-20%) to provide a 1:1 mixture of benzimidazole tautomers of the title compound as a solid (69.4 mg, 58%). ¹H NMR (500 MHz, DMSO) δ 12.99 [12.96] (s, 1H), 11.06 (s, 1H), 8.16 (d, J=2.2 Hz, 1H), 8.04 (dd, J=8.7, 2.2 Hz, 1H), 7.62-7.57 [7.53-7.51] (m, 1H), 7.52 (s, 1H), 7.45-7.40 [7.35-7.32] (m, 1H), 7.29 [7.27] (s, 1H), 7.26-7.22 (m, 2H), 7.13-7.09 [7.09-7.04] (m, 1H), 6.92-6.80 (m, 2H), 6.03 (s, 2H), 4.93 (t, J=5.4 Hz, 1H), 4.22-4.13 (m, 2H), 3.82-3.75 (m, 2H), 3.69 (s, 3H), 2.46 (s, 3H). m/z (ES⁺), [M+H]⁺ 532.7. HPLC (A05) t_{R}=2.50 min.

### Step 2: 3-chloro-4-(2-hydroxyethoxy)-N-[5-(5-methyl-1H-benzimidazol-2-yl)-1H-pyrazol-3-yl]benzamide

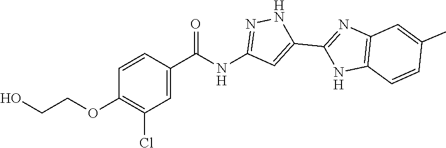 [SMILES: Cc1ccc2[nH]c(-c3cc(NC(=O)c4ccc(OCCO)c(Cl)c4)n[nH]3)nc2c1]

TFA (1 mL) was added to 3-chloro-4-(2-hydroxyethoxy)-N-[1-[(4-methoxyphenyl)methyl]-5-(5-methyl-1H-benzimidazol-2-yl)pyrazol-3-yl]benzamide (67.2 mg, 0.126 mmol), and the mixture was stirred at 23° C. for 40 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with sat. NaHCO₃ (10 mL) and water (10 mL) and purified by preparative HPLC (BEH C18 30×100) eluting with water ((10 mM (NH₄)(HCO₃)) (64-74%) to provide the title compound as a solid (11.3 mg, 22%). ¹H NMR (500 MHz, DMSO) δ 13.47 (s, 1H), 12.84 (s, 1H), 10.99 (s, 1H), 8.16 (d, J=2.0 Hz, 1H), 8.04 (d, J=6.6 Hz, 1H), 7.58-7.26 (m, 4H), 7.12-6.96 (m, 1H), 5.03-4.78 (m, 1H), 4.29-4.08 (m, J=4.9 Hz, 2H), 3.87-3.73 (m, 2H), 2.44 (s, 3H). m/z (ES⁺), [M+H]⁺ 412.6. HPLC (A05) t_{R}=2.11 min.

### Preparation of Compound A-116

### Step 1: 3-chloro-4-(2-hydroxyethoxy)-N-[1-[(4-methoxyphenyl)methyl]-5-[5-(trifluoromethoxy)-1H-benzimidazol-2-yl]pyrazol-3-yl]benzamide

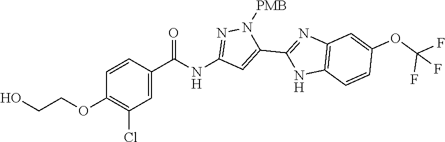

DIEA (137 μL, 0.787 mmol) was added to a solution of HATU (94.1 mg, 0.248 mmol) and 3-chloro-4-(2-hydroxyethoxy)benzoic acid (K-8) (51.2 mg, 0.236 mmol) in DMF (1 mL) at 0° C. The mixture was stirred at 0° C. for 10 min. 1-[(4-methoxyphenyl)methyl]-5-[5-(trifluoromethoxy)-1H-benzimidazol-2-yl]pyrazol-3-amine (J-9) (90.8 mg, 0.225 mmol) was added. The mixture was warmed to 23° C. and stirred for 18 h. Water (25 mL) was added, and the mixture was stirred at 23° C. for 5 min. The mixture was filtered, and the solid was purified by silica gel chromatography (16 g cartridge) eluting with DCM and MeOH (0-20%) to provide a 1:1 mixture of benzimidazole tautomers of the title compound as a solid (52.1 mg, 39%). ¹H NMR (500 MHz, DMSO) δ 13.45 (s, 1H), 11.10 (s, 1H), 8.16 (d, J=2.2 Hz, 1H), 8.04 (dd, J=8.7, 2.2 Hz, 1H), 7.86-7.80 [7.77-7.72] (m, 1H), 7.66-7.61 [7.54-7.49] (m, 1H), 7.59 (s, 1H), 7.29 [7.27] (s, 1H), 7.27-7.22 (m, 3H), 6.89-6.85 (m, 2H), 6.02 (s, 2H), 4.93 (br, 1H), 4.21-4.15 (m, 2H), 3.81-3.75 (m, 2H), 3.70 (s, 3H). ¹⁹F NMR (471 MHz, DMSO) δ −56.91 [−56.98]. m/z (ES⁺), [M+H]⁺ 602.7. HPLC (A05) t_{R}=2.63 min.

### Step 2: 3-chloro-4-(2-hydroxyethoxy)-N-[5-[5-(trifluoromethoxy)-1H-benzimidazol-2-yl]-1H-pyrazol-3-yl]benzamide

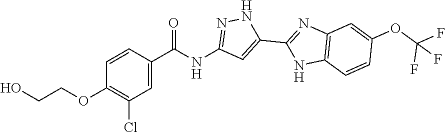 [SMILES: O=C(Nc1cc(-c2nc3cc(OC(F)(F)F)ccc3[nH]2)[nH]n1)c1ccc(OCCO)c(Cl)c1]

TFA (1 mL) was added to 3-chloro-4-(2-hydroxyethoxy)-N-[1-[(4-methoxyphenyl)methyl]-5-[5-(trifluoromethoxy)-1H-benzimidazol-2-yl]pyrazol-3-yl]benzamide (47.1 mg, 0.0782 mmol), and the mixture was stirred at 23° C. for 40 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with sat. NaHCO₃ (10 mL) and water (10 mL) and purified by preparative HPLC (BEH C18 30×100) eluting with water ((10 mM (NH₄)(HCO₃)) (70-80%) to provide a 2:1 mixture of pyrazole tautomers of the title compound as a solid (5.4 mg, 14%). ¹H NMR (500 MHz, DMSO) δ 13.58 (s, 1H), 13.32 (s, 1H), 11.03 (s, 1H), 8.15 (s, 1H), 8.04 (d, J=8.7 Hz, 1H), 7.79-7.69 (m, 1H), 7.69-7.57 (m, 1H), 7.51-7.42 (m, 1H), 7.38-7.27 (m, 1H), 7.27-7.18 (m, 1H), 5.00-4.88 (m, 1H), 4.25-4.15 (m, 2H), 3.85-3.75 (m, 2H). ¹⁹F NMR (471 MHz, DMSO) δ −56.96. m/z (ES⁺), [M+H]⁺ 482.5. HPLC (A05) t_{R}=2.27 min.

### Preparation of Compound A-117.

### Step 1: 3-chloro-4-(2-hydroxyethoxy)-N-[5-(5-methoxy-1H-benzimidazol-2-yl)-1-[(4-methoxyphenyl)methyl]pyrazol-3-yl]benzamide

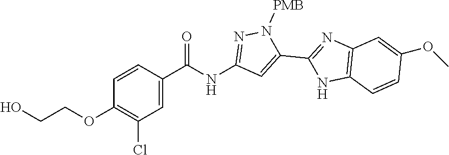

DIEA (137 μL, 0.787 mmol) was added to a solution of HATU (94.1 mg, 0.248 mmol) and 3-chloro-4-(2-hydroxyethoxy)benzoic acid (K-8) (51.2 mg, 0.236 mmol) in DMF (1 mL) at 0° C. The mixture was stirred at 0° C. for 10 min. 5-(5-methoxy-1H-benzimidazol-2-yl)-1-[(4-methoxyphenyl)methyl]pyrazol-3-amine (J-8) (78.6 mg, 0.225 mmol) was added. The mixture was warmed to 23° C. and stirred for 18 h. Water (25 mL) was added, and the mixture was stirred at 23° C. for 5 min. The mixture was filtered, and the solid was purified by silica gel chromatography (16 g cartridge) eluting with DCM and MeOH (0-20%) to provide a 1:1 mixture of benzimidazole tautomers of the title compound as a solid (74.3 mg, 60%). ¹H NMR (500 MHz, DMSO) δ 13.00 [12.97] (s, 1H), 11.05 (s, 1H), 8.16 (d, J=2.2 Hz, 1H), 8.04 (dd, J=8.7, 2.2 Hz, 1H), 7.62 [7.60] (s, 1H), 7.51 [7.50] (s, 1H), 7.43 [7.42] (s, 1H), 7.29 [7.27] (s, 1H), 7.26-7.22 (m, 2H), 7.00-6.98 [6.93-6.90] (m, 1H), 6.88-6.84 (m, 2H), 6.03 [6.01] (s, 2H), 4.93 (t, J=5.4 Hz, 1H), 4.21-4.15 (m, 2H), 3.84 [3.81] (s, 3H), 3.80-3.76 (m, 2H), 3.70 [3.70] (s, 3H). m/z (ES⁺), [M+H]⁺ 548.7. HPLC (A05) t_{R}=2.41 min.

### Step 2: 3-chloro-4-(2-hydroxyethoxy)-N-[5-(5-methoxy-1H-benzimidazol-2-yl)-1H-pyrazol-3-yl]benzamide formic acid

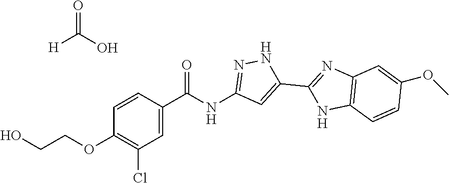 [SMILES: O=CO ; COc1ccc2[nH]c(-c3cc(NC(=O)c4ccc(OCCO)c(Cl)c4)n[nH]3)nc2c1]

TFA (1 mL) was added to 3-chloro-4-(2-hydroxyethoxy)-N-[5-(5-methoxy-1H-benzimidazol-2-yl)-1-[(4-methoxyphenyl)methyl]pyrazol-3-yl]benzamide (71.9 mg, 0.131 mmol), and the mixture was stirred at 23° C. for 40 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with sat. NaHCO₃ (10 mL) and water (10 mL) and purified by preparative HPLC (BEH C18 30×100) eluting with water ((10 mM (NH₄)(HCO₂)) (60-70%) to provide the title compound as a solid (11.3 mg, 18%). ¹H NMR (500 MHz, DMSO) δ 13.43 (s, 1H), 12.83 (s, 1H), 11.02 (s, 1H), 8.24 (s, 1H), 8.16 (d, J=2.1 Hz, 1H), 8.07-8.00 (m, 1H), 7.50 (s, 1H), 7.31 (d, J=8.9 Hz, 1H), 7.21-6.93 (m, 2H), 6.89-6.81 (m, 1H), 5.05-4.80 (m, 1H), 4.25-4.15 (m, 2H), 3.82 (s, 3H), 3.81-3.77 (m, 2H). m/z (ES⁺), [M+H]⁺ 428.5. HPLC (A05) t_{R}=2.03 min.

### Preparation of Compound A-118.

### Step 1: N-[5-(5-bromo-1H-benzimidazol-2-yl)-1-[(4-methoxyphenyl)methyl]-pyrazol-3-yl]-3-chloro-4-(2-hydroxyethoxy)benzamide

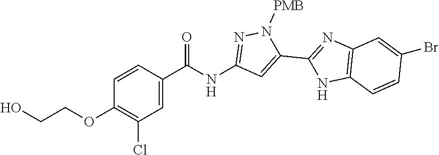

3-Chloro-4-(2-hydroxyethoxy)benzoic acid (K-8) (49.8 mg, 0.230 mmol) and HATU (0.0875 g, 0.230 mmol) were dissolved in DMF (1 mL) at 0° C. DIEA (0.0787 mL, 0.460 mmol) was added, and the mixture was stirred at 0° C. for 5 min. 5-(5-bromo-1H-benzimidazol-2-yl)-1-[(4-methoxyphenyl)methyl]pyrazol-3-amine (J-5) (91.6 mg, 0.230 mmol) was added, and the mixture was stirred at 23° C. for 18 h. EtOAc (100 mL) was added, and the organic phase was washed with water (3×50 mL) and brine (60 mL), dried (Na₂SO₄), filtered, and concentrated. The residue was purified by silica gel chromatography (12 g cartridge) eluting with DCM and MeOH (0-10%) to provide the title compound as a solid (93 mg, 68%). ¹H NMR (500 MHz, DMSO) δ 13.34 (s, 1H), 11.08 (s, 1H), 8.15 (d, J=2.3 Hz, 1H), 8.03 (dd, J=8.7, 2.3 Hz, 1H), 7.93 (s, 1H), 7.70 (s, 1H), 7.56 (s, 1H), 7.40 (s, 1H), 7.27 (d, J=8.8 Hz, 1H), 7.24 (d, J=8.8 Hz, 2H), 6.88-6.83 (m, 2H), 6.00 (s, 2H), 4.90 (s, 1H), 4.20-4.14 (m, 2H), 3.79-3.74 (m, 2H), 3.69 (s, 3H). m/z (ES⁺), [M+H]⁺ 598.1. HPLC (A05) t_{R}=2.60 min.

### Step 2: N-[5-(5-bromo-1H-benzimidazol-2-yl)-1H-pyrazol-3-yl]-3-chloro-4-(2-hydroxyethoxy)benzamide

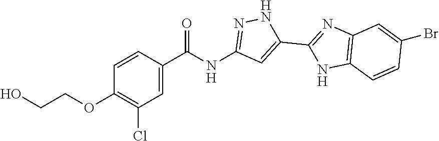 [SMILES: O=C(Nc1cc(-c2nc3cc(Br)ccc3[nH]2)[nH]n1)c1ccc(OCCO)c(Cl)c1]

TFA (1 mL) was added to N-[5-(5-bromo-1H-benzimidazol-2-yl)-1-[(4-methoxyphenyl)-methyl]pyrazol-3-yl]-3-chloro-4-(2-hydroxyethoxy)benzamide (93 mg, 0.156 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The dissolution-precipitation-wash process was repeated twice to provide the title compound as a solid (42.8 mg, 58%). ¹H NMR (400 MHz, DMSO) δ 13.54 (s, 1H), 13.22 (s, 1H), 11.04 (s, 1H), 8.14 (s, 1H), 8.02 (d, J=7.6 Hz, 1H), 7.82 (s, 1H), 7.49 (dd, J=40.2, 25.6 Hz, 2H), 7.39-7.27 (m, 2H), 4.94 (s, 1H), 4.19 (t, J=4.9 Hz, 2H), 3.78 (t, J=4.6 Hz, 2H). m/z (ES⁺), [M+H]⁺ 476.9. HPLC (A05) t_{R}=2.20 min.

### Preparation of Compound A-119.

### Step 1: 3-chloro-N-[5-(7-fluoro-1H-benzimidazol-2-yl)-1-[(4-methoxyphenyl)-methyl]pyrazol-3-yl]-4-(2-hydroxyethoxy)benzamide

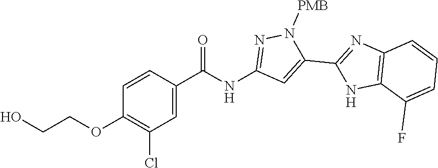

3-Chloro-4-(2-hydroxyethoxy)benzoic acid (K-8) (44.9 mg, 0.207 mmol) and HATU (0.0789 g, 0.207 mmol) were dissolved in DMF (1 mL) at 0° C. DIEA (0.0710 mL, 0.415 mmol) was added, and the mixture was stirred at 0° C. for 5 min. 5-(7-Fluoro-1H-benzimidazol-2-yl)-1-[(4-methoxyphenyl)methyl]pyrazol-3-amine (J-3) (70 mg, 0.207 mmol) was added, and the mixture was stirred at 23° C. for 18 h. EtOAc (100 mL) was added, and the organic phase was washed with water (3×50 mL) and brine (60 mL), dried (Na₂SO₄), filtered, and concentrated. The residue was purified by silica gel chromatography (12 g cartridge) eluting with hexanes and EtOAc (0-100%) to provide the title compound in a 7:2 mixture of tautomers as a solid (84.2 mg, 76%). ¹H NMR (400 MHz, DMSO) δ [13.64] 13.44 (s, 1H), 11.10 [11.07] (s, 1H), 8.16 (d, J=2.3 Hz, 1H), 8.04 (dd, J=8.7, 2.2 Hz, 1H), 7.58 (s, 1H), 7.40-7.35 (m, 1H), 7.29-7.19 (m, 4H), 7.06 (dd, J=11.4, 8.3 Hz, 1H), 6.89-6.83 (m, 2H), 6.01 (s, 2H), 4.93 (t, J=5.3 Hz, 1H), 4.18 (t, J=5.0 Hz, 2H), 3.77 (dd, J=10.1, 5.3 Hz, 2H), 3.69 (s, 3H). m/z (ES⁺), [M+H]+ 536.3. HPLC (A05) t_{R}=2.48 min.

### Step 2: 3-chloro-N-[5-(7-fluoro-1H-benzimidazol-2-yl)-1H-pyrazol-3-yl]-4-(2-hydroxyethoxy)benzamide

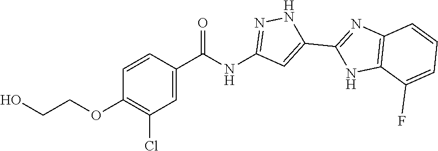 [SMILES: O=C(Nc1cc(-c2nc3cccc(F)c3[nH]2)[nH]n1)c1ccc(OCCO)c(Cl)c1]

TFA (2 mL) was added to 3-chloro-N-[5-(7-fluoro-1H-benzimidazol-2-yl)-1-[(4-methoxyphenyl)-methyl]pyrazol-3-yl]-4-(2-hydroxyethoxy)benzamide (84.2 mg, 0.157 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The residue was purified by preparative HPLC (BEH C18 30×150) eluting with water (10 mM (NH₄)(HCO₃)) and MeCN to provide the title compound as a solid (4.20 mg, 6%). ¹H NMR (400 MHz, DMSO) δ 13.47 (s, 1H), 11.06 (s, 1H), 8.15 (d, J=2.2 Hz, 1H), 8.04 (dd, J=8.6, 2.2 Hz, 1H), 7.32 (t, J=10.6 Hz, 2H), 7.17 (s, 1H), 6.98 (s, 1H), 4.93 (s, 1H), 4.19 (t, J=4.9 Hz, 2H), 3.78 (t, J=4.9 Hz, 2H). m/z (ES⁺), [M+H]⁺ 416.5. HPLC (A05) t_{R}=2.09 min.

### Preparation of Compound A-120.

### Step 1: N-[5-(1H-benzimidazol-2-yl)-1-[(4-methoxyphenyl)methyl]pyrazol-3-yl]-4-(2-hydroxyethoxy)-3-methoxy-benzamide

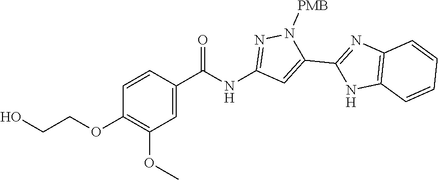

DIEA (143 μL, 0.822 mmol) was added to a mixture of HATU (98.2 mg, 0.258 mmol) and 4-(2-hydroxyethoxy)-3-methoxy-benzoic acid (K-9) (52.3 mg, 0.247 mmol) in DMF (1 mL) at 0° C. The mixture was stirred at 0° C. for 10 min and 5-(1H-benzimidazol-2-yl)-1-[(4-methoxyphenyl)methyl]pyrazol-3-amine (J-1) (75 mg, 0.235 mmol) was added. The mixture was stirred at 23° C. for 24 h and diluted with water (25 mL). The mixture was stirred at 23° C. for 5 min. The mixture was filtered, and the solid was washed with water (5 mL) and purified by silica gel chromatography (25 g cartridge) eluting first with hexanes and EtOAc (0-100%) and with DCM and MeOH (0-10%) to provide the title compound as a solid (50 mg, 41%). ¹H NMR (400 MHz, DMSO) δ 13.14 (s, 1H), 10.96 (s, 1H), 7.65 (dd, J=29.9, 21.5 Hz, 5H), 7.24 (d, J=8.6 Hz, 4H), 7.07 (d, J=8.4 Hz, 1H), 6.86 (d, J=8.7 Hz, 2H), 6.04 (s, 2H), 4.89 (t, J=5.4 Hz, 1H), 4.05 (t, J=5.0 Hz, 2H), 3.86 (s, 3H), 3.74 (dd, J=10.1, 5.1 Hz, 2H), 3.69 (s, 3H). m/z (ES⁻), [M−H]⁻ 512.6. HPLC (A05) t_{R}=2.32 min.

### Step 2: N-[5-(1H-benzimidazol-2-yl)-1H-pyrazol-3-yl]-4-(2-hydroxyethoxy)-3-methoxy-benzamide

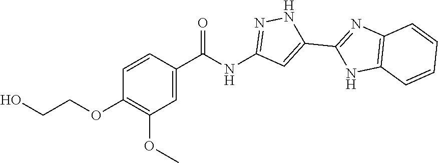 [SMILES: COc1cc(C(=O)Nc2cc(-c3nc4ccccc4[nH]3)[nH]n2)ccc1OCCO]

A mixture of N-[5-(1H-benzimidazol-2-yl)-1-[(4-methoxyphenyl)methyl]pyrazol-3-yl]-4-(2-hydroxyethoxy)-3-methoxy-benzamide (50 mg, 0.0974 mmol) in TFA (1 mL) was stirred at 23° C. for 30 min and concentrated. The residue was diluted in DMSO (0.500 mL) and NEt₃ (0.500 mL), and the mixture was stirred at 23° C. for 5 min. The mixture was diluted with water (10 mL). The mixture was filtered, and the solid was washed with acetone (5 mL), DCM (5 mL), and acetone (5 mL) to provide the title compound as a solid (9.00 mg, 23%). ¹H NMR (500 MHz, DMSO) δ 13.46 (s, 2H), 10.96 (s, 1H), 7.61 (s, 4H), 7.26 (s, 2H), 7.06 (s, 1H), 4.75 (s, 1H), 4.01 (s, 2H), 3.81 (s, 3H), 3.69 (s, 2H). m/z (ES⁺), [M+H]⁺ 394.2. HPLC (B30) t_{R}=1.04 min.

### Preparation of Compound A-121.

### Step 1: 3-chloro-N-[5-(5-chloro-1H-benzimidazol-2-yl)-1-[(4-methoxyphenyl)-methyl]pyrazol-3-yl]-4-(2-hydroxyethoxy)benzamide

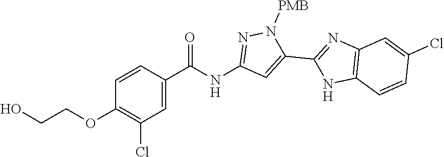

3-Chloro-4-(2-hydroxyethoxy)benzoic acid (K-8) (49.8 mg, 0.230 mmol) and HATU (0.0875 g, 0.230 mmol) were dissolved in DMF (1 mL) at 0° C. DIEA (0.0787 mL, 0.460 mmol) was added, and the mixture was stirred at 0° C. for 5 min. 5-(5-chloro-1H-benzimidazol-2-yl)-1-[(4-methoxyphenyl)methyl]pyrazol-3-amine (J-4) (81.4 mg, 0.230 mmol) was added, and the mixture was stirred at 23° C. for 18 h. EtOAc (100 mL) was added, and the organic phase was washed with water (3×50 mL) and brine (60 mL), dried (Na₂SO₄), filtered, and concentrated. The residue was purified by silica gel chromatography (12 g cartridge) eluting with DCM and MeOH (0-10%) to provide the title compound as a solid (94 mg, 74%). ¹H NMR (500 MHz, DMSO) δ 13.32 (s, 1H), 11.08 (s, 1H), 8.15 (d, J=2.3 Hz, 1H), 8.03 (dd, J=8.7, 2.2 Hz, 1H), 7.90-7.66 (m, 1H), 7.64-7.50 (m, 2H), 7.27 (d, J=8.8 Hz, 2H), 7.23 (d, J=8.8 Hz, 2H), 6.85 (d, J=8.8 Hz, 2H), 6.00 (s, 2H), 4.20-4.15 (m, 2H), 3.80-3.74 (m, 2H), 3.69 (s, 3H). m/z (ES⁺), [M+H]⁺ 553.2. HPLC (A05) t_{R}=2.57 min.

### Step 2: 3-chloro-N-[5-(5-chloro-1H-benzimidazol-2-yl)-1H-pyrazol-3-yl]-4-(2-hydroxyethoxy)benzamide

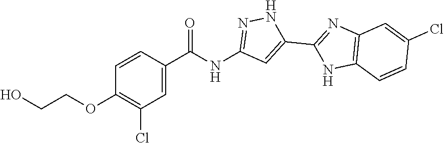 [SMILES: O=C(Nc1cc(-c2nc3cc(Cl)ccc3[nH]2)[nH]n1)c1ccc(OCCO)c(Cl)c1]

TFA (1 mL) was added to 3-chloro-N-[5-(5-chloro-1H-benzimidazol-2-yl)-1-[(4-methoxyphenyl)methyl]pyrazol-3-yl]-4-(2-hydroxyethoxy)benzamide (93 mg, 0.168 mmol), and the mixture was stirred at 23° C. for 40 min. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide 53 mg of the impure compound. 9 mg was withdrawn and purified by preparative HPLC (BEH C18 30×150) eluting with water (10 mM (NH₄)(HCO₂)) and MeCN (42-52%) to provide the title compound as a solid (4.40 mg, 48% from the mass withdrawn). ¹H NMR (400 MHz, DMSO) δ 8.36 (s, 1H), 8.15 (d, J=2.2 Hz, 1H), 8.04 (dd, J=8.7, 2.3 Hz, 1H), 7.61 (d, J=13.1 Hz, 1H), 7.31 (d, J=8.8 Hz, 1H), 7.23 (d, J=8.5 Hz, 1H), 4.94 (s, 1H), 4.19 (t, J=5.0 Hz, 2H), 3.78 (t, J=4.9 Hz, 2H). m/z (ES⁺), [M+H]⁺ 432.4. HPLC (A05) t_{R}=2.17 min.

### Preparation of Compound A-123.

### Step 1: 3-chloro-4-(2-hydroxyethoxy)-N-[1-[(4-methoxyphenyl)methyl]-5-[5-(trifluoromethyl)-1H-benzimidazol-2-yl]pyrazol-3-yl]benzamide

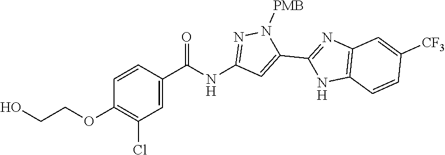

3-Chloro-4-(2-hydroxyethoxy)benzoic acid (K-8) (49.8 mg, 0.230 mmol) and HATU (0.0875 g, 0.230 mmol) were dissolved in DMF (1 mL) at 0° C. DIEA (0.0787 mL, 0.460 mmol) was added, and the mixture was stirred at 0° C. for 5 min. 1-[(4-methoxyphenyl)methyl]-5-[5-(trifluoromethyl)-1H-benzimidazol-2-yl]pyrazol-3-amine (J-7) (89.1 mg, 0.230 mmol) was added, and the mixture was stirred at 23° C. for 18 h. EtOAc (100 mL) was added, and the organic phase was washed with water (3×50 mL) and brine (60 mL), dried (Na₂SO₄), filtered, and concentrated. The residue was purified by silica gel chromatography (12 g cartridge) eluting with DCM and MeOH (0-10%) to provide the title compound as a solid (109 mg, 81%). ¹H NMR (500 MHz, DMSO) δ 13.58 (s, 1H), 11.11 (s, 1H), 8.16 (d, J=2.2 Hz, 1H), 8.10 [7.74] (s, 1H), 8.04 (dd, J=8.7, 2.3 Hz, 1H), 7.92-7.83 (m, 1H), 7.62 (s, 1H), 7.58 (s, 1H), 7.26 (t, J=8.9 Hz, 3H), 6.86 (d, J=8.8 Hz, 2H), 6.03 (s, 2H), 4.18 (t, J=5.0 Hz, 2H), 3.77 (t, J=5.0 Hz, 2H), 3.69 (s, 3H). m/z (ES⁺), [M+H]⁺ 587.1. HPLC (A05) t_{R}=2.61 min.

### Step 2: 3-chloro-4-(2-hydroxyethoxy)-N-[5-[5-(trifluoromethyl)-1H-benzimidazol-2-yl]-1H-pyrazol-3-yl]benzamide formic acid

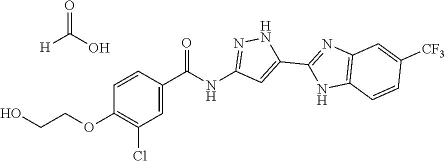 [SMILES: O=C(Nc1cc(-c2nc3cc(C(F)(F)F)ccc3[nH]2)[nH]n1)c1ccc(OCCO)c(Cl)c1 ; O=CO]

TFA (1 mL) was added to 3-chloro-4-(2-hydroxyethoxy)-N-[1-[(4-methoxyphenyl)methyl]-5-[5-(trifluoromethyl)-1H-benzimidazol-2-yl]pyrazol-3-yl]benzamide (109 mg, 0.186 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide 63.7 mg of the impure compound. 9 mg was withdrawn and purified by preparative HPLC (BEH C18 30×150) eluting with water (10 mM (NH₄)(HCO₂)) and MeCN (46-56%) to provide the title compound as a solid (5.40 mg, 58% from the mass withdrawn). ¹H NMR (400 MHz, DMSO) δ 8.37 (s, 1H), 8.15 (d, J=2.2 Hz, 1H), 8.04 (dd, J=8.7, 2.2 Hz, 1H), 7.91 (s, 1H), 7.75 (s, 1H), 7.53 (dd, J=8.3, 1.3 Hz, 1H), 7.32 (d, J=8.7 Hz, 1H), 4.95 (s, 1H), 4.19 (t, J=5.0 Hz, 2H), 3.78 (t, J=4.8 Hz, 2H). ¹⁹F NMR (376 MHz, DMSO) δ −58.86 (s). m/z (ES⁺), [M+H]⁺ 466.5. HPLC (A05) t_{R}=2.24 min.

### Preparation of Compound A-124

### Step 1: N-[5-(1H-benzimidazol-2-yl)-1-[(4-methoxyphenyl)methyl]pyrazol-3-yl]-3-fluoro-4-methoxy-benzamide

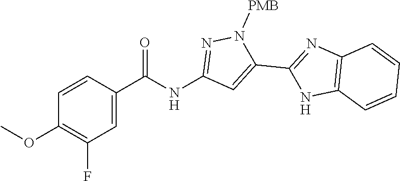

DIEA (143 μL, 0.822 mmol) was added to a solution of HATU (98.2 mg, 0.258 mmol) and 3-fluoro-4-methoxy-benzoic acid (42 mg, 0.247 mmol) in DMF (1 mL) at 0° C. The mixture was stirred at 0° C. for 10 min. 5-(1H-Benzimidazol-2-yl)-1-[(4-methoxyphenyl)methyl]pyrazol-3-amine (J-1) (75 mg, 0.235 mmol) was added. The mixture was warmed to 23° C. and stirred for 18 h. Water (25 mL) was added, and the mixture was stirred at 23° C. for 5 min. The mixture was filtered, and the solid was purified by silica gel chromatography (16 g cartridge) eluting with hexanes and EtOAc (0-100%) to provide the title compound as a solid (66.6 mg, 60%). ¹H NMR (500 MHz, DMSO) δ 13.14 (s, 1H), 11.05 (s, 1H), 7.97-7.89 (m, 2H), 7.73 (d, J=7.6 Hz, 1H), 7.56 (s, 1H), 7.56-7.54 (m, 1H), 7.33-7.21 (m, 5H), 6.89-6.82 (m, 2H), 6.04 (s, 2H), 3.93 (s, 3H), 3.70 (s, 3H). m/z (ES⁺), [M+H]⁺ 472.2. HPLC (A05) t_{R}=2.56 min.

### Step 2: N-[5-(1H-benzimidazol-2-yl)-1H-pyrazol-3-yl]-3-fluoro-4-methoxy-benzamide

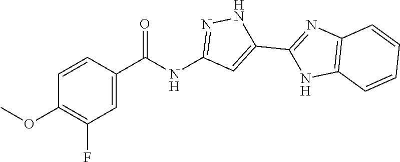 [SMILES: COc1ccc(C(=O)Nc2cc(-c3nc4ccccc4[nH]3)[nH]n2)cc1F]

TFA (1 mL) was added to 3-chloro-4-(2-hydroxyethoxy)-N-[5-(5-methoxy-1H-benzimidazol-2-yl)-1-[(4-methoxyphenyl)methyl]pyrazol-3-yl]benzamide (71.9 mg, 0.131 mmol), and the mixture was stirred at 23° C. for 40 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with sat. NaHCO₃ (10 mL) and water (10 mL) and purified by preparative HPLC (BEH C18 30×100) eluting with water ((10 mM (NH₄)(HCO₃)) (60-70%) to provide the title compound as a solid (11.3 mg, 18%). ¹H NMR (500 MHz, DMSO) δ 13.43 (s, 1H), 12.83 (s, 1H), 11.02 (s, 1H), 8.24 (s, 1H), 8.16 (d, J=2.1 Hz, 1H), 8.07-8.00 (m, 1H), 7.50 (s, 1H), 7.31 (d, J=8.9 Hz, 1H), 7.21-6.93 (m, 2H), 6.89-6.81 (m, 1H), 5.05-4.80 (m, 1H), 4.25-4.15 (m, 2H), 3.82 (s, 3H), 3.81-3.77 (m, 2H). m/z (ES⁺), [M+H]⁺ 428.5. HPLC (A05) t_{R}=2.03 min.

### Preparation of Compound A-129

### Step 1: N-[5-(1H-benzimidazol-2-yl)-1-[(4-methoxyphenyl)methyl]pyrazol-3-yl]-3-cyano-4-(2-methoxyethoxy)benzamide

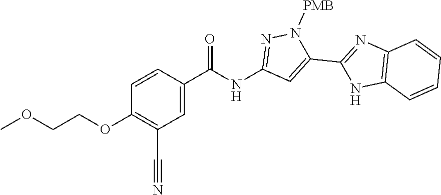

3-Cyano-4-(2-methoxyethoxy)benzoic acid (K-6) (61.1 mg, 0.276 mmol) and HATU (0.105 g, 0.276 mmol) were dissolved in DMF (1.50 mL) at 0° C. DIEA (0.0946 mL, 0.552 mmol) was added, and the mixture was stirred at 0° C. for 5 min. 5-(1H-Benzimidazol-2-yl)-1-[(4-methoxyphenyl)methyl]pyrazol-3-amine (J-1) (88.2 mg, 0.276 mmol) was added, and the mixture was stirred at 23° C. for 18 h. The mixture was concentrated and water (40 mL) was added to the residue. The aq. phase was extracted with EtOAc (3×40 mL). The combined organic phases were washed with brine (40 mL), dried (Na₂SO₄), filtered, and concentrated. The residue was purified by silica gel chromatography (12 g cartridge) eluting with hexanes and EtOAc (0-100%) to provide the title compound as a solid (93 mg, 64%). ¹H NMR (400 MHz, DMSO) δ 13.14 (s, 1H), 11.17 (s, 1H), 8.43 (d, J=2.3 Hz, 1H), 8.32 (dd, J=9.0, 2.3 Hz, 1H), 7.72 (d, J=7.5 Hz, 1H), 7.57-7.52 (m, 2H), 7.39 (d, J=9.1 Hz, 1H), 7.31-7.21 (m, 4H), 6.89-6.83 (m, 2H), 6.04 (s, 2H), 4.40-4.35 (m, 2H), 3.75-3.71 (m, 2H), 3.69 (s, 3H), 3.35 (s, 3H). m/z (ES⁺), [M+H]⁺ 523.2. HPLC (A05) t_{R}=2.52 min.

### Step 2: N-[5-(1H-benzimidazol-2-yl)-1H-pyrazol-3-yl]-3-cyano-4-(2-methoxyethoxy)benzamide

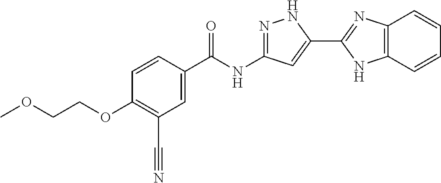 [SMILES: COCCOc1ccc(C(=O)Nc2cc(-c3nc4ccccc4[nH]3)[nH]n2)cc1C#N]

TFA (1 mL) was added to N-[5-(1H-benzimidazol-2-yl)-1-[(4-methoxyphenyl)methyl]pyrazol-3-yl]-3-cyano-4-(2-methoxyethoxy)benzamide (109 mg, 0.209 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide the title compound as a solid in a 4:1 mixture of tautomers (49.8 mg, 59%). ¹H NMR (400 MHz, DMSO) δ 13.53 [13.19] (s, 1H), 13.04 [12.75] (s, 1H), [11.21] 11.09 (s, 1H), 8.41 (d, J=19.1 Hz, 1H), 8.32 (d, J=9.1 Hz, 1H), 7.65 (t, J=14.8 Hz, 1H), 7.53 (d, J=7.2 Hz, 1H), 7.44 (dd, J=20.5, 15.0 Hz, 2H), 7.29-7.12 (m, 2H), 4.42-4.35 (m, 2H), 3.76-3.72 (m, 2H), 3.35 (s, 3H). m/z (ES⁺), [M+H]⁺ 403.2. HPLC (A05) t_{R}=2.12 min.

### Preparation of Compound A-144.

### Step 1: N-[5-(1H-benzimidazol-2-yl)-1-[(4-methoxyphenyl)methyl]pyrazol-3-yl]-3-cyano-4-(2-hydroxyethoxy)benzamide

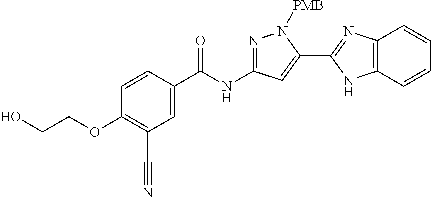

3-Cyano-4-(2-hydroxyethoxy)benzoic acid (K-13) (49.8 mg, 0.240 mmol) and HATU (0.0914 g, 0.240 mmol) were dissolved in DMF (1.50 mL) at 0° C. DIEA (0.0823 mL, 0.481 mmol) was added, and the mixture was stirred at 0° C. for 5 min. 5-(1H-Benzimidazol-2-yl)-1-[(4-methoxyphenyl)methyl]pyrazol-3-amine (J-1) (76.8 mg, 0.240 mmol) was added, the mixture was stirred at 23° C. for 18 h. EtOAc (100 mL) was added, and the organic phase was washed with water (3×50 mL) and brine (60 mL), dried (Na₂SO₄), filtered, and concentrated. The residue was purified by silica gel chromatography (12 g cartridge) eluting with DCM and MeOH (0-10%) to provide the title compound as a solid (56 mg, 46%). ¹H NMR (500 MHz, DMSO) δ 13.14 (s, 1H), 11.16 (s, 1H), 8.43 (d, J=2.2 Hz, 1H), 8.31 (dd, J=8.9, 2.4 Hz, 1H), 7.71 (s, 1H), 7.59-7.48 (m, 2H), 7.39 (d, J=9.1 Hz, 1H), 7.29-7.21 (m, 4H), 6.85 (d, J=8.8 Hz, 2H), 6.04 (s, 2H), 4.28-4.25 (m, 2H), 3.80-3.76 (m, 2H), 3.69 (s, 3H). m/z (ES⁺), [M+H]⁺ 509.2. HPLC (A05) t_{R}=2.37 min.

### Step 2: N-[5-(1H-benzimidazol-2-yl)-1H-pyrazol-3-yl]-3-cyano-4-(2-hydroxy-ethoxy)benzamide

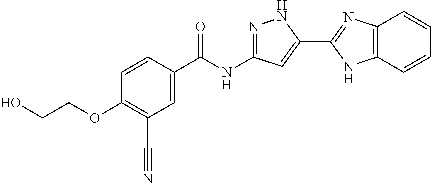 [SMILES: N#Cc1cc(C(=O)Nc2cc(-c3nc4ccccc4[nH]3)[nH]n2)ccc1OCCO]

TFA (1 mL) was added to N-[5-(1H-benzimidazol-2-yl)-1-[(4-methoxyphenyl)methyl]pyrazol-3-yl]-3-cyano-4-(2-hydroxyethoxy)benzamide (56 mg, 0.110 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The residue was purified by preparative HPLC (BEH C18 30×150) eluting with water (10 mM (NH₄)(HCO₃)) and MeCN (25-35%) to provide the title compound as a solid (11.7 mg, 27%). ¹H NMR (400 MHz, DMSO) δ 13.56 (s, 1H), 13.09 (s, 1H), 11.11 (s, 1H), 8.43 (d, J=2.3 Hz, 1H), 8.32 (dd, J=8.9, 2.3 Hz, 1H), 7.65 (s, 1H), 7.53 (s, 1H), 7.42 (d, J=9.0 Hz, 1H), 7.23 (s, 2H), 4.99 (s, 1H), 4.28 (t, J=4.8 Hz, 2H), 3.79 (t, J=4.7 Hz, 2H). m/z (ES⁺), [M+H]⁺ 389.6. HPLC (A05) t_{R}=1.96 min.

### Preparation of Compound A-152.

### Step 1: N-[5-(1H-benzimidazol-2-yl)-1-[(4-methoxyphenyl)methyl]pyrazol-3-yl]-4-(2-hydroxyethoxy)-3-(trifluoromethyl)benzamide

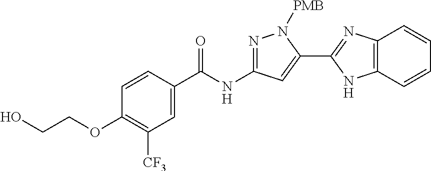

4-(2-Hydroxyethoxy)-3-(trifluoromethyl)benzoic acid (K-12) (78.3 mg, 0.313 mmol) and HATU (0.119 g, 0.313 mmol) were dissolved in DMF (2 mL) at 0° C. DIEA (0.107 mL, 0.626 mmol) was added, and the mixture was stirred at 0° C. for 5 min. 5-(1H-benzimidazol-2-yl)-1-[(4-methoxyphenyl)methyl]pyrazol-3-amine (J-1) (100 mg, 0.313 mmol) was added. The mixture was warmed to 23° C. and stirred for 18 h. The mixture was concentrated. EtOAc (150 mL) was added. The organic phase was washed with water (3×50 mL) and brine (50 mL), dried (Na₂SO₄), filtered, and concentrated. The residue was purified by silica gel chromatography (12 g cartridge) eluting with DCM and MeOH (0-10%) to provide the title compound as a solid (156 mg, 90%). ¹H NMR (400 MHz, DMSO) δ 13.14 (s, 1H), 11.23 (s, 1H), 8.37-8.30 (m, 2H), 7.72 (d, J=7.8 Hz, 1H), 7.57-7.52 (m, 2H), 7.40 (d, J=8.8 Hz, 1H), 7.31-7.19 (m, 4H), 6.88-6.82 (m, 2H), 6.04 (s, 2H), 4.89 (t, J=5.3 Hz, 1H), 4.23 (t, J=5.1 Hz, 2H), 3.76 (dd, J=10.2, 5.1 Hz, 2H), 3.68 (s, 3H). m/z (ES⁺), [M+H]⁺ 552.3. HPLC (A05) t_{R}=2.63 min.

### Step 2: N-[5-(1H-benzimidazol-2-yl)-1H-pyrazol-3-yl]-4-(2-hydroxyethoxy)-3-(trifluoromethyl)benzamide

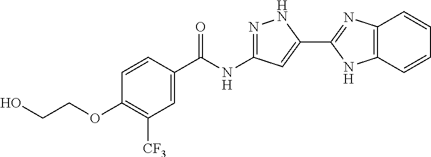 [SMILES: O=C(Nc1cc(-c2nc3ccccc3[nH]2)[nH]n1)c1ccc(OCCO)c(C(F)(F)F)c1]

TFA (2 mL) was added to N-[5-(1H-benzimidazol-2-yl)-1-[(4-methoxyphenyl)methyl]pyrazol-3-yl]-4-(2-hydroxyethoxy)-3-(trifluoromethyl)benzamide (155 mg, 0.281 mmol), and the mixture was stirred at 23° C. for 1 h. The mixture was concentrated. The residue was dissolved in DMSO (0.800 mL). NEt₃ (0.800 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The residue was purified by preparative HPLC (BEH C18 30×150) eluting with water (10 mM (NH₄)(HCO₃)) and MeCN (38-48%) to provide the title compound as a solid in a 3:1 mixture of tautomers (27.3 mg, 23%). ¹H NMR (400 MHz, DMSO) δ 13.49 [13.24] (s, 1H), 13.01 [12.73] (s, 1H), [11.27] 11.12 (s, 1H), 8.29 (d, J=11.9 Hz, 2H), 7.64 (d, J=7.0 Hz, 1H), 7.49 (d, J=7.2 Hz, 1H), 7.43-7.35 (m, 2H), 7.26-7.09 (m, 2H), 4.86 (t, J=5.3 Hz, 1H), 4.20 (t, J=4.7 Hz, 2H), 3.73 (dd, J=10.0, 5.0 Hz, 2H). ¹⁹F NMR (376 MHz, DMSO) δ −60.98 (s). m/z (ES⁺), [M+H]⁺ 432.6. HPLC (A05) t_{R}=2.10 min.

### EXAMPLE B13

### Preparation of Compounds A-21, A-26, A-41, A-42, A-44, A-45, A-46, A-47, A-48, A-49, A-51, A-52, A-54, A-55, A-56, A-59, A-60, A-61, A-63, A-64, A-65, A-66, A-67, A-68, A-69, A-73, A-80, A-81, A-82, A-83, A-84, A-85, A-86, A-87, A-88, A-89, A-90, A-91, A-98, A-99, A-100, A-101, A-102, A-105, A-107, A-108, A-109, A-110, A-111, A-114, A-131, A-132, A-142, A-143, A-156, A-159, A-169, A-170, A-171, A-172, A-177, A-179, A-180, A-183, A-186, A-188, A-192, A-193, A-194, A-218, and A-220

### Representative Synthetic Scheme for the Syntheses of the Title Compounds.

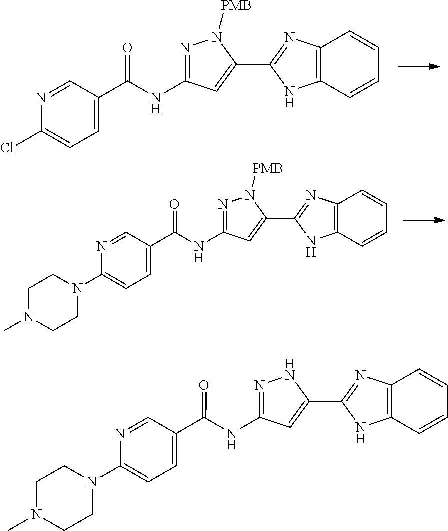

### Preparation of Compound A-21.

### Step 1: N-[5-(1H-benzimidazol-2-yl)-1-[(4-methoxyphenyl)methyl]-pyrazol-3-yl]-6-(4-methylpiperazin-1-yl)pyridine-3-carboxamide

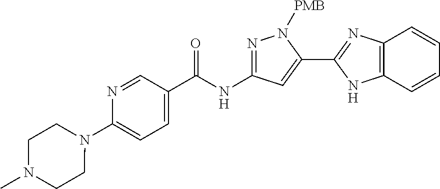

N-[5-(1H-Benzimidazol-2-yl)-1-[(4-methoxyphenyl)methyl]pyrazol-3-yl]-6-chloro-pyridine-3-carboxamide (140 mg, 0.305 mmol), freshly distilled 1-methylpiperazine (40.6 μL, 0.366 mmol) and DIEA (0.104 mL, 0.610 mmol) were dissolved in EtOH (2 mL). The vessel was capped, and the mixture was heated at 110° C. for 18 h. After cooling to 23° C., the mixture was concentrated. The residue was diluted in hexanes. The mixture was filtered, and the solid was dried to provide the title compound as a solid (116 mg, 73%). ¹H NMR (500 MHz, DMSO) δ 13.11 (s, 1H), 10.87 (s, 1H), 8.79 (d, J=2.4 Hz, 1H), 8.14 (dd, J=9.1, 2.6 Hz, 1H), 7.71 (d, J=8.2 Hz, 1H), 7.55-7.52 (m, 2H), 7.28 (t, J=7.5 Hz, 1H), 7.25-7.21 (m, 3H), 6.88 (d, J=9.0 Hz, 1H), 6.87-6.83 (m, 2H), 6.02 (s, 2H), 3.68 (s, 3H), 3.63 (s, 4H), 2.43 (s, 3H), 2.23 (d, J=13.9 Hz, 4H). m/z (ES⁺), [M+H]⁺ 523.2. HPLC (A05) t_{R}=2.07 min.

### Step 2: N-[5-(1H-benzimidazol-2-yl)-1H-pyrazol-3-yl]-6-(4-methyl-piperazin-1-yl)pyridine-3-carboxamide

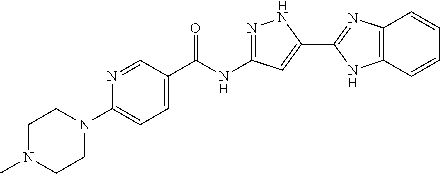 [SMILES: CN1CCN(c2ccc(C(=O)Nc3cc(-c4nc5ccccc5[nH]4)[nH]n3)cn2)CC1]

A solution of N-[5-(1H-benzimidazol-2-yl)-1-[(4-methoxyphenyl)methyl]pyrazol-3-yl]-6-(4-methylpiperazin-1-yl)pyridine-3-carboxamide (116 mg, 0.222 mmol) in TFA (3 mL) was heated to 70° C. for 30 min. The mixture was concentrated. Sat. NaHCO₃ (10 mL) and EtOAc (10 mL) were added, and the mixture was stirred at 23° C. for 10 min. The mixture was filtered, and the solid was purified by silica gel chromatography (25 g, silica cartridge) eluting with DCM and MeOH (0-20%)+2% NEt₃ to provide the title compound as a solid in a 3:1 ratio of the pyrazole tautomers (45 mg, 50%). ¹H NMR (500 MHz, DMSO) δ 13.45 [13.10] (s, 1H), 13.01 [12.73] (s, 1H), 10.79 [10.93] (s, 1H), 8.80 [8.80] (s, 1H), 8.16 [8.16] (d, J=8.8 Hz, 1H), 7.67 [7.67] (d, J=7.5 Hz, 1H), 7.53 [7.53] (d, J=7.6 Hz, 1H), 7.41 [7.41] (s, 1H), 7.28-7.19 [7.28-7.19] (m, 2H), 6.91 [6.91] (d, J=9.0 Hz, 1H), 3.74-3.54 [3.74-3.54] (m, 4H), 2.44 [2.44] (s, 3H), 2.36-2.15 [2.36-2.15] (m, 4H). m/z (ES⁺), [M+H]⁺ 403.4. HPLC (A05) t_{R}=1.72 min.

### Preparation of Compound A-26.

### Step 1: N-[5-(1H-benzimidazol-2-yl)-1-[(4-methoxyphenyl)methyl]pyrazol-3-yl]-5-chloro-6-(4-methylpiperazin-1-yl) pyridine-3-carboxamide

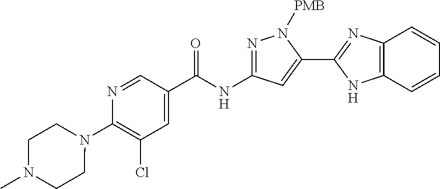

A mixture of N-[5-(1H-benzimidazol-2-yl)-1-[(4-methoxyphenyl)methyl] pyrazol-3-yl]-5,6-dichloro-pyridine-3-carboxamide (L-18) (139 mg, 0.282 mmol), freshly distilled 1-methylpiperazine (37.5 μL, 0.338 mmol), and DIEA (0.0965 mL, 0.564 mmol) in EtOH (4 mL) was heated at 110° C. in a sealed vessel for 16 h. The mixture was concentrated. Water (30 mL) was added, and the aq. phase was extracted with EtOAc (3×25 mL). The combined organic phases were washed with brine (15 mL), dried (MgSO₄), filtered, and concentrated. The residue was purified by silica gel chromatography (12 g, cartridge) eluting with DCM and MeOH (0-15%) to provide the title compound as a solid (137 mg, 87%). ¹H NMR (500 MHz, CDCl₃) δ 9.92 (s, 1H), 8.60 (d, J=2.2 Hz, 1H), 8.48 (s, 1H), 8.03 (d, J=2.2 Hz, 1H), 7.82 (d, J=6.8 Hz, 1H), 7.39 (s, 1H), 7.36 (d, J=7.9 Hz, 1H), 7.32-7.27 (m, 4H), 6.82-6.75 (m, 2H), 6.01 (s, 2H), 3.73 (s, 3H), 3.59 (t, J=5.0 Hz, 4H), 2.58 (t, J=5.0 Hz, 4H), 2.37 (s, 3H). m/z (ES⁺) [M+H]⁺: 557.0; HPLC (A05) t_{R}=2.11 min.

### Step 2: methyl 5-[(3-chloro-4-methoxy-phenyl)carbamoyl]-1-[(4-methoxyphenyl) methyl]pyrazole-3-carboxylate

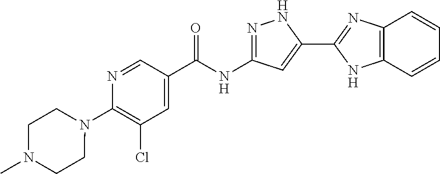 [SMILES: CN1CCN(c2ncc(C(=O)Nc3cc(-c4nc5ccccc5[nH]4)[nH]n3)cc2Cl)CC1]

A solution of N-[5-(1H-benzimidazol-2-yl)-1-[(4-methoxyphenyl)methyl]pyrazol-3-yl]-5-chloro-6-(4-methylpiperazin-1-yl) pyridine-3-carboxamide (135 mg, 0.242 mmol) in TFA (2 mL) was heated to 70° C. for 1 h. The mixture was concentrated. Sat. NaHCO₃ (10 mL) was added. The mixture was filtered, and the solid was washed with water (3×20 mL) and hexanes (2×25 mL). The solid was diluted in MeOH (10 mL). The mixture was filtered, and the solid was dried to provide the title compound as a solid (54.2 mg, 51%). ¹H NMR (500 MHz, DMSO) δ 13.47 (bs, 1H), 13.01 (bs, 1H), 11.09 (bs, 1H), 8.82 (d, J=1.9 Hz, 1H), 8.34 (d, J=1.8 Hz, 1H), 7.56 (bs, 2H), 7.38 (bs, 1H), 7.22 (s, 2H), 3.45 (t, J=5.0 Hz, 4H), 2.47 (t, J=5.0 Hz, 4H), 2.23 (s, 3H). m/z (ES⁺) [M+H]⁺:437.0; HPLC (A05) t_{R}=1.79 min.

### Preparation of Compound A-41.

### Step 1: N-[5-(1H-benzimidazol-2-yl)-1-[(4-methoxyphenyl)-methyl]pyrazol-3-yl]-6-morpholino-pyridine-3-carboxamide

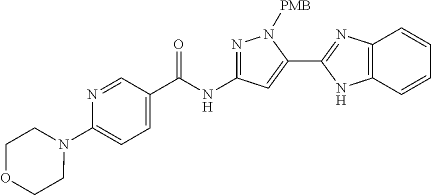

N-[5-(1H-Benzimidazol-2-yl)-1-[(4-methoxyphenyl)methyl]pyrazol-3-yl]-6-chloro-pyridine-3-carboxamide (L-1) (50 mg, 0.109 mmol), morpholine (14.2 mL, 0.163 mmol), and DIEA (37.3 μL, 0.218 mmol) were dissolved in DMSO (1 mL). The mixture stirred at 130° C. for 18 h. After cooling to 23° C., water (20 mL) was added. The aq. phase was extracted with EtOAc (2×40 mL). The combined organic phases were washed with water (3×20 mL) and brine (30 mL), dried (MgSO₄), filtered, and concentrated to provide the title compound as a solid (55 mg, 99%). ¹H NMR (400 MHz, DMSO) δ 13.12 (s, 1H), 10.90 (s, 1H), 8.81 (d, J=2.1 Hz, 1H), 8.17 (dd, J=9.0, 2.5 Hz, 1H), 7.72 (d, J=7.7 Hz, 1H), 7.54 (d, J=6.6 Hz, 2H), 7.30-7.20 (m, 4H), 6.88 (d, J=9.2 Hz, 1H), 6.87-6.83 (m, 2H), 6.03 (s, 2H), 3.72-3.67 (m, 7H), 3.61-3.57 (m, 4H). m/z (ES⁺), [M+H]⁺ 510.6. HPLC (A05) t_{R}=2.42 min.

### Step 2: N-[5-(1H-benzimidazol-2-yl)-1H-pyrazol-3-yl]-4-[(3S)-3-hydroxy-pyrrolidin-1-yl]benzamide

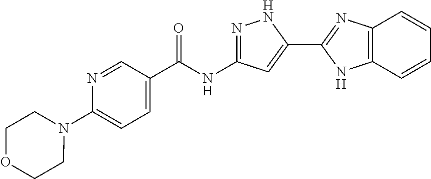 [SMILES: O=C(Nc1cc(-c2nc3ccccc3[nH]2)[nH]n1)c1ccc(N2CCOCC2)nc1]

A solution of N-[5-(1H-benzimidazol-2-yl)-1-[(4-methoxyphenyl)methyl]pyrazol-3-yl]-6-morpholino-pyridine-3-carboxamide (55 mg, 0.108 mmol) in TFA (3 mL) was stirred at 23° C. for 1 h. The mixture was concentrated. Sat. NaHCO₃ (10 mL) was added, and the aq. phase was extracted with EtOAc (3×20 mL). The combined organic phases were washed with brine (20 mL), dried (MgSO₄), filtered, and concentrated. The residue was purified by silica gel chromatography (12 g cartridge) eluting with DCM and MeOH (0-20%)+5% NEt₃, followed by reverse phase chromatography (BEH C18 30×150) eluting with water (10 mM (NH₄)(HCO₃)) and MeCN (31-41%) to provide the title compound as a solid (17.3 mg, 41%). ¹H NMR (400 MHz, DMSO) δ 13.47 (s, 1H), 13.18-12.72 (m, 1H), 10.83 (s, 1H), 8.82 (d, J=2.4 Hz, 1H), 8.33 (s, 1H), 8.17 (dd, J=9.0, 2.5 Hz, 1H), 7.64 (s, 1H), 7.51 (s, 1H), 7.22 (s, 2H), 6.92 (d, J=8.8 Hz, 1H), 3.73-3.68 (m, 4H), 3.61 (d, J=4.9 Hz, 4H). m/z (ES⁺), [M+H]⁺ 390.4. HPLC (A05) t_{R}=1.98 min.

### Preparation of Compound A-42.

### Step 1: N-[5-(1H-benzimidazol-2-yl)-1-[(4-methoxyphenyl)methyl]-pyrazol-3-yl]-6-(4-hydroxy-1-piperidyl)pyridine-3-carboxamide

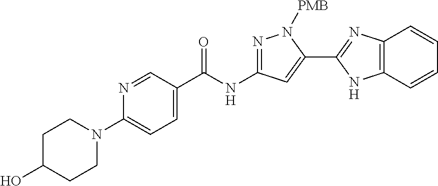

N-[5-(1H-Benzimidazol-2-yl)-1-[(4-methoxyphenyl)methyl]pyrazol-3-yl]-6-chloro-pyridine-3-carboxamide (L-1) (50 mg, 0.109 mmol), piperidin-4-ol (16.5 mg, 0.163 mmol), and DIEA (37.3 μL, 0.218 mmol) were dissolved in DMSO (1 mL). The mixture was stirred at 130° C. for 18 h. After cooling to 23° C., water (20 mL) was added. The aq. solution was extracted with EtOAc (2×40 mL). The combined organic phases were washed with water (3×20 mL) and brine (30 mL), dried (MgSO₄), filtered, and concentrated to provide the title compound as a solid (57 mg, 99%). ¹H NMR (400 MHz, DMSO) δ 13.11 (s, 1H), 10.83 (s, 1H), 8.77 (d, J=2.5 Hz, 1H), 8.11 (dd, J=9.1, 2.5 Hz, 1H), 7.71 (d, J=7.7 Hz, 1H), 7.56-7.51 (m, 2H), 7.30-7.20 (m, 4H), 6.89-6.82 (m, 3H), 6.02 (s, 2H), 4.72 (d, J=4.3 Hz, 1H), 4.10 (dt, J=9.1, 4.1 Hz, 2H), 3.74 (ddt, J=12.4, 8.1, 4.0 Hz, 1H), 3.68 (s, 3H), 3.23 (ddd, J=13.1, 9.8, 3.0 Hz, 2H), 1.82-1.74 (m, 2H), 1.40-1.30 (m, 2H). m/z (ES⁺), [M+H]⁺ 524.7. HPLC (A05) t_{R}=2.31 min.

### Step 2: N-[5-(1H-benzimidazol-2-yl)-1H-pyrazol-3-yl]-6-(4-hydroxy-1-piperidyl)pyridine-3-carboxamide

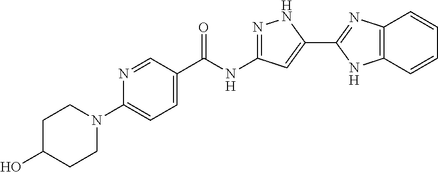 [SMILES: O=C(Nc1cc(-c2nc3ccccc3[nH]2)[nH]n1)c1ccc(N2CCC(O)CC2)nc1]

A solution of N-[5-(1H-benzimidazol-2-yl)-1-[(4-methoxyphenyl)methyl]pyrazol-3-yl]-6-(4-hydroxy-1-piperidyl)pyridine-3-carboxamide (57 mg, 0.109 mmol) in TFA (3 mL) was stirred at 23° C. for 1 h. The mixture was concentrated. Sat. NaHCO₃ (10 mL) was added, and the aq. phase was extracted with EtOAc (3×20 mL). The combined organic phases were washed with brine (20 mL), dried (MgSO₄), filtered, and concentrated. The residue was purified by silica gel chromatography (12 g cartridge) eluting with DCM and MeOH (0-30%) to provide the title compound as a solid (11.5 mg, 26%). ¹H NMR (400 MHz, DMSO) δ 13.39 (s, 1H), 12.98 (s, 1H), 10.71 (s, 1H), 8.73 (d, J=2.4 Hz, 1H), 8.07 (d, J=8.1 Hz, 1H), 7.66-7.44 (m, 2H), 7.36 (dd, J=18.9, 7.2 Hz, 1H), 7.24-7.09 (m, 2H), 6.86 (d, J=11.1 Hz, 1H), 4.69 (s, 1H), 4.07 (dt, J=12.8, 4.1 Hz, 2H), 3.76-3.66 (m, 1H), 3.19 (ddd, J=6.2, 4.0, 3.2 Hz, 2H), 1.79-1.71 (m, 2H), 1.37-1.26 (m, 2H). m/z (ES⁺), [M+H]⁺ 404.5. HPLC (A05) t_{R}=1.90 min.

### Preparation of Compound A-44.

### Step 1: N-[5-(1H-benzimidazol-2-yl)-1-[(4-methoxyphenyl)methyl]pyrazol-3-yl]-6-(1-oxa-8-azaspiro[4.5]decan-8-yl)pyridine-3-carboxamide

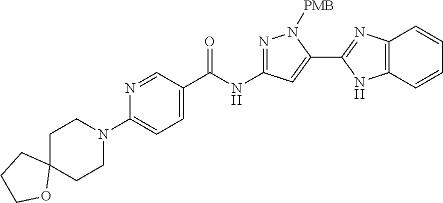

N-[5-(1H-benzimidazol-2-yl)-1-[(4-methoxyphenyl)methyl]pyrazol-3-yl]-6-chloro-pyridine-3-carboxamide (L-1) (50 mg, 0.109 mmol), 1-oxa-8-azaspiro[4.5]decane hydrochloride (29 mg, 0.163 mmol), and DIEA (74.6 μL, 0.436 mmol) were dissolved in DMSO (1 mL). The vessel was capped, and the mixture was heated at 130° C. for 18 h. After cooling to 23° C., water (20 mL) was added, and the aq. solution was extracted with EtOAc (2×40 mL). The combined organic phases were washed with water (3×20 mL) and brine (30 mL), dried (MgSO₄), filtered and concentrated. The residue was purified by silica gel chromatography (12 g cartridge) eluting with hexanes and EtOAc (40-100%) to provide the title compound as a solid (44 mg, 72%). ¹H NMR (400 MHz, DMSO) δ 13.11 (s, 1H), 10.84 (s, 1H), 8.77 (d, J=2.5 Hz, 1H), 8.12 (dd, J=9.1, 2.5 Hz, 1H), 7.71 (d, J=7.9 Hz, 1H), 7.53 (td, J=2.3, 0.9 Hz, 2H), 7.30-7.20 (m, 4H), 6.91-6.82 (m, 3H), 6.02 (s, 2H), 3.85 (dt, J=13.8, 4.7 Hz, 2H), 3.76 (t, J=6.7 Hz, 2H), 3.68 (s, 3H), 3.54 (ddd, J=13.0, 8.1, 4.6 Hz, 2H), 1.93-1.85 (m, 2H), 1.70 (dd, J=8.4, 6.5 Hz, 2H), 1.57 (dd, J=8.9, 4.2 Hz, 4H). m/z (ES⁺), [M+H]⁺ 565.6. HPLC (A05) t_{R}=2.59 min.

### Step 2: N-[5-(1H-benzimidazol-2-yl)-1H-pyrazol-3-yl]-6-(1-oxa-8-azaspiro[4.5]decan-8-yl)pyridine-3-carboxamide

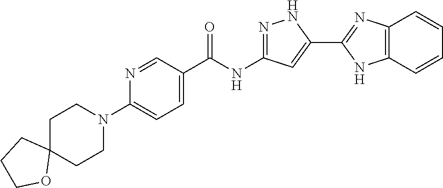 [SMILES: O=C(Nc1cc(-c2nc3ccccc3[nH]2)[nH]n1)c1ccc(N2CCC3(CCCO3)CC2)nc1]

A solution of N-[5-(1H-benzimidazol-2-yl)-1-[(4-methoxyphenyl)methyl]pyrazol-3-yl]-6-(1-oxa-8-azaspiro[4.5]decan-8-yl)pyridine-3-carboxamide (44 mg, 0.0781 mmol) in TFA (2 mL) was stirred at 23° C. for 1 h. The mixture was concentrated. Sat. NaHCO₃ (10 mL) was added. The mixture was stirred at 23° C. for 30 min. The mixture was filtered, and the solid was washed with water (10 mL) and dried under high vacuum to provide the title compound as a solid in a 7:3 ratio of tautomers (22.9 mg, 66%). ¹H NMR (500 MHz, DMSO) δ 13.43 [13.09] (s, 1H), 13.00 [12.72] (s, 1H), [10.89] 10.74 (s, 1H), 8.78 [8.76] (d, J=2.1 Hz, 1H), 8.13 [8.08] dd, J=9.1, 2.1 Hz, 1H), 7.67 [7.62] (d, J=7.5 Hz, 1H), 7.53 [7.46] (d, J=7.4 Hz, 1H), 7.40 [6.62] (s, 1H), 7.28-7.20 [7.20-7.13] (m, 2H), [6.97] 6.90 (d, J=9.6 Hz, 1H), 3.93-3.82 [3.93-3.82] (m, 2H), 3.77 [3.77] (t, J=6.8 Hz, 2H), 3.60-3.51 [3.60-3.51] (m, 2H), 1.94-1.86 [1.94-1.86] (m, 2H), 1.70 [1.70] (t, J=7.5 Hz, 2H), 1.62-1.53 [1.62-1.53] (m, 4H). m/z (ES⁺), [M+H]⁺ 444.9. HPLC (A05) t_{R}=2.17 min.

### Preparation of Compound A-45.

### Step 1: N-[5-(1H-benzimidazol-2-yl)-1-[(4-methoxyphenyl)methyl]pyrazol-3-yl]-6-(2-oxa-6-azaspiro[3.3]heptan-6-yl)pyridine-3-carboxamide

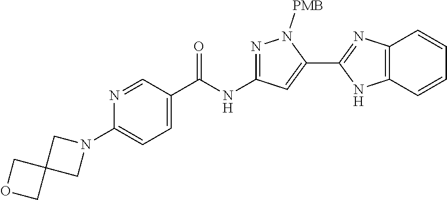

N-[5-(1H-Benzimidazol-2-yl)-1-[(4-methoxyphenyl)methyl]pyrazol-3-yl]-6-chloro-pyridine-3-carboxamide (50 mg, 0.109 mmol), 2-oxa-6-azaspiro[3.3]heptane (15.1 μL, 0.163 mmol), and DIEA (37.3 μL, 0.218 mmol) were dissolved in DMSO (1 mL). The mixture was heated at 130° C. for 18 h. After cooling to 23° C., water (20 mL) was added, and the aq. solution was extracted with EtOAc (2×40 mL). The combined organic phases were washed with water (3×20 mL) and brine (30 mL), dried (MgSO₄), filtered, and concentrated. The residue was purified by silica gel chromatography (12 g cartridge) eluting with hexanes and EtOAc (0-100%) to provide the title compound as a solid (28 mg, 49%). ¹H NMR (400 MHz, DMSO) δ 13.11 (s, 1H), 10.87 (s, 1H), 8.75 (dd, J=2.4, 0.7 Hz, 1H), 8.12 (dd, J=8.8, 2.4 Hz, 1H), 7.71 (d, J=7.6 Hz, 1H), 7.54 (d, J=6.6 Hz, 2H), 7.30-7.20 (m, 4H), 6.87-6.82 (m, 2H), 6.40 (d, J=8.8 Hz, 1H), 6.02 (s, 2H), 4.73 (s, 4H), 4.20 (s, 4H), 3.68 (s, 3H). m/z (ES⁺), [M+H]⁺ 522.6. HPLC (A05) t_{R}=2.31 min.

### Step 2: N-[5-(1H-benzimidazol-2-yl)-1H-pyrazol-3-yl]-6-(2-oxa-6-azaspiro[3.3]heptan-6-yl)pyridine-3-carboxamide

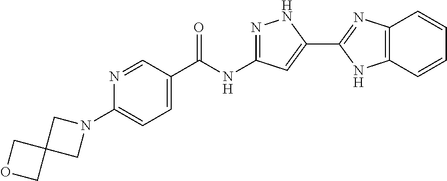 [SMILES: O=C(Nc1cc(-c2nc3ccccc3[nH]2)[nH]n1)c1ccc(N2CC3(COC3)C2)nc1]

A solution of N-[5-(1H-benzimidazol-2-yl)-1-[(4-methoxyphenyl)methyl]pyrazol-3-yl]-6-(2-oxa-6-azaspiro[3.3]heptan-6-yl)pyridine-3-carboxamide (28 mg, 0.0537 mmol) in TFA (2 mL) was stirred at 23° C. for 1 h. The mixture was concentrated. Sat. NaHCO₃ (10 mL) was added, and the mixture was stirred at 23° C. for 18 h. The mixture was filtered, and the solid was purified by reverse phase chromatography (BEH C18 30×150) eluting with water ((10 mM (NH₄)(HCO₃)) and MeCN (28-38%) to provide the title compound as a solid (6.40 mg, 30%). ¹H NMR (500 MHz, DMSO) δ 13.42 (s, 1H), 12.85 (s, 1H), 10.78 (s, 1H), 8.76 (d, J=2.2 Hz, 1H), 8.39 (s, 1H), 8.12 (dd, J=8.9, 2.0 Hz, 1H), 7.64 (s, 1H), 7.45 (d, J=60.5 Hz, 1H), 7.21 (s, 2H), 6.43 (d, J=7.6 Hz, 1H), 4.74 (s, 4H), 4.22 (s, 4H). m/z (ES⁺), [M+H]⁺ 402.4. HPLC (A05) t_{R}=1.90 min.

### Preparation of Compound A-46.

### Step 1: N-[5-(1H-benzimidazol-2-yl)-1-[(4-methoxyphenyl)methyl]pyrazol-3-yl]-6-[4-(dimethylamino)-1-piperidyl]pyridine-3-carboxamide

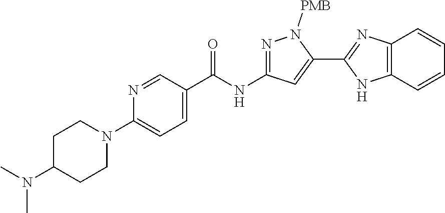

N-[5-(1H-Benzimidazol-2-yl)-1-[(4-methoxyphenyl)methyl]pyrazol-3-yl]-6-chloro-pyridine-3-carboxamide (L-1) (100 mg, 0.218 mmol), 4-dimethylaminopiperidine (51.3 μL, 0.436 mmol), and DIEA (149 μL, 0.872 mmol) were dissolved in DMSO (2 mL). The mixture was heated at 130° C. for 18 h. Water (50 mL) was added. The mixture was filtered, and the solid was purified by silica gel chromatography (16 g cartridge) eluting with mixtures of DCM and MeOH (0-10%)+1% NEt₃ to provide the title compound as a solid (103 mg, 86%). ¹H NMR (500 MHz, DMSO) δ 13.14 (s, 1H), 10.87 (s, 1H), 8.79 (d, J=2.5 Hz, 1H), 8.15 (dd, J=9.1, 2.5 Hz, 1H), 7.72 (d, J=7.9 Hz, 1H), 7.55 (s, 1H), 7.54 (d, J=5.8 Hz, 1H), 7.29 (t, J=7.6 Hz, 1H), 7.27-7.20 (m, 3H), 6.92 (d, J=9.2 Hz, 1H), 6.88-6.82 (m, 2H), 6.03 (s, 2H), 4.57-4.43 (m, 2H), 3.69 (s, 3H), 2.97-2.87 (m, 2H), 2.40 (br, 6H), 1.98-1.85 (m, 2H), 1.50-1.34 (m, 2H), 1.19-1.10 (m, 1H). m/z (ES⁺), [M+H]⁺ 551.7. HPLC (A05) t_{R}=2.09 min.

### Step 2: N-[5-(1H-benzimidazol-2-yl)-1H-pyrazol-3-yl]-6-[4-(dimethylamino)-1-piperidyl]pyridine-3-carboxamide

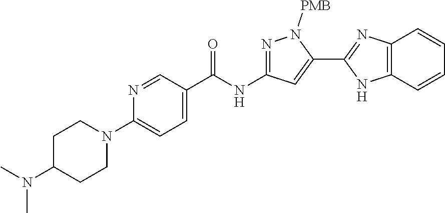

TFA (1 mL) was added to N-[5-(1H-benzimidazol-2-yl)-1-[(4-methoxyphenyl)methyl]-pyrazol-3-yl]-6-[4-(dimethylamino)-1-piperidyl]pyridine-3-carboxamide (100 mg, 0.182 mmol), and the mixture was stirred at 23° C. for 30 min. The mixture was concentrated. The residue was dissolved in DMSO (1 mL). NEt₃ (1 mL) was added, and the mixture was stirred at 23° C. for 5 min. Water (50 mL) was added. The solid was filtered and washed sat. NaHCO₃ (2×25 mL), water (2×25 mL), acetone (25 mL) and DCM (25 mL). The product was purified by Waters HPLC (BEH C-18 column, 30×150 mm, 5 micron) eluting with of water ((10 mM (NH₄)(HCO₃)) and MeCN (35-55%) to provide the title compound as a solid (19.7 mg, 25%). ¹H NMR (500 MHz, DMSO) δ 13.07 (br, 2H), 10.94 (br, 1H), 8.80 (d, J=2.4 Hz, 1H), 8.28 (s, 1H), 8.14 (dd, J=9.0, 2.5 Hz, 1H), 7.71-7.49 (m, 2H), 7.29-7.11 (m, 2H), 6.92 (d, J=9.3 Hz, 1H), 4.54-4.41 (m, 2H), 2.98-2.89 (m, 2H), 2.48-2.41 (m, 1H), 2.23 (s, 6H), 1.90-1.74 (m, 2H), 1.41-1.29 (m, 2H). m/z (ES⁺), [M+H]⁺ 431.6. HPLC (A05) t_{R}=1.75 min.

### Preparation of Compound A-47.

### Step 1: N-[5-(1H-benzimidazol-2-yl)-1-[(4-methoxyphenyl)methyl]pyrazol-3-yl]-6-[4-(1-hydroxy-1-methyl-ethyl)-1-piperidyl]pyridine-3-carboxamide

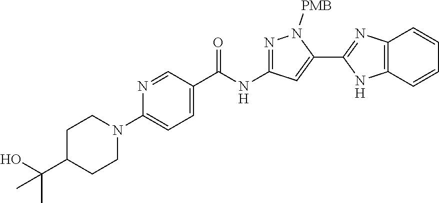

N-[5-(1H-Benzimidazol-2-yl)-1-[(4-methoxyphenyl)methyl]pyrazol-3-yl]-6-chloro-pyridine-3-carboxamide (L-1) (45 mg, 0.0981 mmol), 2-(4-piperidyl)propan-2-ol (21.1 mg, 0.147 mmol), and DIEA (33.6 μL, 0.196 mmol) were dissolved in DMSO (1 mL). The mixture was heated at 110° for 72 h. After cooling to 23° C., water (20 mL) was added, and the aq. phase was extracted with EtOAc (2×40 mL). The combined organic phases were washed with water (3×20 mL) and brine (30 mL), dried (MgSO₄), filtered, and concentrated to provide the title compound as a solid (45 mg, 81%). ¹H NMR (400 MHz, DMSO) δ 13.11 (s, 1H), 10.82 (s, 1H), 8.76 (d, J=2.6 Hz, 1H), 8.11 (dd, J=9.2, 2.4 Hz, 1H), 7.71 (d, J=7.4 Hz, 1H), 7.56-7.51 (m, 2H), 7.29-7.20 (m, 4H), 6.88-6.82 (m, 3H), 6.02 (s, 2H), 4.56-4.49 (m, 2H), 3.68 (s, 3H), 2.83-2.73 (m, 3H), 1.80-1.75 (m, 2H), 1.52-1.43 (m, 2H), 1.04 (s, 6H). m/z (ES⁺), [M+H]⁺ 566.8. HPLC (A05) t_{R}=2.45 min.

### Step 2: N-[5-(1H-benzimidazol-2-yl)-1H-pyrazol-3-yl]-6-[4-(1-hydroxy-1-methyl-ethyl)-1-piperidyl]pyridine-3-carboxamide

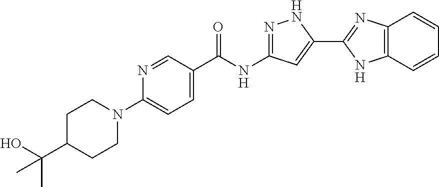 [SMILES: CC(C)(O)C1CCN(c2ccc(C(=O)Nc3cc(-c4nc5ccccc5[nH]4)[nH]n3)cn2)CC1]

A solution of N-[5-(1H-benzimidazol-2-yl)-1-[(4-methoxyphenyl)methyl]pyrazol-3-yl]-6-[4-(1-hydroxy-1-methyl-ethyl)-1-piperidyl]pyridine-3-carboxamide (45 mg, 0.0796 mmol) in TFA (2 mL) was stirred at 23° C. for 1 h. The mixture was concentrated. Sat. NaHCO₃ (10 mL) was added, and the mixture was stirred at 23° C. for 18 h. The mixture was filtered, and the solid was purified by reverse phase chromatography (BEH C18 30×150) eluting water (10 mM (NH₄)(HCO₃)) and MeCN (35-45%) to provide the title compound as a solid (17.1 mg, 48%). ¹H NMR (500 MHz, DMSO) δ 13.49 (s, 1H), 12.88 (s, 1H), 10.73 (s, 1H), 8.78 (d, J=2.4 Hz, 1H), 8.46 (s, 1H), 8.11 (dd, J=9.0, 2.5 Hz, 1H), 7.64 (s, 1H), 7.45 (d, J=57.0 Hz, 1H), 7.21 (s, 2H), 6.89 (d, J=6.3 Hz, 1H), 4.54 (d, J=12.2 Hz, 2H), 3.17 (s, 1H), 2.80 (t, J=12.3 Hz, 2H), 1.79 (d, J=11.7 Hz, 2H), 1.49 (tt, J=13.2, 3.6 Hz, 1H), 1.19 (ddd, J=25.7, 12.7, 4.1 Hz, 2H), 1.05 (s, 6H). m/z (ES⁺), [M+H]⁺ 446.7. HPLC (A05) t_{R}=2.05 min.

### Preparation of Compound A-48.

### Step 1: tert-butyl (25)-1-[5-[[5-(1H-benzimidazol-2-yl)-1-[(4-methoxyphenyl)-methyl]pyrazol-3-yl]carbamoyl]-2-pyridyl]pyrrolidine-2-carboxylate

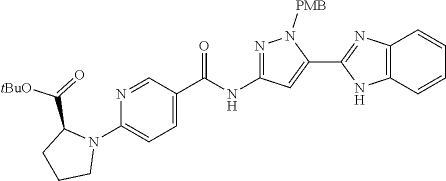

N-[5-(1H-Benzimidazol-2-yl)-1-[(4-methoxyphenyl)methyl]pyrazol-3-yl]-6-chloro-pyridine-3-carboxamide (L-1) (90 mg, 0.196 mmol), tert-butyl (2S)-pyrrolidine-2-carboxylate hydrochloride (61.1 mg, 0.294 mmol), and DIEA (0.134 mL, 0.784 mmol) were dissolved in DMSO (2 mL). The mixture was heated at 130° C. for 18 h. After cooling to 23° C., water (20 mL) was added, and the aq. phase was extracted with EtOAc (2×40 mL). The combined organic phases were washed with water (3×20 mL) and brine (30 mL), dried (MgSO₄), filtered, and concentrated. The residue was purified by silica gel chromatography (12 g cartridge) eluting with hexanes and EtOAc (0-80%) to provide the title compound as a solid (42 mg, 36%). ¹H NMR (400 MHz, DMSO) δ 13.11 (s, 1H), 10.86 (s, 1H), 8.73 (dd, J=2.2, 0.4 Hz, 1H), 8.16-8.13 (m, 1H), 7.71 (d, J=6.9 Hz, 1H), 7.56-7.52 (m, 2H), 7.32-7.19 (m, 5H), 6.87-6.83 (m, 2H), 6.02 (s, 2H), 4.44 (dd, J=9.5, 3.1 Hz, 1H), 3.68 (s, 3H), 3.52 (dd, J=10.2, 4.5 Hz, 2H), 2.04-1.93 (m, 4H), 1.38 (s, 9H). m/z (ES⁺), [M+H]⁺ 594.7. HPLC (A05) t_{R}=2.71 min.

### Step 2: (2S)-1-[5-[[5-(1H-benzimidazol-2-yl)-1H-pyrazol-3-yl]carbamoyl]-2-pyridyl]pyrrolidine-2-carboxylic acid

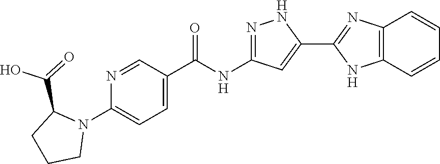 [SMILES: O=C(Nc1cc(-c2nc3ccccc3[nH]2)[nH]n1)c1ccc(N2CCC[C@H]2C(=O)O)nc1]

A solution of tert-butyl (2S)-1-[5-[[5-(1H-benzimidazol-2-yl)-1-[(4-methoxyphenyl)methyl]-pyrazol-3-yl]carbamoyl]-2-pyridyl]pyrrolidine-2-carboxylate (41.8 mg, 0.0704 mmol) in TFA (2 mL) was stirred at 23° C. for 1 h. The mixture was concentrated. Sat. NaHCO₃ (10 mL) was added, and the mixture was stirred at 23° C. for 30 min. The mixture was filtered, and the solid was washed with water (10 mL) and purified by reverse phase chromatography (BEH C18 30×150) eluting with water (10 mM (NH₄)(HCO₃)) and MeCN (18-38%) to provide the title compound as a solid (6.00 mg, 20%). ¹H NMR (400 MHz, DMSO) δ 13.42 (s, 1H), 13.02 (s, 1H), 12.64 (s, 1H), 10.68 (s, 1H), 8.69 (s, 1H), 8.19-8.00 (m, 1H), 7.71-7.22 (m, 3H), 7.22-7.06 (m, 2H), 6.66-6.37 (m, 1H), 4.61-4.16 (m, 1H), 3.52 (d, J=21.3 Hz, 2H), 2.24-2.13 (m, 1H), 2.05-1.92 (m, 3H). m/z (ES⁺), [M+H]⁺ 418.8. HPLC (A05) t_{R}=1.81 min.

### Preparation of Compound A-49.

### Step 1: N-[5-(1H-benzimidazol-2-yl)-1-[(4-methoxyphenyl)methyl]pyrazol-3-yl]-6-(1,1-dioxo-1,4-thiazinan-4-yl)pyridine-3-carboxamide

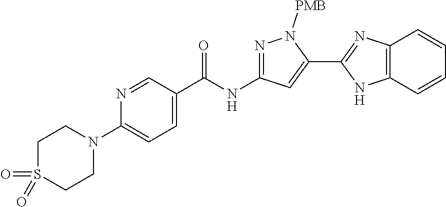

N-[5-(1H-Benzimidazol-2-yl)-1-[(4-methoxyphenyl)methyl]pyrazol-3-yl]-6-chloro-pyridine-3-carboxamide (L-1) (90 mg, 0.196 mmol), 1,4-thiazinane 1,1-dioxide hydrochloride (50.5 mg, 0.294 mmol), and DIEA (0.118 mL, 0.686 mmol) were dissolved in DMSO (2 mL). The mixture was heated at 130° C. for 18 h. After cooling to 23° C., water (20 mL) was added, and the aq. phase was extracted with EtOAc (2×40 mL). The combined organic phases were washed with water (3×20 mL) and brine (30 mL), dried (MgSO₄), filtered, and concentrated. The residue was purified by silica gel chromatography (12 g cartridge) eluting with hexanes and EtOAc (0-100%) to provide the title compound as a solid (48 mg, 44%). ¹H NMR (400 MHz, DMSO) δ 13.12 (s, 1H), 10.97 (s, 1H), 8.84 (d, J=3.1 Hz, 1H), 8.23 (dd, J=9.1, 2.4 Hz, 1H), 7.72 (d, J=7.6 Hz, 1H), 7.56-7.52 (m, 2H), 7.29-7.21 (m, 4H), 7.09 (d, J=9.4 Hz, 1H), 6.87-6.84 (m, 2H), 6.03 (s, 2H), 4.19-4.13 (m, 4H), 3.68 (s, 3H), 3.17-3.14 (m, 4H). m/z (ES⁺), [M+H]⁺ 558.6. HPLC (A05) t_{R}=2.36 min.

### Step 2: N-[5-(1H-benzimidazol-2-yl)-1H-pyrazol-3-yl]-6-(1,1-dioxo-1,4-thiazinan-4-yl)pyridine-3-carboxamide

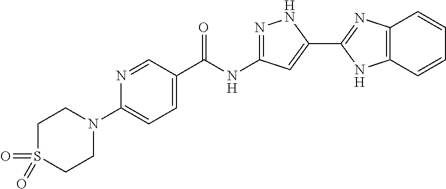 [SMILES: O=C(Nc1cc(-c2nc3ccccc3[nH]2)[nH]n1)c1ccc(N2CCS(=O)(=O)CC2)nc1]

A solution of N-[5-(1H-benzimidazol-2-yl)-1-[(4-methoxyphenyl)methyl]pyrazol-3-yl]-6-(1-oxa-8-azaspiro[4.5]decan-8-yl)pyridine-3-carboxamide (44 mg, 0.0781 mmol) in TFA (2 mL) was stirred at 23° C. for 1 h. The mixture was concentrated. Sat. NaHCO₃ (10 mL) was added, and the mixture was stirred at 23° C. for 30 min. The mixture was filtered, and the solid was washed with water (15 mL) and DCM (15 mL) and dried under high vacuum to provide the title compound as a solid (22 mg, 58%). ¹H NMR (500 MHz, DMSO) δ 13.46 [13.12] (s, 1H), 13.01 [12.73] (s, 1H), [11.01] 10.88 (s, 1H), 8.85 [8.82] (d, J=2.0 Hz, 1H), 8.24 [8.19] (dd, J=9.0, 2.0 Hz, 1H), 7.67 [7.62] (d, J=7.8 Hz, 1H), 7.53 [7.46] (d, J=7.8 Hz, 1H), 7.42 [7.42] (d, J=1.4 Hz, 1H), 7.27-7.15 [7.27-7.15] (m, 2H), 7.11 [7.11] (d, J=8.9 Hz, 1H), 4.20-4.13 [4.20-4.13] (m, 4H), 3.20-3.13 [3.20-3.13] (m, 4H). m/z (ES⁺), [M+H]⁺ 438.4. HPLC (A05) t_{R}=1.94 min.

### Preparation of Compound A-51.

### Step 1: 1-[5-[[5-(1H-benzimidazol-2-yl)-1-[(4-methoxyphenyl)methyl]-pyrazol-3-yl]carbamoyl]-2-pyridyl]piperidine-4-carboxylic acid

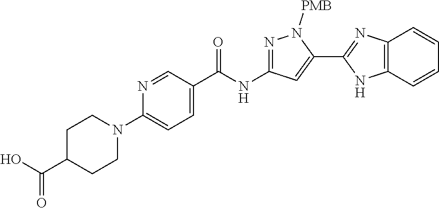

N-[5-(1H-Benzimidazol-2-yl)-1-[(4-methoxyphenyl)methyl]pyrazol-3-yl]-6-chloro-pyridine-3-carboxamide (L-1) (90 mg, 0.196 mmol), methyl piperidine-4-carboxylate (42.1 mg, 0.294 mmol), and DIEA (0.0671 mL, 0.392 mmol) were dissolved in DMSO (2 mL). The mixture was heated at 130° C. for 18 h. After cooling to 23° C., water (20 mL) was added, and the aq. phase was extracted with EtOAc (2×40 mL). The combined organic phases were washed with water (3×20 mL) and brine (30 mL), dried (MgSO₄), filtered, and concentrated. The residue was purified by silica gel chromatography (12 g cartridge) eluting with DCM and MeOH (0-40%) to provide the title compound as a solid (43 mg, 40%). ¹H NMR (400 MHz, DMSO) δ 13.07 (s, 1H), 12.20 (s, 1H), 10.81 (s, 1H), 8.73 (d, J=2.6 Hz, 1H), 8.08 (dd, J=9.1, 2.6 Hz, 1H), 7.67 (d, J=7.9 Hz, 1H), 7.51-7.47 (m, 2H), 7.26-7.15 (m, 4H), 6.86-6.77 (m, 3H), 5.98 (s, 2H), 4.27 (dt, J=6.3, 4.1 Hz, 2H), 3.64 (s, 3H), 3.24 (s, 1H), 3.02 (t, J=11.0 Hz, 2H), 1.88-1.80 (m, 2H), 1.47 (dd, J=20.6, 11.0 Hz, 2H). m/z (ES), [M−H]⁻ 550.6. HPLC (A05) t_{R}=2.35 min.

### Step 2: ammonium 1-[5-[[5-(1H-benzimidazol-2-yl)-1H-pyrazol-3-yl]carbamoyl]-2-pyridyl]piperidine-4-carboxylate

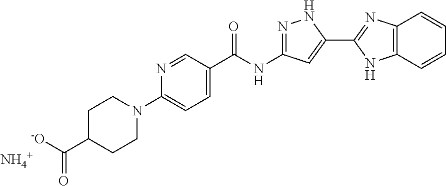

A solution of 1-[5-[[5-(1H-benzimidazol-2-yl)-1-[(4-methoxyphenyl)methyl]pyrazol-3-yl]carbamoyl]-2-pyridyl]piperidine-4-carboxylic acid (43 mg, 0.0780 mmol) in TFA (2 mL) was stirred at 23° C. for 1 h. The mixture was concentrated. The residue was dissolved in DMSO (0.500 mL) and NEt₃ (0.500 mL), and the mixture was stirred at 23° C. for 30 min. Water (10 mL) was added. The mixture was filtered, and the solid was purified by reverse phase chromatography (BEH C18 30×150) eluting with water (10 mM (NH₄)(HCO₃)) and MeCN (15-35%) to provide the title compound as a solid (14 mg, 40%). ¹H NMR (500 MHz, DMSO) δ 8.79 (s, 1H), 8.40 (s, 4H), 8.14 (d, J=9.4 Hz, 1H), 7.57 (s, 2H), 7.28-7.12 (m, 3H), 6.91 (d, J=8.8 Hz, 1H), 4.32 (d, J=12.9 Hz, 2H), 3.11-3.04 (m, 3H), 1.92-1.85 (m, 2H), 1.57-1.47 (m, 2H). m/z (ES⁺), [M+H−NH₃]⁺ 432.4. HPLC (A05) t_{R}=1.96 min.

### Preparation of Compound A-52.

### Step 1: N-[5-(1H-benzimidazol-2-yl)-1-[(4-methoxyphenyl)methyl]pyrazol-3-yl]-6-[(2S)-2-(hydroxymethyl)pyrrolidin-1-yl]pyridine-3-carboxamide

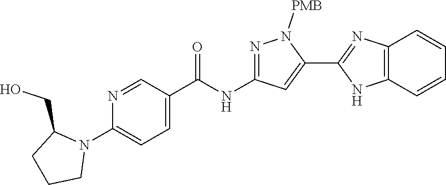

N-[5-(1H-Benzimidazol-2-yl)-1-[(4-methoxyphenyl)methyl]pyrazol-3-yl]-6-chloro-pyridine-3-carboxamide (L-1) (100 mg, 0.218 mmol), [(2S)-pyrrolidin-2-yl]methanol (32.3 μL, 0.327 mmol), and DIEA (74.6 mL, 0.436 mmol) were dissolved in DMSO (1 mL). The mixture was stirred at 100° C. for 18 h. Water (25 mL) was added. The mixture was filtered, and the solid was purified by silica gel chromatography eluting with DCM and MeOH (0-10%). The purified residue was diluted in hexanes (10 mL) and stirred for 5 min. The mixture was filtered, and the solid was dried under high vacuum to provide the title compound as a solid (66.8 mg, 59%). ¹H NMR (500 MHz, DMSO) δ 13.12 (s, 1H), 10.82 (s, 1H), 8.77 (d, J=2.2 Hz, 1H), 8.13 (dd, J=9.0, 2.4 Hz, 1H), 7.72 (d, J=7.7 Hz, 1H), 7.55 (s, 1H), 7.55 (d, J=6.5 Hz, 1H), 7.29 (t, J=7.2 Hz, 1H), 7.25-7.21 (m, 3H), 6.89-6.82 (m, 2H), 6.55 (d, J=9.2 Hz, 1H), 6.03 (s, 2H), 4.86 (t, J=5.7 Hz, 1H), 4.17-4.07 (m, 1H), 3.69 (s, 3H), 3.63-3.56 (m, 1H), 3.55-3.46 (m, 1H), 3.33-3.25 (m, 2H), 2.08-1.99 (m, 2H), 1.99-1.84 (m, 2H). m/z (ES⁺), [M+H]⁺ 524.7. HPLC (A05) t_{R}=2.37 min.

### Step 2: N-[5-(1H-benzimidazol-2-yl)-1H-pyrazol-3-yl]-6-[(2S)-2-(hydroxymethyl)-pyrrolidin-1-yl]pyridine-3-carboxamide

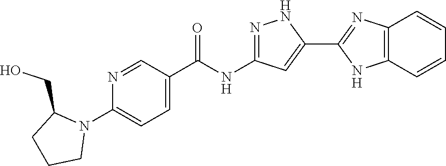 [SMILES: O=C(Nc1cc(-c2nc3ccccc3[nH]2)[nH]n1)c1ccc(N2CCC[C@H]2CO)nc1]

TFA (1 mL) was added to N-[5-(1H-benzimidazol-2-yl)-1-[(4-methoxyphenyl)methyl]-pyrazol-3-yl]-6-[(2S)-2-(hydroxymethyl)pyrrolidin-1-yl]pyridine-3-carboxamide (66.8 mg, 0.128 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 5 min. Water (25 mL) was added. The mixture was filtered, and the solid was washed with water (25 mL), acetone (25 mL), and DCM (25 mL) and dried under high vacuum to provide a 2:1 mixture of pyrazole tautomers of the title compound as a solid (36.8 mg, 72%). ¹H NMR (500 MHz, DMSO) δ 13.38 [13.00] (br, 1H), 13.00 [12.72] (br, 1H), 10.53 [10.76] (br, 1H), 8.02-7.86 [8.02-7.86] (m, 2H), 7.71-7.59 [7.71-7.59] (m, 1H), 7.58-7.44 [7.58-7.44] (m, 1H), 7.41 [6.60] (br, 1H), 7.30-7.11 [7.30-7.11] (m, 2H), 6.73-6.48 [6.73-6.48] (m, 2H), 5.02 [5.02] (br, 1H), 4.44 [4.44] (br, 1H), 3.54-3.46 [3.54-3.46] (m, 1H), 3.46-3.36 [3.46-3.36] (m, 2H), 3.25-3.13 [3.25-3.13] (m, 1H), 2.14-2.02 [2.14-2.02] (m, 1H), 1.99-1.90 [1.99-1.90] (m, 1H). m/z (ES⁺), [M+H]⁺ 404.4. HPLC (A05) t_{R}=1.96 min.

### Preparation of Compound A-54.

### Step 1: N-[5-(1H-benzimidazol-2-yl)-1-[(4-methoxyphenyl)methyl]pyrazol-3-yl]-6-[(3S)-3-hydroxypyrrolidin-1-yl]pyridine-3-carboxamide

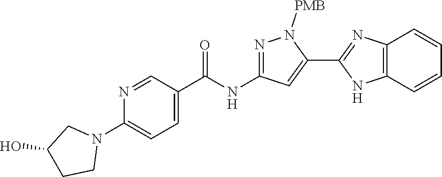

N-[5-(1H-Benzimidazol-2-yl)-1-[(4-methoxyphenyl)methyl]pyrazol-3-yl]-6-chloro-pyridine-3-carboxamide (L-1) (100 mg, 0.218 mmol), (3S)-pyrrolidin-3-ol (26.4 μL, 0.327 mmol), and DIEA (74.6 mL, 0.436 mmol) were dissolved in DMSO (1 mL). The mixture was stirred at 100° C. for 18 h. Water (25 mL) was added. The mixture was filtered, and the solid was purified by silica gel chromatography eluting with DCM and MeOH (0-10%). The purified residue was diluted in hexanes (25 mL) and stirred for 5 min. The mixture was filtered, and the solid was dried under high vacuum to provide the title compound as a solid (62.2 mg, 56%). ¹H NMR (500 MHz, DMSO) δ 13.12 (s, 1H), 10.80 (s, 1H), 8.78 (d, J=2.1 Hz, 1H), 8.12 (dd, J=9.0, 2.5 Hz, 1H), 7.72 (d, J=7.8 Hz, 1H), 7.55 (s, 1H), 7.54 (d, J=5.1 Hz, 1H), 7.28 (t, J=7.6 Hz, 1H), 7.26-7.20 (m, 3H), 6.88-6.83 (m, 2H), 6.48 (d, J=9.0 Hz, 1H), 6.03 (s, 2H), 5.00 (d, J=3.5 Hz, 1H), 4.44-4.38 (m, 1H), 3.69 (s, 3H), 3.59-3.47 (m, 3H), 3.44-3.37 (m, 1H), 2.10-1.99 (m, 1H), 1.97-1.86 (m, 1H). m/z (ES⁺), [M+H]⁺ 510.6. HPLC (A05) t_{R}=2.25 min.

### Step 2: N-[5-(1H-benzimidazol-2-yl)-1H-pyrazol-3-yl]-6-[(3S)-3-hydroxy-pyrrolidin-1-yl]pyridine-3-carboxamide

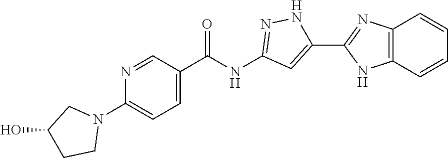

TFA (1 mL) was added to N-[5-(1H-benzimidazol-2-yl)-1-[(4-methoxyphenyl)methyl]pyrazol-3-yl]-6-[(3S)-3-hydroxypyrrolidin-1-yl]pyridine-3-carboxamide (60 mg, 0.118 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 5 min. Water (25 mL) was added. The mixture was filtered, and the solid was washed with water (25 mL), acetone (25 mL), DCM (25 mL), and acetone (25 mL) and dried under high vacuum to provide the title compound as a solid (32.1 mg, 70%) . ¹H NMR (500 MHz, DMSO) δ 13.45 (br, 1H), 13.02 (br, 1H), 10.73 (br, 1H), 8.79 (s, 1H), 8.34-7.90 (m, 1H), 7.80-7.34 (m, 3H), 7.34-7.10 (m, 2H), 6.64-6.38 (m, 1H), 5.23-4.78 (m, 1H), 4.55-4.23 (m, 1H), 3.65-3.47 (m, 4H), 2.15-1.97 (m, 1H), 1.97-1.77 (m, 1H). m/z (ES⁺), [M+H]⁺ 390.4. HPLC (A05) t_{R}=1.85 min.

### Preparation of Compound A-55.

### Step 1: N-[5-(1H-benzimidazol-2-yl)-1-[(4-methoxyphenyl)methyl]pyrazol-3-yl]-6-[(3R)-3-hydroxypyrrolidin-1-yl]pyridine-3-carboxamide

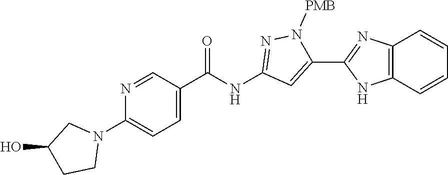

N-[5-(1H-Benzimidazol-2-yl)-1-[(4-methoxyphenyl)methyl]pyrazol-3-yl]-6-chloro-pyridine-3-carboxamide (L-1) (100 mg, 0.218 mmol), (3R)-pyrrolidin-3-ol (26.4 μL, 0.327 mmol), and DIEA (74.6 mL, 0.436 mmol) were dissolved in DMSO (1 mL). The mixture was heated at 100° C. for 18 h. Water (25 mL) was added. The mixture was filtered, and the solid was purified by silica gel chromatography eluting with DCM and MeOH (0-10%). The purified residue was diluted in hexanes (25 mL) and stirred for 5 min. The mixture was filtered, and the solid was dried under high vacuum to provide the title compound as a solid (33.8 mg, 30%). ¹H NMR (500 MHz, DMSO) δ 13.12 (s, 1H), 10.80 (s, 1H), 8.79 (d, J=2.1 Hz, 1H), 8.13 (dd, J=9.0, 2.5 Hz, 1H), 7.72 (d, J=8.2 Hz, 1H), 7.55 (s, 1H), 7.54 (d, J=5.2 Hz, 1H), 7.29 (t, J=7.8 Hz, 1H), 7.26-7.20 (m, 3H), 6.89-6.82 (m, 2H), 6.49 (d, J=8.9 Hz, 1H), 6.03 (s, 2H), 5.00 (d, J=3.4 Hz, 1H), 4.45-4.37 (m, 1H), 3.69 (s, 3H), 3.58-3.47 (m, 3H), 3.44-3.36 (m, 1H), 2.09-1.99 (m, 1H), 1.97-1.87 (m, 1H). m/z (ES⁺), [M+H]⁺ 510.6. HPLC (A05) t_{R}=2.25 min.

### Step 2: N-[5-(1H-benzimidazol-2-yl)-1H-pyrazol-3-yl]-6-[(3R)-3-hydroxypyrrolidin-1-yl]pyridine-3-carboxamide

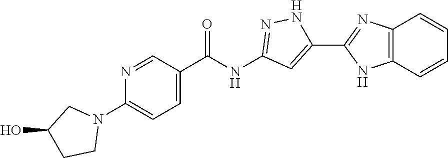

TFA (1 mL) was added to N-[5-(1H-benzimidazol-2-yl)-1-[(4-methoxyphenyl)methyl]pyrazol-3-yl]-6-[(3S)-3-hydroxypyrrolidin-1-yl]pyridine-3-carboxamide (30 mg, 0.0589 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 5 min. Water (25 mL) was added. The mixture was filtered, and the solid was washed with water (25 mL), acetone (25 mL), DCM (25 mL), and acetone (25 mL) and dried under high vacuum to provide a 2:1 mixture of pyrazole tautomers of the title compound as a solid (16.8 mg, 73%). ¹H NMR (500 MHz, DMSO) δ 13.43 [13.08] (s, 1H), 13.01 [12.73] (s, 1H), 10.71 [10.87] (s, 1H), 8.84-8.73 [8.84-8.73] (m, 1H), 8.16-8.11 [8.11-8.06] (m, 1H), 7.72-7.65 [7.65-7.59] (m, 1H), 7.58-7.51 [7.50-7.45] (m, 1H), 7.41 [6.63] (s, 1H), 7.31-7.21 [7.21-7.12] (m, 2H), 6.54-6.46 [6.61-6.54] (m, 1H), 5.05-4.98 [5.05-4.98] (m, 1H), 4.48-4.39 [4.48-4.39] (m, 1H), 3.63-3.46 [3.63-3.46] (m, 4H), 2.12-2.00 [2.12-2.00] (m, 1H), 2.00-1.86 [2.00-1.86] (m, 1H). m/z (ES⁺), [M+H]⁺ 390.4. HPLC (A05) t_{R}=1.85 min.

### Preparation of Compound A-56.

### Step 1: ethyl (2S)-1-[5-[[5-(1H-benzimidazol-2-yl)-1-[(4-methoxyphenyl)-methyl]pyrazol-3-yl]carbamoyl]-2-pyridyl]pyrrolidine-2-carboxylate

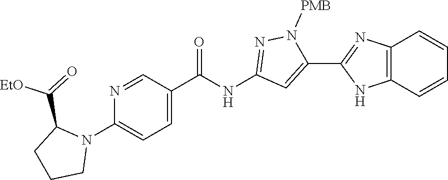

N-[5-(1H-Benzimidazol-2-yl)-1-[(4-methoxyphenyl)methyl]pyrazol-3-yl]-6-chloro-pyridine-3-carboxamide (L-1) (100 mg, 0.218 mmol), ethyl (2S)-pyrrolidine-2-carboxylate (45.7 μL, 0.327 mmol), and DIEA (74.6 mL, 0.436 mmol) were dissolved in DMSO (1 mL). The mixture was stirred at 100° C. for 42 h. Brine (25 mL) was added. The aq. phase was extracted with EtOAc (3×25 mL). The combined organic phases were washed with brine (25 mL), dried (MgSO₄), filtered, and concentrated. The residue was purified by silica gel chromatography eluting with hexanes and EtOAc (0-100%) to provide the title compound as a solid (46.2 mg, 38%). ¹H NMR (500 MHz, DMSO) δ 13.12 (s, 1H), 10.86 (s, 1H), 8.73 (s, 1H), 8.17 (d, J=8.6 Hz, 1H), 7.78-7.66 (m, 1H), 7.62-7.51 (m, 1H), 7.54 (s, 1H), 7.31-7.18 (m, 4H), 6.90-6.82 (m, 2H), 6.61-6.50 (m, 1H), 6.03 (s, 2H), 4.58 (d, J=7.0 Hz, 1H), 4.11 (dt, J=12.7, 6.2 Hz, 2H), 3.69 (s, 3H), 3.62-3.55 (m, 1H), 3.55-3.46 (m, 1H), 2.38-2.22 (m, 1H), 2.08-1.98 (m, 3H), 1.18 (t, J=7.1 Hz, 3H). m/z (ES⁺), [M+H]⁺ 566.7. HPLC (A05) t_{R}=2.59 min.

### Step 2: ethyl (2S)-1-[5-[[5-(1H-benzimidazol-2-yl)-1H-pyrazol-3-yl]carbamoyl]-2-pyridyl]pyrrolidine-2-carboxylate

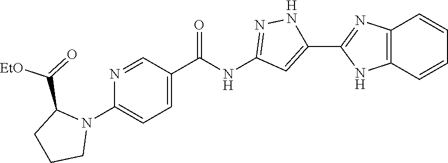

TFA (1 mL) was added to ethyl (2S)-1-[5-[[5-(1H-benzimidazol-2-yl)-1-[(4-methoxyphenyl)methyl]pyrazol-3-yl]carbamoyl]-2-pyridyl]pyrrolidine-2-carboxylate (45 mg, 0.0796 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 5 min. Water (25 mL) was added. The mixture was filtered, and the solid was washed with water (25 mL), acetone (25 mL), DCM (25 mL), and acetone (25 mL) and dried under high vacuum to provide the title compound as a solid (11.7 mg, 33%) . ¹H NMR (400 MHz, DMSO) δ 13.40 (br, 1H), 12.97 (br, 1H), 10.74 (br, 1H), 8.87-8.51 (m, 1H), 8.27-7.96 (m, 1H), 7.73-7.28 (m, 3H), 7.28-7.01 (m, 2H), 6.68-6.37 (m, 1H), 4.72-4.40 (m, 1H), 4.21-3.93 (m, 2H), 3.71-3.41 (m, 2H), 2.35-2.21 (m, 1H), 2.15-1.83 (m, 3H), 1.31-0.90 (m, 3H). m/z (ES⁺), [M+H]⁺ 446.5. HPLC (A05) t_{R}=2.19 min.

### Preparation of Compound A-59.

### Step 1: N-[5-(1H-benzimidazol-2-yl)-1-[(4-methoxyphenyl)methyl]pyrazol-3-yl]-6-(3-hydroxy-3-methyl-1-piperidyl)pyridine-3-carboxamide

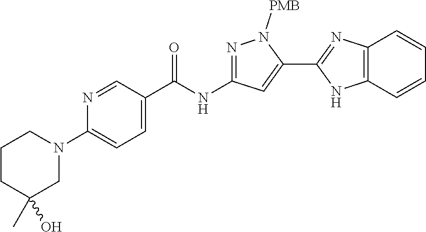

N-[5-(1H-Benzimidazol-2-yl)-1-[(4-methoxyphenyl)methyl]pyrazol-3-yl]-6-chloro-pyridine-3-carboxamide (L-1) (100 mg, 0.218 mmol), 3-methylpiperidin-3-ol (37.6 mg, 0.327 mmol), and DIEA (74.6 μL, 0.436 mmol) were dissolved in DMSO (1 mL). The mixture was stirred at 100° C. for 18 h. Water (25 mL) was added. The mixture was filtered, and the solid was purified by silica gel chromatography eluting with hexanes and EtOAc (0-100%) to provide the title compound as a solid (73.4 mg, 63%). ¹H NMR (400 MHz, DMSO) δ 13.12 (s, 1H), 10.81 (s, 1H), 8.75 (d, J=2.3 Hz, 1H), 8.09 (dd, J=9.1, 2.5 Hz, 1H), 7.72 (d, J=7.9 Hz, 1H), 7.55 (s, 1H), 7.55 (d, J=7.3 Hz, 1H), 7.32-7.26 (m, 1H), 7.26-7.20 (m, 3H), 6.90-6.79 (m, 3H), 6.03 (s, 2H), 4.44 (s, 1H), 3.91-3.80 (m, 1H), 3.69 (s, 3H), 3.59 (d, J=12.9 Hz, 1H), 3.45-3.36 (m, 2H), 1.81-1.68 (m, 1H), 1.64-1.54 (m, 2H), 1.52-1.42 (m, 1H), 1.12 (s, 3H). m/z (ES⁺), [M+H]⁺ 538.6. HPLC (A05) t_{R}=2.40 min.

### Step 2: N-[5-(1H-benzimidazol-2-yl)-1H-pyrazol-3-yl]-6-(3-hydroxy-3-methyl-1-piperidyl)pyridine-3-carboxamide

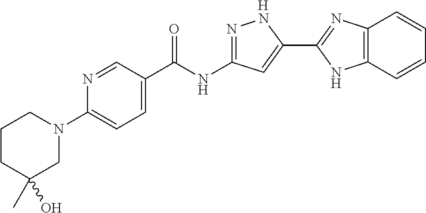

TFA (1 mL) was added to N-[5-(1H-benzimidazol-2-yl)-1-[(4-methoxyphenyl)methyl]pyrazol-3-yl]-6-(3-hydroxy-3-methyl-1-piperidyl)pyridine-3-carboxamide (70.9 mg, 0.132 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL) and dried under high vacuum to provide a 7:3 mixture of pyrazole tautomers of the title compound as a solid (40.7 mg, 74%). ¹H NMR (400 MHz, DMSO) δ 13.44 [13.09] (br, 1H), 13.02 [12.73] (s, 1H), 10.72 [10.86] (s, 1H), 8.79-8.70 [8.79-8.70] (m, 1H), 8.15-7.98 [8.15-7.98] (m, 1H), 7.75-7.60 [7.75-7.60] (m, 1H), 7.58-7.44 [7.58-7.44] (m, 1H), 7.41 [6.63] (s, 1H), 7.31-7.13 [7.31-7.13] (m, 2H), 6.97-6.78 [6.97-6.78] (m, 1H), 4.45 [4.45] (s, 1H), 3.93-3.79 [3.93-3.79] (m, 1H), 3.67-3.53 [3.67-3.53] (m, 1H), 3.52-3.38 [3.52-3.38] (m, 2H), 1.84-1.69 [1.84-1.69] (m, 1H), 1.67-1.55 [1.67-1.55] (m, 2H), 1.54-1.43 [1.54-1.43] (m, 1H), 1.13 [1.13] (s, 3H). m/z (ES⁺), [M+H]⁺ 418.5. HPLC (A05) t_{R}=1.99 min.

### Preparation of Compound A-60.

### Step 1: N-[5-(1H-benzimidazol-2-yl)-1-[(4-methoxyphenyl)methyl]pyrazol-3-yl]-6-[(3S,4S)-3,4-dihydroxypyrrolidin-1-yl]pyridine-3-carboxamide

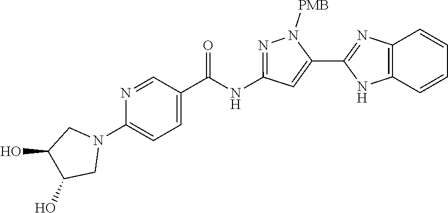

N-[5-(1H-Benzimidazol-2-yl)-1-[(4-methoxyphenyl)methyl]pyrazol-3-yl]-6-chloro-pyridine-3-carboxamide (L-1) (100 mg, 0.218 mmol), (3S,4S)-pyrrolidine-3,4-diol (33.7 mg, 0.327 mmol), and DIEA (74.6 μL, 0.436 mmol) were dissolved in DMSO (1 mL). The mixture was stirred at 100° C. for 18 h. Water (25 mL) was added. The mixture was filtered, and the solid was purified by silica gel chromatography eluting with hexanes and EtOAc (0-100%) and with DCM and MeOH (0-20%) to provide the title compound as a solid (72.5 mg, 63%). ¹H NMR (400 MHz, DMSO) δ 13.13 (s, 1H), 10.81 (s, 1H), 8.78 (d, J=2.5 Hz, 1H), 8.12 (dd, J=9.0, 2.5 Hz, 1H), 7.72 (d, J=7.4 Hz, 1H), 7.55 (s, 1H), 7.54 (d, J=5.1 Hz, 1H), 7.32-7.27 (m, 1H), 7.27-7.21 (m, 3H), 6.89-6.82 (m, 2H), 6.48 (d, J=8.9 Hz, 1H), 6.03 (s, 2H), 5.20-5.13 (m, 2H), 4.14-4.03 (m, 2H), 3.69 (s, 3H), 3.63-3.56 (m, 2H), 3.47-3.37 (m, 1H), 3.18 (d, J=5.2 Hz, 1H). m/z (ES⁺), [M+H]⁺ 526.6. HPLC (A05) t_{R}=2.15 min.

### Step 2: N-[5-(1H-benzimidazol-2-yl)-1H-pyrazol-3-yl]-6-[(3S,4S)-3,4-dihydroxy-pyrrolidin-1-yl]pyridine-3-carboxamide

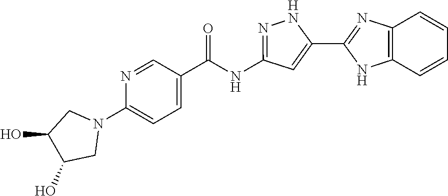

TFA (1 mL) was added to N-[5-(1H-benzimidazol-2-yl)-1-[(4-methoxyphenyl)methyl]pyrazol-3-yl]-6-[(3S,4S)-3,4-dihydroxypyrrolidin-1-yl]pyridine-3-carboxamide (70.3 mg, 0.134 mmol), and the mixture was stirred at 23° C. for 2 h and at 50° C. for 1 h. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL) and dried under high vacuum to provide a 5:3 mixture of pyrazole tautomers of the title compound as a solid (25.8 mg, 48%). ¹H NMR (500 MHz, DMSO) δ 13.43 [13.10] (m, 1H), 13.01 [12.72] (s, 1H), 10.71 [10.88] (s, 1H), 8.89-8.69 [8.89-8.69] (m, 1H), 8.21-8.03 [8.21-8.03] (m, 1H), 7.76-7.59 [7.76-7.59] (m, 1H), 7.57-7.45 [7.57-7.45] (m, 1H), 7.41 [6.63] (s, 1H), 7.31-7.13 [7.31-7.13] (m, 2H), 6.59-6.46 [6.59-6.46] (m, 1H), 5.23-5.11 [5.23-5.11] (m, 2H), 4.13-4.01 [4.13-4.01] (m, 2H), 3.69-3.56 [3.69-3.56] (m, 2H), 3.53-3.18 [3.53-3.18] (m, 2H). m/z (ES⁺), [M+H]⁺ 406.4. HPLC (A05) t_{R}=1.77 min.

### Preparation of Compound A-61.

### Step 1: N-[5-(1H-benzimidazol-2-yl)-1-[(4-methoxyphenyl)methyl]pyrazol-3-yl]-6-(2-ethoxyethylamino)pyridine-3-carboxamide

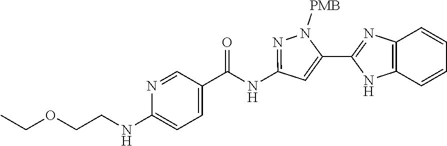

N-[5-(1H-Benzimidazol-2-yl)-1-[(4-methoxyphenyl)methyl]pyrazol-3-yl]-6-chloro-pyridine-3-carboxamide (L-1) (100 mg, 0.218 mmol), 2-ethoxyethanamine (29.1 mg, 0.327 mmol), and DIEA (74.6 μL, 0.436 mmol) were dissolved in DMSO (1 mL). The mixture was stirred at 100° C. for 18 h. Water (25 mL) was added. The mixture was filtered, and the solid was purified by silica gel chromatography eluting with hexanes and EtOAc (0-100%) to provide the title compound as a solid (76.5 mg, 67%). ¹H NMR (400 MHz, DMSO) δ 13.12 (s, 1H), 10.76 (s, 1H), 8.70 (d, J=2.4 Hz, 1H), 7.99 (dd, J=8.9, 2.6 Hz, 1H), 7.75-7.70 (m, 1H), 7.55 (d, J=6.2 Hz, 1H), 7.54 (s, 1H), 7.31-7.26 (m, 1H), 7.26-7.20 (m, 3H), 6.89-6.83 (m, 2H), 6.54 (d, J=8.4 Hz, 1H), 6.03 (s, 2H), 3.69 (s, 3H), 3.52-3.50 (m, 4H), 3.47 (q, J=7.0 Hz, 2H), 1.13 (t, J=7.0 Hz, 3H). m/z (ES⁺), [M+H]⁺ 512.5. HPLC (A05) t_{R}=2.41 min.

### Step 2: N-[5-(1H-benzimidazol-2-yl)-1H-pyrazol-3-yl]-6-(2-ethoxyethylamino)-pyridine-3-carboxamide

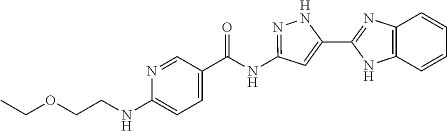

TFA (1 mL) was added to N-[5-(1H-benzimidazol-2-yl)-1-[(4-methoxyphenyl)methyl]pyrazol-3-yl]-6-(2-ethoxyethylamino)pyridine-3-carboxamide (21.5 mg, 0.0420 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL) and dried under high vacuum to provide a 2:1 mixture of pyrazole tautomers of the title compound as a solid (40.7 mg, 74%). ¹H NMR (400 MHz, DMSO) δ 13.43 [13.08] (s, 1H), 13.02 [12.74] (s, 1H), 10.67 [10.85] (s, 1H), 8.74-8.66 [8.74-8.66] (m, 1H), 8.06-7.91 [8.06-7.91] (m, 1H), 7.74-7.60 [7.74-7.60] (m, 1H), 7.58-7.44 [7.58-7.44] (m, 2H), 7.40 [6.58] (s, 1H), 7.31-7.12 [7.31-7.12 (m, 2H), 6.66-6.51 [6.66-6.51] (m, 1H), 3.62-3.42 [3.62-3.42] (m, 6H), 1.20-1.08 [1.20-1.08 (m, 3H). m/z (ES⁺), [M+H]⁺ 392.4. HPLC (A05) t_{R}=1.99 min.

### Preparation of Compound A-63.

### Step 1: N-[5-(1H-benzimidazol-2-yl)-1-[(4-methoxyphenyl)methyl]pyrazol-3-yl]-6-[[(1R)-2-hydroxy-1-methyl-ethyl]amino]pyridine-3-carboxamide

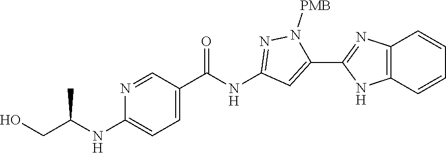

N-[5-(1H-Benzimidazol-2-yl)-1-[(4-methoxyphenyl)methyl]pyrazol-3-yl]-6-chloro-pyridine-3-carboxamide (L-1) (100 mg, 0.218 mmol), (2R)-2-aminopropan-1-ol (24.6 mg, 0.327 mmol), and DIEA (74.6 μL, 0.436 mmol) were dissolved in DMSO (1 mL). The mixture was stirred at 100° C. for 18 h. Water (25 mL) was added. The mixture was filtered, and the solid was purified by silica gel chromatography eluting with hexanes and EtOAc (0-100%) to provide the title compound as a solid (14.1 mg, 13%). ¹H NMR (400 MHz, DMSO) δ 13.11 (s, 1H), 10.74 (s, 1H), 8.68 (d, J=2.4 Hz, 1H), 7.96 (dd, J=8.9, 2.5 Hz, 1H), 7.71 (d, J=7.8 Hz, 1H), 7.54 (d, J=5.1 Hz, 2H), 7.30-7.20 (m, 4H), 6.95 (d, J=7.7 Hz, 1H), 6.88-6.83 (m, 2H), 6.50 (d, J=8.8 Hz, 1H), 6.02 (s, 2H), 4.74 (t, J=5.4 Hz, 1H), 3.68 (s, 3H), 3.48 (dt, J=10.2, 3.4 Hz, 1H), 3.29 (d, J=5.9 Hz, 1H), 1.13 (d, J=6.6 Hz, 3H). m/z (ES⁺), [M+H]⁺ 498.5. HPLC (A05) t_{R}=2.25 min.

### Step 2: N-[5-(1H-benzimidazol-2-yl)-1H-pyrazol-3-yl]-6-[[(1R)-2-hydroxy-1-methyl-ethyl]amino]pyridine-3-carboxamide

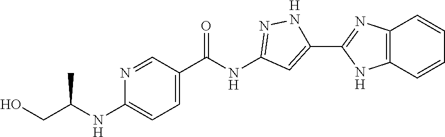

TFA (2 mL) was added to N-[5-(1H-benzimidazol-2-yl)-1-[(4-methoxyphenyl)methyl]pyrazol-3-yl]-6-[[(1R)-2-hydroxy-1-methyl-ethyl]amino]pyridine-3-carboxamide (14 mg, 0.0281 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) and dried under high vacuum to provide a 2:1 mixture of pyrazole tautomers of the title compound as a solid (8.30 mg, 78%). ¹H NMR (500 MHz, DMSO) δ 13.40 [13.05] (s, 1H), 12.99 [12.72] (s, 1H), [10.80] 10.63 (s, 1H), 8.69 [8.67] (d, J=2.1 Hz, 1H), 7.97 [7.93] (dd, J=8.8, 2.3 Hz, 1H), 7.67 [7.61] (d, J=7.6 Hz, 1H), 7.53 [7.46] (d, J=7.4 Hz, 1H), 7.39 [6.60] (d, J=1.5 Hz, 1H), 7.26-7.14 [7.26-7.14] (m, 2H), [7.10] 6.94 (d, J=7.8 Hz, 1H), [6.57] 6.52 (d, J=9.0 Hz, 1H), 4.74 [4.74] (t, J=5.1 Hz, 1H), 4.04 [4.04] (s, 1H), 3.49 [3.49] (dt, J=10.1, 5.1 Hz, 1H), 3.36-3.32 [3.36-3.32] (m, 1H), 1.14 [1.14] (d, J=6.6 Hz, 3H). m/z (ES⁺), [M+H]⁺ 378.4. HPLC (A05) t_{R}=1.85 min.

### Preparation of Compound A-64.

### Step 1: N-[5-(1H-benzimidazol-2-yl)-1-[(4-methoxyphenyl)methyl]pyrazol-3-yl]-6-(3-hydroxyazetidin-1-yl)pyridine-3-carboxamide

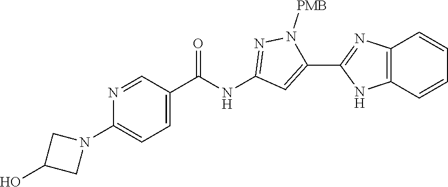

N-[5-(1H-Benzimidazol-2-yl)-1-[(4-methoxyphenyl)methyl]pyrazol-3-yl]-6-chloro-pyridine-3-carboxamide (L-1) (100 mg, 0.218 mmol), azetidin-3-ol hydrochloride (35.8 mg, 0.327 mmol), and DIEA (0.149 mL, 0.872 mmol) were dissolved in DMSO (1 mL). The mixture was stirred at 100° C. for 18 h. Water (25 mL) was added. The mixture was filtered, and the solid was purified by silica gel chromatography eluting with hexanes and EtOAc (0-100%) to provide the title compound as a solid (56.2 mg, 52%). ¹H NMR (400 MHz, DMSO) δ 13.11 (s, 1H), 10.85 (s, 1H), 8.75 (d, J=1.8 Hz, 1H), 8.11 (dd, J=8.8, 2.4 Hz, 1H), 7.72 (d, J=7.4 Hz, 1H), 7.56-7.51 (m, 2H), 7.31-7.19 (m, 4H), 6.88-6.82 (m, 2H), 6.39 (d, J=8.8 Hz, 1H), 6.02 (s, 2H), 5.72 (d, J=6.5 Hz, 1H), 4.60 (tdd, J=11.1, 6.7, 4.6 Hz, 1H), 4.27-4.20 (m, 2H), 3.76 (dd, J=9.9, 4.6 Hz, 2H), 3.68 (s, 3H). m/z (ES⁺), [M+H]⁺ 496.9. HPLC (A05) t_{R}=2.23 min.

### Step 2: N-[5-(1H-benzimidazol-2-yl)-1H-pyrazol-3-yl]-6-(3-hydroxyazetidin-1-yl)pyridine-3-carboxamide

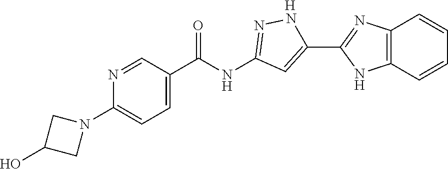

TFA (2 mL) was added to N-[5-(1H-benzimidazol-2-yl)-1-[(4-methoxyphenyl)methyl]pyrazol-3-yl]-6-(3-hydroxyazetidin-1-yl)pyridine-3-carboxamide (48 mg, 0.0969 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) and dried under high vacuum to provide the title compound as a solid (22.3 mg, 61%). ¹H NMR (400 MHz, DMSO) δ 13.44 (s, 1H), 13.02 (s, 1H), 10.77 (s, 1H), 8.76 (s, 1H), 8.11 (d, J=9.4 Hz, 1H), 7.72-7.37 (m, 2H), 7.22 (s, 2H), 6.94-6.58 (m, 1H), 6.42 (d, J=9.2 Hz, 1H), 5.84-5.63 (m, 1H), 4.67-4.57 (m, 1H), 4.33-4.20 (m, 2H), 3.78 (dd, J=9.1, 4.2 Hz, 2H). m/z (ES⁺), [M+H]⁺ 376.4. HPLC (A05) t_{R}=1.83 min.

### Preparation of Compound A-65.

### Step 1: N-[5-(1H-benzimidazol-2-yl)-1-[(4-methoxyphenyl)methyl]pyrazol-3-yl]-6-(3-methoxyazetidin-1-yl)pyridine-3-carboxamide

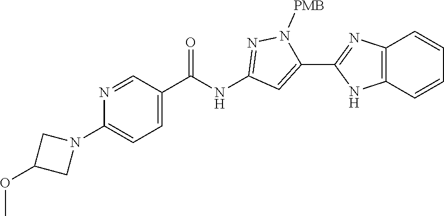

N-[5-(1H-Benzimidazol-2-yl)-1-[(4-methoxyphenyl)methyl]pyrazol-3-yl]-6-chloro-pyridine-3-carboxamide (L-1) (100 mg, 0.218 mmol), 3-methoxyazetidine hydrochloride (40.4 mg, 0.327 mmol), and DIEA (149 μL, 0.872 mmol) were dissolved in DMSO (1 mL). The mixture was stirred at 100° C. for 18 h. Water (25 mL) was added. The mixture was filtered, and the solid was purified by silica gel chromatography eluting with hexanes and EtOAc (0-100%) to provide the title compound as a solid (64.1 mg, 58%). ¹H NMR (400 MHz, DMSO) δ 13.12 (s, 1H), 10.87 (s, 1H), 8.76 (dd, J=2.4, 0.7 Hz, 1H), 8.13 (dd, J=8.8, 2.4 Hz, 1H), 7.72 (d, J=8.0 Hz, 1H), 7.56-7.51 (m, 2H), 7.31-7.20 (m, 4H), 6.87-6.83 (m, 2H), 6.42 (d, J=8.8 Hz, 1H), 6.02 (s, 2H), 4.35 (td, J=6.3, 3.2 Hz, 1H), 4.23 (ddd, J=9.1, 6.1, 0.7 Hz, 2H), 3.85 (dd, J=10.4, 3.9 Hz, 2H), 3.68 (s, 3H), 3.26 (s, 3H). m/z (ES⁺), [M+H]⁺ 510.2. HPLC (A05) t_{R}=2.39 min.

### Step 2: N-[5-(1H-benzimidazol-2-yl)-1H-pyrazol-3-yl]-6-(3-methoxyazetidin-1-yl)pyridine-3-carboxamide

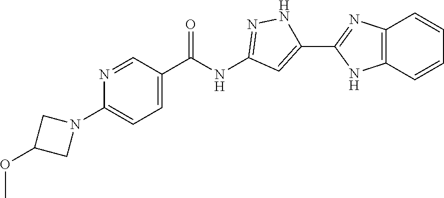 [SMILES: COC1CN(c2ccc(C(=O)Nc3cc(-c4nc5ccccc5[nH]4)[nH]n3)cn2)C1]

TFA (2 mL) was added to N-[5-(1H-benzimidazol-2-yl)-1-[(4-methoxyphenyl)methyl]pyrazol-3-yl]-6-(3-methoxyazetidin-1-yl)pyridine-3-carboxamide (64 mg, 0.126 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) and dried under high vacuum to provide a 3:1 mixture of pyrazole tautomers of the title compound as a solid (42 mg, 86%). ¹H NMR (400 MHz, DMSO) δ 13.44 [13.10] (s, 1H), 13.00 [12.72] (s, 1H), [10.92] 10.78 (s, 1H), 8.76 [8.76] (s, 1H), 8.13 [8.13] (d, J=10.4 Hz, 1H), 7.71-7.58 [7.71-7.58] (m, 1H), 7.49 [7.49] (dd, J=26.8, 5.5 Hz, 1H), 7.40 [6.62] (s, 1H), 7.24-7.14 [7.24-7.14] (m, 2H), 6.44 [6.44] (d, J=9.2 Hz, 1H), 4.42-4.31 [4.42-4.31] (m, 1H), 4.24 [4.24] (t, J=6.7 Hz, 2H), 3.86 [3.86] (d, J=9.4 Hz, 2H), 3.27 [3.27] (s, 3H). m/z (ES⁺), [M+H]⁺ 390.8. HPLC (A05) t_{R}=1.97 min.

### Preparation of Compound A-66.

### Step 1: N-[5-(1H-benzimidazol-2-yl)-1-[(4-methoxyphenyl)methyl]pyrazol-3-yl]-6-(3-hydroxy-1-piperidyl)pyridine-3-carboxamide

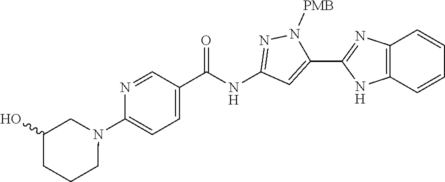

N-[5-(1H-Benzimidazol-2-yl)-1-[(4-methoxyphenyl)methyl]pyrazol-3-yl]-6-chloro-pyridine-3-carboxamide (L-1) (100 mg, 0.218 mmol), piperidin-3-ol (33.1 mg, 0.327 mmol), and DIEA (74.6 μL, 0.436 mmol) were dissolved in DMSO (1 mL). The mixture was stirred at 100° C. for 18 h. Water (25 mL) was added. The mixture was filtered, and the solid was purified by silica gel chromatography eluting with hexanes and EtOAc (0-100%) to provide the title compound as a solid (76.5 mg, 67%). ¹H NMR (400 MHz, DMSO) δ 13.13 (s, 1H), 10.84 (s, 1H), 8.77 (d, J=2.2 Hz, 1H), 8.11 (dd, J=9.1, 2.5 Hz, 1H), 7.78-7.66 (m, 1H), 7.55 (s, 1H), 7.58-7.51 (m, 1H), 7.31-7.21 (m, 4H), 6.89-6.81 (m, 3H), 6.03 (s, 2H), 4.89 (d, J=4.2 Hz, 1H), 4.30-4.21 (m, 1H), 4.09-3.96 (m, 1H), 3.69 (s, 3H), 3.55-3.41 (m, 1H), 3.18-3.04 (m, 1H), 2.90 (dd, J=12.7, 8.9 Hz, 1H), 1.97-1.87 (m, 1H), 1.79-1.66 (m, 1H), 1.49-1.33 (m, 2H). m/z (ES⁺), [M+H]⁺ 524.6. HPLC (A05) t_{R}=2.34 min.

### Step 2: N-[5-(1H-benzimidazol-2-yl)-1H-pyrazol-3-yl]-6-(3-hydroxy-1-piperidyl)pyridine-3-carboxamide

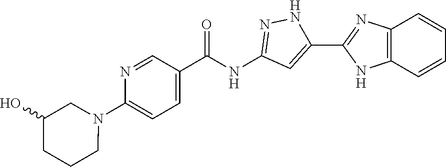 [SMILES: O=C(Nc1cc(-c2nc3ccccc3[nH]2)[nH]n1)c1ccc(N2CCCC(O)C2)nc1]

TFA (1 mL) was added to N-[5-(1H-benzimidazol-2-yl)-1-[(4-methoxyphenyl)methyl]pyrazol-3-yl]-6-(3-hydroxy-1-piperidyl)pyridine-3-carboxamide (71.2 mg, 0.136 mmol), and the mixture was stirred at 23° C. for 2 h and at 50° C. for 1 h. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL) and dried under high vacuum. The solid was purified by Waters HPLC (BEH C18 30×100) eluting with water (10 mM (NH₄)(HCO₂)) and MeCN (27-37%) to provide a 2:1 mixture of pyrazole tautomers of the title compound as a solid (6.7 mg, 12%). ¹H NMR (400 MHz, DMSO) δ 13.44 [13.09] (s, 1H), 13.02 [12.74] (s, 1H), 10.74 [10.89] (s, 1H), 8.82-8.72 [8.82-8.72] (m, 1H), 8.17-8.04 [8.17-8.04] (m, 1H), 7.69-7.65 [7.65-7.60] (m, 1H), 7.57-7.51 [7.49-7.44] (m, 1H), 7.41 [6.63] (s, 1H), 7.28-7.21 [7.21-7.12] (m, 2H), 6.90-6.82 [6.97-6.90] (m, 1H), 4.95-4.86 [4.95-4.86] (m, 1H), 4.32-4.21 [4.32-4.21] (m, 1H), 4.11-3.97 [4.11-3.97] (m, 1H), 3.59-3.43 [3.59-3.43] (m, 1H), 3.19-3.07 [3.19-3.07] (m, 1H), 2.98-2.85 [2.98-2.85] (m, 1H), 1.99-1.85 [1.99-1.85] (m, 1H), 1.81-1.68 [1.81-1.68] (m, 1H), 1.50-1.36 [1.50-1.36] (m, 2H). m/z (ES⁺), [M+H]⁺ 404.4. HPLC (A05) t_{R}=1.95 min.

### Preparation of Compound A-67.

### Step 1: N-[5-(1H-benzimidazol-2-yl)-1-[(4-methoxyphenyl)methyl]pyrazol-3-yl]-6-[4-(hydroxymethyl)-1-piperidyl]pyridine-3-carboxamide

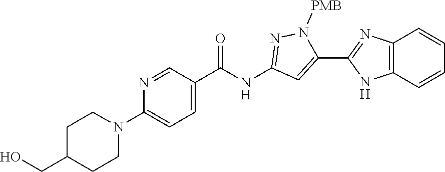

N-[5-(1H-Benzimidazol-2-yl)-1-[(4-methoxyphenyl)methyl]pyrazol-3-yl]-6-chloro-pyridine-3-carboxamide (L-1) (100 mg, 0.218 mmol), 4-piperidylmethanol (37.6 mg, 0.327 mmol), and DIEA (74.6 μL, 0.436 mmol) were dissolved in DMSO (1 mL). The mixture was stirred at 100° C. for 18 h. Water (25 mL) was added. The mixture was filtered, and the solid was purified by silica gel chromatography eluting with hexanes and EtOAc (0-100%) to provide the title compound as a solid (77.2 mg, 66%). ¹H NMR (400 MHz, DMSO) δ 13.13 (s, 1H), 10.84 (s, 1H), 8.78 (d, J=2.7 Hz, 1H), 8.12 (dd, J=9.1, 2.5 Hz, 1H), 7.73 (d, J=7.9 Hz, 1H), 7.55 (s, 1H), 7.54 (d, J=5.4 Hz, 1H), 7.32-7.26 (m, 1H), 7.26-7.21 (m, 3H), 6.89-6.83 (m, 3H), 6.03 (s, 2H), 4.51-4.40 (m, 3H), 3.69 (s, 3H), 3.28 (t, J=5.7 Hz, 2H), 2.95-2.81 (m, 2H), 1.80-1.60 (m, 3H), 1.18-1.03 (m, 2H). m/z (ES⁺), [M+H]⁺ 538.6. HPLC (A05) t_{R}=2.35 min.

### Step 2: N-[5-(1H-benzimidazol-2-yl)-1H-pyrazol-3-yl]-6-[4-(hydroxymethyl)-1-piperidyl]pyridine-3-carboxamide

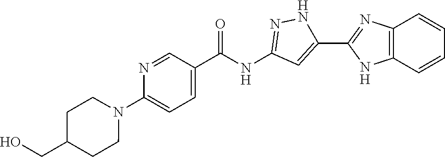 [SMILES: O=C(Nc1cc(-c2nc3ccccc3[nH]2)[nH]n1)c1ccc(N2CCC(CO)CC2)nc1]

TFA (1 mL) was added to N-[5-(1H-benzimidazol-2-yl)-1-[(4-methoxyphenyl)methyl]pyrazol-3-yl]-6-[4-(hydroxymethyl)-1-piperidyl]pyridine-3-carboxamide (55 mg, 0.102 mmol), and the mixture was stirred at 23° C. for 20 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL) and dried under high vacuum to provide the title compound as a solid (22.5 mg, 53%). ¹H NMR (400 MHz, DMSO) δ 13.44 (s, 1H), 12.98 (s, 1H), 10.79 (s, 1H), 8.92-8.64 (m, 1H), 8.23-7.99 (m, 1H), 7.70-7.48 (m, 2H), 7.33-7.14 (m, 2H), 6.94-6.84 (m, 2H), 4.63-4.29 (m, 3H), 3.04-2.76 (m, 3H), 1.89-1.51 (m, 4H), 1.28-0.94 (m, 2H). m/z (ES+), [M+H]⁺ 418.4. HPLC (A05) t_{R}=1.97 min.

### Preparation of Compound A-68.

### Step 1: N-[5-(1H-benzimidazol-2-yl)-1-[(4-methoxyphenyl)methyl]pyrazol-3-yl]-6-[3-(hydroxymethyl)-1-piperidyl]pyridine-3-carboxamide

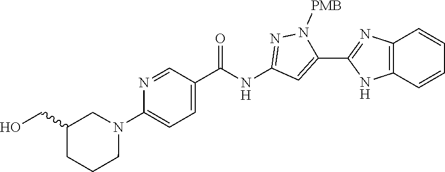

N-[5-(1H-Benzimidazol-2-yl)-1-[(4-methoxyphenyl)methyl]pyrazol-3-yl]-6-chloro-pyridine-3-carboxamide (L-1) (100 mg, 0.218 mmol), 3-piperidylmethanol (37.6 mg, 0.327 mmol), and DIEA (74.6 μL, 0.436 mmol) were dissolved in DMSO (1 mL). The mixture was stirred at 100° C. for 18 h. Water (25 mL) was added. The mixture was filtered, and the solid was purified by silica gel chromatography eluting with hexanes and EtOAc (0-100%) to provide the title compound as a solid (81.9 mg, 70%). ¹H NMR (400 MHz, DMSO) δ 13.13 (s, 1H), 10.83 (s, 1H), 8.78 (d, J=2.1 Hz, 1H), 8.12 (dd, J=9.1, 2.5 Hz, 1H), 7.76-7.69 (m, 1H), 7.55 (s, 1H), 7.57-7.51 (m, 1H), 7.32-7.18 (m, J=8.8 Hz, 4H), 6.91-6.79 (m, 3H), 6.03 (s, 2H), 4.59 (t, J=5.3 Hz, 1H), 4.48-4.37 (m, 1H), 4.35-4.23 (m, 1H), 3.69 (s, 3H), 3.37 (dd, J=10.6, 5.4 Hz, 1H), 3.29 (d, J=10.8 Hz, 1H), 2.99-2.91 (m, 1H), 2.76-2.65 (m, 1H), 1.81-1.67 (m, 2H), 1.66-1.52 (m, 1H), 1.49-1.37 (m, 1H), 1.31-1.21 (m, 1H). m/z (ES⁺), [M+H]⁺ 538.6. HPLC (A05) t_{R}=2.40 min.

### Step 2: N-[5-(1H-benzimidazol-2-yl)-1H-pyrazol-3-yl]-6-[3-(hydroxymethyl)-1-piperidyl]pyridine-3-carboxamide

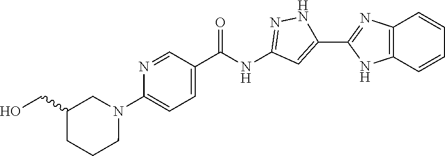 [SMILES: O=C(Nc1cc(-c2nc3ccccc3[nH]2)[nH]n1)c1ccc(N2CCCC(CO)C2)nc1]

TFA (1 mL) was added to N-[5-(1H-benzimidazol-2-yl)-1-[(4-methoxyphenyl)methyl]pyrazol-3-yl]-6-[3-(hydroxymethyl)-1-piperidyl]pyridine-3-carboxamide (60 mg, 0.112 mmol), and the mixture was stirred at 23° C. for 20 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL) and dried under high vacuum to provide a 2:1 mixture of pyrazole tautomers of the title compound as a solid (34 mg, 73%). ¹H NMR (400 MHz, DMSO) δ 13.44 [13.09] (s, 1H), 13.02 [12.74] (s, 1H), 10.74 [10.89] (s, 1H), 8.82-8.73 [8.82-8.73] (m, 1H), 8.17-8.04 [8.17-8.04] (m, 1H), 7.73-7.59 [7.73-7.59] (m, 1H), 7.59-7.47 [7.59-7.47] (m, 1H), 7.41 [6.64] (s, 1H), 7.30-7.10 [7.30-7.10] (m, 2H), 6.94-6.78 [6.94-6.78] (m, 1H), 4.67-4.53 [4.67-4.53] (m, 1H), 4.49-4.38 [4.49-4.38] (m, 1H), 4.37-4.24 [4.37-4.24] (m, 1H), 3.44-3.35 [3.44-3.35] (m, 1H), 3.27-3.20 [3.27-3.20] (m, 1H), 3.03-2.88 [3.03-2.88] (m, 1H), 2.80-2.66 [2.80-2.66] (m, 1H), 1.84-1.67 [1.84-1.67] (m, 2H), 1.67-1.56 [1.67-1.56] (m, 1H), 1.52-1.35 [1.52-1.35] (m, 1H), 1.32-1.14 [1.32-1.14] (m, 1H). m/z (ES⁺), [M+H]⁺ 418.4. HPLC (A05) t_{R}=2.02 min.

### Preparation of Compound A-69.

### Step 1: N-[5-(1H-benzimidazol-2-yl)-1-[(4-methoxyphenyl)methyl]pyrazol-3-yl]-6-(3-oxopiperazin-1-yl)pyridine-3-carboxamide

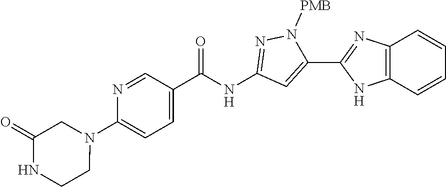

N-[5-(1H-Benzimidazol-2-yl)-1-[(4-methoxyphenyl)methyl]pyrazol-3-yl]-6-chloro-pyridine-3-carboxamide (L-1) (100 mg, 0.218 mmol), piperazin-2-one (37.6 mg, 0.376 mmol), and DIEA (74.6 μL, 0.436 mmol) were dissolved in DMSO (1 mL). The mixture was stirred at 100° C. for 18 h. Water (25 mL) was added. The mixture was filtered, and the solid was purified by silica gel chromatography eluting with DCM and MeOH (0-5%) to provide the title compound as a solid (48.2 mg, 42%). ¹H NMR (500 MHz, DMSO) δ 13.13 (s, 1H), 10.93 (s, 1H), 8.83 (d, J=2.4 Hz, 1H), 8.20 (dd, J=9.0, 2.5 Hz, 1H), 8.18-8.13 (m, 1H), 7.73 (d, J=8.0 Hz, 1H), 7.56 (s, 1H), 7.55 (d, J=9.1 Hz, 1H), 7.31-7.26 (m, 1H), 7.27-7.21 (m, 3H), 6.90-6.82 (m, 3H), 6.04 (s, 2H), 4.12 (s, 2H), 3.87-3.81 (m, 2H), 3.69 (s, 3H), 3.33-3.29 (m, 2H). m/z (ES⁺), [M+H]⁺ 523.5. HPLC (A05) t_{R}=2.22 min.

### Step 2: N-[5-(1H-benzimidazol-2-yl)-1H-pyrazol-3-yl]-6-(3-oxopiperazin-1-yl)pyridine-3-carboxamide

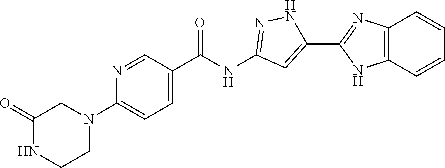 [SMILES: O=C1CN(c2ccc(C(=O)Nc3cc(-c4nc5ccccc5[nH]4)[nH]n3)cn2)CCN1]

TFA (1 mL) was added to N-[5-(1H-benzimidazol-2-yl)-1-[(4-methoxyphenyl)methyl]pyrazol-3-yl]-6-(3-oxopiperazin-1-yl)pyridine-3-carboxamide (47 mg, 0.0899 mmol), and the mixture was stirred at 23° C. for 45 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL) and dried under high vacuum to provide a 2:1 mixture of pyrazole tautomers of the title compound as a solid (34.9 mg, 96%). ¹H NMR (400 MHz, DMSO) δ 13.47 [13.13] (s, 1H), 13.03 [12.77] (s, 1H), 10.84 [10.97] (s, 1H), 8.93-8.74 [8.93-8.74] (m, 1H), 8.33-8.07 [8.33-8.07] (m, 2H), 7.77-7.59 [7.77-7.59] (m, 1H), 7.60-7.49 [7.60-7.49] (m, 1H), 7.44 [6.65] (s, 1H), 7.33-7.09 [7.33-7.09] (m, 2H), 7.03-6.80 [7.03-6.80] (m, 1H), 4.26-4.03 [4.26-4.03] (m, 2H), 3.95-3.75 [3.95-3.75] (m, 2H), 2.19-1.98 [2.19-1.98] (m, 2H). m/z (ES⁺), [M+H]⁺ 403.4. HPLC (A05) t_{R}=1.87 min.

### Preparation of Compound A-73.

### Step 1: N-[5-(1H-benzimidazol-2-yl)-1-[(4-methoxyphenyl)methyl]pyrazol-3-yl]-6-[[(1S)-2-methoxy-1-methyl-ethyl]amino]pyridine-3-carboxamide

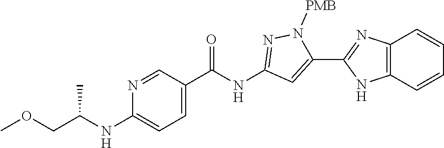

N-[5-(1H-Benzimidazol-2-yl)-1-[(4-methoxyphenyl)methyl]pyrazol-3-yl]-6-chloro-pyridine-3-carboxamide (L-1) (100 mg, 0.218 mmol), (2S)-1-methoxypropan-2-amine (29.1 mg, 0.327 mmol), and DIEA (74.6 μL, 0.436 mmol) were dissolved in DMSO (1 mL). The mixture was stirred at 100° C. for 18 h. Water (25 mL) was added. The mixture was filtered, and the solid was purified by silica gel chromatography eluting with hexanes and EtOAc (0-100%) to provide the title compound as a solid (19.1 mg, 17%). ¹H NMR (400 MHz, DMSO) δ 13.11 (s, 1H), 10.74 (s, 1H), 8.69 (d, J=2.4 Hz, 1H), 7.96 (dd, J=8.9, 2.4 Hz, 1H), 7.71 (d, J=7.6 Hz, 1H), 7.55-7.51 (m, 2H), 7.31-7.20 (m, 4H), 7.05 (d, J=8.0 Hz, 1H), 6.87-6.83 (m, 2H), 6.50 (d, J=8.8 Hz, 1H), 6.02 (s, 2H), 4.28-4.17 (m, 1H), 3.68 (s, 3H), 3.40 (dd, J=9.3, 5.4 Hz, 1H), 3.28-3.22 (m, 4H), 1.14 (d, J=6.7 Hz, 3H). m/z (ES⁺), [M+H]⁺ 512.3. HPLC (A05) t_{R}=2.42 min.

### Step 2: N-[5-(1H-benzimidazol-2-yl)-1H-pyrazol-3-yl]-6-[[(1S)-2-methoxy-1-methyl-ethyl]amino]pyridine-3-carboxamide

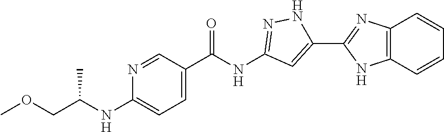 [SMILES: COC[C@H](C)Nc1ccc(C(=O)Nc2cc(-c3nc4ccccc4[nH]3)[nH]n2)cn1]

TFA (2 mL) was added to N-[5-(1H-benzimidazol-2-yl)-1-[(4-methoxyphenyl)methyl]pyrazol-3-yl]-6-[[(1S)-2-methoxy-1-methyl-ethyl]amino]pyridine-3-carboxamide (19 mg, 0.0371 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 5 min. The residue was purified by preparative HPLC (BEH C18 30×150) eluting with water (10 mM (NH₄)(HCO₃)) and MeCN (27-47%) to provide the title compound as a solid (2.7 mg, 19%). ¹H NMR (500 MHz, DMSO) δ 13.40 (s, 1H), 12.99 (s, 1H), 10.64 (s, 1H), 8.69 (s, 1H), 7.95 (s, 1H), 7.65 (s, 1H), 7.57-7.45 (m, 1H), 7.21 (s, 2H), 7.03 (s, 1H), 6.51 (s, 1H), 4.24 (d, J=2.7 Hz, 1H), 3.41 (dd, J=9.4, 5.5 Hz, 1H), 3.29-3.27 (m, 4H), 1.15 (d, J=6.7 Hz, 3H). m/z (ES⁺), [M+H]⁺ 392.4. HPLC (A05) t_{R}=1.99 min.

### Preparation of Compound A-80.

### Step 1: N-[5-(5-fluoro-1H-benzimidazol-2-yl)-1-[(4-methoxyphenyl)methyl]pyrazol-3-yl]-6-[4-(hydroxymethyl)-1-piperidyl]pyridine-3-carboxamide

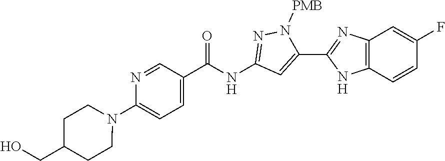

6-Chloro-N-[5-(5-fluoro-1H-benzimidazol-2-yl)-1-[(4-methoxyphenyl)methyl]pyrazol-3-yl]pyridine-3-carboxamide (L-2) (75 mg, 0.157 mmol), 4-piperidylmethanol (27.2 mg, 0.236 mmol), and DIEA (82.2 μL, 0.472 mmol) were dissolved in DMSO (1 mL). The mixture was stirred at 100° C. for 18 h. Water (10 mL) was added. The mixture was filtered, and the solid was purified by silica gel chromatography eluting with hexanes and EtOAc (0-100%) to provide the title compound as a solid (60.5 mg, 69%). ¹H NMR (500 MHz, DMSO) δ 13.27 (d, J=5.3 Hz, 1H), 10.84 (d, J=3.8 Hz, 1H), 8.77 (d, J=2.4 Hz, 1H), 8.11 (dd, J=9.1, 2.5 Hz, 1H), 7.80-7.68 [7.56-7.52] (m, 1H), 7.55 (d, J=1.8 Hz, 1H), 7.56-7.52 [7.37-7.31] (m, 1H), 7.26-7.19 (m, 2H), 7.19-7.12 [7.12-7.06] (m, 1H), 6.90-6.83 (m, 3H), 6.00 (d, J=3.2 Hz, 2H), 4.49 (t, J=5.3 Hz, 1H), 4.47-4.42 (m, 2H), 3.70 (s, 3H), 3.28 (t, J=5.7 Hz, 2H), 2.94-2.83 (m, 2H), 1.79-1.71 (m, 2H), 1.71-1.63 (m, 1H), 1.19-1.05 (m, 2H). m/z (ES⁺), [M+H]⁺ 556.6. HPLC (A05) t_{R}=2.41 min.

### Step 2: N-[5-(5-fluoro-1H-benzimidazol-2-yl)-1H-pyrazol-3-yl]-6-[4-(hydroxymethyl)-1-piperidyl]pyridine-3-carboxamide

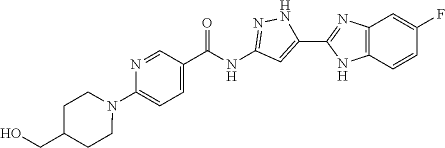 [SMILES: O=C(Nc1cc(-c2nc3cc(F)ccc3[nH]2)[nH]n1)c1ccc(N2CCC(CO)CC2)nc1]

TFA (1 mL) was added to N-[5-(5-fluoro-1H-benzimidazol-2-yl)-1-[(4-methoxyphenyl)methyl]pyrazol-3-yl]-6-[4-(hydroxymethyl)-1-piperidyl]pyridine-3-carboxamide (55 mg, 0.0990 mmol), and the mixture was stirred at 23° C. for 30 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL) and dried under high vacuum to provide a 5:3 mixture of pyrazole tautomers of the title compound as a solid (27.5 mg, 64%). ¹H NMR (500 MHz, DMSO) δ 13.45 [13.15] (d, J=21.2 Hz, 1H), 13.15 [12.86] (s, 1H), 10.89 [10.74] (s, 1H), 8.78 (s, 1H), 8.19-8.04 (m, 1H), 7.70-7.50 (m, 1H), 7.43-7.40 (m, 1H), 7.41 [6.63] (s, 1H), 7.24-6.99 (m, 1H), 6.97-6.83 (m, 1H), 4.57-4.39 (m, J=4.9 Hz, 3H), 3.34-3.25 (m, 2H), 2.99-2.83 (m, 2H), 1.86-1.59 (m, J=12.6 Hz, 3H), 1.19-1.05 (m, J=11.3 Hz, 2H). ¹⁹F NMR (471 MHz, DMSO) δ −119.24 [−120.10], −121.22 [−122.04]. m/z (ES⁺), [M+H]⁺ 436.4. HPLC (A05) t_{R}=2.03 min.

### Preparation of Compound A-81.

### Step 1: N-[5-(5-fluoro-1H-benzimidazol-2-yl)-1-[(4-methoxyphenyl)-methyl]pyrazol-3-yl]-6-(4-hydroxy-1-piperidyl)pyridine-3-carboxamide

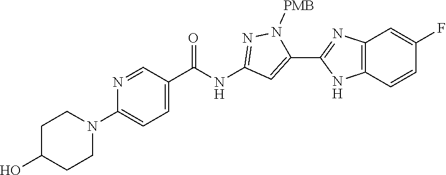

6-Chloro-N-[5-(5-fluoro-1H-benzimidazol-2-yl)-1-[(4-methoxyphenyl)methyl]pyrazol-3-yl]pyridine-3-carboxamide (L-2) (75 mg, 0.157 mmol), piperidin-4-ol (23.9 mg, 0.236 mmol), and DIEA (82.2 μL, 0.472 mmol) were dissolved in DMSO (1 mL). The mixture was stirred at 100° C. for 18 h. Water (10 mL) was added. The mixture was filtered, and the solid was purified by silica gel chromatography eluting with hexanes and EtOAc (0-100%) to provide the title compound as a solid (58.1 mg, 68%). ¹H NMR (500 MHz, DMSO) δ 13.27 (d, J=5.7 Hz, 1H), 10.85 (d, J=4.1 Hz, 1H), 8.78 (d, J=2.7 Hz, 1H), 8.12 (dd, J=9.1, 2.5 Hz, 1H), 7.73 [7.55] (dd, J=8.7, 4.9 Hz, 1H), 7.55 (d, J=2.0 Hz, 1H), 7.54-7.52 [7.33] (dd, J=8.7, 2.5 Hz, 1H), 7.26-7.20 (m, 2H), 7.18-7.12 [7.12-7.07] (m, 1H), 6.91-6.84 (m, 3H), 6.00 (d, J=3.4 Hz, 2H), 4.73 (d, J=4.3 Hz, 1H), 4.15-4.05 (m, 2H), 3.81-3.72 (m, 1H), 3.70 (s, 3H), 3.28-3.20 (m, 2H), 1.85-1.73 (m, 2H), 1.42-1.30 (m, J=9.4 Hz, 2H). m/z (ES⁺), [M+H]⁺ 542.5. HPLC (A05) t_{R}=2.36 min.

### Step 2: N-[5-(5-fluoro-1H-benzimidazol-2-yl)-1H-pyrazol-3-yl]-6-(4-hydroxy-1-piperidyl)pyridine-3-carboxamide

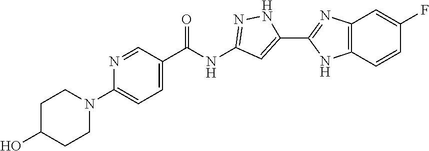 [SMILES: O=C(Nc1cc(-c2nc3cc(F)ccc3[nH]2)[nH]n1)c1ccc(N2CCC(O)CC2)nc1]

TFA (1 mL) was added to N-[5-(5-fluoro-1H-benzimidazol-2-yl)-1-[(4-methoxyphenyl)methyl]pyrazol-3-yl]-6-(4-hydroxy-1-piperidyl)pyridine-3-carboxamide (52.3 mg, 0.0966 mmol), and the mixture was stirred at 23° C. for 30 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL) and dried under high vacuum to provide a 3:2 mixture of pyrazole tautomers of the title compound as a solid (30.3 mg, 75%). ¹H NMR (500 MHz, DMSO) δ 13.46 [13.15] (d, J=16.6 Hz, 1H), 13.15 [12.85] (s, 1H), 10.76 [10.90] (s, 1H), 8.78 (s, 1H), 8.17-8.04 (m, 1H), 7.72-7.50 (m, 1H), 7.42 [6.62] (s, 1H), 7.48-7.31 (m, 1H), 7.24-7.00 (m, 1H), 6.99-6.84 (m, 1H), 4.80-4.68 (m, 1H), 4.21-4.01 (m, 2H), 3.82-3.70 (m, 1H), 3.32-3.18 (m, 2H), 1.86-1.74 (m, 2H), 1.42-1.29 (m, 2H). ¹⁹F NMR (471 MHz, DMSO) δ −119.24 [−120.09], −121.21 [−122.04]. m/z (ES⁺), [M+H]⁺ 422.4. HPLC (A05) t_{R}=1.99 min.

### Preparation of Compound A-82.

### Step 1: N-[5-(5-fluoro-1H-benzimidazol-2-yl)-1-[(4-methoxyphenyl)-methyl]pyrazol-3-yl]-6-morpholino-pyridine-3-carboxamide

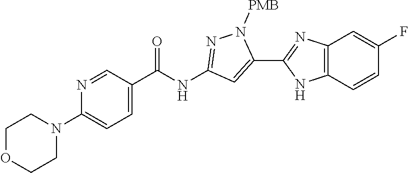

6-Chloro-N-[5-(5-fluoro-1H-benzimidazol-2-yl)-1-[(4-methoxyphenyl)methyl]pyrazol-3-yl]pyridine-3-carboxamide (L-2) (75 mg, 0.157 mmol), morpholine (20.3 μL, 0.236 mmol), and DIEA (82.2 μL, 0.472 mmol) were dissolved in DMSO (1 mL). The mixture was stirred at 100° C. for 18 h. Water (10 mL) was added. The mixture was filtered, and the solid was purified by silica gel chromatography eluting with hexanes and EtOAc (0-100%) to provide the title compound as a solid (57 mg, 69%). ¹H NMR (500 MHz, DMSO) δ 13.28 (s, 1H), 10.92 (s, 1H), 8.81 (d, J=2.3 Hz, 1H), 8.18 (dd, J=9.0, 2.5 Hz, 1H), 7.77-7.70 [7.57-7.55] (m, 1H), 7.56 (s, 1H), 7.57-7.55 [7.37-7.31] (m, 1H), 7.27-7.20 (m, 2H), 7.19-7.05 [7.19-7.05] (m, 1H), 6.89 (d, J=9.2 Hz, 1H), 6.87-6.84 (m, 2H), 6.01 (s, 2H), 3.73-3.68 (m, 4H), 3.69 (s, 3H), 3.62-3.57 (m, 4H). m/z (ES⁺), [M+H]⁺ 528.5. HPLC (A05) t_{R}=2.47 min.

### Step 2: N-[5-(5-fluoro-1H-benzimidazol-2-yl)-1H-pyrazol-3-yl]-6-morpholino-pyridine-3-carboxamide

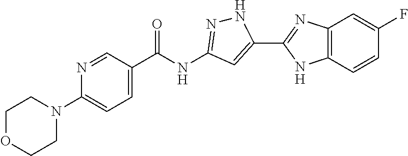 [SMILES: O=C(Nc1cc(-c2nc3cc(F)ccc3[nH]2)[nH]n1)c1ccc(N2CCOCC2)nc1]

TFA (1 mL) was added to N-[5-(5-fluoro-1H-benzimidazol-2-yl)-1-[(4-methoxyphenyl)methyl]pyrazol-3-yl]-6-morpholino-pyridine-3-carboxamide (47.9 mg, 0.0908 mmol), and the mixture was stirred at 23° C. for 1.5 h. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL) and dried under high vacuum to provide a 3:2 mixture of pyrazole tautomers of the title compound as a solid (22.1 mg, 60%). ¹H NMR (500 MHz, DMSO) δ 13.46 [13.16] (s, 1H), 13.16 [12.86] (s, 1H), 10.83 [10.99] (s, 1H), 8.82 (s, 1H), 8.26-8.05 (m, 1H), 7.76-7.50 (m, 1H), 7.42 [6.64] (s, 1H), 7.47-7.39 (m, 1H), 7.17-7.00 (m, 1H), 7.00-6.83 (m, 1H), 3.79-3.68 (m, 4H), 3.68-3.53 (m, 4H). ¹⁹F NMR (471 MHz, DMSO) δ −119.22 [−120.07], −121.20 [−122.03]. m/z (ES+), [M+H]⁺ 408.4. HPLC (A05) t_{R}=2.07 min.

### Preparation of Compound A-83.

### Step 1: N-[5-(7-fluoro-1H-benzimidazol-2-yl)-1-[(4-methoxyphenyl)methyl]-pyrazol-3-yl]-6-[4-(hydroxymethyl)-1-piperidyl]pyridine-3-carboxamide

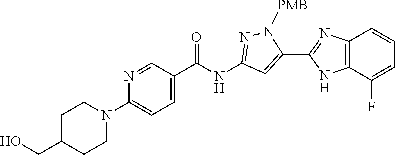

6-Chloro-N-[5-(7-fluoro-1H-benzimidazol-2-yl)-1-[(4-methoxyphenyl)methyl]pyrazol-3-yl]pyridine-3-carboxamide (L-3) (75 mg, 0.157 mmol), 4-piperidylmethanol (27.2 mg, 0.236 mmol), and DIEA (82.2 μL, 0.472 mmol) were dissolved in DMSO (1 mL). The mixture was stirred at 100° C. for 18 h. Water (10 mL) was added. The mixture was filtered, and the solid was purified by silica gel chromatography eluting with hexanes and EtOAc (0-100%) to provide the title compound as a solid (58.5 mg, 67%). ¹H NMR (500 MHz, DMSO) δ 13.43 (s, 1H), 10.86 (s, 1H), 8.78 (d, J=2.6 Hz, 1H), 8.12 (dd, J=9.1, 2.5 Hz, 1H), 7.57 (s, 1H), 7.39 (d, J=8.0 Hz, 1H), 7.30-7.20 (m, 3H), 7.10-7.03 (m, 1H), 6.89-6.84 (m, 3H), 6.01 (s, 2H), 4.52-4.38 (m, 3H), 3.70 (s, 3H), 3.31-3.24 (m, 2H), 2.96-2.78 (m, 2H), 1.77-1.60 (m, 3H), 1.17-1.03 (m, 2H). m/z (ES⁺), [M+H]⁺ 556.6. HPLC (A05) t_{R}=2.41 min.

### Step 2: N-[5-(7-fluoro-1H-benzimidazol-2-yl)-1H-pyrazol-3-yl]-6-[4-(hydroxymethyl)-1-piperidyl]pyridine-3-carboxamide.

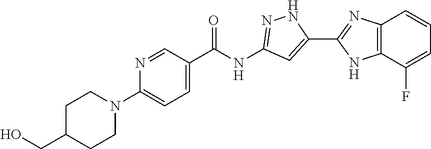 [SMILES: O=C(Nc1cc(-c2nc3cccc(F)c3[nH]2)[nH]n1)c1ccc(N2CCC(CO)CC2)nc1]

TFA (1 mL) was added to N-[5-(7-fluoro-1H-benzimidazol-2-yl)-1-[(4-methoxyphenyl)methyl]pyrazol-3-yl]-6-[4-(hydroxymethyl)-1-piperidyl]pyridine-3-carboxamide (55.3 mg, 0.0995 mmol), and the mixture was stirred at 23° C. for 30 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL) and dried under high vacuum to provide a 5:3 mixture of pyrazole tautomers of the title compound as a solid (29.4 mg, 68%). ¹H NMR (500 MHz, DMSO) δ 13.56 [13.22] (s, 1H), 13.34 [13.07] (s, 1H), 10.76 [10.91] (s, 1H), 8.78 (s, 1H), 8.26-7.89 (m, 1H), 7.59-7.41 (m, 1H), 7.41-7.30 (m, 1H), 7.22 [6.65] (s, 1H), 7.09-6.98 (m, 1H), 6.98-6.80 (m, 1H), 4.68-4.30 (m, 3H), 3.57-3.12 (m, 2H), 2.98-2.77 (m, 2H), 1.95-1.43 (m, 3H), 1.28-0.86 (m, 2H). ¹⁹F NMR (471 MHz, DMSO) δ −128.56 [−129.23]. m/z (ES+), [M+H]⁺ 436.4. HPLC (A05) t_{R}=2.03 min.

### Preparation of Compound A-84.

### Step 1: N-[5-(7-fluoro-1H-benzimidazol-2-yl)-1-[(4-methoxyphenyl)methyl]-pyrazol-3-yl]-6-(4-hydroxy-1-piperidyl)pyridine-3-carboxamide

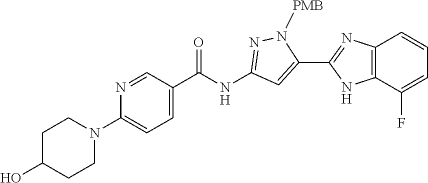

6-Chloro-N-[5-(7-fluoro-1H-benzimidazol-2-yl)-1-[(4-methoxyphenyl)methyl]pyrazol-3-yl]pyridine-3-carboxamide (L-3) (75 mg, 0.157 mmol), piperidin-4-ol (23.9 mg, 0.236 mmol), and DIEA (82.2 μL, 0.472 mmol) were dissolved in DMSO (1 mL). The mixture was stirred at 100° C. for 18 h. Water (10 mL) was added. The mixture was filtered, and the solid was purified by silica gel chromatography eluting with hexanes and EtOAc (0-100%) to provide the title compound as a solid (58.3 mg, 68%). ¹H NMR (500 MHz, DMSO) δ 13.44 (s, 1H), 10.87 (s, 1H), 8.78 (d, J=2.4 Hz, 1H), 8.13 (dd, J=9.1, 2.5 Hz, 1H), 7.58 (s, 1H), 7.39 (d, J=7.9 Hz, 1H), 7.30-7.20 (m, 3H), 7.09-7.03 (m, 1H), 6.90-6.85 (m, 3H), 6.01 (s, 2H), 4.74 (d, J=4.3 Hz, 1H), 4.16-4.05 (m, 2H), 3.79-3.72 (m, 1H), 3.70 (s, 3H), 3.30-3.20 (m, 2H), 1.89-1.72 (m, 2H), 1.43-1.30 (m, 2H). m/z (ES⁺), [M+H]⁺ 542.6. HPLC (A05) t_{R}=2.36 min.

### Step 2: N-[5-(7-fluoro-1H-benzimidazol-2-yl)-1H-pyrazol-3-yl]-6-[4-(hydroxymethyl)-1-piperidyl]pyridine-3-carboxamide

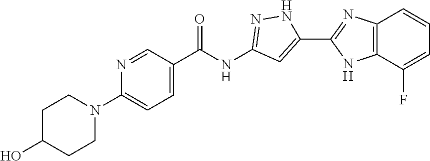 [SMILES: O=C(Nc1cc(-c2nc3cccc(F)c3[nH]2)[nH]n1)c1ccc(N2CCC(O)CC2)nc1]

TFA (1 mL) was added to N-[5-(7-fluoro-1H-benzimidazol-2-yl)-1-[(4-methoxyphenyl)methyl]pyrazol-3-yl]-6-[4-(hydroxymethyl)-1-piperidyl]pyridine-3-carboxamide (55.3 mg, 0.0995 mmol), and the mixture was stirred at 23° C. for 30 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL) and dried under high vacuum to provide a 5:3 mixture of pyrazole tautomers of the title compound as a solid (29.4 mg, 68%). ¹H NMR (500 MHz, DMSO) δ 13.56 [13.22] (s, 1H), 13.34 [13.07] (s, 1H), 10.76 [10.91] (s, 1H), 8.78 (s, 1H), 8.26-7.89 (m, 1H), 7.59-7.41 (m, 1H), 7.41-7.30 (m, 1H), 7.22 [6.65] (s, 1H), 7.09-6.98 (m, 1H), 6.98-6.80 (m, 1H), 4.68-4.30 (m, 3H), 3.57-3.12 (m, 2H), 2.98-2.77 (m, 2H), 1.95-1.43 (m, 3H), 1.28-0.86 (m, 2H). ¹⁹F NMR (471 MHz, DMSO) δ −128.56 [−129.23]. m/z (ES⁺), [M+H]⁺ 436.4. HPLC (A05) t_{R}=2.03 min.

### Preparation of Compound A-85.

### Step 1: N-[5-(7-fluoro-1H-benzimidazol-2-yl)-1-[(4-methoxyphenyl)methyl]-pyrazol-3-yl]-6-morpholino-pyridine-3-carboxamide

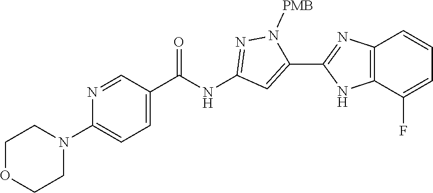

6-Chloro-N-[5-(7-fluoro-1H-benzimidazol-2-yl)-1-[(4-methoxyphenyl)methyl]pyrazol-3-yl]pyridine-3-carboxamide (L-3) (75 mg, 0.157 mmol), morpholine (20.3 μL, 0.236 mmol), and DIEA (82.2 μL, 0.472 mmol) were dissolved in DMSO (1 mL). The mixture was stirred at 100° C. for 18 h. Water (10 mL) was added. The mixture was filtered, and the solid was purified by silica gel chromatography eluting with hexanes and EtOAc (0-100%) to provide the title compound as a solid (52.1 mg, 63%). ¹H NMR (500 MHz, DMSO) δ 13.44 (s, 1H), 10.94 (s, 1H), 8.82 (d, J=2.4 Hz, 1H), 8.18 (dd, J=9.1, 2.5 Hz, 1H), 7.58 (s, 1H), 7.39 (d, J=7.9 Hz, 1H), 7.31-7.20 (m, 3H), 7.11-7.01 (m, 1H), 6.93-6.82 (m, 3H), 6.01 (s, 2H), 3.73-3.67 (m, 4H), 3.70 (s, 3H), 3.62-3.58 (m, 4H). m/z (ES⁺), [M+H]⁺ 528.5. HPLC (A05) t_{R}=2.48 min.

### Step 2: N-[5-(7-fluoro-1H-benzimidazol-2-yl)-1H-pyrazol-3-yl]-6-morpholino-pyridine-3-carboxamide

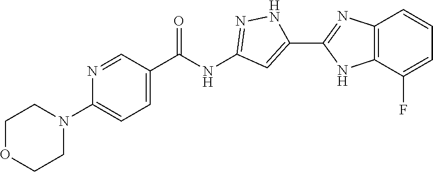 [SMILES: O=C(Nc1cc(-c2nc3cccc(F)c3[nH]2)[nH]n1)c1ccc(N2CCOCC2)nc1]

TFA (1 mL) was added to N-[5-(7-fluoro-1H-benzimidazol-2-yl)-1-[(4-methoxyphenyl)methyl]pyrazol-3-yl]-6-morpholino-pyridine-3-carboxamide (48.8 mg, 0.0925 mmol), and the mixture was stirred at 23° C. for 1.5 h. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL) and dried under high vacuum to provide a 5:3 mixture of pyrazole tautomers of the title compound as a solid (23.2 mg, 62%). ¹H NMR (500 MHz, DMSO) δ 13.60 [13.07] (s, 1H), 13.35 [13.07] (s, 1H), 10.84 [11.07] (s, 1H), 8.82 (s, 1H), 8.26-8.04 (m, 1H), 7.64-7.41 (m, 1H), 7.41-7.26 (m, 1H), 7.21 [6.71] (s, 1H), 7.12-6.98 (m, 1H), 6.98-6.84 (m, 1H), 3.79-3.67 (m, 4H), 3.67-3.54 (m, 4H). ¹⁹F NMR (471 MHz, DMSO) δ −128.56 [−129.21]. m/z (ES⁺), [M+H]⁺ 408.3. HPLC (A05) t_{R}=2.07 min.

### Preparation of Compound A-86.

### Step 1: N-[5-(5-chloro-1H-benzimidazol-2-yl)-1-[(4-methoxyphenyl)methyl]-pyrazol-3-yl]-6-[4-(hydroxymethyl)-1-piperidyl]pyridine-3-carboxamide

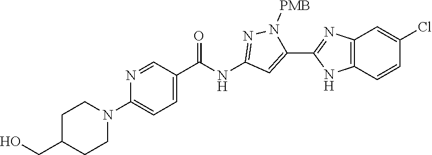

6-Chloro-N-[5-(5-chloro-1H-benzimidazol-2-yl)-1-[(4-methoxyphenyl)methyl]pyrazol-3-yl]pyridine-3-carboxamide (L-4) (74 mg, 0.150 mmol), 4-piperidylmethanol (0.0259 g, 0.225 mmol), and DIEA (0.0770 mL, 0.450 mmol) were dissolved in DMSO (1 mL). The mixture was stirred at 100° C. for 18 h. Water (25 mL) was added. The mixture was filtered, and the solid was purified by silica gel chromatography eluting with DCM and MeOH (0-10%) to provide the title compound as a solid (69 mg, 80%). ¹H NMR (400 MHz, DMSO) δ 13.31 (s, 1H), 10.85 (s, 1H), 8.76 (d, J=2.1 Hz, 1H), 8.10 (dd, J=9.1, 2.5 Hz, 1H), 7.74 (dd, J=19.3, 13.5 Hz, 1H), 7.59-7.51 (m, 2H), 7.33-7.18 (m, 3H), 6.85 (d, J=8.8 Hz, 3H), 5.99 (s, 2H), 4.47 (dd, J=15.2, 9.7 Hz, 3H), 3.68 (s, 3H), 3.27 (t, J=5.7 Hz, 2H), 2.88 (t, J=12.2 Hz, 2H), 1.70 (dd, J=21.9, 8.7 Hz, 3H), 1.10 (dd, J=21.4, 11.0 Hz, 2H). m/z (ES⁺), [M+H]⁺ 572.9. HPLC (A05) t_{R}=2.51 min.

### Step 2: N-[5-(5-chloro-1H-benzimidazol-2-yl)-1H-pyrazol-3-yl]-6-[4-(hydroxymethyl)-1-piperidyl]pyridine-3-carboxamide

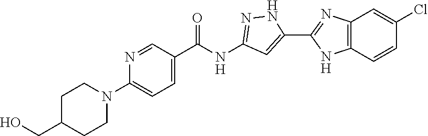 [SMILES: O=C(Nc1cc(-c2nc3cc(Cl)ccc3[nH]2)[nH]n1)c1ccc(N2CCC(CO)CC2)nc1]

TFA (2 mL) was added to N-[5-(5-chloro-1H-benzimidazol-2-yl)-1-[(4-methoxyphenyl)methyl]pyrazol-3-yl]-6-[4-(hydroxymethyl)-1-piperidyl]pyridine-3-carboxamide (69 mg, 0.121 mmol), and the mixture was stirred at 23° C. for 18 h. The mixture was concentrated. The residue was dissolved MeOH (10 mL) and stirred with LiOH (50 mg) and water (5 mL) for 1 h. The mixture was filtered, and the solid was dissolved in DMSO (0.800 mL). NEt₃ (0.800 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide a 7:4 mixture of tautomers of the title compound as a solid (46 mg, 84%). ¹H NMR (400 MHz, DMSO) δ 13.50 [12.93] (d, J=13.4 Hz, 1H), 13.29-13.10 (m, 1H), [10.88] 10.75 (s, 1H), 8.77 (s, 1H), 8.09 (t, J=10.0 Hz, 1H), 7.76-7.60 (m, 1H), 7.55 [6.62] (d, J=10.6 Hz, 1H), 7.43 (s, 1H), 7.22 (dd, J=36.0, 16.6 Hz, 1H), 6.89 (t, J=13.2 Hz, 1H), 4.47 (dd, J=12.3, 7.2 Hz, 3H), 3.30-3.24 (m, 2H), 2.90 (t, J=12.4 Hz, 2H), 1.70 (dd, J=29.1, 11.5 Hz, 3H), 1.19-1.04 (m, 2H). m/z (ES⁺), [M+H]⁺ 452.3. HPLC (A05) t_{R}=2.11 min.

### Preparation of Compound A-87.

### Step 1: N-[5-(5-chloro-1H-benzimidazol-2-yl)-1-[(4-methoxyphenyl)methyl]-pyrazol-3-yl]-6-(4-hydroxy-1-piperidyl)pyridine-3-carboxamide

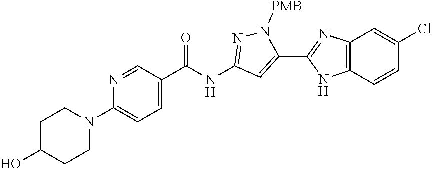

6-Chloro-N-[5-(5-chloro-1H-benzimidazol-2-yl)-1-[(4-methoxyphenyl)methyl]pyrazol-3-yl]pyridine-3-carboxamide (L-4) (74 mg, 0.150 mmol), piperidin-4-ol (0.0228 g, 0.225 mmol), and DIEA (0.0770 mL, 0.450 mmol) were dissolved in DMSO (1 mL). The mixture was stirred at 100° C. for 18 h. Water (25 mL) was added. The mixture was filtered, and the solid was purified by silica gel chromatography eluting with DCM and MeOH (0-10%) to provide the title compound as a solid (72 mg, 86%). ¹H NMR (400 MHz, DMSO) δ 13.32 (s, 1H), 10.86 (s, 1H), 8.77 (d, J=2.1 Hz, 1H), 8.11 (dd, J=9.1, 2.5 Hz, 1H), 7.82-7.66 (m, 1H), 7.62-7.50 (m, 2H), 7.27 (d, J=7.6 Hz, 1H), 7.22 (d, J=8.7 Hz, 2H), 6.86 (t, J=8.5 Hz, 3H), 5.99 (s, 2H), 4.73 (d, J=4.1 Hz, 1H), 4.10 (dt, J=8.9, 4.8 Hz, 2H), 3.79-3.71 (m, 1H), 3.68 (s, 3H), 3.23 (t, J=11.4 Hz, 2H), 1.79 (d, J=10.6 Hz, 2H), 1.35 (dd, J=18.8, 9.3 Hz, 2H). m/z (ES⁺), [M+H]⁺ 559.0. HPLC (A05) t_{R}=2.46 min.

### Step 2: N-[5-(5-chloro-1H-benzimidazol-2-yl)-1H-pyrazol-3-yl]-6-(4-hydroxy-1-piperidyl)pyridine-3-carboxamide

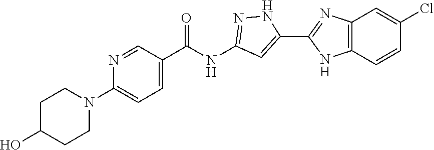 [SMILES: O=C(Nc1cc(-c2nc3cc(Cl)ccc3[nH]2)[nH]n1)c1ccc(N2CCC(O)CC2)nc1]

TFA (2 mL) was added to N-[5-(5-chloro-1H-benzimidazol-2-yl)-1-[(4-methoxyphenyl)methyl]pyrazol-3-yl]-6-(4-hydroxy-1-piperidyl)pyridine-3-carboxamide (72 mg, 0.129 mmol), and the mixture was stirred at 23° C. for 18 h. The mixture was concentrated. The residue was dissolved MeOH (10 mL) and stirred with LiOH (50 mg) and water (5 mL) for 1 h. The mixture was filtered, and the solid was dissolved in DMSO (0.800 mL). NEt₃ (0.800 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide a 7:4 mixture of tautomers of the title compound as a solid (34.5 mg, 61%). ¹H NMR (400 MHz, DMSO) δ 13.50 [12.93] (d, J=13.5 Hz, 1H), 13.28-13.13 (m, 1H), [10.90] 10.76 (s, 1H), 8.77 (s, 1H), 8.11 (d, J=8.8 Hz, 1H), 7.66 (dd, J=22.1, 15.2 Hz, 1H), 7.60-7.51 [6.62] (m, 1H), 7.43 (s, 1H), 7.30-7.16 (m, 1H), [6.95] 6.89 (d, J=9.0 Hz, 1H), 4.74 (d, J=3.5 Hz, 1H), 4.12 (d, J=13.7 Hz, 2H), 3.75 (dd, J=8.1, 4.0 Hz, 1H), 3.25 (dd, J=20.7, 9.8 Hz, 2H), 1.86-1.73 (m, 2H), 1.36 (td, J=13.0, 3.8 Hz, 2H). m/z (ES⁺), [M+H]⁺ 438.3. HPLC (A05) t_{R}=2.06 min.

### Preparation of Compound A-88.

### Step 1: N-[5-(5-chloro-1H-benzimidazol-2-yl)-1-[(4-methoxyphenyl)methyl]-pyrazol-3-yl]-6-morpholino-pyridine-3-carboxamide

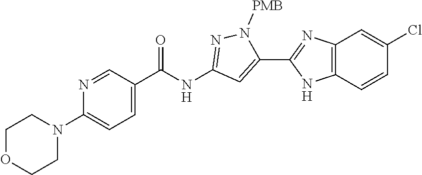

6-Chloro-N-[5-(5-chloro-1H-benzimidazol-2-yl)-1-[(4-methoxyphenyl)methyl]pyrazol-3-yl]pyridine-3-carboxamide (L-4) (74 mg, 0.150 mmol), morpholine (0.0195 mL, 0.225 mmol), and DIEA (0.0770 mL, 0.450 mmol) were dissolved in DMSO (1 mL). The mixture was stirred at 100° C. for 18 h. EtOAc (100 mL) was added. The organic phase was washed with water (3×50 mL) and brine (50 mL), dried (MgSO₄), filtered, and concentrated to provide the title compound as a solid (52 mg, 64%). ¹H NMR (400 MHz, DMSO) δ 13.34 [13.31] (s, 1H), 10.93 (s, 1H), 8.80 (d, J=2.3 Hz, 1H), 8.17 (dd, J=9.0, 2.5 Hz, 1H), 7.76 (dd, J=25.2, 5.0 Hz, 1H), 7.59-7.50 (m, 2H), 7.32-7.25 (m, 1H), 7.23 (d, J=8.5 Hz, 2H), 6.91-6.81 (m, 3H), 6.00 (s, 2H), 3.74-3.66 (m, 7H), 3.62-3.56 (m, 4H). m/z (ES⁺), [M+H]⁺ 544.5. HPLC (A05) t_{R}=2.58 min.

### Step 2: N-[5-(5-chloro-1H-benzimidazol-2-yl)-1H-pyrazol-3-yl]-6-morpholino-pyridine-3-carboxamide

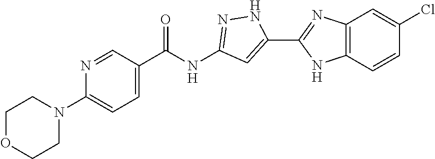 [SMILES: O=C(Nc1cc(-c2nc3cc(Cl)ccc3[nH]2)[nH]n1)c1ccc(N2CCOCC2)nc1]

TFA (2 mL) was added to N-[5-(5-chloro-1H-benzimidazol-2-yl)-1-[(4-methoxyphenyl)methyl]pyrazol-3-yl]-6-morpholino-pyridine-3-carboxamide (52 mg, 0.0956 mmol), and the mixture was stirred at 23° C. for 18 h. The mixture was concentrated. The residue was dissolved in DMSO (0.800 mL). NEt₃ (0.800 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide the title compound as a solid in a 2:1 ratio of tautomers (31.2 mg, 77%). ¹H NMR (400 MHz, DMSO) δ 13.52 [13.24] (d, J=13.9 Hz, 1H), 13.20 [12.93] (d, J=9.8 Hz, 1H), [10.96] 10.83 (s, 1H), 8.81 [8.80] (d, J=2.3 Hz, 1H), 8.18 [8.13] (dd, J=9.0, 2.6 Hz, 1H), 7.73-7.63 (m, 1H), 7.57 [6.63] (m, 1H), 7.48-7.42 (m, 1H), 7.31-7.16 (m, 1H), [6.97] 6.90 (d, J=9.3 Hz, 1H), 3.74-3.67 (m, 4H), 3.65-3.57 (m, 4H). m/z (ES⁺), [M+H]⁺ 424.3. HPLC (A05) t_{R}=2.16 min.

### Preparation of Compound A-89.

### Step 1: N-[5-(5-bromo-1H-benzimidazol-2-yl)-1-[(4-methoxyphenyl)methyl]pyrazol-3-yl]-6-[4-(hydroxymethyl)-1-piperidyl]pyridine-3-carboxamide

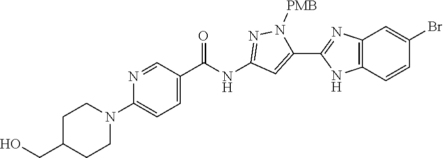

N-[5-(5-Bromo-1H-benzimidazol-2-yl)-1-[(4-methoxyphenyl)methyl]pyrazol-3-yl]-6-chloro-pyridine-3-carboxamide (L-5) (80.7 mg, 0.150 mmol), 4-piperidylmethanol (0.0259 g, 0.225 mmol), and DIEA (0.0770 mL, 0.450 mmol) were dissolved in DMSO (1 mL). The mixture was stirred at 100° C. for 18 h. Water (25 mL) was added. The mixture was filtered, and the solid was purified by silica gel chromatography eluting with DCM and MeOH (0-10%) to provide the title compound as a solid (84 mg, 91%). ¹H NMR (400 MHz, DMSO) δ 13.29 [13.25] (s, 1H), 10.80 (s, 1H), 8.72 (d, J=2.3 Hz, 1H), 8.06 (dd, J=9.1, 2.5 Hz, 1H), 7.90-7.86 [7.48-7.45] (m, 1H), 7.64 (d, J=12.3 Hz, 1H), 7.51 (s, 1H), 7.38-7.28 (m, 1H), 7.18 (d, J=8.6 Hz, 2H), 6.81 (d, J=8.7 Hz, 3H), 5.94 (s, 2H), 4.42 (dd, J=14.9, 9.6 Hz, 3H), 3.64 (s, 3H), 3.22 (t, J=5.7 Hz, 2H), 2.84 (t, J=12.6 Hz, 2H), 1.73-1.60 (m, 3H), 1.05 (dd, J=26.0, 12.7 Hz, 2H). m/z (ES⁺), [M+H]⁺ 617.4. HPLC (A05) t_{R}=2.65 min.

### Step 2: N-[5-(5-bromo-1H-benzimidazol-2-yl)-1H-pyrazol-3-yl]-6-[4-(hydroxymethyl)-1-piperidyl]pyridine-3-carboxamide

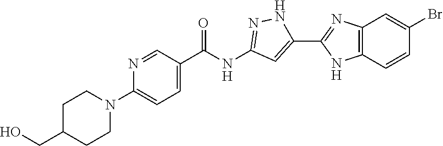 [SMILES: O=C(Nc1cc(-c2nc3cc(Br)ccc3[nH]2)[nH]n1)c1ccc(N2CCC(CO)CC2)nc1]

TFA (2 mL) was added to 3-chloro-N-[5-(5-methoxy-1H-benzimidazol-2-yl)-1-[(4-methoxyphenyl)methyl]pyrazol-3-yl]-4-(2-methoxyethoxy)benzamide (70 mg, 0.125 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The residue was purified by silica gel chromatography (12 g cartridge) eluting with DCM and MeOH (0-20%) to provide the title compound as a solid (48.5 mg, 72%). ¹H NMR (400 MHz, DMSO) δ 13.50 [12.93] (d, J=14.0 Hz, 1H), 13.28-13.13 (m, 1H), [10.89] 10.76 (s, 1H), 8.77 (s, 1H), 8.09 (t, J=11.3 Hz, 1H), 7.83 [7.51] (d, J=13.2 Hz, 1H), 7.71 [7.61] (s, 1H), 7.45-7.25 [6.62] (m, 2H), [6.94] 6.87 (d, J=8.6 Hz, 1H), 4.47 (dd, J=13.0, 7.9 Hz, 3H), 3.30-3.25 (m, 2H), 2.89 (t, J=11.4 Hz, 2H), 1.72 (t, J=15.2 Hz, 3H), 1.11 (qd, J=13.1, 4.2 Hz, 2H). m/z (ES⁺), [M+H]⁺ 497.7. HPLC (A05) t_{R}=2.14 min.

### Preparation of Compound A-90.

### Step 1: N-[5-(5-bromo-1H-benzimidazol-2-yl)-1-[(4-methoxyphenyl)methyl]-pyrazol-3-yl]-6-(4-hydroxy-1-piperidyl)pyridine-3-carboxamide

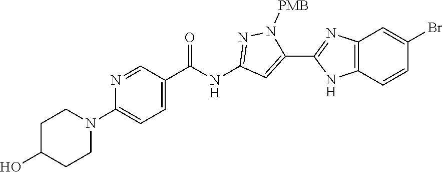

6-Chloro-N-[1-[(4-methoxyphenyl)methyl]-5-[5-(trifluoromethyl)-1H-benzimidazol-2-yl]pyrazol-3-yl]pyridine-3-carboxamide (L-5) (79 mg, 0.150 mmol), 4-piperidylmethanol (0.0259 g, 0.225 mmol), and DIEA (0.0770 mL, 0.450 mmol) were dissolved in DMSO (1 mL). The mixture was stirred at 100° C. for 18 h. Water (25 mL) was added. The mixture was filtered, and the solid was purified by silica gel chromatography eluting with hexanes and EtOAc (0-100%) to provide the title compound as a solid in a 1:1 ratio of rotamers (73 mg, 81%). ¹H NMR (400 MHz, DMSO) δ 13.33 [13.29] (s, 1H), 10.86 (s, 1H), 8.77 (d, J=2.9 Hz, 1H), 8.11 (dd, J=9.1, 2.5 Hz, 1H), 7.93 [7.70] (s, 1H), 7.69 [7.51] (d, J=7.9 Hz, 1H), 7.56 (s, 1H), 7.39 (ddd, J=17.1, 8.5, 1.6 Hz, 1H), 7.22 (dd, J=8.8, 2.5 Hz, 2H), 6.90-6.82 (m, 3H), 5.99 (s, 2H), 4.72 (s, 1H), 4.10 (dt, J=8.1, 3.4 Hz, 2H), 3.79-3.70 (m, 1H), 3.69 (s, 3H), 3.23 (ddd, J=13.1, 9.9, 3.2 Hz, 2H), 1.79 (dt, J=7.9, 5.3 Hz, 2H), 1.40-1.29 (m, 2H). m/z (ES⁺), [M+H]⁺ 602.9. HPLC (A05) t_{R}=2.48 min.

### Step 2: N-[5-(5-bromo-1H-benzimidazol-2-yl)-1H-pyrazol-3-yl]-6-(4-hydroxy-1-piperidyl)pyridine-3-carboxamide

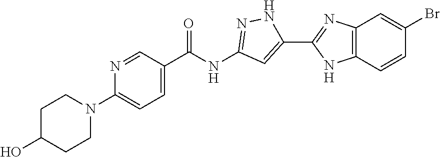 [SMILES: O=C(Nc1cc(-c2nc3cc(Br)ccc3[nH]2)[nH]n1)c1ccc(N2CCC(O)CC2)nc1]

TFA (2 mL) was added to N-[5-(5-bromo-1H-benzimidazol-2-yl)-1-[(4-methoxyphenyl)methyl]pyrazol-3-yl]-6-(4-hydroxy-1-piperidyl)pyridine-3-carboxamide (73 mg, 0.121 mmol), and the mixture was stirred at 23° C. for 1 h. The mixture was concentrated. The residue was dissolved in DMSO (0.400 mL). NEt₃ (0.800 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide the title compound as a solid in a 5:3 mixture of tautomers (48.5 mg, 83%). ¹H NMR (500 MHz, DMSO) δ 13.55-12.85 (m, 2H), [10.89] 10.76 (s, 1H), 8.79-8.73 (m, 1H), 8.10 (dd, J=20.4, 8.5 Hz, 1H), 7.83 [7.51] (d, J=18.2 Hz, 1H), [7.71] 7.60 (dd, J=20.1, 8.7 Hz, 1H), 7.43 [6.62] (s, 1H), 7.34 (ddd, J=32.6, 14.5, 8.3 Hz, 1H), [6.96] 6.89 (d, J=9.2 Hz, 1H), 4.73 (t, J=5.3 Hz, 1H), 4.11 (d, J=13.5 Hz, 2H), 3.76 (s, 1H), 3.25-3.20 (m, 2H), 1.80 (d, J=7.5 Hz, 2H), 1.36 (dd, J=19.2, 11.3 Hz, 2H). m/z (ES+), [M+H]⁺ 483.3. HPLC (A05) t_{R}=2.08 min.

### Preparation of Compound A-91.

### Step 1: N-[5-(5-bromo-1H-benzimidazol-2-yl)-1-[(4-methoxyphenyl)methyl]-pyrazol-3-yl]-6-morpholino-pyridine-3-carboxamide

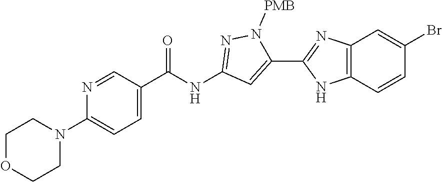

N-[5-(5-Bromo-1H-benzimidazol-2-yl)-1-[(4-methoxyphenyl)methyl]pyrazol-3-yl]-6-chloro-pyridine-3-carboxamide (L-5) (80.7 mg, 0.150 mmol), morpholine (0.0195 mL, 0.225 mmol), and DIEA (0.0770 mL, 0.450 mmol) were dissolved in DMSO (1 mL). The mixture was stirred at 100° C. for 18 h. EtOAc (100 mL) was added. The organic phase was washed with water (3×50 mL) and brine (50 mL), dried (MgSO₄), filtered, and concentrated to provide the title compound as a solid (87 mg, 99%). ¹H NMR (400 MHz, DMSO) δ 13.34 [13.30] (s, 1H), 10.93 (s, 1H), 8.80 (d, J=2.0 Hz, 1H), 8.17 (dd, J=9.0, 2.5 Hz, 1H), 7.93 [7.51] (s, 1H), 7.68 (d, J=12.4 Hz, 1H), 7.56 (s, 1H), 7.39 (dd, J=15.9, 8.4 Hz, 1H), 7.22 (d, J=7.1 Hz, 2H), 6.91-6.80 (m, 3H), 5.99 (s, 2H), 3.72-3.67 (m, 7H), 3.61-3.56 (m, 4H). m/z (ES⁺), [M+H]⁺ 589.3. HPLC (A05) t_{R}=2.74 min.

### Step 2: N-[5-(5-bromo-1H-benzimidazol-2-yl)-1H-pyrazol-3-yl]-6-morpholino-pyridine-3-carboxamide

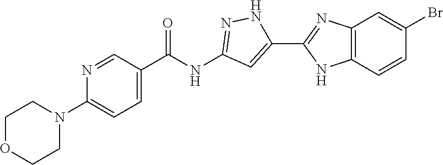 [SMILES: O=C(Nc1cc(-c2nc3cc(Br)ccc3[nH]2)[nH]n1)c1ccc(N2CCOCC2)nc1]

TFA (2 mL) was added to N-[5-(5-bromo-1H-benzimidazol-2-yl)-1-[(4-methoxyphenyl)methyl]pyrazol-3-yl]-6-morpholino-pyridine-3-carboxamide (87 mg, 0.148 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The residue was purified by silica gel chromatography (12 g cartridge) eluting with DCM and MeOH (0-20%) to provide the title compound as a solid (61.5 mg, 89%). ¹H NMR (400 MHz, DMSO) δ 13.53 [13.25] (d, J=16.3 Hz, 1H), 13.20 [12.94] (s, 1H), [10.96] 10.84 (s, 1H), 8.81 (s, 1H), 8.15 (dd, J=16.6, 9.4 Hz, 1H), 7.83 [7.71] (s, 1H), 7.60 [7.51] (dd, J=15.8, 8.0 Hz, 1H), 7.43 [6.63] (s, 1H) 7.40-7.26 (m, 1H), [6.96] 6.90 (d, J=8.5 Hz, 1H), 3.74-3.67 (m, 4H), 3.60 (d, J=4.6 Hz, 4H). m/z (ES⁺), [M+H]⁺ 468.2. HPLC (A05) t_{R}=2.19 min.

### Preparation of Compound A-98.

### Step 1: 6-[4-(hydroxymethyl)-1-piperidyl]-N-[1-[(4-methoxyphenyl)methyl]-5-(5-methyl-1H-benzimidazol-2-yl)pyrazol-3-yl]pyridine-3-carboxamide

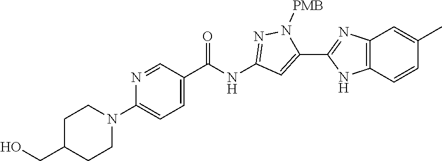

6-Chloro-N-[1-[(4-methoxyphenyl)methyl]-5-(5-methyl-1H-benzimidazol-2-yl)pyrazol-3-yl]pyridine-3-carboxamide (L-6) (70.9 mg, 0.150 mmol), 4-piperidylmethanol (0.0259 g, 0.225 mmol), and DIEA (0.0770 mL, 0.450 mmol) were dissolved in DMSO (1 mL). The mixture was stirred at 100° C. for 18 h. Water (25 mL) was added. The mixture was filtered, and the solid was purified by silica gel chromatography eluting with hexanes and EtOAc (0-100%) to provide the title compound as a solid in a 3:2 ratio of rotamers (67 mg, 81%). ¹H NMR (400 MHz, DMSO) δ 12.93 [12.89] (s, 1H), 10.77 (s, 1H), 8.72 (d, J=2.4 Hz, 1H), 8.06 (dd, J=9.1, 2.5 Hz, 1H), 7.53 [7.36] (d, J=7.6 Hz, 1H), 7.46 (s, 1H), 7.27 (s, 1H), 7.17 (d, J=8.8 Hz, 2H), [7.05] 7.00 (d, J=8.1 Hz, 1H), 6.83-6.78 (m, 3H), 5.96 (s, 2H), 4.47-4.34 (m, 3H), 3.64 (s, 3H), 3.22 (d, J=5.2 Hz, 2H), 2.84 (t, J=11.6 Hz, 2H), 2.40 [2.38] (s, 3H), 1.64 (m, 3H), 1.05 (dt, J=14.1, 11.9 Hz, 2H). m/z (ES⁺), [M+H]⁺ 552.1. HPLC (A05) t_{R}=2.43 min.

### Step 2: 6-[4-(hydroxymethyl)-1-piperidyl]-N-[5-(5-methyl-1H-benzimidazol-2-yl)-1H-pyrazol-3-yl]pyridine-3-carboxamide

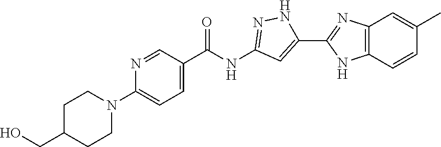 [SMILES: Cc1ccc2[nH]c(-c3cc(NC(=O)c4ccc(N5CCC(CO)CC5)nc4)n[nH]3)nc2c1]

TFA (2 mL) was added to 6-[4-(hydroxymethyl)-1-piperidyl]-N-[1-[(4-methoxyphenyl)-methyl]-5-(5-methyl-1H-benzimidazol-2-yl)pyrazol-3-yl]pyridine-3-carboxamide (67 mg, 0.121 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide the title compound as a solid in a 6:1 mixture of tautomers (37.7 mg, 66%). ¹H NMR (400 MHz, DMSO) δ 13.38 [13.07] (d, J=11.2 Hz, 1H), 12.85 [12.57] (d, J=16.4 Hz, 1H),[10.88] 10.72 (s, 1H), 8.77 (t, J=3.5 Hz, 1H), 8.14-8.04 (m, 1H), 7.53 [7.40] (d, J=8.2 Hz, 1H), 7.45 [7.31] (s, 1H), 7.37 [6.59] (s, 1H), 7.05 (dd, J=12.7, 8.5 Hz, 1H), [6.94] 6.87 (d, J=8.5 Hz, 1H), 4.47 (m, 3H), 3.30-3.26 (m, 2H), 2.90 (q, J=11.0 Hz, 2H), 2.44 [2.43] (s, 3H), 1.76-1.67 (m, 3H), 1.11 (dd, J=21.8, 10.8 Hz, 2H). m/z (ES⁺), [M+H]⁺ 432.4. HPLC (A05) t_{R}=2.01 min.

### Preparation of Compound A-99.

### Step 1: 6-(4-hydroxy-1-piperidyl)-N-[1-[(4-methoxyphenyl)methyl]-5-(5-methyl-1H-benzimidazol-2-yl)pyrazol-3-yl]pyridine-3-carboxamide

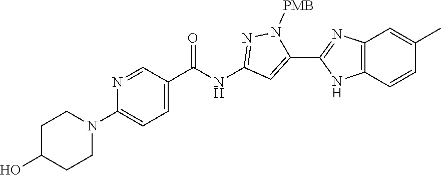

6-Chloro-N-[1-[(4-methoxyphenyl)methyl]-5-(5-methyl-1H-benzimidazol-2-yl)pyrazol-3-yl]-pyridine-3-carboxamide (L-6) (70.9 mg, 0.150 mmol), piperidin-4-ol (0.0228 g, 0.225 mmol), and DIEA (0.0770 mL, 0.450 mmol) were dissolved in DMSO (1 mL). The mixture was stirred at 100° C. for 18 h. Water (25 mL) was added. The mixture was filtered, and the solid was purified by silica gel chromatography eluting with hexanes and EtOAc (0-100%) to provide the title compound as a solid in a 4:3 ratio of rotamers (63 mg, 78%). ¹H NMR (400 MHz, DMSO) δ [12.98] 12.94 (s, 1H), 10.82 (s, 1H), 8.77 (d, J=2.3 Hz, 1H), 8.11 (dd, J=9.1, 2.6 Hz, 1H), 7.58 [7.41] (d, J=8.7 Hz, 1H), 7.51 (s, 1H), 7.32 (s, 1H), 7.22 (d, J=8.1 Hz, 2H), [7.10] 7.05 (d, J=8.3 Hz, 1H), 6.90-6.82 (m, 3H), 6.00 (s, 2H), 4.72 (s, 1H), 4.10 (dt, J=13.2, 4.4 Hz, 2H), 3.74 (qd, J=8.8, 3.9 Hz, 1H), 3.68 (s, 3H), 3.23 (ddd, J=13.0, 9.9, 3.0 Hz, 2H), 2.45 [2.43] (s, 3H), 1.79 (ddd, J=12.3, 8.3, 4.2 Hz, 2H), 1.40-1.30 (m, 2H). m/z (ES⁺), [M+H]⁺ 538.1. HPLC (A05) t_{R}=2.37 min.

### Step 2: 6-(4-hydroxy-1-piperidyl)-N-[5-(5-methyl-1H-benzimidazol-2-yl)-1H-pyrazol-3-yl]pyridine-3-carboxamide

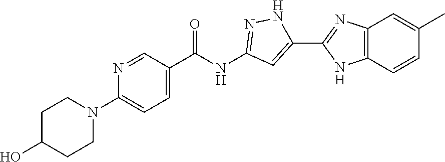 [SMILES: Cc1ccc2[nH]c(-c3cc(NC(=O)c4ccc(N5CCC(O)CC5)nc4)n[nH]3)nc2c1]

TFA (2 mL) was added to 6-(4-hydroxy-1-piperidyl)-N-[1-[(4-methoxyphenyl)methyl]-5-(5-methyl-1H-benzimidazol-2-yl)pyrazol-3-yl]pyridine-3-carboxamide (63 mg, 0.117 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The process was repeated a second time to provide the title compound as a solid in a 3:1 mixture of tautomers (33 mg, 68%). ¹H NMR (400 MHz, DMSO) δ 13.38 [13.12] (s, 1H), 12.84 [12.57] (s, 1H), [10.91] 10.73 (s, 1H), 8.77 (d, J=2.2 Hz, 1H), 8.11 (d, J=8.2 Hz, 1H), 7.56-7.13 [6.60] (m, 3H), 6.96 (dd, J=57.7, 8.0 Hz, 2H), 4.73 (s, 1H), 4.18-4.05 (m, 2H), 3.81-3.70 (m, 1H), 3.25 (dd, J=20.9, 10.6 Hz, 2H), 2.43 (s, 3H), 1.86-1.74 (m, 2H), 1.42-1.30 (m, 2H). m/z (ES⁺), [M+H]⁺ 418.3. HPLC (A05) t_{R}=1.97 min.

### Preparation of Compound A-100.

### Step 1: N-[5-(5-methyl-1H-benzimidazol-2-yl)-1H-pyrazol-3-yl]-6-morpholino-pyridine-3-carboxamide

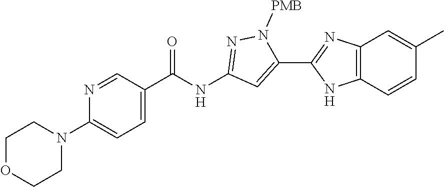

6-Chloro-N-[1-[(4-methoxyphenyl)methyl]-5-(5-methyl-1H-benzimidazol-2-yl)pyrazol-3-yl]pyridine-3-carboxamide (L-6) (70.9 mg, 0.150 mmol), morpholine (0.0194 mL, 0.225 mmol), and DIEA (0.0770 mL, 0.450 mmol) were dissolved in DMSO (1 mL). The mixture was stirred at 100° C. for 18 h. Water (25 mL) was added. The mixture was filtered, and the solid was purified by silica gel chromatography eluting with hexanes and EtOAc (0-100%) to provide the title compound as a solid in a 1:1 ratio of rotamers (61 mg, 78%). ¹H NMR (400 MHz, DMSO) δ [12.98] 12.94 (s, 1H), 10.89 (s, 1H), 8.80 (d, J=2.6 Hz, 1H), 8.17 (dd, J=9.1, 2.5 Hz, 1H), 7.58 [7.41] (d, J=8.5 Hz, 1H), 7.53-7.49 [7.33-7.30] (m, 2H), 7.22 (dd, J=8.4, 2.5 Hz, 2H), [7.10] 7.05 (d, J=8.8 Hz, 1H), 6.91-6.82 (m, 3H), 6.01 (s, 2H), 3.72-3.66 (m, 7H), 3.62-3.56 (m, 4H), 2.45 [2.43] (s, 3H). m/z (ES⁺), [M+H]⁺ 524.1. HPLC (A05) t_{R}=2.50 min.

### Step 2: N-[5-(5-methyl-1H-benzimidazol-2-yl)-1H-pyrazol-3-yl]-6-morpholino-pyridine-3-carboxamide

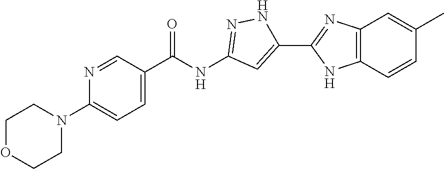 [SMILES: Cc1ccc2[nH]c(-c3cc(NC(=O)c4ccc(N5CCOCC5)nc4)n[nH]3)nc2c1]

TFA (2 mL) was added to N-[1-[(4-methoxyphenyl)methyl]-5-(5-methyl-1H-benzimidazol-2-yl)pyrazol-3-yl]-6-morpholino-pyridine-3-carboxamide (61 mg, 0.117 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide the title compound as a solid in a 3:1 mixture of tautomers (42.7 mg, 91%). ¹H NMR (400 MHz, DMSO) δ 13.40 [13.09] (d, J=11.3 Hz, 1H), 12.86 [12.57] (d, J=16.7 Hz, 1H), [10.94] 10.80 (s, 1H), 8.80 (dd, J=6.9, 2.2 Hz, 1H), 8.21-8.11 (m, 1H), 7.54 [7.41] (d, J=8.1 Hz, 1H), [7.46] 7.32 (s, 1H), 7.38 [7.24] (s, 1H), 7.06 (dd, J=11.8, 8.6 Hz, 1H), [6.96] 6.90 (d, J=9.6 Hz, 1H), 3.72-3.69 (m, 4H), 3.60 (dd, J=9.2, 4.4 Hz, 4H), [2.44] 2.43 (s, 3H). m/z (ES⁺), [M+H]⁺ 404.2. HPLC (A05) t_{R}=2.06 min.

### Preparation of Compound A-101.

### Step 1: 6-[4-(hydroxymethyl)-1-piperidyl]-N-[1-[(4-methoxyphenyl)methyl]-5-[5-(trifluoromethyl)-1H-benzimidazol-2-yl]pyrazol-3-yl]pyridine-3-carboxamide

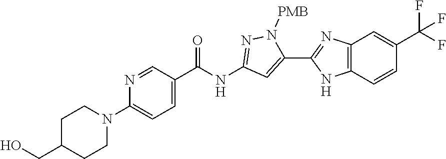

6-Chloro-N-[1-[(4-methoxyphenyl)methyl]-5-[5-(trifluoromethyl)-1H-benzimidazol-2-yl]pyrazol-3-yl]pyridine-3-carboxamide (L-7) (79 mg, 0.150 mmol), 4-piperidylmethanol (0.0259 g, 0.225 mmol), and DIEA (0.0770 mL, 0.450 mmol) were dissolved in DMSO (1 mL). The mixture was stirred at 100° C. for 18 h. Water (25 mL) was added. The mixture was filtered, and the solid was purified by silica gel chromatography eluting with hexanes and EtOAc (0-100%) to provide the title compound as a solid in a 5:3 ratio of rotamers (58 mg, 64%). ¹H NMR (400 MHz, DMSO) δ [13.54] 13.52 (s, 1H), 10.83 (s, 1H), 8.73 (d, J=2.6 Hz, 1H), 8.08 [7.88] (d, J=2.5 Hz, 1H), 8.05 (d, J=2.5 Hz, 1H), 7.79 [7.69] (s, 1H), 7.59-7.48 (m, 2H), 7.23-7.16 (m, 2H), 6.81 (d, J=8.8 Hz, 3H), 5.97 (s, 2H), 4.41 (dt, J=4.9, 4.4 Hz, 3H), 3.64 (s, 3H), 3.23 (t, J=5.0 Hz, 2H), 2.84 (t, J=11.5 Hz, 2H), 1.73-1.58 (m, 3H), 1.11-0.99 (m, 2H). m/z (ES⁺), [M+H]⁺ 606.1. HPLC (A05) t_{R}=2.55 min.

### Step 2: 6-[4-(hydroxymethyl)-1-piperidyl]-N-[5-[5-(trifluoromethyl)-1H-benzimidazol-2-yl]-1H-pyrazol-3-yl]pyridine-3-carboxamide

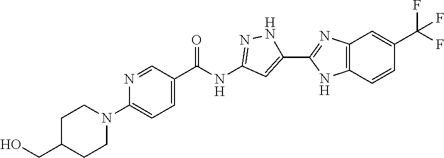 [SMILES: O=C(Nc1cc(-c2nc3cc(C(F)(F)F)ccc3[nH]2)[nH]n1)c1ccc(N2CCC(CO)CC2)nc1]

TFA (2 mL) was added to 6-[4-(hydroxymethyl)-1-piperidyl]-N-[1-[(4-methoxyphenyl)methyl]-5-[5-(trifluoromethyl)-1H-benzimidazol-2-yl]pyrazol-3-yl]pyridine-3-carboxamide (58 mg, 0.0958 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide the title compound as a solid in a 5:3 mixture of tautomers (31 mg, 67%). ¹H NMR (400 MHz, DMSO) δ [13.59] 13.48 (s, 1H), 13.23 (s, 1H), [10.91] 10.79 (s, 1H), 8.77 (s, 1H), 8.16-8.04 (m, 1H), 8.03-7.94 [7.59-7.43] (m, 2H), 7.90-7.67 (m, 1H), 6.99-6.58 (m, 2H), 4.48 (dd, J=11.8, 6.8 Hz, 3H), 3.29-3.26 (m, 2H), 2.90 (t, J=11.5 Hz, 2H), 1.78-1.64 (m, 3H), 1.17-1.06 (m, 2H). ¹⁹F NMR (376 MHz, DMSO) δ −58.95 (s). m/z (ES⁺), [M+H]⁺ 486.6. HPLC (A05) t_{R}=2.18 min.

### Preparation of Compound A-102.

### Step 1: N-[1-[(4-methoxyphenyl)methyl]-5-[5-(trifluoromethyl)-1H-benzimidazol-2-yl]pyrazol-3-yl]-6-morpholino-pyridine-3-carboxamide

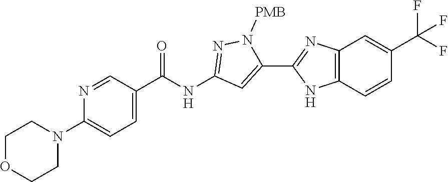

6-Chloro-N-[1-[(4-methoxyphenyl)methyl]-5-[5-(trifluoromethyl)-1H-benzimidazol-2-yl]pyrazol-3-yl]pyridine-3-carboxamide (L-7) (79 mg, 0.150 mmol), morpholine (0.0194 mL, 0.225 mmol), and DIEA (0.0770 mL, 0.450 mmol) were dissolved in DMSO (1 mL). The mixture was stirred at 100° C. for 18 h. EtOAc (100 mL) was added. The organic phase was washed with water (3×50 mL) and brine (50 mL), dried (Na₂SO₄), filtered, and concentrated. The residue was purified by silica gel chromatography eluting with hexanes and EtOAc (0-100%) to provide the title compound as a solid in a 4:3 ratio of rotamers (52 mg, 60%). ¹H NMR (400 MHz, DMSO) δ [13.59] 13.57 (s, 1H), 10.95 (s, 1H), 8.82-8.79 (m, 1H), 8.17 (dd, J=9.1, 2.5 Hz, 1H), 8.10 [7.84] (s, 1H), [7.93] 7.74 (d, J=8.7 Hz, 1H), 7.63-7.53 (m, 2H), 7.24 (dd, J=8.5, 5.9 Hz, 2H), 6.91-6.83 (m, 3H), 6.02 (s, 2H), 3.72-3.68 (m, 7H), 3.62-3.57 (m, 4H). m/z (ES+), [M+H]⁺ 578.1. HPLC (A05) t_{R}=2.62 min.

### Step 2: 6-morpholino-N-[5-[5-(trifluoromethyl)-1H-benzimidazol-2-yl]-1H-pyrazol-3-yl]pyridine-3-carboxamide

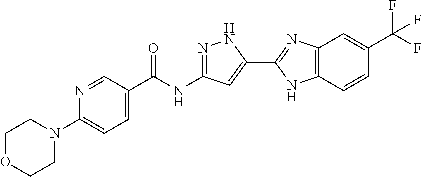 [SMILES: O=C(Nc1cc(-c2nc3cc(C(F)(F)F)ccc3[nH]2)[nH]n1)c1ccc(N2CCOCC2)nc1]

TFA (2 mL) was added to N-[1-[(4-methoxyphenyl)methyl]-5-[5-(trifluoromethyl)-1H-benzimidazol-2-yl]pyrazol-3-yl]-6-morpholino-pyridine-3-carboxamide (52 mg, 0.0900 mmol), and the mixture was stirred at 23° C. for 1 h. The mixture was concentrated, and the residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide the title compound as a solid (34.5 mg, 84%). ¹H NMR (400 MHz, DMSO) δ 13.80-13.06 (m, 2H), 10.88 (s, 1H), 8.81 (d, J=2.2 Hz, 1H), 8.17 (d, J=8.9 Hz, 1H), 8.08-7.40 [6.68] (m, 4H), 6.93(s, 1H), 3.72-3.69 (m, 4H), 3.62 (d, J=4.3 Hz, 4H). ¹⁹F NMR (376 MHz, DMSO) δ −58.85 (s). m/z (ES⁺), [M+H]⁺ 458.2. HPLC (A05) t_{R}=2.23 min.

### Preparation of Compound A-105.

### Step 1: N-[1-[(4-methoxyphenyl)methyl]-5-[5-(trifluoromethyl)-1H-benzimidazol-2-yl]pyrazol-3-yl]-6-morpholino-pyridine-3-carboxamide

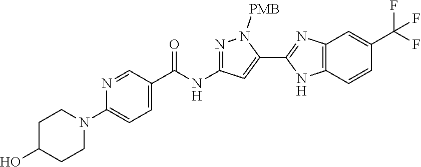

6-Chloro-N-[1-[(4-methoxyphenyl)methyl]-5-[5-(trifluoromethyl)-1H-benzimidazol-2-yl]pyrazol-3-yl]pyridine-3-carboxamide (L-7) (79 mg, 0.150 mmol), piperidin-4-ol (0.0228 g, 0.225 mmol), and DIEA (0.0770 mL, 0.450 mmol) were dissolved in DMSO (1 mL). The mixture was stirred at 100° C. for 18 h. EtOAc (100 mL) was added. The organic phase was washed with water (3×50 mL) and brine (50 mL), dried (Na₂SO₄), filtered, and concentrated. The residue was purified by silica gel chromatography eluting with hexanes and EtOAc (0-100%) to provide the title compound as a solid %) in a 5:3 ratio of rotamers (65 mg, 73. ¹H NMR (400 MHz, DMSO) δ [13.58] 13.56 (s, 1H), 10.88 (s, 1H), 8.77 (d, J=2.6 Hz, 1H), 8.13 [7.84] (d, J=2.5 Hz, 1H), 8.11 (d, J=2.5 Hz, 1H), [7.92] 7.74 (d, J=7.6 Hz, 1H), 7.63-7.53 (m, 2H), 7.24 (t, J=6.9 Hz, 2H), 6.90-6.83 (m, 3H), 6.02 (s, 2H), 4.73 (s, 1H), 4.10 (dt, J=13.8, 4.6 Hz, 2H), 3.79-3.70 (m, 1H), 3.69 (s, 3H), 3.23 (ddd, J=13.0, 9.7, 3.2 Hz, 2H), 1.83-1.73 (m, 2H), 1.40-1.30 (m, 2H). m/z (ES⁺), [M+H]⁺ 592.2. HPLC (A05) t_{R}=2.51 min.

### Step 2: 6-(4-hydroxy-1-piperidyl)-N-[5-[5-(trifluoromethyl)-1H-benzimidazol-2-yl]-1H-pyrazol-3-yl]pyridine-3-carboxamide

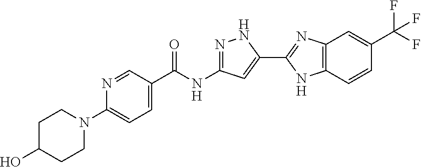 [SMILES: O=C(Nc1cc(-c2nc3cc(C(F)(F)F)ccc3[nH]2)[nH]n1)c1ccc(N2CCC(O)CC2)nc1]

TFA (2 mL) was added to 6-(4-hydroxy-1-piperidyl)-N-[1-[(4-methoxyphenyl)methyl]-5-[5-(trifluoromethyl)-1H-benzimidazol-2-yl]pyrazol-3-yl]pyridine-3-carboxamide (65 mg, 0.110 mmol), and the mixture was stirred at 23° C. for 1 h. The mixture was concentrated, and the residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide the title compound as a solid in a 6:1 mixture of tautomers (20.2 mg, 39%). ¹H NMR (400 MHz, DMSO) δ 13.55 [13.43] (s, 1H), 13.20 (s, 1H), [10.88] 10.75 (s, 1H), 8.73 (s, 1H), 8.06 (s, 1H), 7.98-7.38 (m, 3H), 7.01-6.59 (m, 2H), 4.69 (d, J=3.9 Hz, 1H), 4.14-4.01 (m, 2H), 3.73-3.68 (m, 1H), 3.25-3.13 (m, 2H), 1.81-1.69 (m, 2H), 1.31 (dt, J=12.9, 6.2 Hz, 2H). ¹⁹F NMR (376 MHz, DMSO) δ −58.77 (s). m/z (ES⁺), [M+H]⁺ 472.6. HPLC (A05) t_{R}=2.13 min.

### Preparation of Compound A-107.

### Step 1: N-[1-[(4-methoxyphenyl)methyl]-5-[5-(trifluoromethoxy)-1H-benzimidazol-2-yl]pyrazol-3-yl]-6-morpholino-pyridine-3-carboxamide

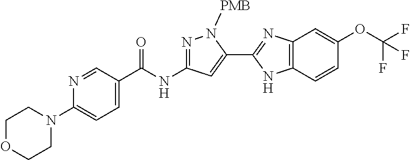

6-Chloro-N-[1-[(4-methoxyphenyl)methyl]-5-[5-(trifluoromethoxy)-1H-benzimidazol-2-yl]pyrazol-3-yl]pyridine-3-carboxamide (L-9) (81.4 mg, 0.150 mmol), morpholine (0.0194 mL, 0.225 mmol), and DIEA (0.0770 mL, 0.450 mmol) were dissolved in DMSO (1 mL). The mixture was stirred at 100° C. for 18 h. EtOAc (100 mL) was added. The organic phase was washed with water (3×50 mL) and brine (50 mL), dried (Na₂SO₄), filtered, and concentrated. The residue was purified by silica gel chromatography (12 g cartridge) eluting with DCM and MeOH (0-10%) to provide the title compound as a solid in a 2:1 ratio of tautomers (67 mg, 75%). ¹H NMR (400 MHz, DMSO) δ 13.43 (s, 1H), 10.93 (s, 1H), 8.81 (d, J=2.1 Hz, 1H), 8.17 (dd, J=9.0, 2.5 Hz, 1H), [7.81] 7.73 (s, 1H), 7.64 [7.50] (d, J=8.8 Hz, 1H), 7.57 (s, 1H), 7.29-7.18 (m, 3H), 6.90-6.83 (m, 3H), 6.01 (s, 2H), 3.72-3.67 (m, 7H), 3.61-3.56 (m, 4H). m/z (ES⁺), [M+H]⁺ 594.2. HPLC (A05) t_{R}=2.64 min.

### Step 2: 6-morpholino-N-[5-[5-(trifluoromethoxy)-1H-benzimidazol-2-yl]-1H-pyrazol-3-yl]pyridine-3-carboxamide

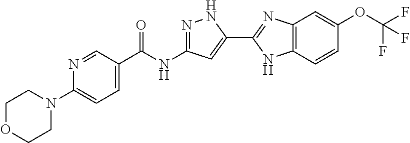 [SMILES: O=C(Nc1cc(-c2nc3cc(OC(F)(F)F)ccc3[nH]2)[nH]n1)c1ccc(N2CCOCC2)nc1]

TFA (2 mL) was added to N-[1-[(4-methoxyphenyl)methyl]-5-[5-(trifluoromethoxy)-1H-benzimidazol-2-yl]pyrazol-3-yl]-6-morpholino-pyridine-3-carboxamide (67 mg, 0.113 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide the title compound as a solid (39.4 mg, 74%). ¹H NMR (400 MHz, DMSO) δ 13.53 (s, 1H), 13.30 (s, 1H), 10.89 (s, 1H), 8.81 (d, J=2.4 Hz, 1H), 8.17 (d, J=9.2 Hz, 1H), 7.79-7.39 (m, 2H), 7.21 (d, J=7.8 Hz, 1H), [7.09-6.98] 6.85-6.72 (m, 1H), 6.93 (d, J=8.6 Hz, 1H), 3.72-3.69 (m, 4H), 3.63-3.57 (m, 4H). 19F NMR (376 MHz, DMSO) δ −57.01 (s). m/z (ES⁺), [M+H]⁺ 474.3. HPLC (A05) t_{R}=2.25 min.

### Preparation of Compound A-108.

### Step 1: 6-(4-hydroxy-1-piperidyl)-N-[1-[(4-methoxyphenyl)methyl]-5-[5-(trifluoromethoxy)-1H-benzimidazol-2-yl]pyrazol-3-yl]pyridine-3-carboxamide

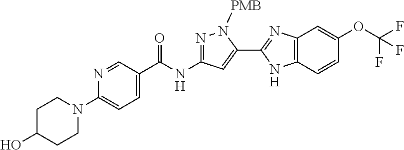

6-Chloro-N-[1-[(4-methoxyphenyl)methyl]-5-[5-(trifluoromethoxy)-1H-benzimidazol-2-yl]pyrazol-3-yl]pyridine-3-carboxamide (L-9) (81.4 mg, 0.150 mmol), piperidin-4-ol (0.0228 g, 0.225 mmol), and DIEA (0.0770 mL, 0.450 mmol) were dissolved in DMSO (1 mL). The mixture was stirred at 100° C. for 18 h. EtOAc (100 mL) was added. The organic phase was washed with water (3×50 mL) and brine (50 mL), dried (Na₂SO₄), filtered, and concentrated. The residue was purified by silica gel chromatography (12 g cartridge) eluting with DCM and MeOH (0-10%) to provide the title compound as a solid in a 3:2 ratio of rotamers (78 mg, 86%). ¹H NMR (400 MHz, DMSO) δ 13.43 (s, 1H), 10.87 (s, 1H), 8.77 (d, J=2.5 Hz, 1H), 8.11 (dd, J=9.1, 2.5 Hz, 1H), [7.82] 7.63 (d, J=8.3 Hz, 1H), 7.73 [7.51] (s, 1H), 7.57 (s, 1H), 7.30-7.19 (m, 3H), 6.91-6.83 (m, 3H), 6.00 (s, 2H), 4.73 (d, J=4.2 Hz, 1H), 4.10 (dt, J=12.2, 4.3 Hz, 2H), 3.75 (dq, J=12.6, 4.2 Hz, 1H), 3.69 (s, 3H), 3.23 (ddd, J=13.3, 10.3, 3.3 Hz, 2H), 1.79 (dd, J=12.7, 4.9 Hz, 2H), 1.40-1.29 (m, 2H). m/z (ES+), [M+H]⁺ 608.4. HPLC (A05) t_{R}=2.52 min.

### Step 2: 6-(4-hydroxy-1-piperidyl)-N-[5-[5-(trifluoromethoxy)-1H-benzimidazol-2-yl]-1H-pyrazol-3-yl]pyridine-3-carboxamide

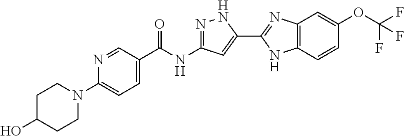 [SMILES: O=C(Nc1cc(-c2nc3cc(OC(F)(F)F)ccc3[nH]2)[nH]n1)c1ccc(N2CCC(O)CC2)nc1]

TFA (2 mL) was added to 6-(4-hydroxy-1-piperidyl)-N-[1-[(4-methoxyphenyl)methyl]-5-[5-(trifluoromethoxy)-1H-benzimidazol-2-yl]pyrazol-3-yl]pyridine-3-carboxamide (78 mg, 0.128 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide the title compound as a solid in a 2:1 mixture of tautomers (57.3 mg, 92%). ¹H NMR (400 MHz, DMSO) δ 13.48 [13.28] (s, 1H), 13.14 [13.00] (s, 1H), [10.86] 10.74 (s, 1H), 8.73 (s, 1H), 8.06 (s, 1H), 7.76-7.48 (m, 2H), 7.18 (s, 1H), 7.00-6.70 (m, 2H), 4.70 (s, 1H), 4.08 (dd, J=7.9, 5.4 Hz, 2H), 3.73-3.68 (m, 1H), 3.24-3.12 (m, 2H), 1.81-1.71 (m, 2H), 1.39-1.26 (m, 2H). ¹⁹F NMR (376 MHz, DMSO) δ −57.02 (s). m/z (ES⁺), [M+H]⁺ 488.9. HPLC (A05) t_{R}=2.15 min.

### Preparation of Compound A-109.

### Step 1: 6-[4-(hydroxymethyl)-1-piperidyl]-N-[5-(5-methoxy-1H-benzimidazol-2-yl)-1-[(4-methoxyphenyl)methyl]pyrazol-3-yl]pyridine-3-carboxamide

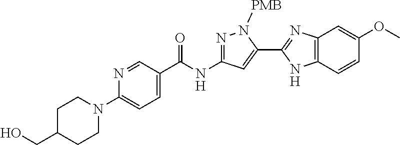

6-Chloro-N-[5-(5-methoxy-1H-benzimidazol-2-yl)-1-[(4-methoxyphenyl)methyl]pyrazol-3-yl]pyridine-3-carboxamide (L-8) (73.3 mg, 0.150 mmol), 4-piperidylmethanol (0.0259 g, 0.225 mmol), and DIEA (0.0770 mL, 0.450 mmol) were dissolved in DMSO (1 mL). The mixture was stirred at 100° C. for 18 h. EtOAc (100 mL) was added. The organic phase was washed with water (3×50 mL) and brine (50 mL), dried (Na₂SO₄), filtered, and concentrated. The residue was purified by silica gel chromatography (12 g cartridge) eluting with DCM and MeOH (0-10%) to provide the title compound as a solid in a 2:1 ratio of rotamers (59 mg, 69%). ¹H NMR (400 MHz, DMSO) δ [12.98] 12.95 (s, 1H), 10.81 (s, 1H), 8.76 (d, J=2.6 Hz, 1H), 8.10 (dd, J=9.1, 2.5 Hz, 1H), 7.59 [7.41] (d, J=9.0 Hz, 1H), 7.48 (s, 1H), 7.23 [6.98] (m, 3H), 6.87 (m, 4H), [6.01] 5.98 (s, 2H), 4.44 (m, 3H), 3.82 [3.80] (s, 3H), 3.69 (s, 3H), 3.27 (d, J=5.8 Hz, 2H), 2.92-2.82 (m, 2H), 1.71 (t, J=15.9 Hz, 3H), 1.10 (qd, J=12.9, 4.1 Hz, 2H). m/z (ES⁺), [M+H]⁺ 568.2. HPLC (A05) t_{R}=2.34 min.

### Step 2: 6-[4-(hydroxymethyl)-1-piperidyl]-N-[5-(5-methoxy-1H-benzimidazol-2-yl)-1H-pyrazol-3-yl]pyridine-3-carboxamide

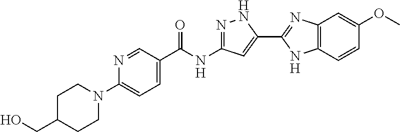 [SMILES: COc1ccc2[nH]c(-c3cc(NC(=O)c4ccc(N5CCC(CO)CC5)nc4)n[nH]3)nc2c1]

TFA (2 mL) was added to 6-[4-(hydroxymethyl)-1-piperidyl]-N-[5-(5-methoxy-1H-benzimidazol-2-yl)-1-[(4-methoxyphenyl)methyl]pyrazol-3-yl]pyridine-3-carboxamide (59 mg, 0.104 mmol), and the mixture was stirred at 23° C. for 30 min. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The wash process was repeated to provide the title compound as a solid in a 7:2 mixture of tautomers (25.5 mg, 55%). ¹H NMR (400 MHz, DMSO) δ 13.29 [13.00] (s, 1H), 12.79 [12.52] (s, 1H), [10.82] 10.68 (s, 1H), 8.73 (d, J=2.2 Hz, 1H), 8.06 (d, J=7.9 Hz, 1H), 7.49 [7.12] (d, J=7.9 Hz, 1H), 7.40-7.22 (m, 1H), 6.97-6.44 (m, 3H), 4.53-4.40 (m, 3H), 3.77 [3.76] (s, 3H), 3.26-3.21 (m, 2H), 2.86 (t, J=12.2 Hz, 2H), 1.75-1.57 (m, 3H), 1.07 (qd, J=12.5, 3.8 Hz, 2H). m/z (ES⁺), [M+H]⁺ 448.2. HPLC (A05) t_{R}=1.96 min.

### Preparation of Compound A-110.

### Step 1: 6-(4-hydroxy-1-piperidyl)-N-[5-(5-methoxy-1H-benzimidazol-2-yl)-1-[(4-methoxyphenyl)methyl]pyrazol-3-yl]pyridine-3-carboxamide

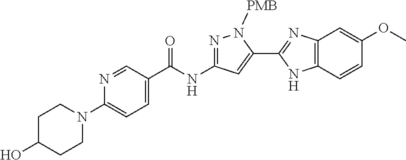

6-Chloro-N-[5-(5-methoxy-1H-benzimidazol-2-yl)-1-[(4-methoxyphenyl)methyl]pyrazol-3-yl]pyridine-3-carboxamide (L-8) (73.3 mg, 0.150 mmol), piperidin-4-ol (0.0228 g, 0.225 mmol), and DIEA (0.0770 mL, 0.450 mmol) were dissolved in DMSO (1 mL). The mixture was stirred at 100° C. for 18 h. EtOAc (100 mL) was added. The organic phase was washed with water (3×50 mL) and brine (50 mL), dried (Na₂SO₄), filtered, and concentrated. The residue was purified by silica gel chromatography (12 g cartridge) eluting with DCM and MeOH (0-10%) to provide the title compound as a solid in a 1:1 ratio of rotamers (67 mg, 81%). ¹H NMR (400 MHz, DMSO) δ [12.98] 12.95 (s, 1H), 10.81 (s, 1H), 8.76 (d, J=2.4 Hz, 1H), 8.11 (dd, J=9.0, 2.4 Hz, 1H), 7.59 [7.41] (d, J=9.2 Hz, 1H), 7.49 (s, 1H), 7.22 [6.98] (m, 3H), 6.87 (m, 4H), [6.01] 5.98 (s, 2H), 4.72 (s, 1H), 4.10 (dt, J=13.8, 4.4 Hz, 2H), 3.82 [3.80] (s, 3H), 3.73 (m, 1H), 3.69 (s, 3H), 3.23 (ddd, J=13.1, 9.9, 3.2 Hz, 2H), 1.79 (dt, J=7.9, 4.3 Hz, 2H), 1.40-1.29 (m, 2H). m/z (ES⁺), [M+H]⁺ 554.4. HPLC (A05) t_{R}=2.29 min.

### Step 2: 6-(4-hydroxy-1-piperidyl)-N-[5-(5-methoxy-1H-benzimidazol-2-yl)-1H-pyrazol-3-yl]pyridine-3-carboxamide

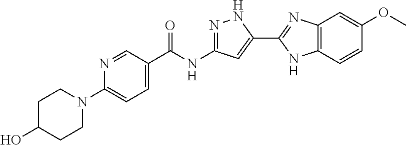 [SMILES: COc1ccc2[nH]c(-c3cc(NC(=O)c4ccc(N5CCC(O)CC5)nc4)n[nH]3)nc2c1]

TFA (2 mL) was added to 6-(4-hydroxy-1-piperidyl)-N-[5-(5-methoxy-1H-benzimidazol-2-yl)-1-[(4-methoxyphenyl)methyl]pyrazol-3-yl]pyridine-3-carboxamide (67 mg, 0.121 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The wash process was repeated to provide the title compound as a solid in a 3:1 mixture of tautomers (14.9 mg, 28%). ¹H NMR (400 MHz, DMSO) δ 13.33 [13.08] (s, 1H), 12.84 [12.56] (s, 1H), [10.91] 10.73 (s, 1H), 8.77 (d, J=2.3 Hz, 1H), 8.11 (d, J=8.8 Hz, 1H), 7.58-7.47 [7.15] (m, 1H), 7.43-7.28 (m, 1H), 7.04-6.66 (m, 3H), 4.73 (d, J=4.0 Hz, 1H), 4.11 (dt, J=12.3, 3.8 Hz, 2H), 3.79 (s, 3H), 3.77-3.71 (m, 1H), 3.24 (t, J=10.6 Hz, 2H), 1.85-1.73 (m, 2H), 1.42-1.30 (m, 2H). m/z (ES⁺), [M+H]⁺ 434.3. HPLC (A05) t_{R}=1.91 min.

### Preparation of Compound A-111.

### Step 1: 6-(4-hydroxy-1-piperidyl)-N-[5-(5-methoxy-1H-benzimidazol-2-yl)-1-[(4-methoxyphenyl)methyl]pyrazol-3-yl]pyridine-3-carboxamide

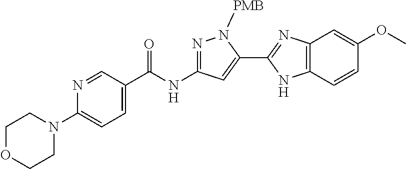

6-Chloro-N-[5-(5-methoxy-1H-benzimidazol-2-yl)-1-[(4-methoxyphenyl)methyl]pyrazol-3-yl]pyridine-3-carboxamide (L-8) (73.3 mg, 0.150 mmol), morpholine (0.0194 mL, 0.225 mmol), and DIEA (0.0770 mL, 0.450 mmol) were dissolved in DMSO (1 mL). The mixture was stirred at 100° C. for 18 h. EtOAc (100 mL) was added. The organic phase was washed with water (3×50 mL) and brine (50 mL), dried (Na₂SO₄), filtered, and concentrated. The residue was purified by silica gel chromatography (12 g cartridge) eluting with DCM and MeOH (0-10%) to provide the title compound as a solid in a 4:3 ratio of rotamers (53 mg, 66%). ¹H NMR (400 MHz, DMSO) δ [12.99] 12.95 (s, 1H), 10.88 (s, 1H), 8.80 (d, J=2.4 Hz, 1H), 8.17 (dd, J=8.8, 2.3 Hz, 1H), 7.59 [7.41] (d, J=8.8 Hz, 1H), 7.49 (d, J=2.6 Hz, 1H), 7.23 [6.98] (dd, J=11.2, 5.3 Hz, 3H), 6.92-6.82 (m, 4H), [6.01] 5.99 (s, 2H), 3.82 [3.80] (s, 3H), 3.71-3.66 (m, 7H), 3.61-3.56 (m, 4H). m/z (ES+), [M+H]⁺ 540.2. HPLC (A05) t_{R}=2.40 min.

### Step 2: N-[5-(5-methoxy-1H-benzimidazol-2-yl)-1H-pyrazol-3-yl]-6-morpholino-pyridine-3-carboxamide

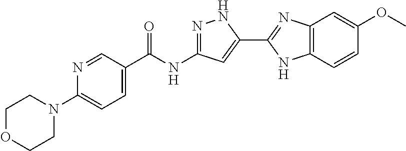 [SMILES: COc1ccc2[nH]c(-c3cc(NC(=O)c4ccc(N5CCOCC5)nc4)n[nH]3)nc2c1]

TFA (2 mL) was added to N-[5-(5-methoxy-1H-benzimidazol-2-yl)-1-[(4-methoxyphenyl)-methyl]pyrazol-3-yl]-6-morpholino-pyridine-3-carboxamide (53 mg, 0.0982 mmol), and the mixture was stirred at 23° C. for 30 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), and DCM (10 mL) and dried under high vacuum to provide a 7:2 mixture of pyrazole tautomers of the title compound as a solid (24.3 mg, 59%). ¹H NMR (400 MHz, DMSO) δ 13.35 [13.05] (s, 1H), 12.84 [12.59] (s, 1H), [10.91] 10.80 (s, 1H), 8.81 (d, J=2.1 Hz, 1H), 8.17 (d, J=7.2 Hz, 1H), 7.53 [7.16] (d, J=7.5 Hz, 1H), 7.38 (d, J=15.2 Hz, 1H), 7.01-6.78 (m, 3H), 3.81 [3.80] (s, 3H), 3.73-3.68 (m, 4H), 3.61 (d, J=4.2 Hz, 4H). m/z (ES⁺), [M+H]⁺ 420.2. HPLC (A05) t_{R}=2.00 min.

### Preparation of Compound A-114.

### Step 1: 6-[4-(hydroxymethyl)-1-piperidyl]-N-[1-[(4-methoxyphenyl)methyl]-5-[5-(trifluoromethoxy)-1H-benzimidazol-2-yl]pyrazol-3-yl]pyridine-3-carboxamide

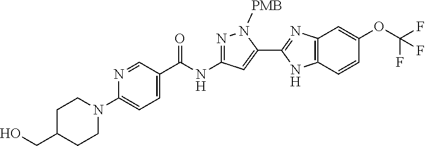

6-Chloro-N-[1-[(4-methoxyphenyl)methyl]-5-[5-(trifluoromethoxy)-1H-benzimidazol-2-yl]pyrazol-3-yl]pyridine-3-carboxamide (L-9) (81.4 mg, 0.150 mmol), 4-piperidylmethanol (0.0259 g, 0.225 mmol), and DIEA (0.0770 mL, 0.450 mmol) were dissolved in DMSO (1 mL). The mixture was stirred at 100° C. for 18 h. EtOAc (100 mL) was added. The organic phase was washed with water (3×50 mL) and brine (50 mL), dried (Na₂SO₄), filtered, and concentrated. The residue was purified by silica gel chromatography (12 g cartridge) eluting with DCM and MeOH (0-10%) to provide the title compound as a solid in a 5:3 ratio of rotamers (72 mg, 77%). ¹H NMR (400 MHz, DMSO) δ 13.38 (s, 1H), 10.81 (s, 1H), 8.72 (d, J=2.5 Hz, 1H), 8.06 (dd, J=9.1, 2.6 Hz, 1H), [7.77] 7.58 (d, J=8.8 Hz, 1H), 7.69 [7.45] (s, 1H), 7.53 (s, 1H), 7.26-7.14 (m, 3H), 6.81 (d, J=8.8 Hz, 3H), 5.96 (s, 2H), 4.42 (dd, J=14.2, 8.9 Hz, 3H), 3.64 (s, 3H), 3.23 (dd, J=9.8, 3.9 Hz, 2H), 2.84 (td, J=13.0, 2.3 Hz, 2H), 1.72-1.57 (m, 3H), 1.05 (ddd, J=24.5, 11.8, 3.4 Hz, 2H). m/z (ES⁺), [M+H]⁺ 622.2. HPLC (A05) t_{R}=2.57 min.

### Step 2: 6-[4-(hydroxymethyl)-1-piperidyl]-N-[5-[5-(trifluoromethoxy)-1H-benzimidazol-2-yl]-1H-pyrazol-3-yl]pyridine-3-carboxamide

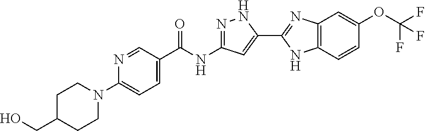 [SMILES: O=C(Nc1cc(-c2nc3cc(OC(F)(F)F)ccc3[nH]2)[nH]n1)c1ccc(N2CCC(CO)CC2)nc1]

TFA (2 mL) was added to 6-[4-(hydroxymethyl)-1-piperidyl]-N-[1-[(4-methoxyphenyl)methyl]-5-[5-(trifluoromethoxy)-1H-benzimidazol-2-yl]pyrazol-3-yl]pyridine-3-carboxamide (72 mg, 0.116 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The residue was purified by silica gel chromatography (4 g cartridge) eluting with DCM and MeOH (0-20%) to provide the title compound as a solid (28.3 mg, 49%). ¹H NMR (500 MHz, DMSO) δ 13.50 (s, 1H), 13.26 (s, 1H), 10.80 (s, 1H), 8.77 (d, J=2.4 Hz, 1H), 8.10 (d, J=10.6 Hz, 1H), 7.78-7.36 (m, 2H), 7.21 (s, 1H), 6.90 (s, 1H), 6.65 (s, 1H), 4.51-4.38 (m, 3H), 3.28 (d, J=6.1 Hz, 2H), 2.90 (t, J=13.2 Hz, 2H), 1.77-1.64 (m, 3H), 1.11 (dd, J=22.4, 10.1 Hz, 2H). ¹⁹F NMR (376 MHz, DMSO) δ −57.03 (s). m/z (ES⁺), [M+H]⁺ 502.7. HPLC (A05) t_{R}=2.20 min.

### Preparation of Compound A-131.

### Step 1: N-[5-(4-fluoro-1H-benzimidazol-2-yl)-1-[(4-methoxyphenyl)methyl]-pyrazol-3-yl]-6-(3-methoxyazetidin-1-yl)pyridine-3-carboxamide

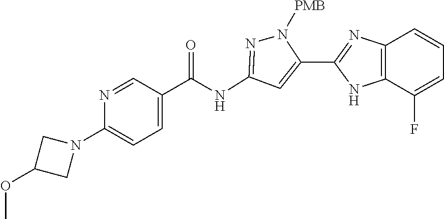

6-Chloro-N-[5-(5-fluoro-1H-benzimidazol-2-yl)-1-[(4-methoxyphenyl)methyl]pyrazol-3-yl]pyridine-3-carboxamide (L-3) (50 mg, 0.110 mmol), 3-methoxyazetidine hydrochloride (19.4 mg, 0.160 mmol), and DIEA (0.0548 mL, 0.315 mmol) were dissolved in DMSO (0.700 mL). The mixture was stirred at 100° C. for 18 h. Water (10 mL) was added. The mixture was filtered, and the solid was purified by silica gel chromatography (12 g cartridge) eluting with hexanes and EtOAc (0-100%) to provide the title compound as a solid (30 mg, 54%). ¹H NMR (400 MHz, DMSO) δ 13.43 (s, 1H), 10.90 (s, 1H), 8.76 (d, J=1.8 Hz, 1H), 8.13 (dd, J=8.8, 2.4 Hz, 1H), 7.56 (s, 1H), 7.41-7.35 (m, 1H), 7.25 (d, J=8.4 Hz, 3H), 7.10-7.02 (m, 1H), 6.86 (d, J=8.7 Hz, 2H), 6.43 (d, J=8.9 Hz, 1H), 6.00 (s, 2H), 4.39-4.33 (m, 1H), 4.26-4.21 (m, 2H), 3.85 (dd, J=10.3, 3.7 Hz, 2H), 3.69 (s, 3H), 3.26 (s, 3H). m/z (ES+), [M+H]⁺ 528.3. HPLC (A05) t_{R}=2.44 min.

### Step 2: N-[5-(7-fluoro-1H-benzimidazol-2-yl)-1H-pyrazol-3-yl]-6-(3-methoxy-azetidin-1-yl)pyridine-3-carboxamide

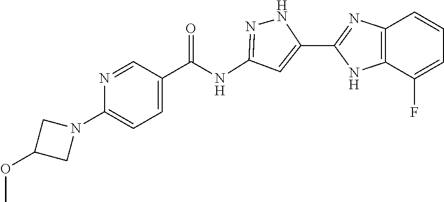 [SMILES: COC1CN(c2ccc(C(=O)Nc3cc(-c4nc5cccc(F)c5[nH]4)[nH]n3)cn2)C1]

TFA (1 mL) was added to N-[5-(4-fluoro-1H-benzimidazol-2-yl)-1-[(4-methoxyphenyl)-methyl]pyrazol-3-yl]-6-(3-methoxyazetidin-1-yl)pyridine-3-carboxamide (30 mg, 0.0569 mmol), and the mixture was stirred at 23° C. for 40 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with acetone (10 mL), DCM (10 mL), and acetone (10 mL) to provide the title compound as a solid (14 mg, 60%). ¹H NMR (500 MHz, DMSO) δ 13.90-12.47 (m, 2H), 10.92-10.71 (m, 1H), 8.76 (d, J=1.9 Hz, 1H), 8.13 (d, J=7.1 Hz, 1H), 7.51-7.27 (m, 2H), 7.26-7.16 (m, 1H), 7.07-6.96 (m, 1H), 6.48-6.42 (m, 1H), 4.36 (s, 1H), 4.27-4.21 (m, 2H), 3.86 (dd, J=9.5, 3.4 Hz, 2H), 3.27 (s, 3H). m/z (ES⁺), [M+H]⁺ 408.6. HPLC (A05) t_{R}=2.04 min.

### Preparation of Compound A-132.

### Step 1: N-[5-(5-fluoro-1H-benzimidazol-2-yl)-1-[(4-methoxyphenyl)methyl]-pyrazol-3-yl]-6-(3-methoxyazetidin-1-yl)pyridine-3-carboxamide

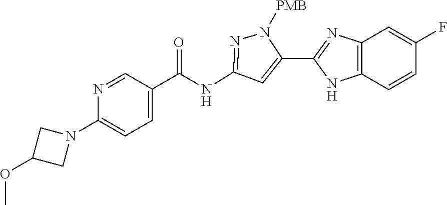

6-Chloro-N-[5-(5-fluoro-1H-benzimidazol-2-yl)-1-[(4-methoxyphenyl)methyl]pyrazol-3-yl]pyridine-3-carboxamide (L-2) (75 mg, 0.16 mmol), 3-methoxyazetidine hydrochloride (23.60 mg, 0.24 mmol), and DIEA (82.20 μL, 0.47 mmol) were dissolved in DMSO (1 mL). The mixture was stirred at 100° C. for 18 h. Water (10 mL) was added. The mixture was filtered, and the solid was purified by silica gel chromatography (12 g cartridge) eluting with hexanes and EtOAc (0-100%) to provide the title compound as an oil (38.1 mg, 46%). ¹H NMR (400 MHz, DMSO) δ 13.28 (s, 1H), 10.89 (s, 1H), 8.76 (s, 1H), 8.15-8.10 (m, 1H), 7.78-7.69 (m, 1H), 7.57-7.50 (m, 2H), 7.33 (m, 1H), 7.22 (d, J=8.1 Hz, 2H), 6.86 (d, J=8.7 Hz, 2H), 6.42 (d, J=8.9 Hz, 1H), 6.00 (s, 2H), 4.39-4.32 (m, 1H), 4.28-4.19 (m, 2H), 3.88-3.81 (m, 2H), 3.69 (s, 3H), 3.26 (s, 3H). m/z (ES⁺), [M+H]⁺ 529.3. HPLC (A05) t_{R}=2.44 min.

### Step 2: N-[5-(5-fluoro-1H-benzimidazol-2-yl)-1H-pyrazol-3-yl]-6-(3-methoxy-azetidin-1-yl)pyridine-3-carboxamide

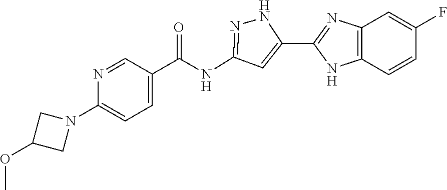 [SMILES: COC1CN(c2ccc(C(=O)Nc3cc(-c4nc5cc(F)ccc5[nH]4)[nH]n3)cn2)C1]

TFA (1 mL) was added to N-[5-(5-fluoro-1H-benzimidazol-2-yl)-1-[(4-methoxyphenyl)-methyl]pyrazol-3-yl]-6-(3-methoxyazetidin-1-yl)pyridine-3-carboxamide (57 mg, 0.108 mmol), and the mixture was stirred at 23° C. for 40 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with acetone (10 mL), DCM (10 mL), and acetone (10 mL) to provide a 2:1 mixture of pyrazole tautomers of the title compound as a solid (34 mg, 77%). ¹H NMR (500 MHz, DMSO) δ 8.76 (d, J=1.7 Hz, 1H), 8.13 (dd, J=8.7, 2.4 Hz, 1H), 7.58-7.50 (m, 1H), 7.36-7.29 (m, 1H), 7.18-7.07 (m, 1H), 7.07-6.97 (m, 1H), 6.45 (d, J=8.8 Hz, 1H), 4.36 (td, J=6.1, 3.1 Hz, 1H), 4.24 (ddd, J=9.2, 6.1, 0.8 Hz, 2H), 3.86 (dd, J=10.3, 4.0 Hz, 2H), 3.27 (s, 3H). m/z (ES⁺), [M+H]⁺ 408.6. HPLC (A05) t_{R}=2.04 min.

### Preparation of Compound A-142.

### Step 1: N-[5-(1H-benzimidazol-2-yl)-1-[(4-methoxyphenyl)methyl]pyrazol-3-yl]-6-[4-(2-methoxyethyl)piperazin-1-yl]pyridine-3-carboxamide

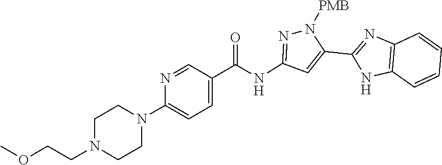

N-[5-(1H-Benzimidazol-2-yl)-1-[(4-methoxyphenyl)methyl]pyrazol-3-yl]-6-chloro-pyridine-3-carboxamide (L-1) (60 mg, 0.131 mmol), 1-(2-methoxyethyl)piperazine (0.0226 g, 0.157 mmol), and DIEA (0.0448 mL, 0.261 mmol) were dissolved in DMSO (1.30 mL). The mixture was heated at 100° C. for 18 h. EtOAc (100 mL) was added. The organic phase was washed with water (3×50 mL) and brine (50 mL), dried (Na₂SO₄), filtered, and concentrated. The residue was purified by silica gel chromatography (12 g cartridge) eluting with DCM and MeOH (0-10%) to provide the title compound as a solid (45 mg, 61%). ¹H NMR (400 MHz, DMSO) δ 13.12 (s, 1H), 10.87 (s, 1H), 8.78 (d, J=2.5 Hz, 1H), 8.14 (dd, J=9.1, 2.6 Hz, 1H), 7.72 (d, J=7.7 Hz, 1H), 7.54 (dd, J=6.5, 2.2 Hz, 2H), 7.31-7.20 (m, 4H), 6.89-6.82 (m, 3H), 6.02 (s, 2H), 3.68 (s, 3H), 3.61 (t, J=5.4 Hz, 4H), 3.47 (t, J=5.7 Hz, 2H), 3.25 (s, 3H), 2.55-2.51 (m, 6H). m/z (ES⁺), [M+H]⁺ 567.3. HPLC (A05) t_{R}=2.12 min.

### Step 2: N-[5-(1H-benzimidazol-2-yl)-1H-pyrazol-3-yl]-6-[4-(2-methoxyethyl)-piperazin-1-yl]pyridine-3-carboxamide

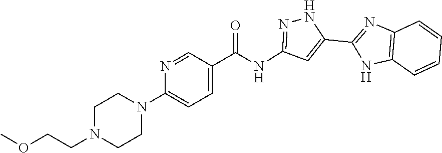 [SMILES: COCCN1CCN(c2ccc(C(=O)Nc3cc(-c4nc5ccccc5[nH]4)[nH]n3)cn2)CC1]

TFA (1 mL) was added to N-[5-(1H-benzimidazol-2-yl)-1-[(4-methoxyphenyl)methyl]pyrazol-3-yl]-6-[4-(2-methoxyethyl)piperazin-1-yl]pyridine-3-carboxamide (45 mg, 0.0794 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 10 min. Water (10 mL) was added. The mixture was filtered, and the solid was washed with water (10 mL), acetone (10 mL), DCM (10 mL), and acetone (10 mL) to provide the title compound as a solid in a 3:1 mixture of tautomers (29.5 mg, 83%). ¹H NMR (400 MHz, DMSO) δ 13.42 [13.14] (s, 1H), 12.98 [12.71] (s, 1H), [10.89] 10.74 (s, 1H), 8.75 (d, J=2.2 Hz, 1H), 8.10 (d, J=8.4 Hz, 1H), 7.69-7.55 (m, 1H), 7.49 (t, J=19.8 Hz, 1H), 7.37 (s, 1H), 7.26-7.10 (m, 2H), 6.85 (d, J=8.2 Hz, 1H), 3.66-3.55 (m, 4H), 3.44 (t, J=5.8 Hz, 2H), 3.21 (s, 3H), 2.49 (t, J=4.2 Hz, 6H). m/z (ES⁺), [M+H]⁺ 447.1. HPLC (A05) t_{R}=1.77 min.

### Preparation of Compound A-143.

### Step 1: N-[5-(1H-benzimidazol-2-yl)-1-[(4-methoxyphenyl)methyl]pyrazol-3-yl]-6-[4-(2-hydroxyethyl)piperazin-1-yl]pyridine-3-carboxamide

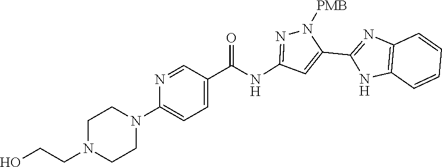

DIEA (0.0448 mL, 0.261 mmol) was added to a solution of N-[5-(1H-benzimidazol-2-yl)-1-[(4-methoxyphenyl)methyl]pyrazol-3-yl]-6-chloro-pyridine-3-carboxamide (L-1) (60 mg, 0.131 mmol) and 2-piperazin-1-ylethanol (0.0204 g, 0.157 mmol) in DMSO (1.30 mL). The mixture was stirred at 100° C. for 18 h. EtOAc (100 mL) was added. The organic phase was washed with water (3×50 mL) and brine (50 mL), dried (Na₂SO₄), filtered, and concentrated. The residue was purified by silica gel chromatography (12 g cartridge) eluting with DCM and MeOH (0-20%) to provide the title compound as a solid (43 mg, 60%). ¹H NMR (400 MHz, DMSO) δ 13.08 (s, 1H), 10.83 (s, 1H), 8.74 (d, J=2.4 Hz, 1H), 8.10 (dd, J=9.1, 2.5 Hz, 1H), 7.68 (d, J=7.8 Hz, 1H), 7.52-7.47 (m, 2H), 7.27-7.15 (m, 4H), 6.82 (dq, J=5.0, 2.8 Hz, 3H), 5.98 (s, 2H), 4.40 (t, J=5.3 Hz, 1H), 3.64 (s, 3H), 3.60-3.55 (m, 4H), 3.50 (q, J=6.1 Hz, 2H), 2.48 (d, J=1.9 Hz, 4H), 2.39 (t, J=6.1 Hz, 2H). m/z (ES⁺), [M+H]⁺ 553.3. HPLC (A05) t_{R}=2.04 min.

### Step 2: N-[5-(1H-benzimidazol-2-yl)-1H-pyrazol-3-yl]-6-[4-(2-hydroxyethyl)-piperazin-1-yl]pyridine-3-carboxamide

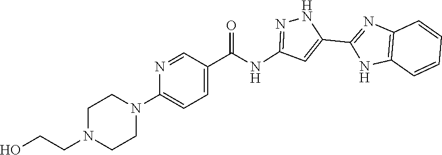 [SMILES: O=C(Nc1cc(-c2nc3ccccc3[nH]2)[nH]n1)c1ccc(N2CCN(CCO)CC2)nc1]

TFA (1 mL) was added to N-[5-(1H-benzimidazol-2-yl)-1-[(4-methoxyphenyl)methyl]pyrazol-3-yl]-6-[4-(2-hydroxyethyl)piperazin-1-yl]pyridine-3-carboxamide (43 mg, 0.0778 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide the title compound as a solid in a 5:2 mixture of tautomers (11.2 mg, 33%). ¹H NMR (400 MHz, DMSO) δ 13.44 [13.12] (s, 1H), 13.01 [12.73] (s, 1H), [10.93] 10.78 (s, 1H), 8.79 (s, 1H), 8.15-8.07 (m, 1H), 7.67 [7.62] (d, 8.5 Hz, 1H), 7.53 [7.46] (d, J=7.5 Hz, 1H), 7.41 [6.63] (s, 1H), 7.27-7.13 (m, 2H), [6.95] 6.88 (d, J=8.1 Hz, 1H), 4.44 (t, J=5.3 Hz, 1H), 3.67-3.59 (m, 4H), 3.54 (dd, J=11.6, 6.1 Hz, 2H), 2.53-2.51 (m, 4H), 2.44 (t, J=6.2 Hz, 2H). m/z (ES⁺), [M+H]⁺ 433.3. HPLC (A05) t_{R}=1.72 min.

### Preparation of Compound A-156 (2-[4-[5-[[5-(1H-benzimidazol-2-yl)-1H-pyrazol-3-yl]carbamoyl]-2-pyridyl]piperazin-1-yl]acetic acid)

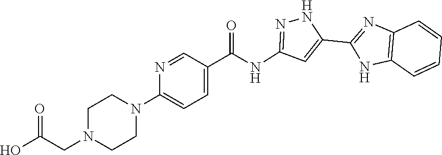 [SMILES: O=C(O)CN1CCN(c2ccc(C(=O)Nc3cc(-c4nc5ccccc5[nH]4)[nH]n3)cn2)CC1]

Ethyl 2-piperazin-1-ylacetate (0.0563 g, 0.327 mmol) was added to a solution of N-[5-(1H-benzimidazol-2-yl)-1-[(4-methoxyphenyl)methyl]pyrazol-3-yl]-6-chloro-pyridine-3-carboxamide (L-1) (100 mg, 0.218 mmol) and DIEA (0.112 mL, 0.654 mmol) in EtOH (2 mL) The mixture was stirred at 100° C. for 96 h. EtOAc (100 mL) was added, and the organic phase was washed with water (3×30 mL) and brine (50 mL), dried (Na₂SO₄), filtered, and concentrated. The residue was purified by silica gel chromatography (12 g cartridge) eluting with DCM and MeOH (0-40%). TFA (1 mL) was added to the purified residue, and the mixture was stirred for 1 h at 23° C. The mixture was concentrated. The residue was purified by preparative HPLC (BEH C18 30×150) eluting with water (10 mM (NH₄)(HCO₃)) and MeCN (17-27%) to provide the title compound as a solid (9.1 mg, 9%). ¹H NMR (400 MHz, DMSO) δ 13.44 (s, 1H), 12.98 (s, 1H), 12.69 (s, 1H), 10.75 (s, 1H), 8.79 (s, 1H), 8.12 (dd, J=8.9, 2.4 Hz, 1H), 7.61 (s, 1H), 7.47 (s, 1H), 7.35 (s, 1H), 7.17 (s, 2H), 6.88 (d, J=10.6 Hz, 1H), 3.67-3.61 (m, 4H), 3.14 (s, 2H), 2.71-2.57 (m, 4H). m/z (ES⁺), [M+H]⁺ 447.3. HPLC (A05) t_{R}=1.74 min.

### Preparation of Compound A-159.

### Step 1: N-[5-(1H-benzimidazol-2-yl)-1-[(4-methoxyphenyl)methyl]pyrazol-3-yl]-6-(4-methoxy-1-piperidyl)pyridine-3-carboxamide

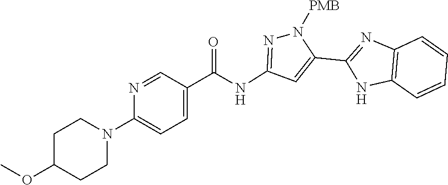

4-Methoxypiperidine (0.0166 g, 0.144 mmol) was added to a solution of N-[5-(1H-benzimidazol-2-yl)-1-[(4-methoxyphenyl)methyl]pyrazol-3-yl]-6-chloro-pyridine-3-carboxamide (L-1) (60 mg, 0.131 mmol) and DIEA (0.0448 mL, 0.261 mmol) in DMSO (1 mL) The mixture was stirred at 100° C. for 18 h. Water (20 mL) was added, and the mixture was filtered. The solid was purified by silica gel chromatography (12 g cartridge) eluting with DCM and MeOH (0-5%) to provide the title compound as a solid (19.9 mg, 28%). ¹H NMR (400 MHz, DMSO) δ 13.11 (s, 1H), 10.84 (s, 1H), 8.77 (d, J=2.5 Hz, 1H), 8.12 (dd, J=9.1, 2.5 Hz, 1H), 7.71 (d, J=6.1 Hz, 1H), 7.57-7.50 (m, 2H), 7.30-7.20 (m, 4H), 6.91-6.83 (m, 3H), 6.02 (s, 2H), 4.07-3.99 (m, 2H), 3.68 (s, 3H), 3.49-3.42 (m, 1H), 3.35-3.29 (m, 2H), 3.28 (s, 3H), 1.89 (ddd, J=9.5, 6.9, 3.8 Hz, 2H), 1.47-1.37 (m, 2H). m/z (ES⁺), [M+H]⁺ 538.8. HPLC (A05) t_{R}=2.51 min.

### Step 2: N-[5-(1H-benzimidazol-2-yl)-1H-pyrazol-3-yl]-6-(4-methoxy-1-piperidyl)-pyridine-3-carboxamide

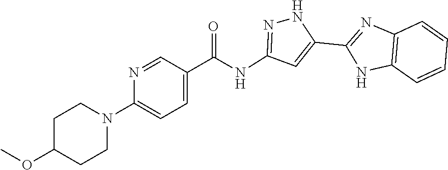 [SMILES: COC1CCN(c2ccc(C(=O)Nc3cc(-c4nc5ccccc5[nH]4)[nH]n3)cn2)CC1]

TFA (1 mL) was added to N-[5-(1H-benzimidazol-2-yl)-1-[(4-methoxyphenyl)methyl]pyrazol-3-yl]-6-(4-methoxy-1-piperidyl)pyridine-3-carboxamide (19.9 mg, 0.0370 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The residue was purified by preparative HPLC (BEH C18 30×150) eluting with water (10 mM (NH₄)(HCO₃)) and MeCN (36-46%) to provide the title compound as a solid (11.4 mg, 74%). ¹H NMR (400 MHz, DMSO) δ 8.78 (d, J=2.4 Hz, 1H), 8.13 (dd, J=9.0, 2.5 Hz, 1H), 7.55 (dd, J=5.8, 3.2 Hz, 2H), 7.16 (dd, J=5.9, 3.1 Hz, 2H), 7.08 (s, 1H), 6.91 (d, J=9.1 Hz, 1H), 4.04 (dt, J=9.7, 4.2 Hz, 2H), 3.50-3.42 (m, 1H), 3.35-3.27 (m, 5H), 1.94-1.84 (m, 2H), 1.49-1.38 (m, 2H). m/z (ES⁺), [M+H]⁺ 418.1. HPLC (A05) t_{R}=2.10 min.

### Preparation of Compound A-169.

### Step 1: N-[5-(1H-benzimidazol-2-yl)-1-[(4-methoxyphenyl)methyl]pyrazol-3-yl]-6-[4-[methyl(methylsulfonyl)amino]-1-piperidyl]pyridine-3-carboxamide

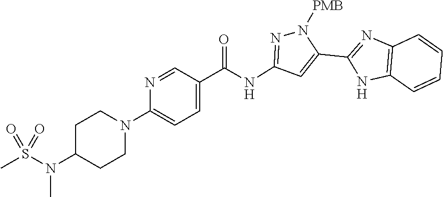

N-[5-(1H-Benzimidazol-2-yl)-1-[(4-methoxyphenyl)methyl]pyrazol-3-yl]-6-chloro-pyridine-3-carboxamide (L-1) (50 mg, 0.109 mmol) was dissolved in DMSO (1 mL). DIEA (38.0 μL, 0.218 mmol), and N-methyl-N-(4-piperidyl)methanesulfonamide (31.4 mg, 0.163 mmol) were added. The mixture was heated at 100° C. for 18 h. Water (15 mL) was added, and the mixture was stirred at 23° C. for 5 min. The mixture was filtered, and the solid was purified by silica gel chromatography (12 g cartridge) eluting with DCM and MeOH (0-10%) to provide the title compound as a solid (39.3 mg, 59%). ¹H NMR (400 MHz, DMSO) δ 13.13 (s, 1H), 10.87 (s, 1H), 8.79 (dd, J=2.5, 0.7 Hz, 1H), 8.15 (dd, J=9.1, 2.5 Hz, 1H), 7.77-7.68 (m, 1H), 7.60-7.50 (m, 1H), 7.55 (s, 1H), 7.32-7.19 (m, 4H), 6.93 (d, J=9.1 Hz, 1H), 6.89-6.81 (m, 2H), 6.04 (s, 2H), 4.69-4.43 (m, 2H), 4.02-3.80 (m, 1H), 3.69 (s, 3H), 3.07-2.86 (m, 2H), 2.95 (s, 3H), 2.67 (s, 3H), 1.85-1.54 (m, 4H). m/z (ES⁺), [M+H]⁺ 615.6. HPLC (A05) t_{R}=2.45 min.

### Step 2: N-[5-(1H-benzimidazol-2-yl)-1H-pyrazol-3-yl]-6-[4-[methyl(methyl-sulfonyl)amino]-1-piperidyl]pyridine-3-carboxamide

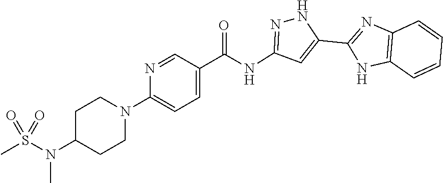 [SMILES: CN(C1CCN(c2ccc(C(=O)Nc3cc(-c4nc5ccccc5[nH]4)[nH]n3)cn2)CC1)S(C)(=O)=O]

TFA (1 mL) was added to N-[5-(1H-benzimidazol-2-yl)-1-[(4-methoxyphenyl)methyl]pyrazol-3-yl]-6-[4-[methyl(methylsulfonyl)amino]-1-piperidyl]pyridine-3-carboxamide (35 mg, 0.0569 mmol), and the mixture was stirred at 23° C. for 45 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL), and purified by preparative HPLC (BEH C18 30×150) eluting with water (10 mM (NH₄)(HCO₃)) and MeCN (38-48%) to provide the title compound as a solid (10.8 mg, 38%). ¹H NMR (500 MHz, DMSO) δ 8.80 (d, J=2.5 Hz, 1H), 8.15 (dd, J=9.0, 2.5 Hz, 1H), 7.60-7.54 (m, 2H), 7.23-7.17 (m, 2H), 7.17-7.08 (m, 1H), 6.95 (d, J=9.0 Hz, 1H), 4.66-4.47 (m, 2H), 4.02-3.82 (m, 1H), 3.05-2.88 (m, 2H), 2.96 (s, 3H), 2.68 (s, 3H), 1.86-1.55 (m, 4H). m/z (ES⁺), [M+H]⁺ 495.6. HPLC (A05) t_{R}=2.06 min.

### Preparation of Compound A-170.

### Step 1: 6-[4-[acetyl(methyl)amino]-1-piperidyl]-N-[5-(1H-benzimidazol-2-yl)-1-[(4-methoxyphenyl)methyl]pyrazol-3-yl]pyridine-3-carboxamide

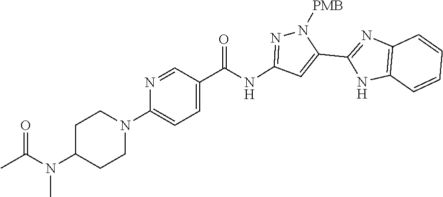

N-[5-(1H-Benzimidazol-2-yl)-1-[(4-methoxyphenyl)methyl]pyrazol-3-yl]-6-chloro-pyridine-3-carboxamide (L-1) (50 mg, 0.109 mmol) was dissolved in DMSO (1 mL). DIEA (38.0 μL, 0.218 mmol) and N-methyl-N-(4-piperidyl)acetamide (25.5 mg, 0.163 mmol) were added. The mixture was heated at 100° C. for 18 h. Water (15 mL) was added, and the mixture was stirred at 23° C. for 5 min. The mixture was filtered, and the solid was purified by silica gel chromatography (12 g cartridge) eluting with DCM and MeOH (0-10%) to provide the title compound as a solid in a a 2:1 ratio of rotamers (42.5 mg, 67%). ¹H NMR (400 MHz, DMSO) δ 13.13 (s, 1H), 10.87 (s, 1H), 8.79 (d, J=2.5 Hz, 1H), 8.14 (dd, J=9.1, 2.4 Hz, 1H), 7.78-7.67 (m, 1H), 7.61-7.48 (m, 1H), 7.55 (s, 1H), 7.33-7.16 (m, 4H), 7.00-6.88 (m, 1H), 6.90-6.82 (m, 2H), 6.04 (s, 2H), 4.67-4.46 (m, 3H), 3.69 (s, 3H), 3.04-2.89 (m, 2H), 2.78 [2.63] (s, 3H), 2.00 [2.11] (s, 3H), 1.77-1.49 (m, 4H). m/z (ES⁺), [M+H]⁺ 579.8. HPLC (A05) t_{R}=2.36 min.

### Step 2: 6-[4-[acetyl(methyl)amino]-1-piperidyl]-N-[5-(1H-benzimidazol-2-yl)-1H-pyrazol-3-yl]pyridine-3-carboxamide

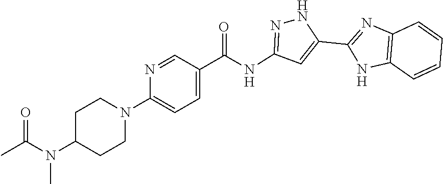 [SMILES: CC(=O)N(C)C1CCN(c2ccc(C(=O)Nc3cc(-c4nc5ccccc5[nH]4)[nH]n3)cn2)CC1]

TFA (1 mL) was added to 6-[4-[acetyl(methyl)amino]-1-piperidyl]-N-[5-(1H-benzimidazol-2-yl)-1-[(4-methoxyphenyl)methyl]pyrazol-3-yl]pyridine-3-carboxamide (41.6 mg, 0.0719 mmol), and the mixture was stirred at 23° C. for 45 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL) and dried under high vacuum to provide the title compound as a solid (24.7 mg, 75%). ¹H NMR (400 MHz, DMSO) δ 13.74-13.30 (m, 1H), 13.18-12.71 (m, 1H), 11.09-10.54 (m, 1H), 9.22-8.47 (m, 1H), 8.34-8.02 (m, 1H), 7.83-7.33 (m, 3H), 7.33-7.13 (m, 2H), 7.13-6.83 (m, 1H), 5.01-4.07 (m, 3H), 3.08-2.88 (m, 2H), 2.78 [2.64] (s, 3H), 2.01 [2.11] (s, 3H), 1.86-1.38 (m, 4H). m/z (ES⁺), [M+H]⁺ 459.7. HPLC (A05) t_{R}=1.98 min.

### Preparation of Compound A-171.

### Step 1: N-[5-(1H-benzimidazol-2-yl)-1-[(4-methoxyphenyl)methyl]pyrazol-3-yl]-6-[4-(oxetan-3-yl)piperazin-1-yl]pyridine-3-carboxamide

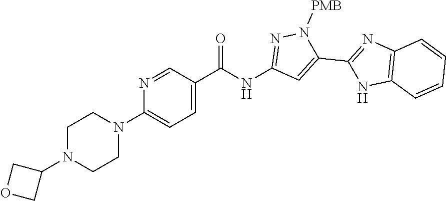

1-(Oxetan-3-yl)piperazine (0.0228 g, 0.160 mmol) was added to a solution of N-[5-(1H-benzimidazol-2-yl)-1-[(4-methoxyphenyl)methyl]pyrazol-3-yl]-6-chloro-pyridine-3-carboxamide (L-1) (70 mg, 0.153 mmol) and DIEA (0.0522 mL, 0.305 mmol) in DMSO (1 mL) The mixture was stirred at 110° C. for 18 h. Water (20 mL) was added, and the mixture was filtered. The solid was purified by silica gel chromatography (12 g cartridge) eluting with DCM and MeOH (0-10%) to provide the title compound as a solid (48.6 mg, 56%). ¹H NMR (400 MHz, DMSO) δ 13.12 (s, 1H), 10.88 (s, 1H), 8.79 (d, J=2.5 Hz, 1H), 8.14 (dd, J=9.0, 2.5 Hz, 1H), 7.72 (dd, J=23.4, 9.5 Hz, 1H), 7.56-7.51 (m, 2H), 7.28-7.20 (m, 4H), 6.89 (d, J=9.3 Hz, 1H), 6.87-6.83 (m, 2H), 6.02 (s, 2H), 4.56 (t, J=6.5 Hz, 2H), 4.48 (t, J=6.1 Hz, 2H), 3.68 (s, 3H), 3.67-3.63 (m, 4H), 3.48-3.40 (m, 1H), 2.36-2.32 (m, 4H). m/z (ES⁺), [M+H]⁺ 565.1. HPLC (A05) t_{R}=2.31 min.

### Step 2: N-[5-(1H-benzimidazol-2-yl)-1H-pyrazol-3-yl]-6-[4-(oxetan-3-yl)-piperazin-1-yl]pyridine-3-carboxamide

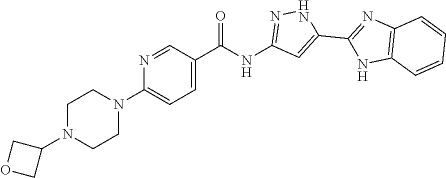 [SMILES: O=C(Nc1cc(-c2nc3ccccc3[nH]2)[nH]n1)c1ccc(N2CCN(C3COC3)CC2)nc1]

TFA (1 mL) was added to N-[5-(1H-benzimidazol-2-yl)-1-[(4-methoxyphenyl)methyl]pyrazol-3-yl]-6-[4-(oxetan-3-yl)piperazin-1-yl]pyridine-3-carboxamide (48 mg, 0.0850 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The solid was purified by preparative HPLC (BEH C18 30×150) eluting with water (10 mM (NH₄)(HCO₃)) and MeCN (25-35%) to provide the title compound as a solid (18.6 mg, 49%). ¹H NMR (400 MHz, DMSO) δ 8.80 (d, J=2.5 Hz, 1H), 8.15 (dd, J=9.0, 2.5 Hz, 1H), 7.62-7.54 (m, 2H), 7.32-7.14 (dd, J=5.8, 3.1 Hz, 3H), 6.92 (d, J=9.1 Hz, 1H), 4.57 (t, J=6.5 Hz, 2H), 4.49 (t, J=6.1 Hz, 2H), 3.70-3.64 (m, 4H), 3.45 (dt, J=12.7, 6.3 Hz, 1H), 2.39-2.33 (m, 4H). m/z (ES⁺), [M+H]⁺ 445.2. HPLC (A05) t_{R}=1.88 min.

### Preparation of Compound A-172.

### Step 1: N-[5-(1H-benzimidazol-2-yl)-1-[(4-methoxyphenyl)methyl]pyrazol-3-yl]-6-(4-morpholino-1-piperidyl)pyridine-3-carboxamide

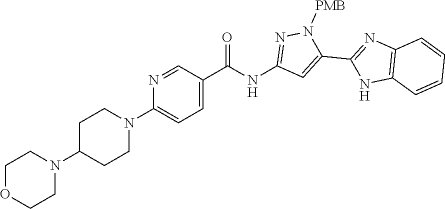

4-(4-Piperidyl)morpholine (0.0273 g, 0.160 mmol) was added to a solution of N-[5-(1H-benzimidazol-2-yl)-1-[(4-methoxyphenyl)methyl]pyrazol-3-yl]-6-chloro-pyridine-3-carboxamide (L-1) (70 mg, 0.153 mmol) and DIEA (0.0522 mL, 0.305 mmol) in DMSO (1 mL). The mixture was stirred at 100° C. for 18 h. Water (20 mL) was added, and the mixture was filtered. The solid was purified by silica gel chromatography (12 g cartridge) eluting with DCM and MeOH (0-10%) to provide the title compound as a solid (44.4 mg, 49%). ¹H NMR (400 MHz, DMSO) δ 13.08 (s, 1H), 10.81 (s, 1H), 8.74 (d, J=2.8 Hz, 1H), 8.08 (dd, J=9.1, 2.5 Hz, 1H), 7.68 (d, J=6.3 Hz, 1H), 7.53-7.47 (m, 2H), 7.25-7.17 (m, 4H), 6.86-6.80 (m, 3H), 5.99 (s, 2H), 4.40 (d, J=13.0 Hz, 2H), 3.65 (s, 3H), 3.55-3.50 (m, 4H), 2.87 (t, J=13.1 Hz, 2H), 2.45-2.36 (m, 5H), 1.82 (d, J=11.1 Hz, 2H), 1.31 (qd, J=12.4, 4.0 Hz, 2H). m/z (ES⁺), [M+H]⁺ 593.4. HPLC (A05) t_{R}=2.14 min.

### Step 2: N-[5-(1H-benzimidazol-2-yl)-1H-pyrazol-3-yl]-6-(4-morpholino-1-piperidyl)pyridine-3-carboxamide

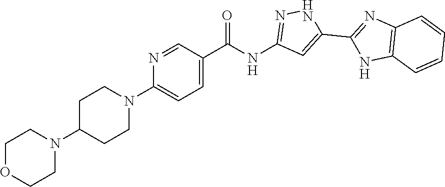 [SMILES: O=C(Nc1cc(-c2nc3ccccc3[nH]2)[nH]n1)c1ccc(N2CCC(N3CCOCC3)CC2)nc1]

TFA (1 mL) was added to N-[5-(1H-benzimidazol-2-yl)-1-[(4-methoxyphenyl)methyl]pyrazol-3-yl]-6-(4-morpholino-1-piperidyl)pyridine-3-carboxamide (44 mg, 0.0742 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The residue was purified by preparative HPLC (BEH C18 30×150) eluting with water (10 mM (NH₄)(HCO₃)) and MeCN (27-37%) to provide the title compound as a solid (20.6 mg, 59%). ¹H NMR (400 MHz, DMSO) δ 13.29-12.56 (m, 2H), 10.79 (s, 1H), 8.74 (d, J=2.5 Hz, 1H), 8.09 (dd, J=9.1, 2.5 Hz, 1H), 7.54 (s, 2H), 7.30-7.00 (m, 3H), 6.88 (d, J=9.2 Hz, 1H), 4.41 (d, J=13.0 Hz, 2H), 3.56-3.48 (m, 4H), 2.92-2.86 (m, 2H), 2.46-2.36 (m, 5H), 1.84-1.82 (m, 2H), 1.37-1.27 (m, 2H). m/z (ES⁺), [M+H]⁺ 473.3. HPLC (A05) t_{R}=1.78 min.

### Preparation of Compound A-177.

### Step 1: N-[5-(1H-benzimidazol-2-yl)-1-[(4-methoxyphenyl)methyl]pyrazol-3-yl]-6-(4-methylpiperazin-1-yl)pyridazine-3-carboxamide

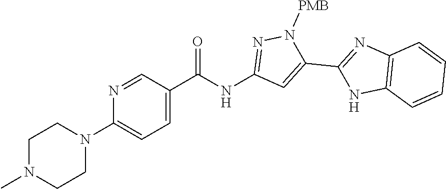

1-Methylpiperazine (0.0186 mL, 0.167 mmol) was added to a solution of N-[5-(1H-benzimidazol-2-yl)-1-[(4-methoxyphenyl)methyl]pyrazol-3-yl]-6-chloro-pyridazine-3-carboxamide (L-22) (70 mg, 0.152 mmol) and DIEA (0.0521 mL, 0.304 mmol) in DMSO (1 mL) The mixture was stirred at 100° C. for 18 h. Water (20 mL) was added, and the mixture was filtered. The solid was purified by silica gel chromatography (12 g cartridge) eluting with DCM and MeOH (0-10%) to provide the title compound as a solid (70.5 mg, 88%). ¹H NMR (400 MHz, DMSO) δ 13.14 (s, 1H), 10.60 (s, 1H), 7.95 (d, J=9.6 Hz, 1H), 7.77-7.68 (m, 1H), 7.52 (s, 2H), 7.41 (d, J=9.7 Hz, 1H), 7.25 (d, J=8.7 Hz, 4H), 6.84 (d, J=8.8 Hz, 2H), 6.02 (s, 2H), 3.78-3.70 (m, 4H), 3.68 (s, 3H), 2.47-2.42 (m, 4H), 2.24 (s, 3H). m/z (ES⁺), [M+H]⁺ 524.3. HPLC (A05) t_{R}=2.07 min.

### Step 2: N-[5-(1H-benzimidazol-2-yl)-1H-pyrazol-3-yl]-6-(4-methylpiperazin-1-yl)pyridazine-3-carboxamide

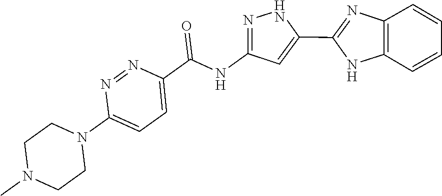 [SMILES: CN1CCN(c2ccc(C(=O)Nc3cc(-c4nc5ccccc5[nH]4)[nH]n3)nn2)CC1]

A mixture of N-[5-(1H-benzimidazol-2-yl)-1-[(4-methoxyphenyl)methyl]pyrazol-3-yl]-4-(2-hydroxyethoxy)-3-methoxy-benzamide (50 mg, 0.0974 mmol) in TFA (1 mL) was stirred at 23° C. for 30 min and concentrated. The residue was diluted in DMSO (0.500 mL) and NEt₃ (0.500 mL), and the mixture was stirred at 23° C. for 5 min. The mixture was diluted with water (10 mL). The mixture was filtered, and the solid was washed with acetone (5 mL), DCM (5 mL), and acetone (5 mL) to provide the title compound as a solid (9.00 mg, 23%). ¹H NMR (500 MHz, DMSO) δ 13.46 (s, 2H), 10.96 (s, 1H), 7.61 (s, 4H), 7.26 (s, 2H), 7.06 (s, 1H), 4.75 (s, 1H), 4.01 (s, 2H), 3.81 (s, 3H), 3.69 (s, 2H). m/z (ES⁺), [M+H]⁺ 394.2. HPLC (B30) t_{R}=1.04 min.

### Preparation of Compound A-179.

### Step 1: N-[5-(1H-benzimidazol-2-yl)-1-[(4-methoxyphenyl)methyl]pyrazol-3-yl]-5-(4-methylpiperazin-1-yl)pyrazine-2-carboxamide

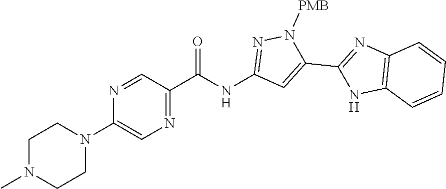

1-Methylpiperazine (0.0186 mL, 0.167 mmol) was added to a solution of N-[5-(1H-benzimidazol-2-yl)-1-[(4-methoxyphenyl)methyl]pyrazol-3-yl]-5-chloro-pyrazine-2-carboxamide (L-23) (70 mg, 0.152 mmol) and DIEA (0.0521 mL, 0.304 mmol) in DMSO (1 mL). The mixture was stirred at 100° C. for 18 h. Water (20 mL) was added, and the mixture was filtered. The solid was purified by silica gel chromatography (12 g cartridge) eluting with DCM and MeOH (0-15%) to provide the title compound as a solid (75.6 mg, 95%). ¹H NMR (400 MHz, DMSO) δ 13.14 (s, 1H), 10.17 (s, 1H), 8.74 (d, J=1.3 Hz, 1H), 8.35 (d, J=1.3 Hz, 1H), 7.71 (dt, J=12.5, 3.6 Hz, 1H), 7.54 (d, J=7.9 Hz, 1H), 7.51 (s, 1H), 7.31-7.17 (m, 4H), 6.86-6.81 (m, 2H), 6.01 (s, 2H), 3.74 (s, 4H), 3.68 (s, 3H), 2.45-2.39 (m, 4H), 2.23 (s, 3H). m/z (ES⁺), [M+H]⁺ 524.1. HPLC (A05) t_{R}=2.11 min.

### Step 2: N-[5-(1H-benzimidazol-2-yl)-1H-pyrazol-3-yl]-5-(4-methylpiperazin-1-yl)pyrazine-2-carboxamide

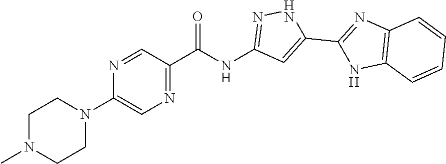 [SMILES: CN1CCN(c2cnc(C(=O)Nc3cc(-c4nc5ccccc5[nH]4)[nH]n3)cn2)CC1]

TFA (1 mL) was added to N-[5-(1H-benzimidazol-2-yl)-1-[(4-methoxyphenyl)methyl]pyrazol-3-yl]-5-(4-methylpiperazin-1-yl)pyrazine-2-carboxamide (75.6 mg, 0.144 mmol), and the mixture was stirred at 23° C. for 1 h. The mixture was concentrated. The residue was purified by preparative HPLC (BEH C18 30×150) eluting with water (10 mM (NH₄)(HCO₃)) and MeCN (28-38%) to provide the title compound as a solid (22.6 mg, 39%). ¹H NMR (400 MHz, DMSO) δ 8.76 (d, J=1.3 Hz, 1H), 8.37 (d, J=1.3 Hz, 1H), 7.56 (dd, J=5.8, 3.2 Hz, 2H), 7.24-7.07 (m, 3H), 3.78-3.72 (m, 4H), 2.46-2.40 (m, 4H), 2.24 (s, 3H). m/z (ES⁺), [M+H]⁺ 404.2. HPLC (A05) t_{R}=1.76 min.

### Preparation of Compound A-180.

### Step 1: N-[5-(1H-benzimidazol-2-yl)-1-[(4-methoxyphenyl)methyl]pyrazol-3-yl]-2-(4-methylpiperazin-1-yl)pyrimidine-5-carboxamide

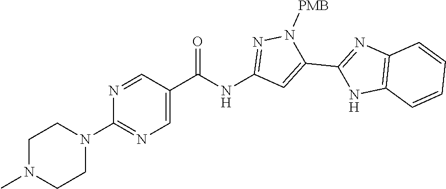

1-Methylpiperazine (0.0186 mL, 0.167 mmol) was added to a solution of N-[5-(1H-benzimidazol-2-yl)-1-[(4-methoxyphenyl)methyl]pyrazol-3-yl]-5-chloro-pyrazine-2-carboxamide (L-24) (70 mg, 0.152 mmol) and DIEA (0.0521 mL, 0.304 mmol) in DMSO (1 mL) The mixture was stirred at 100° C. for 18 h. Water (20 mL) was added, and the mixture was filtered. The solid was purified by silica gel chromatography (12 g cartridge) eluting with DCM and MeOH (0-15%) to provide the title compound as a solid (58.4 mg, 73%). ¹H NMR (400 MHz, DMSO) δ 13.13 (s, 1H), 11.02 (s, 1H), 8.94 (s, 2H), 7.72 (d, J=7.4 Hz, 1H), 7.57-7.49 (m, 2H), 7.32-7.20 (m, 4H), 6.87-6.83 (m, 2H), 6.03 (s, 2H), 3.87-3.79 (m, 4H), 3.68 (s, 3H), 2.40-2.34 (m, 4H), 2.22 (s, 3H). m/z (ES⁺), [M+H]⁺ 524.1. HPLC (A05) t_{R}=2.08 min.

### Step 2: N-[5-(1H-benzimidazol-2-yl)-1H-pyrazol-3-yl]-2-(4-methylpiperazin-1-yl)pyrimidine-5-carboxamide

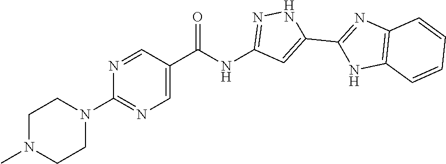 [SMILES: CN1CCN(c2ncc(C(=O)Nc3cc(-c4nc5ccccc5[nH]4)[nH]n3)cn2)CC1]

TFA (2 mL) was added to N-[5-(1H-benzimidazol-2-yl)-1-[(4-methoxyphenyl)methyl]pyrazol-3-yl]-2-(4-methylpiperazin-1-yl)pyrimidine-5-carboxamide (58.4 mg, 0.112 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (20 mL) was added, and the suspension was spun by centrifuge. The supernatant was discarded. The centrifugation process was repeated with water (20 mL), acetone (10 mL) and water (20 mL) to provide the title compound as a solid (38.3 mg, 85%). ¹H NMR (400 MHz, DMSO) δ 13.49 [13.15] (s, 1H), 13.02 [12.73] (s, 1H), [11.03] 10.94 (s, 1H), 8.96 (s, 2H), 7.67 [7.62] (d, J=7.6 Hz, 1H), 7.53 [7.47] (d, J=7.1 Hz, 1H), 7.41 (s, 1H), 7.27-7.14 (m, 2H), 4.01-3.75 (m, 4H), 2.49-2.36 (m, 4H), 2.27 (s, 3H). m/z (ES⁺), [M+H]⁺ 404.4. HPLC (A05) t_{R}=1.74 min.

### Preparation of Compound A-183.

### Step 1: N-[5-(1H-benzimidazol-2-yl)-1-[(4-methoxyphenyl)methyl]pyrazol-3-yl]-2-(3-methoxyazetidin-1-yl)pyrimidine-5-carboxamide

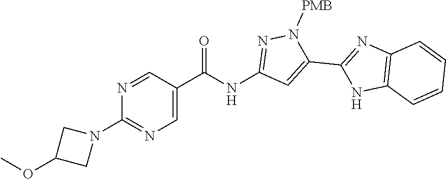

3-Methoxyazetidine hydrochloride (20.7 mg, 0.167 mmol) was added to a solution of N-[5-(1H-benzimidazol-2-yl)-1-[(4-methoxyphenyl)methyl]pyrazol-3-yl]-2-chloro-pyrimidine-5-carboxamide (L-24) (70 mg, 0.152 mmol) and DIEA (0.0782 mL, 0.457 mmol) in DMSO (1 mL). The mixture was stirred at 100° C. for 18 h. Water (20 mL) was added, and the mixture was filtered. The solid was purified by silica gel chromatography (12 g cartridge) eluting with DCM and MeOH (0-10%) to provide the title compound as a solid (58 mg, 75%). ¹H NMR (400 MHz, DMSO) δ 13.13 (s, 1H), 11.04 (s, 1H), 8.92 (s, 2H), 7.72 (d, J=7.1 Hz, 1H), 7.56-7.49 (m, 2H), 7.30-7.20 (m, 4H), 6.87-6.83 (m, 2H), 6.03 (s, 2H), 4.35-4.29 (m, 3H), 3.95-3.92 (m, 2H), 3.68 (s, 3H), 3.27 (s, 3H). m/z (ES⁺), [M+H]⁺ 511.2. HPLC (A05) t_{R}=2.33 min.

### Step 2: N-[5-(1H-benzimidazol-2-yl)-1H-pyrazol-3-yl]-2-(3-methoxyazetidin-1-yl)pyrimidine-5-carboxamide

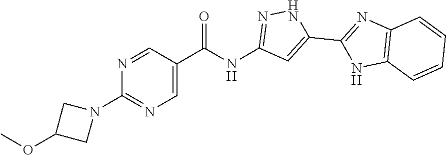 [SMILES: COC1CN(c2ncc(C(=O)Nc3cc(-c4nc5ccccc5[nH]4)[nH]n3)cn2)C1]

TFA (2 mL) was added to N-[5-(1H-benzimidazol-2-yl)-1-[(4-methoxyphenyl)methyl]pyrazol-3-yl]-2-(3-methoxyazetidin-1-yl)pyrimidine-5-carboxamide (58 mg, 0.114 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide the title compound as a solid in a 4:1 mixture of tautomers (27.7 mg, 62%). ¹H NMR (400 MHz, DMSO) δ 13.49 [13.15] (s, 1H), 13.02 [12.73] (s, 1H), [11.04] 10.96 (s, 1H), 8.94 [8.91] (s, 2H), 7.67 [7.62] (d, J=7.0 Hz, 1H), 7.53 [7.46] (d, J=6.8 Hz, 1H), 7.40 [6.64] (d, J=2.1 Hz, 1H), 7.28-7.13 (m, 2H), 4.39-4.30 (m, 3H), 4.00-3.91 (m, 2H), 3.27 (s, 3H). m/z (ES⁺), [M+H]⁺ 391.1. HPLC (A05) t_{R}=1.86 min.

### Preparation of Compound A-186.

### Step 1: N-[5-(1H-benzimidazol-2-yl)-1-[(4-methoxyphenyl)methyl]pyrazol-3-yl]-5-(3-methoxyazetidin-1-yl)pyrazine-2-carboxamide

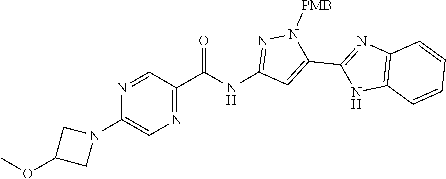

3-Methoxyazetidine hydrochloride (20.7 mg, 0.167 mmol) was added to a solution of N-[5-(1H-benzimidazol-2-yl)-1-[(4-methoxyphenyl)methyl]pyrazol-3-yl]-5-chloro-pyrazine-2-carboxamide (L-23) (70 mg, 0.152 mmol) and DIEA (0.0782 mL, 0.457 mmol) in DMSO (1 mL). The mixture was stirred at 100° C. for 18 h. Water (20 mL) was added, and the mixture was filtered. The solid was purified by silica gel chromatography (12 g cartridge) eluting with DCM and MeOH (0-10%) to provide the title compound as a solid (58 mg, 75%). ¹H NMR (400 MHz, DMSO) δ 13.14 (s, 1H), 10.18 (s, 1H), 8.72 (d, J=1.4 Hz, 1H), 7.90 (d, J=1.4 Hz, 1H), 7.73 (d, J=7.4 Hz, 1H), 7.54 (d, J=7.8 Hz, 1H), 7.51 (s, 1H), 7.27 (dd, J=19.2, 7.4 Hz, 4H), 6.84 (d, J=8.8 Hz, 2H), 6.01 (s, 2H), 4.43-4.33 (m, 3H), 4.04-3.96 (m, 2H), 3.68 (s, 3H), 3.28 (s, 3H). m/z (ES⁺), [M+H]⁺ 511.2. HPLC (A05) t_{R}=2.49 min.

### Step 2: N-[5-(1H-benzimidazol-2-yl)-1H-pyrazol-3-yl]-5-(3-methoxyazetidin-1-yl)pyrazine-2-carboxamide

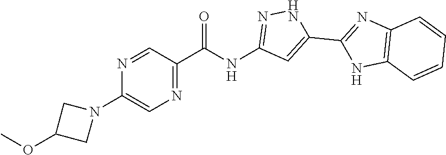 [SMILES: COC1CN(c2cnc(C(=O)Nc3cc(-c4nc5ccccc5[nH]4)[nH]n3)cn2)C1]

TFA (2 mL) was added to N-[5-(1H-benzimidazol-2-yl)-1-[(4-methoxyphenyl)methyl]pyrazol-3-yl]-5-(3-methoxyazetidin-1-yl)pyrazine-2-carboxamide (58 mg, 0.114 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The residue was purified by preparative HPLC (BEH C18 30×150) eluting with water (10 mM (NH₄)(HCO₃)) and MeCN (28-38%) to provide the title compound as a solid (17.1 mg, 39%). ¹H NMR (400 MHz, DMSO) δ 12.88 (br, 2H), 10.69 (br, 1H), 8.71 (d, J=1.4 Hz, 1H), 7.89 (d, J=1.4 Hz, 1H), 7.60-7.45 (m, 2H), 7.24-7.00 (m, 3H), 4.40-4.33 (m, 3H), 4.01-3.95 (m, 2H), 3.25 (s, 3H). m/z (ES⁺), [M+H]⁺ 391.6. HPLC (A05) t_{R}=1.84 min.

### Preparation of Compound A-188.

### Step 1: N-[5-(7-fluoro-5-methoxy-1H-benzimidazol-2-yl)-1-[(4-methoxyphenyl)methyl]pyrazol-3-yl]-6-(3-methoxyazetidin-1-yl)pyridine-3-carboxamide

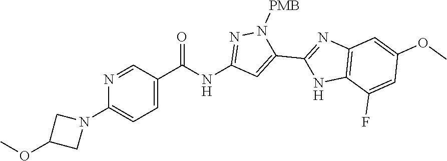

3-Methoxyazetidine hydrochloride (19.5 mg, 0.158 mmol) was added to a solution of 6-chloro-N-[5-(7-fluoro-5-methoxy-1H-benzimidazol-2-yl)-1-[(4-methoxyphenyl)methyl]pyrazol-3-yl]-pyridine-3-carboxamide (L-10) (80 mg, 0.158 mmol) and DIEA (0.0810 mL, 0.473 mmol) in DMSO (1.50 mL) The mixture was stirred at 100° C. for 18 h. Water (20 mL) was added, and the mixture was filtered. The solid was purified by silica gel chromatography (12 g cartridge) eluting with hexanes and EtOAc (0-100%) to provide the title compound as a solid (24 mg, 27%). ¹H NMR (400 MHz, DMSO) δ 13.25 (s, 1H), 10.88 (s, 1H), 8.76 (d, J=2.3 Hz, 1H), 8.13 (dd, J=8.8, 2.4 Hz, 1H), 7.50 (s, 1H), 7.24 (d, J=8.6 Hz, 2H), 6.89-6.81 (m, 3H), 6.75 (dd, J=12.5, 2.1 Hz, 1H), 6.42 (d, J=8.8 Hz, 1H), 5.96 (s, 2H), 4.35 (tt, J=6.5, 3.4 Hz, 1H), 4.23 (dd, J=9.0, 6.8 Hz, 2H), 3.88-3.82 (m, 5H), 3.69 (s, 3H), 3.26 (s, 3H). m/z (ES⁺), [M+H]⁺ 558.3. HPLC (A05) t_{R}=2.38 min.

### Step 2: N-[5-(7-fluoro-5-methoxy-1H-benzimidazol-2-yl)-1H-pyrazol-3-yl]-6-(3-methoxyazetidin-1-yl)pyridine-3-carboxamide

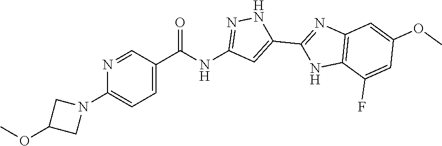

TFA (1 mL) was added to N-[5-(7-fluoro-5-methoxy-1H-benzimidazol-2-yl)-1-[(4-methoxyphenyl)methyl]pyrazol-3-yl]-6-(3-methoxyazetidin-1-yl)pyridine-3-carboxamide (24 mg, 0.0430 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The solid was purified by preparative HPLC (BEH C18 30×100) eluting with water (10 mM (NH₄)(HCO₃)) and MeCN (29-39%) to provide the title compound as a solid (4.90 mg, 26%). ¹H NMR (400 MHz, DMSO) δ 13.10 (br, 2H), 10.83 (s, 1H), 8.71 (d, J=2.2 Hz, 1H), 8.11 (d, J=8.6 Hz, 1H), 7.32-6.99 (m, 1H), 6.81 (s, 1H), 6.69 (d, J=10.8 Hz, 1H), 6.45 (d, J=8.5 Hz, 1H), 4.33 (dq, J=7.7, 3.8 Hz, 1H), 4.26-4.19 (m, 2H), 3.85 (dd, J=9.7, 3.7 Hz, 2H), 3.79 (s, 3H), 3.23 (s, 3H). m/z (ES⁺), [M+H]⁺ 438.5. HPLC (A05) t_{R}=2.08 min.

### Preparation of Compound A-192.

### Step 1: N-[5-(7-fluoro-5-methoxy-1H-benzimidazol-2-yl)-1-[(4-methoxyphenyl)-methyl]pyrazol-3-yl]-6-[4-(2-methoxyethyl)piperazin-1-yl]pyridine-3-carboxamide.

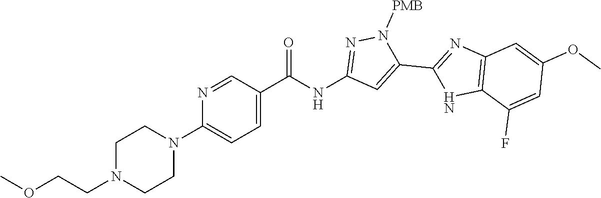

1-(2-MethoxyethMl)piperazine (0.0235 mL, 0.158 mmol) was added to a solution of 6-chloro-N-[5-(7-fluoro-5-methoxy-1H-benzimidazol-2-yl)-1-[(4-methoxyphenyl)methyl]pyrazol-3-yl]-pyridine-3-carboxamide (L-10) (80 mg, 0.158 mmol) and DIEA (0.0540 mL, 0.316 mmol) in DMSO (1.50 mL) The mixture was stirred at 100° C. for 18 h. EtOAc (150 mL) was added, and the organic phase was washed with water (4×50 mL) and brine (50 mL), dried (Na₂SO₄), filtered, and concentrated. The residue was purified by silica gel chromatography (12 g cartridge) eluting with DCM and MeOH (0-10%) to provide the title compound as a solid (72 mg, 74%). ¹H NMR (500 MHz, DMSO) δ 13.24 (s, 1H), 10.87 (s, 1H), 8.79 (d, J=2.5 Hz, 1H), 8.14 (dd, J=9.1, 2.5 Hz, 1H), 7.51 (s, 1H), 7.24 (d, J=8.4 Hz, 2H), 6.88-6.83 (m, 4H), 6.75 (d, J=10.6 Hz, 1H), 5.97 (s, 2H), 3.84 (s, 3H), 3.70 (s, 3H), 3.63-3.59 (m, 4H), 3.48 (t, J=5.7 Hz, 2H), 3.26 (s, 3H), 2.54-2.52 (m, J=4.7, 3.0 Hz, 6H). ¹⁹F NMR (471 MHz, DMSO) δ −127.12 (s). m/z (ES⁺), [M+H]⁺ 615.8. HPLC (A05) t_{R}=2.06 min.

### Step 2: N-[5-(7-fluoro-5-methoxy-1H-benzimidazol-2-yl)-1H-pyrazol-3-yl]-6-[4-(2-methoxyethyl)piperazin-1-yl]pyridine-3-carboxamide

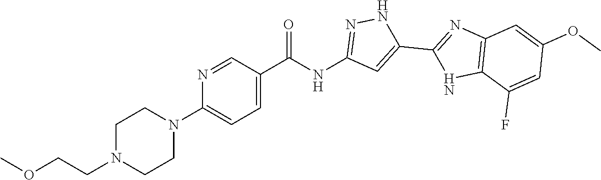

TFA (1.50 mL) was added to N-[5-(1H-benzimidazol-2-yl)-1-[(4-methoxyphenyl)methyl]pyrazol-3-yl]-2-(3-methoxyazetidin-1-yl)pyrimidine-5-carboxamide (58 mg, 0.114 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The residue was purified by preparative HPLC (BEH C18 30×150) eluting with water (10 mM (NH₄)(HCO₃)) and MeCN (32-42%) to provide the title compound as a solid in a mixture of tautomers (15.6 mg, 27%). ¹H NMR (500 MHz, DMSO) δ 13.41 [13.36] [13.28] (s, 1H), 13.08 [13.05] [12.81] (s, 1H), [10.86] 10.73 [9.75] (s, 1H), [8.79] 8.73 (s, 1H), 8.17-8.00 (m, 1H), [7.40] 7.32 [6.53] (s, 1H), 6.96-6.82 (m, 1H), 6.78 [6.71] (s, 1H), 6.66 [6.59] (d, J=12.7 Hz, 1H), 3.76 [3.73] (s, 3H), 3.64-3.36 (m, 6H), 3.23-2.96 (m, 4H), 2.58-2.44 (m, 5H). m/z (ES⁺), [M+H]⁺ 495.5. HPLC (A05) t_{R}=1.84 min.

### Preparation of Compound A-193.

### Step 1: N-[5-(7-fluoro-5-methoxy-1H-benzimidazol-2-yl)-1-[(4-methoxyphenyl)-methyl]pyrazol-3-yl]-6-(4-methoxy-1-piperidyl)pyridine-3-carboxamide

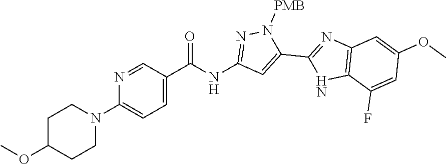

4-Methoxypiperidine (18.2 mg, 0.158 mmol) was added to a solution of 6-chloro-N-[5-(7-fluoro-5-methoxy-1H-benzimidazol-2-yl)-1-[(4-methoxyphenyl)methyl]pyrazol-3-yl]pyridine-3-carboxamide (L-10) (80 mg, 0.158 mmol) and DIEA (0.0540 mL, 0.316 mmol) in DMSO (1.50 mL) The mixture was stirred at 100° C. for 18 h. Water (20 mL) was added, and the mixture was filtered. The solid was purified by silica gel chromatography (12 g cartridge) eluting with DCM and MeOH (0-5%) to provide the title compound as a solid (72 mg, 78%). ¹H NMR (400 MHz, DMSO) δ 13.24 (s, 1H), 10.85 (s, 1H), 8.77 (d, J=2.5 Hz, 1H), 8.12 (dd, J=9.1, 2.5 Hz, 1H), 7.51 (s, 1H), 7.24 (d, J=8.7 Hz, 2H), 6.91-6.82 (m, 4H), 6.75 (d, J=12.4 Hz, 1H), 5.96 (s, 2H), 4.03 (dt, J=9.4, 4.5 Hz, 2H), 3.83 (s, 3H), 3.69 (s, 3H), 3.49-3.40 (m, 1H), 3.35-3.29 (m, 2H), 3.28 (s, 3H), 1.93-1.85 (m, 2H), 1.46-1.35 (m, J=12.6, 9.0, 4.5 Hz, 2H). ¹⁹F NMR (376 MHz, DMSO) δ −127.22 (s). m/z (ES⁺), [M+H]⁺ 586.4. HPLC (A05) t_{R}=2.56 min.

### Step 2: N-[5-(7-fluoro-5-methoxy-1H-benzimidazol-2-yl)-1H-pyrazol-3-yl]-6-(4-methoxy-1-piperidyl)pyridine-3-carboxamide

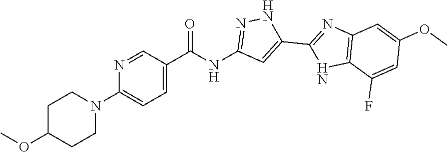

TFA (1.50 mL) was added to N-[5-(7-fluoro-5-methoxy-1H-benzimidazol-2-yl)-1-[(4-methoxyphenyl)methyl]pyrazol-3-yl]-6-(4-methoxy-1-piperidyl)pyridine-3-carboxamide (72 mg, 0.123 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The solid was purified by preparative HPLC (BEH C18 30×150) eluting with water (10 mM (NH₄)(HCO₃)) and MeCN (34-44%) to provide the title compound as a solid (18.4 mg, 32%). ¹H NMR (400 MHz, DMSO) δ 13.21 (s, 2H), 10.84 (s, 1H), 8.78 (d, J=2.4 Hz, 1H), 8.12 (dd, J=9.1, 2.5 Hz, 1H), 7.13 (s, 1H), 6.92 (d, J=9.2 Hz, 1H), 6.84 (s, 1H), 6.68 (d, J=12.8 Hz, 1H), 4.10-4.00 (m, 2H), 3.81 (s, 3H), 3.50-3.43 (m, 1H), 3.38-3.26 (m, 5H), 1.95-1.86 (m, 2H), 1.48-1.36 (m, 2H). m/z (ES⁺), [M+H]⁺ 466.6. HPLC (A05) t_{R}=2.18 min.

### Preparation of Compound A-194.

### Step 1: N-[5-(7-fluoro-5-methoxy-1H-benzimidazol-2-yl)-1-[(4-methoxyphenyl)-methyl]pyrazol-3-yl]-6-(4-methylpiperazin-1-yl)pyridine-3-carboxamide

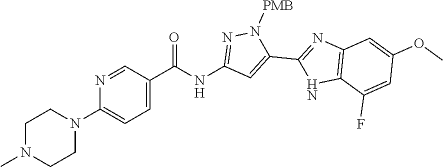

1-Methylpiperazine (0.0176 mL, 0.158 mmol) was added to a solution of 6-chloro-N-[5-(7-fluoro-5-methoxy-1H-benzimidazol-2-yl)-1-[(4-methoxyphenyl)methyl]pyrazol-3-yl]pyridine-3-carboxamide (L-10) (80 mg, 0.158 mmol) and DIEA (0.0540 mL, 0.316 mmol) in DMSO (1.50 mL). The mixture was stirred at 100° C. for 18 h. Water (20 mL) was added, and the mixture was filtered. The solid was purified by silica gel chromatography (12 g cartridge) eluting with DCM and MeOH (0-20%) to provide the title compound as a solid (61 mg, 68%). ¹H NMR (400 MHz, DMSO) δ 13.25 (s, 1H), 10.88 (s, 1H), 8.78 (d, J=2.3 Hz, 1H), 8.13 (dd, J=9.1, 2.5 Hz, 1H), 7.51 (s, 1H), 7.23 (d, J=8.5 Hz, 2H), 6.89-6.81 (m, 4H), 6.74 (d, J=13.2 Hz, 1H), 5.96 (s, 2H), 3.83 (s, 3H), 3.69 (s, 3H), 3.64-3.60 (m, 4H), 2.40-2.36 (m, 4H), 2.22 (s, 3H). ¹⁹F NMR (376 MHz, DMSO) δ −127.23 (s). m/z (ES⁺), [M+H]⁺ 571.7. HPLC (A05) t_{R}=2.04 min.

### Step 2: N-[5-(7-fluoro-5-methoxy-1H-benzimidazol-2-yl)-1H-pyrazol-3-yl]-6-(4-methylpiperazin-1-yl)pyridine-3-carboxamide

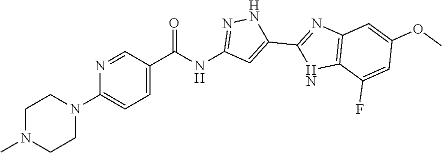

TFA (1.50 mL) was added to N-[5-(7-fluoro-5-methoxy-1H-benzimidazol-2-yl)-1-[(4-methoxyphenyl)methyl]pyrazol-3-yl]-6-(4-methylpiperazin-1-yl)pyridine-3-carboxamide (61 mg, 0.107 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The residue was purified by preparative HPLC (BEH C18 30×150) eluting with water (10 mM (NH₄)(HCO₃)) and MeCN (31-41%) to provide the title compound as a solid (30.7 mg, 64%). ¹H NMR (400 MHz, DMSO) δ 13.16 (br, 2H), 10.77 (s, 1H), 8.75 (d, J=2.4 Hz, 1H), 8.10 (dd, J=9.0, 2.5 Hz, 1H), 7.17 (s, 1H), 6.87 (d, J=9.1 Hz, 1H), 6.80 (s, 1H), 6.65 (d, J=11.0 Hz, 1H), 3.78 (s, 3H), 3.63-3.57 (m, 4H), 2.38-2.33 (m, 4H), 2.18 (s, 3H). m/z (ES⁺), [M+H]⁺ 451.8. HPLC (A05) t_{R}=1.80 min.

### Preparation of Compound A-218.

### Step 1: N-[5-(1H-benzimidazol-2-yl)-1-[(4-methoxyphenyl)methyl]pyrazol-3-yl]-6-piperazin-1-yl-pyridine-3-carboxamide

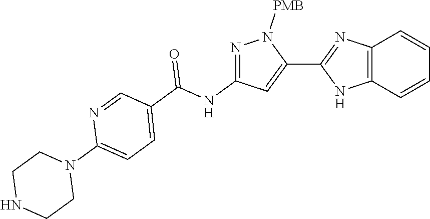

Piperazine (0.0394 g, 0.458 mmol) was added to a solution of N-[5-(1H-benzimidazol-2-yl)-1-[(4-methoxyphenyl)methyl]pyrazol-3-yl]-6-chloro-pyridine-3-carboxamide (L-1) (70 mg, 0.153 mmol) and DIEA (0.0522 mL, 0.305 mmol) in DMSO (1 mL). The mixture was stirred at 100° C. for 18 h. Water (20 mL) was added, and the mixture was filtered. The residue was purified by silica gel chromatography (12 g cartridge) eluting with DCM and MeOH (0-60%) to provide the title compound as a solid (76 mg, 98%). ¹H NMR (400 MHz, DMSO) δ 13.12 (s, 1H), 10.88 (s, 1H), 8.79 (d, J=2.5 Hz, 1H), 8.15 (dd, J=9.1, 2.5 Hz, 1H), 7.72 (d, J=7.6 Hz, 1H), 7.56-7.52 (m, 2H), 7.30-7.21 (m, 4H), 6.89-6.83 (m, 3H), 6.02 (s, 2H), 3.68 (s, 3H), 3.66-3.61 (m, 4H), 3.30-3.27 (m, J=4.2 Hz, 1H), 2.91-2.87 (m, 4H). m/z (ES⁺), [M+H]⁺ 509.5. HPLC (A05) t_{R}=2.05 min.

### Step 2: N-[5-(1H-benzimidazol-2-yl)-1H-pyrazol-3-yl]-6-piperazin-1-yl-pyridine-3-carboxamide.

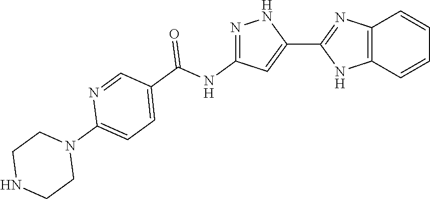 [SMILES: O=C(Nc1cc(-c2nc3ccccc3[nH]2)[nH]n1)c1ccc(N2CCNCC2)nc1]

TFA (1.50 mL) was added to N-[5-(1H-benzimidazol-2-yl)-1-[(4-methoxyphenyl)methyl]pyrazol-3-yl]-6-piperazin-1-yl-pyridine-3-carboxamide (76 mg, 0.149 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The solid was purified by preparative HPLC (BEH C18 30×150) eluting with water (10 mM (NH₄)(HCO₃)) and MeCN (20-30%) to provide the title compound as a solid (21.9 mg, 38%). ¹H NMR (400 MHz, DMSO) δ 13.51 (s, 1H), 13.03 (s, 1H), 10.85 (s, 1H), [8.81] 8.79 (d, J=2.4 Hz, 1H), [8.18] 8.13 (dd, J=9.0, 2.4 Hz, 1H), 7.70-7.60 (m, 1H), 7.58-7.46 (m, 1H), 7.40-7.07 (m, 3H), [6.94] 6.87 (d, J=9.0 Hz, 1H), [3.71-3.61] 3.59-3.51 (m, 4H), [3.50-3.44] 2.82-2.72 (m, 4H). m/z (ES⁺), [M+H]⁺ 389.7. HPLC (A05) t_{R}=1.80 min.

### Preparation of Compound A-220.

### Step 1: N-[5-(1H-benzimidazol-2-yl)-1-[(4-methoxyphenyl)methyl]pyrazol-3-yl]-4-[4-(oxetan-3-yl)piperazin-1-yl]benzamide

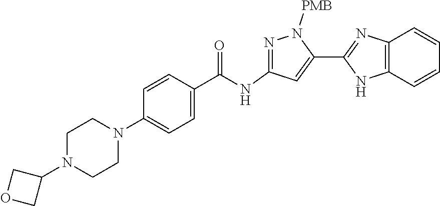

1-(Oxetan-3-yl)piperazine (161 mg, 1.13 mmol) was added to a solution of N-[5-(1H-benzimidazol-2-yl)-1-[(4-methoxyphenyl)methyl]pyrazol-3-yl]-4-fluoro-benzamide (L-15) (100 mg, 0.227 mmol) in DMSO (0.600 mL). The mixture was heated at 110° C. for 42 h. EtOAc (60 mL) was added, and the organic phase was washed with water (3×40 mL) and brine (50 mL), dried (MgSO₄), filtered, and concentrated. The residue was purified by silica gel chromatography (12 g cartridge) eluting with DCM and MeOH (0-10%) to provide the title compound as a solid (68 mg, 53%). ¹H NMR (400 MHz, DMSO) δ 13.11 (s, 1H), 10.75 (s, 1H), 7.95 (d, J=9.0 Hz, 2H), 7.71 (d, J=8.3 Hz, 1H), 7.57-7.50 (m, 2H), 7.30-7.20 (m, 4H), 6.98 (d, J=9.1 Hz, 2H), 6.87-6.83 (m, 2H), 6.02 (s, 2H), 4.57 (t, J=6.5 Hz, 2H), 4.48 (t, J=6.1 Hz, 2H), 3.68 (s, 3H), 3.48-3.41 (m, 1H), 3.35-3.31 (m, 4H), 2.41-2.38 (m, 4H). m/z (ES⁺), [M+H]⁺ 564.7. HPLC (A05) t_{R}=2.35 min.

### Step 2: N-[5-(1H-benzimidazol-2-yl)-1H-pyrazol-3-yl]-4-[4-(oxetan-3-yl)piperazin-1-yl]benzamide

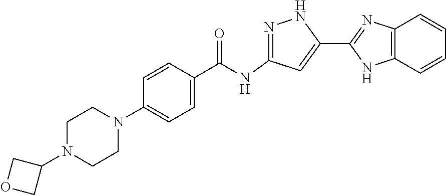 [SMILES: O=C(Nc1cc(-c2nc3ccccc3[nH]2)[nH]n1)c1ccc(N2CCN(C3COC3)CC2)cc1]

TFA (1.50 mL) was added to N-[5-(1H-benzimidazol-2-yl)-1-[(4-methoxyphenyl)methyl]pyrazol-3-yl]-4-[4-(oxetan-3-yl)piperazin-1-yl]benzamide (67 mg, 0.119 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide the title compound as a solid in a 5:2 mixture of tautomers (37.8 mg, 72%). ¹H NMR (400 MHz, DMSO) δ 13.37 [12.69] (s, 1H), 12.96 (s, 1H), [10.82] 10.61 (s, 1H), 7.89 (d, J=8.7 Hz, 2H), 7.68-7.40 (m, 2H), 7.35 [6.59] (s, 1H), 7.22-7.08 (m, 2H), 7.03-6.90 (m, 2H), 4.53 (t, J=6.5 Hz, 2H), 4.43 (t, J=6.1 Hz, 2H), 3.40 (p, J=6.4 Hz, 1H), 3.30 (dd, J=13.9, 3.4 Hz, 4H), 2.39-2.32 (m, 4H). m/z (ES⁺), [M+H]⁺ 444.6. HPLC (A05) t_{R}=1.93 min.

### EXAMPLE B14

### Preparation of Compounds A-95, A-125, A-126, A-164, A-165, A-166, A-167, A-175, A-185, A-202, and A-223

### Representative Synthetic Scheme for the Syntheses of the Title Compounds

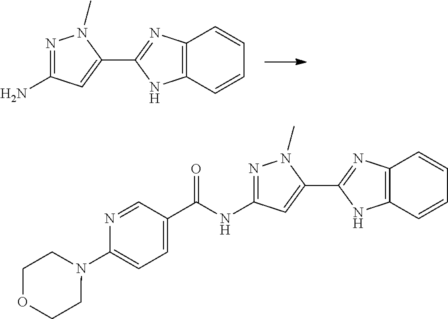 [SMILES: Cn1nc(N)cc1-c1nc2ccccc2[nH]1 ; Cn1nc(NC(=O)c2ccc(N3CCOCC3)nc2)cc1-c1nc2ccccc2[nH]1]

### Preparation of Compound A-95 (N-[5-(1H-benzimidazol-2-yl)-1-methyl-pyrazol-3-yl]-4-morpholino-benzamide)

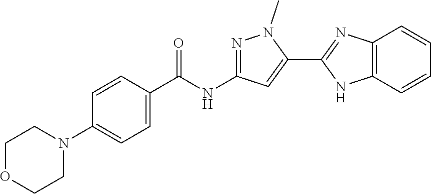 [SMILES: Cn1nc(NC(=O)c2ccc(N3CCOCC3)cc2)cc1-c1nc2ccccc2[nH]1]

DIEA (143 μL, 0.821 mmol) was added to a solution of HATU (98.1 mg, 0.258 mmol) and 4-morpholinobenzoic acid (K-16) (51 mg, 0.246 mmol) in DMF (1 mL) at 0° C. The mixture was stirred at 0° C. for 10 min. 5-(1H-Benzimidazol-2-yl)-1-methyl-pyrazol-3-amine (J-11) (50 mg, 0.234 mmol) was added. The mixture was warmed to 23° C. and stirred for 18 h. Water (10 mL) was added, and the mixture was stirred at 23° C. for 5 min. The mixture was filtered, and the solid was purified by silica gel chromatography (25 g cartridge) eluting with hexanes and EtOAc (0-100%) to provide the title compound as a solid (45.2 mg, 48%). ¹H NMR (500 MHz, DMSO) δ 13.06 (s, 1H), 10.75 (s, 1H), 8.03-7.95 (m, 2H), 7.76-7.70 (m, 1H), 7.57-7.52 (m, 1H), 7.49 (s, 1H), 7.30-7.26 (m, 1H), 7.26-7.20 (m, 1H), 7.04-6.97 (m, 2H), 4.31 (s, 3H), 3.80-3.72 (m, 4H), 3.28-3.22 (m, 4H). m/z (ES+), [M+H]⁺ 403.4. HPLC (A05) t_{R}=2.18 min.

### Preparation of Compound A-129 (3-chloro-N-[5-(7-fluoro-1H-benzimidazol-2-yl)-1-methyl-pyrazol-3-yl]-4-methoxy-benzamide)

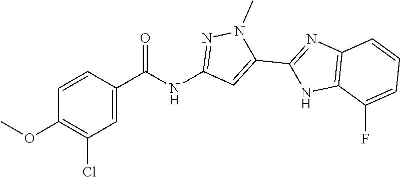 [SMILES: COc1ccc(C(=O)Nc2cc(-c3nc4cccc(F)c4[nH]3)n(C)n2)cc1Cl]

DIEA (132 μL, 0.757 mmol) was added to a solution of HATU (90.4 mg, 0.238 mmol) and 3-chloro-4-methoxy-benzoic acid (42.4 mg, 0.227 mmol) in DMF (1 mL) at 0° C. The mixture was stirred at 0° C. for 10 min. 5-(7-Fluoro-1H-benzimidazol-2-yl)-1-methyl-pyrazol-3-amine (J-13) (50 mg, 0.216 mmol) was added. The mixture was warmed to 23° C. and stirred for 2 h. The mixture was poured into water (20 mL) and stirred at 23° C. for 5 min. The mixture was filtered, and the solid was dissolved in EtOAc (20 mL). The organic phase was dried (MgSO₄), filtered, and concentrated. The residue was purified by silica gel chromatography (16 g cartridge) eluting with hexanes and EtOAc (0-100%). The product was dissolved in DMSO (0.500 mL). Water (10 mL) was added, and the mixture was stirred at 23° C. for 5 min. The mixture was filtered, and the solid was dried under high vacuum to provide the title compound as a solid in a 4:1 mixture of benzimidazole tautomers (52.4 mg, 61%). ¹H NMR (400 MHz, DMSO) δ 13.40 [13.59] (s, 1H), 11.09 [11.06] (s, 1H), 8.17 (d, J=2.2 Hz, 1H), 8.09 (dd, J=8.7, 2.2 Hz, 1H), 7.52 [7.64] (s, 1H), 7.39 [7.58] (d, J=8.2 Hz, 1H), 7.32-7.01 (m, 3H), 4.32 [4.31] (s, 3H), 3.96 (s, 3H). 19F NMR (376 MHz, DMSO) δ −128.70 (ddd, J=11.1, 4.6, 1.6 Hz) [−129.47 (dd, J=10.7, 5.1 Hz)]. m/z (ES⁺), [M+H]⁺ 400.5. HPLC (A05) t_{R}=2.41 min.

### Preparation of Compound A-126 (3-chloro-N-[5-(5-fluoro-1H-benzimidazol-2-yl)-1-methyl-pyrazol-3-yl]-4-methoxy-benzamide)

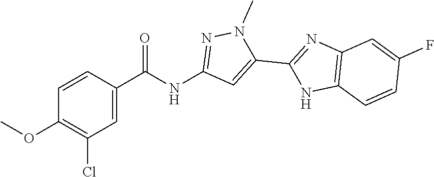 [SMILES: COc1ccc(C(=O)Nc2cc(-c3nc4cc(F)ccc4[nH]3)n(C)n2)cc1Cl]

DIEA (132 μL, 0.757 mmol) was added to a solution of HATU (90.4 mg, 0.238 mmol) and 3-chloro-4-methoxy-benzoic acid (42.4 mg, 0.227 mmol) in DMF (1 mL) at 0° C. The mixture was stirred at 0° C. for 10 min. 5-(5-Fluoro-1H-benzimidazol-2-yl)-1-methyl-pyrazol-3-amine (J-12) (50 mg, 0.216 mmol) was added. The mixture was warmed to 23° C. and stirred for 2 h. The mixture was poured into water (20 mL) and stirred at 23° C. for 5 min. The mixture was filtered, and the solid was dissolved in EtOAc (20 mL). The organic phase was dried (MgSO₄), filtered, and concentrated. The residue was purified by silica gel chromatography (16 g cartridge) eluting with hexanes and EtOAc (0-100%). The product was dissolved in DMSO (0.500 mL). Water (10 mL) was added, and the mixture was stirred at 23° C. for 5 min. The mixture was filtered, and the solid was dried under high vacuum to provide the title compound as a solid in a a 1:1 mixture of benzimidazole tautomers (45.7 mg, 53%). ¹H NMR (400 MHz, DMSO) δ 13.23 [13.22] (s, 1H), 11.07 [11.06] (s, 1H), 8.17 (d, J=2.2 Hz, 1H), 8.08 (dd, J=8.7, 2.2 Hz, 1H), 7.74 [7.54] (dd, J=8.7, 4.9 Hz, 1H), 7.54 [7.33] (dd, J=8.9, 2.4 Hz, 1H), 7.50 [7.49] (s, 1H), 7.29 (d, J=8.8 Hz, 1H), 7.19-7.06 (m, 1H), 4.31 [4.30] (s, 3H), 3.96 (s, 3H). ¹⁹F NMR (376 MHz, DMSO) δ −118.78 [−121.02] (m). m/z (ES⁺), [M+H]⁺ 400.5. HPLC (A05) t_{R}=2.39 min.

### Preparation of Compound A-164 (3-chloro-N-[5-(7-fluoro-1H-benzimidazol-2-yl)-1-methyl-pyrazol-3-yl]-4-(2-methoxyethoxy)benzamide)

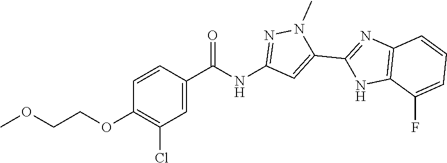 [SMILES: COCCOc1ccc(C(=O)Nc2cc(-c3nc4cccc(F)c4[nH]3)n(C)n2)cc1Cl]

3-Chloro-4-(2-methoxyethoxy)benzoic acid (K-1) (59.8 mg, 0.259 mmol) and HATU (0.0987 g, 0.259 mmol) were dissolved in DMF (1.50 mL) at 0° C. DIEA (0.0888 mL, 0.519 mmol) was added, and the mixture was stirred at 0° C. for 5 min. 5-(7-Fluoro-1H-benzimidazol-2-yl)-1-methyl-pyrazol-3-amine (J-13) (60 mg, 0.259 mmol) was added. The mixture was warmed to 23° C. and stirred for 18 h. The mixture was concentrated. EtOAc (150 mL) was added, and the organic phase was washed with water (3×50 mL) and brine (50 mL), dried (Na₂SO₄), filtered, and concentrated. The residue was purified by silica gel chromatography (12 g cartridge) eluting with DCM and MeOH (0-5%) to provide the title compound as a solid (77.3 mg, 67%). ¹EINMR (400 MHz, DMSO) δ [13.56] 13.38 (s, 1H), 11.07 (s, 1H), 8.16 (d, J=2.2 Hz, 1H), 8.05 (dd, J=8.7, 2.2 Hz, 1H), 7.51 (s, 1H), 7.37 (d, J=8.2 Hz, 1H), 7.29 (d, J=8.8 Hz, 1H), 7.26-7.19 (m, 1H), 7.09-6.99 (m, 1H), 4.32-4.27 (m, 5H), 3.75-3.71 (m, 2H), 3.35 (s, 3H). ¹⁹F NMR (376 MHz, DMSO) δ −128.69 (s). m/z (ES⁺), [M+H]⁺ 444.1. HPLC (A05) t_{R}=2.42 min.

### Preparation of Compound A-165 (3-chloro-N-[5-(7-fluoro-1H-benzimidazol-2-yl)-1-methyl-pyrazol-3-yl]-4-(2-hydroxyethoxy)benzamide)

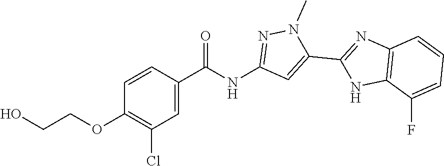 [SMILES: Cn1nc(NC(=O)c2ccc(OCCO)c(Cl)c2)cc1-c1nc2cccc(F)c2[nH]1]

3-Chloro-4-(2-hydroxyethoxy)benzoic acid (K-8) (56.2 mg, 0.259 mmol) and HATU (0.0987 g, 0.259 mmol) were dissolved in DMF (1.50 mL) at 0° C. DIEA (0.0888 mL, 0.519 mmol) was added, and the mixture was stirred at 0° C. for 5 min. 5-(7-Fluoro-1H-benzimidazol-2-yl)-1-methyl-pyrazol-3-amine (J-13) (60 mg, 0.259 mmol) was added. The mixture was warmed to 23° C. and stirred for 18 h. The mixture was concentrated. EtOAc (150 mL) was added, and the organic phase was washed with water (3×50 mL) and brine (50 mL), dried (Na₂SO₄), filtered, and concentrated. The residue was purified by silica gel chromatography (12 g cartridge) eluting with DCM and MeOH (0-10%) to provide the title compound as a solid (69.7 mg, 62%). ¹EINMR (400 MHz, DMSO) δ [13.56] 13.38 (s, 1H), 11.06 (s, 1H), 8.16 (d, J=2.2 Hz, 1H), 8.04 (dd, J=8.7, 2.2 Hz, 1H), 7.51 (s, 1H), 7.37 (d, J=7.5 Hz, 1H), 7.29 (d, J=8.8 Hz, 1H), 7.27-7.20 (m, 1H), 7.10-7.00 (m, 1H), 4.93 (t, J=5.3 Hz, 1H), 4.31 (s, 3H), 4.19 (t, J=5.0 Hz, 2H), 3.78 (dd, J=10.0, 5.3 Hz, 2H). ¹⁹F NMR (376 MHz, DMSO) δ −128.72 (s). m/z (ES⁺), [M+H]⁺ 430.1. HPLC (A05) t_{R}=2.22 min.

### Preparation of Compound A-166 (3-chloro-N-[5-(5-fluoro-1H-benzimidazol-2-yl)-1-methyl-pyrazol-3-yl]-4-(2-methoxyethoxy)benzamide)

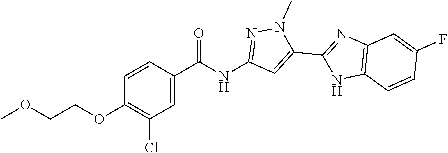 [SMILES: COCCOc1ccc(C(=O)Nc2cc(-c3nc4cc(F)ccc4[nH]3)n(C)n2)cc1Cl]

3-Chloro-4-(2-methoxyethoxy)benzoic acid (K-1) (59.8 mg, 0.259 mmol) and HATU (0.0987 g, 0.259 mmol) were dissolved in DMF (1.50 mL) at 0° C. DIEA (0.0888 mL, 0.519 mmol) was added, and the mixture was stirred at 0° C. for 5 min. 5-(5-Fluoro-1H-benzimidazol-2-yl)-1-methyl-pyrazol-3-amine (J-12) (60 mg, 0.259 mmol) was added. The mixture was warmed to 23° C. and stirred for 18 h. The mixture was concentrated. EtOAc (150 mL) was added, and the organic phase was washed with water (3×50 mL) and brine (50 mL), dried (Na₂SO₄), filtered, and concentrated. The residue was purified by silica gel chromatography (12 g cartridge) eluting with DCM and MeOH (0-5%) to provide the title compound as a solid (76.6 mg, 66%). ¹H NMR (400 MHz, DMSO) δ 13.22 (s, 1H), 11.05 (s, 1H), 8.16 (d, J=2.2 Hz, 1H), 8.04 (dd, J=8.7, 2.2 Hz, 1H), 7.77-7.63 [7.59-7.52] (m, 1H), 7.48 (s, 1H), 7.29 (d, J=8.8 Hz, 1H), 7.11 (t, J=9.5 Hz, 1H), 4.32-4.27 (m, 5H), 3.75-3.70 (m,2H), 3.35 (s, 3H). m/z (ES⁺), [M+H]⁺ 444.1. HPLC (A05) t_{R}=2.40 min.

### Preparation of Compound A-167 (3-chloro-N-[5-(5-fluoro-1H-benzimidazol-2-yl)-1-methyl-pyrazol-3-yl]-4-(2-hydroxyethoxy)benzamide)

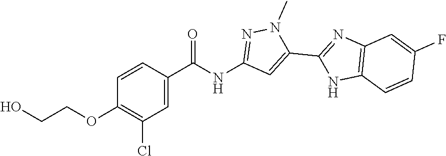 [SMILES: Cn1nc(NC(=O)c2ccc(OCCO)c(Cl)c2)cc1-c1nc2cc(F)ccc2[nH]1]

3-Chloro-4-(2-hydroxyethoxy)benzoic acid (K-8) (56.2 mg, 0.259 mmol) and HATU (0.0987 g, 0.259 mmol) were dissolved in DMF (1.50 mL) at 0° C. DIEA (0.0888 mL, 0.519 mmol) was added, and the mixture was stirred at 0° C. for 5 min. 5-(5-Fluoro-1H-benzimidazol-2-yl)-1-methyl-pyrazol-3-amine (J-12) (60 mg, 0.259 mmol) was added. The mixture was warmed to 23° C. and stirred for 18 h. The mixture was concentrated. EtOAc (150 mL) was added, and the organic phase was washed with water (3×50 mL) and brine (50 mL), dried (Na₂SO₄), filtered, and concentrated. The residue was purified by silica gel chromatography (12 g cartridge) eluting with DCM and MeOH (0-10%). The product was dissolved in EtOAc (150 mL), and the organic phase was washed with sat. NaHCO₃ (75 mL) and water (75 mL) and concentrated to provide the title compound as a solid (64.8 mg, 58%). ¹H NMR (500 MHz, DMSO) δ 13.22 [13.21] (s, 1H), 11.04 [11.04] (s, 1H), 8.16 (d, J=2.3 Hz, 1H), 8.04 (dd, J=8.7, 2.2 Hz, 1H), 7.73 [7.32] (dd, J=8.9, 4.9 Hz, 1H), 7.53 (dt, J=6.8, 2.7 Hz, 1H), 7.48 (d, J=2.2 Hz, 1H), 7.29 (d, J=8.8 Hz, 1H), 7.11 (dddd, J=25.0, 10.0, 8.9, 2.5 Hz, 1H), 4.92 (t, J=5.4 Hz, 1H), 4.29 [4.29] (s, 3H), 4.19 (t, J=5.0 Hz, 2H), 3.78 (dd, J=10.0, 5.3 Hz, 2H). m/z (ES⁺), [M+H]⁺ 430.2. HPLC (A05) t_{R}=2.20 min.

### Preparation of Compound A-175 (N-[5-(1H-benzimidazol-2-yl)-1-(2-methoxyethyl)pyrazol-3-yl]-3-chloro-4-methoxy-benzamide)

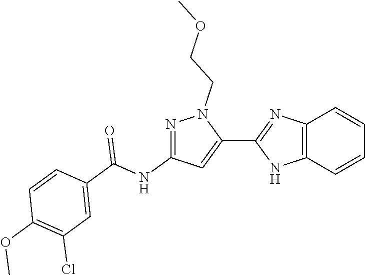 [SMILES: COCCn1nc(NC(=O)c2ccc(OC)c(Cl)c2)cc1-c1nc2ccccc2[nH]1]

DIEA (0.317 mL, 1.82 mmol) was added to a solution of HATU (0.242 g, 0.637 mmol) and 3-chloro-4-methoxy-benzoic acid (0.322 g, 1.73 mmol) in DMF (5 mL) at 0° C. The mixture was stirred at 0° C. for 10 min. 5-(1H-Benzimidazol-2-yl)-1-(2-methoxyethyl)pyrazol-3-amine (J-15) (0.117 g, 0.455 mmol) was added. The mixture was warmed to 23° C. and stirred for 18 h. Water (50 mL) was added, and the aq. phase was extracted with EtOAc (3×20 mL). The combined organic phases were washed with sat. NaHCO₃ (50 mL) and brine (50 mL), dried (MgSO₄), filtered, and concentrated. The residue was purified by silica gel chromatography (7 g cartridge) eluting in hexanes and EtOAc (30-80%) to provide the title compound as a solid (171 mg, 88%). ¹H NMR (400 MHz, DMSO) δ 13.08 (s, 1H), 11.10 (s, 1H), 8.17 (d, J=2.2 Hz, 1H), 8.09 (dd, J=8.7, 2.2 Hz, 1H), 7.71 (s, 1H), 7.53 (s, 1H), 7.50 (s, 1H), 7.28 (d, J=8.8 Hz, 3H), 5.02 (t, J=5.7 Hz, 2H), 3.95 (s, 3H), 3.81 (t, J=5.7 Hz, 2H), 3.21 (s, 3H). m/z (ES⁺), [M−H]− 424.5. HPLC (A05) t_{R}=2.37 min.

### Preparation of Compound A-185 (N-[5-(1H-benzimidazol-2-yl)-1-(2-methoxyethyl)pyrazol-3-yl]-4-(4-methylpiperazin-1-yl)benzamide)

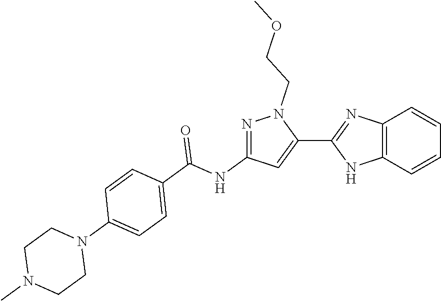 [SMILES: COCCn1nc(NC(=O)c2ccc(N3CCN(C)CC3)cc2)cc1-c1nc2ccccc2[nH]1]

DIEA (0.199 mL, 1.143 mmol) was added to a solution of 4-(4-methylpiperazin-1-yl)benzoic acid hydrochloride (K-15) (81.6 mg, 0.318 mmol) and HATU (134 mg, 0.351 mmol) in DMF (2 mL) at 0° C. The mixture was stirred at 0° C. for 10 min. 5-(1H-Benzimidazol-2-yl)-1-(2-methoxyethyl)pyrazol-3-amine (J-15) (98 mg, 0.381 mmol) was added. The mixture was stirred at 23° C. for 18 h, warmed at 50° C. for 4 h, and heated at 70° C. for 1 h. The mixture was cooled to 23° C., and water (20 mL) was added. The aq. phase was extracted with EtOAc (3×10 mL). The combined organic phases were washed with sat. NaHCO₃ (10 mL) and brine (10 mL), dried (MgSO₄), filtered, and concentrated under reduced pressure. The residue was purified by preparative HPLC (BEH C18 30×150) eluting with water (10 mM (NH₄)(HCO₃)) and MeCN (34-55%) to provide the title compound as a solid (85 mg, 58%). ¹H NMR (500 MHz, DMSO) δ 13.04 (s, 1H), 10.76 (s, 1H), 7.97 (d, J=9.0 Hz, 2H), 7.70 (s, 1H), 7.53 (s, 1H), 7.49 (s, 1H), 7.25 (s, 2H), 6.98 (d, J=9.1 Hz, 2H), 5.00 (t, J=5.7 Hz, 2H), 3.80 (t, J=5.7 Hz, 2H), 3.30-3.27 (m, 4H), 3.21 (s, 3H), 2.47-2.42 (m, 4H), 2.23 (s, 3H). m/z (ES⁻), [M−H]⁻ 458.6. HPLC (A05) t_{R}=1.88 min.

### Preparation of Compound A-202 (N-[5-(1H-benzimidazol-2-yl)-1-methyl-pyrazol-3-yl]-3-chloro-4-(2-hydroxyethoxy)benzamide)

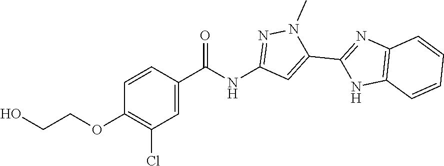 [SMILES: Cn1nc(NC(=O)c2ccc(OCCO)c(Cl)c2)cc1-c1nc2ccccc2[nH]1]

3-Chloro-4-(2-hydroxyethoxy)benzoic acid (K-8) (40.6 mg, 0.188 mmol) and HATU (0.0713 g, 0.188 mmol) were dissolved in DMF (1 mL) at 0° C. DIEA (0.0642 mL, 0.375 mmol) was added, and the mixture was stirred at 0° C. for 5 min. 5-(1H-Benzimidazol-2-yl)-1-methyl-pyrazol-3-amine (J-11) (40 mg, 0.188 mmol) was added. The mixture was warmed to 23° C. and stirred for 18 h. The mixture was concentrated, and the residue was purified by silica gel chromatography (12 g cartridge) eluting with DCM and MeOH (0-10%). The product was dissolved in EtOAc (150 mL), and the organic phase was washed with water (50 mL), sat. NaHCO₃ (2×50 mL), water (50 mL), and brine (50 mL), dried (Na₂SO₄), filtered, and concentrated to provide the title compound as a solid (61.5 mg, 80%). ¹H NMR (400 MHz, DMSO) δ 13.03 (s, 1H), 11.00 (s, 1H), 8.12 (d, J=2.2 Hz, 1H), 8.01 (dd, J=8.7, 2.3 Hz, 1H), 7.68 (d, J=6.7 Hz, 1H), 7.50 (d, J=6.5 Hz, 1H), 7.45 (s, 1H), 7.28-7.15 (m, 3H), 4.90 (t, J=5.3 Hz, 1H), 4.27 (s, 3H), 4.17-4.13 (m, 2H), 3.74 (dd, J=10.0, 5.2 Hz, 2H). m/z (ES⁺), [M+H]⁺ 412.2. HPLC (A05) t_{R}=2.12 min.

### Preparation of Compound A-223 (N-[5-(1H-benzimidazol-2-yl)-1-methyl-pyrazol-3-yl]-4-(2-hydroxyethoxy)-3-(trifluoromethyl)benzamide)

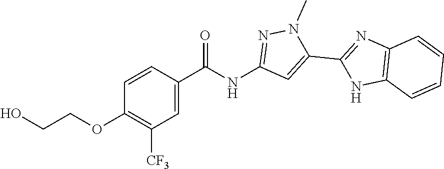 [SMILES: Cn1nc(NC(=O)c2ccc(OCCO)c(C(F)(F)F)c2)cc1-c1nc2ccccc2[nH]1]

4-(2-Hydroxyethoxy)-3-(trifluoromethyl)benzoic acid (K-12) (70 mg, 0.280 mmol) and HATU (0.106 g, 0.280 mmol) were dissolved in DMF (2 mL) at 0° C. DIEA (0.0958 mL, 0.560 mmol) was added, and the mixture was stirred at 0° C. for 5 min. 5-(1H-Benzimidazol-2-yl)-1-methyl-pyrazol-3-amine (J-11) (59.7 mg, 0.280 mmol) was added. The mixture was warmed to 23° C. and stirred for 18 h. The mixture was concentrated, and the residue was purified by silica gel chromatography (12 g cartridge) eluting with DCM and MeOH (0-10%). The product was dissolved in DMSO. Water (10 mL) was added. The mixture was filtered, and the solid was purified by silica gel chromatography (12 g cartridge) eluting with DCM and MeCN (0-100%) and by preparative HPLC (BEH C18 30×150) eluting with water (10 mM (NH₄)(HCO₃)) and MeCN (32-42%) to provide the title compound as a solid (11.8 mg, 9%). ¹H NMR (400 MHz, DMSO) δ 13.20-12.87 (m, 1H), 11,20 (s, 1H), 8.37-8.30 (m, 2H), 7.76-7.65 (m, 1H), 7.61-7.51 (m, 1H), 7.49 (s, 1H), 7.42 (d, J=8.6 Hz, 1H), 7.26 (s, 2H), 4.89 (t, J=5.3 Hz, 1H), 4.31 (s, 3H), 4.24 (t, J=5.1 Hz, 2H), 3.76 (dd, J=10.2, 5.2 Hz, 2H). ¹⁹F NMR (376 MHz, DMSO) δ −61.01 (s). m/z (ES⁺), [M+H]⁺ 446.4. HPLC (A05) t_{R}=2.23 min.

### EXAMPLE B15

### Preparation of Compounds A-96, A-97, A-128, A-133, A-134, A-135, A-136, A-137, A-139, A-148, A-149, A-150, A-153, A-154, A-157, A-158, A-160, A-168, A-173, A-176, A-178, A-182, A-187, A-189, A-190, A-191, A-203, A-204, A-205, A-206, A-207, A-208, A-209, A-210, A-211, A-212, A-213, A-214, A-215, A-216, A-217, A-219, A-221, A-222, A-224, A-225, A-226, and A-227

### Representative Synthetic Scheme for the Syntheses of the Title Compounds.

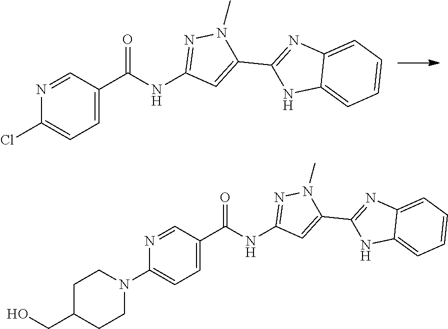 [SMILES: Cn1nc(NC(=O)c2ccc(Cl)nc2)cc1-c1nc2ccccc2[nH]1 ; Cn1nc(NC(=O)c2ccc(N3CCC(CO)CC3)nc2)cc1-c1nc2ccccc2[nH]1]

### Preparation of Compound A-96 (N-[5-(1H-benzimidazol-2-yl)-1-methyl-pyrazol-3-yl]-6-[4-(hydroxy-methyl)-1-piperidyl]pyridine-3-carboxamide)

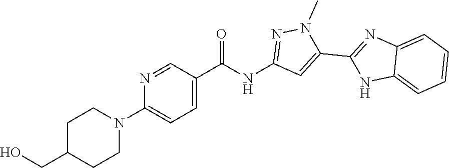 [SMILES: Cn1nc(NC(=O)c2ccc(N3CCC(CO)CC3)nc2)cc1-c1nc2ccccc2[nH]1]

N-[5-(1H-Benzimidazol-2-yl)-1-methyl-pyrazol-3-yl]-6-chloro-pyridine-3-carboxamide (L-11) (50 mg, 0.142 mmol), 4-piperidylmethanol (24.5 mg, 0.213 mmol), and DIEA (49.4 μL, 0.283 mmol) were dissolved in DMSO (0.500 mL). The mixture was stirred at 100° C. for 18 h. Water (25 mL) was added. The mixture was filtered, and the solid was purified by silica gel chromatography eluting with hexanes and EtOAc (0-100%) to provide the title compound as a solid (36.7 mg, 60%). ¹H NMR (500 MHz, DMSO) δ 13.06 (s, 1H), 10.79 (s, 1H), 8.79 (d, J=2.4 Hz, 1H), 8.13 (dd, J=9.1, 2.5 Hz, 1H), 7.79-7.66 (m, 1H), 7.61-7.51 (m, 1H), 7.48 (s, 1H), 7.31-7.19 (m, 2H), 6.85 (d, J=9.1 Hz, 1H), 4.59 (t, J=5.3 Hz, 1H), 4.43 (d, J=13.8 Hz, 1H), 4.31 (s, 3H), 4.35-4.25 (m, 1H), 3.41-3.27 (m, 2H), 3.01-2.91 (m, 1H), 2.72 (dd, J=13.0, 10.5 Hz, 1H), 1.82-1.74 (m, 1H), 1.74-1.67 (m, 1H), 1.67-1.55 (m, 1H), 1.49-1.38 (m, 1H), 1.31-1.19 (m, 1H). m/z (ES⁺), [M+H]⁺ 432.4. HPLC (A05) t_{R}=2.09 min.

### Preparation of Compound A-97 (N-[5-(1H-benzimidazol-2-yl)-1-methyl-pyrazol-3-yl]-6-(3-methoxyazetidin-1-yl)pyridine-3-carboxamide).

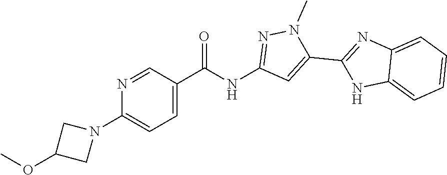 [SMILES: COC1CN(c2ccc(C(=O)Nc3cc(-c4nc5ccccc5[nH]4)n(C)n3)cn2)C1]

N-[5-(1H-Benzimidazol-2-yl)-1-methyl-pyrazol-3-yl]-6-chloro-pyridine-3-carboxamide (L-11) (50 mg, 0.142 mmol), 3-methoxyazetidine hydrochloride (26.3 mg, 0.213 mmol) and DIEA (98.7 μL, 0.567 mmol) were dissolved in DMSO (0.500 mL). The mixture was stirred at 100° C. for 18 h. Water (25 mL) was added. The mixture was filtered, and the solid was purified by silica gel chromatography eluting with hexanes and EtOAc (0-100%) to provide the title compound as a solid (31.2 mg, 55%). ¹H NMR (400 MHz, DMSO) δ 13.07 (s, 1H), 10.84 (s, 1H), 8.78 (d, J=1.9 Hz, 1H), 8.15 (dd, J=8.8, 2.3 Hz, 1H), 7.79-7.63 (m, 1H), 7.63-7.51 (m, 1H), 7.48 (s, 1H), 7.34-7.15 (m, 2H), 6.44 (d, J=8.8 Hz, 1H), 4.41-4.33 (m, 1H), 4.31 (s, 3H), 4.24 (dd, J=8.9, 6.5 Hz, 2H), 3.86 (dd, J=9.5, 3.5 Hz, 2H), 3.28 (s, 3H). m/z (ES⁺), [M+H]⁺ 404.6. HPLC (A05) t_{R}=2.06 min.

### Preparation of Compound A-128 (N-[5-(5-fluoro-1H-benzimidazol-2-yl)-1-methyl-pyrazol-3-yl]-6-(3-methoxyazetidin-1-yl)pyridine-3-carboxamide)

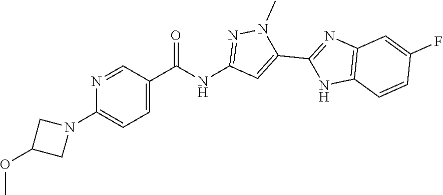 [SMILES: COC1CN(c2ccc(C(=O)Nc3cc(-c4nc5cc(F)ccc5[nH]4)n(C)n3)cn2)C1]

6-Chloro-N-[5-(5-fluoro-1H-benzimidazol-2-yl)-1-methyl-pyrazol-3-yl]pyridine-3-carboxamide (L-12) (40 mg, 0.108 mmol), 3-methoxyazetidine hydrochloride (20 mg, 0.162 mmol), and DIEA (75.2 μL, 0.432 mmol) were dissolved in DMSO (0.500 mL). The mixture was stirred at 100° C. for 18 h. Water (10 mL) was added, and the mixture was stirred at 23° C. for 5 min. The solid was filtered, dried in a vacuum over at 45° C. for 2 h, and purified by silica gel chromatography (7 g cartridge) eluting with DCM and MeOH (0-20%). The product was dissolved in DMSO (0.500 mL). Water (10 mL) was added, and the mixture was stirred at 23° C. for 5 min. The mixture was filtered, and the solid was dried under high vacuum to provide the title compound as a solid in a 1:1 mixture of benzimidazole tautomers (21 mg, 46%). ¹H NMR (500 MHz, DMSO) δ 13.20 (s, 1H), 10.84 (s, 1H), 8.78 (d, J=2.0 Hz, 1H), 8.14 (dd, J=8.7, 2.2 Hz, 1H), 7.80-7.63 [7.63-7.51] (m, 1H), 7.63-7.51 [7.39-7.27] (m, 1H), 7.47 (s, 1H), 7.18-7.04 (m, 1H), 6.44 (d, J=8.6 Hz, 1H), 4.45-4.33 (m, 1H), 4.29 (s, 3H), 4.27-4.18 (m, 2H), 3.91-3.78 (m, 2H), 3.28 (s, 3H). ¹⁹F NMR (471 MHz, DMSO) δ −118.78 [−121.02]. m/z (ES⁺), [M+H]⁺ 422.6. HPLC (A05) t_{R}=2.13 min.

### Preparation of Compound A-133 (6-[4-(dimethylamino)-1-piperidyl]-N-[5-(5-fluoro-1H-benzimidazol-2-yl)-1-methyl-pyrazol-3-yl]pyridine-3-carboxamide)

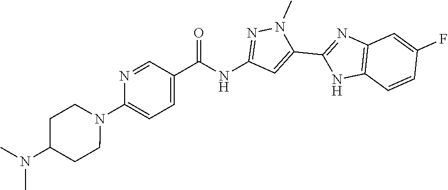 [SMILES: CN(C)C1CCN(c2ccc(C(=O)Nc3cc(-c4nc5cc(F)ccc5[nH]4)n(C)n3)cn2)CC1]

6-Chloro-N-[5-(5-fluoro-1H-benzimidazol-2-yl)-1-methyl-pyrazol-3-yl]pyridine-3-carboxamide (L-12) (40 mg, 0.108 mmol), N,N-dimethylpiperidin-4-amine (20.7 mg, 0.162 mmol), and DIEA (37.6 μL, 0.216 mmol) were dissolved in DMSO (0.500 mL). The mixture was stirred at 100° C. for 18 h. Water (10 mL) was added, and the mixture was stirred at 23° C. for 5 min. The mixture was filtered, and the solid was dried in a vacuum oven at 45° C. for 2 h and purified by silica gel chromatography (7 g cartridge) eluting with DCM and MeOH (0-20%)+1% NEt₃. The product was dissolved in DMSO (0.500 mL). Water (10 mL) was added, and the mixture was stirred at 23° C. for 5 min. The mixture was filtered and dried under high vacuum to provide the title compound as a solid in a 1:1 mixture of benzimidazole tautomers (12.3 mg, 25%). ¹H NMR (400 MHz, DMSO) δ 13.21 (s, 1H), 10.82 (s, 1H), 8.79 (d, J=2.3 Hz, 1H), 8.13 (dd, J=9.0, 2.3 Hz, 1H), 7.78-7.68 [7.60-7.50] (m, 1H), 7.60-7.50 [7.38-7.29] (m, 1H), 7.48 (s, 1H), 7.18-7.05 (m, 1H), 6.90 (d, J=9.0 Hz, 1H), 4.52-4.39 (m, 2H), 4.29 (s, 3H), 2.99-2.85 (m, 2H), 2.44-2.31 (m, 1H), 2.19 (s, 6H), 1.87-1.78 (m, 2H), 1.43-1.27 (m, 2H). ¹⁹F NMR (376 MHz, DMSO) δ −118.82 [−121.07]. m/z (ES⁺), [M+H]⁺ 463.6. HPLC (A05) t_{R}=1.89 min.

### Preparation of Compound A-134 (N-[5-(4-fluoro-1H-benzimidazol-2-A-1-methyl-pyrazol-3-yl]-6-(4-hydroxy-1-piperidyl)pyridine-3-carboxamide)

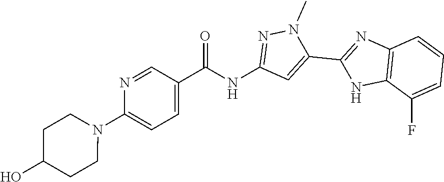 [SMILES: Cn1nc(NC(=O)c2ccc(N3CCC(O)CC3)nc2)cc1-c1nc2cccc(F)c2[nH]1]

6-Chloro-N-[5-(4-fluoro-1H-benzimidazol-2-yl)-1-methyl-pyrazol-3-yl]pyridine-3-carboxamide (L-13) (45 mg, 0.121 mmol), piperidin-4-ol (0.0184 g, 0.182 mmol), and DIEA (0.0416 mL, 0.243 mmol) were dissolved in DMSO (1 mL). The mixture was stirred at 100° C. for 18 h. EtOAc (100 mL) was added. The organic phase was washed with water (3×50 mL) and brine (50 mL), dried (Na₂SO₄), filtered, and concentrated. The residue was purified by silica gel chromatography (12 g cartridge) eluting with DCM and MeOH (0-20%). The product was dissolved in DMSO (0.500 mL). Water (10 mL) was added, the mixture was filtered, and the solid was dried to provide the title compound as a solid (31.3 mg, 59%). ¹H NMR (400 MHz, DMSO) δ [13.54] 13.36 (s, 1H), 10.82 (s, 1H), 8.78 (d, J=2.4 Hz, 1H), 8.12 (dd, J=9.1, 2.5 Hz, 1H), 7.49 (s, 1H), 7.37 (d, J=7.7 Hz, 1H), 7.24 (dd, J=12.1, 6.8 Hz, 1H), 7.10-6.98 (m, 1H), 6.89 (d, J=9.1 Hz, 1H), 4.73 (d, J=4.3 Hz, 1H), 4.30 (s, 3H), 4.11 (dt, J=13.2, 4.2 Hz, 2H), 3.79-3.70 (m, 1H), 3.24 (ddd, J=13.1, 9.9, 3.1 Hz, 2H), 1.84-1.74 (m, 2H), 1.41-1.30 (m, 2H). ¹⁹F NMR (376 MHz, DMSO) δ −128.71 (s). m/z (ES⁺), [M+H]⁺ 436.1. HPLC (A05) t_{R}=2.07 min.

### Preparation of Compound A-135 (N-[5-(4-fluoro-1H-benzimidazol-2-yl)-1-methyl-pyrazol-3-yl]-6-morpholino-pyridine-3-carboxamide)

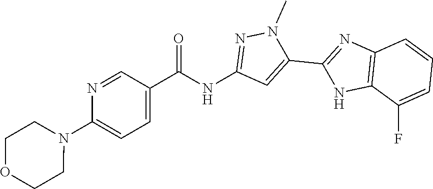 [SMILES: Cn1nc(NC(=O)c2ccc(N3CCOCC3)nc2)cc1-c1nc2cccc(F)c2[nH]1]

6-Chloro-N-[5-(4-fluoro-1H-benzimidazol-2-yl)-1-methyl-pyrazol-3-yl]pyridine-3-carboxamide (L-13) (45 mg, 0.121 mmol), morpholine (0.0157 mL, 0.182 mmol), and DIEA (0.0416 mL, 0.243 mmol) were dissolved in DMSO (1 mL). The mixture was stirred at 100° C. for 18 h. EtOAc (100 mL) was added. The organic phase was washed with water (3×50 mL) and brine (50 mL), dried (Na₂SO₄), filtered, and concentrated. The residue was purified by silica gel chromatography (12 g cartridge) eluting with DCM and MeOH (0-20%). The residue was dissolved in DMSO (0.500 mL). Water (10 mL) was added, the mixture was filtered, and the solid was dried to provide the title compound as a solid (25 mg, 49%). ¹H NMR (400 MHz, DMSO) δ 13.37 (s, 1H), 10.89 (s, 1H), 8.82 (d, J=2.2 Hz, 1H), 8.18 (dd, J=9.1, 2.5 Hz, 1H), 7.50 (s, 1H), 7.37 (d, J=8.0 Hz, 1H), 7.25 (t, J=10.1 Hz, 1H), 7.08-7.01 (m, 1H), 6.90 (d, J=9.1 Hz, 1H), 4.30 (s, 3H), 3.73-3.67 (m, 4H), 3.62-3.56 (m, 4H). ¹⁹F NMR (376 MHz, DMSO) δ −128.74 (s). m/z (ES⁺), [M+H]⁺ 422.0. HPLC (A05) t_{R}=2.18 min.

### Preparation of Compound A-136 (N-[5-(1H-benzimidazol-2-yl)-1-[(4-methoxyphenyl)methyl]pyrazol-3-yl]-4-(2-hydroxyethoxy)-3-methoxy-benzamide)

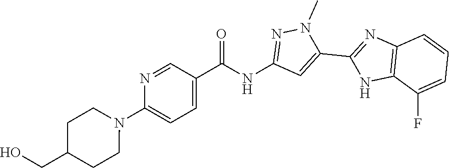 [SMILES: Cn1nc(NC(=O)c2ccc(N3CCC(CO)CC3)nc2)cc1-c1nc2cccc(F)c2[nH]1]

6-Chloro-N-[5-(4-fluoro-1H-benzimidazol-2-yl)-1-methyl-pyrazol-3-yl]pyridine-3-carboxamide (L-13) (45 mg, 0.121 mmol), 4-piperidylmethanol (0.0210 g, 0.182 mmol), and DIEA (0.0416 mL, 0.243 mmol) were dissolved in DMSO (1 mL). The mixture was stirred at 100° C. for 18 h. EtOAc (100 mL) was added. The organic phase was washed with water (3×50 mL) and brine (50 mL), dried (Na₂SO₄), filtered, and concentrated. The residue was purified by silica gel chromatography (12 g cartridge) eluting with DCM and MeOH (0-20%). The product was dissolved in DMSO (0.500 mL). Water (10 mL) was added, the mixture was filtered, and the solid was dried to provide the title compound as a solid in a 4:1 mixture of tautomers (40.3 mg, 74%). ¹H NMR (400 MHz, DMSO) δ [13.55] 13.36 (s, 1H), 10.82 [10.79] (s, 1H), 8.78 (d, J=2.3 Hz, 1H), 8.12 (dd, J=9.1, 2.5 Hz, 1H), 7.50 (s, 1H), 7.37 (d, J=7.9 Hz, 1H), 7.28-7.21 (m, 1H), 7.09-7.00 (m, 1H), 6.87 (d, J=9.1 Hz, 1H), 4.52-4.41 (m, 3H), 4.30 (s, 3H), 3.27 (d, J=6.3 Hz, 2H), 2.89 (td, J=12.9, 2.3 Hz, 2H), 1.78-1.61 (m, 3H), 1.17-1.03 (m, 2H). ¹⁹F NMR (376 MHz, DMSO) δ −128.70 (s). m/z (ES⁺), [M+H]⁺ 450.1. HPLC (A05) t_{R}=2.12 min.

### Preparation of Compound A-135 (N-[5-(4-fluoro-1H-benzimidazol-2-yl)-1-methyl-pyrazol-3-yl]-6-(3-methoxyazetidin-1-yl)pyridine-3-carboxamide

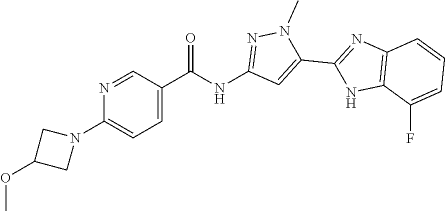 [SMILES: COC1CN(c2ccc(C(=O)Nc3cc(-c4nc5cccc(F)c5[nH]4)n(C)n3)cn2)C1]

6-Chloro-N-[5-(4-fluoro-1H-benzimidazol-2-yl)-1-methyl-pyrazol-3-yl]pyridine-3-carboxamide (L-13) (45 mg, 0.121 mmol), 3-methoxyazetidine hydrochloride (0.0225 g, 0.182 mmol), and DIEA (0.0623 mL, 0.364 mmol) were dissolved in DMSO (1 mL). The mixture was stirred at 100° C. for 18 h. EtOAc (100 mL) was added. The organic phase was washed with water (3×50 mL) and brine (50 mL), dried (Na₂SO₄), filtered, and concentrated. The residue was purified by silica gel chromatography (12 g cartridge) eluting with DCM and MeOH (0-20%). The product was dissolved in DMSO (0.500 mL). Water (10 mL) was added, the mixture was filtered, and the solid was dried to provide the title compound as a solid in a 4:1 mixture of tautomers (30.4 mg, 59%). ¹H NMR (400 MHz, DMSO) δ [13.51] 13.32 (s, 1H), 10.81 [10.78] (s, 1H), 8.72 (dd, J=2.4, 0.7 Hz, 1H), 8.08 (dd, J=8.8, 2.4 Hz, 1H), 7.44 (s, 1H), 7.32 (d, J=7.3 Hz, 1H), 7.22-7.14 (m, 1H), 7.00 (dd, J=11.1, 8.0 Hz, 1H), 6.38 (d, J=9.3 Hz, 1H), 4.34-4.27 (m, 1H), 4.25 [4.24] (s, 3H), 4.21-4.15 (m, 2H), 3.80 (dd, J=10.4, 3.9 Hz, 2H), 3.22 (s, 3H). ¹⁹F NMR (376 MHz, DMSO) δ −128.73 (s). m/z (ES⁺), [M+H]⁺ 422.0. HPLC (A05) t_{R}=2.14 min.

### Preparation of Compound A-139 (6-[4-(dimethylamino)-1-piperidyl]-N-[5-(4-fluoro-1H-benzimidazol-2-yl)-1-methyl-pyrazol-3-yl]pyridine-3-carboxamide)

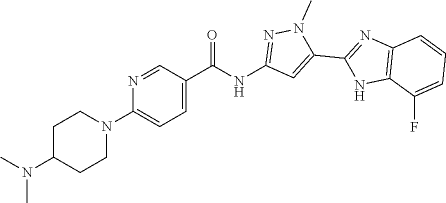 [SMILES: CN(C)C1CCN(c2ccc(C(=O)Nc3cc(-c4nc5cccc(F)c5[nH]4)n(C)n3)cn2)CC1]

6-Chloro-N-[5-(4-fluoro-1H-benzimidazol-2-yl)-1-methyl-pyrazol-3-yl]pyridine-3-carboxamide (L-13) (45 mg, 0.121 mmol), N,N-dimethylpiperidin-4-amine (0.0233 g, 0.182 mmol), and DIEA (0.0416 mL, 0.243 mmol) were dissolved in DMSO (1 mL). The mixture was stirred at 100° C. for 18 h. EtOAc (100 mL) was added. The organic phase was washed with water (3×50 mL) and brine (50 mL), dried (Na₂SO₄), filtered, and concentrated. The residue was first purified by silica gel chromatography (12 g cartridge) eluting with DCM and MeOH (0-20%)+1% NEt₃. The product was then purified by preparative HPLC (BEH C18 30×150) eluting with water (10 mM (NH₄)(HCO₃)) and MeCN (41-51%) to provide the title compound as a solid (21 mg, 37%). ¹H NMR (400 MHz, DMSO) δ 13.36 (s, 1H), 10.83 (s, 1H), 8.78 (d, J=2.3 Hz, 1H), 8.13 (dd, J=9.1, 2.5 Hz, 1H), 7.50 (s, 1H), 7.37 (d, J=8.3 Hz, 1H), 7.25 (dd, J=12.5, 7.7 Hz, 1H), 7.04 (dd, J=16.8, 8.3 Hz, 1H), 6.89 (d, J=9.3 Hz, 1H), 4.43 (d, J=13.0 Hz, 2H), 4.30 (s, 3H), 2.97-2.87 (m, 2H), 2.42-2.34 (m, 1H), 2.18 (s, 6H), 1.83 (d, J=10.7 Hz, 2H), 1.33 (ddd, J=24.6, 12.5, 4.0 Hz, 2H). ¹⁹F NMR (376 MHz, DMSO) δ −128.73 (s). m/z (ES⁺), [M+H]⁺ 463.2. HPLC (A05) t_{R}=1.88 min.

### Preparation of Compound A-148 (N-[5-(5-fluoro-1H-benzimidazol-2-yl)-1-methyl-pyrazol-3-yl]-6-morpholino-pyridine-3-carboxamide)

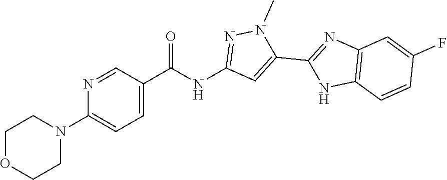 [SMILES: Cn1nc(NC(=O)c2ccc(N3CCOCC3)nc2)cc1-c1nc2cc(F)ccc2[nH]1]

6-Chloro-N-[5-(5-fluoro-1H-benzimidazol-2-yl)-1-methyl-pyrazol-3-yl]pyridine-3-carboxamide (L-12) (40 mg, 0.108 mmol) was dissolved in DMSO (1 mL). DIEA (37.6 μL, 0.216 mmol) and morpholine (14.0 μL, 0.162 mmol) were added. The mixture was heated at 100° C. for 18 h. Water (15 mL) was added, and the mixture was stirred at 23° C. for 5 min. The mixture was filtered, and the solid was purified by silica gel chromatography (12 g cartridge) eluting with DCM and MeOH (0-10%) to provide the title compound as a solid (29.2 mg, 64%). ¹H NMR (500 MHz, DMSO) δ 13.20 (s, 1H), 10.87 (s, 1H), 8.83 (d, J=2.5 Hz, 1H), 8.19 (dd, J=9.0, 2.5 Hz, 1H), 7.83-7.53 (m, 1H), 7.48 (s, 1H), 7.46-7.35 (m, 1H), 7.18-7.04 (m, 1H), 6.91 (d, J=9.1 Hz, 1H), 4.29 (s, 3H), 3.71 (dd, J=5.8, 4.0 Hz, 4H), 3.61 (dd, J=5.7, 4.1 Hz, 4H). ¹⁹F NMR (471 MHz, DMSO) δ −119.01 [−120.80]. m/z (ES⁺), [M+H]⁺ 422.6. HPLC (A05) t_{R}=2.17 min.

### Preparation of Compound A-149 (N-[5-(5-fluoro-1H-benzimidazol-2-A-1-methyl-pyrazol-3-yl]-6-[4-(2-methoxyethyl)piperazin-1-yl]pyridine-3-carboxamide)

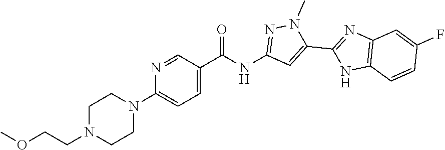 [SMILES: COCCN1CCN(c2ccc(C(=O)Nc3cc(-c4nc5cc(F)ccc5[nH]4)n(C)n3)cn2)CC1]

6-Chloro-N-[5-(5-fluoro-1H-benzimidazol-2-yl)-1-methyl-pyrazol-3-yl]pyridine-3-carboxamide (L-12) (40 mg, 0.108 mmol) was dissolved in DMSO (1 mL). DIEA (37.6 μL, 0.216 mmol) and 1-(2-methoxyethyl)piperazine (24.1 μL, 0.162 mmol) were added. The mixture was heated at 100° C. for 18 h. Water (15 mL) was added, and the mixture was stirred at 23° C. for 5 min. The mixture was filtered, and the solid was purified by silica gel chromatography (12 g cartridge) eluting with DCM and MeOH (0-10%) to provide the title compound as a solid (24.9 mg, 48%). ¹H NMR (400 MHz, CD3OD) δ 8.74 (dd, J=2.6, 0.7 Hz, 1H), 8.09 (dd, J=9.1, 2.5 Hz, 1H), 7.79-7.22 (m, 2H), 7.27 (s, 1H), 7.14-7.03 (m, 1H), 6.87 (d, J=9.1 Hz, 1H), 4.25 (s, 3H), 3.76-3.67 (m, 4H), 3.59 (t, J=5.5 Hz, 2H), 3.37 (s, 3H), 2.79-2.55 (m, 6H). ¹H NMR (500 MHz, DMSO) δ 13.20 (s, 1H), 10.83 (s, 1H), 8.80 (d, J=2.6 Hz, 1H), 8.15 (dd, J=9.0, 2.5 Hz, 1H), 7.84-7.52 (m, 1H), 7.47 (s, 1H), 7.54-7.31 (m, 1H), 7.11 (t, J=9.2 Hz, 1H), 6.89 (d, J=9.1 Hz, 1H), 4.29 (s, 3H), 3.63 (t, J=5.1 Hz, 4H), 3.49 (t, J=5.8 Hz, 2H), 3.26 (s, 3H), 2.60-2.49 (m, 6H). ¹⁹F NMR (471 MHz, DMSO) δ −119.12 [−121.25]. m/z (ES⁺), [M+H]⁺ 479.6. HPLC (A05) t_{R}=1.91 min.

### Preparation of Compound A-150 (N-[5-(5-fluoro-1H-benzimidazol-2-A-1-methyl-pyrazol-3-yl]-6-[4-(2-hydroxyethyl)piperazin-1-yl]pyridine-3-carboxamide)

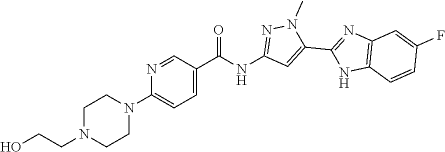 [SMILES: Cn1nc(NC(=O)c2ccc(N3CCN(CCO)CC3)nc2)cc1-c1nc2cc(F)ccc2[nH]1]

6-Chloro-N-[5-(5-fluoro-1H-benzimidazol-2-yl)-1-methyl-pyrazol-3-yl]pyridine-3-carboxamide (L-12) (40 mg, 0.108 mmol) was dissolved in DMSO (1 mL). DIEA (37.6 μL, 0.216 mmol) and 2-piperazin-1-ylethanol (19.9 μL, 0.162 mmol) were added. The mixture was heated at 100° C. for 18 h. Water (15 mL) was added, and the mixture was stirred at 23° C. for 5 min. The mixture was filtered, and the solid was purified by silica gel chromatography (12 g cartridge) eluting with DCM and MeOH (0-10%) to provide the title compound as a solid (21.3 mg, 43%). ¹H NMR (400 MHz, CD3OD) δ 8.76 (dd, J=2.5, 0.7 Hz, 1H), 8.11 (dd, J=9.0, 2.6 Hz, 1H), 7.80-7.26 (m, 2H), 7.29 (s, 1H), 7.11 (t, J=9.2 Hz, 1H), 7.00-6.82 (m, 1H), 4.27 (s, 3H), 3.86-3.62 (m, 6H), 2.69-2.65 (m, 4H), 2.62 (t, J=5.9 Hz, 2H). ¹H NMR (500 MHz, DMSO) δ 13.20 (s, 1H), 10.83 (s, 1H), 8.80 (d, J=2.6 Hz, 1H), 8.15 (dd, J=9.1, 2.5 Hz, 1H), 7.73-7.53 (m, 1H), 7.53-7.32 (m, 1H), 7.47 (s, 1H), 7.22-7.04 (m, 1H), 6.89 (d, J=9.1 Hz, 1H), 4.44 (t, J=5.4 Hz, 1H), 4.29 (s, 3H), 4.08 (q, J=5.3 Hz, 1H), 3.63 (t, J=5.1 Hz, 4H), 3.55 (q, J=6.0 Hz, 2H), 3.18 (d, J=5.1 Hz, 2H), 2.44 (t, J=6.2 Hz, 2H). ¹⁹F NMR (471 MHz, DMSO) δ −119.16 [−121.38]. m/z (ES⁺), [M+H]⁺ 465.7. HPLC (A05) t_{R}=1.85 min.

### Preparation of Compound A-153 (N-[5-(5-fluoro-1H-benzimidazol-2-yl)-1-methyl-pyrazol-3-yl]-6-(4-methoxy-1-piperidyl)pyridine-3-carboxamide)

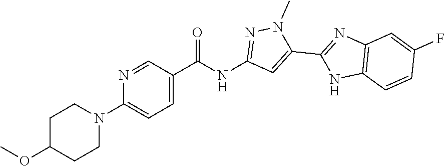 [SMILES: COC1CCN(c2ccc(C(=O)Nc3cc(-c4nc5cc(F)ccc5[nH]4)n(C)n3)cn2)CC1]

6-Chloro-N-[5-(5-fluoro-1H-benzimidazol-2-yl)-1-methyl-pyrazol-3-yl]pyridine-3-carboxamide (L-12) (40 mg, 0.108 mmol) was dissolved in DMSO (1 mL). DIEA (37.6 μL, 0.216 mmol) and 4-methoxypiperidine (20.0 μL, 0.162 mmol) were added. The mixture was heated at 100° C. for 18 h. Water (15 mL) was added, and the mixture was stirred at 23° C. for 5 min. The mixture was filtered, and the solid was purified by silica gel chromatography (12 g cartridge) eluting with DCM and MeOH (0-10%) to provide the title compound as a solid in a 1:1 mixture of benzimidazole tautomers (27.9 mg, 58%). ¹H NMR (400 MHz, DMSO) δ 13.21 (s, 1H), 10.83 (s, 1H), 8.79 (dd, J=2.5, 0.7 Hz, 1H), 8.14 (dd, J=9.1, 2.5 Hz, 1H), 7.79-7.68 [7.58-7.50] (m, 1H), 7.58-7.50 [7.36-7.32] (m, 1H), 7.48 (s, 1H), 7.17-7.06 (m, 1H), 6.91 (d, J=9.1 Hz, 1H), 4.29 (s, 3H), 4.16-3.94 (m, 2H), 3.58-3.41 (m, 1H), 3.36-3.30 (m, 2H), 3.30 (s, 3H), 1.98-1.77 (m, 2H), 1.62-1.30 (m, 2H). ¹⁹F NMR (376 MHz, DMSO) δ −118.81 [−121.04]. m/z (ES⁺), [M+H]⁺ 450.6. HPLC (A05) t_{R}=2.26 min.

### Preparation of Compound A-154 (N-[5-(7-fluoro-1H-benzimidazol-2-A-1-methyl-pyrazol-3-yl]-6-[4-(2-methoxyethyl)piperazin-1-yl]pyridine-3-carboxamide)

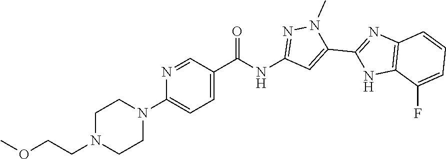 [SMILES: COCCN1CCN(c2ccc(C(=O)Nc3cc(-c4nc5cccc(F)c5[nH]4)n(C)n3)cn2)CC1]

1-(2-Methoxyethyl)piperazine (0.0233 g, 0.162 mmol) was added to a solution of 6-chloro-N-[5-(7-fluoro-1H-benzimidazol-2-yl)-1-methyl-pyrazol-3-yl]pyridine-3-carboxamide (L-13) (50 mg, 0.135 mmol) and DIEA (0.0462 mL, 0.270 mmol) in DMSO (1 mL) The mixture was stirred at 100° C. for 72 h. EtOAc (100 mL) was added, and the organic phase was washed with water (3×70 mL) and brine (50 mL), dried (MgSO₄), filtered, and concentrated. The residue was purified by silica gel chromatography (12 g cartridge) eluting with DCM and MeOH (0-20%) and by silica gel chromatography (4 g cartridge) eluting with DCM and MeOH (0-10%) +1% NEt₃ to provide the title compound as a solid in a 4:1 mixture of tautomers (24.2 mg, 38%). ¹H NMR (400 MHz, DMSO) δ [13.56] 13.37 (s, 1H), 10.86 [10.83] (s, 1H), 8.80 (d, J=2.4 Hz, 1H), 8.15 (dd, J=9.1, 2.4 Hz, 1H), [7.61] 7.50 (s, 1H), [7.57] 7.37 (d, J=8.1 Hz, 1H), 7.28-7.19 (m, 1H), [7.12] 7.05 (dd, J=11.1, 7.9 Hz, 1H), 6.89 (d, J=9.2 Hz, 1H), 4.30 [4.29] (s, 3H), 3.62 (s, 4H), 3.48 (t, J=5.2 Hz, 2H), 3.25 (s, 3H), 2.58-2.51 (m, 4H), [1.23] 1.17 (t, J=7.3 Hz, 2H). ¹⁹F NMR (376 MHz, DMSO) δ −128.69 (s). m/z (ES⁺), [M+H]⁺ 479.7. HPLC (A05) t_{R}=1.92 min.

### Preparation of Compound A-157 (N-[5-(7-fluoro-1H-benzimidazol-2-A-1-methyl-pyrazol-3-yl]-6-(4-methoxy-1-piperidyl)pyridine-3-carboxamide)

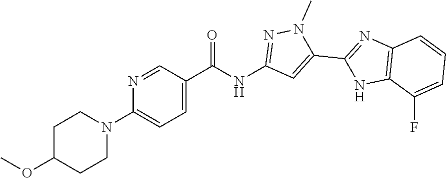 [SMILES: COC1CCN(c2ccc(C(=O)Nc3cc(-c4nc5cccc(F)c5[nH]4)n(C)n3)cn2)CC1]

4-Methoxypiperidine (0.0205 g, 0.178 mmol) was added to a solution of 6-chloro-N-[5-(7-fluoro-1H-benzimidazol-2-yl)-1-methyl-pyrazol-3-yl]pyridine-3-carboxamide (L-13) (60 mg, 0.162 mmol) and DIEA (0.0554 mL, 0.324 mmol) in DMSO (1 mL) The mixture was stirred at 100° C. for 18 h. Water (20 mL) was added, and the mixture was filtered. The solid was purified by silica gel chromatography (12 g cartridge) eluting with DCM and MeOH (0-5%) to provide the title compound as a solid (38.2 mg, 53%). ¹H NMR (500 MHz, DMSO) δ 13.36 (s, 1H), 10.82 (s, 1H), 8.79 (d, J=2.5 Hz, 1H), 8.13 (dd, J=9.1, 2.6 Hz, 1H), 7.49 (s, 1H), 7.37 (d, J=8.1 Hz, 1H), 7.24 (d, J=5.8 Hz, 1H), 7.04 (t, J=9.0 Hz, 1H), 6.90 (d, J=8.9 Hz, 1H), 4.30 (s, 3H), 4.08-4.01 (m, 2H), 3.50-3.42 (m, 1H), 3.33 (dd, J=9.3, 3.5 Hz, 2H), 3.29 (s, 3H), 1.90 (dt, J=12.9, 5.6 Hz, 2H), 1.47-1.38 (m, 2H). ¹⁹F NMR (471 MHz, DMSO) δ −128.65 (s). m/z (ES⁺), [M+H]⁺ 450.2. HPLC (A05) t_{R}=2.27 min.

### Preparation of Compound A-158 (N-[5-(7-fluoro-1H-benzimidazol-2-A-1-methyl-pyrazol-3-yl]-6-[4-(2-hydroxyethyl)piperazin-1-yl]pyridine-3-carboxamide).

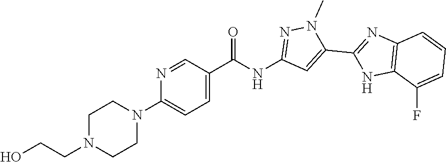 [SMILES: Cn1nc(NC(=O)c2ccc(N3CCN(CCO)CC3)nc2)cc1-c1nc2cccc(F)c2[nH]1]

2-Piperazin-1-ylethanol (0.0210 g, 0.162 mmol) was added to a solution of 6-chloro-N-[5-(7-fluoro-1H-benzimidazol-2-yl)-1-methyl-pyrazol-3-yl]pyridine-3-carboxamide (L-13) (50 mg, 0.135 mmol) and DIEA (0.0462 mL, 0.270 mmol) in DMSO (1 mL) The mixture was stirred at 100° C. for 72 h. EtOAc (100 mL) was added. The organic phase was washed with water (3×70 mL) and brine (50 mL), dried (MgSO₄), filtered, and concentrated. The residue was purified by silica gel chromatography (12 g cartridge) eluting with DCM and MeOH (0-20%) followed by silica gel chromatography (4 g cartridge) eluting with DCM and MeOH (0-10%)+1% NEt₃. The purified residue was dissolved in EtOAc (50 mL), and the organic phase was washed with water (3×20 mL). The organic phase was concentrated to provide the title compound as a solid (12.4 mg, 20%). ¹H NMR (400 MHz, DMSO) δ 13.37 (s, 1H), 10.85 (s, 1H), 8.79 (d, J=2.5 Hz, 1H), 8.14 (dd, J=9.1, 2.5 Hz, 1H), 7.49 (s, 1H), 7.37 (d, J=8.4 Hz, 1H), 7.23 (t, J=11.9 Hz, 1H), 7.12-6.99 (m, 1H), 6.88 (d, J=9.0 Hz, 1H), 4.44 (t, J=5.3 Hz, 1H), 4.30 (s, 3H), 3.66-3.58 (m, 4H), 3.54 (q, J=6.1 Hz, 2H), 2.53-2.51 (m, 4H), 2.43 (t, J=6.2 Hz, 2H). m/z (ES⁺), [M+H]⁺ 465.2. HPLC (A05) t_{R}=1.87 min.

### Preparation of Compound A-160 (N-[5-(5-fluoro-1H-benzimidazol-2-A-1-methyl-pyrazol-3-yl]-6-[4-(oxetan-3-yl)piperazin-1-yl]pyridine-3-carboxamide)

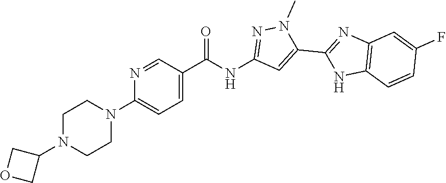 [SMILES: Cn1nc(NC(=O)c2ccc(N3CCN(C4COC4)CC3)nc2)cc1-c1nc2cc(F)ccc2[nH]1]

6-Chloro-N-[5-(5-fluoro-1H-benzimidazol-2-yl)-1-methyl-pyrazol-3-yl]pyridine-3-carboxamide (L-12) (40 mg, 0.108 mmol) was dissolved in DMSO (1 mL). DIEA (37.6 μL, 0.216 mmol) and 4-(4-piperidyl)morpholine (27.6 mg, 0.162 mmol) were added. The mixture was heated at 100° C. for 18 h. Water (15 mL) was added, and the mixture was stirred at 23° C. for 5 min. The mixture was filtered, and the solid was purified by silica gel chromatography (12 g cartridge) eluting with DCM and MeOH (0-10%) to provide the title compound as a solid (23.5 mg, 46%). ¹H NMR (500 MHz, DMSO) δ 13.20 (s, 1H), 10.84 (s, 1H), 8.81 (d, J=2.6 Hz, 1H), 8.16 (dd, J=9.1, 2.6 Hz, 1H), 7.70-7.57 (m, 1H), 7.47 (s, 1H), 7.45-7.36 (m, 1H), 7.14-7.07 (m, 1H), 6.91 (d, J=9.0 Hz, 1H), 4.58 (t, J=6.5 Hz, 2H), 4.49 (t, J=6.0 Hz, 2H), 4.29 (s, 3H), 3.67 (t, J=5.0 Hz, 4H), 3.45 (p, J=6.2 Hz, 1H), 2.36 (t, J=5.0 Hz, 4H). ^{19F} NMR (471 MHz, DMSO) δ −118.89 [−121.14]. m/z (ES⁺), [M+H]⁺ 477.6. HPLC (A05) t_{R}=2.03 min.

### Preparation of Compound A-168 (N-[5-(5-fluoro-1H-benzimidazol-2-yl)-1-methyl-pyrazol-3-yl]-6-(4-morpholino-1-piperidyl)pyridine-3-carboxamide)

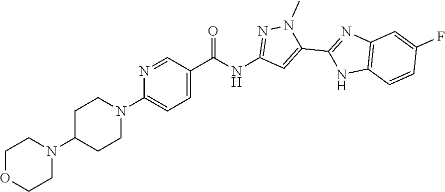 [SMILES: Cn1nc(NC(=O)c2ccc(N3CCC(N4CCOCC4)CC3)nc2)cc1-c1nc2cc(F)ccc2[nH]1]

6-Chloro-N-[5-(5-fluoro-1H-benzimidazol-2-yl)-1-methyl-pyrazol-3-yl]pyridine-3-carboxamide (L-12) (40 mg, 0.108 mmol) was dissolved in DMSO (1 mL). DIEA (37.6 μL, 0.216 mmol) and 4-(4-piperidyl)morpholine (27.6 mg, 0.162 mmol) were added. The mixture was heated at 100° C. for 18 h. Water (15 mL) was added, and the mixture was stirred at 23° C. for 5 min. The mixture was filtered, and the solid was purified by silica gel chromatography (12 g cartridge) eluting with DCM and MeOH (0-10%) to provide the title compound as a solid (36.9 mg, 68%). ¹H NMR (500 MHz, DMSO) δ 13.20 (s, 1H), 10.82 (s, 1H), 8.79 (d, J=2.5 Hz, 1H), 8.13 (dd, J=9.0, 2.5 Hz, 1H), 7.78-7.68 [7.61-7.50] (m, 1H), 7.61-7.50 [7.40-7.27] (m, 1H), 7.48 (s, 1H), 7.23-7.04 (m, 1H), 6.90 (d, J=9.1 Hz, 1H), 4.52-4.38 (m, 2H), 4.29 (s, 3H), 3.57 (t, J=4.6 Hz, 4H), 3.08-2.84 (m, 2H), 2.51-2.45 (m, 4H), 2.47-2.41 (m, 1H), 2.04-1.68 (m, 2H), 1.58-1.28 (m, 2H). ¹⁹F NMR (471 MHz, DMSO) δ −118.75 [−120.99]. m/z (ES⁺), [M+H]⁺ 505.7. HPLC (A05) t_{R}=1.91 min.

### Preparation of Compound A-173 (N-[5-(7-fluoro-1H-benzimidazol-2-A-1-methyl-pyrazol-3-yl]-6-(4-morpholino-1-piperidyl)pyridine-3-carboxamide)

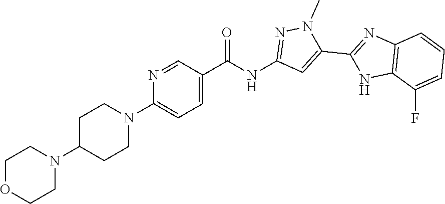 [SMILES: Cn1nc(NC(=O)c2ccc(N3CCC(N4CCOCC4)CC3)nc2)cc1-c1nc2cccc(F)c2[nH]1]

4-(4-Piperidyl)morpholine (0.0207 g, 0.121 mmol) was added to a solution of 6-chloro-N-[5-(7-fluoro-1H-benzimidazol-2-yl)-1-methyl-pyrazol-3-yl]pyridine-3-carboxamide (L-13) (45 mg, 0.121 mmol) and DIEA (0.0416 mL, 0.243 mmol) in DMSO (1 mL). The mixture was stirred at 100° C. for 18 h. Water (20 mL) was added, and the mixture was filtered. The solid was purified by silica gel chromatography (12 g cartridge) eluting with DCM and MeOH (0-10%) to provide the title compound as a solid (34.7 mg, 57%). ¹H NMR (400 MHz, DMSO) δ 13.33 (s, 1H), 10.79 (s, 1H), 8.75 (d, J=2.5 Hz, 1H), 8.09 (dd, J=9.1, 2.5 Hz, 1H), 7.46 (s, 1H), 7.33 (d, J=6.6 Hz, 1H), 7.21 (dd, J=12.4, 4.7 Hz, 1H), 7.04-6.97 (m, 1H), 6.86 (d, J=9.4 Hz, 1H), 4.41 (d, J=13.3 Hz, 2H), 4.26 (s, 3H), 3.56-3.51 (m, 4H), 2.88 (t, J=11.4 Hz, 2H), 2.51 (dd, J=6.6, 4.1 Hz, 1H), 2.43 (dd, J=9.9, 5.5 Hz, 4H), 1.83 (d, J=11.6 Hz, 2H), 1.37-1.26 (m, 2H). m/z (ES⁺), [M+H]⁺ 505.3. HPLC (A05) t_{R}=1.91 min.

### Preparation of Compound A-176 (N-[5-(1H-benzimidazol-2-yl)-1-(2-methoxyethyl)pyrazol-3-yl]-6-(3-methoxyazetidin-1-yl)pyridine-3-carboxamide)

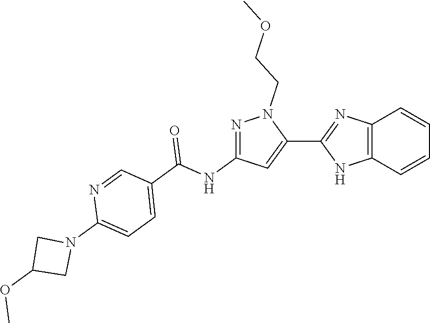 [SMILES: COCCn1nc(NC(=O)c2ccc(N3CC(OC)C3)nc2)cc1-c1nc2ccccc2[nH]1]

DIEA (0.0808 mL, 0.464 mmol) was added to a solution of 3-methoxy azetidine hydrochloride (42.4 mg, 0.348 mmol) and N-[5-(1H-benzimidazol-2-yl)-1-(2-methoxyethyl)pyrazol-3-yl]-6-chloro-pyridine-3-carboxamide (L-20) (92 mg, 0.232 mmol) in DMF (2 mL). The mixture was heated at 100° C. for 20 h. The mixture was cooled to 23° C., and water (20 mL) was added. The aq. phase was extracted with EtOAc (3×10 mL). The combined organic phases were washed with sat. NaHCO₃ (10 mL) and brine (10 mL), dried (MgSO₄), filtered, and concentrated under reduced pressure. The residue was purified by preparative HPLC (BEH C18 30×150) eluting with water (10 mM (NH₄)(HCO₃)) and MeCN (31-41%) to provide the title compound as a solid (31 mg, 30%). ¹H NMR (400 MHz, DMSO) δ 13.05 (s, 1H), 10.88 (s, 1H), 8.78 (d, J=2.2 Hz, 1H), 8.15 (dd, J=8.8, 2.3 Hz, 1H), 7.69 (s, 1H), 7.54 (s, 1H), 7.48 (s, 1H), 7.25 (s, 2H), 6.43 (d, J=8.8 Hz, 1H), 5.00 (t, J=5.7 Hz, 2H), 4.35 (d, J=6.3 Hz, 1H), 4.27-4.20 (m, 2H), 3.85 (dd, J=9.5, 3.5 Hz, 2H), 3.80 (t, J=5.7 Hz, 2H), 3.27 (s, 3H), 3.21 (s, 3H). m/z (ES⁺), [M−H]⁻ 446.6. HPLC (A05) t_{R}=2.12 min).

### Preparation of Compound A-178 (N-[5-(1H-benzimidazol-2-yl)-1-(2-methoxyethyl)pyrazol-3-yl]-6-[4-(2-methoxyethyl)piperazin-1-yl]pyridine-3-carboxamide)

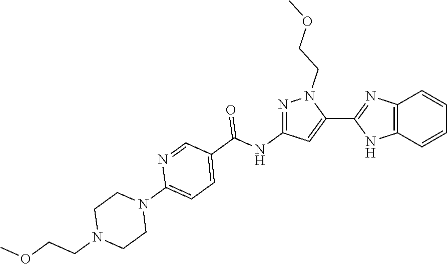 [SMILES: COCCN1CCN(c2ccc(C(=O)Nc3cc(-c4nc5ccccc5[nH]4)n(CCOC)n3)cn2)CC1]

DIEA (0.0808 mL, 0.464 mmol) was added to a solution of 1-(2-methoxyethyl)-piperazine (50.2 mg, 0.348 mmol) and N-[5-(1H-benzimidazol-2-yl)-1-(2-methoxyethyl)pyrazol-3-yl]-6-chloro-pyridine-3-carboxamide (L-20) (92 mg, 0.232 mmol) in DMF (2 mL). The mixture was heated at 100° C. for 20 h. The mixture was cooled to 23° C. and water (20 mL) was added. The aq. phase was extracted with EtOAc (3×10 mL). The combined organic phases were washed with sat. NaHCO₃ (10 mL) and brine (10 mL), dried (MgSO₄), filtered, and concentrated. The residue was purified by silica gel chromatography (40 g cartridge) eluting with DCM and MeOH (0-10%) to provide the title compound as a solid (77 mg, 66%). ¹H NMR (400 MHz, CD3OD) δ 8.75 (d, J=2.0 Hz, 1H), 8.10 (dd, J=9.1, 2.6 Hz, 1H), 7.65 (s, 2H), 7.29 (dd, J=6.2, 3.1 Hz, 2H), 7.27 (s, 1H), 6.87 (d, J=8.6 Hz, 1H), 4.89 (t, J=5.5 Hz, 2H), 3.83 (t, J=5.5 Hz, 2H), 3.74-3.70 (m, 4H), 3.59 (t, J=5.5 Hz, 2H), 3.36 (s, 3H), 3.24 (s, 3H), 2.64 (dd, J=7.3, 3.7 Hz, 6H). m/z (ES⁺), [M−H]⁻ 503.8. HPLC (A05) t_{R}=1.93 min).

### Preparation of Compound A-182 (N-[5-(1H-benzimidazol-2-yl)-1-(2-methoxyethyl)pyrazol-3-yl]-6-(4-methyl-piperazin-1-yl)pyridine-3-carboxamide)

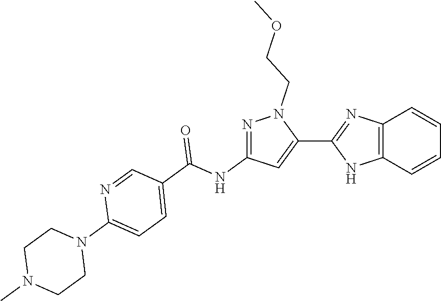 [SMILES: COCCn1nc(NC(=O)c2ccc(N3CCN(C)CC3)nc2)cc1-c1nc2ccccc2[nH]1]

DIEA (0.0878 mL, 0.504 mmol) was added to a solution of 1-methylpiperazine (37.9 mg, 0.378 mmol) and N-[5-(1H-benzimidazol-2-yl)-1-(2-methoxyethyl)pyrazol-3-yl]-6-chloro-pyridine-3-carboxamide (L-20) (100 mg, 0.252 mmol) in DMF (2 mL). The mixture was heated at 100° C. for 20 h. The mixture was cooled to 23° C., and water (20 mL) was added. The aq. phase was extracted with EtOAc (3×10 mL). The combined organic phases were washed with aq. NaHCO₃ (10 mL) and brine (10 mL), dried (MgSO₄), filtered, and concentrated. The residue was purified by preparative HPLC (BEH C18 30×150) eluting with water (10 mM (NH₄)(HCO₃)) and MeCN (36-53%) to provide the title compound as a solid (26 mg, 22%). ¹H NMR (500 MHz, DMSO) δ 13.05 (s, 1H), 10.88 (s, 1H), 8.80 (d, J=2.3 Hz, 1H), 8.16 (dd, J=9.0, 2.5 Hz, 1H), 7.70 (s, 1H), 7.52 (s, 1H), 7.49 (s, 1H), 7.24 (s, 2H), 6.89 (d, J=9.0 Hz, 1H), 5.00 (t, J=5.7 Hz, 2H), 3.80 (t, J=5.7 Hz, 2H), 3.65-3.61 (m, 4H), 3.21 (s, 3H), 2.42-2.37 (m, 4H), 2.22 (s, 3H). m/z (ES⁺), [M−H]⁻ 459.6. HPLC (A05) t_{R}=1.89 min.

### Preparation of Compound A187 (N-[5-(7-fluoro-5-methoxy-1H-benzimidazol-2-yl)-1-methyl-pyrazol-3-yl]-6-(3-methoxyazetidin-1-yl)pyridine-3-carboxamide)

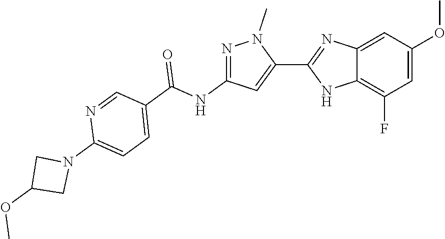 [SMILES: COc1cc(F)c2[nH]c(-c3cc(NC(=O)c4ccc(N5CC(OC)C5)nc4)nn3C)nc2c1]

DIEA (0.0217 mL, 0.125 mmol) was added to a solution of 3-methoxyazetidine hydrochloride (11.5 mg, 0.0936 mmol) and 6-chloro-N-[5-(7-fluoro-5-methoxy-1H-benzimidazol-2-yl)-1-methyl-pyrazol-3-yl]pyridine-3-carboxamide (L-14) (25 mg, 0.0624 mmol) in DMF (1 mL). The mixture was heated at 100° C. for 20 h. The mixture was cooled to 23° C., and water (10 mL) was added. The aq. phase was extracted with EtOAc (3×10 mL). The combined organic phases were washed with sat. NaHCO₃ (10 mL) and brine (10 mL), dried (MgSO₄), filtered, and concentrated. The residue was purified by silica gel chromatography (7 g cartridge) eluting with DCM and MeOH (0-10%). The product was purified by preparative HPLC (BEH C18 30×150) eluting with water (10 mM (NH₄)(HCO₃)) and MeCN (37-47%) to provide the title compound as a solid (16 mg, 57%). ¹H NMR (400 MHz, DMSO) δ 10.84-10.79 (m, 1H), 8.77 (d, J=2.4 Hz, 1H), 8.13 (dd, J=8.8, 2.4 Hz, 1H), 7.43 (s, 1H), 6.86 (d, J=2.0 Hz, 1H), 6.72 (dd, J=12.4, 2.1 Hz, 1H), 6.43 (d, J=8.9 Hz, 1H), 4.35 (d, J=6.3 Hz, 1H), 4.27 (s, 3H), 4.23 (dd, J=8.8, 6.7 Hz, 2H), 3.85 (dd, J=10.4, 3.9 Hz, 2H), 3.82 (s, 3H), 3.27 (s, 3H). m/z (ES), [M−H]⁻ 450.8. HPLC (A05) t_{R}=2.15 min.

### Preparation of Compound A-189 (N-[5-(7-fluoro-5-methoxy-1H-benzimidazol-2-yl)-1-methyl-pyrazol-3-yl]-6-(4-methoxy-1-piperidyl)pyridine-3-carboxamide)

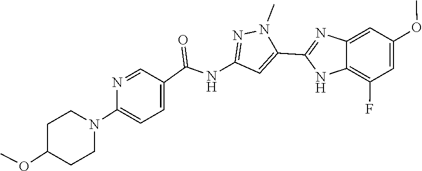 [SMILES: COc1cc(F)c2[nH]c(-c3cc(NC(=O)c4ccc(N5CCC(OC)CC5)nc4)nn3C)nc2c1]

DIEA (0.0217 mL, 0.125 mmol) was added to a solution of 4-methoxypiperidine (10.8 mg, 0.0936 mmol) and 6-chloro-N-[5-(7-fluoro-5-methoxy-1H-benzimidazol-2-yl)-1-methyl-pyrazol-3-yl]-pyridine-3-carboxamide (L-14) (25 mg, 0.0624 mmol) in DMF (1 mL). The mixture was heated at 100° C. for 20 h. The mixture was cooled to 23° C., and water (10 mL) was added. The aq. phase was extracted with EtOAc (3×10 mL). The combined organic phases were washed with sat NaHCO₃ (10 mL) and brine (10 mL), dried (MgSO₄), filtered, and concentrated. The residue was purified by preparative HPLC (BEH C18 30 ×150) eluting with water (10 mM (NH₄)(HCO₂)) and MeCN (42-52%) to provide the title compound as a solid (12 mg, 37%). ¹H NMR (400 MHz, DMSO) δ 10.81 (s, 1H), 8.78 (d, J=2.5 Hz, 1H), 8.41 (s, 1H), 8.13 (dd, J=9.1, 2.5 Hz, 1H), 7.46 (s, 1H), 6.93-6.85 (m, 2H), 6.75 (dd, J=12.4, 2.1 Hz, 1H), 4.27 (s, 3H), 4.04 (d, J=14.3 Hz, 2H), 3.83 (s, 3H), 3.49-3.43 (m, 1H), 3.35-3.30 (m, 2H), 3.29 (s, 3H), 1.89 (s, 2H), 1.43 (dd, J=11.3, 6.6 Hz, 2H). m/z (ES⁺), [M−H]⁻ 478.7. HPLC (A05) t_{R}=2.20 min.

### Preparation of Compound A-190 (N-[5-(7-fluoro-5-methoxy-1H-benzimidazol-2-yl)-1-methyl-pyrazol-3-yl]-6-[4-(2-methoxyethyl)piperazin-1-yl]pyridine-3-carboxamide)

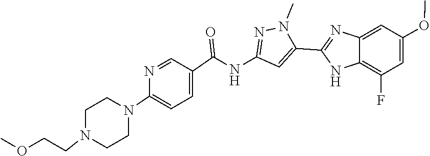

DIEA (0.0217 mL, 0.125 mmol) was added to a solution of 1-(2-methoxyethyl)piperazine (13.5 mg, 0.0936 mmol) and N-[5-(1H-benzimidazol-2-yl)-1-methyl-pyrazol-3-yl]-6-chloro-pyridine-3-carboxamide (L-14) (25 mg, 0.0624 mmol) in DMF (1 mL). The mixture was heated at 100° C. for 20 h. The mixture was cooled to 23° C., and water (10 mL) was added. The aq. phase was extracted with EtOAc (3×10 mL). The combined organic phases were washed with sat. NaHCO₃ (10 mL) and brine (10 mL), dried (MgSO₄), filtered, and concentrated. The residue was purified by preparative HPLC (BEH C18 30 ×150) eluting with water (10 mM (NH₄)(HCO₃)) and MeCN (35-45%) to provide the title compound as a solid (17 mg, 54%). ¹H NMR (400 MHz, CD3OD) δ 8.74 (d, J=2.5 Hz, 1H), 8.09 (dd, J=9.1, 2.6 Hz, 1H), 7.24 (s, 1H), 6.87 (d, J=8.6 Hz, 2H), 6.68 (d, J=11.9 Hz, 1H), 4.24 (s, 3H), 3.87 (s, 3H), 3.74-3.69 (m, 4H), 3.59 (t, J=5.5 Hz, 2H), 3.37 (s, 3H), 2.65 (dd, J=7.5, 3.5 Hz, 6H). m/z (ES), [M−H]⁻ 507.6. HPLC (A05) t_{R}=1.83 min.

### Preparation of Compound A-191 (N-[5-(7-fluoro-5-methoxy-1H-benzimidazol-2-yl)-1-methyl-pyrazol-3-yl]-6-(4-methylpiperazin-1-yl)pyridine-3-carboxamide)

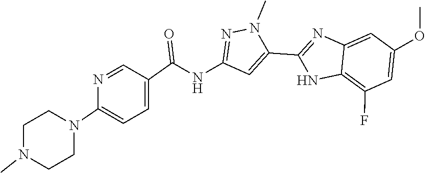

DIEA (0.0217 mL, 0.125 mmol) was added to a solution of 1-methylpiperazine (9.37 mg, 0.0936 mmol) and 6-chloro-N-[5-(7-fluoro-5-methoxy-1H-benzimidazol-2-yl)-1-methyl-pyrazol-3-yl]pyridine-3-carboxamide (L-14) (25 mg, 0.0624 mmol) in DMF (1 mL). The mixture was heated at 100° C. for 20 h. The mixture was cooled to 23° C., and water (10 mL) was added. The aq. phase was extracted with EtOAc (3×10 mL). The combined organic phases were washed with sat. NaHCO₃ (10 mL) and brine (10 mL), dried (MgSO₄), filtered, and concentrated. The residue was purified by silica gel chromatography (7 g cartridge) eluting with DCM and MeOH (0-10%). The product was dissolved in DMSO (0.500 mL) and NEt₃ (0.500 mL). Water (10 mL) was added. The aqueous phase was extracted with EtOAc (3×10 mL). The combined organic phases were washed with NaHCO₃ (3×10 mL) and brine (10 mL), dried (MgSO₄), filtered, and concentrated to provide the title compound as a solid (20 mg, 69%). ¹H NMR (400 MHz, DMSO) δ 13.46-13.36 (m, 1H), 10.79 (s, 1H), 8.79 (d, J=2.8 Hz, 1H), 8.14 (dd, J=9.1, 2.5 Hz, 1H), 7.43 (s, 1H), 6.89 (d, J=9.3 Hz, 1H), 6.86-6.63 (m, 2H), 4.26 (s, 3H), 3.82 (s, 3H), 3.67-3.56 (m, 4H), 2.41-2.35 (m, 4H), 2.22 (s, 3H). m/z (ES), [M−H]⁻ 463.6. HPLC (A05) t_{R}=1.85 min

### Preparation of Compound A-203 (N-[5-(1H-benzimidazol-2-yl)-1-methyl-pyrazol-3-yl]-6-[4-(oxetan-3-yl)piperazin-1-yl]pyridine-3-carboxamide)

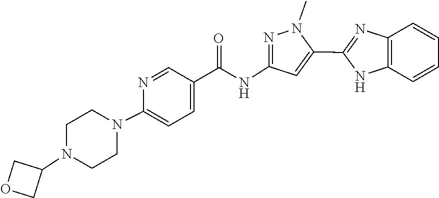 [SMILES: Cn1nc(NC(=O)c2ccc(N3CCN(C4COC4)CC3)nc2)cc1-c1nc2ccccc2[nH]1]

1-(Oxetan-3-yl)piperazine (0.0161 g, 0.113 mmol) was added to a solution of N-[5-(1H-benzimidazol-2-yl)-1-methyl-pyrazol-3-yl]-6-chloro-pyridine-3-carboxamide (L-11) (40 mg, 0.113 mmol) and DIEA (0.0388 mL, 0.227 mmol) in DMSO (1 mL). The mixture was stirred at 100° C. for 18 h. Water (20 mL) was added, and the mixture was filtered. The solid was purified by preparative HPLC (BEH C18 30×100) eluting with water (10 mM (NH₄)(HCO₂)) and MeCN (21-37%) to provide the title compound as a solid (24.7 mg, 48%). ¹H NMR (400 MHz, DMSO) δ 13.05-11.89 (m, 1H), 10.84 (s, 1H), 8.80 (d, J=2.2 Hz, 1H), 8.15 (dd, J=9.1, 2.5 Hz, 1H), 7.77-7.52 (m, 2H), 7.47 (s, 1H), 7.25 (dd, J=6.0, 3.0 Hz, 2H), 6.90 (d, J=9.1 Hz, 1H), 4.57 (t, J=6.5 Hz, 2H), 4.48 (t, J=6.1 Hz, 2H), 4.30 (s, 3H), 3.70-3.62 (m, 4H), 3.47-3.40 (m, 1H), 2.38-2.30 (m, 4H). m/z (ES⁺), [M+H]⁺ 459.4. HPLC (A05) t_{R}=1.97 min.

### Preparation of Compound A-204 (N-[5-(1H-benzimidazol-2-yl)-1-methyl-pyrazol-3-yl]-6-[4-(2-hydroxy-ethyl)piperazin-1-yl]pyridine-3-carboxamide)

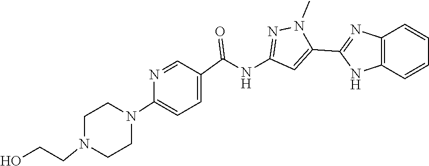 [SMILES: Cn1nc(NC(=O)c2ccc(N3CCN(CCO)CC3)nc2)cc1-c1nc2ccccc2[nH]1]

2-Piperazin-1-ylethanol (0.0148 g, 0.113 mmol) was added to a solution of N-[5-(1H-benzimidazol-2-yl)-1-methyl-pyrazol-3-yl]-6-chloro-pyridine-3-carboxamide (L-11) (40 mg, 0.113 mmol) and DIEA (0.0388 mL, 0.227 mmol) in DMSO (1 mL). The mixture was heated at 100° C. for 18 h. Water (20 mL) was added, and the mixture was filtered. The solid was purified by preparative HPLC (BEH C18 30×100) eluting with water (10 mM (NH₄)(HCO₂)) and MeCN (5-65%). The purified residue was dissolved in EtOAc (30 mL). The organic phase was washed with water (3×15 mL) and concentrated to provide the title compound as a solid (6.6 mg, 13%).

¹H NMR (400 MHz, DMSO) δ 13.05 (s, 1H), 10.83 (s, 1H), 8.79 (d, J=2.6 Hz, 1H), 8.14 (dd, J=9.1, 2.5 Hz, 1H), 7.71 (d, J=7.4 Hz, 1H), 7.53 (d, J=7.8 Hz, 1H), 7.47 (s, 1H), 7.30-7.20 (m, 2H), 6.88 (d, J=9.1 Hz, 1H), 4.44 (t, J=5.4 Hz, 1H), 4.30 (s, 3H), 3.65-3.58 (m, 4H), 3.54 (q, J=6.1 Hz, 2H), 2.52 (d, J=6.8 Hz, 4H), 2.44 (t, J=6.2 Hz, 2H). m/z (ES⁺), [M+H]⁺ 447.4. HPLC (A05) t_{R}=1.81 min.

### Preparation of Compound A-205 (N-[5-(1H-benzimidazol-2-yl)-1-methyl-pyrazol-3-yl]-5-(4-morpholino-1-piperidyl)pyrazine-2-carboxamide)

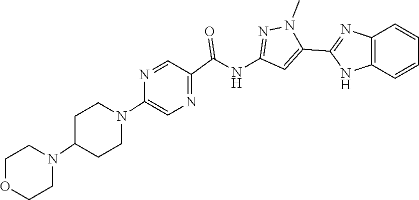 [SMILES: Cn1nc(NC(=O)c2cnc(N3CCC(N4CCOCC4)CC3)cn2)cc1-c1nc2ccccc2[nH]1]

4-(4-Piperidyl)morpholine (40.5 mg, 0.238 mmol) was added to a solution of N-[5-(1H-benzimidazol-2-yl)-1-methyl-pyrazol-3-yl]-5-(triazolo[4,5-b]pyridin-3-yloxy)pyrazine-2-carboxamide (L-28) (77 mg, 0.170 mmol) in DMSO (2 mL). The mixture was heated at 110° C. for 18 h. Water (20 mL) was added, and the mixture was filtered. The solid was purified by silica gel chromatography (12 g cartridge) eluting with DCM and MeOH (0-10%) to provide the title compound as a solid (38.9 mg, 47%). ¹H NMR (400 MHz, DMSO) δ 13.09 (s, 1H), 10.12 (s, 1H), 8.73 (d, J=1.3 Hz, 1H), 8.38 (d, J=1.1 Hz, 1H), 7.75-7.69 (m, 1H), 7.58-7.50 (m, 1H), 7.45 (s, 1H), 7.32-7.20 (m, 2H), 4.52 (d, J=13.4 Hz, 2H), 4.29 (s, 3H), 3.60-3.54 (m, 4H), 3.05 (t, J=13.7 Hz, 2H), 2.49-2.45 (m, 4H), 1.90 (d, J=10.5 Hz, 2H), 1.42 (dd, J=20.8, 11.3 Hz, 2H), 1.28-1.21 (m, 1H). m/z (ES⁺), [M+H]⁺ 488.4. HPLC (A05) t_{R}=1.91 min.

### Preparation of Compound A-206 (N-[5-(1H-benzimidazol-2-yl)-1-methyl-pyrazol-3-yl]-2-(4-morpholino-1-piperidyl)pyrimidine-5-carboxamide)

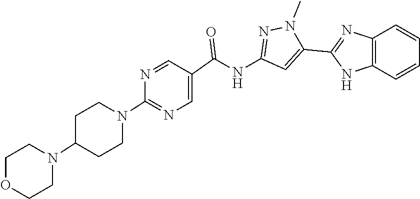 [SMILES: Cn1nc(NC(=O)c2cnc(N3CCC(N4CCOCC4)CC3)nc2)cc1-c1nc2ccccc2[nH]1]

4-(4-Piperidyl)morpholine (36.8 mg, 0.216 mmol) was added to a solution of N-[5-(1H-benzimidazol-2-yl)-1-methyl-pyrazol-3-yl]-2-(triazolo[4,5-b]pyridin-3-yloxy)pyrimidine-5-carboxamide (70 mg, 0.154 mmol) (L-28) (70 mg, 0.154 mmol) in DMSO (2 mL). The mixture was heated at 110° C. for 18 h. Water (20 mL) was added, and the mixture was filtered. The solid was purified by silica gel chromatography (12 g cartridge) eluting with DCM and MeOH (0-10%) to provide the title compound as a solid (37.1 mg, 49%). ¹H NMR (400 MHz, DMSO) δ 13.03 (s, 1H), 10.92 (s, 1H), 8.90 (s, 2H), 7.68 (d, J=6.0 Hz, 1H), 7.49 (d, J=8.5 Hz, 1H), 7.43 (s, 1H), 7.20 (dd, J=17.4, 7.4 Hz, 2H), 4.71 (d, J=13.0 Hz, 2H), 4.27 (s, 3H), 3.55-3.51 (m, 4H), 2.98 (t, J=12.0 Hz, 2H), 2.44 (dd, J=3.4, 2.7 Hz, 4H), 1.84 (d, J=14.8 Hz, 2H), 1.31 (q, J=10.2 Hz, 2H), 1.22-1.14 (m, 1H). m/z (ES⁺), [M+H]⁺ 488.4. HPLC (A05) t_{R}=1.86 min.

### Preparation of Compound A-207 (N-[5-(1H-benzimidazol-2-yl)-1-methyl-pyrazol-3-yl]-6-(4-morpholino-1-piperidyl)pyridazine-3-carboxamide)

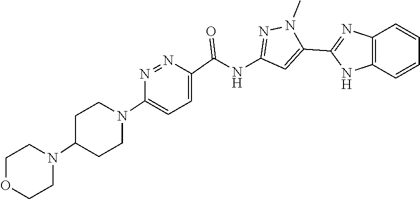 [SMILES: Cn1nc(NC(=O)c2ccc(N3CCC(N4CCOCC4)CC3)nn2)cc1-c1nc2ccccc2[nH]1]

4-(4-Piperidyl)morpholine (0.0404 g, 0.237 mmol) was added to a solution of N-[5-(1H-benzimidazol-2-yl)-1-methyl-pyrazol-3-yl]-6-chloro-pyridazine-3-carboxamide (L-29) (60 mg, 0.170 mmol) in DMSO (2 mL). The mixture was heated at 110° C. for 18 h. Water (20 mL) was added, and the mixture was filtered. The solid was purified by silica gel chromatography (12 g cartridge) eluting with DCM and MeOH (0-15%) to provide the title compound as a solid (50.3 mg, 61%). ¹H NMR (400 MHz, DMSO) δ 13.09 (s, 1H), 10.56 (s, 1H), 7.94 (d, J=9.6 Hz, 1H), 7.73 (d, J=6.3 Hz, 1H), 7.54 (d, J=6.5 Hz, 1H), 7.46 (s, 1H), 7.42 (d, J=9.7 Hz, 1H), 7.25 (t, J=9.7 Hz, 2H), 4.53 (d, J=13.1 Hz, 2H), 4.30 (s, 3H), 3.59-3.54 (m, 4H), 3.07 (t, J=11.5 Hz, 2H), 2.53 (dd, J=6.4, 2.5 Hz, 1H), 2.47 (s, 4H), 1.91 (d, J=10.1 Hz, 2H), 1.43 (qd, J=12.8, 4.5 Hz, 2H). m/z (ES⁺), [M+H]⁺ 488.5. HPLC (A05) t_{R}=1.90 min.

### Preparation of Compound A-208 (N-[5-(1H-benzimidazol-2-yl)-1-methyl-pyrazol-3-yl]-6-(4-morpholino-1-piperidyl)pyridine-3-carboxamide)

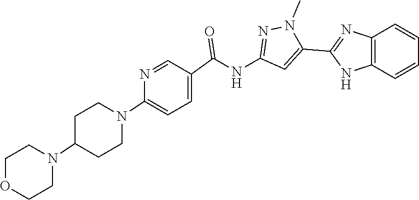 [SMILES: Cn1nc(NC(=O)c2ccc(N3CCC(N4CCOCC4)CC3)nc2)cc1-c1nc2ccccc2[nH]1]

4-(4-Piperidyl)morpholine (0.0241 g, 0.142 mmol) was added to a solution of N-[5-(1H-benzimidazol-2-yl)-1-methyl-pyrazol-3-yl]-6-chloro-pyridine-3-carboxamide (L-11) (50 mg, 0.142 mmol) and DIEA (0.0485 mL, 0.283 mmol) in DMSO (1 mL). The mixture was heated at 100° C. for 72 h. Water (20 mL) was added, and the mixture was filtered. The solid was purified by silica gel chromatography (12 g cartridge) eluting with DCM and MeOH (0-15%) to provide the title compound as a solid (60 mg, 87%). ¹H NMR (400 MHz, DMSO) δ 13.08 (s, 1H), 10.80 (s, 1H), 8.78 (d, J=2.4 Hz, 1H), 8.13 (dd, J=9.1, 2.6 Hz, 1H), 7.71 (d, J=8.1 Hz, 1H), 7.53 (d, J=7.8 Hz, 1H), 7.47 (s, 1H), 7.31-7.19 (m, 2H), 6.89 (d, J=9.0 Hz, 1H), 4.44 (d, J=13.1 Hz, 2H), 4.30 (s, 3H), 3.59-3.53 (m, 4H), 2.92 (t, J=11.6 Hz, 2H), 2.53-2.51 (m, 1H), 2.48-2.45 (m, 4H), 1.86 (d, J=10.5 Hz, 2H), 1.35 (ddd, J=24.0, 11.7, 4.0 Hz, 2H). m/z (ES⁺), [M+H]⁺ 487.3. HPLC (A05) t_{R}=1.84 min.

### Preparation of Compound A-209 (N-[5-(1H-benzimidazol-2-yl)-1-methyl-pyrazol-3-yl]-6-(4-methyl-piperazin-1-yl)pyridine-3-carboxamide)

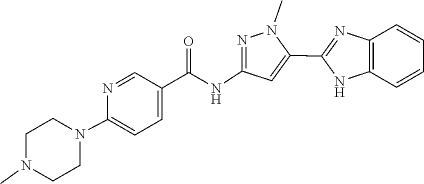 [SMILES: CN1CCN(c2ccc(C(=O)Nc3cc(-c4nc5ccccc5[nH]4)n(C)n3)cn2)CC1]

1-Methylpiperazine (0.0142 g, 0.142 mmol) was added to a solution of N-[5-(1H-benzimidazol-2-yl)-1-methyl-pyrazol-3-yl]-6-chloro-pyridine-3-carboxamide (L-11) (50 mg, 0.142 mmol) and DIEA (0.0485 mL, 0.283 mmol) in DMSO (1 mL). The mixture was heated at 100° C. for 18 h. Water (20 mL) was added, and the mixture was filtered. The solid was purified by silica gel chromatography (12 g cartridge) eluting with DCM and MeOH (0-15%) to provide the title compound as a solid (50.2 mg, 85%). ¹H NMR (400 MHz, DMSO) δ 13.02 (s, 1H), 10.80 (s, 1H), 8.76 (d, J=2.1 Hz, 1H), 8.11 (dd, J=9.1, 2.5 Hz, 1H), 7.71-7.65 (m, 1H), 7.52-7.48 (m, 1H), 7.44 (s, 1H), 7.27-7.16 (m, J=16.4, 7.2, 1.3 Hz, 2H), 6.86 (d, J=9.2 Hz, 1H), 4.26 (s, 3H), 3.65-3.53 (m, 4H), 2.42-2.31 (m, J=8.8 Hz, 4H), 2.20 (s, 3H). m/z (ES⁺), [M+H]⁺ 417.2. HPLC (A05) t_{R}=1.81 min.

### Preparation of Compound A-210 (N-[5-(1H-benzimidazol-2-yl)-1-methyl-pyrazol-3-yl]-6-morpholino-pyridine-3-carboxamide)

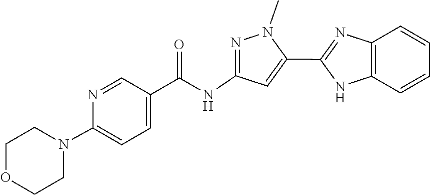 [SMILES: Cn1nc(NC(=O)c2ccc(N3CCOCC3)nc2)cc1-c1nc2ccccc2[nH]1]

Morpholine (0.0247 g, 0.283 mmol) was added to a solution of N-[5-(1H-benzimidazol-2-yl)-1-methyl-pyrazol-3-yl]-6-chloro-pyridine-3-carboxamide (L-11) (50 mg, 0.142 mmol) and DIEA (0.0728 mL, 0.425 mmol) in DMSO (1 mL). The mixture was heated at 100° C. for 18 h. Water (20 mL) was added, and the mixture was filtered. The solid was purified by silica gel chromatography (12 g cartridge) eluting with DCM and MeOH (0-15%) to provide the title compound as a solid (42.7 mg, 75%). ¹H NMR (400 MHz, DMSO) δ 13.03 (s, 1H), 10.86 (s, 1H), 8.82 (d, J=2.0 Hz, 1H), 8.18 (dd, J=9.0, 2.5 Hz, 1H), 7.72 (d, J=7.7 Hz, 1H), 7.53 (d, J=8.1 Hz, 1H), 7.47 (s, 1H), 7.30-7.19 (m, 2H), 6.90 (d, J=9.0 Hz, 1H), 4.30 (s, 3H), 3.73-3.68 (m, 4H), 3.63-3.57 (m, 4H). m/z (ES⁺), [M+H]⁺ 404.4. HPLC (A05) t_{R}=2.13 min.

### Preparation of Compound A-211 (N-[5-(7-fluoro-5-methoxy-1H-benzimidazol-2-yl)-1-methyl-pyrazol-3-yl]-6-[4-(2-hydroxyethyl)piperazin-1-yl]pyridine-3-carboxamide)

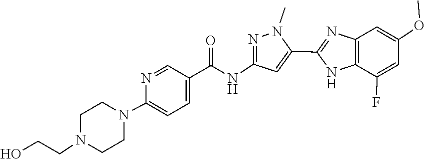 [SMILES: COc1cc(F)c2[nH]c(-c3cc(NC(=O)c4ccc(N5CCN(CCO)CC5)nc4)nn3C)nc2c1]

DIEA (0.0226 mL, 0.130 mmol) was added to a solution of 2-piperazin-1-ylethanol (0.0119 mL, 0.0973 mmol) and 6-chloro-N-[5-(7-fluoro-5-methoxy-1H-benzimidazol-2-yl)-1-methyl-pyrazol-3-yl]pyridine-3-carboxamide (L-14) (26 mg, 0.0649 mmol) in DMF (1 mL). The mixture was heated at 80° C. for 18 h. The mixture was cooled to 23° C., and water (10 mL) was added. The aq. phase was extracted with EtOAc (3×10 mL). The combined organic phases were washed with brine (10 mL), dried (MgSO₄), filtered, and concentrated. The residue was purified by silica gel chromatography (7 g cartridge) eluting with DCM and MeOH (0-10%) to provide the title compound as a solid (13 mg, 41%). ¹H NMR (500 MHz, CD3OD) δ 8.74 (d, J=2.4 Hz, 1H), 8.09 (dd, J=9.1, 2.6 Hz, 1H), 7.24 (s, 1H), 6.87 (d, J=9.0 Hz, 2H), 6.68 (d, J=12.5 Hz, 1H), 4.24 (s, 3H), 3.87 (s, 3H), 3.73 (dd, J=11.1, 5.3 Hz, 6H), 2.68-2.62 (m, 4H), 2.60 (t, J=5.9 Hz, 2H). ¹⁹F NMR (471 MHz, CD3OD) δ −129.30 [−129.52] (s). m/z (ES⁺), [M+H]⁺ 495.5. HPLC (A05) t_{R}=1.90 min.

### Preparation of Compound A-212 (N-[5-(1H-benzimidazol-2-yl)-1-methyl-pyrazol-3-yl]-4-(4-methyl-piperazin-1-yl)benzamide)

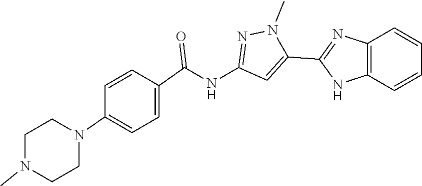 [SMILES: CN1CCN(c2ccc(C(=O)Nc3cc(-c4nc5ccccc5[nH]4)n(C)n3)cc2)CC1]

1-Methylpiperazine (0.400 mL, 3.59 mmol) was added to a solution of N-[5-(1H-benzimidazol-2-yl)-1-methyl-pyrazol-3-yl]-4-fluoro-benzamide (L-16) (50 mg, 0.149 mmol) in DMSO (0.500 mL). The mixture was heated at 110° C. for 72 h. The mixture was cooled to 23° C., and water (20 mL) was added. The mixture was filtered, and the solid was purified by silica gel chromatography (12 g cartridge) eluting with DCM and MeOH (0-20%) to provide the title compound as a solid (42.4 mg, 68%). ¹H NMR (400 MHz, DMSO) δ 13.01 (s, 1H), 10.67 (s, 1H), 7.91 (d, J=9.0 Hz, 2H), 7.67 (d, J=7.4 Hz, 1H), 7.51-7.46 (m, 1H), 7.44 (s, 1H), 7.25-7.16 (m, 2H), 6.95 (d, J=9.1 Hz, 2H), 4.26 (s, 3H), 3.27-3.23 (m, 4H), 2.43-2.38 (m, 4H), 2.19 (s, 3H). m/z (ES⁺), [M+H]⁺ 416.4. HPLC (A05) t_{R}=1.87 min.

### Preparation of Compound A-213 (N-[5-(1H-benzimidazol-2-yl)-1-methyl-pyrazol-3-yl]-4-[4-(2-hydroxy-ethyl)piperazin-1-yl]benzamide)

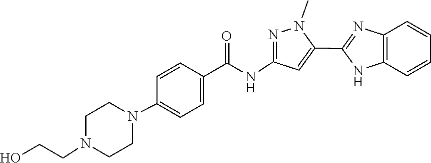 [SMILES: Cn1nc(NC(=O)c2ccc(N3CCN(CCO)CC3)cc2)cc1-c1nc2ccccc2[nH]1]

2-Piperazin-1-ylethanol (0.400 mL, 3.26 mmol) was added to a solution of N-[5-(1H-benzimidazol-2-yl)-1-methyl-pyrazol-3-yl]-4-fluoro-benzamide (L-16) (50 mg, 0.149 mmol) in DMSO (0.500 mL). The mixture was heated to 110° C. and stirred for 72 h. The mixture was cooled to 23° C. and concentrated. The residue was purified by silica gel chromatography (12 g cartridge) eluting with DCM and MeOH (0-20%) and by preparative HPLC (BEH C18 30×100) eluting with water (10 mM (NH₄)(HCO₃)) and MeCN (23-33%) to provide the title compound as a solid (22.4 mg, 34%). ¹H NMR (500 MHz, DMSO) δ 13.05 (s, 1H), 10.71 (s, 1H), 7.95 (d, J=9.0 Hz, 2H), 7.71 (d, J=7.8 Hz, 1H), 7.53 (d, J=7.5 Hz, 1H), 7.48 (s, 1H), 7.25 (dtd, J=16.3, 7.3, 1.2 Hz, 2H), 6.98 (d, J=9.1 Hz, 2H), 4.44 (t, J=5.4 Hz, 1H), 4.30 (s, 3H), 3.54 (q, J=6.1 Hz, 2H), 3.30-3.26 (m, 4H), 2.58-2.54 (m, 4H), 2.44 (t, J=6.2 Hz, 2H). m/z (ES⁺), [M+H]⁺ 446.5. HPLC (A05) t_{R}=1.89 min.

### Preparation of Compound A-214 (N-[5-(7-fluoro-5-methoxy-1H-benzimidazol-2-yl)-1-methyl-pyrazol-3-yl]-6-[4-(oxetan-3-yl)piperazin-1-yl]pyridine-3-carboxamide)

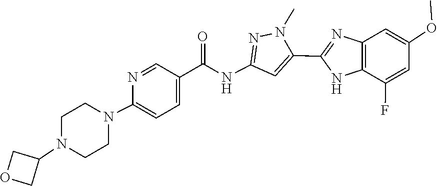 [SMILES: COc1cc(F)c2[nH]c(-c3cc(NC(=O)c4ccc(N5CCN(C6COC6)CC5)nc4)nn3C)nc2c1]

DIEA (0.0226 mL, 0.130 mmol) was added to a solution of 1-(oxetan-3-yl)piperazine (13.8 mg, 0.0973 mmol) and 6-chloro-N-[5-(7-fluoro-5-methoxy-1H-benzimidazol-2-yl)-1-methyl-pyrazol-3-yl]pyridine-3-carboxamide (L-14) (26 mg, 0.0649 mmol) in DMF (1 mL). The mixture was heated at 80° C. for 72 h. The mixture was cooled to 23° C., and water (10 mL) was added. The aq. phase was extracted with EtOAc (3×10 mL). The combined organic phases were washed with sat. NaHCO₃ (10 mL) and brine (10 mL), dried (MgSO₄), filtered, and concentrated. The residue was purified by silica gel chromatography (7 g cartridge) eluting with DCM and MeOH (5-10%) to provide the title compound as a solid (17 mg, 52%). ¹H NMR (400 MHz, DMSO) δ 13.19 (s, 1H), 10.84 (s, 1H), 8.80 (d, J=2.9 Hz, 1H), 8.15 (dd, J=9.1, 2.5 Hz, 1H), 7.45 (s, 1H), 6.90 (d, J=9.0 Hz, 1H), 6.83 (s, 1H), 6.75 (s, 1H), 4.57 (t, J=6.5 Hz, 2H), 4.48 (t, J=6.1 Hz, 2H), 4.27 (s, 3H), 3.83 (s, 3H), 3.70-3.61 (m, 4H), 3.44 (t, J=6.3 Hz, 1H), 2.39-2.30 (m, 4H). m/z (ES⁺), [M+H]⁺ 508.2. HPLC (A05) t_{R}=2.06 min.

### Preparation of Compound A-215 (N-[5-(1H-benzimidazol-2-yl)-1-methyl-pyrazol-3-yl]-4-[4-(oxetan-3-yl)piperazin-1-yl]benzamide)

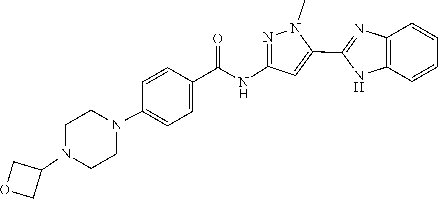 [SMILES: Cn1nc(NC(=O)c2ccc(N3CCN(C4COC4)CC3)cc2)cc1-c1nc2ccccc2[nH]1]

1-(Oxetan-3-yl)piperazine (0.212 g, 1.49 mmol) was added to a solution of N-[5-(1H-benzimidazol-2-yl)-1-methyl-pyrazol-3-yl]-4-fluoro-benzamide (L-16) (50 mg, 0.149 mmol) in DMSO (0.500 mL). The mixture was heated to 110° C. for 18 h. After cooling to 23° C., the mixture was concentrated. The residue was purified by silica gel chromatography (12 g cartridge) eluting with DCM and MeOH (0-10%) followed by a preparative HPLC purification (BEH C18 30×100) eluting with water (10 mM (NH₄)(HCO₃)) and MeCN (28-38%) to provide the title compound as a solid (25.5 mg, 37%). ¹H NMR (400 MHz, DMSO) δ 13.01 (s, 1H), 10.68 (s, 1H), 7.92 (d, J=9.0 Hz, 2H), 7.72-7.62 (m, 1H), 7.54-7.46 (m, 1H), 7.44 (s, 1H), 7.26-7.15 (m, 2H), 6.95 (d, J=9.1 Hz, 2H), 4.53 (t, J=6.5 Hz, 2H), 4.44 (t, J=6.1 Hz, 2H), 4.26 (s, 3H), 3.41 (p, J=6.3 Hz, 1H), 3.30-3.25 (m, 4H), 2.40-2.32 (m, 4H). m/z (ES⁺), [M+H]⁺ 458.8. HPLC (A05) t_{R}=2.02 min.

### Preparation of Compound A-216 (N-[5-(1H-benzimidazol-2-yl)-1-methyl-pyrazol-3-yl]-6-piperazin-1-yl-pyridine-3-carboxamide)

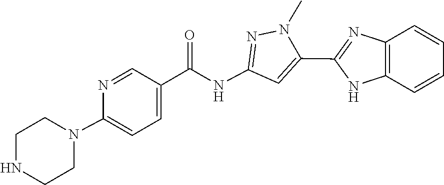 [SMILES: Cn1nc(NC(=O)c2ccc(N3CCNCC3)nc2)cc1-c1nc2ccccc2[nH]1]

DIEA (0.0247 mL, 0.142 mmol) was added to a solution of piperazine (8.25 μL, 0.00916 mmol) and N-[5-(1H-benzimidazol-2-yl)-1-methyl-pyrazol-3-yl]-6-chloro-pyridine-3-carboxamide (L-11) (25 mg, 0.0709 mmol) in DMF (0.500 mL). The mixture was heated at 80° C. for 66 h. The mixture was cooled to 23° C., and water (10 mL) was added. The aq. phase was extracted with EtOAc (3×10 mL). The combined organic phases were washed with brine (30 mL), dried (MgSO₄), filtered, and concentrated. The residue was purified by silica gel chromatography (7 g cartridge) eluting with DCM and MeOH (0-10%) to provide the title compound as a solid (10 mg, 35%). ¹H NMR (500 MHz, DMSO) δ 13.05 (s, 1H), 10.81 (s, 1H), 8.79 (d, J=2.4 Hz, 1H), 8.14 (dd, J=9.0, 2.5 Hz, 1H), 7.71 (d, J=7.6 Hz, 1H), 7.53 (d, J=7.9 Hz, 1H), 7.47 (s, 1H), 7.30-7.20 (m, 2H), 6.85 (d, J=8.8 Hz, 1H), 4.30 (s, 3H), 3.59-3.54 (m, 4H), 2.79 (s, 4H). m/z (ES⁺), [M+H]⁺ 404.1. HPLC (A05) t_{R}=1.82 min.

### Preparation of Compound A-217 (N-[5-(1H-benzimidazol-2-yl)-1-methyl-pyrazol-3-yl]-4-piperazin-1-yl-benzamide)

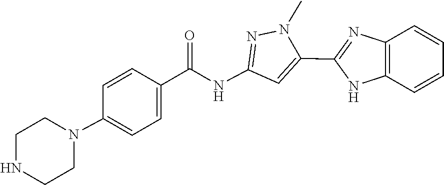 [SMILES: Cn1nc(NC(=O)c2ccc(N3CCNCC3)cc2)cc1-c1nc2ccccc2[nH]1]

N-[5-(1H-Benzimidazol-2-yl)-1-methyl-pyrazol-3-yl]-4-fluoro-benzamide (L-16) (28 mg, 0.0835 mmol) was suspended in 0.100 mL DMSO. Piperazine (71.9 mg, 0.835 mmol) was added, and the mixture was heated at 120° C. for 30 h. Water (50 mL) was added, and the aq. phase was extracted with EtOAc (3×15 mL). The combined organic phases were washed with brine (50 mL), dried (MgSO₄), filtered, and concentrated. The residue was purified by silica gel chromatography (12 g cartridge) eluting with DCM and MeOH (0-15%) to provide the title compound as a solid (11 mg, 33%). ¹H NMR (400 MHz, DMSO) δ 13.08 (s, 1H), 10.73 (s, 1H), 7.95 (d, J=9.0 Hz, 2H), 7.71 (s, 1H), 7.53 (d, J=8.1 Hz, 1H), 7.48 (s, 1H), 7.29-7.20 (m, 2H), 6.97 (d, J=9.1 Hz, 2H), 4.30 (s, 3H), 3.21 (s, 4H), 2.84 (s, 4H). m/z (ES⁺), [M+H]⁺ 402.5. HPLC (A05) t_{R}=1.87 min.

### Preparation of Compound A-219 (N-[5-(7-fluoro-5-methoxy-1H-benzimidazol-2-yl)-1-methyl-pyrazol-3-yl]-4-[4-(oxetan-3-yl)piperazin-1-yl]benzamide)

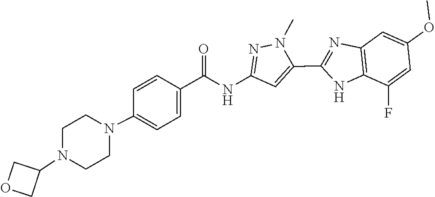 [SMILES: COc1cc(F)c2[nH]c(-c3cc(NC(=O)c4ccc(N5CCN(C6COC6)CC5)cc4)nn3C)nc2c1]

1-(Oxetan-3-yl)piperazine (0.00825 mL, 1.42 mmol) was added to a solution of 4-fluoro-N-[5-(7-fluoro-5-methoxy-1H-benzimidazol-2-yl)-1-methyl-pyrazol-3-yl]benzamide (L-17) (27 mg, 0.0709 mmol) in DMSO (0.500 mL). The mixture was heated at 120° C. for 30 h. The mixture was cooled to 23° C., and water (10 mL) was added. The aq. phase was extracted with EtOAc (3×10 mL). The combined organic phases were washed with brine (30 mL), dried (MgSO₄), filtered, and concentrated. The residue was purified by silica gel chromatography (12 g cartridge) eluting with DCM and MeOH (0-15%) to provide the title compound as a solid (13 mg, 36%). ¹H NMR (400 MHz, DMSO) δ 13.22 (s, 1H), 10.75 (s, 1H), 7.96 (d, J=9.0 Hz, 2H), 7.46 (s, 1H), 7.00 (d, J=9.1 Hz, 2H), 6.83 (s, 1H), 6.74 (d, J=11.9 Hz, 1H), 4.57 (t, J=6.5 Hz, 2H), 4.48 (t, J=6.1 Hz, 2H), 4.27 (s, 3H), 3.83 (s, 3H), 3.48-3.40 (m, 1H), 3.34-3.30 (m, 4H), 2.42-2.37 (m, 4H). ¹⁹F NMR (376 MHz, DMSO) δ −127.06 (s). m/z (ES⁺), [M+H]⁺ 506.4. HPLC (A05) t_{R}=2.13 min.

### Preparation of Compound A-221 (N-[5-(1H-benzimidazol-2-yl)-1-methyl-pyrazol-3-yl]-6-(3-oxopiperazin-1-yl)pyridine-3-carboxamide)

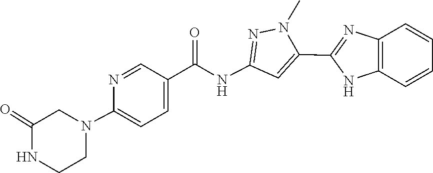 [SMILES: Cn1nc(NC(=O)c2ccc(N3CCNC(=O)C3)nc2)cc1-c1nc2ccccc2[nH]1]

DIEA (0.0494 mL, 0.283 mmol) was added to a solution of N-[5-(1H-benzimidazol-2-yl)-1-methyl-pyrazol-3-yl]-6-chloro-pyridine-3-carboxamide (L-11) (50 mg, 0.142 mmol) and piperazin-2-one (22.7 mg, 0.226 mmol) in DMF (1 mL). The mixture was heated at 90° C. for 18 h. The mixture was cooled to 23° C., and water (100 mL) was added. The aq. phase was extracted with EtOAc (3×15 mL). The combined organic phases were washed with brine (30 mL), dried (MgSO₄), filtered, and concentrated. The residue was purified by preparative HPLC (BEH C18 30×150) eluting with water (10 mM and (NH₄)(HCO₂)) and MeCN (24-34%) and by silica gel chromatography eluting with DCM and MeOH (0-25%) to provide the title compound as a solid (7.78 mg, 13.2%). ¹H NMR (400 MHz, DMSO) δ 13.06 (s, 1H), 10.87 (s, 1H), 8.83 (d, J=2.4 Hz, 1H), 8.19 (dd, J=9.0, 2.5 Hz, 1H), 8.16 (s, 1H), 7.71 (s, 1H), 7.54 (s, 1H), 7.48 (s, 1H), 7.25 (s, 2H), 6.87 (d, J=9.0 Hz, 1H), 4.30 (s, 3H), 4.12 (s, 2H), 3.87-3.80 (m, 2H), 3.17 (d, J=5.1 Hz, 2H). m/z (ES⁺), [M+H]⁺ 417.6. HPLC (A05) t_{R}=1.95 min.

### Preparation of Compound A-222 (N-[5-(7-fluoro-5-methoxy-1H-benzimidazol-2-yl)-1-methyl-pyrazol-3-yl]-4-[4-(2-hydroxyethyl)piperazin-1-yl]benzamide)

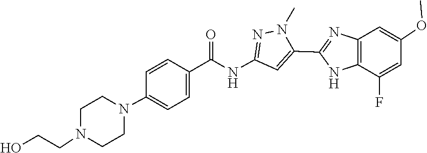 [SMILES: COc1cc(F)c2[nH]c(-c3cc(NC(=O)c4ccc(N5CCN(CCO)CC5)cc4)nn3C)nc2c1]

2-Piperazin-1-ylethanol (0.320 mL, 2.61 mmol) was added to a solution of 4-fluoro-N-[5-(7-fluoro-5-methoxy-1H-benzimidazol-2-yl)-1-methyl-pyrazol-3-yl]benzamide (L-17) (50 mg, 0.130 mmol) in DMSO (0.500 mL). The mixture was heated at 120° C. for 66 h and cooled to 23° C. Water (50 mL) was added, and the organic phases were extracted with EtOAc (3×15 mL). The combined organic phases were washed with brine (50 mL), dried (MgSO₄), filtered, and concentrated. The residue was purified by preparative HPLC (BEH C18 30×150) eluting with water (10 mM (NH₄)(HCO₃)) and MeCN (35-45%) and by silica gel chromatography (16 g cartridge) eluting with DCM and MeCN (0-100%) to provide the title compound as a solid (7.46 mg, 12%). ¹H NMR (400 MHz, DMSO) δ 13.19 (s, 1H), 10.72 (s, 1H), 7.96 (d, J=9.0 Hz, 2H), 7.46 (s, 1H), 6.99 (d, J=9.1 Hz, 2H), 6.84 (s, 1H), 6.75 (dd, J=12.3 Hz, 1H), 4.44 (t, J=5.3 Hz, 1H), 4.28 (s, 3H), 3.83 (s, 3H), 3.55 (dd, J=11.7, 6.2 Hz, 2H), 3.32-3.26 (m, 4H), 2.59-2.54 (m, 4H), 2.45 (t, J=6.2 Hz, 2H). ¹⁹F NMR (376 MHz, DMSO) δ −126.06 (s). m/z (ES⁺), [M+H]⁺ 494.6. HPLC (A05) t_{R}=1.92 min.

### Preparation of Compound A-224 (N-[5-(1H-benzimidazol-2-yl)-1-methyl-pyrazol-3-yl]-6-[2-(hydroxy-methyl)morpholin-4-yl]pyridine-3-carboxamide)

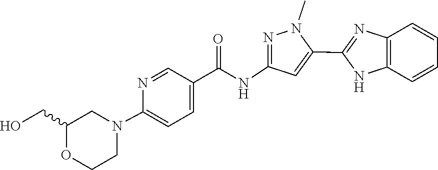 [SMILES: Cn1nc(NC(=O)c2ccc(N3CCOC(CO)C3)nc2)cc1-c1nc2ccccc2[nH]1]

Morpholin-2-ylmethanol (26.6 mg, 0.227 mmol) was added to a solution of N-[5-(1H-benzimidazol-2-yl)-1-methyl-pyrazol-3-yl]-6-chloro-pyridine-3-carboxamide (L-11) (80 mg, 0.227 mmol) and DIEA (0.0776 mL, 0.454 mmol) in DMSO (1.50 mL). The mixture was heated at 100° C. for 18 h. EtOAc (100 mL) was added, and the organic phase was washed with water (3×40 mL) and brine (50 mL), dried (MgSO₄), filtered, and concentrated. The residue was purified by silica gel chromatography (12 g cartridge) eluting with DCM and MeOH (0-50%) to provide the title compound as a solid (56.6 mg, 58%). ¹H NMR (400 MHz, DMSO) δ 13.06 (s, 1H), 10.86 (s, 1H), 8.82 (d, J=2.3 Hz, 1H), 8.18 (dd, J=9.1, 2.5 Hz, 1H), 7.72 (d, J=7.7 Hz, 1H), 7.53 (d, J=7.1 Hz, 1H), 7.47 (s, 1H), 7.29-7.19 (m, 2H), 6.89 (d, J=9.1 Hz, 1H), 4.82 (t, J=5.2 Hz, 1H), 4.37 (d, J=12.9 Hz, 1H), 4.30 (s, 3H), 4.18 (d, J=11.3 Hz, 1H), 3.95 (d, J=11.4 Hz, 1H), 3.59-3.43 (m, 4H), 3.01-2.91 (m, 1H), 2.75-2.66 (m, 1H). m/z (ES⁺), [M+H]⁺ 434.7. HPLC (A05) t_{R}=2.01 min.

### Preparation of Compound A-225 (N-[5-(1H-benzimidazol-2-yl)-1-methyl-pyrazol-3-yl]-6-[(3R)-3-hydroxy-pyrrolidin-1-yl]pyridine-3-carboxamide)

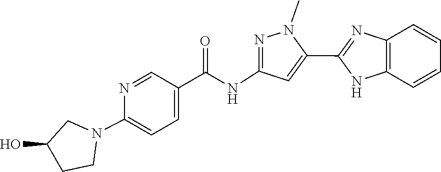 [SMILES: Cn1nc(NC(=O)c2ccc(N3CC[C@@H](O)C3)nc2)cc1-c1nc2ccccc2[nH]1]

DIEA (0.0494 mL, 0.283 mmol) was added to a solution of N-[5-(1H-benzimidazol-2-yl)-1-methyl-pyrazol-3-yl]-6-chloro-pyridine-3-carboxamide (L-11) (50 mg, 0.142 mmol) and (3R)-pyrrolidin-3-ol (19.8 mg, 0.227 mmol) in DMF (1 mL). The mixture was heated at 90° C. for 18 h. The mixture was cooled to 23° C. Water (10 mL) and EtOAc (10 mL) were added. The mixture was filtered, and the solid was purified by preparative HPLC (BEH C18 30×100) eluting with water (10 mM (NH₄)(HCO₂)) and MeCN (19-29%) to provide the title compound as a solid (9.60 mg, 17%). ¹EINMR (400 MHz, DMSO) δ 10.76 (s, 1H), 8.79 (d, J=2.5 Hz, 1H), 8.33 (s, 1H), 8.12 (dd, J=8.9, 2.5 Hz, 1H), 7.62 (s, 2H), 7.47 (s, 1H), 7.25 (dd, J=6.0, 3.0 Hz, 2H), 6.49 (d, J=8.8 Hz, 1H), 5.20-4.85 (m, 1H), 4.41 (s, 1H), 4.30 (s, 3H), 3.54 (dd, J=11.3, 4.3 Hz, 4H), 2.10-1.99 (m, 1H), 1.93 (s, 1H). m/z (ES⁺), [M+H]⁺ 404.3. HPLC (A05) t_{R}=1.96 min.

### Preparation of Compound A-226 (6-(4-acetylpiperazin-1-yl)-N-[5-(1H-benzimidazol-2-yl)-1-methyl-pyrazol-3-yl]pyridine-3-carboxamide)

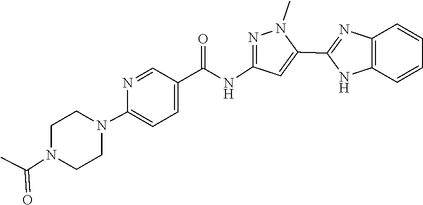 [SMILES: CC(=O)N1CCN(c2ccc(C(=O)Nc3cc(-c4nc5ccccc5[nH]4)n(C)n3)cn2)CC1]

1-Piperazin-1-ylethanone (29.1 mg, 0.227 mmol) was added to a solution of N-[5-(1H-benzimidazol-2-yl)-1-methyl-pyrazol-3-yl]-6-chloro-pyridine-3-carboxamide (L-11) (80 mg, 0.227 mmol) and DIEA (0.0776 mL, 0.454 mmol) in DMSO (1.50 mL). The mixture was heated at 100° C. for 18 h. EtOAc (60 mL) was added, and the organic phase was washed with water (3×40 mL) and brine (50 mL), dried (MgSO₄), filtered, and concentrated. The residue was purified by silica gel chromatography (12 g cartridge) eluting with DCM and MeOH (0-10%) to provide the title compound as a solid (43.5 mg, 43%). ¹H NMR (400 MHz, dmso) δ 13.06 (s, 1H), 10.86 (s, 1H), 8.82 (d, J=2.5 Hz, 1H), 8.18 (dd, J=9.1, 2.5 Hz, 1H), 7.75-7.63 (m, 1H), 7.60-7.49 (m, 1H), 7.47 (s, 1H), 7.30-7.21 (m, 2H), 6.91 (d, J=9.1 Hz, 1H), 4.30 (s, 3H), 3.71 (dd, J=6.9, 3.6 Hz, 2H), 3.63 (dd, J=6.6, 3.4 Hz, 2H), 3.56 (dd, J=6.6, 3.8 Hz, 4H), 2.06 (s, 3H). m/z (ES⁺), [M+H]⁺ 445.4. HPLC (A05) t_{R}=2.02 min.

### Preparation of Compound A-227 (N-[5-(1H-benzimidazol-2-yl)-1-methyl-pyrazol-3-yl]-6-[(3R)-3-hydroxy-pyrrolidin-1-yl]pyridine-3-carboxamide)

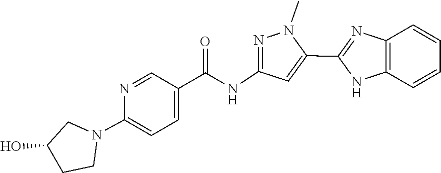 [SMILES: Cn1nc(NC(=O)c2ccc(N3CC[C@H](O)C3)nc2)cc1-c1nc2ccccc2[nH]1]

DIEA (0.0494 mL, 0.283 mmol) was added to a solution of N-[5-(1H-benzimidazol-2-yl)-1-methyl-pyrazol-3-yl]-6-chloro-pyridine-3-carboxamide (L-11) (50 mg, 0.142 mmol) and (3S)-pyrrolidin-3-ol (19.8 mg, 0.227 mmol) in DMF (1 mL). The mixture was heated at 90° C. for 18 h. The mixture was cooled to 23° C. and concentrated. The residue was purified by preparative HPLC (BEH C18 30×100) eluting with water (10 mM (NH₄)(HCO₃)) and MeCN (24-34%) to provide the title compound as a solid (45.2 mg, 79%). ¹H NMR (400 MHz, dmso) δ 13.13-12.97 (m, 1H), 10.76 (s, 1H), 8.79 (d, J=2.7 Hz, 1H), 8.12 (dd, J=8.9, 2.5 Hz, 1H), 7.77-7.63 (m, 1H), 7.63-7.48 (m, 1H), 7.47 (s, 1H), 7.24 (s, 2H), 6.49 (d, J=9.0 Hz, 1H), 5.00 (d, J=3.6 Hz, 1H), 4.41 (s, 1H), 4.30 (s, 3H), 3.53 (s, 3H), 3.45-3.34 (m, 1H), 2.03 (s, 1H), 1.97-1.86 (m, 1H). m/z (ES⁺), [M+H]⁺ 404.3. HPLC (A05) t_{R}=1.96 min.

### EXAMPLE B16

### Preparation of Compounds A-1 and A-5

### Representative Synthetic Scheme for the Syntheses of Compounds A-1 and A-5.

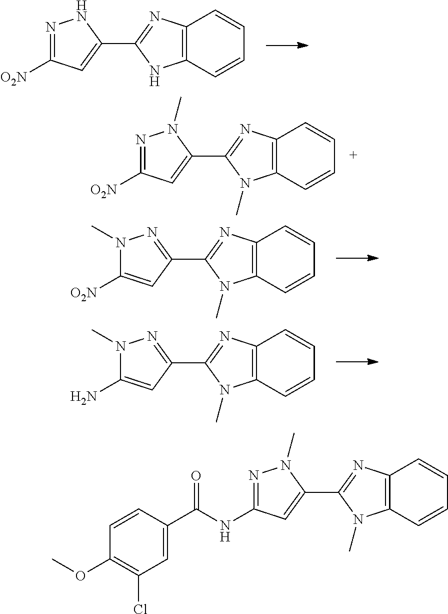

### Preparation of Compound A-1.

### Step 1: 1-methyl-2-(1- and 1-methyl-2-(2-methyl-5-nitro-pyrazol-3-yl)-benzimidazole)

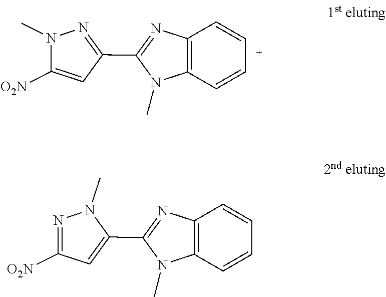 [SMILES: Cn1nc(-c2nc3ccccc3n2C)cc1[N+](=O)[O-] ; Cn1nc([N+](=O)[O-])cc1-c1nc2ccccc2n1C]

DMF (10 mL) was added to a mixture of 2-(5-nitro-1H-pyrazol-3-yl)-1H-benzimidazole (N-1) (0.250 g, 1.09 mmol) and Cs₂CO₃ (1.07 g, 3.27 mmol). MeI (0.204 mL, 3.27 mmol) was added, and the mixture was stirred at 23° C. for 2.5 h. The mixture was concentrated. Water (10 mL) was added, and the aq. phase was extracted with EtOAc (3×20 mL). The combined organic phases were washed with brine (10 mL), dried (MgSO₄), filtered, and concentrated. The residue was purified by silica gel chromatography (24 g cartridge) eluting with hexanes and EtOAc (0-30%) to provide two compounds. 1st eluting compound: 1-methyl-2-(1-methyl-5-nitro-pyrazol-3-yl)benzimidazole as a solid (85.8 mg, 31%). ¹H NMR (500 MHz, CDCl₃) δ 7.80-7.77 (m, 1H), 7.76 (s, 1H), 7.40-7.36 (m, 1H), 7.34-7.27 (m, 2H), 4.31 (s, 3H), 4.15 (s, 3H). ¹³C NMR (126 MHz, CDCl₃) δ 146.71, 144.77, 143.01, 142.83, 136.80, 123.77, 123.05, 120.22, 109.98, 107.40, 41.66, 32.34. m/z (ES⁺), [M+H]⁺ 257.4. HPLC (A05) t_{R}=2.16 min. 2nd eluting compound: 1-methyl-2-(2-methyl-5-nitro-pyrazol-3-yl)benzimidazole as a solid (94.9 mg, 34%). ¹H NMR (500 MHz, CDCl₃) δ 7.86-7.82 (m, 1H), 7.48-7.34 (m, 3H), 7.20 (s, 1H), 4.30 (s, 3H), 3.93 (s, 3H). ¹³C NMR (126 MHz, CDCl₃) δ 154.92, 142.84, 141.58, 135.95, 134.85, 124.77, 123.72, 120.83, 110.37, 104.17, 40.73, 31.76. m/z (ES⁺), [M+H]⁺ 257.4. HPLC (A05) t_{R}=2.09 min.

### Step 2: 1-methyl-5-(1-methylbenzimidazol-2-yl)pyrazol-3-amine

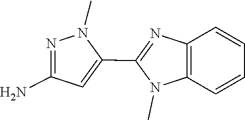 [SMILES: Cn1nc(N)cc1-c1nc2ccccc2n1C]

AcOH (2 mL) was added to a mixture of iron powder (109 mg, 1.94 mmol) and 1-methyl-2-(1-methyl-5-nitro-pyrazol-3-yl)benzimidazole (50 mg, 0.194 mmol). The mixture was stirred at 23° C. for 2 h. The reaction mixture was poured dropwise into sat. NaHCO₃ (20 mL). Rochelle salt (10 mL) was added. The aq. phase was extracted with EtOAc (3×25 mL). The combined organic phases were washed with brine (25 mL), dried (MgSO₄), filtered, and concentrated to provide the title compound as a solid (43 mg, 97%). ¹H NMR (500 MHz, DMSO) δ 7.73-7.70 (m, 1H), 7.65-7.62 (m, 1H), 7.35-7.31 (m, 1H), 7.29-7.25 (m, 1H), 5.97 (s, 1H), 4.81 (s, 2H), 3.87 (s, 3H), 3.85 (s, 3H). m/z (ES⁺), [M+H]⁺ 228.5. HPLC (A05) t_{R}=1.74 min.

### Step 3: 3-chloro-4-methoxy-N-[1-methyl-5-(1-methylbenzimidazol-2-yl)pyrazol-3-yl]benzamide

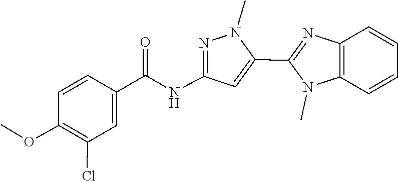 [SMILES: COc1ccc(C(=O)Nc2cc(-c3nc4ccccc4n3C)n(C)n2)cc1Cl]

3-Chloro-4-methoxy-benzoic acid (18.1 mg, 0.0968 mmol) and HATU (36.8 mg, 0.0968 mmol) were dissolved in DMF (0.500 mL) at 0° C. DIEA (33.1 μL, 0.194 mmol) was added, and the mixture was stirred at 0° C. for 5 min. A solution of 1-methyl-5-(1-methylbenzimidazol-2-yl)pyrazol-3-amine (22 mg, 0.0968 mmol) in DMF (0.500 mL) was added. The mixture was warmed to 23° C. and stirred for 18 h. The mixture was concentrated. Water (10 mL) was added, and the aq. phase was extracted with EtOAc (3×20 mL). The combined organic phases were washed with brine (20 mL), dried (MgSO₄), filtered, and concentrated. The product was purified by HPLC (BEH C₁₈ 30×100 mm) eluting with water (10 mM (NH₄)(HCO₂)) and MeCN (43-63%). The purified residue was dissolved in EtOAc (10 mL), and the mixture was washed with water (10 mL), dried (MgSO₄), filtered, and concentrated to provide the title compound as a solid (16.2 mg, 42%). ¹H NMR (500 MHz, DMSO) δ 11.07 (s, 1H), 8.18 (d, J=2.2 Hz, 1H), 8.08 (dd, J=8.7, 2.2 Hz, 1H), 7.79-7.74 (m, 1H), 7.71-7.66 (m, 1H), 7.39-7.35 (m, 1H), 7.33-7.29 (m, 1H), 7.30 (d, J=8.8 Hz, 1H), 7.19 (s, 1H), 4.07 (s, 3H), 3.96 (s, 3H), 3.94 (s, 3H). m/z (ES⁺), [M+H]⁺ 396.6. HPLC (A05) t_{R}=2.24 min.

### Preparation of Compound A-5.

### Step 1: 2-methyl-5-(1-methylbenzimidazol-2-yl)pyrazol-3-amine

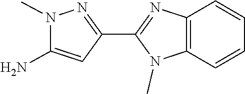 [SMILES: Cn1nc(-c2nc3ccccc3n2C)cc1N]

AcOH (1.50 mL) was added to a mixture of iron powder (76 mg, 1.36 mmol) and 1-methyl-2-(1-methyl-5-nitro-pyrazol-3-yl)benzimidazole (A-1, Step 1) (35 mg, 0.136 mmol). The mixture was stirred at 23° C. for 2 h. The mixture was poured dropwise into sat. NaHCO₃ (20 mL). Rochelle salt (10 mL) was added. The aq. phase was extracted with EtOAc (3×25 mL). The combined organic phases were washed with brine (25 mL), dried (MgSO₄), filtered, and concentrated to provide the title compound as a solid (27.5 mg, 89%). ¹H NMR (500 MHz, DMSO) δ 7.62-7.58 (m, 1H), 7.54-7.51 (m, 1H), 7.26-7.22 (m, 1H), 7.22-7.17 (m, 1H), 5.94 (s, 1H), 5.45 (s, 2H), 4.13 (s, 3H), 3.67 (s, 3H). m/z (ES⁺), [M+H]⁺ 228.5. HPLC (A05) t_{R}=1.63 min.

### Step 2: 3-chloro-4-methoxy-N-[2-methyl-5-(1-methylbenzimidazol-2-yl)pyrazol-3-yl]benzamide

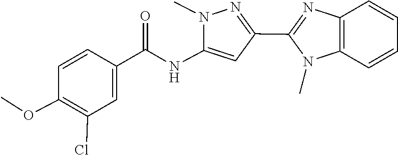 [SMILES: COc1ccc(C(=O)Nc2cc(-c3nc4ccccc4n3C)nn2C)cc1Cl]

3-Chloro-4-methoxy-benzoic acid (10.4 mg, 0.0559 mmol) and HATU (21.2 mg, 0.0559 mmol) were dissolved in DMF (0.500 mL) at 0° C. DIEA (19.1 μL, 0.112 mmol) was added, and the mixture was stirred at 0° C. for 5 min. A solution of 2-methyl-5-(1-methylbenzimidazol-2-yl)pyrazol-3-amine (12.7 mg, 0.0559 mmol) in DMF (0.500 mL) was added. The mixture was warmed to 23° C. and stirred for 18 h. A solution of 3-chloro-4-methoxy-benzoic acid (10.4 mg, 0.0559 mmol), HATU (21.2 mg, 0.0559 mmol), and DIEA (19.1 μL, 0.112 mmol) in DMF (0.500 mL) at 0° C. was added, and the mixture warmed to 50° C. and stirred for an additional 18 h. The mixture was concentrated. Brine (10 mL) was added, and the aq. phase was extracted with EtOAc (3×20 mL). The combined organic phases were washed with brine (20 mL), dried (MgSO₄), filtered, and concentrated. The product was purified by HPLC (BEH C18 30×150 mm) eluting with water (10 mM (NH₄)(HCO₂)) and MeCN (35-55%). The purified residue was dissolved in EtOAc (10 mL) and washed with water (10 mL). The organic phase was dried (MgSO₄), filtered, and concentrated to provide the title compound as a solid (2.38 mg, 11%). ¹H NMR (500 MHz, DMSO) δ 10.49 (s, 1H), 8.14 (d, J=2.2 Hz, 1H), 8.03 (dd, J=8.6, 2.2 Hz, 1H), 7.68-7.63 (m, 1H), 7.62-7.58 (m, 1H), 7.37-7.33 (m, 1H), 7.31-7.27 (m, 1H), 7.26-7.20 (m, 1H), 6.90 (s, 1H), 4.19 (s, 3H), 3.98 (s, 3H), 3.87 (s, 3H). m/z (ES⁺), [M+H]⁺ 396.5. HPLC (A05) t_{R}=2.17 min.

### EXAMPLE B17

### Preparation of Compounds A-2, A-3, A-7, A-8, and A-12

### Representative Synthetic Scheme for the Syntheses of the Title Compounds.

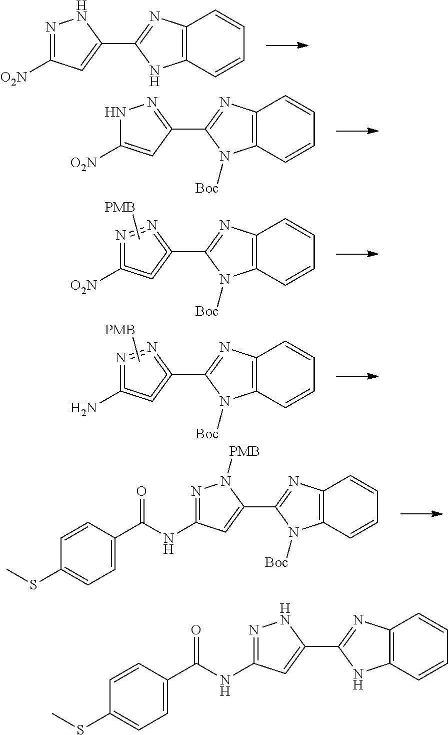

### Preparation of Compound A-2.

### Step 1: tert-butyl 2-(5-nitro-1H-pyrazol-3-yl)benzimidazole-1-carboxylate

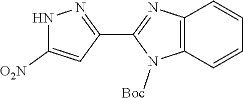 [SMILES: CC(C)(C)OC(=O)n1c(-c2cc([N+](=O)[O-])[nH]n2)nc2ccccc21]

2-(5-Nitro-1H-pyrazol-3-yl)-1H-benzimidazole (1.00 g, 4.36 mmol) and DMAP (53.3 mg, 0.436 mmol) were dissolved in DMF (20 mL), and cooled to 0° C. NEt₃ (1.82 mL, 13.1 mmol) and a solution of Boc₂O (2.38 g, 10.9 mmol) in DMF (5 mL) were added. The mixture was stirred at 0° C. for 5 min, warmed to 23° C., and stirred for 6 h. The mixture was concentrated. Water (50 mL) was added, and the aq. phase was extracted with EtOAc (3×50 mL). The combined organic phases were washed with brine (50 mL), dried (MgSO₄), filtered, and concentrated. The residue was purified by silica gel chromatography (40 g cartridge) eluting with hexanes and EtOAc (0-100%) to provide the title compound as a solid (806 mg; 56%). ¹H NMR (500 MHz, DMSO) δ 14.82 (s, 1H), 8.09-8.03 (m, J=8.1 Hz, 1H), 7.88-7.83 (m, J=7.7 Hz, 1H), 7.58 (s, 1H), 7.57-7.52 (m, 1H), 7.50-7.45 (m, 1H), 1.54 (s, 9H). m/z (ES⁺), [M+H−Boc]⁺ 230.3. HPLC (A05) t_{R}=2.35 min.

### Step 2: tert-butyl 2-[2- and tert-butyl 2-[1-[(4-methoxyphenyl)methyl]-5-nitro-pyrazol-3-yl]benzimidazole-1-carboxylate

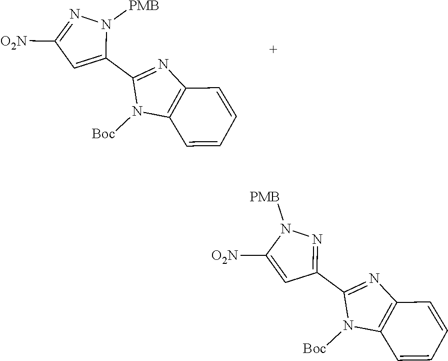

DMF (20 mL) was added to a mixture of tert-butyl 2-(5-nitro-1H-pyrazol-3-yl)benzimidazole-1-carboxylate (0.806 g, 2.45 mmol) and Cs₂CO₃ (0.957 g, 2.94 mmol). The mixture was stirred at 23° C. for 10 min. 4-Methoxybenzyl chloride (0.398 mL, 2.94 mmol) was added, and the mixture was stirred at 23° C. for 18 h. The mixture was concentrated. Water (25 mL) was added, and the aq. phase was extracted with EtOAc (3×25 mL). The combined organic phases were washed with brine (25 mL), dried (MgSO₄), filtered and concentrated. The product was purified by silica gel chromatography (80 g cartridge) eluting with hexanes and EtOAc (0-30%) to provide a 3:1 mixture of pyrazole isomers as a solid (0.780 g, 71%). ¹H NMR (500 MHz, CDCl₃) δ 8.06-8.02 [8.00-7.96] (m, 1H), 7.87-7.82 [7.87-7.82] (m, 1H), 7.52-7.37 (m, 2H), 7.12 [7.49] (s, 1H), 6.94-6.88 [7.35-7.30] (m, 2H), 6.65-6.58 [6.87-6.81] (m, 2H), 5.45 [5.82] (s, 2H), 3.68 [3.78] (s, 3H), 1.39 [1.57] (s, 9H). m/z (ES+), [M+H−Boc]⁺ 350.6. HPLC (A05) t_{R}=2.57 [2.64] min.

### Step 3: tert-butyl 2-[5-amino-2- and tert-butyl 2-15-amino-1-[(4-methoxy-phenyl)methyl]pyrazol-3-yl]benzimidazole-1-carboxylate

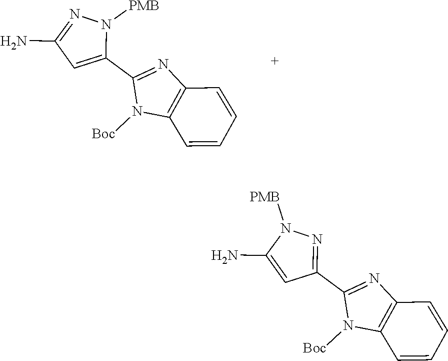

A 3:1 mixture of tert-butyl 2-[2- and tert-butyl 241-[(4-methoxyphenyl)methyl]-5-nitro-pyrazol-3-yl]benzimidazole-1-carboxylate (199 mg, 0.443 mmol) was dissolved in AcOH (5 mL). Iron powder (247 mg, 4.43 mmol) was added, and the mixture was stirred at 23° C. for 4 h. The mixture was filtered through Celite, washing with EtOAc. The filtrate was concentrated. Sat. NaHCO₃ (20 mL) and Rochelle salt (20 mL) were added to the residue. The aq. phase was extracted with EtOAc (3×30 mL). The combined organic phases were washed with sat. NaHCO₃ (30 mL), Rochelle salt (30 mL), and brine (30 mL). The organic phases were dried (MgSO₄), filtered, and concentrated. The residue was purified by silica gel chromatography (25 g cartridge) eluting with DCM and MeOH (0-5%) to provide a 3:1 mixture of pyrazole isomers as a solid (136 mg, 73%). ¹H NMR (500 MHz, DMSO) δ 8.03-7.99 [7.83-7.78] (m, 1H), 7.83-7.78 [7.72-7.68] (m, 1H), 7.50-7.45 [7.40-7.37] (m, 1H), 7.45-7.40 [7.37-7.32] (m, 1H), 7.02-6.98 [7.25-7.20] (m, 2H), 6.79-6.74 [6.92-6.86] (m, 2H), 5.80 [5.74] (s, 1H), 5.03 [5.52] (s, 2H), 4.77 [5.14] (s, 2H), 3.67 [3.73] (s, 3H), 1.42 [1.42] (s, 9H). m/z (ES+), [M+H]⁺ 420.1. HPLC (A05) t_{R}=2.29 [2.34] min.

### Step 4: tert-butyl 2-[2-[(4-methoxyphenyl)methyl]-5-[(4-methylsulfanyl-benzoyl)amino]pyrazol-3-yl]benzimidazole-1-carboxylate

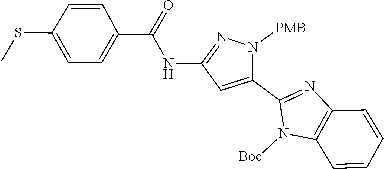

4-(Methylthio)benzoic acid (20.1 mg, 0.119 mmol) and HATU (45.3 mg, 0.119 mmol) were dissolved in DMF (1 mL) at 0° C. DIEA (40.8 μL, 0.228 mmol) was added, and the mixture was stirred at 0° C. for 5 min. A 3:1 mixture of tert-butyl 2-[5-amino-2- and tert-butyl 2-[5-amino-1-[(4-methoxyphenyl)methyl]pyrazol-3-yl]benzimidazole-1-carboxylate (50 mg, 0.119 mmol) was added. The mixture was warmed to 50° C. and stirred for 18 h. The mixture was concentrated. Brine (10 mL) was added, and the aq. phase was extracted with EtOAc (3×20 mL). The combined organic phases were washed with brine (20 mL), dried (MgSO₄), filtered, and concentrated. The product was purified by silica gel chromatography (12 g cartridge) eluting with hexanes and EtOAc (0-100%) to provide the title compound as a solid (34.1 mg; 50%). ¹H NMR (500 MHz, DMSO) δ 10.98 (s, 1H), 8.09-8.04 (m, 1H), 8.03-7.96 (m, 2H), 7.89-7.83 (m, 1H), 7.55-7.50 (m, 1H), 7.49-7.44 (m, 1H), 7.37-7.32 (m, 2H), 7.08-7.04 (m, 2H), 7.02 (s, 1H), 6.83-6.77 (m, 2H), 5.27 (s, 2H), 3.68 (s, 3H), 2.54 (s, 3H), 1.41 (s, 9H). m/z (ES⁺), [M+H]⁺ 570.4. HPLC (A05) t_{R}=2.63 min.

### Step 5: N-[5-(1H-benzimidazol-2-yl)-1H-pyrazol-3-yl]-4-methylsulfanyl-benzamide

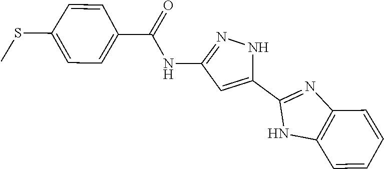 [SMILES: CSc1ccc(C(=O)Nc2cc(-c3nc4ccccc4[nH]3)[nH]n2)cc1]

tert-Butyl 2-[2-[(4-methoxyphenyl)methyl]-5-[(4-methylsulfanylbenzoyl)amino]pyrazol-3-yl]-benzimidazole-1-carboxylate (32.3 mg, 0.0567 mmol) was dissolved in DCM (0.500 mL). TFA (0.500 mL) was added, and the mixture was stirred at 23° C. for 1.5 h. The mixture was concentrated. The residue was diluted in sat. NaHCO₃ (10 mL) and stirred for 5 min. The mixture was filtered, and the solid was washed with water, EtOAc, DCM, MeCN, and hexanes. The organic phases were dried under high vacuum to provide a 3:1 mixture of pyrazole tautomers of the title compound as a solid (13.4 mg, 68%). ¹H NMR (500 MHz, DMSO) δ 13.50 [13.17] (s, 1H), 13.03 [12.74] (s, 1H), 10.96 [11.14] (s, 1H), 8.09-7.91 [8.09-7.91] (m, 2H), 7.71-7.65 [7.65-7.59] (m, 1H), 7.44 [6.68] (s, 1H), 7.58-7.32 [7.58-7.32] (m, 3H), 7.28-7.21 [7.21-7.12] (m, 2H), 2.56 [2.56] (s, 3H). m/z (ES⁺), [M−H]⁻ 348.1. HPLC (A05) t_{R}=2.10 min.

### Preparation of Compound A-3.

### Step 1: tert-butyl 2-[2-[(4-methoxyphenyl)methyl]-5-[(4-methylsulfanyl-benzoyl)-amino]pyrazol-3-yl]benzimidazole-1-carboxylate

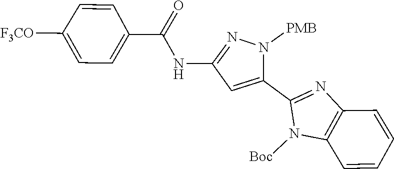

4-(Trifluoromethoxy)benzoic acid (24.6 mg, 0.119 mmol) and HATU (45.3 mg, 0.119 mmol) were dissolved in DMF (1 mL) at 0° C. DIEA (40.8 μL, 0.228 mmol) was added, and the mixture was stirred at 0° C. for 5 min. A 3:1 mixture of tert-butyl 2-[5-amino-2- and tert-butyl 2-[5-amino-1-[(4-methoxyphenyl)methyl]pyrazol-3-yl]benzimidazole-1-carboxylate (A-2, Step 3) (50 mg, 0.119 mmol) was added. The mixture was warmed to 23° C. and stirred for 18 h. The mixture was concentrated. Brine (10 mL) was added, and the aq. phase was extracted with EtOAc (3×20 mL). The combined organic phases were washed with brine (20 mL), dried (MgSO₄), filtered, and concentrated. The product was purified by silica gel chromatography (12 g cartridge) eluting with hexanes and EtOAc (0-75%) to provide the title compound as a solid (44.2 mg; 61%). ¹H NMR (500 MHz, DMSO) δ 11.19 (s, 1H), 8.20-8.13 (m, 2H), 8.09-8.04 (m, 1H), 7.86 (d, J=7.6 Hz, 1H), 7.55-7.43 (m, 4H), 7.08-7.04 (m, 2H), 7.03 (s, 1H), 6.83-6.77 (m, 2H), 5.27 (s, 2H), 3.68 (s, 3H), 1.40 (s, 9H). m/z (ES⁺), [M+H]⁺ 607.4. HPLC (A05) t_{R}=2.70 min.

### Step 2: N-[5-(1H-benzimidazol-2-yl)-1H-pyrazol-3-yl]-4-(trifluoro-methoxy)-benzamide

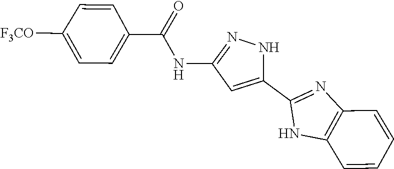 [SMILES: O=C(Nc1cc(-c2nc3ccccc3[nH]2)[nH]n1)c1ccc(OC(F)(F)F)cc1]

tert-Butyl 2-[2-[(4-methoxyphenyl)methyl]-5-[[4-(trifluoromethoxy)benzoyl]amino]pyrazol-3-yl]benzimidazole-1-carboxylate (38.5 mg, 0.0634 mmol) was dissolved in DCM (0.500 mL). TFA (0.500 mL) was added, and the mixture was stirred at 23° C. for 1.5 h. The mixture was concentrated. The residue was diluted in sat. NaHCO₃ (10 mL) and stirred for 5 min. The mixture was filtered, and the solid was washed with water, EtOAc, DCM, MeCN. and hexanes. The solid was dried under high vacuum to provide the title compound as a solid (11.4 mg, 46%). ¹H NMR (500 MHz, DMSO) δ 13.37 (br, 2H), 11.24 (br, 1H), 8.28-8.04 (m, 2H), 7.71-7.60 (m, 2H), 7.60-7.48 (m, 2H), 7.40-7.18 (m, 3H). ¹⁹F NMR (471 MHz, DMSO) δ −56.60. m/z (ES), [M−H]⁻ 386.1. HPLC (A05) t_{R}=2.21 min.

### Preparation of Compound A-7.

### Step 1: tert-butyl 2-[2-[(4-methoxyphenyl)methyl]-5-[(2-methoxypyrimidine-5-carbonyl)amino]pyrazol-3-yl]benzimidazole-1-carboxylate

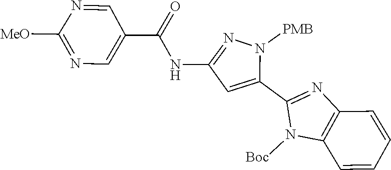

2-Methoxypyrimidine-5-carboxylic acid (18.4 mg, 0.119 mmol) and HATU (45.3 mg, 0.119 mmol) were dissolved in DMF (1 mL) at 0° C. DIEA (40.8 μL, 0.238 mmol) was added, and the mixture was stirred at 0° C. for 5 min. A 3:1 mixture of tert-butyl 2-[5-amino-2- and tert-butyl 2-[5-amino-1-[(4-methoxyphenyl)methyl]pyrazol-3-yl]benzimidazole-1-carboxylate (A-2, Step 3) (50 mg, 0.119 mmol) was added. The mixture was warmed to 50° C. and stirred for 18 h. The mixture was concentrated. Water (10 mL) was added, and the aq. phase was extracted with EtOAc (3×20 mL). The combined organic phases were washed with brine (20 mL), dried (MgSO₄), filtered, and concentrated. The product was purified by silica gel chromatography (12 g cartridge) eluting with DCM and MeOH (0-10%) to provide the title compound as a solid (60 mg; 91%). ¹H NMR (500 MHz, DMSO) δ 11.28 (s, 1H), 9.15 (s, 2H), 8.06 (d, J=8.1 Hz, 1H), 7.85 (d, J=7.7 Hz, 1H), 7.53-7.50 (m, 1H), 7.48-7.44 (m, 1H), 7.06 (d, J=8.7 Hz, 2H), 7.01 (s, 1H), 6.80 (d, J=8.8 Hz, 2H), 5.26 (s, 2H), 4.00 (s, 3H), 3.67 (s, 3H), 1.40 (s, 9H). m/z (ES⁺), [M+H]⁺ 556.3. HPLC (A05) t_{R}=2.78 min.

### Step 2: N-[5-(1H-benzimidazol-2-yl)-1H-pyrazol-3-yl]-2-methoxy-pyrimidine-5-carboxamide

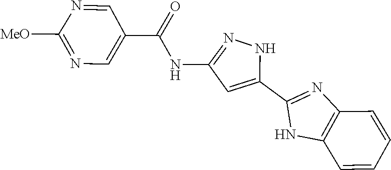 [SMILES: COc1ncc(C(=O)Nc2cc(-c3nc4ccccc4[nH]3)[nH]n2)cn1]

tert-Butyl 2-[2-[(4-methoxyphenyl)methyl]-5-[(2-methoxypyrimidine-5-carbonyl)amino]-pyrazol-3-yl]benzimidazole-1-carboxylate (60 mg, 0.108 mmol) was dissolved in DCM (2 mL) at 23° C. TFA (2 mL) was added, and the mixture was stirred for 18 h at 23° C. The mixture was concentrated. Sat. NaHCO₃ (15 mL) was added, and the mixture was filtered. The solid was washed with water (10 mL), MeCN (10 mL), EtOAc (10 mL), and hexanes (10 mL) and dried under high vacuum to provide the title compound as a solid (20 mg, 55%). ¹H NMR (500 MHz, DMSO) δ 13.57 (s, 1H), 13.05 (s, 1H), 11.27 (s, 1H), 9.16 (s, 2H), 7.71-7.49 (m, 2H), 7.44 (s, 1H), 7.29-7.18 (m, 2H), 4.01 (s, 3H). m/z (ES⁺), [M+H]⁺ 336.2. HPLC (A05) t_{R}=2.09 min.

### Preparation of Compound A-8.

### Step 1: tert-butyl 2-[2-[(4-methoxyphenyl)methyl]-5-[(6-methoxypyridine-3-carbonyl)amino]pyrazol-3-yl]benzimidazole-1-carboxylate

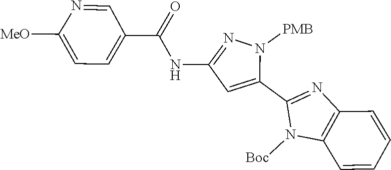

6-Methoxypyridine-3-carboxylic acid (28.6 mg, 0.187 mmol) and HATU (71.2 mg, 0.187 mmol) were dissolved in DMF (1.50 mL) at 0° C. DIEA (49.0 μL, 0.286 mmol) was added, and the mixture was stirred at 0° C. for 5 min. A 3:1 mixture of tert-butyl 2-[5-amino-2- and tert-butyl 245-amino-1-[(4-methoxyphenyl)methyl]pyrazol-3-yl]benzimidazole-1-carboxylate (A-2, Step 3) (60 mg, 0.119 mmol) was added. The mixture was warmed at 23° C. and stirred for 18 h. The mixture was concentrated. Brine (10 mL) was added, and the aq. phase was extracted with EtOAc (3×20 mL). The combined organic phases were washed with brine (20 mL), dried (MgSO₄), filtered, and concentrated. The product was purified by silica gel chromatography (4 g cartridge) eluting with hexanes and EtOAc (0-100%) to provide the title compound as a solid (58 mg; 73%). ¹H NMR (500 MHz, CDCl₃) δ 8.73 (d, J=2.5 Hz, 1H), 8.72 (s, 1H), 8.14-8.07 (m, 2H), 7.84 (dd, J=7.2, 1.3 Hz, 1H), 7.49-7.39 (m, 2H), 7.15 (s, 1H), 7.07-7.02 (m, 2H), 6.81 (d, J=8.7 Hz, 1H), 6.74-6.69 (m, 2H), 5.30 (s, 2H), 4.00 (s, 3H), 3.72 (s, 3H), 1.44 (s, 9H). m/z (ES⁺), [M+H]⁺ 555.5. HPLC (A05) t_{R}=2.52 min.

### Step 2: N-[5-(1H-benzimidazol-2-yl)-1H-pyrazol-3-yl]-6-methoxy-pyridine-3-carboxamide

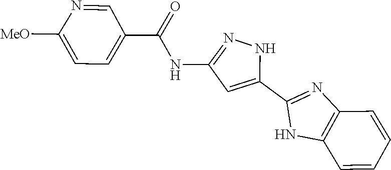 [SMILES: COc1ccc(C(=O)Nc2cc(-c3nc4ccccc4[nH]3)[nH]n2)cn1]

tert-Butyl 2-[2-[(4-methoxyphenyl)methyl]-5-[(4-methylsulfanylbenzoyl)amino]pyrazol-3-yl]-benzimidazole-1-carboxylate (32.3 mg, 0.0567 mmol) was dissolved in DCM (0.500 mL). TFA (0.500 mL) was added, and the mixture was stirred at 23° C. for 3 h. The mixture was concentrated. The residue was diluted in sat. aq. NaHCO₃ (10 mL) and stirred for 5 min. The mixture was filtered, and the solid was washed with water, MeCN, DCM, and hexanes. The solid was purified by HPLC (BEH C18 30×100 mm) eluting with water (10 mM (NH₄)(HCO₂)) and MeCN (29-49%). The purified residue was diluted in 1.0 M NaOH (5 mL). The mixture was filtered, and the solid was washed with water, MeCN, DCM, and hexanes and dried under high vacuum to provide a 5:1 mixture of pyrazole tautomers of the title compound as a solid (3.98 mg, 11%). ¹H NMR (500 MHz, DMSO) δ 13.51 (s, 1H) [13.17 (s, 1H)], 13.02 (s, 1H) [12.74 (s, 1H)], 11.06 (s, 1H) [11.17 (s, 1H)], 8.87 (d, J=2.2 Hz, 1H) [8.85-8.84 (m, 1H)], 8.31 (dd, J=8.7, 2.4 Hz, 1H) [8.29-8.26 (m, 1H)], 7.69-7.65 (m, 1H) [7.65-7.60 (m, 1H)], 7.54 (d, J=7.8 Hz, 1H) [7.48-7.44 (m, 1H)], 7.43 (s, 1H) [ 6.67 (s, 1H)], 7.29-7.19 (m, 2H) [7.19-7.14 (m, 2H)], 6.94 (d, J=8.8 Hz, 1H) [7.03-6.99 (m, 1H)], 3.94 (s, 3H) [3.96 (s, 3H)]. m/z (ES⁺), [M+H]⁺ 335.0. HPLC (A05) t_{R}=1.93 min.

### Preparation of Compound A-12.

### Step 1: tert-butyl 2-[2-[(4-methoxyphenyl)methyl]-5-[(5-methoxypyridine-2-carbonyl)amino]pyrazol-3-yl]benzimidazole-1-carboxylate

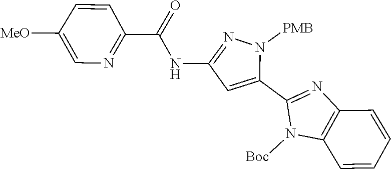

5-Methoxypyridine-2-carboxylic acid (33.4 mg, 0.218 mmol) and HATU (82.5 mg, 0.217 mmol) were dissolved in DMF (1.75 mL) at 0° C. DIEA (57.1 μL, 0.334 mmol) was added, and the mixture was stirred at 0° C. for 5 min. A 3:1 mixture of tert-butyl 2-[5-amino-2- and tert-butyl 2-[5-amino-1-[(4-methoxyphenyl)methyl]pyrazol-3-yl]benzimidazole-1-carboxylate (70 mg, 0.167 mmol) was added. The mixture was warmed to 23° C., stirred for 18 h, and concentrated. Brine (10 mL) was added, and the aq. phase was extracted with EtOAc (3×20 mL). The combined organic phases were washed with brine (20 mL), dried (MgSO₄), filtered, and concentrated. The product was purified by silica gel chromatography (4 g cartridge) eluting with hexanes and EtOAc (0-100%) to provide the title compound as a solid (52 mg; 56%). ¹H NMR (400 MHz, CDCl₃) δ 10.24 (s, 1H), 8.24 (dd, J=2.9, 0.5 Hz, 1H), 8.20 (dd, J=8.6, 0.5 Hz, 1H), 8.12-8.09 (m, 1H), 7.85-7.82 (m, 1H), 7.49-7.39 (m, 2H), 7.34-7.29 (m, 1H), 7.17 (s, 1H), 7.08-7.02 (m, 2H), 6.74-6.68 (m, 2H), 5.31 (s, 2H), 3.93 (s, 3H), 3.71 (s, 3H), 1.40 (s, 9H). m/z (ES⁺), [M+H]⁺ 555.5. HPLC (A05) t_{R}=2.78 min.

### Step 2: N-[5-(1H-benzimidazol-2-yl)-1H-pyrazol-3-yl]-5-methoxy-pyridine-2-carboxamide

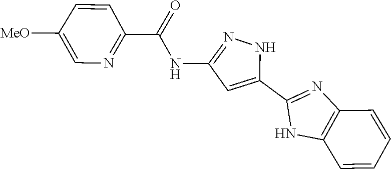 [SMILES: COc1ccc(C(=O)Nc2cc(-c3nc4ccccc4[nH]3)[nH]n2)nc1]

tert-Butyl 2-[2-[(4-methoxyphenyl)methyl]-5-[(5-methoxypyridine-2-carbonyl)amino]pyrazol-3-yl]benzimidazole-1-carboxylate (52 mg, 0.0938 mmol) was dissolved in DCM (1.50 mL). TFA (1.50 mL) was added, and the mixture was stirred at 23° C. for 4 h. The mixture was concentrated. The residue was diluted in sat. 1N NaOH (10 mL) and stirred for 5 min. The mixture was extracted with EtOAc (3×50 mL). The combined organic phases were washed with brine (20 mL), dried (MgSO₄), filtered, and concentrated. The residue was washed with water, MeCN, and hexanes to provide a 4:3 mixture of pyrazole tautomers of the title compound as a solid (14.92 mg, 48%). ¹H NMR (500 MHz, DMSO) δ 13.57 [12.94] (s, 1H), 13.07 [12.72] (s, 1H), 10.37 [11.36] (s, 1H), 8.42 [8.42] (d, J=2.7 Hz, 1H), 8.17 [8.17] (d, J=8.3 Hz, 1H), 7.64 [7.64] (d, J=6.7 Hz, 2H), 7.58-7.40 [7.58-7.40 ] (m, 1H), 7.43 [6.91] (s, 1H), 7.29-7.13 [7.29-7.13] (m, 2H), 3.95 [3.95] (s, 3H). m/z (ES+), [M+H]⁺ 334.7. HPLC (A05) t_{R}=2.06 min.

### EXAMPLE B18

### Preparation of Compounds A-13, A-14, A-16, A-17, A-23, A-32, A-35, and A-37

### Representative Scheme for the Synthesis of the Title Compounds

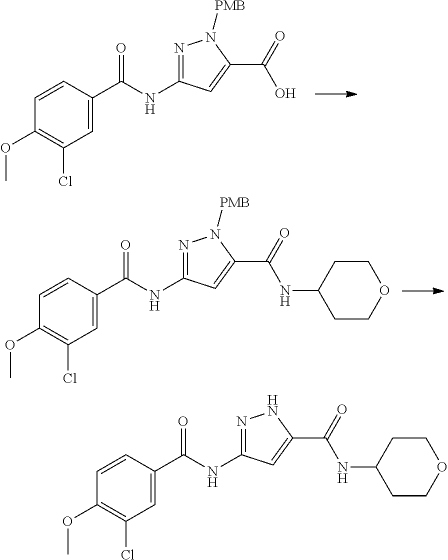

### Preparation of Compound A-13.

### Step 1: 5-[(3-chloro-4-methoxy-benzoyl)amino]-2-[(4-methoxyphenyl)-methyl]-N-tetrahydropyran-4-yl-pyrazole-3-carboxamide

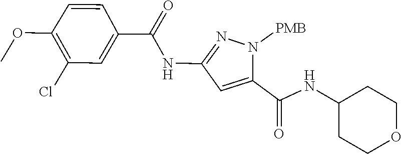

DIEA (52.3 μL, 0.300 mmol) was added to a solution of 5-[(3-chloro-4-methoxy-benzoyl)amino]-2-[(4-methoxyphenyl)methyl]pyrazole-3-carboxylic acid (M-2) (62.4 mg, 0.150 mmol) and tetrahydropyran-4-amine (20.2 μL, 0.195 mmol) in DMF (3 mL). HATU (68.4 mg, 0.180 mmol) was added, and the mixture was stirred at 23° C. for 2 h. Water (20 mL) was added. The mixture was filtered, and the solid was washed with water (3×15 mL) and dried to provide the title compound as a solid (67.7 mg, 91%). ¹H NMR (400 MHz, DMSO) δ 10.98 (s, 1H), 8.53 (d, J=7.8 Hz, 1H), 8.12 (d, J=2.2 Hz, 1H), 8.03 (dd, J=8.7, 2.2 Hz, 1H), 7.36 (s, 1H), 7.25 (d, J=8.8 Hz, 1H), 7.18 (d, J=8.7 Hz, 2H), 6.87 (d, J=8.7 Hz, 2H), 5.59 (s, 2H), 4.04-3.95 (m, 1H), 3.93 (s, 3H), 3.87 (d, J=9.7 Hz, 2H), 3.71 (s, 3H), 3.37 (dd, J=11.8, 9.9 Hz, 2H), 1.73 (dd, J=12.6, 2.6 Hz, 2H), 1.58 (qd, J=12.2, 4.5 Hz, 2H). m/z (ES⁺) [M+H]⁺: 499.9; HPLC (A05) t_{R}=2.41 min.

### Step 2: 3-[(3-chloro-4-methoxy-benzoyl)amino]-N-tetrahydropyran-4-yl-1H-pyrazole-5-carboxamide

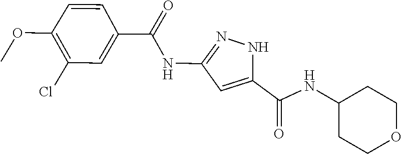 [SMILES: COc1ccc(C(=O)Nc2cc(C(=O)NC3CCOCC3)[nH]n2)cc1Cl]

5-[(3-Chloro-4-methoxy-benzoyl) amino]-2-[(4-methoxyphenyl) methyl]-N-tetrahydropyran-4-yl-pyrazole-3-carboxamide (64 mg, 0.128 mmol) was dissolved in DCM (1.50 mL). TFA (1.50 mL) was added. The mixture was stirred at 23° C. for 16 h and heated at 70° C. for 2 h. The mixture was concentrated. Sat. NaHCO₃ (10 mL) was added, and the aq. phase was extracted with EtOAc (3×25 mL). The combined organic phases were washed with brine (10 mL), dried (MgSO₄), filtered, and concentrated. The residue was purified by silica gel chromatography (4 g, cartridge) eluting with mixtures of DCM and MeOH (0-10%) to provide the title compound as a solid (31.2 mg, 64%). ¹H NMR (500 MHz, DMSO) δ 13.14 (s, 1H), 10.90 (s, 1H), 8.42 (d, J=7.7 Hz, 1H), 8.12 (d, J=1.9 Hz, 1H), 8.04 (dd, J=8.6, 1.9 Hz, 1H), 7.34 (d, J=1.7 Hz, 1H), 7.26 (d, J=8.7 Hz, 1H), 4.02-3.95 (m, 1H), 3.94 (s, 3H), 3.88 (d, J=9.7 Hz, 2H), 3.38 (t, J=11.1 Hz, 2H), 1.75 (d, J=12.7 Hz, 2H), 1.58 (ddd, J=16.2, 12.5, 4.5 Hz, 2H). m/z (ES⁺), [M+H]⁺: 379.7; HPLC (A05) t_{R}=2.02 min.

### Preparation of Compound A-14.

### Step 1: 5-[(3-chloro-4-methoxy-benzoyl) amino]-N-cyclohexyl-2-[(4-methoxyphenyl)methyl]pyrazole-3-carboxamide

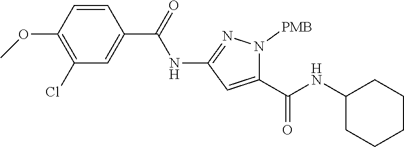

DIEA (41.9 μL, 0.240 mmol) was added to a solution of 5-[(3-chloro-4-methoxy-benzoyl)amino]-2-[(4-methoxyphenyl) methyl] pyrazole-3-carboxylic acid (M-2) (50 mg, 0.120 mmol) and cyclohexanamine (17.9 μL, 0.156 mmol) in DMF (2 mL). HATU (54.9 mg, 0.144 mmol) was added, and the mixture was stirred at 23° C. for 2 h. Water (20 mL) was added. The mixture was filtered, and the solid was washed with water (3×15 mL) and dissolved in EtOAc (30 mL). The organic phase was dried (MgSO₄), filtered, and concentrated to provide the title compound as a solid (56.2 mg, 94%). ¹H NMR (400 MHz, DMSO) δ 10.96 (s, 1H), 8.43 (d, J=8.0 Hz, 1H), 8.12 (d, J=2.2 Hz, 1H), 8.03 (dd, J=8.7, 2.2 Hz, 1H), 7.34 (s, 1H), 7.24 (d, J=8.8 Hz, 1H), 7.18 (d, J=8.7 Hz, 2H), 6.87 (d, J=8.8 Hz, 2H), 5.59 (s, 2H), 3.93 (s, 3H), 3.77-3.72 (m, 1H), 3.71 (s, 3H), 1.85-1.68 (m, 4H), 1.60 (d, J=12.5 Hz, 1H), 1.37-1.25 (m, 4H), 1.14-1.01 (m, 1H). m/z (ES⁺) [M+H]⁺: 496.9; HPLC (A05) t_{R}=2.71 min.

### Step 2: 3-[(3-chloro-4-methoxy-benzoyl) amino]-N-cyclohexyl-1H-pyrazole-5-carboxamide

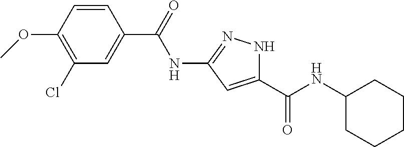 [SMILES: COc1ccc(C(=O)Nc2cc(C(=O)NC3CCCCC3)[nH]n2)cc1Cl]

A solution of 5-[(3-chloro-4-methoxy-benzoyl) amino]-N-cyclohexyl-2-[(4-methoxyphenyl) methyl]pyrazole-3-carboxamide (56 mg, 0.113 mmol) in TFA (2 mL) was heated to 70° C. for 30 min. The mixture was concentrated. Sat. NaHCO₃ (10 mL) was added, and the aq. phase was extracted with EtOAc (3×25 mL). The combined organic phases were washed with brine (10 mL), dried (MgSO₄), filtered, and concentrated. The residue was purified by silica gel chromatography (4 g, cartridge) eluting with mixtures of DCM and MeOH (0-10%) to provide the title compound as a solid (26.1 mg, 62%). ¹H NMR (500 MHz, DMSO) δ 13.09 (s, 1H), 10.88 (s, 1H), 8.31 (d, J=8.0 Hz, 1H), 8.12 (d, J=2.0 Hz, 1H), 8.04 (dd, J=8.7, 2.0 Hz, 1H), 7.33 (d, J=1.8 Hz, 1H), 7.26 (d, J=8.7 Hz, 1H), 3.94 (s, 3H), 3.78-3.68 (m, 1H), 1.84-1.69 (m, 4H), 1.61 (d, J=12.5 Hz, 1H), 1.37-1.21 (m, 4H), 1.19-1.06 (m, 1H). m/z (ES⁺), [M+H]⁺: 378.4; HPLC (A05) t_{R}=2.34 min.

### Preparation of Compound A-16.

### Step 1: 5-[(3-chloro-4-methoxy-benzoyl)amino]-2-[(4-methoxyphenyl)methyl]-N-(oxetan-3-yl) pyrazole-3-carboxamide

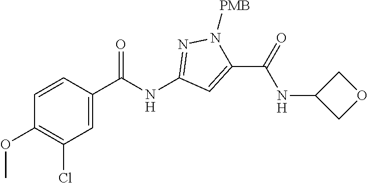

DIEA (50.3 μL, 0.289 mmol) was added to a solution of 5-[(3-chloro-4-methoxy-benzoyl)amino]-2-[(4-methoxyphenyl) methyl] pyrazole-3-carboxylic acid (M-2) (60 mg, 0.144 mmol) and oxetan-3-amine (14.9 μL, 0.188 mmol) in DMF (2 mL). HATU (65.8 mg, 0.173 mmol) was added, and the mixture was stirred at 23° C. for 2 h. Water (20 mL) was added. The mixture was filtered, and the solid was washed with water (3×15 mL) and dissolved in EtOAc (30 mL). The organic phase was dried (MgSO₄), filtered, and concentrated to provide the title compound as a solid (56 mg, 82%). m/z (ES⁺) [M+H]⁺:471.7; HPLC (A05) t_{R}=2.33 min.

### Step 2: 3-[(3-chloro-4-methoxy-benzoyl) amino]-N-(oxetan-3-yl)-1H-pyrazole-5-carboxamide

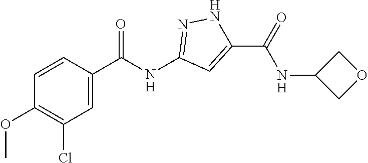 [SMILES: COc1ccc(C(=O)Nc2cc(C(=O)NC3COC3)[nH]n2)cc1Cl]

A solution of 5-[(3-chloro-4-methoxy-benzoyl) amino]-2-[(4-methoxyphenyl) methyl]-N-(oxetan-3-yl) pyrazole-3-carboxamide (56 mg, 0.119 mmol) in TFA (2 mL) was heated to 70° C. for 30 min. The mixture was concentrated. Sat. NaHCO₃ (10 mL) was added, and the aq. phase was extracted with EtOAc (3×25 mL). The combined organic phases were washed with brine (10 mL), dried (MgSO₄), filtered and concentrated. The residue was purified by silica gel chromatography (4 g, cartridge) eluting with mixtures of DCM and MeOH (0-10%) to provide the title compound as a solid (27.9 mg, 67%). ¹H NMR (500 MHz, DMSO) δ 13.41 (s, 1H), 10.94 (s, 1H), 8.13 (s, 1H), 8.03 (d, J=8.3 Hz, 1H), 7.26 (d, J=8.6 Hz, 1H), 7.00 (s, 1H), 4.85 (t, J=5.2 Hz, 1H), 4.44 (t, J=7.9 Hz, 1H), 4.36-4.19 (m, 2H), 3.94 (s, 3H), 3.64-3.54 (m, 1H), 3.54-3.41 (m, 1H). m/z (ES⁺) [M+H]⁺:351.2; HPLC (A05) t_{R}=2.14 min.

### Preparation of Compound A-17.

### Step 1: 5-[(3-chloro-4-methoxy-benzoyl)amino]-2-[(4-methoxyphenyl)-methyl]-N-tetrahydrofuran-3-yl-pyrazole-3-carboxamide

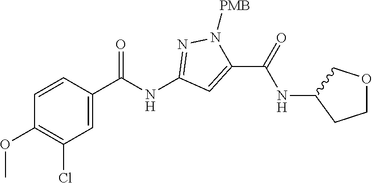

5-[(3-Chloro-4-methoxy-benzoyl)amino]-2-[(4-methoxyphenyl)methyl]pyrazole-3-carboxylic acid (M-2) (50 mg, 0.120 mmol) and HATU (45.7 mg, 0.120 mmol) were dissolved in DMF (1.25 mL) at 0° C. DIEA (041.2 μL, 0.240 mmol) was added, and the mixture was stirred at 0° C. for 5 min. Tetrahydrofuran-3-amine (10.5 mg, 0.120 mmol) was added. The mixture was warmed to 23° C. and stirred for 3 h. The mixture was concentrated. Water (10 mL) was added, and the mixture was filtered. The solid was dried under high vacuum to provide the title compound as a solid (55 mg, 94%). ¹H NMR (400 MHz, DMSO) δ 10.98 (s, 1H), 8.76 (d, J=6.6 Hz, 1H), 8.12 (d, J=2.2 Hz, 1H), 8.03 (dd, J=8.7, 2.2 Hz, 1H), 7.41 (s, 1H), 7.25 (d, J=8.8 Hz, 1H), 7.20-7.16 (m, 2H), 6.89-6.85 (m, 2H), 5.59 (s, 2H), 4.45-4.40 (m, 1H), 3.93 (s, 3H), 3.87-3.82 (m, 2H), 3.73-3.70 (m, 4H), 3.57 (dd, J=9.0, 4.3 Hz, 1H), 2.16-2.11 (m, 1H), 1.95-1.90 (m, 1H). m/z (ES⁺), [M+H]⁺ 487.2. HPLC (A05) t_{R}=2.37 min.

### Step 2: 3-[(3-chloro-4-methoxy-benzoyl)amino]-N-tetrahydrofuran-3-yl-1H-pyrazole-5-carboxamide

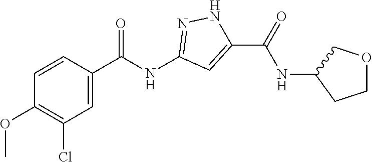 [SMILES: COc1ccc(C(=O)Nc2cc(C(=O)NC3CCOC3)[nH]n2)cc1Cl]

A solution of 5-[(3-chloro-4-methoxy-benzoyl)amino]-2-[(4-methoxyphenyl)methyl]-N-tetrahydrofuran-3-yl-pyrazole-3-carboxamide (55 mg, 0.113 mmol) in TFA (3 mL) was heated to 70° C. for 1 h. The mixture was concentrated. Sat. NaHCO₃ (10 mL) was added, and the aq. phase was extracted with EtOAc (3×20 mL). The combined organic phases were washed with brine (20 mL), dried (MgSO₄), filtered, and concentrated. The residue was purified by silica gel chromatography (12 g cartridge) eluting with DCM and MeOH (0-10%) to provide the title compound as a solid (20 mg, 48%). ¹H NMR (500 MHz, DMSO) δ 13.14 (s, 1H), 10.90 (s, 1H), 8.65 (d, J=7.0 Hz, 1H), 8.12 (d, J=2.2 Hz, 1H), 8.04 (dd, J=8.7, 2.2 Hz, 1H), 7.38 (d, J=2.1 Hz, 1H), 7.26 (d, J=9.0 Hz, 1H), 4.46-4.41 (m, 1H), 3.94 (s, 3H), 3.87-3.82 (m, 2H), 3.72 (td, J=8.2, 5.9 Hz, 1H), 3.59 (dd, J=9.1, 4.3 Hz, 1H), 2.18-2.12 (m, 1H), 1.95-1.89 (m, 1H). m/z (ES⁺), [M+H]⁺: 365.3; HPLC (A05) t_{R}=1.99 min.

### Preparation of Compound A-23.

### Step 1: N-benzyl-5-[(3-chloro-4-methoxy-benzoyl)amino]-2-[(4-methoxyphenyl)-methyl]pyrazole-3-carboxamide

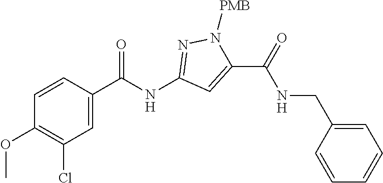

5-[(3-Chloro-4-methoxy-benzoyl)amino]-2-[(4-methoxyphenyl)methyl]pyrazole-3-carboxylic acid (M-2) (50 mg, 0.120 mmol) and HATU (45.7 mg, 0.120 mmol) were dissolved in DMF (1.25 mL) at 0° C. DIEA (41.2 μL, 0.240 mmol) was added, and the mixture was stirred at 0° C. for 5 min. Benzylamine (13.1 μL, 0.120 mmol) was added. The mixture was warmed to 23° C. and stirred for 18 h. The mixture was concentrated. Water (10 mL) was added, and the aq. phase was extracted with EtOAc (3×20 mL). The combined organic phases were washed with brine (20 mL), dried (MgSO₄), filtered, and concentrated. The product was purified by silica gel chromatography (12 g cartridge) eluting with DCM and MeOH (0-5%) to provide the title compound as a solid (43.0 g, 71%). ¹H NMR (400 MHz, DMSO) δ 11.00 (s, 1H), 9.21 (t, J=6.1 Hz, 1H), 8.12 (d, J=2.2 Hz, 1H), 8.03 (dd, J=8.7, 2.2 Hz, 1H), 7.38 (s, 1H), 7.36-7.22 (m, 6H), 7.18-7.13 (m, 2H), 6.88-6.83 (m, 2H), 5.61 (s, 2H), 4.43 (d, J=6.0 Hz, 2H), 3.93 (s, 3H), 3.72 (s, 3H). m/z (ES⁺), [M+H]⁺ 505.5. HPLC (A05) t_{R}=2.56 min.

### Step 2: N-benzyl-3-[(3-chloro-4-methoxy-benzoyl)amino]-1H-pyrazole-5-carboxamide.

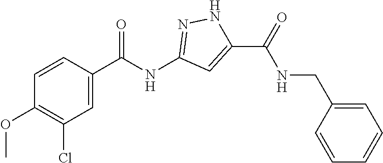 [SMILES: COc1ccc(C(=O)Nc2cc(C(=O)NCc3ccccc3)[nH]n2)cc1Cl]

A solution of N-benzyl-5-[(3-chloro-4-methoxy-benzoyl)amino]-2-[(4-methoxyphenyl)methyl]-pyrazole-3-carboxamide (43 mg, 0.0852 mmol) in TFA (2 mL) was heated to 70° C. for 2 h. The mixture was concentrated. Sat. NaHCO₃ (10 mL) was added, and the aq. phase was extracted with EtOAc (3×20 mL). The combined organic phases were washed with brine (20 mL), dried (MgSO₄), filtered, and concentrated. The residue was purified by HPLC prep (BEH C18 30×150) eluting with water (10 mM (NH₄)(HCO₂)) and MeCN (40-60%) to provide the title compound as a solid (8 mg, 24%). ¹H NMR (400 MHz, DMSO) δ 13.20 (s, 1H), 10.91 (s, 1H), 9.11 (t, J=5.8 Hz, 1H), 8.12 (s, 1H), 8.04 (d, J=8.3 Hz, 1H), 7.35-7.31 (m, 5H), 7.26 (d, J=8.7 Hz, 2H), 4.45 (d, J=5.8 Hz, 2H), 3.94 (s, 3H). m/z (ES⁺), [M+H]⁺ 385.3. HPLC (A05) t_{R}=2.28 min.

### Preparation of Compound A-32.

### Step 1: N-[5-(benzylcarbamoyl)-1-[(4-methoxyphenyl)methyl]pyrazol-3-yl]-6-(4-methylpiperazin-1-yl)pyridine-3-carboxamide

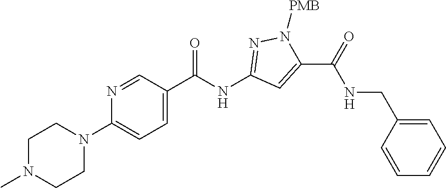

DIEA (54.1 μL, 0.311 mmol) was added to a solution of HATU (37.1 mg, 0.0977 mmol) and 2-[(4-methoxyphenyl)methyl]-5-[[6-(4-methylpiperazin-1-yl)pyridine-3-carbonyl]amino]pyrazole-3-carboxylic acid (M-5) (40 mg, 0.0888 mmol) in DMF (1 mL) at 0° C. The mixture was stirred at 0° C. for 20 min. Benzylamine (14.5 μL, 0.133 mmol) was added. The mixture was warmed to 23° C., stirred for 18 h, and poured into water (10 mL). The mixture was filtered, and the solid was purified by silica gel chromatography (12 g cartridge) eluting with mixtures of DCM and MeOH (0-10%) to provide the title compound as a solid (37 mg, 77%). ¹H NMR (500 MHz, DMSO) δ 10.80 (s, 1H), 9.20 (t, J=6.1 Hz, 1H), 8.76 (d, J=2.6 Hz, 1H), 8.11 (dd, J=9.1, 2.5 Hz, 1H), 7.38 (s, 1H), 7.36-7.31 (m, 2H), 7.30-7.24 (m, 3H), 7.18-7.13 (m, 2H), 6.92-6.81 (m, 3H), 5.61 (s, 2H), 4.44 (d, J=6.1 Hz, 2H), 3.72 (s, 3H), 3.66-3.56 (m, 4H), 2.43-2.29 (m, 4H), 2.22 (s, 3H). m/z (ES⁺), [M+H]⁺ 540.6. HPLC (A05) t_{R}=2.06 min.

### Step 2: N-[5-(benzylcarbamoyl)-1H-pyrazol-3-yl]-6-(4-methylpiperazin-1-yl)pyridine-3-carboxamide

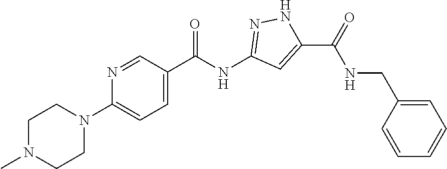 [SMILES: CN1CCN(c2ccc(C(=O)Nc3cc(C(=O)NCc4ccccc4)[nH]n3)cn2)CC1]

TFA (1 mL) was added to N-[5-(benzylcarbamoyl)-1-[(4-methoxyphenyl)methyl]pyrazol-3-yl]-6-(4-methylpiperazin-1-yl)pyridine-3-carboxamide (35.7 mg, 0.0662 mmol), and the mixture was heated at 70° C. for 30 min. MeOH (5 mL) and 2N NaOH (6.6 mL) were added. Water (5 mL) was added, and the pH was adjusted to pH 7 with 1N HCl. The mixture was filtered, and the solid was washed with water. The solid was purified by HPLC (BEH C18 30×150) eluting with water (10 mM (NH₄)(HCO₃)) and MeCN (30-50%) to provide the title compound as a solid (11.2 mg, 40%). ¹H NMR (400 MHz, DMSO) δ 13.17 (br, 1H), 10.71 (br, 1H), 9.10 (br, 1H), 8.76 (s, 1H), 8.21-7.98 (m, 1H), 7.38-7.30 (m, 4H), 7.29-7.21 (m, 1H), 6.93-6.85 (m, J=8.5 Hz, 1H), 4.45 (d, J=6.0 Hz, 2H), 3.67-3.57 (m, 4H), 2.44-2.35 (m, 4H), 2.23 (s, 3H). m/z (ES⁺), [M+H]⁺ 420.6. HPLC (A05) t_{R}=1.72 min.

### Preparation of Compound A-35.

### Step 1: N-[1-[(4-methoxyphenyl)methyl]-5-[[(1R)-1-phenylethyl]-carbamoyl]-pyrazol-3-yl]-6-(4-methylpiperazin-1-yl)pyridine-3-carboxamide

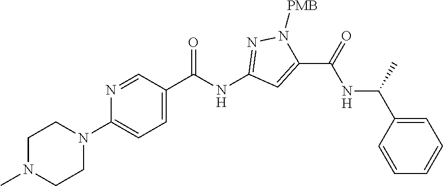

2-[(4-Methoxyphenyl)methyl]-5-[[6-(4-methylpiperazin-1-yl)pyridine-3-carbonyl]amino]-pyrazole-3-carboxylic acid (M-5) (40 mg, 0.0888 mmol) and HATU (33.8 mg, 0.0888 mmol) were dissolved in DMF (1 mL) at 0° C. DIEA (30.4 μL, 0.178 mmol) was added, and the mixture was stirred at 0° C. for 5 min. (1R)-1-phenylethanamine (11.4 μL, 0.0888 mmol) was added. The mixture was warmed to 23° C., stirred for 18 h, and concentrated. Water (10 mL) was added, and the aq. phase was extracted with EtOAc (3×20 mL). The combined organic phases were washed with brine (20 mL), dried (MgSO₄), filtered, and concentrated. The residue was purified by silica gel chromatography (12 g cartridge) eluting with DCM and MeOH (0-10%) to provide the title compound as a solid (37 mg, 75%). ¹H NMR (400 MHz, DMSO) δ 10.78 (s, 1H), 9.02 (d, J=8.4 Hz, 1H), 8.76 (d, J=2.6 Hz, 1H), 8.11 (dd, J=9.1, 2.6 Hz, 1H), 7.44 (s, 1H), 7.37-7.33 (m, 3H), 7.27-7.22 (m, 2H), 7.11 (d, J=8.8 Hz, 2H), 6.86 (d, J=8.8 Hz, 1H), 6.84-6.80 (m, 2H), 5.54 (s, 2H), 5.18-5.09 (m, 1H), 3.70 (s, 3H), 3.64-3.58 (m, 4H), 2.41-2.35 (m, 4H), 2.21 (s, 3H), 1.46 (d, J=7.1 Hz, 3H). m/z (ES⁺), [M+H]⁺ 554.7. HPLC (A05) t_{R}=2.11 min.

### Step 2: 6-(4-methylpiperazin-1-yl)-N-[5-[[(1R)-1-phenylethyl]-carbamoyl]-1H-pyrazol-3-yl]pyridine-3-carboxamide

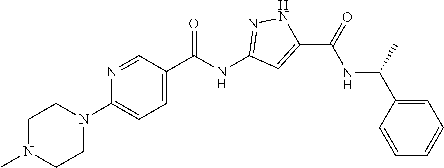 [SMILES: C[C@@H](NC(=O)c1cc(NC(=O)c2ccc(N3CCN(C)CC3)nc2)n[nH]1)c1ccccc1]

A solution of N-[1-[(4-methoxyphenyl)methyl]-5-[[(1R)-1-phenylethyl]carbamoyl]pyrazol-3-yl]-6-(4-methylpiperazin-1-yl)pyridine-3-carboxamide (37 mg, 0.0668 mmol) in TFA (2 mL) was heated at 70° C. for 1 h. The mixture was concentrated. Sat. NaHCO₃ (10 mL) was added, and the aq. phase was extracted with EtOAc (3×20 mL). The combined organic phases were washed with brine (20 mL), dried (MgSO₄), filtered, and concentrated to provide the title compound as a solid (22 mg, 76%). ¹H NMR (400 MHz, DMSO) δ 13.06 (s, 1H), 10.69 (s, 1H), 8.89 (d, J=7.4 Hz, 1H), 8.76 (d, J=1.5 Hz, 1H), 8.11 (d, J=9.2 Hz, 1H), 7.45-7.36 (m, 3H), 7.33 (t, J=7.6 Hz, 2H), 7.23 (t, J=7.2 Hz, 1H), 6.88 (d, J=8.9 Hz, 1H), 5.20-5.09 (m, 1H), 3.64-3.59 (m, 4H), 2.41-2.36 (m, 4H), 2.22 (s, 3H), 1.47 (d, J=7.1 Hz, 3H). m/z (ES⁺), [M+H]⁺ 434.5. HPLC (A05) t_{R}=1.84 min.

### Preparation of Compound A-37 (4-[[[3-[[6-(4-methylpiperazin-1-yl)pyridine-3-carbonyl]amino]-1H-pyrazole-5-carbonyl]amino]methyl]benzoic acid)

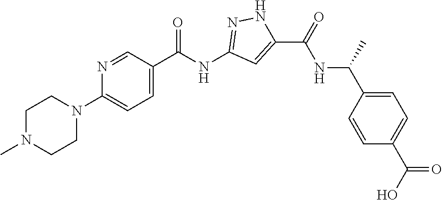 [SMILES: C[C@@H](NC(=O)c1cc(NC(=O)c2ccc(N3CCN(C)CC3)nc2)n[nH]1)c1ccc(C(=O)O)cc1]

2-[(4-Methoxyphenyl)methyl]-5-[[6-(4-methylpiperazin-1-yl)pyridine-3-carbonyl]amino]-pyrazole-3-carboxylic acid (M-5) (60 mg, 0.133 mmol) and HATU (50.6 mg, 0.133 mmol) were dissolved in DMF (1.50 mL) at 0° C. DIEA (45.6 μL, 0.266 mmol) was added, and the mixture was stirred at 23° C. for 30 min. 4-(Aminomethyl)benzoic acid (20.1 mg, 0.133 mmol) was added, and the mixture was stirred for 18 h. The mixture was concentrated. The residue was purified by silica gel chromatography (25 g cartridge) eluting with DCM and MeOH (0-40%). The purified residue (m/z (ES+), [M+H]+ 584.7. HPLC (A05) t_{R}=1.89 min) was dissolved in TFA (4 mL). The mixture was heated at 70° C. for 1 h and concentrated. Sat. NaHCO₃ (10 mL) was added. The mixture was filtered, and the solid was purified by reverse phase chromatography (BEH C18 30×150) eluting with water (10 mM (NH₄)(HCO₂)) and MeCN (14-24%) to provide the title compound as a solid (4 mg, 7%). ¹H NMR (400 MHz, DMSO) δ 13.18 (s, 1H), 12.88 (s, 1H), 10.77-10.67 (m, 1H), 9.21-9.09 (m, 1H), 8.75 (s, 1H), 8.11 (d, J=9.1 Hz, 1H), 7.91 (d, J=8.2 Hz, 2H), 7.42 (d, J=8.0 Hz, 2H), 7.38-7.28 (m, 1H), 6.89 (s, 1H), 4.51 (s, 2H), 3.62 (s, 4H), 2.40-2.37 (m, 4H), 2.22 (s, 3H). m/z (ES⁺), [M+H]⁺ 464.5. HPLC (A05) t_{R}=1.64 min.

### EXAMPLE B19

### Preparation of Compounds A-30 and A-122

### Representative Scheme for the Synthesis of Compounds A-30 and A-122.

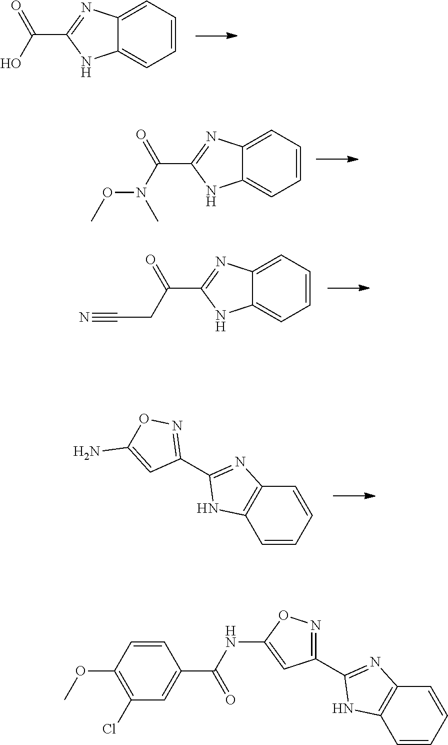

### Preparation of Compound A-30.

### Step 1: N-methoxy-N-methyl-1H-benzimidazole-2-carboxamide

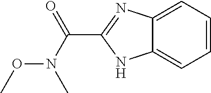

1H-Benzimidazole-2-carboxylic acid (3.00 g, 18.5 mmol) and HATU (8.44 g, 22.2 mmol) were dissolved in DMF (80 mL) at 0° C. DIEA (9.50 mL, 55.5 mmol) was added, and the mixture was stirred at 0° C. for 5 min. N-Methoxymethanamine hydrochloride (2.17 g, 22.2 mmol) was added. The mixture was warmed to 23° C. and stirred for 48 h. The mixture was concentrated. Water (100 mL) was added, and the aq. phase was extracted with EtOAc (3×150 mL). The combined organic phases were washed with brine (100 mL), dried (MgSO₄), filtered, and concentrated. The product was purified by silica gel chromatography (220 g cartridge) eluting with DCM and MeOH (0-5%) to provide the title compound as a solid (2.30 g, 87%). ¹H NMR (500 MHz, DMSO) δ 13.19 (s, 1H), 7.80-7.69 (m, 1H), 7.59-7.51 (m, 1H), 7.34-7.25 (m, 2H), 3.83 (s, 3H), 3.41 (s, 3H). m/z (ES⁺), [M+H]⁺ 207.1. HPLC (A05) t_{R}=1.78 min.

### Step 2: 3-(1H-benzimidazol-2-yl)-3-oxo-propanenitrile

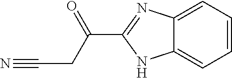 [SMILES: N#CCC(=O)c1nc2ccccc2[nH]1]

MeLi—LiBr (1.50 M, 48.7 mL, 73.1 mmol) in Et₂O was added dropwise to a solution of acetonitrile (4.20 mL, 80.4 mmol) in THF (40 mL) at −78° C., and the solution was stirred for 30 min. A solution of N-methoxy-N-methyl-1H-benzimidazole-2-carboxamide (3.00 g, 14.6 mmol) in THF (40 mL) was added dropwise at −78° C. The mixture was warmed to 23° C. and stirred for 2 h. Sat. NH₄Cl (300 mL) was added, and the aq. phase was extracted EtOAc (3×200 mL). The combined organic phases were washed with brine (200 mL), dried (MgSO₄), filtered, and concentrated to provide the title compound as a solid (1.93 g, 71%). m/z (ES+), [M+H]⁺ 186.1. HPLC (A05) t_{R}=0.31 min.

### Step 3: 3-(1H-benzimidazol-2-yl)isoxazol-5-amine

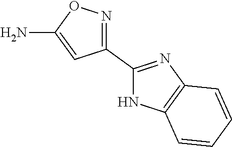 [SMILES: Nc1cc(-c2nc3ccccc3[nH]2)no1]

3-(1H-Benzimidazol-2-yl)-3-oxo-propanenitrile (1.00 g, 5.40 mmol) was suspended in water (7 mL) at 23° C. with NaOH (0.270 g, 6.75 mmol) and hydroxylamine sulfate (0.975 g, 5.94 mmol). The pH of the solution was adjusted to 8 by adding NaOH. The solution was heated to 45° C. for 72 h. HCl (12.0 M, 0.0792 mL, 0.950 mmol) was added, and the solution was heated at 50° C. for 2.5 h. After cooling to 23° C., the pH of the solution was increased to pH 11 by adding concentrated aq. NaOH. The aq. phase was extracted with EtOAc (3×100 mL), and the combined organic phases were washed with brine (150 mL), dried (MgSO₄), filtered, and concentrated to provide the title compound as a solid (446 mg, 41%). ¹H NMR (400 MHz, DMSO) δ 13.01 (s, 1H), 7.68 (d, J=7.6 Hz, 1H), 7.49 (d, J=8.3 Hz, 1H), 7.23 (dt, J=14.9, 5.9 Hz, 2H), 6.99 (s, 2H), 5.51 (s, 1H).

### Step 4: N-[3-(1H-benzimidazol-2-yl)isoxazol-5-yl]-3-chloro-4-methoxy-benzamide

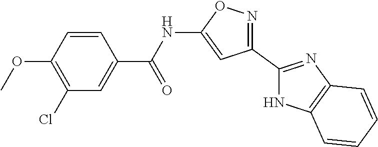 [SMILES: COc1ccc(C(=O)Nc2cc(-c3nc4ccccc4[nH]3)no2)cc1Cl]

3-(1H-Benzimidazol-2-yl)isoxazol-5-amine (38.6 mg, 0.193 mmol), 3-chloro-4-methoxy-benzoic acid (30 mg, 0.161 mmol), and NEt₃ (0.0672 mL, 0.482 mmol) were dissolved in DMF (1 mL) at 0° C., and the solution was stirred for 5 min. T3P (50.0% in EtOAc, 0.115 mL, 0.193 mmol) was added, and the mixture was stirred at 23° C. for 3 h and heated at 70° C. for 30 min. Water (10 mL) was added, and the aq. phase was extracted with EtOAc (3×20 mL). The combined organic phases were washed with brine (20 mL), dried (MgSO₄), filtered, and concentrated. The product was purified by silica gel chromatography (25 g cartridge) eluting with hexanes and EtOAc (0-100%) followed by prep-HPLC (BEH C18 30×150) eluting with water (10 mM (NH₄)(HCO₂)) and MeCN (41-61%) to provide the title compound as a solid (10 mg, 17%). ¹H NMR (400 MHz, DMSO) δ 8.28 (s br, 1H), 8.16 (d, J=2.1 Hz, 1H), 8.06 (dd, J=8.6, 2.1 Hz, 1H), 7.79-7.47 (m, 2H), 7.29-7.19 (m, 3H), 6.86 (s, 1H), 3.93 (s, 3H). m/z (ES⁺), [M+H]⁺ 369.3. HPLC (A05) t_{R}=2.33 min.

### Preparation of Compound A-122.

### Step 1: 3-(1H-benzimidazol-2-yl)-2-methyl-3-oxo-propanenitrile

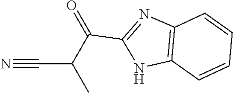

1.50 M MeLi—LiBr in Et₂O(1.50 M, 10.7 mL, 16.0 mmol) was added dropwise to a solution of propanenitrile (1.27 mL, 18.0 mmol) in THF (12 mL) at −78° C., and the solution was stirred for 30 min. A solution of N-methoxy-N-methyl-1H-benzimidazole-2-carboxamide (A-2, Step 1) (821 mg, 4.00 mmol) in THF (7 mL) was added dropwise at −78° C. The mixture was stirred for 2 h. After warming to 23° C., water (100 mL) was added, and the aq. phase was extracted with EtOAc (3×75 mL). The aq. phase was acidified with 1M HCl until a precipitate formed. The aq. phase was extracted with EtOAc (100 mL). The organic phase was washed with brine (50 mL), dried (Na₂SO₄), filtered, and concentrated to provide the title compound as a solid (144 mg, 18%). ¹H NMR (400 MHz, DMSO) δ 13.65 (s, 1H), 7.87 (d, J=8.2 Hz, 1H), 7.60 (d, J=8.2 Hz, 1H), 7.44 (t, J=7.1 Hz, 1H), 7.36 (t, J=7.6 Hz, 1H), 5.13 (q, J=7.3 Hz, 1H), 1.64 (d, J=7.3 Hz, 3H). m/z (ES⁺), [M+H]⁺ 200.0. HPLC (A05) t_{R}=1.97 min.

### Step 2: 5-(1H-benzimidazol-2-yl)-4-methyl-1H-pyrazol-3-amine

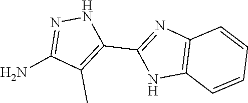 [SMILES: Cc1c(N)n[nH]c1-c1nc2ccccc2[nH]1]

NH₂NH₂—H₂O (0.0351 mL, 0.723 mmol) was added to a solution of 3-(1H-benzimidazol-2-yl)-2-methyl-3-oxo-propanenitrile (144 mg, 0.723 mmol) in EtOH (8 mL), and the mixture was refluxed for 18 h. The mixture was concentrated to provide the title compound as a solid (150 mg, 97%). ¹H NMR (400 MHz, DMSO) δ 12.33 (s, 1H), 11.77 (s, 1H), 7.60 (s, 1H), 7.44 (s, 1H), 7.13 (d, J=5.2 Hz, 2H), 4.83 (s, 2H), 2.21 (s, 3H). m/z (ES⁺), [M+H]⁺ 214.1. HPLC (A05) t_{R}=1.62 min.

### Step 3: N-[5-(1H-benzimidazol-2-yl)-4-methyl-1H-pyrazol-3-yl]-3-chloro-4-methoxy-benzamide

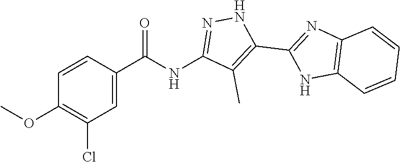 [SMILES: COc1ccc(C(=O)Nc2n[nH]c(-c3nc4ccccc4[nH]3)c2C)cc1Cl]

3-Chloro-4-methoxy-benzoic acid (65.6 mg, 0.352 mmol) and HATU (0.134 g, 0.352 mmol) were dissolved in DMF (2 mL) at 0° C. DIEA (0.120 mL, 0.703 mmol) was added, and the mixture was stirred at 0° C. for 5 min. 5-(1H-Benzimidazol-2-yl)-4-methyl-1H-pyrazol-3-amine (75 mg, 0.352 mmol) was added, and the mixture was stirred at 23° C. for 18 h. The mixture was concentrated, and water (40 mL) was added. The aq. phase was extracted with EtOAc (3×40 mL). The combined organic phases were washed with brine (40 mL), dried (Na₂SO₄), filtered, and concentrated. The residue was dissolved in DMF (3 mL) and heated at 100° C. for 18 h. The mixture was concentrated. The residue was purified by preparative HPLC (BEH C18 30×150) eluting with water (10 mM (NH₄)(HCO₃)) and MeCN (45-55%) to provide the title compound as a solid in a 3:1 mixture of tautomers (20.8 mg, 16%). ¹H NMR (400 MHz, DMSO) δ 13.24 (s, 1H), 12.63 [12.45] (s, 1H), 10.33 [10.24] (s, 1H), 8.13 (s, 1H), 8.03 (dd, J=8.7, 2.0 Hz, 1H), [7.69] 7.64 (t, J=8.4 Hz, 1H), [7.58] 7.46 (d, J=7.2 Hz, 1H), 7.34 [7.29] (t, J=11.0 Hz, 1H), [7.24] 7.16 (m, 2H), 3.96 (s, 3H), 2.34 [2.20] (s, 3H). m/z (ES⁺), [M+H]⁺ 383.1. HPLC (A05) t_{R}=2.18 min.

### EXAMPLE B20

### Preparation of Compounds A-34, A-58, A-79, A-106, and A-127

### Representative Scheme for Synthesis of the Title Compounds.

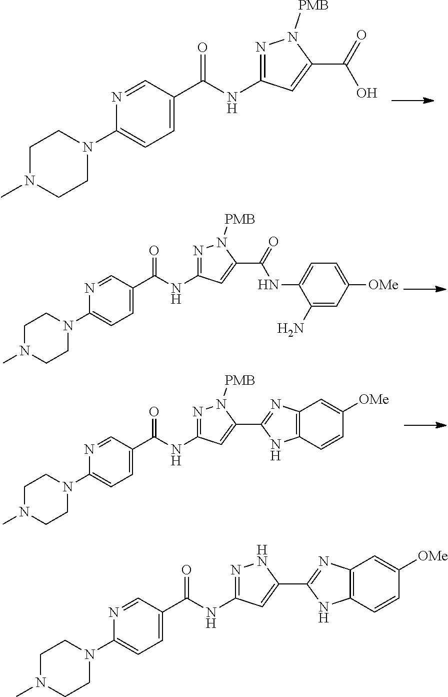

### Preparation of Compound A-34.

### Steps 1 and 2: N-[5-(6-methoxy-1H-benzimidazol-2-yl)-1-[(4-methoxyphenyl)-methyl]pyrazol-3-yl]-6-(4-methylpiperazin-1-yl)pyridine-3-carboxamide

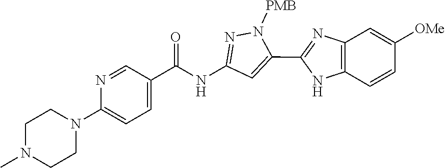

2-[(4-Methoxyphenyl)methyl]-5-[ [6-(4-methylpiperazin-1-yl)pyridine-3-carbonyl]amino]-pyrazole-3-carboxylic acid (M-5) (50 mg, 0.111 mmol) and HATU (0.0422 g, 0.111 mmol) were dissolved in DMF (1.50 mL) at 0° C. DIEA (76.0 μL, 0.444 mmol) was added, and the mixture was stirred at 0° C. for 5 min. 4-Methoxybenzene-1,2-diamine dihydrochloride (23.4 mg, 0.111 mmol) was added. The mixture was warmed to 23° C., stirred for 18 h, and concentrated. Water (10 mL) was added, and the aq. phase was extracted with EtOAc (3×20 mL). The combined organic phases were washed with brine (20 mL), dried (MgSO₄), filtered, and concentrated. The residue (m/z (ES⁺), [M+H]⁺ 571.7. HPLC (A05) t_{R}=1.96 min) was dissolved in AcOH (3 mL). The mixture was stirred at 60° C. for 18 h and concentrated. Sat. NaHCO₃ (30 mL) was added, and the aq. phase was extracted with EtOAc (3×20 mL). The combined organic phases were washed with brine (20 mL), dried (MgSO₄), filtered, and concentrated. The product was purified by silica gel chromatography (12 g cartridge) eluting with DCM and MeOH (0-10%) to provide the title compound as a solid in a 3:2 mixture of pyrazole tautomers (33 mg, 54%). ¹H NMR (400 MHz, DMSO) δ [12.94] 12.91 (s, 1H), 10.81 [10.81] (s, 1H), 8.74 [8.74] (d, J=2.5 Hz, 1H), 8.09 [8.09] (d, J=6.8 Hz, 1H), 7.55 [7.55] (d, J=8.8 Hz, 1H), 7.44 [7.44] (d, J=2.9 Hz, 1H), 7.37 [7.37] (d, J=8.5 Hz, 1H), 7.22-7.15 [7.22-7.15] (m, 3H), 6.94 [6.94] (d, J=2.5 Hz, 3H), 6.87-6.77 [6.87-6.77] (m, 4H), [5.97] 5.94 (s, 2H), 3.78 [3.76] (s, 3H), 3.65 [3.64] (s, 3H), 3.58 [3.58] (s, 4H), 2.35 [2.35] (s, 4H), 2.18 [2.18] (s, 3H). m/z (ES⁺), [M+H] 554.0. HPLC (A05) t_{R}=2.02 min.

### Step 3: N-[5-(5-methoxy-1H-benzimidazol-2-yl)-1H-pyrazol-3-yl]-6-(4-methylpiperazin-1-yl)pyridine-3-carboxamide

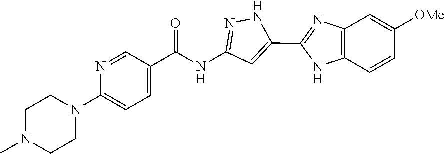 [SMILES: COc1ccc2[nH]c(-c3cc(NC(=O)c4ccc(N5CCN(C)CC5)nc4)n[nH]3)nc2c1]

A solution of N-[5-(5-methoxy-1H-benzimidazol-2-yl)-1-[(4-methoxyphenyl)methyl]pyrazol-3-yl]-6-(4-methylpiperazin-1-yl)pyridine-3-carboxamide (33 mg, 0.0597 mmol) in TFA (2 mL) was stirred at 70° C. for 1 h. The mixture was concentrated. Sat. NaHCO₃ (20 mL) was added, and the aq. phase was extracted with EtOAc (3×40 mL). The combined organic phases were washed with sat. NaHCO₃ (3×20 mL) and brine (20 mL), and concentrated. The residue was purified by reverse phase chromatography (BEH C18 30×150) eluting with water (10 mM (NH₄)(HCO₂)) and MeCN (19-29%) to provide the title compound as a solid (15.5 mg, 60%). ¹H NMR (400 MHz, DMSO) δ 13.39 (s, 1H), 12.90 (s, 1H), 10.77 (s, 1H), 8.79 (d, J=2.5 Hz, 1H), 8.32 (s, 1H), 8.14 (dd, J=9.1, 2.3 Hz, 1H), 7.60-7.11 (m, 2H), 7.01-6.80 (m, 2H), 3.81 (s, 3H), 3.67-3.60 (m, 4H), 2.42-2.37 (m, 4H), 2.22 (s, 3H). m/z (ES⁺), [M+H]⁺ 433.5. HPLC (A05) t_{R}=1.75 min.

### Preparation of Compound A-58.

### Step 1: N-(2-amino-4-methoxy-phenyl)-5-[[3-chloro-4-(3-methoxypropoxy)-benzoyl]amino]-2-[(4-methoxyphenyl)methyl]pyrazole-3-carboxamide

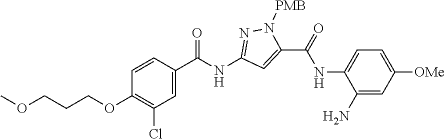

5-[[3-Chloro-4-(3-methoxypropoxy)benzoyl]amino]-2-[(4-methoxyphenyl)methyl]pyrazole-3-carboxylic acid (M-3) (200 mg, 0.422 mmol) and HATU (0.160 g, 0.422 mmol) were dissolved in DMF (5 mL) at 0° C. DIEA (0.289 mL, 1.69 mmol) was added, and the mixture was stirred at 0° C. for 5 min. 4-Methoxybenzene-1,2-diamine dihydrochloride (89.1 mg, 0.422 mmol) was added. The mixture was warmed to 23° C. and stirred for 18 h. EtOAc (200 mL) was added, and the organic phase was washed with water (4×50 mL) and brine (50 mL), dried (MgSO₄), filtered, and concentrated. The residue was purified by silica gel chromatography (12 g cartridge) eluting with hexanes and EtOAc (0-100%) to provide the title compound as a solid (189 mg, 75%). ¹H NMR (400 MHz, DMSO) δ 11.00 (s, 1H), 9.67 (s, 1H), 8.14 (d, J=2.2 Hz, 1H), 8.02 (dd, J=8.7, 2.2 Hz, 1H), 7.51 (s, 1H), 7.25 (d, J=8.8 Hz, 1H), 7.22 (d, J=8.8 Hz, 2H), 6.99 (d, J=8.6 Hz, 1H), 6.90-6.84 (m, 2H), 6.35 (d, J=2.8 Hz, 1H), 6.18 (dd, J=8.7, 2.8 Hz, 1H), 5.60 (s, 2H), 4.92 (s, 2H), 4.20 (t, J=6.3 Hz, 2H), 3.72 (s, 3H), 3.68 (s, 3H), 3.51 (t, J=6.2 Hz, 2H), 3.26-3.24 (m, 3H), 2.00 (dd, J=8.5, 3.9 Hz, 2H). m/z (ES⁺), [M+H]⁺ 595.0. HPLC (A05) t_{R}=2.54 min.

### Step 2: 3-chloro-N-[5-(5-methoxy-1H-benzimidazol-2-yl)-1-[(4-methoxyphenyl)-methyl]pyrazol-3-yl]-4-(3-methoxypropoxy)benzamide

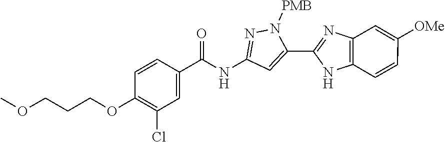

N-(2-Amino-4-methoxy-phenyl)-5-[[3-chloro-4-(3-methoxypropoxy)benzoyl]amino]-2-[(4-methoxyphenyl)methyl]pyrazole-3-carboxamide (189 mg, 0.318 mmol) was dissolved in AcOH (5 mL). The mixture was heated at 90° C. for 3 h and at 60° C. for 18 h. The mixture was concentrated. Sat. NaHCO₃ (30 mL) was added, and the aq. phase was extracted with EtOAc (3×20 mL). The combined organic phases were washed with brine (20 mL), dried (MgSO₄), filtered, and concentrated. The residue was purified by silica gel chromatography (12 g cartridge) eluting with DCM and MeOH (0-5%) to provide the title compound as a solid (121 mg, 66%). ¹H NMR (500 MHz, DMSO, 85° C.) δ 12.71 (s, 1H), 10.74 (s, 1H), 8.15 (d, J=2.2 Hz, 1H), 8.03 (dd, J=8.6, 2.2 Hz, 1H), 7.58 (s, 1H), 7.44 (s, 1H), 7.27-7.22 (m, 3H), 7.01 (s, 1H), 6.89 (s, 1H), 6.87-6.83 (m, 2H), 5.99 (s, 2H), 4.23 (t, J=6.3 Hz, 2H), 3.84 (s, 3H), 3.71 (s, 3H), 3.54 (t, J=6.3 Hz, 2H), 3.28 (s, 3H), 2.02 (p, J=6.3 Hz, 2H). m/z (ES⁺), [M+H]⁺ 577.6. HPLC (A05) t_{R}=2.66 min.

### Step 3: 3-chloro-N-[5-(5-methoxy-1H-benzimidazol-2-yl)-1H-pyrazol-3-yl]-4-(3-methoxypropoxy)benzamide

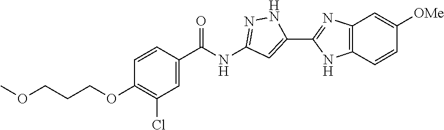 [SMILES: COCCCOc1ccc(C(=O)Nc2cc(-c3nc4cc(OC)ccc4[nH]3)[nH]n2)cc1Cl]

A solution of 3-chloro-N-[5-(5-methoxy-1H-benzimidazol-2-yl)-1-[(4-methoxyphenyl)methyl]-pyrazol-3-yl]-4-(3-methoxypropoxy)benzamide (89 mg, 0.155 mmol) in TFA (4 mL) was stirred at 23° C. for 1 h. The mixture was concentrated. The residue was dissolved in DMSO (1 mL). NEt₃ (0.500 mL) was added, and the mixture was stirred at 23° C. for 30 min. Water (25 mL) was added. The mixture was filtered, and the solid was washed with water (25 mL), acetone (25 mL), DCM (25 mL), and acetone (25 mL) and dried under high vacuum to provide a 3:1 mixture of pyrazole tautomers of the title compound as a solid (24 mg, 34%). ¹H NMR (500 MHz, DMSO) δ 13.39 [13.09] (s, 1H), 12.86 [12.58] (s, 1H), [11.14] 10.97 (s, 1H), 8.16 [8.16] (d, J=9.0 Hz, 1H), 8.04 [8.04] (d, J=7.8 Hz, 1H), 7.55 [7.55] (d, J=8.1 Hz, 1H), 7.46-7.24 [7.46-7.24] (m, 2H), 7.21-6.53 [7.21-6.53] (m, 2H), 4.22 [4.22] (t, J=6.0 Hz, 2H), 3.82 [3.82] (d, J=6.1 Hz, 3H), 3.53 [3.53] (t, J=6.2 Hz, 2H), 3.27 [3.27] (s, 3H), 2.02 [2.02] (p, J=6.2 Hz, 2H). m/z (ES⁺), [M+H]⁺ 456.5. HPLC (A05) t_{R}=2.29 min.

### Preparation of Compound A-79.

### Steps 1 and 2: 3-chloro-N-[5-(5-methoxy-1H-benzimidazol-2-yl)-1-[(4-methoxy-phenyl)methyl]pyrazol-3-yl]-4-(2-methoxyethoxy)benzamide

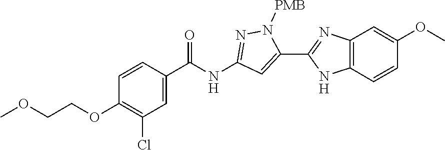

5-[[3-Chloro-4-(2-methoxyethoxy)benzoyl]amino]-2-[(4-methoxyphenyl)methyl]pyrazole-3-carboxylic acid (M-4) (110 mg, 0.239 mmol) and HATU (0.0909 g, 0.239 mmol) were dissolved in DMF (2 mL) at 0° C. DIEA (0.164 mL, 0.957 mmol) was added, and the mixture was stirred at 0° C. for 5 min. 4-Methoxybenzene-1,2-diamine dihydrochloride (50.5 mg, 0.239 mmol) was added, and the mixture was stirred at 23° C. for 18 h. The mixture was poured into water (40 mL) and filtered. The solid was dissolved in AcOH (30 mL), and the mixture was stirred at 110° C. for 3 h. After cooling to 23° C., the mixture was concentrated. The residue was dissolved in EtOAc (100 mL). The organic phase was washed with sat. NaHCO₃ (50 mL) and brine (50 mL), dried (MgSO₄), filtered, and concentrated. The residue was purified by silica gel chromatography (12 g cartridge) eluting with hexanes and EtOAc (0-70%) to provide the title compound as a solid (70.6 mg, 52%). ¹H NMR (400 MHz, DMSO) δ [13.00] 12.97 (s, 1H), 11.05 [11.05] (s, 1H), 8.15 [8.15] (d, J=2.2 Hz, 1H), 8.04 [8.04] (dd, J=8.7, 2.3 Hz, 1H), 7.60 [7.41] (d, J=8.8 Hz, 1H), 7.50 [7.49] (s, 1H), 7.29-7.20 [6.98] (m, 4H), [6.91] 6.85 (dd, J=8.9, 2.6 Hz, 3H), [6.02] 6.00 (s, 2H), 4.31-4.26 [4.31-4.26] (m, 2H), 3.83 [3.80] (s, 3H), 3.74-3.70 [3.74-3.70] (m, 2H), 3.69 [3.69] (s, 3H), 3.34 [3.34] (s, 3H). m/z (ES⁺), [M+H]⁺ 562.9. HPLC (A05) t_{R}=2.59 min.

### Step 3: 3-chloro-N-[5-(5-methoxy-1H-benzimidazol-2-yl)-1H-pyrazol-3-yl]-4-(2-methoxyethoxy)benzamide

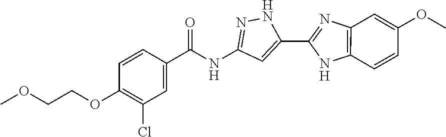

TFA (2 mL) was added to 3-chloro-N-[5-(5-methoxy-1H-benzimidazol-2-yl)-1-[(4-methoxyphenyl)methyl]pyrazol-3-yl]-4-(2-methoxyethoxy)benzamide (70 mg, 0.125 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The solid was purified by silica gel chromatography (12 g cartridge) eluting with DCM and MeOH (0-20%) to provide the title compound as as a solid in a 4:1 mixture of tautomers (27 mg, 49%). ¹H NMR (400 MHz, DMSO) δ 13.39 [13.13] (s, 1H), 12.87 [12.57] (s, 1H), [11.16] 10.97 (s, 1H), 8.15 [8.15] (s, 1H), 8.03 [8.03] (d, J=8.6 Hz, 1H), 7.52 [7.52] (s, 1H), 7.44-7.24 [7.44-7.24] (m, 2H), [7.16] 6.99 (s, 1H), 6.85 [6.85] (m, 1H), 4.33-4.25 [4.33-4.25] (m, 2H), 3.81 [3.81] (s, 3H), 3.75-3.69 [3.75-3.69] (m, 2H), 3.35 [3.35] (s, 3H). m/z (ES⁺), [M+H]⁺ 442.8. HPLC (A05) t_{R}=2.21 min.

### Preparation of Compound A-106.

### Steps 1 and 2: 3-chloro-N-[5-(7-fluoro-6-methoxy-1H-benzimidazol-2-yl)-1H-pyrazol-3-yl]-4-methoxy-benzamide

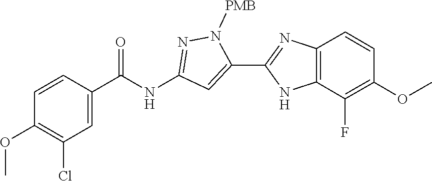

DIEA (147 μL, 0.842 mmol) was added to a solution of HATU (101 mg, 0.265 mmol) and 5-[(3-chloro-4-methoxy-benzoyl)amino]-2-[(4-methoxyphenyl)methyl]pyrazole-3-carboxylic acid (M-2) (100 mg, 0.240 mmol) in DMF (1 mL) at 0° C. The mixture was stirred at 0° C. for 15 min. 3-Fluoro-4-methoxy-benzene-1,2-diamine (P-1) (39.4 mg, 0.253 mmol) was added. The mixture was warmed to 23° C. and stirred for 3.5 h. EtOAc (15 mL) was added, and the organic phase was washed with water (15 mL), sat. NaHCO₃ (2×15 mL), and brine (15 mL). The organic phase was dried (MgSO₄), filtered, and concentrated. AcOH (2.50 mL) was added to the residue (m/z (ES⁺), [M+H]⁺ 554.7. HPLC (A05) t_{R}=2.51 min), and the mixture was heated at 100° C. for 18 h. After cooling to 23° C., the mixture was poured into water (50 mL), and the mixture was stirred at 23° C. for 5 min. The mixture was filtered. The solid was washed with sat. NaHCO₃ (50 mL) and water (50 mL) and purified by silica gel chromatography (25 g cartridge) eluting with hexanes and EtOAc (0-100%) to provide the title compound as a solid (66 mg, 51%). ¹H NMR (400 MHz, DMSO) δ 13.28 [13.46] (s, 1H), 11.11 [11.08] (s, 1H), 8.16 (d, J=2.2 Hz, 1H), 8.08 (dd, J=8.7, 2.2 Hz, 1H), 7.59-7.53 [7.70-7.62] (m, 1H), 7.34-7.22 [7.53-7.46] (m, 1H), 7.34-7.22 (m, 3H), 7.22-7.11 (m, 1H), 6.91-6.83 (m, 2H), 6.01 (s, 2H), 3.95 (s, 3H), 3.90 (s, 3H), 3.70 (s, 3H). ¹⁹F NMR (376 MHz, DMSO) δ −151.30 [−151.98] (d, J=6.8 Hz). m/z (ES⁺), [M+H]⁺ 536.6. HPLC (A05) t_{R}=2.63 min.

### Step 3: 3-chloro-N-[5-(7-fluoro-6-methoxy-1H-benzimidazol-2-yl)-1H-pyrazol-3-yl]-4-methoxy-benzamide

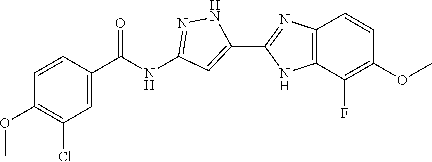 [SMILES: COc1ccc(C(=O)Nc2cc(-c3nc4ccc(OC)c(F)c4[nH]3)[nH]n2)cc1Cl]

TFA (1 mL) was added to 3-chloro-N-[5-(7-fluoro-6-methoxy-1H-benzimidazol-2-yl)-1-[(4-methoxyphenyl)methyl]pyrazol-3-yl]-4-methoxy-benzamide (63.5 mg, 0.118 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL) and purified by silica gel chromatography (4 g cartridge) eluting with DCM and MeOH (0-10%) to provide the title compound as a solid (27.6 mg, 56%). ¹H NMR (500 MHz, DMSO) δ 13.54 (s, 1H), 13.21 (s, 1H), 11.01 (s, 1H), 8.26-7.98 (m, 2H), 7.60-6.98 (m, 4H), 3.97 (s, 3H), 3.90 (s, 3H). ¹⁹F NMR (376 MHz, DMSO) δ −151.01 [−152.18]. m/z (ES⁺), [M+H]⁺ 416.6. HPLC (A05) t_{R}=2.26 min.

### Preparation of Compound A-127.

### Steps 1 and 2: 3-chloro-N-[5-(7-fluoro-5-methoxy-1H-benzimidazol-2-yl)-1-[(4-methoxyphenyl)methyl]pyrazol-3-yl]-4-methoxy-benzamide

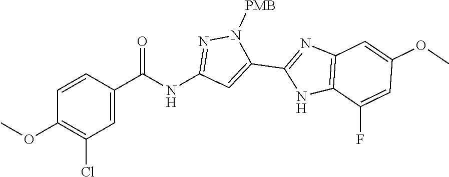

DIEA (126 μL, 0.721 mmol) was added to a solution of HATU (96 mg, 0.253 mmol) and 5-[(3-chloro-4-methoxy-benzoyl)amino]-2-[(4-methoxyphenyl)methyl]pyrazole-3-carboxylic acid (M-2) (100 mg, 0.240 mmol) in DMF (2 mL) at 0° C. The mixture was stirred at 0° C. for 10 min. 3-Fluoro-5-methoxy-benzene-1,2-diamine (27.3 mg, 0.175 mmol) was added.

The mixture was warmed to 23° C. and stirred for 5 h. The mixture was poured into water (30 mL) and stirred at 23° C. for 5 min. The mixture was filtered, and the solid was dissolved in EtOAc (50 mL). The organic phase was dried (MgSO₄), filtered, and concentrated. AcOH (2 mL) was added to the residue (m/z (ES⁺), [M+H]⁺ 554.6. HPLC (A05) t_{R}=2.50 [2.56] min), and the mixture was heated at 100° C. for 18 h. After cooling to 23° C., the mixture was poured into water (30 mL) and stirred at 23° C. for 5 min. The mixture was filtered, and the solid was washed with sat. NaHCO₃ (30 mL) and water (50 mL) and dissolved in EtOAc (50 mL). The organic phase was dried (MgSO₄), filtered, and concentrated. The residue was purified by silica gel chromatography (25 g cartridge) eluting with hexanes and EtOAc (0-100%) to provide the title compound as a solid in a 6:1 mixture of benzimidazole tautomers (40.6 mg, 32%). ¹H NMR (400 MHz, DMSO) δ 13.27 [13.49] (s, 1H), 11.10 [11.08] (s, 1H), 8.16 (d, J=2.2 Hz, 1H), 8.08 (dd, J=8.7, 2.2 Hz, 1H), 7.53 [7.65] (s, 1H), 7.30-7.12 (m, 3H), 6.91-6.72 (m, 4H), 5.98 [6.01] (s, 2H), 3.95 (s, 3H), 3.85 [3.82] (s, 3H), 3.70 (s, 3H). 19F NMR (376 MHz, DMSO) δ −127.19 [−128.35] (d, J=12.4 Hz). m/z (ES⁺), [M+H]⁺ 536.6. HPLC (A05) t_{R}=2.67 min.

### Step 3: 3-chloro-N-[5-(7-fluoro-5-methoxy-1H-benzimidazol-2-yl)-1H-pyrazol-3-yl]-4-methoxy-benzamide

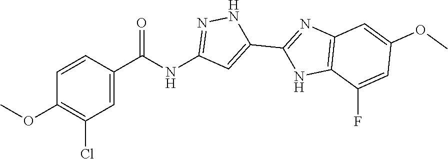

TFA (1 mL) was added to 3-chloro-N-[5-(7-fluoro-5-methoxy-1H-benzimidazol-2-yl)-1-[(4-methoxyphenyl)methyl]pyrazol-3-yl]-4-methoxy-benzamide (38.8 mg, 0.0724 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL) and dried under high vacuum to provide the title compound as a solid in a 3:1 mixture of pyrazole tautomers (25.1 mg, 83%). ¹H NMR (500 MHz, DMSO) δ 13.52 (s, 1H), 13.17 (s, 1H), 11.01 (s, 1H), 8.21-8.11 (m, 1H), 8.11-7.95 (m, 1H), 7.56-7.37 (m, 1H), 7.37-7.17 (m, 1H), 6.98-6.78 (m, 1H), 6.76-6.52 (m, 1H), 3.96 (s, 3H), 3.83 (s, 3H). ¹⁹F NMR (471 MHz, DMSO) δ −126.93 [−127.55]. m/z (ES⁺), [M+H]⁺ 416.5. HPLC (A05) t_{R}=2.30 min.

### EXAMPLE B21

### Preparation of Compounds A-38, A-57, and A-72

### Representative Scheme for Synthesis of Compounds A-38, A-57, and A-72.

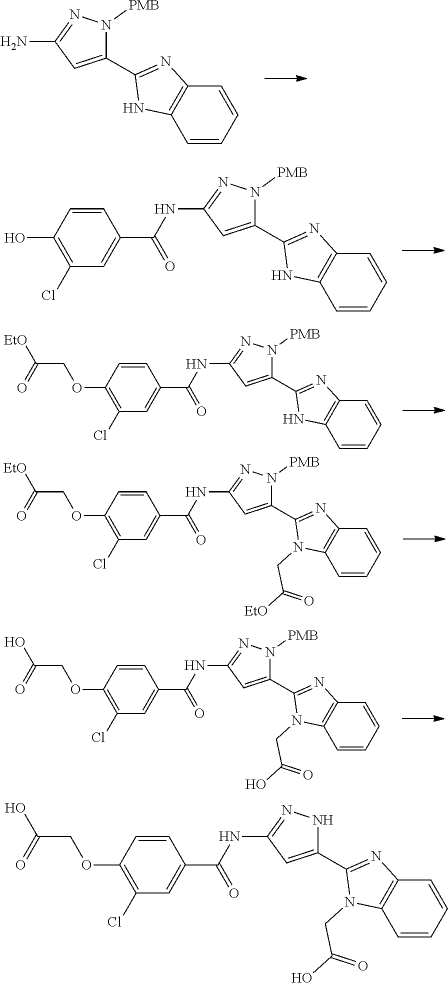

### Preparation of Compound A-38.

### Step 1: N-[5-(1H-benzimidazol-2-yl)-1-[(4-methoxyphenyl)methyl]-pyrazol-3-yl]-3-chloro-4-hydroxy-benzamide

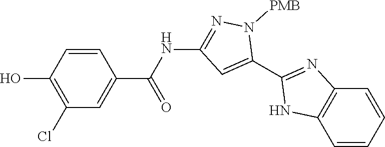

3-Chloro-4-hydroxy-benzoic acid (150 mg, 0.869 mmol) and HATU (0.331 g, 0.869 mmol) were dissolved in DMF (7 mL) at 0° C. DIEA (0.298 mL, 1.74 mmol) was added, and the mixture was stirred at 0° C. for 5 min. 5-(1H-Benzimidazol-2-yl)-1-[(4-methoxyphenyl)-methyl]pyrazol-3-amine (J-1) (278 mg, 0.869 mmol) was added. The mixture was warmed to 23° C., stirred for 18 h, and concentrated. Water (50 mL) was added, and the aq. phase was extracted with EtOAc (3×50 mL). The combined organic phases were washed with brine (60 mL), dried (MgSO₄), filtered, and concentrated. The product was purified by silica gel chromatography (40 g cartridge) eluting with hexanes and EtOAc (0-100%) to provide the title compound as a solid (60 mg, 15%). ¹H NMR (400 MHz, DMSO) δ 13.12 (s, 1H), 10.96 (s, 1H), 10.91 (s, 1H), 8.10 (d, J=2.2 Hz, 1H), 7.89 (dd, J=8.5, 2.2 Hz, 1H), 7.73-7.70 (m, 1H), 7.54 (td, J=3.3, 0.9 Hz, 2H), 7.26-7.22 (m, 4H), 7.03 (d, J=8.5 Hz, 1H), 6.87-6.83 (m, 2H), 6.03 (s, 2H), 3.68 (s, 3H). m/z (ES⁺), [M+H]⁺ 474.4. HPLC (A05) t_{R}=2.46 min.

### Step 2: ethyl 2-[4-[[5-(1H-benzimidazol-2-yl)-1-[(4-methoxyphenyl)-methyl]pyrazol-3-yl]carbamoyl]-2-chloro-phenoxy]acetate and ethyl 2-[2-[5-[[3-chloro-4-(2-ethoxy-2-oxo-ethoxy)benzoyl]amino]-2-[(4-methoxyphenyl)methyl]pyrazol-3-yl]benzimidazol-1-yl]acetate

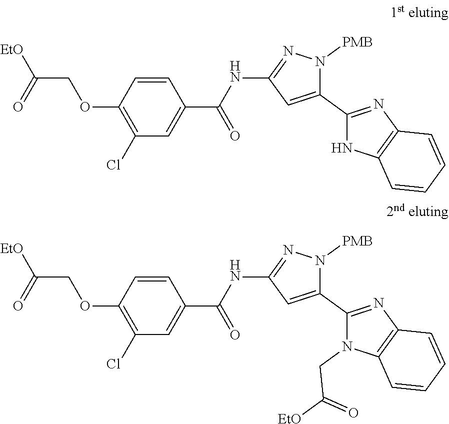

Ethyl 2-bromoacetate (18.3 μL, 0.165 mmol) was added to a solution of N-[5-(1H-benzimidazol-2-yl)-1-[(4-methoxyphenyl)methyl]pyrazol-3-yl]-3-chloro-4-hydroxy-benzamide (60 mg, 0.127 mmol) and K₂CO₃ (35 mg, 0.253 mmol) in DMF (2 mL). The solution was stirred at 100° C. for 18 h. Water (50 mL) was added, and the aq. phase was extracted with EtOAc (3×50 mL). The combined organic phases were washed with water (50 mL) and brine (30 mL), dried (MgSO₄), filtered, and concentrated. The residue was purified by silica gel chromatography (12 g) eluting with hexanes and EtOAc (0-60%) to provide two compounds. 1^{st} eluting compound: ethyl 2-[4-[[5-(1H-benzimidazol-2-yl)-1-[(4-methoxyphenyl)-methyl]pyrazol-3-yl]carbamoyl]-2-chloro-phenoxy]acetate as a solid (15 mg, 21%). ¹H NMR (400 MHz, DMSO) δ 13.14 (s, 1H), 11.10 (s, 1H), 8.17 (d, J=2.2 Hz, 1H), 8.00 (dd, J=8.7, 2.2 Hz, 1H), 7.72 (d, J=7.7 Hz, 1H), 7.55 (s, 1H), 7.34-7.21 (m, 5H), 7.18 (d, J=8.8 Hz, 1H), 6.87-6.84 (m, 2H), 6.04 (s, 2H), 5.03 (s, 2H), 4.21-4.16 (m, 2H), 3.68 (s, 3H), 1.23 (td, J=4.8, 2.4 Hz, 3H). m/z (ES⁺), [M+H]⁺ 560.6. HPLC (A05) t_{R}=2.65 min. 2^{nd} eluting compound: ethyl 2-[2-[5-[[3-chloro-4-(2-ethoxy-2-oxo-ethoxy)benzoyl]amino]-2-[(4-methoxyphenyl)methyl]pyrazol-3-yl]benzimidazol-1-yl]acetate as a solid (40 mg, 49%). ¹H NMR (400 MHz, DMSO) δ 11.12 (s, 1H), 8.15 (d, J=2.3 Hz, 1H), 7.98 (dd, J=8.7, 2.2 Hz, 1H), 7.82-7.78 (m, 1H), 7.72-7.69 (m, 1H), 7.39-7.31 (m, 2H), 7.24-7.16 (m, 3H), 7.08 (s, 1H), 6.87-6.83 (m, 2H), 5.57 (s, 2H), 5.26 (s, 2H), 5.02 (s, 2H), 4.24-4.15 (m, 4H), 3.70 (s, 3H), 1.25-1.20 (m, 6H). m/z (ES⁺), [M+H]⁺ 647.0. HPLC (A05) t_{R}=2.67 min.

### Step 3: 2-[2-[5-[[4-(carboxymethoxy)-3-chloro-benzoyl]amino]-2-[(4-methoxyphenyl)methyl]pyrazol-3-yl]benzimidazol-1-yl]acetic acid

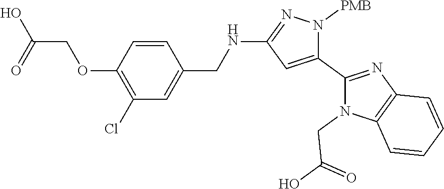

Ethyl 2-[2-[5-[[3-chloro-4-(2-ethoxy-2-oxo-ethoxy)benzoyl]amino]-2-[(4-methoxyphenyl)-methyl]pyrazol-3-yl]benzimidazol-1-yl]acetate (40 mg, 0.0619 mmol) was dissolved in THF (0.500 mL) and MeOH (0.500 mL). 1.00 M aq. NaOH (0.310 mL, 0.310 mmol) was added. The mixture was heated at 60° C., and stirred for 20 min. The mixture was concentrated. Water (5 mL) was added and acidified to pH 6 with 1M aq. HCl. The solid was filtered and dried under high vacuum to provide the title compound as a solid (36 mg, 99%). ¹H NMR (400 MHz, DMSO) δ 11.10 (s, 1H), 8.15 (d, J=2.3 Hz, 1H), 7.98 (dd, J=8.6, 2.3 Hz, 1H), 7.79 (d, J=7.1 Hz, 1H), 7.69 (d, J=7.4 Hz, 1H), 7.38-7.30 (m, 2H), 7.19 (d, J=8.8 Hz, 2H), 7.12 (dd, J=16.4, 8.0 Hz, 2H), 6.84 (d, J=8.8 Hz, 2H), 5.57 (s, 2H), 5.14 (s, 2H), 4.92 (s, 2H), 3.70 (s, 3H). m/z (ES), [M+H]⁺: 591.6. HPLC (A05) t_{R}=1.95 min.

### Step 4: 2-[2-[3-[[4-(carboxymethoxy)-3-chloro-benzoyl]amino]-1H-pyrazol-5-yl]benzimidazol-1-yl]acetic acid

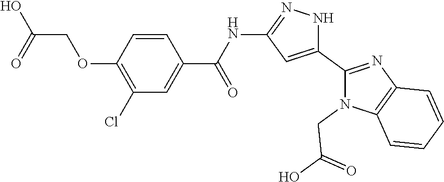 [SMILES: O=C(O)COc1ccc(C(=O)Nc2cc(-c3nc4ccccc4n3CC(=O)O)[nH]n2)cc1Cl]

A solution of 2-[2-[5-[[4-(carboxymethoxy)-3-chloro-benzoyl]amino]-2-[(4-methoxyphenyl)-methyl]pyrazol-3-yl]benzimidazol-1-yl]acetic acid (38 mg, 0.0644 mmol) in TFA (3 mL) was heated to 70° C. for 1 h. The mixture was concentrated. Sat. NaHCO₃ (10 mL) and brine (10 mL) were added, and the suspension was stirred for 10 min. The mixture was filtered, and the solid was purified by reverse phase chromatography (BEH C18 30×150) eluting with water (10 mM (NH₄)(HCO₂)) and MeCN (15-25%) to provide the title compound as a solid (8.3 mg, 27%). ¹H NMR (400 MHz, DMSO) δ 8.29 (s, 2H), 8.13 (d, J=1.8 Hz, 1H), 7.92 (d, J=7.6 Hz, 1H), 7.68-7.62 (m, 1H), 7.48 (d, J=7.0 Hz, 1H), 7.26-7.18 (m, 2H), 7.03 (s, 1H), 6.95 (d, J=8.6 Hz, 1H), 5.03 (s, 2H), 4.49 (s, 2H). m/z (ES+), [M+H]⁺ 470.4. HPLC (A05) t_{R}=1.70 min.

### Preparation of Compound A-57 (ethyl 2-[4-[[5-(1H-benzimidazol-2-yl)-1H-pyrazol-3-yl]carbamoyl]-2-chloro-phenoxy]acetate)

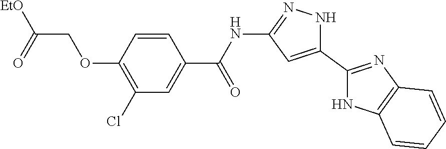 [SMILES: CCOC(=O)COc1ccc(C(=O)Nc2cc(-c3nc4ccccc4[nH]3)[nH]n2)cc1Cl]

A solution of ethyl 2-[4-[[5-(1H-benzimidazol-2-yl)-1-[(4-methoxyphenyl)methyl]pyrazol-3-yl]carbamoyl]-2-chloro-phenoxy]acetate (A-38, Step 2) (37 mg, 0.0661 mmol) in TFA (2 mL) was stirred at 23° C. for 1 h. The mixture was concentrated. The residue was dissolved in DMSO (0.500 mL). NEt₃ (0.500 mL) was added, and the mixture was stirred at 23° C. for 5 min. Water (25 mL) was added. The mixture was filtered, and the solid was washed with water (25 mL), acetone (25 mL), DCM (25 mL), and acetone (25 mL) and dried under high vacuum to provide a 3:1 mixture of pyrazole tautomers of the title compound as a solid (27 mg, 93%). ¹H NMR (400 MHz, DMSO) δ 13.51 [13.16] (s, 1H), 13.03 [12.74] (s, 1H), [11.16] 11.01 (s, 1H), 8.17 [8.17] (s, 1H), 8.01 [8.01] (d, J=8.4 Hz, 1H), 7.67 [7.67] (d, J=7.2 Hz, 1H), 7.53 [7.53] (d, J=7.3 Hz, 1H), 7.42 [7.42] (s, 1H), 7.22 [7.22] (m, 3H), 5.04 [5.04] (s, 2H), 4.20 [4.20] (q, J=7.1 Hz, 2H), 1.23 [1.23] (t, J=7.1 Hz, 3H). m/z (ES⁺), [M+H]⁺ 440.8. HPLC (A05) t_{R}=2.28 min.

### Preparation of Compound A-72 (2-[4-[[5-(1H-benzimidazol-2-yl)-1H-pyrazol-3-yl]carbamoyl]-2-chloro-phenoxy]acetic acid)

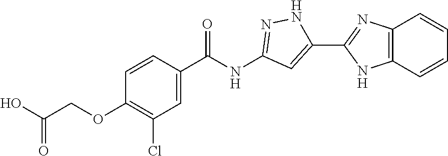 [SMILES: O=C(O)COc1ccc(C(=O)Nc2cc(-c3nc4ccccc4[nH]3)[nH]n2)cc1Cl]

Ethyl 2-[4-[[5-(1H-benzimidazol-2-yl)-1H-pyrazol-3-yl]carbamoyl]-2-chloro-phenoxy]acetate (A-57) (17 mg, 0.0386 mmol) was dissolved in THF (0.400 mL) and MeOH (0.400 mL). 1.00 M aq. NaOH (0.386 mL, 0.386 mmol) was added. The mixture was heated at 60° C. and stirred for 1 h. The mixture was partially concentrated, and water (10 mL) was added. Aq. HCl (1 M) was added dropwise until the pH was 2-3, at which point a precipitate formed. The mixture was lyophilized, and the residue was purified by reverse phase chromatography (BEH C18 30×150) eluting with water (10 mM (NH₄)(HCO₃)) and MeCN (17-37%) to provide the title compound as a solid (10.6 mg, 67%). ¹H NMR (500 MHz, DMSO) δ 8.20 (s, 1H), 8.14 (s, 1H), 7.92 (s, 1H), 7.63 (s, 1H), 7.48 (s, 1H), 7.19 (s, 2H), 6.99 (d, J=9.2 Hz, 1H), 4.55 (s, 2H). m/z (ES⁺), [M+H]⁺ 412.3. HPLC (A05) t_{R}=1.85 min.

### EXAMPLE B22

### Preparation of Compounds A-62 and A-70

### Representative Scheme for the Synthesis of Compounds A-62 and A-70.

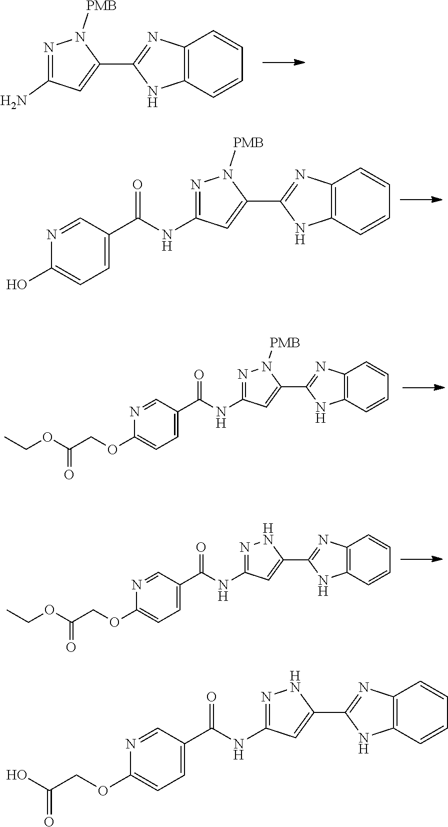

### Preparation of Compound A-70.

### Step 1: N-[5-(1H-benzimidazol-2-yl)-1-[(4-methoxyphenyl)methyl]pyrazol-3-yl]-6-hydroxy-pyridine-3-carboxamide

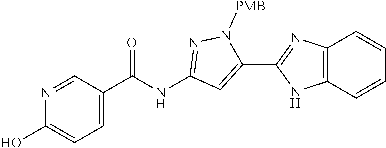

6-Hydroxypyridine-3-carboxylic acid (131 mg, 0.939 mmol), HATU (0.357 g, 0.939 mmol), and 5-(1H-benzimidazol-2-yl)-1-[(4-methoxyphenyl)methyl]pyrazol-3-amine (J-1) (300 mg, 0.939 mmol) were dissolved in DMF (5 mL) at 0° C. DIEA (3.22 mL, 18.8 mmol) was added. The mixture was warmed to 23° C. and stirred for 18 h. EtOAc (150 mL) was added, and the organic phase was washed with water (4×50 mL) and brine (50 mL), dried (MgSO₄), filtered, and concentrated. The residue was purified by silica gel chromatography (25 g cartridge) eluting with DCM and MeOH (0-20%) to provide the title compound as a solid (275 mg, 66%). ¹H NMR (400 MHz, DMSO) δ 13.12 (s, 1H), 12.05 (s, 1H), 10.88 (s, 1H), 8.26 (s, 1H), 8.01 (dd, J=9.7, 2.7 Hz, 1H), 7.72 (d, J=7.4 Hz, 1H), 7.56-7.51 (m, 1H), 7.50 (s, 1H), 7.31-7.18 (m, 4H), 6.88-6.82 (m, 2H), 6.37 (d, J=9.7 Hz, 1H), 6.02 (s, 2H), 3.68 (s, 3H). m/z (ES⁺), [M+H]⁺ 441.8. HPLC (A05) t_{R}=2.16 min.

### Step 2: ethyl 2-[[5-[[5-(1H-benzimidazol-2-yl)-1-[(4-methoxyphenyl)-methyl]pyrazol-3-yl]carbamoyl]-2-pyridyl]oxy]acetate

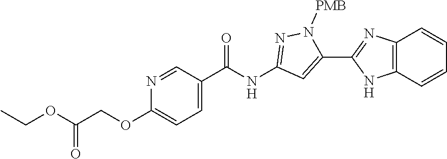

Ethyl 2-bromoacetate (30.2 μL, 0.272 mmol) was added to a solution of N-[5-(1H-benzimidazol-2-yl)-1-[(4-methoxyphenyl)methyl]pyrazol-3-yl]-6-hydroxy-pyridine-3-carboxamide (100 mg, 0.227 mmol) and NEt₃ (63.3 μL, 0.454 mmol) in DMF (3.50 mL). The mixture was stirred at 80° C. for 2 h. After cooling to 23° C., EtOAc (150 mL) was added. The organic phase was washed with water (4×50 mL) and brine (50 mL), dried (MgSO₄), filtered, and concentrated. The residue was purified by silica gel chromatography (12 g cartridge) eluting with DCM and MeOH (0-10%) to provide the title compound as a solid (41 mg, 34%). ¹H NMR (400 MHz, DMSO) δ 13.14 (s, 1H), 10.90 (s, 1H), 8.58 (d, J=2.4 Hz, 1H), 8.10 (dd, J=9.6, 2.6 Hz, 1H), 7.72 (d, J=8.2 Hz, 1H), 7.54 (d, J=8.2 Hz, 1H), 7.50 (s, 1H), 7.31-7.19 (m, 4H), 6.87-6.82 (m, 2H), 6.47 (d, J=9.5 Hz, 1H), 6.02 (s, 2H), 4.78 (s, 2H), 4.16 (q, J=7.1 Hz, 2H), 3.68 (s, 3H), 1.22 (t, J=7.1 Hz, 3H). m/z (ES⁺), [M+H]⁺ 528.0. HPLC (A05) t_{R}=2.36 min.

### Step 3: ethyl 2-[[5-[[5-(1H-benzimidazol-2-yl)-1H-pyrazol-3-yl]carbamoyl]-2-pyridyl]oxy]acetate

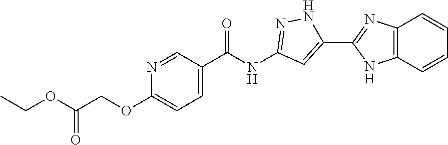 [SMILES: CCOC(=O)COc1ccc(C(=O)Nc2cc(-c3nc4ccccc4[nH]3)[nH]n2)cn1]

TFA (2 mL) was added to ethyl 2-[[5-[[5-(1H-benzimidazol-2-yl)-1-[(4-methoxyphenyl)methyl]pyrazol-3-yl]carbamoyl]-2-pyridyl]oxy]acetate (40 mg, 0.0760 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The solid was purified by silica gel chromatography (12 g cartridge) eluting with DCM and MeOH (0-20%) to provide the title compound as a solid (8.20 mg, 27%). ¹H NMR (400 MHz, DMSO) δ 13.45 (s, 1H), 12.99 (s, 1H), 10.79 (s, 1H), 8.54 (d, J=2.1 Hz, 1H), 8.07 (d, J=9.7 Hz, 1H), 7.62 (s, 1H), 7.48 (s, 1H), 7.33 (s, 1H), 7.19 (s, 2H), 6.45 (d, J=8.6 Hz, 1H), 4.75 (s, 2H), 4.13 (q, J=7.1 Hz, 2H), 1.18 (t, J=7.1 Hz, 3H). m/z (ES⁺), [M+H]⁺ 407.5. HPLC (A05) t_{R}=1.95 min.

### Preparation of Compound A-62.

### Step 1: 2-[[5-[[5-(1H-benzimidazol-2-yl)-1H-pyrazol-3-yl]carbamoyl]-2-pyridyl]-oxy]acetic acid

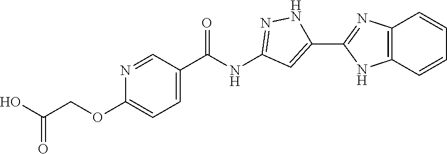 [SMILES: O=C(O)COc1ccc(C(=O)Nc2cc(-c3nc4ccccc4[nH]3)[nH]n2)cn1]

Ethyl 2-[[5-[[5-(1H-benzimidazol-2-yl)-1H-pyrazol-3-yl]carbamoyl]-2-pyridyl]oxy]acetate (A-70) (15 mg, 0.0369 mmol) was dissolved in THF (0.400 mL) and MeOH (0.400 mL). 2.50 M aq. NaOH (78.3 μL, 0.185 mmol) was added. The mixture was stirred at 60° C. for 1 h. The mixture was concentrated. Water (10 mL) was added. Aq. HCl (1 M) was added dropwise until the pH was 2-3, at which point a precipitate formed. The mixture was filtered, and the solid was purified by prep HPLC (BEH C18 30×150) eluting with water (10 mM (NH₄)(HCO₃)) and MeCN (10-30%) to provide the title compound as a solid (4.9 mg, 35%). ¹H NMR (500 MHz, DMSO) δ 13.49 (s, 1H), 13.08 (s, 1H), 10.77 (s, 1H), 8.53 (s, 1H), 8.18 (s, 1H), 8.03 (d, J=8.7 Hz, 1H), 7.58 (m 2H), 7.37 (s, 1H), 7.21 (s, 2H), 6.42 (d, J=9.6 Hz, 1H), 4.48 (s, 2H). m/z (ES-), [M−H]⁻ 377.4. HPLC (A05) t_{R}=1.68 min.

### EXAMPLE B23

### Preparation of Compounds A-71, A-74, and A-76

### Representative Scheme for the Synthesis of Compounds A-71, A-74, and A-76.

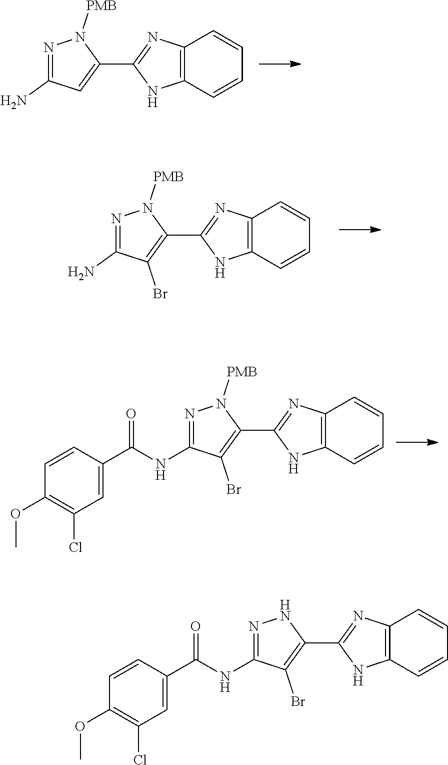

### Preparation of Compound A-71.

### Step 1: 5-(1H-benzimidazol-2-yl)-4-bromo-1-[(4-methoxyphenyl)methyl]-pyrazol-3-amine

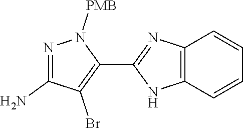

A solution of 5-(1H-benzimidazol-2-yl)-1-[(4-methoxyphenyl)methyl]pyrazol-3-amine (J-1) (500 mg, 1.57 mmol) and NBS (0.334 g, 1.88 mmol) in DCM (40 mL) was stirred at 23° C. for 18 h. DCM (100 mL) was added, and the organic phase was washed with water (60 mL), sat. NaHCO₃ (60 mL), and brine (60 mL). The organic phase was dried (MgSO₄), filtered, and concentrated. The residue was purified by silica gel chromatography (25 g cartridge) eluting with hexanes and EtOAc (0-100%) to provide the title compound as a solid (534 mg, 86%). ¹H NMR (400 MHz, DMSO) δ 12.68 (s, 1H), 7.75 (d, J=8.1 Hz, 1H), 7.60 (d, J=7.7 Hz, 1H), 7.32-7.23 (m, 2H), 7.06-7.01 (m, 2H), 6.82-6.76 (m, 2H), 5.42 (s, 2H), 5.05 (s, 2H), 3.67 (s, 3H). m/z (ES⁺), [M+H]⁺ 399.4. HPLC (A05) t_{R}=2.24 min.

### Step 2: N-[5-(1H-benzimidazol-2-yl)-4-bromo-1-[(4-methoxyphenyl)methyl]-pyrazol-3-yl]-3-chloro-4-methoxy-benzamide

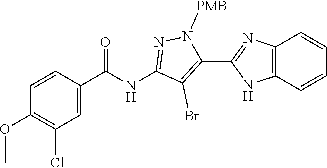

3-Chloro-4-methoxy-benzoic acid (24.4 mg, 0.131 mmol) and HATU (49.6 mg, 0.131 mmol) were dissolved in DMF (2 mL) at 0° C. DIEA (44.7 μL, 0.261 mmol) was added, and the mixture was stirred at 0° C. for 5 min. 5-(1H-Benzimidazol-2-yl)-4-bromo-1-[(4-methoxyphenyl)-methyl]pyrazol-3-amine (52 mg, 0.131 mmol) was added. The mixture was warmed to 23° C. and stirred for 18 h. EtOAc (80 mL) was added, and the organic phase was washed with water (3×30 mL) and brine (50 mL), dried (MgSO₄), filtered, and concentrated. The residue was purified by silica gel chromatography (12 g cartridge) eluting with hexanes and EtOAc (0-70%) to provide a mixture of the title compound and starting material as a solid that was used without further purification (3:4 ratio, 44 mg). ¹H NMR (500 MHz, DMSO) δ 12.94 (s, 1H), 10.37 (s, 1H), 8.08 (d, J=2.2 Hz, 1H), 8.01 (dd, J=8.6, 2.3 Hz, 1H), 7.80 (d, J=7.0 Hz, 1H), 7.62 (d, J=7.9 Hz, 1H), 7.35-7.28 (m, 4H), 7.13 (d, J=8.8 Hz, 2H), 6.83 (d, J=8.8 Hz, 2H), 5.65 (s, 2H), 5.04 (s, 2H), 3.69 (s, 3H). m/z (ES⁺), [M+H]⁺ 567.4. HPLC (A05) t_{R}=2.53 min.

### Step 3: N-[5-(1H-benzimidazol-2-yl)-4-bromo-1H-pyrazol-3-yl]-3-chloro-4-methoxy-benzamide

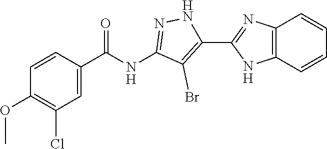 [SMILES: COc1ccc(C(=O)Nc2n[nH]c(-c3nc4ccccc4[nH]3)c2Br)cc1Cl]

TFA (2 mL) was added to N-[5-(1H-benzimidazol-2-yl)-4-bromo-1-[(4-methoxyphenyl)methyl]pyrazol-3-yl]-3-chloro-4-methoxy-benzamide (74 mg, 0.131 mmol), and the mixture was stirred at 23° C. for 1 h. The mixture was concentrated. The residue was purified by silica gel chromatography (12 g cartridge) eluting with DCM and MeOH (0-10%) to provide the title compound as a solid (5.60 mg, 10% over two steps). ¹H NMR (400 MHz, DMSO) δ 10.41 (s, 1H), 8.08 (d, J=2.1 Hz, 1H), 8.03-7.95 (m, 1H), 7.61 (s, 2H), 7.30 (d, J=8.8 Hz, 1H), 7.23 (s, 2H), 3.93 (s, 3H). m/z (ES⁺), [M+H]⁺ 448.2. HPLC (A05) t_{R}=2.22 min.

### Preparation of Compound A-74.

### Step 1: 5-(1H-benzimidazol-2-yl)-4-chloro-1-[(4-methoxyphenyl)methyl]pyrazol-3-amine

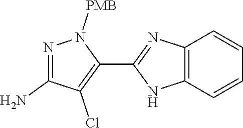

A solution of 5-(1H-benzimidazol-2-yl)-1-[(4-methoxyphenyl)methyl]pyrazol-3-amine (J-1) (500 mg, 1.57 mmol) and NCS (0.230 g, 1.72 mmol) in DCM (30 mL) was stirred at 23° C. for 18 h. DCM (100 mL) was added, and the organic phase was washed with sat. NaHCO₃ (3×50 mL), water (50 mL), and brine (50 mL). The organic phase was dried (MgSO₄), filtered, and concentrated. The residue was purified by silica gel chromatography (25 g cartridge) eluting with hexanes and EtOAc (0-100%) to provide the title compound as a solid (414 mg, 75%). ¹H NMR (400 MHz, DMSO) δ 12.63 (s, 1H), 7.74 (d, J=7.2 Hz, 1H), 7.61 (d, J=7.3 Hz, 1H), 7.33-7.20 (m, 2H), 7.10-7.02 (m, 2H), 6.82-6.76 (m, 2H), 5.51 (s, 2H), 5.12 (s, 2H), 3.67 (s, 3H). m/z (ES⁺), [M+H]⁺ 354.7. HPLC (A05) t_{R}=2.26 min.

### Step 2: N-[5-(1H-benzimidazol-2-yl)-4-chloro-1-[(4-methoxyphenyl)methyl]-pyrazol-3-yl]-3-chloro-4-methoxy-benzamide

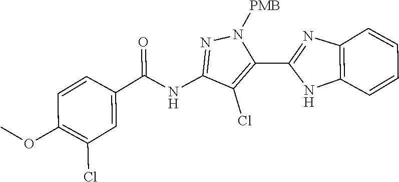

Oxalyl chloride (0.0574 mL, 0.678 mmol) was added dropwise at 23° C. to a mixture of 3-chloro-4-methoxy-benzoic acid (63.3 mg, 0.339 mmol) and DMF (1 drop) in DCM (7 mL). The mixture was stirred at 23° C. for 1.5 h and concentrated. The residue was dissolved in THF (4 mL), and pyridine (0.0686 mL, 0.848 mmol) was added dropwise. The mixture was stirred at 23° C. for 10 min. 5-(1H-Benzimidazol-2-yl)-4-chloro-1-[(4-methoxyphenyl)methyl]pyrazol-3-amine (100 mg, 0.283 mmol) was added. The mixture was stirred for 1 h and concentrated. The residue was purified by silica gel chromatography (12 g cartridge) eluting with hexanes and EtOAc (0-70%) to provide the title compound as a solid (82 mg, 56%). ¹H NMR (400 MHz, DMSO) δ 12.92 (s, 1H), 10.44 (s, 1H), 8.09 (d, J=2.2 Hz, 1H), 8.01 (dd, J=8.7, 2.2 Hz, 1H), 7.80 (d, J=7.5 Hz, 1H), 7.63 (d, J=7.3 Hz, 1H), 7.36-7.26 (m, 3H), 7.20-7.14 (m, 2H), 6.86-6.80 (m, 2H), 5.74 (s, 2H), 3.95 (s, 3H), 3.69 (s, 3H). m/z (ES+), [M+H]⁺ 523.4. HPLC (A05) t_{R}=2.55 min.

### Step 3: N-[5-(1H-benzimidazol-2-yl)-4-chloro-1H-pyrazol-3-yl]-3-chloro-4-methoxy-benzamide

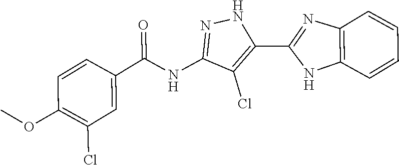 [SMILES: COc1ccc(C(=O)Nc2n[nH]c(-c3nc4ccccc4[nH]3)c2Cl)cc1Cl]

TFA (2 mL) was added to N-[5-(1H-benzimidazol-2-yl)-4-chloro-1-[(4-methoxyphenyl)methyl]pyrazol-3-yl]-3-chloro-4-methoxy-benzamide (74 mg, 0.142 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The residue was purified by silica gel chromatography (12 g cartridge) eluting with DCM and MeOH (0-10%) to provide the title compound as a solid (40 mg, 70%). ¹H NMR (400 MHz, DMSO) δ 13.94 (s, 1H), 12.73 (s, 1H), 10.48 (s, 1H), 8.13 (d, J=2.1 Hz, 1H), 8.04 (dd, J=8.7, 2.2 Hz, 1H), 7.70 (s, 1H), 7.57 (s, 1H), 7.33 (d, J=8.8 Hz, 1H), 7.24 (s, 2H), 3.97 (s, 3H). m/z (ES⁺), [M+H]⁺ 403.3. HPLC (A05) t_{R}=2.21 min.

### Preparation of Compound A-76.

### Step 1: 5-(1H-benzimidazol-2-yl)-4-fluoro-1-[(4-methoxyphenyl)methyl]pyrazol-3-amine

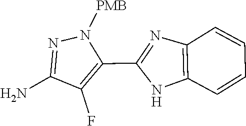

A solution of 5-(1H-benzimidazol-2-yl)-1-[(4-methoxyphenyl)methyl]pyrazol-3-amine (J-1) (200 mg, 0.626 mmol) and Selectfluor (0.244 g, 0.689 mmol) in DCM (8 mL) was stirred at 23° C. for 18 h. MeCN (2 mL) was added, and the solution was stirred for an additional 2 h. DCM (100 mL) was added, and the organic phase was washed with water (60 mL), sat. NaHCO₃ (60 mL), and brine (60 mL). The organic phase was dried (MgSO₄), filtered, and concentrated. The residue was purified by silica gel chromatography (25 g cartridge) eluting with hexanes and EtOAc (0-100%) to provide the title compound as a solid (40 mg, 19%). ¹H NMR (400 MHz, DMSO) δ 12.58 (s, 1H), 7.72 (d, J=7.6 Hz, 1H), 7.57 (d, J=6.9 Hz, 1H), 7.25 (ddd, J=9.2, 7.5, 1.3 Hz, 2H), 7.13-7.08 (m, 2H), 6.82-6.78 (m, 2H), 5.61 (s, 2H), 5.07 (s, 2H), 3.67 (s, 3H). m/z (ES⁺), [M+H]⁺ 338.2. HPLC (A05) t_{R}=2.36 min.

### Step 2: N-[5-(1H-benzimidazol-2-yl)-4-fluoro-1-[(4-methoxyphenyl)methyl]-pyrazol-3-yl]-3-chloro-4-methoxy-benzamide

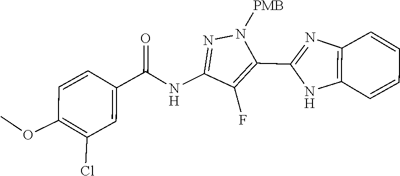

Oxalyl chloride (0.0482 mL, 0.569 mmol) was added dropwise at 23° C. to a mixture of 3-chloro-4-methoxy-benzoic acid (26.5 mg, 0.142 mmol) and DMF (0.914 μL, 0.0119 mmol) in DCM (2 mL). The mixture was stirred at 23° C. for 2 h and concentrated. The residue was dissolved in THF (1.60 mL), and pyridine (0.0288 mL, 0.356 mmol) was added dropwise. The mixture was stirred at 23° C. for 10 min. 5-(1H-Benzimidazol-2-yl)-4-fluoro-1-[(4-methoxyphenyl)-methyl]pyrazol-3-amine (40 mg, 0.119 mmol) was added. The mixture was stirred for 18 h and concentrated. The residue was purified by silica gel chromatography (25 g cartridge) eluting with hexanes and EtOAc (0-100%) to provide the title compound as a solid, which was used as such without further purification (29.3 mg, 49%). ¹H NMR (400 MHz, DMSO) δ 12.88 (s, 1H), 10.61 (s, 1H), 8.11 (d, J=2.3 Hz, 1H), 7.89 (dd, J=6.7, 1.9 Hz, 2H), 7.78 (d, J=7.9 Hz, 1H), 7.30 (dd, J=9.1, 1.5 Hz, 2H), 7.23 (dd, J=9.0, 2.4 Hz, 3H), 6.85 (d, J=8.8 Hz, 2H), 5.85 (s, 2H), 3.93 (s, 3H), 3.69 (s, 3H). m/z (ES⁺), [M+H]⁺ 506.4. HPLC (A05) t_{R}=2.57 min.

### Step 3: N-[5-(1H-benzimidazol-2-yl)-4-fluoro-1H-pyrazol-3-yl]-3-chloro-4-methoxy-benzamide

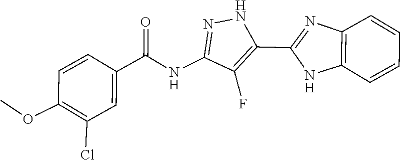 [SMILES: COc1ccc(C(=O)Nc2n[nH]c(-c3nc4ccccc4[nH]3)c2F)cc1Cl]

TFA (2 mL) was added to N-[5-(1H-benzimidazol-2-yl)-4-fluoro-1-[(4-methoxyphenyl)methyl]pyrazol-3-yl]-3-chloro-4-methoxy-benzamide (29.3 mg, 0.0579 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL). The solid was purified by preparative HPLC (BEH C18 30×150) eluting with water (10 mM (NH₄)(HCO₃)) and MeCN (34-54%) to provide the title compound as a solid (8.80 mg, 39%). ¹H NMR (400 MHz, DMSO) δ 8.13 (d, J=2.2 Hz, 1H), 8.04 (dd, J=8.7, 2.2 Hz, 1H), 7.62 (s, 2H), 7.32 (d, J=8.8 Hz, 1H), 7.23 (dd, J=5.9, 3.1 Hz, 2H), 3.96 (s, 3H). m/z (ES⁺), [M+H]⁺ 386.2. HPLC (A05) t_{R}=2.17 min.

### EXAMPLE B24

### Preparation of Compounds A-78, A-93, A-112, A-161, A-162, and A-163

### Representative Scheme for Synthesis of Compounds A-112, A-161, A-162, and A-163.

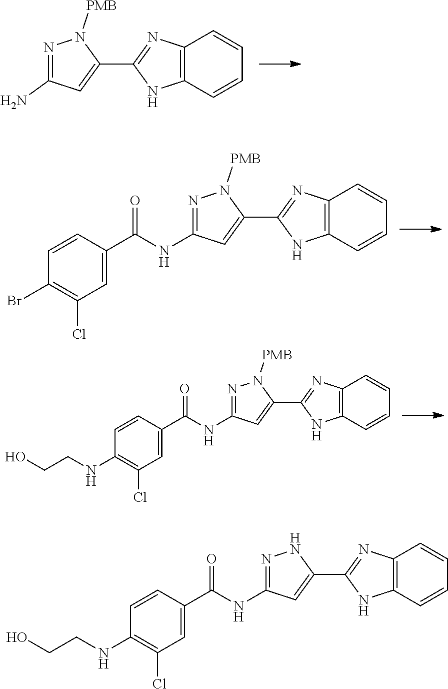

### Preparation of Compound A-78.

### Step 1: N-[5-(1H-benzimidazol-2-yl)-1-[(4-methoxyphenyl)methyl]pyrazol-3-yl]-4-bromo-3-chloro-benzamide

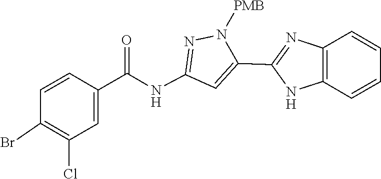

4-Bromo-3-chloro-benzoic acid (485 mg, 2.06 mmol) and HATU (0.783 g, 2.06 mmol) were dissolved in DMF (10 mL) at 0° C. DIEA (0.705 mL, 4.12 mmol) was added, and the mixture was stirred at 0° C. for 5 min. 5-(1H-Benzimidazol-2-yl)-1-[(4-methoxyphenyl)methyl]pyrazol-3-amine (J-1) (538 mg, 1.69 mmol) was added, the mixture was stirred at 23° C. for 18 h. EtOAc (100 mL) was added, and the organic phase was washed with water (3×50 mL) and brine (60 mL), dried (MgSO₄), filtered, and concentrated. The residue was purified by silica gel chromatography (40 g cartridge) eluting with hexanes and EtOAc (0-50%) to provide the title compound as a solid (837 mg, 76%). ¹H NMR (400 MHz, DMSO) δ 13.15 (s, 1H), 11.33 (s, 1H), 8.27 (d, J=1.5 Hz, 1H), 7.95-7.87 (m, 2H), 7.72 (d, J=7.9 Hz, 1H), 7.56 (s, 1H), 7.56-7.53 (m, 1H), 7.31-7.20 (m, 4H), 6.88-6.83 (m, 2H), 6.04 (s, 2H), 3.68 (s, 3H). m/z (ES⁺), [M+H]⁺ 537.3. HPLC (A05) t_{R}=2.76 min.

### Step 2: N-[5-(1H-benzimidazol-2-yl)-1-[(4-methoxyphenyl)methyl]pyrazol-3-yl]-3-chloro-4-(2-hydroxyethylamino)benzamide

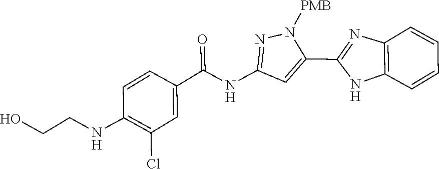

Nitrogen was bubbled through a solution of Pd(OAc)₂ (4.18 mg, 0.0186 mmol) and Xantphos (10.8 mg, 0.0186 mmol) in DMF (2 mL). The mixture was heated to 70° C. for 10 min. N-[5-(1H-Benzimidazol-2-yl)-1-[(4-methoxyphenyl)methyl]pyrazol-3-yl]-4-bromo-3-chloro-benzamide (100 mg, 0.186 mmol), 2-aminoethanol (0.0281 mL, 0.466 mmol), and Cs₂CO₃ (0.121 g, 0.373 mmol) were added. The mixture was stirred at 120° C. for 18 h. After cooling to 23° C., water (50 mL) was added, and the aq. phase was extracted with EtOAc (4×50 mL). The combined organic phases were washed with brine (100 mL), dried (MgSO₄), filtered, and concentrated. The residue was purified by silica gel chromatography (12 g cartridge) eluting with DCM and MeOH (0-5%) to provide the title compound as a solid (24.6 mg, 26%). ¹H NMR (400 MHz, DMSO) δ 13.11 (s, 1H), 10.79 (s, 1H), 8.03 (d, J=2.1 Hz, 1H), 7.92 (dd, J=8.4, 2.1 Hz, 1H), 7.71 (d, J=7.9 Hz, 1H), 7.55-7.50 (m, 2H), 7.30-7.20 (m, 4H), 6.87-6.83 (m, 2H), 6.80 (d, J=8.9 Hz, 1H), 6.02 (s, 2H), 5.81 (t, J=5.8 Hz, 1H), 4.85 (t, J=5.6 Hz, 1H), 3.68 (s, 3H), 3.59 (q, J=5.7 Hz, 2H), 3.29 (t, J=2.9 Hz, 2H). m/z (ES⁺), [M+H]⁺ 517.5. HPLC (A05) t_{R}=2.42 min.

### Step 3: N-[5-(1H-benzimidazol-2-yl)-1H-pyrazol-3-yl]-3-chloro-4-(2-hydroxy-ethylamino)benzamide

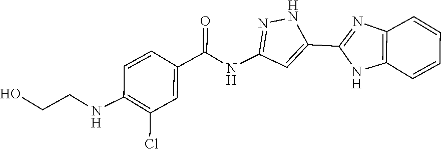 [SMILES: O=C(Nc1cc(-c2nc3ccccc3[nH]2)[nH]n1)c1ccc(NCCO)c(Cl)c1]

TFA (2 mL) was added to N-[5-(1H-benzimidazol-2-yl)-1-[(4-methoxyphenyl)methyl]pyrazol-3-yl]-3-chloro-4-(2-hydroxyethylamino)benzamide (24.6 mg, 0.0476 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The solid was purified by preparative HPLC (BEH C18 30×150) eluting with water (10 mM (NH₄)(HCO₂)) and MeCN (30-40%) to provide the title compound as a solid (5.70 mg, 30%). ¹H NMR (400 MHz, DMSO) δ 13.36 (s, 1H), 12.77 (s, 1H), 10.69 (s, 1H), 8.31 (s, 1H), 7.98 (d, J=1.9 Hz, 1H), 7.87 (dd, J=8.6, 1.7 Hz, 1H), 7.63-7.30 (m, 2H), 7.15 (s, 2H), 6.80 (d, J=8.7 Hz, 1H), 5.80 (s,1H), 4.81 (s, 1H), 3.56 (t, J=5.9 Hz, 2H), 3.26 (dd, J=11.3, 5.4 Hz, 2H). m/z (ES⁺), [M+H]⁺ 397.3. HPLC (A05) t_{R}=2.02 min.

### Preparation of Compound A-93.

### Step 1: N-[5-(1H-benzimidazol-2-yl)-1-[(4-methoxyphenyl)methyl]pyrazol-3-yl]-3-chloro-4-(2-methoxyethylsulfanyl)benzamide

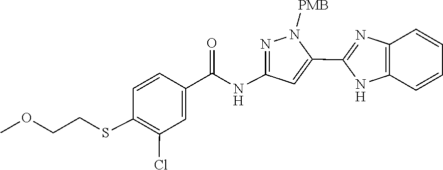

Nitrogen was bubbled through a solution of Pd(OAc)₂ (0.00753 g, 0.0335 mmol) and tBu-Brettphos (0.0217 g, 0.0447 mmol) in DMF (2.40 mL). N-[5-(1H-Benzimidazol-2-yl)-1-[(4-methoxyphenyl)methyl]pyrazol-3-yl]-4-bromo-3-chloro-benzamide (A-78, Step 1) (120 mg, 0.224 mmol), 2-methoxyethanethiol (0.0305 mL, 0.335 mmol), and NEt₃ (0.0312 mL, 0.224 mmol) were added. The mixture was heated at 140° C. for 24 h. After cooling to 23° C., water (50 mL) was added, and the aq. phase was extracted with EtOAc (4×50 mL). The combined organic phases were washed with brine (100 mL), dried (MgSO₄), filtered, and concentrated. The residue was purified by silica gel chromatography (12 g cartridge) eluting with hexanes and EtOAc (0-100%) to provide the title compound as a solid (15 mg, 12%). ¹H NMR (400 MHz, DMSO) δ 13.14 (s, 1H), 11.18 (s, 1H), 8.13 (d, J=1.9 Hz, 1H), 8.00 (dd, J=8.3, 1.9 Hz, 1H), 7.72 (d, J=9.6 Hz, 1H), 7.58-7.49 (m, 3H), 7.30-7.20 (m, 4H), 6.88-6.83 (m, 2H), 6.04 (s, 2H), 3.69 (s, 3H), 3.63 (t, J=6.2 Hz, 2H), 3.30-3.29 (m, 5H). m/z (ES⁺), [M+H]⁺ 548.8. HPLC (A05) t_{R}=2.69 min.

### Step 2: N-[5-(1H-benzimidazol-2-yl)-1H-pyrazol-3-yl]-3-chloro-4-(2-methoxyethylsulfanyl)benzamide

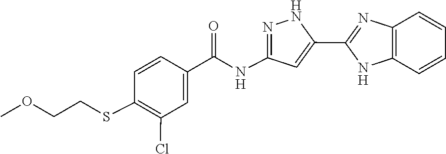 [SMILES: COCCSc1ccc(C(=O)Nc2cc(-c3nc4ccccc4[nH]3)[nH]n2)cc1Cl]

TFA (1 mL) was added to N-[5-(1H-benzimidazol-2-yl)-1-[(4-methoxyphenyl)methyl]pyrazol-3-yl]-3-chloro-4-(2-methoxyethylsulfanyl)benzamide (15 mg, 0.0274 mmol), and the mixture was stirred at 23° C. for 1 h. The mixture was concentrated. The residue was dissolved in DMSO (0.400 mL). NEt₃ (0.800 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL) to provide the title compound as a solid in a 6:1 mixture of tautomers (5.4 mg, 46%). ¹H NMR (500 MHz, DMSO) δ 13.52 [13.19] (s, 1H), 13.03 [12.73] (s, 1H), [11.24] 11.10 (s, 1H), 8.15-8.07 (m, 1H), 8.03-7.94 (m, 1H), 7.70-7.58 (m, 1H), 7.58-7.46 (m, 2H), 7.42 [6.68] (s, 1H), 7.28-7.12 (m, 2H), 3.64 (t, J=6.2 Hz, 2H), 3.35-3.26 (m, 5H). m/z (ES⁺), [M+H]⁺ 428.3. HPLC (A05) t_{R}=2.31 min.

### EXAMPLE B25

### Preparation of Compounds A-112, A-161, A-162, and A-163

Representative Scheme for Synthesis of compounds A-112, A-161, A-162, and A-163.

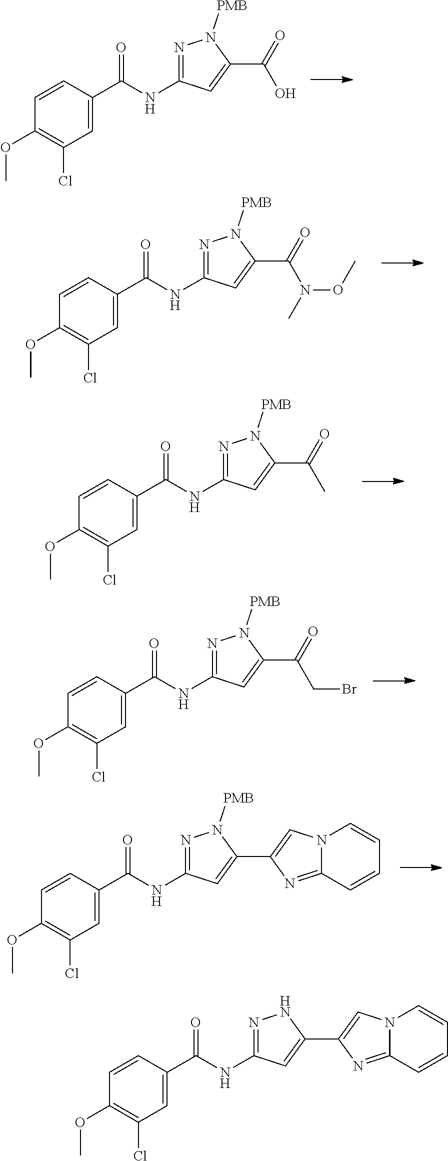

### Preparation of Compound A-112.

### Step 1: 5-[(3-chloro-4-methoxy-benzoyl)amino]-N-methoxy-2-[(4-methoxy-phenyl)methyl]-N-methyl-pyrazole-3-carboxamide

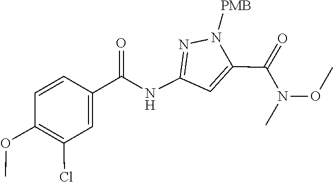

DIEA (0.733 mL, 4.21 mmol) was added to a solution of HATU (0.503 g, 1.32 mmol) and 5-[(3-chloro-4-methoxy-benzoyl)amino]-2-[(4-methoxyphenyl)methyl]pyrazole-3-carboxylic acid (M-2) (0.500 g, 1.20 mmol) in DMF (5 mL) at 0° C. The mixture was stirred at 0° C. for 5 min. N-Methoxymethanamine hydrochloride (0.129 g, 1.32 mmol) was added. The mixture was warmed to 23° C. and stirred for 18 h. Water (50 mL) was added, and the mixture was stirred at 23° C. for 10 min. The mixture was filtered, and the solid was washed with sat. NaHCO₃ (25 mL) and water (20 mL) and purified by silica gel chromatography (25 g cartridge) eluting with hexanes and EtOAc (0-100%) to provide the title compound as a solid (416 mg, 75%). ¹H NMR (500 MHz, DMSO) δ 11.02 (s, 1H), 8.14 (d, J=2.2 Hz, 1H), 8.04 (dd, J=8.7, 2.2 Hz, 1H), 7.26 (d, J=8.8 Hz, 1H), 7.18-7.14 (m, 2H), 7.13 (s, 1H), 6.92-6.85 (m, 2H), 5.45 (s, 2H), 3.94 (s, 3H), 3.72 (s, 3H), 3.58 (s, 3H), 3.28 (s, 3H). m/z (ES⁺), [M+H]⁺ 459.6. HPLC (A05) t_{R}=2.43 min.

### Step 2: N-[5-acetyl-1-[(4-methoxyphenyl)methyl]pyrazol-3-yl]-3-chloro-4-methoxy-benzamide

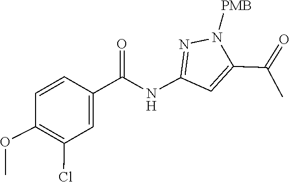

3.0M MeMgBr in Et₂O (652 μL, 1.95 mmol) was added dropwise at 0° C. to a solution of 5-[(3-chloro-4-methoxy-benzoyl)amino]-N-methoxy-2-[(4-methoxyphenyl)methyl]-N-methyl-pyrazole-3-carboxamide (299 mg, 0.652 mmol) in THF (6 mL). The mixture was warmed to 23° C. and stirred for 1 h. The mixture was cooled to 0° C., and 1N HCl (20 mL) was added dropwise. The mixture was stirred at 0° C. for 5 min. The aq. phase was extracted with EtOAc (2×20 mL). The combined organic phases were washed with sat. NaHCO₃ (20 mL) and brine (20 mL), dried (MgSO₄), filtered, and concentrated. The residue was purified by silica gel chromatography (25 g cartridge) eluting with hexanes and EtOAc (0-100%) to provide the title compound as a solid (177 mg, 66%). ¹H NMR (500 MHz, DMSO) δ 11.10 (s, 1H), 8.15 (d, J=2.2 Hz, 1H), 8.05 (dd, J=8.7, 2.3 Hz, 1H), 7.43 (s, 1H), 7.27 (d, J=8.8 Hz, 1H), 7.20-7.09 (m, 2H), 6.92-6.84 (m, 2H), 5.57 (s, 2H), 3.95 (s, 3H), 3.72 (s, 3H), 2.55 (s, 3H). m/z (ES⁺), [M−H]⁻ 412.6. HPLC (A05) t_{R}=2.54 min.

### Step 3: N-[5-(2-bromoacetyl)-1-[(4-methoxyphenyl)methyl]pyrazol-3-yl]-3-chloro-4-methoxy-benzamide

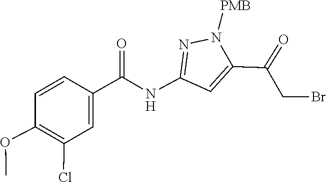

N-[5-Acetyl-1-[(4-methoxyphenyl)methyl]pyrazol-3-yl]-3-chloro-4-methoxy-benzamide (40 mg, 0.0967 mmol) and DIEA (42.1 μL, 0.242 mmol) were dissolved in DCM (1 mL), and the mixture was cooled to 0° C. TMSOTf (43.7 μL, 0.242 mmol) was added. The mixture was warmed to 23° C. and stirred for 1.5 h. The mixture was cooled to 0° C. A solution of NBS (21.5 mg, 0.121 mmol) in DCM (0.500 mL) was added dropwise, and the mixture was stirred at 0° C. for 1 h. The mixture was concentrated. Sat. NaHCO₃ (10 mL) was added, and the aq. phase was extracted with EtOAc (3×10 mL). The combined organic phases were washed with brine (10 mL), dried (MgSO₄), filtered, and concentrated to provide the title compound as a solid (37 mg, 78%). ¹H NMR (400 MHz, DMSO) δ 11.15 (s, 1H), 8.15 (d, J=2.2 Hz, 1H), 8.05 (dd, J=8.7, 2.2 Hz, 1H), 7.57 (s, 1H), 7.28 (d, J=8.8 Hz, 1H), 7.19-7.14 (m, 2H), 6.92-6.87 (m, 2H), 5.57 (s, 2H), 4.84 (s, 2H), 3.95 (s, 3H), 3.73 (s, 3H). m/z (ES⁺), [M−H]⁻ 490.4. HPLC (A05) t_{R}=2.62 min.

### Steps 4: 3-chloro-N-[5-imidazo[1,2-a]pyridin-2-yl-1-[(4-methoxyphenyl)methyl]-pyrazol-3-yl]-4-methoxy-benzamide

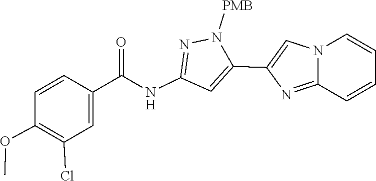

N-[5-(2-Bromoacetyl)-1-[(4-methoxyphenyl)methyl]pyrazol-3-yl]-3-chloro-4-methoxy-benzamide (37 mg, 0.0751 mmol) was dissolved in EtOH (1 mL). K₂CO₃ (20.8 mg, 0.150 mmol) and pyridin-2-amine (9.19 mg, 0.0976 mmol) were added. The mixture was stirred at 80° C. for 3 h. After cooling to 23° C., the mixture was concentrated. Water (10 mL) was added, the aq. phase was extracted with EtOAc (3×10 mL). The combined organic phases were washed with brine (10 mL), dried (MgSO₄), filtered, and concentrated. The residue was purified by silica gel chromatography (25 g cartridge) eluting with hexanes and EtOAc (0-100%) to provide the title compound as a solid (16.4 mg, 45%). ¹H NMR (500 MHz, DMSO) δ 10.93 (s, 1H), 8.56 (dt, J=6.8, 1.2 Hz, 1H), 8.40 (s, 1H), 8.15 (d, J=2.2 Hz, 1H), 8.06 (dd, J=8.7, 2.2 Hz, 1H), 7.65 (d, J=9.1 Hz, 1H), 7.32 (ddd, J=9.1, 6.7, 1.2 Hz, 1H), 7.26 (d, J=8.8 Hz, 1H), 7.20-7.15 (m, 2H), 7.08 (s, 1H), 6.98 (td, J=6.8, 1.1 Hz, 1H), 6.87-6.82 (m, 2H), 5.86 (s, 2H), 3.94 (s, 3H), 3.69 (s, 3H). m/z (ES⁺), [M+H]⁺ 488.6. HPLC (A05) t_{R}=2.49 min.

### Step 5: 3-chloro-N-(5-imidazo[1,2-a]pyridin-2-yl-1H-pyrazol-3-yl)-4-methoxy-benzamide

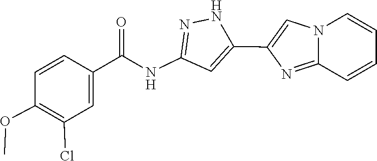 [SMILES: COc1ccc(C(=O)Nc2cc(-c3cn4ccccc4n3)[nH]n2)cc1Cl]

TFA (1 mL) was added to 3-chloro-N-[5-imidazo[1,2-a]pyridin-2-yl-1-[(4-methoxyphenyl)methyl]pyrazol-3-yl]-4-methoxy-benzamide (40 mg, 0.0820 mmol), and the mixture was heated at 50° C. for 1.5 h. After cooling to 23° C., the mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with sat. NaHCO₃ (10 mL) and water (10 mL). The solid was purified by silica gel chromatography (25 g cartridge) eluting with DCM and MeOH (0-10%) to provide the title compound as a solid (16.3 mg, 54%). ¹H NMR (400 MHz, DMSO) δ 13.03 (s, 1H), 10.85 (s, 1H), 8.60 (d, J=6.7 Hz, 1H), 8.31 (s, 1H), 8.16 (d, J=2.1 Hz, 1H), 8.09-8.03 (m, 1H), 7.59 (d, J=9.1 Hz, 1H), 7.33-7.24 (m, 2H), 7.04 (s, 1H), 6.94 (t, J=6.4 Hz, 1H), 3.95 (s, 3H). m/z (ES⁺), [M+H]⁺ 368.5. HPLC (A05) t_{R}=2.09 min.

### Preparation of Compound A-161.

### Step 1: 3-chloro-4-methoxy-N-[5-(6-methoxyimidazo[1,2-a]pyridin-2-yl)-1-[(4-methoxyphenyl)methyl]pyrazol-3-yl]benzamide

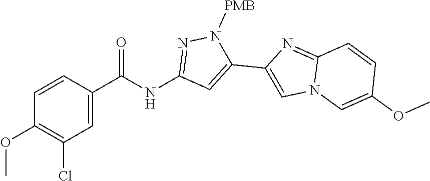

N-[5-(2-Bromoacetyl)-1-[(4-methoxyphenyl)methyl]pyrazol-3-yl]-3-chloro-4-methoxy-benzamide (A-112, Step 3) (50 mg, 0.101 mmol) was dissolved in EtOH (2.50 mL). K₂CO₃ (28 mg, 0.203 mmol) and 5-methoxypyridin-2-amine (16.4 mg, 0.132 mmol) were added. The mixture was stirred at 80° C. for 2 h, cooled to 23° C., and concentrated. Water (10 mL) was added, and the aq. phase was extracted with EtOAc (3×5 mL). The combined organic phases were washed with brine (15 mL), dried (MgSO₄), filtered, and concentrated. The residue was purified by silica gel chromatography (12 g cartridge) eluting with hexanes and EtOAc (10-100%) to provide the title compound as an oil (27 mg, 51%). ¹H NMR (400 MHz, DMSO) δ 10.86 (s, 1H), 8.20 (dd, J=3.6, 1.1 Hz, 2H), 8.09 (d, J=2.2 Hz, 1H), 8.00 (dd, J=8.7, 2.2 Hz, 1H), 7.51 (d, J=9.7 Hz, 1H), 7.20 (d, J=8.9 Hz, 1H), 7.11 (dd, J=6.9, 4.8 Hz, 2H), 7.05 (dd, J=9.7, 2.4 Hz, 1H), 6.97 (s, 1H), 6.82-6.76 (m, 2H), 5.77 (s, 2H), 3.88 (s, 3H), 3.76 (s, 3H), 3.64 (s, 3H), 3.27 (s, 2H). m/z (ES), [M−H]⁻ 516.0. HPLC (A05) t_{R}=2.55 min.

### Step 2: 3-chloro-4-methoxy-N-[5-(6-methoxyimidazo[1,2-a]pyridin-2-yl)-1H-pyrazol-3-yl]benzamide

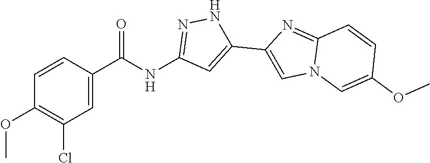 [SMILES: COc1ccc2nc(-c3cc(NC(=O)c4ccc(OC)c(Cl)c4)n[nH]3)cn2c1]

TFA (1 mL) was added to 3-chloro-4-methoxy-N-[5-(6-methoxyimidazo[1,2-a]pyridin-2-yl)-1-[(4-methoxyphenyl)methyl]pyrazol-3-yl]benzamide (27 mg, 0.0520 mmol), and the mixture was heated at 50° C. for 1.5 h. After cooling to 23° C., the mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with sat. NaHCO₃ (10 mL) and water (10 mL). The solid was purified by preparative HPLC (BEH C18 30×150) eluting with water (10 mM (NH₄)(HCO₃)) and MeCN (37-47%) to provide the title compound as a solid (7.00 mg, 34%). ¹H NMR (400 MHz, DMSO) δ 12.99-12.95 (m, 1H), 10.85-10.82 (m, 1H), 8.30 (s, 1H), 8.20 (s, 1H), 8.15 (s, 1H), 8.05 (s, 1H), 7.49 (s, 1H), 7.26 (s, 1H), 7.09-7.05 (m, 1H), 6.99 (s, 1H), 3.94 (s, 3H), 3.81 (s, 3H), 3.29 (s, 2H). m/z (ES⁺), [M−H]⁻ 396.7. HPLC (A05) t_{R}=2.17 min.

### Preparation of Compound A-162.

### Step 1: 3-chloro-4-methoxy-N-[5-(7-methoxyimidazo[1,2-a]pyridin-2-yl)-1-[(4-methoxyphenyl)methyl]pyrazol-3-yl]benzamide

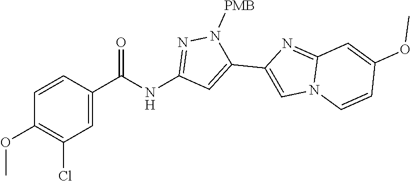

N-[5-(2-Bromoacetyl)-1-[(4-methoxyphenyl)methyl]pyrazol-3-yl]-3-chloro-4-methoxy-benzamide (A-112, Step 3) (50 mg, 0.101 mmol) was dissolved in EtOH (2.50 mL). K₂CO₃ (28 mg, 0.203 mmol) and 4-methoxypyridin-2-amine (16.4 mg, 0.132 mmol) were added. The mixture was stirred at 80° C. for 2 h, cooled to 23° C., and concentrated. Water (10 mL) was added, and the aq. phase was extracted with EtOAc (3×5 mL). The combined organic phases were washed with brine (5 mL), dried (MgSO₄), filtered, and concentrated. The residue was purified by silica gel chromatography (12 g cartridge) eluting with hexanes and EtOAc (40-100%) to provide the title compound as a solid (32 mg, 61%). ¹H NMR (400 MHz, DMSO) δ 10.89 (s, 1H), 8.40 (d, J=7.4 Hz, 1H), 8.18 (s, 1H), 8.13 (d, J=2.2 Hz, 1H), 8.04 (dd, J=8.7, 2.3 Hz, 1H), 7.25 (d, J=8.8 Hz, 1H), 7.17 (d, J=8.7 Hz, 2H), 7.02 (d, J=2.4 Hz, 1H), 7.00 (s, 1H), 6.85 (d, J=8.7 Hz, 2H), 6.68 (dd, J=7.4, 2.5 Hz, 1H), 5.83 (s, 2H), 3.93 (s, 3H), 3.85 (s, 3H), 3.69 (s, 3H). m/z (ES⁺), [M−H]⁻ 516.4. HPLC (A05) t_{R}=2.49 min.

### Step 2: 3-chloro-4-methoxy-N-[5-(7-methoxyimidazo[1,2-a]pyridin-2-yl)-1H-pyrazol-3-yl]benzamide

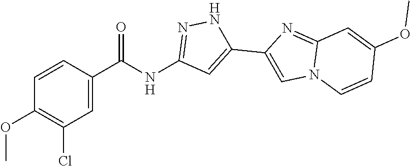 [SMILES: COc1ccn2cc(-c3cc(NC(=O)c4ccc(OC)c(Cl)c4)n[nH]3)nc2c1]

TFA (1 mL) was added to 3-chloro-4-methoxy-N-[5-(7-methoxyimidazo[1,2-a]pyridin-2-yl)-1-[(4-methoxyphenyl)methyl]pyrazol-3-yl]benzamide (27 mg, 0.0520 mmol), and the mixture was heated at 50° C. for 1.5 h. After cooling to 23° C., the mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with sat. NaHCO₃ (10 mL) and water (10 mL). The solid was purified by silica gel chromatography (7 g cartridge) eluting with DCM and MeOH (0-10%) to provide the title compound as a solid (10 mg, 48%). ¹H NMR (400 MHz, DMSO) δ 12.91 (s, 1H), 10.82 (s, 1H), 8.44 (d, J=7.4 Hz, 1H), 8.12 (m, 2H), 8.05 (d, J=8.8 Hz, 1H), 7.27 (d, J=8.7 Hz, 1H), 6.97 (s, 1H), 6.92 (s, 1H), 6.65 (d, J=5.3 Hz, 1H), 3.94 (s, 3H), 3.85 (s, 3H). m/z (ES⁻), [M−H]⁻ 396.4. HPLC (A05) t_{R}=2.09 min.

### Preparation of Compound A-163.

### Step 1: 3-chloro-4-methoxy-N-[1-[(4-methoxyphenyl)methyl]-5-(7-methylimidazo[1,2-a]pyridin-2-yl)pyrazol-3-yl]benzamide

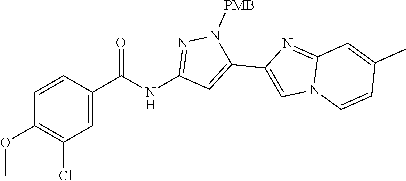

N-[5-(2-Bromoacetyl)-1-[(4-methoxyphenyl)methyl]pyrazol-3-yl]-3-chloro-4-methoxy-benzamide (A-112, Step 3) (50 mg, 0.101 mmol) was dissolved in EtOH (2.50 mL). K₂CO₃ (28 mg, 0.203 mmol) and 4-methylpyridin-2-amine (14.3 mg, 0.132 mmol) were added. The mixture was stirred at 80° C. for 2.5 h, cooled to 23° C., and concentrated. Water (10 mL) was added, and the aq. phase was extracted with EtOAc (3×10 mL). The combined organic phases were washed with brine (10 mL), dried (MgSO₄), filtered, and concentrated. The residue was purified by silica gel chromatography (12 g cartridge) eluting with hexanes and EtOAc (25-100%) to provide the title compound as a solid (22.2 mg, 44%). ¹H NMR (500 MHz, DMSO) δ 10.91 (s, 1H), 8.44 (dd, J=6.8, 1.0 Hz, 1H), 8.29 (d, J=0.7 Hz, 1H), 8.15 (d, J=2.2 Hz, 1H), 8.05 (dd, J=8.7, 2.3 Hz, 1H), 7.45-7.41 (m, 1H), 7.26 (d, J=8.8 Hz, 1H), 7.21-7.15 (m, 2H), 7.04 (s, 1H), 6.88-6.80 (m, 3H), 5.84 (s, 2H), 3.94 (s, 3H), 3.69 (s, 3H), 2.38 (s, 3H). m/z (ES⁺), [M+H]⁺ 502.6. HPLC (A05) t_{R}=2.56 min.

### Step 2: 3-chloro-4-methoxy-N-[5-(7-methylimidazo[1,2-a]pyridin-2-yl)-1H-pyrazol-3-yl]benzamide

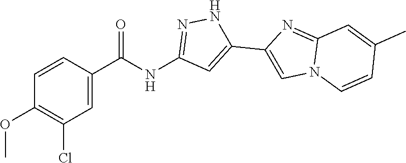 [SMILES: COc1ccc(C(=O)Nc2cc(-c3cn4ccc(C)cc4n3)[nH]n2)cc1Cl]

TFA (1 mL) was added to 3-chloro-4-methoxy-N-[1-[(4-methoxyphenyl)methyl]-5-(7-methylimidazo[1,2-a]pyridin-2-yl)pyrazol-3-yl]benzamide (24 mg, 0.0478 mmol), and the mixture was heated at 50° C. for 1.5 h. After cooling to 23° C., the mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with sat. NaHCO₃ (10 mL) and water (10 mL). The solid was purified by silica gel chromatography (7 g cartridge) eluting with DCM and MeOH (2-10%) to provide the title compound as a solid (4 mg, 22%). ¹H NMR (400 MHz, DMSO) δ 12.98 (s, 1H), 10.83 (s, 1H), 8.47 (d, J=6.7 Hz, 1H), 8.20 (s, 1H), 8.15 (s, 1H), 8.06 (d, J=8.5 Hz, 1H), 7.35 (s, 1H), 7.27 (d, J=8.6 Hz, 1H), 6.99 (s, 1H), 6.78 (d, J=7.1 Hz, 1H), 3.94 (s, 3H), 2.37 (s, 3H). m/z (ES⁺), [M−H]⁻ 380.7. HPLC (A05) t_{R}=2.13 min.

### EXAMPLE B26

### Preparation of Compounds A-130 and A-141

### Representative Scheme for Synthesis of Compounds A-130 and A-141.

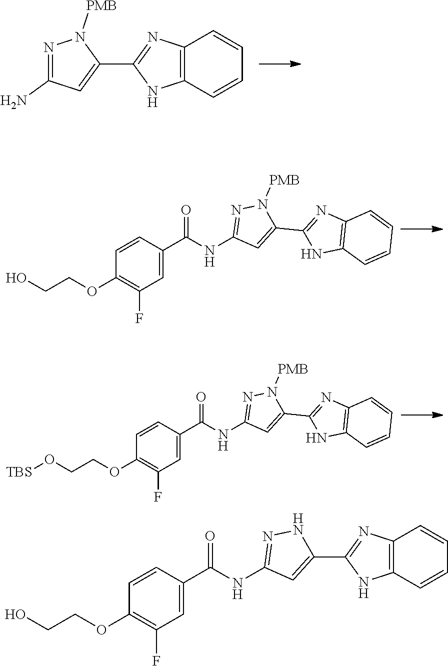

### Preparation of Compound A-130.

### Step 1: N-[5-(1H-benzimidazol-2-yl)-1-[(4-methoxyphenyl)methyl]pyrazol-3-yl]-3-fluoro-4-(2-hydroxyethoxy)benzamide

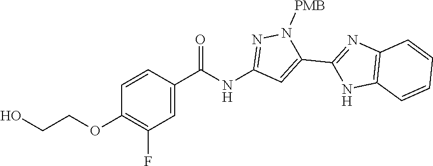

DIEA (0.29 μL, 1.64 mmol) was added to a solution of HATU (23.20 mg, 0.61 mmol) and 3-fluoro-4-(2-hydroxyethoxy)benzoic acid (K-11) (118 mg, 0.59 mmol) in DMF (2 mL) at 0° C. The mixture was stirred at 0° C. for 10 min. 5-(1H-Benzimidazol-2-yl)-1-[(4-methoxyphenyl)methyl]pyrazol-3-amine (J-1) (150 mg, 0.47 mmol) was added. The mixture was warmed to 23° C. and stirred for 24 h. Water (25 mL) was added, and the mixture was stirred at 23° C. for 5 min. The mixture was diluted with EtOAc, and the aq. phase was extracted with EtOAc. The combined organic phases were dried (MgSO₄), filtered, and concentrated to provide the impure title compound as a solid (236 mg). ¹H NMR (500 MHz, DMSO) δ 13.13 (s, 1H), 11.03 (s, 1H), 7.98-7.88 (m, 2H), 7.72 (d, J=7.8 Hz, 1H), 7.57-7.51 (m, 2H), 7.32-7.21 (m, 5H), 6.85 (d, J=8.7 Hz, 2H), 6.03 (s, 2H), 4.94 (t, J=5.4 Hz, 1H), 4.16 (t, J=4.8 Hz, 2H), 3.80-3.71 (m, 2H), 3.68 (s, 3H). m/z (ES⁺), [M+H]⁺ 502.0. HPLC (A05) t_{R}=2.38 min.

### Step 2: N-[5-(1H-benzimidazol-2-yl)-1-[(4-methoxyphenyl)methyl]pyrazol-3-yl]-4-[2-[tert-butyl(dimethyl)silyl]oxyethoxy]-3-fluoro-benzamide

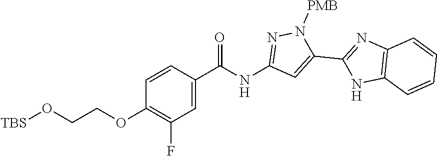

TBSOTf (0.33 mL, 1.41 mmol) was added to a solution of N-[5-(1H-benzimidazol-2-yl)-1-[(4 methoxyphenyl)methyl]pyrazol-3-yl]-3-fluoro-4-(2-hydroxyethoxy)benzamide (236 mg, 0.47 mmol) and NEt₃ (0.20 mL, 1.41 mmol) in THF (1.20 mL) at 0° C. The mixture was stirred at 23° C. for 2 h and diluted with sat. NaHCO₃ (7 mL). The aq. phase was extracted with EtOAc (3×10 mL). The combined organic phases were washed with brine (7 mL), dried (MgSO₄), filtered, and concentrated. The residue was purified by silica gel chromatography (25 g cartridge) eluting with hexane and EtOAc (0-100%) to provide the title compound as a solid (146 mg, 50%). ¹H NMR (400 MHz, DMSO) δ 13.13 (s, 1H), 11.05 (s, 1H), 8.02-7.85 (m, 2H), 7.77-7.65 (m, 1H), 7.63-7.43 (m, 2H), 7.40-7.00 (m, 5H), 6.99-6.75 (m, 2H), 6.14-5.91 (m, 2H), 4.31-4.09 (m, 2H), 4.08-3.81 (m, 2H), 3.81-3.57 (m, 3H), 0.85 (s, 9H), 0.06 (s, 6H). m/z (ES⁺), [M+H]⁺ 616.2. HPLC (A05) t_{R}=3.02 min.

### Step 3: N-[5-(1H-benzimidazol-2-yl)-1H-pyrazol-3-yl]-3-fluoro-4-(2-hydroxyethoxy)benzamide

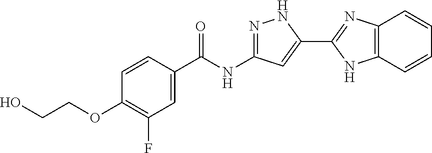 [SMILES: O=C(Nc1cc(-c2nc3ccccc3[nH]2)[nH]n1)c1ccc(OCCO)c(F)c1]

TFA (1 mL) was added to N-[5-(1H-benzimidazol-2-yl)-1-[(4-methoxyphenyl)methyl]pyrazol-3-yl]-4-[2-[tert-butyl(dimethyl)silyl]oxyethoxy]-3-fluoro-benzamide (146 mg, 0.237 mmol), and the mixture was stirred at 23° C. for 40 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with acetone (10 mL), DCM (10 mL), and acetone (10 mL) and dried to provide the title compound as a solid in a 2:1 mixture of pyrazole tautomers (80.2 mg, 89%). ¹H NMR (500 MHz, DMSO) δ 13.50 [13.15] (s, 1H), 13.04 [12.74] (s, 1H), 10.95 [11.13] (s, 1H), 2.03-7.78 (m, 2H), 7.72-7.60 (m, 1H), 7.57-7.46 (m, 1H), 7.43 [6.69] (s, 1H), 7.37-7.29 (m, 1H), 7.29-7.14 (m, 2H), 5.01-4.88 (m, 1H), 4.23-4.11 (m, 2H), 3.83-3.73 (m, 2H). ¹⁹F NMR (471 MHz, DMSO) δ −134.28 [−133.92]. m/z (ES⁺), [M+H]⁺ 382.5. HPLC (A05) t_{R}=1.96 min.

### Preparation of Compound A-141.

### Step 1: 3-chloro-N-[5-(5-fluoro-1H-benzimidazol-2-yl)-1-[(4-methoxyphenyl)-methyl]-pyrazol-3-yl]-4-(2-hydroxyethoxy)benzamide

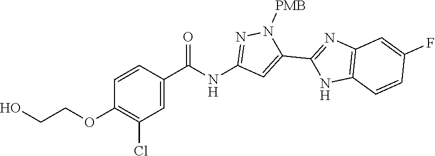

3-Chloro-4-(2-hydroxyethoxy)benzoic acid (K-8) (49.8 mg, 0.230 mmol) and HATU (0.0875 g, 0.230 mmol) were dissolved in DMF (1.50 mL) at 0° C. DIEA (0.0787 mL, 0.460 mmol) was added, and the mixture was stirred at 0° C. for 5 min. 5-(5-Fluoro-1H-benzimidazol-2-yl)-1-[(4-methoxyphenyl)methyl]pyrazol-3-amine (J-2) (77.6 mg, 0.230 mmol) was added, and the mixture was stirred at 23° C. for 18 h. EtOAc (100 mL) was added, and the organic phase was washed with water (3×50 mL) and brine (60 mL), dried (Na₂SO₄), filtered, and concentrated. The residue was purified by silica gel chromatography (12 g cartridge) eluting with hexanes and EtOAc (0-100%) to provide the title compound as a solid (69 mg, 56%). ¹H NMR (400 MHz, DMSO) δ 13.27 (s, 1H), 11.08 (s, 1H), 8.15 (d, J=2.3 Hz, 1H), 8.03 (dd, J=8.7, 2.3 Hz, 1H), 7.57-7.50 (m, 2H), 7.39-7.30 (m, 1H), 7.27 (d, J=8.8 Hz, 1H), 7.23 (d, J=8.7 Hz, 2H), 7.18-7.06 (m, 1H), 6.88-6.83 (m, 2H), 6.01 (s, 2H), 4.93 (s, 1H), 4.18 (t, J=4.9 Hz, 2H), 3.76 (t, J=5.4 Hz, 2H), 3.69 (s, 3H). m/z (ES⁺), [M+H]⁺ 536.4. HPLC (A05) t_{R}=2.47 min.

### Step 2: 4-[2-[tert-butyl(dimethyl)silyl]oxyethoxy]-3-chloro-N-[5-(5-fluoro-1H-benzimidazol-2-yl)-1-[(4-methoxyphenyl)methyl]pyrazol-3-yl]benzamide

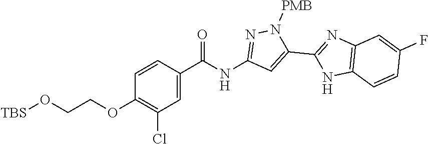

TBSOTf (0.103 mL, 0.448 mmol) was added to a solution of 3-chloro-N-[5-(5-fluoro-1H-benzimidazol-2-yl)-1-[(4-methoxyphenyl)methyl]pyrazol-3-yl]-4-(2-hydroxyethoxy)benzamide (80 mg, 0.149 mmol) and NEt₃ (0 mL, 0.448 mmol) in THF (1 mL) at 0° C. The mixture was stirred at 0° C. for 2 h and diluted with sat. NaHCO₃ (25 mL). The aqueous phase was extracted with EtOAc (3×50 mL). The combined organic phases were washed with brine (30 mL), dried (Na₂SO₄), filtered, and concentrated. The residue was purified by silica gel chromatography (12 g cartridge) with hexane and EtOAc (0-100%) to provide the title compound as a solid (50.4 mg, 52%). ¹H NMR (400 MHz, DMSO) δ 13.28 (s, 1H), 11.08 (s, 1H), 8.15 (d, J=2.3 Hz, 1H), 8.03 (dd, J=8.7, 2.4 Hz, 1H), 7.72 [7.36] (s, 1H), 7.57-7.50 (m, 2H), 7.27 (d, J=8.9 Hz, 1H), 7.23 (d, J=8.8 Hz, 2H), 7.15-7.04 (m, 1H), 6.88-6.83 (m, 2H), 6.01 (s, 2H), 4.24-4.20 (m, 2H), 3.99-3.94 (m, 2H), 3.69 (s, 3H), 0.87 (s, 9H), 0.08 (s, 6H). m/z (ES⁺), [M+H]⁺ 651.3. HPLC (A05) t_{R}=3.09 min.

### Step 3: 3-chloro-N-[5-(5-fluoro-1H-benzimidazol-2-yl)-1H-pyrazol-3-yl]-4-(2-hydroxyethoxy)benzamide

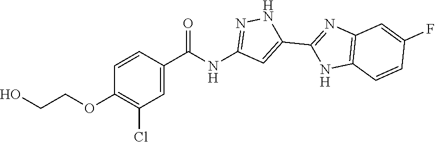 [SMILES: O=C(Nc1cc(-c2nc3cc(F)ccc3[nH]2)[nH]n1)c1ccc(OCCO)c(Cl)c1]

TFA (1 mL) was added to 4-[2-[tert-butyl(dimethyl)silyl]oxyethoxy]-3-chloro-N-[5-(5-fluoro-1H-benzimidazol-2-yl)-1-[(4-methoxyphenyl)methyl]pyrazol-3-yl]benzamide (50.4 mg, 0.0775 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The solid was purified by preparative HPLC (BEH C18 30×100) eluting with water (10 mM (NH₄)(HCO₂)) and MeCN (35-45%) to provide the title compound as a solid in a 4:1 mixture of tautomers (10.3 mg, 32%). ¹H NMR (400 MHz, DMSO) δ 11.04 (s, 1H), 8.33 (s, 1H), 8.15 (d, J=2.2 Hz, 1H), 8.03 (dd, J=8.7, 2.2 Hz, 1H), 7.60 (dd, J=47.4, 14.9 Hz, 1H), 7.45-7.35 (m, 1H), 7.31 (d, J=9.1 Hz, 1H), 7.07 (t, J=8.6 Hz, 1H), 4.94 (s, 1H), 4.19 (t, J=5.0 Hz, 2H), 3.78 (t, J=4.5 Hz, 2H). m/z (ES⁺), [M+H]⁺ 416.5. HPLC (A05) t_{R}=2.09 min.

### EXAMPLE B27

### Preparation of Compounds A-138 and A-140

### Representative Scheme for Synthesis of Compounds A-138 and A-140.

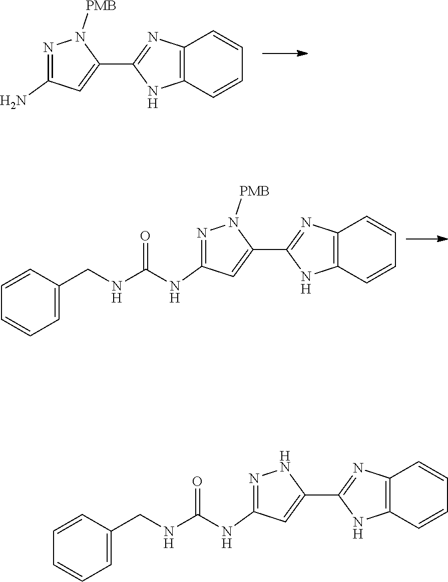

### Preparation of Compound A-138.

### Step 1: 1-[5-(1H-benzimidazol-2-yl)-1-[(4-methoxyphenyl)methyl]pyrazol-3-yl]-3-benzyl-urea

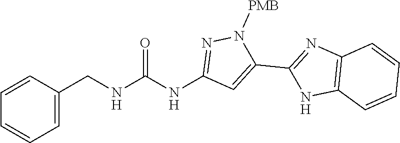

Benzylisocyanate (44.5 μL, 0.360 mmol) was dissolved in DMF (3 mL), and the mixture was cooled to 0° C. NEt₃ (62.7 μL, 0.450 mmol) was added, and the mixture was stirred at 0° C. for 10 min. 5-(1H-Benzimidazol-2-yl)-1-[(4-methoxyphenyl)methyl]pyrazol-3-amine (J-1) (95.8 mg, 0.300 mmol) was added. The mixture was warmed to 23° C. and stirred for 18 h. Water (50 mL) was added. The aq. phase was extracted with EtOAc (3×50 mL). The combined organic phases were washed with brine (50 mL), dried (MgSO₄), filtered, and concentrated. The residue was purified by silica gel chromatography (25 g cartridge) eluting with DCM and MeOH (0-10%) to provide the title compound as a solid (44.9 mg, 33%). ¹H NMR (400 MHz, DMSO) δ 13.04 (s, 1H), 9.11 (s, 1H), 7.77-7.63 (m, 1H), 7.58-7.46 (m, 1H), 7.39-7.17 (m, 9H), 7.08 (s, 1H), 7.00-6.90 (m, 1H), 6.86-6.79 (m, 2H), 5.92 (s, 2H), 4.35 (d, J=5.9 Hz, 2H), 3.69 (s, 3H). m/z (ES⁺), [M+H]⁺ 453.6. HPLC (A05) t_{R}=2.50 min.

### Step 2: 1-[5-(1H-benzimidazol-2-yl)-1H-pyrazol-3-yl]-3-benzyl-urea

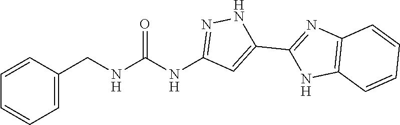 [SMILES: O=C(NCc1ccccc1)Nc1cc(-c2nc3ccccc3[nH]2)[nH]n1]

TFA (1 mL) was added to 1-[5-(1H-benzimidazol-2-yl)-1-[(4-methoxyphenyl)methyl]pyrazol-3-yl]-3-benzyl-urea (42.8 mg, 0.0946 mmol), and the mixture was stirred at 23° C. for 45 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL) and dried under high vacuum to provide the title compound as a solid in a 3:2 mixture of pyrazole tautomers (11.6 mg, 37%). ¹H NMR (400 MHz, DMSO) δ 13.15 (s, 1H), 12.98 [12.73] (s, 1H), 9.02 [9.23] (s, 1H), 7.81-7.59 (m, 1H), 7.59-7.43 (m, 1H), 7.41-7.30 (m, 4H), 7.30-7.06 (m, 4H), 7.00-6.87 (m, 1H), 4.38 [4.36] (s, 2H). m/z (ES⁺), [M+H]⁺ 333.6. HPLC (A05) t_{R}=2.07 min.

### Preparation of Compound A-140.

### Step 1: 1-[5-(1H-benzimidazol-2-yl)-1-[(4-methoxyphenyl)methyl]pyrazol-3-yl]-3-phenyl-urea

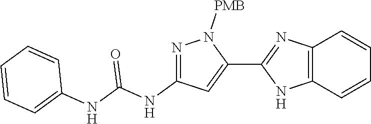

Phenylisocyanate (39.3 μL, 0.360 mmol) was dissolved in DMF (3 mL), and the mixture was cooled to 0° C. NEt₃ (62.7 μL, 0.450 mmol) was added, and the mixture was stirred at 0° C. for 10 min. 5-(1H-Benzimidazol-2-yl)-1-[(4-methoxyphenyl)methyl]pyrazol-3-amine (J-1) (95.8 mg, 0.300 mmol) was added. The mixture was warmed to 23° C. and stirred for 18 h. Water (50 mL) was added. The aq. phase was extracted with EtOAc (3×50 mL). The combined organic phases were washed with brine (50 mL), dried (MgSO₄), filtered, and concentrated. The residue was purified by silica gel chromatography (25 g cartridge) eluting with DCM and MeOH (0-10%) to provide the title compound as a solid (59.9 mg, 46%). ¹H NMR (400 MHz, DMSO) δ 13.09 (s, 1H), 9.21 (s, 1H), 8.85 (s, 1H), 7.79-7.65 (m, 1H), 7.60-7.50 (m, 1H), 7.49-7.43 (m, 2H), 7.36-7.16 (m, 7H), 7.04-6.96 (m, 1H), 6.91-6.82 (m, 2H), 5.98 (s, 2H), 3.70 (s, 3H). m/z (ES⁺), [M+H]⁺ 439.6. HPLC (A05) t_{R}=2.54 min.

### Step 2: 1-[5-(1H-benzimidazol-2-yl)-1H-pyrazol-3-yl]-3-phenyl-urea

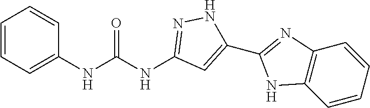 [SMILES: O=C(Nc1ccccc1)Nc1cc(-c2nc3ccccc3[nH]2)[nH]n1]

TFA (1 mL) was added to 1-[5-(1H-benzimidazol-2-yl)-1-[(4-methoxyphenyl)methyl]pyrazol-3-yl]-3-phenyl-urea (57 mg, 0.130 mmol), and the mixture was stirred at 23° C. for 45 min. The mixture was concentrated. The residue was dissolved in DMSO (0.500 mL). NEt₃ (0.500 mL) was added, and the mixture was stirred at 23° C. for 5 min. Water (10 mL) was added. The mixture was filtered, and the solid was washed with water (10 mL), acetone (10 mL), DCM (10 mL), and acetone (10 mL). The solid was purified by preparative HPLC (BEH C18 30×150) eluting with water (10 mM (NH₄)(HCO₃)) and MeCN (37-47%) to provide the title compound as a solid (3.6 mg, 9%). ¹H NMR (500 MHz, DMSO) δ 13.08 (s, 2H), 9.10 (s, 2H), 7.65-7.51 (m, 2H), 7.53-7.47 (m, 2H), 7.37-7.25 (m, 2H), 7.27-7.17 (m, 2H), 7.09-6.92 (m, 2H). m/z (ES⁺), [M+H]⁺ 319.5. HPLC (A05) t_{R}=2.09 min.

### EXAMPLE B28

### Preparation of Compounds A-145, A-146, A-147, A-151, and A-155

### Representative Synthetic Scheme for the Syntheses of Compounds A-145, A-146, A-147, A-151, and A-155.

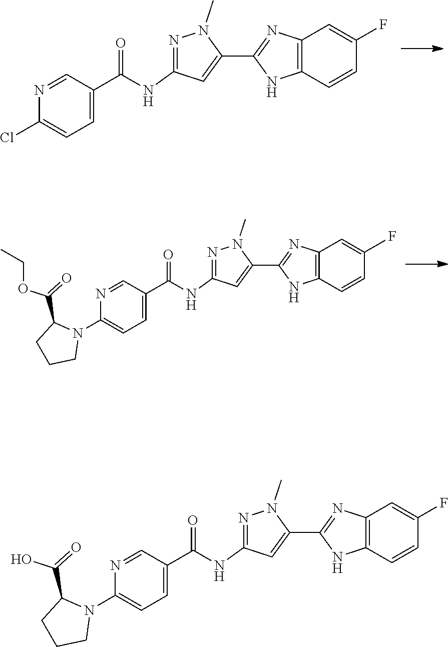 [SMILES: Cn1nc(NC(=O)c2ccc(Cl)nc2)cc1-c1nc2cc(F)ccc2[nH]1 ; CCOC(=O)[C@@H]1CCCN1c1ccc(C(=O)Nc2cc(-c3nc4cc(F)ccc4[nH]3)n(C)n2)cn1 ; Cn1nc(NC(=O)c2ccc(N3CCC[C@H]3C(=O)O)nc2)cc1-c1nc2cc(F)ccc2[nH]1]

### Preparation of Compound A-145.

### Step 1: ethyl (25)-1-[5-[[5-(5-fluoro-1H-benzimidazol-2-yl)-1-methyl-pyrazol-3-yl]carbamoyl]-2-pyridyl]pyrrolidine-2-carboxylate

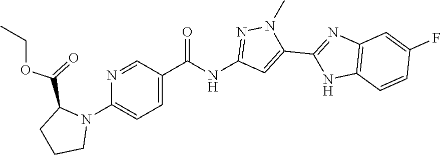 [SMILES: CCOC(=O)[C@@H]1CCCN1c1ccc(C(=O)Nc2cc(-c3nc4cc(F)ccc4[nH]3)n(C)n2)cn1]

Ethyl (2S)-pyrrolidine-2-carboxylate (29 mg, 0.202 mmol) was dissolved in EtOH (1 mL). 6-Chloro-N-[5-(5-fluoro-1H-benzimidazol-2-yl)-1-methyl-pyrazol-3-yl]pyridine-3-carboxamide (L-12) (50 mg, 0.135 mmol) and DIEA (47.0 μL, 0.270 mmol) were added. The mixture was heated at 100° C. for 5 days. The mixture was concentrated. Sat. NaHCO₃ (20 mL) was added, and the aq. phase was extracted with EtOAc (3×20 mL). The combined organic phases were washed with sat. NaHCO₃ (20 mL) and brine (20 mL), dried (MgSO₄), filtered, and concentrated. The residue was purified by silica gel chromatography (12 g cartridge) eluting with hexanes and EtOAc (0-100%) and by preparative HPLC (BEH C18 30×150) eluting with water (10 mM (NH₄)(HCO₃)) and MeCN (45-55%) to provide the title compound as a solid (16.1 mg, 25%). ¹H NMR (500 MHz, DMSO) δ 12.90 (s, 1H), 10.82 (s, 1H), 8.74 (d, J=2.4 Hz, 1H), 8.17 (dd, J=8.9, 2.5 Hz, 1H), 7.63 (s, 1H), 7.47 (s, 1H), 7.46-7.33 (m, 1H), 7.11 (t, J=9.2 Hz, 1H), 6.58 (d, J=8.8 Hz, 1H), 4.59 (dd, J=8.5, 2.6 Hz, 1H), 4.29 (s, 3H), 4.12 (qd, J=7.0, 1.8 Hz, 2H), 3.66-3.55 (m, 1H), 3.55-3.46 (m, 1H), 2.42-2.22 (m, 1H), 2.17-1.93 (m, 3H), 1.19 (t, J=7.1 Hz, 3H). ¹⁹F NMR (471 MHz, DMSO) δ −118.77 [−121.25]. m/z (ES⁺), [M+H]⁺ 478.6. HPLC (A05) t_{R}=2.35 min.

### Preparation of Compound A-151 ((2S)-1-[5-[[5-(5-fluoro-1H-benzimidazol-2-yl)-1-methyl-pyrazol-3-yl]carbamoyl]-2-pyridyl]pyrrolidine-2-carboxylic acid)

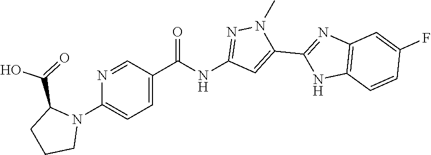 [SMILES: Cn1nc(NC(=O)c2ccc(N3CCC[C@H]3C(=O)O)nc2)cc1-c1nc2cc(F)ccc2[nH]1]

LiOH—H₂O (7 mg, 0.168 mmol) was added to a mixture of ethyl (2S)-1-[5-[[5-(5-fluoro-1H-benzimidazol-2-yl)-1-methyl-pyrazol-3-yl]carbamoyl]-2-pyridyl]pyrrolidine-2-carboxylate (A-145) (9.2 mg, 0.0193 mmol) in THF (0.400 mL) and water (0.400 mL). The mixture was stirred at 23° C. for 1.5 h and concentrated. Water (10 mL) was added, and the aq. phase was acidified to pH 3 with 1M HCl (200 μL). The aq. phase was extracted with EtOAc (2×10 mL). The combined organic phases were concentrated to provide the title compound as a solid (6.4 mg, 74%). ¹H NMR (400 MHz, CD₃OD) δ 8.70 (d, J=2.4 Hz, 1H), 8.09 (dd, J=8.9, 2.4 Hz, 1H), 7.71-7.53 (m, 1H), 7.43-7.28 (m, 1H), 7.26 (s, 1H), 7.08 (td, J=9.5, 1.8 Hz, 1H), 6.59 (d, J=9.0 Hz, 1H), 4.65-4.53 (m, 1H), 4.25 (s, 3H), 3.90-3.63 (m, 1H), 3.62-3.53 (m, 1H), 2.44-2.28 (m, 1H), 2.27-2.04 (m, 3H). m/z (ES⁺), [M+H]⁺ 450.5. HPLC (A05) t_{R}=1.96 min.

### Preparation of Compound A-146 (ethyl (2R)-1-[5-[[5-(5-fluoro-1H-benzimidazol-2-yl)-1-methyl-pyrazol-3-yl]carbamoyl]-2-pyridyl]pyrrolidine-2-carboxylate)

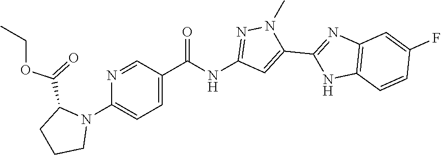 [SMILES: CCOC(=O)[C@H]1CCCN1c1ccc(C(=O)Nc2cc(-c3nc4cc(F)ccc4[nH]3)n(C)n2)cn1]

Ethyl (2R)-pyrrolidine-2-carboxylate hydrochloride (36.3 mg, 0.202 mmol) was dissolved in EtOH (1 mL). 6-Chloro-N-[5-(5-fluoro-1H-benzimidazol-2-yl)-1-methyl-pyrazol-3-yl]-pyridine-3-carboxamide (L-12) (50 mg, 0.135 mmol) and DIEA (70.5 μL, 0.405 mmol) were added. The mixture was heated at 100° C. for 5 days. The mixture was concentrated. Sat. NaHCO₃ (20 mL) was added, and the aq. phase was extracted with EtOAc (3×20 mL). The combined organic phases were washed with sat. NaHCO₃ (20 mL) and brine (20 mL), dried (MgSO₄), filtered, and concentrated. The residue was purified by silica gel chromatography (12 g cartridge) eluting with hexanes and EtOAc (0-100%) and by preparative HPLC (BEH C18 30×150) eluting with water (10 mM (NH₄)(HCO₃)) and MeCN (45-55%). The HPLC solution was filtered, and the filtrate was acidified to pH 3 with conc. HCl. The mixture was concentrated. Water (20 mL) was added to the residue, and the aq. phase was extracted with EtOAc (3×20 mL). The combined organic phases were dried (MgSO₄), filtered, and concentrated to provide the title compound as a solid (7.3 mg, 11%). ¹EINMR (500 MHz, DMSO) δ 13.13 (s, 1H), 10.81 (s, 1H), 8.73 (d, J=2.5 Hz, 1H), 8.17 (dd, J=8.9, 2.5 Hz, 1H), 7.62 (s, 1H), 7.46 (s, 1H), 7.41 (s, 1H), 7.10 (t, J=9.3 Hz, 1H), 6.58 (d, J=8.9 Hz, 1H), 4.59 (dd, J=8.2, 2.4 Hz, 1H), 4.29 (s, 3H), 4.11 (qd, J=7.1, 1.9 Hz, 2H), 3.63-3.56 (m, 1H), 3.56-3.44 (m, 1H), 2.40-2.24 (m, 1H), 2.21-1.92 (m, 3H), 1.19 (t, J=7.0 Hz, 3H). ¹⁹F NMR (471 MHz, DMSO) δ −118.77 [−121.25]. m/z (ES⁺), [M+H]⁺ 478.6. HPLC (A05) t_{R}=2.35 min.

### Preparation of Compound A-147 (N-[5-(1H-benzimidazol-2-yl)-1-[(4-methoxyphenyl)methyl]pyrazol-3-yl]-4-(2-hydroxyethoxy)-3-methoxy-benzamide)

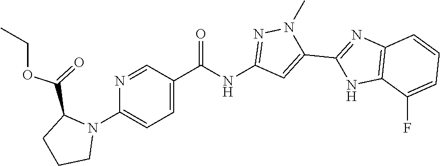 [SMILES: CCOC(=O)[C@@H]1CCCN1c1ccc(C(=O)Nc2cc(-c3nc4cccc(F)c4[nH]3)n(C)n2)cn1]

6-Chloro-N-[5-(4-fluoro-1H-benzimidazol-2-yl)-1-methyl-pyrazol-3-yl]pyridine-3-carboxamide (L-13) (60 mg, 0.162 mmol), ethyl (2S)-pyrrolidine-2-carboxylate (0.0927 g, 0.647 mmol), and DIEA (0.111 mL, 0.647 mmol) were dissolved in EtOH (1.50 mL). The mixture was stirred at 100° C. for 72 h. The mixture was concentrated. The residue was purified by silica gel chromatography (12 g cartridge) eluting with hexanes and EtOAc (0-100%) and by preparative HPLC eluting with water (10 mM (NH₄)(HCO₃)) and MeCN (51-61%) to provide the title compound as a solid (22.5 mg, 29%). ¹H NMR (400 MHz, DMSO) δ 13.37 (s, 1H), 10.83 (s, 1H), 8.73 (d, J=2.2 Hz, 1H), 8.16 (dd, J=8.9, 2.4 Hz, 1H), 7.49 (s, 1H), 7.37 (d, J=8.3 Hz, 1H), 7.23 (dd, J=12.7, 7.8 Hz, 1H), 7.10-7.00 (m, 1H), 6.58 (d, J=8.8 Hz, 1H), 4.58 (dd, J=8.5, 2.5 Hz, 1H), 4.30 (s, 3H), 4.11 (qd, J=7.0, 1.1 Hz, 2H), 3.62-3.48 (m, 2H), 2.37-2.29 (m, 1H), 2.03 (t, J=9.9 Hz, 3H), 1.18 (t, J=7.1 Hz, 3H). ¹⁹F NMR (376 MHz, DMSO) δ −128.76 (s). m/z (ES⁺), [M+H]⁺ 478.3. HPLC (A05) t_{R}=2.37 min.

### Preparation of Compound A-155 ((2S)-1-[5-[[5-(7-fluoro-1H-benzimidazol-2-yl)-1-methyl-pyrazol-3-yl]carbamoyl]-2-pyridyl]pyrrolidine-2-carboxylic acid)

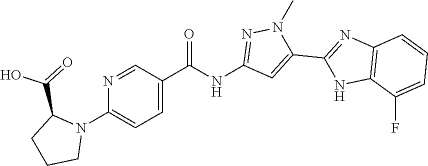 [SMILES: Cn1nc(NC(=O)c2ccc(N3CCC[C@H]3C(=O)O)nc2)cc1-c1nc2cccc(F)c2[nH]1]

LiOH—H₂O (0.0119 g, 0.283 mmol) was added to a solution of ethyl (2S)-1-[5-[[5-(4-fluoro-1H-benzimidazol-2-yl)-1-methyl-pyrazol-3-yl]carbamoyl]-2-pyridyl]pyrrolidine-2-carboxylate (22.5 mg, 0.0471 mmol) in MeOH (0.500 mL), THF (0.500 mL), and water (0.500 mL). The solution was stirred at 23° C. for 1 h. The mixture was concentrated, and water (5 mL) was added. The aq. phase was washed with EtOAc (3×20 mL) and acidified to pH 3 with 1M HCl. The aq. phase was extracted with EtOAc (3×20 mL), and the combined organic phase was concentrated to provide the title compound as a solid (10.9 mg, 52%). ¹H NMR (400 MHz, DMSO) δ 10.80 (s, 1H), 8.74 (d, J=2.2 Hz, 1H), 8.12 (d, J=8.6 Hz, 1H), 7.52 (s, 1H), 7.39 (d, J=7.8 Hz, 1H), 7.24 (td, J=7.9, 4.8 Hz, 1H), 7.06 (dd, J=10.8, 8.1 Hz, 1H), 6.50 (s, 1H), 4.54-4.36 (m, 1H), 4.30 (s, 3H), 3.54 (d, J=19.1 Hz, 2H), 2.23 (dd, J=17.5, 9.6 Hz, 1H), 2.08-1.95 (m, 3H). m/z (ES⁺), [M−H]⁻ 448.6. HPLC (A05) t_{R}=1.97 min.

### EXAMPLE B29

### Preparation of Compounds A-174 and A-181

### Representative Scheme for Synthesis of Compounds A-1 74 and A-181.

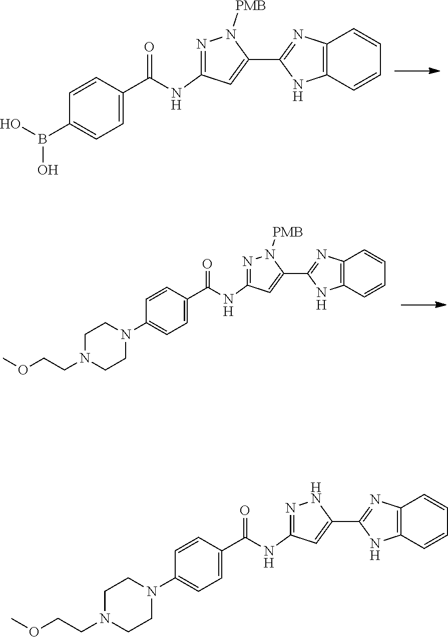

### Preparation of Compound A-174.

### Step 1: N-[5-(1H-benzimidazol-2-yl)-1-[(4-methoxyphenyl)methyl]pyrazol-3-yl]-4-[4-(2-methoxyethyl)piperazin-1-yl]benzamide

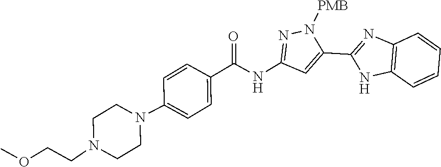

DMAP (0.0235 g, 0.193 mmol) was added to a solution of [4-[[5-(1H-benzimidazol-2-yl)-1-[(4-methoxyphenyl)methyl]pyrazol-3-yl]carbamoyl]phenyl]boronic acid (L-19) (60 mg, 0.128 mmol), 1-(2-methoxyethyl)piperazine (0.0382 mL, 0.257 mmol), and Cu(OAc)₂ (0.0280 g, 0.154 mmol) in DMSO (1 mL) and DCE (1 mL). The mixture was stirred at 23° C. for 18 h and at 50° C. for 24 h. The mixture was filtered, and the filtrate was diluted with EtOAc (100 mL). The organic phase was washed 5% aq. NH₄OH (50 mL), water (2×50 mL), and brine (50 mL). The organic phase was dried (Na₂SO₄), filtered, and concentrated. The residue was purified by silica gel chromatography (12 g cartridge) eluting with DCM and MeOH (0-10%) to provide the title compound as a solid (12 mg, 17%). ¹H NMR (400 MHz, DMSO) δ 13.11 (s, 1H), 10.75 (s, 1H), 7.95 (d, J=9.1 Hz, 2H), 7.71 (d, J=7.7 Hz, 1H), 7.54 (d, J=8.8 Hz, 2H), 7.30-7.20 (m, 4H), 6.96 (d, J=9.1 Hz, 2H), 6.88-6.82 (m, 2H), 6.02 (s, 2H), 3.68 (s, 3H), 3.47 (t, J=5.8 Hz, 2H), 3.30-3.21 (m, 7H), 2.57-2.52 (m, 6H). m/z (ES⁺), [M+H]⁺ 566.3. HPLC (A05) t_{R}=2.17 min.

### Step 2: N-[5-(1H-benzimidazol-2-yl)-1H-pyrazol-3-yl]-4-[4-(2-methoxyethyl)-piperazin-1-yl]benzamide

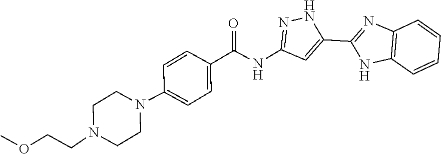 [SMILES: COCCN1CCN(c2ccc(C(=O)Nc3cc(-c4nc5ccccc5[nH]4)[nH]n3)cc2)CC1]

TFA (1 mL) was added to N-[5-(1H-benzimidazol-2-yl)-1-[(4-methoxyphenyl)methyl]pyrazol-3-yl]-4-[4-(2-methoxyethyl)piperazin-1-yl]benzamide (20 mg, 0.0354 mmol), and the mixture was stirred at 23° C. for 1 h. The mixture was concentrated. The residue was purified by preparative HPLC (BEH C18 30×150) eluting with water (10 mM (NH₄)(HCO₃)) and MeCN (30-40%) to provide the title compound as a solid (1.70 mg, 9%). ¹H NMR (400 MHz, DMSO) δ 7.94 (d, J=8.9 Hz, 2H), 7.59-7.52 (m, 2H), 7.20-7.15 (m, 2H), 7.13-7.06 (m, 1H), 7.00 (d, J=9.1 Hz, 2H), 3.48 (t, J=5.7 Hz, 2H), 3.25 (s, 3H), 2.59-2.52 (m, 8H), 1.98 (d, J=7.5 Hz, 2H). m/z (ES⁺), [M+H]⁺ 446.7. HPLC (A05) t_{R}=1.81 min.

### Preparation of Compound A-181.

### Step 1: N-[5-(1H-benzimidazol-2-yl)-1-[(4-methoxyphenyl)methyl]pyrazol-3-yl]-4-[4-(2-hydroxyethyl)piperazin-1-yl]benzamide

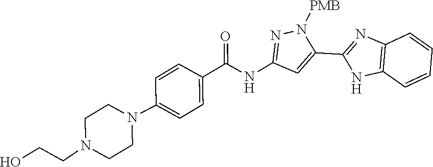

DMAP (0.0784 g, 0.642 mmol) was added to a solution of [4-[[5-(1H-benzimidazol-2-yl)-1-[(4-methoxyphenyl)methyl]pyrazol-3-yl]carbamoyl]phenyl]boronic acid (L-19) (150 mg, 0.321 mmol), 2-piperazin-1-ylethanol (0.0955 mL, 0.642 mmol), and Cu(OAc)₂ (0.0875 g, 0.482 mmol) in DMSO (5 mL). The mixture was heated at 50° C. for 18 h. EtOAc (100 mL) was added, and the organic phase was washed with 5% aq. NH₄OH (50 mL), water (2×50 mL), and brine (50 mL). The organic phase was dried (Na₂SO₄), filtered, and concentrated. The residue was purified by silica gel chromatography (12 g cartridge) eluting with DCM and MeOH (0-10%) to provide the title compound as a solid (40 mg, 23%). ¹H NMR (500 MHz, DMSO) δ 13.10 (s, 1H), 10.74 (s, 1H), 7.95 (d, J=9.1 Hz, 2H), 7.71 (d, J=8.0 Hz, 1H), 7.56-7.51 (m, 2H), 7.30-7.20 (m, 4H), 6.96 (d, J=9.0 Hz, 2H), 6.87-6.83 (m, 2H), 6.02 (s, 2H), 4.43 (t, J=5.2 Hz, 1H), 3.68 (s, 3H), 3.54 (dd, J=11.6, 6.0 Hz, 2H), 3.29-3.26 (m, 4H), 2.57-2.53 (m, 4H), 2.44 (t, J=5.8 Hz, 2H). m/z (ES⁺), [M+H]⁺ 552.8. HPLC (A05) t_{R}=2.08 min.

### Step 2: N-[5-(1H-benzimidazol-2-yl)-1H-pyrazol-3-yl]-4-[4-(2-methoxyethyl)-piperazin-1-yl]benzamide

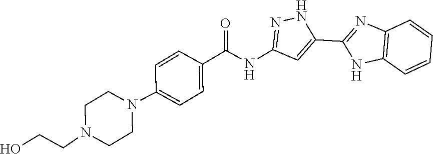 [SMILES: O=C(Nc1cc(-c2nc3ccccc3[nH]2)[nH]n1)c1ccc(N2CCN(CCO)CC2)cc1]

TFA (1 mL) was added to N-[5-(1H-benzimidazol-2-yl)-1-[(4-methoxyphenyl)methyl]pyrazol-3-yl]-4-[4-(2-hydroxyethyl)piperazin-1-yl]benzamide (40 mg, 0.0725 mmol), and the mixture was stirred at 23° C. for 1 h. The mixture was concentrated. The residue was purified by preparative HPLC (BEH C18 30×150) eluting with water (10 mM (NH₄)(HCO₃)) and MeCN (21-41%) to provide the title compound as a solid (11.6 mg, 37%). ¹H NMR (400 MHz, DMSO) δ 13.21 (s, 1H), 12.85 (s, 1H), 10.66 (s, 1H), 7.90 (d, J=8.9 Hz, 2H), 7.62-7.42 (m, 2H), 7.16 (dt, J=7.6, 3.9 Hz, 2H), 6.97 (d, J=8.9 Hz, 2H), 4.56-4.23 (m, 1H), 3.51 (t, J=6.2 Hz, 2H), 3.28-3.24 (m, 4H), 2.51 (dd, J=8.5, 3.5 Hz, 4H), 2.40 (t, J=6.2 Hz, 2H). m/z (ES⁺), [M+H]⁺ 432.3. HPLC (A05) t_{R}=1.78 min.

### EXAMPLE B30

### Preparation of Compounds A-196, A-198, A-200, and A-201

### Representative Scheme for Synthesis of Compounds A-193, A-198, A-200, and A-201.

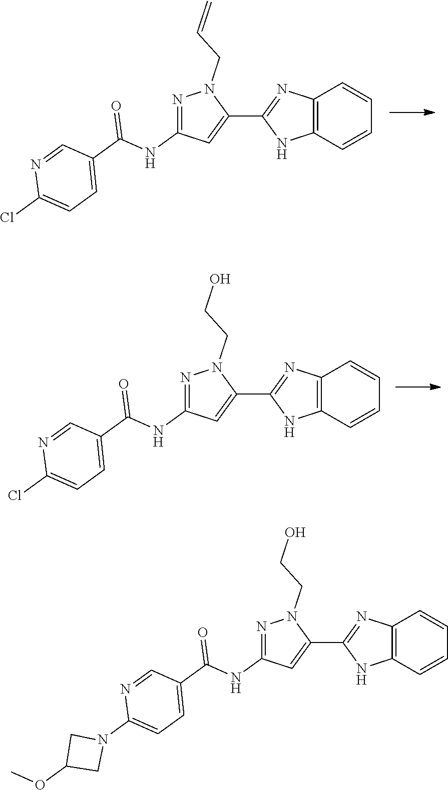 [SMILES: C=CCn1nc(NC(=O)c2ccc(Cl)nc2)cc1-c1nc2ccccc2[nH]1 ; O=C(Nc1cc(-c2nc3ccccc3[nH]2)n(CCO)n1)c1ccc(Cl)nc1 ; COC1CN(c2ccc(C(=O)Nc3cc(-c4nc5ccccc5[nH]4)n(CCO)n3)cn2)C1]

### Preparation of Compound A-196.

### Step 1: N-[5-(1H-benzimidazol-2-yl)-1-(2-hydroxyethyl)pyrazol-3-yl]-6-chloro-pyridine-3-carboxamide

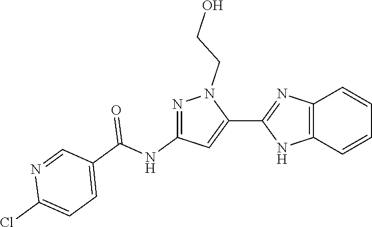 [SMILES: O=C(Nc1cc(-c2nc3ccccc3[nH]2)n(CCO)n1)c1ccc(Cl)nc1]

1,4-Dioxane (3 mL) was added to N-[1-allyl-5-(1H-benzimidazol-2-yl)pyrazol-3-yl]-3-chloro-4-methoxy-benzamide (105 mg, 0.277 mmol), and the mixture was cooled to 0° C. 2,6-Lutidine (0.06 mL, 0.554 mmol) and NaIO₄ (178 mg, 0.832 mmol) were added. A solution of potassium osmate(IV) dihydrate (5.11 mg, 0.0139 mmol) in water (1 mL) was added. The mixture was warmed to 23° C. and stirred for 18 h. Water (50 mL) was added, and the aq. phase was extracted with EtOAc (3×25 mL). The combined organic phases were washed with brine (40 mL), dried (MgSO₄), filtered, and concentrated. The residue (m/z (ES⁻), [M−H]⁻ 408.5. HPLC (A05) t_{R}=2.17 min) was suspended in MeOH (3 mL), and NaBH₄ (15.7 mg, 0.416 mmol) was added. The mixture was stirred at 40° C. for 1 h. The mixture was cooled to 23° C., and water (5 mL) was added. The aq. phase was extracted with EtOAc (3×5 mL). The combined organic phases were washed with brine (10,0 mL), dried (MgSO₄), filtered, and concentrated. The residue was purified by silica gel chromatography (12 g cartridge) eluting with hexanes and EtOAc (40-100%) to provide the title compound as a solid (17 mg, 16%). ¹H NMR (400 MHz, DMSO) δ 13.21-13.02 (m, 1H), 11.46 (s, 1H), 9.01 (d, J=2.3 Hz, 1H), 8.42 (dd, J=8.4, 2.5 Hz, 1H), 7.70 (d, J=8.1 Hz, 2H), 7.58-7.51 (m, 1H), 7.50 (s, 1H), 7.26 (s, 2H), 5.03-4.95 (m, 1H), 4.88 (t, J=6.2 Hz, 2H), 3.85 (s, 2H). m/z (ES⁺), [M−H]⁻ 381.6. HPLC (A05) t_{R}=2.06 min.

### Step 2: N-[5-(1H-benzimidazol-2-yl)-1-(2-hydroxyethyl)pyrazol-3-yl]-6-(3-methoxyazetidin-1-yl)pyridine-3-carboxamide

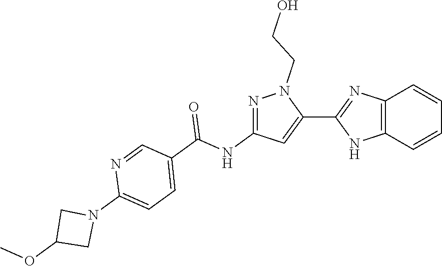 [SMILES: COC1CN(c2ccc(C(=O)Nc3cc(-c4nc5ccccc5[nH]4)n(CCO)n3)cn2)C1]

DIEA (0.0137 mL, 0.0784 mmol) was added to a solution of 3-methoxyazetidine hydrochloride (7.20 mg, 0.0588 mmol) and N-[5-(1H-benzimidazol-2-yl)-1-(2-hydroxyethyl)pyrazol-3-yl]-6-chloro-pyridine-3-carboxamide (L-21) (15 mg, 0.0392 mmol) in DMF (1 mL). The mixture was heated at 80° C. for 96 h. The mixture was cooled to 23° C., and water (10 mL) was added. The aq. phase was extracted with EtOAc (3×10 mL). The combined organic phases were washed with sat. NaHCO₃ (10 mL) and brine (10 mL), dried (MgSO₄), filtered, and concentrated. The residue was purified by preparative HPLC (BEH C18 30×150) eluting with water 10 mM (NH₄)(HCO₃)) and MeCN (25-35%) to provide the title compound as a solid (3.10 mg, 18%). ¹H NMR (400 MHz, DMSO) δ 13.15-12.90 (m, 1H), 10.87 (s, 1H), 8.78 (d, J=2.4 Hz, 1H), 8.14 (dd, J=8.8, 2.4 Hz, 1H), 7.75-7.49 (m, 3H), 7.44 (s, 1H), 7.25 (s, 2H), 6.43 (d, J=8.8 Hz, 1H), 4.85 (t, J=6.2 Hz, 2H), 4.36 (s, 1H), 4.26-4.21 (m, 2H), 3.85 (dd, J=9.9, 3.9 Hz, 4H), 3.27 (s, 3H). m/z (ES⁺), [M+H]⁺ 434.4. HPLC (A05) t_{R}=2.12 min.

### Preparation of Compound A-198 (N-[5-(1H-benzimidazol-2-yl)-1-(2-hydroxyethyl)pyrazol-3-yl]-6-[4-(2-methoxyethyl)piperazin-1-yl]pyridine-3-carboxamide)

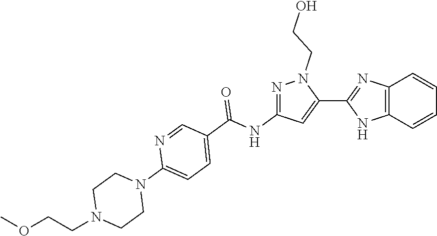 [SMILES: COCCN1CCN(c2ccc(C(=O)Nc3cc(-c4nc5ccccc5[nH]4)n(CCO)n3)cn2)CC1]

DIEA (0.00910 mL, 0.0522 mmol) was added to a solution of 1-(2-methoxyethyl)piperazine (5.65 mg, 0.0392 mmol) and N-[5-(1H-benzimidazol-2-yl)-1-(2-hydroxyethyl)pyrazol-3-yl]-6-chloro-pyridine-3-carboxamide (A-196, Step 1) (10 mg, 0.0261 mmol) in DMF (1 mL). The mixture was heated at 100° C. for 20 h. The mixture was cooled to 23° C., and water (10 mL) was added. The aq. phase was extracted with EtOAc (3×10 mL). The combined organic phases were washed with brine (30 mL), dried (MgSO₄), filtered, and concentrated. The residue was purified by preparative HPLC (BEH C18 30×150) eluting with water 10 mM (NH₄)(HCO₃)) and MeCN (26-36%) to provide the title compound as a solid (6.00 mg, 47%). ¹H NMR (500 MHz, DMSO) δ 10.86 (s, 1H), 8.80 (d, J=2.6 Hz, 1H), 8.45-8.29 (m, 1H), 8.15 (dd, J=9.0, 2.5 Hz, 1H), 7.61 (s, 2H), 7.46 (s, 1H), 7.25 (dd, J=6.0, 3.1 Hz, 1H), 6.88 (d, J=9.1 Hz, 1H), 4.85 (t, J=6.1 Hz, 2H), 3.85 (t, J=6.2 Hz, 2H), 3.67-3.56 (m, 4H), 3.48 (t, J=5.8 Hz, 2H), 3.25 (s, 3H), 2.53 (m, 6H). m/z (ES⁻), [M−H]⁻ 490.2. HPLC (A05) t_{R}=2.16 min.

### Preparation of Compound A-200 (N-[5-(1H-benzimidazol-2-yl)-1-(2-hydroxyethyl)pyrazol-3-yl]-6-(4-methyl-piperazin-1-yl)pyridine-3-carboxamide)

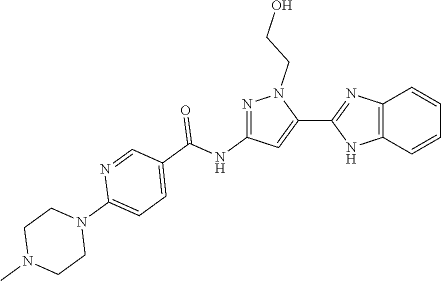 [SMILES: CN1CCN(c2ccc(C(=O)Nc3cc(-c4nc5ccccc5[nH]4)n(CCO)n3)cn2)CC1]

DIEA (0.00910 mL, 0.0522 mmol) was added to a solution of 1-methylpiperazine (3.92 mg, 0.0392 mmol) and N-[5-(1H-benzimidazol-2-yl)-1-(2-hydroxyethyl)pyrazol-3-yl]-6-chloro-pyridine-3-carboxamide (A-196, Step 1) (10 mg, 0.0261 mmol) in DMF (1 mL). The mixture was heated at 80° C. for 20 h. The mixture was cooled to 23° C., and water (10 mL) was added. The aq. phase was extracted with EtOAc (3×10 mL). The combined organic phases were washed with brine (10 mL), dried (MgSO₄), filtered, and concentrated. The residue was purified by preparative HPLC (BEH C18 30×150) eluting with water 10 mM (NH₄)(HCO₂)) and MeCN (33-43%) to provide the title compound as a solid (9.00 mg, 77%). ¹H NMR (400 MHz, DMSO) δ 10.94-10.79 (m, 1H), 8.80 (d, J=2.5 Hz, 1H), 8.15 (dd, J=9.0, 2.5 Hz, 1H), 7.62 (s, 2H), 7.46 (s, 1H), 7.25 (dd, J=6.1, 3.1 Hz, 2H), 6.89 (d, J=9.1 Hz, 1H), 4.85 (t, J=6.1 Hz, 2H), 3.85 (t, J=6.2 Hz, 4H), 3.65-3.60 (m, 4H), 2.39 (d, J=5.1 Hz, 2H), 2.22 (s, 3H). m/z (ES⁺), [M−H]⁻ 446.2. HPLC (A05) t_{R}=2.02 min.

### Preparation of Compound A-201.

### Step 1: N-[5-(1H-benzimidazol-2-yl)-1-(2-hydroxyethyl)pyrazol-3-yl]-4-fluoro-benzamide

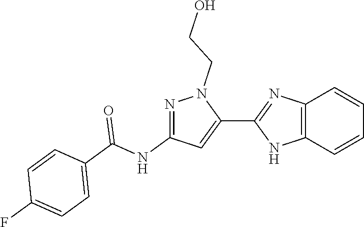 [SMILES: O=C(Nc1cc(-c2nc3ccccc3[nH]2)n(CCO)n1)c1ccc(F)cc1]

1,4-Dioxane (3 mL) was added to N-[1-allyl-5-(1H-benzimidazol-2-yl)pyrazol-3-yl]-4-fluoro-benzamide (L-26) (105 mg, 0.291 mmol). After cooling to 0° C., 2,6-lutidine (0.202 mL, 1.74 mmol) and NaIO₄ (373 mg, 1.74 mmol) were added. A solution of potassium osmate(IV) dihydrate (2.14 mg, 0.00581 mmol) in water (1 mL) was added. The mixture was heated at 23° C. for 18 h. Sat. Na₂S₂O₃ (50 mL) was added, and the aq. phase was extracted with EtOAc (3×10 mL). The combined organic phases were washed with brine (40 mL), dried (MgSO₄), filtered, and concentrated. The residue (m/z (ES⁺), [M−H]⁻ 362.6. HPLC (A05) t_{R}=2.06 min) was suspended in MeOH (4 mL), and NaBH₄ (16.5 mg, 0.436 mmol) was added. The mixture was stirred at 40° C. for 1 h. The mixture was cooled to 23° C., and water (10 mL) was added. The aq, phase was extracted with EtOAc (4×20 mL). The combined organic phases were washed with brine (10.0 mL), dried (MgSO₄), filtered, and concentrated to provide the title compound as a solid (44 mg, 41%). ¹H NMR (400 MHz, DMSO) δ 13.11-13.03 (m, 1H), 11.15 (s, 1H), 8.13 (dd, J=9.0, 5.5 Hz, 2H), 7.72 (s, 1H), 7.57-7.50 (m, 1H), 7.49 (s, 1H), 7.35 (dd, J=11.2, 6.5 Hz, 2H), 7.25 (s, 2H), 5.08-4.92 (m, 1H), 4.87 (t, J=6.1 Hz, 2H), 3.85 (s, 2H). m/z (ES), [M−H]⁻ 364.6. HPLC (A05) t_{R}=2.14 min.

### Step 2: N-[5-(1H-benzimidazol-2-yl)-1-(2-hydroxyethyl)pyrazol-3-yl]-4-(4-methylpiperazin-1-yl)benzamide

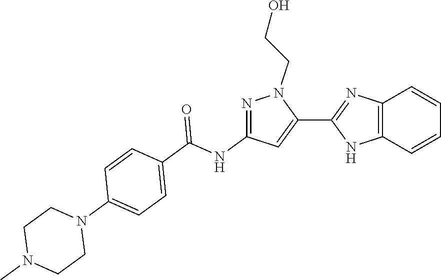 [SMILES: CN1CCN(c2ccc(C(=O)Nc3cc(-c4nc5ccccc5[nH]4)n(CCO)n3)cc2)CC1]

1-Methylpiperazine (1 mL, 9.02 mmol) was added to N-[5-(1H-benzimidazol-2-yl)-1-[(4-methoxyphenyl)methyl]pyrazol-3-yl]-4-fluoro-benzamide (42 mg, 0.115 mmol) in DMSO (0.100 mL). The mixture was stirred at 120° C. for 48 h. The mixture was cooled to 23° C., and water (40 mL) was added. The aq. phase was extracted with EtOAc (3×10 mL). The combined organic phases were washed with brine (50 mL), dried (MgSO₄), filtered, and concentrated. The residue was purified by preparative HPLC (BEH C18 30×150) eluting with water (10 mM (NH₄)(HCO₂)) and MeCN (21-31%) to provide the title compound as a solid (35.6 mg, 70%). ¹H NMR (400 MHz, DMSO) δ 10.75 (s, 1H), 8.30 (s, 2H), 7.96 (d, J=9.0 Hz, 2H), 7.47 (s, 1H), 7.28-7.22 (m, 2H), 6.98 (d, J=9.1 Hz, 2H), 4.85 (t, J=6.2 Hz, 2H), 3.85 (t, J=6.2 Hz, 2H), 3.31-3.26 (m, 4H), 2.47-2.42 (m, 4H), 2.23 (s, 3H). m/z (ES⁺), [M+H]⁺ 445.2. HPLC (A05) t_{R}=2.10 min.

### EXAMPLE B31

### Preparation of Compounds A-197 and A-199

### Representative Scheme for Synthesis of Compounds A-197 and A-199.

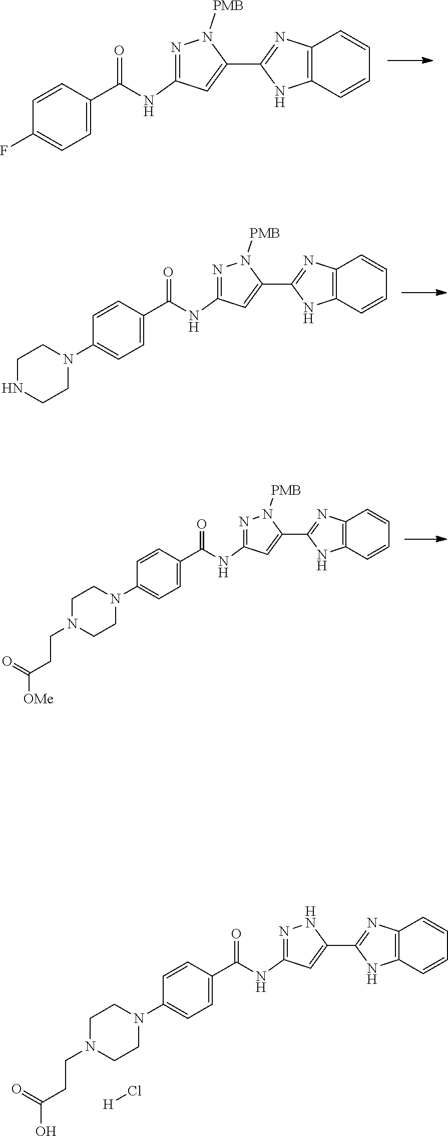

### Preparation of Compound A-197.

### Step 1: N-[5-(1H-benzimidazol-2-yl)-1-[(4-methoxyphenyl)methyl]pyrazol-3-yl]-4-piperazin-1-yl-benzamide

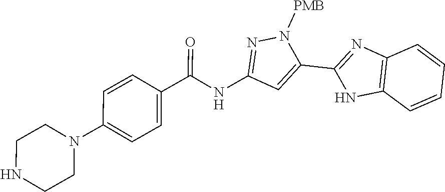

N-[5-(1H-Benzimidazol-2-yl)-1-[(4-methoxyphenyl)methyl]pyrazol-3-yl]-4-fluoro-benzamide (L-15) (500 mg, 1.13 mmol) was added to a solution of piperazine (2.00 g, 23.2 mmol) in DMSO (5 mL). The mixture was heated at 120° C. for 18 h. EtOAc (300 mL) was added, and the organic phase was washed with water (3×100 mL) and brine (80 mL), dried (Na₂SO₄), filtered, and concentrated to provide the title compound as a solid (556 mg, 97%). ¹H NMR (400 MHz, DMSO) δ 13.11 (s, 1H), 10.74 (s, 1H), 7.94 (d, J=9.0 Hz, 2H), 7.71 (d, J=8.5 Hz, 1H), 7.57-7.49 (m, 2H), 7.24 (dd, J=13.2, 10.2 Hz, 4H), 6.94 (d, J=9.1 Hz, 2H), 6.87-6.82 (m, 2H), 6.02 (s, 2H), 3.68 (s, 3H), 3.22-3.15 (m, 4H), 2.84-2.78 (m, 4H), 2.35 (s, 1H). m/z (ES⁺), [M+H]⁺ 508.8. HPLC (A05) t_{R}=2.00 min.

### Step 2: methyl 3-[4-[4-[[5-(1H-benzimidazol-2-yl)-1-[(4-methoxyphenyl)-methyl]pyrazol-3-yl]carbamoyl]phenyl]piperazin-1-yl]propanoate

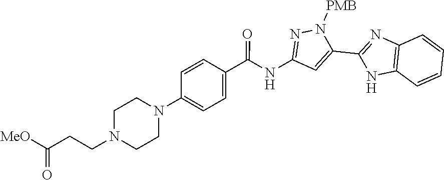

Methyl prop-2-enoate (0.0179 mL, 0.197 mmol) was added to a solution of N-[5-(1H-benzimidazol-2-yl)-1-[(4-methoxyphenyl)methyl]pyrazol-3-yl]-4-piperazin-1-yl-benzamide (100 mg, 0.197 mmol) in EtOH (2 mL). The mixture was heated at 50° C. for 18 h and concentrated. The residue was purified by silica gel chromatography (12 g cartridge) eluting with hexanes and EtOAc (0-100%) to provide the title compound as a solid (86 mg, 74%). ¹H NMR (400 MHz, DMSO) δ 13.08 (s, 1H), 10.72 (s, 1H), 7.91 (d, J=9.0 Hz, 2H), 7.68 (dd, J=21.7, 9.9 Hz, 1H), 7.55-7.44 (m, 2H), 7.26-7.13 (m, 4H), 6.93 (d, J=9.1 Hz, 2H), 6.84-6.77 (m, 2H), 5.99 (s, 2H), 3.64 (s, 3H), 3.56 (s, 3H), 3.25-3.19 (m, 4H), 2.58 (t, J=6.9 Hz, 2H), 2.51-2.47 (m, 6H). m/z (ES⁺), [M+H]⁺ 594.4. HPLC (A05) t_{R}=2.21 min.

### Step 3: 3-[4-[4-[[5-(1H-benzimidazol-2-yl)-1H-pyrazol-3-yl]carbamoyl]phenyl]-piperazin-1-yl]propanoic acid hydrochloride

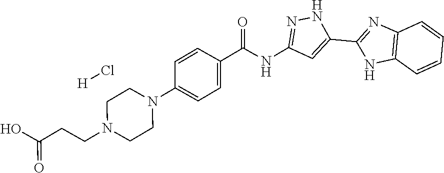

TFA (2 mL) was added to methyl 3-[4-[4-[[5-(1H-benzimidazol-2-yl)-1-[(4-methoxyphenyl)methyl]pyrazol-3-yl]carbamoyl]phenyl]piperazin-1-yl]propanoate (86 mg, 0.145 mmol), and the mixture was stirred at 23° C. for 1 h. The mixture was concentrated, and the residue was dissolved in THF (2 mL), MeOH (2 mL), and water (2 mL). LiOH—H₂O (0.0304 g, 0.724 mmol) was added, and the mixture was stirred at 23° C. for 18 h. The mixture was concentrated, and water (10 mL) was added. The aq. phase was acidified to pH 2 and lyophilized. Water (10 mL) was added to the residue. The mixture was filtered, and the solid was washed with water (10 mL), acetone (10 mL), DCM (10 mL), and acetone (10 mL) and dried to provide the title compound as a solid (65 mg, 90%). ¹H NMR (400 MHz, DMSO) δ 13.14 (s, 1H), 11.04 (s, 2H), 10.56 (s, 1H), 8.01 (d, J=8.9 Hz, 2H), 7.71-7.60 (m, 2H), 7.40-7.27 (m, 2H), 7.13 (d, J=8.9 Hz, 2H), 6.82 (s, 1H), 4.04 (s, 1H), 3.73-3.48 (m, 4H), 3.42-3.32 (m, 2H), 3.29-3.04 (m, 4H), 2.86 (t, J=7.6 Hz, 2H). m/z (ES), [M−H−HCl]⁻ 558.7. HPLC (A05) t_{R}=1.77 min.

### Preparation of Compound A-199.

### Step 1: ethyl 2-[4-[4-[[5-(1H-benzimidazol-2-yl)-1-[(4-methoxyphenyl)-methyl]pyrazol-3-yl]carbamoyl]phenyl]piperazin-1-yl]acetate

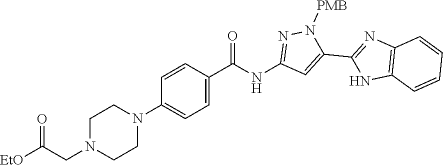

Ethyl 2-bromoacetate (0.0218 mL, 0.197 mmol) was added to a solution of N-[5-(1H-benzimidazol-2-yl)-1-[(4-methoxyphenyl)methyl]pyrazol-3-yl]-4-piperazin-1-yl-benzamide (A-197, Step 1) (100 mg, 0.197 mmol) in DMF (2 mL). The solution was stirred at 23° C. for 10 min. The mixture was concentrated, and EtOAc (100 mL) was added. The organic phase was washed with water (3×50 mL) and brine (50 mL), dried (Na₂SO₄), filtered, and concentrated. The residue was purified by silica gel chromatography (12 g cartridge) eluting with hexanes and EtOAc (0-100%) to provide the title compound as a solid (64 mg, 55%). ¹H NMR (400 MHz, DMSO) δ 13.11 (s, 1H), 10.76 (s, 1H), 7.95 (d, J=9.0 Hz, 2H), 7.71 (d, J=5.8 Hz, 1H), 7.56-7.49 (m, 2H), 7.26 (dd, J=22.5, 7.6 Hz, 4H), 6.97 (d, J=9.2 Hz, 2H), 6.87-6.83 (m, 2H), 6.02 (s, 2H), 4.10 (q, J=7.1 Hz, 2H), 3.68 (s, 3H), 3.31-3.27 (m, 6H), 2.67-2.62 (m, 4H), 1.20 (t, J=7.1 Hz, 3H). m/z (ES⁺), [M+H]⁺ 594.3. HPLC (A05) t_{R}=2.47 min.

### Step 2: 2-[4-[4-[[5-(1H-benzimidazol-2-yl)-1H-pyrazol-3-yl]carbamoyl]phenyl]-piperazin-1-yl]acetic acid hydrochloride

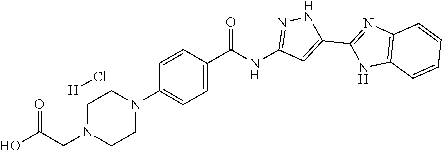

TFA (1 mL) was added to ethyl 2-[4-[4-[[5-(1H-benzimidazol-2-yl)-1-[(4-methoxyphenyl)methyl]pyrazol-3-yl]carbamoyl]phenyl]piperazin-1-yl]acetate (63 mg, 0.106 mmol). The mixture was stirred at 23° C. for 1 h and concentrated. THF (1 mL), MeOH (2 mL), water (1 mL), and LiOH—H₂O (40 mg, 0.953 mmol) were added to the residue, and the mixture was heated at 50° C. for 18 h. The mixture was concentrated, and water (4 mL) was added. The mixture was acidified to pH 2 with 1M HCl. The mixture was filtered, and the solid was purified by preparative HPLC (BEH C18 30×100) eluting with water (0.1% TFA) and MeCN (5-20%). 1M HCl (4 mL) was added to the purified residue, and the suspension was sonicated until fully dissolved. Acetone (8 mL) was added. The mixture was filtered, and the solid was washed with water (10 mL) and acetone (10 mL) to provide the title compound as a solid (5.10 mg, 10%). ¹H NMR (400 MHz, DMSO) δ 11.11 (s br, 2H), 10.57-10.19 (s br, 1H), 8.01 (d, J=8.9 Hz, 2H), 7.75-7.67 (m, 2H), 7.50-7.36 (m, 2H), 7.18-7.06 (m, 3H), 4.19 (s, 2H), 4.07-3.57 (m, 4H), 3.30-3.08 (m, 4H). m/z (ES⁺), [M+H]⁺ 446.4. HPLC (A05) t_{R}=1.79 min.

### EXAMPLE B32

### Preparation of Compound A-10

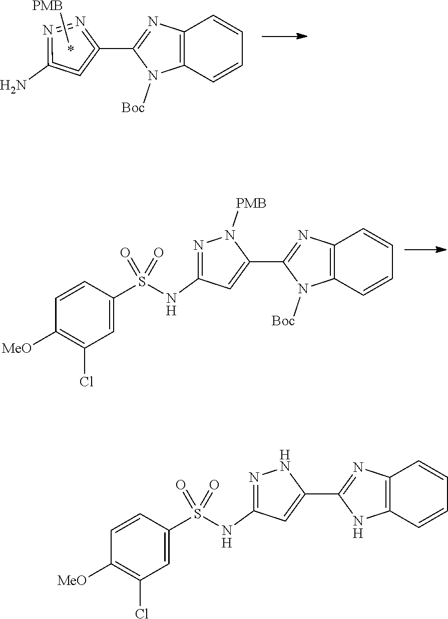

### Step 1: tert-butyl 2-[5-[(3-chloro-4-methoxy-phenyl)sulfonylamino]-2-[(4-methoxyphenyl)methyl]pyrazol-3-yl]benzimidazole-1-carboxylate

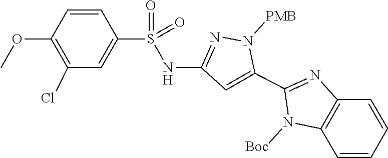

3-Chloro-4-methoxy-benzenesulfonyl chloride (50 mg, 0.207 mmol) was dissolved in DCM (2 mL) and cooled to 0° C. DIEA (36.1 μL, 0.207 mmol) was added, and the mixture was stirred at 0° C. for 10 min. A 3:1 mixture of tert-butyl 2-[5-amino-2- and tert-butyl 2-[5-amino-1-[(4-methoxyphenyl)methyl]pyrazol-3-yl]benzimidazole-1-carboxylate (A-2, Step 3) (113 mg, 0.270 mmol) was added. The mixture was warmed to 40° C. and stirred for 18 h. Brine (10 mL) and DCM (10 mL) were added. The organic phase was separated, and the aq. phase was extracted with DCM (2×10 mL). The combined organic phases were dried (MgSO₄), filtered, and concentrated. The product was purified by reverse phase chromatography (C18, 54 g cartridge) eluting with water (10 mM (NH₄)(HCO₂)) and MeCN (5-100%) to provide the title compound as a solid (56.3 mg; purity 65%, 28%). ¹H NMR (500 MHz, DMSO) δ 10.72 (s, 1H), 8.06-8.02 (m, 1H), 7.85 (d, J=2.3 Hz, 1H), 7.83-7.80 (m, 1H), 7.78 (dd, J=8.7, 2.3 Hz, 1H), 7.53-7.47 (m, 1H), 7.46-7.41 (m, 1H), 7.30 (d, J=8.9 Hz, 1H), 6.92-6.87 (m, 2H), 6.75-6.71 (m, 2H), 6.38 (s, 1H), 5.11 (s, 2H), 3.94 (s, 3H), 3.67 (s, 3H), 1.27 (s, 9H). m/z (ES), [M−H]⁻ 622.9. HPLC (A05) t_{R}=2.58 min.

### Step 2: N-[5-(1H-benzimidazol-2-yl)-1H-pyrazol-3-yl]-3-chloro-4-methoxy-benzenesulfonamide

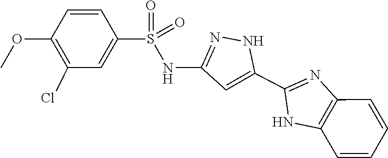 [SMILES: COc1ccc(S(=O)(=O)Nc2cc(-c3nc4ccccc4[nH]3)[nH]n2)cc1Cl]

tert-Butyl 2-[5-[(3-chloro-4-methoxy-phenyl)sulfonylamino]-2-[(4-methoxyphenyl)methyl]-pyrazol-3-yl]benzimidazole-1-carboxylate (65.0%, 25 mg, 0.0260 mmol) was dissolved in DCM (0.500 mL). TFA (0.500 mL) was added, and the mixture was stirred at 23° C. for 2 h. The mixture was concentrated. The product was purified by HPLC (BEH C₁₈ 30×100 mm) eluting with water (10 mM (NH₄)(HCO₂)) and MeCN (33-53%). The purified residue was dissolved in EtOAc (10 mL), and the mixture was washed with water (10 mL). The organic phase was concentrated and dried to provide the title compound as a solid (7.84 mg, 75%). ¹H NMR (400 MHz, DMSO) δ 13.46 (s, 1H), 12.96 (s, 1H), 10.67 (s, 1H), 7.84 (d, J=2.3 Hz, 1H), 7.75 (dd, J=8.8, 2.3 Hz, 1H), 7.65 (d, J=7.4 Hz, 1H), 7.54 (d, J=7.2 Hz, 1H), 7.31 (d, J=8.9 Hz, 1H), 7.29-7.18 (m, 2H), 6.79 (d, J=1.9 Hz, 1H), 3.92 (s, 3H). m/z (ES), [M−H]⁻ 402.2. HPLC (A05) t_{R}=2.17 min.

### EXAMPLE B33

### Preparation of Compound A-11

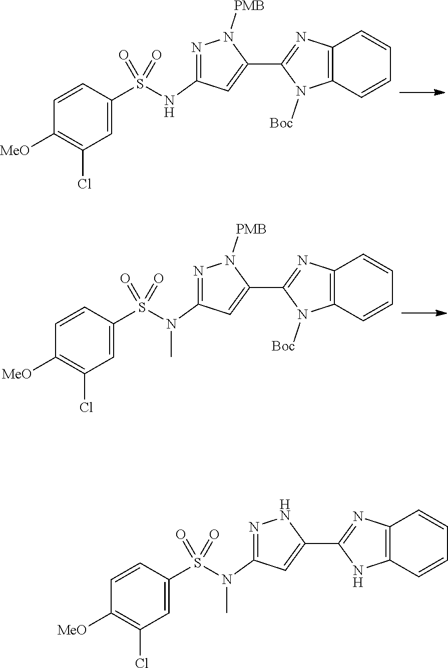

### Step 1: tert-butyl 2-[5-[(3-chloro-4-methoxy-phenyl)sulfonyl-methyl-amino]-2-[(4-methoxyphenyl)methyl]pyrazol-3-yl]benzimidazole-1-carboxylate

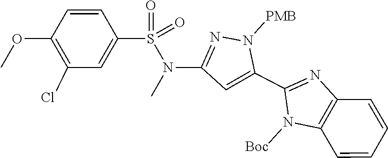

DMF (1 mL) was added to a mixture of tert-butyl 2-[5-[(3-chloro-4-methoxy-phenyl)sulfonylamino]-2-[(4-methoxyphenyl)methyl]pyrazol-3-yl]benzimidazole-1-carboxylate (A-10, Step 1) (65%, 29.4 mg, 0.0306 mmol) and Cs₂CO₃ (29.9 mg, 0.0919 mmol). MeI (3.81 μL, 0.0612 mmol) was added, and the mixture was stirred at 23° C. for 2.5 h. The mixture was concentrated. Brine (10 mL) was added, and the aq. phase was extracted with EtOAc (3×10 mL). The combined organic phases were dried (MgSO₄), filtered, and concentrated. The product was purified by silica gel chromatography (7 g cartridge) eluting with hexanes and EtOAc (0-50%) to provide the title compound as a solid (12.9 mg; 66%). ¹H NMR (500 MHz, DMSO) δ 8.06-8.02 (m, 1H), 7.85-7.81 (m, 1H), 7.74 (d, J=2.3 Hz, 1H), 7.69 (dd, J=8.7, 2.3 Hz, 1H), 7.54-7.49 (m, 1H), 7.48-7.43 (m, 1H), 7.29 (d, J=8.8 Hz, 1H), 6.91-6.85 (m, 2H), 6.77-6.72 (m, 2H), 6.70 (s, 1H), 5.19 (s, 2H), 3.96 (s, 3H), 3.67 (s, 3H), 3.19 (s, 3H), 1.38 (s, 9H). m/z (ES⁺), [M+H]⁺ 638.5. HPLC (A05) t_{R}=2.92 min.

### Step 2: N-[5-(1H-benzimidazol-2-yl)-1H-pyrazol-3-yl]-3-chloro-4-methoxy-N-methyl-benzenesulfonamide

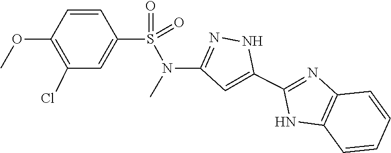 [SMILES: COc1ccc(S(=O)(=O)N(C)c2cc(-c3nc4ccccc4[nH]3)[nH]n2)cc1Cl]

tert-Butyl 2-[5-[(3-chloro-4-methoxy-phenyl)sulfonyl-methyl-amino]-2-[(4-methoxyphenyl)-methyl]pyrazol-3-yl]benzimidazole-1-carboxylate (11.8 mg, 0.0185 mmol) was dissolved in DCM (0.500 mL). TFA (0.500 mL) was added, and the mixture was stirred at 23° C. for 3 h. The mixture was concentrated. Sat. NaHCO₃ (10 mL) was added, and the aq. phase was extracted with EtOAc (3×10 mL). The combined organic phases were washed with brine (10 mL), dried (MgSO₄), filtered, and concentrated. The product was purified by silica gel chromatography (7 g cartridge) eluting with hexanes and EtOAc (0-100%) to provide the title compound as a solid (4.81 mg, 62%). ¹H NMR (500 MHz, DMSO) δ 13.67 (s, 1H), 13.07 (s, 1H), 7.69 (d, J=2.3 Hz, 1H), 7.68-7.62 (m, 2H), 7.62-7.55 (m, 1H), 7.34 (d, J=8.7 Hz, 1H), 7.30-7.21 (m, 2H), 7.09 (s, 1H), 3.94 (s, 3H), 3.21 (s, 3H). m/z (ES+), [M+H]⁺ 418.3. HPLC (A05) t_{R}=2.34 min.

### EXAMPLE B34

### Preparation of Compound A-15

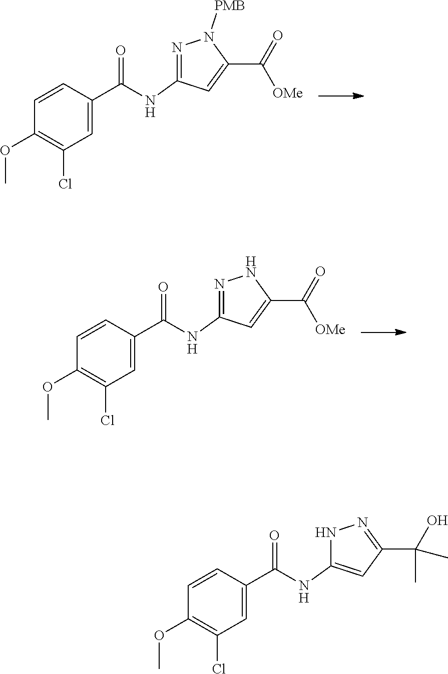

### Step 1: methyl 3-[(3-chloro-4-methoxy-benzoyl)amino]-1H-pyrazole-5-carboxylate

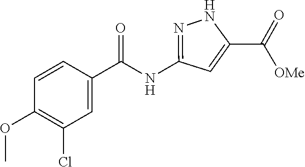 [SMILES: COC(=O)c1cc(NC(=O)c2ccc(OC)c(Cl)c2)n[nH]1]

TFA (0.500 mL) was added to methyl 5-[(3-chloro-4-methoxy-benzoyl)amino]-2-[(4-methoxyphenyl)methyl]pyrazole-3-carboxylate (M-2, Step 1) (61.6 mg, 0.143 mmol), and the mixture was heated at 70° C. for 30 min. After cooling to 23° C., the mixture was concentrated. Sat. NaHCO₃ (15 mL) was added, and the aq. phase was extracted with EtOAc (3×15 mL). The combined organic phases were washed with brine (15 mL), dried (MgSO₄), filtered, and concentrated. The residue was diluted in hexanes (10 mL). The solid was filtered and dried under high vacuum to provide the title compound as a solid in a 3:1 mixture of pyrazole tautomers (32.5 mg, 73%). ¹H NMR (500 MHz, DMSO) δ 13.69 [13.29] (s, 1H), 11.03 [11.06] (s, 1H), 8.14 [8.11-8.07] (s, 1H), 8.04 [7.99] (d, J=9.5 Hz, 1H), 7.27 [7.37-7.31] (d, J=8.4 Hz, 1H), 7.14 [7.04] (s, 1H), 3.95 [3.95] (s, 3H), 3.87 [3.81] (s, 3H). m/z (ES⁺), [M+H]⁺ 310.1. HPLC (A05) t_{R}=2.12 min.

### Step 2: 3-chloro-N-[3-(1-hydroxy-1-methyl-ethyl)-1H-pyrazol-5-yl]-4-methoxy-benzamide

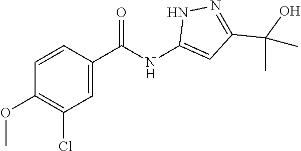 [SMILES: COc1ccc(C(=O)Nc2cc(C(C)(C)O)n[nH]2)cc1Cl]

3.0 M MeMgBr in Et₂O (0.108 mL, 0.323 mmol) was added dropwise at 0° C. to a solution of methyl 3-[(3-chloro-4-methoxy-benzoyl)amino]-1H-pyrazole-5-carboxylate (20 mg, 0.0646 mmol) in THF (0.650 mL). The mixture was warmed to 23° C. and stirred for 2 h. Sat. NH₄Cl (10 mL) was added dropwise. The aq. phase was extracted with EtOAc (3×10 mL). The combined organic phases were washed with brine (10 mL), dried (MgSO₄), filtered, and concentrated. The product was purified by silica gel chromatography eluting with mixtures of DCM and MeOH (0-5%) to provide the title compound as a solid (5.27 mg, 26%). ¹H NMR (400 MHz, DMSO) δ 12.09 (s, 1H), 10.61 (s, 1H), 8.06 (d, J=2.2 Hz, 1H), 7.96 (dd, J=8.7, 2.2 Hz, 1H), 7.20 (d, J=8.8 Hz, 1H), 6.42 (s, 1H), 5.16 (s, 1H), 3.88 (s, 3H), 1.41 (s, 6H). m/z (ES⁺), [M−H₂O+H]⁺: 292.2. HPLC (A05) t_{R}=1.98 min.

### EXAMPLE B35

### Preparation of Compound A-18

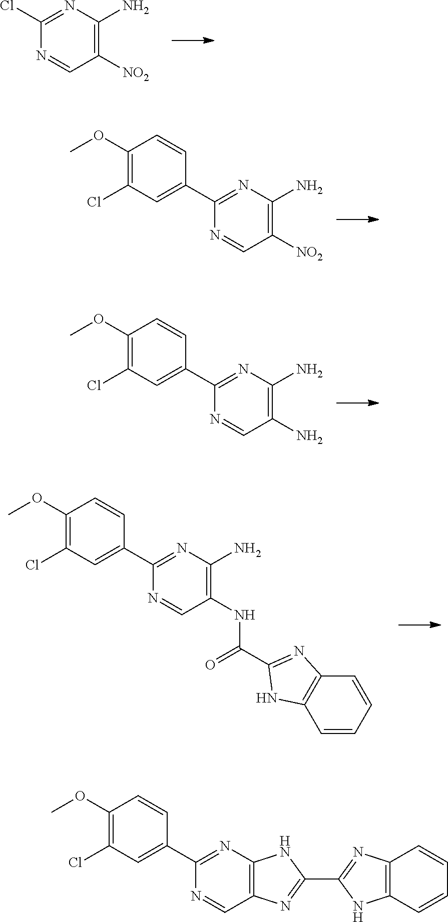 [SMILES: Nc1nc(Cl)ncc1[N+](=O)[O-] ; COc1ccc(-c2ncc([N+](=O)[O-])c(N)n2)cc1Cl ; COc1ccc(-c2ncc(N)c(N)n2)cc1Cl ; COc1ccc(-c2ncc(NC(=O)c3nc4ccccc4[nH]3)c(N)n2)cc1Cl ; COc1ccc(-c2ncc3nc(-c4nc5ccccc5[nH]4)[nH]c3n2)cc1Cl]

### Step 1: 2-(3-chloro-4-methoxy-phenyl)-5-naro-pyrimidin-4-amine

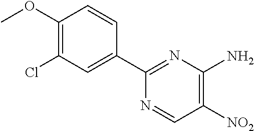 [SMILES: COc1ccc(-c2ncc([N+](=O)[O-])c(N)n2)cc1Cl]

Nitrogen was bubbled through a mixture of 2-chloro-5-nitro-pyrimidin-4-amine (0.873 g, 5.00 mmol), (3-chloro-4-methoxy-phenyl)boronic acid (1.30 g, 7.00 mmol), Cs₂CO₃ (3.26 g, 10.0 mmol), and Pd(dppf)Cl₂-DCM (0.204 g, 0.250 mmol) in 1,4-dioxane (50 mL) and water (10 mL). The mixture was heated at 90° C. for 3 h. After cooling to 23° C., the mixture was filtered through Celite washing with EtOAc (3×50 mL). The filtrate was washed with 1N aq. NaOH (3×50 mL) and brine (50 mL), dried (MgSO₄), filtered, and concentrated. The residue was diluted in MeCN (50 mL) and stirred for 10 min. The mixture was filtered, and the solid was dried under high vacuum to provide the title compound as a solid (549 mg; 39%). ¹H NMR (400 MHz, DMO) δ 9.19 (s, 1H), 8.79 (br, 1H), 8.39 (d, J=2.1 Hz, 1H), 8.36 (br, 1H), 8.35 (dd, J=8.7, 2.1 Hz, 1H), 7.34 (d, J=8.8 Hz, 1H), 3.97 (s, 3H). m/z (ES⁺), [M+H]⁺ 281.1. HPLC (B05) t_{R}=2.62 min.

### Step 2: 2-(3-chloro-4-methoxy-phenyl)pyrimidine-4,5-diamine

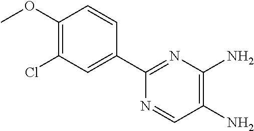 [SMILES: COc1ccc(-c2ncc(N)c(N)n2)cc1Cl]

AcOH (25 mL) was added to a mixture of iron powder (1.09 g, 19.5 mmol) and 2-(3-chloro-4-methoxy-phenyl)-5-nitro-pyrimidin-4-amine (0.547 g, 1.95 mmol). The mixture was stirred at 23° C. for 3 h. The mixture was filtered through Celite washing with EtOAc (3×40 mL). The filtrate was concentrated. Sat. NaHCO₃ (25 mL) was added dropwise, and the mixture was stirred at 23° C. for 5 min. Sat. Rochelle salt (25 mL) was added. The aq. phase was extracted with EtOAc (3×30 mL). The combined organic phases were washed with sat. NaHCO₃ (30 mL), sat. Rochelle salt (30 mL) and brine (30 mL). The organic phases were dried (MgSO₄), filtered, and concentrated. The residue was diluted in Et20 (50 mL). The mixture was filtered, and the solid was washed with hexanes and dried under high vacuum to provide the title compound as a solid (354 mg, 73%). ¹H NMR (500 MHz, DMSO) δ 8.17 (d, J=2.1 Hz, 1H), 8.09 (dd, J=8.6, 2.1 Hz, 1H), 7.66 (s, 1H), 7.17 (d, J=8.7 Hz, 1H), 6.40 (s, 2H), 4.84 (s, 2H), 3.89 (s, 3H). m/z (ES+), [M+H]⁺ 251.2. HPLC (A05) t_{R}=1.77 min.

### Step 3: N-[4-amino-2-(3-chloro-4-methoxy-phenyl)pyrimidin-5-yl]-1H-benzimidazole-2-carboxamide

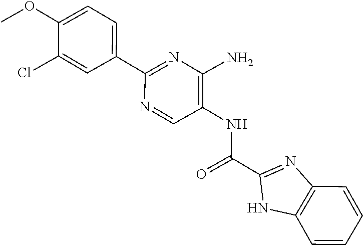 [SMILES: COc1ccc(-c2ncc(NC(=O)c3nc4ccccc4[nH]3)c(N)n2)cc1Cl]

HATU (0.152 g, 0.400 mmol) was added to a solution of 1H-benzimidazole-2-carboxylic acid (30 mg, 0.185 mmol) and DIEA (0.174 mL, 1.00 mmol) in DMF (2 mL) at 0° C. The mixture was stirred at 0° C. for 15 min. 2-(3-Chloro-4-methoxy-phenyl)pyrimidine-4,5-diamine (50.1 mg, 0.200 mmol) was added. The mixture was warmed to 23° C. and stirred for 18 h. Water (25 mL) was added, and the mixture was stirred at 23° C. for 5 min. The mixture was filtered, and the solid was purified by reverse phase chromatography (54 g, 2 runs) eluting with mixtures of water ((10 mM (NH₄)(HCO₃)) and MeCN (5-100%) to provide the title compound as a solid (33 mg, 42%). ¹H NMR (500 MHz, DMSO) δ 13.40 (s, 1H), 10.39 (s, 1H), 8.33 (d, J=2.1 Hz, 1H), 8.30 (s, 1H), 8.26 (dd, J=8.7, 2.1 Hz, 1H), 7.80 (d, J=7.2 Hz, 1H), 7.58 (d, J=7.9 Hz, 1H), 7.39-7.35 (m, 1H), 7.34-7.29 (m, 1H), 7.27 (d, J=8.9 Hz, 1H), 6.99 (s, 2H), 3.94 (s, 3H). m/z (ES⁺), [M+H]⁺ 395.3. HPLC (B05) t_{R}=2.41 min.

### Step 4: 8-(1H-benzimidazol-2-yl)-2-(3-chloro-4-methoxy-phenyl)-9H-purine

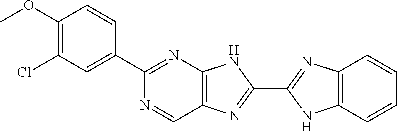 [SMILES: COc1ccc(-c2ncc3nc(-c4nc5ccccc5[nH]4)[nH]c3n2)cc1Cl]

A suspension of N-[4-amino-2-(3-chloro-4-methoxy-phenyl)pyrimidin-5-yl]-1H-benzimidazole-2-carboxamide (25 mg, 0.0633 mmol) in AcOH (1 mL) was heated at 140° C. under microwave irradiation for 6 h. Water (20 mL) was added. The mixture was filtered, and and the solid was washed with sat. NaHCO₃. The solid was diluted in MeOH. The mixture was stirred at 23° C. for 5 min and filtered. The filtrate was concentrated. The residue was purified by silica gel chromatography (4 g cartridge) eluting with mixtures of DCM and MeOH (0-10%) to provide the title compound as a solid (6.4 mg, 27%). ¹H NMR (400 MHz, DMSO) δ 14.51 (br, 1H), 13.76 (br, 1H), 9.23 (br, 1H), 8.47 (d, J=2.1 Hz, 1H), 8.43 (dd, J=8.6, 2.2 Hz, 1H), 7.81 (br, 1H), 7.63 (br, 1H), 7.34 (br, 2H), 7.34 (d, J=8.8 Hz, 1H), 3.97 (s, 3H). m/z (ES⁺), [M+H]⁺ 377.8. HPLC (B05) t_{R}=1.96 min.

### EXAMPLE B36

### Preparation of Compound A-19

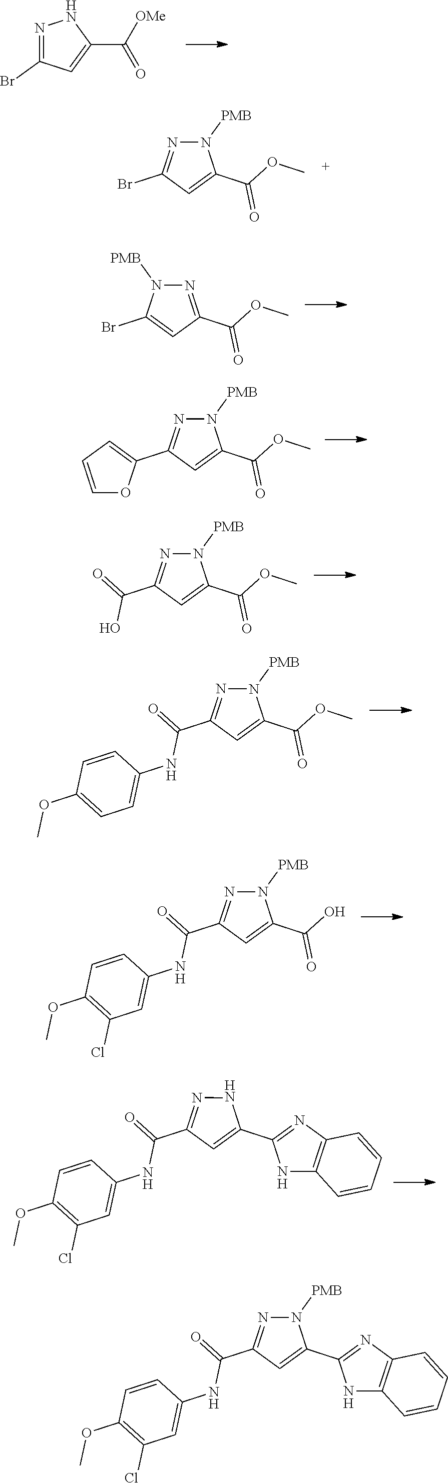

### Step 1: methyl 5-bromo-1 and methyl 5-bromo-2-[(4-methoxyphenyl)-methyl]pyrazole-3-carboxylate

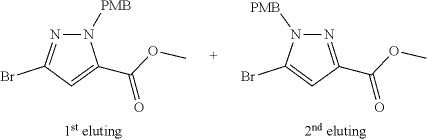

Methyl 5-bromo-1H-pyrazole-3-carboxylate (0.860 g, 4.19 mmol) and Cs₂CO₃ (2.734 g, 8.39 mmol) were dissolved in DMF (15 mL), and the solution was stirred at 23° C. for 10 min. 1-(Chloromethyl)-4-methoxybenzene (0.626 mL, 2.68 mmol) was added, and the mixture was stirred at 23° C. for 18 h. The mixture was concentrated. Water (40 mL) was added, and the aq. phase was extracted with EtOAc (3×60 mL). The combined organic phases were washed with brine (50 mL), dried (MgSO₄), filtered, and concentrated. The residue was purified by silica gel chromatography (40 g cartridge) eluting with hexanes and EtOAc (0-30%) to provide two compounds. 1^{st} eluting compound: methyl 5-bromo-2-[(4-methoxyphenyl)methyl]pyrazole-3-carboxylate as a solid (1.07 g, 79%). ¹H NMR (500 MHz, DMSO) δ 7.21-7.11 (m, 2H), 7.04 (s, 1H), 6.92-6.86 (m, 2H), 5.60 (s, 2H), 3.84 (s, 3H), 3.72 (s, 3H). ¹H NMR (500 MHz, CDCl₃) δ 7.30-7.26 (m, 2H), 6.86-6.82 (m, 2H), 6.82 (s, 1H), 5.65 (s, 2H), 3.86 (s, 3H), 3.77 (s, 3H). m/z (ES⁺), [M+Na]⁺ 349.9. HPLC (A05) t_{R}=2.57 min. 2^{nd} eluting isomer: methyl 5-bromo-1-[(4-methoxyphenyl)methyl]pyrazole-3-carboxylate as a solid (367 mg, 80% pure, 21%). ¹H NMR (500 MHz, DMSO) δ 7.18-7.14 (m, 2H), 6.99 (s, 1H), 6.95-6.90 (m, 2H), 5.39 (s, 2H), 3.80 (s, 3H), 3.73 (s, 3H). ¹H NMR (500 MHz, CDCl₃) δ 7.24-7.20 (m, 2H), 6.87-6.83 (m, 2H), 6.85 (s, 1H), 5.39 (s, 2H), 3.92 (s, 3H), 3.78 (s, 3H). m/z (ES⁺), [M+Na]⁺ 349.9. HPLC (A05) t_{R}=2.39 min.

### Step 2: methyl 5-(2-furyl)-2-[(4-methoxyphenyl)methyl]pyrazole-3-carboxylate

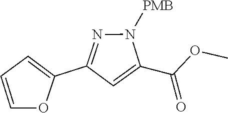

1,4-Dioxane (15 mL) and H₂O (3 mL) were sequentially added to a mixture of methyl 5-bromo-2-[(4-methoxyphenyl) methyl] pyrazole-3-carboxylate (400 mg, 1.23 mmol), 2-furyl-boronic acid (151 mg, 1.35 mmol), Pd(dppf)Cl₂ (90 mg, 0.123 mmol), and Na₂CO₃ (391 mg, 3.69 mmol) under nitrogen. The mixture was heated at 100° C. for 5 h. Water (25 mL) was added, and the aq. phase was extracted with EtOAc (3×40 mL). The combined organic phases were washed with brine (25 mL), dried (MgSO₄), filtered, and concentrated. The residue was purified by silica gel chromatography (24 g, cartridge) eluting with hexanes and EtOAc (0-25%) to provide the title compound as a solid (232 mg, 60%). ¹H NMR (400 MHz, CDCl₃) δ 7.46 (dd, J=1.8, 0.8 Hz, 1H), 7.30-7.26 (m, 2H), 7.05 (s, 1H), 6.86-6.79 (m, 2H), 6.69 (dd, J=3.4, 0.8 Hz, 1H), 6.47 (dd, J=3.4, 1.8 Hz, 1H), 5.72 (s, 2H), 3.86 (s, 3H), 3.76 (s, 3H). ES⁺ [M+H]⁺: 313.3; HPLC (A05); t_{R}=2.57 min.

### Step 3: 5-methoxycarbonyl-1-[(4-methoxyphenyl)methyl]pyrazole-3-carboxylic acid

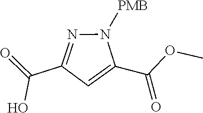

Methyl 5-(2-furyl)-2-[(4-methoxyphenyl) methyl] pyrazole-3-carboxylate (228 mg, 0.730 mmol) and NaIO₄ (625 mg, 2.92 mmol) were added to a mixture of EtOAc (3 mL) and MeCN (3 mL). A solution of RuCl₃·xH₂O (16.5 mg, 0.0730 mmol) in water (1 mL) was added, and the mixture was stirred at 23° C. for 1 h. Water (15 mL) was added, and the aq. phase was extracted with EtOAc (3×25 mL). The combined organic phases were washed with brine (25 mL), dried (MgSO₄), filtered, and concentrated. The residue was purified by silica gel chromatography (24 g, cartridge) eluting with hexanes and EtOAc (0-100%) to provide the title compound as a solid (72.5 mg, 34%). ¹H NMR (400 MHz, CDCl₃) δ 7.40 (s, 1H), 7.30 (d, J=8.8 Hz, 2H), 6.85-6.81 (m, 2H), 5.77 (s, 2H), 3.89 (s, 3H), 3.77 (s, 3H). ES− [M−H]⁻: 289.2; HPLC (A05); t_{R}=1.96 min.

### Step 4: methyl 5-[(3-chloro-4-methoxy-phenyl)carbamoyl]-2-[(4-methoxyphenyl)methyl]pyrazole-3-carboxylate

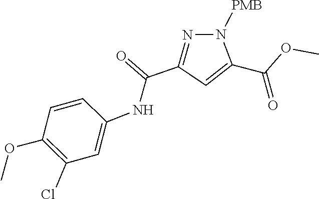

Oxalyl chloride (60.3 μL, 0.703 mmol) was added dropwise to a mixture of 5-methoxycarbonyl-1-[(4-methoxyphenyl)methyl]pyrazole-3-carboxylic acid (68 mg, 0.234 mmol) and DMF (1.80 μL, 23.4 μmol) in DCM (3 mL) at 0° C. The mixture was stirred at 0° C. for 1 h and concentrated. The residue was dissolved in DCM (3 mL), and pyridine (37.7 μL, 0.469 mmol) was added. A solution of 3-chloro-4-methoxy-aniline (44.3 mg, 0.281 mmol) in DCM (1 mL) was added dropwise. The mixture was stirred at 23° C. for 3 h and concentrated. Water (10 mL) was added, and the aq. was extracted with EtOAc (3×25 mL). The combined organic phases were washed with brine (15 mL), dried (MgSO₄), filtered, and concentrated. The residue was purified by silica gel chromatography (12 g, cartridge) eluting with hexanes and EtOAc (0-35%) to provide the title compound as a solid (66.4 mg, 66%). ¹H NMR (400 MHz, CDCl₃) δ 8.57 (s, 1H), 7.74 (d, J=2.6 Hz, 1H), 7.59 (dd, J=8.9, 2.6 Hz, 1H), 7.42 (s, 1H), 7.27-7.23 (m, 2H), 6.93 (d, J=8.9 Hz, 1H), 6.88-6.82 (m, 2H), 5.74 (s, 2H), 3.91 (s, 3H), 3.89 (s, 3H), 3.79 (s, 3H). ES⁺ [M+H]⁺: 430.3; HPLC (A05); t_{R}=2.62 min.

### Step 5: 5-[(3-chloro-4-methoxy-phenyl)carbamoyl]-2-[(4-methoxyphenyl)-methyl]pyrazole-3-carboxylic acid

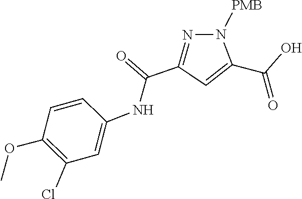

LiOH—H₂O (12.7 mg, 0.302 mmol) was added to a mixture of methyl 5-[(3-chloro-4-methoxy-phenyl)carbamoyl]-2-[(4-methoxyphenyl)methyl]pyrazole-3-carboxylate (65 mg, 0.151 mmol) in MeOH (1.60 mL), THF (1.60 mL) and water (0.500 mL). The mixture was heated at 70° C. for 1 h and concentrated. Water (5 mL) was added, and the aq. phase was acidified with HCl (1 mL, 1.00 M) dropwise. The mixture was filtered, and the solid was washed with hexanes and dried under high vacuum to provide the title compound as a solid (41.8 mg, 67%). m/z (ES⁺) [M+H]⁺: 416.3; HPLC (A05) t_{R}=2.16 min.

### Step 6: 5-(1H-benzimidazol-2-yl)-N-(3-chloro-4-methoxy-phenyl)-1-[(4-methoxyphenyl)methyl]pyrazole-3-carboxamide

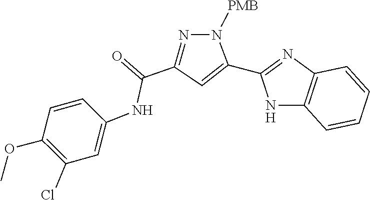

DIEA (58.5 μL, 0.336 mmol) was added to a solution of 5-[(3-chloro-4-methoxy-phenyl)carbamoyl]-2-[(4-methoxyphenyl)-methyl]pyrazole-3-carboxylic acid (38.8 mg, 0.0933 mmol) and o-phenylenediamine (10.6 mg, 0.0980 mmol) in DMF (3 mL). HATU (39 mg, 0.103 mmol) was added. The mixture was stirred at 23° C. for 2 h and concentrated. Water (20 mL) was added. The solid (m/z (ES⁺) [M+H]⁺: 506.1; HPLC (A05) t_{R}=2.54 min) was filtered and dried under high vacuum (45.6 mg). AcOH (2.50 mL) was added to the solid, and the mixture was heated at 75° C. for 3 h. The mixture was concentrated. Water (20 mL) and sat. NaHCO₃ (5 mL) were added. The mixture was filtered, and the solid was washed with water (20 mL) and dissolved in DCM (20 mL). The organic phase was dried (MgSO₄), filtered, and concentrated to provide the title compound as a solid (42.7 mg, 94%). m/z (ES⁺) [M+H]⁺: 489.2; HPLC (A05) t_{R}=2.69 min.

### Step 7: 5-(1H-benzimidazol-2-yl)-N-(3-chloro-4-methoxy-phenyl)-1H-pyrazole-3-carboxamide

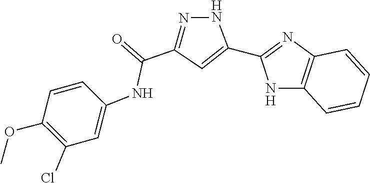 [SMILES: COc1ccc(NC(=O)c2cc(-c3nc4ccccc4[nH]3)[nH]n2)cc1Cl]

A solution of 5-(1H-benzimidazol-2-yl)-N-(3-chloro-4-methoxy-phenyl)-1-[(4-methoxyphenyl)-methyl]pyrazole-3-carboxamide (42.7 mg, 0.0875 mmol) in TFA (2 mL) was heated to 70° C. for 40 min. The mixture was concentrated. Sat. NaHCO₃ (10 mL) was added, and the aq. phase was extracted with EtOAc (3×30 mL). The combined organic phases were washed with brine (10 mL), dried (MgSO₄), filtered, and concentrated. The residue was purified by silica gel chromatography (4 g, cartridge) eluting with DCM and MeOH (0-15%) to provide the title compound as a solid in a 1:1 mixture of pyrazole tautomers (28.6 mg, 89%). ¹H NMR (500 MHz, CD₃OD) δ 7.87 (d, J=2.2 Hz, 1H), 7.67-7.57 (m, 3H), 7.54 (bs, 1H), 7.29 (bs, 2H), 7.10 (d, J=8.8 Hz, 1H), 3.90 (s, 3H). m/z (ES⁺) [M+H]⁺: 368.7; HPLC (A05) t_{R}=2.27 min.

### EXAMPLE B37

### Preparation of Compound A-27

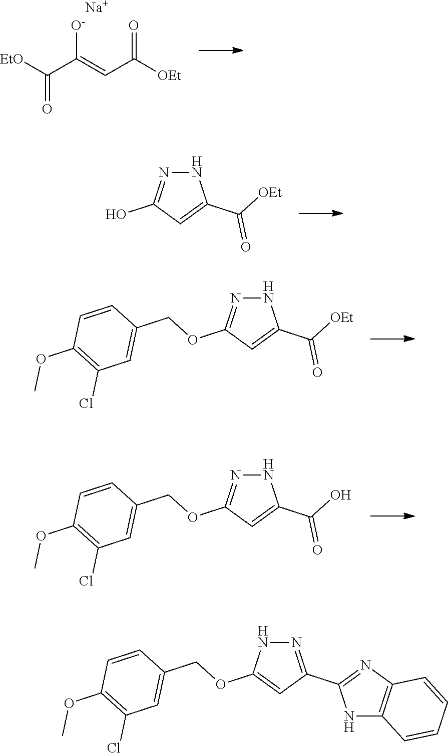 [SMILES: [Na+] ; CCOC(=O)/C=C(\[O-])C(=O)OCC ; CCOC(=O)c1cc(O)n[nH]1 ; CCOC(=O)c1cc(OCc2ccc(OC)c(Cl)c2)n[nH]1 ; COc1ccc(COc2cc(C(=O)O)[nH]n2)cc1Cl ; COc1ccc(COc2cc(-c3nc4ccccc4[nH]3)n[nH]2)cc1Cl]

### Step 1: N-[5-(1H-benzimidazol-2-yl)-1-[(4-methoxyphenyl)methyl]pyrazol-3-yl]-5,6-dichloro-pyridine-3-carboxamide

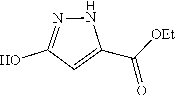 [SMILES: CCOC(=O)c1cc(O)n[nH]1]

Sulfuric acid (2.75 mL, 51.6 mmol) was added dropwise to a suspension of diethyl oxalacetate sodium salt (10.5 g, 50.0 mmol) in Et₂O (500 mL). The mixture was stirred at 23° C. for 20 min, and the solid was filtered. Sat. KHCO₃ (100 mL) was added slowly to the filtrate, and the mixture was vigorously stirred at 23° C. for 10 min. The organic phase was separated, dried (MgSO₄), filtered, and concentrated. EtOH (40 mL) was added to the residue. A solution of hydrazine hydrate (50%, 3.14 mL, 50.0 mmol) in EtOH (5 mL) was added dropwise. The mixture was heated at 95° C. for 30 min and concentrated. The residue was diluted in water (50 mL), and the mixture was filtered. The solid was dried to provide the title compound as a solid (3.74 g, 48%). ¹H NMR (400 MHz, DMSO) δ 12.77 (s, 1H), 9.94 (s, 1H), 5.97 (s, 1H), 4.33-4.17 (m, 2H), 1.28 (t, J=7.1 Hz, 3H). m/z (ES⁺), [M+H]⁺ 157.0. HPLC (A05) t_{R}=1.46 min.

### Step 2: ethyl 2-[(3-chloro-4-methoxy-phenyl)methyl]-5-hydroxy-pyrazole-3-carboxylate and ethyl 3-[(3-chloro-4-methoxy-phenyl)methoxy]-1H-pyrazole-5-carboxylate

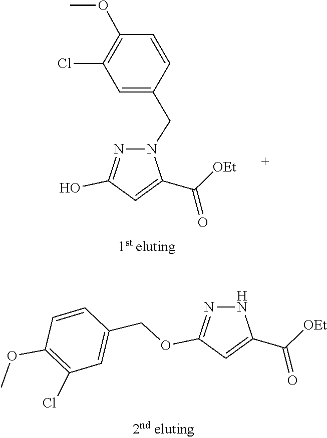 [SMILES: CCOC(=O)c1cc(O)nn1Cc1ccc(OC)c(Cl)c1 ; CCOC(=O)c1cc(OCc2ccc(OC)c(Cl)c2)n[nH]1]

DIAD (139 μL, 0.704 mmol) was added dropwise at 23° C. to a mixture of ethyl 3-hydroxy-1H-pyrazole-5-carboxylate (0.100 g, 0.640 mmol), (3-chloro-4-methoxy-phenyl)methanol (91.1 μL, 0.672 mmol), and PPh₃ (0.252 g, 0.961 mmol) in THF (3 mL). The mixture was stirred at 23° C. for 1 h and concentrated. The residue was purified by silica gel chromatography (25 g cartridge) eluting with mixtures of hexanes and EtOAc (0-40%), by reverse phase chromatography eluting with mixtures of water (10 mM (NH₄)(HCO₂)) and MeCN (5-100%), and by reverse phase chromatography eluting with mixtures of water (10 mM (NH₄)(HCO₃)) and MeCN (5-100%) to provide two compounds. 1^{st} eluting compound: ethyl 2-[(3-chloro-4-methoxy-phenyl)methyl]-5-hydroxy-pyrazole-3-carboxylate as a solid (23.3 mg, 12%). ¹H NMR (500 MHz, DMSO) δ 10.21 (s, 1H), 7.24 (s, 1H), 7.15-7.07 (m, 2H), 6.07 (s, 1H), 5.42 (s, 2H), 4.27 (q, J=7.1 Hz, 2H), 3.83 (s, 3H), 1.27 (t, J=7.1 Hz, 3H). m/z (ES⁺), [M+H]⁺ 311.2. HPLC (A05) t_{R}=2.32 min. 2^{nd} eluting compound: ethyl 3-[(3-chloro-4-methoxy-phenyl)methoxy]-1H-pyrazole-5-carboxylate as a solid (53.2 mg, 27%). ¹H NMR (500 MHz, DMSO) δ 13.16 (s, 1H), 7.51 (d, J=1.8 Hz, 1H), 7.39 (d, J=8.3 Hz, 1H), 7.15 (d, J=8.4 Hz, 1H), 6.29 (d, J=2.2 Hz, 1H), 5.11 (s, 2H), 4.29 (q, J=7.1 Hz, 2H), 3.86 (s, 3H), 1.29 (t, J=7.1 Hz, 3H). m/z (ES⁺), [M+H]⁺ 311.2. HPLC (A05) t_{R}=2.40 min.

### Step 3: 3-[(3-chloro-4-methoxy-phenyl)methoxy]-1H-pyrazole-5-carboxylic acid

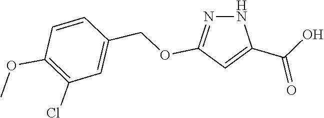 [SMILES: COc1ccc(COc2cc(C(=O)O)[nH]n2)cc1Cl]

6N NaOH in water (0.135 mL, 0.810 mmol) was added to a solution of ethyl 3-[(3-chloro-4-methoxy-phenyl)methoxy]-1H-pyrazole-5-carboxylate (50 mg, 0.161 mmol) in MeOH (1.25 mL), and the mixture was heated at 70° C. for 3 h. After cooling to 23° C., the mixture was concentrated. Water (5 mL) was added, and the aq. phase was acidified to pH 2 with conc. HCl. The mixture was filtered, and the solid was washed with water and dried under high vacuum to provide the title compound as a solid (37.5 mg, 82%). ¹H NMR (400 MHz, DMSO) δ 13.22 (br, 1H), 13.01 (br, 1H), 7.51 (s, 1H), 7.39 (d, J=8.0 Hz, 1H), 7.15 (d, J=8.5 Hz, 1H), 6.21 (s, 1H), 5.09 (s, 2H), 3.86 (s, 3H). m/z (ES⁺), [M+H]⁺ 283.1. HPLC (A05) t_{R}=1.90 min.

### Step 4: 2-[5-[(3-chloro-4-methoxy-phenyl)methoxy]-1H-pyrazol-3-yl]-1H-benzimidazole

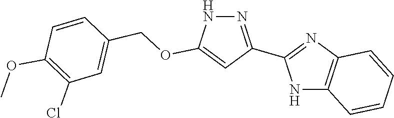 [SMILES: COc1ccc(COc2cc(-c3nc4ccccc4[nH]3)n[nH]2)cc1Cl]

DIEA (77.2 μL, 0.443 mmol) was added to a solution of HATU (51.5 mg, 0.135 mmol) and 3-[(3-chloro-4-methoxy-phenyl)methoxy]-1H-pyrazole-5-carboxylic acid (34.8 mg, 0.123 mmol) in DMF (0.700 mL) at 0° C. The mixture was stirred at 0° C. for 5 min. 1,2-Phenylenediamine (14 mg, 0.129 mmol) was added. The mixture was warmed to 23° C. and stirred for 5.5 h. Water (10 mL) was added. The mixture was filtered, and the solid was washed with water. The solid (m/z (ES⁺), [M+H]⁺ 373.3. HPLC (A05) t_{R}=2.25 min) was dissolved in AcOH (0.700 mL), and the mixture was heated at 100° C. for 10 min. Water (10 mL) was added. The mixture was filtered, and the solid was washed with sat. NaHCO₃ (10 mL) and water (10 mL). The solid was purified by Waters HPLC (BEH C-18 column, 30×150 mm, 5 micron) eluting with mixtures of water (0.1% TFA) and MeCN (25-45%). The HPLC solution was neutralized with NaHCO₃, and the mixture was partially concentrated. The aq. phase was extracted with EtOAc (2×20 mL). The combined organic phases were concentrated, and the residue was dried under high vacuum to provide the title compound as a solid (6.17 mg, 14%). ¹H NMR (500 MHz, DMSO) δ 7.54 (d, J=2.2 Hz, 1H), 7.51-7.45 (m, 2H), 7.43 (dd, J=8.5, 2.1 Hz, 1H), 7.17 (d, J=8.5 Hz, 1H), 7.06-6.97 (m, 2H), 6.20 (s, 1H), 5.13 (s, 2H), 3.87 (s, 3H). m/z (ES⁺), [M+H]⁺ 355.3. HPLC (A05) t_{R}=2.33 min.

### EXAMPLE B38

### Preparation of Compound A-231

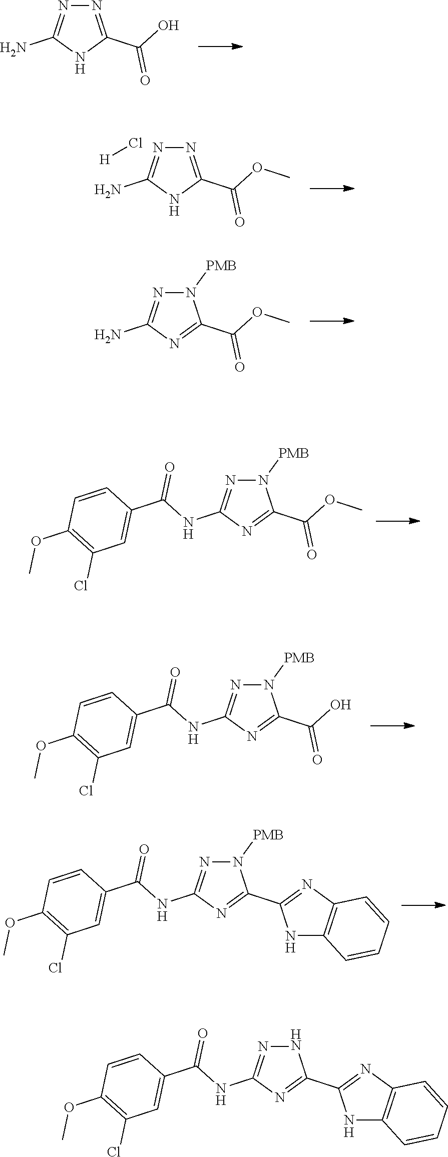

### Step 1: methyl 5-amino-4H-1,2,4-triazole-3-carboxylate hydrochloride

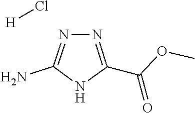

SOCl₂ (14.5 mL, 200 mmol) was added dropwise at 0° C. to a suspension of 5-amino-4H-1,2,4-triazole-3-carboxylic acid (10.0 g, 78.1 mmol) in MeOH (325 mL). After stirring at 0° C. for 30 min, the mixture was refluxed for 2 h. The mixture was concentrated. The residue was diluted in hexanes (250 mL) and stirred at 23° C. for 15 min. The mixture was filtered, and the solid was dried under high vacuum to provide the title compound as a solid (12.3 g, 88%). ¹H NMR (500 MHz, DMSO) δ 7.94 (br, 4H), 3.85 (s, 3H). m/z (ES+), [M−Cl]⁺ 143.1. HPLC (A05) t_{R}=0.41 min.

### Step 2: methyl 5-amino-1-, methyl 5-amino-2-, and methyl 5-amino-4-[(4-methoxyphenyl)methyl]-1,2,4-triazole-3-carboxylate

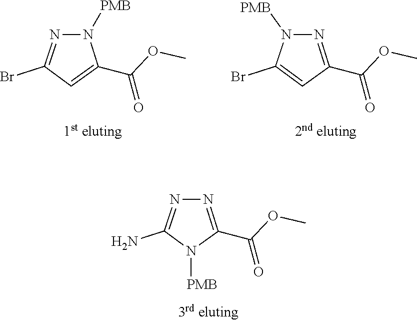

DMF (30 mL) was added to a mixture of methyl 5-amino-4H-1,2,4-triazole-3-carboxylate hydrochloride (2.00 g, 11.2 mmol) and Cs₂CO₃ (10.9 g, 33.6 mmol). After stirring at 23° C. for 30 min, 4-methoxybenzyl chloride (1.41 mL, 12.3 mmol) was added dropwise. The mixture was stirred at 40° C. for 18 h. Water (250 mL) was added, and the aq. phase was extracted with EtOAc (3×100 mL). The combined organic phases were washed with brine (100 mL), dried (MgSO₄), filtered, and concentrated. The residue was purified by silica gel chromatography (80 g cartridge) eluting with DCM and MeOH (0-10%) to provide three compounds: 1^{st} eluting compound: methyl 5-amino-2-[(4-methoxyphenyl)methyl]-1,2,4-triazole-3-carboxylate as a solid (0.334 g, 11%). ¹H NMR (500 MHz, DMSO) δ 7.31-7.13 (m, 2H), 6.97-6.82 (m, 2H), 5.63 (s, 2H), 5.43 (s, 2H), 3.86 (s, 3H), 3.73 (s, 3H). m/z (ES⁺), [M+H]⁺ 263.2. HPLC (A05) t_{R}=1.92 min. 2^{nd} eluting compound: methyl 5-amino-1-[(4-methoxyphenyl)methyl]-1,2,4-triazole-3-carboxylate as a solid (0.801 g, 27%). ¹H NMR (500 MHz, DMSO) δ 7.24-7.14 (m, 2H), 6.97-6.82 (m, 2H), 6.63 (s, 2H), 5.11 (s, 2H), 3.75 (s, 3H), 3.74 (s, 3H). m/z (ES⁺), [M+H]⁺ 263.2. HPLC (A05) t_{R}=1.83 min. 3^{rd} eluting compound: methyl 5-amino-4-[(4-methoxyphenyl)methyl]-1,2,4-triazole-3-carboxylate as a solid (21.9 mg, 1%). ¹H NMR (500 MHz, DMSO) δ 7.16-7.03 (m, 2H), 6.95-6.82 (m, 2H), 6.57 (s, 2H), 5.29 (s, 2H), 3.80 (s, 3H), 3.72 (s, 3H). m/z (ES⁺), [M+H]⁺ 263.2. HPLC (A05) t_{R}=1.82 min.

### Step 3: methyl 5-[(3-chloro-4-methoxy-benzoyl)amino]-2-[(4-methoxyphenyl)-methyl]-1,2,4-triazole-3-carboxylate

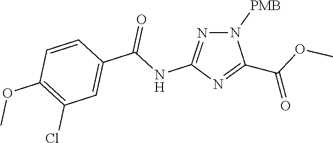

Oxalyl chloride (113 μL, 1.32 mmol) and DMF (7.74 μL, 0.100 mmol) were added at 23° C. to a suspension of 3-chloro-4-methoxy-benzoic acid (205 mg, 1.10 mmol) in DCM (5 mL). The mixture was stirred at 23° C. for 1 h and concentrated. The residue was dissolved in THF (5 mL) and cooled to 0° C. Pyridine (0.242 mL, 3.00 mmol) was added dropwise, and the mixture was stirred at 0° C. for 10 min. Methyl 5-amino-2-[(4-methoxyphenyl)methyl]-1,2,4-triazole-3-carboxylate (262 mg, 1.00 mmol) was added. The mixture was warmed to 23° C. and stirred for 45 min. The mixture was concentrated. Brine (20 mL) was added, and the aq. phase was extracted with EtOAc (3×20 mL). The combined organic phases were washed with brine (20 mL), dried (MgSO₄), filtered, and concentrated. The residue was purified by silica gel chromatography (25 g cartridge) eluting with hexanes and EtOAc (0-100%) to provide the title compound as a solid (253 mg, 59%). ¹H NMR (500 MHz, DMSO) δ 10.98 (s, 1H), 8.08 (d, J=2.2 Hz, 1H), 7.99 (dd, J=8.7, 2.2 Hz, 1H), 7.34-7.24 (m, 3H), 7.01-6.86 (m, 2H), 5.65 (s, 2H), 3.95 (s, 3H), 3.94 (s, 3H), 3.74 (s, 3H). m/z (ES⁺), [M+H]⁺ 431.4. HPLC (A05) t_{R}=2.34 min.

### Step 4: 5-[(3-chloro-4-methoxy-benzoyl)amino]-2-[(4-methoxyphenyl)methyl]-1,2,4-triazole-3-carboxylic acid

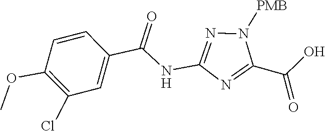

LiOH—H₂O (29.2 mg, 0.696 mmol) was added to a mixture of methyl 5-[(3-chloro-4-methoxy-benzoyl)amino]-2-[(4-methoxyphenyl)methyl]-1,2,4-triazole-3-carboxylate (100 mg, 0.232 mmol) in MeOH (750 THF (750 and water (200 The mixture was stirred at 70° C. for 2 h and concentrated. Water (10 mL) was added. The aq. phase was acidified to pH 3 with 1M aq. HCl. The mixture was filtered, and the solid was dried in a vacuum oven at 40° C. for 18 h to provide the title compound as a solid (69.8 mg, 72%). ¹H NMR (500 MHz, DMSO) δ 10.64 (s, 1H), 8.55 (s, 1H), 8.05 (d, J=2.2 Hz, 1H), 7.36-7.19 (m, 3H), 6.94 (d, J=8.7 Hz, 2H), 5.29 (s, 2H), 3.94 (s, 3H), 3.75 (s, 3H). m/z (ES⁺), [M+H]⁺ 417.4. HPLC (A05) t_{R}=1.95 min.

Keep the lithium carboxylate salt reduces decarboxylation upon acidification.

### Step 5: N-[5-(1H-benzimidazol-2-yl)-1-[(4-methoxyphenyl)methyl]-1,2,4-triazol-3-yl]-3-chloro-4-methoxy-benzamide

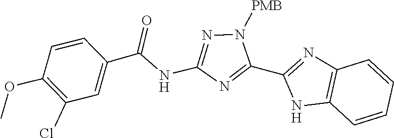

DIEA (99.2 μL, 0.579 mmol) was added to a solution of HATU (69.2 mg, 0.182 mmol) and 5-[(3-chloro-4-methoxy-benzoyl)amino]-2-[(4-methoxyphenyl)methyl]-1,2,4-triazole-3-carboxylic acid (69 mg, 0.166 mmol) in DMF (1 mL) at 0° C. The mixture was stirred at 0° C. for 15 min. o-Phenylenediamine (18.8 mg, 0.174 mmol) was added. The mixture was warmed to 23° C. and stirred for 3 h. Brine (20 mL) was added, and the aq. phase was extracted with EtOAc (3×20 mL). The combined organic phases were washed with brine (20 mL), dried (MgSO₄), filtered, and concentrated. The residue (m/z (ES⁺), [M+H]⁺ 507.4. HPLC (A05) t_{R}=2.45 min) was dissolved in AcOH (1 mL) and stirred at 70° C. for 1 h. The mixture was diluted with EtOAc (10 mL). Sat. NaHCO₃ (10 mL) was added, and the aq. phase was extracted with EtOAc (2×10 mL). The combined organic phases were washed with brine (10 mL), dried (MgSO₄), filtered, and concentrated. The residue was purified by silica gel chromatography (16 g cartridge) eluting with hexanes and EtOAc (0-100%) to provide the title compound as a solid (14.8 mg, 18%). ¹H NMR (400 MHz, DMSO) δ 13.58-13.42 (m, 1H), 10.83 (s, 1H), 8.09 (d, J=2.2 Hz, 1H), 8.01 (dd, J=8.6, 2.2 Hz, 1H), 7.85-7.79 (m, 1H), 7.64-7.55 (m, 1H), 7.44-7.26 (m, 5H), 6.92 (d, J=8.8 Hz, 2H), 6.10 (s, 2H), 3.96 (s, 3H), 3.72 (s, 3H). m/z (ES⁺), [M+H]⁺ 489.4. HPLC (A05) t_{R}=2.58 min.

### Step 6: N-[5-(1H-benzimidazol-2-yl)-1H-1,2,4-triazol-3-yl]-3-chloro-4-methoxy-benzamide

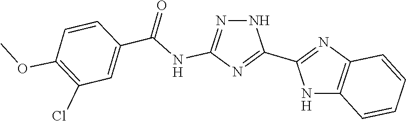 [SMILES: COc1ccc(C(=O)Nc2n[nH]c(-c3nc4ccccc4[nH]3)n2)cc1Cl]

TFA (1 mL) was added to N-[5-(1H-benzimidazol-2-yl)-1-[(4-methoxyphenyl)methyl]-1,2,4-triazol-3-yl]-3-chloro-4-methoxy-benzamide (14 mg, 0.0286 mmol), and the mixture was stirred at 23° C. for 30 min. The mixture was concentrated. The residue was dissolved in DMSO (0.500 mL). NEt₃ (0.500 mL) was added, and the mixture was stirred at 23° C. for 5 min. Water (10 mL) was added, and the aq. phase was extracted with EtOAc (3×10 mL). The combined organic phases were washed with brine (10 mL), dried (MgSO₄), filtered, and concentrated. The residue was purified by silica gel chromatography (7 g cartridge) eluting with DCM and MeOH (0-5%) to provide the title compound as a solid (4.3 mg, 41%). ¹H NMR (500 MHz, DMSO) δ 12.96 (s, 1H), 8.22 (s, 1H), 8.18-8.01 (m, 1H), 7.74-7.60 (m, 1H), 7.59-7.45 (m, 1H), 7.38-7.29 (m, 1H), 7.28-7.14 (m, 2H), 3.97 (s, 3H). m/z (ES⁺), [M+H]⁺ 369.3. HPLC (A05) t_{R}=2.15 min.

### EXAMPLE B39

### Preparation of Compound A-92

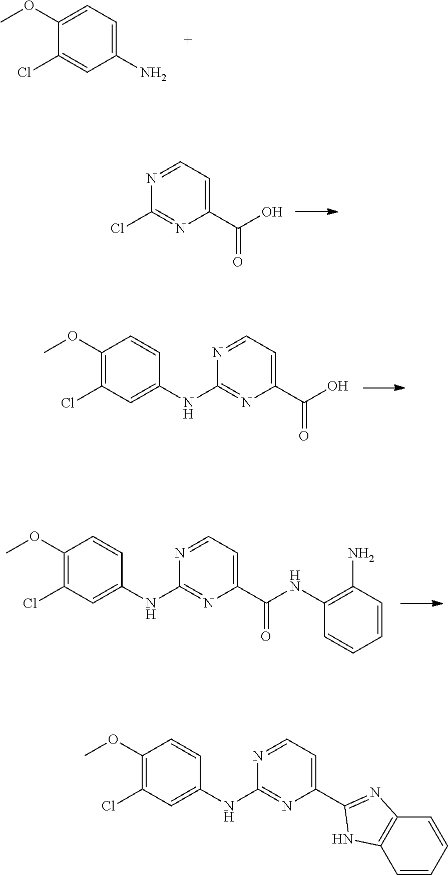

### Step 1: 2-(3-chloro-4-methoxy-anilino)pyrimidine-4-carboxylic acid

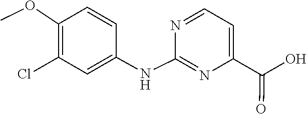 [SMILES: COc1ccc(Nc2nccc(C(=O)O)n2)cc1Cl]

2-Chloropyrimidine-4-carboxylic acid (317 mg, 2.00 mmol) and 3-chloro-4-methoxy-aniline (394 mg, 2.50 mmol) were dissolved in DMSO (6 mL). The mixture was stirred at 100° C. for 2 h. The mixture was diluted with EtOAc (50 mL). The product was extracted with 1N NaOH (3×25 mL). The aq. phase was washed with EtOAc (2×50 mL) and acidified to pH 4 with conc. HCl. The mixture was filtered, and the solid was dried in a vacuum oven at 45° C. for 18 h to provide the title compound as a solid (250 mg, 45%). ¹H NMR (500 MHz, DMSO) δ 13.67 (br, 1H), 9.93 (s, 1H), 8.68 (d, J=4.9 Hz, 1H), 7.99 (d, J=2.6 Hz, 1H), 7.70 (dd, J=8.9, 2.6 Hz, 1H), 7.28 (d, J=4.9 Hz, 1H), 7.12 (d, J=9.1 Hz, 1H), 3.82 (s, 3H). m/z (ES⁺), [M+H]⁺ 280.1. HPLC (A05) t_{R}=1.83 min.

### Step 2: N-(2-aminophenyl)-2-(3-chloro-4-methoxy-anilino)pyrimidine-4-carboxamide

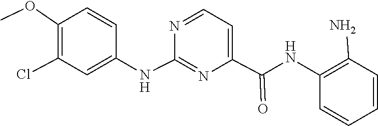 [SMILES: COc1ccc(Nc2nccc(C(=O)Nc3ccccc3N)n2)cc1Cl]

DIEA (0.271 mL, 1.56 mmol) was added to a solution of HATU (217 mg, 0.570 mmol) and 2-(3-chloro-4-methoxy-anilino)pyrimidine-4-carboxylic acid (145 mg, 0.518 mmol) in DMF (2 mL) at 0° C. The mixture was stirred at 0° C. for 5 min. o-Phenylenediamine (58.9 mg, 0.544 mmol) was added. The mixture was warmed to 23° C. and stirred for 2.5 h. Water (20 mL) was added, and the mixture was stirred at 23° C. for 5 min. The mixture was filtered, and the solid was washed with water (20 mL) and purified by preparative HPLC (BEH C18 30×150) eluting with water (10 mM (NH₄)(HCO₂)) and MeCN (50-60%). The residue was diluted in sat. NaHCO₃ (20 mL), and the mixture was stirred at 23° C. for 5 min. The mixture was filtered, and the solid was washed with water (20 mL) and dried in a vacuum over at 45° C. for 18 h to provide the title compound as a solid (52.4 mg, 27%). ¹H NMR (500 MHz, DMSO) δ 9.88 (s, 1H), 9.64 (s, 1H), 8.75 (d, J=4.9 Hz, 1H), 7.83 (s, 1H), 7.77 (d, J=8.7 Hz, 1H), 7.61 (d, J=7.6 Hz, 1H), 7.39 (d, J=4.9 Hz, 1H), 7.12 (d, J=9.0 Hz, 1H), 6.98 (t, J=7.1 Hz, 1H), 6.86 (d, J=7.8 Hz, 1H), 6.68 (t, J=7.6 Hz, 1H), 5.01 (s, 2H), 3.84 (s, 3H). m/z (ES⁺), [M+H]⁺ 370.3. HPLC (A05) t_{R}=2.40 min.

### Step 3: 4-(1H-benzimidazol-2-yl)-N-(3-chloro-4-methoxy-phenyl)pyrimidin-2-amine

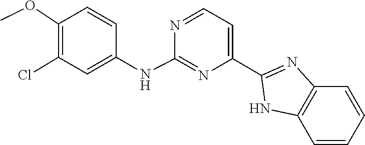 [SMILES: COc1ccc(Nc2nccc(-c3nc4ccccc4[nH]3)n2)cc1Cl]

AcOH (0.500 mL) was added to N-(2-aminophenyl)-2-(3-chloro-4-methoxy-anilino)pyrimidine-4-carboxamide (31.3 mg, 0.0846 mmol), and the mixture was stirred at 70° C. for 6 h. The mixture was diluted with EtOAc (10 mL). Sat. NaHCO₃ (10 mL) was added, and the aq. phase was extracted with EtOAc (2×10 mL). The combined organic phases were washed with brine (10 mL), dried (MgSO₄), filtered, and concentrated. The residue was purified by preparative HPLC (BEH C18 30×150) eluting with water (10 mM (NH₄)(HCO₂)) and MeCN (44-64%) to provide the title compound as a solid (8.2 mg, 28%). ¹H NMR (500 MHz, DMSO) δ 12.89 (s, 1H), 9.66 (s, 1H), 8.65 (d, J=4.9 Hz, 1H), 7.96-7.89 (m, 1H), 7.85-7.81 (m, J=2.4 Hz, 1H), 7.79-7.72 (m, 1H), 7.71-7.64 (m, 1H), 7.63 (d, J=5.0 Hz, 1H), 7.37-7.24 (m, 2H), 7.16 (d, J=9.0 Hz, 1H), 3.86 (s, 3H). m/z (ES+), [M+H]⁺ 352.3. HPLC (A05) t_{R}=2.41 min.

### EXAMPLE B40

### Preparation of Compound A-194

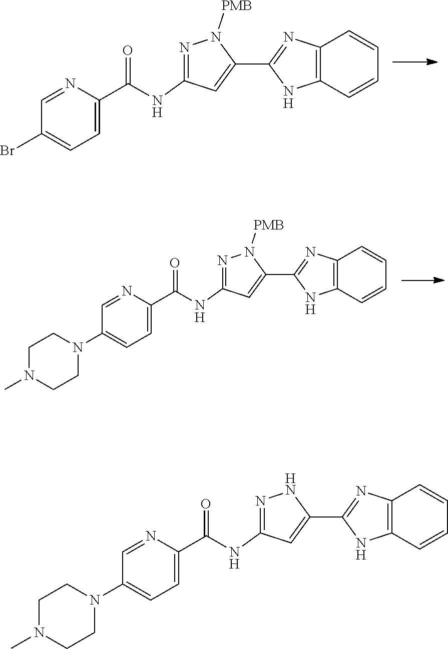

### Step 1: N-[5-(1H-benzimidazol-2-yl)-1-[(4-methoxyphenyl)methyl]pyrazol-3-yl]-5-(4-methylpiperazin-1-yl)pyridine-2-carboxamide

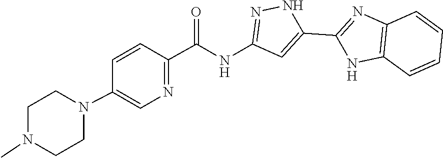 [SMILES: CN1CCN(c2ccc(C(=O)Nc3cc(-c4nc5ccccc5[nH]4)[nH]n3)nc2)CC1]

1-Methylpiperazine (35.8 mg, 0.358 mmol) was added to a solution of N-[5-(1H-benzimidazol-2-yl)-1-[(4-methoxyphenyl)methyl]pyrazol-3-yl]-5-bromo-pyridine-2-carboxamide (L-25) (60 mg, 0.119 mmol) and DIEA (0.0816 mL, 0.477 mmol) in DMSO (1 mL). The mixture was heated at 100° C. for 96 h. Water (20 mL) was added, and the mixture was filtered. The solid was purified by silica gel chromatography (12 g cartridge) eluting with DCM and MeOH (0-10%) to provide the title compound as a solid (38 mg, 61%). ¹H NMR (400 MHz, DMSO) δ 13.14 (s, 1H), 10.24 (s, 1H), 8.37 (d, J=2.6 Hz, 1H), 7.97 (d, J=8.9 Hz, 1H), 7.73 (d, J=9.9 Hz, 1H), 7.54 (d, J=11.5 Hz, 2H), 7.47 (dd, J=8.9, 3.0 Hz, 1H), 7.32-7.19 (m, 4H), 6.87-6.82 (m, 2H), 6.01 (s, 2H), 3.68 (s, 3H), 3.41-3.36 (m, 4H), 2.48-2.44 (m, 4H), 2.23 (s, 3H). m/z (ES⁺), [M+H]⁺ 523.2. HPLC (A05) t_{R}=2.07 min.

### Step 2: N-[5-(1H-benzimidazol-2-yl)-1H-pyrazol-3-yl]-5-(4-methylpiperazin-1-yl)pyridine-2-carboxamide

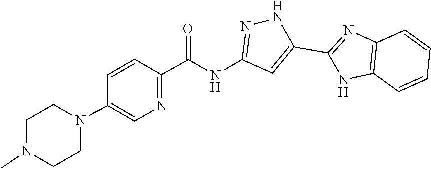 [SMILES: CN1CCN(c2ccc(C(=O)Nc3cc(-c4nc5ccccc5[nH]4)[nH]n3)nc2)CC1]

TFA (1 mL) was added to N-[5-(1H-benzimidazol-2-yl)-1-[(4-methoxyphenyl)methyl]pyrazol-3-yl]-5-(4-methylpiperazin-1-yl)pyridine-2-carboxamide (38 mg, 0.0727 mmol), and the mixture was stirred at 23° C. for 1 h. The mixture was concentrated. The residue was dissolved in DMSO (0.500 mL). NEt₃ (0.500 mL) was added, and the mixture was stirred at 23° C. for 30 min. Water (10 mL) was added. The mixture was filtered, and the solid was washed with water (10 mL), acetone (10 mL), DCM (10 mL), and acetone (10 mL). The solid was purified by preparative HPLC (BEH C18 30×150) eluting with water (10 mM (NH₄)(HCO₃)) and MeCN (29-39%) to provide the title compound as a solid (12 mg, 41%). ¹H NMR (400 MHz, DMSO) δ 13.55 [13.10] (s, 1H), 12.91 [12.71] (s, 1H), [11.21] 10.23 (s, 1H), 8.40 (d, J=2.8 Hz, 1H), 7.99 (d, J=8.8 Hz, 1H), 7.71-7.58 (m, 1H), 7.59-7.37 [7.00-6.83] (m, 3H), 7.30-7.11 (m, 2H), 3.42-3.37 (m, 4H), 2.49-2.44 (m, J=7.4, 3.1 Hz, 4H), 2.24 (s, 3H). m/z (ES⁺), [M+H]⁺ 403.1. HPLC (A05) t_{R}=1.78 min.

### EXAMPLE B41

### Preparation of Compound A-195

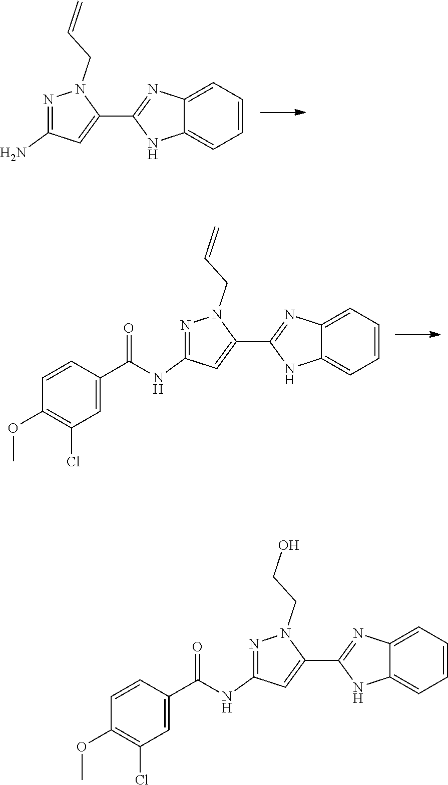 [SMILES: C=CCn1nc(N)cc1-c1nc2ccccc2[nH]1 ; C=CCn1nc(NC(=O)c2ccc(OC)c(Cl)c2)cc1-c1nc2ccccc2[nH]1 ; COc1ccc(C(=O)Nc2cc(-c3nc4ccccc4[nH]3)n(CCO)n2)cc1Cl]

### Step 1: N-[1-allyl-5-(1H-benzimidazol-2-yl)pyrazol-3-yl]-3-chloro-4-methoxy-benzamide

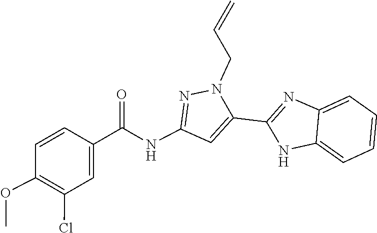 [SMILES: C=CCn1nc(NC(=O)c2ccc(OC)c(Cl)c2)cc1-c1nc2ccccc2[nH]1]

DIEA (0.0735 mL, 0.922 mmol) was added to a solution of 3-chloro-4-methoxy-benzoic acid (54 mg, 0.290 mmol) and HATU (110 mg, 0.290 mmol) in DMF (0.500 mL). A solution of 1-allyl-5-(1H-benzimidazol-2-yl)pyrazol-3-amine (J-16) (63 mg, 0.263 mmol) in DMF (1.50 mL) was added. The mixture was warmed to 23° C. and stirred for 18 h. Water (10 mL) was added, and the aq. phase was extracted with EtOAc (3×5 mL). The combined organic phases were washed with brine (10 mL), dried (MgSO₄), filtered, and concentrated. The residue was purified by silica gel chromatography eluting with hexanes and EtOAc (30-60%) to provide the title compound as a solid (85 mg, 79%). ¹H NMR (400 MHz, DMSO) δ 13.10 (s, 1H), 11.12 (s, 1H), 8.17 (d, J=2.2 Hz, 1H), 8.09 (dd, J=8.7, 2.2 Hz, 1H), 7.71 (d, J=7.4 Hz, 1H), 7.53 (d, J=8.3 Hz, 2H), 7.30-7.20 (m, 3H), 6.08 (ddd, J=15.8, 10.4, 5.4 Hz, 1H), 5.49 (d, J=5.5 Hz, 2H), 5.11 (ddd, J=18.7, 13.7, 1.6 Hz, 2H), 3.95 (s, 3H). m/z (ES⁺), [M−H]⁻ 406.4. HPLC (A05) t_{R}=2.48 min.

### Step 2: N-[5-(1H-benzimidazol-2-yl)-1-(2-hydroxyethyl)pyrazol-3-yl]-3-chloro-4-methoxy-benzamide

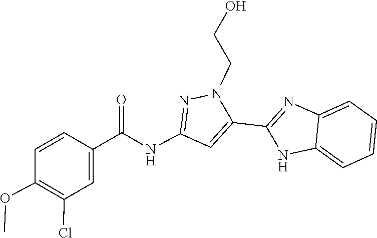 [SMILES: COc1ccc(C(=O)Nc2cc(-c3nc4ccccc4[nH]3)n(CCO)n2)cc1Cl]

A mixture of N-[1-allyl-5-(1H-benzimidazol-2-yl)pyrazol-3-yl]-3-chloro-4-methoxy-benzamide (17 mg, 0.0417 mmol) in 1,4-dioxane (1.20 mL) and water (0.400 mL) was cooled to 0° C., and 2,6-lutidine (9.66 μL, 0.0834 mmol), potassium osmate(IV) dihydrate (1.00 mg, 0.00271 mmol), and NaIO₄ (17.8 mg, 0.0834 mmol) were added. The mixture was warmed to 23° C. and stirred for 23 h. Water (20 mL) was added, and the aq. phase was extracted with EtOAc (3×25 mL). The combined organic phases were washed with brine (40 mL), dried (MgSO₄), filtered, and concentrated. The residue (m/z (ES⁺), [M−H]⁻ 408.5. HPLC (A05) t_{R}=2.17 min) was suspended in MeOH (1 mL), and NaBH₄ (2.37 mg, 0.0625 mmol) was added. The mixture was stirred at 40° C. for 1 h. After cooling to 23° C., water (10 mL) was added. The aq. phase was extracted with EtOAc (3×10 mL). The combined organic phases were washed with brine (50 mL), dried (MgSO₄), filtered, and concentrated. The residue was purified by preparative HPLC (BEH C18 30×150) eluting with water (NH₄)(HCO₃)) and MeCN (35-55%) to provide the title compound as a solid (9.00 mg, 52%). ¹H NMR (400 MHz, DMSO) δ 11.06 (s, 1H), 8.17 (d, J=2.2 Hz, 1H), 8.09 (dd, J=8.7, 2.3 Hz, 1H), 7.61 (s, 2H), 7.46 (s, 1H), 7.28 (d, J=8.9 Hz, 1H), 7.24 (dd, J=6.0, 3.0 Hz, 2H), 4.86 (t, J=6.0 Hz, 2H), 3.95 (s, 3H), 3.86 (t, J=6.2 Hz, 2H). m/z (ES⁺), [M−H]⁻ 410.5. HPLC (A05) t_{R}=2.22 min.

### EXAMPLE B42

### Preparation of Compound A-228

### Step 1: N-[5-(1H-benzimidazol-2-yl)-1-methyl-pyrazol-3-yl]-6-(6-oxo-1,3,4,7,8,8a-hexahydropyrrolo[1,2-a]pyrazin-2-yl)pyridine-3-carboxamide

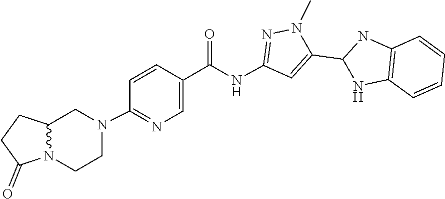 [SMILES: Cn1nc(NC(=O)c2ccc(N3CCN4C(=O)CCC4C3)nc2)cc1C1Nc2ccccc2N1]

DIEA (0.0494 mL, 0.283 mmol) was added to a solution of (8aS)-2,3,4,7,8,8a-hexahydro-1H-pyrrolo[1,2-a]pyrazin-6-one; hydrochloride (40.1 mg, 0.227 mmol) and N-[5-(1H-benzimidazol-2-yl)-1-methyl-pyrazol-3-yl]-6-chloro-pyridine-3-carboxamide (50 mg, 0.142 mmol) in DMF (1 mL). The mixture was heated at 100° C. for one week. The mixture was cooled to 23° C. and water (30 mL) was added. The aq. phase was extracted with EtOAc (3×15 mL), and the combined organic phases were washed with brine (45 mL), dried (MgSO₄), filtered, and concentrated. The residue was purified by silica gel chromatography (12 g cartridge) eluting with DCM and MeCN (0-100%) to provide a solid. The solid was suspended in DMSO (0.500 mL). Water (10 mL) was added, and the aq. phase was extracted with EtOAc (3×15 mL). The combined organic phases were washed with sat. aq. NaHCO₃ (30 mL), and concentrated to provide the title compound as a solid (45.4 mg, 70%). ¹H NMR (400 MHz, DMSO-d₆) δ 10.71 (s, 1H), 8.81 (d, J=2.4 Hz, 1H), 8.22-8.14 (m, 1H), 7.50 (s, 2H), 7.28 (s, 1H), 7.03 (s, 2H), 6.98 (d, J=9.1 Hz, 1H), 4.64 (s, 1H), 4.51 (s, 1H), 4.31 (s, 3H), 3.89 (s, 1H), 3.59 (s, 1H), 2.83 (d, J=7.3 Hz, 2H), 2.73-2.64 (m, 1H), 2.27 (d, J=10.3 Hz, 2H), 2.20-2.11 (m, 1H), 1.65 (s, 1H). m/z (ES⁺), [M+H]⁺ 457.4 HPLC (A05) t_{R}=2.05 min.

### EXAMPLE B43

### Preparation of Compound A-229

### Step 1: N-[5-(1H-benzimidazol-2-yl)-1-methyl-pyrazol-3-yl]-6-[(3R)-3-methylmorpholin-4-yl]pyridine-3-carboxamide

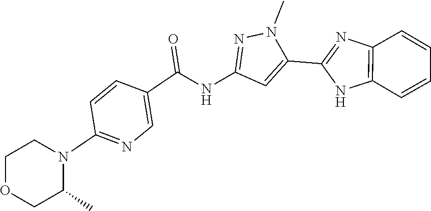 [SMILES: C[C@@H]1COCCN1c1ccc(C(=O)Nc2cc(-c3nc4ccccc4[nH]3)n(C)n2)cn1]

(3R)-3-methylmorpholine (68.8 mg, 0.680 mmol) was added to a solution of N-[5-(1H-benzimidazol-2-yl)-1-methyl-pyrazol-3-yl]-6-chloro-pyridine-3-carboxamide (80 mg, 0.227 mmol) and DIEA (0.0776 mL, 0.454 mmol) in DMSO (1.50 mL). The mixture was heated to 140° C. and stirred for 72 h. EtOAc (60 mL) was added, and the organic phase was washed with water (3×40 mL), brine (50 mL), dried (MgSO₄), filtered, and concentrated. The residue was purified by silica gel chromatography (12 g cartridge) eluting with DCM and MeOH (0-10%) followed by a preparative HPLC purification (BEH C18 30×150) eluting with water (10 mM (NH₄)(HCO₂)) and MeCN (30-40%) to provide the title compound as a solid (43 mg, 45%). ¹H NMR (400 MHz, DMSO-d6) δ 12.96 (s, 1H), 10.79 (s, 1H), 8.77 (d, J=2.4 Hz, 1H), 8.12 (dd, J=9.0, 2.5 Hz, 1H), 7.67 (d, J=7.8 Hz, 1H), 7.48 (d, J=7.7 Hz, 1H), 7.42 (s, 1H), 7.25-7.15 (m, 2H), 6.79 (d, J=9.1 Hz, 1H), 4.40 (qd, J=7.0, 2.5 Hz, 1H), 4.25 (s, 3H), 3.98 (dd, J=13.2, 2.4 Hz, 1H), 3.90 (dd, J=11.3, 3.5 Hz, 1H), 3.69 (d, J=11.3 Hz, 1H), 3.58 (dd, J=11.5, 3.1 Hz, 1H), 3.43 (td, J=11.6, 3.0 Hz, 1H), 3.08 (td, J=12.9, 3.8 Hz, 1H), 1.12 (d, J=6.7 Hz, 3H). m/z (ES⁺), [M+H]⁺ 418.4. HPLC (A05) t_{R}=2.17 min.

### EXAMPLE B44

### Preparation of Compound A-230

### Step 1: N-[5-(1H-benzimidazol-2-yl)-1-methyl-pyrazol-3-yl] -6-[(3S)-3-methylmorpholin-4-yl]pyridine-3-carboxamide

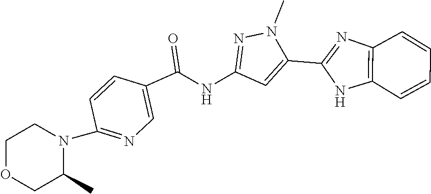 [SMILES: C[C@H]1COCCN1c1ccc(C(=O)Nc2cc(-c3nc4ccccc4[nH]3)n(C)n2)cn1]

(3S)-3-methylmorpholine (68.8 mg, 0.680 mmol) was added to a solution of N-[5-(1H-benzimidazol-2-yl)-1-methyl-pyrazol-3-yl]-6-chloro-pyridine-3-carboxamide (80 mg, 0.227 mmol) and DIEA (0.0776 mL, 0.454 mmol) in DMSO (1.50 mL). The mixture was heated to 140° C. and stirred for 72 h. EtOAc (60 mL) was added, and the organic phase was washed with water (3×40 mL), brine (50 mL), dried (MgSO₄), filtered, and concentrated. The residue was purified by silica gel chromatography (12 g cartridge) eluting with DCM and MeOH (0-5%) followed by a preparative HPLC purification (BEH C18 30 ×150) eluting with water (10 mM (NH₄)(HCO₂)) and MeCN (33-43%) to provide the title compound as a solid (37 mg, 39%). ¹H NMR (400 MHz, DMSO-d6) δ 13.00 (s, 1H), 10.79 (s, 1H), 8.77 (d, J=2.5 Hz, 1H), 8.12 (dd, J=9.0, 2.5 Hz, 1H), 7.72-7.57 (m, 1H), 7.57-7.45 (m, 1H), 7.42 (s, 1H), 7.24-7.15 (m, 2H), 6.79 (d, J=9.1 Hz, 1H), 4.44-4.36 (m, 1H), 4.25 (s, 3H), 3.98 (dd, J=13.4, 2.2 Hz, 1H), 3.90 (dd, J=11.3, 3.7 Hz, 1H), 3.69 (d, J=11.3 Hz, 1H), 3.58 (dd, J=11.4, 3.0 Hz, 1H), 3.43 (td, J=11.7, 3.1 Hz, 1H), 3.08 (td, J=12.9, 3.8 Hz, 1H), 1.12 (d, J=6.7 Hz, 3H). m/z (ES⁺), [M+H]⁺ 418.4. HPLC (A05) t_{R}=2.17 min.

### EXAMPLE B45

### Preparation of Compound A-231

### Step 1: 6-(4-acetylpiperazin-1-yl)-N-[5-(1H-benzimidazol-2-yl)-1-methyl-pyrazol-3-yl]pyridine-3-carboxamide

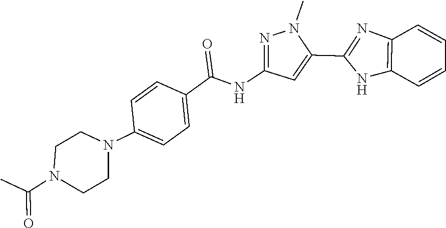 [SMILES: CC(=O)N1CCN(c2ccc(C(=O)Nc3cc(-c4nc5ccccc5[nH]4)n(C)n3)cc2)CC1]

1-piperazin-1-ylethanone (29.1 mg, 0.227 mmol) was added to a solution of N-[5-(1H-benzimidazol-2-yl)-1-methyl-pyrazol-3-yl]-6-chloro-pyridine-3-carboxamide (80 mg, 0.227 mmol) in DMSO (1.50 mL). The mixture was heated to 110° C. and stirred for 18 h. EtOAc (60 mL) was added, and the organic phase was washed with water (3×40 mL), brine (50 mL), dried (MgSO₄), filtered, and concentrated. The residue was purified by silica gel chromatography (12 g cartridge) eluting with DCM and MeOH (0-10%) to provide the title compound as a solid (43.5 mg, 43%). ¹H NMR (400 MHz, DMSO-d6) δ 13.06 (s, 1H), 10.86 (s, 1H), 8.82 (d, J=2.5 Hz, 1H), 8.18 (dd, J=9.1, 2.5 Hz, 1H), 7.75-7.63 (m, 1H), 7.60-7.49 (m, 1H), 7.47 (s, 1H), 7.30-7.21 (m, 2H), 6.91 (d, J=9.1 Hz, 1H), 4.30 (s, 3H), 3.71 (dd, J=6.9, 3.6 Hz, 2H), 3.63 (dd, J=6.6, 3.4 Hz, 2H), 3.56 (dd, J=6.6, 3.8 Hz, 4H), 2.06 (s, 3H). m/z (ES⁺), [M+H]⁺ 445.4. HPLC (A05) t_{R}=2.02 min.

### EXAMPLE B46

### Preparation of Compound A-232

### Step 1: N-[5-(1H-benzimidazol-2-yl)-1-methyl-pyrazol-3-yl]-6-chloro-4-methyl-pyridine-3-carboxamide

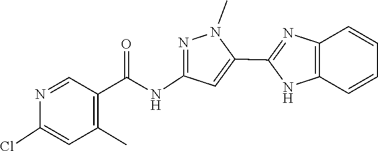 [SMILES: Cc1cc(Cl)ncc1C(=O)Nc1cc(-c2nc3ccccc3[nH]2)n(C)n1]

6-chloro-4-methyl-pyridine-3-carboxylic acid (80.5 mg, 0.469 mmol) and HATU (178 mg, 0.469 mmol) were dissolved in DMF (3 mL) at 0° C. DIEA (0.161 mL, 0.938 mmol) was added, and the mixture was stirred at 0° C. for 5 min. 5-(1H-benzimidazol-2-yl)-1-methyl-pyrazol-3-amine (100 mg, 0.469 mmol) was added. The mixture was warmed to 23° C. and stirred for 18 h. The mixture was concentrated, and the residue was purified by silica gel chromatography (12 g cartridge) eluting with DCM and MeOH (0-5%) to provide the title compound as a solid (158 mg, 92%). ¹H NMR (400 MHz, DMSO-d6) δ 13.08 (s, 1H), 11.27 (s, 1H), 8.50 (s, 1H), 7.72 (d, J=7.9 Hz, 1H), 7.58-7.47 (m, 3H), 7.25 (dtd, J=16.5, 7.3, 1.4 Hz, 2H), 4.30 (s, 3H), 2.44 (s, 3H). m/z (ES⁺), [M+H]⁺ 367.3. HPLC (A05) t_{R}=2.19 min.

### Step 2: N-[5-(1H-benzimidazol-2-yl)-1-methyl-pyrazol-3-yl]-4-methyl-6-morpholino-pyridine-3-carboxamide

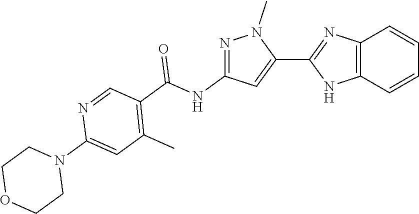 [SMILES: Cc1cc(N2CCOCC2)ncc1C(=O)Nc1cc(-c2nc3ccccc3[nH]2)n(C)n1]

Morpholine (0.0154 mL, 0.177 mmol) was added to a solution of N-[5-(1H-benzimidazol-2-yl)-1-methyl-pyrazol-3-yl]-6-chloro-4-methyl-pyridine-3-carboxamide (65 mg, 0.177 mmol) and DIEA (0.0607 mL, 0.354 mmol) in DMSO (1 mL). The mixture was heated to 120° C. and stirred for 18 h. Water (20 mL) was added, and the precipitate was filtered. The residue was purified by silica gel chromatography (12 g cartridge) eluting with DCM and MeOH (0-5%) to provide the title compound as a solid (42.2 mg, 57%). ¹H NMR (400 MHz, DMSO-d6) δ 13.04 (s, 1H), 10.82 (s, 1H), 8.34 (s, 1H), 7.75-7.66 (m, 1H), 7.54 (dd, J=16.3, 4.3 Hz, 1H), 7.47 (s, 1H), 7.28-7.19 (m, 2H), 6.74 (s, 1H), 4.29 (s, 3H), 3.71-3.68 (m, 4H), 3.56-3.52 (m, 4H), 2.41 (s, 3H). m/z (ES⁺), [M+H]⁺ 418.4. HPLC (A05) t_{R}=2.13 min.

### EXAMPLE B47

### Preparation of Compound A-233

### Step 1: 6-(4-acetylpiperazin-1-yl)-N-[5-(1H-benzimidazol-2-yl)-1-[(4-methoxyphenyl)methyl]pyrazol-3-yl]pyridine-3-carboxamide

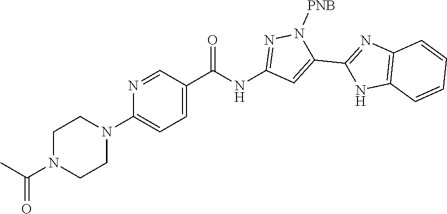

1-piperazin-1-ylethanone (29.1 mg, 0.227 mmol) was added to a solution of N-[5-(1H-benzimidazol-2-yl)-1-[(4-methoxyphenyl)methyl]pyrazol-3-yl]-6-chloro-pyridine-3-carboxamide (104 mg, 0.227 mmol) in DMSO (1.50 mL). The mixture was heated to 110° C. and stirred for 18 h. Water (20 mL) was added, and the precipitate was filtered. The residue was purified by silica gel chromatography (12 g cartridge) eluting with DCM and MeOH (0-10%) to provide the title compound as a solid (87.2 mg, 70%). ¹H NMR (400 MHz, DMSO-d6) δ 13.11 (s, 1H), 10.89 (s, 1H), 8.81 (d, J=2.4 Hz, 1H), 8.17 (dd, J=9.1, 2.4 Hz, 1H), 7.71 (d, J=7.3 Hz, 1H), 7.56-7.50 (m, 2H), 7.29-7.20 (m, 4H), 6.90 (d, J=9.1 Hz, 1H), 6.87-6.83 (m, 2H), 6.03 (s, 2H), 3.72-3.67 (m, 5H), 3.65-3.60 (m, 2H), 3.57-3.52 (m, 4H), 2.05 (s, 3H). m/z (ES⁺), [M+H]⁺ 551.4. HPLC (A05) t_{R}=2.29 min.

### Step 2: 6-(4-acetylpiperazin-1-yl)-N-[5-(1H-benzimidazol-2-yl)-1-[(4-methoxyphenyl)methyl]pyrazol-3-yl]pyridine-3-carboxamide

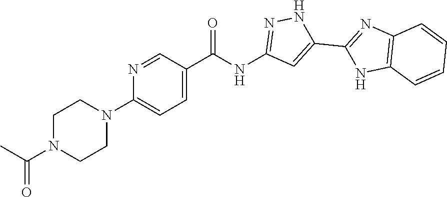 [SMILES: CC(=O)N1CCN(c2ccc(C(=O)Nc3cc(-c4nc5ccccc5[nH]4)[nH]n3)cn2)CC1]

TFA (1.5 mL) was added to 6-(4-acetylpiperazin-1-yl)-N-[5-(1H-benzimidazol-2-yl)-1-[(4-methoxyphenyl)methyl]pyrazol-3-yl]pyridine-3-carboxamide (87 mg, 0.158 mmol), and the mixture was stirred at 23° C. for 1 h. The mixture was concentrated. The residue was dissolved in DMSO (0.6 mL). NEt₃ (0.6 mL) was added, and the mixture was stirred at 23° C. for 30 min. Water (10 mL) was added. The precipitate was filtered, washed with water (10 mL), acetone (10 mL), DCM (10 mL) and acetone (10 mL) to provide the title compound as a solid in a 4:1 mixture of tautomers (38.3 mg, 56%). ¹H NMR (400 MHz, DMSO-d6) δ 13.45 [13.12] (s, 1H), 13.00 [12.71] (s, 1H), [10.94] 10.81 (s, 1H), 8.81 (s, 1H) 8.18 (d, J=6.7 Hz, 1H), 7.71-7.57 (m, 1H), 7.58-7.46 (m, 1H), 7.41 [6.63] (s, 1H), 7.30-7.12 (m, 2H), 7.00-6.84 (m, 1H), 3.76-3.69 (m, 2H), 3.68-3.61 (m, 2H), 3.59-3.53 (m, 4H), 2.06 (s, 3H). m/z (ES⁺), [M+H]⁺ 431.4. HPLC (A05) t_{R}=1.95 min.

### EXAMPLE B48

### Preparation of Compound A-234

### Step 1: N-[bis[(4-methoxyphenyl)methyl]carbamothioyl]-1H-benzimidazole-2-carboxamide

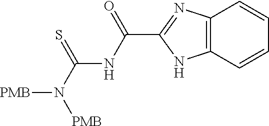

Oxalyl chloride (2.54 mL, 29.6 mmol) was added dropwise at 0° C. to a mixture of 1H-benzimidazole-2-carboxylic acid (3 g, 18.5 mmol) and DMF (0.143 mL, 1.85 mmol) in DCM (180 mL). The mixture was warmed to 23° C., stirred for 2 h, and concentrated under reduced pressure. The resulting solid was suspended in THF (90 mL). NaSCN (2.51 g, 31 mmol) was added via addition funnel as a solution in THF (90 mL) over 30 minutes and the mixture was stirred for 3 days, then filtered through Celite. The filtrate was concentrated under reduced pressure to give a solid. The solid was suspended in MeCN (180 mL). Bis(4-methoxybenzyl)amine (4.76 g, 18.5 mmol) was added to the mixture, followed by NEt₃ (7.74 mL, 55.5 mmol). The mixture was stirred at 23° C. for 20 h and refluxed for 30 min. The reaction was concentrated, and water (100 mL) was added. The aq. phase was extracted with EtOAc (5×20 mL). The combined organic phases were washed with brine (100 mL), dried (MgSO₄), filtered, and concentrated. The residue was purified by silica gel chromatography (25 g cartridge) eluting with hexanes and EtOAc (0-100%) to provide the title compound as a solid (6.62 g, 78%). ¹H NMR (400 MHz, DMSO-d6) δ 7.82-7.50 (m, 2H), 7.37 (d, J=8.2 Hz, 1H), 7.33 (s, 1H), 7.27 (d, J=8.6 Hz, 2H), 7.19 (d, J=8.7 Hz, 1H), 6.91 (dd, J=18.2, 8.8 Hz, 5H), 5.07 (s, 2H), 4.70 (s, 2H), 3.73 (s, 6H). m/z (ES⁺), [M+H]⁺ 461.7. HPLC (A05) t_{R}=2.57 min.

### Step 2: (NZ)-N-[[bis[(4-methoxyphenyl)methyl]amino]-methylsulfanyl-methylene]-1H-benzimidazole-2-carboxamide

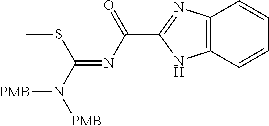

Methyl iodide (0.895 mL, 14.4 mmol) was added to a mixture of N-[bis[(4-methoxyphenyl)methyl]carbamothioyl]-1H-benzimidazole-2-carboxamide (6.62 g, 14.4 mmol) and NaHCO₃ (3.62 g, 43.1 mmol) in THF (145 mL). The mixture was stirred at 70° C. for 18 h and cooled to 23° C. Water (50 mL) was added, and the aq. phase was extracted with EtOAc (5×20 mL). The combined organic phases were washed with brine (50 mL), dried (MgSO₄), filtered, and concentrated. The residue was purified by silica gel chromatography (80 g cartridge) eluting with DCM and MeCN (0-100%) to provide the title compound as a solid (2.25 g, 33%). ¹H NMR (400 MHz, DMSO-d6) δ 12.89 (s, 1H), 7.72 (d, J=8.2 Hz, 1H), 7.49 (d, J=8.7 Hz, 2H), 7.31 (d, J=8.7 Hz, 4H), 7.06 (d, J=8.8 Hz, 1H), 6.94 (d, J=8.4 Hz, 4H), 4.73 (s, 4H), 3.76 (s, 6H), 2.37 (s, 3H). m/z (ES⁺) [M+H]⁺ 477.1, HPLC (A05) t_{R}=2.33 min.

### Step 3: 5-(1H-benzimidazol-2-yl)-1-methyl-1,2,4-triazol-3-amine

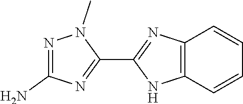 [SMILES: Cn1nc(N)nc1-c1nc2ccccc2[nH]1]

(NZ)-N-[[bis[(4-methoxyphenyl)methyl]amino]-methylsulfanyl-methylene]-1H-benzimidazole-2-carboxamide (2.25 g, 4.27 mmol) was suspended in EtOH (42 mL). Methylhydrazine (1.90 mL, 36.3 mmol) was added, and the mixture was stirred at 90° C. for 4 h, cooled to 23° C., and concentrated. Water (100 mL) was added, and the aq. phase was extracted with EtOAc (5×20 mL). The combined organic phases were washed with brine (100 mL), dried (MgSO₄), filtered, and concentrated. The residue was purified by reverse phase chromatography (80 g cartridge) eluting with water (10 mM (NH₄)(HCO₃)) and MeCN (0-100%) to provide a solid. The solid was suspended in TFA (2 mL) and stirred at 23° C. for 2 h. The reaction was neutralized with the dropwise addition of 2.67 M aq. NaOH (10 mL) at 0° C. Water (40 mL) was added. The aq. phase was extracted with EtOAc (4×20 mL). The combined organic phases were washed with brine (50 mL), dried (MgSO₄), filtered, and concentrated. The residue was purified by reverse phase chromatography (80 g cartridge) eluting with water (10 mM (NH₄)(HCO₃)) and MeCN (0-100%) to provide the title compound as a solid (152 mg, 17%). ¹H NMR (400 MHz, DMSO-d6) δ 12.87 (s, 1H), 7.76 (d, J=8.0 Hz, 1H), 7.65 (d, J=8.1 Hz, 1H), 7.37 (dd, J=11.2, 4.0 Hz, 1H), 7.33-7.28 (m, 1H), 7.25 (d, J=2.7 Hz, 2H), 4.15 (d, J=5.2 Hz, 3H).

### Step 4: N-[5-(1H-benzimidazol-2-yl)-1-methyl-1,2,4-triazol-3-yl]-3-chloro-4-methoxy-benzamide

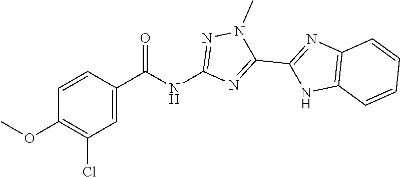 [SMILES: COc1ccc(C(=O)Nc2nc(-c3nc4ccccc4[nH]3)n(C)n2)cc1Cl]

Oxalyl chloride (49.5 μL, 0.585 mmol) and DMF (1.37 μL, 17.7 μmol) were added at 0° C. to a mixture of 3-chloro-4-methoxy-benzoic acid (33.1 mg, 0.177 mmol) in DCM (4 mL). The mixture was warmed to 23° C., stirred for 1 h, and concentrated. The residue was dissolved in THF (4 mL) and pyridine (57.8 μL, 0.717 mmol) was added at 0° C. The mixture was stirred for 10 minutes. 5-(1H-benzimidazol-2-yl)-1-methyl-1,2,4-triazol-3-amine (33.1 mg, 0.177 mmol) was added. The mixture was stirred at 23° C. for 20 h. 0.1 M aq. HCl (5 mL) and water (30 mL) were added. The aqueous phase was extracted with EtOAc (3×15 mL). The combined organic phases were washed with sat. aq. NaHCO₃ (30 mL), brine (30 mL), dried over MgSO₄, filtered, and concentrated. The residue was purified by Gilson HPLC (BEH C18 30×150) eluting with water (10 mM (NH₄)(HCO₂)) and MeCN (37-47%) to provide the formate salt of the title compound as a solid. The solid was dissolved in EtOAc (10 mL), washed with 0.1 M aq. NaHCO₃ (5 mL), dried (MgSO₄), filtered, and concentrated to provide the title compound as a solid (2.40 mg, 4%). ¹H NMR (400 MHz, DMSO-d6) δ 11.22-10.89 (m, 1H), 8.12 (d, J=2.2 Hz, 1H), 8.04 (dd, J=8.7, 2.2 Hz, 1H), 7.50 (s, 2H), 7.29 (d, J=8.7 Hz, 1H), 6.97 (s, 2H), 4.39 (s, 3H), 3.95 (s, 3H). m/z (ES⁺), [M−HCO₂]⁺ 383.2. HPLC (A05) t_{R}=2.22 min.

### EXAMPLE B49

### Preparation of Compound A-235

### Step 1: N-[5-(1H-benzimidazol-2-yl)-1-methyl-pyrazol-3-yl]-4-(4-methyl-3-oxo-piperazin-1-yl)benzamide

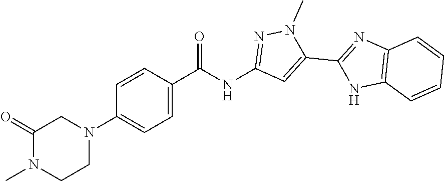 [SMILES: CN1CCN(c2ccc(C(=O)Nc3cc(-c4nc5ccccc5[nH]4)n(C)n3)cc2)CC1=O]

A mixture of 1-methylpiperazin-2-one (340 mg, 2.98 mmol), N-[5-(1H-benzimidazol-2-yl)-1-methyl-pyrazol-3-yl]-4-fluoro-benzamide (50 mg, 0.149 mmol) and DMSO (0.100 mL) was heated at 90° C. for 18 h. The mixture was cooled to 23° C. and water (10 mL) was added. The aq. phase was extracted with EtOAc (3×15 mL). The combined organic phases were washed with brine (50 mL), dried (MgSO₄), filtered, and concentrated. The residue was purified by Gilson HPLC (BEH C18 30×150) eluting with water (10 mM (NH₄)(HCO₃)) and MeCN (24-34%) to provide the title compound as a solid (4.06 mg, 6%). ¹H NMR (400 MHz, DMSO-d6) δ 13.10-13.00 (m, 1H), 10.74 (s, 1H), 7.99 (d, J=9.1 Hz, 2H), 7.76-7.68 (m, 1H), 7.57-7.49 (m, 1H), 7.48 (s, 1H), 7.25 (s, 2H), 6.99 (d, J=9.1 Hz, 2H), 4.30 (s, 3H), 3.91 (s, 2H), 3.63 (d, J=5.9 Hz, 2H), 3.49-3.43 (m, 2H), 2.92 (s, 3H). m/z (ES⁺), [M+H]⁺ 430.3. HPLC (A05) t_{R}=2.07 min.

### EXAMPLE B50

### Preparation of Compound A-236

### Step 1: N-[5-(1H-benzimidazol-2-yl)-1-methyl-pyrazol-3-yl]-6-chloro-2-methyl-pyridine-3-carboxamide

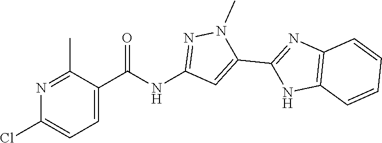 [SMILES: Cc1nc(Cl)ccc1C(=O)Nc1cc(-c2nc3ccccc3[nH]2)n(C)n1]

6-chloro-2-methyl-pyridine-3-carboxylic acid (80.5 mg, 0.469 mmol) and HATU (0.178 g, 0.469 mmol) were dissolved in DMF (3 mL) at 0° C. DIEA (0.161 mL, 0.938 mmol) was added, and the mixture was stirred at 0° C. for 5 min. 5-(1H-benzimidazol-2-yl)-1-methyl-pyrazol-3-amine (100 mg, 0.469 mmol) was added. The mixture was warmed to 23° C. and stirred for 18 h. The mixture was concentrated, and the residue was purified by silica gel chromatography (12 g cartridge) eluting with DCM and MeOH (0-5%). EtOAc (150 mL) was added to the residue and the organic phase was washed with sat. NaHCO₃ (50 mL), water (50 mL) and brine (50 mL), dried (MgSO₄), filtered and concentrated to provide the title compound as a solid (138 mg, 80%). ¹H NMR (400 MHz, DMSO-d6) δ 13.08 (s, 1H), 11.23 (s, 1H), 7.96 (d, J=8.1 Hz, 1H), 7.72 (d, J=7.6 Hz, 1H), 7.56-7.51 (m, 1H), 7.50 (s, 1H), 7.46 (d, J=8.0 Hz, 1H), 7.31-7.20 (m, 2H), 4.30 (s, 3H), 2.57 (s, 3H). m/z (ES⁺), [M+H]⁺ 367.2. HPLC (A05) t_{R}=2.15 min.

### Step 2: N-[5-(1H-benzimidazol-2-yl)-1-methyl-pyrazol-3-yl]-2-methyl-6-morpholino-pyridine-3-carboxamide

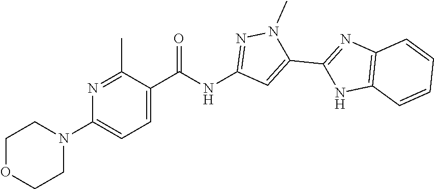 [SMILES: Cc1nc(N2CCOCC2)ccc1C(=O)Nc1cc(-c2nc3ccccc3[nH]2)n(C)n1]

Morpholine (15.4 μL, 0.177 mmol) was added to a solution of N-[5-(1H-benzimidazol-2-yl)-1-methyl-pyrazol-3-yl]-6-chloro-2-methyl-pyridine-3-carboxamide (65 mg, 0.177 mmol) and DIEA (0.0607 mL, 0.354 mmol) in DMSO (1 mL). The mixture was heated to 120° C. and stirred for 18 h. Water (20 mL) was added, and the precipitate was filtered. The residue was purified by silica gel chromatography (12 g cartridge) eluting with DCM and MeOH (0-5%). The product was further purified by a preparative HPLC (BEH C18 30×150) eluting with water (10 mM (NH₄)(HCO₃)) and MeCN (35-45%) to provide the title compound as a solid (28.6 mg, 39%). ¹H NMR (400 MHz, DMSO-d6) δ 13.00 (s, 1H), 10.71 (s, 1H), 7.72 (d, J=8.7 Hz, 1H), 7.70-7.58 (m, 1H), 7.48 (dd, J=17.3, 9.7 Hz, 1H), 7.43 (s, 1H), 7.25-7.15 (m, 2H), 6.65 (d, J=8.5 Hz, 1H), 4.24 (s, 3H), 3.67-3.63 (m, 4H), 3.51-3.48 (m, 4H), 2.47 (s, 3H). m/z (ES⁺), [M+H]⁺ 418.4. HPLC (A05) t_{R}=2.14 min.

### EXAMPLE B51

### Preparation of Compound A-237

### Step 1: N-[5-(7-fluoro-5-methoxy-1H-benzimidazol-2-yl)-1-methyl-pyrazol-3-yl]-6-[2-(hydroxymethyl)morpholin-4-yl]pyridine-3-carboxamide

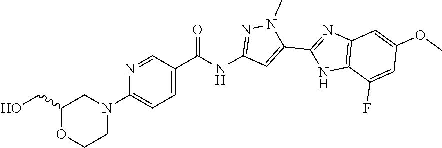 [SMILES: COc1cc(F)c2[nH]c(-c3cc(NC(=O)c4ccc(N5CCOC(CO)C5)nc4)nn3C)nc2c1]

Morpholin-2-ylmethanol (19.3 mg, 0.165 mmol) was added to a solution of 6-chloro-N-[5-(7-fluoro-5-methoxy-1H-benzimidazol-2-yl)-1-methyl-pyrazol-3-yl]pyridine-3-carboxamide (60 mg, 0.150 mmol) in DMSO (1 mL). The mixture was heated to 110° C. and stirred for 18 h. EtOAc (60 mL) was added, and the organic phase was washed with water (3×40 mL) and brine (50 mL), dried (MgSO₄), filtered, and concentrated. The residue was purified by silica gel chromatography (12 g cartridge) eluting with DCM and MeOH (0-5%) to provide the title compound as a solid (56.6 mg, 78%). ¹H NMR (400 MHz, DMSO-d6) δ 13.14 (s, 1H), 10.82 (s, 1H), 8.77 (d, J=2.5 Hz, 1H), 8.13 (dd, J=9.0, 2.5 Hz, 1H), 7.40 (s, 1H), 6.84 (d, J=9.1 Hz, 1H), 6.78 (d, J=1.9 Hz, 1H), 6.69 (dd, J=12.7, 2.5 Hz, 1H), 4.77 (t, J=5.3 Hz, 1H), 4.36-4.28 (m, 1H), 4.22 (s, 3H), 4.18-4.10 (m, 1H), 3.95-3.86 (m, 1H), 3.79 (s, 3H), 3.53-3.38 (m, 4H), 2.96-2.86 (m, 1H), 2.65 (dd, J=13.1, 9.4 Hz, 1H). ¹⁹F NMR (376 MHz, DMSO-d6) δ −127.04 (s). m/z (ES⁺), [M+H]⁺ 482.2. HPLC (A05) t_{R}=2.06 min.

### EXAMPLE B52

### Preparation of Compound A-238

### Step 1: N-[5-(1H-benzimidazol-2-yl)-1-methyl-pyrazol-3-yl]-6-[3-(hydroxymethyl)morpholin-4-yl]pyridine-3-carboxamide

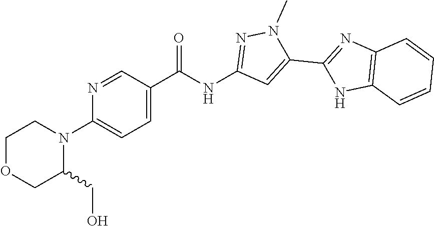 [SMILES: Cn1nc(NC(=O)c2ccc(N3CCOCC3CO)nc2)cc1-c1nc2ccccc2[nH]1]

Morpholin-3-ylmethanol (120 mg, 1.02 mmol) was added to a solution of N-[5-(1H-benzimidazol-2-yl)-1-methyl-pyrazol-3-yl]-6-chloro-pyridine-3-carboxamide (120 mg, 0.340 mmol) in DMSO (2.25 mL). The mixture was heated to 110° C. and stirred for 96 h. EtOAc (60 mL) was added, and the organic phase was washed with water (3×40 mL) and brine (50 mL), dried (MgSO₄), filtered, and concentrated. The residue was purified by silica gel chromatography (12 g cartridge) eluting with DCM and MeOH (0-5%) followed by a preparative HPLC purification (BEH C18 30 ×150) eluting with water (10 mM (NH₄)(HCO₃)) and MeCN (24-34%) to provide the title compound as a solid (43.5 mg, 30%). ¹H NMR (500 MHz, DMSO-d6) δ 13.05 (s, 1H), 10.83 (s, 1H), 8.80 (d, J=2.2 Hz, 1H), 8.16 (dd, J=9.1, 2.5 Hz, 1H), 7.77-7.62 (m, 1H), 7.62-7.50 (m, 1H), 7.47 (s, 1H), 7.30-7.18 (m, 2H), 6.84 (d, J=9.1 Hz, 1H), 4.87 (dd, J=6.3, 4.9 Hz, 1H), 4.30 (s, 3H), 4.25-4.18 (m, 1H), 4.11 (d, J=14.6 Hz, 1H), 4.07 (d, J=11.6 Hz, 1H), 3.92 (dd, J=11.3, 3.7 Hz, 1H), 3.72 (td, J=10.1, 6.4 Hz, 1H), 3.52 (dd, J=11.5, 3.0 Hz, 1H), 3.47 (td, J=11.6, 3.0 Hz, 1H), 3.40 (dt, J=10.1, 5.1 Hz, 1H), 3.09 (td, J=13.1, 3.9 Hz, 1H). m/z (ES⁺), [M+H]⁺ 434.3. HPLC (A05) t_{R}=2.02 min.

### EXAMPLE B53

### Preparation of Compound A-239

### Step 1: N-[5-(7-fluoro-5-methoxy-1H-benzimidazol-2-yl)-1-[(4-methoxyphenyl)-methyl]pyrazol-3-yl]-6-[2-(hydroxymethyl)morpholin-4-yl]pyridine-3-carboxamide

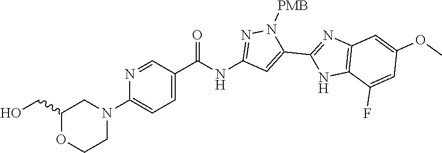

Morpholin-2-ylmethanol (25.4 mg, 0.217 mmol) was added to a solution of 6-chloro-N-[5-(7-fluoro-5-methoxy-1H-benzimidazol-2-yl)-1-[(4-methoxyphenyl)methyl]pyrazol-3-yl]pyridine-3-carboxamide (100 mg, 0.197 mmol) and DIEA (0.0675 mL, 0.395 mmol) in DMSO (1.50 mL). The mixture was heated to 110° C. and stirred for 18 h. Water (10 mL) was added, and the precipitate was filtered. The residue was purified by silica gel chromatography (12 g cartridge) eluting with DCM and MeOH (0-5%) to provide the title compound as a solid (90 mg, 78%). ¹H NMR (400 MHz, DMSO-d6) δ 13.24 (s, 1H), 10.91 (s, 1H), 8.80 (d, J=2.5 Hz, 1H), 8.17 (dd, J=9.0, 2.5 Hz, 1H), 7.51 (s, 1H), 7.24 (d, J=8.7 Hz, 2H), 6.90-6.82 (m, 4H), 6.75 (d, J=12.0 Hz, 1H), 5.96 (s, 2H), 4.81 (t, J=5.5 Hz, 1H), 4.36 (d, J=12.4 Hz, 1H), 4.17 (d, J=12.9 Hz, 1H), 3.95 (dd, J=12.5, 3.3 Hz, 1H), 3.84 (s, 3H), 3.69 (s, 3H), 3.56-3.41 (m, 4H), 2.95 (td, J=13.2, 3.5 Hz, 1H), 2.69 (dd, J=12.6, 9.6 Hz, 1H). ¹⁹F NMR (376 MHz, DMSO-d6) δ −127.19 (s). m/z (ES⁺), [M+H]⁺ 588.4. HPLC (A05) t_{R}=2.30 min.

### Step 2: N-[5-(7-fluoro-5-methoxy-1H-benzimidazol-2-yl)-1H-pyrazol-3-yl]-6-[2-(hydroxymethyl) morpholin-4-yl]pyridine-3-carboxamide

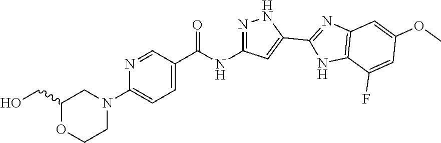 [SMILES: COc1cc(F)c2[nH]c(-c3cc(NC(=O)c4ccc(N5CCOC(CO)C5)nc4)n[nH]3)nc2c1]

TFA (1.50 mL) was added to N-[5-(7-fluoro-5-methoxy-1H-benzimidazol-2-yl)-1-[(4-methoxyphenyl)methyl]pyrazol-3-yl]-6-[2-(hydroxymethyl)morpholin-4-yl]pyridine-3-carboxamide (90 mg, 0.153 mmol), and the mixture was stirred at 23° C. for 1 h. The mixture was concentrated. The residue was dissolved in DMSO (0.600 mL). NEt₃ (0.600 mL) was added, and the mixture was stirred at 23° C. for 30 min. Water (10 mL) was added. The precipitate was filtered, washed with water (10 mL), acetone (10 mL), DCM (10 mL) and acetone (10 mL). The solid was purified by preparative HPLC purification (BEH C18 30 ×150) eluting with water (10 mM (NH₄)(HCO₃)) and MeCN (27-37%) to provide the title compound as a solid (38.3 mg, 56%). ¹H NMR (400 MHz, DMSO-d6) δ 13.41 (s, 1H), 13.09 (s, 1H), 10.78 (s, 1H), 8.76 (d, J=2.3 Hz, 1H), 8.12 (d, J=8.5 Hz, 1H), 7.34 (s, 1H), 6.86 (d, J=6.3 Hz, 1H), 6.79 (s, 1H), 6.73-6.53 (m, 1H), 4.78 (s, 1H), 4.33 (d, J=12.6 Hz, 1H), 4.15 (d, J=12.1 Hz, 1H), 3.91 (dd, J=11.6, 2.3 Hz, 1H), 3.77 (s, 3H), 3.54-3.39 (m, 4H), 2.98-2.84 (m, 1H), 2.64 (d, J=15.7 Hz, 1H). m/z (ES⁺), [M+H]⁺ 468.8. HPLC (A05) t_{R}=1.98 min.

### EXAMPLE B54

### Preparation of Compound A-240

### Step 1: 6-(4-acetylpiperazin-1-yl)-N-[5-(7-fluoro-5-methoxy-1H-benzimidazol-2-yl)-1-methyl-pyrazol-3-yl]pyridine-3-carboxamide

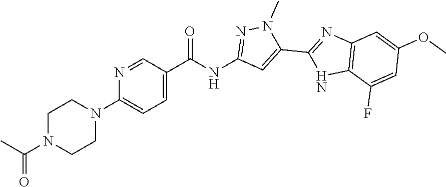 [SMILES: COc1cc(F)c2[nH]c(-c3cc(NC(=O)c4ccc(N5CCN(C(C)=O)CC5)nc4)nn3C)nc2c1]

1-piperazin-1-ylethanone (21.1 mg, 0.165 mmol) was added to a solution of 6-chloro-N-[5-(7-fluoro-5-methoxy-1H-benzimidazol-2-yl)-1-methyl-pyrazol-3-yl]pyridine-3-carboxamide (60 mg, 0.150 mmol) in DMSO (1 mL). The mixture was heated to 110° C. and stirred for 18 h. EtOAc (100 mL) was added, and the organic phase was washed with water (3×40 mL), brine (50 mL), dried (MgSO₄), filtered, and concentrated. The residue was purified by silica gel chromatography (12 g cartridge) eluting with DCM and MeOH (0-10%) to provide the title compound as a solid (44.7 mg, 61%). ¹H NMR (400 MHz, DMSO-d6) δ 13.14 (s, 1H), 10.80 (s, 1H), 8.77 (d, J=2.5 Hz, 1H), 8.13 (dd, J=9.1, 2.5 Hz, 1H), 7.40 (s, 1H), 6.86 (d, J=8.9 Hz, 1H), 6.79 (s, 1H), 6.69 (d, J=12.9 Hz, 1H), 4.22 (s, 3H), 3.78 (s, 3H), 3.69-3.63 (m, 2H), 3.58 (dd, J=6.9, 3.6 Hz, 2H), 3.51 (dd, J=6.7, 3.8 Hz, 4H), 2.01 (s, 3H). ¹⁹F NMR (376 MHz, DMSO-d6) δ −127.06 (s). m/z (ES⁺), [M+H]⁺ 493.4. HPLC (A05) t_{R}=2.09 min.

### EXAMPLE B55

### Preparation of Compound A-241

### Step 1: N-[5-(1H-benzimidazol-2-yl)-1-methyl-1,2,4-triazol-3-yl]-4-fluoro-benzamide

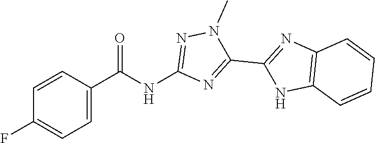 [SMILES: Cn1nc(NC(=O)c2ccc(F)cc2)nc1-c1nc2ccccc2[nH]1]

T3P (50 wt. % in EtOAc, 0.0500 mL, 0.168 mmol) was added dropwise to a mixture of 4-fluorobenzoic acid (23.5 mg, 0.168 mmol), 5-(1H-benzimidazol-2-yl)-1-methyl-1,2,4-triazol-3-amine (Compound A-234) (24 mg, 0.112 mmol) and NEt₃ (0.0187 mL, 0.134 mmol) in EtOAc (1 mL) at 0° C. The mixture was stirred at 23° C. for 1 h and heated at 60° C. for 3 days. More T3P (50 wt. % in EtOAc, 0.0500 mL, 0.168 mmol), 4-fluorobenzoic acid (23.5 mg, 0.168 mmol), and Net₃ (0.0187 mL, 0.134 mmol) were added to the mixture. The mixture was heated at 100° C. under microwave irradiation for 10 h. Water (35 mL) was added, and the aq. phase was extracted with EtOAc (3×10 mL). The combined organic phases were washed with brine (30 mL), dried (MgSO₄), filtered, and concentrated. The residue was purified by silica gel chromatography (12 g cartridge) eluting with DCM and MeCN (0-50%) and by reverse phase chromatography (16 g cartridge) eluting with water (10 mM (NH₄)(HCO₃)) and MeCN (5-100%) to provide the title compound as a solid (5.00 mg, 13%). ¹H NMR (400 MHz, DMSO-d6) δ 11.17-10.87 (m, 1H), 8.10 (dd, J=8.8, 5.6 Hz, 2H), 7.46 (dd, J=6.0, 3.2 Hz, 2H), 7.31 (t, J=8.8 Hz, 2H), 6.91-6.82 (m, 2H), 4.37 (s, 3H). m/z (ES⁺), [M+H]⁺ 337.6. HPLC (A05) t_{R}=2.30 min.

### Step 2: N-[5-(1H-benzimidazol-2-yl)-1-methyl-1,2,4-triazol-3-yl]-4-(4-methylpiperazin-1-yl)benzamide

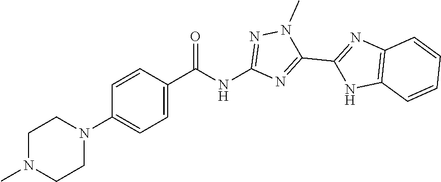 [SMILES: CN1CCN(c2ccc(C(=O)Nc3nc(-c4nc5ccccc5[nH]4)n(C)n3)cc2)CC1]

A mixture of 1-methylpiperazine (59.6 mg, 0.595 mmol), N-[5-(1H-benzimidazol-2-yl)-1-methyl-1,2,4-triazol-3-yl]-4-fluoro-benzamide (4.00 mg, 0.0119 mmol) and DMSO (0.500 mL) was heated at 120° C. for 3 days. The mixture was cooled to 23° C. and concentrated. The residue was purified by Gilson HPLC (BEH C18 30×150) eluting with water (10 mM (NH₄)(HCO₃)) and MeCN (28-38%) to provide the title compound as a solid (0.94 mg, 19%). ¹H NMR (400 MHz, DMSO-d6) δ 13.55-13.14 (m, 1H), 10.46 (s, 1H), 7.89 (d, J=9.0 Hz, 2H), 7.84-7.73 (m, 1H), 7.63-7.51 (m, 1H), 7.30 (s, 2H), 7.01 (d, J=9.1 Hz, 2H), 4.38 (s, 3H), 3.29 (s, 4H), 2.47-2.41 (m, 4H), 2.23 (s, 3H). m/z (ES⁺), [M+H]⁺ 417.8. HPLC (A05) t_{R}=1.82 min.

### EXAMPLE B56

### Preparation of Compound A-242

### Step 1: N-[5-(1H-benzimidazol-2-yl)-1-[(4-methoxyphenyl)methyl]pyrazol-3-yl]-4-(4-methyl-3-oxo-piperazin-1-yl)benzamide

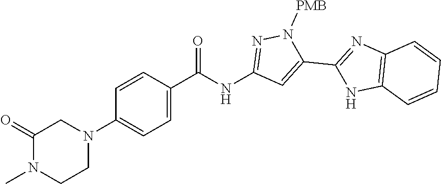

1-methylpiperazin-2-one (367 mg, 3.22 mmol) was added to a solution of N-[5-(1H-benzimidazol-2-yl)-1-[(4-methoxyphenyl)methyl]pyrazol-3-yl]-4-fluoro-benzamide (71 mg, 0.161 mmol) in DMSO (0.100 mL). The mixture was stirred at 120° C. for 120 h. The mixture was cooled to 23° C. and diluted with water (40 mL). The aq. phase was extracted with EtOAc (4×10 mL). The combined organic phases were washed with aq. sat. NaHCO₃ (50 mL), brine (50 mL), dried (MgSO₄), filtered, and concentrated. The residue was purified by silica gel chromatography (12 g cartridge) eluting with MeCN (0-100%) and DCM to provide the title compound as a solid (20 mg, 23%). ¹H NMR (400 MHz, DMSO-d6) δ 13.11 (s, 1H), 10.78 (s, 1H), 7.98 (d, J=9.0 Hz, 2H), 7.71 (d, J=7.8 Hz, 1H), 7.54 (d, J=8.9 Hz, 2H), 7.30-7.20 (m, 4H), 6.97 (d, J=9.1 Hz, 2H), 6.85 (d, J=8.8 Hz, 2H), 6.02 (s, 2H), 3.90 (s, 2H), 3.68 (s, 2H), 3.62 (d, J=6.1 Hz, 3H), 3.48-3.42 (m, 2H), 2.91 (s, 3H). m/z (ES⁺), [M+H]⁺ 536.4. HPLC (A05) t_{R}=2.30 min.

### Step 2: N-[5-(1H-benzimidazol-2-yl)-1H-pyrazol-3-yl]-4-(4-methyl-3-oxo-piperazin-1-yl)benzamide

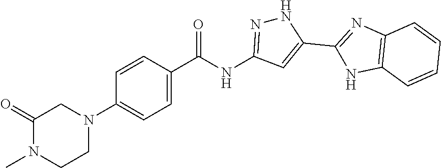 [SMILES: CN1CCN(c2ccc(C(=O)Nc3cc(-c4nc5ccccc5[nH]4)[nH]n3)cc2)CC1=O]

TFA (0.5 mL) was added to N-[5-(1H-benzimidazol-2-yl)-1-[(4-methoxyphenyl) methyl]-pyrazol-3-yl]-4-(4-methyl-3-oxo-piperazin-1-yl)benzamide (20 mg, 0.0373 mmol), and the mixture was stirred at 23° C. for 1.5 h. The mixture was concentrated. The residue was dissolved in DMSO (0.500 mL). NEt₃ (0.500 mL) was added, and the mixture was stirred at 23° C. for 5 min. Water (10 mL) was added, and the mixture was stirred for 5 min. The solid was filtered, dried (6.63 mg), and purified by Gilson HPLC (BEH C18 30×150) eluting with water (10 mM (NH₄)(HCO₃)) and MeCN (26-36%) to provide the title compound as a solid (1.04 mg, 7%). ¹H NMR (500 MHz, DMSO-d6) δ 13.55-13.33 [12.81-12.62] (m, 1H), 13.19-12.91 (m, 1H), 11.06-10.69 [10.96] (m, 1H), 7.97 (d, J=8.5 Hz, 2H), 7.64 (m, 2H), 7.46-7.35 [6.78-6.56] (m, 1H), 7.21 (s, 2H), 7.01 (s, 2H), 3.92 (s, 2H), 3.64 (s, 2H), 3.50-3.43 (m, 2H), 2.92 (s, 3H). m/z (ES⁺), [M+H]⁺ 416.7. HPLC (A05) t_{R}=1.98 min.

### EXAMPLE B57

### Preparation of Compound A-243

### Step 1: N-[5-(1H-benzimidazol-2-yl)-1-[(4-methoxyphenyl)methyl]pyrazol-3-yl]-6-(4-methyl-3-oxo-piperazin-1-yl)pyridine-3-carboxamide

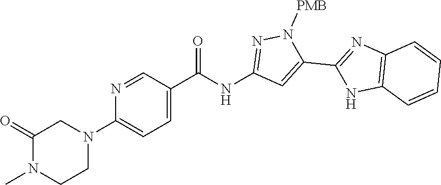

DIEA (51.9 μL, 0.298 mmol) was added to a solution of N-[5-(1H-benzimidazol-2-yl)-1-methyl-pyrazol-3-yl]-6-chloro-pyridine-3-carboxamide (68.4 mg, 0.149 mmol) and 1-methylpiperazin-2-one (27.2 mg, 0.239 mmol) in DMF (1 mL). The mixture was heated at 90° C. for 18 h. The mixture was cooled to 23° C., and water (10 mL) was added. The aq. phase was extracted with EtOAc (3×15 mL). The combined organic phases were washed with brine (50 mL), dried (MgSO₄), filtered, and concentrated. The residue was purified by silica gel chromatography (12 g cartridge) eluting with DCM and MeOH (0-10%) and by Gilson HPLC (BEH C18 30×150) eluting with water (10 mM (NH₄)(HCO₃)) and MeCN (42-52%) to provide the title compound as a solid (20 mg, 25%). ¹H NMR (400 MHz, DMSO-d6) δ 13.15-13.09 (m, 1H), 10.91 (s, 1H), 8.82 (d, J=2.5 Hz, 1H), 8.22-8.14 (m, 1H), 7.77-7.64 (m, 1H), 7.54 (s, 2H), 7.23 (d, J=8.8 Hz, 4H), 6.90 (d, J=9.4 Hz, 1H), 6.85 (d, J=8.8 Hz, 2H), 6.03 (s, 2H), 4.17 (s, 2H), 3.91 (d, J=5.6 Hz, 2H), 3.68 (s, 3H), 3.44 (d, J=5.6 Hz, 2H), 2.90 (s, 3H). m/z (ES⁺), [M+H]⁺ 537.9. HPLC (A05) t_{R}=2.26 min.

### Step 2: N-[5-(1H-benzimidazol-2-yl)-1H-pyrazol-3-yl]-6-(4-methyl-3-oxo-piperazin-1-yl)pyridine-3-carboxamide

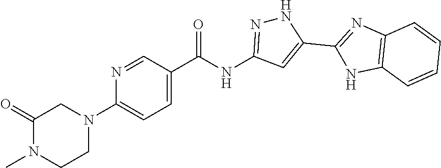 [SMILES: CN1CCN(c2ccc(C(=O)Nc3cc(-c4nc5ccccc5[nH]4)[nH]n3)cn2)CC1=O]

TFA (0.5 mL) was added to N-[5-(1H-benzimidazol-2-yl)-1-[(4-methoxyphenyl) methyl]-pyrazol-3-yl]-6-(4-methyl-3-oxo-piperazin-1-yl)pyridine-3-carboxamide (20 mg, 0.0373 mmol). The mixture was stirred at 23° C. for 1.5 h and concentrated. The residue was dissolved in DMSO (0.500 mL). NEt₃ (0.500 mL) was added, and the mixture was stirred at 23° C. for 5 min. Water (10 mL) was added, and the mixture was stirred for 5 min. The solid was filtered, dried, and purified by Gilson HPLC (BEH C18 30×150) eluting with water (10 mM (NH₄)(HCO₃)) and MeCN (41-51%) to provide the title compound as a solid (2.47 mg, 16%). ¹H NMR (500 MHz, DMSO-d6) δ 13.64-12.72 (m, 2H), 11.26-10.63 (m, 1H), 8.83 (d, J=2.2 Hz, 1H), 8.19 (dd, J=9.0, 2.5 Hz, 1H), 7.58 (s, 2H), 7.47-7.26 (m, 1H), 7.21 (d, J=2.9 Hz, 2H), 6.93 (d, J=9.0 Hz, 1H), 4.18 (s, 2H), 3.97-3.90 (m, 2H), 3.49-3.41 (m, 2H), 2.91 (s, 3H). m/z (ES⁺), [M+H]⁺ 417.4. HPLC (A05) t_{R}=1.93 min.

### EXAMPLE B58

### Preparation of Compound A-244

### Step 1: N-[5-(1H-benzimidazol-2-yl)-1-methyl-pyrazol-3-yl]-6-[4-(2-methylpropanoyl)piperazin-1-yl]pyridine-3-carboxamide

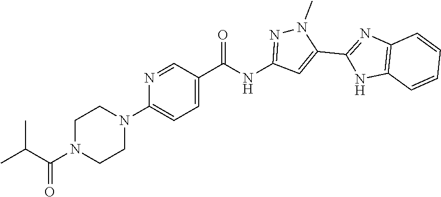 [SMILES: CC(C)C(=O)N1CCN(c2ccc(C(=O)Nc3cc(-c4nc5ccccc5[nH]4)n(C)n3)cn2)CC1]

DIEA (49.4 μL, 0.283 mmol) was added to a solution of 2-methyl-1-piperazin-1-yl-propan-1-one (35.4 mg, 0.227 mmol) and N-[5-(1H-benzimidazol-2-yl)-1-methyl-pyrazol-3-yl]-6-chloro-pyridine-3-carboxamide (50 mg, 0.142 mmol) in DMF (1 mL). The mixture was heated at 90° C. for 40 h. The mixture was cooled to 23° C. and concentrated. The residue was purified by Gilson HPLC (BEH C18 30×150) eluting with water (10 mM (NH₄)(HCO₃)) and MeCN (28-38%) to provide the title compound as a solid (6.91 mg, 10%). ¹H NMR (400 MHz, DMSO-d6) δ 11.19-10.48 (m, 1H), 8.82 (d, J=2.4 Hz, 1H), 8.18 (dd, J=9.0, 2.5 Hz, 1H), 7.56 (d, J=3.2 Hz, 2H), 7.37 (s, 1H), 7.14 (s, 2H), 6.91 (d, J=9.1 Hz, 1H), 4.32 (d, J=7.1 Hz, 3H), 3.64 (m, 8H), 2.92 (dt, J=13.5, 6.8 Hz, 1H), 1.04 (s, 3H), 1.02 (s, 3H). m/z (ES⁺), [M+H]⁺ 473.4 HPLC (A05) t_{R}=2.14 min.

### EXAMPLE B59

### Preparation of Compound A-245

### Step 1: N-[5-(1H-benzimidazol-2-yl)-1-methyl-pyrazol-3-yl]-6-[(3S)-3-methylpiperazin-1-yl]pyridine-3-carboxamide

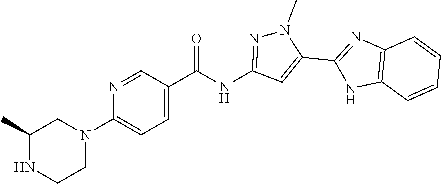 [SMILES: C[C@H]1CN(c2ccc(C(=O)Nc3cc(-c4nc5ccccc5[nH]4)n(C)n3)cn2)CCN1]

Tert-butyl (2S)-2-methylpiperazine-1-carboxylate (37.5 mg, 0.187 mmol) was added to a solution of N-[5-(1H-benzimidazol-2-yl)-1-methyl-pyrazol-3-yl]-6-chloro-pyridine-3-carboxamide (60 mg, 0.170 mmol) in DMSO (1.20 mL). The mixture was heated at 110° C. for 48 h. EtOAc (60 mL) was added, and the organic phase was washed with water (3×40 mL), brine (50 mL), dried (MgSO₄), filtered, and concentrated. MeOH (2 mL) and 1M aq. HCl (1 mL) were added to the residue, and the mixture was stirred for 18 h at 23° C. The mixture was concentrated and the residue was purified by preparative HPLC purification (BEH C18 30 ×150) eluting with water (10 mM (NH₄)(HCO₂)) and MeCN (17-27%) to provide the formate salt of the title compound as a solid (35.8 mg, 46%). ¹H NMR (400 MHz, DMSO-d6) δ 10.76 (s, 1H), 8.73 (d, J=2.5 Hz, 1H), 8.35 (br, 2H), 8.09 (dd, J=9.1, 2.5 Hz, 1H), 7.66-7.51 (m, 2H), 7.42 (s, 1H), 7.24-7.15 (m, 2H), 6.82 (d, J=9.2 Hz, 1H), 4.27-4.18 (m, 5H), 2.93 (d, J=12.1 Hz, 1H), 2.79 (t, J=11.7 Hz, 1H), 2.70-2.60 (m, 2H), 2.41 (dd, J=5.4, 4.5 Hz, 1H), 1.00 (d, J=6.1 Hz, 3H). m/z (ES⁺), [M−HCO₂]⁺ 417.8. HPLC (A05) t_{R}=1.85 min.

### EXAMPLE B60

### Preparation of Compound A-246

### Step 1: N-[5-(1H-benzimidazol-2-yl)-1-methyl-pyrazol-3-yl]-6-(4-methyl-3-oxo-piperazin-1-yl)pyridine-3-carboxamide

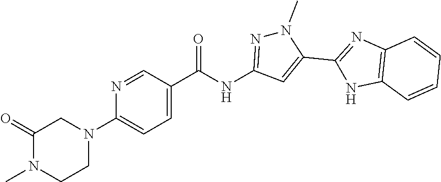 [SMILES: CN1CCN(c2ccc(C(=O)Nc3cc(-c4nc5ccccc5[nH]4)n(C)n3)cn2)CC1=O]

DIEA (0.051 mL, 0.298 mmol) was added to a solution of 1-methylpiperazin-2-one (22.1 mg, 2.98 mmol) and N-[5-(1H-benzimidazol-2-yl)-1-methyl-pyrazol-3-yl]-6-chloro-pyridine-3-carboxamide (52.6 mg, 0.149 mmol) in DMF (1 mL). The mixture was heated at 90° C. for 18 h. The mixture was cooled to 23° C., and water (10 mL) was added. The aq. phase was extracted with EtOAc (3×15 mL). The combined organic phases were washed with brine (50 mL), dried (MgSO₄), filtered, and concentrated. The residue was purified by Gilson HPLC (BEH C18 30×100) eluting with water (10 mM (NH₄)(HCO₃)) and MeCN (21-31%) to provide the title compound as a solid (11.60 mg, 18%). ¹H NMR (400 MHz, DMSO-d6) δ 13.15-12.95 (m, 1H), 10.88 (s, 1H), 8.83 (d, J=2.0 Hz, 1H), 8.20 (dd, J=9.0, 2.5 Hz, 1H), 7.62 (s, 2H), 7.47 (s, 1H), 7.24 (d, J=8.9 Hz, 2H), 6.91 (d, J=8.9 Hz, 1H), 4.30 (s, 3H), 4.18 (s, 2H), 3.95-3.91 (m, 2H), 3.47-3.42 (m, 2H), 2.91 (s, 3H). m/z (ES⁺), [M+H]⁺ 431.3. HPLC (A05) t_{R}=2.00 min.

### EXAMPLE B61

### Preparation of Compound A-247

### Step 1: N-[5-(1H-benzimidazol-2-yl)-1-methyl-pyrazol-3-yl]-6-[(3R)-3-methylpiperazin-1-yl]pyridine-3-carboxamide

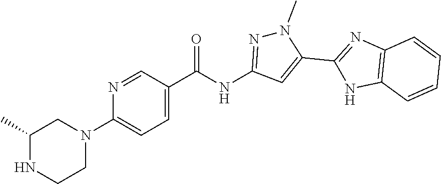 [SMILES: C[C@@H]1CN(c2ccc(C(=O)Nc3cc(-c4nc5ccccc5[nH]4)n(C)n3)cn2)CCN1]

Tert-butyl (2R)-2-methylpiperazine-1-carboxylate (37.5 mg, 0.187 mmol) was added to a solution of N-[5-(1H-benzimidazol-2-yl)-1-methyl-pyrazol-3-yl]-6-chloro-pyridine-3-carboxamide (60 mg, 0.170 mmol) in DMSO (1.20 mL). The mixture was heated to 110° C. and stirred for 48 h. EtOAc (60 mL) was added, and the organic phase was washed with water (3×40 mL), brine (50 mL), dried (MgSO₄), filtered, and concentrated. MeOH (2 mL) and 1M aq. HCl (1 mL) were added to the residue, and the mixture was stirred for 18 h at 23° C. The mixture was concentrated, and the residue was purified by silica gel chromatography (12 g cartridge) eluting with DCM and MeOH (0-100%). The necessary fractions were concentrated. DMSO (0.400 mL) and NEt₃ (0.400 mL) were added to the residue, and the solution was stirred at 23° C. for 1 h. Water (10 mL) was added. The solid was collected by filtration to provide the title compound as a solid (13.2 mg, 19%). ¹H NMR (400 MHz, DMSO-d6) δ 13.05 (s, 1H), 10.80 (s, 1H), 8.78 (d, J=2.5 Hz, 1H), 8.13 (dd, J=9.1, 2.5 Hz, 1H), 7.72 (d, J=7.5 Hz, 1H), 7.53 (d, J=7.6 Hz, 1H), 7.47 (s, 1H), 7.29-7.19 (m, 2H), 6.86 (d, J=9.4 Hz, 1H), 4.31-4.22 (m, 5H), 2.95 (d, J=11.3 Hz, 1H), 2.80 (td, J=12.2, 2.9 Hz, 1H), 2.67 (ddd, J=6.5, 5.6, 2.7 Hz, 2H), 2.44 (dd, J=12.6, 10.3 Hz, 2H), 1.03 (d, J=6.3 Hz, 3H). m/z (ES⁺), [M+H]⁺ 417.5. HPLC (A05) t_{R}=1.85 min.

### EXAMPLE B62

### Preparation of Compound A-248

### Step 1: N-[5-(1H-benzimidazol-2-yl)-1-methyl-pyrazol-3-yl]-6-[(2R)-2-(hydroxymethyl) morpholin-4-yl]pyridine-3-carboxamide

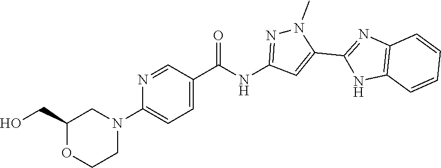 [SMILES: Cn1nc(NC(=O)c2ccc(N3CCO[C@@H](CO)C3)nc2)cc1-c1nc2ccccc2[nH]1]

[(2R)-morpholin-2-yl]methanol hydrochloride (28.7 mg, 0.187 mmol) was added to a solution of N-[5-(1H-benzimidazol-2-yl)-1-methyl-pyrazol-3-yl]-6-chloro-pyridine-3-carboxamide (60 mg, 0.170 mmol) in DMSO (1.20 mL). The mixture was heated to 110° C. and stirred for 18 h. EtOAc (60 mL) was added, and the organic phase was washed with water (3×40 mL), brine (50 mL), dried (MgSO₄), filtered, and concentrated. The residue was purified by silica gel chromatography (12 g cartridge) eluting with DCM and MeOH (0-15%) to provide the title compound as a solid (34.2 mg, 46%). ¹H NMR (400 MHz, DMSO-d6) δ 13.06 (s, 1H), 10.85 (s, 1H), 8.82 (d, J=2.1 Hz, 1H), 8.18 (dd, J=9.0, 2.5 Hz, 1H), 7.83-7.52 (m, 2H), 7.46 (s, 1H), 7.28-7.19 (m, 2H), 6.89 (d, J=9.2 Hz, 1H), 4.82 (t, J=5.4 Hz, 1H), 4.37 (d, J=13.5 Hz, 1H), 4.30 (s, 3H), 4.19 (d, J=13.6 Hz, 1H), 3.95 (d, J=11.6 Hz, 1H), 3.49 (ddd, J=11.0, 8.8, 3.2 Hz, 4H), 2.96 (td, J=13.1, 3.7 Hz, 1H), 2.73-2.67 (m, 1H). m/z (ES⁺), [M+H]⁺ 434.4. HPLC (A05) t_{R}=1.99 min.

### EXAMPLE B63

### Preparation of Compound A-249

### Step 1: 6-[(8aR)-6-oxo-1,3,4,7,8,8a-hexahydropyrrolo[1,2-a]pyrazin-2-yl]-N-[5-(1H-benzimidazol-2-yl)-1-methyl-pyrazol-3-yl]pyridine-3-carboxamide

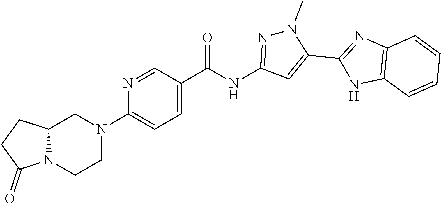 [SMILES: Cn1nc(NC(=O)c2ccc(N3CCN4C(=O)CC[C@@H]4C3)nc2)cc1-c1nc2ccccc2[nH]1]

DIEA (49.4 μL, 0.283 mmol) was added to a solution of (8aR)-2,3,4,7,8,8a-hexahydro-1H-pyrrolo[1,2-a]pyrazin-6-one hydrochloride (40.1 mg, 0.227 mmol) and N-[5-(1H-benzimidazol-2-yl)-1-methyl-pyrazol-3-yl]-6-chloro-pyridine-3-carboxamide (50 mg, 0.142 mmol) in DMF (1 mL). The mixture was heated at 100° C. for one week. The mixture was cooled to 23° C. and water (30 mL) was added. The aq. phase was extracted with EtOAc (3×15 mL), and the combined organic phases were washed with brine (45 mL), dried (MgSO₄), filtered, and concentrated. The residue was purified by silica gel chromatography (12 g cartridge) eluting with DCM and MeCN (0-100%) to provide a solid. The solid was suspended in DMSO (0.500 mL) and water (10 mL). The aq. phase was extracted with EtOAc (3×15 mL). The combined organic phases were washed with sat. aq. NaHCO₃ (30 mL) and concentrated to provide the title compound as a solid (41.32 mg, 64%). ¹H NMR (400 MHz, DMSO-d6) δ 13.71-12.32 (m, 1H), 10.80 (s, 1H), 8.81 (d, J=2.0 Hz, 1H), 8.18 (dd, J=9.0, 2.5 Hz, 1H), 7.57 (dd, J=5.9, 3.2 Hz, 2H), 7.39 (s, 1H), 7.15 (dd, J=6.0, 3.1 Hz, 2H), 6.99 (d, J=8.9 Hz, 1H), 4.65 (d, J=10.8 Hz, 1H), 4.53 (d, J=9.4 Hz, 1H), 4.31 (s, 3H), 3.88 (d, J=9.1 Hz, 1H), 3.62-3.52 (m, 1H), 2.91-2.75 (m, 2H), 2.68 (dd, J=13.0, 11.0 Hz, 1H), 2.34-2.25 (m, 2H), 2.21-2.11 (m, 1H), 1.63 (ddd, J=19.5, 12.5, 9.3 Hz, 1H). m/z (ES⁺), [M+H]⁺ 457.3. HPLC (A05) t_{R}=1.99 min.

### EXAMPLE B64

### Preparation of Compound A-250

### Step 1: 6-[(84-6-oxo-1,3,4,7,8,8a-hexahydropyrrolo[1,2-a]pyrazin-2-yl]-N-[5-(1H-benzimidazol-2-yl)-1-methyl-pyrazol-3-yl]pyridine-3-carboxamide

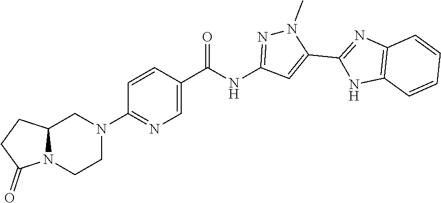 [SMILES: Cn1nc(NC(=O)c2ccc(N3CCN4C(=O)CC[C@H]4C3)nc2)cc1-c1nc2ccccc2[nH]1]

DIEA (49.4 μL, 0.283 mmol) was added to a solution of (8aS)-2,3,4,7,8,8a-hexahydro-1H-pyrrolo[1,2-a]pyrazin-6-one hydrochloride (40.1 mg, 0.227 mmol) and N-[5-(1H-benzimidazol-2-yl)-1-methyl-pyrazol-3-yl]-6-chloro-pyridine-3-carboxamide (50 mg, 0.142 mmol) in DMF (1 mL). The mixture was heated at 100° C. for one week. The mixture was cooled to 23° C. and water (30 mL) was added. The aq. phase was extracted with EtOAc (3×15 mL), and the combined organic phases were washed with brine (45 mL), dried (MgSO₄), filtered, and concentrated. The residue was purified by silica gel chromatography (12 g cartridge) eluting with DCM and MeCN (0-100%) to provide a solid. The solid was suspended in DMSO (0.500 mL) and water (10 mL). The aq. phase was extracted with EtOAc (3×15 mL). The combined organic phases were washed with sat. aq. NaHCO₃ (30 mL) and concentrated to provide the title compound as a solid (45.43 mg, 70%). ¹H NMR (400 MHz, DMSO-d6) δ 10.71 (s, 1H), 8.81 (d, J=2.4 Hz, 1H), 8.22-8.14 (m, 1H), 7.50 (s, 2H), 7.28 (s, 1H), 7.03 (s, 2H), 6.98 (d, J=9.1 Hz, 1H), 4.64 (s, 1H), 4.51 (s, 1H), 4.31 (s, 3H), 3.89 (s, 1H), 3.59 (s, 1H), 2.83 (d, J=7.3 Hz, 2H), 2.73-2.64 (m, 1H), 2.27 (d, J=10.3 Hz, 2H), 2.20-2.11 (m, 1H), 1.65 (s, 1H). m/z (ES⁺), [M+H]⁺ 457.4 HPLC (A05) t_{R}=2.05 min.

### EXAMPLE B65

### Preparation of Compound A-251

### Step 1: N-[5-(1H-benzimidazol-2-yl)-1-methyl-pyrazol-3-yl]-6-[(2S)-2-(hydroxymethyl) morpholin-4-yl]pyridine-3-carboxamide

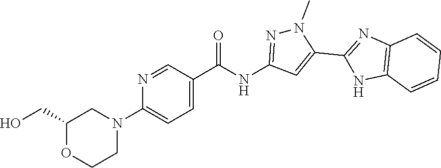 [SMILES: Cn1nc(NC(=O)c2ccc(N3CCO[C@H](CO)C3)nc2)cc1-c1nc2ccccc2[nH]1]

[(2S)-morpholin-2-yl]methanol hydrochloride (28.7 mg, 0.187 mmol) was added to a solution of N-[5-(1H-benzimidazol-2-yl)-1-methyl-pyrazol-3-yl]-6-chloro-pyridine-3-carboxamide (60 mg, 0.170 mmol) in DMSO (1.20 mL). The mixture was heated to 110° C. and stirred for 18 h. EtOAc (60 mL) was added, and the organic phase was washed with water (3×40 mL), brine (50 mL), dried (Na₂SO₄), filtered, and concentrated. The residue was purified twice by silica gel chromatography (12 g cartridge) eluting with DCM and MeOH (0-10%) to provide the title compound as a solid (11.4 mg, 16%). ¹H NMR (400 MHz, DMSO-d6) δ 13.06 (s, 1H), 10.86 (s, 1H), 8.82 (d, J=2.3 Hz, 1H), 8.18 (dd, J=9.0, 2.5 Hz, 1H), 7.76-7.63 (m, 1H), 7.62-7.51 (m, 1H), 7.47 (s, 1H), 7.25 (s, 2H), 6.89 (d, J=9.0 Hz, 1H), 4.82 (t, J=5.4 Hz, 1H), 4.37 (d, J=13.0 Hz, 1H), 4.30 (s, 3H), 4.18 (d, J=13.0 Hz, 1H), 3.96 (d, J=9.1 Hz, 1H), 3.58-3.42 (m, 4H), 3.01-2.90 (m, 1H), 2.70 (dd, J=12.5, 9.5 Hz, 1H). m/z (ES⁺), [M+H]⁺ 434.4. HPLC (A05) t_{R}=1.99 min.

### EXAMPLE B66

### Preparation of Compound A-252

### Step 1: N-[5-(1H-benzimidazol-2-yl)-1-methyl-pyrazol-3-yl]-3-chloro-4-hydroxy-benzamide

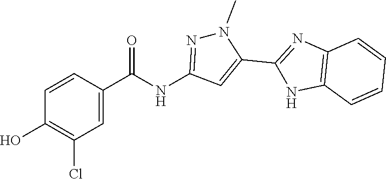 [SMILES: Cn1nc(NC(=O)c2ccc(O)c(Cl)c2)cc1-c1nc2ccccc2[nH]1]

DIEA (0.198 mL, 1.16 mmol) was added to a solution of 3-chloro-4-hydroxy-benzoic acid (100 mg, 0.579 mmol) and HATU (220 mg, 0.579 mmol) in DMF (5 mL) at 0° C. The mixture was stirred at 0° C. for 5 min. 5-(1H-benzimidazol-2-yl)-1-methyl-pyrazol-3-amine (124 mg, 0.579 mmol) was added. The mixture was warmed to 23° C. and stirred for 24 h. The mixture was concentrated. Water (50 mL) was added. The aq. phase was extracted with EtOAc (4×10 mL). The combined organic phases were washed with sat. aq. NaHCO₃ (2×40 mL), brine (40 mL), dried (MgSO₄), filtered, and concentrated. The residue was purified by silica gel chromatography (12 g cartridge) eluting with DCM and MeOH (0-20%) to provide the title compound as a solid (88 mg, 41%). ¹H NMR (400 MHz, DMSO-d6) δ 13.06 (s, 1H), 10.93 (s, 1H), 10.92-10.88 (m, 1H), 8.11 (d, J=2.2 Hz, 1H), 7.90 (dd, J=8.6, 2.2 Hz, 1H), 7.72 (d, J=7.9 Hz, 1H), 7.53 (d, J=7.0 Hz, 1H), 7.47 (s, 1H), 7.29-7.20 (m, 2H), 7.07-7.03 (m, 1H), 4.30 (s, 3H). m/z (ES⁺), [M+H]⁺ 368.6. HPLC (A05) t_{R}=2.14 min.

### Step 2: oxetan-3-yl methanesulfonate

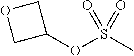

NEt₃ (0.564 mL, 4.05 mmol) was added to a solution of oxetan-3-ol (100 mg, 1.35 mmol) and methanesulfonyl chloride (0.125 mL, 1.62 mmol) in DMF (10 mL). The mixture was stirred at 23° C. for 18 h. Water (100 mL) was added, and the aq. phase was extracted with EtOAc (3×15 mL). The combined organic phases were washed with brine (40 mL), dried (MgSO₄), filtered, and concentrated to provide the title compound as an oil (200 mg, 97%), which was used as such for the next step without further purification. ¹H NMR (300 MHz, CDCl₃) δ 5.55-5.44 (m, 1H), 4.87 (dd, J=7.8, 6.9 Hz, 2H), 4.83-4.76 (m, 2H), 3.05 (s, 3H).

### Step 3: N-[5-(1H-benzimidazol-2-yl)-1-methyl-pyrazol-3-yl]-3-chloro-4-(oxetan-3-yloxy)benzamide

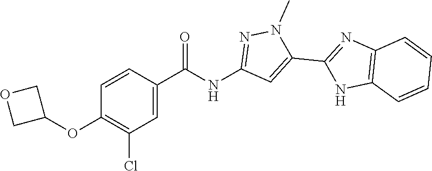 [SMILES: Cn1nc(NC(=O)c2ccc(OC3COC3)c(Cl)c2)cc1-c1nc2ccccc2[nH]1]

A solution of N-[5-(1H-benzimidazol-2-yl)-1-methyl-pyrazol-3-yl]-3-chloro-4-hydroxy-benzamide (29 mg, 0.0788 mmol), oxetan-3-yl methanesulfonate (13.2 mg, 0.173 mmol), and Na₂CO₃ (16.7 mg, 0.158 mmol) in DMF (1 mL) was refluxed for 2 h. After cooling to 23° C., water (30 mL) was added, and the aq. phase was extracted with EtOAc (3×15 mL). The combined organic phases were washed with brine (50 mL), dried (MgSO₄), filtered, and concentrated. The residue was purified by silica gel chromatography (12 g cartridge) eluting with DCM and MeOH (0-10%) to provide a solid. The solid was purified by Gilson HPLC (BEH C18 30×100) eluting with water (10 mM (NH₄)(HCO₂)) and MeCN (34-44%) to provide the title compound as a solid (8.63 mg, 23%). ¹H NMR (400 MHz, DMSO-d6) δ 11.07 (s, 1H), 8.20 (d, J=2.2 Hz, 1H), 8.01 (dd, J=8.6, 2.2 Hz, 1H), 7.63 (s, 2H), 7.48 (s, 1H), 7.25 (dd, J=6.0, 3.1 Hz, 2H), 6.91 (d, J=8.7 Hz, 1H), 5.50-5.43 (m, 1H), 4.99 (t, J=7.1 Hz, 2H), 4.60 (dd, J=8.2, 4.8 Hz, 2H), 4.31 (s, 3H). m/z (ES⁺), [M+H]⁺ 424.3. HPLC (A05) t_{R}=2.16 min.

### EXAMPLE B67

### Preparation of Compound A-253

### Step 1: N-[5-(1H-benzimidazol-2-yl)-1-methyl-pyrazol-3-yl]-2-methyl-6-(4-methylpiperazin-1-yl)pyridine-3-carboxamide

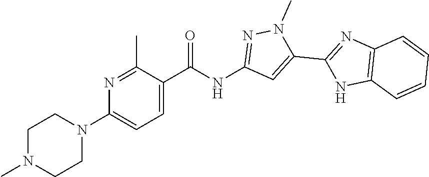 [SMILES: Cc1nc(N2CCN(C)CC2)ccc1C(=O)Nc1cc(-c2nc3ccccc3[nH]2)n(C)n1]

1-methylpiperazine (14.3 μL, 0.180 mmol) was added to a solution of N-[5-(1H-benzimidazol-2-yl)-1-methyl-pyrazol-3-yl]-6-chloro-2-methyl-pyridine-3-carboxamide (Compound A-236) (60 mg, 0.164 mmol) and DIEA (0.0560 mL, 0.327 mmol) in DMSO (1.20 mL). The mixture was heated to 110° C. and stirred for 18 h. EtOAc (60 mL) was added, and the organic phase was washed with water (3×40 mL), brine (50 mL), dried (Na₂SO₄), filtered, and concentrated. The residue was purified by silica gel chromatography (12 g cartridge) eluting with DCM and MeOH (0-20%) to provide the title compound as a solid (44.1 mg, 63%). ¹H NMR (400 MHz, DMSO-d6) δ 13.04 (s, 1H), 10.74 (s, 1H), 7.76 (d, J=8.7 Hz, 1H), 7.71 (d, J=7.3 Hz, 1H), 7.55-7.50 (m, 1H), 7.48 (s, 1H), 7.25 (tdd, J=14.8, 7.2, 1.3 Hz, 2H), 6.71 (d, J=9.0 Hz, 1H), 4.29 (s, 3H), 3.78-3.39 (m, 4H), 2.51 (s, 3H), 2.47-2.15 (m, 5H), 1.25 (dd, J=10.7, 4.8 Hz, 2H). m/z (ES⁺), [M+H]⁺ 431.3. HPLC (A05) t_{R}=1.89 min.

### EXAMPLE B68

### Preparation of Compound A-258

### Step 1: tert-butyl 4-[5-[[5-(1H-benzimidazol-2-yl)-1-methyl-pyrazol-3-yl]carbamoyl]-2-pyridyl]-2-(hydroxymethyl)piperazine-1-carboxylate

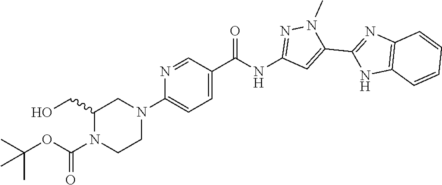 [SMILES: Cn1nc(NC(=O)c2ccc(N3CCN(C(=O)OC(C)(C)C)C(CO)C3)nc2)cc1-c1nc2ccccc2[nH]1]

Tert-butyl 2-(hydroxymethyl)piperazine-1-carboxylate (40.5 mg, 0.187 mmol) and N-[5-(1H-benzimidazol-2-yl)-1-methyl-pyrazol-3-yl]-6-chloro-pyridine-3-carboxamide (60 mg, 0.170 mmol) were dissolved in DMSO (1.20 mL). The mixture was heated to 110° C. and stirred for 18 h. EtOAc (60 mL) was added, and the organic phase was washed with water (3×40 mL), brine (50 mL), dried (Na₂SO₄), filtered, and concentrated. The residue was purified by silica gel chromatography (12 g cartridge) eluting with DCM and MeOH (0-15%) to provide the title compound as a solid (61 mg, 67%). ¹H NMR (400 MHz, DMSO-d6) δ 13.00 (s, 1H), 10.78 (s, 1H), 8.75 (d, J=2.5 Hz, 1H), 8.11 (dd, J=9.1, 2.5 Hz, 1H), 7.64 (dd, J=21.9, 15.4 Hz, 1H), 7.56-7.46 (m, 1H), 7.42 (s, 1H), 7.24-7.14 (m, 2H), 6.79 (d, J=9.0 Hz, 1H), 4.80 (t, J=5.4 Hz, 1H), 4.30 (d, J=12.7 Hz, 1H), 4.25 (s, 3H), 4.19 (d, J=13.2 Hz, 1H), 3.98 (dd, J=13.7, 7.3 Hz, 1H), 3.77 (d, J=13.1 Hz, 1H), 3.39-3.30 (m, 2H), 3.19 (dd, J=13.4, 4.3 Hz, 1H), 3.06 (d, J=12.1 Hz, 1H), 3.02-2.94 (m, 1H), 1.38 (s, 9H). m/z (ES⁺), [M+H]⁺ 533.5. HPLC (A05) t_{R}=2.20 min.

### Step 2: N-[5-(1H-benzimidazol-2-yl)-1-methyl-pyrazol-3-yl]-6-[3-(hydroxy-methyl)piperazin-1-yl]pyridine-3-carboxamide

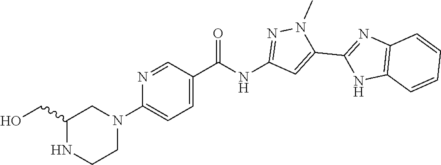 [SMILES: Cn1nc(NC(=O)c2ccc(N3CCNC(CO)C3)nc2)cc1-c1nc2ccccc2[nH]1]

1M Aq. HCl (1 mL, 1 mmol) was added to a solution of tert-butyl 4-[5-[[5-(1H-benzimidazol-2-yl)-1-methyl-pyrazol-3-yl]carbamoyl]-2-pyridyl]-2-(hydroxymethyl)piperazine-1-carboxylate (61 mg, 0.115 mmol) in MeOH (2 mL). The mixture was stirred at 23° C. for 18 h. The mixture was dried to provide the dihydrochloride salt of the title compound as a solid (57 mg, 99%). ¹H NMR (300 MHz, DMSO-d6) δ 11.07 (s, 1H), 9.60 (s, 1H), 9.37 (s, 1H), 8.83 (s, 1H), 8.33-8.21 (m, 1H), 7.73 (d, J=10.3 Hz, 2H), 7.52 (s, 1H), 7.38 (dd, J=17.7, 8.2 Hz, 2H), 7.14-7.05 (m, 1H), 4.52 (dd, J=21.0, 14.3 Hz, 2H), 4.27 (s, 3H), 3.80-3.61 (m, 2H), 3.43-3.16 (m, 4H), 3.17-2.98 (m, 1H). m/z (ES⁺), [M−2Cl]⁺ 434.1. HPLC (A05) t_{R}=1.83 min.

Starting from 1M aq. HCl (3 mL, 3.00 mmol) and tert-butyl 4-[5-[[5-(1H-benzimidazol-2-yl)-1-methyl-pyrazol-3-yl]carbamoyl]-2-pyridyl]-2-(hydroxymethyl)piperazine-1-carboxylate (180 mg, 0.338 mmol) in MeOH (3 mL), the title compound was synthesized as described above. The residue was purified by two preparative HPLC purification (BEH C18 30 ×150) eluting with water (10 mM (NH₄)(HCO₃)) and MeCN (20-30%) followed by water (10 mM (NH₄)(HCO₂)) and MeCN (22-32%) to provide the formate salt of the title compound as a solid (29.2 mg, 18%). ¹H NMR (400 MHz, DMSO-d6) δ 10.85 (s, 1H), 8.79 (d, J=2.4 Hz, 1H), 8.35 (s, 1H), 8.14 (dd, J=9.1, 2.5 Hz, 1H), 7.73-7.53 (m, 2H), 7.47 (s, 1H), 7.28-7.19 (m, 2H), 6.85 (d, J=9.1 Hz, 1H), 4.37 (d, J=12.7 Hz, 1H), 4.30 (s, 3H), 4.23 (d, J=12.1 Hz, 1H), 3.39 (d, J=3.5 Hz, 2H), 3.00 (d, J=8.8 Hz, 1H), 2.87 (t, J=11.5 Hz, 1H), 2.73-2.62 (m, 2H), 2.56 (d, J=8.6 Hz, 1H). m/z (ES⁺), [M−HCO₂]⁺ 433.2. HPLC (A05) t_{R}=1.84 min.

### EXAMPLE B69

### Preparation of Compound A-254

### Step 1: 6-14-acetyl-3-(hydroxymethyl)piperazin-1-yl-N-[5-(1H-benzimidazol-2-yl)-1-methyl-pyrazol-3-yl]pyridine-3-carboxamide

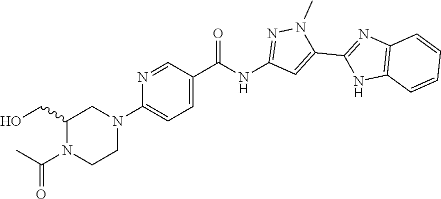 [SMILES: CC(=O)N1CCN(c2ccc(C(=O)Nc3cc(-c4nc5ccccc5[nH]4)n(C)n3)cn2)CC1CO]

Acetyl chloride (6.33 μL, 0.089 mmol) was added to a solution of N-[5-(1H-benzimidazol-2-yl)-1-methyl-pyrazol-3-yl]-6-[3-(hydroxymethyl)piperazin-1-yl]pyridine-3-carboxamide dihydrochloride (Compound A-263) (30 mg, 0.0594 mmol) and DIEA (40.6 μL, 0.237 mmol) in DMF (1 mL). The mixture was stirred at 23° C. for 18 h. The mixture was concentrated. The residue was purified by silica gel chromatography (12 g cartridge) eluting with DCM and MeOH (0-20%) and by preparative HPLC purification (BEH C18 30 ×150) eluting with water (10 mM (NH₄)(HCO₃)) and MeCN (20-30%) to provide the title compound as a solid (13.4 mg, 48%). ¹H NMR (500 MHz, DMSO-d6, 90° C.) δ 10.47 (s, 1H), 8.81 (d, J=2.5 Hz, 1H), 8.33 (s, 1H), 8.17 (dd, J=9.0, 2.5 Hz, 1H), 7.62 (dd, J=5.5, 3.3 Hz, 2H), 7.41 (s, 1H), 7.27-7.22 (m, 2H), 6.84 (d, J=9.0 Hz, 1H), 4.36 (d, J=13.1 Hz, 1H), 4.29 (s, 3H), 4.27-4.04 (m, 3H), 3.49 (d, J=6.6 Hz, 2H), 3.29 (dd, J=13.4, 4.3 Hz, 1H), 3.20-2.98 (m, 2H), 2.08 (s, 3H). m/z (ES⁺), [M+H]⁺ 475.8. HPLC (A05) t_{R}=1.98 min.

### EXAMPLE B70

### Preparation of Compound A-255

### Step 1: N-[5-(1H-benzimidazol-2-yl)-1-methyl-pyrazol-3-yl]-6-[(3R,5S)-3,5-dimethylpiperazin-1-yl]pyridine-3-carboxamide

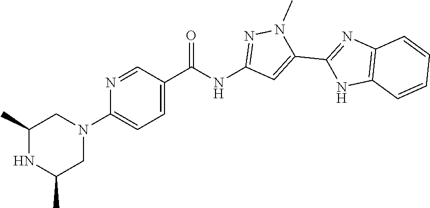 [SMILES: C[C@@H]1CN(c2ccc(C(=O)Nc3cc(-c4nc5ccccc5[nH]4)n(C)n3)cn2)C[C@H](C)N1]

(2R,6S)-2,6-dimethylpiperazine (21.4 mg, 0.187 mmol) was added to a solution of N-[5-(1H-benzimidazol-2-yl)-1-methyl-pyrazol-3-yl]-6-chloro-pyridine-3-carboxamide (60 mg, 0.170 mmol) and DIEA (0.0582 mL, 0.340 mmol) in DMSO (1.20 mL). The mixture was heated to 110° C. and stirred for 18 h. EtOAc (60 mL) was added, and the organic phase was washed with water (3×40 mL), brine (50 mL), dried (MgSO₄), filtered, and concentrated. The residue was purified by silica gel chromatography (12 g cartridge) eluting with DCM and MeOH (0-10%) and by preparative HPLC purification (BEH C18 30×150) eluting with water (10 mM (NH₄)(HCO₃)) and MeCN (27-37%) to provide the title compound as a solid (47.7 mg, 65%). ¹H NMR (400 MHz, DMSO-d6) δ 13.00 (s, 1H), 10.74 (s, 1H), 8.72 (d, J=2.4 Hz, 1H), 8.07 (dd, J=9.1, 2.5 Hz, 1H), 7.66 (d, J=7.6 Hz, 1H), 7.47 (d, J=7.6 Hz, 1H), 7.41 (s, 1H), 7.25-7.14 (m, 2H), 6.82 (d, J=9.1 Hz, 1H), 4.28-4.21 (m, 5H), 2.66 (ddd, J=9.4, 6.3, 3.0 Hz, 2H), 2.28 (dd, J=12.5, 10.7 Hz, 2H), 0.98 (d, J=6.3 Hz, 6H). m/z (ES⁺), [M+H]⁺ 431.4. HPLC (A05) t_{R}=1.89 min.

### EXAMPLE B71

### Preparation of Compound A-256

### Step 1: 5-(2-methoxyethoxy)-2-nitro-aniline

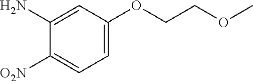

K₂CO₃ (3.54 g, 0.0256 mol) was added to a solution of 5-fluoro-2-nitro-aniline (2 g, 0.0128 mol) in 2-Methoxy-ethanol (20 mL). The mixture was stirred at 110° C. for 18 h. The mixture was concentrated, and the residue was purified by silica gel chromatography (80 g cartridge) eluting with hexanes and EtOAc (0-60%) to provide the title compound as a solid (1.93 g, 71%). ¹H NMR (400 MHz, DMSO-d6) δ 7.91 (d, J=9.5 Hz, 1H), 7.46 (s, 2H), 6.44 (d, J=2.7 Hz, 1H), 6.25 (dd, J=9.5, 2.7 Hz, 1H), 4.13-4.06 (m, 2H), 3.69-3.63 (m, 2H), 3.30 (s, 3H). m/z (ES⁺), [M+H]⁺ 213.6. HPLC (A05) t_{R}=2.07 min.

### Step 2: tert-butyl N-[5-(2-methoxyethoxy)-2-nitro-phenyl]carbamate

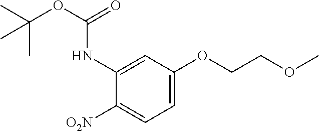 [SMILES: COCCOc1ccc([N+](=O)[O-])c(NC(=O)OC(C)(C)C)c1]

A solution of Boc₂O (0.113 g, 0.518 mmol) in 1,4-Dioxane (1 mL) was added dropwise to a solution of 5-(2-methoxyethoxy)-2-nitro-aniline (100 mg, 0.471 mmol), DMAP (5.76 mg, 0.0471 mmol) and TEA (0.0788 mL, 0.565 mmol) in 1,4-Dioxane (3 mL) at 23° C. The mixture was stirred for 18 h. The mixture was concentrated, and the residue was purified by silica gel chromatography (12 g cartridge) eluting with hexanes and EtOAc (0-100%) to provide the title compound as an oil (47 mg, 32%). ¹H NMR (400 MHz, DMSO-d6) δ 9.72 (s, 1H), 8.07 (d, J=9.3 Hz, 1H), 7.58 (d, J=2.7 Hz, 1H), 6.82 (dd, J=9.4, 2.8 Hz, 1H), 4.23-4.18 (m, 2H), 3.70-3.65 (m, 2H), 3.31 (s, 3H), 1.47 (s, 9H). HPLC (A05) t_{R}=2.56 min.

### Step 3: tert-butyl N-[2-amino-5-(2-methoxyethoxy)phenyl]carbamate

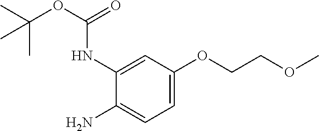 [SMILES: COCCOc1ccc(N)c(NC(=O)OC(C)(C)C)c1]

Tert-butyl N-[5-(2-methoxyethoxy)-2-nitro-phenyl]carbamate (47 mg, 0.15 mmol) was solubilized in EtOH (10 mL) and transferred in a flask purged with nitrogen containing Pd/C (10.0%, 25 mg, 0.0235 mmol). The mixture was stirred at 23° C. for 2 h under an atmosphere of hydrogen. The flask was purged with nitrogen, the solution was filtered on celite, and the filtrate was concentrated to provide the title compound as a solid (42 mg, 99%). ¹H NMR (300 MHz, DMSO-d6) δ 8.27 (s, 1H), 6.94 (d, J=2.5 Hz, 1H), 6.61 (d, J=8.6 Hz, 1H), 6.47 (dd, J=8.6, 2.8 Hz, 1H), 4.44 (s, 2H), 3.95-3.89 (m, 2H), 3.62-3.55 (m, 2H), 3.29 (s, 3H), 1.46 (s, 9H). m/z (ES⁺), [M+H]⁺ 283.6. HPLC (A05) t_{R}=2.09 min.

### Step 4: N tert-butyl N-[2-[[5-[(3-chloro-4-methoxy-benzoyl)amino]-2-[(4-methoxyphenyl) methyl]pyrazole-3-carbonyl]amino]-5-(2-methoxyethoxy)phenyl]carbamate

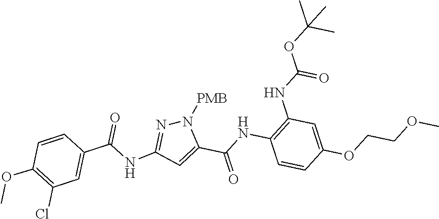

5-[(3-chloro-4-methoxy-benzoyl)amino]-2-[(4-methoxyphenyl)methyl]pyrazole-3-carboxylic acid (61.9 mg, 0.149 mmol) and HATU (56.6 mg, 0.149 mmol) were dissolved in DMF (1 mL) at 0° C. DIEA (0.0509 mL, 0.298 mmol) was added, and the mixture was stirred at 0° C. for 5 min. Tert-butyl N-[2-amino-5-(2-methoxyethoxy)phenyl]carbamate (42 mg, 0.149 mmol) was added. The mixture was warmed to 23° C. and stirred for 18 h. The mixture was concentrated, and the residue was purified by silica gel chromatography (12 g cartridge) eluting with DCM and MeOH (0-10%) to provide the title compound as a solid (96.5 mg, 95%). ¹H NMR (300 MHz, DMSO-d6) δ 11.04 (s, 1H), 9.73 (s, 1H), 8.64 (s, 1H), 8.12 (d, J=2.1 Hz, 1H), 8.03 (dd, J=8.7, 2.1 Hz, 1H), 7.49 (s, 1H), 7.33-7.18 (m, 5H), 6.86 (d, J=8.7 Hz, 2H), 6.71 (dd, J=8.8, 2.7 Hz, 1H), 5.59 (s, 2H), 4.09-4.02 (m, 2H), 3.92 (s, 3H), 3.69 (s, 3H), 3.67-3.62 (m, 2H), 2.67 (s, 3H), 1.43 (s, 9H). m/z (ES⁺), [M+H]⁺ 682.0. HPLC (A05) t_{R}=2.59 min.

### Step 5: 3-chloro-4-methoxy-N-[5-[5-(2-methoxyethoxy)-1H-benzimidazol-2-yl]-1-[(4-methoxyphenyl)methyl]pyrazol-3-yl]benzamide

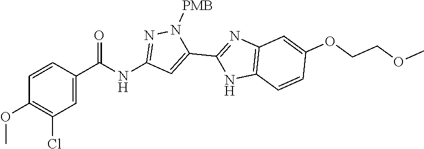

AcOH (4 mL) was added to tert-butyl N-[2-[[5-[(3-chloro-4-methoxy-benzoyl)amino]-2-[(4-methoxyphenyl)methyl]pyrazole-3-carbonyl]amino]-5-(2-methoxyethoxy)phenyl] carbamate (94 mg, 0.138 mmol), and the solution was refluxed for 18 h. The solution was concentrated. EtOAc (50 mL) was added, and the organic phase was washed with water (2×40 mL), sat. NaHCO₃ (30 mL), brine (40 mL), dried (Na₂SO₄), filtered, and concentrated to provide the title compound as a solid (48.1 mg, 62%). ¹H NMR (400 MHz, DMSO-d6) δ 12.97 (s, 1H), 11.05 (s, 1H), 8.15 (d, J=2.2 Hz, 1H), 8.06 (dd, J=8.7, 2.2 Hz, 1H), 7.50 (s, 1H), 7.24 (dd, J=11.8, 8.8 Hz, 4H), 6.95-6.83 (m, 4H), 6.01 (s, 2H), 4.14 (d, J=3.1 Hz, 2H), 3.94 (s, 3H), 3.72-3.69 (m, 2H), 3.69 (s, 3H), 3.33 (s, 3H). m/z (ES⁺), [M+H]⁺ 563.5. HPLC (A05) t_{R}=2.46 min.

### Step 6: 3-chloro-4-methoxy-N-[5-[5-(2-methoxyethoxy)-1H-benzimidazol-2-yl]-1H-pyrazol-3-yl]benzamide

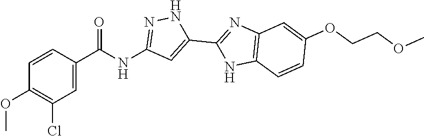 [SMILES: COCCOc1ccc2[nH]c(-c3cc(NC(=O)c4ccc(OC)c(Cl)c4)n[nH]3)nc2c1]

TFA (2 mL) was added to 3-chloro-4-methoxy-N-[5-[5-(2-methoxyethoxy)-1H-benzimidazol-2-yl]-1-[(4-methoxyphenyl)methyl]pyrazol-3-yl]benzamide (48.1 mg, 0.0856 mmol). The mixture was stirred at 23° C. for 18 h. The mixture was concentrated, and the residue was purified by preparative HPLC purification (BEH C18 30 ×150) eluting with water (10 mM (NH₄)(HCO₃)) and MeCN (42-52%) to provide the title compound as a solid (22.7 mg, 60%) in a 1:1 mixture of tautomers. ¹H NMR (400 MHz, DMSO-d6) δ 13.41 (s, 1H), 12.88 (s, 1H), 10.98 (s, 1H), 8.15 (d, J=2.1 Hz, 1H), 8.06 (dd, J=8.5, 2.0 Hz, 1H), 7.53 [6.61] (s, 1H), 7.45-7.32 (m, 1H), 7.29 (d, J=7.7 Hz, 1H), 7.21-7.12 [7.06-6.94] (m, 1H), 6.92-6.78 (m, 1H), 4.15-4.12 (s, 2H), 3.95 (s, 3H), 3.71-3.67 (m, 2H), 3.33 (s, 3H). m/z (ES⁺), [M+H]⁺ 442.9. HPLC (A05) t_{R}=2.17 min.

### EXAMPLE B72

### Preparation of Compound A-257

### Step 1: N-[5-(1H-benzimidazol-2-yl)-1-methyl-pyrazol-3-yl]-4-piperazin-1-yl-benzamide

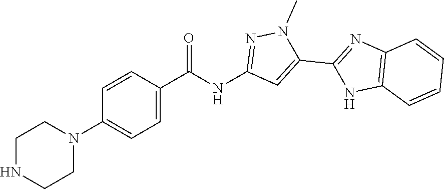 [SMILES: Cn1nc(NC(=O)c2ccc(N3CCNCC3)cc2)cc1-c1nc2ccccc2[nH]1]

Piperazine (1541 mg, 17.9 mmol) was added to a solution of N-[5-(1H-benzimidazol-2-yl)-1-methyl-pyrazol-3-yl]-4-fluoro-benzamide (300 mg, 0.895 mmol) in DMSO (1 mL). The mixture was heated to 110° C. and stirred for 18 h. EtOAc (100 mL) was added, and the organic phase was washed with water (3×50 mL), brine (50 mL), dried (Na₂SO₄), filtered, and concentrated. The residue was purified by silica gel chromatography (25 g cartridge) eluting with DCM and MeOH (0-80%) to provide the title compound as a solid (281 mg, 78%). ¹H NMR (400 MHz, DMSO-d6) δ 12.99 (s, 1H), 10.64 (s, 1H), 7.89 (d, J=9.0 Hz, 2H), 7.73-7.45 (m, 2H), 7.41 (s, 1H), 7.18 (dd, J=6.4, 3.0 Hz, 2H), 6.91 (d, J=9.0 Hz, 2H), 4.24 (s, 3H), 3.14 (dd, J=5.9, 4.1 Hz, 4H), 2.80-2.73 (m, 4H). m/z (ES⁺), [M+H]⁺ 402.2. HPLC (A05) t_{R}=1.87 min.

### Step 2: N-[5-(1H-benzimidazol-2-yl)-1-methyl-pyrazol-3-yl]-4-[4-(2-hydroxypropyl)piperazin-1-yl]benzamide

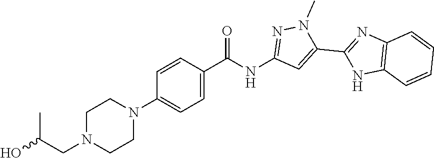 [SMILES: CC(O)CN1CCN(c2ccc(C(=O)Nc3cc(-c4nc5ccccc5[nH]4)n(C)n3)cc2)CC1]

1-bromopropan-2-ol (0.0277 g, 0.199 mmol) was added to a solution of N-[5-(1H-benzimidazol-2-yl)-1-methyl-pyrazol-3-yl]-4-piperazin-1-yl-benzamide (80 mg, 0.199 mmol) in DMF (2 mL). The solution was stirred at 23° C. for 1 h, then heated to 80° C. for 17 h. The mixture was concentrated and EtOAc (100 mL) was added. The organic phase was washed with water (3×50 mL), brine (50 mL), dried (Na₂SO₄), filtered, and concentrated. The residue was purified by silica gel chromatography (12 g cartridge) eluting with DCM and MeOH (0-10%) to provide the title compound as a solid (32.6 mg, 36%). ¹H NMR (400 MHz, DMSO-d6) δ 13.00 (s, 1H), 10.66 (s, 1H), 7.90 (d, J=9.0 Hz, 2H), 7.66 (d, J=6.8 Hz, 1H), 7.48 (d, J=7.2 Hz, 1H), 7.42 (s, 1H), 7.20 (p, J=6.3 Hz, 2H), 6.93 (d, J=9.1 Hz, 2H), 4.28 (d, J=4.1 Hz, 1H), 4.25 (s, 3H), 3.76 (dp, J=12.3, 6.1 Hz, 1H), 3.25-3.20 (m, 4H), 2.54-2.47 (m, 4H), 2.21 (ddd, J=17.7, 12.4, 6.2 Hz, 2H), 1.02 (d, J=6.2 Hz, 3H). m/z (ES⁺), [M+H]⁺ 460.4. HPLC (A05) t_{R}=1.91 min.

### EXAMPLE B73

### Preparation of Compound A-257

### Step 1: methyl 2-[4-[4-[[5-(1H-benzimidazol-2-yl)-1-methyl-pyrazol-3-yl]carbamoyl]phenyl]piperazin-1-yl]acetate

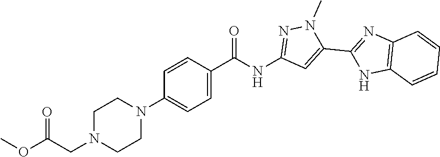 [SMILES: COC(=O)CN1CCN(c2ccc(C(=O)Nc3cc(-c4nc5ccccc5[nH]4)n(C)n3)cc2)CC1]

Methyl 2-bromoacetate (0.0283 mL, 0.299 mmol) was added to a solution of N-[5-(1H-benzimidazol-2-yl)-1-methyl-pyrazol-3-yl]-4-piperazin-1-yl-benzamide (Compound A-261) (120 mg, 0.299 mmol) in DMF (3 mL), and mixture was stirred at 23° C. for 120 h. The mixture was concentrated, and the residue was purified by silica gel chromatography (12 g cartridge) eluting with DCM and MeOH (0-10%) to provide the title compound as a solid (57 mg, 40%). ¹H NMR (400 MHz, DMSO-d6) δ 13.05 (s, 1H), 10.71 (s, 1H), 7.95 (d, J=9.1 Hz, 2H), 7.76-7.64 (m, 1H), 7.59-7.50 (m, 1H), 7.47 (s, 1H), 7.29-7.17 (m, 2H), 6.99 (d, J=9.0 Hz, 2H), 4.30 (s, 3H), 3.64 (s, 3H), 3.31-3.25 (m, 6H), 2.67-2.63 (m, 4H). m/z (ES⁺), [M+H]⁺ 474.3. HPLC (A05) t_{R}=2.12 min.

### Step 2: N-[5-(1H-benzimidazol-2-yl)-1-methyl-pyrazol-3-yl]-4-[4-(2-hydroxy-2-methyl-propyl)piperazin-1-yl]benzamide

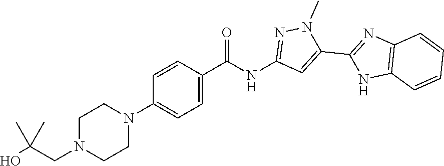 [SMILES: Cn1nc(NC(=O)c2ccc(N3CCN(CC(C)(C)O)CC3)cc2)cc1-c1nc2ccccc2[nH]1]

A solution of MeMgBr in ether (3.00 mol/L, 0.197 mL, 0.591 mmol) was added dropwise to a solution of methyl 2-[4-[4-[[5-(1H-benzimidazol-2-yl)-1-methyl-pyrazol-3-yl]carbamoyl]-phenyl]piperazin-1-yl]acetate (56 mg, 0.118 mmol) in Me-THF (3 mL). The mixture was stirred at 23° C. for 2 h. Additional MeMgBr in ether (3.00 mol/L, 0.197 mL, 0.591 mmol) was added, and the mixture was stirred at 23° C. for 18 h. Water (50 mL) was added, and the aq. phase was extracted with EtOAc (3×40 mL). The combined organic phases were washed with brine (30 mL), dried (Na₂SO₄), filtered, and concentrated. The residue was purified by silica gel chromatography (12 g cartridge) eluting with DCM and MeOH (0-10%) and by preparative HPLC (BEH C18 30 ×150) eluting with water (10 mM (NH₄)(HCO₂)) and MeCN (22-32%) to provide the formate salt of the title compound as a solid (25.7 mg, 42%). ¹H NMR (400 MHz, DMSO-d6) δ 10.65 (s, 1H), 8.35 (s, 1H), 7.90 (d, J=9.0 Hz, 2H), 7.74-7.45 (m, 2H), 7.42 (s, 1H), 7.23-7.15 (m, 2H), 6.92 (d, J=9.1 Hz, 2H), 4.25 (s, 3H), 3.24-3.21 (m, 4H), 2.66-2.57 (m, 4H), 2.20 (s, 2H), 1.07 (s, 6H). m/z (ES⁺), [M−HCO₂]⁺ 474.3. HPLC (A05) t_{R}=1.90 min.

### EXAMPLE B74

### Preparation of Compound A-260

### Step 1: 2-(trideuteriomethoxy)acetaldehyde

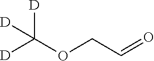

DMSO (0.198 mL, 2.78 mmol) was added dropwise to a mixture of oxalyl chloride (0.119 mL, 1.39 mmol) in DCM (3.60 mL) at −78° C. The mixture was stirred at −78° C. for 2 min. 2-(trideuteriomethoxy)ethanol (100 mg, 1.26 mmol) in DCM (3.24 mL) was added, and the mixture was stirred at −78° C. for 1 h. NEt₃ (0.529 mL, 3.79 mmol) was added, and the mixture was stirred at 23° C. for 90 min to form the aldehyde in solution, which was used in the next step without purification.

### Step 1: N-[5-(1H-benzimidazol-2-yl)-1-methyl-pyrazol-3-yl]-6-[4-[2-(trideuterio-methoxy)ethyl]piperazin-1-yl]pyridine-3-carboxamide

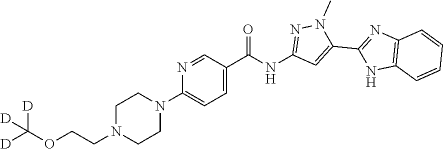 [SMILES: [2H]C([2H])([2H])OCCN1CCN(c2ccc(C(=O)Nc3cc(-c4nc5ccccc5[nH]4)n(C)n3)cn2)CC1]

2-(trideuteriomethoxy)acetaldehyde (in solution from step 1, 97.5 mg, 1.26 mmol) was added to a solution of N-[5-(1H-benzimidazol-2-yl)-1-methyl-pyrazol-3-yl]-6-piperazin-1-yl-pyridine-3-carboxamide (Compound A-216) (100 mg, 0.248 mmol) in DCM (2.4 mL) at 23° C. Na₂SO₄ was added. The mixture was stirred at 23° C. for 18 h, NaBH₃CN (31.2 mg, 0.497 mmol) was slowly added at 0° C. The mixture was stirred at 23° C. for 6 h. Water (10 mL) was added. The aq. phase was extracted with EtOAc (2×20 mL), and the combined organic phases were washed with brine (10 mL), dried (Na₂SO₄), filtered, and concentrated. The residue was purified by silica gel chromatography (25 g cartridge) eluting with DCM and MeOH (0-20%) and by preparative HPLC (BEH, C18) eluting with water [10 mM (NH₄)(HCO₂)] and MeCN (22-32%). The necessary fractions were concentrated. The residue was dissolved in DMSO (0.500 mL). NEt₃ (0.500 mL) was added, and the mixture was stirred at 23° C. for 5 min. Water (10 mL) was added. The precipitate was filtered, washed with water (10 mL), and dried to provide the title compound as a white solid (22 mg, 19%). ¹H NMR (400 MHz, DMSO-d6) δ 13.05 (s, 1H), 10.83 (s, 1H), 8.79 (d, J=2.4 Hz, 1H), 8.14 (dd, J=9.1, 2.5 Hz, 1H), 7.79-7.64 (m, 1H), 7.61-7.50 (m, 1H), 7.47 (s, 1H), 7.29-7.19 (m, 2H), 6.88 (d, J=9.1 Hz, 1H), 4.30 (s, 3H), 3.65-3.59 (m, 4H), 3.47 (t, J=5.8 Hz, 2H), 2.55-2.50 (m, 6H). m/z (ES⁺), [M+H]⁺ 464.3; HPLC (A05) t_{R}=2.37 min.

### EXAMPLE B75

### Preparation of Compound A-261

### Step 1: tert-butyl N-(5-fluoro-2-nitro-phenyl)carbamate and tert-butyl N-tert-butoxycarbonyl-N-(5-fluoro-2-nitro-phenyl)carbamate

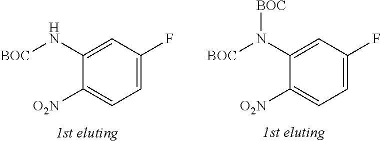 [SMILES: CC(C)(C)OC(=O)Nc1cc(F)ccc1[N+](=O)[O-] ; CC(C)(C)OC(=O)N(C(=O)OC(C)(C)C)c1cc(F)ccc1[N+](=O)[O-]]

A solution of Boc₂O (4.4 g, 20.2 mmol) in 1,4-dioxane (20 mL) was added dropwise to a solution of 5-fluoro-2-nitro-aniline (3 g, 19.2 mmol), DMAP (0.235 g, 1.92 mmol) and NEt₃ (4.02 mL, 28.8 mmol) in 1,4-Dioxane (60 mL). The mixture was stirred at 23° C. for 18 h. Boc₂O (1.89 g, 8.6 mmol), DMAP (0.117 g, 0.960 mmol) and NEt₃ (2.68 mL, 19.2 mmol) were added. The mixture was stirred at 23° C. for 24 h. The mixture was concentrated, and the residue was purified by silica gel chromatography (120 g cartridge) eluting with hexanes and EtOAc (0-60%) to provide two compounds as solids: First eluting: tert-butyl N-(5-fluoro-2-nitro-phenyl)carbamate (1.02 g, 21%) : ¹H NMR (300 MHz, DMSO-d6) δ 9.74 (s, 1H), 8.13 (dd, J=9.3, 5.9 Hz, 1H), 7.68 (dd, J=10.9, 2.8 Hz, 1H), 7.19-7.07 (m, 1H), 1.46 (s, 9H). HPLC (A05) t_{R}=2.82 min. Second eluting: tert-butyl N-tert-butoxycarbonyl-N-(5-fluoro-2-nitro-phenyl)carbamate phenyl)carbamate (2.40 g, 35%): ¹H NMR (400 MHz, DMSO-d6) δ 8.25 (dd, J=9.2, 5.5 Hz, 1H), 7.73 (dd, J=9.1, 2.8 Hz, 1H), 7.54 (ddd, J=9.1, 7.7, 2.8 Hz, 1H), 1.33 (s, 18H). HPLC (A05) t_{R}=2.78 min.

### Step 2: tert-butyl N-[5-(4-methylpiperazin-1-yl)-2-nitro-phenyl]carbamate

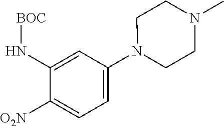 [SMILES: CN1CCN(c2ccc([N+](=O)[O-])c(NC(=O)OC(C)(C)C)c2)CC1]

1-Methylpiperazine (0.121 mL, 1.09 mmol) was added to a solution of tert-butyl N-(5-fluoro-2-nitro-phenyl)carbamate (85% pure, 300 mg, 0.995 mmol) and DIEA (0.341 mL, 1.99 mmol) in DMSO (6.63 mL). The mixture was stirred at 110° C. for 3 h. EtOAc (40 mL) was added, and the organic phase was washed with water (3×40 mL), brine (50 mL), dried (Na₂SO₄), filtered, and concentrated. The residue was purified by silica gel chromatography (24 g cartridge) eluting with EtOAc and hexanes (0-100%), then with DCM and MeOH (0-10%) to provide the title compound as a solid (325 mg, 97%). ¹H NMR (400 MHz, DMSO-d6) δ 10.03 (s, 1H), 8.01 (d, J=9.7 Hz, 1H), 7.68 (d, J=2.8 Hz, 1H), 6.77 (dd, J=9.7, 2.8 Hz, 1H), 3.45-3.41 (m, 4H), 2.45-2.40 (m, 4H), 2.22 (s, 3H), 1.48 (s, 9H). m/z (ES⁺), [M+H]⁺ 337.3. HPLC (A05) t_{R}=2.16 min.

### Step 3: tert-butyl N-[2-amino-5-(4-methylpiperazin-1-yl)phenyl]carbamate

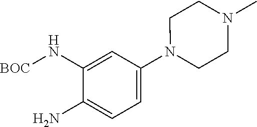 [SMILES: CN1CCN(c2ccc(N)c(NC(=O)OC(C)(C)C)c2)CC1]

Tert-butyl N-[5-(4-methylpiperazin-1-yl)-2-nitro-phenyl]carbamate (325 mg, 0.966 mmol) was dissolved in MeOH (50 mL) and transferred in a flask purged with nitrogen containing Pd/C (10.0%, 161 mg, 0.151 mmol). The mixture was stirred at 23° C. for 18 h under an atmosphere of hydrogen. The flask was purged with nitrogen, the solution was filtered on celite, and the filtrate was concentrated to provide the title compound as a foam (285 mg, 96%). ¹H NMR (300 MHz, DMSO-d6) δ 8.22 (s, 1H), 6.91 (d, J=2.0 Hz, 1H), 6.59 (d, J=8.6 Hz, 1H), 6.50 (dd, J=8.6, 2.6 Hz, 1H), 4.39 (s, 2H), 2.95-2.84 (m, 4H), 2.42 (d, J=4.9 Hz, 4H), 2.20 (s, 3H), 1.45 (s, 9H). m/z (ES⁺), [M+H]⁺ 307.2. HPLC (A05) t_{R}=1.75 min.

### Step 4

### tert-butyl N-[2-[[5-[(3-chloro-4-methoxy-benzoyl)amino]-2-[(4-methoxyphenyl) methyl]pyrazole-3-carbonyl]amino]-5-(4-methylpiperazin-1-yl)phenyl]carbamate

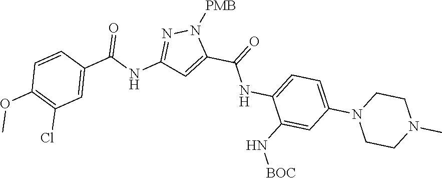

5-[(3-chloro-4-methoxy-benzoyl)amino]-2-[(4-methoxyphenyl)methyl]pyrazole-3-carboxylic acid (210 mg, 0.506 mmol) and HATU (192 mg, 0.506 mmol) were dissolved in DMF (2 mL) at 0° C. DIEA (0.173 mL, 1.01 mmol) was added, and the mixture was stirred at 0° C. for 5 min. Tert-butyl N-[2-amino-5-(4-methylpiperazin-1-yl)phenyl]carbamate (155 mg, 0.506 mmol) in DMF (1.78 mL) was added. The mixture was stirred at 23° C. for 18 h. The mixture was concentrated, and the residue was purified by silica gel chromatography (12 g cartridge) eluting with DCM and MeOH (0-15%) to provide the title compound as a solid (320 mg, 90%). ¹H NMR (300 MHz, DMSO-d6) δ 11.04 (s, 1H), 9.70 (s, 1H), 8.68-8.63 (m, 1H), 8.57 (s, 1H), 8.44 (d, J=8.4 Hz, 1H), 8.12 (d, J=2.2 Hz, 1H), 8.03 (dd, J=8.6, 2.2 Hz, 1H), 7.46-7.39 (m, 1H), 7.29-7.22 (m, 1H), 7.22-7.18 (m, 2H), 6.86 (d, J=8.7 Hz, 2H), 6.75 (dd, J=8.9, 2.7 Hz, 1H), 5.59 (s, 2H), 3.92 (s, 3H), 3.69 (s, 3H), 3.22-3.16 (m, 4H), 2.80-2.71 (m, 4H), 2.43 (s, 3H), 1.43 (s, 9H). m/z (ES⁺), [M+H]⁺ 705.2. HPLC (A05) t_{R}=2.39 min.

### Step 5: N-[2-amino-4-(4-methylpiperazin-1-yl)phenyl]-5-[(3-chloro-4-methoxy-benzoyl)amino]-2-[(4-methoxyphenyl)methyl]pyrazole-3-carboxamide

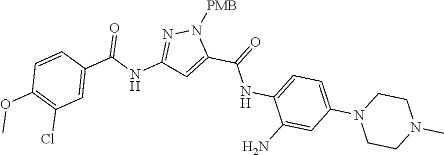

A solution of tert-butyl N-[2-[[5-[(3-chloro-4-methoxy-benzoyl)amino]-2-[(4-methoxyphenyl)-methyl]pyrazole-3-carbonyl]amino]-5-(4-methylpiperazin-1-yl)phenyl]carbamate (356 mg, 0.506 mmol) in HFIP (10 mL) was stirred at 100° C. for 4 h. The mixture was concentrated. The residue was purified by silica gel chromatography (25 g cartridge) eluting with DCM and MeOH (0-20%) to provide the title compound as a solid (120 mg, 40%). ¹H NMR (400 MHz, DMSO-d6) δ 11.00 (s, 1H), 9.65 (s, 1H), 8.14 (d, J=2.2 Hz, 1H), 8.05 (dd, J=8.7, 2.2 Hz, 1H), 7.50 (s, 1H), 7.26 (d, J=8.9 Hz, 1H), 7.21 (d, J=8.8 Hz, 2H), 6.95 (d, J=8.7 Hz, 1H), 6.89-6.85 (m, 2H), 6.35 (d, J=2.6 Hz, 1H), 6.23 (dd, J=8.7, 2.6 Hz, 1H), 5.60 (s, 2H), 4.74 (s, 2H), 3.93 (s, 3H), 3.71 (s, 3H), 3.07 (m, 4H), 2.51 (d, J=1.8 Hz, 4H), 2.27 (s, 3H). m/z (ES⁺) [M+H]⁺ 604.1. HPLC (A05) t_{R}=2.07 min.

### Step 6: 3-chloro-4-methoxy-N-[1-[(4-methoxyphenyl)methyl]-5-[5-(4-methylpiperazin-1-yl)-1H-benzimidazol-2-yl]pyrazol-3-yl]benzamide

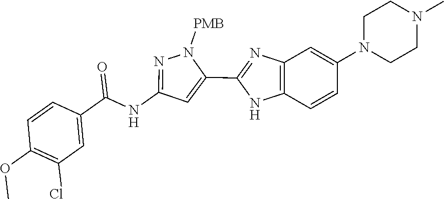

A solution of tert-butyl N-[2-[[5-[(3-chloro-4-methoxy-benzoyl)amino]-2-[(4-methoxyphenyl)-methyl]pyrazole-3-carbonyl]amino]-5-(4-methylpiperazin-1-yl)phenyl]carbamate (100 mg, 0.166 mmol) in AcOH (3.50 mL) was stirred at 65° C. for 18 h. The mixture was concentrated. The residue was purified by silica gel chromatography (12 g cartridge) eluting with DCM and MeOH (0-15%) and by preparative HPLC (BEH, C18) eluting with water [10 mM (NH₄)(HCO₃)] and MeCN (48-58%) to provide the title compound as a solid (22 mg, 23%). ¹H NMR (300 MHz, DMSO-d6) δ 12.80 (s, 1H), 11.04 (s, 1H), 8.15 (d, J=2.1 Hz, 1H), 8.06 (dd, J=8.7, 2.2 Hz, 1H), 7.57-7.50 (m, 1H), 7.46 (s, 1H), 7.29-7.19 (m, 3H), 7.19-7.10 (m, 1H), 7.03-6.94 (m, 1H), 6.87-6.81 (m, 2H), 6.00 (s, 2H), 3.94 (s, 3H), 3.69 (s, 3H), 3.18-3.10 (m, 4H), 2.55-2.51 (m, 4H), 2.24 (s, 3H). m/z (ES⁺), [M+H]⁺ 586.2. HPLC (A05) t_{R}=2.12 min.

### Step 7: 3-chloro-4-methoxy-N-[5-[5-(4-methylpiperazin-1-yl)-1H-benzimidazol-2-yl]-1H-pyrazol-3-yl]benzamide

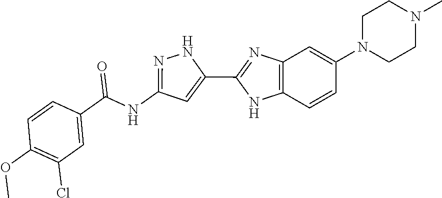 [SMILES: COc1ccc(C(=O)Nc2cc(-c3nc4cc(N5CCN(C)CC5)ccc4[nH]3)[nH]n2)cc1Cl]

TFA (0.785 mL) was added to 3-chloro-4-methoxy-N-[1-[(4-methoxyphenyl)methyl]-5-[5-(4-methylpiperazin-1-yl)-1H-benzimidazol-2-yl]pyrazol-3-yl]benzamide (36 mg, 0.0307 mmol). The mixture was stirred at 23° C. for 1 h and concentrated. The residue was dissolved in DMSO (0.500 mL). NEt₃ (0.500 mL) was added, and the mixture was stirred at 23° C. for 5 min. Water (10 mL) was added. The mixture was filtered. The solid was washed with water (10 mL) and dried under high vacuum to provide the title compound a solid in a 1:1 mixture of tautomers (14 mg, 98%). ¹H NMR (400 MHz, DMSO-d6) δ 13.50-13.30 [13.25-13.00] (m, 1H), 12.80-12.60 [12.50-12.30] (m, 1H), 10.90-11.00 [11.15-11.00] (m, 1H), 8.15 (s, 1H), 8.06 (d, J=7.8 Hz, 1H), 7.49 [7.33] (d, J=8.8 Hz, 1H), 7.39-7.25 (m, 1H), 7.14-7.10 [6.72-6.54] (m, 1H), 7.03-6.94 (m,1H), 6.94-6.90 (m, 1H), 3.95 (s, 3H), 3.22-3.11 (m, 4H), 2.71-2.56 (m, 4H), 2.39-2.30 (m, 3H). m/z (ES⁺), [M+H]⁺ 466.3; HPLC (A05) t_{R}=2.45 min.

### EXAMPLE B76

### Preparation of Compound A-262

### Step 1: N-[5-(1H-benzimidazol-2-yl)-1-methyl-pyrazol-3-yl]-6-[2-(hydroxy-methyl)morpholin-4-yl]-2-methyl-pyridine-3-carboxamide

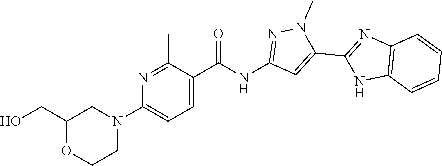 [SMILES: Cc1nc(N2CCOC(CO)C2)ccc1C(=O)Nc1cc(-c2nc3ccccc3[nH]2)n(C)n1]

Morpholin-2-ylmethanol (44.7 mg, 0.382 mmol) was added to a solution of N-[5-(1H-benzimidazol-2-yl)-1-methyl-pyrazol-3-yl]-6-chloro-2-methyl-pyridine-3-carboxamide (Compound A-236) (70 mg, 0.191 mmol) and DIEA (0.0653 mL, 0.382 mmol) in DMSO (1.50 mL). The mixture was heated to 110° C. and stirred for 18 h. EtOAc (100 mL) was added, and the organic phase was washed with water (3×50 mL), brine (50 mL), dried (Na₂SO₄), filtered, and concentrated. The residue was purified by silica gel chromatography (12 g cartridge) eluting with DCM and MeOH (0-20%) to provide the title compound as a solid (53 mg, 62%). 1H NMR (400 MHz, DMSO-d6) δ 13.05 (s, 1H), 10.75 (s, 1H), 7.77 (d, J=8.8 Hz, 1H), 7.74-7.65 (m, 1H), 7.53 (dd, J=17.1, 6.8 Hz, 1H), 7.47 (s, 1H), 7.29-7.19 (m, 2H), 6.69 (d, J=8.8 Hz, 1H), 4.80 (t, J=5.6 Hz, 1H), 4.35-4.26 (m, 4H), 4.15 (d, J=13.8 Hz, 1H), 3.98-3.92 (m, 1H), 3.58-3.41 (m, 4H), 2.88 (td, J=12.5, 3.3 Hz, 1H), 2.62 (dd, J=13.0, 10.1 Hz, 1H), 2.52 (s, 3H). m/z (ES⁺), [M+H]⁺ 448.3. HPLC (A05) t_{R}=2.02 min.

### EXAMPLE B77

### Preparation of Compound A-263

### Step 1: N-[5-(1H-benzimidazol-2-yl)-1-methyl-pyrazol-3-yl]-6-[3-(hydroxy-methyl)-4-methyl-piperazin-1-yl]pyridine-3-carboxamide

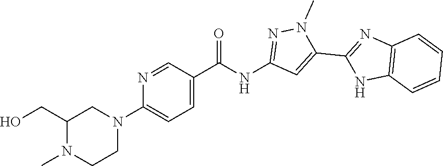 [SMILES: CN1CCN(c2ccc(C(=O)Nc3cc(-c4nc5ccccc5[nH]4)n(C)n3)cn2)CC1CO]

(1-methylpiperazin-2-yl)methanol dihydrochloride (40.3 mg, 0.198 mmol) was added to a solution of N-[5-(1H-benzimidazol-2-yl)-1-methyl-pyrazol-3-yl]-6-chloro-pyridine-3-carboxamide (70 mg, 0.198 mmol) and DIEA (0.136 mL, 0.794 mmol) in DMSO (1 mL). The mixture was heated to 110° C. and stirred for 18 h. Water (50 mL) was added, and the aq. phase was extracted with EtOAc (3×40 mL). The combined organic phases were washed with brine (40 mL), dried (Na₂SO₄), filtered, and concentrated. The residue was purified by silica gel chromatography (12 g cartridge) eluting with DCM (1% NEt₃) and MeOH (0-15%) and by preparative HPLC (BEH C18 30×150) eluting with water (10 mM (NH₄)(HCO₂)) and MeCN (15-25%) to provide the formate salt of the title compound as a solid. Water (3 mL) was added to the salt. Sat. aq. NaHCO₃ was added dropwise until a precipitate formed. EtOAc (20 mL) was added. The organic phase was washed with water (3×10 mL) and concentrated to provide the title compound as a solid (36.7 mg, 41%). ¹H NMR (400 MHz, DMSO-d6) δ 13.08 (s, 1H), 10.95 (s, 1H), 9.77 [5.69] (t, J=4.5 Hz, 1H), 8.86 (d, J=2.4 Hz, 1H), 8.26 (dd, J=9.0, 2.3 Hz, 1H), 7.73 (d, J=7.3 Hz, 1H), 7.55 (d, J=7.2 Hz, 1H), 7.49 (s, 1H), 7.32-7.18 (m, 2H), 7.07 (d, J=9.1 Hz, 1H), 4.68-4.49 (m, 2H), 4.32 (s, 3H), 4.00-3.83 (m, 1H), 3.80-3.68 (m, 1H), 3.46 (d, J=11.6 Hz, 1H), 3.31-3.15 (m, 4H), 2.89 [2.88] (s, 3H). ¹H NMR (500 MHz, MeOD-d4) δ 8.81 (d, J=2.0 Hz, 1H), 8.18 (dd, J=9.0, 2.5 Hz, 1H), 7.79-7.48 (m, 2H), 7.31 (dd, J=5.7, 2.4 Hz, 2H), 7.28 (s, 1H), 7.02 (d, J=9.1 Hz, 1H), 4.68-4.53 (m, 2H), 4.25 (s, 3H), 4.09-3.99 (m, 1H), 3.80 (dd, J=12.4, 2.3 Hz, 1H), 3.54-3.46 (m, 1H), 3.42-3.33 (m, 2H), 3.29-3.18 (m, 2H), 2.95 (s, 3H). m/z (ES⁺), [M+H]⁺ 447.2. HPLC (A05) t_{R}=1.85 min.

### EXAMPLE B78

### Preparation of Compound A-264

### Step 1: N-[5-(1H-benzimidazol-2-yl)-1-methyl-pyrazol-3-yl]-2-methyl-6-[4-(oxetan-3-yl)piperazin-1-yl]pyridine-3-carboxamide

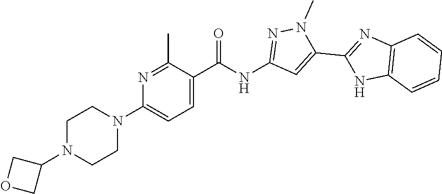 [SMILES: Cc1nc(N2CCN(C3COC3)CC2)ccc1C(=O)Nc1cc(-c2nc3ccccc3[nH]2)n(C)n1]

1-(oxetan-3-yl)piperazine (54.3 mg, 0.382 mmol) was added to a solution of N-[5-(1H-benzimidazol-2-yl)-1-methyl-pyrazol-3-yl]-6-chloro-2-methyl-pyridine-3-carboxamide (Compound A-236) (70 mg, 0.191 mmol) in DMSO (1.50 mL). The mixture was heated to 110° C. and stirred for 18 h. Water (20 mL) was added, and the precipitate was filtered. The residue was purified by silica gel chromatography (12 g cartridge) eluting with DCM and MeOH (0-15%). The solid was further purified by preparative HPLC (BEH C18 30 ×150) eluting with water (10 mM (NH₄)(HCO₃)) and MeCN (28-38%) to provide the title compound as a solid (33.5 mg, 37%). ¹H NMR (400 MHz, DMSO-d6) δ 13.04 (s, 1H), 10.73 (s, 1H), 7.75 (d, J=8.8 Hz, 1H), 7.73-7.63 (m, 1H), 7.61-7.49 (m, 1H), 7.48 (s, 1H), 7.30-7.18 (m, 2H), 6.70 (d, J=8.8 Hz, 1H), 4.57 (t, J=6.5 Hz, 2H), 4.48 (t, J=6.1 Hz, 2H), 4.29 (s, 3H), 3.64-3.55 (m, 4H), 3.43 (p, J=6.2 Hz, 1H), 2.51 (s, 3H), 2.37-2.31 (m, 4H). m/z (ES⁺), [M+H]⁺ 473.8. HPLC (A05) t_{R}=1.99 min.

### EXAMPLE B79

### Preparation of Compound A-265

### Step 1: tert-butyl N-(5-morpholino-2-nitro-phenyl)carbamate

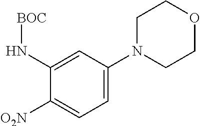 [SMILES: CC(C)(C)OC(=O)Nc1cc(N2CCOCC2)ccc1[N+](=O)[O-]]

Morpholine (0.0958 mL, 1.09 mmol) was added to a solution of tert-butyl N-(5-fluoro-2-nitro-phenyl)carbamate (Compound A-266) (85% pure, 300 mg, 0.995 mmol) and DIEA (0.341 mL, 1.99 mmol) in DMSO (6.63 mL). The mixture was stirred at 110° C. for 3 h. EtOAc (40 mL) was added. The organic phase was washed with water (3×40 mL), brine (50 mL), dried (Na₂SO₄), filtered, and concentrated. The residue was purified by silica gel chromatography (24 g cartridge) eluting with EtOAc and hexanes (0-100%) to provide the title compound as a solid (288 mg, 90%). ¹H NMR (300 MHz, DMSO-d6) δ 10.00 (s, 1H), 8.03 (d, J=9.7 Hz, 1H), 7.68 (d, J=2.7 Hz, 1H), 6.78 (dd, J=9.7, 2.7 Hz, 1H), 3.77-3.69 (m, 4H), 3.43-3.36 (m, 4H), 1.48 (s, 9H). m/z (ES⁺), [M+H]⁺ 324.2. HPLC (A05) t_{R}=2.74 min.

### Step 2: tert-butyl N-(2-amino-5-morpholino-phenyl)carbamate

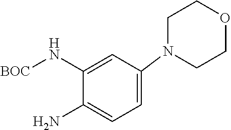 [SMILES: CC(C)(C)OC(=O)Nc1cc(N2CCOCC2)ccc1N]

Tert-butyl N-(5-morpholino-2-nitro-phenyl)carbamate (288 mg, 0.891 mmol) was dissolved in MeOH (50 mL) and transferred in a flask purged with nitrogen containing Pd/C (10.0%, 148 mg, 0.139 mmol). The mixture was stirred at 23° C. for 18 h under an atmosphere of hydrogen. The flask was purged with nitrogen, the solution was filtered on celite, washed with MeOH (40 mL), and the filtrate was concentrated to provide the title compound as a foam (230 mg, 88%). ¹H NMR (300 MHz, DMSO-d6) δ 8.24 (s, 1H), 6.93 (s, 1H), 6.61 (d, J=8.6 Hz, 1H), 6.52 (dd, J=8.6, 2.7 Hz, 1H), 4.42 (s, 2H), 3.73-3.66 (m, 4H), 2.90-2.84 (m, 4H), 1.45 (s, 9H). m/z (ES⁺), [M+H]⁺ 294.2. HPLC (A05) t_{R}=2.09 min.

### Step 3: tert-butyl N-[2-[[5-[(3-chloro-4-methoxy-benzoyl)amino]-2-[(4-methoxyphenyl) methyl]pyrazole-3-carbonyl]amino]-5-morpholino-phenyl]carbamate

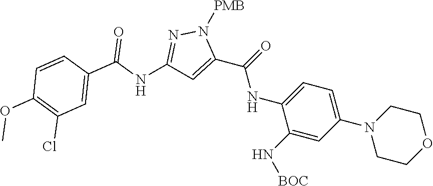

5-[(3-chloro-4-methoxy-benzoyl)amino]-2-[(4-methoxyphenyl)methyl]pyrazole-3-carboxylic acid (163 mg, 0.392 mmol) and HATU (149 mg, 0.392 mmol) were dissolved in DMF (1.93 mL) at 0° C. DIEA (0.134 mL, 0.784 mmol) was added, and the mixture was stirred at 0° C. for 5 min. Tert-butyl N-(2-amino-5-morpholino-phenyl)carbamate (115 mg, 0.392 mmol) in DMF (1.90 mL) was added. The mixture was stirred at 23° C. for 18 h and concentrated. The residue was purified by silica gel chromatography (12 g cartridge) eluting with DCM and MeOH (0-15%) to provide the title compound as a solid (230 mg, 85%). ¹H NMR (300 MHz, DMSO-d6) δ 11.06 (s, 1H), 9.72 (s, 1H), 8.58 (s, 1H), 8.14 (d, J=2.2 Hz, 1H), 8.05 (dd, J=8.7, 2.2 Hz, 1H), 7.49 (s, 1H), 7.30-7.19 (m, 5H), 6.87 (d, J=8.7 Hz, 2H), 6.75 (dd, J=8.9, 2.7 Hz, 1H), 5.61 (s, 2H), 3.94 (s, 3H), 3.78-3.73 (m, 4H), 3.71 (s, 3H), 3.11-3.05 (m, 4H), 1.44 (s, 9H). m/z (ES⁺), [M+H]⁺ 691.9. HPLC (A05) t_{R}=2.57 min.

### Step 4: N-(2-amino-4-morpholino-phenyl)-5-[(3-chloro-4-methoxy-benzoyl)amino]-2-[(4-methoxyphenyl)methyl]pyrazole-3-carboxamide

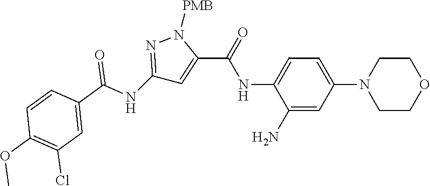

A solution of tert-butyl N-[2-[[5-[(3-chloro-4-methoxy-benzoyl)amino]-2-[(4-methoxyphenyl)-methyl]pyrazole-3-carbonyl]amino]-5-morpholino-phenyl]carbamate (230 mg, 0.333 mmol) in HFIP (8 mL) was stirred at 100° C. for 4 h. The mixture was concentrated. The residue was purified by silica gel chromatography (25 g cartridge) eluting with DCM and MeOH (0-20%) to provide the title compound as a solid (170 mg, 74%). ¹H NMR (500 MHz, DMSO-d6) δ 11.00 (s, 1H), 9.66 (s, 1H), 8.14 (d, J=2.2 Hz, 1H), 8.05 (dd, J=8.7, 2.2 Hz, 1H), 7.50 (s, 1H), 7.26 (d, J=8.9 Hz, 1H), 7.22 (d, J=8.8 Hz, 2H), 6.97 (d, J=8.7 Hz, 1H), 6.89-6.86 (m, 2H), 6.35 (d, J=2.6 Hz, 1H), 6.24 (dd, J=8.7, 2.6 Hz, 1H), 5.60 (s, 2H), 4.77 (s, 2H), 3.94 (s, 3H), 3.75-3.72 (m, 4H), 3.71 (s, 3H), 3.06-2.99 (m, 4H). m/z (ES⁺) [M+H]⁺ 592.3. HPLC (A05) t_{R}=2.34 min.

### Step 5: 3-chloro-4-methoxy-N-[1-[(4-methoxyphenyl)methyl]-5-(5-morpholino-1H-benzimidazol-2-yl)pyrazol-3-yl]benzamide

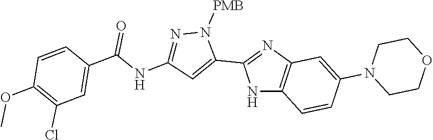

A solution of N-(2-amino-4-morpholino-phenyl)-5-[(3-chloro-4-methoxy-benzoyl)amino]-2-[(4-methoxyphenyl)methyl]pyrazole-3-carboxamide (170 mg, 0.288 mmol) in AcOH (5.95 mL) was stirred at 65° C. for 18 h. After cooling to 23° C., the mixture was concentrated. The residue was purified by silica gel chromatography (25 g cartridge) eluting with DCM and MeOH (0-15%) and by reverse phase chromatography (C18, 20 g cartridge) eluting with water [10 mM (NH₄)(HCO₂)] and MeCN (51-61%) to provide the title compound as a solid (156 mg, 95%). ¹H NMR (400 MHz, DMSO-d6) δ 11.02 (s, 1H), 8.41 (br, 1H), 8.15 (d, J=2.2 Hz, 1H), 8.06 (dd, J=8.7, 2.2 Hz, 1H), 7.53-7.49 (m, 1H), 7.48 (s, 1H), 7.26 (d, J=8.9 Hz, 1H), 7.24-7.21 (m, 2H), 7.01 (dd, J=8.7, 1.4 Hz, 2H), 6.87-6.83 (m, 2H), 6.01 (s, 2H), 3.94 (s, 3H), 3.83-3.74 (m, 4H), 3.69 (s, 3H), 3.14-3.08 (m, 4H). m/z (ES⁺), [M+H]⁺ 574.6. HPLC (A05) t_{R}=2.52 min.

### Step 6: 3-chloro-4-methoxy-N-[15-(5-morpholino-1H-benzimidazol-2-yl)-1H-pyrazol-3-yl]benzamide

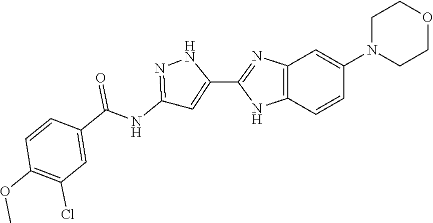 [SMILES: COc1ccc(C(=O)Nc2cc(-c3nc4cc(N5CCOCC5)ccc4[nH]3)[nH]n2)cc1Cl]

TFA (5 mL) was added to 3-chloro-4-methoxy-N-[1-[(4-methoxyphenyl)methyl]-5-(5-morpholino-1H-benzimidazol-2-yl)pyrazol-3-yl]benzamide (156 mg, 0.272 mmol). The mixture was stirred at 23° C. for 30 min and concentrated. The residue was dissolved in DMSO (0.5 mL). NEt₃ (0.5 mL) was added, and the mixture was stirred at 23° C. for 5 min. Water (10 mL) was added. The mixture was filtered. The solid was washed with water (10 mL) and dried under high vacuum to provide the title compound as a white solid (48.5 mg, 40%) in a 1:1 mixture of tautomers. ¹H NMR (400 MHz, DMSO-d6) δ 13.46-13.29 [13.45-13.38] [13.15-13.02] (m, 1H), 12.82-12.68 [12.82-12.76] [12.56-12.40] (m, 1H), 11.03-10.92 [11.20-11.05] (m, 1H), 8.15 (s, 1H), 8.06 (d, J=8.0 Hz, 1H), 7.55-7.45 [7.44-7.32] (m, 1H), 7.28 (d, J=7.7 Hz, 1H), 7.12 [6.67-6.56] (s, 1H), 7.05-6.95 (m, 1H), 6.92 (s, 1H), 3.95 (s, 3H), 3.81-3.71 (m, 4H), 3.18-3.07 (m, 4H). m/z (ES⁺), [M+H]⁺ 453.2; HPLC (A05) t_{R}=3.11 min.

### EXAMPLE B80

### Preparation of Compound A-266

### Step 1: 2-(cyclopropoxy)acetaldehyde

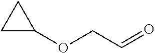

DMSO (0.153 mL, 2.15 mmol) was added dropwise to a mixture of oxalyl chloride (92.4 μL, 1.08 mmol) in DCM (2.70 mL) at −78° C. The mixture was stirred at −78° C. for 2 min. 2-(cyclopropoxy)ethanol (100 mg, 0.979 mmol) in DCM (2.60 mL) was added, and the mixture was stirred at −78° C. for 1 h. NEt₃ (0.409 mL, 2.94 mmol) was added, and the mixture was stirred at 23° C. for 90 min to form the aldehyde in solution, which was used as such for the next step without purification.

### Step 2: N-[5-(1H-benzimidazol-2-yl)-1-methyl-pyrazol-3-yl]-6-[4-[2-(cyclo-propoxy)ethyl]piperazin-1-yl]pyridine-3-carboxamide

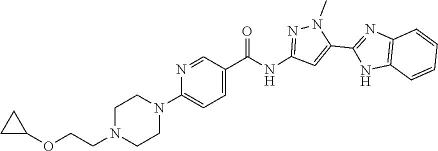 [SMILES: Cn1nc(NC(=O)c2ccc(N3CCN(CCOC4CC4)CC3)nc2)cc1-c1nc2ccccc2[nH]1]

2-(cyclopropoxy)acetaldehyde (in solution from step 1, 98 mg, 0.979 mmol) was added to a solution of N-[5-(1H-benzimidazol-2-yl)-1-methyl-pyrazol-3-yl]-6-piperazin-1-yl-pyridine-3-carboxamide (Compound A-216) (100 mg, 0.248 mmol) in DCM (5.30 mL) at 23° C. Na₂SO₄ was added. NaBH₃CN (31.2 mg, 0.497 mmol) was slowly added at 0° C. The mixture was stirred at 23° C. for 18 h. More 2-(cyclopropoxy)acetaldehyde (in solution from step 1, 98 mg, 0.979 mmol) in DCM (5.30 mL) and Na₂SO₄ were added. The mixture was stirred at 23° C. for 18 h. Water (20 mL) was added. The aq. phase was extracted with EtOAc (2×30 mL), and the combined organic phases were washed with brine (15 mL), dried (Na₂SO₄), filtered, and concentrated. The residue was purified by silica gel chromatography (25 g cartridge) eluting with DCM and MeOH (0-20%) and by preparative HPLC (BEH, C18) eluting with water [10 mM (NH₄)(HCO₃)] and MeCN (37-47%). The necessary fractions were concentrated. The residue was dissolved in DMSO (0.500 mL). NEt₃ (0.500 mL) was added, and the mixture was stirred at 23° C. for 5 min. Water (10 mL) was added, the precipitate was filtered, washed with water (10 mL), and dried to provide the title compound as a solid (25 mg, 21%). ¹H NMR (400 MHz, DMSO-d6) δ 13.05 (s, 1H), 10.83 (s, 1H), 8.79 (d, J=2.4 Hz, 1H), 8.14 (dd, J=9.1, 2.5 Hz, 1H), 7.72 (d, J=7.5 Hz, 1H), 7.53 (d, J=7.7 Hz, 1H), 7.47 (s, 1H), 7.31-7.18 (m, 2H), 6.88 (d, J=9.1 Hz, 1H), 4.30 (s, 3H), 3.64-3.60 (m, 4H), 3.58 (t, J=5.9 Hz, 2H), 3.31-3.27 (m, 1H), 2.55-2.51 (m, 6H), 0.49-0.45 (m, 2H), 0.45-0.39 (m, 2H). m/z (ES⁺), [M+H]⁺ 487.3; HPLC (A05) t_{R}=2.66 min.

### EXAMPLE B81

### Preparation of Compound A-267

### Step 1: 6-[(3aR,6aS)-1,3,3a,4,6,6a-hexahydrofuro[3,4-c]pyrrol-5-yl]-N-[5-(1H-benzimidazol-2-yl)-1-methyl-pyrazol-3-yl]pyridine-3-carboxamide

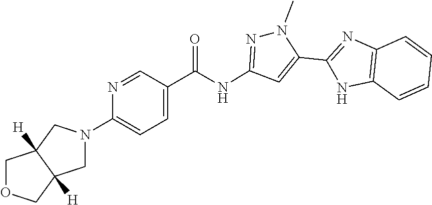 [SMILES: Cn1nc(NC(=O)c2ccc(N3C[C@H]4COC[C@H]4C3)nc2)cc1-c1nc2ccccc2[nH]1]

(3aR,6aS)-3,3a,4,5,6,6a-hexahydro-1H-furo[3,4-c]pyrrole (19.2 mg, 0.170 mmol) was added to a solution of N-[5-(1H-benzimidazol-2-yl)-1-methyl-pyrazol-3-yl]-6-chloro-pyridine-3-carboxamide (60 mg, 0.170 mmol) and DIEA (0.0873 mL, 0.510 mmol) in DMSO (1.50 mL). The mixture was heated to 110° C. and stirred for 18 h. Water (20 mL) was added, and the precipitate was filtered. The residue was purified by silica gel chromatography (12 g cartridge) eluting with DCM and MeOH (0-10%) to provide the title compound as a solid (51 mg, 70%). ¹H NMR (500 MHz, DMSO-d6) δ 13.05 (s, 1H), 10.80 (s, 1H), 8.79 (d, J=2.4 Hz, 1H), 8.14 (dd, J=8.9, 2.5 Hz, 1H), 7.71 (d, J=6.9 Hz, 1H), 7.53 (d, J=7.3 Hz, 1H), 7.47 (s, 1H), 7.30-7.18 (m, 2H), 6.55 (d, J=9.0 Hz, 1H), 4.30 (s, 3H), 3.84 (dd, J=8.8, 6.6 Hz, 2H), 3.68 (dd, J=11.1, 7.7 Hz, 2H), 3.60 (dd, J=8.9, 3.4 Hz, 2H), 3.43 (dd, J=11.2, 3.0 Hz, 2H), 3.09-3.00 (m, 2H). m/z (ES⁺), [M+H]⁺ 430.2. HPLC (A05) t_{R}=2.07 min.

### EXAMPLE B82

### Preparation of Compound A-268

### Step 1: N-[5-(1H-benzimidazol-2-yl)-1-methyl-pyrazol-3-yl]-6-(2-oxa-7-azaspiro[3.4]octan-7-yl)pyridine-3-carboxamide

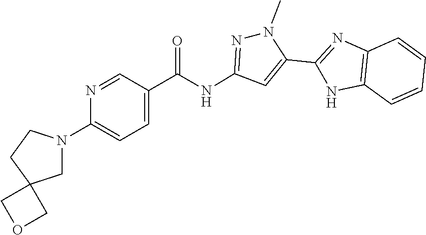 [SMILES: Cn1nc(NC(=O)c2ccc(N3CCC4(COC4)C3)nc2)cc1-c1nc2ccccc2[nH]1]

2-oxa-7-azaspiro[3.4]octane (19.2 mg, 0.17 mmol) was added to a solution of N-[5-(1H-benzimidazol-2-yl)-1-methyl-pyrazol-3-yl]-6-chloro-pyridine-3-carboxamide (60 mg, 0.170 mmol) in DMSO (1.50 mL). The mixture was heated to 110° C. and stirred for 18 h. Water (60 mL) was added, and the aq. phase was extracted with EtOAc (3×70 mL). The combined organic phases were washed with brine (60 mL), dried (Na₂SO₄), filtered, and concentrated. The residue was purified by silica gel chromatography (12 g cartridge), eluting with DCM and MeOH (0-10%) to provide the title compound as a solid (21 mg, 29%). ¹H NMR (400 MHz, DMSO-d6) δ 13.00 (s, 1H), 10.73 (s, 1H), 8.75 (d, J=2.4 Hz, 1H), 8.09 (dd, J=8.9, 2.5 Hz, 1H), 7.75-7.59 (m, 1H), 7.58-7.44 (m, 1H), 7.42 (s, 1H), 7.27-7.11 (m, 2H), 6.47 (d, J=8.9 Hz, 1H), 4.56 (d, J=6.0 Hz, 2H), 4.49 (d, J=6.1 Hz, 2H), 4.25 (s, 3H), 3.68 (s, 2H), 3.43 (t, J=6.9 Hz, 2H), 2.23 (t, J=7.0 Hz, 2H). m/z (ES⁺), [M+H]⁺ 430.6. HPLC (A05) t_{R}=2.05 min.

### EXAMPLE B83

### Preparation of Compound A-269

### Step 1: tert-butyl 4-(2-hydroxyacetyl)piperazine-1-carboxylate

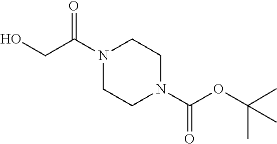 [SMILES: CC(C)(C)OC(=O)N1CCN(C(=O)CO)CC1]

2-Hydroxyacetic acid (200 mg, 2.63 mmol) and HATU (1.00 g, 2.63 mmol) were dissolved in DMF (15 mL) at 0° C. DIEA (0.9 mL, 5.26 mmol) was added, and the mixture was stirred at 0° C. for 5 min. Tert-butyl piperazine-1-carboxylate (1469 mg, 7.89 mmol) was added. The mixture was stirred at 23° C. for 72 h. The mixture was concentrated, and water (100 mL) was added. The aq. phase was extracted with EtOAc (3×50 mL), and the combined organic phases were washed with 0.1M aq. citric acid (60 mL), brine (50 mL), dried (Na₂SO₄), filtered and concentrated to provide the title compound as a solid (572 mg, 89%). ¹H NMR (400 MHz, DMSO-d6) δ 4.60 (t, J=5.2 Hz, 1H), 4.08 (d, J=1.7 Hz, 2H), 3.46-3.40 (m, 2H), 3.30-3.25 (m, 4H), 2.77-2.70 (m, 2H), 1.40 (s, 9H).

### Step 2: 2-hydroxy-1-piperazin-1-yl-ethanone hydrochloride

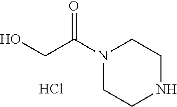

4.0 M HCl in 1,4-Dioxane (1.76 mL, 7.02 mmol) was added to a solution of tert-butyl 4-(2-hydroxyacetyl)piperazine-1-carboxylate (572 mg, 2.34 mmol) in 1,4-Dioxane (5 mL). The mixture was stirred at 23° C. for 18 h. The precipitate was filtered and dried to provide the title compound as a solid (352 mg, 83%). ¹H NMR (400 MHz, DMSO-d6) δ 9.28-9.06 (m, 2H), 4.12 (s, 2H), 3.68-3.63 (m, 2H), 3.62-3.57 (m, 2H), 3.12-3.03 (m, 4H).

### Step 3: N-[5-(1H-benzimidazol-2-yl)-1-methyl-pyrazol-3-yl]-6-[4-(2-hydroxyacetyl)piperazin-1-yl]pyridine-3-carboxamide

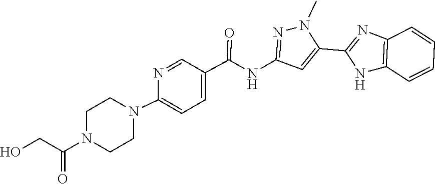 [SMILES: Cn1nc(NC(=O)c2ccc(N3CCN(C(=O)CO)CC3)nc2)cc1-c1nc2ccccc2[nH]1]

2-hydroxy-1-piperazin-1-yl-ethanone hydrochloride (30.7 mg, 0.170 mmol) was added to a solution of N-[5-(1H-benzimidazol-2-yl)-1-methyl-pyrazol-3-yl]-6-chloro-pyridine-3-carboxamide (60 mg, 0.170 mmol) in DMSO (1.50 mL). The mixture was heated to 110° C. and stirred for 48 h. Water (20 mL) was added, and the precipitate was filtered. The residue was purified by silica gel chromatography (12 g cartridge) eluting with DCM and MeOH (0-15%) and by preparative HPLC (BEH C18 30 ×150) eluting with water (10 mM (NH₄)(HCO₃)) and MeCN (24-34%) to provide the title compound as a solid (4.2 mg, 5%). ¹H NMR (400 MHz, DMSO-d6) δ 13.05 (s, 1H), 10.85 (s, 1H), 8.82 (d, J=2.5 Hz, 1H), 8.18 (dd, J=9.0, 2.5 Hz, 1H), 7.72-7.51 (m, 2H), 7.46 (s, 1H), 7.27-7.17 (m, 2H), 6.92 (d, J=9.1 Hz, 1H), 4.65 (t, J=5.4 Hz, 1H), 4.30 (s, 3H), 4.15 (d, J=5.2 Hz, 2H), 3.74-3.63 (m, 4H), 3.62-3.53 (m, 2H), 3.53-3.42 (m, 2H). m/z (ES⁺), [M+H]⁺ 461.8. HPLC (A05) t_{R}=1.95 min.

### EXAMPLE B84

### Preparation of Compound A-270

### Step 1: N-[5-(1H-benzimidazol-2-yl)-1-methyl-pyrazol-3-yl]-6-[3-(hydroxymethyl)azetidin-1-yl]pyridine-3-carboxamide

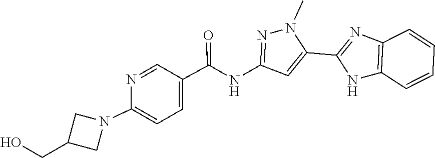 [SMILES: Cn1nc(NC(=O)c2ccc(N3CC(CO)C3)nc2)cc1-c1nc2ccccc2[nH]1]

Azetidin-3-ylmethanol (14.8 mg, 0.170 mmol) was added to a solution of N-[5-(1H-benzimidazol-2-yl)-1-methyl-pyrazol-3-yl]-6-chloro-pyridine-3-carboxamide (60 mg, 0.17 mmol) and DIEA (0.0873 mL, 0.510 mmol) in DMSO (1.5 mL). The mixture was heated to 110° C. and stirred for 18 h. Water (60 mL) was added, and the aq. phase was extracted with EtOAc (3×50 mL). The combined organic phases were washed with brine (50 mL), dried (Na₂SO₄), filtered, and concentrated. The residue was purified by silica gel chromatography (12 g cartridge) eluting with DCM and MeOH (0-20%) to provide the title compound as a solid (45 mg, 66%). ¹H NMR (400 MHz, DMSO-d6) δ 13.05 (s, 1H), 10.79 (s, 1H), 8.76 (d, J=2.4 Hz, 1H), 8.11 (dd, J=8.8, 2.4 Hz, 1H), 7.77-7.64 (m, 1H), 7.59-7.48 (m, 1H), 7.46 (s, 1H), 7.32-7.14 (m, 2H), 6.38 (d, J=8.8 Hz, 1H), 4.81 (t, J=5.3 Hz, 1H), 4.30 (s, 3H), 4.04 (t, J=8.3 Hz, 2H), 3.78 (dd, J=8.6, 5.4 Hz, 2H), 3.59 (t, J=5.7 Hz, 2H), 2.89-2.76 (m, 1H). m/z (ES⁺), [M+H]⁺ 404.2. HPLC (A05) t_{R}=1.93 min.

### EXAMPLE B85

### Preparation of Compound A-271

### Step 1: tert-butyl 4-(2-methoxyacetyl)piperazine-1-carboxylate

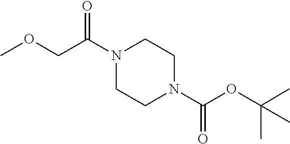 [SMILES: COCC(=O)N1CCN(C(=O)OC(C)(C)C)CC1]

2-methoxyacetic acid (237 mg, 2.63 mmol) and HATU (1.00 g, 2.63 mmol) were dissolved in DMF (15 mL) at 0° C. DIEA (0.900 mL, 5.26 mmol) was added, and the mixture was stirred at 0° C. for 5 min. Tert-butyl piperazine-1-carboxylate (490 mg, 2.63 mmol) was added. The mixture was stirred at 23° C. for 72 h. The mixture was concentrated, and water (100 mL) was added. The aq. phase was extracted with EtOAc (3×50 mL), and the combined organic phases were washed with 0.1 M aq. citric acid (60 mL), brine (50 mL), dried (Na₂SO₄), filtered, and concentrated to provide the title compound as a solid (426 mg, 63%). ¹H NMR (400 MHz, DMSO-d6) δ 4.08 (s, 2H), 3.44-3.29 (m, 8H), 3.28 (s, 3H), 1.41 (s, 9H).

### Step 2: 2-methoxy-1-piperazin-1-yl-ethanone hydrochloride

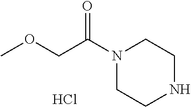

4 M HCl in 1,4-Dioxane (1.24 mL, 4.95 mmol) was added to a solution of tert-butyl 4-(2-methoxyacetyl)piperazine-1-carboxylate (426 mg, 1.65 mmol) in 1,4-Dioxane (3 mL). The mixture was stirred at 23° C. for 18 h. The precipitate was filtered and dried to provide the title compound as a solid (202 mg, 63%). ¹H NMR (400 MHz, DMSO-d6) δ 9.37 (s, 2H), 4.12 (s, 2H), 3.68-3.58 (m, 4H), 3.29 (s, 3H), 3.11-3.00 (m, 4H).

### Step 3: N-[5-(1H-benzimidazol-2-yl)-1-methyl-pyrazol-3-yl]-4-[4-(2-hydroxyacetyl)piperazin-1-yl]benzamide

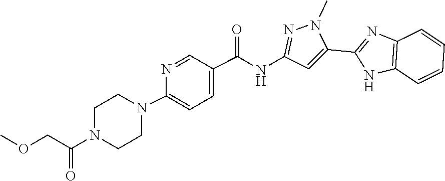 [SMILES: COCC(=O)N1CCN(c2ccc(C(=O)Nc3cc(-c4nc5ccccc5[nH]4)n(C)n3)cn2)CC1]

2-methoxy-1-piperazin-1-yl-ethanone hydrochloride (33.1 mg, 0.17 mmol) was added to a solution of N-[5-(1H-benzimidazol-2-yl)-1-methyl-pyrazol-3-yl]-6-chloro-pyridine-3-carboxamide (60 mg, 0.17 mmol) in DMSO (1.5 mL). The mixture was heated to 110° C. and stirred for 18 h. Water (60 mL) was added, and the aq. phase was extracted with EtOAc (3×50 mL). The combined organic phases were washed with brine (50 mL), dried (Na₂SO₄), filtered, and concentrated. The residue was purified by silica gel chromatography (12 g cartridge) eluting with DCM and MeOH (0-10%) to provide the title compound as a solid (18.5 mg, 23%). ¹H NMR (400 MHz, DMSO-d6) δ 13.06 (s, 1H), 10.86 (s, 1H), 8.82 (d, J=2.4 Hz, 1H), 8.18 (dd, J=9.0, 2.5 Hz, 1H), 7.78-7.64 (m, 1H), 7.63-7.50 (m, 1H), 7.47 (s, 1H), 7.32-7.16 (m, 2H), 6.92 (d, J=9.1 Hz, 1H), 4.30 (s, 3H), 4.14 (s, 2H), 3.72-3.64 (m, 4H), 3.59-3.49 (m, 4H), 3.31 (s, 3H). m/z (ES⁺), [M+H]⁺ 475.8. HPLC (A05) t_{R}=2.01 min.

### EXAMPLE B86

### Preparation of Compound A-272

### Step 1: N-[5-(1H-benzimidazol-2-yl)-1-methyl-pyrazol-3-yl]-6-[3-(methoxymethyl)azetidin-1-yl]pyridine-3-carboxamide

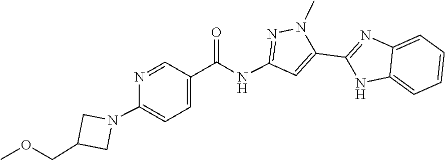 [SMILES: COCC1CN(c2ccc(C(=O)Nc3cc(-c4nc5ccccc5[nH]4)n(C)n3)cn2)C1]

N-[5-(1H-benzimidazol-2-yl)-1-methyl-pyrazol-3-yl]-6-chloro-pyridine-3-carboxamide (60 mg, 0.170 mmol), 3-(methoxymethyl)azetidine hydrochloride (23.4 mg, 0.170 mmol) and DIEA (0.0873 mL, 0.510 mmol) were dissolved in DMSO (1.50 mL). The mixture was stirred at 110° C. for 24 h. After cooling at 23° C., water (5 mL) was added. The aq. phase was extracted with EtOAc (2×10 mL), and the combined organic phases were washed with brine (5 mL), dried (Na₂SO₄), filtered, and concentrated. The residue was purified by silica gel chromatography (12 g cartridge) eluting with DCM and MeOH (0-15%) and by preparative HPLC (BEH C18 30×150) eluting with water (10 mM (NH₄)(HCO₂)) and MeCN (30-40%) to provide the title compound as a solid (54 mg, 76%). ¹H NMR (400 MHz, DMSO-d6) δ 10.81 (s, 1H), 8.76 (d, J=2.4 Hz, 1H), 8.12 (dd, J=8.8, 2.4 Hz, 1H), 7.76-7.50 (m, 2H), 7.46 (s, 1H), 7.28-7.20 (m, 2H), 6.39 (d, J=8.8 Hz, 1H), 4.30 (s, 3H), 4.09 (t, J=8.4 Hz, 2H), 3.76 (dd, J=8.6, 5.5 Hz, 2H), 3.54 (d, J=6.4 Hz, 2H), 3.30 (s, 3H), 3.02-2.92 (m, 1H). m/z (ES⁺), [M+H]⁺ 418.3. HPLC (A05) t_{R}=2.98 min.

### EXAMPLE B87

### Preparation of Compound A-273

### Step 1: N-[5-(1H-benzimidazol-2-yl)-1-methyl-pyrazol-3-yl]-6-(1-oxa-8-azaspiro[4.5]decan-8-yl)pyridine-3-carboxamide

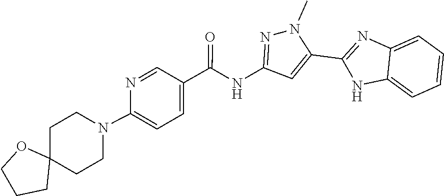 [SMILES: Cn1nc(NC(=O)c2ccc(N3CCC4(CCCO4)CC3)nc2)cc1-c1nc2ccccc2[nH]1]

N-[5-(1H-benzimidazol-2-yl)-1-methyl-pyrazol-3-yl]-6-chloro-pyridine-3-carboxamide (60 mg, 0.170 mmol), 1-oxa-8-azaspiro[4.5]decane hydrochloride (30.2 mg, 0.170 mmol) and DIEA (0.0873 mL, 0.510 mmol) were dissolved in DMSO (1.50 mL). The mixture was stirred at 110° C. for 24 h. After cooling at 23° C., water (5 mL) was added. The aq. phase was extracted with EtOAc (2×10 mL), and the combined organic phases were washed with brine (5 mL), dried (Na₂SO₄), filtered, and concentrated. The residue was purified by silica gel chromatography (12 g cartridge) eluting with DCM and MeOH (0-10%) and by preparative HPLC (BEH C18 30×150) eluting with water (10 mM (NH₄)(HCO₂)) and MeCN (27-37%) to provide the title compound as a solid (40 mg, 51%). ¹H NMR (400 MHz, DMSO-d6) δ 10.83-10.77 (m, 1H), 8.78 (d, J=2.5 Hz, 1H), 8.13 (dd, J=9.1, 2.5 Hz, 1H), 7.78-7.65 (m, 1H), 7.62-7.49 (m, 1H), 7.47 (s, 1H), 7.30-7.18 (m, 2H), 6.90 (d, J=9.1 Hz, 1H), 4.30 (s, 3H), 3.86 (dt, J=13.0, 4.8 Hz, 2H), 3.77 (t, J=6.7 Hz, 2H), 3.60-3.50 (m, 2H), 1.98-1.84 (m, 2H), 1.70 (dd, J=8.4, 6.5 Hz, 2H), 1.62-1.53 (m, 4H). m/z (ES⁺), [M+H]⁺ 458.3. HPLC (A05) t_{R}=3.64 min.

### EXAMPLE B88

### Preparation of Compound A-274

### Step 1: N-[5-(1H-benzimidazol-2-yl)-1-methyl-pyrazol-3-yl]-6-(6-hydroxy-2-azaspiro[3.3]heptan-2-yl)pyridine-3-carboxamide

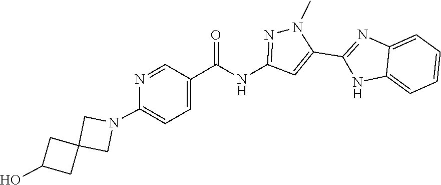 [SMILES: Cn1nc(NC(=O)c2ccc(N3CC4(CC(O)C4)C3)nc2)cc1-c1nc2ccccc2[nH]1]

N-[5-(1H-benzimidazol-2-yl)-1-methyl-pyrazol-3-yl]-6-chloro-pyridine-3-carboxamide (60 mg, 0.170 mmol), 2-azaspiro[3.3]heptan-6-ol hydrochloride (25.4 mg, 0.170 mmol) and DIEA (0.0873 mL, 0.510 mmol) were dissolved in DMSO (1.5 mL). The mixture was stirred at 100° C. for 72 h. After cooling at 23° C., water (5 mL) was added. The aq. phase was extracted with EtOAc (2×10 mL), and the combined organic phases were washed with brine (5 mL), dried (Na₂SO₄), filtered, and concentrated. The residue was purified by silica gel chromatography (12 g cartridge) eluting with DCM and MeOH (0-15%) and by preparative HPLC (BEH C18 30×150) eluting with water (10 mM (NH₄)(HCO₂)) and MeCN (27-37%) to provide the title compound as a solid (15 mg, 21%). ¹H NMR (400 MHz, DMSO-d6) δ 13.06 (s, 1H), 10.70 (s, 1H), 8.74 (d, J=2.3 Hz, 1H), 8.11 (dd, J=8.8, 2.4 Hz, 1H), 7.60-7.49 (m, 2H), 7.37-7.29 (m, 1H), 7.16-7.05 (m, 2H), 6.36 (d, J=8.8 Hz, 1H), 5.06 (d, J=6.0 Hz, 1H), 4.30 (s, 3H), 4.03-4.01 (m, 1H), 4.01 (s, 2H), 3.96 (s, 2H), 2.48-2.44 (m, 2H), 2.08-1.98 (m, 2H). m/z (ES⁺), [M+H]⁺ 430.3. HPLC (A05) t_{R}=2.67 min.

### EXAMPLE B89

### Preparation of Compound A-275

### Step 1: N-[5-(1H-benzimidazol-2-yl)-1-methyl-pyrazol-3-yl]-6-(2-oxa-6-azaspiro[3.3]heptan-6-yl)pyridine-3-carboxamide

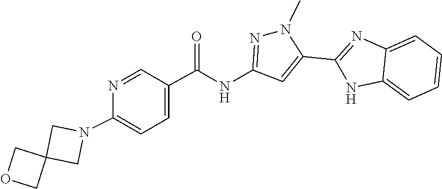 [SMILES: Cn1nc(NC(=O)c2ccc(N3CC4(COC4)C3)nc2)cc1-c1nc2ccccc2[nH]1]

N-[5-(1H-benzimidazol-2-yl)-1-methyl-pyrazol-3-yl]-6-chloro-pyridine-3-carboxamide (60 mg, 0.170 mmol), 2-oxa-6-azaspiro[3.3]heptane (16.9 mg, 0.170 mmol) and DIEA (0.0873 mL, 0.510 mmol) were dissolved in DMSO (1.50 mL). The mixture was stirred at 110° C. for 18 h. After cooling to 23° C., water (5 mL) was added. The mixture was filtered, and the solid was purified by silica gel chromatography (12 g cartridge) eluting with DCM and MeOH (0-10%) to provide the title compound as a solid (21 mg, 30%). ¹H NMR (400 MHz, DMSO-d6) δ 10.83 (s, 1H), 8.77 (d, J=2.3 Hz, 1H), 8.13 (dd, J=8.7, 2.3 Hz, 1H), 7.76-7.50 (m, 2H), 7.46 (s, 1H), 7.29-7.20 (m, 2H), 6.42 (d, J=8.8 Hz, 1H), 4.74 (s, 4H), 4.30 (s, 3H), 4.21 (s, 4H). m/z (ES⁺), [M+H]⁺ 416.3. HPLC (A05) t_{R}=2.69 min.

### EXAMPLE B90

### Preparation of Compound A-276

### Step 1: benzyl 4-[5-[[5-(1H-benzimidazol-2-yl)-1-methyl-pyrazol-3-yl]carbamoyl]-2-pyridyl]-2-(hydroxymethyl)piperazine-1-carboxylate

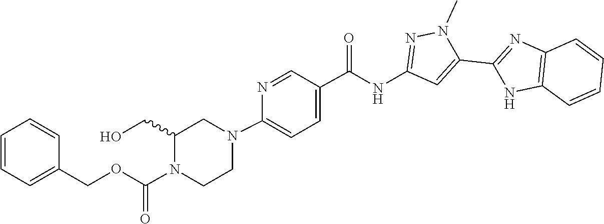 [SMILES: Cn1nc(NC(=O)c2ccc(N3CCN(C(=O)OCc4ccccc4)C(CO)C3)nc2)cc1-c1nc2ccccc2[nH]1]

Benzyl 2-(hydroxymethyl)piperazine-1-carboxylate (42.6 mg, 0.170 mmol) was added to a solution of N-[5-(1H-benzimidazol-2-yl)-1-methyl-pyrazol-3-yl]-6-chloro-pyridine-3-carboxamide (60 mg, 0.170 mmol) and DIEA (0.0873 mL, 0.510 mmol) in DMSO (1.50 mL). The mixture was heated to 110° C. and stirred for 18 h. Water (20 mL) was added, and the precipitate was filtered. The residue was purified by silica gel chromatography (12 g cartridge) eluting with DCM and MeOH (0-10%) to provide the title compound as a solid (51.4 mg, 53%). ¹H NMR (400 MHz, DMSO-d6) δ 13.05 (s, 1H), 10.84 (s, 1H), 8.80 (d, J=2.3 Hz, 1H), 8.17 (dd, J=9.0, 2.2 Hz, 1H), 7.75-7.65 (m, 1H), 7.58-7.49 (m, 1H), 7.47 (s, 1H), 7.43-7.29 (m, 5H), 7.29-7.18 (m, 2H), 6.85 (d, J=9.1 Hz, 1H), 5.16-5.08 (m, 2H), 4.91 (t, J=5.3 Hz, 1H), 4.37 (d, J=13.7 Hz, 1H), 4.33-4.22 (m, 4H), 4.19-4.11 (m, 1H), 3.95-3.89 (m, 1H), 3.48-3.40 (m, 2H), 3.30-3.17 (m, 2H), 3.07 (td, J=12.3, 3.5 Hz, 1H). m/z (ES⁺), [M+H]⁺ 567.2. HPLC (A05) t_{R}=2.29 min.

### Step 2: N-[5-(1H-benzimidazol-2-yl)-1-methyl-pyrazol-3-yl]-6-(3-oxo-5,6,8,8a-tetrahydro-1H-oxazolo[3,4-a]pyrazin-7-yl)pyridine-3-carboxamide

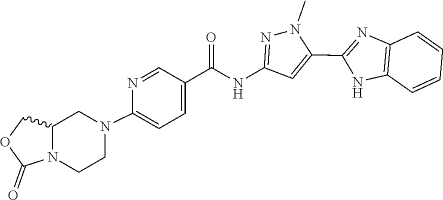 [SMILES: Cn1nc(NC(=O)c2ccc(N3CCN4C(=O)OCC4C3)nc2)cc1-c1nc2ccccc2[nH]1]

A solution of benzyl 4-[5-[[5-(1H-benzimidazol-2-yl)-1-methyl-pyrazol-3-yl]carbamoyl]-2-pyridyl]-2-(hydroxymethyl)piperazine-1-carboxylate (51 mg, 0.0900 mmol) in DMF (0.800 mL) was added dropwise to a solution of 60 wt. % NaH in mineral oil (10.3 mg, 0.270 mmol) in DMF (1 mL) at 0° C. The mixture was warmed to 23° C. and stirred for 2 h. Water (50 mL) was added, and the aq. phase was extracted with EtOAc (3×40 mL). The combined organic phases were washed with brine (30 mL), dried (Na₂SO₄), filtered, and concentrated. The residue was purified by silica gel chromatography (12 g cartridge) eluting with DCM and MeOH (0-10%) to provide the title compound as a solid (19.6 mg, 48%). ¹H NMR (400 MHz, DMSO-d6) δ 13.06 (s, 1H), 10.88 (s, 1H), 8.82 (d, J=3.1 Hz, 1H), 8.20 (dd, J=9.1, 2.5 Hz, 1H), 7.70 (s, 1H), 7.55 (s, 1H), 7.47 (s, 1H), 7.25 (d, J=6.6 Hz, 2H), 6.97 (d, J=9.1 Hz, 1H), 4.71-4.63 (m, 1H), 4.49 (d, J=12.9 Hz, 1H), 4.43 (t, J=8.6 Hz, 1H), 4.30 (s, 3H), 4.03 (dd, J=8.9, 5.5 Hz, 1H), 3.92-3.82 (m, 1H), 3.66 (dd, J=13.5, 3.2 Hz, 1H), 3.10-2.92 (m, 2H), 2.88 (dd, J=13.1, 11.1 Hz, 1H). m/z (ES⁺), [M+H]⁺ 459.4. HPLC (A05) t_{R}=2.04 min.

### EXAMPLE B91

### Preparation of Compound A-277

### Step 1: 2-(3-amino-4-nitro-phenoxy)ethanol

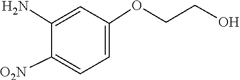 [SMILES: Nc1cc(OCCO)ccc1[N+](=O)[O-]]

K₂CO₃ (797 mg, 0.00576 mol) was added to a solution of 5-fluoro-2-nitro-aniline (600 mg, 0.00384 mol) in ethylene glycol (20 mL). The mixture was stirred at 110° C. for 18 h. Water (100 mL) was added, and the precipitate was filtered to provide the title compound as a solid (724 mg, 95%). ¹H NMR (400 MHz, DMSO-d6) δ 7.91 (d, J=9.6 Hz, 1H), 7.46 (s, 2H), 6.44 (d, J=2.7 Hz, 1H), 6.25 (dd, J=9.5, 2.7 Hz, 1H), 4.91 (t, J=5.4 Hz, 1H), 3.99 (t, J=4.9 Hz, 2H), 3.71 (dd, J=9.8, 4.9 Hz, 2H). HPLC (A05) t_{R}=1.81 min.

### Step 2: 2-(3-amino-4-nitro-phenoxy)ethyl tert-butyl carbonate

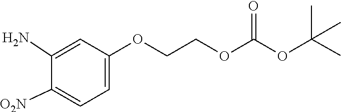 [SMILES: CC(C)(C)OC(=O)OCCOc1ccc([N+](=O)[O-])c(N)c1]

A solution of Boc₂O (793 mg, 3.63 mmol) in dioxane (5 mL) was added dropwise to a solution of 2-(3-amino-4-nitro-phenoxy)ethanol (720 mg, 3.63 mmol), DMAP (44.4 mg, 0.363 mmol) and NEt₃ (0.760 mL, 5.45 mmol) in 1,4-Dioxane (10 mL) at 23° C. The mixture was stirred for 18 h. The mixture was concentrated, and the residue was purified by silica gel chromatography (25 g cartridge) eluting with hexanes and EtOAc (0-50%) to provide the title compound as a solid (661 mg, 61%). ¹H NMR (400 MHz, DMSO-d6) δ 7.92 (d, J=9.5 Hz, 1H), 7.46 (s, 2H), 6.44 (d, J=2.7 Hz, 1H), 6.26 (dd, J=9.5, 2.7 Hz, 1H), 4.33 (dd, J=5.3, 3.5 Hz, 2H), 4.18 (dd, J=5.3, 3.5 Hz, 2H), 1.42 (s, 9H). HPLC (A05) t_{R}=2.37 min.

### Step 3: 2-[3-(tert-butoxycarbonylamino)-4-nitro-phenoxy]ethyl tert-butyl carbonate

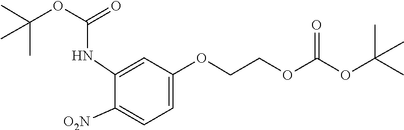 [SMILES: CC(C)(C)OC(=O)Nc1cc(OCCOC(=O)OC(C)(C)C)ccc1[N+](=O)[O-]]

Boc₂O (484 mg, 2.22 mmol) was added to a solution of 2-(3-amino-4-nitro-phenoxy)ethyl tert-butyl carbonate (661 mg, 2.22 mmol), DMAP (27.1 mg, 0.222 mmol) and NEt₃ (0.463 mL, 3.32 mmol) in 1,4-Dioxane (15 mL). The mixture was stirred at 23° C. for 18 h. The mixture was concentrated, and the residue was purified by silica gel chromatography (25 g cartridge) eluting with hexanes and EtOAc (0-30%) to provide the title compound as a solid (285 mg, 32%). ¹H NMR (400 MHz, DMSO-d6) δ 9.72 (s, 1H), 8.08 (d, J=9.3 Hz, 1H), 7.57 (d, J=2.7 Hz, 1H), 6.83 (dd, J=9.3, 2.8 Hz, 1H), 4.39-4.33 (m, 2H), 4.30 (dd, J=5.7, 2.8 Hz, 2H), 1.47 (s, 9H), 1.42 (s, 9H). HPLC (A05) t_{R}=2.85 min.

### Step 4: 2-[4-amino-3-(tert-butoxycarbonylamino)phenoxy]ethyl tert-butyl carbonate

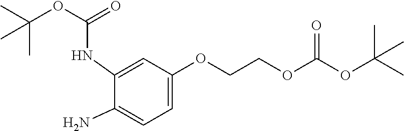 [SMILES: CC(C)(C)OC(=O)Nc1cc(OCCOC(=O)OC(C)(C)C)ccc1N]

2-[3-(tert-butoxycarbonylamino)-4-nitro-phenoxy]ethyl tert-butyl carbonate (280 mg, 0.703 mmol) was solubilized in MeOH (30 mL) and transferred in a flask purged with nitrogen containing Pd/C (10.0%, 74.8 mg, 0.0703 mmol). The mixture was stirred at 23° C. for 18 h under an atmosphere of hydrogen. The flask was purged with nitrogen, the solution was filtered on celite, and the filtrate was concentrated to provide the title compound as a solid (258 mg, 99%). ¹H NMR (400 MHz, DMSO-d6) δ 8.27 (s, 1H), 6.96 (s, 1H), 6.62 (d, J=8.7 Hz, 1H), 6.48 (dd, J=8.6, 2.9 Hz, 1H), 4.48 (s, 2H), 4.29-4.21 (m, 2H), 4.02-3.96 (m, 2H), 1.46 (s, 9H), 1.42 (s, 9H). m/z (ES⁺), [M+H]⁺ 369.2. HPLC (A05) t_{R}=2.59 min.

### Step 5: 2-[3-(tert-butoxycarbonylamino)-4-[[5-[(3-chloro-4-methoxy-benzoyl)amino]-2-[(4-methoxyphenyl)methyl]pyrazole-3-carbonyl]amino]phenoxy]ethyl tert-butyl carbonate

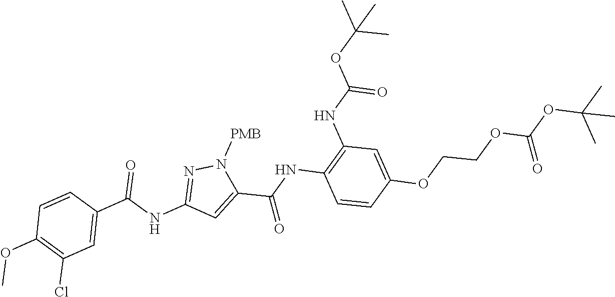

5-[(3-chloro-4-methoxy-benzoyl)amino]-2-[(4-methoxyphenyl)methyl]pyrazole-3-carboxylic acid (113 mg, 0.271 mmol) and HATU (0.103 g, 0.271 mmol) were dissolved in DMF (2.50 mL) at 0° C. DIEA (0.0929 mL, 0.543 mmol) was added, and the mixture was stirred at 0° C. for 5 min. 2-[4-amino-3-(tert-butoxycarbonylamino)phenoxy]ethyl tert-butyl carbonate (100 mg, 0.271 mmol) was added. The mixture was warmed to 23° C., and stirred for a total of 18 h. The mixture was concentrated, and the residue was purified by silica gel chromatography (12 g cartridge) eluting with DCM and MeOH (0-10%) to provide the title compound as a solid (194 mg, 93%). ¹H NMR (400 MHz, DMSO-d6) δ 11.05 (s, 1H), 9.76 (s, 1H), 8.66 (s, 1H), 8.14 (d, J=2.2 Hz, 1H), 8.05 (dd, J=8.7, 2.2 Hz, 1H), 7.52 (s, 1H), 7.35-7.30 (m, 1H), 7.28-7.18 (m, 4H), 6.90-6.83 (m, 2H), 6.74 (dd, J=8.8, 2.8 Hz, 1H), 5.61 (s, 2H), 4.36-4.29 (m, 2H), 4.19-4.13 (m, 2H), 3.94 (s, 3H), 3.71 (s, 3H), 1.45 (s, 9H), 1.44 (s, 9H). m/z (ES⁺), [M+H]⁺ 767.8. HPLC (A05) t_{R}=2.86 min.

### Step 6: 2-[[2-[5-[(3-chloro-4-methoxy-benzoyl)amino]-2-[(4-methoxyphenyl)-methyl]pyrazol-3-yl]-1H-benzimidazol-5-yl]oxy]ethyl acetate

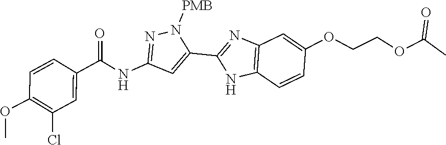

AcOH (5 mL) was added to 2-[3-(tert-butoxycarbonylamino)-4-[[5-[(3-chloro-4-methoxy-benzoyl)amino]-2-[(4-methoxyphenyl)methyl]pyrazole-3-carbonyl]amino]phenoxy]ethyl tert-butyl carbonate (194 mg, 0.253 mmol), the mixture was heated to 110° C. and stirred for 18 h. The mixture was concentrated, and EtOAc (100 mL) was added. The organic phase was washed with sat. aq. NaHCO₃ (50 mL), water (50 mL), brine (50 mL), dried (Na₂SO₄), filtered, and concentrated. The residue was purified by silica gel chromatography (12 g cartridge) eluting with DCM and MeOH (0-10%) to provide the title compound as a solid (79.1 mg, 53%) in a mixture of tautomers. ¹H NMR (400 MHz, DMSO-d6) δ [13.02] 12.99 (s, 1H), 11.06 (s, 1H), 8.15 (d, J=2.2 Hz, 1H), 8.07 (dd, J=8.7, 2.2 Hz, 1H), 7.60 [7.42] (d, J=8.7 Hz, 1H), 7.50 (s, 1H), 7.30-7.26 [7.01] (m, 2H), 7.23 (dd, J=9.3, 2.6 Hz, 2H), 6.95-6.88 (m, 1H), 6.85 (dd, J=8.8, 1.4 Hz, 2H), 6.02 [6.00] (s, 2H), 4.40-4.32 (m, 2H), 4.27-4.20 [4.20-4.15] (m, 2H), 3.94 (s, 3H), 3.69 [3.69] (s, 3H), 2.07 [2.06] (s, 3H). m/z (ES⁺), [M+H]⁺ 591.2. HPLC (A05) t_{R}=2.65 min.

### Step 7: 3-chloro-N-[5-[5-(2-hydroxyethoxy)-1H-benzimidazol-2-yl]-1H-pyrazol-3-yl]-4-methoxy-benzamide

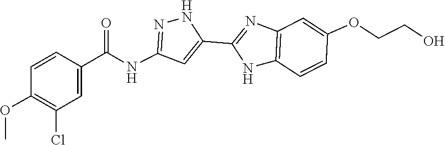 [SMILES: COc1ccc(C(=O)Nc2cc(-c3nc4cc(OCCO)ccc4[nH]3)[nH]n2)cc1Cl]

NaOH (0.0107 g, 0.268 mmol) was added to a solution of 2-[[2-[5-[(3-chloro-4-methoxy-benzoyl)amino]-2-[(4-methoxyphenyl)methyl]pyrazol-3-yl]-1H-benzimidazol-5-yl]oxy]ethyl acetate (79 mg, 0.134 mmol) in MeOH (1 mL) and water (1 mL). The mixture was stirred at 23° C. for 2 h. The mixture was concentrated, water (10 mL) was added, and the precipitate was filtered. TFA (1 mL) was added to the solid, and the mixture was stirred at 23° C. for 2 h. The mixture was concentrated, and the residue was dissolved in DMSO (0.800 mL). NEt₃ (0.800 mL) was added, and the solution was stirred for 18 h. Water (10 mL) was added. The precipitate was filtered and washed with acetone (5 mL), DCM (5 mL) and acetone (5 mL). The solid was purified by preparative HPLC (BEH C18 30×100) eluting with water (10 mM (NH₄)(HCO₃)) and MeCN (26-36%) to provide the title compound as a solid (12.1 mg, 21%) in 4:1 mixture of tautomers. ¹H NMR (400 MHz, DMSO-d6) δ 13.40 (s, 1H), 12.86 [12.56] (s, 1H), [11.34] 10.93 (s, 1H), 8.10 (d, J=2.2 Hz, 1H), 8.02 (dd, J=8.7, 2.2 Hz, 1H), 7.46 [7.11] (s, 1H), 7.40-7.28 (m, 1H), 7.24 (d, J=8.2 Hz, 1H), 7.02-6.89 (m, 1H), 6.87-6.73 (m, 1H), 4.82 (s, 1H), 3.98 (t, J=5.0 Hz, 2H), 3.90 (s, 3H), [3.73-3.67] (m, 2H). m/z (ES⁺), [M+H]⁺ 428.9. HPLC (A05) t_{R}=2.03 min.

### EXAMPLE B92

### Preparation of Compound A-278

### Step 1: N-[5-(1H-benzimidazol-2-yl)-1-methyl-pyrazol-3-yl]-6-[(3R,5S)-4-(2-hydroxyethyl)-3,5-dimethyl-piperazin-1-yl]pyridine-3-carboxamide

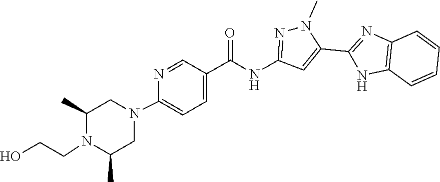 [SMILES: C[C@@H]1CN(c2ccc(C(=O)Nc3cc(-c4nc5ccccc5[nH]4)n(C)n3)cn2)C[C@H](C)N1CCO]

2-[(2R,6S)-2,6-dimethylpiperazin-1-yl]ethanol (17.9 mg, 0.113 mmol) was added to a solution of N-[5-(1H-benzimidazol-2-yl)-1-methyl-pyrazol-3-yl]-6-chloro-pyridine-3-carboxamide (40 mg, 0.113 mmol) and DIEA (0.0582 mL, 0.340 mmol) in DMSO (1 mL). The mixture was heated to 110° C. and stirred for 18 h. EtOAc (100 mL) was added, and the organic phase was washed with water (3×30 mL), brine (40 mL), dried (Na₂SO₄), filtered, and concentrated to provide the title compound as a solid (41.4 mg, 77%). ¹H NMR (400 MHz, DMSO-d6) δ 13.05 (s, 1H), 10.81 (s, 1H), 8.77 (d, J=2.1 Hz, 1H), 8.13 (dd, J=9.1, 2.5 Hz, 1H), 7.72 (d, J=7.2 Hz, 1H), 7.55-7.51 (m, 1H), 7.47 (s, 1H), 7.30-7.20 (m, 2H), 6.90 (d, J=8.8 Hz, 1H), 4.41 (t, J=5.3 Hz, 1H), 4.30 (s, 3H), 4.25 (d, J=9.2 Hz, 2H), 3.45-3.38 (m, 2H), 2.67 (t, J=7.4 Hz, 2H), 2.62-2.55 (m, 4H), 1.10 (s, 3H), 1.09 (s, 3H). m/z (ES⁺), [M+H]⁺ 475.2. HPLC (A05) t_{R}=1.83 min.

### EXAMPLE B93

### Preparation of Compound A-279

### Step 1: N-[5-(1H-benzimidazol-2-yl)-1-methyl-pyrazol-3-yl]-6-[(3R,5S)-4-(2-hydroxyethyl)-3,5-dimethyl-piperazin-1-yl]pyridine-3-carboxamide

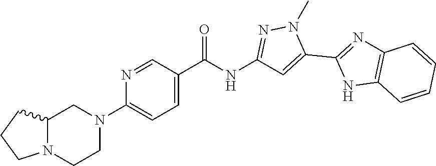 [SMILES: Cn1nc(NC(=O)c2ccc(N3CCN4CCCC4C3)nc2)cc1-c1nc2ccccc2[nH]1]

1,2,3,4,6,7,8,8a-octahydropyrrolo[1,2-a]pyrazine (23.6 mg, 0.187 mmol) was added to a solution of N-[5-(1H-benzimidazol-2-yl)-1-methyl-pyrazol-3-yl]-6-chloro-pyridine-3-carboxamide (60 mg, 0.170 mmol) and DIEA (0.0582 mL, 0.340 mmol) in DMSO (1.50 mL). The mixture was heated to 110° C. and stirred for 42 h. Water (60 mL) was added, and the aq. phase was extracted with EtOAc (3×50 mL). The combined organic phases were washed with brine (50 mL), dried (Na₂SO₄), filtered, and concentrated. The residue was purified by silica gel chromatography (12 g cartridge) eluting with DCM and MeOH (0-10%) to provide the title compound as a solid (49.5 mg, 66%). 1H NMR (400 MHz, DMSO-d6) δ 13.00 (s, 1H), 10.77 (s, 1H), 8.74 (d, J=2.2 Hz, 1H), 8.09 (dd, J=9.0, 2.6 Hz, 1H), 7.66 (d, J=7.4 Hz, 1H), 7.48 (dd, J=6.9, 1.0 Hz, 1H), 7.42 (s, 1H), 7.24-7.12 (m, 2H), 6.86 (d, J=9.2 Hz, 1H), 4.51 (d, J=12.2 Hz, 1H), 4.33 (d, J=11.6 Hz, 1H), 4.25 (s, 3H), 3.05-2.96 (m, 2H), 2.94-2.85 (m, 1H), 2.62-2.52 (m, 1H), 2.11-1.97 (m, 2H), 1.93-1.84 (m, 1H), 1.82-1.75 (m, 1H), 1.72-1.59 (m, 2H), 1.39-1.27 (m, 1H). m/z (ES⁺), [M+H]⁺ 443.2. HPLC (A05) t_{R}=1.93 min.

### EXAMPLE B94

### Preparation of Compound A-280

### Step 1: N-[5-(1H-benzimidazol-2-yl)-1-methyl-pyrazol-3-yl]-6-(3,3-dimethyl-piperazin-1-yl)pyridine-3-carboxamide

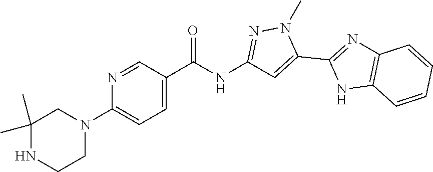 [SMILES: Cn1nc(NC(=O)c2ccc(N3CCNC(C)(C)C3)nc2)cc1-c1nc2ccccc2[nH]1]

2,2-dimethylpiperazine (19.4 mg, 0.170 mmol) was added to a solution of N-[5-(1H-benzimidazol-2-yl)-1-methyl-pyrazol-3-yl]-6-chloro-pyridine-3-carboxamide (60 mg, 0.170 mmol) and DIEA (0.0582 mL, 0.340 mmol) in DMSO (1.50 mL). The mixture was heated to 110° C. and stirred for 42 h. Water (60 mL) was added, and the aq. phase was extracted with EtOAc (3×50 mL). The combined organic phases were washed with brine (50 mL), dried (Na₂SO₄), filtered, and concentrated. The residue was purified by silica gel chromatography (12 g cartridge) eluting with DCM (2.5% NEt₃) and MeOH (0-20%) and by preparative HPLC (BEH C18 30×100) eluting with water (10 mM (NH₄)(HCO₃)) and MeCN (26-36%) to provide the title compound as a solid (19.4 mg, 26%). ¹H NMR (400 MHz, DMSO-d6) δ 13.05 (s, 1H), 10.77 (s, 1H), 8.75 (d, J=2.1 Hz, 1H), 8.11 (dd, J=9.1, 2.5 Hz, 1H), 7.72 (d, J=7.9 Hz, 1H), 7.53 (d, J=7.1 Hz, 1H), 7.47 (s, 1H), 7.31-7.18 (m, 2H), 6.85 (d, J=9.2 Hz, 1H), 4.30 (s, 3H), 3.61-3.53 (m, 2H), 3.41 (s, 2H), 2.85-2.78 (m, 2H), 1.05 (s, 6H). m/z (ES⁺), [M+H]⁺ 431.2. HPLC (A05) t_{R}=1.95 min.

### EXAMPLE B95

### Preparation of Compound A-281

### Step 1: N-[5-(1H-benzimidazol-2-yl)-1-methyl-pyrazol-3-yl]-6-(4,7-diazaspiro[2.5]octan-7-yl)pyridine-3-carboxamide

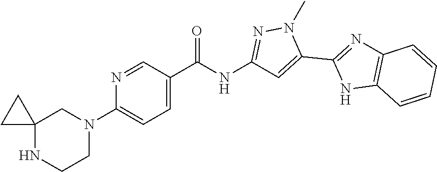 [SMILES: Cn1nc(NC(=O)c2ccc(N3CCNC4(CC4)C3)nc2)cc1-c1nc2ccccc2[nH]1]

4,7-diazaspiro[2.5]octane dihydrochloride (31.5 mg, 0.170 mmol) was added to a solution of N-[5-(1H-benzimidazol-2-yl)-1-methyl-pyrazol-3-yl]-6-chloro-pyridine-3-carboxamide (60 mg, 0.170 mmol) and DIEA (0.116 mL, 0.680 mmol) in DMSO (1.50 mL). The mixture was heated to 110° C. and stirred for 42 h. Water (60 mL) was added, and the aq. phase was extracted with EtOAc (3×50 mL). The combined organic phases were washed with brine (50 mL), dried (Na₂SO₄), filtered, and concentrated. The residue was purified by silica gel chromatography (12 g cartridge) eluting with DCM (2.5% NEt₃) and MeOH (0-20%) and by preparative HPLC (BEH C18 30×100) eluting with water (10 mM (NH₄)(HCO₃)) and MeCN (26-36%) to provide the title compound as a solid (24.3 mg, 33%). ¹H NMR (400 MHz, DMSO-d6) δ 13.05 (s, 1H), 10.79 (s, 1H), 8.76 (d, J=2.2 Hz, 1H), 8.11 (dd, J=9.1, 2.5 Hz, 1H), 7.72 (d, J=7.7 Hz, 1H), 7.53 (d, J=7.2 Hz, 1H), 7.47 (s, 1H), 7.30-7.20 (m, 2H), 6.83 (d, J=9.0 Hz, 1H), 4.30 (s, 3H), 3.60 (dd, J=6.0, 4.1 Hz, 2H), 3.49 (s, 2H), 2.87-2.78 (m, 2H), 0.54-0.48 (m, 2H), 0.48-0.42 (m, 2H). m/z (ES⁺), [M+H]⁺ 429.2. HPLC (A05) t_{R}=1.95 min.

### EXAMPLE B96

### Preparation of Compound A-282

### Step 1: N-[5-(1H-benzimidazol-2-yl)-1-methyl-pyrazol-3-yl]-6-(2-oxa-7-azaspiro[3.5]nonan-7-yl)pyridine-3-carboxamide

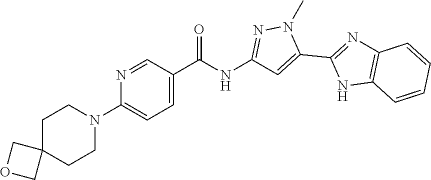 [SMILES: Cn1nc(NC(=O)c2ccc(N3CCC4(CC3)COC4)nc2)cc1-c1nc2ccccc2[nH]1]

2-oxa-7-azaspiro[3.5]nonane oxalic acid (37.6 mg, 0.109 mmol) was added to a solution of N-[5-(1H-benzimidazol-2-yl)-1-methyl-pyrazol-3-yl]-6-chloro-pyridine-3-carboxamide (70 mg, 0.198 mmol) in DMSO (1 mL). The mixture was heated to 110° C. and stirred for 18 h. Water (60 mL) was added, and the aq. phase was extracted with EtOAc (3×50 mL). The combined organic phases were washed with brine (50 mL), dried (Na₂SO₄), filtered, and concentrated. The residue was purified by silica gel chromatography (12 g cartridge) eluting with DCM and MeOH (0-10%) and by preparative HPLC (BEH C18 30×150) eluting with water (10 mM (NH₄)(HCO₂)) and MeCN (33-43%) to provide the title compound as a solid (9.10 mg, 10%). ¹H NMR (400 MHz, DMSO-d6) δ 13.07 (s, 1H), 10.83 (s, 1H), 8.78 (d, J=2.5 Hz, 1H), 8.13 (dd, J=9.1, 2.5 Hz, 1H), 7.78-7.60 (m, 1H), 7.60-7.50 (m, 1H), 7.47 (s, 1H), 7.24 (d, J=4.0 Hz, 2H), 6.92 (d, J=9.1 Hz, 1H), 4.36 (s, 4H), 4.30 (s, 3H), 3.65-3.56 (m, 4H), 1.86-1.76 (m, 4H). m/z (ES⁺), [M+H]⁺ 444.2. HPLC (A05) t_{R}=2.19 min.

### EXAMPLE B97

### Preparation of Compound A-283

### Step 1: N-[5-(1H-benzimidazol-2-yl)-1-methyl-pyrazol-3-yl]-6-[4-hydroxy-4-(2-hydroxyethyl)-1-piperidyl]pyridine-3-carboxamide

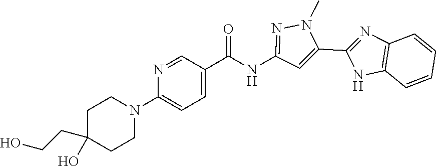 [SMILES: Cn1nc(NC(=O)c2ccc(N3CCC(O)(CCO)CC3)nc2)cc1-c1nc2ccccc2[nH]1]

1-oxa-7-azaspiro[3.5]nonane oxalic acid (47.4 mg, 0.218 mmol) was added to a solution of N-[5-(1H-benzimidazol-2-yl)-1-methyl-pyrazol-3-yl]-6-chloro-pyridine-3-carboxamide (70 mg, 0.198 mmol) in DMSO (1 mL). The mixture was heated to 110° C. and stirred for 96 h. Water (60 mL) was added, and the aq. phase was extracted with EtOAc (3×50 mL). The combined organic phases were washed with brine (50 mL), dried (Na₂SO₄), filtered, and concentrated. The residue was purified by silica gel chromatography (12 g cartridge) eluting with DCM and MeOH (0-10%) and by preparative HPLC (BEH C18 30×100) eluting with water (10 mM (NH₄)(HCO₂)) and MeCN (22-32%) to provide the title compound as a solid (19.7 mg, 21%). ¹H NMR (400 MHz, DMSO-d6) δ 12.64 (s, 1H), 10.74 (s, 1H), 8.70 (d, J=2.4 Hz, 1H), 8.04 (dd, J=9.1, 2.4 Hz, 1H), 7.69-7.55 (m, 1H), 7.55-7.43 (m, 1H), 7.40 (s, 1H), 7.23-7.12 (m, 2H), 6.80 (d, J=9.2 Hz, 1H), 4.35 (s, 2H), 4.23 (s, 3H), 4.00 (d, J=13.1 Hz, 2H), 3.51 (t, J=6.9 Hz, 2H), 3.31-3.21 (m, 2H), 1.54 (t, J=7.0 Hz, 2H), 1.50-1.37 (m, 4H). m/z (ES⁺), [M+H]⁺ 462.3. HPLC (A05) t_{R}=1.95 min.

### EXAMPLE B98

### Preparation of Compound A-284

### Step 1: tert-butyl 3-[5-[[5-(1H-benzimidazol-2-yl)-1-methyl-pyrazol-3-yl]carbamoyl]-2-pyridyl]-3,6-diazabicyclo[3.1.1]heptane-6-carboxylate

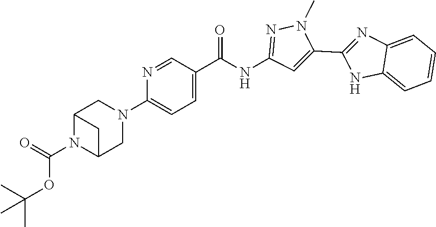 [SMILES: Cn1nc(NC(=O)c2ccc(N3CC4CC(C3)N4C(=O)OC(C)(C)C)nc2)cc1-c1nc2ccccc2[nH]1]

N-[5-(1H-benzimidazol-2-yl)-1-methyl-pyrazol-3-yl]-6-chloro-pyridine-3-carboxamide (70 mg, 0.198 mmol), tert-butyl 3,6-diazabicyclo[3.1.1]heptane-6-carboxylate (39.3 mg, 0.198 mmol) and DIEA (0.102 mL, 0.595 mmol) were dissolved in DMSO (1.75 mL). The mixture was stirred at 85° C. for 18 h. More tert-butyl 3,6-diazabicyclo[3.1.1]heptane-6-carboxylate (19.7 mg, 0.100 mmol) was added, and the mixture was stirred at 85° C. for 24 h. After cooling at 23° C., water (20 mL) was added, and the precipitate was filtered. The residue was purified by silica gel chromatography (12 g cartridge) eluting with DCM and MeOH (0-10%) to provide the title compound as a solid (85 mg, 83%). ¹H NMR (300 MHz, DMSO-d6) δ 13.04 (s, 1H), 10.82 (s, 1H), 8.82 (d, J=2.4 Hz, 1H), 8.17 (dd, J=9.0, 2.4 Hz, 1H), 7.73-7.65 (m, 1H), 7.56-7.47 (m, 1H), 7.46 (s, 1H), 7.28-7.16 (m, 2H), 6.71 (d, J=8.9 Hz, 1H), 4.29 (s, 3H), 4.20 (d, J=6.6 Hz, 2H), 4.11-3.96 (m, 2H), 3.47 (d, J=11.5 Hz, 2H), 1.47 (d, J=8.5 Hz, 1H), 1.29 (s, 1H), 1.26 (s, 9H). m/z (ES⁺), [M+H]⁺ 515.3. HPLC (A05) t_{R}=2.44 min.

### Step 2: N-[5-(1H-benzimidazol-2-yl)-1-methyl-pyrazol-3-yl]-6-(3,6-diazabicyclo[3.1.1]heptan-3-yl)pyridine-3-carboxamide

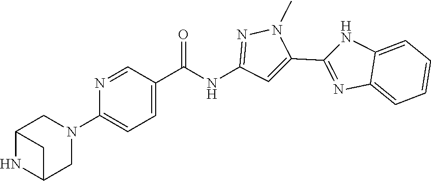 [SMILES: Cn1nc(NC(=O)c2ccc(N3CC4CC(C3)N4)nc2)cc1-c1nc2ccccc2[nH]1]

A solution of tert-butyl 3-[5-[[5-(1H-benzimidazol-2-yl)-1-methyl-pyrazol-3-yl]carbamoyl]-2-pyridyl]-3,6-diazabicyclo[3.1.1]heptane-6-carboxylate (85 mg, 0.165 mmol) in HFIP (3.30 mL) was stirred at 100° C. for 48 h. The mixture was concentrated. The residue was purified by silica gel chromatography (12 g cartridge) eluting with DCM and MeOH (0-10%) and by preparative HPLC (BEH, C18) eluting with water [10 mM (NH₄)(HCO₂)] and MeCN (15-25%) to provide the title compound as a solid (37 mg, 54%). ¹H NMR (300 MHz, DMSO-d6) δ 10.85 (s, 1H), 8.87 (d, J=2.3 Hz, 1H), 8.26 (s, 1H), 8.21 (dd, J=9.0, 2.4 Hz, 1H), 7.63 (br, 2H), 7.48 (s, 1H), 7.29-7.20 (m, 2H), 6.70 (d, J=9.1 Hz, 1H), 4.31 (s, 3H), 3.89 (d, J=5.8 Hz, 2H), 3.79-3.68 (m, 4H), 2.68-2.59 (m, 1H), 1.57 (d, J=8.8 Hz, 1H). m/z (ES⁺) [M+H]⁺ 415.3. HPLC (A05) t_{R}=2.20 min.

### EXAMPLE B99

### Preparation of Compound A-285

### Step 1: N-[5-(1H-benzimidazol-2-yl)-1-methyl-pyrazol-3-yl]-6-(3-oxa-8-azabicyclo[3.2.1]octan-8-yl)pyridine-3-carboxamide

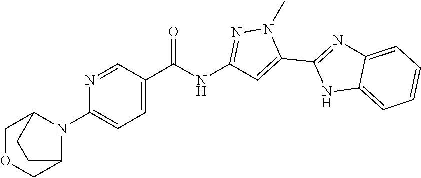 [SMILES: Cn1nc(NC(=O)c2ccc(N3C4CCC3COC4)nc2)cc1-c1nc2ccccc2[nH]1]

N-[5-(1H-benzimidazol-2-yl)-1-methyl-pyrazol-3-yl]-6-chloro-pyridine-3-carboxamide (120 mg, 0.340 mmol), 3-oxa-8-azabicyclo[3.2.1]octane hydrochloride (50.9 mg, 0.340 mmol) and DIEA (0.175 mL, 1.02 mmol) were dissolved in DMSO (3 mL). The mixture was stirred at 100° C. for 36 h. More 3-oxa-8-azabicyclo[3.2.1]octane hydrochloride (25.4 mg, 0.100 mmol) was added, and the mixture was stirred at 100° C. for 18 h. After cooling at 23° C., water (20 mL) was added, and the precipitate was filtered. The residue was purified by silica gel chromatography (24 g cartridge) eluting with DCM and MeOH (0-10%) and by preparative HPLC (BEH, C18) eluting with water [10 mM (NH₄)(HCO₂)] and MeCN (28-38%) to provide the formate salt of the title compound as a solid (27 mg, 19%). ¹H NMR (500 MHz, DMSO-d6) δ 10.83 (s, 1H), 8.80 (d, J=1.9 Hz, 1H), 8.16 (dd, J=8.9, 2.5 Hz, 1H), 7.78-7.66 (m, 1H), 7.61-7.52 (m, 1H), 7.48 (s, 1H), 7.29-7.17 (m, 2H), 6.85 (d, J=8.7 Hz, 1H), 4.65-4.56 (m, 2H), 4.30 (s, 3H), 3.62 (d, J=10.7 Hz, 2H), 3.55 (d, J=11.6 Hz, 2H), 2.02-1.95 (m, 2H), 1.94-1.89 (m, 2H). m/z (ES⁺), [M−HCO₂]⁺ 430.3. HPLC (A05) t_{R}=3.13 min.

### EXAMPLE B100

### Preparation of Compound A-286

### Step 1: tert-butyl 3-[5-[[5-(1H-benzimidazol-2-yl)-1-methyl-pyrazol-3-yl]carbamoyl]-2-pyridyl]-3,8-diazabicyclo[3.2.1]octane-8-carboxylate

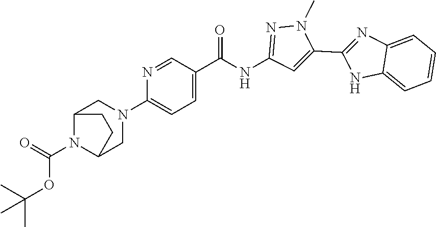 [SMILES: Cn1nc(NC(=O)c2ccc(N3CC4CCC(C3)N4C(=O)OC(C)(C)C)nc2)cc1-c1nc2ccccc2[nH]1]

N-[5-(1H-benzimidazol-2-yl)-1-methyl-pyrazol-3-yl]-6-chloro-pyridine-3-carboxamide (70 mg, 0.198 mmol), tert-butyl 3,8-diazabicyclo[3.2.1]octane-8-carboxylate (42.1 mg, 0.198 mmol) and DIEA (0.102 mL, 0.595 mmol) were dissolved in DMSO (1.75 mL). The mixture was stirred at 85° C. for 18 h. More tert-butyl 3,8-diazabicyclo[3.2.1]octane-8-carboxylate (21.1 mg, 0.100 mmol) was added, and the mixture was stirred at 85° C. for 18 h. After cooling at 23° C., water (20 mL) was added, and the precipitate was filtered. The residue was purified by silica gel chromatography (12 g cartridge) eluting with DCM and MeOH (0-10%) to provide the title compound as a solid (65 mg, 62%). ¹H NMR (300 MHz, DMSO-d6) δ 13.06 (s, 1H), 10.86 (s, 1H), 8.80 (d, J=2.4 Hz, 1H), 8.16 (dd, J=9.1, 2.5 Hz, 1H), 7.76-7.67 (m, 1H), 7.59-7.48 (m, 1H), 7.48 (s, 1H), 7.31-7.21 (m, 2H), 6.83 (d, J=9.1 Hz, 1H), 4.30 (s, 3H), 4.28-4.23 (m, 2H), 4.12 (d, J=11.7 Hz, 2H), 3.00 (d, J=10.7 Hz, 2H), 1.90-1.81 (m, 2H), 1.68-1.58 (m, 2H), 1.43 (s, 9H). m/z (ES⁺), [M+H]⁺ 529.2. HPLC (A05) t_{R}=2.56 min.

### Step 2: N-[5-(1H-benzimidazol-2-yl)-1-methyl-pyrazol-3-yl]-6-(3,8-diazabicyclo[3.2.1]octan-3-yl)pyridine-3-carboxamide

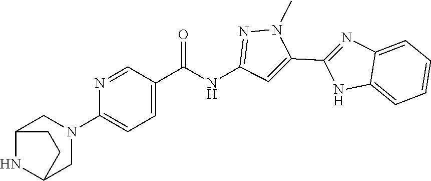 [SMILES: Cn1nc(NC(=O)c2ccc(N3CC4CCC(C3)N4)nc2)cc1-c1nc2ccccc2[nH]1]

A solution of tert-butyl 3-[5-[[5-(1H-benzimidazol-2-yl)-1-methyl-pyrazol-3-yl]carbamoyl]-2-pyridyl]-3,8-diazabicyclo[3.2.1]octane-8-carboxylate (65 mg, 0.123 mmol) in HFIP (2.46 mL) was stirred at 100° C. for 48 h. The mixture was concentrated. The residue was purified by silica gel chromatography (12 g cartridge) eluting with DCM and MeOH (0-10%) and by preparative HPLC (BEH, C18) eluting with water [10 mM (NH₄)(HCO₂)] and MeCN (29-39%) to provide the title compound as a solid (25 mg, 47%). ¹H NMR (300 MHz, DMSO-d6) δ 13.06 (s, 1H), 10.82 (s, 1H), 8.78 (d, J=2.3 Hz, 1H), 8.12 (dd, J=9.2, 2.6 Hz, 1H), 7.72 (d, J=7.7 Hz, 1H), 7.53 (d, 1H), 7.47 (s, 1H), 7.32-7.23 (m, 2H), 6.73 (d, J=9.5 Hz, 1H), 4.30 (s, 3H), 4.03-3.79 (m, 2H), 3.61-3.47 (m, 2H), 3.04-2.87 (m, 2H), 1.72-1.52 (m, 4H). m/z (ES⁺) [M+H]⁺ 429.2. HPLC (A05) t_{R}=2.35 min.

### EXAMPLE B101

### Preparation of Compound A-287

### Step 1: N-[5-(1H-benzimidazol-2-yl)-1-methyl-pyrazol-3-yl]-6-[4-[2-(2-hydroxyethoxy)acetyl]piperazin-1-yl]pyridine-3-carboxamide

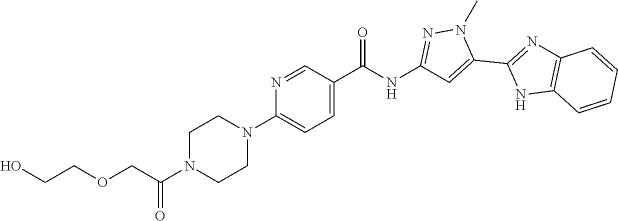 [SMILES: Cn1nc(NC(=O)c2ccc(N3CCN(C(=O)COCCO)CC3)nc2)cc1-c1nc2ccccc2[nH]1]

N-[5-(1H-benzimidazol-2-yl)-1-methyl-pyrazol-3-yl]-6-piperazin-1-yl-pyridine-3-carboxamide (Compound A-216) (100 mg, 0.248 mmol), [2-(2-hydroxyethoxy)acetyl]oxysodium (35.3 mg, 0.248 mmol) and DIEA (0.0851 mL, 0.497 mmol) were dissolved in DMF (1.20 mL) at 23° C. PyBOP (129 mg, 0.248 mmol) was added, and the mixture was stirred for 18 h. Water (20 mL) was added, and the precipitate was filtered. The residue was purified by silica gel chromatography (12 g cartridge) eluting with DCM and MeOH (0-20%) and by preparative HPLC (BEH C18 30×100) eluting with water (10 mM (NH₄)(HCO₃)) and MeCN (20-30%) to provide the formate salt of the title compound as a solid (11.6 mg, 8%). ¹H NMR (400 MHz, DMSO-d6) δ 10.89 (s, 1H), 8.82 (d, J=2.1 Hz, 1H), 8.41 (s, 1H),) 8.18 (dd, J=9.2, 2.1 Hz, 1H), 7.77-7.54 (m, 2H), 7.48 (s, 1H), 7.25 (dq, J=6.1, 3.7 Hz, 2H), 6.92 (d, J=9.1 Hz, 1H), 4.75 (s, 1H), 4.30 (s, 3H), 4.22 (s, 2H), 3.68 (dd, J=16.0, 6.8 Hz, 4H), 3.60-3.45 (m, 8H). m/z (ES⁺), [M+H]⁺ 505.4. HPLC (A05) t_{R}=1.96 min.

### EXAMPLE B102

### Preparation of Compound A-288

### Step 1: N-[5-(1H-benzimidazol-2-yl)-1-methyl-pyrazol-3-yl]-6-[(3S,5S)-3,5-dimethylpiperazin-1-yl]pyridine-3-carboxamide

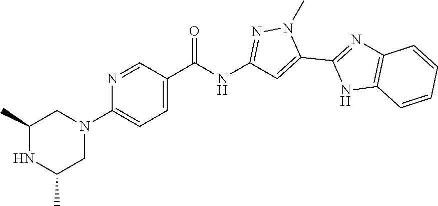 [SMILES: C[C@H]1CN(c2ccc(C(=O)Nc3cc(-c4nc5ccccc5[nH]4)n(C)n3)cn2)C[C@H](C)N1]

(2S,6S)-2,6-dimethylpiperazine dihydrochloride (40.8 mg, 0.218 mmol) was added to a solution of N-[5-(1H-benzimidazol-2-yl)-1-methyl-pyrazol-3-yl]-6-chloro-pyridine-3-carboxamide (70 mg, 0.198 mmol) and DIEA (0.136 mL, 0.794 mmol) in DMSO (1 mL). The mixture was heated to 110° C. and stirred for 18 h. The mixture was concentrated, and the residue was purified by silica gel chromatography (4 g cartridge) eluting with DCM and MeOH (0-20%) to provide the title compound as a solid (18.6 mg, 22%). ¹H NMR (400 MHz, DMSO-d6) δ 13.08 (s, 1H), 10.80 (s, 1H), 8.75 (d, J=2.3 Hz, 1H), 8.11 (dd, J=9.1, 2.3 Hz, 1H), 7.72 (d, J=8.1 Hz, 1H), 7.53 (d, J=7.8 Hz, 1H), 7.47 (s, 1H), 7.30-7.19 (m, 2H), 6.86 (d, J=9.3 Hz, 1H), 4.30 (s, 3H), 3.71 (dd, J=12.6, 3.0 Hz, 2H), 3.29 (dd, J=12.6, 6.5 Hz, 2H), 3.17-3.08 (m, 2H), 1.03 (d, J=6.4 Hz, 6H). m/z (ES⁺), [M+H]⁺ 431.4. HPLC (A05) t_{R}=1.89 min.

### EXAMPLE B103

### Preparation of Compound A-289

### Step 1: tert-butyl 4-[2-(tert-butoxycarbonylamino)acetyl]piperazine-1-carboxylate

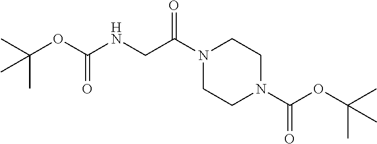 [SMILES: CC(C)(C)OC(=O)NCC(=O)N1CCN(C(=O)OC(C)(C)C)CC1]

2-(tert-butoxycarbonylamino)acetic acid (900 mg, 5.14 mmol) and HATU (1.95 g, 5.14 mmol) were dissolved in DMF (25 mL) at 0° C. DIEA (1.76 mL, 10.3 mmol) was added, and the mixture was stirred at 0° C. for 5 min. Tert-butyl piperazine-1-carboxylate (957 mg, 5.14 mmol) was added. The mixture was stirred at 23° C. for 18 h and concentrated. Water (100 mL) was added. The aq. phase was extracted with EtOAc (3×50 mL). The combined organic phase was washed with 0.1 M aq. citric acid (100 mL), brine (60 mL), dried (Na₂SO₄), filtered, and concentrated to provide the title compound as a solid (1.18 g, 67%). ¹H NMR (300 MHz, DMSO-d6) δ 6.73 (t, J=5.8 Hz, 1H), 3.76 (d, J=5.9 Hz, 2H), 3.39 (dd, J=13.8, 5.8 Hz, 4H), 3.33-3.23 (m, 4H), 1.39 (s, 9H), 1.36 (s, 9H).

### Step 2: 2-amino-1-piperazin-1-yl-ethanone dihydrochloride

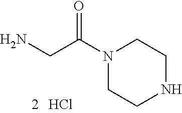

4.0 M HCl in 1,4-Dioxane (4.00 mol/L, 3.64 mL, 14.6 mmol) was added to tert-butyl 4-[2-(tert-butoxycarbonylamino)acetyl]piperazine-1-carboxylate (500 mg, 1.46 mmol). The mixture was stirred at 23° C. for 18 h. The precipitate was filtered and washed with DCM (10 mL) to provide the title compound as a solid (253 mg, 80%). ¹H NMR (400 MHz, DMSO-d6) δ 9.27 (s, 2H), 8.14 (s, 3H), 3.92 (q, J=5.9 Hz, 2H), 3.75-3.68 (m, 2H), 3.65-3.57 (m, 2H), 3.18-3.10 (m, 2H), 3.10-3.00 (m, 2H).

### Step 3: 6-[4-(2-aminoacetyl)piperazin-1-yl]-N-[5-(1H-benzimidazol-2-yl)-1-methyl-pyrazol-3-yl]pyridine-3-carboxamide

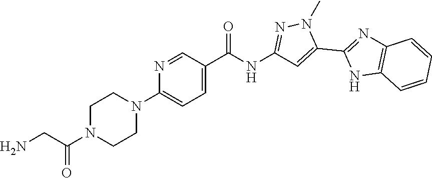 [SMILES: Cn1nc(NC(=O)c2ccc(N3CCN(C(=O)CN)CC3)nc2)cc1-c1nc2ccccc2[nH]1]

2-amino-1-piperazin-1-yl-ethanone dihydrochloride (95.6 mg, 0.442 mmol) was added to a solution of N-[5-(1H-benzimidazol-2-yl)-1-methyl-pyrazol-3-yl]-6-chloro-pyridine-3-carboxamide (120 mg, 0.340 mmol) and DIEA (0.349 mL, 2.04 mmol) in DMSO (1.20 mL). The mixture was heated to 110° C. and stirred for 18 h. The mixture was concentrated. The residue was purified by reverse phase chromatography (C18, 14 g cartridge) eluting with water (10 mM (NH₄)(HCO₃)) and MeCN (5-100%) and by prep HPLC (BEH C18 30×150) eluting with water (10 mM (NH₄)(HCO₃)) and MeCN (23-33%) to provide the title compound as a solid (32.5 mg, 21%). ¹H NMR (400 MHz, DMSO-d6) δ 10.86 (s, 1H), 8.82 (d, J=2.3 Hz, 1H), 8.18 (dd, J=9.0, 2.4 Hz, 1H), 7.76-7.51 (m, 2H), 7.47 (s, 1H), 7.25 (dd, J=5.8, 2.5 Hz, 2H), 6.91 (d, J=9.1 Hz, 1H), 4.30 (s, 3H), 3.74-3.63 (m, 4H), 3.62-3.54 (m, 2H), 3.54-3.46 (m, 2H), 3.39 (s, 2H), 3.34-3.12 (m, 2H). m/z (ES⁺), [M+H]⁺ 460.3. HPLC (A05) t_{R}=1.84 min.

### EXAMPLE B104

### Preparation of Compound A-291

### Step 1: tert-butyl N-[2-[(2-methyl-5-nitro-pyrazole-3-carbonyl)amino]-5-morpholino-phenyl]carbamate

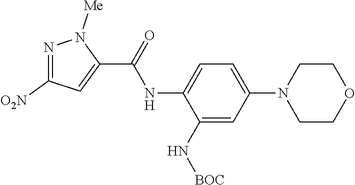 [SMILES: Cn1nc([N+](=O)[O-])cc1C(=O)Nc1ccc(N2CCOCC2)cc1NC(=O)OC(C)(C)C]

2-methyl-5-nitro-pyrazole-3-carboxylic acid (265 mg, 1.55 mmol) and HATU (589 mg, 1.55 mmol) were dissolved in DMF (5 mL) at 0° C. DIEA (0.482 mL, 2.82 mmol) was added, and the mixture was stirred at 0° C. for 5 min. Tert-butyl N-(2-amino-5-morpholino-phenyl)carbamate (Compound A-270, Step 2) (486 mg, 1.41 mmol) in DMF (5 mL) was added. The mixture was stirred at 23° C. for 6 h and concentrated. The residue was purified by silica gel chromatography (40 g cartridge) eluting with DCM and MeOH (0-10%) to provide the title compound as a solid (725 mg, 80% purity, 92%). ¹H NMR (300 MHz, DMSO-d6) δ 9.83 (s, 1H), 8.62 (s, 1H), 7.73 (s, 1H), 7.28 (d, J=2.5 Hz, 1H), 7.23 (d, J=8.8 Hz, 1H), 6.73 (dd, J=8.9, 2.7 Hz, 1H), 4.18 (s, 3H), 3.78-3.72 (m, 4H), 3.11-3.06 (m, 4H), 1.45 (s, 9H). m/z (ES⁺), [M+H]⁺ 447.3. HPLC (A05) t_{R}=2.33 min.

### Step 2: N-(2-amino-4-morpholino-phenyl)-2-methyl-5-nitro-pyrazole-3-carboxamide

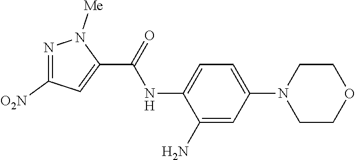 [SMILES: Cn1nc([N+](=O)[O-])cc1C(=O)Nc1ccc(N2CCOCC2)cc1N]

A solution of tert-butyl N-[2-[(2-methyl-5-nitro-pyrazole-3-carbonyl)amino]-5-morpholino-phenyl]carbamate (725 mg, 1.38 mmol) in HFIP (27 mL) was stirred at 100° C. for 4 h. After cooling to 23° C., the mixture was concentrated to provide the title compound as a solid (600 mg, 75% purity, 94%). ¹H NMR (300 MHz, DMSO-d6) δ 9.83 (s, 1H), 8.04 (d, J=7.5 Hz, 2H), 7.82 (s, 1H), 7.01 (d, J=8.6 Hz, 1H), 6.40 (s, 1H), 6.35 (d, J=8.4 Hz, 1H), 4.19 (s, 3H), 3.95-3.60 (m, 4H), 3.24-2.94 (m, 4H). m/z (ES⁺) [M+H]⁺ 347.1. HPLC (A05) t_{R}=1.98 min.

### Step 3: 4-[2-(2-methyl-5-nitro-pyrazol-3-yl)-1H-benzimidazol-5-yl]morpholine

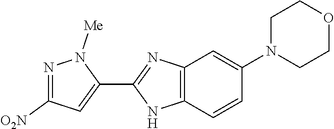 [SMILES: Cn1nc([N+](=O)[O-])cc1-c1nc2cc(N3CCOCC3)ccc2[nH]1]

A solution of N-(2-amino-4-morpholino-phenyl)-2-methyl-5-nitro-pyrazole-3-carboxamide (700 mg, 1.72 mmol) in AcOH (10 mL) was stirred at 65° C. for 18 h. The mixture was concentrated. The residue was purified by silica gel chromatography (40 g cartridge) eluting with DCM and MeOH (0-10%) to provide the title compound as a solid (285 mg, 51%). ¹H NMR (300 MHz, DMSO-d6) δ 12.94 (s, 1H), 7.67 (s, 1H), 7.63-7.51 (m, 1H), 7.13-6.96 (m, 2H), 4.43 (s, 3H), 3.84-3.72 (m, 4H), 3.17-3.10 (m, 4H). m/z (ES⁺) [M+H]⁺ 324.3. HPLC (A05) t_{R}=2.05 min.

### Step 4: 1-methyl-5-(5-morpholino-1H-benzimidazol-2-yl)pyrazol-3-amine

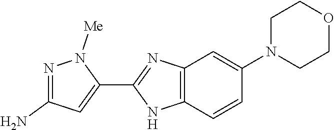 [SMILES: Cn1nc(N)cc1-c1nc2cc(N3CCOCC3)ccc2[nH]1]

4-[2-(2-methyl-5-nitro-pyrazol-3-yl)-1H-benzimidazol-5-yl]morpholine (285 mg, 0.868 mmol) was dissolved in MeOH (43 mL) and transferred in a flask purged with nitrogen containing Pd/C (10.0%, 148 mg, 0.139 mmol). The mixture was stirred at 23° C. for 18 h under an atmosphere of hydrogen. The flask was purged with nitrogen. The solution was filtered on celite, washed with MeOH (80 mL), and the filtrate was concentrated to provide the title compound as a solid (218 mg, 84%). ¹H NMR (300 MHz, DMSO-d6) δ 12.66-12.24 (m, 1H), 7.45 (d, J=8.6 Hz, 1H), 7.00-6.93 (m, 2H), 6.07 (s, 1H), 4.75 (s, 2H), 4.06 (s, 3H), 3.79-3.73 (m, 4H), 3.12-3.05 (m, 4H). m/z (ES⁺), [M+H]⁺ 298.9. HPLC (A05) t_{R}=1.75 min.

### Step 5: 6-chloro-N-[1-methyl-5-(5-morpholino-1H-benzimidazol-2-yl)pyrazol-3-yl]pyridine-3-carboxamide

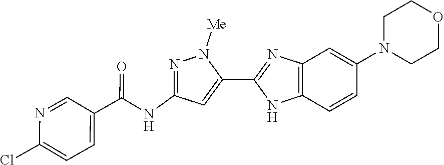 [SMILES: Cn1nc(NC(=O)c2ccc(Cl)nc2)cc1-c1nc2cc(N3CCOCC3)ccc2[nH]1]

6-chloropyridine-3-carboxylic acid (58.1 mg, 0.369 mmol) and HATU (140 mg, 0.369 mmol) were dissolved in DMF (2.24 mL) at 0° C. DIEA (0.115 mL, 0.670 mmol) was added, and the mixture was stirred at 0° C. for 5 min. 1-methyl-5-(5-morpholino-1H-benzimidazol-2-yl)pyrazol-3-amine (100 mg, 0.335 mmol) was added. The mixture was stirred at 23° C. for 18 h. The mixture was concentrated, and the residue was purified by silica gel chromatography (12 g cartridge) eluting with DCM and MeOH (0-10%) to provide the title compound as a solid (135 mg, 93% purity, 86%). ¹H NMR (300 MHz, DMSO-d6) δ 12.81 [12.87] (s, 1H), 11.38 (s, 1H), 9.00 (d, J=2.3 Hz, 1H), 8.41 (dd, J=8.4, 2.5 Hz, 1H), 7.70 (d, J=8.4 Hz, 1H), 7.57 [7.40] (d, J=8.8 Hz, 1H), 7.54-7.34 [7.21-7.17] (m, 1H), 7.16-6.94 [7.06] (d, J=9.0 Hz, 1H), 6.98-6.83 (m, 1H), 4.28 [4.30] (s, 3H), 3.85-3.58 (m, 4H), 3.14-3.05 (m, 4H). m/z (ES⁺), [M+H]⁺ 438.5. HPLC (A05) t_{R}=2.05 min.

### Step 6: 6-[(3R,5S)-3,5-dimethylpiperazin-1-yl]-N-[1-methyl-5-(5-morpholino-1H-benzimidazol-2-yl)pyrazol-3-yl]pyridine-3-carboxamide

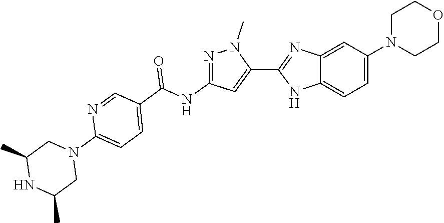 [SMILES: C[C@@H]1CN(c2ccc(C(=O)Nc3cc(-c4nc5cc(N6CCOCC6)ccc5[nH]4)n(C)n3)cn2)C[C@H](C)N1]

6-chloro-N-[1-methyl-5-(5-morpholino-1H-benzimidazol-2-yl)pyrazol-3-yl]pyridine-3-carboxamide (93% pure, 135 mg, 0.287 mmol), (2R,6S)-2,6-dimethylpiperazine (36 mg, 0.315 mmol) and DIEA (0.0982 mL, 0.573 mmol) were dissolved in DMSO (2.08 mL). The mixture was stirred at 110° C. for 24 h and concentrated. The residue was purified by silica gel chromatography (12 g cartridge) eluting with DCM and MeOH (0-10%) and by preparative HPLC (BEH, C18) eluting with water [10 mM (NH₄)(HCO₂)] and MeCN (18-28%) to provide the title compound as a solid (50 mg, 34%). ¹H NMR (400 MHz, DMSO-d6) δ 12.78 [12.84] (s, 1H), 10.79 (s, 1H), 8.77 (d, J=2.3 Hz, 1H), 8.12 (d, J=8.9 Hz, 1H), 7.56 [7.39-7.34] (d, J=8.7 Hz, 1H), 7.41 (s, 1H), 7.09-7.02 [7.22-7.14] (m, 1H), 6.95-6.83 [7.02-6.95] (m, 2H), 4.33-4.28 (m, 2H), 4.26 [4.28] (s, 3H), 3.84-3.70 (m, 4H), 3.18-2.99 (m, 4H), 2.81-2.61 (m, 2H), 2.41-2.25 (m, 2H), 1.03 (d, J=6.2 Hz, 6H). m/z (ES⁺), [M+H]⁺ 516.3. HPLC (A05) t_{R}=2.17 min.

### EXAMPLE B105

### Preparation of Compound A-290

### Step 1: 3-chloro-4-methoxy-N-[1-methyl-5-(5-morpholino-1H-benzimidazol-2-yl)pyrazol-3-yl]benzamide

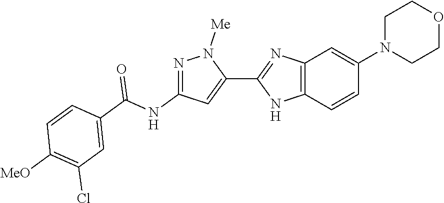 [SMILES: COc1ccc(C(=O)Nc2cc(-c3nc4cc(N5CCOCC5)ccc4[nH]3)n(C)n2)cc1Cl]

3-chloro-4-methoxy-benzoic acid (34.4 mg, 0.184 mmol) and HATU (70.1 mg, 0.184 mmol) were dissolved in DMF (1.12 mL) at 0° C. DIEA (0.0574 mL, 0.335 mmol) was added, and the mixture was stirred at 0° C. for 5 min. 1-methyl-5-(5-morpholino-1H-benzimidazol-2-yl)pyrazol-3-amine (Compound A-296, Step 4) (50 mg, 0.168 mmol) was added. The mixture was stirred at 23° C. for 18 h. The mixture was concentrated. Water (10 mL) was added. The aq. phase was extracted with EtOAc (3×15 mL), and the combined organic phases were washed with brine (10 mL), dried (Na₂SO₄), filtered, and concentrated. The residue was purified by silica gel chromatography (12 g cartridge) eluting with DCM and MeOH (0-10%) and by preparative HPLC (BEH, C18) eluting with water [10 mM (NH₄)(HCO₂)] and MeCN (33-43%) to provide the title compound as a solid (38 mg, 49%). ¹H NMR (300 MHz, DMSO-d6) δ 12.96-12.56 (m, 1H), 11.02 (s, 1H), 8.16 (d, J=2.2 Hz, 1H), 8.07 (dd, J=8.7, 2.2 Hz, 1H), 7.64-7.51 (m, 1H), 7.42 (s, 1H), 7.28 (d, J=8.8 Hz, 1H), 7.05-6.97 (m, 1H), 6.96-6.89 [7.19-7.12] (m, 1H), 4.28 (s, 3H), 3.95 (s, 3H), 3.84-3.70 (m, 4H), 3.20-3.01 (m, 4H). m/z (ES⁺), [M+H]⁺ 467.2. HPLC (A05) t_{R}=3.50 min.

### EXAMPLE B106

### Preparation of Compound A-292, A-293, A-294, and A-295

### Step 1: N-[5-(1H-benzimidazol-2-yl)-1-methyl-pyrazol-3-yl]-6-[2-(hydroxymethyl)-6-methyl-morpholin-4-yl]pyridine-3-carboxamide

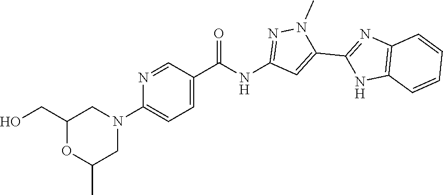 [SMILES: CC1CN(c2ccc(C(=O)Nc3cc(-c4nc5ccccc5[nH]4)n(C)n3)cn2)CC(CO)O1]

(6-methylmorpholin-2-yl)methanol (28.6 mg, 0.218 mmol) was added to a solution of N-[5-(1H-benzimidazol-2-yl)-1-methyl-pyrazol-3-yl]-6-chloro-pyridine-3-carboxamide (70 mg, 0.198 mmol) and DIEA (0.0679 mL, 0.397 mmol) in DMSO (1 mL). The mixture was heated to 110° C. and stirred for 18 h. Water (60 mL) was added, and the aq. phase was extracted with EtOAc (3×50 mL). The combined organic phases were washed with brine (50 mL), dried (Na₂SO₄), filtered, and concentrated. The residue was purified by silica gel chromatography (12 g cartridge) eluting with DCM and MeOH (0-10%). The necessary fractions were concentrated. The residue was purified by two successive SFC purifications (ChiralPak 10×250nm) eluting with water (10 mM (NH₄)(HCO₃)) and MeOH (55%) to provide isomers 1 and 4, and by SFC (ChiralPak 10×250nm) eluting with water (10 mM (NH₄)(HCO₃)) and iPrOH (45%) to provide isomers 2 and 3.

Compound A-292: (16.9 mg, 19%, 99% e.e., 1.0 formate salt); ¹H NMR (400 MHz, DMSO-d6) δ 10.86 (s, 1H), 8.79 (d, J=2.4 Hz, 1H), 8.39 (s, 1H), 8.16 (dd, J=9.0, 2.2 Hz, 1H), 7.77-7.50 (m, 2H), 7.48 (s, 1H), 7.30-7.20 (m, 2H), 6.86 (d, J=9.0 Hz, 1H), 4.77 (s, 1H), 4.30 (s, 3H), 4.06-3.94 (m, 1H), 3.83 (dd, J=12.7, 3.0 Hz, 2H), 3.74 (dd, J=13.0, 3.5 Hz, 1H), 3.57-3.46 (m, 3H), 3.27 (dd, J=13.0, 7.3 Hz, 1H), 1.15 (d, J=6.3 Hz, 3H); m/z (ES⁺), [M−HCO₂]⁺ 448.3; HPLC (A05) t_{R}=2.02 min.

Compound A-293: (5.4 mg, 5%, 93% e.e., 0.4 formate salt) ¹H NMR (500 MHz, DMSO-d6) δ 10.84 (s, 1H), 8.81-8.78 (m, 1H), 8.16 (dd, J=9.0, 2.5 Hz, 1H), 7.77-7.62 (m, 1H), 7.62-7.50 (m, 1H), 7.47 (s, 1H), 7.25 (d, J=3.3 Hz, 2H), 6.86 (d, J=9.0 Hz, 1H), 4.74 (s, 1H), 4.30 (s, 3H), 3.99 (tt, J=9.7, 4.8 Hz, 1H), 3.82 (dt, J=8.2, 3.2 Hz, 2H), 3.74 (dd, J=13.1, 3.6 Hz, 1H), 3.56-3.44 (m, 3H), 3.27 (dd, J=13.0, 7.1 Hz, 1H), 1.15 (d, J=6.3 Hz, 3H); m/z (ES⁺), [M−HCO₂]⁺ 448.3; HPLC (A05) t_{R}=2.02 min.

Compound A-294: (5.9 mg, 7%, 98% e.e., 0.5 formate) ¹H NMR (500 MHz, DMSO-d6) δ 10.79 (s, 1H), 8.74 (d, J=2.4 Hz, 1H), 8.11 (dd, J=9.0, 2.5 Hz, 1H), 7.68-7.43 (m, 2H), 7.40 (s, 1H), 7.18 (d, J=3.8 Hz, 2H), 6.83 (d, J=9.0 Hz, 1H), 4.75 (s, 1H), 4.34 (d, J=12.5 Hz, 1H), 4.26-4.19 (m, 4H), 3.54 (dtt, J=12.4, 6.1, 3.0 Hz, 1H), 3.49-3.41 (m, 2H), 3.41-3.34 (m, 1H), 2.52 (dd, J=13.0, 10.4 Hz, 1H), 2.50-2.44 (m, 1H), 1.12 (d, J=6.2 Hz, 3H); m/z (ES⁺), [M−HCO₂]⁺ 448.3; HPLC (A05) t_{R}=2.05 min.

Compound A-295: (13.5 mg, 14%, 99% e.e., 1.0 formate): ¹H NMR (400 MHz, DMSO-d6) δ 10.81 (s, 1H), 8.74 (d, J=2.2 Hz, 1H), 8.31 (s, 1H), 8.10 (dd, J=9.0, 2.2 Hz, 1H), 7.67-7.45 (m, 2H), 7.40 (s, 1H), 7.18 (dd, J=5.9, 2.9 Hz, 2H), 6.83 (d, J=9.1 Hz, 1H), 4.82 (s, 1H), 4.34 (d, J=12.6 Hz, 1H), 4.23 (s, 4H), 3.57-3.50 (m, 1H), 3.48-3.42 (m, 2H), 3.37 (dd, J=8.4, 4.0 Hz, 1H), 2.55-2.47 (m, 2H), 1.11 (d, J=6.1 Hz, 3H); m/z (ES⁺), [M−HCO₂]⁺ 448.3; HPLC (A05) t_{R}=2.05 min.

### Embodiments

The following non-limiting embodiments provide illustrative examples of the invention, but do not limit the scope of the invention.

Embodiment 1A. A compound of the formula (I):

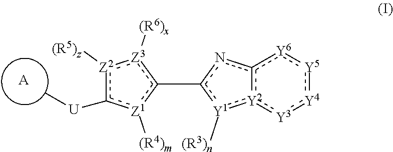

wherein:
A is an aryl or heteroaryl of the formula:

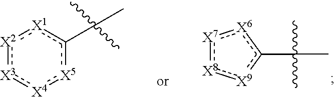

each [symbol] is independently a single bond or a double bond;
each of X¹, X², X³, X⁴, X⁵, X⁶, X⁷, X⁸, and X⁹ is independently CR¹, N, NR¹, O, or S;
Y¹ is C, N, or O wherein, (i) when Y¹ is O, n is 0; (ii) when Y′ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, or O; wherein (i) when Z¹ is O, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is a bond or a linker moiety;
each R¹ is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R² is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R^{15,} and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently H or alkyl; and
each q is independently 1, 2, 3, 4, 5, or 6,

or a pharmaceutically-acceptable salt or a tautomer thereof,

provided that, when Z¹ is C; Z² and Z³ are each N; Y¹ is N; Y² is C; U is —C(═O)—N(R)— wherein R is hydrogen or C₁-C₃ alkyl; A is phenyl or 4-pyridyl and R¹ is hydrogen, C₁-C₃ alkoxy, chlorine (Cl), fluorine (F), C₁-C₆ alkyl, —CF₃, —OH, amine, alkylamine, amide, or heterocyclic amine, then compounds of formula (I) wherein
(i) R² is hydrogen, Cl, F, C₁-C₃ alkoxy, trifluoromethoxy, trifluoromethyl or C₁-C₆ alkyl or C₃-C₁₄ aryl;
(ii) R³ is hydrogen or C₁-C₃ alkyl;
(iii) R⁴ is hydrogen;
(iv) R⁵, if present, is hydrogen or C₁-C₃ alkyl; and
(v) R⁶, if present, is hydrogen or C₁-C₃ alkyl;

are excluded.

Embodiment 2A. The compound of embodiment 1A, wherein A is

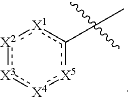

Embodiment 3A. The compound of embodiment 1A or 2A, wherein each of X¹, X², X³, X⁴, and X⁵ is independently CR¹, N or NR¹.

Embodiment 4A. The compound of any one of embodiments 1A-3A, wherein each of X¹, X², X³, X⁴, and X⁵ is CR¹.

Embodiment 5A. The compound of any one of embodiments 1A-3A, wherein one of X¹, X², X³, X⁴, and X⁵ is N or NR¹ and the others are CR¹.

Embodiment 6A. The compound of any one of embodiments 1A-3A, wherein two of X¹, X², X³, X⁴, and X⁵ are N or NR¹ and the others are CR¹.

Embodiment 7A. The compound of any one of embodiments 1A-3A, wherein A is phenyl, pyridyl or pyrimidinyl.

Embodiment 8A. The compound of embodiment 7A, wherein A is

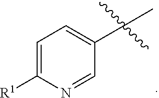

Embodiment 9A. The compound of any one of embodiments 1A-8A, wherein Y¹ is N and n is 1.

Embodiment 10A. The compound of any one of embodiments 1A-9A, wherein Y² is C.

Embodiment 11A. The compound of any one of embodiments 1A-10A, wherein each of Y³, Y⁴, Y⁵, or Y⁶ is independently CR².

Embodiment 12A. The compound of any one of embodiments 1A-8A, wherein Y¹ is C and Y² is N.

Embodiment 13A. The compound of any one of embodiments 1A-12A, wherein Z¹ is C.

Embodiment 14A. The compound of any one of embodiments 1A-13A, wherein Z² is N and z is 0.

Embodiment 15A. The compound of any one of embodiments 1A-14A, wherein Z³ is N and x is 1.

Embodiment 16A. The compound of any one of embodiments 1A-15A, wherein U is a bond, alkylene, or -L-alkylene-L′- wherein L and L′ are each independently O, N, S, amide, sulfonamide, or urea.

Embodiment 17A. The compound of embodiment 16A, wherein U is a bond, —N(R^{7a})—C(═O)—; —C(═O)—N(R^{7a})—, —N(R^{7a})—S(═O)₂—, —S(═O)₂—N(R^{7a})—, —N(R^{7a})—C(═O)N(R^{7b})—, —(CH₂)ₐ—O—, or —O—(CH₂)_{b}—, wherein
each of R^{7a} and R^{7b} is independently alkyl, alkenyl, alkynyl, cycloalkyl, aryl, arylalkyl, alkylaryl, heterocyclyl, or heteroaryl, each of which is independently unsubstituted or substituted, or hydrogen;
each a is independently 0, 1, 2, 3, 4, 5, or 6; and
each b is independently 0, 1, 2, 3, 4, 5, or 6.

Embodiment 18A. The compound of embodiment 17A, wherein U is —C(═O)—N(R^{7a})—, wherein R^{7a} is hydrogen or C₁-C₆ alkyl.

Embodiment 19A. The compound of any one of embodiments 1A-18A, wherein each R¹ is independently hydrogen, halogen, C₁-C₆ alkyl C₁-C₆ alkoxy, C₁-C₆ haloalkoxy, —OH, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵ or —S(CH₂)_{q}R¹⁶.

Embodiment 20A. The compound of embodiment 19A, wherein each R¹ is independently hydrogen, Cl, Br, CN, —OCH₃, —SCH₃, —OCF₃, —S(O)₂CH₃, —CH₃, —O(CH₂)₂OCH₃, —O(CH₂)₃OCH₃, —O(CH₂)₂morpholinyl, —OCH₂COOH, —OCH₂C(═O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OH, —NHC(CH₃)—CH₂OCH₃, or —SCH₂CH₂OCH₃.

Embodiment 21A. The compound of embodiment 20A, wherein each R¹ is independently hydrogen, Br, CN, —SCH₃, —S(O)₂CH₃, —O(CH₂)₂OCH₃, —O(CH₂)₃OCH₃, —O(CH₂)₂morpholinyl, —OCH₂COOH, —OCH₂C(═O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OH, —NHC(CH₃)—CH₂OCH₃, or —SCH₂CH₂OCH₃.

Embodiment 22A. The compound of any one of embodiments 1A-18A, wherein an R¹ is NR⁹R¹⁰, wherein R⁹ and R¹⁰ together with the nitrogen to which R⁹ and R¹⁰ are attached form an unsubstituted or substituted heterocyclic ring.

Embodiment 23A. The compound of embodiment 22A, wherein NR⁹R¹⁰ is a 4-, 5-, 6-, or 7-membered ring, optionally containing at least one additional heteroatom, wherein the additional heteroatom is O, N or S.

Embodiment 24A. The compound of embodiment 23A, wherein NR⁹R¹⁰ is an unsubstituted piperidinyl, piperazinyl or pyrrolidinyl.

Embodiment 25A. The compound of embodiment 24A, wherein NR⁹R¹⁰ is piperidinyl, piperazinyl or pyrrolidinyl, each of which is substituted by —(CH₂)ₜOR¹⁹, wherein R¹⁹ is hydrogen, C₁-C₆ alkyl, or a N-, O-, or S-containing heterocyclyl, and t is 0, 1, 2, 3, 4, 5, or 6.

Embodiment 26A. The compound of any one of embodiments 1A-25A, wherein each R² is C₁-C₆ alkyl, C₁-C₆ haloalkyl, C₁-C₆ alkoxy, or C₁-C₆ haloalkoxy, each or which is unsubstituted or substituted, or hydrogen or halogen.

Embodiment 27A. The compound of embodiment 26A, wherein each R² is independently hydrogen, halogen or C₁-C₆ alkoxy.

Embodiment 28A. The compound of embodiment 27A, wherein each R² is independently hydrogen, F, Cl, Br, CH₃, CF₃, OCH₃, or OCF₃.

Embodiment 29A. The compound of any one of embodiments 28A, wherein R³ is C₁-C₆ alkyl, —(CH₂)_{q}COOR¹⁷, or —(CH₂)_{q}OR¹⁷, each of which is independently substituted or unsubstituted, or hydrogen or halogen.

Embodiment 30A. The compound of embodiment 29A, wherein R³ is hydrogen or —CH₂—COOH.

Embodiment 31A. The compound of any one of embodiments 1A-30A, wherein R⁴ is C₁-C₆ alkyl, —(CH₂)_{q}COOR¹⁷, or —(CH₂)_{q}OR¹⁷, each of which is independently substituted or unsubstituted, or hydrogen or halogen.

Embodiment 32A. The compound of embodiment 31A, wherein R⁴ is hydrogen.

Embodiment 33A. The compound of any one of embodiments 1A-32A, wherein z is 0 and x is 1.

Embodiment 34A. The compound of embodiment 33A, wherein R⁶ is hydrogen, CH₃, —(CH₂)₂OH, or —(CH₂)₂OCH₃.

Embodiment 35A. The compound of any one of embodiments 1A-34A, which is represented by the structure of formula (II):

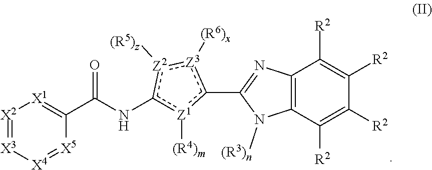

Embodiment 36A. The compound of any one of embodiments 1A-34A, which is represented by the structure of formula (III):

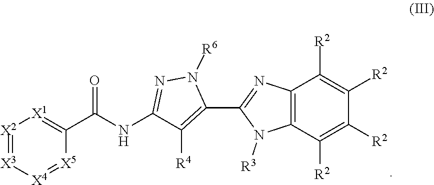

Embodiment 37A. The compound of any one of embodiments 1A-34A, which is represented by the structure of formula (IV):

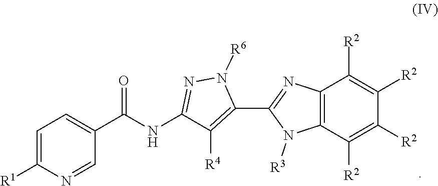

Embodiment 38A. The compound of embodiment 37A, wherein R¹ is piperidinyl, piperazinyl or pyrrolidinyl, each of which is unsubstituted or substituted.

Embodiment 39A. The compound of embodiment 37A, wherein R¹ is piperidinyl, piperazinyl or pyrrolidinyl, each of which is substituted by —(CH₂)ₜOR¹⁹, wherein R¹⁹ is hydrogen, C₁-C₆ alkyl, or N-, O-, or S-containing heterocyclyl, and t is 0, 1, 2, 3, 4, 5, or 6.

Embodiment 40A. The compound of any one of embodiments 1A-39A, which is represented by the structure of formula (V):

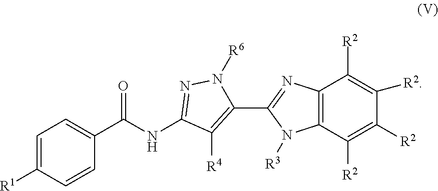

Embodiment 41A. The compound of embodiment 40A, wherein R¹ is piperidinyl, piperazinyl or pyrrolidinyl, each of which is unsubstituted or substituted.

Embodiment 42A. The compound of embodiment 40A, wherein R¹ is piperidinyl, piperazinyl or pyrrolidinyl, each of which is substituted by —(CH₂)ₜOR¹⁹, wherein R¹⁹ is hydrogen, C₁-C₆ alkyl, or N-, O-, or S-containing heterocyclyl, and t is 0, 1, 2, 3, 4, 5, or 6.

Embodiment 43A. The compound of embodiment 40A, wherein R⁶ is methyl and R¹ is piperidinyl, piperazinyl, pyrrolidinyl, or morpholinyl.

Embodiment 44A. A compound that is N-[5-(1H-benzimidazol-2-yl)-1-methyl-pyrazol-3-yl]-4-morpholino-benzamide, or a pharmaceutically-acceptable salt or a tautomer thereof.

Embodiment 45A. The compound of any one of embodiments 1A-44A which is represented by the structure of formula (VI):

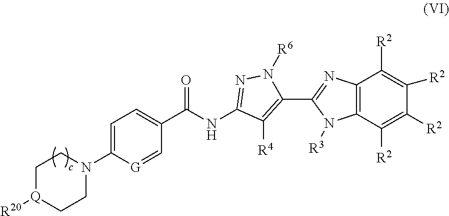

wherein
G is N or CH;
Q is N or CH;
R²⁰ is —(CH₂)ₜOR¹⁹, wherein R¹⁹ is hydrogen, a C₁-C₆ alkyl, or an N, O or S-containing heterocyclyl;
c is 0 or 1;
t is 0, 1, 2, 3, 4, 5 or 6;

or a pharmaceutically-acceptable salt or a tautomer thereof.

Embodiment 46A. The compound of embodiment 45A, which is represented by the structure of formula (VII):

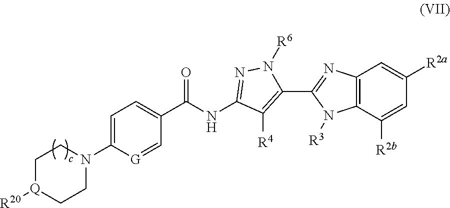

wherein R^{2a} and R^{2b} have the same meaning as R² in formula (VI).

Embodiment 47A. A pharmaceutical composition comprising a compound of any one of embodiments 1A-46A, and a pharmaceutically-acceptable excipient in a unit dosage form.

Embodiment 48A. A method of treating a condition, the method comprising administering to a subject in need thereof a therapeutically-effective amount of a compound of any one of embodiments 1A-46A.

Embodiment 49A. A method of treating a condition, the method comprising administering to a subject in need thereof a therapeutically-effective amount of a pharmaceutical composition of embodiment 47A.

Embodiment 50A. The method of embodiment 48A or embodiment 49A, wherein the condition is diabetes.

Embodiment 51A. The method of embodiment 50A, wherein the condition is insulin-dependent diabetes.

Embodiment 52A. The method of embodiment 51A, wherein the insulin-dependent diabetes is type 1 diabetes mellitus (T1DM).

Embodiment 53A. The method of embodiment 51A, wherein the insulin-dependent diabetes is diabetes secondary to pancreatectomy.

Embodiment 54A. The method of embodiment 50A, wherein the condition is non-insulin-dependent diabetes.

Embodiment 55A. The method of embodiment 50A, wherein the condition is type 2 diabetes or gestational diabetes.

Embodiment 56A. The pharmaceutical composition of embodiment 47A, for use in a method of treating a condition in a subject in need thereof.

Embodiment 57A. A compound of any one of embodiments 1A-46A, for use in a method of treating a condition in a subject in need thereof.

Embodiment 58A. The compound for use of embodiment 57A, wherein the condition is diabetes.

Embodiment 59A. The compound for use of embodiment 57A, wherein the condition is insulin-dependent diabetes,

Embodiment 60A. The compound for use of embodiment 59A, wherein the insulin-dependent diabetes is type 1 diabetes mellitus (T1DM).

Embodiment 61A. The compound for use of embodiment 59A, wherein the insulin-dependent diabetes is diabetes secondary to pancreatectomy.

Embodiment 62A. The compound for use of embodiment 57A, wherein the condition is non-insulin-dependent diabetes.

Embodiment 63A. The compound for use of embodiment 57A, wherein the condition is type 2 diabetes or gestational diabetes.

Embodiment 1. A compound of formula (Ib)

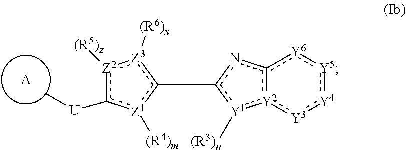

wherein:
A is an aryl or heteroaryl of the formula:

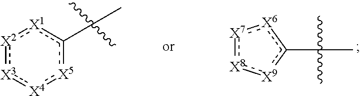

each [symbol] is independently a single bond or a double bond;
each of X¹, X², X³, X⁴, X⁵, X⁶, X⁷, X⁸, and X⁹ is independently CR¹, N, NR¹, O, or S;
Y¹ is C, N, or O wherein, (i) when Y¹ is O, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, or O; wherein (i) when Z¹ is O, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is —C(═O)—N(R^{7a})—, wherein R^{7a} is hydrogen or C₁-C₆ alkyl;
each R¹ is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen, wherein

when A is phenyl or 4-pyridyl,
(i) at least one of R¹ is independently Br, CN, —SCH₃, —S(O)₂CH₃, —O(CH₂)₂OCH₃, —O(CH₂)₃OCH₃, —O(CH₂)₂morpholinyl, —OCH₂COOH, —OCH₂C(═O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OH, —NHC(CH₃)—CH₂OCH₃, —SCH₂CH₂OCH₃; or
(ii) at least one of R¹ is NR⁹R¹⁰, wherein NR⁹R¹⁰ is piperidinyl, piperazinyl or pyrrolidinyl, each of which is substituted by —(CH₂)ₜOR¹⁹, wherein R¹⁹ is hydrogen, C₁-C₆ alkyl, or N-, O-, or S-containing heterocyclyl, and t is 0, 1, 2, 3, 4, 5, or 6;
each R² is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof.

Embodiment 2. The compound of embodiment 1, wherein each of X¹, X², X³, X⁴, and X⁵ is independently CR¹, N or NR¹.

Embodiment 3. The compound of embodiment 1 or 2, wherein each of X¹, X², X³, X⁴, and X⁵ is CR¹.

Embodiment 4. The compound of embodiment 1 or 2, wherein one of X¹, X², X³, X⁴, and X⁵ is N or NR¹ and the others are CR¹.

Embodiment 5. The compound of embodiment 1 or 2, wherein two of X¹, X², X³, X⁴, and X⁵ are N or NR¹ and the others are CR¹.

Embodiment 6. The compound of any one of embodiments 1-3, wherein A is phenyl, pyridyl or pyrimidinyl.

Embodiment 7. The compound of any one of embodiments 1, 2, 4, or 6, wherein A is

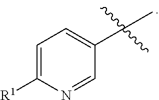

Embodiment 8. The compound of any one of embodiments 1-3 or 6, wherein A is phenyl.

Embodiment 9. The compound of any one of embodiments 1-3, 6, or 8, which is represented by the structure of formula (Vb)

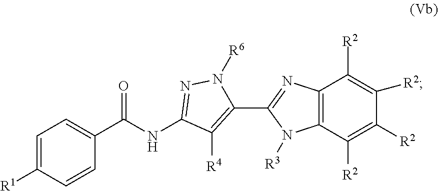

wherein:
R¹ is independently Br, CN, —SCH₃, —S(O)₂CH₃, —O(CH₂)₂OCH₃, —O(CH₂)₃OCH₃, —O(CH₂)₂morpholinyl, —OCH₂COOH, —OCH₂C(═O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OH, —NHC(CH₃)—CH₂OCH₃, —SCH₂CH₂OCH₃; or
R¹ is NR⁹R¹⁰, wherein NR⁹R¹⁰ is piperidinyl, piperazinyl or pyrrolidinyl, each of which is substituted by —(CH₂)ₜOR¹⁹, wherein R¹⁹ is hydrogen, C₁-C₆ alkyl, or N-, O-, or S-containing heterocyclyl, and t is 0, 1, 2, 3, 4, 5, or 6.

Embodiment 10. The compound of any one of embodiments 1-9, wherein R¹⁹ is a substituted C₁-C₆ alkyl.

Embodiment 11. The compound of any one of embodiments 1-10, wherein an R¹ is independently Br, CN, —SCH₃, —S(O)₂CH₃, —O(CH₂)₂OCH₃, —O(CH₂)₃OCH₃, —O(CH₂)₂morpholinyl, —OCH₂COOH, —OCH₂C(═O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OH, —NHC(CH₃)—CH₂OCH₃, or —SCH₂CH₂OCH₃.

Embodiment 12. The compound of any one of embodiments 1-10, wherein an R¹ is NR⁹R¹⁰, wherein NR⁹R¹⁰ is piperidinyl, piperazinyl, azetidinyl, pyrrolidinyl, or morpholinyl, each of which is independently substituted by —(CH₂)ₜOR¹⁹, wherein R¹⁹ is hydrogen, C₁-C₆ alkyl, or a N-, O-, or S-containing heterocyclyl, and t is 0, 1, 2, 3, 4, 5, or 6.

Embodiment 13. The compound of any one of embodiments 1-10 or 12, wherein each piperidinyl, piperazinyl, azetidinyl, pyrrolidinyl, or morpholinyl is independently substituted by an oxygen-containing heterocyclic ring.

Embodiment 14. The compound of embodiment 13, wherein the oxygen-containing heterocyclic ring is oxetanyl.

Embodiment 15. The compound of any one of embodiments 1-14, wherein each R² is hydrogen, halogen, or C₁-C₆ alkoxy.

Embodiment 16. The compound of any one of embodiments 1-15, wherein R³ is C₁-C₆ alkyl, —(CH₂)_{q}COOR¹⁷, or —(CH₂)_{q}OR¹⁷, each of which is independently substituted or unsubstituted, or hydrogen or halogen.

Embodiment 17. The compound of any one of embodiments 1-16, wherein R³ is hydrogen.

Embodiment 18. The compound of any one of embodiments 1-16, wherein R³ is —CH₂—COOH.

Embodiment 19. The compound of any one of embodiments 1-18, wherein R⁴ is C₁-C₆ alkyl, —(CH₂)_{q}COOR¹⁷, or —(CH₂)_{q}OR¹⁷, each of which is independently substituted or unsubstituted, or hydrogen or halogen.

Embodiment 20. The compound of any one of embodiments 1-19, wherein R⁴ is hydrogen.

Embodiment 21. The compound of any one of embodiments 1-20, wherein R⁶ is hydrogen, CH₃, —(CH₂)₂OH, or —(CH₂)₂OCH₃.

Embodiment 22. The compound of any one of embodiments 1-21, wherein R⁶ is hydrogen.

Embodiment 23. The compound of any one of embodiments 1-21, wherein R⁶ is CH₃.

Embodiment 24. A compound which is represented by the structure of formula (VIa):

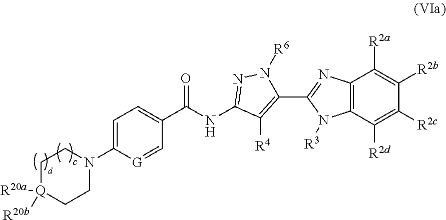

wherein
G is N or CH;
Q is N or C;
each R^{2a}, R^{2b}, R^{2c} and R^{2d} independently alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R^{20a} and R^{20b} is independently:
i) —(CH₂)ₜOR¹⁹, wherein R¹⁹ is hydrogen, a C₁-C₆ alkyl, wherein the C₁-C₆ alkyl is unsubstituted or substituted, or a N, O, or S-containing heterocyclyl;
ii) —C(O)R^{19a}, wherein R^{19a} is C₁-C₆ alkyl or —(CH₂)ₓOR²¹, wherein R²¹ is C₁-C₆ alkyl that is unsubstituted or substituted with NH₂ or OH, wherein x is 0 or 1;
iii) —NR²²R²³, wherein R²² and R²³ are independently C₁-C₆ alkyl, or R²² and R²³, together with the nitrogen to which are R²² and R²³ are attached, form an unsubstituted or substituted heterocyclic ring;
iv) —OR²⁴, wherein R²⁴ is C₁-C₆ alkyl;
v) an oxygen-containing heterocyclic ring;
vi) —C₁-C₆ alkyl, —OH, or absent; or
vii) hydrogen, provided that at least one of R^{20a} and R^{20b} is not hydrogen; or R^{20a} and R^{20b} together with Q form an oxygen-containing heterocyclic ring;
c is 0 or 1;
d is 0 or 1; and
t is 0, 1, 2, 3, 4, 5 or 6;
   or a pharmaceutically-acceptable salt or a tautomer thereof.

Embodiment 25. The compound of any one of embodiments 1-4, 6-17, 19, 20, 22, or 24, which is represented by the structure of formula (VIIb):

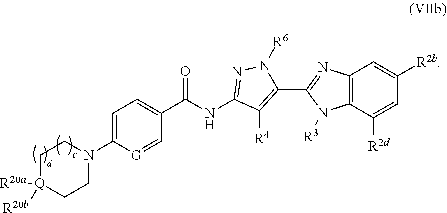

Embodiment 26. A compound of formula (Ia)

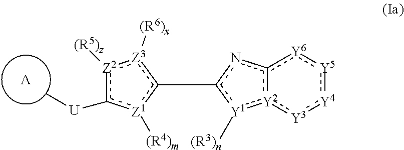

wherein:
A is

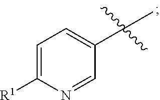

Y¹ is C, N, or O wherein, (i) when Yⁱ is O, n is 0; (ii) when Yⁱ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, or O; wherein (i) when Z¹ is O, m is 0, (ii) when Z¹ is N, m is 0 or 1, and (iii) wherein Z′ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is a bond or a linker moiety;
each R¹ is independently alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R² is independently alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof.

Embodiment 27. The compound of embodiment 26, wherein Y¹ is N and n is 1.

Embodiment 28. The compound of embodiment 26, wherein Y² is C.

Embodiment 29. The compound of embodiment 26 or 28, wherein each of Y³, Y⁴, Y⁵, or Y⁶ is independently CR².

Embodiment 30. The compound of embodiment 26, wherein Y¹ is C and Y² is N.

Embodiment 31. The compound of any one of embodiments 26-30, wherein Z¹ is C.

Embodiment 32. The compound of any one of embodiments 26-31, wherein Z² is N and z is 0.

Embodiment 33. The compound of any one of embodiments 26-32, wherein Z³ is N and x is 1.

Embodiment 34. The compound of any one of embodiments 26-33, wherein U is a bond, alkylene, an amide group, an amino group, a carbamate, a sulfoxide group, a sulfone group, a sulfonamide group, carboxyl group, an ester group, urea, or -L-alkylene-L′- wherein L and L′ are each independently O, N, S; each of which is substituted or unsubstituted.

Embodiment 35. The compound of any one of embodiments 26-34, wherein U is a bond, alkylene, an amino group, a carbamate, carboxyl group, an ester group, urea, or -L-alkylene-L′- wherein L and L′ are each independently O, N, S; each of which is substituted or unsubstituted.

Embodiment 36. The compound of any one of embodiments 26-35, wherein U is a bond, —N(R^{7a})—C(═O)—; —C(═O)—N(R^{7a})—, —N(R^{7a})—S(═O)₂—, —S(═O)₂—N(R^{7a})—, —N(R^{7a})—C(═O)N(R^{7b})—, —(CH₂)ₐ—O—, or —O—(CH₂)_{b}—, wherein
each of R^{7a} and R^{7b} is independently alkyl, alkenyl, alkynyl, cycloalkyl, aryl, arylalkyl, alkylaryl, heterocyclyl, or heteroaryl, each of which is independently unsubstituted or substituted, or hydrogen;
each a is independently 0, 1, 2, 3, 4, 5, or 6; and
each b is independently 0, 1, 2, 3, 4, 5, or 6.

Embodiment 37. The compound of any one of embodiments 26-36, wherein U is —C(═O)—N(R^{7a})—, wherein R^{7a} is hydrogen or C₁-C₆ alkyl.

Embodiment 38. The compound of any one of embodiments 26-37, wherein each R¹ is independently hydrogen, halogen, C₁-C₆ alkyl C₁-C₆ alkoxy, C₁-C₆ haloalkoxy, —OH, —CN, —NH₂, —NHR⁸, —NR⁹R¹⁰, —S(O)₂R¹², —NHC(O)R¹³, —O(CH₂)_{q}R¹⁴, —NH(CH₂)_{q}R¹⁵ or —S(CH₂)_{q}R¹⁶.

Embodiment 39. The compound of any one of embodiments 26-38, wherein each R¹ is independently alkyl, alkenyl, alkynyl, cycloalkyl, arylalkyl, alkylaryl, heterocyclyl, heteroaryl, —OR⁷, —CN, —NH₂, —NHR⁸, —NR⁹R¹⁰, —S(O)₂R¹², —NHC(O)R¹³, —O(CH₂)_{q}R¹⁴, —NH(CH₂)_{q}R¹⁵, or —S(CH₂)c₁R¹⁶, each of which is independently substituted or unsubstituted, or hydrogen.

Embodiment 40. The compound of any one of embodiments 26-39, wherein each R¹ is independently hydrogen, Cl, Br, CN, —OCH₃, —SCH₃, —OCF₃, —S(O)₂CH₃, —CH₃, —O(CH₂)₂OCH₃, —O(CH₂)₃OCH₃, azatadinyl, 2-azaspiro[3.3]heptanyl, 2-oxa-6-azaspiro[3.3]heptanyl, pyrrolidinyl, piperazinyl, piperidinyl, morpholinyl, —O(CH₂)₂morpholinyl, —OC₂H₅O, —OCH₂COOH, —OCH₂C(═O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OH, —NHC(CH₃)—CH₂OCH₃, or —SCH₂CH₂OCH₃, each of which is substituted or unsubstituted.

Embodiment 41. The compound of any one of embodiments 26-40, wherein each R¹ is independently hydrogen, Br, CN, —SCH₃, —S(O)₂CH₃, —O(CH₂)₂OCH₃, —O(CH₂)₃OCH₃, —O(CH₂)₂morpholinyl, —OCH₂COOH, —OCH₂C(═O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OH, —NHC(CH₃)—CH₂OCH₃, or —SCH₂CH₂OCH₃.

Embodiment 42. The compound of any one of embodiments 26-39, wherein an R¹ is NR⁹R¹⁰, wherein R⁹ and R¹⁰ together with the nitrogen to which R⁹ and R¹⁰ are attached form an unsubstituted or substituted heterocyclic ring.

Embodiment 43. The compound of any one of embodiments 26-39 or 42, wherein NR⁹R¹⁰ is a 4-, 5-, 6-, or 7-membered ring, optionally containing at least one additional heteroatom, wherein the additional heteroatom is O, N or S.

Embodiment 44. The compound of any one of embodiments 26-39, 42, or 43, wherein NR⁹R¹⁰ is piperidinyl, piperazinyl, azetidinyl, pyrrolidinyl, or morpholinyl, each of which is unsubstituted or substituted.

Embodiment 45. The compound of any one of embodiments 26-39 or 42-44, wherein NR⁹R¹⁰ is unsubstituted or substituted piperidinyl.

Embodiment 46. The compound of any one of embodiments 26-39 or 42-44, wherein NR⁹R¹⁰ is unsubstituted or substituted piperazinyl.

Embodiment 47. The compound of any one of embodiments 26-39 or 42-44, wherein NR⁹R¹⁰ is unsubstituted or substituted azetidinyl.

Embodiment 48. The compound of any one of embodiments 26-39 or 42-47, wherein each piperidinyl, piperazinyl, azetidinyl, pyrrolidinyl, or morpholinyl is independently substituted by —(CH₂)ₜOR¹⁹, wherein R¹⁹ is hydrogen, C₁-C₆ alkyl, or a N-, O-, or S-containing heterocyclyl, and t is 0, 1, 2, 3, 4, 5, or 6.

Embodiment 49. The compound of any one of embodiments 26-39 or 42-48, wherein each piperidinyl, piperazinyl, azetidinyl, pyrrolidinyl, or morpholinyl is independently substituted by C₁-C₆ alkyl.

Embodiment 50. The compound of any one of embodiments 26-39 or 42-48, wherein each piperidinyl, piperazinyl, azetidinyl, pyrrolidinyl, or morpholinyl is independently substituted a C₁-C₆ alkyl, wherein the C₁-C₆ alkyl may be unsubstituted or substituted.

Embodiment 51. The compound of any one of embodiments 26-39 or 42-48, wherein each piperidinyl, piperazinyl, azetidinyl, pyrrolidinyl, or morpholinyl is independently substituted —N(R²⁰)₂, wherein each R²⁰ is independently hydrogen or a C₁-C₆ alkyl.

Embodiment 52. The compound of any one of embodiments 26-39 or 42-48, wherein each piperidinyl, piperazinyl, azetidinyl, pyrrolidinyl, or morpholinyl is independently substituted by an oxygen-containing heterocyclic ring.

Embodiment 53. The compound of embodiment 52, wherein the oxygen-containing heterocyclic ring is oxetanyl.

Embodiment 54. The compound of any one of embodiments 26-39 or 42, wherein NR⁹R¹⁰ is a bicyclic ring comprising piperidinyl, piperazinyl, morpholinyl or pyrrolidinyl, each of which is substituted or unsubstituted.

Embodiment 55. The compound of any one of embodiments 26-39, 42, or 54, wherein NR⁹R¹⁰ is a fused bicyclic ring.

Embodiment 56. The compound of any one of embodiments 26-39, 42, 54, or 55, wherein NR⁹R¹⁰ is pyrazinyl fused to cyclopentanonyl.

Embodiment 57. The compound of any one of embodiments 26-39, 42, 54, or 55, wherein NR⁹R¹⁰ is pyrrolidinyl fused to tetrahydrofuranyl.

Embodiment 58. The compound of any one of embodiments 26-39, 42, or 54, wherein NR⁹R¹⁰ is a spiro bicyclic ring moiety.

Embodiment 59. The compound of embodiment 58, wherein the spiro bicyclic ring moiety is 2-oxa-6-azaspiro[3.4]octanyl, 1-oxa-8-azaspiro[4.5]decanyl, 2-azaspiro-3.3]heptanyl, 2-oxa-6-azaspiro[3.3]heptanyl, or 2-oxa-7-azaspiro[3.5]nonanyl, each of which is substituted or unsubstituted.

Embodiment 60. The compound of any one of embodiments 26-39, 42, or 54, wherein NR⁹R¹⁰ is a bridged bicyclic ring moiety.

Embodiment 61. The compound of embodiment 60, wherein the bridged bicyclic ring moiety is 3,6-diazabicyclo[3.1.1]heptanyl, 3-oxa-8-azabicycli[3.2.1]octanyl, or 3,8-diazabicyclo[3.2.1]octanyl

Embodiment 62. The compound of any one of embodiments 26-61, wherein each R² is independently C₁-C₆ alkyl, C₁-C₆ haloalkyl, C₁-C₆ alkoxy, or C₁-C₆ haloalkoxy, each or which is unsubstituted or substituted, or hydrogen or halogen.

Embodiment 63. The compound of any one of embodiments 26-62, wherein each R² is independently C₂-C₆ alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen.

Embodiment 64. The compound of any one of embodiments 26-63, wherein each R² is independently hydrogen, halogen or C₁-C₆ alkoxy.

Embodiment 65. The compound of any one of embodiments 26-63, wherein each R² is independently hydrogen, F, Cl, Br, CH₃, CF₃, OCH₃, or OCF₃.

Embodiment 66. The compound of any one of embodiments 26-65, wherein each R² is hydrogen.

Embodiment 67. The compound of any one of embodiments 26-66, wherein R³ is C₁-C₆ alkyl, —(CH₂)_{q}COOR¹⁷, or —(CH₂)_{q}OR¹⁷, each of which is independently substituted or unsubstituted, or hydrogen or halogen.

Embodiment 68. The compound of any one of embodiments 26-67, wherein R³ is hydrogen.

Embodiment 69. The compound of any one of embodiments 26-67, wherein R³ is —CH₂—COOH.

Embodiment 70. The compound of any one of embodiments 26-69, wherein R⁴ is C₁-C₆ alkyl, —(CH₂)_{q}COOR¹⁷, or —(CH₂)_{q}OR¹⁷, each of which is independently substituted or unsubstituted, or hydrogen or halogen.

Embodiment 71. The compound of any one of embodiments 26-70, wherein R⁴ is hydrogen.

Embodiment 72. The compound of any one of embodiments 26-71, wherein z is 0 and x is 1.

Embodiment 73. The compound of embodiment 72, wherein R⁶ is hydrogen, CH₃, —(CH₂)₂OH, or —(CH₂)₂OCH₃.

Embodiment 74. The compound of embodiment 73, wherein R⁶ is hydrogen.

Embodiment 75. The compound of embodiment 73, wherein R⁶ is CH₃.

Embodiment 76. The compound of any one of embodiments 26-75, which is represented by the structure of formula (IIa):

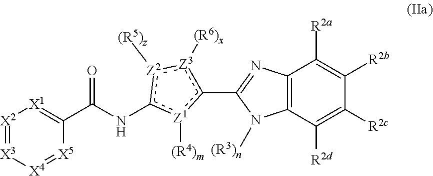

wherein each R^{2a}, R^{2b}, R^{2c} and R^{2d} independently has the same meaning as R² in formula (Ia).

Embodiment 77. The compound of any one of embodiments 26, 27, 29, or 31-76, which is represented by the structure of formula (IIIa):

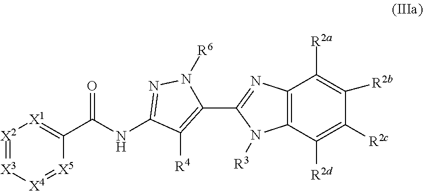

wherein each R^{2a}, R^{2b}, R^{2c} and R^{2d} independently has the same meaning as R² in formula (Ia).

Embodiment 78. The compound of any one of embodiments 26, 27, 29, or 31-77, which is represented by the structure of formula (IVa):

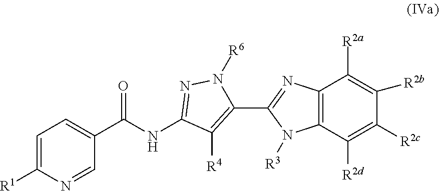

wherein each R^{2a}, R^{2b}, R^{2c} and R^{2d} independently has the same meaning as R² in formula (Ia).

Embodiment 79. The compound of any one of embodiments 76-78, wherein an R¹ is NR⁹R¹⁰, wherein R⁹ and R¹⁰ together with the nitrogen to which R⁹ and R¹⁰ are attached form an unsubstituted or substituted heterocyclic ring.

Embodiment 80. The compound of embodiment 79, wherein NR⁹R¹⁰ is a 4-, 5-, 6-, or 7-membered ring, optionally containing at least one additional heteroatom, wherein the additional heteroatom is O, N or S.

Embodiment 81. The compound of embodiment 79 or 80, wherein NR⁹R¹⁰ is piperidinyl, piperazinyl, azetidinyl, pyrrolidinyl, or morpholinyl, each of which is unsubstituted or substituted.

Embodiment 82. The compound of any one of embodiments 79-81, wherein NR⁹R¹⁰ is unsubstituted or substituted piperidinyl.

Embodiment 83. The compound of any one of embodiments 79-81, wherein NR⁹R¹⁰ is unsubstituted or substituted piperazinyl.

Embodiment 84. The compound of any one of embodiments 79-81, wherein NR⁹R¹⁰ is unsubstituted or substituted azetidinyl.

Embodiment 85. The compound of any one of embodiments 79-84, wherein each piperidinyl, piperazinyl, azetidinyl, pyrrolidinyl, or morpholinyl is independently substituted by —(CH₂)ₜOR¹⁹, wherein R¹⁹ is hydrogen, C₁-C₆ alkyl, or a N-, O-, or S-containing heterocyclyl, and t is 0, 1, 2, 3, 4, 5, or 6.

Embodiment 86. The compound of any one of embodiments 79-85, wherein each piperidinyl, piperazinyl, azetidinyl, pyrrolidinyl, or morpholinyl is independently substituted by C₁-C₆ alkyl.

Embodiment 87. The compound of any one of embodiments 79-86, wherein each piperidinyl, piperazinyl, azetidinyl, pyrrolidinyl, or morpholinyl is independently substituted —N(R²⁰)₂, wherein each R²⁰ is independently hydrogen or a C₁-C₆ alkyl.

Embodiment 88. The compound of any one of embodiments 79-87, wherein each piperidinyl, piperazinyl, azetidinyl, pyrrolidinyl, or morpholinyl is independently substituted by an oxygen-containing heterocyclic ring.

Embodiment 89. The compound of embodiment 88, wherein the oxygen-containing heterocyclic ring is oxetanyl.

Embodiment 90. A compound which is represented by the structure of formula (VIa):

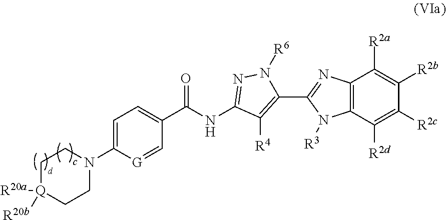

wherein
G is N;
Q is N or C;
each R^{2a}, R^{2b}, R^{2c} and R^{2d} is independently alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl;
each R^{20a} and R^{20b} is independently:
i) —(CH₂)ₜOR¹⁹, wherein R¹⁹ is hydrogen, a C₁-C₆ alkyl, wherein the C₁-C₆ alkyl is unsubstituted or substituted, or a N, O, or S-containing heterocyclyl;
ii) —C(O)R^{19a}, wherein R^{19a} is C₁-C₆ alkyl or —(CH₂)ₓOR²¹, wherein R²¹ is C₁-C₆ alkyl that is unsubstituted or substituted with NH₂ or OH, wherein x is 0 or 1;
iii) —NR²²R²³, wherein R²² and R²³ are independently C₁-C₆ alkyl;
iv) —OR²⁴, wherein R²⁴ is C₁-C₆ alkyl;
v) an oxygen-containing heterocyclic ring;
vi) —C₁-C₆ alkyl, —OH, or absent; or
vii) hydrogen, provided that at least one of R^{20a} and R^{20b} is not hydrogen; or R^{20a} and R^{20b} together with Q form an oxygen-containing heterocyclic ring;
c is 0 or 1;
d is 0 or 1;
each q is independently 1, 2, 3, 4, 5, or 6, and
t is 0, 1, 2, 3, 4, 5 or 6;
   or a pharmaceutically-acceptable salt or a tautomer thereof.

Embodiment 91. The compound of any one of embodiments 26-29, 31-44, 46, 48-81, 83, 85-90, which is represented by the structure of formula (VIIa):

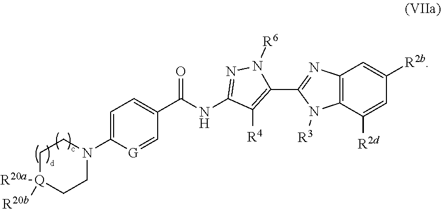

Embodiment 92. A compound of the formula (I):

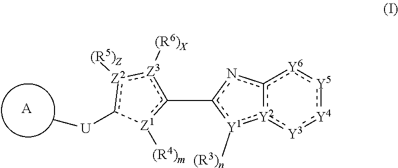

wherein:
A is an aryl or heteroaryl of the formula:

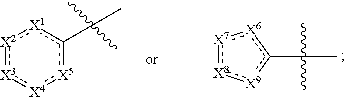

each [symbol] is independently a single bond or a double bond;
each of X¹, X², X³, X⁴, X⁵, X⁶, X⁷, X⁸, and X⁹ is independently CR¹, N, NR¹, O, or S;
Y¹ is C, N, or O wherein, (i) when Y¹ is O, n is 0; (ii) when Y¹ is N, n is 0 or 1; and (iii) when Y′ is C, n is 1;
Y² is C or N;
each of Y³, Y⁴, Y⁵, and Y⁶ is independently CR², N, NR², O, or S;
Z¹ is C, N, or O; wherein (i) when Z¹ is O, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is —C(═O)—N(R^{7a})—, wherein R^{7a} is hydrogen or C₁-C₆ alkyl;
each R¹ is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R² is independently hydrogen, halogen or C₁-C₆ alkoxy;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof,
   provided that:

when Z¹ is C, Z² and Z³ are each N; Y¹ is N, Y² is C; R^{7a} is hydrogen or C₁-C₃ alkyl; A is phenyl or 4-pyridyl; and R¹ is hydrogen, C₁-C₃ alkoxy, chlorine (Cl), fluorine (F), C₁-C₆ alkyl, —CF₃, —OH, amine, alkylamine, amide, or heterocyclic amine; then compounds of formula (I) wherein

(i) R² is hydrogen, Cl, F, or C₁-C₃ alkoxy;

(ii) R³ is hydrogen or C₁-C₃ alkyl;

(iii) R⁴ is hydrogen;

(iv) R⁵, if present, is hydrogen or C₁-C₃ alkyl; and

(v) R⁶, if present, is hydrogen or C₁-C₃ alkyl;

are excluded.

Embodiment 93. A compound of the formula (Ic):

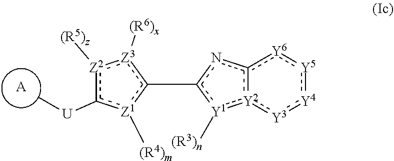

wherein:
A is an aryl or heteroaryl of the formula:

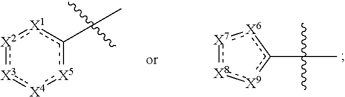

each [symbol] is independently a single bond or a double bond;
each of X¹, X², X³, X⁴, X⁵, X⁶, X⁷, X⁸, and X⁹ is independently CR¹, N, NR¹, O, or S;
Y¹ is C, N, S, or O wherein, (i) when Y¹ is O or S, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, S, or O; wherein (i) when Z¹ is O or S, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is —C(═O)—N(R^{7a})—, wherein R^{7a} is hydrogen or C₁-C₆ alkyl;
each R¹ is independently alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R² is independently hydrogen, halogen or C₁-C₆ alkoxy;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof,
   provided that:

when Z¹ is C, Z² and Z³ are each N; Y¹ is N, Y² is C; A is phenyl or 4-pyridyl; and R¹ is hydrogen, C₁-C₃ alkoxy, chlorine (Cl), fluorine (F), C₁-C₆ alkyl, —CF₃, —OH, amine, alkylamine, amide, or heterocyclic amine;

then compounds of formula (I) wherein

(i) R² is hydrogen, Cl, F, or C₁-C₃ alkoxy;

(ii) R³ is hydrogen or C₁-C₃ alkyl;

(iii) R⁴ is hydrogen;

(iv) R⁵, if present, is hydrogen or C₁-C₃ alkyl; and

(v) R⁶, if present, is hydrogen or C₁-C₃ alkyl;

are excluded.

Embodiment 94. A compound of formula (Ic′)

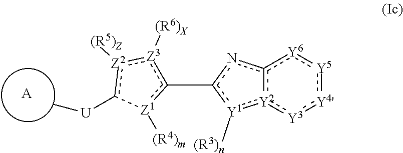

wherein:
A is

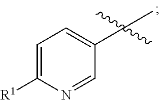

Y¹ is C, N, S, or O wherein, (i) when Y¹ is O or S, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, S, or O; wherein (i) when Z¹ is O or S, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is a bond or a linker moiety;
each R¹ is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
each R² is independently alkyl, alkenyl, alkynyl, cycloalkyl, aryl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R^{15,} and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof.

Embodiment 95. A compound of formula (Ic″)

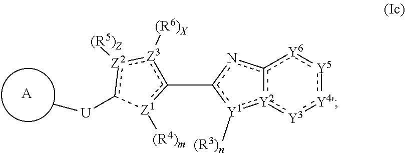

wherein:
A is an aryl or heteroaryl of the formula:

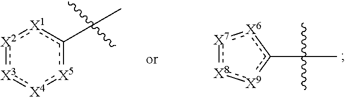

each [symbol] is independently a single bond or a double bond;
each of X¹, X², X³, X⁴, X⁵, X⁶, X⁷, X⁸, and X⁹ is independently CR¹, N, NR¹, O, or S;
Y¹ is C, N, S, or O wherein, (i) when Y¹ is O or S, n is 0; (ii) when Y¹ is N, n is 0 or 1; and (iii) when Y¹ is C, n is 1;
Y² is C or N;
each of Y³, Y⁴, Y⁵, and Y⁶ is independently CR², N, NR², O, or S;
Z¹ is C, N, S, or O; wherein (i) when Z¹ is O or S, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is —C(═O)—N(R^{7a})—, wherein R^{7a} is hydrogen or C₁-C₆ alkyl;
each R¹ is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen, wherein

when A is phenyl or 4-pyridyl,
(i) at least one of R¹ is independently Br, CN, —SCH₃, —S(O)₂CH₃, —O(CH₂)₂OCH₃, —O(CH₂)₃OCH₃, —O(CH₂)₂morpholinyl, —OCH₂COOH, —OCH₂C(═O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OH, —NHC(CH₃)—CH₂OCH₃, —SCH₂CH₂OCH₃; or
(ii) at least one of R¹ is NR⁹R¹⁰, wherein NR⁹R¹⁰ is piperidinyl, piperazinyl or pyrrolidinyl, each of which is substituted by —(CH₂)ₜOR¹⁹, wherein R¹⁹ is hydrogen, C₁-C₆ alkyl, or N-, O-, or S-containing heterocyclyl, and t is 0, 1, 2, 3, 4, 5, or 6;
each R² is independently alkyl, alkenyl, alkynyl, cycloalkyl, aryl, arylalkyl, alkylaryl, heterocyclyl, heteroaryl, —OR⁷, —CN, —NH₂, —NH₂, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, heteroaryl, —OH, —OR⁷, —NH₂, —NHR⁸, —NR⁹R¹⁰, —(CH₂)_{q}COOR¹⁷, —(CH₂)_{q}N(R¹⁷)₂, —(CH₂)_{q}OR¹⁷, or —COOR¹⁸, each of which is independently unsubstituted or substituted, or hydrogen;
each of R¹⁷ and R¹⁸ is independently hydrogen or alkyl; and
each q is independently 1, 2, 3, 4, 5, or 6,
   or a pharmaceutically-acceptable salt or a tautomer thereof.

Embodiment 96. The compound of any one of embodiments 93-95, wherein Y¹ is S.

Embodiment 97. The compound of any one of embodiments 93-96, wherein Z¹ is S.

Embodiment 98. The compound of any one of embodiments 92-97, wherein A is

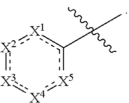

Embodiment 99. The compound of any one of embodiments 93-98, wherein each of X¹, X², X³, X⁴, and X⁵ is independently CR¹, N or NR¹.

Embodiment 100. The compound of any one of embodiments 93-99, wherein each of X¹, X², X³, X⁴, and X⁵ is CR¹.

Embodiment 101. The compound of any one of embodiments 93-99, wherein one of X¹, X², X³, X⁴, and X⁵ is N or NR¹ and the others are CR¹.

Embodiment 102. The compound of any one of embodiments 93-99, wherein two of X¹, X², X³, X⁴, and X⁵ are N or NR¹ and the others are CR¹.

Embodiment 103. The compound of any one of embodiments 93-102, wherein A is phenyl, pyridyl or pyrimidinyl.

Embodiment 104. The compound of any one of embodiments 93-99, 101, or 103, wherein A is

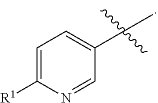

Embodiment 105. The compound of any one of embodiments 93-100 or 103, wherein A is phenyl.

Embodiment 106. The compound of any one of embodiments 92-105, wherein Y¹ is N and n is 1.

Embodiment 107. The compound of any one of embodiments 92-106, wherein Y² is C.

Embodiment 108. The compound of any one of embodiments 92-107, wherein each of Y³, Y⁴, Y⁵, or Y⁶ is independently CR².

Embodiment 109. The compound of any one of embodiments 92-105 or 108, wherein Y¹ is C and Y² is N.

Embodiment 110. The compound of any one of embodiments 92-109, wherein Z¹ is C.

Embodiment 111. The compound of any one of embodiments 92-110, wherein Z² is N and z is 0.

Embodiment 112. The compound of any one of embodiments 92-111, wherein Z³ is N and x is 1.

Embodiment 113. The compound of any one of embodiments 92-112, wherein each R¹ is independently hydrogen, halogen, C₁-C₆ alkyl C₁-C₆ alkoxy, C₁-C₆ haloalkoxy, —OH, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵ or —S(CH₂)_{q}R¹⁶.

Embodiment 114. The compound of any one of embodiments 92-113, wherein each R¹ is independently alkyl, alkenyl, alkynyl, cycloalkyl, arylalkyl, alkylaryl, heterocyclyl, heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, each of which is independently substituted or unsubstituted, or hydrogen.

Embodiment 115. The compound of any one of embodiments 92-112 or 114, wherein each R¹ is independently hydrogen, Cl, Br, CN, —OCH₃, —SCH₃, —OCF₃, —S(O)₂CH₃, —CH₃, —O(CH₂)₂OCH₃, —O(CH₂)₃OCH₃, azatadinyl, 2-azaspiro[3.3]heptanyl, 2-oxa-6-azaspiro[3.3]heptanyl, pyrrolidinyl, piperazinyl, piperidinyl, morpholinyl, —O(CH₂)₂morpholinyl, —OC₂H₅O, —OCH₂COOH, —OCH₂C(═O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OH, —NHC(CH₃)—CH₂OCH₃, or —SCH₂CH₂OCH₃, each of which is substituted or unsubstituted.

Embodiment 116. The compound of any one of embodiments 92-112, 114, or 115, wherein each R¹ is independently hydrogen, Br, CN, —SCH₃, —S(O)₂CH₃, —O(CH₂)₂OCH₃, —O(CH₂)₃OCH₃, —O(CH₂)₂morpholinyl, —OCH₂COOH, —OCH₂C(═O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OH, —NHC(CH₃)—CH₂OCH₃, or —SCH₂CH₂OCH₃.

Embodiment 117. The compound of any one of embodiments 92-114, wherein an R¹ is NR⁹R¹⁰, wherein R⁹ and R¹⁰ together with the nitrogen to which R⁹ and R¹⁰ are attached form an unsubstituted or substituted heterocyclic ring.

Embodiment 118. The compound of any one of embodiments 92-114 or 117, wherein NR⁹R¹⁰ is a 4-, 5-, 6-, or 7-membered ring, optionally containing at least one additional heteroatom, wherein the additional heteroatom is O, N or S.

Embodiment 119. The compound of any one of embodiments 92-114, 117, or 118, wherein NR⁹R¹⁰ is piperidinyl, piperazinyl, azetidinyl, pyrrolidinyl, or morpholinyl, each of which is unsubstituted or substituted.

Embodiment 120. The compound of any one of embodiments 92-114, 117-119, wherein NR⁹R¹⁰ is unsubstituted or substituted piperidinyl.

Embodiment 121. The compound of any one of embodiments 92-114, 117-119, wherein NR⁹R¹⁰ is unsubstituted or substituted piperazinyl.

Embodiment 122. The compound of any one of embodiments 92-114, 117-119, wherein NR⁹R¹⁰ is unsubstituted or substituted azetidinyl.

Embodiment 123. The compound of any one of embodiments 92-114, 117-122, wherein each piperidinyl, piperazinyl, azetidinyl, pyrrolidinyl, or morpholinyl is independently substituted by —(CH₂)ₜOR¹⁹, wherein R¹⁹ is hydrogen, C₁-C₆ alkyl, or a N-, O-, or S-containing heterocyclyl, and t is 0, 1, 2, 3, 4, 5, or 6.

Embodiment 124. The compound of any one of embodiments 92-114, 117-123, wherein each piperidinyl, piperazinyl, azetidinyl, pyrrolidinyl, or morpholinyl is independently substituted by C₁-C₆ alkyl.

Embodiment 125. The compound of any one of embodiments 92-114, 117-124, wherein each piperidinyl, piperazinyl, azetidinyl, pyrrolidinyl, or morpholinyl is independently substituted with substituted C₁-C₆alkyl.

Embodiment 126. The compound of any one of embodiments 92-114, 117-125, wherein each piperidinyl, piperazinyl, azetidinyl, pyrrolidinyl, or morpholinyl is independently substituted —N(R²⁰)₂, wherein each R²⁰ is independently hydrogen or a C₁-C₆ alkyl.

Embodiment 127. The compound of any one of embodiments 92-114, 117-126, wherein each piperidinyl, piperazinyl, azetidinyl, pyrrolidinyl, or morpholinyl is independently substituted by an oxygen-containing heterocyclic ring.

Embodiment 128. The compound of embodiment 127, wherein the oxygen-containing heterocyclic ring is oxetanyl.

Embodiment 129. The compound of any one of embodiments 92-114 or 117, wherein NR⁹R¹⁰ is a bicyclic ring comprising piperidinyl, piperazinyl, morpholinyl or pyrrolidinyl, each of which is substituted or unsubstituted.

Embodiment 130. The compound of any one of embodiments 92-114, 117, or 129, wherein NR⁹R¹⁰ is a fused bicyclic ring.

Embodiment 131. The compound of embodiment 129 or 130, wherein NR⁹R¹⁰ is pyrazinyl fused to cyclopentanonyl.

Embodiment 132. The compound of embodiment 129 or 130, wherein NR⁹R¹⁰ is pyrrolidinyl fused to tetrahydrofuranyl.

Embodiment 133. The compound of embodiment 129 or 130, wherein NR⁹R¹⁰ is a spiro bicyclic ring moiety.

Embodiment 134. The compound of embodiment 133, wherein the spiro bicyclic ring moiety is 2-oxa-6-azaspiro[3.4]octanyl, 1-oxa-8-azaspiro[4.5]decanyl, 2-azaspiro-3.3]heptanyl, 2-oxa-6-azaspiro[3.3]heptanyl, or 2-oxa-7-azaspiro[3.5]nonanyl, each of which is substituted or unsubstituted.

Embodiment 135. The compound of any one of embodiments 92-114, 117, or 129, wherein NR⁹R¹⁰ is a bridged bicyclic ring moiety.

Embodiment 136. The compound of embodiment 135, wherein the bridged bicyclic ring moiety is 3,6-diazabicyclo[3.1.1]heptanyl, 3-oxa-8-azabicycli[3.2.1]octanyl, or 3,8-diazabicyclo[3.2.1]octanyl

Embodiment 137. The compound of any one of embodiments 92-136, wherein each R² is hydrogen, halogen, or C₁-C₆ alkoxy.

Embodiment 138. The compound of any one of embodiments 92-137, wherein R³ is C₁-C₆ alkyl, —(CH₂)_{q}COOR¹⁷, or —(CH₂)_{q}OR¹⁷, each of which is independently substituted or unsubstituted, or hydrogen or halogen.

Embodiment 139. The compound of any one of embodiments 92-138, wherein R³ is hydrogen.

Embodiment 140. The compound of any one of embodiments 92-138, wherein R³ is —CH₂—COOH.

Embodiment 141. The compound of any one of embodiments 92-140, wherein R⁴ is C₁-C₆ alkyl, —(CH₂)_{q}COOR¹⁷, or —(CH₂)_{q}OR¹⁷, each of which is independently substituted or unsubstituted, or hydrogen or halogen.

Embodiment 142. The compound of any one of embodiments 92-141, wherein R⁴ is hydrogen.

Embodiment 143. The compound of any one of embodiments 92-142, wherein z is 0 and x is 1.

Embodiment 144. The compound of any one of embodiments 92-143, wherein R⁶ is hydrogen, CH₃, —(CH₂)₂OH, or —(CH₂)₂OCH₃.

Embodiment 145. The compound of any one of embodiments 92-144, wherein R⁶ is hydrogen.

Embodiment 146. The compound of any one of embodiments 92-144, wherein R⁶ is CH₃.

Embodiment 147. The compound of any one of embodiments 92-95, 98-105, 107, 108, 110-146, which is represented by the structure of formula (II):

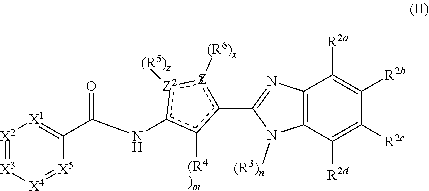

wherein each R^{2a}, R^{2b}, R^{2c} and R^{2d} independently has the same meaning as R² in formula (I).

Embodiment 148. The compound of any one of embodiments 92-95, 98-105, 107, 108, 110-147, which is represented by the structure of formula (III):

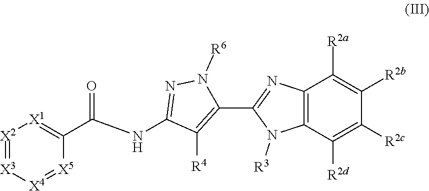

wherein each R^{2a}, R^{2b}, R^{2c} and R^{2d} independently has the same meaning as R² in formula (I).

Embodiment 149. The compound of any one of embodiments 92-95, 98-104, 107, 108, 110-148, which is represented by the structure of formula (IV):

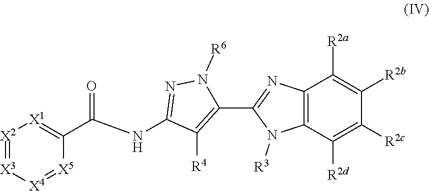

wherein each R^{2a}, R^{2b}, R^{2c} and R^{2d} independently has the same meaning as R² in formula (I).

Embodiment 150. The compound of any one of embodiments 147-149, wherein an R¹ is NR⁹R¹⁰, wherein R⁹ and R¹⁰ together with the nitrogen to which R⁹ and R¹⁰ are attached form an unsubstituted or substituted heterocyclic ring.

Embodiment 151. The compound of any one of embodiments 147-150, wherein NR⁹R¹⁰ is a 4-, 5-, 6-, or 7-membered ring, optionally containing at least one additional heteroatom, wherein the additional heteroatom is O, N or S.

Embodiment 152. The compound of any one of embodiments 147-151, wherein NR⁹R¹⁰ is piperidinyl, piperazinyl, azetidinyl, pyrrolidinyl, or morpholinyl, each of which is unsubstituted or substituted.

Embodiment 153. The compound of any one of embodiments 147-152, wherein NR⁹R¹⁰ is unsubstituted or substituted piperidinyl.

Embodiment 154. The compound of any one of embodiments 147-152, wherein NR⁹R¹⁰ is unsubstituted or substituted piperazinyl.

Embodiment 155. The compound of any one of embodiments 147-152, wherein NR⁹R¹⁰ is unsubstituted or substituted azetidinyl.

Embodiment 156. The compound of any one of embodiments 147-155, wherein each piperidinyl, piperazinyl, azetidinyl, pyrrolidinyl, or morpholinyl is independently substituted by —(CH₂)ₜOR¹⁹, wherein R¹⁹ is hydrogen, C₁-C₆ alkyl, or a N-, O-, or S-containing heterocyclyl, and t is 0, 1, 2, 3, 4, 5, or 6.

Embodiment 157. The compound of any one of embodiments 147-156, wherein each piperidinyl, piperazinyl, azetidinyl, pyrrolidinyl, or morpholinyl is independently substituted by C₁-C₆ alkyl.

Embodiment 158. The compound of any one of embodiments 147-157, wherein each piperidinyl, piperazinyl, azetidinyl, pyrrolidinyl, or morpholinyl is independently substituted with —N(R²⁰)₂, wherein each R²⁰ is independently hydrogen or a C₁-C₆ alkyl.

Embodiment 159. The compound of any one of embodiments 147-158, wherein each piperidinyl, piperazinyl, azetidinyl, pyrrolidinyl, or morpholinyl is independently substituted with —N(R²⁰)₂, wherein each R²⁰ independently together with the nitrogen atom to which each R²⁰ is bound form a ring, wherein the ring is substituted or unsubstituted.

Embodiment 160. The compound of embodiment 159, wherein the ring is pyrrolidine, piperidine, piperazine, morpholine.

Embodiment 161. The compound of any one of embodiments 147-160, wherein each piperidinyl, piperazinyl, azetidinyl, pyrrolidinyl, or morpholinyl is independently substituted by an oxygen-containing heterocyclic ring.

Embodiment 162. The compound of embodiment 161, wherein the oxygen-containing heterocyclic ring is oxetanyl.

Embodiment 163. The compound of any one of embodiments 92-95, 98-103, 105, 107, 108, 110-147, which is represented by the structure of formula (V):

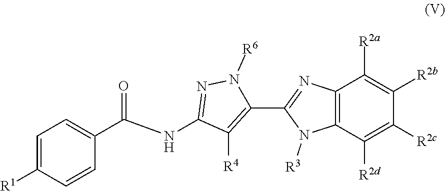

wherein each R^{2a}, R^{2b}, R^{2c} and R^{2d} independently has the same meaning as R² in formula (I).

Embodiment 164. The compound of embodiment 163, wherein an R¹ is NR⁹R¹⁰, wherein R⁹ and R¹⁰ together with the nitrogen to which R⁹ and R¹⁰ are attached form an unsubstituted or substituted heterocyclic ring.

Embodiment 165. The compound of embodiment 163 or 164, wherein NR⁹R¹⁰ is a 4-, 5-, 6-, or 7-membered ring, optionally containing at least one additional heteroatom, wherein the additional heteroatom is O, N or S.

Embodiment 166. The compound of any one of embodiments 163-165, wherein NR⁹R¹⁰ is piperidinyl, piperazinyl, azetidinyl, pyrrolidinyl, or morpholinyl, each of which is unsubstituted or substituted.

Embodiment 167. The compound of any one of embodiments 163-166, wherein NR⁹R¹⁰ is unsubstituted or substituted piperidinyl.

Embodiment 168. The compound of any one of embodiments 163-166, wherein NR⁹R¹⁰ is unsubstituted or substituted piperazinyl.

Embodiment 169. The compound of any one of embodiments 163-166, wherein NR⁹R¹⁰ is unsubstituted or substituted azetidinyl.

Embodiment 170. The compound of any one of embodiments 163-169, wherein each piperidinyl, piperazinyl, azetidinyl, pyrrolidinyl, or morpholinyl is independently substituted by —(CH₂)ₜOR¹⁹, wherein R¹⁹ is hydrogen, C₁-C₆ alkyl, or a N-, O-, or S-containing heterocyclyl, and t is 0, 1, 2, 3, 4, 5, or 6.

Embodiment 171. The compound of any one of embodiments 163-170, wherein each piperidinyl, piperazinyl, azetidinyl, pyrrolidinyl, or morpholinyl is independently substituted by C₁-C₆ alkyl.

Embodiment 172. The compound of any one of embodiments 163-171, wherein each piperidinyl, piperazinyl, azetidinyl, pyrrolidinyl, or morpholinyl is independently substituted with substituted C₁-C₆alkyl.

Embodiment 173. The compound of any one of embodiments 163-172, wherein each piperidinyl, piperazinyl, azetidinyl, pyrrolidinyl, or morpholinyl is independently substituted —N(R²⁰)₂, wherein each R²⁰ is independently hydrogen or a C₁-C₆ alkyl.

Embodiment 174. The compound of any one of embodiments 163-173, wherein each piperidinyl, piperazinyl, azetidinyl, pyrrolidinyl, or morpholinyl is independently substituted by an oxygen-containing heterocyclic ring.

Embodiment 175. The compound of embodiment 174, wherein the oxygen-containing heterocyclic ring is oxetanyl.

Embodiment 176. The compound of any one of embodiments 163-166, wherein R⁶ is methyl and R¹ is piperidinyl, piperazinyl, morpholinyl or pyrrolidinyl.

Embodiment 177. The compound of any one of embodiments 92-95, 98-101, 103-108, 110-115, 117-121, 123-162, which is represented by the structure of formula (VI):

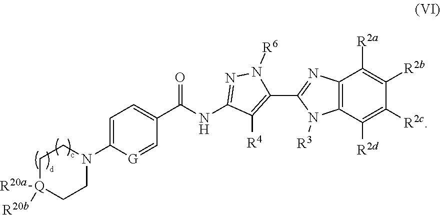

wherein
G is N or CH;
Q is N or C;
each R^{2a}, R^{2b}, R^{2c} and R^{2d} independently has the same meaning as R² in formula (I);
each R^{20a} and R^{20b} is independently:
i) —(CH₂)ₜOR¹⁹, wherein R¹⁹ is hydrogen, a C₁-C₆ alkyl, wherein the C₁-C₆ alkyl is unsubstituted or substituted, or a N, O, or S-containing heterocyclyl;
ii) —C(O)R^{19a}, wherein R^{19a} is C₁-C₆ alkyl or —(CH₂)ₓOR²¹, wherein R²¹ is C₁-C₆ alkyl that is unsubstituted or substituted with NH₂ or OH, wherein x is 0 or 1;
iii) —NR²²R²³, wherein R²² and R²³ are independently C₁-C₆ alkyl, or R²² and R²³, together with the nitrogen to which are R²² and R²³ are attached, form an unsubstituted or substituted heterocyclic ring;
iv) —OR²⁴, wherein R²⁴ is C₁-C₆ alkyl;
v) an oxygen-containing heterocyclic ring;
vi) —C₁-C₆ alkyl, —OH, or absent; or
vii) hydrogen, provided that at least one of R^{20a} and R^{20b} is not hydrogen; or R^{20a} and R^{20b} together with Q form an oxygen-containing heterocyclic ring;
c is 0 or 1;
d is 0 or 1; and
t is 0, 1, 2, 3, 4, 5 or 6;
   or a pharmaceutically-acceptable salt or a tautomer thereof.

Embodiment 178. The compound of embodiment 177, which is represented by the structure of formula (VII):

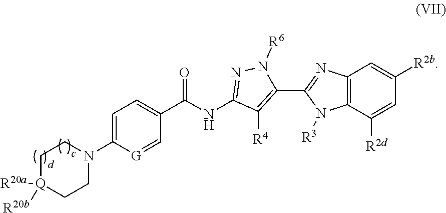

Embodiment 179. A compound that is N-[5-(1H-benzimidazol-2-yl)-1-methyl-pyrazol-3-yl]-4-morpholino-benzamide, or a pharmaceutically-acceptable salt or a tautomer thereof.

Embodiment 180. A compound that is N-[5-(1H-benzimidazol-2-yl)-1-methyl-pyrazol-3-yl]-6-[4-(hydroxymethyl)-1-piperidyl]pyridine-3-carboxamide, or a pharmaceutically-acceptable salt or a tautomer thereof.

Embodiment 181. A compound that is N-[5-(1H-benzimidazol-2-yl)-1H-pyrazol-3-yl]-4-methylsulfonyl-benzamide, or a pharmaceutically-acceptable salt or a tautomer thereof.

Embodiment 182. A compound that is N-[5-(1H-benzimidazol-2-yl)-1-methyl-pyrazol-3-yl]-6-(3-methoxyazetidin-1-yl)pyridine-3-carboxamide, or a pharmaceutically-acceptable salt or a tautomer thereof.

Embodiment 183. A compound that is N-[5-(1H-benzimidazol-2-yl)-1-[(4-methoxyphenyl)methyl]pyrazol-3-yl]-3-chloro-4-methoxy-benzamide, or a pharmaceutically-acceptable salt or a tautomer thereof.

Embodiment 184. A compound that is N-[5-(1H-benzimidazol-2-yl)-1H-pyrazol-3-yl]-6-(1-oxa-8-azaspiro[4.5]decan-8-yl)pyridine-3-carboxamide, or a pharmaceutically-acceptable salt or a tautomer thereof, or a pharmaceutically-acceptable salt or a tautomer thereof.

Embodiment 185. A compound that is 6-[4-(dimethylamino)-1-piperidyl]-N-[5-(5-fluoro-1H-benzimidazol-2-yl)-1-methyl-pyrazol-3-yl]pyridine-3-carboxamide, or a pharmaceutically-acceptable salt or a tautomer thereof.

Embodiment 186. A compound that is N-[5-(1H-benzimidazol-2-yl)-1H-pyrazol-3-yl]-6-[4-(hydroxymethyl)-1-piperidyl]pyridine-3-carboxamide, or a pharmaceutically-acceptable salt or a tautomer thereof.

Embodiment 187. A compound that is N-[5-(1H-1,3-benzodiazol-2-yl)-1H-pyrazol-3-yl]-4-[4-(2-hydroxyethyl)piperazin-1-yl]benzamide, or a pharmaceutically-acceptable salt or a tautomer thereof.

Embodiment 188. A compound that is N-[5-(1H-benzimidazol-2-yl)-1-methyl-pyrazol-3-yl]-6-[4-(oxetan-3-yl)piperazin-1-yl]pyridine-3-carboxamide, or a pharmaceutically-acceptable salt or a tautomer thereof.

Embodiment 189. A compound that is N-[5-(5-chloro-1H-benzimidazol-2-yl)-1H-pyrazol-3-yl]-6-(4-hydroxy-1-piperidyl)pyridine-3-carboxamide, or a pharmaceutically-acceptable salt or a tautomer thereof.

Embodiment 190. A compound that is N-[5-(1H-benzimidazol-2-yl)-1-methyl-pyrazol-3-yl]-6-(4-methylpiperazin-1-yl)pyridine-3-carboxamide, or a pharmaceutically-acceptable salt or a tautomer thereof.

Embodiment 191. A compound that is N-[5-(1H-benzimidazol-2-yl)-1H-pyrazol-3-yl]-4-[(3R)-3-hydroxypyrrolidin-1-yl]benzamide , or a pharmaceutically-acceptable salt or a tautomer thereof.

Embodiment 192. A compound that is N-[5-(1H-benzimidazol-2-yl)-1-methyl-pyrazol-3-yl]-4-[4-(oxetan-3-yl)piperazin-1-yl]benzamide, or a pharmaceutically-acceptable salt or a tautomer thereof.

Embodiment 193. A compound that is N-[5-(1H-benzimidazol-2-yl)-1H-pyrazol-3-yl]-4-[4-(oxetan-3-yl)piperazin-1-yl]benzamide, or a pharmaceutically-acceptable salt or a tautomer thereof.

Embodiment 194. A compound that is N-[5-(1H-benzimidazol-2-yl)-1H-pyrazol-3-yl]-3-cyano-4-(2-hydroxyethoxy)benzamide, or a pharmaceutically-acceptable salt or a tautomer thereof.

Embodiment 195. A compound that is N-[5-(5-fluoro-1H-benzimidazol-2-yl)-1-methyl-pyrazol-3-yl]-6-(4-methoxy-1-piperidyl)pyridine-3-carboxamide, or a pharmaceutically-acceptable salt or a tautomer thereof.

Embodiment 196. A compound that is N-[5-(1H-benzimidazol-2-yl)-1-methyl-pyrazol-3-yl]-4-(2-hydroxyethoxy)-3-(trifluoromethyl)benzamide, or a pharmaceutically-acceptable salt or a tautomer thereof.

Embodiment 197. A compound that is N-[5-(1H-benzimidazol-2-yl)-1H-pyrazol-3-yl]-2-(4-methylpiperazin-1-yl)pyrimidine-5-carboxamide, or a pharmaceutically-acceptable salt or a tautomer thereof.

Embodiment 198. A compound that is N-[5-(1H-benzimidazol-2-yl)-1-methyl-pyrazol-3-yl]-6-[(3R,5S)-3,5-dimethylpiperazin-1-yl]pyridine-3-carboxamide, or a pharmaceutically-acceptable salt or a tautomer thereof.

Embodiment 199. A compound that is 6-(4-acetylpiperazin-1-yl)-N-[5-(1H-benzimidazol-2-yl)-1-methyl-pyrazol-3-yl]pyridine-3-carboxamide, or a pharmaceutically-acceptable salt or a tautomer thereof.

Embodiment 200. A pharmaceutical composition comprising a compound of any one of embodiments 1-199, and a pharmaceutically-acceptable excipient in a unit dosage form.

Embodiment 201. A method of treating a condition, the method comprising administering to a subject in need thereof a therapeutically-effective amount of a compound of any one of embodiments 1-199.

Embodiment 202. The method of embodiment 201, wherein the condition is diabetes.

Embodiment 203. The method of embodiment 202, wherein the condition is insulin-dependent diabetes.

Embodiment 204. The method of embodiment 203, wherein the insulin-dependent diabetes is type 1 diabetes mellitus (T1DM).

Embodiment 205. The method of embodiment 203, wherein the insulin-dependent diabetes is diabetes secondary to pancreatectomy.

Embodiment 206. The method of embodiment 201, wherein the condition is non-insulin-dependent diabetes.

Embodiment 207. The method of embodiment 201, wherein the condition is type 2 diabetes or gestational diabetes.

Embodiment 208. A method of treating a condition, the method comprising administering to a subject in need thereof a therapeutically-effective amount of a pharmaceutical composition of embodiment 199.

Embodiment 209. The method of embodiment 208, wherein the condition is diabetes.

Embodiment 210. The method of embodiment 209, wherein the condition is insulin-dependent diabetes.

Embodiment 211. The method of embodiment 210, wherein the insulin-dependent diabetes is type 1 diabetes mellitus (T1DM).

Embodiment 212. The method of embodiment 210, wherein the insulin-dependent diabetes is diabetes secondary to pancreatectomy.

Embodiment 213. The method of embodiment 209, wherein the condition is non-insulin-dependent diabetes.

Embodiment 214. The method of embodiment 209, wherein the condition is type 2 diabetes or gestational diabetes.

Embodiment 215. The compound of any one of embodiments 1-199, for use in a method of treating a condition in a subject in need thereof.

Embodiment 216. The pharmaceutical composition of embodiment 200, for use in a method of treating a condition in a subject in need thereof.

Embodiment 217. The pharmaceutical composition for use of embodiment 216, wherein the condition is diabetes.

Embodiment 218. The pharmaceutical composition for use of embodiment 217, wherein the condition is insulin-dependent diabetes.

Embodiment 219. The pharmaceutical composition for use of embodiment 218, wherein the insulin-dependent diabetes is type 1 diabetes mellitus (T1DM).

Embodiment 220. The pharmaceutical composition for use of embodiment 218, wherein the insulin-dependent diabetes is diabetes secondary to pancreatectomy.

Embodiment 221. The pharmaceutical composition for use of embodiment 217, wherein the condition is non-insulin-dependent diabetes.

Embodiment 222. The pharmaceutical composition for use of embodiment 217, wherein the condition is type 2 diabetes or gestational diabetes.

What is claimed is:

1. A compound of formula (Ib) 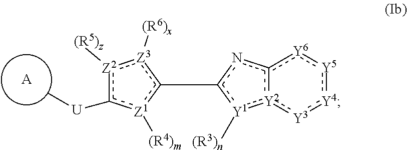
wherein:
A is an aryl or heteroaryl of the formula: 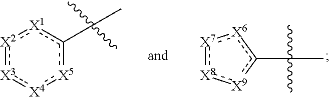
each [symbol] is independently a single bond or a double bond;
each of X¹, X², X³, X⁴, X⁵, X⁶, X⁷, X⁸, and X⁹ is independently CR¹, N, NR¹, O, or S;
Y¹ is C, N, or O wherein, (i) when Y¹ is O, n is 0; (ii) when Y¹ is N, n is 0 or 1; and
(iii) when Y¹ is C, n is 1;
Y² is C or N;
each of Y³, Y⁴, Y⁵, and Y⁶ is independently CR², N, NR², O, or S;
Z¹ is C, N, or O; wherein (i) when Z¹ is O, m is 0, (ii) when Z¹ is N, m is 0 or 1, and (iii) wherein Z¹ is C, m is 1;
Z² is C, N, or O; wherein (i) when Z² is O, z is 0; (ii) when Z² is N, z is 0 or 1; and (iii) when Z² is C, z is 1;
Z³ is C, N, or O; wherein (i) when Z³ is O, x is 0; (ii) when Z³ is N, x is 0 or 1; and (iii) when Z³ is C, x is 1;
U is —C(═O)—N(R^{7a})—, wherein R^{7a} is hydrogen or C₁-C₆ alkyl;
each R¹ is independently alkyl, alkenyl, alkynyl, cycloalkyl, aryl, arylalkyl, alkylaryl, heterocyclyl, or heteroaryl, —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵, or —S(CH₂)_{q}R¹⁶, hydrogen, or halogen, wherein
when (i) each of X¹, X², X³, X⁴, and X⁵ is independently CR¹, or (ii) each of X¹, X², X⁴, and X⁵ is independently CR¹, and X³ is N, then:
when (i) at least one of R¹ is independently Br, CN, —SCH₃, —S(O)₂CH₃, —O(CH₂)₂OCH₃, —O(CH₂)₃OCH₃, —O(CH₂)₂morpholinyl, —OCH₂COOH, —OCH₂C(═O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OH, —NHC(CH₃)—CH₂OCH₃, —SCH₂CH₂OCH₃; or
(ii) at least one of R¹ is NR⁹R¹⁰, wherein NR⁹R¹⁰ is piperidinyl, piperazinyl or pyrrolidinyl, each of which is substituted by —(CH₂)ₜOR¹⁹, wherein R¹⁹ is hydrogen, C₁-C₆ alkyl, or N-, O-, or S-containing heterocyclyl, and t is 0, 1, 2, 3, 4, 5, or 6;
each R² is independently alkyl, alkenyl, alkynyl, cycloalkyl, aryl, arylalkyl, alkylaryl, heterocyclyl, or heteroaryl, each of which is independently substituted or unsubstituted, or hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁵ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, or heteroaryl, each of which is independently unsubstituted or substituted, or —OH, —OR⁷, —NH₂, —NHR⁸, —NR⁹R¹⁰, —(CH₂)_{q}COOR¹⁷,—(CH₂)_{q}N(R¹⁷,—COOR¹⁸, or hydrogen;
each of R¹⁷ and R¹⁸ is independently hydrogen or alkyl; and
each q is independently 1, 2, 3, 4, 5, or 6,
or a pharmaceutically-acceptable salt or a tautomer thereof.

2. The compound of claim 1, wherein each of X¹, X², X³, X⁴, and X⁵ is independently CR¹, N, or NR¹.

3. The compound of claim 1, wherein each of X¹, X², X³, X⁴, and X⁵ is CR¹.

4. The compound of claim 1, wherein one of X¹, X², X³, X⁴, and X⁵ is N or NR¹ and the others are CR¹.

5. The compound of claim 1, wherein two of X¹, X², X³, X⁴, and X⁵ are N or NR¹ and the others are CR¹.

6. (canceled)

7. The compound of claim 1, wherein A is

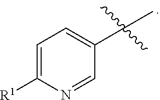

8. (canceled)

9. The compound of claim 1, which is represented by the structure of formula (Vb) 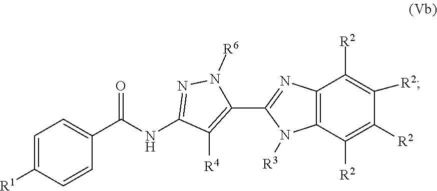
wherein:
R¹ is independently Br, CN, —SCH₃, —S(O)₂CH₃, —O(CH₂)₂OCH₃, —O(CH₂)₃OCH₃, —O(CH₂)₂morpholinyl, —OCH₂COOH, —OCH₂C(═O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OH, —NHC(CH₃)—CH₂OCH₃, —SCH₂CH₂OCH₃; or
R¹ is NR⁹R¹⁰, wherein NR⁹R¹⁰ is piperidinyl, piperazinyl or pyrrolidinyl, each of which is substituted by —(CH₂)ₜOR¹⁹, wherein R¹⁹ is hydrogen, substituted or unsubstituted C₁-C₆ alkyl, or N-, O-, or S-containing heterocyclyl, and t is 0, 1, 2, 3, 4, 5, or 6.

10. The compound of claim 9, wherein R¹⁹ is a substituted C₁-C₆ alkyl.

11. The compound of claim 9, wherein at least one R¹ is independently Br, CN, —SCH₃, —S(O)₂CH₃, —O(CH₂)₂OCH₃, —O(CH₂)₃OCH₃, —O(CH₂)₂morpholinyl, —OCH₂COOH, —OCH₂C(—O)OCH₂CH₃, NH(CH₂)₂OH, —NH(CH₂)₂OCH₃, NH(CH₂)₂OCH₂CH₃, —O(CH₂)₂OH, —NHC(CH₃)—CH₂OCH₃, or —NHC(CH₃)—CH₂OCH₃, or —SCH₂CH₂OCH₃.

12. The compound of claim 9, wherein at least one R¹ is NR⁹R¹⁰, wherein NR⁹R¹⁰ is piperidinyl, piperazinyl, azetidinyl, pyrrolidinyl, or morpholinyl, each of which is independently substituted by —(CH₂)ₜOR¹⁹, wherein R¹⁹ is hydrogen, C₁-C₆ alkyl, or a N-, O-, or S-containing heterocyclyl, and t is 0, 1, 2, 3, 4, 5, or 6.

13. The compound of claim 12, wherein each piperidinyl, piperazinyl, azetidinyl, pyrrolidinyl, or morpholinyl is independently substituted by an oxygen-containing heterocyclic ring.

14. The compound of claim 13, wherein the oxygen-containing heterocyclic ring is oxetanyl.

15. The compound of claim 1, wherein each R² is hydrogen, halogen, or C₁-C₆ alkoxy.

16. The compound of claim 1, wherein R³ is C₁-C₆ alkyl, —(CH₂)_{q}COOR¹⁷, or —(CH₂)_{q}OR¹⁷, each of which is independently substituted or unsubstituted, or hydrogen or halogen.

17. The compound of claim 16, wherein R³ is hydrogen.

18. The compound of claim 16, wherein R³ is —CH₂—COOH.

19. The compound of claim 1, wherein R⁴ is C₁-C₆ alkyl, —(CH₂)_{q}COOR¹⁷, or —(CH₂)_{q}OR¹⁷, each of which is independently substituted or unsubstituted, or hydrogen or halogen.

20. The compound of claim 19, wherein R⁴ is hydrogen.

21. The compound of claim 1, wherein R⁶ is hydrogen, CH₃, —(CH₂)₂OH, or —(CH₂)₂OCH₃.

22. The compound of claim 21, wherein R⁶ is hydrogen.

23. The compound of claim 21, wherein R⁶ is CH₃.

24. A compound which is represented by the structure of formula (VIa): 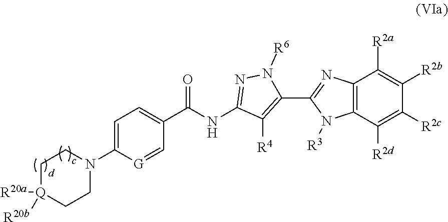
wherein
G is N or CH;
Q is N or C;
each R^{2a}, R^{2b}, R^{2c} and R^{2d} is independently alkyl, alkenyl, alkynyl, cycloalkyl, aryl, arylalkyl, alkylaryl, heterocyclyl, or heteroaryl, each of which is independently substituted or unsubstituted, or —OR⁷, —CN, —NH₂, —NHR⁸, —NR⁹R¹⁰, —SR¹¹, —S(O)₂R¹², —NHC(O)R¹³, —O(CH₂)_{q}R¹⁴, —NH(CH₂)_{q}R¹⁵,—S(CH₂)_{q}R¹⁶, hydrogen or halogen;
R³ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁴ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
R⁶ is alkyl, arylalkyl, alkylaryl, —(CH₂)_{q}COOR¹⁷, —(CH₂)_{q}N(R¹⁷)₂, or —(CH₂)_{q}OR¹⁷, each of which is independently substituted or unsubstituted, or hydrogen or halogen;
each R⁷ is independently alkyl, alkenyl, alkynyl, cycloalkyl, aryl, arylalkyl, alkylaryl, heterocyclyl, or heteroaryl, each of which is independently unsubstituted or substituted, or hydrogen;
each R⁸ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R⁹ and R¹⁰ is independently alkyl, alkenyl, alkynyl, cycloalkyl, aryl, arylalkyl, alkylaryl, heterocyclyl, or heteroaryl, each of which is independently unsubstituted or substituted, or hydrogen; or R⁹ and R¹⁰ together with the nitrogen atom to which R⁹ and R¹⁰ are attached form a heterocyclic or heteroaromatic ring, which is unsubstituted or substituted;
each R¹¹, R¹², R¹³, R¹⁴, R¹⁵, and R¹⁶ is independently alkyl, alkenyl, alkynyl, cycloalkyl, aryl, arylalkyl, alkylaryl, heterocyclyl, or heteroaryl, each of which is independently unsubstituted or substituted, or hydrogen;
each of R¹⁷ and R¹⁸ is independently hydrogen or alkyl;
each R^{20a} and R^{20b} is independently:
i) —(CH₂)ₜOR¹⁹, wherein R¹⁹ is hydrogen, a C₁-C₆ alkyl, wherein the C₁-C₆ alkyl is unsubstituted or substituted, or a N, O, or S-containing heterocyclyl;
ii) —C(O)R^{19a}, wherein R^{19a} is C₁-C₆ alkyl or —(CH₂)ₓOR²¹, wherein R²¹ is C₁-C₆ alkyl that is unsubstituted or substituted with NH₂ or OH, wherein x is 0 or 1;
iii) —NR²²R²³, wherein R²² and R²³ are independently C₁-C₆ alkyl, or R²² and R²³, together with the nitrogen to which are R²² and R²³ are attached, form an unsubstituted or substituted heterocyclic ring;
iv) —OR²⁴, wherein R²⁴ is C₁-C₆ alkyl;
v) an oxygen-containing heterocyclic ring;
vi) —C₁-C₆ alkyl, —OH, or absent; or
vii) hydrogen, provided that at least one of R^{20a} and R^{20b} is not hydrogen;
or R^{20a} and R^{20b} together with Q form an oxygen-containing heterocyclic ring;
c is 0 or 1;
d is 0 or 1;
each q is independently 1, 2, 3, 4, 5, or 6, and
t is 0, 1, 2, 3, 4, 5 or 6;
or a pharmaceutically-acceptable salt or a tautomer thereof.

25. The compound of claim 24, which is represented by the structure of formula (VIIb):

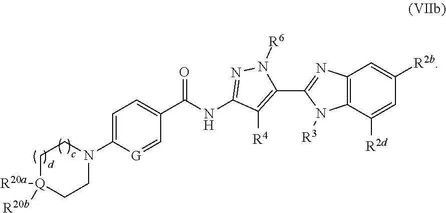

26-222. (canceled)
